(19) United States
(12) Patent Application Publication
(10) Pub. No.: US 2025/0099588 A1
(43) Pub. Date: Mar. 27, 2025

(54) CYTOKINE ASSOCIATED TUMOR INFILTRATING LYMPHOCYTES COMPOSITIONS AND METHODS

(71) Applicant: Iovance Biotherapeutics, Inc., San Carlos, CA (US)

(72) Inventors: Frederick G. VOGT, West Norriton, PA (US); Maria FARDIS, San Carlos, CA (US); Cecile CHARTIER-COURTAUD, Palo Alto, CA (US); Rafael CUBAS, Tampa, FL (US); Yongliang ZHANG, Wesley Chapel, FL (US); Pasquale Patrick INNAMARATO, IV, Ruskin, FL (US); Nathan GILBERT, Brandon, FL (US)

(21) Appl. No.: 18/832,493

(22) PCT Filed: Jan. 27, 2023
(86) PCT No.: PCT/US2023/061474

§ 371 Date: Jul. 23, 2024

Related U.S. Application Data
(60) Provisional application No. 63/429,114, filed on Nov. 30, 2022.
(60) Provisional application No. 63/382,493, filed on Nov. 4, 2022.
(60) Provisional application No. 63/394,267, filed on Aug. 1, 2022.
(60) Provisional application No. 63/356,933, filed on Jun. 29, 2022.
(60) Provisional application No. 63/304,498, filed on Jan. 28, 2022.

(51) Int. Cl. A61K 39/00; C07K 14/54; C12N 5/0783; C12N 15/86

(52) U.S. Cl. CPC A61K 40/4234; A61K 40/11; C07K 14/54; C07K 14/5443; C12N 5/0636; C12N 15/86; C12N 2501/2302; C12N 2501/515; C12N 2502/30; C12N 2740/15043

(57) ABSTRACT

Provided herein are compositions and methods for the treatment of cancers using modified TILs, wherein the modified TILs include one or more immunomodulatory agents (e.g., cytokines) associated with their cell surface. The immunomodulatory agents associated with the TILs provide a localized immunostimulatory effect that can advantageously enhance TIL survival, proliferation and/or anti-tumor activity in a patient recipient. As such, the compositions and methods disclosed herein provide effective cancer therapies.

### CROSS-REFERENCE TO RELATED APPLICATIONS

This application claims priority to U.S. Provisional Application No. 63/304,498, filed Jan. 28, 2022, U.S. Provisional Application No. 63/356,933, filed Jun. 29, 2022, U.S. Provisional Application No. 63/394,267, filed Aug. 1, 2022, U.S. Provisional Application No. 63/382,493, filed Nov. 4, 2022, and U.S. Provisional Application No. 63/429,114, filed Nov. 30, 2022, the disclosures of which are herein incorporated by reference in their entireties.

### BACKGROUND

Adoptive cell therapy utilizing TILs cultured ex vivo by the Rapid Expansion Protocol (REP) has produced successful adoptive cell therapy following host immunosuppression in patients with cancer. In some instances, however, the survival and anti-tumor activity of the transferred TILs can decrease following transfer to the patient.

Administration of supporting immunostimulatory agents (e.g., cytokines) have been explored to enhance T cell therapies. Such immunostimulatory agents, however, require high systemic doses that can lead to undesirable toxicity.

Thus, there remains a need for improved TIL therapies for the treatment of cancers.

### BRIEF SUMMARY

Provided herein are compositions and methods for the treatment of cancers using modified TILs, wherein the modified TILs include one or more immunomodulatory agents (e.g., cytokines) associated with their cell surface. The immunomodulatory agents associated with the TILs provide a localized immunostimulatory effect that can advantageously enhance TIL survival, proliferation and/or anti-tumor activity in a patient recipient. As such, the compositions and methods disclosed herein provide effective cancer therapies.

In one aspect, provided herein is a method of treating a cancer in a patient or subject in need thereof comprising administering a population of tumor infiltrating lymphocytes (TILs), optionally wherein the patient or subject has received at least one prior therapy, wherein a portion of the population of TILs are modified TILs each comprising an immunomodulatory composition associated with its surface membrane.

In one aspect, provided herein is a method of treating a cancer in a patient or subject in need thereof comprising administering a population of modified tumor infiltrating lymphocytes (TILs), the method comprising the steps of:
(a) obtaining and/or receiving a first population of TILs from a tumor resected from the subject or patient by processing a tumor sample obtained from the subject into multiple tumor fragments;
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting therapeutic population of TILs obtained from step (d), wherein the transition from step (d) to step (e) occurs without opening the system;
(f) transferring the harvested TIL population from step (e) to an infusion bag, wherein the transfer from step (e) to (f) occurs without opening the system;
(g) cryopreserving the infusion bag comprising the harvested TIL population from step (f) using a cryopreservation process;
(h) administering a therapeutically effective dosage of the third population of TILs from the infusion bag in step (g) to the subject; and
(i) modifying a portion of the first, second, or third population of TILs at any time prior to the administering step (h) to generate modified TILs each comprising an immunomodulatory composition associated with its surface membrane.

In one aspect, provided herein is a method of treating a cancer in a patient or subject in need thereof comprising administering a population of tumor infiltrating lymphocytes (TILs), the method comprising the steps of:
(a) obtaining a first population of TILs from a tumor resected from a subject by processing a tumor sample obtained from the subject into multiple tumor fragments;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system;
(g) cryopreserving the infusion bag comprising the harvested TIL population from step (f) using a cryopreservation process;
(h) administering a therapeutically effective dosage of the third population of TILs from the infusion bag in step (g) to the subject; and
(i) modifying a portion of the first, second, or third population of TILs at any time prior to the administering step (h) to generate modified TILs each comprising an immunomodulatory composition associated with its surface membrane.

In one aspect, provided herein is a method of treating a cancer in a patient or subject in need thereof comprising administering a population of tumor infiltrating lymphocytes (TILs), the method comprising the steps of:
(a) obtaining and/or receiving a first population of TILs from surgical resection, needle biopsy, core biopsy, small biopsy, or other means for obtaining a sample that contains a mixture of tumor and TIL cells from the cancer in the patient or subject,
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system;
(g) cryopreserving the infusion bag comprising the harvested TIL population from step (f) using a cryopreservation process;
(h) administering a therapeutically effective dosage of the third population of TILs from the infusion bag in step (g) to the subject; and
(i) modifying a portion of the first, second, or third population of TILs at any time prior to the administering step (h) to generate modified TILs each comprising an immunomodulatory composition associated with its surface membrane.

In another aspect, provided herein is a method of treating a cancer in a patient or subject in need thereof comprising administering a population of modified tumor infiltrating lymphocytes (TILs), the method comprising the steps of:
(a) resecting a tumor from the subject or patient, the tumor comprising a first population of TILs, optionally from surgical resection, needle biopsy, core biopsy, small biopsy, or other means for obtaining a sample that contains a mixture of tumor and TIL cells from the cancer;
(b) processing the tumor into multiple tumor fragments and adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system;
(g) cryopreserving the infusion bag comprising the harvested TIL population from step (f) using a cryopreservation process;
(h) administering a therapeutically effective dosage of the third population of TILs from the infusion bag in step (g) to the subject or patient with the cancer; and
(i) modifying a portion of the first, second, or third population of TILs at any time prior to the administering step (h) to generate modified TILs each comprising an immunomodulatory composition associated with its surface membrane.

In other aspect, provided herein is a method of treating a cancer in a patient or subject in need thereof comprising administering a population of tumor infiltrating lymphocytes (TILs), the method comprising the steps of:
(a) obtaining and/or receiving a first population of TILs from surgical resection, needle biopsy, core biopsy, small biopsy, or other means for obtaining a sample that contains a mixture of tumor and TIL cells from the subject or patient;
(c) contacting the first population of TILs with a first cell culture medium;
(d) performing an initial expansion (or priming first expansion) of the first population of TILs in the first cell culture medium to obtain a second population of TILs, wherein the first cell culture medium comprises IL-2, optionally OKT-3 (anti-CD3 antibody), and optionally antigen presenting cells (APCs), where the priming first expansion occurs for a period of 1 to 8 days;
(e) performing a rapid second expansion of the second population of TILs in a second cell culture medium to obtain a third population of TILs; wherein the second cell culture medium comprises IL-2, OKT-3 (anti-CD3 antibody), and APCs; and wherein the rapid expansion is performed over a period of 14 days or less, optionally the rapid second expansion can proceed for 1 day, 2 days, 3 days, 4, days, 5 days, 6 days, 7 days, 8 days, 9 days or 10 days after initiation of the rapid second expansion;
(f) harvesting the third population of TILs;
(g) administering a therapeutically effective portion of the third population of TILs to the subject or patient with the cancer; and
(h) modifying a portion of the first, second, or third population of TILs at any time prior to the administering step (g) to generate modified TILs each comprising an immunomodulatory composition associated with its surface membrane.

In another aspect, provided herein is a method of treating a cancer in a patient or subject in need thereof comprising administering a population of tumor infiltrating lymphocytes (TILs), the method comprising the steps of:
(a) resecting a tumor from the cancer in the subject or patient, the tumor comprising a first population of TILs, optionally from surgical resection, needle biopsy, core biopsy, small biopsy, or other means for obtaining a sample that contains a mixture of tumor and TIL cells from the cancer;
(b) fragmenting the tumor into tumor fragments;
(c) contacting the tumor fragments with a first cell culture medium;
(d) performing an initial expansion (or priming first expansion) of the first population of TILs in the first cell culture medium to obtain a second population of TILs, wherein the first cell culture medium comprises IL-2, optionally OKT-3 (anti-CD3 antibody), and optionally antigen presenting cells (APCs), where the priming first expansion occurs for a period of 1 to 8 days;
(e) performing a rapid second expansion of the second population of TILs in a second cell culture medium to obtain a third population of TILs; wherein the second cell culture medium comprises IL-2, OKT-3 (anti-CD3 antibody), and APCs; and wherein the rapid expansion is performed over a period of 14 days or less, optionally the rapid second expansion can proceed for 1 day, 2 days, 3 days, 4, days, 5 days, 6 days, 7 days, 8 days, 9 days or 10 days after initiation of the rapid second expansion;
(f) harvesting the third population of TILs;
(g) administering a therapeutically effective portion of the third population of TILs to the subject or patient with the cancer; and
(h) modifying a portion of the first, second, or third population of TILs at any time prior to the administering step (g) to generate modified TILs each comprising an immunomodulatory composition associated with its surface membrane.

In one aspect, provided herein is a method for expanding tumor infiltrating lymphocytes (TILs) into a therapeutic population of TILs comprising:
(a) obtaining and/or receiving a first population of TILs from a tumor resected from a cancer in a subject by processing a tumor sample obtained from the tumor into multiple tumor fragments;
(b) selecting PD-1 positive TILs from the first population of TILs in step (a) to obtain a PD-1 enriched TIL population;
(c) performing a priming first expansion by culturing the PD-1 enriched TIL population in a first cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7/8 days to obtain the second population of TILs, wherein the second population of TILs is greater in number than the first population of TILs;
(d) performing a rapid second expansion by culturing the second population of TILs in a second culture medium comprising IL-2, OKT-3, and APCs, to produce a therapeutic population of TILs, wherein the number of APCs added in the rapid second expansion is at least twice the number of APCs added in step (b), wherein the rapid second expansion is performed for a second period of about 1 to 11 days to obtain the therapeutic population of TILs, wherein the third population of TILs is a therapeutic population of TILs, wherein the rapid second expansion is performed in a container comprising a second gas-permeable surface area;
(e) harvesting the therapeutic population of TILs obtained from step (d);
(f) transferring the harvested TIL population from step (e) to an infusion bag, and
(g) modifying a portion of the first, second, or third population of TILs at any time during the method to generate modified TILs each comprising an immunomodulatory composition associated with its surface membrane.

Provided herein is a method of expanding tumor infiltrating lymphocytes (TILs) into a therapeutic population of TILs, the method comprising the steps of:
(a) obtaining and/or receiving a first population of TILs from a tumor resected from a cancer in a subject or patient by processing a tumor sample obtained from the tumor into multiple tumor fragments;
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting therapeutic population of TILs obtained from step (d), wherein the transition from step (d) to step (e) occurs without opening the system;
(f) transferring the harvested TIL population from step (e) to an infusion bag, wherein the transfer from step (e) to (f) occurs without opening the system; and
(g) modifying a portion of the first, second or third population of TILs at any time prior to the transfer to the infusion bag in step (f) to generate modified TILs each comprising an immunomodulatory composition associated with its surface membrane.

Provided herein is a method of expanding tumor infiltrating lymphocytes (TILs) into a therapeutic population of TILs, the method comprising the steps of:
(a) obtaining a first population of TILs from a tumor resected from a cancer in a subject by processing a tumor sample obtained from the tumor into multiple tumor fragments;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system; and
(g) modifying a portion of the first, second, or third population of TILs at any time prior to the transfer to the infusion bag in step (f) to generate modified TILs each comprising an immunomodulatory composition associated with its surface membrane.

In another aspect, provided herein is a method of expanding tumor infiltrating lymphocytes (TILs) into a therapeutic population of TILs, the method comprising the steps of:
(a) obtaining and/or receiving a first population of TILs from surgical resection, needle biopsy, core biopsy, small biopsy, or other means for obtaining a sample that contains a mixture of tumor and TIL cells from a cancer in a patient or subject,
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system; and
(g) modifying a portion of the first, second, or third population of TILs at any time prior to the transfer to the infusion bag in step (f) to generate modified TILs each comprising an immunomodulatory composition associated with its surface membrane.

In one aspect, provided herein is a method of expanding tumor infiltrating lymphocytes (TILs) to a therapeutic population of TILs, the method comprising the steps of:
(a) resecting a tumor from a cancer in a subject or patient, the tumor comprising a first population of TILs, optionally from surgical resection, needle biopsy, core biopsy, small biopsy, or other means for obtaining a sample that contains a mixture of tumor and TIL cells from the cancer;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system; and
(g) modifying a portion of the first, second, or third population of TILs at any time prior to the transfer to infusion bag in step (f) to generate modified TILs each comprising an immunomodulatory composition associated with its surface membrane.

In some embodiments, of the methods provided herein, the first expansion is divided into a first step and a second step, wherein the method further comprises performing the first step of the first expansion by culturing the first population of TILs in a cell culture medium containing IL-2 to produce TILs that egress from the tumor fragments or sample, separating TILs that remain in the tumor fragments or sample from TILs that egressed from the tumor fragments or sample, optionally digesting the tumor fragments or sample to produce a tumor digest, and performing the second step of the first expansion by culturing in the cell culture medium the TILs remaining in the tumor fragments or sample or tumor digest to produce the second population of TILs.

In one aspect, provided herein is a method of expanding tumor infiltrating lymphocytes (TILs) into a therapeutic population of TILs, the method comprising the steps of:
(a) obtaining and/or receiving a first population of TILs from surgical resection, needle biopsy, core biopsy, small biopsy, or other means for obtaining a sample that contains a mixture of tumor and TIL cells from a cancer in the subject or patient;
(b) contacting the first population of TILs with a first cell culture medium;
(c) performing an initial expansion (or priming first expansion) of the first population of TILs in the first cell culture medium to obtain a second population of TILs, wherein the first cell culture medium comprises IL-2, optionally OKT-3 (anti-CD3 antibody), and optionally antigen presenting cells (APCs), where the priming first expansion occurs for a period of 1 to 8 days;
(d) performing a rapid second expansion of the second population of TILs in a second cell culture medium to obtain a third population of TILs; wherein the second cell culture medium comprises IL-2, OKT-3 (anti-CD3 antibody), and APCs; and wherein the rapid expansion is performed over a period of 14 days or less, optionally the rapid second expansion can proceed for 1 day, 2 days, 3 days, 4, days, 5 days, 6 days, 7 days, 8 days, 9 days or 10 days after initiation of the rapid second expansion;
(e) harvesting the third population of TILs; and
(f) modifying a portion of the first, second, or third population of TILs at any time prior to or after the harvesting in step (f) to generate modified TILs each comprising an immunomodulatory composition associated with its surface membrane.

In one aspect, provided herein is a method of expanding tumor infiltrating lymphocytes (TILs) into a therapeutic population of TILs, the method comprising the steps of:
(a) resecting a tumor from a cancer in a subject or patient, the tumor comprising a first population of TILs, optionally from surgical resection, needle biopsy, core biopsy, small biopsy, or other means for obtaining a sample of the tumor that contains a mixture of tumor and TIL cells;
(b) fragmenting the tumor into tumor fragments;
(c) contacting the tumor fragments with a first cell culture medium;
(d) performing an initial expansion (or priming first expansion) of the first population of TILs in the first cell culture medium to obtain a second population of TILs, wherein the first cell culture medium comprises IL-2, optionally OKT-3 (anti-CD3 antibody), and optionally antigen presenting cells (APCs), where the priming first expansion occurs for a period of 1 to 8 days;
(e) performing a rapid second expansion of the second population of TILs in a second cell culture medium to obtain a third population of TILs; wherein the second cell culture medium comprises IL-2, OKT-3 (anti-CD3 antibody), and APCs; and wherein the rapid expansion is performed over a period of 14 days or less, optionally the rapid second expansion can proceed for 1 day, 2 days, 3 days, 4, days, 5 days, 6 days, 7 days, 8 days, 9 days or 10 days after initiation of the rapid second expansion;
(f) harvesting the third population of TILs; and
(g) modifying a portion of the first, second, or third population of TILs at any time prior to or after the harvesting in step (f) to generate modified TILs each comprising an immunomodulatory composition associated with its surface membrane.

In one aspect, provided herein is a method for expanding tumor infiltrating lymphocytes (TILs) into a therapeutic population of TILs comprising:
(a) obtaining and/or receiving a first population of TILs from a tumor resected from a cancer in a subject by processing a tumor sample obtained from the tumor into multiple tumor fragments;
(b) performing a priming first expansion by culturing the first population of TILs in a cell culture medium comprising IL-2, optionally OKT-3, and optionally comprising antigen presenting cells (APCs), to produce a second population of TILs, wherein the priming first expansion is performed for a first period of about 1 to 7/8 days to obtain the second population of TILs, wherein the second population of TILs is greater in number than the first population of TILs;
(c) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs;
(d) harvesting the therapeutic population of TILs obtained from step (c); and
(e) modifying a portion of the first, second, or third population of TILs at any time prior to or after the harvesting in step (d) to generate modified TILs each comprising an immunomodulatory composition associated with its surface membrane.

In some embodiments of this method, the cell culture medium in step (b) further comprises antigen-presenting cells (APCs), and wherein the number of APCs in the culture medium in step (c) is greater than the number of APCs in the culture medium in step (b).

In one aspect, provided herein is a method of expanding tumor infiltrating lymphocytes (TILs) into a therapeutic population of TILs comprising:
(a) performing a priming first expansion by culturing a first population of TILs, said first population of TILs obtainable by processing a tumor sample from a tumor resected from a cancer in a subject into multiple tumor fragments, in a cell culture medium comprising IL-2, optionally OKT-3, and optionally antigen presenting cells (APCs) to produce a second population of TILs, wherein the priming first expansion is performed in a container comprising a first gas-permeable surface area, wherein the priming first expansion is performed for first period of about 1 to 7/8 days to obtain the second population of TILs, wherein the second population of TILs is greater in number than the first population of TILs;
(b) performing a rapid second expansion by contacting the second population of TILs to a cell culture medium of the second population of TILs with additional IL-2, OKT-3, and APCs, to produce a third population of TILs, wherein the number of APCs in the rapid second expansion is at least twice the number of APCs in step (a), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area;
(c) harvesting the therapeutic population of TILs obtained from step (b); and
(d) modifying a portion of the first, second, or third population of TILs at any time prior to or after the harvesting in step (c) to generate modified TILs each comprising an immunomodulatory composition associated with its surface membrane.

In one aspect, provided herein is a method for expanding tumor infiltrating lymphocytes (TILs) into a therapeutic population of TILs comprising:
(a) performing a priming first expansion by culturing a first population of TILs in a cell culture medium comprising IL-2, optionally OKT-3, and optionally comprising antigen presenting cells (APCs), to produce a second population of TILs, wherein the priming first expansion is performed for a first period of about 1 to 7/8 days to obtain the second population of TILs, wherein the second population of TILs is greater in number than the first population of TILs;
(b) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs;
(c) harvesting the therapeutic population of TILs obtained from step (b); and
(d) modifying a portion of the first, second, or third population of TILs at any time prior to or after the harvesting in step (c) to generate modified TILs each comprising an immunomodulatory composition associated with its surface membrane.

In some embodiments of this method, the cell culture medium in step (a) further comprises antigen-presenting cells (APCs), and wherein the number of APCs in the culture medium in step (c) is greater than the number of APCs in the culture medium in step (b).

In some embodiments of the methods provided herein, the priming first expansion is divided into a first step and a second step, wherein the method further comprises performing the first step of the priming first expansion by culturing the first population of TILs in a cell culture medium containing IL-2 to produce TILs that egress from the tumor fragments or sample, separating TILs that remain in the tumor fragments or sample from TILs that egressed from the tumor fragments or sample, optionally digesting the tumor fragments or sample to produce a tumor digest, and performing the second step of the priming first expansion in the cell culture medium the TILs remaining in the tumor fragments or sample or tumor digest to produce the second population of TILs.

In one aspect, provided herein is a method for expanding tumor infiltrating lymphocytes (TILs) into a therapeutic population of TILs comprising:
(a) obtaining and/or receiving a first population of TILs from a tumor sample obtained from one or more small biopsies, core biopsies, or needle biopsies of a tumor from a cancer in a subject by culturing the tumor sample in a first cell culture medium comprising IL-2 for about 3 days;
(b) performing a priming first expansion by culturing the first population of TILs in a second cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 7 or 8 days to obtain the second population of TILs, wherein the second population of TILs is greater in number than the first population of TILs;
(c) performing a rapid second expansion by supplementing the second cell culture medium of the second population of TILs with additional IL-2, OKT-3, and APCs, to produce a third population of TILs, wherein the number of APCs added in the rapid second expansion is at least twice the number of APCs added in step b), wherein the rapid second expansion is performed for a second period of about 11 days to obtain the third population of TILs, wherein the third population of TILs is a therapeutic population of TILs, wherein the rapid second expansion is performed in a container comprising a second gas-permeable surface area;
(d) harvesting the therapeutic population of TILs obtained from step (c);
(e) transferring the harvested TIL population from step (d) to an infusion bag; and
(f) modifying a portion of the first, second, or third population of TILs at any time prior to transfer to the infusion bag in step (e) to generate modified TILs each comprising an immunomodulatory composition associated with its surface membrane.

In another aspect, provided herein is a method for expanding tumor infiltrating lymphocytes (TILs) into a therapeutic population of TILs comprising:
(a) obtaining and/or receiving a first population of TILs from a tumor sample obtained from one or more small biopsies, core biopsies, or needle biopsies of a tumor from a cancer in a subject by culturing the tumor sample in a first cell culture medium comprising IL-2 for about 3 days;
(b) performing a priming first expansion by culturing the first population of TILs in a second cell culture medium comprising IL-2, OKT-3, and antigen presenting cells (APCs) to produce a second population of TILs, wherein the priming first expansion is performed for first period of about 7 or 8 days to obtain the second population of TILs, wherein the second population of TILs is greater in number than the first population of TILs;
(c) performing a rapid second expansion by contacting the second population of TILs with a third cell culture medium comprising IL-2, OKT-3, and APCs, to produce a third population of TILs, wherein the rapid second expansion is performed for a second period of about 11 days to obtain the third population of TILs, wherein the third population of TILs is a therapeutic population of TILs;
(d) harvesting the therapeutic population of TILs obtained from step (c); and
(e) modifying a portion of the first, second, or third population of TILs at any time prior to or after the harvesting in step (f) to generate modified TILs each comprising an immunomodulatory composition associated with its surface membrane.

In some embodiments of the methods provided herein, the cancer is selected from the group consisting of melanoma, ovarian cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), renal cancer, and renal cell carcinoma.

In one aspect, provided herein is a method of expanding T cells comprising:
(a) performing a priming first expansion of a first population of T cells obtained from a donor by culturing the first population of T cells to effect growth and to prime an activation of the first population of T cells;
(b) after the activation of the first population of T cells primed in step (a) begins to decay, performing a rapid second expansion of the first population of T cells by culturing the first population of T cells to effect growth and to boost the activation of the first population of T cells to obtain a second population of T cells;
(c) harvesting the second population of T cells; and
(d) modifying a portion of the first or second population of T cells at any time prior to or after the harvesting in step (c) to generate modified T cells each comprising an immunomodulatory composition associated with its surface membrane.

In another aspect, provided herein is a method of expanding T cells comprising:
(a) performing a priming first expansion of a first population of T cells from a tumor sample obtained from one or more small biopsies, core biopsies, or needle biopsies of a tumor in a donor by culturing the first population of T cells to effect growth and to prime an activation of the first population of T cells;
(b) after the activation of the first population of T cells primed in step (a) begins to decay, performing a rapid second expansion of the first population of T cells by culturing the first population of T cells to effect growth and to boost the activation of the first population of T cells to obtain a second population of T cells;
(c) harvesting the second population of T cells; and
(d) modifying a portion of the first or second population of T cells at any time prior to or after the harvesting in step (e) to generate modified T cells each comprising an immunomodulatory composition associated with its surface membrane

In one aspect, provided herein is a method for expanding peripheral blood lymphocytes (PBLs) from peripheral blood, the method comprising the steps of:
(a) obtaining a sample of peripheral blood mononuclear cells (PBMCs) from peripheral blood of a patient;
(b) culturing said PBMCs in a culture comprising a first cell culture medium with IL-2, anti-CD3/anti-CD28 antibodies and a first combination of antibiotics, for a period of time selected from the group consisting of: about 9 days, about 10 days, about 11 days, about 12 days, about 13 days and about 14 days, thereby effecting expansion of peripheral blood lymphocytes (PBLs) from said PBMCs;
(c) harvesting the PBLs from the culture in step (b); and
(d) modifying a portion of the PBLs at any time prior to or after the harvesting in step (c) to generate modified PBLs each comprising an immunomodulatory composition associated with its surface membrane.

In some embodiments, the patient is pre-treated with ibrutinib or another interleukin-2 inducible T cell kinase (ITK) inhibitor. In certain embodiments, the patient is refractory to treatment with ibrutinib or another ITK inhibitor.

In some embodiments, the immunomodulatory composition comprises one or more membrane anchored immunomodulatory fusion proteins each comprising one or more immunomodulatory agents and a cell membrane anchor moiety.

In exemplary embodiments, the one or more immunomodulatory agents comprise one or more cytokines. In some embodiments, the one or more cytokines comprise one or more of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof.

In some embodiments, the one or more cytokines comprise IL-2 or a variant thereof. In some embodiments, the IL-2 is human IL-2. In exemplary embodiments, the human IL-2 has the amino acid sequence of SEQ ID NO:272.

In some embodiments, the one or more cytokines comprise one or more of IL-12 or a variant thereof. In certain embodiments, the IL-12 comprises a human IL-12 p35 subunit attached to a human IL-12 p40 subunit. In certain embodiments, the human IL-12 p35 subunit has the amino acid sequence of SEQ ID NO:267 and the human IL-12 p40 subunit has the amino acid sequence of SEQ ID NO:268.

In some embodiments, the one or more cytokines comprise IL-15 or a variant thereof. In some embodiments, the IL-15 is human IL-15. In exemplary embodiments, the human IL-15 has the amino acid sequence of SEQ ID NO:258.

In some embodiments, the one or more cytokines comprise IL-18 or a variant thereof. In certain embodiments, the IL-18 is human IL-18. In certain embodiments, the human IL-18 has the amino acid sequence of any one of SEQ ID NOs:269, 270, and 331-385.

In some embodiments, the one or more cytokines comprise IL-21 or a variant thereof. In certain embodiments, the IL-21 is human IL-21. In some embodiments, the human IL-21 has the amino acid sequence of SEQ ID NO:251.

In some embodiments, the one or more cytokines comprise IL-15 or a variant thereof and IL-21 or a variant thereof. In some embodiments, the IL-15 is human IL-15 and the IL-21 is human IL-21. In certain embodiments, the human IL-15 has the amino acid sequence of SEQ ID NO: 258 and the human IL-21 has the amino acid sequence of SEQ ID NO:271.

In some embodiments, the one or more immunomodulatory agents comprise a CD40 agonist. In certain embodiments, the CD40 agonist is an anti-CD40 binding domain or CD40L. In exemplary embodiments, the CD40 agonist is a CD40 binding domain comprising a variable heavy domain (VH) and a variable light domain (VL). In some embodiments, the VH and VL of the CD40 binding domain are selected from the following: a) a VH having the amino acid sequence of SEQ ID NO: 274, and a VL having the amino acid sequence of SEQ ID NO:275; b) a VH having the amino acid sequence of SEQ ID NO: 277, and a VL having the amino acid sequence of SEQ ID NO:278; c) a VH having the amino acid sequence of SEQ ID NO: 280, and a VL having the amino acid sequence of SEQ ID NO:281; and d) a VH having the amino acid sequence of SEQ ID NO: 283, and a VL having the amino acid sequence of SEQ ID NO:284. In exemplary embodiments, the CD40 binding domain is an scFv.

In some embodiments, the CD40 agonist is a human CD40L having the amino acid sequence of SEQ ID NO: 273.

In some embodiments, the one or more membrane anchored immunomodulatory fusion proteins are independently according to the formula, from N- to C-terminus: S-IA-L-C, wherein S is a signal peptide, IA is an immunomodulatory agent, L is a linker and C is a cell membrane anchor moiety.

In some embodiments, the cell membrane anchor moiety comprises a CD8a transmembrane-intracellular domain, a B7-1 transmembrane domain, a B7-2 transmembrane domain, or a CD8a transmembrane domain. In exemplary embodiments, the cell membrane anchor moiety comprises a B7-1 transmembrane domain. In some embodiments, the cell membrane anchor moiety has the amino acid sequence of SEQ ID NO:239.

In some embodiments, the immunomodulatory composition comprises two or more different membrane anchored immunomodulatory fusion proteins, wherein each of the different membrane anchored immunomodulatory fusion proteins each comprise a different immunomodulatory agent. In some embodiments, the different immunomodulatory agents are selected from: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof, and a CD40 agonist. In some embodiments, the different immunomodulatory agents are selected from: IL-12 and IL-15, IL-15 and IL-18, IL-15 and IL-21, CD40L and IL-15, IL-15 and IL-21, IL-2 and IL-12, and a variant thereof.

In some embodiments, the modified TILs comprise a first membrane anchored immunomodulatory fusion protein and a second membrane anchored immunomodulatory fusion protein.

In some embodiments, the first membrane anchored immunomodulatory fusion protein comprises IL-15 or a variant thereof and the second membrane anchored immunomodulatory fusion protein comprises IL-21 or a variant thereof.

In exemplary embodiments, the first membrane anchored immunomodulatory fusion protein and the second membrane anchored immunomodulatory fusion protein are expressed under the control of an NFAT promoter in the modified TILs.

In exemplary embodiments, the one or more membrane anchored immunomodulatory fusion proteins are independently according to the formula, from N- to C-terminus: S-IA-L-C, wherein S is a signal peptide, IA is an immunomodulatory agent, L is a linker and C is a cell membrane anchor moiety. In some embodiments, IA is a cytokine. In exemplary embodiments, IA is selected from the group consisting of: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof. In some embodiments, IA is IL-2 or a variant thereof. In certain embodiments, IA is IL-12 or a variant thereof. In some embodiments, IA is IL-15 or a variant thereof. In certain embodiments, IA is IL-18 or a variant thereof. In certain embodiments, IA is a DR-IL-18. In certain embodiments, IA is IL-21 or a variant thereof.

In exemplary the one or more membrane anchored immunomodulatory fusion proteins are independently according to the formula, from N- to C-terminus: S1-IA1-L1-C1-L2-S2-IA2-L3-C2, wherein S1 and S2 are each independently a signal peptide, IA1 and IA2 are each independently an immunomodulatory agent, L1-L3 are each independently a linker, and C1 and C2 are each independently a cell membrane anchor moiety. In some embodiments, S1 and S2 are the same. In certain embodiments, C1 and C2 are the same. In some embodiments, L2 is a cleavable linker. In exemplary embodiments, L2 is a furin cleavable linker. In some embodiments, IA1 and IA2 are each independently a cytokine.

In some embodiments, IA1 and IA2 are each independently selected from the group consisting of: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof. In some embodiments, IA1 and IA2 are each independently selected from the group consisting of IL-2 and IL-12, with the proviso that one of IA1 and IA2 is IL-2 and the other is IL-12. In some embodiments, IA1 and IA2 are each independently selected from the group consisting of IL-15 and IL-21, with the proviso that one of IA1 and IA2 is IL-15 and the other is IL-21.

In certain embodiments, the modifying comprises introducing a heterologous nucleic acid encoding the fusion protein into the portion of TILs and expressing the fusion protein on the surface of the modified TILs.

In certain embodiments, the modifying comprises introducing a heterologous nucleic acid encoding the fusion protein into the portion of TILs and expressing the fusion protein on the surface of the modified TILs. In some embodiments, the heterologous nucleic acid comprises a viral vector (e.g., an adenoviral vector, a retroviral vector, a lentiviral vector, or an adeno-associated vector (AAV)). In some embodiments, the heterologous nucleic acid comprises a piggyBac transposon. In some embodiments, the heterologous nucleic acid comprises an NFAT promoter, an EF-1a promoter, an MND promoter, or an SSFV promoter.

In some embodiments, the immunomodulatory composition comprises a fusion protein comprising one or more immunomodulatory agents linked to a TIL surface antigen binding domain. In some embodiments, the one or more immunomodulatory agents comprise one or more cytokines. In some embodiments, the one or more cytokines comprise one or more of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof. In some embodiments, the one or more cytokines comprise IL-12 or a variant thereof. In certain embodiments, the one or more cytokines comprise IL-15 or a variant thereof. In certain embodiments, the one or more cytokines comprise IL-18 or a variant thereof (e.g., a DR-IL-18). In some embodiments, the one or more cytokines comprise IL-21 or a variant thereof. In certain embodiments, the TIL surface antigen binding domain comprises an antibody variable heavy domain and variable light domain. In some embodiments, the TIL surface antigen binding domain comprises an antibody or fragment thereof. In some embodiments, the TIL surface antigen binding domain exhibits an affinity for one or more of following TIL surface antigens: CD45, CD4, CD8, CD3, CDlla, CDllb, CDllc, CD18, CD25, CD127, CD19, CD20, CD22, HLA-DR, CD197, CD38, CD27, CD196, CXCR3, CXCR4, CXCR5, CD84, CD229, CCR1, CCR5, CCR4, CCR6, CCR8, CCR10, CD 16, CD56, CD 137, OX40, or GITR. In certain embodiments, the modifying comprises incubating the fusion protein with the portion of TILs under conditions to permit the binding of the fusion protein to the portion of TILs.

In some embodiments, the immunomodulatory composition comprises a nanoparticle comprising a plurality of immunomodulatory agents. In some embodiments, the plurality of immunomodulatory agents are covalently linked together by degradable linkers. IN certain embodiments, the nanoparticle comprises at least one polymer, cationic polymer, or cationic block co-polymer on the nanoparticle surface. In some embodiments, the one or more cytokines comprise one or more of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof. In certain embodiments, the one or more cytokines comprises IL-12. In some embodiments, the one or more cytokines comprises IL-15. In some embodiments, the one or more cytokines comprise IL-21. In some embodiments, the nanoparticle is a liposome, a protein nanogel, a nucleotide nanogel, a polymer nanoparticle, or a solid nanoparticle. In some embodiments, the nanoparticle is a nanogel. In certain embodiments, the nanoparticle further comprises an antigen binding domain that binds to one or more of the following antigens: CD45, CDlla (integrin alpha-L), CD 18 (integrin beta-2), CD1lb, CD1lc, CD25, CD8, or CD4. In some embodiments, the modifying comprises attaching the immunomodulatory composition to the surface of the portion of TILs.

In certain embodiments of the methods provided herein, the modifying is carried out on TILs from the first expansion, or TILs from the second expansion, or both. In certain embodiments, the modifying is carried out on TILs from the priming first expansion, or TILs from the rapid second expansion, or both.

In some embodiments of the methods provided herein, the modifying is carried out after the first expansion and before the second expansion. In some embodiments, the modifying is carried out after the priming first expansion and before the rapid second expansion, or both. In certain embodiments, the modifying is carried out after the second expansion. In some embodiments, the modifying is carried out after the rapid second expansion. In some embodiments, the modifying is carried out after the harvesting.

In certain embodiments, the first expansion is performed over a period of about 11 days. In some embodiments, the priming first expansion is performed over a period of about 11 days.

In some embodiments of the methods provided herein, the IL-2 is present at an initial concentration of between 1000 IU/mL and 6000 IU/mL in the cell culture medium in the first expansion. In certain embodiments, the IL-2 is present at an initial concentration of between 1000 IU/mL and 6000 IU/mL in the cell culture medium in the priming first expansion.

In some embodiments, the IL-2 in the second expansion step is present at an initial concentration of between 1000 IU/mL and 6000 IU/mL and the OKT-3 antibody is present at an initial concentration of about 30 ng/mL. In some embodiments, the IL-2 in the rapid second expansion step is present at an initial concentration of between 1000 IU/mL and 6000 IU/mL and the OKT-3 antibody is present at an initial concentration of about 30 ng/mL.

In some embodiments, the first expansion is performed using a gas permeable container. In certain embodiments, the priming first expansion is performed using a gas permeable container. In some embodiments, the second expansion is performed using a gas permeable container. In certain embodiments, the rapid second expansion is performed using a gas permeable container.

In some embodiments of the methods provided herein, the cell culture medium of the first expansion further comprises a cytokine selected from the group consisting of IL-4, IL-7, IL-15, IL-21, and combinations thereof. In certain embodiments, the cell culture medium of the priming first expansion further comprises a cytokine selected from the group consisting of IL-4, IL-7, IL-15, IL-21, and combinations thereof. In some embodiments, the cell culture medium of the second expansion further comprises a cytokine selected from the group consisting of IL-4, IL-7, IL-15, IL-21, and combinations thereof. In certain embodiments, the cell culture medium of the rapid second expansion further comprises a cytokine selected from the group consisting of IL-4, IL-7, IL-15, IL-21, and combinations thereof.

In some embodiments of the methods of treatment provided herein, the method further includes the step of treating the patient with a non-myeloablative lymphodepletion regimen prior to administering the TILs to the patient. In some embodiments, the non-myeloablative lymphodepletion regimen comprises the steps of administration of cyclophosphamide at a dose of 60 mg/m²/day for two days followed by administration of fludarabine at a dose of 25 mg/m²/day for three days. In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days. In certain embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for one day. In certain embodiments, the cyclophosphamide is administered with mesna.

In some embodiments of the methods of treatment provided herein, the method further includes the step of treating the patient with an IL-2 regimen starting on the day after the administration of TILs to the patient. In some embodiments of the methods of treatment provided herein, the method further includes the step of treating the patient with an IL-2 regimen starting on the same day as administration of TILs to the patient. In some embodiments, the IL-2 regimen is a high-dose IL-2 regimen comprising 600,000 or 720,000 IU/kg of aldesleukin, or a biosimilar or variant thereof, administered as a 15-minute bolus intravenous infusion every eight hours until tolerance.

In some embodiments of the methods provided herein, the therapeutically effective population of TILs is administered and comprises from about 2.3×10¹⁰ to about 13.7×10¹⁰ TILs.

In some embodiments of the methods provided herein, the priming first expansion and rapid second expansion are performed over a period of 21 days or less. In some embodiments, the priming first expansion and rapid second expansion are performed over a period of 16 or 17 days or less. In certain embodiments, the priming first expansion is performed over a period of 7 or 8 days or less. In some embodiments, the rapid second expansion is performed over a period of 11 days or less.

In some embodiments, of the methods provided herein the first expansion in step (c) and the second expansion in step (d) are each individually performed within a period of 11 days. In some embodiments of the methods provided herein, steps (a) through (f) are performed in about 10 days to about 22 days.

In some embodiments of the methods provided herein, the modified TILs further comprise a genetic modification that causes expression of one or more immune checkpoint genes to be silenced or reduced in at least a portion of the therapeutic population of TILs. IN some embodiments, the one or more immune checkpoint genes is/are selected from the group comprising PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, and BCOR. In certain embodiments, the one or more immune checkpoint genes is/are selected from the group comprising PD-1, TGIT, CTLA-4, LAG-3, HAVCR2 (TIM-3), Cish, TGFβ, and PKA. In certain embodiments, the immune checkpoint gene is PD-1. In some embodiments, the genetic modification is produced using an RNA interference method (e.g., shRNA).

In some embodiments, the modified TILs further comprises a genetic modification that causes expression of one or more immune checkpoint genes to be enhanced in at least a portion of the therapeutic population of TILs, the immune checkpoint gene(s) being selected from the group comprising CCR2, CCR4, CCR5, CXCR2, CXCR3, CX3CR1, IL-2, IL-4, IL-7, IL-10, IL-15, IL-21, the NOTCH 1/2 intracellular domain (ICD), and/or the NOTCH ligand mDLL1. In certain embodiments, the genetic modification is produced using a programmable nuclease that mediates the generation of a double-strand or single-strand break at said one or more immune checkpoint genes. In some embodiments, the genetic modification is produced using one or more methods selected from an RNA interference method (e.g., shRNA), a CRISPR method, a TALE method, a zinc finger method, a Cas-CLOVER method, and a combination thereof. In certain embodiments, the genetic modification is produced using a CRISPR method. In some embodiments, the CRISPR method is a CRISPR/Cas9 method. In certain embodiments, the genetic modification is produced using a TALE method. In some embodiments, the genetic modification is produced using a zinc finger method. In some embodiments, the genetic modification is produced using a Cas-CLOVER method.

In some embodiments, the modified TILs transiently express the immunomodulatory composition on the cell surface. In some embodiments, the immunomodulatory composition comprises one or more membrane anchored immunomodulatory fusion proteins, wherein each fusion protein comprises one or more immunomodulatory agents and a cell membrane anchor moiety.

In exemplary embodiments, the one or more immunomodulatory agents comprise one or more cytokines. In some embodiments, the one or more cytokines comprise one or more of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof.

In some embodiments, the one or more cytokines comprise IL-2 or a variant thereof. In some embodiments, the IL-2 is human IL-2. In exemplary embodiments, the human IL-2 has the amino acid sequence of SEQ ID NO:272.

In some embodiments, the one or more cytokines comprise IL-12 or a variant thereof. In certain embodiments, the IL-12 comprises a human IL-12 p35 subunit attached to a human IL-12 p40 subunit. In certain embodiments, the human IL-12 p35 subunit has the amino acid sequence of SEQ ID NO:267 and the human IL-12 p40 subunit has the amino acid sequence of SEQ ID NO:268.

In some embodiments, the one or more cytokines comprise IL-15 or a variant thereof. In some embodiments, the IL-15 is human IL-15. In exemplary embodiments, the human IL-15 has the amino acid sequence of SEQ ID NO:258.

In some embodiments, the one or more cytokines comprise IL-18 or a variant thereof (e.g., DR-IL-18). In certain embodiments, the IL-18 is human IL-18. In certain embodiments, the human IL-18 has the amino acid sequence of any one of SEQ ID NOs:269, 270, and 331-385.

In some embodiments, the one or more cytokines comprise IL-21 or a variant thereof. In certain embodiments, the IL-21 is human IL-21. In some embodiments, the human IL-21 has the amino acid sequence of SEQ ID NO:271.

In some embodiments, the one or more cytokines comprise IL-15 and IL-21. In some embodiments, the IL-15 is human IL-15 and the IL-21 is human IL-21. In certain embodiments, the human IL-15 has the amino acid sequence of SEQ ID NO: 258 and the human IL-21 has the amino acid sequence of SEQ ID NO:271.

In some embodiments, the one or more immunomodulatory agents comprise a CD40 agonist. In certain embodiments, the CD40 agonist is an anti-CD40 binding domain or CD40L. In exemplary embodiments, the CD40 agonist is a CD40 binding domain comprising a variable heavy domain (VH) and a variable light domain (VL). In some embodiments, the VH and VL of the CD40 binding domain are selected from the following: a) a VH having the amino acid sequence of SEQ ID NO: 274, and a VL having the amino acid sequence of SEQ ID NO:275; b) a VH having the amino acid sequence of SEQ ID NO: 277, and a VL having the amino acid sequence of SEQ ID NO:278; c) a VH having the amino acid sequence of SEQ ID NO: 280, and a VL having the amino acid sequence of SEQ ID NO:281; and d) a VH having the amino acid sequence of SEQ ID NO: 283, and a VL having the amino acid sequence of SEQ ID NO:284. In exemplary embodiments, the CD40 binding domain is an scFv.

In some embodiments, the CD40 agonist is a human CD40L having the amino acid sequence of SEQ ID NO: 273. In some embodiments, the membrane anchored immunomodulatory fusion protein is according to the formula, from N- to C-terminus: S-IA-L-C, wherein S is a signal peptide, IA is an immunomodulatory agent, L is a linker and C is a cell membrane anchor moiety.

In some embodiments, the cell membrane anchor moiety comprises a CD8a transmembrane-intracellular domain, a B7-1 transmembrane domain, a B7-2 transmembrane domain, or a CD8a transmembrane domain. In exemplary embodiments, the cell membrane anchor moiety comprises a B7-1 transmembrane domain. In some embodiments, the cell membrane anchor moiety has the amino acid sequence of SEQ ID NO:239.

In some embodiments, the immunomodulatory composition comprises two or more different membrane anchored immunomodulatory fusion proteins, wherein each of the different membrane anchored immunomodulatory fusion proteins each comprise a different immunomodulatory agent. In some embodiments, the different immunomodulatory agents are selected from: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof, and a CD40 agonist. In some embodiments, the different immunomodulatory agents are selected from: IL-12 and IL-15, IL-15 and IL-18, CD40L, IL-15 and IL-21, and IL-15, and IL-2 and IL-12.

In some embodiments, the modified TILs are modified by transfecting the TILs with a nucleic acid encoding a fusion protein comprising one or more immunomodulatory agents and a cell membrane anchor moiety in order to transiently express the fusion protein on the cell surface. In some embodiments, the nucleic acid is an RNA. In some embodiments, the RNA is a mRNA. In some embodiments, the TILs are transfected with the mRNA by electroporation. In some embodiments, the TILs are transfected with the mRNA by electroporation after the first expansion and before the second expansion. In some embodiments, the TILs are transfected with the mRNA by electroporation before the first expansion. In some embodiments, the method further comprises activating the TILs by incubation with an anti-CD3 agonist before transfecting the TILs with the mRNA. In some embodiments, the anti-CD3 agonist is OKT-3. In some embodiments, the TILs are activated by incubating the TILs with the anti-CD3 agonist for about 1 to 3 days before transfecting the TILs with the mRNA.

In some embodiments, the modified TILs are transfected with the nucleic acid encoding the fusion protein using a microfluidic device to temporarily disrupt the cell membranes of the TILs, thereby allowing transfection of the nucleic acid.

In some embodiments, artificial antigen-presenting cells (aAPCs) are used in place of APCs. In some embodiments, the aAPCs comprise a cell that expresses HLA-A/B/C, CD64, CD80, ICOS-L, and CD58. In some embodiments, the aAPCs comprise a MOLM-14 cell. In some embodiments, the aAPCs comprise a MOLM-13 cell. In some embodiments, the aAPCs comprise a MOLM-14 cell that endogenously expresses HLA-A/B/C, CD64, CD80, ICOS-L, and CD58. In some embodiments, the aAPCs comprise a MOLM-14 cell that endogenously expresses HLA-A/B/C, CD64, CD80, ICOS-L, and CD58, wherein the MOLM-14 cell is permanently gene-edited to express CD86. In some embodiments, the MOLM-14 cell transduced with one or more viral vectors, wherein the one or more viral vectors comprise a nucleic acid sequence encoding CD86 and a nucleic acid sequence encoding 4-1BBL, and wherein the MOLM-14 cell expresses CD86 and 4-1BBL. In some embodiments, the aAPCs are transiently gene-edited to transiently express on the cell surface an immunomodulatory composition comprising an immunomodulatory fusion protein. In some embodiments, the aAPCs transiently express on the cell surface an immunomodulatory fusion protein comprising a membrane anchor fused to a cytokine. In some embodiments, the aAPCs transiently express on the cell surface a membrane anchor fused to a cytokine selected from the group consisting of IL-2, IL-7, IL-10, IL-12, IL-15, and IL-21. In some embodiments, the aAPCs transiently express on the cell surface a membrane anchor fused to a cytokine selected from the group consisting of IL-2, IL-12, IL-15, and IL-21. In some embodiments, the aAPCs transiently express on the cell surface a membrane anchor fused to a cytokine selected from the group consisting of IL-12, IL-15, and IL-21.

In some embodiments, the modified TILs are genetically modified to express the immunomodulatory composition on the cell surface. In some embodiments, the immunomodulatory composition comprises one or more membrane anchored immunomodulatory fusion proteins each comprising one or more immunomodulatory agents and a cell membrane anchor moiety. In some embodiments, the one or more membrane anchored immunomodulatory fusion proteins comprise IL-2 or a variant thereof. In certain embodiments, the one or more membrane anchored immunomodulatory fusion proteins comprise IL-15 or a variant thereof. In exemplary embodiments, the one or more membrane anchored immunomodulatory fusion proteins comprise IL-18 or a variant thereof (e.g., a DR-IL-18). In some embodiments, the one or more membrane anchored immunomodulatory fusion proteins comprise IL-21 or a variant thereof.

In certain embodiments, the modified TILs comprise a first membrane anchored immunomodulatory fusion protein and a second membrane anchored immunomodulatory fusion protein. In some embodiments, the first membrane anchored immunomodulatory fusion protein comprises IL-15 and the second membrane anchored immunomodulatory fusion protein comprises IL-21. In some embodiments, the first membrane anchored immunomodulatory fusion protein and the second immunomodulatory fusion protein are expressed under the control of an NFAT promoter, an EF-1a promoter, an MND promoter, or an SSFV promoter in the modified TILs.

In some embodiments, the one or more membrane anchored immunomodulatory fusion proteins are independently according to the formula, from N- to C-terminus: S-IA-L-C, wherein S is a signal peptide, IA is an immunomodulatory agent, L is a linker and C is a cell membrane anchor moiety. In some embodiments, IA is a cytokine. In exemplary embodiments, IA is selected from the group consisting of: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof. In some embodiments, IA is IL-2. In certain embodiments, IA is IL-12. In some embodiments, IA is IL-15. In certain embodiments, IA is IL-21. In some embodiments, L is a CD8a transmembrane-intracellular domain, a B7-1 transmembrane domain, a B7-2 transmembrane domain, or a CD8a transmembrane domain. In certain embodiments, L is a B7-1 transmembrane domain. In some embodiments, L has the amino acid sequence of SEQ ID NO:239.

In exemplary embodiments, the one or more membrane anchored immunomodulatory fusion proteins are independently according to the formula, from N- to C-terminus: S1-IA1-L1-C1-L2-S2-IA2-L3-C2, wherein S1 and S2 are each independently a signal peptide, IA1 and IA2 are each independently an immunomodulatory agent, L1-L3 are each independently a linker, and C1 and C2 are each independently a cell membrane anchor moiety. In some embodiments, S1 and S2 are the same. In exemplary embodiments, C1 and C2 are the same. In some embodiments, L2 is a cleavable linker. In certain embodiments, L2 is a furin cleavable linker.

In some embodiments, IA1 and IA2 are each independently a cytokine. In some embodiments, IA1 and IA2 are each independently selected from the group consisting of: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof. In some embodiments, IA1 and IA2 are each independently selected from the group consisting of IL-2 and IL-12, with the proviso that one of IA1 and IA2 is IL-2 and the other is IL-12. In some embodiments, IA1 and IA2 are each independently selected from the group consisting of IL-15 and IL-21, with the proviso that one of IA1 and IA2 is IL-15 and the other is IL-21.

In exemplary embodiments, C1 and C2 are each independently a CD8a transmembrane-intracellular domain, a B7-1 transmembrane domain, a B7-2 transmembrane domain, or a CD8a transmembrane domain. In some embodiments, C1 and C2 are each a B7-1 transmembrane domain. In some embodiments, C1 and C2 each have the amino acid sequence of SEQ ID NO:239.

In certain embodiments, the modified TILs express the one or more membrane anchored immunomodulatory fusion proteins under the control of an NFAT promoter, an EF-1a promoter, an MND promoter, or an SSFV promoter. In some embodiments, the modified TILs are transduced with a retroviral vector to express the one or more membrane anchored immunomodulatory fusion proteins. In some embodiments, the modified TILs are transduced with a vector (e.g., an adenoviral vector, a retroviral vector, a lentiviral vector, or an adeno-associated vector (AAV)) or a piggyBac transposon, to express the one or more membrane anchored immunomodulatory fusion proteins.

Also provided are composition that include the modified TILs produced by any of the methods described herein.

### BRIEF DESCRIPTION OF THE DRAWINGS

FIG. 1: Exemplary Gen 2 (process 2A) chart providing an overview of Steps A through F.

FIG. 2A-2C: Process flow chart of an embodiment of Gen 2 (process 2A) for TIL manufacturing.

FIG. 3: Shows a diagram of an embodiment of a cryopreserved TIL exemplary manufacturing process (˜22 days).

FIG. 4: Shows a diagram of an embodiment of Gen 2 (process 2A), a 22-day process for TIL manufacturing.

FIG. 5: Comparison table of Steps A through F from exemplary embodiments of process 1C and Gen 2 (process 2A) for TIL manufacturing.

FIG. 6: Detailed comparison of an embodiment of process 1C and an embodiment of Gen 2 (process 2A) for TIL manufacturing.

FIG. 7: Exemplary Gen 3 type TIL manufacturing process.

FIG. 8A-8D: A) Shows a comparison between the 2A process (approximately 22-day process) and an embodiment of the Gen 3 process for TIL manufacturing (approximately 14-days to 16-days process). B) Exemplary Process Gen 3 chart providing an overview of Steps A through F (approximately 14-days to 16-days process). C) Chart providing three exemplary Gen 3 processes with an overview of Steps A through F (approximately 14-days to 16-days process) for each of the three process variations. D) Exemplary modified Gen 2-like process providing an overview of Steps A through F (approximately 22-days process).

FIG. 9: Provides an experimental flow chart for comparability between Gen 2 (process 2A) versus Gen 3 processes.

FIG. 10: Shows a comparison between various Gen 2 (process 2A) and the Gen 3.1 process embodiment.

FIG. 11: Table describing various features of embodiments of the Gen 2, Gen 2.1 and Gen 3.0 process.

FIG. 12: Overview of the media conditions for an embodiment of the Gen 3 process, referred to as Gen 3.1.

FIG. 13: Table describing various features of embodiments of the Gen 2, Gen 2.1 and Gen 3.0 process.

FIG. 14: Table comparing various features of embodiments of the Gen 2 and Gen 3.0 processes.

FIG. 15: Table providing media uses in the various embodiments of the described expansion processes.

FIG. 16: Schematic of an exemplary embodiment of the Gen 3 process (a 16-day process).

FIG. 17: Schematic of an exemplary embodiment of a method for expanding T cells from hematopoietic malignancies using Gen 3 expansion platform.

FIG. 18: Provides the structures I-A and I-B. The cylinders refer to individual polypeptide binding domains. Structures I-A and I-B comprise three linearly-linked TNFRSF binding domains derived from e.g., 4-1BBL or an antibody that binds 4-1BB, which fold to form a trivalent protein, which is then linked to a second trivalent protein through IgG1-Fc (including CH3 and CH2 domains) is then used to link two of the trivalent proteins together through disulfide bonds (small elongated ovals), stabilizing the structure and providing an agonists capable of bringing together the intracellular signaling domains of the six receptors and signaling proteins to form a signaling complex. The TNFRSF binding domains denoted as cylinders may be scFv domains comprising, e.g., a VH and a VL chain connected by a linker that may comprise hydrophilic residues and Gly and Ser sequences for flexibility, as well as Glu and Lys for solubility.

FIG. 19: Schematic of an exemplary embodiment of the Gen 3 process (a 16-day process).

FIG. 20: Provides a process overview for an exemplary embodiment of the Gen 3.1 process (a 16 day process).

FIG. 21: Schematic of an exemplary embodiment of the Gen 3.1 Test process (a 16-17 day process).

FIG. 22: Schematic of an exemplary embodiment of the Gen 3 process (a 16-day process).

FIG. 23A-23B: Comparison table for exemplary Gen 2 and exemplary Gen 3 processes.

FIG. 24: Schematic of an exemplary embodiment of the Gen 3 process (a 16-17 day process) preparation timeline.

FIG. 25: Schematic of an exemplary embodiment of the Gen 3 process (a 14-16 day process).

FIG. 26A-26B: Schematic of an exemplary embodiment of the Gen 3 process (a 16 day process).

FIG. 27: Schematic of an exemplary embodiment of the Gen 3 process (a 16 day process).

FIG. 28: Comparison of Gen 2, Gen 2.1 and an embodiment of the Gen 3 process (a 16 day process).

FIG. 29: Comparison of Gen 2, Gen 2.1 and an embodiment of the Gen 3 process (a 16 day process).

FIG. 30: Gen 3 embodiment components.

FIG. 31: Gen 3 embodiment flow chart comparison (Gen 3.0, Gen 3.1 control, Gen 3.1 test).

FIG. 32: Shown are the components of an exemplary embodiment of the Gen 3 process (a 16-17 day process).

FIG. 33: Acceptance criteria table.

FIG. 34: Depiction of some embodiments of a TIL manufacturing process including electroporation step for use with gene-editing processes (including TALEN, zinc finger nuclease, and CRISPR methods as described herein).

FIG. 35: Depiction of embodiments of TIL manufacturing processes including electroporation step for use with gene-editing processes (including TALEN, zinc finger nuclease, and CRISPR methods as described herein).

FIG. 36A-36J: Exemplary membrane anchored immunomodulatory fusion proteins that can be included in the TILs described herein.

FIG. 37A-37D: Exemplary membrane anchored immunomodulatory fusion proteins that can be included in the TILs described herein.

FIG. 38A-38C: Summary of study to assess expression and signaling of membrane bound IL-15/IL-21 transduced pre-REP TILs.

FIG. 39A-39B: Summary of study to assess expression of mIL-15/IL21 and CD8 and CD4 T cell subset in mIL-15/IL-21 transduced REP TILs.

FIG. 40A-40C: Summary of study to assess phenotype of mIL-15/IL-21 transduced CD8+ REP TILs.

FIG. 41A-41C: Summary of study to assess phenotype of mIL-15/IL-21 transduced CD4+.

FIG. 42: Summary of study to assess fold expansion, cell viability, and transduction efficient of TeIL-18 and TeDR-IL18 expressing TILs after gene transduction and 11-day REP process described herein.

FIGS. 43A-43B: Summary of studies to assess surface expression of IL-18 and DRIL-18 on TeIL-18 and TeDR-IL18 expressing TILs.

FIG. 44: Summary of study to assess IFN-γ production by TeIL-18 and TeDR-IL18 expressing TILs with and without TCR stimulation using anti-CD3 antibody OKT3

FIG. 45: Summary of study to assess expression and IL-18 activity of TeIL-18 and TeDR-IL18 expressing REP TILs, wherein TeIL-18 and TeDRIL-18 are under the control of an inducible NFAT promoter.

FIG. 46A-46C: Summary of TeIL-IL18 and TeDRIL-18 REP TIL functional study using a KILR-THP-I cytotoxicity assay. (A)-(C) freshly thawed TILs. (B) and (C) includes additional experiments with repeated stimulated TILs.

FIG. 47A-47B: Summary of assessment o IFN-γ production by TeIL-18 and TeDRIL-18 ub KILR-THP-I cytotoxicity assay.

FIG. 48A-48H: Summary of phenotypic and functional analysis of TeIL-18 and TeDRIL-18 REP TILs. (A) and (B): Assessment of differentiation in freshly thawed CD4+ (A) and CD8+ (B) TeIL-18 and TeDRIL-18 REP TILs. (C) and (D): Assessment of differentiation in repeat stimulated CD4+(C) and CD8+(D) TeIL-18 and TeDRIL-18 REP TILs. (E) and (F): Assessment of activation in freshly thawed (E) and repeat stimulated (F) TeIL-18 and TeDRIL-18 REP TILs. (F) and (G): Assessment of activation in repeat stimulated CD4+ and CD8+ TeIL-18 and TeDRIL-18 REP TILs.

FIG. 49A-49B: Summary of study to assess the effects of TeIL-18 and TeDRIL-18 on THP-I MHC-I and -II expression.

FIG. 50 depicts an exemplary nucleic acid that allows for expression of a member anchored IL-12 (TeIL-12) and PD-1 shRNA in embodiments of the subject TILs provided herein.

FIG. 51 depicts an exemplary workflow for the preparation of TILs expressing TeIL-12 and/or NFAT-TeIL-12 for administration to a subject.

FIG. 52A-52B: Summary of study to assess the expression of TeIL-12 and/or NFAT-TeIL-12 in REP TILs. (A) After REP harvest, surface expression of TeIL-12 on TeIL12 TIL was examined by flow assay with IL-12P70 flow Ab (B). NFAT-TeIL-12 transduced REP-TIL was stimulated with TransACT with indicated dilution or PMA. 48 hours after stimulation, surface expression TeIL-12 expression was examined.

FIG. 53: Summary of a study to assess IL-12 activity in TeIL-12 expressing TILs.

FIG. 54A-54B: Summary of study to assess (A) expansion and (B) viability of post-REP TILs transduced to express TeIL-12 or NFAT-TeIL-12.

FIG. 55A-55B: Summary of study to assess (A) the frequency of TeIL-12 and/or NFAT-TeIL-12 in a population of REP TILs from various tissues (including two lung, one head & neck, one breast and one ovarian tumor samples) and (B) viral genome copy number (VCN) per cell.

FIG. 56A-56D: Summary of study to assess (A) and (B) cytotoxicity in a THP-1 based allogenic cytotoxicity assay, (C) and (D) IFN-γ production of TeIL-12 REP-TILs and NFAT-driven inducible TeIL-12 REP-TILs.

FIG. 57A-57B: Summary of study to assess cytotoxicity of TeIL-12 expressing TILs was also assessed by xCelligence RTCA assay using two target cell populations (A) and (B).

FIG. 58A-58C: Summary of a study to assess TIL killing efficacy. (A) Schematic of experimental design. (B) KILR® THP-1 cytotoxicity assay and IFN-g quantification, and (C) Xcellgene RTCA killing assay were performed.

FIG. 59 depicts a summary of study to assess distribution of CD8+, CD4+, and CD4+/FoxP3− T cells within a population of REP TILs transduced to express TeIL-12 or NFAT-TeIL-12.

FIG. 60A-60B: Summary of study to assess T cell differentiation of (A) CD8+ and (B) CD4+ T cells, as measured by various cellular markers, within a population of REP TILs transduced to express TeIL-12 or NFAT-TeIL-12.

FIG. 61A-61B: Summary of study to assess T cell exhaustion of (A) CD8+ and (B) CD4+ T cells, as measured by various cellular markers, within a population of REP TILs transduced to express TeIL-12 or NFAT-TeIL-12.

FIG. 62A-62B: Summary of study to assess T cell activation of (A) CD8+ and (B) CD4+ T cells, as measured by various cellular markers, within a population of REP TILs transduced to express TeIL-12 or NFAT-TeIL-12.

FIG. 63A-63B: Summary of study to assess T cell function of (A) CD8+ and (B) CD4+ T cells, as measured by various cellular markers, within a population of REP TILs transduced to express TeIL-12 or NFAT-TeIL-12.

FIG. 64A-64B: Shows (A) cell expansion and (B) surface expression of TeIL-15 after the procedure: after gene transduction of TeIL-15 lentivirus, Pre-REP TIL was processed for REP expansion with feeder cell, 3000 IU/ml IL-2 and aCD3 Ab OKT3 or HIT3a. OKT3 (30 ng/ml) or HIT3a(30 ng/ml) was added into REP culture medium in different days (Day 0, Day 2 and Day 4) after setting REP process. After 11 days REP expansion, Post-REP-TIL was harvested and analyzed.

FIG. 65A-65B: Shows (A) cell expansion and (B) surface expression of TeIL-15/TeIL-21 after the procedure: after gene transduction of TeIL-15/TeIL-21 lentivirus, Pre-REP TIL was processed for REP expansion with feeder cell, 3000 IU/ml IL-2 and aCD3 Ab OKT3 or HIT3a. OKT3 (30 ng/ml) or HIT3a (30 ng/ml) was added into REP culture medium in different days (Day 0, Day 2 and Day 4) after setting REP process. After 11 days REP expansion, Post-REP-TIL was harvested and analyzed.

FIG. 66A-66B: Shows (A) cell expansion and (B) surface expression of TeIL-15 after the procedure: after gene transduction of TeIL-15 lentivirus, Pre-REP TIL was processed for 11 days REP expansion with feeder cell, 3000 IU/ml IL-2 and OKT3 with indicated concentration. After 11 days REP expansion, Post-REP-TIL was harvested and analyzed.

FIG. 67A-67B: Shows (A) cell expansion and (B) surface expression of TeIL-15/TeIL-21 after the procedure: after gene transduction of TeIL-15/TeIL-21 lentivirus, Pre-REP TIL was processed for REP expansion with feeder cell, 3000 IU/ml IL-2 and OKT3 with indicated concentration. After 11 days REP expansion, Post-REP-TIL was harvested and analyzed.

### BRIEF DESCRIPTION OF THE SEQUENCE LISTING

SEQ ID NO:1 is the amino acid sequence of the heavy chain of muromonab.

SEQ ID NO:2 is the amino acid sequence of the light chain of muromonab.

SEQ ID NO:3 is the amino acid sequence of a recombinant human IL-2 protein.

SEQ ID NO:4 is the amino acid sequence of aldesleukin.

SEQ ID NO:5 is an IL-2 form.

SEQ ID NO:6 is the amino acid sequence of nemvaleukin alfa.

SEQ ID NO:7 is an IL-2 form.

SEQ ID NO:8 is a mucin domain polypeptide.

SEQ ID NO:9 is the amino acid sequence of a recombinant human IL-4 protein.

SEQ ID NO: 10 is the amino acid sequence of a recombinant human IL-7 protein.

SEQ ID NO:11 is the amino acid sequence of a recombinant human IL-15 protein.

SEQ ID NO: 12 is the amino acid sequence of a recombinant human IL-21 protein.

SEQ ID NO: 13 is an IL-2 sequence.

SEQ ID NO: 14 is an IL-2 mutein sequence.

SEQ ID NO: 15 is an IL-2 mutein sequence.

SEQ ID NO: 16 is the HCDR1_IL-2 for IgG.IL2R67A.H1.

SEQ ID NO: 17 is the HCDR2 for IgG.IL2R67A.H1.

SEQ ID NO: 18 is the HCDR3 for IgG.IL2R67A.H1.

SEQ ID NO: 19 is the HCDR1_IL-2 kabat for IgG.IL2R67A.H1.

SEQ ID NO:20 is the HCDR2 kabat for IgG.IL2R67A.H1.

SEQ ID NO:21 is the HCDR3 kabat for IgG.IL2R67A.H1.

SEQ ID NO:22 is the HCDR1_IL-2 clothia for IgG.IL2R67A.H1.

SEQ ID NO:23 is the HCDR2 clothia for IgG.IL2R67A.H1.

SEQ ID NO:24 is the HCDR3 clothia for IgG.IL2R67A.H1.

SEQ ID NO:25 is the HCDR1_IL-2 IMGT for IgG.IL2R67A.H1.

SEQ ID NO:26 is the HCDR2 IMGT for IgG.IL2R67A.H1.

SEQ ID NO:27 is the HCDR3 IMGT for IgG.IL2R67A.H1.

SEQ ID NO:28 is the V_{H} chain for IgG.IL2R67A.H1.

SEQ ID NO:29 is the heavy chain for IgG.IL2R67A.H1.

SEQ ID NO:30 is the LCDR1 kabat for IgG.IL2R67A.H1.

SEQ ID NO:31 is the LCDR2 kabat for IgG.IL2R67A.H1.

SEQ ID NO:32 is the LCDR3 kabat for IgG.IL2R67A.H1.

SEQ ID NO:33 is the LCDR1 chothia for IgG.IL2R67A.H1.

SEQ ID NO:34 is the LCDR2 chothia for IgG.IL2R67A.H1.

SEQ ID NO:35 is the LCDR3 chothia for IgG.IL2R67A.H1.

SEQ ID NO: 36 is a V_{L} chain.

SEQ ID NO:37 is a light chain.

SEQ ID NO:38 is a light chain.

SEQ ID NO: 39 is a light chain.

SEQ ID NO:40 is the amino acid sequence of human 4-1BB.

SEQ ID NO:41 is the amino acid sequence of murine 4-1BB.

SEQ ID NO: 42 is the heavy chain for the 4-1BB agonist monoclonal antibody utomilumab (PF-05082566).

SEQ ID NO:43 is the light chain for the 4-1BB agonist monoclonal antibody utomilumab (PF-05082566).

SEQ ID NO:44 is the heavy chain variable region (V_{H}) for the 4-1BB agonist monoclonal antibody utomilumab (PF-05082566).

SEQ ID NO:45 is the light chain variable region (V_{L}) for the 4-1BB agonist monoclonal antibody utomilumab (PF-05082566).

SEQ ID NO:46 is the heavy chain CDR1 for the 4-1BB agonist monoclonal antibody utomilumab (PF-05082566).

SEQ ID NO:47 is the heavy chain CDR2 for the 4-1BB agonist monoclonal antibody utomilumab (PF-05082566).

SEQ ID NO:48 is the heavy chain CDR3 for the 4-1BB agonist monoclonal antibody utomilumab (PF-05082566).

SEQ ID NO:49 is the light chain CDR1 for the 4-1BB agonist monoclonal antibody utomilumab (PF-05082566).

SEQ ID NO:50 is the light chain CDR2 for the 4-1BB agonist monoclonal antibody utomilumab (PF-05082566).

SEQ ID NO:51 is the light chain CDR3 for the 4-1BB agonist monoclonal antibody utomilumab (PF-05082566).

SEQ ID NO:52 is the heavy chain for the 4-1BB agonist monoclonal antibody urelumab (BMS-663513).

SEQ ID NO:53 is the light chain for the 4-1BB agonist monoclonal antibody urelumab (BMS-663513).

SEQ ID NO:54 is the heavy chain variable region (VH) for the 4-1BB agonist monoclonal antibody urelumab (BMS-663513).

SEQ ID NO:55 is the light chain variable region (VL) for the 4-1BB agonist monoclonal antibody urelumab (BMS-663513).

SEQ ID NO:56 is the heavy chain CDR1 for the 4-1BB agonist monoclonal antibody urelumab (BMS-663513).

SEQ ID NO:57 is the heavy chain CDR2 for the 4-1BB agonist monoclonal antibody urelumab (BMS-663513).

SEQ ID NO:58 is the heavy chain CDR3 for the 4-1BB agonist monoclonal antibody urelumab (BMS-663513).

SEQ ID NO:59 is the light chain CDR1 for the 4-1BB agonist monoclonal antibody urelumab (BMS-663513).

SEQ ID NO:60 is the light chain CDR2 for the 4-1BB agonist monoclonal antibody urelumab (BMS-663513).

SEQ ID NO:61 is the light chain CDR3 for the 4-1BB agonist monoclonal antibody urelumab (BMS-663513).

SEQ ID NO:62 is an Fc domain for a TNFRSF agonist fusion protein.

SEQ ID NO: 63 is a linker for a TNFRSF agonist fusion protein.

SEQ ID NO: 64 is a linker for a TNFRSF agonist fusion protein.

SEQ ID NO: 65 is a linker for a TNFRSF agonist fusion protein.

SEQ ID NO: 66 is a linker for a TNFRSF agonist fusion protein.

SEQ ID NO: 67 is a linker for a TNFRSF agonist fusion protein.

SEQ ID NO: 68 is a linker for a TNFRSF agonist fusion protein.

SEQ ID NO: 69 is a linker for a TNFRSF agonist fusion protein.

SEQ ID NO: 70 is a linker for a TNFRSF agonist fusion protein.

SEQ ID NO:71 is a linker for a TNFRSF agonist fusion protein.

SEQ ID NO: 72 is a linker for a TNFRSF agonist fusion protein.

SEQ ID NO: 73 is an Fc domain for a TNFRSF agonist fusion protein.

SEQ ID NO: 74 is a linker for a TNFRSF agonist fusion protein.

SEQ ID NO: 75 is a linker for a TNFRSF agonist fusion protein.

SEQ ID NO: 76 is a linker for a TNFRSF agonist fusion protein.

SEQ ID NO:77 is a 4-1BB ligand (4-1BBL) amino acid sequence.

SEQ ID NO:78 is a soluble portion of 4-1BBL polypeptide.

SEQ ID NO:79 is a heavy chain variable region (V_{H}) for the 4-1BB agonist antibody 4B4-1-1 version 1.

SEQ ID NO:80 is a light chain variable region (V_{L}) for the 4-1BB agonist antibody 4B4-1-1 version 1.

SEQ ID NO:81 is a heavy chain variable region (V_{H}) for the 4-1BB agonist antibody 4B4-1-1 version 2.

SEQ ID NO:82 is a light chain variable region (V_{L}) for the 4-1BB agonist antibody 4B4-1-1 version 2.

SEQ ID NO:83 is a heavy chain variable region (V_{H}) for the 4-1BB agonist antibody H39E3-2.

SEQ ID NO:84 is a light chain variable region (V_{L}) for the 4-1BB agonist antibody H39E3-2.

SEQ ID NO:85 is the amino acid sequence of human OX40.

SEQ ID NO: 86 is the amino acid sequence of murine OX40.

SEQ ID NO:87 is the heavy chain for the OX40 agonist monoclonal antibody tavolixizumab (MEDI-0562).

SEQ ID NO:88 is the light chain for the OX40 agonist monoclonal antibody tavolixizumab (MEDI-0562).

SEQ ID NO:89 is the heavy chain variable region (V_{H}) for the OX40 agonist monoclonal antibody tavolixizumab (MEDI-0562).

SEQ ID NO:90 is the light chain variable region (V_{L}) for the OX40 agonist monoclonal antibody tavolixizumab (MEDI-0562).

SEQ ID NO:91 is the heavy chain CDR1 for the OX40 agonist monoclonal antibody tavolixizumab (MEDI-0562).

SEQ ID NO:92 is the heavy chain CDR2 for the OX40 agonist monoclonal antibody tavolixizumab (MEDI-0562).

SEQ ID NO:93 is the heavy chain CDR3 for the OX40 agonist monoclonal antibody tavolixizumab (MEDI-0562).

SEQ ID NO:94 is the light chain CDR1 for the OX40 agonist monoclonal antibody tavolixizumab (MEDI-0562).

SEQ ID NO:95 is the light chain CDR2 for the OX40 agonist monoclonal antibody tavolixizumab (MEDI-0562).

SEQ ID NO:96 is the light chain CDR3 for the OX40 agonist monoclonal antibody tavolixizumab (MEDI-0562).

SEQ ID NO:97 is the heavy chain for the OX40 agonist monoclonal antibody 11D4.

SEQ ID NO:98 is the light chain for the OX40 agonist monoclonal antibody 11D4.

SEQ ID NO: 99 is the heavy chain variable region (V_{H}) for the OX40 agonist monoclonal antibody 11D4.

SEQ ID NO:100 is the light chain variable region (V_{L}) for the OX40 agonist monoclonal antibody 11D4.

SEQ ID NO:101 is the heavy chain CDR1 for the OX40 agonist monoclonal antibody 11D4.

SEQ ID NO: 102 is the heavy chain CDR2 for the OX40 agonist monoclonal antibody 11D4.

SEQ ID NO: 103 is the heavy chain CDR3 for the OX40 agonist monoclonal antibody 11D4.

SEQ ID NO:104 is the light chain CDR1 for the OX40 agonist monoclonal antibody 11D4.

SEQ ID NO:105 is the light chain CDR2 for the OX40 agonist monoclonal antibody 11D4.

SEQ ID NO: 106 is the light chain CDR3 for the OX40 agonist monoclonal antibody 11D4.

SEQ ID NO: 107 is the heavy chain for the OX40 agonist monoclonal antibody 18D8.

SEQ ID NO: 108 is the light chain for the OX40 agonist monoclonal antibody 18D8.

SEQ ID NO: 109 is the heavy chain variable region (V_{H}) for the OX40 agonist monoclonal antibody 18D8.

SEQ ID NO:110 is the light chain variable region (V_{L}) for the OX40 agonist monoclonal antibody 18D8.

SEQ ID NO: 111 is the heavy chain CDR1 for the OX40 agonist monoclonal antibody 18D8.

SEQ ID NO: 112 is the heavy chain CDR2 for the OX40 agonist monoclonal antibody 18D8.

SEQ ID NO:113 is the heavy chain CDR3 for the OX40 agonist monoclonal antibody 18D8.

SEQ ID NO:114 is the light chain CDR1 for the OX40 agonist monoclonal antibody 18D8.

SEQ ID NO:115 is the light chain CDR2 for the OX40 agonist monoclonal antibody 18D8.

SEQ ID NO: 116 is the light chain CDR3 for the OX40 agonist monoclonal antibody 18D8.

SEQ ID NO: 117 is the heavy chain variable region (V_{H}) for the OX40 agonist monoclonal antibody Hu119-122.

SEQ ID NO:118 is the light chain variable region (V_{L}) for the OX40 agonist monoclonal antibody Hu119-122.

SEQ ID NO:119 is the heavy chain CDR1 for the OX40 agonist monoclonal antibody Hu119-122.

SEQ ID NO:120 is the heavy chain CDR2 for the OX40 agonist monoclonal antibody Hu119-122.

SEQ ID NO:121 is the heavy chain CDR3 for the OX40 agonist monoclonal antibody Hu119-122.

SEQ ID NO:122 is the light chain CDR1 for the OX40 agonist monoclonal antibody Hu119-122.

SEQ ID NO:123 is the light chain CDR2 for the OX40 agonist monoclonal antibody Hu119-122.

SEQ ID NO: 124 is the light chain CDR3 for the OX40 agonist monoclonal antibody Hu119-122.

SEQ ID NO: 125 is the heavy chain variable region (V_{H}) for the OX40 agonist monoclonal antibody Hu106-222.

SEQ ID NO: 126 is the light chain variable region (V_{L}) for the OX40 agonist monoclonal antibody Hu106-222.

SEQ ID NO:127 is the heavy chain CDR1 for the OX40 agonist monoclonal antibody Hu106-222.

SEQ ID NO:128 is the heavy chain CDR2 for the OX40 agonist monoclonal antibody Hu106-222.

SEQ ID NO:129 is the heavy chain CDR3 for the OX40 agonist monoclonal antibody Hu106-222.

SEQ ID NO:130 is the light chain CDR1 for the OX40 agonist monoclonal antibody Hu106-222.

SEQ ID NO:131 is the light chain CDR2 for the OX40 agonist monoclonal antibody Hu106-222.

SEQ ID NO:132 is the light chain CDR3 for the OX40 agonist monoclonal antibody Hu106-222.

SEQ ID NO:133 is an OX40 ligand (OX40L) amino acid sequence.

SEQ ID NO: 134 is a soluble portion of OX40L polypeptide.

SEQ ID NO: 135 is an alternative soluble portion of OX40L polypeptide.

SEQ ID NO: 136 is the heavy chain variable region (V_{H}) for the OX40 agonist monoclonal antibody 008.

SEQ ID NO:137 is the light chain variable region (V_{L}) for the OX40 agonist monoclonal antibody 008.

SEQ ID NO:138 is the heavy chain variable region (V_{H}) for the OX40 agonist monoclonal antibody 011.

SEQ ID NO:139 is the light chain variable region (V_{L}) for the OX40 agonist monoclonal antibody 011.

SEQ ID NO:140 is the heavy chain variable region (V_{H}) for the OX40 agonist monoclonal antibody 021.

SEQ ID NO:141 is the light chain variable region (V_{L}) for the OX40 agonist monoclonal antibody 021.

SEQ ID NO:142 is the heavy chain variable region (V_{H}) for the OX40 agonist monoclonal antibody 023.

SEQ ID NO:143 is the light chain variable region (V_{L}) for the OX40 agonist monoclonal antibody 023.

SEQ ID NO:144 is the heavy chain variable region (V_{H}) for an OX40 agonist monoclonal antibody.

SEQ ID NO: 145 is the light chain variable region (V_{L}) for an OX40 agonist monoclonal antibody.

SEQ ID NO: 146 is the heavy chain variable region (V_{H}) for an OX40 agonist monoclonal antibody.

SEQ ID NO: 147 is the light chain variable region (V_{L}) for an OX40 agonist monoclonal antibody.

SEQ ID NO:148 is the heavy chain variable region (V_{H}) for a humanized OX40 agonist monoclonal antibody.

SEQ ID NO:149 is the heavy chain variable region (V_{H}) for a humanized OX40 agonist monoclonal antibody.

SEQ ID NO:150 is the light chain variable region (V_{L}) for a humanized OX40 agonist monoclonal antibody.

SEQ ID NO:151 is the light chain variable region (V_{L}) for a humanized OX40 agonist monoclonal antibody.

SEQ ID NO:152 is the heavy chain variable region (V_{H}) for a humanized OX40 agonist monoclonal antibody.

SEQ ID NO:153 is the heavy chain variable region (V_{H}) for a humanized OX40 agonist monoclonal antibody.

SEQ ID NO:154 is the light chain variable region (V_{L}) for a humanized OX40 agonist monoclonal antibody.

SEQ ID NO:155 is the light chain variable region (V_{L}) for a humanized OX40 agonist monoclonal antibody.

SEQ ID NO:156 is the heavy chain variable region (V_{H}) for an OX40 agonist monoclonal antibody.

SEQ ID NO:157 is the light chain variable region (V_{L}) for an OX40 agonist monoclonal antibody.

SEQ ID NO:158 is the heavy chain amino acid sequence of the PD-1 inhibitor nivolumab.

SEQ ID NO:159 is the light chain amino acid sequence of the PD-1 inhibitor nivolumab.

SEQ ID NO:160 is the heavy chain variable region (V_{H}) amino acid sequence of the PD-1 inhibitor nivolumab.

SEQ ID NO:161 is the light chain variable region (V_{L}) amino acid sequence of the PD-1 inhibitor nivolumab.

SEQ ID NO:162 is the heavy chain CDR1 amino acid sequence of the PD-1 inhibitor nivolumab.

SEQ ID NO:163 is the heavy chain CDR2 amino acid sequence of the PD-1 inhibitor nivolumab.

SEQ ID NO:164 is the heavy chain CDR3 amino acid sequence of the PD-1 inhibitor nivolumab.

SEQ ID NO:165 is the light chain CDR1 amino acid sequence of the PD-1 inhibitor nivolumab.

SEQ ID NO:166 is the light chain CDR2 amino acid sequence of the PD-1 inhibitor nivolumab.

SEQ ID NO:167 is the light chain CDR3 amino acid sequence of the PD-1 inhibitor nivolumab.

SEQ ID NO:168 is the heavy chain amino acid sequence of the PD-1 inhibitor pembrolizumab.

SEQ ID NO:169 is the light chain amino acid sequence of the PD-1 inhibitor pembrolizumab.

SEQ ID NO:170 is the heavy chain variable region (V_{H}) amino acid sequence of the PD-1 inhibitor pembrolizumab.

SEQ ID NO:171 is the light chain variable region (V_{L}) amino acid sequence of the PD-1 inhibitor pembrolizumab.

SEQ ID NO:172 is the heavy chain CDR1 amino acid sequence of the PD-1 inhibitor pembrolizumab.

SEQ ID NO:173 is the heavy chain CDR2 amino acid sequence of the PD-1 inhibitor pembrolizumab.

SEQ ID NO:174 is the heavy chain CDR3 amino acid sequence of the PD-1 inhibitor pembrolizumab.

SEQ ID NO:175 is the light chain CDR1 amino acid sequence of the PD-1 inhibitor pembrolizumab.

SEQ ID NO: 176 is the light chain CDR2 amino acid sequence of the PD-1 inhibitor pembrolizumab.

SEQ ID NO:177 is the light chain CDR3 amino acid sequence of the PD-1 inhibitor pembrolizumab.

SEQ ID NO:178 is the heavy chain amino acid sequence of the PD-L1 inhibitor durvalumab.

SEQ ID NO:179 is the light chain amino acid sequence of the PD-L1 inhibitor durvalumab.

SEQ ID NO:180 is the heavy chain variable region (V_{H}) amino acid sequence of the PD-L1 inhibitor durvalumab.

SEQ ID NO:181 is the light chain variable region (V_{L}) amino acid sequence of the PD-L1 inhibitor durvalumab.

SEQ ID NO:182 is the heavy chain CDR1 amino acid sequence of the PD-L1 inhibitor durvalumab.

SEQ ID NO:183 is the heavy chain CDR2 amino acid sequence of the PD-L1 inhibitor durvalumab.

SEQ ID NO:184 is the heavy chain CDR3 amino acid sequence of the PD-L1 inhibitor durvalumab.

SEQ ID NO:185 is the light chain CDR1 amino acid sequence of the PD-L1 inhibitor durvalumab.

SEQ ID NO:186 is the light chain CDR2 amino acid sequence of the PD-L1 inhibitor durvalumab.

SEQ ID NO:187 is the light chain CDR3 amino acid sequence of the PD-L1 inhibitor durvalumab.

SEQ ID NO:188 is the heavy chain amino acid sequence of the PD-L1 inhibitor avelumab.

SEQ ID NO:189 is the light chain amino acid sequence of the PD-L1 inhibitor avelumab.

SEQ ID NO: 190 is the heavy chain variable region (V_{H}) amino acid sequence of the PD-L1 inhibitor avelumab.

SEQ ID NO:191 is the light chain variable region (V_{L}) amino acid sequence of the PD-L1 inhibitor avelumab.

SEQ ID NO:192 is the heavy chain CDR1 amino acid sequence of the PD-L1 inhibitor avelumab.

SEQ ID NO:193 is the heavy chain CDR2 amino acid sequence of the PD-L1 inhibitor avelumab.

SEQ ID NO:194 is the heavy chain CDR3 amino acid sequence of the PD-L1 inhibitor avelumab.

SEQ ID NO:195 is the light chain CDR1 amino acid sequence of the PD-L1 inhibitor avelumab.

SEQ ID NO:196 is the light chain CDR2 amino acid sequence of the PD-L1 inhibitor avelumab.

SEQ ID NO:197 is the light chain CDR3 amino acid sequence of the PD-L1 inhibitor avelumab.

SEQ ID NO:198 is the heavy chain amino acid sequence of the PD-L1 inhibitor atezolizumab.

SEQ ID NO:199 is the light chain amino acid sequence of the PD-L1 inhibitor atezolizumab.

SEQ ID NO:200 is the heavy chain variable region (V_{H}) amino acid sequence of the PD-L1 inhibitor atezolizumab.

SEQ ID NO:201 is the light chain variable region (V_{L}) amino acid sequence of the PD-L1 inhibitor atezolizumab.

SEQ ID NO:202 is the heavy chain CDR1 amino acid sequence of the PD-L1 inhibitor atezolizumab.

SEQ ID NO:203 is the heavy chain CDR2 amino acid sequence of the PD-L1 inhibitor atezolizumab.

SEQ ID NO:204 is the heavy chain CDR3 amino acid sequence of the PD-L1 inhibitor atezolizumab.

SEQ ID NO:205 is the light chain CDR1 amino acid sequence of the PD-L1 inhibitor atezolizumab.

SEQ ID NO:206 is the light chain CDR2 amino acid sequence of the PD-L1 inhibitor atezolizumab.

SEQ ID NO:207 is the light chain CDR3 amino acid sequence of the PD-L1 inhibitor atezolizumab.

SEQ ID NO:208 is the heavy chain amino acid sequence of the CTLA-4 inhibitor ipilimumab.

SEQ ID NO:209 is the light chain amino acid sequence of the CTLA-4 inhibitor ipilimumab.

SEQ ID NO:210 is the heavy chain variable region (V_{H}) amino acid sequence of the CTLA-4 inhibitor ipilimumab.

SEQ ID NO:211 is the light chain variable region (V_{L}) amino acid sequence of the CTLA-4 inhibitor ipilimumab.

SEQ ID NO:212 is the heavy chain CDR1 amino acid sequence of the CTLA-4 inhibitor ipilimumab.

SEQ ID NO:213 is the heavy chain CDR2 amino acid sequence of the CTLA-4 inhibitor ipilimumab.

SEQ ID NO:214 is the heavy chain CDR3 amino acid sequence of the CTLA-4 inhibitor ipilimumab.

SEQ ID NO:215 is the light chain CDR1 amino acid sequence of the CTLA-4 inhibitor ipilimumab.

SEQ ID NO:216 is the light chain CDR2 amino acid sequence of the CTLA-4 inhibitor ipilimumab.

SEQ ID NO:217 is the light chain CDR3 amino acid sequence of the CTLA-4 inhibitor ipilimumab.

SEQ ID NO:218 is the heavy chain amino acid sequence of the CTLA-4 inhibitor tremelimumab.

SEQ ID NO:219 is the light chain amino acid sequence of the CTLA-4 inhibitor tremelimumab.

SEQ ID NO:220 is the heavy chain variable region (V_{H}) amino acid sequence of the CTLA-4 inhibitor tremelimumab.

SEQ ID NO:221 is the light chain variable region (V_{L}) amino acid sequence of the CTLA-4 inhibitor tremelimumab.

SEQ ID NO:222 is the heavy chain CDR1 amino acid sequence of the CTLA-4 inhibitor tremelimumab.

SEQ ID NO:223 is the heavy chain CDR2 amino acid sequence of the CTLA-4 inhibitor tremelimumab.

SEQ ID NO:224 is the heavy chain CDR3 amino acid sequence of the CTLA-4 inhibitor tremelimumab.

SEQ ID NO:225 is the light chain CDR1 amino acid sequence of the CTLA-4 inhibitor tremelimumab.

SEQ ID NO:226 is the light chain CDR2 amino acid sequence of the CTLA-4 inhibitor tremelimumab.

SEQ ID NO:227 is the light chain CDR3 amino acid sequence of the CTLA-4 inhibitor tremelimumab.

SEQ ID NO:228 is the heavy chain amino acid sequence of the CTLA-4 inhibitor zalifrelimab.

SEQ ID NO:229 is the light chain amino acid sequence of the CTLA-4 inhibitor zalifrelimab.

SEQ ID NO:230 is the heavy chain variable region (V_{H}) amino acid sequence of the CTLA-4 inhibitor zalifrelimab.

SEQ ID NO:231 is the light chain variable region (V_{L}) amino acid sequence of the CTLA-4 inhibitor zalifrelimab.

SEQ ID NO:232 is the heavy chain CDR1 amino acid sequence of the CTLA-4 inhibitor zalifrelimab.

SEQ ID NO:233 is the heavy chain CDR2 amino acid sequence of the CTLA-4 inhibitor zalifrelimab.

SEQ ID NO:234 is the heavy chain CDR3 amino acid sequence of the CTLA-4 inhibitor zalifrelimab.

SEQ ID NO:235 is the light chain CDR1 amino acid sequence of the CTLA-4 inhibitor zalifrelimab.

SEQ ID NO:236 is the light chain CDR2 amino acid sequence of the CTLA-4 inhibitor zalifrelimab.

SEQ ID NO:237 is the light chain CDR3 amino acid sequence of the CTLA-4 inhibitor zalifrelimab.

SEQ ID NO:238 is a CD8a transmembrane domain.

SEQ ID NO:239 is a B7-1 transmembrane-intracellular domain

SEQ ID NOs:240-245 are exemplary glycine-serine linkers that are useful in the immunomodulatory fusion proteins described herein.

SEQ ID NO:246 is an exemplary linker that is useful in the immunomodulatory fusion proteins described herein.

SEQ ID NO:247 is a 2A peptide C-terminus sequence.

SEQ ID NO:248 is a porcine teschovirus-1 2A peptide.

SEQ ID NO:249 is an equine rhinitis A virus 2A peptide.

SEQ ID NO:250 is a foot-and-mouth disease virus 2A peptide.

SEQ ID NO:251 is an exemplary furin-cleavable 2A peptide.

SEQ ID NOs:252 and 253 are human IgE signal peptide sequences. SEQ ID NO:254 is a human IL-2 signal peptide sequence.

SEQ ID NO:255 is a 6×NFAT IL-2 minimal promoter.

SEQ ID NO:256 is an NFAT responsive element.

SEQ ID NO:257 is a human IL-2 promoter sequence.

SEQ ID NO:258 is human IL-15 (N72D mutant).

SEQ ID NO:259 is human IL-15R-alpha-Su/Fc domain.

SEQ ID NO:260 is human IL-15R-alpha-Su (65aa truncated extracellular domain).

SEQ ID NO:261 is human IL-15 isoform 2.

SEQ ID NO:262 is human IL-15 isoform 1.

SEQ ID NO:263 is human IL-15 (without signal peptide).

SEQ ID NO:264 is human IL-15R-alpha (85 aa truncated extracellular domain).

SEQ ID NO:265 is human IL-15R-alpha (182aa truncated extracellular domain).

SEQ ID NO:266 is human IL-15R-alpha.

SEQ ID NO:267 is human IL-12 p35 subunit.

SEQ ID NO:268 is human IL-12 p40 subunit.

SEQ ID NO:269 is human IL-18

SEQ ID NO:270 is a human IL-18 variant

SEQ ID NO:271 is human IL-21.

SEQ ID NO: 272 is human IL-2

SEQ ID NO:273 is human CD40L

SEQ ID NO:274 is agonistic anti-human CD40 VH (Sotigalimab)

SEQ ID NO:275 is agonistic anti-human CD40 VL (Sotigalimab)

SEQ ID NO:276 is agonistic anti-human CD40 scFv (Sotigalimab)

SEQ ID NO:277 is agonistic anti-human CD40 VH (Dacetuzumab)

SEQ ID NO:278 is agonistic anti-human CD40 VL (Dacetuzumab)

SEQ ID NO:279 is agonistic anti-human CD40 scFv (Dacetuzumab)

SEQ ID NO:280 is agonistic anti-human CD40 VH (Lucatutuzumab)

SEQ ID NO:281 is agonistic anti-human CD40 VL (Lucatutuzumab)

SEQ ID NO:282 is agonistic anti-human CD40 scFv (Lucatutuzumab)

SEQ ID NO:283 is agonistic anti-human CD40 VH (Selicrelumab)

SEQ ID NO:284 is agonistic anti-human CD40 VL (Selicrelumab)

SEQ ID NO:285 is agonistic anti-human CD40 scFv (Selicrelumab)

SEQ ID NO:286 is a target PD-1 sequence.

SEQ ID NO:287 is a target PD-1 sequence.

SEQ ID NO:288 is a repeat PD-1 left repeat sequence.

SEQ ID NO:289 is a repeat PD-1 right repeat sequence.

SEQ ID NO: 290 is a repeat PD-1 left repeat sequence.

SEQ ID NO: 291 is a repeat PD-1 right repeat sequence.

SEQ ID NO:292 is a PD-1 left TALEN nuclease sequence.

SEQ ID NO: 293 is a PD-1 right TALEN nuclease sequence.

SEQ ID NO:294 is a PD-1 left TALEN nuclease sequence.

SEQ ID NO: 295 is a PD-1 right TALEN nuclease sequence.

SEQ ID NO: 296 is a nucleic acid sequence that encodes for the tethered IL-15 of SEQ ID NO:328

SEQ ID NO:297 is a nucleic acid sequence that encodes for the tethered IL-21 fusion protein of SEQ ID NO.

SEQ ID NO:298 is a nucleic acid sequence that encodes for the tethered IL-15 fusion protein of SEQ ID NO:328 and tether IL-21 fusion protein of SEQ ID NO:331.

SEQ ID NO:299 is a nucleic acid sequence that encodes for the tethered IL-12 fusion protein of SEQ ID NO:303. The nucleic acid sequence includes an NFAT promoter.

SEQ ID NO:300 is a nucleic acid sequence that encodes for the tethered IL-15 fusion protein of SEQ ID NO:328. The nucleic acid sequence includes an NFAT promoter.

SEQ ID NO:301 is a nucleic acid sequence that encodes for the tethered IL-21 fusion protein of SEQ ID NO:XX. The nucleic acid sequence includes an NFAT promoter.

SEQ ID NO:302 is a nucleic acid sequence that encodes for the tethered IL-15 fusion protein of SEQ ID NO:328 and tether IL-21 fusion protein of SEQ ID NO:331. The nucleic acid sequence includes an NFAT promoter.

SEQ ID NO:303 is the amino acid sequence of an exemplary tethered IL-12 (tethered IL-12-Lr1-Ar2).

SEQ ID NO:304 is a nucleic acid sequence that encodes for the tethered IL-12 of SEQ ID NO:303.

SEQ ID NO:305 is the amino acid sequence of an exemplary tethered IL-18 (tethered IL-18-Lr1-Ar2).

SEQ ID NO:306 is a nucleic acid sequence that encodes for the tethered IL-18 of SEQ ID NO:305.

SEQ ID NO:307 is the amino acid sequence of an exemplary tethered variant IL-18 (tethered DR-IL-18 (6-27 variant)-Lr1-Ar2).

SEQ ID NO:308 is a nucleic acid sequence that encodes for the tethered variant IL-18 of SEQ ID NO:307.

SEQ ID NO:309 is the amino acid sequence of an exemplary tethered IL-12/IL-15.

SEQ ID NO:310 is a nucleic acid sequence that encodes for the tethered IL-12/IL-15 of SEQ ID NO:309.

SEQ ID NO:311 is the amino acid sequence of an exemplary tethered IL-18/IL-15.

SEQ ID NO:312 is a nucleic acid sequence that encodes for the tethered IL-18/IL-15 of SEQ ID NO:311.

SEQ ID NO:313 is the amino acid sequence of an exemplary tethered anti-CD40scFV (APX005M).

SEQ ID NO:314 is a nucleic acid sequence that encodes for the tethered anti-CD40scFV (APX005M) of SEQ ID NO:313.

SEQ ID NO:315 is the amino acid sequence of an exemplary tethered anti-CD40scFV (Dacetuzumab).

SEQ ID NO:316 is a nucleic acid sequence that encodes for the tethered anti-CD40scFV (Dacetuzumab) of SEQ ID NO:315.

SEQ ID NO:317 is the amino acid sequence of an exemplary tethered anti-CD40scFV (Lucatutuzumab).

SEQ ID NO:318 is a nucleic acid sequence that encodes for the tethered anti-CD40scFV (Lucatutuzumab) of SEQ ID NO:317.

SEQ ID NO:319 is the amino acid sequence of an exemplary tethered anti-CD40scFV (Selicrelumab).

SEQ ID NO:320 is a nucleic acid sequence that encodes for the tethered anti-CD40scFV (Selicrelumab) of SEQ ID NO:319.

SEQ ID NO:321 is a nucleic acid sequence that encodes for the CD40L of SEQ ID NO:273.

SEQ ID NO:322 is the amino acid sequence an exemplary tethered CD40L/IL-15.

SEQ ID NO:323 is a nucleic acid sequence that encodes for the tethered CD40L/IL-15 of SEQ ID NO:311.

SEQ ID NO:324 is the amino acid sequence of an exemplary tethered IL-2.

SEQ ID NO:325 is a nucleic acid sequence that encodes for the tethered IL-2 of SEQ ID NO:313.

SEQ ID NO:326 is the amino acid sequence of an exemplary tethered IL-12.

SEQ ID NO:327 is a nucleic acid sequence that encodes for the tethered IL-12 of SEQ ID NO:315.

SEQ ID NO:328 is the amino acid sequence of an exemplary tethered IL-15.

SEQ ID NO:329 is a nucleic acid sequence that encodes for the tethered IL-15 of SEQ ID NO:317.

SEQ ID NO:330 is a nucleic acid sequence that encodes for GFP.

SEQ ID NOS:331-385 are nucleic acids of additional variant IL-18s (e.g., decoy-resistant IL-18s or “DR-IL18”).

SEQ ID NO:386 is an exemplary Clo05 1 nuclease domain amino acid sequence.

SEQ ID NO:387 is an exemplary piggyBac (PB) transposase enzyme amino acid sequence.

SEQ ID NO:388 is an exemplary Sleeping Beauty transposase enzyme amino acid sequence.

SEQ ID NO:389 is an exemplary hyperactive Sleeping Beauty (SB100X) transposase amino acid sequence.

SEQ ID NO:390 is an exemplary nucleic acid sequence of a 6×NFAT binding motif.

SEQ ID NO:391 is an exemplary nucleic acid sequence of an IL-2 min promoter.

SEQ ID NO:392 is an Tethered IL-12 (TeIL-12).

SEQ ID NO:393 is an exemplary nucleic acid sequence of an IRES.

SEQ ID NO:394 is an exemplary nucleic acid sequence of a U6 promoter.

SEQ ID NO:395-SEQ ID NO:401 are exemplary nucleic acid sequence of PD-1 shRNAs.

### DETAILED DESCRIPTION

### I. Introduction

Adoptive cell therapy utilizing TILs is an effective approach for inducing tumor regression in various cancers, including leukemias and melanoma. The use of adjuvants that include immunostimulatory agents has been explored to enhance adoptive cell therapies and to extend such therapies to other solid tumors. Co-administration of immunomodulators such as cytokines (e.g., interleukins), however, can lead to undesirably toxicity due to the high dosages required. Thus, supplying such adjuvants at the right time and site appears crucial to avoid such undesirable effects.

Provided herein are compositions and methods for the treatment of cancers using modified TILs, wherein the modified TILs include one or more immunomodulatory agents (e.g., cytokines) associated with their cell surface. The immunomodulatory agents associated with the TILs provide a localized immunostimulatory effect that can advantageously enhance TIL survival and/or anti-tumor activity in a patient recipient. As such, the compositions and methods disclosed herein provide effective cancer therapies.

### II. Definitions

Unless defined otherwise, all technical and scientific terms used herein have the same meaning as is commonly understood by one of skill in the art to which this invention belongs. All patents and publications referred to herein are incorporated by reference in their entireties.

The terms “co-administration,” “co-administering,” “administered in combination with,” “administering in combination with,” “simultaneous,” and “concurrent,” as used herein, encompass administration of two or more active pharmaceutical ingredients (in a preferred embodiment of the present invention, for example, a plurality of TILs) to a subject so that both active pharmaceutical ingredients and/or their metabolites are present in the subject at the same time. Co-administration includes simultaneous administration in separate compositions, administration at different times in separate compositions, or administration in a composition in which two or more active pharmaceutical ingredients are present. Simultaneous administration in separate compositions and administration in a composition in which both agents are present are preferred.

The term “in vivo” refers to an event that takes place in a subject's body.

The term “in vitro” refers to an event that takes places outside of a subject's body. In vitro assays encompass cell-based assays in which cells alive or dead are employed and may also encompass a cell-free assay in which no intact cells are employed.

The term “ex vivo” refers to an event which involves treating or performing a procedure on a cell, tissue and/or organ which has been removed from a subject's body. Aptly, the cell, tissue and/or organ may be returned to the subject's body in a method of surgery or treatment.

The term “rapid expansion” means an increase in the number of antigen-specific TILs of at least about 3-fold (or 4-, 5-, 6-, 7-, 8-, or 9-fold) over a period of a week, more preferably at least about 10-fold (or 20-, 30-, 40-, 50-, 60-, 70-, 80-, or 90-fold) over a period of a week, or most preferably at least about 100-fold over a period of a week. A number of rapid expansion protocols are described herein.

By “tumor infiltrating lymphocytes” or “TILs” herein is meant a population of cells originally obtained as white blood cells that have left the bloodstream of a subject and migrated into a tumor. TILs include, but are not limited to, CD8+ cytotoxic T cells (lymphocytes), Th1 and Th17 CD4+ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs and expanded TILs (“REP TILs” or “post-REP TILs”). TIL cell populations can include genetically modified TILs.

By “population of cells” (including TILs) herein is meant a number of cells that share common traits. In general, populations generally range from 1×10⁶ to 1×10¹⁰ in number, with different TIL populations comprising different numbers. For example, initial growth of primary TILs in the presence of IL-2 results in a population of bulk TILs of roughly 1×10⁸ cells. REP expansion is generally done to provide populations of 1.5×10⁹ to 1.5×10¹⁰ cells for infusion.

By “cryopreserved TILs” herein is meant that TILs, either primary, bulk, or expanded (REP TILs), are treated and stored in the range of about −150° C. to −60° C. General methods for cryopreservation are also described elsewhere herein, including in the Examples. For clarity, “cryopreserved TILs” are distinguishable from frozen tissue samples which may be used as a source of primary TILs.

By “thawed cryopreserved TILs” herein is meant a population of TILs that was previously cryopreserved and then treated to return to room temperature or higher, including but not limited to cell culture temperatures or temperatures wherein TILs may be administered to a patient.

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally and alternatively, TILs can be functionally defined by their ability to infiltrate solid tumors upon reintroduction into a patient.

The term “cryopreservation media” or “cryopreservation medium” refers to any medium that can be used for cryopreservation of cells. Such media can include media comprising 7% to 10% DMSO. Exemplary media include CryoStor CS10, Hyperthermasol, as well as combinations thereof. The term “CS10” refers to a cryopreservation medium which is obtained from Stemcell Technologies or from Biolife Solutions. The CS10 medium may be referred to by the trade name “CryoStor® CS10”. The CS10 medium is a serum-free, animal component-free medium which comprises DMSO.

The term “central memory T cell” refers to a subset of T cells that in the human are CD45R0+ and constitutively express CCR7 (CCR7^{hi}) and CD62L (CD62^{hi}). The surface phenotype of central memory T cells also includes TCR, CD3, CD127 (IL-7R), and IL-15R. Transcription factors for central memory T cells include BCL-6, BCL-6B, MBD2, and BMI1. Central memory T cells primarily secret IL-2 and CD40L as effector molecules after TCR triggering. Central memory T cells are predominant in the CD4 compartment in blood, and in the human are proportionally enriched in lymph nodes and tonsils.

The term “effector memory T cell” refers to a subset of human or mammalian T cells that, like central memory T cells, are CD45R0+, but have lost the constitutive expression of CCR7 (CCR71°) and are heterogeneous or low for CD62L expression (CD62L^{lo}). The surface phenotype of central memory T cells also includes TCR, CD3, CD127 (IL-7R), and IL-15R. Transcription factors for central memory T cells include BLIMP1. Effector memory T cells rapidly secret high levels of inflammatory cytokines following antigenic stimulation, including interferon-γ, IL-4, and IL-5. Effector memory T cells are predominant in the CD8 compartment in blood, and in the human are proportionally enriched in the lung, liver, and gut. CD8+ effector memory T cells carry large amounts of perform.

The term “closed system” refers to a system that is closed to the outside environment. Any closed system appropriate for cell culture methods can be employed with the methods of the present invention. Closed systems include, for example, but are not limited to, closed G-containers. Once a tumor segment is added to the closed system, the system is no opened to the outside environment until the TILs are ready to be administered to the patient.

The terms “fragmenting,” “fragment,” and “fragmented,” as used herein to describe processes for disrupting a tumor, includes mechanical fragmentation methods such as crushing, slicing, dividing, and morcellating tumor tissue as well as any other method for disrupting the physical structure of tumor tissue.

The terms “peripheral blood mononuclear cells” and “PBMCs” refers to a peripheral blood cell having a round nucleus, including lymphocytes (T cells, B cells, NK cells) and monocytes. When used as an antigen presenting cell (PBMCs are a type of antigen-presenting cell), the peripheral blood mononuclear cells are preferably irradiated allogeneic peripheral blood mononuclear cells.

The terms “peripheral blood lymphocytes” and “PBLs” refer to T cells expanded from peripheral blood. In some embodiments, PBLs are separated from whole blood or apheresis product from a donor. In some embodiments, PBLs are separated from whole blood or apheresis product from a donor by positive or negative selection of a T cell phenotype, such as the T cell phenotype of CD3+ CD45+.

The term “anti-CD3 antibody” refers to an antibody or variant thereof, e.g., a monoclonal antibody and including human, humanized, chimeric or murine antibodies which are directed against the CD3 receptor in the T cell antigen receptor of mature T cells. Anti-CD3 antibodies include OKT-3, also known as muromonab. Anti-CD3 antibodies also include the UHCT1 clone, also known as T3 and CD3ε. Other anti-CD3 antibodies include, for example, otelixizumab, teplizumab, and visilizumab.

The term “OKT-3” (also referred to herein as “OKT3”) refers to a monoclonal antibody or biosimilar or variant thereof, including human, humanized, chimeric, or murine antibodies, directed against the CD3 receptor in the T cell antigen receptor of mature T cells, and includes commercially-available forms such as OKT-3 (30 ng/mL, MACS GMP CD3 pure, Miltenyi Biotech, Inc., San Diego, CA, USA) and muromonab or variants, conservative amino acid substitutions, glycoforms, or biosimilars thereof. The amino acid sequences of the heavy and light chains of muromonab are given in Table 1 (SEQ ID NO:1 and SEQ ID NO:2). A hybridoma capable of producing OKT-3 is deposited with the American Type Culture Collection and assigned the ATCC accession number CRL 8001. A hybridoma capable of producing OKT-3 is also deposited with European Collection of Authenticated Cell Cultures (ECACC) and assigned Catalogue No. 86022706.

TABLE 1
Amino acid sequences of muromonab (exemplary OKT-3 antibody).

| Identifier | Sequence (One-Letter Amino Acid Symbols) | |
|---|---|---|
| SEQ ID NO: 1 | QVQLQQSGAE LARPGASVKM SCKASGYTFT RYTMHWVKQR PGQGLEWIGY INPSRGYTNY | 60 |
| muromonab heavy | NQKFKDKATL TTDKSSSTAY MQLSSLTSED SAVYYCARYY DDHYCLDYWG QGTTLTVSSA | 120 |
| chain | KTTAPSVYPL APVCGGTTGS SVTLGCLVKG YFPEPVTLTW NSGSLSSGVH TFPAVLQSDL | 180 |
| | YTLSSSVTVT SSTWPSQSIT CNVAHPASST KVDKKIEPRP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWLNGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 2 | QIVLTQSPAI MSASPGEKVT MTCSASSSVS YMNWYQQKSG TSPKRWIYDT SKLASGVPAH | 60 |
| muromonab light | FRGSGSGTSY SLTISGMEAE DAATYYCQQW SSNPFTFGSG TKLEINRADT APTVSIFPPS | 120 |
| chain | SEQLTSGGAS VVCFLNNFYP KDINVKWKID GSERQNGVLN SWTDQDSKDS TYSMSSTLTL | 180 |
| | TKDEYERHNS YTCEATHKTS TSPIVKSFNR NEC | 213 |

The term “IL-2” (also referred to herein as “IL2”) refers to the T cell growth factor known as interleukin-2, and includes all forms of IL-2 including human and mammalian forms, conservative amino acid substitutions, glycoforms, biosimilars, and variants thereof. IL-2 is described, e.g., in Nelson, J. Immunol. 2004, 172, 3983-88 and Malek, Annu. Rev. Immunol. 2008, 26, 453-79, the disclosures of which are incorporated by reference herein. The amino acid sequence of recombinant human IL-2 suitable for use in the invention is given in Table 2 (SEQ ID NO:3). For example, the term IL-2 encompasses human, recombinant forms of IL-2 such as aldesleukin (PROLEUKIN, available commercially from multiple suppliers in 22 million IU per single use vials), as well as the form of recombinant IL-2 commercially supplied by CellGenix, Inc., Portsmouth, NH, USA (CELLGRO GMP) or ProSpec-Tany TechnoGene Ltd., East Brunswick, NJ, USA (Cat. No. CYT-209-b) and other commercial equivalents from other vendors. Aldesleukin (des-alanyl-1, serine-125 human IL-2) is a nonglycosylated human recombinant form of IL-2 with a molecular weight of approximately 15 kDa. The amino acid sequence of aldesleukin suitable for use in the invention is given in Table 2 (SEQ ID NO:4). The term IL-2 also encompasses pegylated forms of IL-2, as described herein, including the pegylated IL2 prodrug bempegaldesleukin (NKTR-214, pegylated human recombinant IL-2 as in SEQ ID NO:4 in which an average of 6 lysine residues are N⁶ substituted with [(2,7-bis{[methylpoly(oxyethylene)]carbamoyl}-9H-fluoren-9-yl)methoxy]carbonyl), which is available from Nektar Therapeutics, South San Francisco, CA, USA, or which may be prepared by methods known in the art, such as the methods described in Example 19 of International Patent Application Publication No. WO 2018/132496 A1 or the method described in Example 1 of U.S. Patent Application Publication No. US 2019/0275133 A1, the disclosures of which are incorporated by reference herein. Bempegaldesleukin (NKTR-214) and other pegylated IL-2 molecules suitable for use in the invention are described in U.S. Patent Application Publication No. US 2014/0328791 A1 and International Patent Application Publication No. WO 2012/065086 A1, the disclosures of which are incorporated by reference herein. Alternative forms of conjugated IL-2 suitable for use in the invention are described in U.S. Pat. Nos. 4,766,106, 5,206,344, 5,089,261 and 4,902,502, the disclosures of which are incorporated by reference herein. Formulations of IL-2 suitable for use in the invention are described in U.S. Pat. No. 6,706,289, the disclosure of which is incorporated by reference herein.

In some embodiments, an IL-2 form suitable for use in the present invention is THOR-707, available from Synthorx, Inc. The preparation and properties of THOR-707 and additional alternative forms of IL-2 suitable for use in the invention are described in U.S. Patent Application Publication Nos. US 2020/0181220 A1 and US 2020/0330601 A1, the disclosures of which are incorporated by reference herein. In some embodiments, and IL-2 form suitable for use in the invention is an interleukin 2 (IL-2) conjugate comprising: an isolated and purified IL-2 polypeptide; and a conjugating moiety that binds to the isolated and purified IL-2 polypeptide at an amino acid position selected from K35, T37, R38, T41, F42, K43, F44, Y45, E61, E62, E68, K64, P65, V69, L72, and Y107, wherein the numbering of the amino acid residues corresponds to SEQ ID NO:5. In some embodiments, the amino acid position is selected from T37, R38, T41, F42, F44, Y45, E61, E62, E68, K64, P65, V69, L72, and Y107. In some embodiments, the amino acid position is selected from T37, R38, T41, F42, F44, Y45, E61, E62, E68, P65, V69, L72, and Y107. In some embodiments, the amino acid position is selected from T37, T41, F42, F44, Y45, P65, V69, L72, and Y107. In some embodiments, the amino acid position is selected from R38 and K64. In some embodiments, the amino acid position is selected from E61, E62, and E68. In some embodiments, the amino acid position is at E62. In some embodiments, the amino acid residue selected from K35, T37, R38, T41, F42, K43, F44, Y45, E61, E62, E68, K64, P65, V69, L72, and Y107 is further mutated to lysine, cysteine, or histidine. In some embodiments, the amino acid residue is mutated to cysteine. In some embodiments, the amino acid residue is mutated to lysine. In some embodiments, the amino acid residue selected from K35, T37, R38, T41, F42, K43, F44, Y45, E61, E62, E68, K64, P65, V69, L72, and Y107 is further mutated to an unnatural amino acid. In some embodiments, the unnatural amino acid comprises N6-azidoethoxy-L-lysine (AzK), N6-propargylethoxy-L-lysine (PraK), BCN-L-lysine, norbornene lysine, TCO-lysine, methyltetrazine lysine, allyloxycarbonyllysine, 2-amino-8-oxononanoic acid, 2-amino-8-oxooctanoic acid, p-acetyl-L-phenylalanine, p-azidomethyl-L-phenylalanine (pAMF), p-iodo-L-phenylalanine, m-acetylphenylalanine, 2-amino-8-oxononanoic acid, p-propargyloxyphenylalanine, p-propargyl-phenylalanine, 3-methyl-phenylalanine, L-Dopa, fluorinated phenylalanine, isopropyl-L-phenylalanine, p-azido-L-phenylalanine, p-acyl-L-phenylalanine, p-benzoyl-L-phenylalanine, p-bromophenylalanine, p-amino-L-phenylalanine, isopropyl-L-phenylalanine, O-allyltyrosine, O-methyl-L-tyrosine, O-4-allyl-L-tyrosine, 4-propyl-L-tyrosine, phosphonotyrosine, tri-O-acetyl-GlcNAcp-serine, L-phosphoserine, phosphonoserine, L-3-(2-naphthyl)alanine, 2-amino-3-((2-((3-(benzyloxy)-3-oxopropyl)amino)ethyl)selanyl)propanoic acid, 2-amino-3-(phenylselanyl)propanoic, or selenocysteine. In some embodiments, the IL-2 conjugate has a decreased affinity to IL-2 receptor α (IL-2Rα) subunit relative to a wild-type IL-2 polypeptide. In some embodiments, the decreased affinity is about 10%, 20%, 30%, 40%, 50%, 60%, 70%, 80%, 90%, 95%, 99%, or greater than 99% decrease in binding affinity to IL-2Rα relative to a wild-type IL-2 polypeptide. In some embodiments, the decreased affinity is about 1-fold, 2-fold, 3-fold, 4-fold, 5-fold, 6-fold, 7-fold, 8-fold, 9-fold, 10-fold, 30-fold, 50-fold, 100-fold, 200-fold, 300-fold, 500-fold, 1000-fold, or more relative to a wild-type IL-2 polypeptide. In some embodiments, the conjugating moiety impairs or blocks the binding of IL-2 with IL-2Rα. In some embodiments, the conjugating moiety comprises a water-soluble polymer. In some embodiments, the additional conjugating moiety comprises a water-soluble polymer. In some embodiments, each of the water-soluble polymers independently comprises polyethylene glycol (PEG), poly(propylene glycol) (PPG), copolymers of ethylene glycol and propylene glycol, poly(oxyethylated polyol), poly(olefinic alcohol), poly(vinylpyrrolidone), poly(hydroxyalkylmethacrylamide), poly(hydroxyalkylmethacrylate), poly(saccharides), poly(α-hydroxy acid), poly(vinyl alcohol), polyphosphazene, polyoxazolines (POZ), poly(N-acryloylmorpholine), or a combination thereof. In some embodiments, each of the water-soluble polymers independently comprises PEG. In some embodiments, the PEG is a linear PEG or a branched PEG. In some embodiments, each of the water-soluble polymers independently comprises a polysaccharide. In some embodiments, the polysaccharide comprises dextran, polysialic acid (PSA), hyaluronic acid (HA), amylose, heparin, heparan sulfate (HS), dextrin, or hydroxyethyl-starch (HES). In some embodiments, each of the water-soluble polymers independently comprises a glycan. In some embodiments, each of the water-soluble polymers independently comprises polyamine. In some embodiments, the conjugating moiety comprises a protein. In some embodiments, the additional conjugating moiety comprises a protein. In some embodiments, each of the proteins independently comprises an albumin, a transferrin, or a transthyretin. In some embodiments, each of the proteins independently comprises an Fc portion. In some embodiments, each of the proteins independently comprises an Fc portion of IgG. In some embodiments, the conjugating moiety comprises a polypeptide. In some embodiments, the additional conjugating moiety comprises a polypeptide. In some embodiments, each of the polypeptides independently comprises a XTEN peptide, a glycine-rich homoamino acid polymer (HAP), a PAS polypeptide, an elastin-like polypeptide (ELP), a CTP peptide, or a gelatin-like protein (GLK) polymer. In some embodiments, the isolated and purified IL-2 polypeptide is modified by glutamylation. In some embodiments, the conjugating moiety is directly bound to the isolated and purified IL-2 polypeptide. In some embodiments, the conjugating moiety is indirectly bound to the isolated and purified IL-2 polypeptide through a linker. In some embodiments, the linker comprises a homobifunctional linker. In some embodiments, the homobifunctional linker comprises Lomant's reagent dithiobis (succinimidylpropionate) DSP, 3′3′-dithiobis(sulfosuccinimidyl proprionate) (DTSSP), disuccinimidyl suberate (DSS), bis(sulfosuccinimidyl)suberate (BS), disuccinimidyl tartrate (DST), disulfosuccinimidyl tartrate (sulfo DST), ethylene glycobis(succinimidylsuccinate) (EGS), disuccinimidyl glutarate (DSG), N,N′-disuccinimidyl carbonate (DSC), dimethyl adipimidate (DMA), dimethyl pimelimidate (DMP), dimethyl suberimidate (DMS), dimethyl-3,3′-dithiobispropionimidate (DTBP), 1,4-di-(3′-(2′-pyridyldithio)propionamido)butane (DPDPB), bismaleimidohexane (BMH), aryl halide-containing compound (DFDNB), such as e.g. 1,5-difluoro-2,4-dinitrobenzene or 1,3-difluoro-4,6-dinitrobenzene, 4,4′-difluoro-3,3′-dinitrophenylsulfone (DFDNPS), bis-[D-(4-azidosalicylamido)ethyl]disulfide (BASED), formaldehyde, glutaraldehyde, 1,4-butanediol diglycidyl ether, adipic acid dihydrazide, carbohydrazide, o-toluidine, 3,3′-dimethylbenzidine, benzidine, α,α′-p-diaminodiphenyl, diiodo-p-xylene sulfonic acid, N,N′-ethylene-bis(iodoacetamide), or N,N′-hexamethylene-bis(iodoacetamide). In some embodiments, the linker comprises a heterobifunctional linker. In some embodiments, the heterobifunctional linker comprises N-succinimidyl 3-(2-pyridyldithio)propionate (sPDP), long-chain N-succinimidyl 3-(2-pyridyldithio)propionate (LC-sPDP), water-soluble-long-chain N-succinimidyl 3-(2-pyridyldithio) propionate (sulfo-LC-sPDP), succinimidyloxycarbonyl-α-methyl-α-(2-pyridyldithio)toluene (sMPT), sulfosuccinimidyl-6-[α-methyl-α-(2-pyridyldithio)toluamido]hexanoate (sulfo-LC-sMPT), succinimidyl-4-(N-maleimidomethyl)cyclohexane-1-carboxylate (sMCC), sulfosuccinimidyl-4-(N-maleimidomethyl)cyclohexane-1-carboxylate (sulfo-sMCC), m-maleimidobenzoyl-N-hydroxysuccinimide ester (MBs), m-maleimidobenzoyl-N-hydroxysulfosuccinimide ester (sulfo-MBs), N-succinimidyl(4-iodoacteyl)aminobenzoate (sIAB), sulfosuccinimidyl(4-iodoacteyl)aminobenzoate (sulfo-sIAB), succinimidyl-4-(p-maleimidophenyl)butyrate (sMPB), sulfosuccinimidyl-4-(p-maleimidophenyl)butyrate (sulfo-sMPB), N-(γ-maleimidobutyryloxy)succinimide ester (GMBs), N-(γ-maleimidobutyryloxy) sulfosuccinimide ester (sulfo-GMBs), succinimidyl 6-((iodoacetyl)amino)hexanoate (sIAX), succinimidyl 6-[6-(((iodoacetyl)amino)hexanoyl)amino]hexanoate (slAXX), succinimidyl 4-(((iodoacetyl)amino)methyl)cyclohexane-1-carboxylate (sIAC), succinimidyl 6-(((((4-iodoacetyl)amino)methyl)cyclohexane-1-carbonyl)amino) hexanoate (sIACX), p-nitrophenyl iodoacetate (NPIA), carbonyl-reactive and sulfhydryl-reactive cross-linkers such as 4-(4-N-maleimidophenyl)butyric acid hydrazide (MPBH), 4-(N-maleimidomethyl)cyclohexane-1-carboxyl-hydrazide-8 (M2C2H), 3-(2-pyridyldithio)propionyl hydrazide (PDPH), N-hydroxysuccinimidyl-4-azidosalicylic acid (NHs-AsA), N-hydroxysulfosuccinimidyl-4-azidosalicylic acid (sulfo-NHs-AsA), sulfosuccinimidyl-(4-azidosalicylamido)hexanoate (sulfo-NHs-LC-AsA), sulfosuccinimidyl-2-(p-azidosalicylamido)ethyl-1,3′-dithiopropionate (sAsD), N-hydroxysuccinimidyl-4-azidobenzoate (HsAB), N-hydroxysulfosuccinimidyl-4-azidobenzoate (sulfo-HsAB), N-succinimidyl-6-(4′-azido-2′-nitrophenyl amino)hexanoate (sANPAH), sulfosuccinimidyl-6-(4′-azido-2′-nitrophenylamino)hexanoate (sulfo-sANPAH), N-5-azido-2-nitrobenzoyloxysuccinimide (ANB-NOs), sulfosuccinimidyl-2-(m-azido-o-nitrobenzamido)-ethyl-1,3′-dithiopropionate (sAND), N-succinimidyl-4(4-azidophenyl)1,3′-dithiopropionate (sADP), N-sulfosuccinimidyl(4-azidophenyl)-1,3′-dithiopropionate (sulfo-sADP), sulfosuccinimidyl 4-(ρ-azidophenyl)butyrate (sulfo-sAPB), sulfosuccinimidyl 2-(7-azido-4-methylcoumarin-3-acetamide)ethyl-1,3′-dithiopropionate (sAED), sulfosuccinimidyl 7-azido-4-methylcoumain-3-acetate (sulfo-sAMCA), p-nitrophenyl diazopyruvate (pNPDP), p-nitrophenyl-2-diazo-3,3,3-trifluoropropionate (PNP-DTP), 1-(ρ-azidosalicylamido)-4-(iodoacetamido)butane (AsIB), N-[4-(ρ-azidosalicylamido)butyl]-3′-(2′-pyridyldithio) propionamide (APDP), benzophenone-4-iodoacetamide, p-azidobenzoyl hydrazide (ABH), 4-(ρ-azidosalicylamido)butylamine (AsBA), or p-azidophenyl glyoxal (APG). In some embodiments, the linker comprises a cleavable linker, optionally comprising a dipeptide linker. In some embodiments, the dipeptide linker comprises Val-Cit, Phe-Lys, Val-Ala, or Val-Lys. In some embodiments, the linker comprises a non-cleavable linker. In some embodiments, the linker comprises a maleimide group, optionally comprising maleimidocaproyl (mc), succinimidyl-4-(N-maleimidomethyl)cyclohexane-1-carboxylate (sMCC), or sulfosuccinimidyl-4-(N-maleimidomethyl)cyclohexane-1-carboxylate (sulfo-sMCC). In some embodiments, the linker further comprises a spacer. In some embodiments, the spacer comprises p-aminobenzyl alcohol (PAB), p-aminobenzyoxycarbonyl (PABC), a derivative, or an analog thereof. In some embodiments, the conjugating moiety is capable of extending the serum half-life of the IL-2 conjugate. In some embodiments, the additional conjugating moiety is capable of extending the serum half-life of the IL-2 conjugate. In some embodiments, the IL-2 form suitable for use in the invention is a fragment of any of the IL-2 forms described herein. In some embodiments, the IL-2 form suitable for use in the invention is pegylated as disclosed in U.S. Patent Application Publication No. US 2020/0181220 A1 and U.S. Patent Application Publication No. US 2020/0330601 A1. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 80% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 polypeptide comprises an N-terminal deletion of one residue relative to SEQ ID NO:5. In some embodiments, the IL-2 form suitable for use in the invention lacks IL-2R alpha chain engagement but retains normal binding to the intermediate affinity IL-2R beta-gamma signaling complex. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 90% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 95% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5. In some embodiments, the IL-2 form suitable for use in the invention is an IL-2 conjugate comprising: an IL-2 polypeptide comprising an N6-azidoethoxy-L-lysine (AzK) covalently attached to a conjugating moiety comprising a polyethylene glycol (PEG), wherein: the IL-2 polypeptide comprises an amino acid sequence having at least 98% sequence identity to SEQ ID NO:5; and the AzK substitutes for an amino acid at position K35, F42, F44, K43, E62, P65, R38, T41, E68, Y45, V69, or L72 in reference to the amino acid positions within SEQ ID NO:5.

In some embodiments, an IL-2 form suitable for use in the invention is nemvaleukin alfa, also known as ALKS-4230 (SEQ ID NO:6), which is available from Alkermes, Inc. Nemvaleukin alfa is also known as human interleukin 2 fragment (1-59), variant (Cys¹²⁵>Ser⁵¹), fused via peptidyl linker (⁶⁰GG⁶¹) to human interleukin 2 fragment (62-132), fused via peptidyl linker (¹³³GSGGGS¹³⁸) to human interleukin 2 receptor α-chain fragment (139-303), produced in Chinese hamster ovary (CHO) cells, glycosylated; human interleukin 2 (IL-2) (75-133)-peptide [Cys¹²⁵(51)>Ser]-mutant (1-59), fused via a G2 peptide linker (60-61) to human interleukin 2 (IL-2) (4-74)-peptide (62-132) and via a GSG3S peptide linker (133-138) to human interleukin 2 receptor α-chain (IL2R subunit alpha, IL2Rα, IL2RA) (1-165)-peptide (139-303), produced in Chinese hamster ovary (CHO) cells, glycoform alfa. The amino acid sequence of nemvaleukin alfa is given in SEQ ID NO:6. In some embodiments, nemvaleukin alfa exhibits the following post-translational modifications: disulfide bridges at positions: 31-116, 141-285, 184-242, 269-301, 166-197 or 166-199, 168-199 or 168-197 (using the numbering in SEQ ID NO:6), and glycosylation sites at positions: N187, N206, T212 using the numbering in SEQ ID NO:6. The preparation and properties of nemvaleukin alfa, as well as additional alternative forms of IL-2 suitable for use in the invention, is described in U.S. Patent Application Publication No. US 2021/0038684 A1 and U.S. Pat. No. 10,183,979, the disclosures of which are incorporated by reference herein. In some embodiments, an IL-2 form suitable for use in the invention is a protein having at least 80%, at least 90%, at least 95%, or at least 90% sequence identity to SEQ ID NO:6. In some embodiments, an IL-2 form suitable for use in the invention has the amino acid sequence given in SEQ ID NO:6 or conservative amino acid substitutions thereof. In some embodiments, an IL-2 form suitable for use in the invention is a fusion protein comprising amino acids 24-452 of SEQ ID NO:7, or variants, fragments, or derivatives thereof. In some embodiments, an IL-2 form suitable for use in the invention is a fusion protein comprising an amino acid sequence having at least 80%, at least 90%, at least 95%, or at least 90% sequence identity to amino acids 24-452 of SEQ ID NO:7, or variants, fragments, or derivatives thereof. Other IL-2 forms suitable for use in the present invention are described in U.S. Pat. No. 10,183,979, the disclosures of which are incorporated by reference herein. Optionally, in some embodiments, an IL-2 form suitable for use in the invention is a fusion protein comprising a first fusion partner that is linked to a second fusion partner by a mucin domain polypeptide linker, wherein the first fusion partner is IL-1Rα or a protein having at least 98% amino acid sequence identity to IL-1Rα and having the receptor antagonist activity of IL-Rα, and wherein the second fusion partner comprises all or a portion of an immunoglobulin comprising an Fc region, wherein the mucin domain polypeptide linker comprises SEQ ID NO:8 or an amino acid sequence having at least 90% sequence identity to SEQ ID NO:8 and wherein the half-life of the fusion protein is improved as compared to a fusion of the first fusion partner to the second fusion partner in the absence of the mucin domain polypeptide linker.

TABLE 2
Amino acid sequences of interleukins.

| Identifier | Sequence (One-Letter Amino Acid Symbols) | |
|---|---|---|
| SEQ ID NO: 3 | MAPTSSSTKK TQLQLEHLLL DLQMILNGIN NYKNPKLTRM LTFKFYMPKK ATELKHLQCL | 60 |
| recombinant | EEELKPLEEV LNLAQSKNFH LRPRDLISNI NVIVLELKGS ETTFMCEYAD ETATIVEFLN | 120 |
| human IL-2 | RWITFCQSII STLT | 134 |
| (rhIL-2) | | |
| SEQ ID NO: 4 | PTSSSTKKTQ LQLEHLLLDL QMILNGINNY KNPKLTRMLT FKFYMPKKAT ELKHLQCLEE | 60 |
| Aldesleukin | ELKPLEEVLN LAQSKNFHLR PRDLISNINV IVLELKGSET TFMCEYADET ATIVEFLNRW | 120 |
| | ITFSQSIIST LT | 132 |
| SEQ ID NO: 5 | APTSSSTKKT QLQLEHLLLD LQMILNGINN YKNPKLTRML TFKFYMPKKA TELKHLQCLE | 60 |
| IL-2 form | EELKPLEEVL NLAQSKNFHL RPRDLISNIN VIVLELKGSE TTFMCEYADE TATIVEFLNR | 120 |
| | WITFCQSIIS TLT | 133 |
| SEQ ID NO: 6 | SKNFHLRPRD LISNINVIVL ELKGSETTFM CEYADETATI VEFLNRWITF SQSIISTLTG | 60 |
| Nemvaleukin | GSSSTKKTQL QLEHLLLDLQ MILNGINNYK NPKLTRMLTF KFYMPKKATE LKHLQCLEEE | 120 |
| alfa | LKPLEEVLNL AQGSGGGSEL CDDDPPEIPH ATFKAMAYKE GTMLNCECKR GFRRIKSGSL | 180 |
| | YMLCTGNSSH SSWDNQCQCT SSATRNTTKQ VTPQPEEQKE RKTTEMQSPM QPVDQASLPG | 240 |
| | HCREPPPWEN EATERIYHFV VGQMVYYQCV QGYRALHRGP AESVCKMTHG KTRWTQPQLI | 300 |
| | CTG | 303 |
| SEQ ID NO: 7 | MDAMKRGLCC VLLLCGAVFV SARRPSGRKS SKMQAFRIWD VNQKTFYLRN NQLVAGYLQG | 60 |
| IL-2 form | PNVNLEEKID VVPIEPHALF LGIHGGKMCL SCVKSGDETR LQLEAVNITD LSENRKQDKR | 120 |
| | FAFIRSDSGP TTSFESAACP GWFLCTAMEA DQPVSLTNMP DEGVMVTKFY FQEDESGSGG | 180 |
| | ASSESSASSD GPHPVITESR ASSESSASSD GPHPVITESR EPKSSDKTHT CPPCPAPELL | 240 |
| | GGPSVFLFPP KPKDTLMISR TPEVTCVVVD VSHEDPEVKF NWYVDGVEVH NAKTKPREEQ | 300 |
| | YNSTYRVVSV LTVLHQDWLN GKEYKCKVSN KALPAPIEKT ISKAKGQPRE PQVYTLPPSR | 360 |
| | EEMTKNQVSL TCLVKGFYPS DIAVEWESNG QPENNYKTTP PVLDSDGSFF LYSKLTVDKS | 420 |
| | RWQQGNVFSC SVMHEALHNH YTQKSLSLSP GK | 452 |
| SEQ ID NO: 8 | SESSASSDGP HPVITP | 16 |
| mucin domain | | |
| polypeptide | | |
| SEQ ID NO: 9 | MHKCDITLQE IIKTINSLTE QKTLCTELTV TDIFAASKNT TEKETFCRAA TVLRQFYSHH | 60 |
| recombinant | EKDTRCLGAT AQQFHRHKQL IRFLKRLDRN LWGLAGLNSC PVKEANQSTL ENFLERLKTI | 120 |
| human IL-4 | MREKYSKCSS | 130 |
| (rhIL-4) | | |
| SEQ ID NO: 10 | MDCDIEGKDG KQYESVLMVS IDQLLDSMKE IGSNCLNNEF NFFKRHICDA NKEGMFLFRA | 60 |
| recombinant | ARKLRQFLKM NSTGDFDLHL LKVSEGTTIL LNCTGQVKGR KPAALGEAQP TKSLEENKSL | 120 |
| human IL-7 | KEQKKLNDLC FLKRLLQEIK TCWNKILMGT KEH | 153 |
| (rhIL-7) | | |
| SEQ ID NO: 11 | MNWVNVISDL KKIEDLIQSM HIDATLYTES DVHPSCKVTA MKCFLLELQV ISLESGDASI | 60 |
| recombinant | HDTVENLIIL ANNSLSSNGN VTESGCKECE ELEEKNIKEF LQSFVHIVQM FINTS | 115 |
| human IL-15 | | |
| (rhIL-15) | | |
| SEQ ID NO: 12 | MQDRHMIRMR QLIDIVDQLK NYVNDLVPEF LPAPEDVETN CEWSAFSCFQ KAQLKSANTG | 60 |
| recombinant | NNERIINVSI KKLKRKPPST NAGRRQKHRL TCPSCDSYEK KPPKEFLERF KSLLQKMIHQ | 120 |
| human IL-21 | HLSSRTHGSE DS | 132 |
| (rhIL-21) | | |

In some embodiments, an IL-2 form suitable for use in the invention includes a antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the antibody cytokine engrafted protein preferentially expands T effector cells over regulatory T cells. In some embodiments, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the IL-2 molecule is a mutein, and wherein the antibody cytokine engrafted protein preferentially expands T effector cells over regulatory T cells. In some embodiments, the IL-2 regimen comprises administration of an antibody described in U.S. Patent Application Publication No. US 2020/0270334 A1, the disclosures of which are incorporated by reference herein. In some embodiments, the antibody cytokine engrafted protein comprises a heavy chain variable region (VH), comprising complementarity determining regions HCDR1, HCDR2, HCDR3; a light chain variable region (VL), comprising LCDR1, LCDR2, LCDR3; and an IL-2 molecule or a fragment thereof engrafted into a CDR of the V_{H} or the V_{L}, wherein the IL-2 molecule is a mutein, wherein the antibody cytokine engrafted protein preferentially expands T effector cells over regulatory T cells, and wherein the antibody further comprises an IgG class heavy chain and an IgG class light chain selected from the group consisting of: a IgG class light chain comprising SEQ ID NO:39 and a IgG class heavy chain comprising SEQ ID NO:38; a IgG class light chain comprising SEQ ID NO:37 and a IgG class heavy chain comprising SEQ ID NO:29; a IgG class light chain comprising SEQ ID NO:39 and a IgG class heavy chain comprising SEQ ID NO:29; and a IgG class light chain comprising SEQ ID NO:37 and a IgG class heavy chain comprising SEQ ID NO:38.

In some embodiments, an IL-2 molecule or a fragment thereof is engrafted into HCDR1 of the V_{H}, wherein the IL-2 molecule is a mutein. In some embodiments, an IL-2 molecule or a fragment thereof is engrafted into HCDR2 of the V_{H}, wherein the IL-2 molecule is a mutein. In some embodiments, an IL-2 molecule or a fragment thereof is engrafted into HCDR3 of the V_{H}, wherein the IL-2 molecule is a mutein. In some embodiments, an IL-2 molecule or a fragment thereof is engrafted into LCDR1 of the V_{L}, wherein the IL-2 molecule is a mutein. In some embodiments, an IL-2 molecule or a fragment thereof is engrafted into LCDR2 of the V_{L}, wherein the IL-2 molecule is a mutein. In some embodiments, an IL-2 molecule or a fragment thereof is engrafted into LCDR3 of the V_{L}, wherein the IL-2 molecule is a mutein.

The insertion of the IL-2 molecule can be at or near the N-terminal region of the CDR, in the middle region of the CDR or at or near the C-terminal region of the CDR. In some embodiments, the antibody cytokine engrafted protein comprises an IL-2 molecule incorporated into a CDR, wherein the IL2 sequence does not frameshift the CDR sequence. In some embodiments, the antibody cytokine engrafted protein comprises an IL-2 molecule incorporated into a CDR, wherein the IL-2 sequence replaces all or part of a CDR sequence. The replacement by the IL-2 molecule can be the N-terminal region of the CDR, in the middle region of the CDR or at or near the C-terminal region the CDR. A replacement by the IL-2 molecule can be as few as one or two amino acids of a CDR sequence, or the entire CDR sequences.

In some embodiments, an IL-2 molecule is engrafted directly into a CDR without a peptide linker, with no additional amino acids between the CDR sequence and the IL-2 sequence. In some embodiments, an IL-2 molecule is engrafted indirectly into a CDR with a peptide linker, with one or more additional amino acids between the CDR sequence and the IL-2 sequence.

In some embodiments, the IL-2 molecule described herein is an IL-2 mutein. In some instances, the IL-2 mutein comprising an R67A substitution. In some embodiments, the IL-2 mutein comprises the amino acid sequence SEQ ID NO:14 or SEQ ID NO:15. In some embodiments, the IL-2 mutein comprises an amino acid sequence in Table 1 in U.S. Patent Application Publication No. US 2020/0270334 A1, the disclosure of which is incorporated by reference herein.

In some embodiments, the antibody cytokine engrafted protein comprises an HCDR1 selected from the group consisting of SEQ ID NO:16, SEQ ID NO:19, SEQ ID NO:22 and SEQ ID NO:25. In some embodiments, the antibody cytokine engrafted protein comprises an HCDR1 selected from the group consisting of SEQ ID NO:7, SEQ ID NO:10, SEQ ID NO:13 and SEQ ID NO:16. In some embodiments, the antibody cytokine engrafted protein comprises an HCDR1 selected from the group consisting of HCDR2 selected from the group consisting of SEQ ID NO:17, SEQ ID NO:20, SEQ ID NO:23, and SEQ ID NO:26. In some embodiments, the antibody cytokine engrafted protein comprises an HCDR3 selected from the group consisting of SEQ ID NO:18, SEQ ID NO:21, SEQ ID NO:24, and SEQ ID NO:27. In some embodiments, the antibody cytokine engrafted protein comprises a V_{H} region comprising the amino acid sequence of SEQ ID NO:28. In some embodiments, the antibody cytokine engrafted protein comprises a heavy chain comprising the amino acid sequence of SEQ ID NO:29. In some embodiments, the antibody cytokine engrafted protein comprises a V_{L} region comprising the amino acid sequence of SEQ ID NO:36. In some embodiments, the antibody cytokine engrafted protein comprises a light chain comprising the amino acid sequence of SEQ ID NO:37. In some embodiments, the antibody cytokine engrafted protein comprises a V_{H} region comprising the amino acid sequence of SEQ ID NO:28 and a V_{L} region comprising the amino acid sequence of SEQ ID NO:36. In some embodiments, the antibody cytokine engrafted protein comprises a heavy chain region comprising the amino acid sequence of SEQ ID NO:29 and a light chain region comprising the amino acid sequence of SEQ ID NO:37. In some embodiments, the antibody cytokine engrafted protein comprises a heavy chain region comprising the amino acid sequence of SEQ ID NO:29 and a light chain region comprising the amino acid sequence of SEQ ID NO:39. In some embodiments, the antibody cytokine engrafted protein comprises a heavy chain region comprising the amino acid sequence of SEQ ID NO:38 and a light chain region comprising the amino acid sequence of SEQ ID NO:37. In some embodiments, the antibody cytokine engrafted protein comprises a heavy chain region comprising the amino acid sequence of SEQ ID NO:38 and a light chain region comprising the amino acid sequence of SEQ ID NO:39. In some embodiments, the antibody cytokine engrafted protein comprises IgG.IL2F71A.H1 or IgG.IL2R67A.H1 of U.S. Patent Application Publication No. 2020/0270334 A1, or variants, derivatives, or fragments thereof, or conservative amino acid substitutions thereof, or proteins with at least 80%, at least 90%, at least 95%, or at least 98% sequence identity thereto. In some embodiments, the antibody components of the antibody cytokine engrafted protein described herein comprise immunoglobulin sequences, framework sequences, or CDR sequences of palivizumab. In some embodiments, the antibody cytokine engrafted protein described herein has a longer serum half-life than a wild-type IL-2 molecule such as, but not limited to, aldesleukin or a comparable molecule. In some embodiments, the antibody cytokine engrafted protein described herein has a sequence as set forth in Table 3.

TABLE 3
Sequences of exemplary palivizumab antibody-IL-2 engrafted proteins

| Identifier | Sequence (One-Letter Amino Acid Symbols) | |
|---|---|---|
| SEQ ID NO: 13 | MYRMQLLSCI ALSLALVTNS APTSSSTKKT QLQLEHLLLD LQMILNGINN YKNPKLTRML | 60 |
| IL-2 | TFKFYMPKKA TELKHLQCLE EELKPLEEVL NLAQSKNFHL RPRDLISNIN VIVLELKGSE | 120 |
| | TTFMCEYADE TATIVEFLNR WITFCQSIIS TLT | 153 |
| SEQ ID NO: 14 | APTSSSTKKT QLQLEHLLLD LQMILNGINN YKNPKLTAML TFKFYMPKKA TELKHLQCLE | 60 |
| IL-2 mutein | EELKPLEEVL NLAQSKNFHL RPRDLISNIN VIVLELKGSE TTFMCEYADE TATIVEFLNR | 120 |
| | WITFCQSIIS TLT | 133 |
| SEQ ID NO: 15 | APTSSSTKKT QLQLEHLLLD LQMILNGINN YKNPKLTRML TAKFYMPKKA TELKHLQCLE | 60 |
| IL-2 mutein | EELKPLEEVL NLAQSKNFHL RPRDLISNIN VIVLELKGSE TTFMCEYADE TATIVEFLNR | 120 |
| | WITFCQSIIS TLT | 133 |
| SEQ ID NO: 16 | GFSLAPTSSS TKKTQLQLEH LLLDLQMILN GINNYKNPKL TAMLTFKFYM PKKATELKHL | 60 |
| HCDR1 IL-2 | QCLEEELKPL EEVLNLAQSK NFHLRPRDLI SNINVIVLEL KGSETTFMCE YADETATIVE | 120 |
| | FLNRWITFCQ SIISTLTSTS GMSVG | 145 |
| SEQ ID NO: 17 | DIWWDDKKDY NPSLKS | 16 |
| HCDR2 | | |
| SEQ ID NO: 18 | SMITNWYFDV | 10 |
| HCDR3 | | |
| SEQ ID NO: 19 | APTSSSTKKT QLQLEHLLLD LQMILNGINN YKNPKLTAML TFKFYMPKKA TELKHLQCLE | 60 |
| HCDR1 IL-2 | EELKPLEEVL NLAQSKNFHL RPRDLISNIN VIVLELKGSE TTFMCEYADE TATIVEFLNR | 120 |
| kabat | WITFCQSIIS TLTSTSGMSV G | 141 |
| SEQ ID NO: 20 | DIWWDDKKDY NPSLKS | 16 |
| HCDR2 kabat | | |
| SEQ ID NO: 21 | SMITNWYFDV | 10 |
| HCDR3 kabat | | |
| SEQ ID NO: 22 | GFSLAPTSSS TKKTQLQLEH LLLDLQMILN GINNYKNPKL TAMLTFKFYM PKKATELKHL | 60 |
| HCDR1 IL-2 | QCLEEELKPL EEVLNLAQSK NFHLRPRDLI SNINVIVLEL KGSETTFMCE YADETATIVE | 120 |
| clothia | FLNRWITFCQ SIISTLTSTS GM | 142 |
| SEQ ID NO: 23 | WWDDK | 5 |
| HCDR2 clothia | | |
| SEQ ID NO: 24 | SMITNWYFDV | 10 |
| HCDR3 clothia | | |
| SEQ ID NO: 25 | GFSLAPTSSS TKKTQLQLEH LLLDLQMILN GINNYKNPKL TAMLTFKFYM PKKATELKHL | 60 |
| HCDR1 IL-2 IMGT | QCLEEELKPL EEVLNLAQSK NFHLRPRDLI SNINVIVLEL KGSETTFMCE YADETATIVE | 120 |
| | FLNRWITFCQ SIISTLTSTS GMS | 143 |
| SEQ ID NO: 26 | IWWDDKK | 7 |
| HCDR2 IMGT | | |
| SEQ ID NO: 27 | ARSMITNWYF DV | 12 |
| HCDR3 IMGT | | |
| SEQ ID NO: 28 | QVTLRESGPA LVKPTQTLTL TCTFSGFSLA PTSSSTKKTQ LQLEHLLLDL QMILNGINNY | 60 |
| VH | KNPKLTAMLT FKFYMPKKAT ELKHLQCLEE ELKPLEEVLN LAQSKNFHLR PRDLISNINV | 120 |
| | IVLELKGSET TFMCEYADET ATIVEFLNRW ITFCQSIIST LTSTSGMSVG WIRQPPGKAL | 180 |
| | EWLADIWWDD KKDYNPSLKS RLTISKDTSK NQVVLKVTNM DPADTATYYC ARSMITNWYF | 240 |
| | DVWGAGTTVT VSS | 253 |
| SEQ ID NO: 29 | QMILNGINNY KNPKLTAMLT FKFYMPKKAT ELKHLQCLEE ELKPLEEVLN LAQSKNFHLR | 60 |
| Heavy chain | PRDLISNINV IVLELKGSET TFMCEYADET ATIVEFLNRW ITFCQSIIST LTSTSGMSVG | 120 |
| | WIRQPPGKAL EWLADIWWDD KKDYNPSLKS RLTISKDTSK NQVVLKVTNM DPADTATYYC | 180 |
| | ARSMITNWYF DVWGAGTTVT VSSASTKGPS VFPLAPSSKS TSGGTAALGC LVKDYFPEPV | 240 |
| | TVSWNSGALT SGVHTFPAVL QSSGLYSLSS VVTVPSSSLG TQTYICNVNH KPSNTKVDKR | 300 |
| | VEPKSCDKTH TCPPCPAPEL LGGPSVFLFP PKPKDTLMIS RTPEVTCVVV AVSHEDPEVK | 360 |
| | FNWYVDGVEV HNAKTKPREE QYNSTYRVVS VLTVLHQDWL NGKEYKCKVS NKALAAPIEK | 420 |
| | TISKAKGQPR EPQVYTLPPS REEMTKNQVS LTCLVKGFYP SDIAVEWESN GQPENNYKTT | 480 |
| | PPVLDSDGSF FLYSKLTVDK SRWQQGNVFS CSVMHEALHN HYTQKSLSLS PGK | 533 |
| SEQ ID NO: 30 | KAQLSVGYMH | 10 |
| LCDR1 kabat | | |
| SEQ ID NO: 31 | DTSKLAS | 7 |
| LCDR2 kabat | | |
| SEQ ID NO: 32 | FQGSGYPFT | 9 |
| LCDR3 kabat | | |
| SEQ ID NO: 33 | QLSVGY | 6 |
| LCDR1 chothia | | |
| SEQ ID NO: 34 | DTS | 3 |
| LCDR2 chothia | | |
| SEQ ID NO: 35 | GSGYPF | 6 |
| LCDR3 chothia | | |
| SEQ ID NO: 36 | DIQMTQSPST LSASVGDRVT ITCKAQLSVG YMHWYQQKPG KAPKLLIYDT SKLASGVPSR | 60 |
| VL | FSGSGSGTEF TLTISSLQPD DFATYYCFQG SGYPFTFGGG TKLEIK | 106 |
| SEQ ID NO: 37 | DIQMTQSPST LSASVGDRVT ITCKAQLSVG YMHWYQQKPG KAPKLLIYDT SKLASGVPSR | 60 |
| Light chain | FSGSGSGTEF TLTISSLQPD DFATYYCFQG SGYPFTFGGG TKLEIKRTVA APSVFIFPPS | 120 |
| | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |
| SEQ ID NO: 38 | QVTLRESGPA LVKPTQTLTL TCTFSGFSLA PTSSSTKKTQ LQLEHLLLDL QMILNGINNY | 60 |
| Light chain | KNPKLTRMLT AKFYMPKKAT ELKHLQCLEE ELKPLEEVLN LAQSKNFHLR PRDLISNINV | 120 |
| | IVLELKGSET TFMCEYADET ATIVEFLNRW ITFCQSIIST LTSTSGMSVG WIRQPPGKAL | 180 |
| | EWLADIWWDD KKDYNPSLKS RLTISKDTSK NQVVLKVTNM DPADTATYYC ARSMITNWYF | 240 |
| | DVWGAGTTVT VSSASTKGPS VFPLAPSSKS TSGGTAALGC LVKDYFPEPV TVSWNSGALT | 300 |
| | SGVHTFPAVL QSSGLYSLSS VVTVPSSSLG TQTYICNVNH KPSNTKVDKR VEPKSCDKTH | 360 |
| | TCPPCPAPEL LGGPSVFLFP PKPKDTLMIS RTPEVTCVVV AVSHEDPEVK FNWYVDGVEV | 420 |
| | HNAKTKPREE QYNSTYRVVS VLTVLHQDWL NGKEYKCKVS NKALAAPIEK TISKAKGQPR | 480 |
| | EPQVYTLPPS REEMTKNQVS LTCLVKGFYP SDIAVEWESN GQPENNYKTT PPVLDSDGSF | 540 |
| | FLYSKLTVDK SRWQQGNVFS CSVMHEALHN HYTQKSLSLS PGK | 583 |
| SEQ ID NO: 39 | DIQMTQSPST LSASVGDRVT ITCKAQLSVG YMHWYQQKPG KAPKLLIYDT SKLASGVPSR | 60 |
| Light chain | FSGSGSGTEF TLTISSLQPD DFATYYCFQG SGYPFTFGGG TKLEIKRTVA APSVFIFPPS | 120 |
| | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSFNR GEC | 213 |

The term “IL-4” (also referred to herein as “IL4”) refers to the cytokine known as interleukin 4, which is produced by Th2 T cells and by eosinophils, basophils, and mast cells. IL-4 regulates the differentiation of naïve helper T cells (Th0 cells) to Th2 T cells. Steinke and Borish, Respir. Res. 2001, 2, 66-70. Upon activation by IL-4, Th2 T cells subsequently produce additional IL-4 in a positive feedback loop. IL-4 also stimulates B cell proliferation and class II MHC expression, and induces class switching to IgE and IgG₁ expression from B cells. Recombinant human IL-4 suitable for use in the invention is commercially available from multiple suppliers, including ProSpec-Tany TechnoGene Ltd., East Brunswick, NJ, USA (Cat. No. CYT-211) and ThermoFisher Scientific, Inc., Waltham, MA, USA (human IL-15 recombinant protein, Cat. No. Gibco CTP0043). The amino acid sequence of recombinant human IL-4 suitable for use in the invention is given in Table 2 (SEQ ID NO:9).

The term “IL-7” (also referred to herein as “IL7”) refers to a glycosylated tissue-derived cytokine known as interleukin 7, which may be obtained from stromal and epithelial cells, as well as from dendritic cells. Fry and Mackall, Blood 2002, 99, 3892-904. IL-7 can stimulate the development of T cells. IL-7 binds to the IL-7 receptor, a heterodimer consisting of IL-7 receptor alpha and common gamma chain receptor, which in a series of signals important for T cell development within the thymus and survival within the periphery. Recombinant human IL-7 suitable for use in the invention is commercially available from multiple suppliers, including ProSpec-Tany TechnoGene Ltd., East Brunswick, NJ, USA (Cat. No. CYT-254) and ThermoFisher Scientific, Inc., Waltham, MA, USA (human IL-15 recombinant protein, Cat. No. Gibco PHC0071). The amino acid sequence of recombinant human IL-7 suitable for use in the invention is given in Table 2 (SEQ ID NO:10).

The term “IL-15” (also referred to herein as “IL15”) refers to the T cell growth factor known as interleukin-15, and includes all forms of IL-2 including human and mammalian forms, conservative amino acid substitutions, glycoforms, biosimilars, and variants thereof. IL-15 is described, e.g., in Fehniger and Caligiuri, Blood 2001, 97, 14-32, the disclosure of which is incorporated by reference herein. IL-15 shares β and γ signaling receptor subunits with IL-2. Recombinant human IL-15 is a single, non-glycosylated polypeptide chain containing 114 amino acids (and an N-terminal methionine) with a molecular mass of 12.8 kDa. Recombinant human IL-15 is commercially available from multiple suppliers, including ProSpec-Tany TechnoGene Ltd., East Brunswick, NJ, USA (Cat. No. CYT-230-b) and ThermoFisher Scientific, Inc., Waltham, MA, USA (human IL-15 recombinant protein, Cat. No. 34-8159-82). The amino acid sequence of recombinant human IL-15 suitable for use in the invention is given in Table 2 (SEQ ID NO:11).

The term “IL-21” (also referred to herein as “IL21”) refers to the pleiotropic cytokine protein known as interleukin-21, and includes all forms of IL-21 including human and mammalian forms, conservative amino acid substitutions, glycoforms, biosimilars, and variants thereof. IL-21 is described, e.g., in Spolski and Leonard, Nat. Rev. Drug. Disc. 2014, 13, 379-95, the disclosure of which is incorporated by reference herein. IL-21 is primarily produced by natural killer T cells and activated human CD4+ T cells. Recombinant human IL-21 is a single, non-glycosylated polypeptide chain containing 132 amino acids with a molecular mass of 15.4 kDa. Recombinant human IL-21 is commercially available from multiple suppliers, including ProSpec-Tany TechnoGene Ltd., East Brunswick, NJ, USA (Cat. No. CYT-408-b) and ThermoFisher Scientific, Inc., Waltham, MA, USA (human IL-21 recombinant protein, Cat. No. 14-8219-80). The amino acid sequence of recombinant human IL-21 suitable for use in the invention is given in Table 2 (SEQ ID NO:12).

When “an anti-tumor effective amount”, “a tumor-inhibiting effective amount”, or “therapeutic amount” is indicated, the precise amount of the compositions of the present invention to be administered can be determined by a physician with consideration of individual differences in age, weight, tumor size, extent of infection or metastasis, and condition of the patient (subject). It can generally be stated that a pharmaceutical composition comprising the tumor infiltrating lymphocytes (e.g. secondary TILs or genetically modified cytotoxic lymphocytes) described herein may be administered at a dosage of 10⁴ to 10¹¹ cells/kg body weight (e.g., 10⁵ to 10⁶, 10⁵ to 10¹⁰, 10⁵ to 10¹¹, 10⁶ to 10¹⁰, 10⁶ to 10¹¹, 10⁷ to 10¹¹, 10⁷ to 10¹⁰, 10⁸ to 10¹¹, 10⁸ to 10¹⁰, 10⁹ to 10¹¹, or 10⁹ to 10¹⁰ cells/kg body weight), including all integer values within those ranges. TILs (including in some cases, genetically modified cytotoxic lymphocytes) compositions may also be administered multiple times at these dosages. The TILs (including, in some cases, genetically engineered TILs) can be administered by using infusion techniques that are commonly known in immunotherapy (see, e.g., Rosenberg, et al., New Eng. J. of Med. 1988, 319, 1676). The optimal dosage and treatment regime for a particular patient can readily be determined by one skilled in the art of medicine by monitoring the patient for signs of disease and adjusting the treatment accordingly.

The term “hematological malignancy”, “hematologic malignancy” or terms of correlative meaning refer to mammalian cancers and tumors of the hematopoietic and lymphoid tissues, including but not limited to tissues of the blood, bone marrow, lymph nodes, and lymphatic system. Hematological malignancies are also referred to as “liquid tumors.” Hematological malignancies include, but are not limited to, acute lymphoblastic leukemia (ALL), chronic lymphocytic lymphoma (CLL), small lymphocytic lymphoma (SLL), acute myelogenous leukemia (AML), chronic myelogenous leukemia (CML), multiple myeloma, acute monocytic leukemia (AMoL), Hodgkin's lymphoma, and non-Hodgkin's lymphomas. The term “B cell hematological malignancy” refers to hematological malignancies that affect B cells.

The term “liquid tumor” refers to an abnormal mass of cells that is fluid in nature. Liquid tumor cancers include, but are not limited to, leukemias, myelomas, and lymphomas, as well as other hematological malignancies. TILs obtained from liquid tumors may also be referred to herein as marrow infiltrating lymphocytes (MILs). TILs obtained from liquid tumors, including liquid tumors circulating in peripheral blood, may also be referred to herein as PBLs. The terms MIL, TIL, and PBL are used interchangeably herein and differ only based on the tissue type from which the cells are derived.

The term “microenvironment,” as used herein, may refer to the solid or hematological tumor microenvironment as a whole or to an individual subset of cells within the microenvironment. The tumor microenvironment, as used herein, refers to a complex mixture of “cells, soluble factors, signaling molecules, extracellular matrices, and mechanical cues that promote neoplastic transformation, support tumor growth and invasion, protect the tumor from host immunity, foster therapeutic resistance, and provide niches for dominant metastases to thrive,” as described in Swartz, et al., Cancer Res., 2012, 72, 2473. Although tumors express antigens that should be recognized by T cells, tumor clearance by the immune system is rare because of immune suppression by the microenvironment.

In some embodiments, the invention includes a method of treating a cancer with a population of TILs, wherein a patient is pre-treated with non-myeloablative chemotherapy prior to an infusion of TILs according to the invention. In some embodiments, the population of TILs may be provided wherein a patient is pre-treated with nonmyeloablative chemotherapy prior to an infusion of TILs according to the present invention. In some embodiments, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In some embodiments, after non-myeloablative chemotherapy and TIL infusion (at day 0) according to the invention, the patient receives an intravenous infusion of IL-2 intravenously at 720,000 IU/kg every 8 hours to physiologic tolerance.

Experimental findings indicate that lymphodepletion prior to adoptive transfer of tumor-specific T lymphocytes plays a key role in enhancing treatment efficacy by eliminating regulatory T cells and competing elements of the immune system (“cytokine sinks”). Accordingly, some embodiments of the invention utilize a lymphodepletion step (sometimes also referred to as “immunosuppressive conditioning”) on the patient prior to the introduction of the TILs of the invention.

The term “effective amount” or “therapeutically effective amount” refers to that amount of a compound or combination of compounds as described herein that is sufficient to effect the intended application including, but not limited to, disease treatment. A therapeutically effective amount may vary depending upon the intended application (in vitro or in vivo), or the subject and disease condition being treated (e.g., the weight, age and gender of the subject), the severity of the disease condition, or the manner of administration. The term also applies to a dose that will induce a particular response in target cells (e.g., the reduction of platelet adhesion and/or cell migration). The specific dose will vary depending on the particular compounds chosen, the dosing regimen to be followed, whether the compound is administered in combination with other compounds, timing of administration, the tissue to which it is administered, and the physical delivery system in which the compound is carried.

The terms “treatment”, “treating”, “treat”, and the like, refer to obtaining a desired pharmacologic and/or physiologic effect. The effect may be prophylactic in terms of completely or partially preventing a disease or symptom thereof and/or may be therapeutic in terms of a partial or complete cure for a disease and/or adverse effect attributable to the disease. “Treatment”, as used herein, covers any treatment of a disease in a mammal, particularly in a human, and includes: (a) preventing the disease from occurring in a subject which may be predisposed to the disease but has not yet been diagnosed as having it; (b) inhibiting the disease, i.e., arresting its development or progression; and (c) relieving the disease, i.e., causing regression of the disease and/or relieving one or more disease symptoms. “Treatment” is also meant to encompass delivery of an agent in order to provide for a pharmacologic effect, even in the absence of a disease or condition. For example, “treatment” encompasses delivery of a composition that can elicit an immune response or confer immunity in the absence of a disease condition, e.g., in the case of a vaccine.

The term “heterologous” when used with reference to portions of a nucleic acid or protein indicates that the nucleic acid or protein comprises two or more subsequences that are not found in the same relationship to each other in nature. For instance, the nucleic acid is typically recombinantly produced, having two or more sequences from unrelated genes arranged to make a new functional nucleic acid, e.g., a promoter from one source and a coding region from another source, or coding regions from different sources. Similarly, a heterologous protein indicates that the protein comprises two or more subsequences that are not found in the same relationship to each other in nature (e.g., a fusion protein).

The terms “sequence identity,” “percent identity,” and “sequence percent identity” (or synonyms thereof, e.g., “99% identical”) in the context of two or more nucleic acids or polypeptides, refer to two or more sequences or subsequences that are the same or have a specified percentage of nucleotides or amino acid residues that are the same, when compared and aligned (introducing gaps, if necessary) for maximum correspondence, not considering any conservative amino acid substitutions as part of the sequence identity. The percent identity can be measured using sequence comparison software or algorithms or by visual inspection. Various algorithms and software are known in the art that can be used to obtain alignments of amino acid or nucleotide sequences. Suitable programs to determine percent sequence identity include for example the BLAST suite of programs available from the U.S. Government's National Center for Biotechnology Information BLAST web site. Comparisons between two sequences can be carried using either the BLASTN or BLASTP algorithm. BLASTN is used to compare nucleic acid sequences, while BLASTP is used to compare amino acid sequences. ALIGN, ALIGN-2 (Genentech, South San Francisco, California) or MegAlign, available from DNASTAR, are additional publicly available software programs that can be used to align sequences. One skilled in the art can determine appropriate parameters for maximal alignment by particular alignment software. In certain embodiments, the default parameters of the alignment software are used.

As used herein, the term “variant” encompasses but is not limited to antibodies or fusion proteins which comprise an amino acid sequence which differs from the amino acid sequence of a reference antibody by way of one or more substitutions, deletions and/or additions at certain positions within or adjacent to the amino acid sequence of the reference antibody. The variant may comprise one or more conservative substitutions in its amino acid sequence as compared to the amino acid sequence of a reference antibody. Conservative substitutions may involve, e.g., the substitution of similarly charged or uncharged amino acids. The variant retains the ability to specifically bind to the antigen of the reference antibody. The term variant also includes pegylated antibodies or proteins.

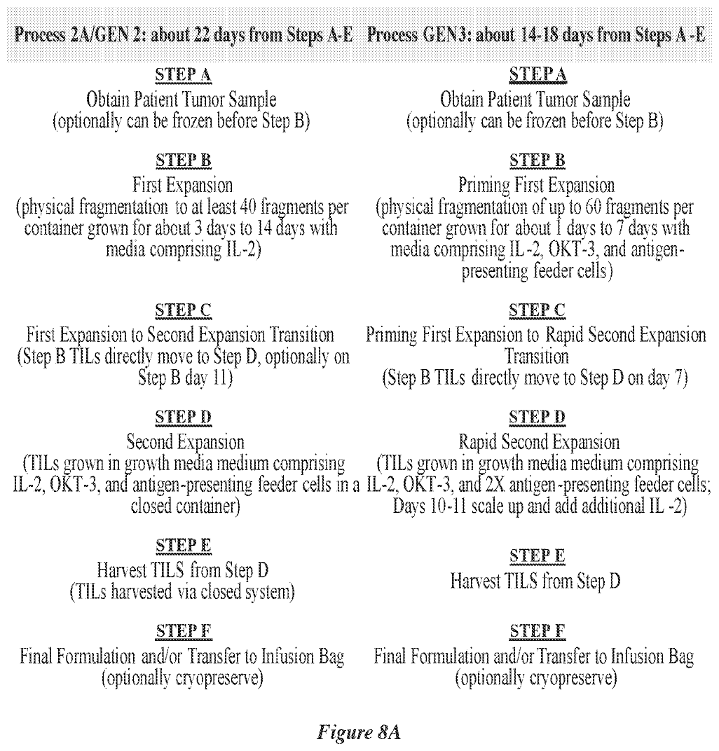

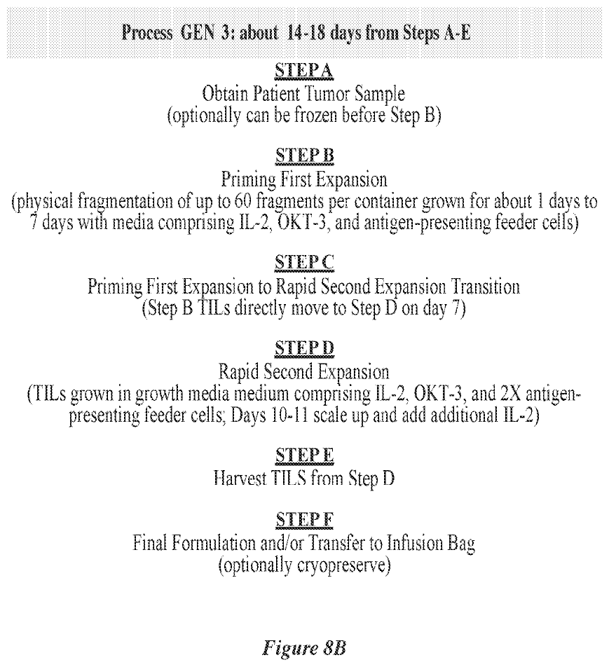

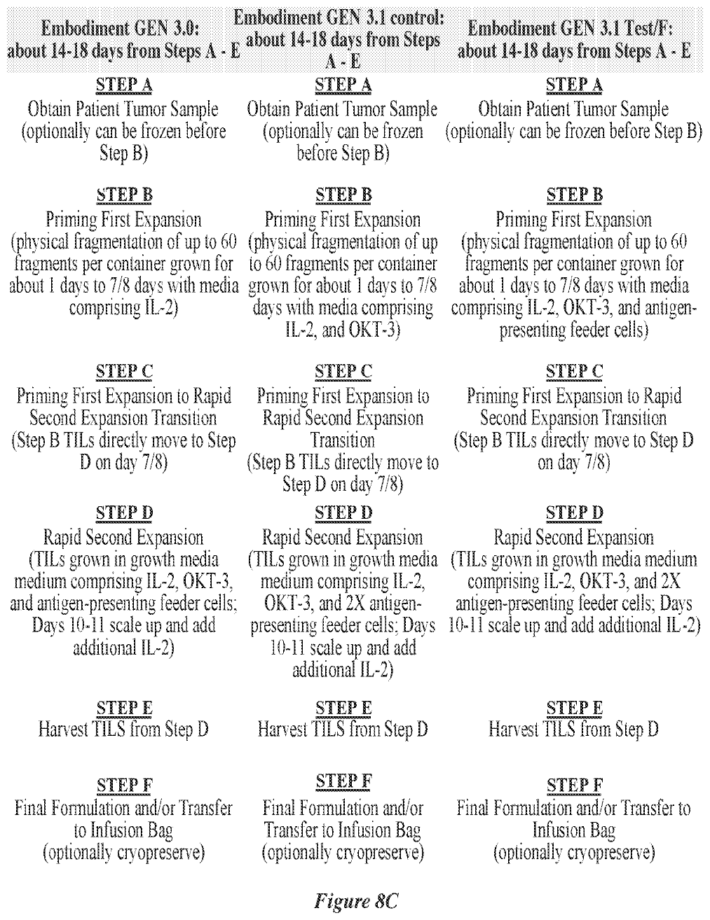

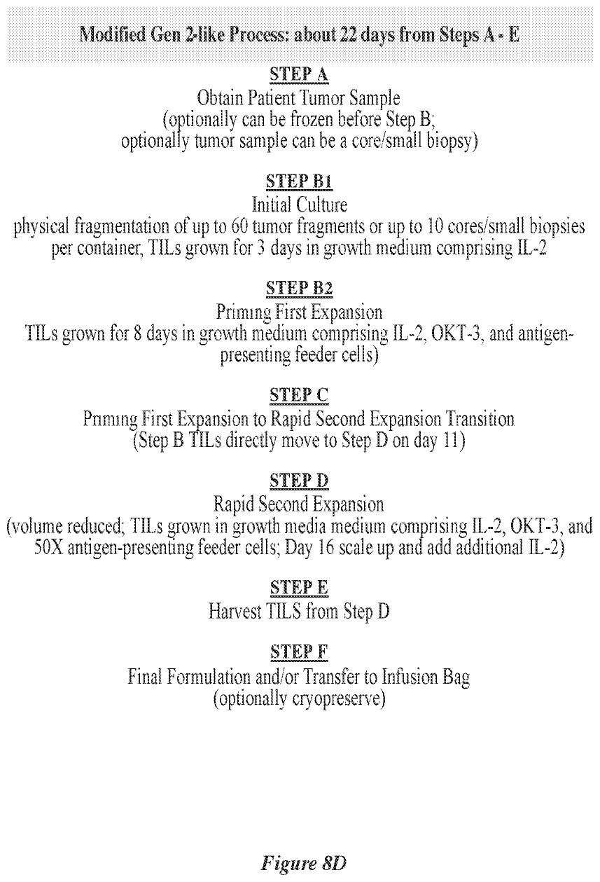

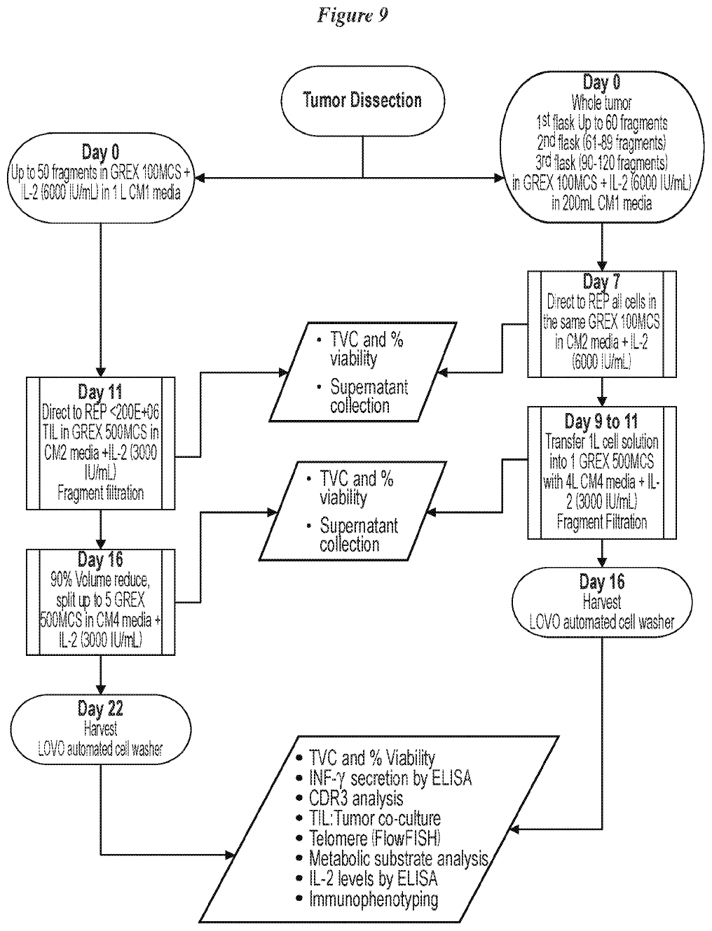

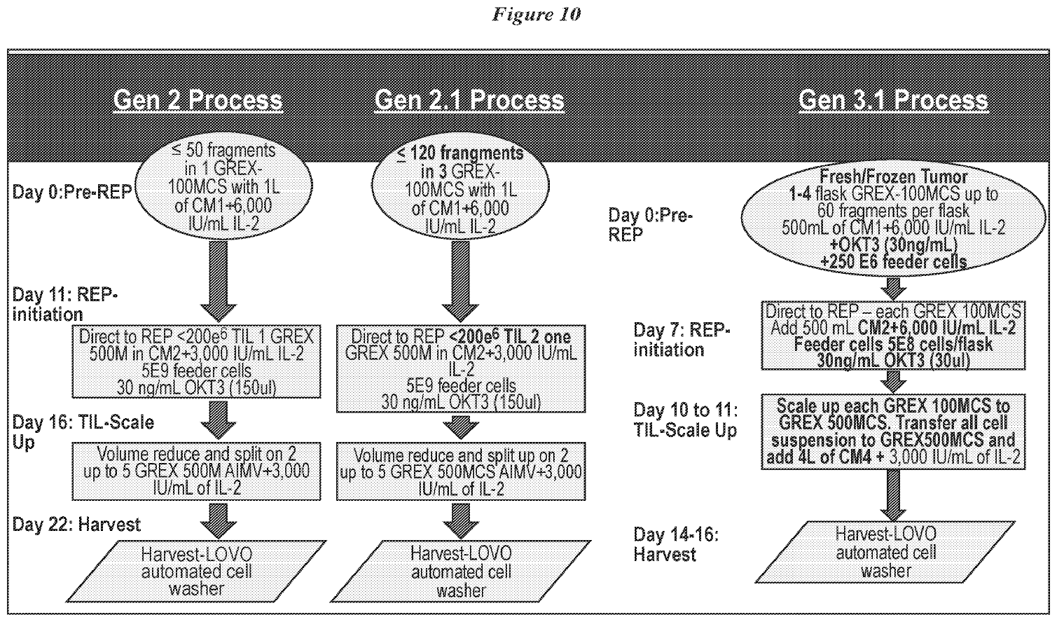

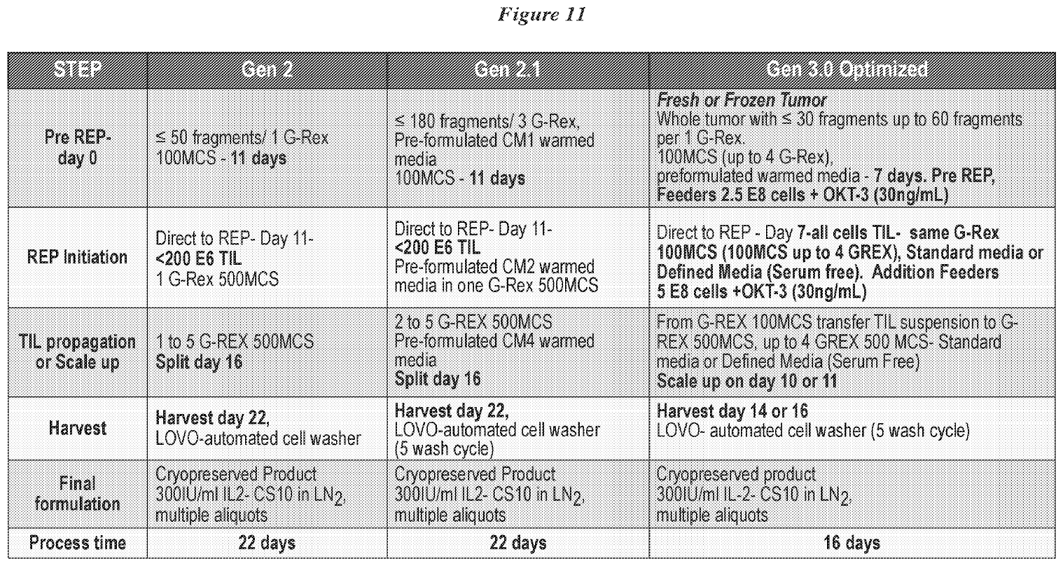

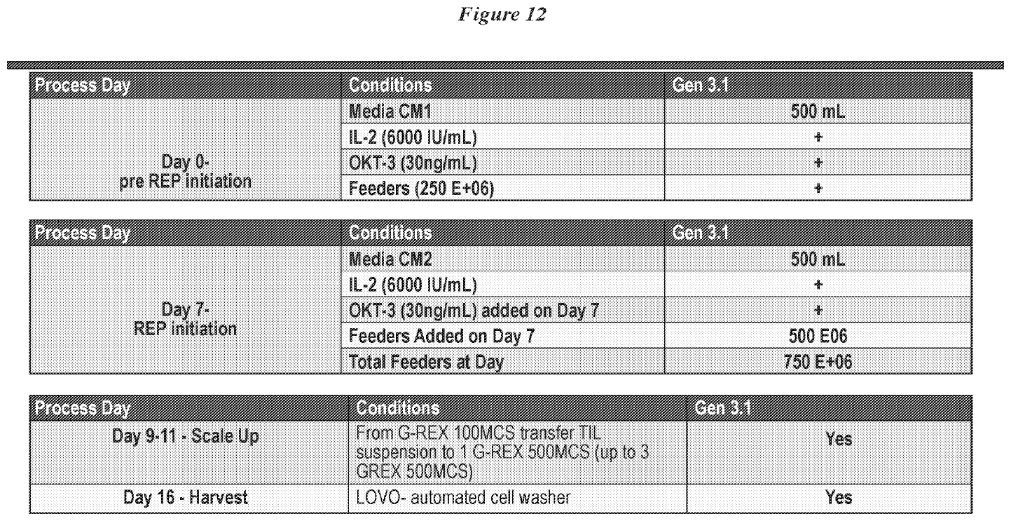

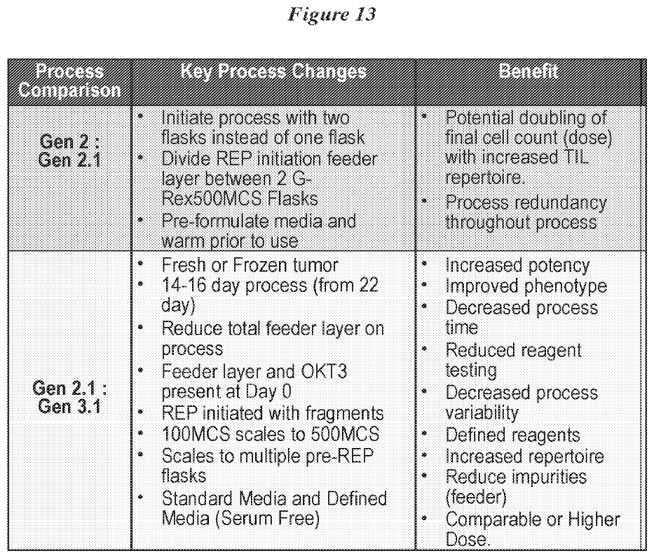

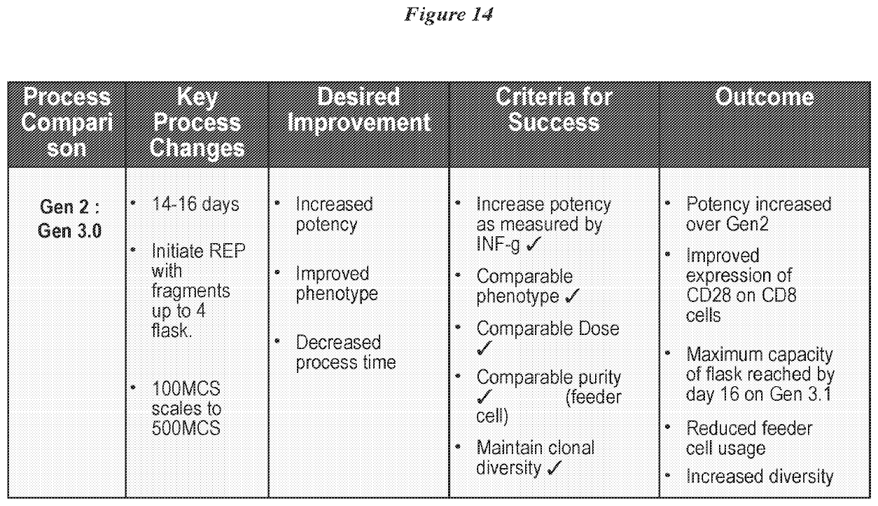

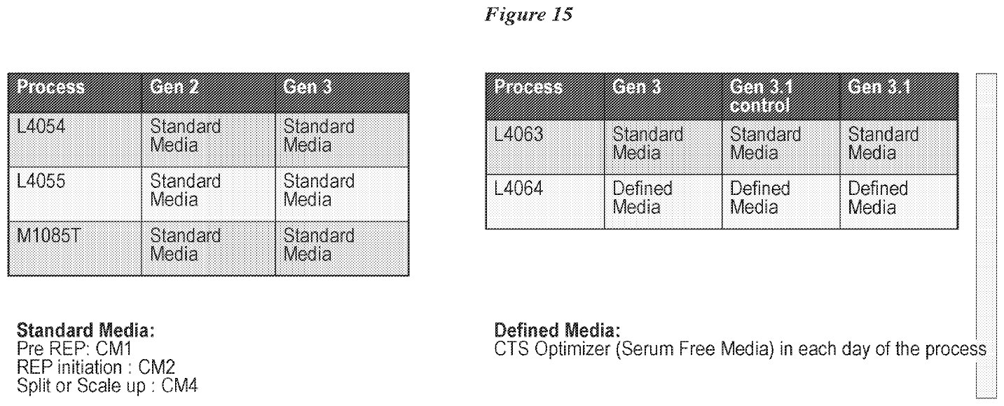

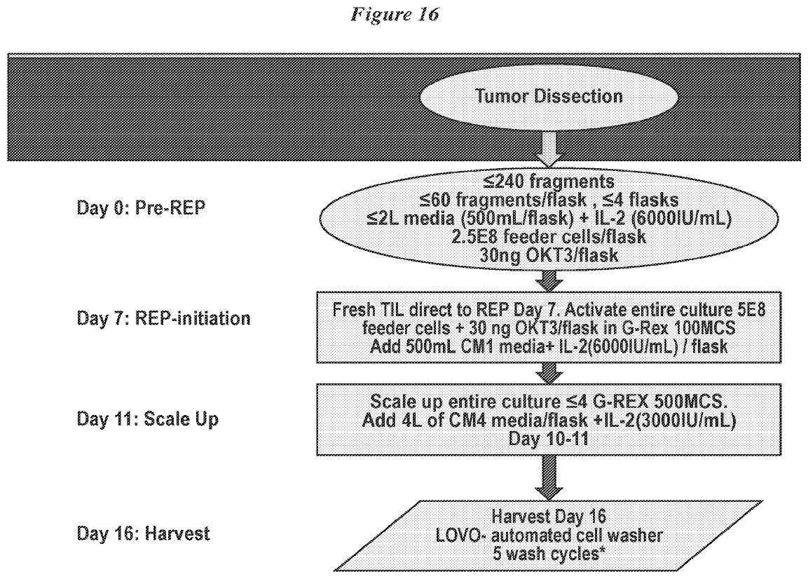

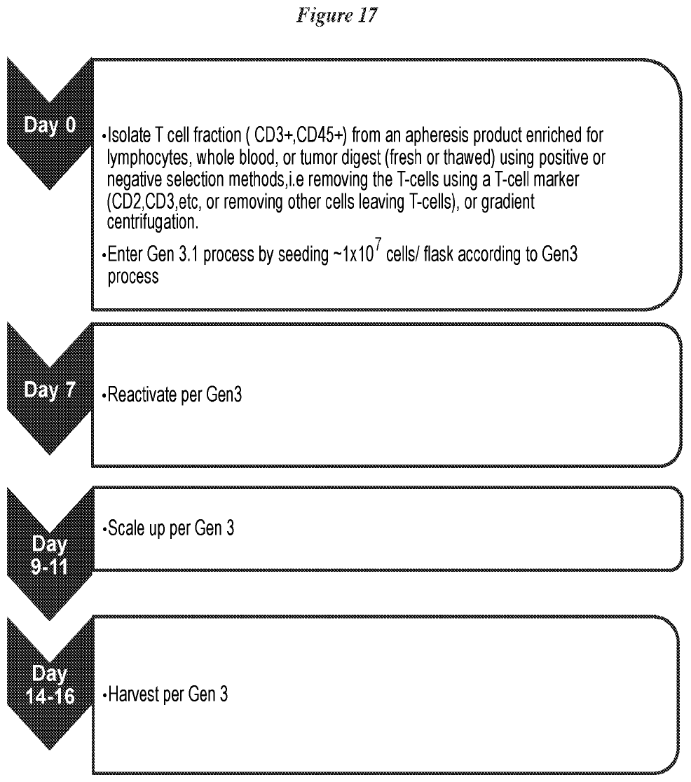

By “tumor infiltrating lymphocytes” or “TILs” herein is meant a population of cells originally obtained as white blood cells that have left the bloodstream of a subject and migrated into a tumor. TILs include, but are not limited to, CD8⁺ cytotoxic T cells (lymphocytes), Th1 and Th17 CD4⁺ T cells, natural killer cells, dendritic cells and M1 macrophages. TILs include both primary and secondary TILs. “Primary TILs” are those that are obtained from patient tissue samples as outlined herein (sometimes referred to as “freshly harvested”), and “secondary TILs” are any TIL cell populations that have been expanded or proliferated as discussed herein, including, but not limited to bulk TILs, expanded TILs (“REP TILs”) as well as “reREP TILs” as discussed herein. reREP TILs can include for example second expansion TILs or second additional expansion TILs (such as, for example, those described in Step D of FIG. 8, including TILs referred to as reREP TILs).

TILs can generally be defined either biochemically, using cell surface markers, or functionally, by their ability to infiltrate tumors and effect treatment. TILs can be generally categorized by expressing one or more of the following biomarkers: CD4, CD8, TCR αβ, CD27, CD28, CD56, CCR7, CD45Ra, CD95, PD-1, and CD25. Additionally, and alternatively, TILs can be functionally defined by their ability to infiltrate solid tumors upon reintroduction into a patient. TILs may further be characterized by potency—for example, TILs may be considered potent if, for example, interferon (IFN) release is greater than about 50 pg/mL, greater than about 100 pg/mL, greater than about 150 pg/mL, or greater than about 200 pg/mL. TILs may be considered potent if, for example, interferon (IFNγ) release is greater than about 50 pg/mL, greater than about 100 pg/mL, greater than about 150 pg/mL, or greater than about 200 pg/mL, greater than about 300 pg/mL, greater than about 400 pg/mL, greater than about 500 pg/mL, greater than about 600 pg/mL, greater than about 700 pg/mL, greater than about 800 pg/mL, greater than about 900 pg/mL, greater than about 1000 pg/mL.

The term “deoxyribonucleotide” encompasses natural and synthetic, unmodified and modified deoxyribonucleotides. Modifications include changes to the sugar moiety, to the base moiety and/or to the linkages between deoxyribonucleotide in the oligonucleotide.

The term “RNA” defines a molecule comprising at least one ribonucleotide residue. The term “ribonucleotide” defines a nucleotide with a hydroxyl group at the 2′ position of a b-D-ribofuranose moiety. The term RNA includes double-stranded RNA, single-stranded RNA, isolated RNA such as partially purified RNA, essentially pure RNA, synthetic RNA, recombinantly produced RNA, as well as altered RNA that differs from naturally occurring RNA by the addition, deletion, substitution and/or alteration of one or more nucleotides. Nucleotides of the RNA molecules described herein may also comprise non-standard nucleotides, such as non-naturally occurring nucleotides or chemically synthesized nucleotides or deoxynucleotides. These altered RNAs can be referred to as analogs or analogs of naturally-occurring RNA.

The terms “pharmaceutically acceptable carrier” or “pharmaceutically acceptable excipient” are intended to include any and all solvents, dispersion media, coatings, antibacterial and antifungal agents, isotonic and absorption delaying agents, and inert ingredients. The use of such pharmaceutically acceptable carriers or pharmaceutically acceptable excipients for active pharmaceutical ingredients is well known in the art. Except insofar as any conventional pharmaceutically acceptable carrier or pharmaceutically acceptable excipient is incompatible with the active pharmaceutical ingredient, its use in therapeutic compositions of the invention is contemplated. Additional active pharmaceutical ingredients, such as other drugs, can also be incorporated into the described compositions and methods.

The terms “about” and “approximately” mean within a statistically meaningful range of a value. Such a range can be within an order of magnitude, preferably within 50%, more preferably within 20%, more preferably still within 10%, and even more preferably within 5% of a given value or range. The allowable variation encompassed by the terms “about” or “approximately” depends on the particular system under study, and can be readily appreciated by one of ordinary skill in the art. Moreover, as used herein, the terms “about” and “approximately” mean that dimensions, sizes, formulations, parameters, shapes and other quantities and characteristics are not and need not be exact, but may be approximate and/or larger or smaller, as desired, reflecting tolerances, conversion factors, rounding off, measurement error and the like, and other factors known to those of skill in the art. In general, a dimension, size, formulation, parameter, shape or other quantity or characteristic is “about” or “approximate” whether or not expressly stated to be such. It is noted that embodiments of very different sizes, shapes and dimensions may employ the described arrangements.

The transitional terms “comprising,” “consisting essentially of,” and “consisting of,” when used in the appended claims, in original and amended form, define the claim scope with respect to what unrecited additional claim elements or steps, if any, are excluded from the scope of the claim(s). The term “comprising” is intended to be inclusive or open-ended and does not exclude any additional, unrecited element, method, step or material. The term “consisting of” excludes any element, step or material other than those specified in the claim and, in the latter instance, impurities ordinary associated with the specified material(s). The term “consisting essentially of” limits the scope of a claim to the specified elements, steps or material(s) and those that do not materially affect the basic and novel characteristic(s) of the claimed invention. All compositions, methods, and kits described herein that embody the present invention can, in alternate embodiments, be more specifically defined by any of the transitional terms “comprising,” “consisting essentially of,” and “consisting of.”

The terms “antibody” and its plural form “antibodies” refer to whole immunoglobulins and any antigen-binding fragment (“antigen-binding portion”) or single chains thereof. An “antibody” further refers to a glycoprotein comprising at least two heavy (H) chains and two light (L) chains inter-connected by disulfide bonds, or an antigen-binding portion thereof. Each heavy chain is comprised of a heavy chain variable region (abbreviated herein as V_{H}) and a heavy chain constant region. The heavy chain constant region is comprised of three domains, CH1, CH2 and CH3. Each light chain is comprised of a light chain variable region (abbreviated herein as V_{L}) and a light chain constant region. The light chain constant region is comprised of one domain, C_{L}. The V_{H} and V_{L} regions of an antibody may be further subdivided into regions of hypervariability, which are referred to as complementarity determining regions (CDR) or hypervariable regions (HVR), and which can be interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen epitope or epitopes. The constant regions of the antibodies may mediate the binding of the immunoglobulin to host tissues or factors, including various cells of the immune system (e.g., effector cells) and the first component (Clq) of the classical complement system.

The term “antigen” refers to a substance that induces an immune response. In some embodiments, an antigen is a molecule capable of being bound by an antibody or a TCR if presented by major histocompatibility complex (MHC) molecules. The term “antigen”, as used herein, also encompasses T cell epitopes. An antigen is additionally capable of being recognized by the immune system. In some embodiments, an antigen is capable of inducing a humoral immune response or a cellular immune response leading to the activation of B lymphocytes and/or T lymphocytes. In some cases, this may require that the antigen contains or is linked to a Th cell epitope. An antigen can also have one or more epitopes (e.g., B- and T-epitopes). In some embodiments, an antigen will preferably react, typically in a highly specific and selective manner, with its corresponding antibody or TCR and not with the multitude of other antibodies or TCRs which may be induced by other antigens.

The terms “monoclonal antibody,” “mAb,” “monoclonal antibody composition,” or their plural forms refer to a preparation of antibody molecules of single molecular composition. A monoclonal antibody composition displays a single binding specificity and affinity for a particular epitope. Monoclonal antibodies specific to certain receptors can be made using knowledge and skill in the art of injecting test subjects with suitable antigen and then isolating hybridomas expressing antibodies having the desired sequence or functional characteristics. DNA encoding the monoclonal antibodies is readily isolated and sequenced using conventional procedures (e.g., by using oligonucleotide probes that are capable of binding specifically to genes encoding the heavy and light chains of the monoclonal antibodies). The hybridoma cells serve as a preferred source of such DNA. Once isolated, the DNA may be placed into expression vectors, which are then transfected into host cells such as E. coli cells, simian COS cells, Chinese hamster ovary (CHO) cells, or myeloma cells that do not otherwise produce immunoglobulin protein, to obtain the synthesis of monoclonal antibodies in the recombinant host cells. Recombinant production of antibodies will be described in more detail below.

The terms “antigen-binding portion” or “antigen-binding fragment” of an antibody (or simply “antibody portion” or “fragment”), as used herein, refers to one or more fragments of an antibody that retain the ability to specifically bind to an antigen. It has been shown that the antigen-binding function of an antibody can be performed by fragments of a full-length antibody. Examples of binding fragments encompassed within the term “antigen-binding portion” of an antibody include (i) a Fab fragment, a monovalent fragment consisting of the V_{L}, V_{H}, C_{L} and CH1 domains; (ii) a F(ab′)2 fragment, a bivalent fragment comprising two Fab fragments linked by a disulfide bridge at the hinge region; (iii) a Fd fragment consisting of the V_{H} and CH1 domains; (iv) a Fv fragment consisting of the V_{L} and V_{H} domains of a single arm of an antibody, (v) a domain antibody (dAb) fragment (Ward, et al., Nature, 1989, 341, 544-546), which may consist of a V_{H} or a V_{L} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules known as single chain Fv (scFv); see, e.g., Bird, et al., Science 1988, 242, 423-426; and Huston, et al., Proc. Natl. Acad. Sci. USA 1988, 85, 5879-5883). Such scFv antibodies are also intended to be encompassed within the terms “antigen-binding portion” or “antigen-binding fragment” of an antibody. These antibody fragments are obtained using conventional techniques known to those with skill in the art, and the fragments are screened for utility in the same manner as are intact antibodies. In some embodiments, a scFv protein domain comprises a V_{H} portion and a V_{L} portion. A scFv molecule is denoted as either V_{L}-L-V_{H} if the V_{L} domain is the N-terminal part of the scFv molecule, or as V_{H}-L-V_{L} if the V_{H} domain is the N-terminal part of the scFv molecule. Methods for making scFv molecules and designing suitable peptide linkers are described in U.S. Pat. Nos. 4,704,692, 4,946,778, R. Raag and M. Whitlow, “Single Chain Fvs.” FASEB Vol 9:73-80 (1995) and R. E. Bird and B. W. Walker, Single Chain Antibody Variable Regions, TIBTECH, Vol 9: 132-137 (1991), the disclosures of which are incorporated by reference herein.

The term “human antibody,” as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (e.g., mutations introduced by random or site-specific mutagenesis in vitro or by somatic mutation in vivo). The term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In some embodiments, the human monoclonal antibodies are produced by a hybridoma which includes a B cell obtained from a transgenic nonhuman animal, e.g., a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (such as a mouse) that is transgenic or transchromosomal for human immunoglobulin genes or a hybridoma prepared therefrom (described further below), (b) antibodies isolated from a host cell transformed to express the human antibody, e.g., from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

As used herein, “isotype” refers to the antibody class (e.g., IgM or IgG1) that is encoded by the heavy chain constant region genes.

The phrases “an antibody recognizing an antigen” and “an antibody specific for an antigen” are used interchangeably herein with the term “an antibody which binds specifically to an antigen.”

The term “human antibody derivatives” refers to any modified form of the human antibody, including a conjugate of the antibody and another active pharmaceutical ingredient or antibody. The terms “conjugate,” “antibody-drug conjugate”, “ADC,” or “immunoconjugate” refers to an antibody, or a fragment thereof, conjugated to another therapeutic moiety, which can be conjugated to antibodies described herein using methods available in the art.

The terms “humanized antibody,” “humanized antibodies,” and “humanized” are intended to refer to antibodies in which CDR sequences derived from the germline of another mammalian species, such as a mouse, have been grafted onto human framework sequences. Additional framework region modifications may be made within the human framework sequences. Humanized forms of non-human (for example, murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a 15 hypervariable region of a non-human species (donor antibody) such as mouse, rat, rabbit or nonhuman primate having the desired specificity, affinity, and capacity. In some instances, Fv framework region (FR) residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin. For further details, see Jones, et al., Nature 1986, 321, 522-525; Riechmann, et al., Nature 1988, 332, 323-329; and Presta, Curr. Op. Struct. Biol. 1992, 2, 593-596. The antibodies described herein may also be modified to employ any Fc variant which is known to impart an improvement (e.g., reduction) in effector function and/or FcR binding. The Fc variants may include, for example, any one of the amino acid substitutions disclosed in International Patent Application Publication Nos. WO 1988/07089 A1, WO 1996/14339 A1, WO 1998/05787 A1, WO 1998/23289 A1, WO 1999/51642 A1, WO 99/58572 A1, WO 2000/09560 A2, WO 2000/32767 A1, WO 2000/42072 A2, WO 2002/44215 A2, WO 2002/060919 A2, WO 2003/074569 A2, WO 2004/016750 A2, WO 2004/029207 A2, WO 2004/035752 A2, WO 2004/063351 A2, WO 2004/074455 A2, WO 2004/099249 A2, WO 2005/040217 A2, WO 2005/070963 A1, WO 2005/077981 A2, WO 2005/092925 A2, WO 2005/123780 A2, WO 2006/019447 A1, WO 2006/047350 A2, and WO 2006/085967 A2; and U.S. Pat. Nos. 5,648,260; 5,739,277; 5,834,250; 5,869,046; 6,096,871; 6,121,022; 6,194,551; 6,242,195; 6,277,375; 6,528,624; 6,538,124; 6,737,056; 6,821,505; 6,998,253; and 7,083,784; the disclosures of which are incorporated by reference herein.

The term “chimeric antibody” is intended to refer to antibodies in which the variable region sequences are derived from one species and the constant region sequences are derived from another species, such as an antibody in which the variable region sequences are derived from a mouse antibody and the constant region sequences are derived from a human antibody.

A “diabody” is a small antibody fragment with two antigen-binding sites. The fragments comprises a heavy chain variable domain (V_{H}) connected to a light chain variable domain (V_{L}) in the same polypeptide chain (V_{H}-V_{L} or V_{L}-V_{H}). By using a linker that is too short to allow pairing between the two domains on the same chain, the domains are forced to pair with the complementary domains of another chain and create two antigen-binding sites. Diabodies are described more fully in, e.g., European Patent No. EP 404,097, International Patent Publication No. WO 93/11161; and Bolliger, et al., Proc. Natl. Acad. Sci. USA 1993, 90, 6444-6448.

The term “glycosylation” refers to a modified derivative of an antibody. An aglycoslated antibody lacks glycosylation. Glycosylation can be altered to, for example, increase the affinity of the antibody for antigen. Such carbohydrate modifications can be accomplished by, for example, altering one or more sites of glycosylation within the antibody sequence. For example, one or more amino acid substitutions can be made that result in elimination of one or more variable region framework glycosylation sites to thereby eliminate glycosylation at that site. Aglycosylation may increase the affinity of the antibody for antigen, as described in U.S. Pat. Nos. 5,714,350 and 6,350,861. Additionally or alternatively, an antibody can be made that has an altered type of glycosylation, such as a hypofucosylated antibody having reduced amounts of fucosyl residues or an antibody having increased bisecting GlcNac structures. Such altered glycosylation patterns have been demonstrated to increase the ability of antibodies. Such carbohydrate modifications can be accomplished by, for example, expressing the antibody in a host cell with altered glycosylation machinery. Cells with altered glycosylation machinery have been described in the art and can be used as host cells in which to express recombinant antibodies of the invention to thereby produce an antibody with altered glycosylation. For example, the cell lines Ms704, Ms705, and Ms709 lack the fucosyltransferase gene, FUT8 (alpha (1,6) fucosyltransferase), such that antibodies expressed in the Ms704, Ms705, and Ms709 cell lines lack fucose on their carbohydrates. The Ms704, Ms705, and Ms709 FUT8−/− cell lines were created by the targeted disruption of the FUT8 gene in CHO/DG44 cells using two replacement vectors (see e.g. U.S. Patent Publication No. 2004/0110704 or Yamane-Ohnuki, et al., Biotechnol. Bioeng., 2004, 87, 614-622). As another example, European Patent No. EP 1,176,195 describes a cell line with a functionally disrupted FUT8 gene, which encodes a fucosyl transferase, such that antibodies expressed in such a cell line exhibit hypofucosylation by reducing or eliminating the alpha 1,6 bond-related enzyme, and also describes cell lines which have a low enzyme activity for adding fucose to the N-acetylglucosamine that binds to the Fc region of the antibody or does not have the enzyme activity, for example the rat myeloma cell line YB2/0 (ATCC CRL 1662). International Patent Publication WO 03/035835 describes a variant CHO cell line, Lec 13 cells, with reduced ability to attach fucose to Asn(297)-linked carbohydrates, also resulting in hypofucosylation of antibodies expressed in that host cell (see also Shields, et al., J. Biol. Chem. 2002, 277, 26733-26740. International Patent Publication WO 99/54342 describes cell lines engineered to express glycoprotein-modifying glycosyl transferases (e.g., beta(1,4)-N-acetylglucosaminyltransferase III (GnTIII)) such that antibodies expressed in the engineered cell lines exhibit increased bisecting GlcNac structures which results in increased ADCC activity of the antibodies (see also Umana, et al., Nat. Biotech. 1999, 17, 176-180). Alternatively, the fucose residues of the antibody may be cleaved off using a fucosidase enzyme. For example, the fucosidase alpha-L-fucosidase removes fucosyl residues from antibodies as described in Tarentino, et al., Biochem. 1975, 14, 5516-5523.

“Pegylation” refers to a modified antibody, or a fragment thereof, that typically is reacted with polyethylene glycol (PEG), such as a reactive ester or aldehyde derivative of PEG, under conditions in which one or more PEG groups become attached to the antibody or antibody fragment. Pegylation may, for example, increase the biological (e.g., serum) half life of the antibody. Preferably, the pegylation is carried out via an acylation reaction or an alkylation reaction with a reactive PEG molecule (or an analogous reactive water-soluble polymer). As used herein, the term “polyethylene glycol” is intended to encompass any of the forms of PEG that have been used to derivatize other proteins, such as mono (C₁-C₁₀)alkoxy- or aryloxy-polyethylene glycol or polyethylene glycol-maleimide. The antibody to be pegylated may be an aglycosylated antibody. Methods for pegylation are known in the art and can be applied to the antibodies of the invention, as described for example in European Patent Nos. EP 0154316 and EP 0401384 and U.S. Pat. No. 5,824,778, the disclosures of each of which are incorporated by reference herein.

The term “biosimilar” means a biological product, including a monoclonal antibody or protein, that is highly similar to a U.S. licensed reference biological product notwithstanding minor differences in clinically inactive components, and for which there are no clinically meaningful differences between the biological product and the reference product in terms of the safety, purity, and potency of the product. Furthermore, a similar biological or “biosimilar” medicine is a biological medicine that is similar to another biological medicine that has already been authorized for use by the European Medicines Agency. The term “biosimilar” is also used synonymously by other national and regional regulatory agencies. Biological products or biological medicines are medicines that are made by or derived from a biological source, such as a bacterium or yeast. They can consist of relatively small molecules such as human insulin or erythropoietin, or complex molecules such as monoclonal antibodies. For example, if the reference IL-2 protein is aldesleukin (PROLEUKIN), a protein approved by drug regulatory authorities with reference to aldesleukin is a “biosimilar to” aldesleukin or is a “biosimilar thereof” of aldesleukin. In Europe, a similar biological or “biosimilar” medicine is a biological medicine that is similar to another biological medicine that has already been authorized for use by the European Medicines Agency (EMA). The relevant legal basis for similar biological applications in Europe is Article 6 of Regulation (EC) No 726/2004 and Article 10(4) of Directive 2001/83/EC, as amended and therefore in Europe, the biosimilar may be authorized, approved for authorization or subject of an application for authorization under Article 6 of Regulation (EC) No 726/2004 and Article 10(4) of Directive 2001/83/EC. The already authorized original biological medicinal product may be referred to as a “reference medicinal product” in Europe. Some of the requirements for a product to be considered a biosimilar are outlined in the CHMP Guideline on Similar Biological Medicinal Products. In addition, product specific guidelines, including guidelines relating to monoclonal antibody biosimilars, are provided on a product-by-product basis by the EMA and published on its website. A biosimilar as described herein may be similar to the reference medicinal product by way of quality characteristics, biological activity, mechanism of action, safety profiles and/or efficacy. In addition, the biosimilar may be used or be intended for use to treat the same conditions as the reference medicinal product. Thus, a biosimilar as described herein may be deemed to have similar or highly similar quality characteristics to a reference medicinal product. Alternatively, or in addition, a biosimilar as described herein may be deemed to have similar or highly similar biological activity to a reference medicinal product. Alternatively, or in addition, a biosimilar as described herein may be deemed to have a similar or highly similar safety profile to a reference medicinal product. Alternatively, or in addition, a biosimilar as described herein may be deemed to have similar or highly similar efficacy to a reference medicinal product. As described herein, a biosimilar in Europe is compared to a reference medicinal product which has been authorized by the EMA. However, in some instances, the biosimilar may be compared to a biological medicinal product which has been authorized outside the European Economic Area (a non-EEA authorized “comparator”) in certain studies. Such studies include for example certain clinical and in vivo non-clinical studies. As used herein, the term “biosimilar” also relates to a biological medicinal product which has been or may be compared to a non-EEA authorized comparator. Certain biosimilars are proteins such as antibodies, antibody fragments (for example, antigen binding portions) and fusion proteins. A protein biosimilar may have an amino acid sequence that has minor modifications in the amino acid structure (including for example deletions, additions, and/or substitutions of amino acids) which do not significantly affect the function of the polypeptide. The biosimilar may comprise an amino acid sequence having a sequence identity of 97% or greater to the amino acid sequence of its reference medicinal product, e.g., 97%, 98%, 99% or 100%. The biosimilar may comprise one or more post-translational modifications, for example, although not limited to, glycosylation, oxidation, deamidation, and/or truncation which is/are different to the post-translational modifications of the reference medicinal product, provided that the differences do not result in a change in safety and/or efficacy of the medicinal product. The biosimilar may have an identical or different glycosylation pattern to the reference medicinal product. Particularly, although not exclusively, the biosimilar may have a different glycosylation pattern if the differences address or are intended to address safety concerns associated with the reference medicinal product. Additionally, the biosimilar may deviate from the reference medicinal product in for example its strength, pharmaceutical form, formulation, excipients and/or presentation, providing safety and efficacy of the medicinal product is not compromised. The biosimilar may comprise differences in for example pharmacokinetic (PK) and/or pharmacodynamic (PD) profiles as compared to the reference medicinal product but is still deemed sufficiently similar to the reference medicinal product as to be authorized or considered suitable for authorization. In certain circumstances, the biosimilar exhibits different binding characteristics as compared to the reference medicinal product, wherein the different binding characteristics are considered by a Regulatory Authority such as the EMA not to be a barrier for authorization as a similar biological product. The term “biosimilar” is also used synonymously by other national and regional regulatory agencies.

### III. Immunomodulatory Agent Associated Tumor Infiltrating Lymphocytes

Provided herein are modified tumor infiltrating lymphocytes (TIL) that include one or more immunomodulatory agents associated with the TIL cell surface. In some embodiments, the subject modified TILs exhibit enhanced in vivo survival, proliferation and/or anti-tumor effects in a patient recipient.

The immunomodulatory agent can be attached to the TIL disclosed herein (e.g. therapeutics TILs provided herein) using any suitable method. In some embodiments the one or more immunomodulatory agents are part of an immunomodulatory fusion protein that is attached to the TIL cell surface. In some embodiments, the one or more immunomodulatory agents are included as part of nanoparticles that are associated with the TIL cell surfaces. The immunomodulatory agents can be any immunomodulatory agent that promotes TIL survival proliferation, and/or anti-tumor effects in a patient recipient. In some embodiments, the immunomodulatory agent is a cytokine (e.g., an interleukin). In exemplary embodiments, the TILs include IL-12, IL-15, and/or IL-21.

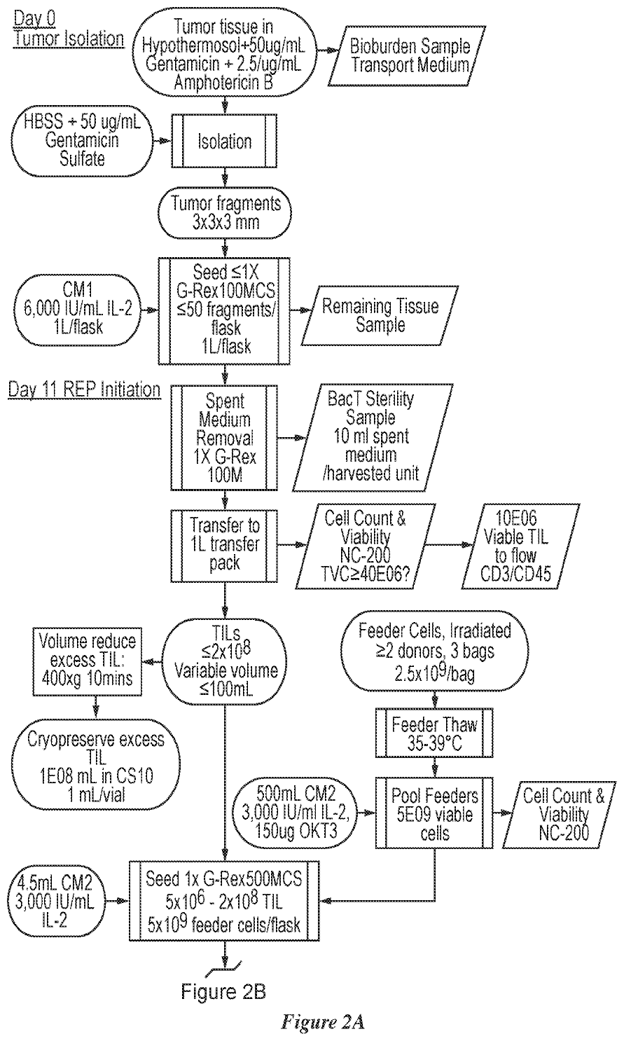

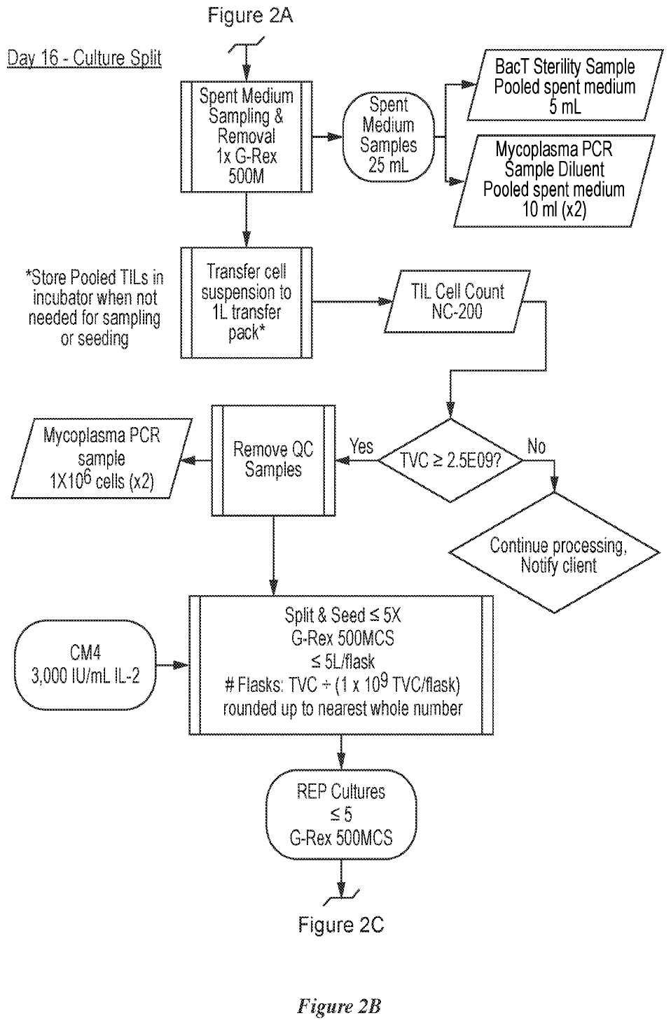

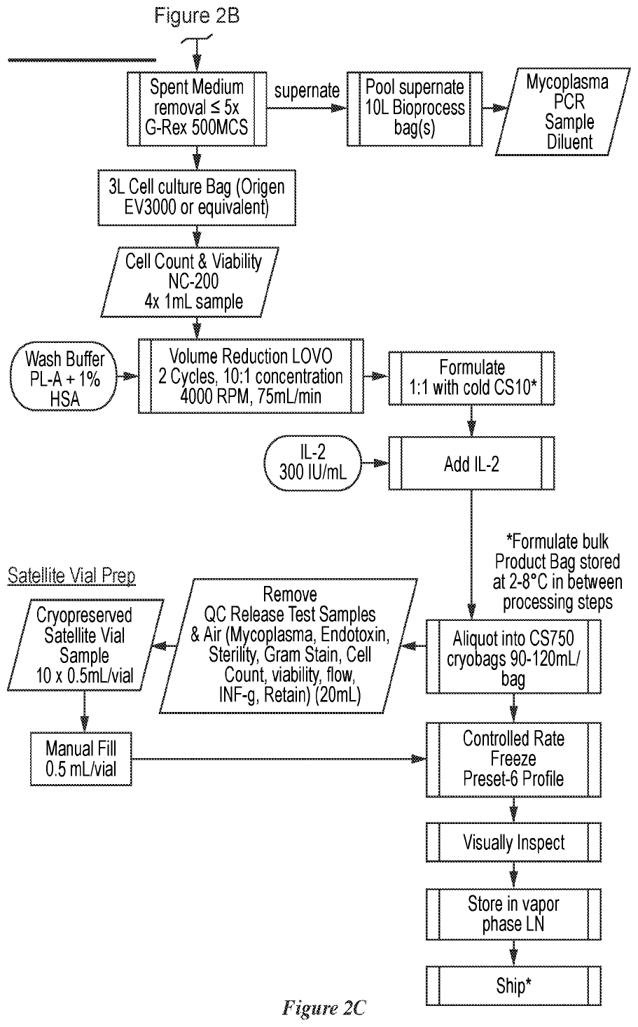

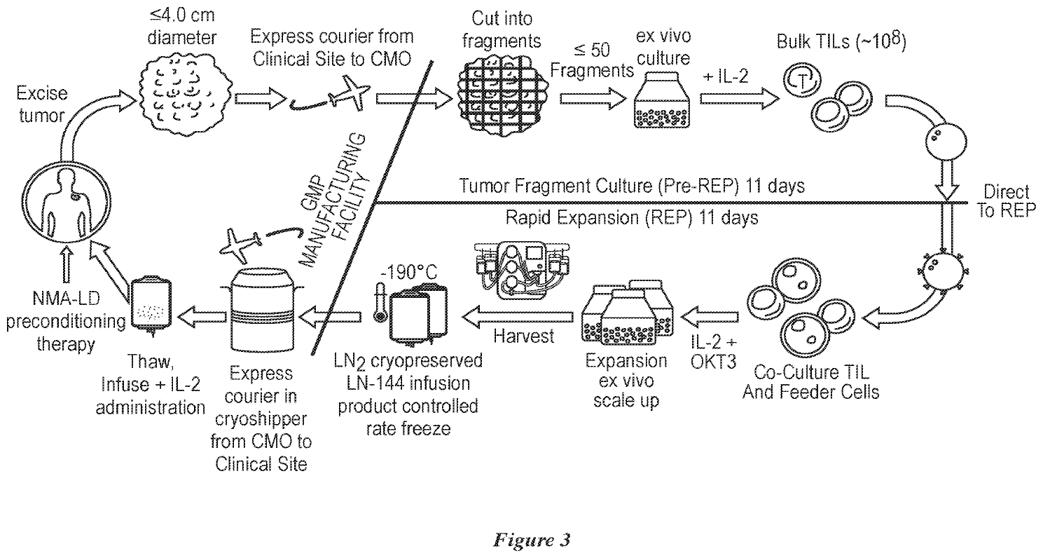

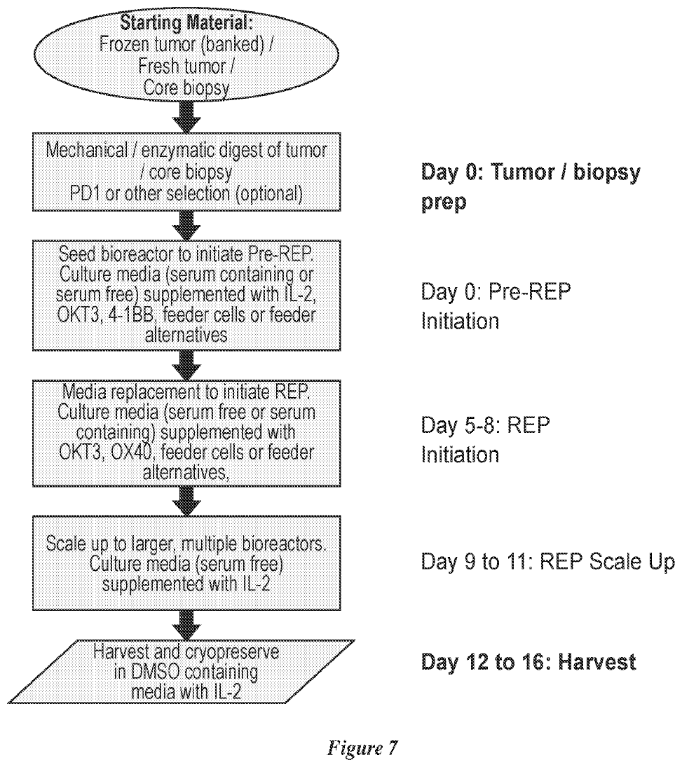

Any suitable TIL population can be modified to produce the subject compositions, including TILs produced using the manufacturing processes described herein. In some embodiments, the modified TILs are derived from TILs produced during any of the steps of the Process 2A method disclosure herein (see, e.g., FIGS. 2-6). In exemplary embodiments, the modified TILs are derived from TILs produced during any of the steps of the GEN 3 method disclosure herein (see, e.g., FIG. 7). In some embodiments, the TILs are PD-1 positive TILs that are derived from the methods disclosed herein.

In some embodiments, the TILs are further modified by a gene-editing process as disclosed herein, for example, CRISPR methods, TALE methods, ZFN methods, tCas-CLOVER methods, shRNA methods, or a combination thereof, to alter the expression of one or more immune checkpoint genes in the TIL population. Non-limiting examples of immune checkpoint genes that may be silenced or inhibited by gene-editing methods of the present invention include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, BAFF (BR3), CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, and BCOR.

Aspects of the subject modified TILs are further detailed herein.

### A. Immunomodulatory Fusion Proteins

In some embodiments, the modified TILs provided herein includes an immunomodulatory fusion protein that includes an immunomodulatory agent (e.g., a cytokine) linked to a moiety that facilitates the tethering of the immunomodulatory agent to surface of the TILs. In some embodiments, the fusion protein includes a cell membrane anchor moiety (a transmembrane domain). In certain embodiments, the fusion protein includes a TIL surface antigen binding moiety that binds to a TIL surface antigen. Aspects of these fusion proteins are further discussed in detail below.

Any suitable genetic modification method can be used to genetically modify the subject TILs to include such immunomodulatory fusion proteins, including, for example, any of the gene editing methods described herein. In some embodiments, the genetic modification method is a CRISPR, TALE, zinc finger, or Cas-CLOVER method described herein. In some embodiments, such modified TILs are produced using any one of the retroviral methods (e.g., lentiviral methods) provided herein. In some embodiments, such modified TILs are produced using any of the transposon/transposase systems described herein, e.g., a piggyBac method (e.g., piggyBac transposons and transposases or piggyBac-like transposons and transposases), a Sleeping Beauty method (e.g., Sleeping Beauty or Sleeping Beauty-like transposons and transposases), a Helraiser method (e.g., Helraiser ahd Helraiser-like transposons and transposases), and a Tol2 method (e.g., Tol2 and Tol2-like transposons and transposases).

### 1. Membrane Anchored Immunomodulatory Fusion Proteins

In some embodiments, the modified TILs provided herein include a membrane anchored immunomodulatory fusion protein. The membrane anchored immunomodulatory fusion protein includes one or more of the immunomodulatory agents (e.g., a cytokine) linked to a cell membrane anchor moiety. In such embodiments, the membrane anchored immunomodulatory agent is tethered to the TIL surface membrane via the cell membrane anchor moiety, thus allowing the immunomodulatory agent to exert its effects in a targeted manner.

The immunomodulatory agent can be any suitable immunomodulatory agent including, for example, any of the immunomodulatory agents provided herein. In some embodiments, the immunomodulatory agent is an interleukin that promotes an anti-tumor response. In some embodiments, the immunomodulatory agent is a cytokine. In particular embodiments, the immunomodulatory agent is IL-2, IL-12, IL-15, IL-18, IL-21, or a CD40 agonist (e.g., CD40L or agonistic anti-CD40 binding domain (e.g., an anti-CD40 scFv)) or a bioactive variant thereof. In certain embodiments, two or more different a membrane anchored immunomodulatory fusion proteins are expressed on a TIL surface. In exemplary embodiments, a TIL includes 2, 3, 4, 5, 6, 7, 8, 9 or 10 different membrane anchored immunomodulatory fusion proteins.

The immunomodulatory agent is linked to a cell membrane anchor moiety that allows the tethering of the immunomodulatory agent to the TIL cell surface. Suitable cell membrane anchor moieties include, for example, transmembrane domains of endogenous TIL cell surface proteins and fragments thereof. Exemplary transmembrane domains that can be used in the subject fusion proteins, include for example, B7-1, B7-2, and CD8a transmembrane domains and fragments thereof. In some embodiments, the cell membrane anchor moiety further includes a transmembrane and intracellular domain of an endogenous TIL cell surface protein or fragment thereof. In some embodiments, the cell membrane anchor moiety is a B7-1, B7-2 or CD8a transmembrane-intracellular domain or fragment thereof. In certain embodiments, the cell membrane anchor moiety is a CD8a transmembrane domain having the amino acid sequence of IYIWAPLAGTCGVLLLSLVIT (SEQ ID NO:238). In certain embodiments, the cell membrane anchor moiety is a B7-1 transmembrane-intracellular domain having the amino acid sequence of LLPSWAITLISVNGIFVICCLTYCFAPRCRERRRNERLRRESVRPV (SEQ ID NO:239).

In certain embodiments, the cell membrane anchor moiety is a non-peptide cell membrane anchor moiety. In exemplary embodiments, the non-peptide cell membrane anchor moiety is a glycophosphatidylinositol (GPI) anchor. GPI anchors have a structure that includes a phosphoethanolamine linker, glycan core, and phospholipid tail. In some embodiments, the glycan core is modified with one or more side chains. In some embodiments, the glycan core is modified with one or more of the following side chains: a phosphoethanolamine group, mannose, galactose, sialic acid, or other sugars.

The membrane anchored immunomodulatory fusion protein include linkers that allow for the linkage of components of the membrane anchored immunomodulatory fusion protein (e.g. an immunomodulatory agent to a cell membrane anchor moiety). Suitable linkers include linkers that are at least about 5, 6, 7, 8, 9, 10, 11, 12, 13, 14, 15, 16, 17, 18, 19, 20, 21, 22, 23, 24, 25, 26, 27, 28, 29 or 30 amino acid residues in length. In some embodiments, the linker is 5-10, 10-15, 15-20, 20-25, 25-30, 30-35, 35-40, 45-50, 50-60 amino acids in length. Suitable linkers include, but are not limited: a cleavable linker, a non-cleavable linker, a peptide linker, a flexible linker, a rigid linker, a helical linker, or a non-helical linker. In some embodiments, the linker is a peptide linker that optionally comprises Gly and Ser. In certain embodiments, the peptide linker utilize a glycine-serine polymer, including for example (GS)n (SEQ ID NO:240), (GSGGS)n (SEQ ID NO:241), (GGGS)n (SEQ ID NO:242), (GGGGS)n (SEQ ID NO:243), (GGGGGS)n (SEQ ID NO:244), and (GGGGGGS)n (SEQ ID NO:245), where n is an integer of at least one (and generally from 3 to 10). Additional linkers that can be used with the present compositions and methods are described in U.S. Patent Publication Nos. US 2006/0074008, US 20050238649, and US 2006/0024317, each of which is incorporated by reference herein in its entirety, and particularly in pertinent parts related to linkers. In some embodiments, the peptide linker is SGGGGSGGGGSGGGGSGGGGSGGGSLQ (SEQ ID NO:246).

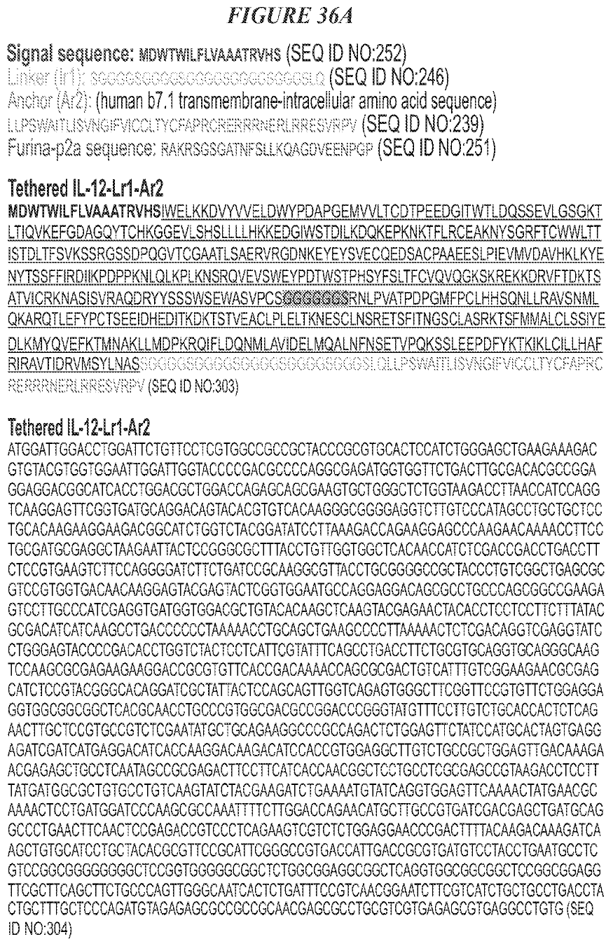

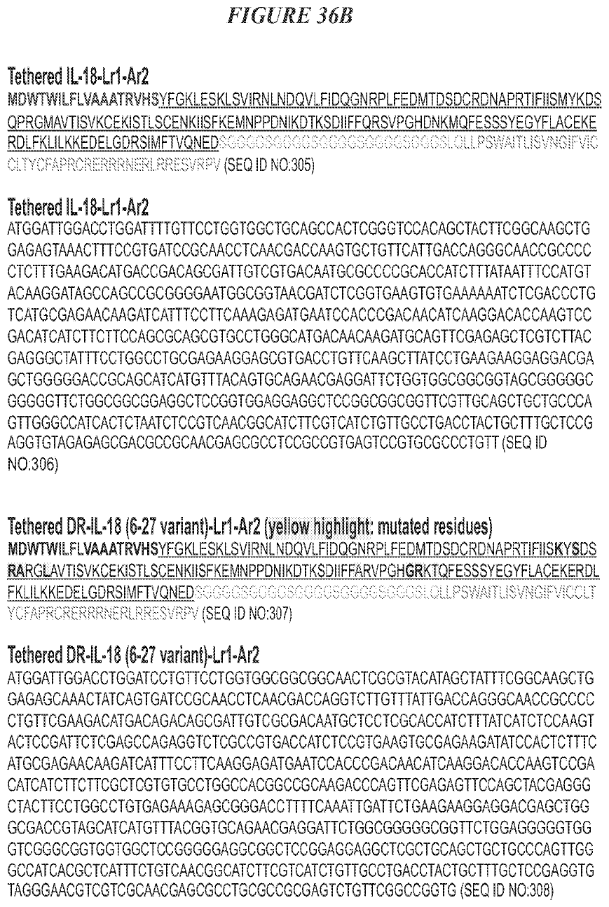

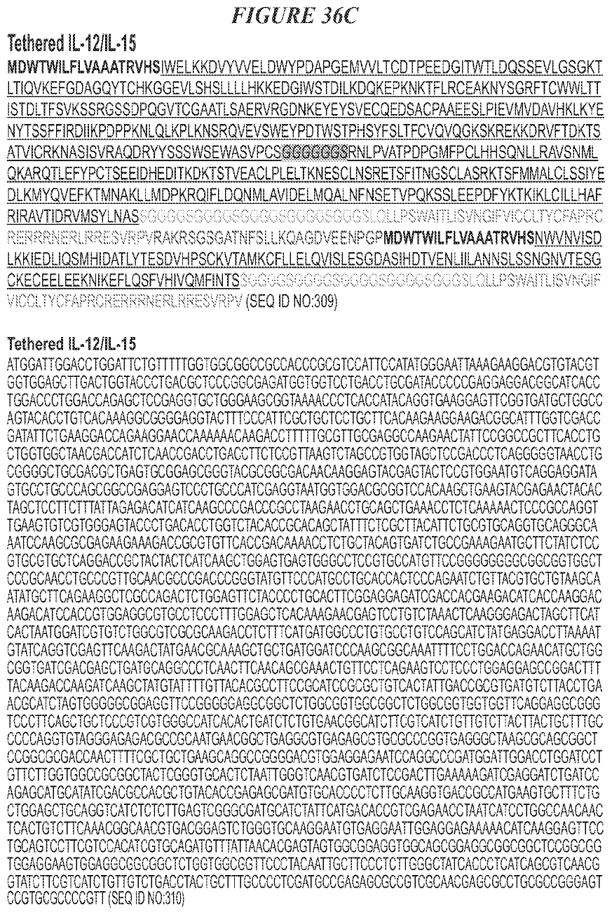

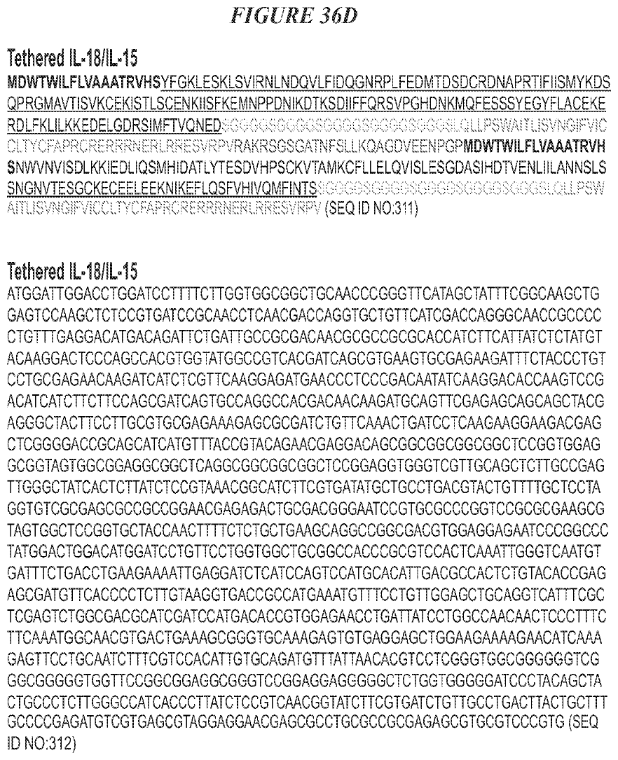

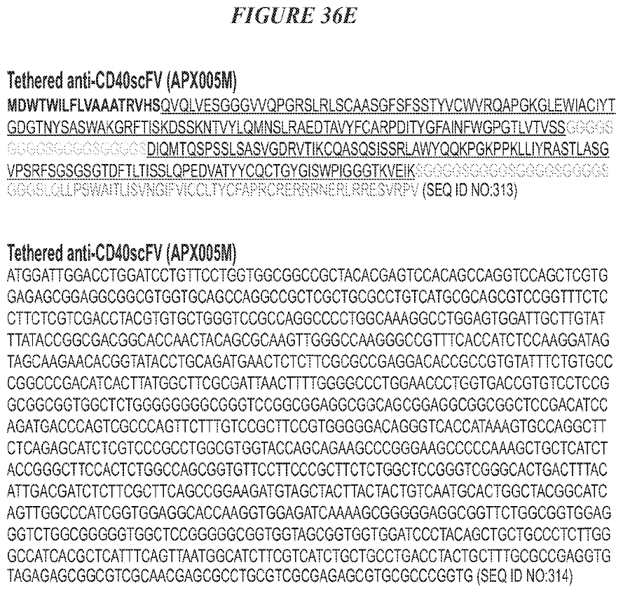

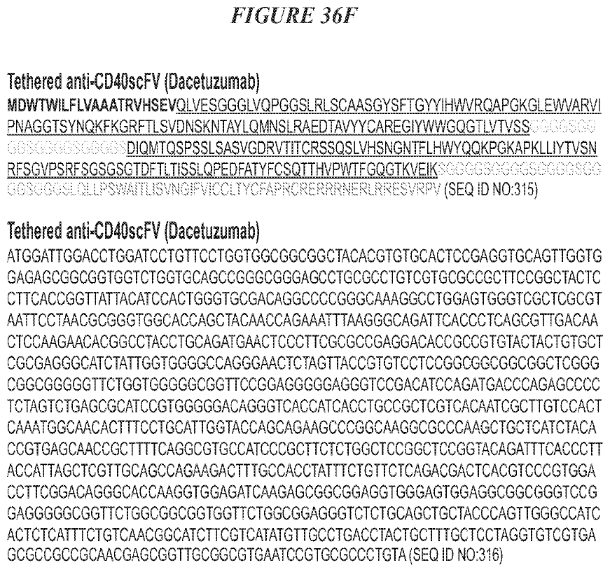

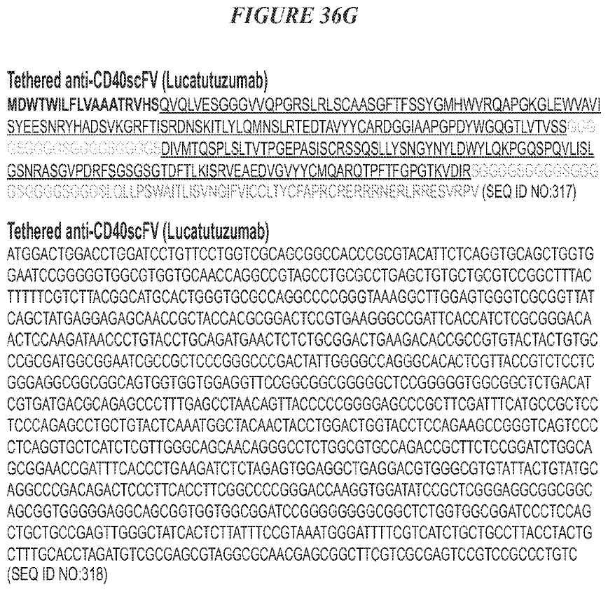

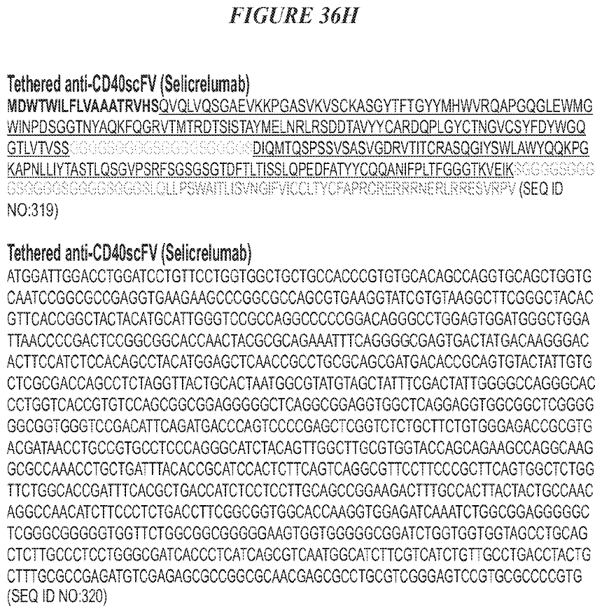

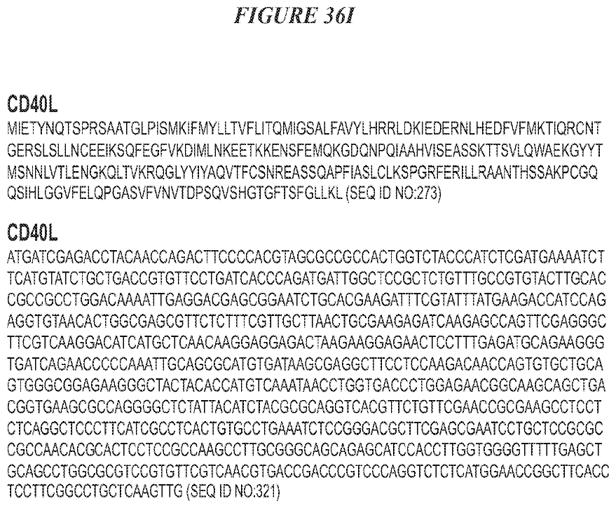

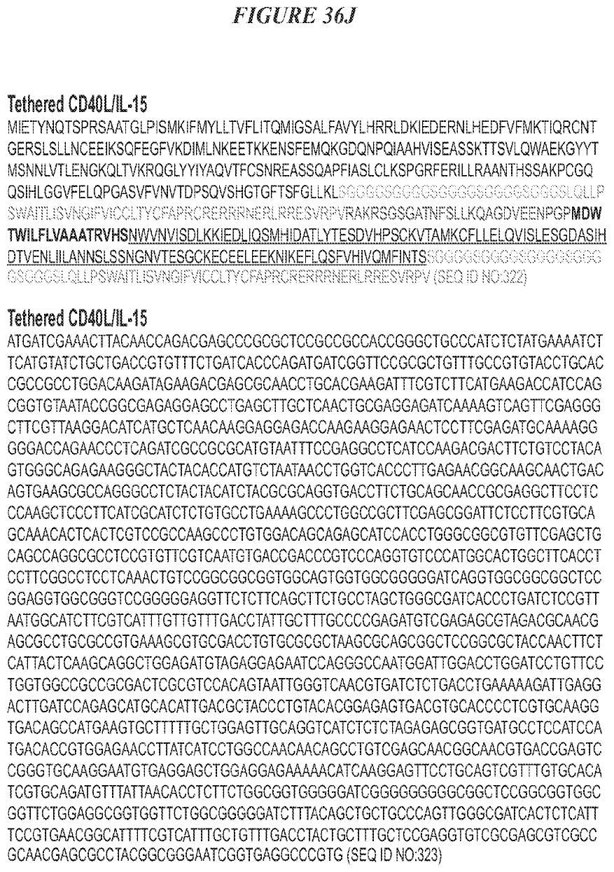

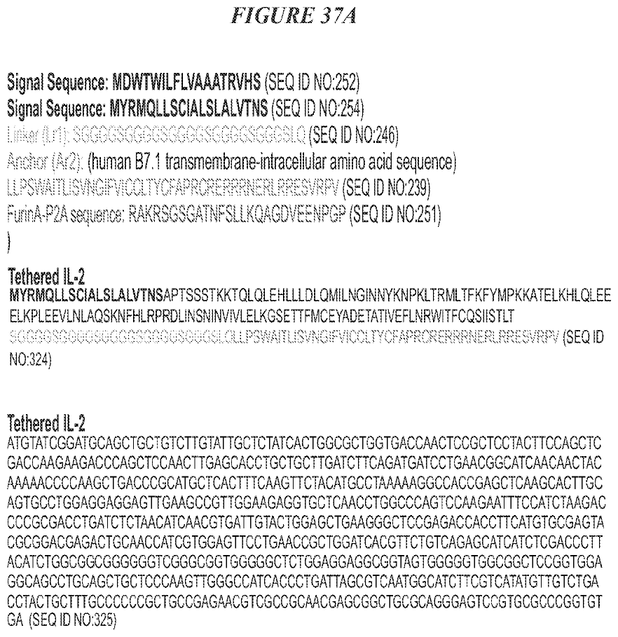

In some embodiments, the linker is a cleavable linker. In exemplary embodiments, the cleavable linker allows for the release of the immunomodulatory agent into the tumor microenvironment. Cleavable linkers are also useful in embodiments, wherein two membrane anchored immunomodulatory fusion proteins are co-expressed in the same TIL (see, e.g., FIG. 36 and Tables 58 and 59). In exemplary embodiments, the linker is a self-cleaving 2A peptide. See, e.g., Liu et al., Sci. Rep. 7(1):2193 (2017), which is incorporated by reference in relevant parts relating to 2A peptides. 2A peptides are viral oligopeptides that mediate cleavage of polypeptides during translation in eukaryotic cells. In some embodiments, the 2A peptide includes a C-terminus having the amino acid sequence GDVEXiNPGP (SEQ ID NO:247), wherein Xi is any naturally occurring amino acid residue. In certain embodiments, the 2A peptide is a porcine teschovirus-1 2A peptide (GSGATNFSLLKQAGDVEENPGP, SEQ ID NO:248). In some embodiments, the 2A peptide is an equine rhinitis A virus 2A peptide (GSGQCTNYALLKLAGDVESNPGP, SEQ ID NO:249). In certain embodiments, the 2A peptide is a foot-and-mouth disease virus 2A peptide: (GSGEGRGSLLTCGDVEENPGP, SEQ ID NO:250). In some embodiments, the cleavable linker includes a furin-cleavable sequence. Exemplary furin-cleavable sequences are described for example, Duckert et al., Protein Engineering, Design & Selection 17(1):107-112 (2004), and U.S. Pat. No. 8,871,906, each of which is incorporated herein by reference, particularly in relevant parts relating to furin-cleavable sequences. In some embodiments, the linker includes a 2A peptide and a furin-cleavable sequence. In exemplary embodiments, the furin-cleavable 2A peptide includes the amino acid sequence RAKRSGSGATNFSLLKQAGDVEENPGP (SEQ ID NO:251).

In some embodiments, the immunomodulatory agents are attached in the membrane anchored immunomodulatory fusion protein by a degradable linker (e.g., a disulfide linker) such that under physiological conditions, the linker degrades, thereby releasing the immunomodulatory agent. In some embodiments, the immunomodulatory agents are reversibly linked to functional groups through a degradable linker such that under physiological conditions, the linker degrades and releases the immunomodulatory agent. Suitable degradable linkers include, but are not limited to: a protease sensitive linker that is sensitive to one or more enzymes present in biological media such as proteases in a tumor microenvironment such a matrix metalloproteases present in a tumor microenvironment or in inflamed tissue (e.g. matrix metalloproteinase 2 (MMP2) or matrix metalloproteinase 9 (MMP9)).

In other embodiments, the components of the membrane anchored immunomodulatory fusion protein are linked by an enzyme-sensitive linker. Exemplary cleavable linker include those that are recognized by one of the following enzymes: metalloprotease MMP-1, MMP-2, MMP-3, MMP-8, MMP-9, MMP-14, plasmin, PSA, PSMA, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. See, e.g., U.S. Pat. Nos. 8,541,203 and 8,580,244, each of which is incorporated by reference in its entirety and in pertinent parts related to cleavable linkers.

In certain embodiments, the membrane anchored immunomodulatory fusion protein includes a signal peptide that facilitates the translocation of the fusion protein to the TIL cell membrane. Any suitable signal peptide that facilities the localization of the fusion protein to the TIL cell membrane can be used. In some embodiments, the signal peptide does not interfere with the bioactivity of the immunomodulatory agent. Exemplary signal peptide sequences include, but are not limited to: human granulocyte-macrophage colony-stimulating factor (GM-CSF) receptor signal sequence, human prolactin signal sequence, and human IgE signal sequence. In certain embodiments, the fusion protein includes a human IgE signal sequence. In exemplary embodiments, the human IgE signal sequence has the amino acid sequence MDWTWILFLVAAATRVHS (SEQ ID NO:252). In some embodiments, the human IgE signal sequence includes the amino acid sequence NIKGSPWKGSLLLLLVSNLLLCQSVAP (SEQ ID NO:253). In some embodiments, the signal peptide sequence is an IL-2 signal sequence having the amino acid sequence MYRMQLLSCIALSLALVTNS (SEQ ID NO:254).

In some embodiments, the membrane anchored immunomodulatory fusion protein is according to the formula, from N- to C-terminus:

S-IA-L-C,

wherein S is a signal peptide, IA is an immunomodulatory agent, L is a linker and C is a cell membrane anchor moiety.

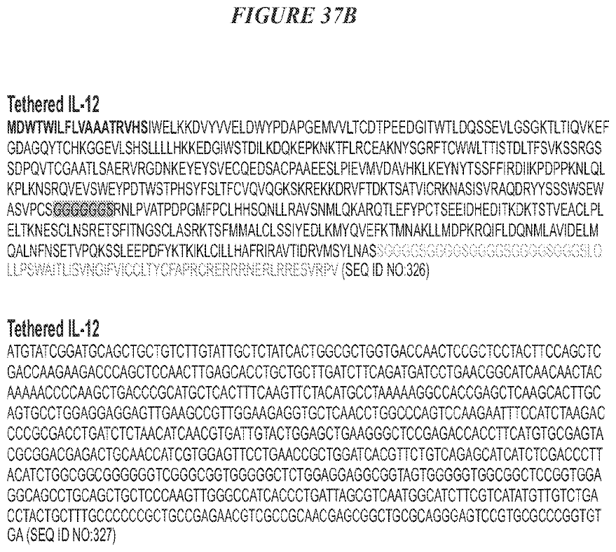

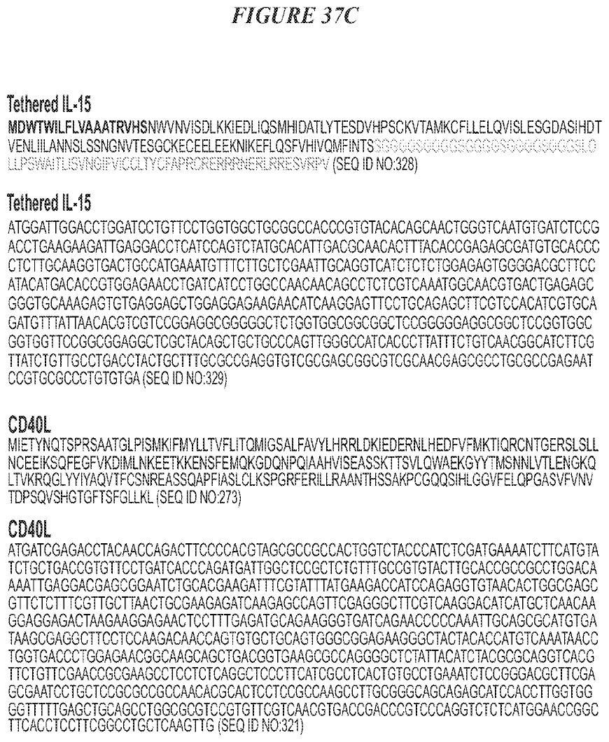

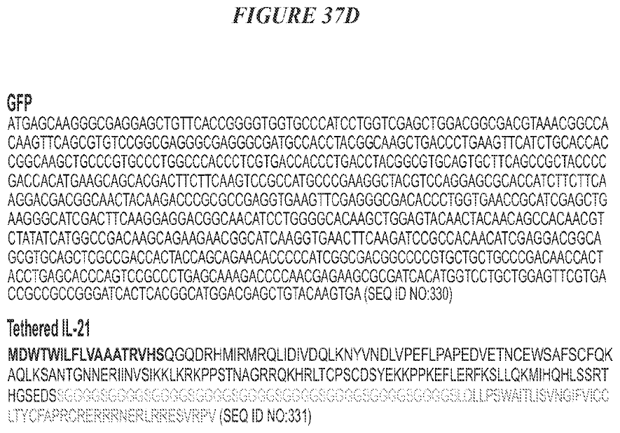

In some embodiments, the signal peptide S is any one of SEQ ID NOs:252-254. In some embodiments, the cell membrane anchor moiety is SEQ ID NO:277. In exemplary embodiments, the immunomodulatory agent is IL-2, IL-12, IL-15, IL-18, IL-21, or a CD40 agonist (e.g., CD40L or an anti-CD40 scFv as described herein). In some embodiments, C is a B7-1 transmembrane-intracellular domain (e.g., SEQ ID NO:239). Exemplary membrane anchored immunomodulatory fusion proteins according to the above formula are depicted in FIGS. 36 and 37.

In some embodiments, the TIL includes two or more different membrane anchored immunomodulatory fusion proteins according to the formula, from N- to C-terminus: S-IA-L-C, wherein each of the different membrane anchored immunomodulatory fusion proteins includes a different immunomodulatory agent. In some embodiments, the two or more different immunomodulatory agents are selected from the group consisting of: IL-12 and IL-15, IL-15 and IL-18, CD40L and IL-15, IL-15 and IL-21, and IL-2 and IL-12.

In some embodiments that includes two membrane anchored immunomodulatory fusion proteins, the membrane anchored immunomodulatory fusion proteins are arranged according to the formula, from N- to C-terminus:

S1-IA1-L1-C1-L2-S2-IA2-L3-C2,

wherein S1 and S2 are each a signal peptide, IA1 and IA2 are each an immunomodulatory agent, L1-L3 are each a linker, and C1 and C2 are each a cell membrane anchor moiety. In some embodiments, IA1 and IA2 are the same immunomodulatory agent. In certain embodiments, IA1 and IA2 are different immunomodulatory agents. Suitable immunomodulatory agents including any of those described herein. In some embodiments, IA1 and IA2 are independently selected from IL-2, IL-12, IL-15, IL-18, IL-21, a CD40 agonist (e.g., CD40L or an agonistic anti-CD40 binding domain (e.g., an anti-CD40 scFv)) or a bioactive variant thereof. In some embodiments, IA1 and IA2 are selected from the group consisting of: IL-12 and IL-15, IL-15 and IL-18, CD40L and IL-15, IL-15 and IL-21, and IL-2 and IL-12. In some embodiments, one or more of L1-L3 is a cleavable linker. In some embodiments two or more of L1-L3 are different linkers. In exemplary embodiments L2 is a cleavable linker. In some embodiments, L2 is furin cleavable P2A linker (e.g., SEQ ID NO:251). In some embodiments, C1 and C2 are independently transmembrane domains and/or transmembrane-intracellular domains. In certain embodiments C1 and C2 are the same. In exemplary embodiments, C1 and C2 are each a B7-1 transmembrane-intracellular domain (e.g., SEQ ID NO:239). In exemplary embodiments, C1 and C2 are different. Exemplary constructs that include two membrane anchored immunomodulatory fusion proteins according to the above formula are depicted in FIG. 36, and Tables 58 and 59.

Modified TILs that include cell membrane anchored immunomodulatory fusion proteins associated with their surfaces can be made by genetically modifying a populations of TILs to include a nucleic acid encoding the fusion protein. Any suitable genetic modification method can be used to produce such modified TILs including, for example, CRISPR, TALE, zinc finger, and Cas-CLOVER method described herein.

Any suitable population of TILs can be genetically modified to make the subject modified TIL compositions. In some embodiments, a population TILs produced during any of the steps of the Process 2A method disclosure herein (see, e.g., FIGS. 2-6) is genetically modified to produce the subject modified TILs. In exemplary embodiments, a population TILs produced during any of the steps of the GEN 3 method disclosure herein (see, e.g., FIG. 7) is genetically modified to produce the subject modified TILs. In exemplary embodiments, TILs produced from the second step in the Process 2A method and/or the rapid expansion step in the GEN 3 method provided herein are genetically modified to produce the subject modified TILs. In some embodiments, PD-1 positive TILs that have been preselected using the methods described herein are genetically modified to produce the subject modified TILs.

Any suitable population of TILs can be transiently modified to make the subject transiently modified TIL compositions. In some embodiments, a population of TILs produced during any of the steps of the Process 2A method disclosure herein (see, e.g., FIGS. 2-6) is transfected with nucleic acid encoding a cell membrane anchored immunomodulatory fusion protein to transiently express the cell membrane anchored immunomodulatory fusion protein in the subject transiently modified TILs. In exemplary embodiments, a population of TILs produced during any of the steps of the GEN 3 method disclosure herein (see, e.g., FIG. 7) is transfected with nucleic acid encoding a cell membrane anchored immunomodulatory fusion protein to transiently express the cell membrane anchored immunomodulatory fusion protein in the subject transiently modified TILs. In exemplary embodiments, TILs produced from the first expansion step in the Process 2A method and/or the priming expansion step in the GEN 3 method provided herein are transfected with nucleic acid encoding a cell membrane anchored immunomodulatory fusion protein to transiently express the cell membrane anchored immunomodulatory fusion protein in the subject transiently modified TILs. In exemplary embodiments, TILs produced from the second expansion step in the Process 2A method and/or the rapid expansion step in the GEN 3 method provided herein are transfected with nucleic acid encoding a cell membrane anchored immunomodulatory fusion protein to transiently express the cell membrane anchored immunomodulatory fusion protein in the subject transiently modified TILs. In some embodiments, PD-1 positive TILs that have been preselected using the methods described herein are transfected with nucleic acid encoding a cell membrane anchored immunomodulatory fusion protein to transiently express the cell membrane anchored immunomodulatory fusion protein in the subject transiently modified TILs.

Also provided herein are nucleic acids encoding the membrane anchored immunomodulatory fusion proteins, expression vectors that include such nucleic acids, and host cells that include the nucleic acids or expression vectors. Any suitable promoter can be used for the expression of the membrane anchored immunomodulatory fusion protein. In exemplary embodiments, the promoter is an inducible promoter. Vectors for expression of the subject membrane anchored immunomodulatory fusion proteins include, but are not limited to adenoviral vectors, retroviral vectors, lentiviral vectors, and adeno-associated vectors (AAV). In some embodiments, a piggyBac transposon is used for expression of the subject membrane anchored immunomodulatory fusion proteins. Exemplary nucleic acids that encode for exemplary membrane anchored immunomodulatory fusion proteins and components of such fusion proteins are depicted in FIGS. 36 and 37, and Tables 58 and 59.

In some embodiments, the nucleic acids encoding the membrane anchored immunomodulatory fusion protein is mRNA. In exemplary embodiments, the mRNA includes one or more modifications that improves intracellular stability and/or translation efficiency of the mRNA. In some embodiments, the mRNA includes a 5′ cap or cap analog that improves mRNA half-life. Exemplary cap structures, include, but are not limited to ARCA, mCAP, m⁷GpppN (cap 0), m⁷GpppNm (cap 1), and m⁷GpppNmpNm (cap 2) caps. In some embodiments, the 5′ cap is according to the formula: ^{m7}Gppp[N_{2Ome}]ₙ[N]ₘ wherein ^{m7}G is N7-methylated guanosine or any guanosine analog, N is any natural, modified or unnatural nucleoside, “n” can be any integer from 0 to 4 and “m” can be an integer from 1 to 9. Exemplary 5′ caps are disclosed in U.S. Pat. No. 10,703,789 and WO2017053297, which are incorporated by reference in their entirety, and specifically for disclosures relating to 5′ caps and cap analogs.

In some embodiments, the nucleic acids encoding the membrane anchored immunomodulatory fusion protein is mRNA further includes a 3′ untranslated region (UTR) or modified UTR. 3′ UTRs are known to have stretches of adenosines and uridines. These AU rich signatures are particularly prevalent in genes with high rates of turnover. Based on their sequence features and functional properties, the AU rich elements (AREs) can be separated into three classes (Chen et al, 1995): Class I AREs contain several dispersed copies of an AUUUA motif within U-rich regions. C-Myc and MyoD contain class I AREs. Class II AREs possess two or more overlapping UUAUUUA(U/A)(U/A) nonamers. Molecules containing this type of AREs include GM-CSF and TNF-a. Class III ARES are less well defined. These U rich regions do not contain an AUUUA motif c-Jun and Myogenin are two well-studied examples of this class. Most proteins binding to the AREs are known to destabilize the messenger, whereas members of the ELAV family, most notably HuR, have been documented to increase the stability of mRNA. HuR binds to AREs of all the three classes. Engineering the HuR specific binding sites into the 3′ UTR of nucleic acid molecules will lead to HuR binding and thus, stabilization of the message in vivo.

Introduction, removal or modification of 3′ UTR AU rich elements (AREs) can be used to modulate the stability of the nucleic acids described herein. When engineering specific nucleic acids, one or more copies of an ARE can be introduced to make polynucleotides of the invention less stable and thereby curtail translation and decrease production of the resultant protein. Likewise, AREs can be identified and removed or mutated to increase the intracellular stability and thus increase translation and production of the resultant protein. Transfection experiments can be conducted in relevant cell lines, using nucleic acids, and protein production can be assayed at various time points post-transfection. For example, cells can be transfected with different ARE-engineering molecules and by using an ELISA kit to the relevant protein and assaying protein produced at 6 hour, 12 hour, 24 hour, 48 hour, and 7 days post-transfection.

In some embodiments, the nucleic acid encoding the membrane anchored immunomodulatory fusion proteins is operably linked to a nuclear factor of activated T-cells (NFAT) promoter or a functional portion or functional variant thereof “NFAT promoter” as used herein means one or more NFAT responsive elements linked to a minimal promoter of any gene expressed by T-cells. Preferably, the minimal promoter of a gene expressed by T-cells is a minimal human IL-2 promoter. The NFAT responsive elements may comprise, e.g., NFAT1, NFAT2, NFAT3, and/or NFAT4 responsive elements. The NFAT promoter (or functional portion or functional variant thereof) may comprise any number of binding motifs, e.g., at least two, at least three, at least four, at least five, or at least six, at least seven, at least eight, at least nine, at least ten, at least eleven, or up to twelve binding motifs.

TABLE 4
NFAT Promoter Related Sequences.

| Description | Amino Acid Sequence |
|---|---|
| 6X NFAT IL-2 | TCGAGGTCGACGGTATCGATAAGCTTGAT |
| minimal promoter | ATCGAATTAGGAGGAAAAACTGTTTCATA |
| | CAGAAGGCGTCAATTAGGAGGAAAAACTG |
| | TTTCATACAGAAGGCGTCAATTAGGAGGA |
| | AAAACTGTTTCATACAGAAGGCGTCAATT |
| | GGTCCCATCGAATTAGGAGGAAAAACTGT |
| | TTCATACAGAAGGCGTCAATTAGGAGGAA |
| | AAACTGTTTCATACAGAAGGCGTCAATTA |
| | GGAGGAAAAACTGTTTCATACAGAAGGCG |
| | TCAATTGGTCCCGGGACATTTTGACACCCC |
| | CATAATATTTTTCCAGAATTAACAGTATAA |
| | ATTGCATCTCTTGTTCAAGAGTTCCCTATC |
| | ACTCTCTTTAATCACTACTCACAGTAACCT |
| | CAACTCCTGGCCACC (SEQ ID NO: |
| | 255) |
| NFAT responsive | GGAGGAAAAACTGTTTCATACAGAAGGCG |
| element | T (SEQ ID NO: 256) |
| Human IL-2 | CATTTTGACACCCCCATAATATTTTTCCAG |
| Promoter | AATTAACAGTATAAATTGCATCTCTTGTTC |
| | AAGAGTTCCCTATCACTCTCTTTAATCACT |
| | ACTCACAGTAACCTCAACTCCTG (SEQ |
| | ID NO: 257) |

In a preferred embodiment, the NFAT promoter comprises six NFAT binding motifs. See, e.g., U.S. Pat. No. 8,556,882, which is incorporated by reference in its entirety and particularly for pertinent parts relating to NFAT promoters. In some embodiments, the NFAT promoter system controls expression of an immunomodulatory fusion protein that includes any of the immunomodulatory agents described herein. In certain embodiments, the immunomodulatory agent is selected from: IL-2, IL-12, IL-15, IL-18, IL-21, and a CD40 agonist (e.g., CD40L or agonistic anti-CD40 binding domain (e.g., an anti-CD40 scFv)) or a bioactive variant thereof. Exemplary nucleic acids encoding exemplary subject membrane anchored immunomodulatory fusion proteins operably linked to a NFAT promoter are depicted in Table 59. In some embodiments, the NFAT promoter system controls expression of an immunomodulatory fusion protein that includes IL-15. In some embodiments, the NFAT promoter system controls expression of an immunomodulatory fusion protein that includes IL-21. In some embodiments, the NFAT promoter system controls expression of an immunomodulatory fusion protein that includes IL-15 and IL-21.

In some embodiments, the invention provides TILs genetically modified to comprise DNA encoding an immunomodulatory fusion protein operably linked to the NFAT promoter. In some embodiments, the NFAT promoter controls expression of DNA encoding an immunomodulatory fusion protein that includes any of the immunomodulatory agents described herein. In certain embodiments, the immunomodulatory agent is selected from: IL-2, IL-12, IL-15, IL-18, IL-21, and a CD40 agonist (e.g., CD40L or agonistic anti-CD40 binding domain (e.g., an anti-CD40 scFv)) or a bioactive variant thereof. In some embodiments, the NFAT promoter controls expression of DNA encoding an immunomodulatory fusion protein that includes IL-15. In some embodiments, the NFAT promoter controls expression of DNA encoding an immunomodulatory fusion protein that includes IL-21. In some embodiments, the NFAT promoter controls expression of DNA encoding an immunomodulatory fusion protein that includes IL-15 and IL-21.

In some embodiments, the invention provides TILs genetically modified to comprise DNA encoding an immunomodulatory fusion protein operably linked to the NFAT promoter, wherein the immunomodulatory fusion protein is arranged according to the formula, from N- to C-terminus:

S1-IA1-L1-C1-L2-S2-IA2-L3-C2,

wherein S1 and S2 are each a signal peptide, IA1 and IA2 are each an immunomodulatory agent, L1-L3 are each a linker, and C1 and C2 are each a cell membrane anchor moiety. In some embodiments, IA1 and IA2 are the same immunomodulatory agent. In certain embodiments, IA1 and IA2 are different immunomodulatory agents. Suitable immunomodulatory agents including any of those described herein. In some embodiments, IA1 and IA2 are independently selected from IL-2, IL-12, IL-15, IL-18, IL-21, a CD40 agonist (e.g., CD40L or an agonistic anti-CD40 binding domain (e.g., an anti-CD40 scFv)) or a bioactive variant thereof. In some embodiments, IA1 and IA2 are selected from the group consisting of: IL-12 and IL-15, IL-15 and IL-18, CD40L and IL-15, IL-15 and IL-21, and IL-2 and IL-12. In some embodiments, IA1 and IA2 are independently selected from IL-15 and IL-21. In some embodiments, IA1 is IL-15 and IA2 is IL-21. In some embodiments, IA1 is IL-21 and IA2 is IL-15. In some embodiments, one or more of L1-L3 is a cleavable linker. In some embodiments two or more of L1-L3 are different linkers. In exemplary embodiments L2 is a cleavable linker. In some embodiments, L2 is furin cleavable P2A linker (e.g., SEQ ID NO:251). In some embodiments, C1 and C2 are independently transmembrane domains and/or transmembrane-intracellular domains. In certain embodiments C1 and C2 are the same. In exemplary embodiments, C1 and C2 are each a B7-1 transmembrane-intracellular domain (e.g., SEQ ID NO:239). In exemplary embodiments, C1 and C2 are different. Exemplary constructs that include two membrane anchored immunomodulatory fusion proteins according to the above formula are depicted in FIG. 36.

Nucleic acids encoding the subject membrane anchored immunomodulatory fusion proteins may be introduced into a population of TILs to produce transiently modified or genetically modified TILs that express the membrane anchored immunomodulatory fusion proteins using any suitable method. In some embodiments, nucleic acids encoding the membrane anchored immunomodulatory fusion proteins are introduced into a population of TILs using a microfluidic platform. In some embodiments, the microfluidic platform is a SQZ vector-free microfluidic platform. See, e.g., International Patent Application Publication Nos. WO 2013/059343A1, WO 2017/008063A1, or WO 2017/123663A1, or U.S. Patent Application Publication Nos. US 2014/0287509A1, US 2018/0201889A1, or US 2018/0245089A1, all of which are incorporated by reference herein in their entireties, and particularly for disclosures of microfluidic platforms for nucleic acid delivery. In the SQZ platform, the cell membranes of the cells for modification (e.g., TILs) are temporarily disrupted by microfluidic constriction, thereby allowing the delivery of nucleic acids encoding the membrane anchored immunomodulatory fusion proteins into the cells.

In some embodiments, the nucleic acid encoding the membrane anchored immunomodulatory fusion protein is mRNA and the microfluidic platform (e.g., SQZ vector-free microfluidic platform) is used to deliver the mRNA into TILs to produce transiently modified TILs. In some embodiments, the nucleic acid encoding the membrane anchored immunomodulatory fusion protein is DNA and the microfluidic platform (e.g., SQZ vector-free microfluidic platform) is used to deliver the DNA into TILs to produce stable genetically-modified TILs. The microfluidic platform (e.g., SQZ vector-free microfluidic platform) may be used to deliver the nucleic acid to any population of TILs produced during any steps of the Process 2A method disclosure herein (see, e.g., FIGS. 2-6) or GEN 3 method disclosure herein (see, e.g., FIG. 7) to produce the modified TILs. In some embodiments, the membrane anchored immunomodulatory fusion protein includes an IL-2, an IL-12, an IL-15, an IL-18, an IL-21, a CD40 agonist (e.g., CD40L or agonistic anti-CD40 binding domain (e.g., an anti-CD40 scFv)) or any combination thereof.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-15. In some embodiments, the second immunomodulatory agent is IL-2, IL-12, IL-18, IL-21, CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is CD40L. In some embodiments, the second immunomodulatory agent is IL-2, IL-12, IL-15, IL-18, IL-21, a CD40 agonist (e.g., CD40L or an agonistic anti-CD40 binding domain (e.g., an anti-CD40 scFv)) or a bioactive variant thereof.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-12. In some embodiments, the second immunomodulatory agent is IL-2, IL-15, IL-18, IL-21, CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-18. In some embodiments, the second immunomodulatory agent is IL-2, IL-12, IL-15, IL-21, CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-21. In some embodiments, the second immunomodulatory agent is IL-2, IL-12, IL-15, IL-18, CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-2. In some embodiments, the second immunomodulatory agent is IL-2, IL-12, IL-15, IL-18, IL-21, CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-2 and the second immunomodulatory agent is IL-12.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-2 and the second immunomodulatory agent is IL-15.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-2 and the second immunomodulatory agent is IL-18.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-2 and the second immunomodulatory agent is IL-21.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-2 and the second immunomodulatory agent is CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-12 and the second immunomodulatory agent is IL-15.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-12 and the second immunomodulatory agent is IL-18.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-12 and the second immunomodulatory agent is IL-21.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-12 and the second immunomodulatory agent is CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-15 and the second immunomodulatory agent is IL-18.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-15 and the second immunomodulatory agent is IL-21.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-15 and the second immunomodulatory agent is CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-18 and the second immunomodulatory agent is IL-21.

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-18 and the second immunomodulatory agent is CD40L or an anti-CD40 binding domain (e.g., an anti-CD40 scFv).

In exemplary embodiments, the modified TILs provided herein include two membrane anchored immunomodulatory fusion proteins that each include a different immunomodulatory agent (i.e., a first and second immunomodulatory agent), wherein the first immunomodulatory agents is IL-21 and the second immunomodulatory agent is CD40L or an anti-CD40 binding domain (e.g., an anti-CD40 scFv).

Additional membrane anchored immunomodulatory fusion proteins that can be included in the modified TILs provided herein are described in WO 2019/157130 A1, which is incorporated by reference in its entirety, particularly in relevant parts related to membrane anchored immunomodulatory fusion proteins.

Exemplary membrane anchored immunomodulatory fusion proteins to be included in the modified TILs provided herein are depicted in FIGS. 36 and 37, and Tables 58 and 59.

In some embodiments, the nucleic acid encoding any of the membrane anchored immunomodulatory fusion proteins described above is operably linked to an NFAT promoter or a functional portion or functional variant thereof.

### 2. Immunomodulatory Agent-TIL Antigen Binding Domain Fusion Proteins

In some embodiments, the modified TILs provided herein include immunomodulatory fusion proteins, wherein such fusion proteins include one or more immunomodulatory agents linked to a TIL antigen binding domain (ABD). In some embodiments, the one or more immunomodulatory agents is tethered to the TIL surface membrane upon TIL ABD binding to a TIL surface antigen.

The TIL antigen binding domain includes an antibody variable heavy domain (VH) and variable light domain (VL). In some embodiments, the TIL antigen binding domain is a full length antibody that includes a heavy chain according to the formula: VH-CH1-hinge-CH2-CH3 and a light chain according to the formula: VL-CL, wherein VH is a variable heavy domain; CH1, CH2, CH3 are heavy chain constant domains, VL is a variable light domain and CL is a light chain constant domain. In some embodiments, the TIL antigen binding domain is antibody fragment. In certain embodiments, TIL antigen binding domain is a Fab, Fab′, F(ab′)2, F(ab)2, variable fragment (Fv), domain antibody (dAb), or single chain variable fragment (scFv).

The TIL antigen binding domain can bind to any suitable TIL antigen that allows for the attachment of the immunomodulatory agent-TIL ABD fusion protein to the surface of the TIL. In exemplary embodiments, the TIL antigen binding domain is capable of binding to a TIL surface antigen. TIL surface antigens include, but are not limited to D16, CD45, CD4, CD8, CD3, CD11a, CD11b, CD11c, CD18, LFA-1, CD25, CD127, CD56, CD19, CD20, CD22, HLA-DR, CD197, CD38, CD27, CD137, OX40, GITR, CD56, CD196, CXCR3, CXCR4, CXCR5, CD84, CD229, CCR1, CCR5, CCR4, CCR6, CCR8, and/or CCR10. In some embodiments, the ABD binds to CD45. In particular embodiments, the ABD binds to a CD45 isoform selected from CD45RA, CD45RB, CD45RC or CD45Rβ. In particular embodiments, the ABD binds to a CD45 expressed primary on T cells.

In certain embodiments, the ABD binds to a checkpoint inhibitor. Exemplary checkpoint inhibitors include, but are not limited to PD-1, PD-L1, LAG-3, TIM-3 and CTLA-4 (see, e.g., Qin et al., Molecular Cancer 18:155 (2019)). In some embodiments, the ABD binds to a checkpoint inhibitor expressed on an immune effector cell (e.g., a T cell or NK cell). Exemplary anti-PD-1 antibodies are disclosed, for example, in U.S. Pat. Nos. 7,695,715, 7,332,582, 9,205,148, 8,686,119, 8,735,553, 7,488,802, 8,927,697, 8,993,731, and 9,102,727, which are incorporated by reference in their entireties, particularly in pertinent parts relating to anti-PD-1 antibodies. Exemplary anti PD-L1 antibodies are disclosed in U.S. Pat. Nos. 8,217,149, 8,779,108, 8,168,179, 8,552,154, 8,460,927, and 9,175,082, which are incorporated by reference in their entireties, particularly in pertinent parts relating to anti-PD-L1 antibodies. Exemplary anti-LAG-3 antibodies are disclosed in U.S. Pat. Nos. 9,244,059, 9,244,059, 9,505,839, which are incorporated by reference in their entireties, particularly in pertinent parts relating to anti-LAG-3 antibodies. Exemplary TIM-3 antibodies are disclosed in WO 2016/161270, U.S. Pat. Nos. 8,841,418, and 9,163,087, which are incorporated by reference in their entireties, particularly in pertinent parts relating to anti-TIM-3 antibodies. Exemplary CTLA-4 antibodies are disclosed in U.S. Pat. Nos. 6,984,720 and 7,411,057, which are incorporated by reference in their entireties, particularly in pertinent parts relating to anti-CTLA-4 antibodies.

In some embodiments, the ABD is an anti-CD45 antibody or a fragment thereof. In certain embodiments, the anti-CD45 antibody is a human anti-CD45 antibody, a humanized anti-CD45 antibody, or a chimeric anti-CD45 antibody. In exemplary embodiments, the ABD includes the vhCDR1-3 and vlCDR1-3 of anti-CD45 antibody BC8 (see US20170326259, incorporated by reference herein, particularly in relevant parts relating to anti-CD45 antibody sequences). In some embodiments, the ABD includes the variable heavy domain and variable domain of anti-CD45 antibody BC8. In some embodiments, the ABD includes the vhCDR1-3 and vlCDR1-3 or VH and VL of one of the following anti-CD45 antibodies: 10G10, UCHL1, 9.4, 4B2, or GAP8.3 (see Spertini et al., Immunology 113(4):441-452 (2004), Buzzi et al., Cancer Research 52:4027-4035 (1992)).

The immunomodulatory fusion proteins can be any suitable immunomodulatory agent including, for example, any of the immunomodulatory agents provided herein. In some embodiments, the immunomodulatory agent is an interleukin that promotes an anti-tumor response. In some embodiments, the immunomodulatory agent is a cytokine. In particular embodiments, the immunomodulatory agent is IL-2, IL-12, IL-15, IL-21 or a bioactive variant thereof. In certain embodiments, the fusion protein includes more than one immunomodulatory agents. In exemplary embodiments, the fusion protein includes 2, 3, 4, 5, 6, 7, 8, 9 or 10 different immunomodulatory agents.

The TIL antigen binding domain is attached to the immunomodulatory agent using any suitable linker. Suitable linkers include, but are not limited: a cleavable linker, a non-cleavable linker, a peptide linker, a flexible linker, a rigid linker, a helical linker, or a non-helical linker. In some embodiments, the linker is a peptide linker that optionally comprises Gly and Ser. Suitable linkers include linkers that are at least about 5, 6, 7, 8, 9, 10, 11, 12, 13, 14, 15, 16, 17, 18, 19, 20, 21, 22, 23, 24, 25, 26, 27, 28, 29 or 30 amino acid residues in length. In some embodiments, the linker is 5-10, 10-15, 15-20, 20-25, 25-30, 30-35, 35-40, 45-50, or 50-60 amino acids in length. In certain embodiments, the peptide linker is a (GGGS)ₙ or (GGGGS)ₙ linker, wherein n indicates the number of repeats of the motif and is an integer selected from 1-10. In some embodiments, the linker is an antibody hinge domain or a fragment thereof. In certain embodiments, the linker is a human immunoglobulin (Ig) hinge domain (e.g., an IgG1, IgG2, IgG3, IgG4, IgD, IgE, IgM or IgA hinge) or a fragment thereof. In some embodiments, the immunomodulatory agent is directly coupled to the TIL without a linker.

The immunomodulatory agent can be attached to the TIL antigen binding domain at a suitable position that does not impede binding of the fusion protein to a TIL. In some embodiments wherein the antigen binding domain is a full length antibody, the immunomodulatory agent is attached to the C-terminus or N-terminus of either the heavy chain or light chain. In some embodiments wherein the antigen binding domain is an scFv, the immunomodulatory agent is attached to the C-terminus or N-terminus of the variable heavy domain or variable light domain. In some embodiments wherein the antigen binding domain is an Fab, the immunomodulatory agent is attached to the C-terminus or N-terminus of the variable heavy domain or variable light domain. In some embodiments wherein the antigen binding domain is an Fab′, the immunomodulatory agent is attached to the C-terminus or N-terminus of the variable heavy domain or variable light domain. In some embodiments wherein the antigen binding domain is an Fab′2, the immunomodulatory agent is attached to the C-terminus or N-terminus of the variable heavy domain or variable light domain.

In some embodiments wherein the fusion protein includes two or more immunomodulatory agents, the immunomodulatory agents are attached to each other using any of the linkers described herein. In some embodiments, the two or more immunomodulatory agents are attached to different locations of the antigen binding domain. For example, in some embodiments wherein the TIL antigen binding domain is a full length antibody, the two or more immunomodulatory agents are attached at (i) different locations on the heavy chain (ii) different locations on the light chain or (iii) different locations on the heavy chain and/or light chain.

The subject immunomodulatory agent-TIL antigen binding domain fusion proteins can be made using any suitable method. In one aspect, provided herein are nucleic acids that encode the subject fusion proteins, expression vectors that include such nucleic acids, and host cells that include the expression vectors. Host cells that include the expression vectors encoding the subject fusion proteins are cultured under conditions for the expression of the fusion proteins and the fusion proteins are subsequently isolated and purified. In some embodiments, the purified fusion proteins are then incubated with a population of TILs under conditions that allow for the binding of the fusion protein to the TILs.

In some embodiments, the subject immunomodulatory agent-TIL antigen binding domain fusion proteins are attached to TILs produced during any of the steps of the Process 2A method disclosure herein (see, e.g., FIGS. 2-6). In exemplary embodiments, the fusion proteins are attached to TILs produced during any of the steps of the GEN 3 method disclosure herein (see, e.g., FIG. 7). In exemplary embodiments, the fusion proteins are attached to TILs produced from the first expansion step in the Process 2A method and/or the priming expansion step in the GEN 3 method provided herein. In exemplary embodiments, the fusion proteins are attached to TILs produced from the second expansion step in the Process 2A method and/or the rapid expansion step in the GEN 3 method provided herein. In some embodiments, the TILs are PD-1 positive TILs that have been preselected using the methods described herein.

Nucleic acids encoding the subject the subject immunomodulatory agent-TIL antigen binding domain fusion proteins may be introduced into a population of TILs to produce transiently modified or genetically modified TILs that express the subject immunomodulatory agent-TIL antigen binding domain fusion proteins using any suitable method. In some embodiments, nucleic acids encoding the subject immunomodulatory agent-TIL antigen binding domain fusion proteins are introduced into a population of TILs using a microfluidic platform. In some embodiments, the microfluidic platform is a SQZ vector-free microfluidic platform. See, e.g., International Patent Application Publication Nos. WO 2013/059343A1, WO 2017/008063A1, or WO 2017/123663A1, or U.S. Patent Application Publication Nos. US 2014/0287509A1, US 2018/0201889A1, or US 2018/0245089A1, all of which are incorporated by reference herein in their entireties, and particularly for disclosures of microfluidic platforms for nucleic acid delivery. In the SQZ platform, the cell membranes of the cells for modification (e.g., TILs) are temporarily disrupted by microfluidic constriction, thereby allowing the delivery of nucleic acids encoding the immunomodulatory agent-TIL antigen binding domain fusion protein into the cells.

In some embodiments, the nucleic acid encoding the subject immunomodulatory agent-TIL antigen binding domain fusion protein is mRNA and the microfluidic platform (e.g., SQZ vector-free microfluidic platform) is used to deliver the mRNA into TILs to produce transiently modified TILs. In some embodiments, the nucleic acid encoding the subject immunomodulatory agent-TIL antigen binding domain fusion protein is DNA and the microfluidic platform (e.g., SQZ vector-free microfluidic platform) is used to deliver the nucleic acid into TILs to produce stable genetically-modified TILs. The microfluidic platform (e.g., SQZ vector-free microfluidic platform) may be used to deliver the nucleic acid to any population of TILs produced during any steps of the Process 2A method disclosure herein (see, e.g., FIGS. 2-6) or GEN 3 method disclosure herein (see, e.g., FIG. 7) to produce the modified TILs. In some embodiments, the membrane anchored immunomodulatory fusion protein comprises an IL-2, an IL-12, an IL-15, an IL-21 or combinations thereof (e.g., IL-15 and IL-21).

Exemplary immunomodulatory agent-TIL antigen binding domain fusion proteins useful for the compositions and methods provided herein are further described, for example, in US Patent Application Publication No. 20200330514, which is incorporated by reference in its entirety and in pertinent parts related to immunomodulatory agent-TIL antigen binding domain fusion proteins.

### B. Nanoparticle Compositions

In some embodiments, the subject modified TILs provided herein include one or more nanoparticles, and those nanoparticles include one or more immunomodulatory agents. In some embodiments, the nanoparticles provided herein include a plurality of two or more proteins that are coupled to each other and/or a second component of the particle (e.g., reversibly linked through a degradable linker). In some embodiments, the proteins of the nanoparticles are present in a polymer or silica. In certain embodiments, the nanoparticle includes a nanoshell. The nanoparticles provided herein include one or more immunomodulatory agent. In some embodiments, the immunomodulatory agent is IL-2, IL-12, IL-15, IL-18, IL-21, a CD40 agonist (e.g., CD40L or agonistic anti-CD40 binding domain (e.g., an anti-CD40 scFv)) or a bioactive variant thereof. Nanoparticles are attached to the surface of the TIL using any suitable technique described herein.

Exemplary nanoparticles of use in the subject modified TILs provided herein include without limitation a liposome, a protein nanogel, a nucleotide nanogel, a polymer nanoparticle, or a solid nanoparticle. In some embodiments, the nanoparticle includes a liposome. In exemplary embodiments, the nanoparticle includes an immunomodulatory agent nanogel. In particular embodiments, the nanoparticle is an immunomodulatory agent nanogel with a plurality of immunomodulatory agents (e.g., cytokines) covalently linked to each other. In some embodiments, the nanoparticle includes at least one polymer, cationic polymer, or cationic block co-polymer on the nanoparticle surface. Exemplary nanoparticles that can be used in the compositions provided herein are disclosed, for example, in U.S. Pat. Nos. 9,283,184 and 9,603,944, each of which is incorporated by reference in its entirety and in pertinent parts related to nanoparticles.

The immunomodulatory agent can be any suitable immunomodulatory agent including, for example, any of the immunomodulatory agents provided herein. In some embodiments, the immunomodulatory agent is an interleukin that promotes an anti-tumor response. In some embodiments, the immunomodulatory agent is a cytokine. In particular embodiments, the immunomodulatory agent is IL-2, IL-12, IL-15, IL-21 or a bioactive variant thereof. In certain embodiments, the fusion protein includes more than one immunomodulatory agents. In exemplary embodiments, the fusion protein includes 2, 3, 4, 5, 6, 7, 8, 9 or 10 different immunomodulatory agents.

In some embodiments, the nanoparticle includes proteins that are covalently cross-linked to each other and/or a second component (e.g., a degradable linker). In some embodiments, the nanoparticle includes immunomodulatory agents that are reversibly linked through a degradable linker to a function group or polymer, or “reversibly modified.” In some embodiments, the nanoparticle is a nanogel that includes a plurality of immunomodulatory agents cross-linked to each other through a degradable linker (see U.S. Pat. No. 9,603,944). In exemplary embodiments, the protein of the nanogel are cross-linked to a polymer (e.g., polyethylene glycol (PEG)). In some embodiments, the polymers are cross-linked to the nanogel surface.

In some embodiments, the immunomodulatory agents of the nanoparticles are reversibly linked to each other through a degradable linker (e.g., a disulfide linker) such that under physiological conditions, the linker degrades, thereby releasing the immunomodulatory agent. In some embodiments, the immunomodulatory agents of the nanoparticles are reversibly linked to functional groups through a degradable linker such that under physiological conditions, the linker degrades and releases the immunomodulatory agent. Suitable degradable linkers include, but are not limited to: two N-hydroxysuccinimide (NHS) ester groups joined together by a flexible disulfide-containing linker that is sensitive to a reductive physiological environment; a hydrolysable linker that is sensitive to an acidic physiological environment (pH<7, for example, a pH of 4-5, 5-6, or 6- to less than 7, e.g., 6.9), or a protease sensitive linker that is sensitive to one or more enzymes present in biological media such as proteases in a tumor microenvironment such a matrix metalloproteases present in a tumor microenvironment or in inflamed tissue (e.g. matrix metalloproteinase 2 (MMP2) or matrix metalloproteinase 9 (MMP9)). A crosslinker sensitive to a reductive physiological environment is, for example, a crosslinker with disulfide containing linker that will react with amine groups on proteins by the presence of NHS groups which cross-link the proteins into high density protein nanogels. In some embodiments, the degradable cross-linker includes Bis[2-(N-succinimidyl-oxycarbonyloxy)ethyl]disulfide.

In some embodiments, the degradable linker includes at least one N-hydroxysuccinimide ester. In some embodiments, the degradable linker is a redox responsive linker. In some embodiments, the redox responsive linker includes a disulfide bond. In some embodiments, the degradable linkers provided herein include at least one N-hydroxysuccinimide ester, which is capable of reacting with proteins at neutral pH (e.g., about 6 to about 8, or about 7) without substantially denaturing the protein. In some embodiments, the degradable linkers are “redox responsive” linkers, meaning that they degrade in the presence of a reducing agent (e.g., glutathione, GSH) under physiological conditions (e.g., 20-40° C. and/or pH 4-8), thereby releasing intact protein from the compound to which it is reversibly linked. In some embodiments, the protein of the nanoparticles are linked to the degradable linker through a terminal or internal-NH₂ functional group (e.g., a side chain of a lysine).

In other embodiments, the proteins of the nanoparticle are linked by an enzyme-sensitive linker. Exemplary cleavable linker include those that are recognized by one of the following enzymes: metalloprotease MMP-1, MMP-2, MMP-3, MMP-8, MMP-9, MMP-14, plasmin, PSA, PSMA, CATHEPSIN D, CATHEPSIN K, CATHEPSIN S, ADAM10, ADAM12, ADAMTS, Caspase-1, Caspase-2, Caspase-3, Caspase-4, Caspase-5, Caspase-6, Caspase-7, Caspase-8, Caspase-9, Caspase-10, Caspase-11, Caspase-12, Caspase-13, Caspase-14, and TACE. See, e.g., U.S. Pat. Nos. 8,541,203 and 8,580,244, each of which is incorporated by reference in its entirety and in pertinent parts related to cleavable linkers.

In some embodiments, the nanoparticles are nanogels that include a monodispersed plurality of immunomodulatory agents (e.g., cytokines). In some embodiments, the immunomodulatory agents of the nanogels are cross-linked to polymer. In certain embodiments, the polymer is cross-linked to the surface of the nanogel. In particular embodiments, the nanogel includes: a) one more immunomodulatory agents reversibly and covalently cross-linked to each other through a degradable linker; and b) polymers cross-linked to surface exposed proteins of the nanogels. Such nanogels can be made by contacting the one or more immunomodulatory agents with a degradable linker under conditions that permit reversible covalent crosslinking of the immunomodulatory agents to each other through the degradable linker to form a plurality of immunomodulatory agent nanogels. Subsequently, the immunomodulatory agent nanogels are contacted with a polymer (e.g., polyethylene glycol) under conditions that permit crosslinking of the polymer to the immunomodulatory agents of the immunomodulatory agent nanogels, thereby producing a plurality of immunomodulatory agent-polymer nanogels.

In some embodiments, the nanoparticles include one or more polymers. Exemplary polymers include, but are not limited to: aliphatic polyesters, poly (lactic acid) (PLA), poly (glycolic acid) (PGA), co-polymers of lactic acid and glycolic acid (PLGA), polycarprolactone (PCL), polyanhydrides, poly(ortho)esters, polyurethanes, poly(butyric acid), poly(valeric acid), and poly(lactide-co-caprolactone), and natural polymers such as alginate and other polysaccharides including dextran and cellulose, collagen, chemical derivatives thereof, including substitutions, additions of chemical groups such as for example alkyl, alkylene, hydroxylations, oxidations, and other modifications routinely made by those skilled in the art), albumin and other hydrophilic proteins, zein and other prolamines and hydrophobic proteins, copolymers and mixtures thereof. In some embodiments, the immunomodulatory agents of the nanoparticles are linked to hydrophilic polymers. Exemplary hydrophilic polymers include, but are not limited to: polyethylene glycol (PEG), polyethylene glycol-b-poly lysine (PEG-PLL), and/or polyethylene glycol-b-poly arginine (PEG-PArg).

In some embodiments, the nanoparticle (e.g., nanogel) includes one or more polycations on its surface. Exemplary polycations for use in the subject nanoparticles include, but are not limited to, polylysine (poly-L-lysine and/or poly-D-lysine), poly(argininate glyceryl succinate) (PAGS, an arginine-based polymer), polyethyleneimine, polyhistidine, polyarginine, protamine sulfate, polyethylene gly col-b-polylysine (PEG-PLL), and polyethylene glycol-g-polylysine.

In some embodiments, the nanoparticle is associated with the TIL surface by electrostatic attraction to the TIL. In certain embodiments, the nanoparticle includes a ligand that has affinity for a surface molecule of the TIL (e.g., a surface protein, carbohydrate and/or lipid).

In particular embodiments, the nanoparticle includes an antigen binding domain that binds a TIL surface antigen as described herein. In some embodiments, the antigen binding domain is an antibody or fragment thereof. In exemplary embodiments, the TIL surface antigen is CD45, LFA-1, CD 11a (integrin alpha-L), CD 18 (integrin beta-2), CD11b, CD11c, CD25, CD8, or CD4. In exemplary embodiments, the antigen binding domain (ABD) is an anti-CD45 antibody or a fragment thereof. In certain embodiments, the anti-CD45 antibody is a human anti-CD45 antibody, a humanized anti-CD45 antibody, or a chimeric anti-CD45 antibody. In exemplary embodiments, the ABD includes the vhCDR1-3 and vlCDR1-3 of anti-CD45 antibody BC8 (see US20170326259, incorporated by reference herein, particularly in relevant parts relating to anti-CD45 antibody sequences). In some embodiments, the ABD includes the variable heavy domain and variable domain of anti-CD45 antibody BC8. In some embodiments, the ABD includes the vhCDR1-3 and vlCDR1-3 or VH and VL of one of the following anti-CD45 antibodies: 10G10, UCHL1, 9.4, 4B2, or GAP8.3 (see Spertini et al., Immunology 113(4):441-452 (2004), Buzzi et al., Cancer Research 52:4027-4035 (1992)). In such embodiments, the nanoparticles are attached to the surface of a population of TILs by incubating the TILs in the presence of the nanoparticles under conditions wherein the nanoparticles bind to the surface of the TILs.

In some embodiments, the nanoparticle is associated with the TIL cell surface by electrostatic attraction. In some embodiments the nanoparticle is covalently conjugated to the TIL. In other embodiments, the nanoparticle is not covalently conjugated to the TIL.

In some embodiments, the subject nanoparticles are attached to TILs produced during any of the steps of the Process 2A method disclosure herein (see, e.g., FIGS. 2-6). In exemplary embodiments, the subject nanoparticles are attached to TILs produced during any of the steps of the GEN 3 method disclosure herein (see, e.g., FIG. 7). In exemplary embodiments, the subject nanoparticles are attached to TILs produced from the first expansion step in the Process 2A method and/or the priming expansion step in the GEN 3 method provided herein. In exemplary embodiments, the subject nanoparticles are attached to TILs produced from the second expansion step in the Process 2A method and/or the rapid expansion step in the GEN 3 method provided herein. In some embodiments, the TILs are PD-1 positive TILs that have been preselected using the methods described herein.

Additional suitable nanoparticles for use in the modified TILs provided herein are disclosed in US Patent Application Publication No. US20200131239 and WO2020205808, each of which is incorporated by reference in its entirety and in relevant parts related to nanoparticles.

### C. Immunomodulatory Agents

The modified TILs provided herein may include one or more immunomodulatory agents attached to its surface. The immunomodulatory agents can be incorporated into any of the immunomodulatory fusion proteins described herein, including, for example, the membrane anchored immunomodulatory fusion proteins described herein. Any suitable immunomodulatory agent can be included in the subject modified TIL. In some embodiments, the immunomodulatory agent enhances TIL survival and/or anti-tumor activity once transferred to a patient. Exemplary immunomodulatory agents include, for example, cytokines. In some embodiments, the modified TIL includes one or more of the following cytokines: IL-2, IL-7, IL-12, IL-15, IL-18, IL-21, IL-23, IL-27, IL-4, IL-1α, IL-1β, IL-5, IFNγ, TNF α (TNFa), IFNα, IFNβ, GM-CSF, GCSF, or a biologically active variant thereof. In some embodiments, the immunomodulatory agent is a costimulatory molecule. In particular embodiments, the costimulatory molecule is one of the following: OX40, CD28, GITR, VISTA, CD40, CD3, or an agonist of CD137. In some embodiments, the immunomodulatory agent is a CD40 agonist (e.g., CD40L or an agonistic CD40 binding domain). Exemplary immunomodulatory agents are discussed in detailed further below.

### 1. IL-15

In some embodiments, the modified TILs provided herein include an IL-15. In exemplary embodiments, the IL-15 is included as part of an immunomodulatory fusion protein as described herein (e.g., a membrane anchored immunomodulatory fusion protein).

As used herein, “interleukin 15”, “IL-15” and “IL15” all refer to an interleukin that binds to and signals through a complex composed of an IL-15 specific receptor alpha chain (IL-15Rα), an IL-2/IL-15 receptor beta chain (CD122) and the common gamma chain (gamma-C, CD132) (e.g., Genbank Accession numbers: NM_00000585, NP_000576 and NP_751915 (human); and NM_001254747 and NP_001241676 (mouse)). IL-15 has been shown to stimulate T cell proliferation inside tumors. IL-15 also is able to extend the survivability of effector memory CD8+ T cells and is critical for the development of NK cells. Therefore, without being bound by any particular theory of operation, it is believed that modified TILs associated with an IL-15s described herein exhibit enhanced survival and/or anti-tumor effects.

IL-15 has a short half-life of less than 40 minutes in vivo. Modifications to IL-15 monomer can improve its in vivo pharmacokinetics in the treatment of cancers. These modifications have generally centered on improving the trans-presentation of IL-15 with the alpha subunit of IL-15 receptor, IL-15Rα. Such modifications include: 1) pre-association of IL-15 and its soluble receptor a-subunit-Fc fusion to form IL-15: IL-15Rα-Fc complex (see, e.g., Rubinstein et al., Proc Natl Acad Sci U.S.A. 103:9166-71 (2006)); 2) expression of the superagonist IL-15-sIL-15Rα-sushi protein (see, e.g., Bessard et al., Molecular cancer therapeutics 8: 2736-45 (2009)); and 3) pre-association of human IL-15 mutant IL-15N72D with IL-15Rα-Fc sushi-Fc fusion complex (see, e.g., Zhu et al., Journal of Immunology 183: 3598-6007 (2009)).

In some embodiments, the IL-15 associated with the modified TIL is a full length IL-15, a fragment or a variant of IL-15. In some embodiments, the IL-15 is a human IL-15 or a variant human IL-15. In exemplary embodiments, the IL-15 is a biological active human IL-15 variant. In some embodiments, the IL-15 includes a 1, 2, 3, 4, 5, 6 7, 8, 9, or 10 mutations as compared to a wild-type IL-15. In certain embodiments, the IL-15 includes an N72D mutation relative to a wild type human IL-15. In some embodiments, the variant IL-15 exhibits IL-15Rα binding activity.

In some embodiments, the immunomodulatory agent includes an IL-15 and an extracellular domain of an IL-15Rα. In certain embodiments, the immunomodulatory agent includes an IL-15 and an IL-15Rα fused to an Fc domain (IL-15Rα-Fc)

TABLE 5
IL-15 Related Sequences.

| Description | Amino Acid Sequence |
|---|---|
| Human IL-15 (N72D mutant) | NWVNVISDLKKIEDLIQSMHIDATLYTESDV |
| | HPSCKVTAMKCFLLELQVISLESGDASIHDT |
| | VENLIILANDSLSSNGNVTESGCKECEELEEK |
| | NIKEFLQSFVHIVQMFINTS (SEQ ID NO: 258) |
| Human IL-15R-alpha-Su/Fc domain | ITCPPPMSVEHADIWVKSYSLYSRERYICNSG |
| | FKRKAGTSSLTECVLNKATNVAHWTTPSLK |
| | CIREPKSCDKTHTCPPCPAPELLGGPSVFLFP |
| | PKPKDTLMISRTPEVTCVVVDVSHEDPEVKF |
| | NWYVDGVEVHNAKTKPREEQYNSTYRVVS |
| | VLTVLHQDWLNGKEYKCKVSNKALPAPIEK |
| | TISKAKGQPREPQVYTLPPSRDELTKNQVSL |
| | TCLVKGFYPSDIAVEWESNGQPENNYKTTPP |
| | VLDSDGSFFLYSKLTVDKSRWQQGNVFSCS |
| | VMHEALHNHYTQKSLSLSPGK (SEQ ID NO: |
| | 259) |
| Human IL-15R-alpha-Su (65 aa | ITCPPPMSVEHADIWVKSYSLYSRERYICNSG |
| truncated | FKRKAGTSSLTECVLNKATNVAHWTTPSLK |
| extracellular domain) | CIR (SEQ ID NO: 260) |
| Human IL-15 isoform 2 | MVLGTIDLCSCFSAGLPKTEANWVNVISDLK |
| | KIEDLIQSMHIDATLYTESDVHPSCKVTAMK |
| | CFLLELQVISLESGDASIHDTVENLIILANNSL |
| | SSNGNVTESGCKECEELEEKNIKEFLQSFVHI |
| | VQMFINTS (SEQ ID NO: 261) |
| Human IL-15 isoform 1 | MRISKPHLRSISIQCYLCLLLNSHFLTEAGIHV |
| | FILGCFSAGLPKTEANWVNVISDLKKIEDLIQ |
| | SMHIDATLYTESDVHPSCKVTAMKCFLLEL |
| | QVISLESGDASIHDTVENLIILANNSLSSNGN |
| | VTESGCKECEELEEKNIKEFLQSFVHIVQMFI |
| | NTS (SEQ ID NO: 262) |
| Human IL-15 (without signal | NWVNVISDLKKIEDLIQSMHIDATLYTESDV |
| peptide) | HPSCKVTAMKCFLLELQVISLESGDASIHDT |
| | VENLIILANNSLSSNGNVTESGCKECEELEEK |
| | NIKEFLQSFVHIVQMFINTS (SEQ ID NO: 263) |
| Human IL-15R-alpha | ITCPPPMSVEHADIWVKSYSLYSRERYICNSG |
| (85 aa truncated | FKRKAGTSSLTECVLNKATNVAHWTTPSLK |
| extracellular domain) | CIRDPALVHQRPAPPSTVTTAGV (SEQ ID |
| | NO: 264) |
| Human IL-15R-alpha | ITCPPPMSVEHADIWVKSYSLYSRERYICNSG |
| (182 aa truncated | FKRKAGTSSLTECVLNKATNVAHWTTPSLK |
| extracellular domain) | CIRDPALVHQRPAPPSTVTTAGVTPQPESLSP |
| | SGKEPAASSPSSNNTAATTAAIVPGSQLMPS |
| | KSPSTGTTEISSHESSHGTPSQTTAKNWELTA |
| | SASHQPPGVYPQGHSDTTVAISTST (SEQ ID |
| | NO: 265) |
| Human IL-15R-alpha | MAPRRARGCRTLGLPALLLLLLLRPPATRGI |
| | TCPPPMSVEHADIWVKSYSLYSRERYICNSG |
| | FKRKAGTSSLTECVLNKATNVAHWTTPSLK |
| | CIRDPALVHQRPAPPSTVTTAGVTPQPESLSP |
| | SGKEPAASSPSSNNTAATTAAIVPGSQLMPS |
| | KSPSTGTTEISSHESSHGTPSQTTAKNWELTA |
| | SASHQPPGVYPQGHSDTTVAISTSTVLLCGL |
| | SAVSLLACYLKSRQTPPLASVEMEAMEALP |
| | VTWGTSSRDEDLENCSHHL (SEQ ID NO: |
| | 266) |

In some embodiments the immunostimulatory protein is a superagonist IL-15 (IL-15SA) that includes a complex of human IL-15 and soluble human IL-15Rα. The combination of human IL-15 with soluble human IL-15Rα forms an IL-15 SA complex that possesses greater biological activity than human IL-15 alone. Soluble human IL-15Rα, as well as truncated versions of the extracellular domain, has been described in the art (Wei et al., 2001 J of Immunol. 167: 277-282). The amino acid sequence of human IL-15Rα is set forth in SEQ ID NO: 266. In some embodiments, the IL-15SA includes a complex of human IL-15 and soluble human. IL-15Rα comprising all or a portion of the extracellular domain, without the transmembrane or cytoplasmic domain. In some embodiments, the IL-15SA includes a complex of human IL-15 and soluble human IL-15Rα that includes the full extracellular domain or a truncated form of the extracellular domain which retains IL-15 binding activity.

In some embodiments, the IL-15SA includes a complex of human IL-15 and soluble human IL-15Rα that includes a truncated form of the extracellular domain which retains IL-15 binding activity. In some embodiments, the soluble human IL-15Rα includes amino acids 1-60, 1-61, 1-62, 1-63, 1-64 or 1-65 of human IL-15Rα. In some embodiments, the soluble human IL-15Rα includes amino acids 1-80, 1-81, 1-82, 1-83, 1-84 or 1-85 of human IL-15Rα. In some embodiments, the soluble human IL-15Rα includes amino acids 1-180, 1-181, or 1-182 of human IL-15Rα.

In some embodiments, the immunomodulatory agent is an IL-15SA comprising a complex of human IL-15 and soluble human IL-15Rα comprising a truncated form of the extracellular domain which retains IL-15 binding activity and comprises a Sushi domain. The Sushi domain of IL-15Rα is described in the art as approximately 60 amino acids in length and comprises 4 cysteines. (Wei et al., 2001). Truncated forms of soluble human IL-15Rα which retain IL-15 activity and comprise a Sushi domain are useful in IL-15SA of the present disclosure.

In some embodiments, the immunomodulatory agent includes a complex comprising soluble human IL-15Rα expressed as a fusion protein, such as an Fc fusion as described herein (e.g., human IgG1 Fc), with IL-15. In some embodiments, IL-15SA comprises a dimeric human IL-15Rα Fc fusion protein (e.g., human IgG1 Fc) complexed with two human IL-15 molecules.

In some embodiments, the immunomodulatory agent is an IL-15SA cytokine complex that includes an IL-15 molecule comprising an amino acid sequence set forth in SEQ ID NO: 258, SEQ ID NO: 261, SEQ ID NO:262, or SEQ ID NO:263. In some embodiments, an IL-15SA cytokine complex comprises a soluble IL-15Rα molecule comprising a sequence of SEQ ID NO:260, SEQ ID NO: 264 or SEQ ID NO:265.

In some embodiments, the immunomodulatory agent is an IL-15SA cytokine complex that includes a dimeric IL-15Rα Fc fusion protein complexed with two IL-15 molecules. In some embodiments, IL-15-SA comprises a dimeric IL-15RαSu (Sushi domain)/Fc (SEQ ID NO:259) and two IL-15N72D (SEQ TD NO:258) molecules (also known as ALT-803) as described in US20140134128, incorporated herein by reference. In some embodiments, the IL-15SA comprises a dimeric IL-15RαSu/Fc molecule (SEQ ID NO: 259) and two IL-15 molecules (SEQ ID NO: 261). In some embodiments, the IL-15SA comprises a dimeric IL-15RαSu/Fc molecule (SEQ ID NO: 259) and two IL-15 molecules (SEQ ID N0:262). In some embodiments, the IL-15SA comprises a dimeric IL-15RαSu/Fc molecule (SEQ ID NO:259) and two IL-15 molecules (SEQ ID NO:263).

In some embodiments, the IL-15SA includes a dimeric IL-15RαSu/Fc molecule (SEQ ID NO:259) and two IL-15 molecules having amino acid sequences selected from SEQ ID NO: 258, 258, 262, and 263.

In some embodiments, the IL-15SA includes a soluble IL-15Rα molecule (SEQ ID NO:260) and two IL-15 molecules (SEQ ID NO:258). In some embodiments, the IL-15SA comprises a soluble IL-15Rα molecule (SEQ ID NO:260) and two IL-15 molecules (SEQ ID N0:261). In some embodiments, the IL-15SA comprises a soluble IL-15Rα molecule (SEQ ID NO:260) and two IL-1 molecules (SEQ ID NO:262). In some embodiments, the IL-155A comprises a soluble IL-15Rα molecule (SEQ ID NO:260) and two IL-15 molecules (SEQ ID NO:263).

In some embodiments, the IL-15SA comprises a soluble IL-15Rα molecule (SEQ ID NO:264) and two IL-15 molecules (SEQ ID NO:258). In some embodiments, the IL-15SA comprises a soluble IL-15Rα molecule (SEQ ID NO:264) and two IL-15 molecules (SEQ ID N0:261). In some embodiments, the IL-15SA comprises a soluble IL-15Rα molecule (SEQ ID NO:264) and two IL-15 molecules (SEQ ID NO:262). In some embodiments, the IL-15SA comprises a soluble IL-15Rα molecule (SEQ ID NO:264) and two IL-15 molecules (SEQ ID N0:261).

In some embodiments, the IL-15SA includes a soluble IL-15Rα molecule (SEQ ID NO:265) and two IL-15 molecules (SEQ ID NO:258), In some embodiments, the IL-15SA comprises a soluble IL-15Rα molecule (SEQ ID NO:265) and two IL-15 molecules (SEQ ID NO:261). In some embodiments, the IL-15SA comprises a soluble IL-15Rα molecule (SEQ ID NO:265) and two IL-15 molecules (SEQ ID NO:262). In some embodiments, the IL-15SA comprises a soluble IL-15Rα molecule (SEQ ID NO:265) and two IL-15 molecules (SEQ ID NO:263).

In some embodiments, the IL-15SA comprises a dimeric IL-15RαSu/Fc (SEQ ID NO:269) molecule and two IL-15 molecules (SEQ ID NO:262). In some embodiments, the IL-15SA includes a dimeric IL-15RαSu/Fc (SEQ ID NO259) molecule and two IL-15 molecules (SEQ ID NO:263).

In some embodiments, the IL-15′A includes SEQ ID NO:259 and SEQ ID NO:260. In some embodiments IL-15SA comprises SEQ ID NO:261 or SEQ ID NO:262. In some embodiments the IL-15SA comprises SEQ ID NO:261 and SEQ ID NO:259. In some embodiments the IL-15SA comprises SEQ ID NO:262 and SEQ ID NO:259 In some embodiments the IL-15SA comprises SEQ ID NO:263 and SEQ ID NO:259 In some embodiments, the IL-15SA comprises SEQ ID NO:261 and SEQ ID NO:260. In some embodiments the IL-15SA comprises SEQ ID NO:262 and SEQ ID NO:260.

In some embodiments, the TIL compositions include an immunomodulatory fusion protein or nanoparticle composition that includes a IL-15 or a bioactive variant thereof. Exemplary fusion proteins that include IL-15 are depicted in FIGS. 36 and 37, and Tables 58 and 59.

In exemplary embodiments the TIL compositions provided herein includes a nucleic acid encoding an immunomodulatory fusion protein that includes an IL-15, wherein the nucleic acid is operably linked to a an NFAT promoter, an EF-1a promoter, an MND promoter, or an SSFV promoter, as described herein. Exemplary NFAT promoter-driven constructs for expression of immunomodulatory fusion proteins that include IL-15 are depicted in Table 59.

### 2. IL-12

In some embodiments, the modified TIL is associated with an IL-12 or a variant thereof. In exemplary embodiments, the IL-12 is included as part of an immunomodulatory fusion protein as described herein (e.g., a membrane anchored immunomodulatory fusion protein).

As used herein, “interleukin 12”, “IL-12” and “IL12” all refer to an interleukin that is a heterodimeric cytokine encoded by the IL-12A and IL-12B genes (Genbank Accession numbers: NM_000882 (IL-12A) and NM_002187 (IL-12B)). IL-12 is composed of a bundle of four alpha helices and is involved in the differentiation of native T cells into TH1 cells. It is encoded by two separate genes, IL-12A (p35) and IL-12B (p40). The active heterodimer (referred to as ‘p70’), and a homodimer of p40 are formed following protein synthesis. IL-12 binds to the IL-12 receptor, which is a heterodimeric receptor formed by IL-12R-β1 and IL-12R-β2. IL-12 is known as a T cell-stimulating factor that can stimulate the growth and function of T cells. In particular, IL-12 can stimulate the production of interferon gamma (IFN-γ), and tumor necrosis factor-alpha (TNF-α) from T cells and natural killer (NK) cells and reduce IL-4 mediated suppression of IFN-γ. IL-12 can further mediate enhancement of the cytotoxic activity of NK cells and CD8+ cytotoxic T lymphocytes. Moreover, IL-12 can also have anti-angiogenic activity by increasing production of interferon gamma, which in turn increases the production of the chemokine inducible protein-10 (IP-10 or CXCL10). IP-10 then mediates this anti-angiogenic effect. Thus, without being bound by any particular theory of operation, it is believed that IL-12 can increase the survivability and/or anti-tumor effects of the TIL compositions provided herein.

In some embodiments, the IL-12 associated with the modified TIL is a full length IL-12, a fragment or a variant of IL-12. In some embodiments, the IL-12 is a human IL-12 or a variant human IL-12. In exemplary embodiments, the IL-12 is a biological active human IL-12 variant. In some embodiments, the IL-12 includes a 1, 2, 3, 4, 5, 6 7, 8, 9, or 10 mutations as compared to a wild-type IL-12.

In some embodiments, the IL-12 included in the modified TIL compositions include an IL-12 p35 subunit or a variant thereof. In some embodiments, the IL-12 p35 subunit is a human IL-12 p35 subunit. In some embodiments, the IL-12 p35 subunit has the amino acid sequence In certain embodiments, the IL-12 included in the modified TIL compositions include an IL-12 p40 subunit or a variant thereof. In certain embodiments, the IL-12 is a single chain IL-12 polypeptide comprising an IL-12 p35 subunit attached to an IL-12 p40 subunit. Such IL-12 single chain polypeptides advantageously retain one or more of the biological activities of wildtype IL-12. In some embodiments, the single chain IL-12 polypeptide described herein is according to the formula, from N-terminus to C-terminus, (p40)-(L)-(p35), wherein “p40” is an IL-12 p40 subunit, “p35” is IL-12 p35 subunit and L is a linker. In other embodiments, the single chain IL-12 is according to the formula from N-terminus to C-terminus, (p35)-(L)-(p40). Any suitable linker can be used in the single chain IL-12 polypeptide including those described herein. Suitable linkers can include, for example, linkers having the amino acid sequence (GGGGS)x wherein x is an integer from 1-10. Other suitable linkers include, for example, the amino acid sequence GGGGGGS. Exemplary single chain IL-12 linkers than can be used with the subject single chain IL-12 polypeptides are also described in Lieschke et al., Nature Biotechnology 15: 35-40 (1997), which is incorporated herein in its entirety by reference and particularly for its teaching of IL-12 polypeptide linkers. In an exemplary embodiment, the single chain IL-12 polypeptide is a single chain human IL-12 polypeptide (i.e., it includes a human p35 and p40 IL-12 subunit).

TABLE 6
IL-12 Related Sequences.

| Description | Amino Acid Sequence |
|---|---|
| Human IL-12 p35 subunit | RNLPVATPDPGMFPCLHHSQNLLRAVSNML |
| | QKARQTLEFYPCTSEEIDHEDITKDKTSTVEA |
| | CLPLELTKNESCLNSRETSFITNGSCLASRKT |
| | SFMMALCLSSIYEDLKMYQVEFKTMNAKLL |
| | MDPKRQIFLDQNMLAVIDELMQALNFNSET |
| | VPQKSSLEEPDFYKTKIKLCILLHAFRIRAVTI |
| | DRVMSYLNAS (SEQ ID NO: 267) |
| Human IL-12 p40 subunit | IWELKKDVYVVELDWYPDAPGEMVVLTCD |
| | TPEEDGITWTLDQSSEVLGSGKTLTIQVKEF |
| | GDAGQYTCHKGGEVLSHSLLLLHKKEDGIW |
| | STDILKDQKEPKNKTFLRCEAKNYSGRFTC |
| | WWLTTISTDLTFSVKSSRGSSDPQGVTCGAA |
| | TLSAERVRGDNKEYEYSVECQEDSACPAAE |
| | ESLPIEVMVDAVHKLKYENYTSSFFIRDIIKP |
| | DPPKNLQLKPLKNSRQVEVSWEYPDTWSTP |
| | HSYFSLTFCVQVQGKSKREKKDRVFTDKTS |
| | ATVICRKNASISVRAQDRYYSSSWSEWASVP |
| | CS (SEQ ID NO: 268) |

In some embodiments, the TIL compositions include an immunomodulatory fusion protein or nanoparticle composition that includes a IL-12 or a bioactive variant thereof.

In exemplary embodiments the TIL compositions provided herein includes a nucleic acid encoding an immunomodulatory fusion protein that includes an IL-12, wherein the nucleic acid is operably linked to an NFAT promoter, an EF-1a promoter, an MND promoter, or an SSFV promoter, as described herein. See, e.g., U.S. Pat. No. 8,556,882, which is incorporated by reference in its entirety and particularly for pertinent parts relating to NFAT promoters for IL-12 expression. Exemplary fusion proteins that include IL-12 are depicted in FIGS. 36 and 37, and Table 58.

### 3. IL-18

In some embodiments, the modified TIL is associated with an IL-18 or a variant thereof. In exemplary embodiments, the IL-18 is included as part of an immunomodulatory fusion protein as described herein (e.g., a membrane anchored immunomodulatory fusion protein).

As used herein, “interleukin 18”, “IL-18,” “IL18,” “IGIF,” “IL-1g,” “interferon-gamma inducing factor,” and “IL1F4,” all refer to an interleukin that is a heterodimeric cytokine encoded by the IL-18 gene (e.g., Genbank Accession numbers: NM_001243211, NM_001562 and NM_001386420). IL-18, structurally similar to IL-1β, is a member of IL-1 superfamily of cytokines. This cytokine, which is expressed by many human lymphoid and nonlymphoid cells, has an important role in inflammatory processes. IL-18 in combination with IL-12 can activate cytotoxic T cells (CTLs), as well as natural killer (NK) cells, to produce IFN-γ and, therefore, contributes to tumor immunity. Thus, without being bound by any particular theory of operation, it is believed that IL-18 can enhance the anti-tumor effects of the TIL compositions provided herein.

In some embodiments, the IL-18 associated with the modified TIL is a full length IL-18, a fragment or a variant of IL-18. In some embodiments, the IL-18 is a human IL-18 or a variant human IL-18. In exemplary embodiments, the IL-18 is a biological active human IL-18 variant. In some embodiments, the IL-18 includes 1, 2, 3, 4, 5, 6 7, 8, 9, 10, 11, 12, 13, 14, 15, 16, 17, 18, 19, or 20 mutations as compared to a wild-type IL-18 (SEQ ID NO:269). In some embodiments, the bioactive variant is a decoy resistant IL-18 variant (“DR-IL18,” or “DR-IL-18”) that provides IL-18 signaling activity even in the presence of an inhibitory molecule such as IL-18 binding protein (IL-18BP). Exemplary IL-18 variants that can be included in the subject modified TILs described herein are shown below in Table 7. Additional IL-18 variants that can be included in the subject modified TILs are described in WO 2022/094473, which is incorporated by reference in its entirety and particular with respect to disclosures relating to variant DR-IL-18.

In some embodiments, the variant IL-18 includes a stability mutation pair selection from L C38S/C68S, C38S/C68G, C38S/C68A, C38S/C68D, and C38S/C68N [relative to the human wild-type IL-18—SEQ ID NO: 269]. In some embodiments, the variant IL-18 includes mutations at amino acid positions M51 (e.g., M51E, M51R, M51K, M51T, M51D, or M51N), K53 (e.g., K53G, K53S, K53T, or K53R), Q56 (e.g., Q56G, Q56R, Q56L, Q56E, Q56A, Q56V, or Q56K), D110 (e.g., D110S, DI 10N, D110G, D110K, D110H, D110Q, or D110E) and N111 (e.g., N111G, N111R, Ni 1 IS, Ni l ID, N111H, or N111Y) in addition to a stabilizing mutation pair selected from: C38S/C68S, C38S/C68G, C38S/C68A, C38S/C68D, and C38S/C68N [relative to the human wild type IL-18—SEQ ID NO: 269], In some such cases the stabilized IL-18 variant polypeptide additionally includes a mutation at amino acid position S105 (e.g., S105D, SI 05 A, S105N, S105R, S105D, or S105K); and in some cases, further includes mutations at amino acid positions P57 (e.g., P57A, P57L, P57G, or P57K) and M60 (e.g., M60L, M60R, M60K, or M60Q).

TABLE 7
IL-18 Related Sequences.

| Description | Amino Acid Sequence |
|---|---|
| Human IL-18 | YFGKLESKLSVIRNLNDQVLFIDQGNRPLFE |
| | DMTDSDCRDNAPRTIFIISMYKDSQPRGMAV |
| | TISVKCEKISTLSCENKIISFKEMNPPDNIKDT |
| | KSDIIFFQRSVPGHDNKMQFESSSYEGYFLA |
| | CEKERDLFKLILKKEDELGDRSIMFTVQNED |
| | (SEQ ID NO: 269) |
| Human IL-18 variant | YFGKLESKLSVIRNLNDQVLFIDQGNRPLFE |
| | DMTDSDCRDNAPRTIFIISKYSDSRARGLAV |
| | TISVKCEKISTLSCENKIISFKEMNPPDNIKDT |
| | KSDIIFFARVPGHGRKTQFESSSYEGYFLACE |
| | KERDLFKLILKKEDELGDRSIMFTVQNED |
| | (SEQ ID NO: 270) |
| Human IL-18 variant | YFGKLESKLSVIRNLNDQVLFIDQGNRPLFE |
| | DMTDSDSRDNAPRTIFIISKYSDSLARGLAVT |
| | ISVKSEKISTLSCENKIISFKEMNPPDNIKDTK |
| | SDIIFFQRDVPGHSRKMQFESSSYEGYFLACE |
| | KERDLFKLILKKEDELGDRSIMFTVQNED |
| | (SEQ ID NO: 331) |
| Human IL-18 variant | YFGKLESKLSVIRNLNDQVLFIDQGNR |
| | PLFEDMTDSDSRDNAPRTIFIISKYSDSL |
| | ARGLAVTISVKGEKISTLSCENKIISFKE |
| | MNPPDNIKDTKSDIIFFQRDVPGHSRK |
| | MQFESSSYEGYFLACEKERDLFKLILK |
| | KEDELGDRSIMFTVQNED (SEQ ID NO: |
| | 332) |
| Human IL-18 variant | YFGKLESKLSVIRNLNDQVLFIDQGNRPLFE |
| | DMTDSDSRDNAPRTIFIISKYSDSLARGLAVT |
| | ISVKAEKISTLSCENKIISFKEMNPPDNIKDTK |
| | SDIIFFQRDVPGHSRKMQFESSSYEGYFLACE |
| | KERDLFKLILKKEDELGDRSIMFTVQNED |
| | (SEQ ID NO: 333) |
| Human IL-18 variant | YFGKLESKLSVIRNLNDQVLFIDQGNRPLFE |
| | DMTDSDSRDNAPRTIFIISKYSDSLARGLAVT |
| | ISVKVEKISTLSCENKIISFKEMNPPDNIKDTK |
| | SDIIFFQRDVPGHSRKMQFESSSYEGYFLACE |
| | KERDLFKLILKKEDELGDRSIMFTVQNED |
| | (SEQ ID NO: 334) |
| Human IL-18 variant | YFGKLESKLSVIRNLNDQVLFIDQGNRPLFE |
| | DMTDSDSRDNAPRTIFIISKYSDSLARGLAVT |
| | ISVKDEKISTLSCENKIISFKEMNPPDNIKDTK |
| | SDIIFFQRDVPGHSRKMQFESSSYEGYFLACE |
| | KERDLFKLILKKEDELGDRSIMFTVQNED |
| | (SEQ ID NO: 335) |
| Human IL-18 variant | YFGKLESKLSVIRNLNDQVLFIDQGNRPLFE |
| | DMTDSDSRDNAPRTIFIISKYSDSLARGLAVT |
| | ISVKEEKISTLSCENKIISFKEMNPPDNIKDTK |
| | SDIIFFQRDVPGHSRKMQFESSSYEGYFLACE |
| | KERDLFKLILKKEDELGDRSIMFTVQNED |
| | (SEQ ID NO: 336) |
| Human IL-18 variant | YFGKLESKLSVIRNLNDQVLFIDQGNRPLFE |
| | DMTDSDSRDNAPRTIFIISKYSDSLARGLAVT |
| | ISVKNEKISTLSCENKIISFKEMNPPDNIKDTK |
| | SDIIFFQRDVPGHSRKMQFESSSYEGYFLACE |
| | KERDLFKLILKKEDEL (SEQ ID NO: 337) |
| Human IL-18 variant | YFGKLESKLSVIRNLNDQVLFIDQGNRPLFE |
| | DMTDSDCRDNAPRTIFIISTYKDSQPRGKAV |
| | TISVKCEKISTLSCENKIISFKEMNPPDNIKDT |
| | KSDIIFFQRDVPGHKHKMQFESSSYEGYFLA |
| | CEKERDLFKLILKKEDELGDRSIMFTVQNED |
| | (SEQ ID NO: 338) |
| Human IL-18 variant | YFGKLESKLSVIRNLNDQVLFIDQGNRPLFE |
| | DMTDSDCRDNAPRTIFIISTYKDKQPRAKAV |
| | TISVKCEKISTLSCENKIISFKEMNPPDNIKDT |
| | KSDIIFFQRDVPGHKHKMQFESSSYEGYFLA |
| | CEKERDLFKLILKKEDELGDRSIMFTIQNED |
| | (SEQ ID NO: 339) |
| Human IL-18 variant | RFGKLESKLSVIRNLNDQVLFIDQGNRPLFE |
| | DMTDSDCRDNAPRTIFIISTYKDSQPRGKAV |
| | TISVKCEKISTLSCENKIISFKEMNPPDNIKDT |
| | KSDIIFFQRDVPGHKHKMQFESSSYEGYFLA |
| | CEKERDLFKLILKKEDELGDRSIMFTVQNED |
| | (SEQ ID NO: 340) |
| Human IL-18 variant | RFGKLESKLSVIRNLNDQVLFIDQGNRPLFE |
| | DMTDSDCRDNAPRTIFIISTYRDSQPRGKAV |
| | TISVKCEKISTLSCENKIISFKEMNPPDNIKDT |
| | KSDIIFFQRNVPGHKYKMQFESSSYEGYFLA |
| | CEKERDLFKLILKKEDELGDRSIMFTVQNED |
| | (SEQ ID NO: 341) |
| Human IL-18 variant | YFGKLESQLSVIRNLNDQVLFIDQGNRPLFE |
| | DMTDSDCRDNAPRTIFIISTYKDKQPRTKAV |
| | TISVKCEKISTLSCENKIISFKEMNPPDNIKDT |
| | KSDIIFFQRRVPGHHNKMQFESSSYEGYFLA |
| | CEKERDLFKLILKKEDELGDRSIMFTVQKED |
| | (SEQ ID NO: 342) |
| Human IL-18 variant | YFGKLESRLSVIRNLNDQVLFIDQGNRPLFE |
| | DMTDSDCRDNAPRTIFIISKYKDKQPRAQAV |
| | TISVKCEKISTLSCENKIISFKEMNPPDNIKDT |
| | KSDIIFFQRDVPGHKHKMQFESSSYEGYFLA |
| | CEKERDLFKLILKKEDELGDRSIMFTIQNED |
| | (SEQ ID NO: 343) |
| Human IL-18 variant | YFGKLESRLSVIRNLNDQVLFIDQGNRPLFE |
| | DMTDSDCRDNAPRTIFIISDYKDKQPRAXAA |
| | VTISVKCEKISTLSCENKIISFKEMNPPDNIKD |
| | TKSDIIFFQRDVPGHKHKMQFESSSYEGYFL |
| | ACEKERDLFKLILKKEDELGDRSIMFTIQNED |
| | (SEQ ID NO: 344) |
| | X = any naturally occuring amino acid |
| Human IL-18 variant | YFGKHESKLSVIRNLNDQVLFIDQGNRPLFE |
| | DMTDSDCRDNAPRTIFIISTYRDSQPRGKAV |
| | TISVKCEKISTLSCENKIISFKEMNPPDNIKDT |
| | KSDIIFFQRDVPGHNNKMQFESSSYEGYFLA |
| | CEKERDLFKLILKKEDELGDRSIMFTTQNED |
| | (SEQ ID NO: 345) |
| Human IL-18 variant | YFGKIESKLSVIRNLNDQVLFIDQGNRPLFED |
| | MTDSDCRDNAPRTIFIISKYKDKQPRAQAVTI |
| | SVKCEKISTLSCENKIISFKEMNPPDNIKDTKS |
| | DIIFFQRKVPGHQHKMQFESSSYEGYFLACE |
| | KERDLFKLILKKEDELGDRSIMFTVQKED |
| | (SEQ ID NO: 346 |
| Human IL-18 variant | YFGKIESKLSVIRNLNDQVLFIDQGNRPLFED |
| | MTDSDCRDNAPRTIFIISTYKDRQPRGKAVTI |
| | SVKCEKISTLSCENKIISFKEMNPPDNIKDTKS |
| | DIIFFERDVPGHHHKMQFESSSYEGYFLACE |
| | KERDLFKLILKKEDELGDRSIMFTIQNED |
| | (SEQ ID NO: 347) |
| Human IL-18 variant | YFGKIESKLSVIRNLNDQVLFIDQGNRPLFED |
| | MTDSDCRDNAPRTIFIISTYKDKQPRGKAVTI |
| | SVKCEKISTLSCENKIISFKEMNPPDNIKDTKS |
| | DIIFFQRDVPGHKHKMQFESSSYEGYFLACE |
| | KERDLFKLILKKEDELGDRSIMFTTQHED |
| | (SEQ ID NO: 348) |
| Human IL-18 variant | YFGKIESKLSVIRNLNDQVLFIDQGNRPLFED |
| | MTDSDCRDNAPRTIFIISTYKDKQPRAKAVTI |
| | SVKCEKISTLSCENKIISFKEMNPPDNIKDTKS |
| | DIIFFQRRVPGHHHKMQFESSSYEGYFLACE |
| | KERDLFKLILKKEDELGDRSIMFTIQKED |
| | (SEQ ID NO: 349) |
| Human IL-18 variant | YFGKIESRLSVIRNLNDQVLFIDQGNRPLFED |
| | MTDSDCRDNAPRTIFIISTYKDKQPRGKAVTI |
| | SVKCEKISTLSCENKIISFKEMNPPDNIKDTKS |
| | DIIFFQRDVPGHDYKMQFESSSYEGYFLACE |
| | KERDLFKLILKKEDELGDRSIMFTIQKED |
| | (SEQ ID NO: 350) |
| Human IL-18 variant | YFGKYESRLSVIRNLNDQVLFIDQGNRPLFE |
| | DMTDSDCRDNAPRTIFIISTYRDSQPRGKAV |
| | TISVKCEKISTLSCENKIISFKEMNPPDNIKDT |
| | KSDIIFFQRDVPGHEHKMQFESSSYEGYFLA |
| | CEKERDLFKLILKKEDELGDRSIMFTVQKED |
| | (SEQ ID NO: 351) |
| Human IL-18 variant | HFGKYESKLSVIRNLNDQVLFIDQGNRPLFE |
| | DMTDSDCRDNAPRTIFIISTYRDSQPRGKAV |
| | TISVKCEKISTLSCENKIISFKEMNPPDNIKDT |
| | KSDIIFFQRDVPGHHNKMQFESSSYEGYFLA |
| | CEKERDLFKLILKKEDELGDRSIMFTVQKED |
| | (SEQ ID NO: 352) |
| Human IL-18 variant | RFGKLESKLSVIRNLNDQVLFIDQGNRPLFE |
| | DMTDSDCRDNAPRTIFIISTYRDSQPRAKAV |
| | TISVKCEKISTLSCENKIISFKEMNPPDNIKDT |
| | KSDIIFFQRDVPGHQHKMQFESSSYEGYFLA |
| | CEKERDLFKLILKKEDELGDRSIMFTAQKED |
| | (SEQ ID NO: 353) |
| Human IL-18 variant | RFGKLESRLSVIRNLNDQVLFIDQGNRPLFED |
| | MTDSDCRDNAPRTIFIISDYRDSQPRGRAVTI |
| | SVKCEKISTLSCENKIISFKEMNPPDNIKDTKS |
| | DIIFFKRNVPGHKYKMQFESSSYEGYFLACE |
| | KERDLFKLILKKEDELGDRSIMFTVQHED |
| | (SEQ ID NO: 354) |
| Human IL-18 variant | RFGKLESRLSVIRNLNDQVLFIDQGNRPLFED |
| | MTDSDCRDNAPRTIFIISNYRDSQPRGQAVTI |
| | SVKCEKISTLSCENKIISFKEMNPPDNIKDTKS |
| | DIIFFKRRVPGHNHKMQFESSSYEGYFLACE |
| | KERDLFKLILKKEDELGDRSIMFTVQKED |
| | (SEQ ID NO: 355) |
| Human IL-18 variant | RFGKLESRLSVIRNLNDQVLFIDQGNRPLFED |
| | MTDSDCRDNAPRTIFIISTYKDSQPRGKAVTI |
| | SVKCEKISTLSCENKIISFKEMNPPDNIKDTKS |
| | DIIFFQRDVPGHKHKMQFESSSYEGYFLACE |
| | KERDLFKLILKKEDELGDRSIMFTVQNED |
| | (SEQ ID NO: 356) |
| Human IL-18 variant | RFGKHESKLSVIRNLNDQVLFIDQGNRPLFE |
| | DMTDSDCRDNAPRTIFIISTYRDSQPRGKAV |
| | TISVKCEKISTLSCENKIISFKEMNPPDNIKDT |
| | KSDIIFFERNVPGHKYKMQFESSSYEGYFLA |
| | CEKERDLFKLILKKEDELGDRSIMFTVQNED |
| | (SEQ ID NO: 357) |
| Human IL-18 variant | RFGKLESRLSVIRNLNDQVLFIDQGNRPLFED |
| | MTDSDCRDNAPRTIFIISTYRDSQPRAKAVTI |
| | SVKCEKISTLSCENKIISFKEMNPPDNIKDTKS |
| | DIIFFERDVPGHQHKMQFESSSYEGYFLACE |
| | KERDLFKLILKKEDELGDRSIMFTIQXED |
| | (SEQ ID NO: 358) |
| | X = any naturally occuring amino acid |
| Human IL-18 variant | RFGKLESRLSVIRNLNDQVLFIDQGNRPLFED |
| | MTDSDCRDNAPRTIFIISTYRDSQPRTKAVTI |
| | SVKCEKISTLSCENKIISFKEMNPPDNIKDTKS |
| | DIIFFQRNVPGHHDKMQFESSSYEGYFLACE |
| | KERDLFKLILKKEDELGDRSIMFTVQHED |
| | (SEQ ID NO: 359) |
| Human IL-18 variant | RFGKLESRLSVIRNLNDQVLFIDQGNRPLFED |
| | MTDSDCRDNAPRTIFIISTYKDSQPRAKAVTI |
| | SVKCEKISTLSCENKIISFKEMNPPDNIKDTKS |
| | DIIFFERDVPGHQHKMQFESSSYEGYFLACE |
| | KERDLFKLILKKEDELGDRSIMFTIQKED |
| | (SEQ ID NO: 360) |
| Human IL-18 variant | RFGKHESKLSVIRNLNDQVLFIDQGNRPLFE |
| | DMTDSDCRDNAPRTIFIISTYRDSQPRGKAV |
| | TISVKCEKISTLSCENKIISFKEMNPPDNIKDT |
| | KSDIIFFERNVPGHKYKMQFESSSYEGYFLA |
| | CEKERDLFKLILKKEDELGDRSIMFTVQNED |
| | (SEQ ID NO: 361) |
| Human IL-18 variant | RFGKYESKLSVIRNLNDQVLFIDQGNRPLFE |
| | DMTDSDCRDNAPRTIFIISTYKDSQPRTKAVT |
| | ISVKCEKISTLSCDNKIISFKEMNPPDNIKDTK |
| | SDIIFFQRDVPGHKHKMQFESSSYEGYFLAC |
| | EKERDLFKLILKKEDELGDRSIMFTVQNED |
| | (SEQ ID NO: 362) |
| Human IL-18 variant | RFGKLESRLSVIRNLNDQVLFIDQGNRPLFED |
| | MTDSDCRDNAPRTIFIISTYRDSQPRTKAVTI |
| | SVKCEKISTLSCENKIISFKEMNPPDNIKDTKS |
| | DIIFFQRKVPGHNHKMQFESSSYEGYFLACE |
| | KERDLFKLILKKEDELGDRSIMFTVQKED |
| | (SEQ ID NO: 363) |
| Human IL-18 variant | YFGKLESKLSVIRNLNDQVLFIDQGNRPLFE |
| | DMTDSDCRDNAPRTIFIISEYKDSELRGRAVT |
| | ISVKCEKISTLSCENKIISFKEMNPPDNIKDTK |
| | SDIIFFPRAVPGHNRKVQFESSSYEGYFLACE |
| | KERDLFKLILKKEDELGDRSIMFTVQNED |
| | (SEQ ID NO: 364) |
| Human IL-18 variant | YFGKLESKLSVIRNLNDQVLFIDQGNRPLFE |
| | DMTDSDCRDNAPRTIFIISKYKDSAGRGLAV |
| | TISVKCEKISTLSCENKIISFKEMNPPDNIKDT |
| | KSDIIFFERDVPGHSNKVQFESSSYEGYFLAC |
| | EKERDLFKLILKKEDELGDRSIMFTVQNED |
| | (SEQ ID NO: 365) |
| Human IL-18 variant | YFGKLESKLSVIRNLNDQVLFIDQGNRPLFE |
| | DMTDSDCRDNAPRTIFIISKYGDSAARGLAV |
| | TISVKCEKISTLSCENKIISFKEMNPPDNIKDT |
| | KSDIIFFQRSVPGHKRKMQFESSSYEGYFLAC |
| | EKERDLFKLILKKEDELGDRSIMFTVQNED |
| | (SEQ ID NO: 366) |
| Human IL-18 variant | YFGKLESKLSVIRNLNDQVLFIDQGNRPLFE |
| | DMTDSDCRDNAPRTIFIISKYGDSRGRGLAV |
| | TISVKCEKISTLSCENKIISFKEMNPPDNIKDT |
| | KSDIIFFERDVPGHNSKRQFESSSYEGYFLAC |
| | EKERDLFKLILKKEDELGDRSIMFTVQNED |
| | (SEQ ID NO: 367) |
| Human IL-18 variant | YFGKLESKLSVIRNLNDQVLFIDQGNRPLFE |
| | DMTDSDCRDNAPRTIFIISKYGDSVPRGLAV |
| | TISVKCEKISTLSCENKIISFKEMNPPDNIKDT |
| | KSDIIFFARAVPGHSRKTQFESSSYEGYFLAC |
| | EKERDLFKLILKKEDELGDRSIMFTVQNED |
| | (SEQ ID NO: 368) |
| Human IL-18 variant | YFGKLESKLSVIRNLNDQVLFIDQGNRPLFE |
| | DMTDSDCRDNAPRTIFIISKYSDSGARGLAV |
| | TISVKCEKISTLSCENKIISFKEMNPPDNIKDT |
| | KSDIIFFARAVPGHGRKTQFESSSYEGYFLAC |
| | EKERDLFKLILKKEDELGDRSIMFTVQNED |
| | (SEQ ID NO: 369) |
| Human IL-18 variant | YFGKLESKLSVIRNLNDQVLFIDQGNRPLFE |
| | DMTDSDCRDNAPRTIFIISKYSDSKARGMAV |
| | TISVKCEKISTLSCENKIISFKEMNPPDNIKDT |
| | KSDIIFFARDVPGHSSKRQFESSSYEGYFLAC |
| | EKERDLFKLILKKEDELGDRSIMFTVQNED |
| | (SEQ ID NO: 370) |
| Human IL-18 variant | YFGKLESKLSVIRNLNDQVLFIDQGNRPLFE |
| | DMTDSDCRDNAPRTIFIISKYSDSLARGLAVT |
| | ISVKCEKISTLSCENKIISFKEMNPPDNIKDTK |
| | SDIIFFQRDVPGHSRKMQFESSSYEGYFLACE |
| | KERDLFKLILKKEDELGDRSIMFTVQNED |
| | (SEQ ID NO: 371) |
| Human IL-18 variant | YFGKLESKLSVIRNLNDQVLFIDQGNRPLFE |
| | DMTDSDCRDNAPRTIFIISKYSDSRARGLAV |
| | TISVKCEKISTLSCENKIISFKEMNPPDNIKDT |
| | KSDIIFFQRNVPGHGRKMQFESSSYEGYFLA |
| | CEKERDLFKLILKKEDELGDRSIMFTVQNED |
| | (SEQ ID NO: 372 |
| Human IL-18 variant | YFGKLESKLSVIRNLNDQVLFIDQGNRPLFE |
| | DMTDSDCRDNAPRTIFIISKYSDSRARGLAV |
| | TISVKCEKISTLSCENKIISFKEMNPPDNIKDT |
| | KSDIIFFARSVPGHGRKTQFESSSYEGYFLAC |
| | EKERDLFKLILKKEDELGDRSIMFTVQNED |
| | (SEQ ID NO: 373) |
| Human IL-18 variant | YFGKLESKLSVIRNLNDQVLFIDQGNRPLFE |
| | DMTDSDCRDNAPRTIFIISKYSDSRARGLAV |
| | TISVKCEKISTLSCENKIISFKEMNPPDNIKDT |
| | KSDIIFFARDVPGHSGKRQFESSSYEGYFLAC |
| | EKERDLFKLILKKEDELGDRSIMFTVQNED |
| | (SEQ ID NO: 374) |
| Human IL-18 variant | YFGKLESKLSVIRNLNDQVLFIDQGNRPLFE |
| | DMTDSDCRDNAPRTIFIISKYTDSRPRGLAVT |
| | ISVKCEKISTLSCENKIISFKEMNPPDNIKDTK |
| | SDIIFFERDVPGHSSKKQFESSSYEGYFLACE |
| | KERDLFKLILKKEDELGDRSIMFTVQNED |
| | (SEQ ID NO: 375) |
| Human IL-18 variant | YFGKLESKLSVIRNLNDQVLFIDQGNRPLFE |
| | DMTDSDCRDNAPRTIFIISKYTDSRARGMAV |
| | TISVKCEKISTLSCENKIISFKEMNPPDNIKDT |
| | KSDIIFFERDVPGHNDKRQFESSSYEGYFLAC |
| | EKERDLFKLILKKEDELGDRSIMFTVQNED |
| | (SEQ ID NO: 376) |
| Human IL-18 variant | YFGKLESKLSVIRNLNDQVLFIDQGNRPLFE |
| | DMTDSDCRDNAPRTIFIISRYKDSGKRGLAV |
| | TISVKCEKISTLSCENKIISFKEMNPPDNIKDT |
| | KSDIIFFRRSVPGHSRKVQFESSSYEGYFLAC |
| | EKERDLFKLILKKEDELGDRSIMFTVQNED |
| | (SEQ ID NO: 377) |
| Human IL-18 variant | YFGKLESKLSVIRNLNDQVLFIDQGNRPLFE |
| | DMTDSDCRDNAPRTIFIISKYGDSGARGLAV |
| | TISVKCEKISTLSCENKIISFKEMNPPDNIKDT |
| | KSDIIFFERDVPGHSGKVQFESSSYEGYFLAC |
| | EKERDLFKLILKKEDELGDRSIMFTVQNED |
| | (SEQ ID NO: 378) |
| Human IL-18 variant | YFGKLESKLSVIRNLNDQVLFIDQGNRPLFE |
| | DMTDSDCRDNAPRTIFIISKYGDSRPRGMAV |
| | TISVKCEKISTLSCENKIISFKEMNPPDNIKDT |
| | KSDIIFFQRAVPGHNRKMQFESSSYEGYFLA |
| | CEKERDLFKLILKKEDELGDRSIMFTVQNED |
| | (SEQ ID NO: 379 |
| Human IL-18 variant | YFGKLESKLSVIRNLNDQVLFIDQGNRPLFE |
| | DMTDSDCRDNAPRTIFIISKYSDSLARGLAVT |
| | ISVKCEKISTLSCENKIISFKEMNPPDNIKDTK |
| | SDIIFFQRDVPGHSRKMQFESSSYEGYFLACE |
| | KERDLFKLILKKEDELGDRSIMFTVQNED |
| | (SEQ ID NO: 380) |
| Human IL-18 variant | YFGKLESKLSVIRNLNDQVLFIDQGNRPLFE |
| | DMTDSDCRDNAPRTIFIISKYSDSRARGLAV |
| | TISVKCEKISTLSCENKIISFKEMNPPDNIKDT |
| | KSDIIFFARSVPGHGRKTQFESSSYEGYFLAC |
| | EKERDLFKLILKKEDELGDRSIMFTVQNE |
| | (SEQ ID NO: 381) |
| Human IL-18 variant | YFGKLESKLSVIRNLNDQVLFIDQGNRPLFE |
| | DMTDSDCRDNAPRTIFIISKYSDSRARGLAV |
| | TISVKCEKISTLSCENKIISFKEMNPPDNIKDT |
| | KSDIIFFQRNVPGHGRKMQFESSSYEGYFLA |
| | CEKERDLFKLILKKEDELGDRSIMFTVQNED |
| | (SEQ ID NO: 382) |
| Human IL-18 variant | YFGKLESKLSVIRNLNDQVLFIDQGNRPLFE |
| | DMTDSDCRDNAPRTIFIISKYGDSVPRGLAV |
| | TISVKCEKISTLSCENKIISFKEMNPPDNIKDT |
| | KSDIIFFARAVPGHSRKTQFESSSYEGYFLAC |
| | EKERDLFKLILKKEDELGDRSIMFTVQNED |
| | (SEQ ID NO: 383) |
| Human IL-18 variant | YFGKLESKLSVIRNLNDQVLFIDQGNRPLFE |
| | DMTDSDCRDNAPRTIFIISKYSDSRARGLAV |
| | TISVKCEKISTLSCENKIISFKEMNPPDNIKDT |
| | KSDIIFFARSVPGHGRKTQFESSSYEGYFLAC |
| | EKERDLFKLILKKEDELGDRSIMFTVQNED |
| | (SEQ ID NO: 384) |
| Human IL-18 variant | YFGKLESKLSVIRNLNDQVLFIDQGNRPLFE |
| | DMTDSDCRDNAPRTIFIISKYSDSRARGLAV |
| | TISVKCEKISTLSCENKIISFKEMNPPDNIKDT |
| | KSDIIFFARDVPGHSGKRQFESSSYEGYFLAC |
| | EKERDLFKLILKKEDELGDRSIMFTVQNED |
| | (SEQ ID NO: 385) |

In some embodiments, the TIL compositions include an immunomodulatory fusion protein or nanoparticle composition that includes a IL-18 or a bioactive variant thereof (e.g., any one of the IL-18 variants included in Table 7). Exemplary fusion proteins that include IL-18 are depicted in FIG. 36.

In exemplary embodiments the TIL compositions provided herein includes a nucleic acid encoding an immunomodulatory fusion protein that includes an IL-18, wherein the nucleic acid is operably linked to an NFAT promoter, an EF-1a promoter, an MND promoter, or an SSFV promoter, as described herein. Exemplary NFAT promoter-driven constructs for expression of immunomodulatory fusion proteins that include IL-21 are depicted in Table 59.

### 4. IL-21

In some embodiments, the modified TIL is associated with an IL-21 or a variant thereof. In exemplary embodiments, the IL-21 is included as part of an immunomodulatory fusion protein as described herein (e.g., a membrane anchored immunomodulatory fusion protein).

In certain embodiments, the cytokine-ABD includes an IL-21 molecule or fragment thereof. As used herein, “interleukin 21” “IL-21”, and “IL21” (e.g., Genbank Accession numbers: NM_001207006 and NP_001193935 (human); and NM_0001291041 and NP_001277970 (mouse)) all refer to a member of a cytokine that binds to IL-21 receptor and has potent regulatory effects on cells of the immune system, including natural killer (NK) cells and cytotoxic cells and binds to IL-21 receptor that can destroy virally infected or cancerous cells. Thus, without being bound by any particular theory of operation, it is believed that IL-21 can increase the survivability and/or anti-tumor effects of the TIL compositions provided herein.

In some embodiments, the IL-21 is a human IL-21. In some embodiments, the IL-21 associated with the modified TIL is a full length IL-21, a fragment or a variant of IL-21. In some embodiments, the IL-21 is a human IL-21 or a variant human IL-21. In exemplary embodiments, the IL-21 is a biological active human IL-21 variant. In some embodiments, the IL-21 includes a 1, 2, 3, 4, 5, 6 7, 8, 9, or 10 mutations as compared to a wild-type IL-21.

TABLE 8
IL-21 Related Sequences.

| Description | Amino Acid Sequence |
|---|---|
| Human IL-21 | QGQDRHMIRMRQLIDIVDQLKNYVNDLVPE |
| | FLPAPEDVETNCEWSAFSCFQKAQLKSANT |
| | GNNERIINVSIKKLKRKPPSTNAGRRQKHRL |
| | TCPSCDSYEKKPPKEFLERFKSLLQKMIHQH |
| | LSSRTHGSEDS (SEQ ID NO: 271) |

In some embodiments, the TIL compositions include an immunomodulatory fusion protein or nanoparticle composition that includes a IL-21 or a bioactive variant thereof. Exemplary fusion proteins that include IL-21 are depicted in FIGS. 36 and 37, and Tables 58 and 59.

In exemplary embodiments the TIL compositions provided herein includes a nucleic acid encoding an immunomodulatory fusion protein that includes an IL-21, wherein the nucleic acid is operably linked to an NFAT promoter, an EF-1a promoter, an MND promoter, or an SSFV promoter, as described herein.

### 5. IL-2

In some embodiments, the modified TIL is associated with an IL-2 or a variant thereof. In exemplary embodiments, the IL-2 is included as part of an immunomodulatory fusion protein as described herein (e.g., a membrane anchored immunomodulatory fusion protein).

In certain embodiments, the cytokine-ABD includes an IL-2 molecule or fragment thereof. As used herein, “interleukin 2” “IL-2”, “IL2,” and “TCGF” (e.g., Genbank Accession numbers: NM_000586 and NP_000577 (human) all refer to a member of a cytokine that binds to IL-2 receptor. IL-2 enhances activation-induced cell death (AICD). IL-2 also promotes the differentiation of T cells into effector T cells and into memory T cells when the initial T cell is also stimulated by an antigen, thus helping the body fight off infections. Together with other polarizing cytokines, IL-2 stimulates naive CD4+ T cell differentiation into Th1 and Th2 lymphocytes and impedes differentiation into Th17 and follicular Th lymphocytes. IL-2 also increases the cell killing activity of both natural killer cells and cytotoxic T cells. Thus, without being bound by any particular theory of operation, it is believed that IL-2 can increase the survivability and/or anti-tumor effects of the TIL compositions provided herein.

In some embodiments, the IL-2 is a human IL-2. In some embodiments, the IL-2 associated with the modified TIL is a full length IL-2, a fragment or a variant of IL-2. In some embodiments, the IL-2 is a human IL-2 or a variant human IL-2. In exemplary embodiments, the IL-2 is a biological active human IL-2 variant. In some embodiments, the IL-2 includes a 1, 2, 3, 4, 5, 6 7, 8, 9, or 10 mutations as compared to a wild-type IL-2.

TABLE 9
IL-2 Related Sequences.

| Description | Amino Acid Sequence |
|---|---|
| Human IL-2 | MYRMQLLSCIALSLALVTNSAPTSSSTKKTQ |
| | LQLEHLLLDLQMILNGINNYKNPKLTRMLTF |
| | KFYMPKKATELKHLQCLEEELKPLEEVLNL |
| | AQSKNFHLRPRDLISNINVIVLELKGSETTFM |
| | CEYADETATIVEFLNRWITFCQSIISTLT |
| | (SEQ ID NO: 272) |

In some embodiments, the TIL compositions include an immunomodulatory fusion protein or nanoparticle composition that includes a IL-2 or a bioactive variant thereof. Exemplary fusion proteins that include IL-2 are depicted in FIGS. 36 and 37.

In exemplary embodiments the TIL compositions provided herein includes a nucleic acid encoding an immunomodulatory fusion protein that includes an IL-2, wherein the nucleic acid is operably linked to an NFAT promoter, an EF-1a promoter, an MND promoter, or an SSFV promoter, as described herein.

### 6. CD40 Agonists

In some embodiments, the modified TIL is associated with CD40 agonist. In exemplary embodiments, the CD40 agonist is included as part of an immunomodulatory fusion protein as described herein (e.g., a membrane anchored immunomodulatory fusion protein).

Cluster of differentiation 40, CD40, is a costimulatory protein found on antigen-presenting cells (APCs) and is required for APC activation. The binding of CD40L (CD154) on T helper cells to CD40 activates antigen presenting cells (e.g., dendritic cells) and induces a variety of downstream effects. Without being by any particular theory of operation, it is believed that the addition of one or more immunomodulatory agents that activate CD40 on antigen presenting cells (i.e., CD40 agonists) can enhance the anti-tumor effects of the TIL compositions provided herein. CD40 agonists, include, for example, CD40L and antibody or antibody fragments thereof (e.g., an scFv) that agonistically binds CD40. In some embodiments, the TIL compositions include an immunomodulatory fusion protein or nanoparticle composition that includes a CD40L or a bioactive variant thereof. In some embodiments, the TIL composition includes an immunomodulatory fusion protein that includes an agonistic anti-CD40 binding domain (e.g., an scFv). Exemplary CD40 agonist sequences are depicted in the table below.

CD40 agonist activity can be measured using any suitable method known in the art. Ligation of CD40 on DC, for example, induces increased surface expression of costimulatory and MHC molecules, production of proinflammatory cytokines, and enhanced T cell triggering. CD40 ligation on resting B cells increases antigen-presenting function and proliferation. In exemplary embodiments, the CD40 agonist is capable of activating human dendritic cells.

In some embodiments, the TIL composition includes an agonistic anti-CD40 binding domain having the VH and VL sequences of an anti-CD40 scFv depicted in Table 10 or a bioactive variant thereof. In some embodiments, the anti-CD40 binding domain includes a VH sequence that is at least about 80%, 85%, 90%, 91%, 92%, 93%, 94%, 95%, 96%, 97%, 98%, 99% identical to the VH sequence depicted in Table 10. In some embodiments, the agonistic anti-CD40 binding domain includes a VH sequence that includes 1, 2, 3, 4, 5, 6, 7, 8, 9, 10, 11, 12, 13, 14, 15, 16, 17, 18, 19, or 20 amino acid substitutions as compared to the VH sequence depicted in Table 10. In some embodiments, the anti-CD40 binding domain includes a VL sequence that is at least about 80%, 85%, 90%, 91%, 92%, 93%, 94%, 95%, 96%, 97%, 98%, 99% identical to the VL sequence depicted in Table 10. In some embodiments, the anti-CD40 binding domain includes a VL sequence that includes 1, 2, 3, 4, 5, 6, 7, 8, 9, 10, 11, 12, 13, 14, 15, 16, 17, 18, 19, or 20 amino acid substitutions as compared to the VL sequence depicted in Table 10. In exemplary embodiments, the anti-CD40 binding domain is an anti-CD40 scFv selected from SEQ ID NOs:276, 279, 282, and 285 in Table 10.

In some embodiments, the anti-CD40 binding domain is a variant of an anti-CD40 scFv in Table 10 that is capable of binding to human CD40. In exemplary embodiments, the variant anti-CD40 scFv is least about 75%, 80%, 85%, 90%, 95%, or 99% identical to an anti-CD40 scFv selected from SEQ ID NOs:276, 279, 282, and 285 in Table 10.

Assessment of CD40 binding domain binding can be measured using any suitable assay known in the art, including, but not limited to: a Biacore, surface plasmon resonance (SPR) and/or BLI (biolayer interferometry, e.g., Octet assay) assay.

Additional CD40 binding domains (VH and VLs) that are useful as immunomodulatory agents include those described in U.S. Pat. Nos. 6,838,261, 6,843,989, 7,338,660, 8,7778,345, which are incorporated by reference herein, particularly with respect to teachings of anti-CD40 antibodies and VH, VL and CDR sequences.

In some embodiments, the CD40 agonist is a CD40 ligand (CD40L). In exemplary embodiments, the CD40L is human CD40L (SEQ ID NO:270). In some embodiments, the CD40L is a variant of a human CD40L that is at least about 75%, 80%, 85%, 90%, 95%, or 99% identical to SEQ ID NO:253. In some embodiments, the CD40L is a variant of a human CD40L that includes 1, 2, 3, 4, 5, 6, 7, 8, 9, 10, 11, 12, 13, 14, 15, 16, 17, 18, 19, or 20 amino acid substitutions as compared to SEQ ID NO:273.

Exemplary fusion proteins that include CD40 agonists are depicted in FIGS. 36 and 37.

In exemplary embodiments the TIL compositions provided herein includes a nucleic acid encoding an immunomodulatory fusion protein that includes a CD40 agonist, wherein the nucleic acid is operably linked to a NFAT promoter, as described herein.

TABLE 10
CD40 Agonist Related Sequences.

| Description | Amino Acid Sequence |
|---|---|
| Human CD40L | MIETYNQTSPRSAATGLPISMKIFMYLLTVFL |
| | ITQMIGSALFAVYLHRRLDKIEDERNLHEDF |
| | VFMKTIQRCNTGERSLSLLNCEEIKSQFEGFV |
| | KDIMLNKEETKKENSFEMQKGDQNPQIAAH |
| | VISEASSKTTSVLQWAEKGYYTMSNNLVTL |
| | ENGKQLTVKRQGLYYIYAQVTFCSNREASS |
| | QAPFIASLCLKSPGRFERILLRAANTHSSAKP |
| | CGQQSIHLGGVFELQPGASVFVNVTDPSQVS |
| | HGTGFTSFGLLKL |
| | (SEQ ID NO: 273) |
| Anti-human CD40 VH | QVQLVESGGGVVQPGRSLRLSCAASGFSFSS |
| (Sotigalimab) | TYVCWVRQAPGKGLEWIACIYTGDGTNYSA |
| | SWAKGRFTISKDSSKNTVYLQMNSLRAEDT |
| | AVYFCARPDITYGFAINFWGPGTLVTVSS |
| | (SEQ ID NO: 274) |
| Anti-human CD40 VL | DIQMTQSPSSLSASVGDRVTIKCQASQSISSR |
| (Sotigalimab) | LAWYQQKPGKPPKLLIYRASTLASGVPSRFS |
| | GSGSGTDFTLTISSLQPEDVATYYCQCTGYGI |
| | SWPIGGGTKVEIK (SEQ ID NO: 275) |
| Anti-human CD40 scFv | QVQLVESGGGVVQPGRSLRLSCAASGFSFSS |
| (Sotigalimab) | TYVCWVRQAPGKGLEWIACIYTGDGTNYSA |
| | SWAKGRFTISKDSSKNTVYLQMNSLRAEDT |
| | AVYFCARPDITYGFAINFWGPGTLVTVSSGG |
| | GGSGGGGSGGGGSGGGGSDIQMTQSPSSLS |
| | ASVGDRVTIKCQASQSISSRLAWYQQKPGKP |
| | PKLLIYRASTLASGVPSRFSGSGSGTDFTLTIS |
| | SLQPEDVATYYCQCTGYGISWPIGGGTKVEI |
| | K (SEQ ID NO: 276) |
| Anti-human CD40 VH | QLVESGGGLVQPGGSLRLSCAASGYSFTGY |
| (Dacetuzumab) | YIHWVRQAPGKGLEWVARVIPNAGGTSYN |
| | QKFKGRFTLSVDNSKNTAYLQMNSLRAEDT |
| | AVYYCAREGIYWWGQGTLVTVSS (SEQ ID |
| | NO: 277) |
| Anti-human CD40 VL | DIQMTQSPSSLSASVGDRVTITCRSSQSLVHS |
| (Dacetuzumab) | NGNTFLHWYQQKPGKAPKLLIYTVSNRFSG |
| | VPSRFSGSGSGTDFTLTISSLQPEDFATYFCS |
| | QTTHVPWTFGQGTKVEIK (SEQ ID NO: 278) |
| Anti-human CD40 scFv | QLVESGGGLVQPGGSLRLSCAASGYSFTGY |
| (Dacetuzumab) | YIHWVRQAPGKGLEWVARVIPNAGGTSYN |
| | QKFKGRFTLSVDNSKNTAYLQMNSLRAEDT |
| | AVYYCAREGIYWWGQGTLVTVSSGGGGSG |
| | GGGSGGGGSGGGGSDIQMTQSPSSLSASVG |
| | DRVTITCRSSQSLVHSNGNTFLHWYQQKPG |
| | KAPKLLIYTVSNRFSGVPSRFSGSGSGTDFTL |
| | TISSLQPEDFATYFCSQTTHVPWTFGQGTKV |
| | EIK (SEQ ID NO: 279) |
| Anti-CD40 VH | QVQLVESGGGVVQPGRSLRLSCAASGFTFSS |
| (Lucatutuzumab) | YGMHWVRQAPGKGLEWVAVISYEESNRYH |
| | ADSVKGRFTISRDNSKITLYLQMNSLRTEDT |
| | AVYYCARDGGIAAPGPDYWGQGTLVTVSS |
| | (SEQ ID NO: 280) |
| Anti-CD40 VL | DIVMTQSPLSLTVTPGEPASISCRSSQSLLYS |
| (Lucatutuzumab) | NGYNYLDWYLQKPGQSPQVLISLGSNRASG |
| | VPDRFSGSGSGTDFTLKISRVEAEDVGVYYC |
| | MQARQTPFTFGPGTKVDIR (SEQ ID NO: 281) |
| Anti-CD40 scFv | QVQLVESGGGVVQPGRSLRLSCAASGFTFSS |
| (Lucatutuzumab) | YGMHWVRQAPGKGLEWVAVISYEESNRYH |
| | ADSVKGRFTISRDNSKITLYLQMNSLRTEDT |
| | AVYYCARDGGIAAPGPDYWGQGTLVTVSS |
| | GGGGSGGGGSGGGGSGGGGSDIVMTQSPLS |
| | LTVTPGEPASISCRSSQSLLYSNGYNYLDWY |
| | LQKPGQSPQVLISLGSNRASGVPDRFSGSGS |
| | GTDFTLKISRVEAEDVGVYYCMQARQTPFT |
| | FGPGTKVDIR (SEQ ID NO: 282) |
| Anti-CD40 VH | QVQLVQSGAEVKKPGASVKVSCKASGYTFT |
| (Selicrelumab) | GYYMHWVRQAPGQGLEWMGWINPDSGGT |
| | NYAQKFQGRVTMTRDTSISTAYMELNRLRS |
| | DDTAVYYCARDQPLGYCTNGVCSYFDYWG |
| | QGTLVTVSS (SEQ ID NO: 283) |
| Anti-CD40 VL | DIQMTQSPSSVSASVGDRVTITCRASQGIYS |
| (Selicrelumab) | WLAWYQQKPGKAPNLLIYTASTLQSGVPSR |
| | FSGSGSGTDFTLTISSLQPEDFATYYCQQANI |
| | FPLTFGGGTKVEIK (SEQ ID NO: 284) |
| Anti-CD40 scFv | QVQLVQSGAEVKKPGASVKVSCKASGYTFT |
| (Selicrelumab) | GYYMHWVRQAPGQGLEWMGWINPDSGGT |
| | NYAQKFQGRVTMTRDTSISTAYMELNRLRS |
| | DDTAVYYCARDQPLGYCTNGVCSYFDYWG |
| | QGTLVTVSSGGGGSGGGGSGGGGSGGGGSD |
| | IQMTQSPSSVSASVGDRVTITCRASQGIYSW |
| | LAWYQQKPGKAPNLLIYTASTLQSGVPSRFS |
| | GSGSGTDFTLTISSLQPEDFATYYCQQANIFP |
| | LTFGGGTKVEIK (SEQ ID NO: 285) |

### IV. Gene-Editing Processes

### A. Overview: TIL Expansion+Gene-Editing+Transient Gene-Editing

In some embodiments of the present invention directed to methods for expanding TIL populations, the methods comprise one or more steps of gene-editing at least a portion of the TILs in order to enhance their therapeutic effect. As used herein, “gene-editing,” “gene editing,” and “genome editing” refer to a type of genetic modification in which DNA is permanently modified in the genome of a cell, e.g., DNA is inserted, deleted, modified or replaced within the cell's genome. In some embodiments, gene-editing causes the expression of a DNA sequence to be silenced (sometimes referred to as a gene knockout) or inhibited/reduced (sometimes referred to as a gene knockdown). In other embodiments, gene-editing causes the expression of a DNA sequence to be enhanced (e.g., by causing over-expression). In accordance with embodiments of the present invention, gene-editing technology is used to enhance the effectiveness of a therapeutic population of TILs.

A method for expanding tumor infiltrating lymphocytes (TILs) into a therapeutic population of TILs may be carried out in accordance with any embodiment of the methods described herein (e.g., an exemplary TIL expansion method known as process 2A is described below), wherein the method further comprises gene-editing at least a portion of the TILs. According to additional embodiments, a method for expanding TILs into a therapeutic population of TILs is carried out in accordance with any embodiment of the methods described in U.S. Pat. No. 10,517,894, U.S. Patent Application Publication No. 2020/0121719 A1, or U.S. Pat. No. 10,894,063, which are incorporated by reference herein in their entireties, wherein the method further comprises gene-editing at least a portion of the TILs. Thus, some embodiments of the present invention provides a therapeutic population of TILs that has been expanded in accordance with any embodiment described herein, wherein at least a portion of the therapeutic population has been gene-edited, e.g., at least a portion of the therapeutic population of TILs that is transferred to the infusion bag is permanently gene-edited.

In some embodiments of the present invention directed to methods for expanding TIL populations, the methods comprise one or more steps of introducing into at least a portion of the TILs nucleic acids, e.g., mRNAs, for transient expression of an immunomodulatory protein, e.g., an immunomodulatory fusion protein comprising an immunomodulatory protein fused to a membrane anchor, in order to produce modified TILs with (i) reduced dependence on cytokines in when expanded in culture and/or (ii) an enhanced therapeutic effect. As used herein, “transient gene-editing”, “transient gene editing”, “transient phenotypic alteration,” “transient phenotypic modification”, “temporary phenotypic alteration,” “temporary phenotypic modification”, “transient cellular change”, “transient cellular modification”, “temporary cellular alteration”, “temporary cellular modification”, “transient expression”, “transient alteration of expression”, “transient alteration of protein expression”, “transient modification”, “transitory phenotypic alteration”, “non-permanent phenotypic alteration”, “transiently modified”, “temporarily modified”, “non-permanently modified”, “transiently altered”, “temporarily altered”, grammatical variations of any of the foregoing, and any expressions of similar meaning, refer to a type of cellular modification or phenotypic change in which nucleic acid (e.g., mRNA) is introduced into a cell, such as transfer of nucleic acid into a cell by electroporation, calcium phosphate transfection, viral transduction, etc., and expressed in the cell (e.g., expression of an immunomodulatory protein, such as an immunomodulatory fusion protein comprising an immunomodulatory protein fused to a membrane anchor) in order to effect a transient or non-permanent phenotypic change in the cell, such as the transient display of membrane-anchored immunomodulatory fusion protein on the cell surface. In accordance with embodiments of the present invention, transient phenotypic alteration technology is used to reduce dependence on cytokines in the expansion of TILs in culture and/or enhance the effectiveness of a therapeutic population of TILs.

In some embodiments, a microfluidic platform is used for intracellular delivery of nucleic acids encoding the immunomodulatory fusion proteins provided herein. In some embodiments, the microfluidic platform is a SQZ vector-free microfluidic platform. The SQZ platform is capable of delivering nucleic acids and proteins, to a variety of primary human cells, including T cells (Sharei et al. PNAS 2013, as well as Sharei et al. PLOS ONE 2015 and Greisbeck et al. J. Immunology vol. 195, 2015). In the SQZ platform, the cell membranes of the cells for modification (e.g., TILs) are temporarily disrupted by microfluidic constriction, thereby allowing the delivery of nucleic acids encoding the immunomodulatory fusion proteins into the cells. Such methods as described in International Patent Application Publication Nos. WO 2013/059343A1, WO 2017/008063A1, or WO 2017/123663A1, or U.S. Patent Application Publication Nos. US 2014/0287509A1, US 2018/0201889A1, or US 2018/0245089A1 can be employed with the present invention for delivering nucleic acids encoding the subject immunomodulatory fusion proteins to a population of TILs. In some embodiments, the delivered nucleic acid allows for transient protein expression of the immunomodulatory fusion proteins in the modified TILs. In some embodiments, the SQZ platform is used for stable incorporation of the delivered nucleic acid encoding the immunomodulatory fusion protein into the TIL cell genome.

In some embodiments of the present invention, a transposon/transposase system is used for both delivery of a gene-editing system and transiently altering protein expression. In some embodiments, the transposon/transposase system comprises a piggyBac transposons and transposases or piggyBac-like transposons and transposases; a sleeping beauty (SB) transposon and transposase; or a Helraiser transposon and transposase. In some embodiments, a transposon/transposase system (e.g., piggyBac transposons and transposases or piggyBac-like transposons and transposases; a sleeping beauty (SB) transposon and transposase; or a Helraiser transposon and transposase) a is used for both for expression of any one of the immunomodulatory fusion proteins provided herein (e.g., a membrane anchord immunonomodulatory fusion protein), and altering expression of an immune checkpoint gene. In some embodiments, the immune checkpoint is one of the following: PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

### B. Timing of Gene-Editing/Transient Phenotypic Alteration During TIL Expansion

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2, and optionally OKT-3 (e.g., OKT-3 may be present in the culture medium beginning on the start date of the expansion process), to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing the cell culture medium of the second population of TILs with additional IL-2, optionally OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting the therapeutic population of TILs obtained from step (d), wherein the transition from step (d) to step (e) occurs without opening the system;
(f) transferring the harvested TIL population from step (e) to an infusion bag, wherein the transfer from step (e) to (f) occurs without opening the system; and
(g) at any time during the method prior to the transfer to the infusion bag in step (f), gene-editing at least a portion of the TIL cells to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

As stated in step (g) of the embodiments described above, the gene-editing process may be carried out at any time during the TIL expansion method prior to the transfer to the infusion bag in step (f), which means that the gene editing may be carried out on TILs before, during, or after any of the steps in the expansion method; for example, during any of steps (a)-(f) outlined in the method above, or before or after any of steps (a)-(e) outlined in the method above. According to certain embodiments, TILs are collected during the expansion method (e.g., the expansion method is “paused” for at least a portion of the TILs), and the collected TILs are subjected to a gene-editing process, and, in some cases, subsequently reintroduced back into the expansion method (e.g., back into the culture medium) to continue the expansion process, so that at least a portion of the therapeutic population of TILs that are eventually transferred to the infusion bag are permanently gene-edited. In some embodiments, the gene-editing process may be carried out before expansion by activating TILs, performing a gene-editing step on the activated TILs, and expanding the gene-edited TILs according to the processes described herein. In some embodiments, nucleic acids for gene editing are delivered to the TILs using a microfluidic platform. In some embodiments, the microfluidic platform is a SQZ vector-free microfluidic platform.

In some embodiments, the gene-editing process is carried out after the first TIL expansion step. In some embodiments, the gene-editing process is carried out after the first TIL expansion step and before the second expansion step. In some embodiments, the gene-editing process is carried out after the TILs are activated. In some embodiments, the gene-editing process is carried out after the first expansion step and after the TILs are activated, but before the second expansion step. In some embodiments, the gene-editing process is carried out after the first expansion step and after the TILs are activated, and the TILs are rested after gene-editing and before the second expansion step. In some embodiments, the TILs are rested for about 1 to 2 days after gene-editing and before the second expansion step. In some embodiments, the TILs are activated by exposure to an anti-CD3 agonist and an anti-CD28 agonist. In some embodiments, the anti-CD3 agonist is an anti-CD3 agonist antibody and the anti-CD28 agonist is an anti-CD28 agonist antibody. In some embodiments, the anti-CD3 agonist antibody is OKT-3. In some embodiments, the TILs are activated by exposure to anti-CD3 agonist antibody- and anti-CD28 agonist antibody-conjugated beads. In some embodiments, the anti-CD3 agonist antibody- and anti-CD28 agonist antibody-conjugated beads are the TransAct™ product of Miltenyi. In some embodiments, the gene-editing process is carried out by viral transduction. In some embodiments, the gene-editing process is carried out by retroviral transduction. In some embodiments, the gene-editing process is carried out by lentiviral transduction. In some embodiments, the immunomodulatory composition is a membrane anchored immunomodulatory fusion protein. In some embodiments, the immunomodulatory fusion protein comprises IL-15. In some embodiments, the immunomodulatory fusion protein comprises IL-21. In some embodiments, the immunomodulatory composition comprises two or more different membrane bound fusion proteins. In some embodiments, the immunomodulatory composition comprises a first immunomodulatory protein comprising IL-15 and a second immunomodulatory fusion protein comprising IL-21. In some embodiments, the TILs are gene-edited to express the immunomodulatory composition under the control of an NFAT promoter. In some embodiments, the TILs are gene-edited to express an immunomodulatory fusion protein comprising IL-15 under the control of an NFAT promoter. In some embodiments, the TILs are gene-edited to express an immunomodulatory fusion protein comprising IL-21 under the control of an NFAT promoter. In some embodiments, the TILs are gene-edited to express a first immunomodulatory fusion protein comprising IL-15 and a second immunomodulatory fusion protein comprising IL-21 under the control of an NFAT promoter.

In some embodiments, the gene-editing process is carried out by viral transduction. In some embodiments, the gene-editing process is carried out by retroviral transduction. In some embodiments, the gene-editing process is carried out by lentiviral transduction.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2, and optionally OKT-3 (e.g., OKT-3 may be present in the culture medium beginning on the start date of the expansion process), to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) gene-editing at least a portion of the TIL cells in the second population of TILs to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells;
(e) performing a second expansion by supplementing the cell culture medium of the second population of TILs with additional IL-2, optionally OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(f) harvesting the therapeutic population of TILs obtained from step (d), wherein the transition from step (d) to step (e) occurs without opening the system; and
(g) transferring the harvested TIL population from step (e) to an infusion bag,
   wherein the transfer from step (e) to (f) occurs without opening the system. In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, the TILs are rested after the gene-editing step and before the second expansion step. In some embodiments, the TILs are rested for about 1 to 2 days after the gene-editing step and before the second expansion step. In some embodiments, the TILs are activated by exposure to an anti-CD3 agonist and an anti-CD28 agonist. In some embodiments, the anti-CD3 agonist is an anti-CD3 agonist antibody and the anti-CD28 agonist is an anti-CD28 agonist antibody. In some embodiments, the anti-CD3 agonist antibody is OKT-3. In some embodiments, the TILs are activated by exposure to anti-CD3 agonist antibody- and anti-CD28 agonist antibody-conjugated beads. In some embodiments, the anti-CD3 agonist antibody- and anti-CD28 agonist antibody-conjugated beads are the TransAct™ product of Miltenyi. In some embodiments, the gene-editing process is carried out by viral transduction. In some embodiments, the gene-editing process is carried out by retroviral transduction of the TILs, optionally for about 2 days. In some embodiments, the gene-editing process is carried out by lentiviral transduction of the TILs, optionally for about 2 days. In some embodiments, the immunomodulatory composition is a membrane anchored immunomodulatory fusion protein. In some embodiments, the immunomodulatory fusion protein comprises IL-15. In some embodiments, the immunomodulatory fusion protein comprises IL-21. In some embodiments, the immunomodulatory composition comprises two or more different membrane bound fusion proteins. In some embodiments, the immunomodulatory composition comprises a first immunomodulatory protein comprising IL-15 and a second immunomodulatory fusion protein comprising IL-21. In some embodiments, the TILs are gene-edited to express the immunomodulatory composition under the control of an NFAT promoter. In some embodiments, the TILs are gene-edited to express an immunomodulatory fusion protein comprising IL-15 under the control of an NFAT promoter. In some embodiments, the TILs are gene-edited to express an immunomodulatory fusion protein comprising IL-21 under the control of an NFAT promoter. In some embodiments, the TILs are gene-edited to express a first immunomodulatory fusion protein comprising IL-15 and a second immunomodulatory fusion protein comprising IL-21 under the control of an NFAT promoter.

It should be noted that alternative embodiments of the expansion process may differ from the method shown above; e.g., alternative embodiments may not have the same steps (a)-(g), or may have a different number of steps. Regardless of the specific embodiment, the gene-editing process may be carried out at any time during the TIL expansion method. For example, alternative embodiments may include more than two expansions, and it is possible that gene-editing may be conducted on the TILs during a third or fourth expansion, etc.

According to other embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2, and optionally OKT-3 (e.g., OKT-3 may be present in the culture medium beginning on the start date of the expansion process), to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing the cell culture medium of the second population of TILs with additional IL-2, optionally OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting the therapeutic population of TILs obtained from step (d), wherein the transition from step (d) to step (e) occurs without opening the system;
(f) transferring the harvested TIL population from step (e) to an infusion bag, wherein the transfer from step (e) to (f) occurs without opening the system; and
(g) at any time during the method prior to the transfer to the infusion bag in step (f), introducing a transient phenotypic alteration in at least a portion of the TIL cells to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, nucleic acids for transient phenotypic alteration are delivered to the TILs using a microfluidic platform. In some embodiments, the microfluidic platform is a SQZ vector-free microfluidic platform.

As stated in step (g) of the embodiments described above, the transient phenotypic alteration process may be carried out at any time during the TIL expansion method prior to the transfer to the infusion bag in step (f), which means that the transient phenotypic alteration may be carried out on TILs before, during, or after any of the steps in the expansion method; for example, during any of steps (a)-(f) outlined in the method above, or before or after any of steps (a)-(e) outlined in the method above. According to certain embodiments, TILs are collected during the expansion method (e.g., the expansion method is “paused” for at least a portion of the TILs), and the collected TILs are subjected to a transient modification process, and, in some cases, subsequently reintroduced back into the expansion method (e.g., back into the culture medium) to continue the expansion process, so that at least a portion of the therapeutic population of TILs that are eventually transferred to the infusion bag are transiently altered to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the transient cellular modification process may be carried out before expansion by activating TILs, performing a transient phenotypic alteration step on the activated TILs, and expanding the modified TILs according to the processes described herein.

It should be noted that alternative embodiments of the expansion process may differ from the method shown above; e.g., alternative embodiments may not have the same steps (a)-(g), or may have a different number of steps. Regardless of the specific embodiment, the transient cellular modification process may be carried out at any time during the TIL expansion method. For example, alternative embodiments may include more than two expansions, and it is possible that transient cellular modification process may be conducted on the TILs during a third or fourth expansion, etc.

According to some embodiments, the gene-editing process is carried out on TILs from one or more of the first population, the second population, and the third population. For example, gene-editing may be carried out on the first population of TILs, or on a portion of TILs collected from the first population, and following the gene-editing process those TILs may subsequently be placed back into the expansion process (e.g., back into the culture medium). Alternatively, gene-editing may be carried out on TILs from the second or third population, or on a portion of TILs collected from the second or third population, respectively, and following the gene-editing process those TILs may subsequently be placed back into the expansion process (e.g., back into the culture medium). According to other embodiments, gene-editing is performed while the TILs are still in the culture medium and while the expansion is being carried out, i.e., they are not necessarily “removed” from the expansion in order to conduct gene-editing.

According to some embodiments, the transient cellular modification process is carried out on TILs from one or more of the first population, the second population, and the third population. For example, transient cellular modification may be carried out on the first population of TILs, or on a portion of TILs collected from the first population, and following the gene-editing process those transiently modified TILs may subsequently be placed back into the expansion process (e.g., back into the culture medium). Alternatively, transient cellular modification may be carried out on TILs from the second or third population, or on a portion of TILs collected from the second or third population, respectively, and following the transient cellular modification process those modified TILs may subsequently be placed back into the expansion process (e.g., back into the culture medium). According to other embodiments, transient cellular modification is performed while the TILs are still in the culture medium and while the expansion is being carried out, i.e., they are not necessarily “removed” from the expansion in order to effect transient cellular modification.

According to other embodiments, the gene-editing process is carried out on TILs from the first expansion, or TILs from the second expansion, or both. For example, during the first expansion or second expansion, gene-editing may be carried out on TILs that are collected from the culture medium, and following the gene-editing process those TILs may subsequently be placed back into the expansion method, e.g., by reintroducing them back into the culture medium.

According to other embodiments, the transient cellular modification process is carried out on TILs from the first expansion, or TILs from the second expansion, or both. For example, during the first expansion or second expansion, transient cellular modification may be carried out on TILs that are collected from the culture medium, and following the transient cellular modification process those modified TILs may subsequently be placed back into the expansion method, e.g., by reintroducing them back into the culture medium.

According to other embodiments, the gene-editing process is carried out on at least a portion of the TILs after the first expansion and before the second expansion. For example, after the first expansion, gene-editing may be carried out on TILs that are collected from the culture medium, and following the gene-editing process those TILs may subsequently be placed back into the expansion method, e.g., by reintroducing them back into the culture medium for the second expansion.

According to other embodiments, the transient cellular modification process is carried out on at least a portion of the TILs after the first expansion and before the second expansion. For example, after the first expansion, transient cellular modification may be carried out on TILs that are collected from the culture medium, and following the transient cellular modification process those modified TILs may subsequently be placed back into the expansion method, e.g., by reintroducing them back into the culture medium for the second expansion.

According to alternative embodiments, the gene-editing process is carried out before step (c) (e.g., before, during, or after any of steps (a)-(b)), before step (d) (e.g., before, during, or after any of steps (a)-(c)), before step (e) (e.g., before, during, or after any of steps (a)-(d)), or before step (f) (e.g., before, during, or after any of steps (a)-(e)).

According to alternative embodiments, the transient cellular modification process is carried out before step (c) (e.g., before, during, or after any of steps (a)-(b)), before step (d) (e.g., before, during, or after any of steps (a)-(c)), before step (e) (e.g., before, during, or after any of steps (a)-(d)), or before step (f) (e.g., before, during, or after any of steps (a)-(e)).

It should be noted with regard to OKT-3, according to certain embodiments, that the cell culture medium may comprise OKT-3 beginning on the start day (Day 0), or on Day 1 of the first expansion, such that the gene-editing or transient cellular modification is carried out on TILs after they have been exposed to OKT-3 in the cell culture medium on Day 0 and/or Day 1. According to other embodiments, the cell culture medium comprises OKT-3 during the first expansion and/or during the second expansion, and the gene-editing or transient cellular modification is carried out before the OKT-3 is introduced into the cell culture medium. Alternatively, the cell culture medium may comprise OKT-3 during the first expansion and/or during the second expansion, and the gene-editing or transient cellular modification is carried out after the OKT-3 is introduced into the cell culture medium.

It should also be noted with regard to a 4-1BB agonist, according to certain embodiments, that the cell culture medium may comprise a 4-1BB agonist beginning on the start day (Day 0), or on Day 1 of the first expansion, such that the gene-editing or transient cellular modification is carried out on TILs after they have been exposed to a 4-1BB agonist in the cell culture medium on Day 0 and/or Day 1. According to other embodiments, the cell culture medium comprises a 4-1BB agonist during the first expansion and/or during the second expansion, and the gene-editing or transient cellular modification is carried out before the 4-1BB agonist is introduced into the cell culture medium. Alternatively, the cell culture medium may comprise a 4-1BB agonist during the first expansion and/or during the second expansion, and the gene-editing or transient cellular modification is carried out after the 4-1BB agonist is introduced into the cell culture medium.

It should also be noted with regard to IL-2, according to certain embodiments, that the cell culture medium may comprise IL-2 beginning on the start day (Day 0), or on Day 1 of the first expansion, such that the gene-editing or transient cellular modification is carried out on TILs after they have been exposed to IL-2 in the cell culture medium on Day 0 and/or Day 1. According to other embodiments, the cell culture medium comprises IL-2 during the first expansion and/or during the second expansion, and the gene-editing or transient cellular modification is carried out before the IL-2 is introduced into the cell culture medium. Alternatively, the cell culture medium may comprise IL-2 during the first expansion and/or during the second expansion, and the gene-editing or transient cellular modification is carried out after the IL-2 is introduced into the cell culture medium.

As discussed above, one or more of OKT-3, 4-1BB agonist and IL-2 may be included in the cell culture medium beginning on Day 0 or Day 1 of the first expansion. According to some embodiments, OKT-3 is included in the cell culture medium beginning on Day 0 or Day 1 of the first expansion, and/or a 4-1BB agonist is included in the cell culture medium beginning on Day 0 or Day 1 of the first expansion, and/or IL-2 is included in the cell culture medium beginning on Day 0 or Day 1 of the first expansion. According to other examples, the cell culture medium comprises OKT-3 and a 4-1BB agonist beginning on Day 0 or Day 1 of the first expansion. According to other examples, the cell culture medium comprises OKT-3, a 4-1BB agonist and IL-2 beginning on Day 0 or Day 1 of the first expansion. Of course, one or more of OKT-3, 4-1BB agonist and IL-2 may be added to the cell culture medium at one or more additional time points during the expansion process, as set forth in various embodiments described herein.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 for about 3 to 11 days to produce a second population of TILs, wherein the first expansion is performed in a closed container providing a first gas-permeable surface area;
(d) activating the second population of TILs by adding OKT-3 and culturing for about 1 to 2 days, wherein the transition from step (c) to step (d) occurs without opening the system;
(e) gene-editing at least a portion of the TIL cells in the second population of TILs to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells;
(f) optionally resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain a third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs; and
(i) transferring the harvested TIL population to an infusion bag,
   wherein the transfer from step (h) to (i) occurs without opening the system. In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, the TILs are rested after the gene-editing step and before the second expansion step. In some embodiments, the TILs are rested for about 1 to 2 days after the gene-editing step and before the second expansion step. In some embodiments, the TILs are activated by exposure to an anti-CD3 agonist and an anti-CD28 agonist for about 2 days. In some embodiments, the anti-CD3 agonist is an anti-CD3 agonist antibody and the anti-CD28 agonist is an anti-CD28 agonist antibody. In some embodiments, the anti-CD3 agonist antibody is OKT-3. In some embodiments, the TILs are activated by exposure to anti-CD3 agonist antibody- and anti-CD28 agonist antibody-conjugated beads. In some embodiments, the anti-CD3 agonist antibody- and anti-CD28 agonist antibody-conjugated beads are the TransAct™ product of Miltenyi. In some embodiments, the gene-editing process is carried out by viral transduction. In some embodiments, the gene-editing process is carried out by retroviral transduction of the TILs, optionally for about 2 days. In some embodiments, the gene-editing process is carried out by lentiviral transduction of the TILs, optionally for about 2 days. In some embodiments, the immunomodulatory composition is a membrane anchored immunomodulatory fusion protein. In some embodiments, the immunomodulatory fusion protein comprises IL-15. In some embodiments, the immunomodulatory fusion protein comprises IL-21. In some embodiments, the immunomodulatory composition comprises two or more different membrane bound fusion proteins. In some embodiments, the immunomodulatory composition comprises a first immunomodulatory protein comprising IL-15 and a second immunomodulatory fusion protein comprising IL-21. In some embodiments, the TILs are gene-edited to express the immunomodulatory composition under the control of an NFAT promoter. In some embodiments, the TILs are gene-edited to express an immunomodulatory fusion protein comprising IL-15 under the control of an NFAT promoter. In some embodiments, the TILs are gene-edited to express an immunomodulatory fusion protein comprising IL-21 under the control of an NFAT promoter. In some embodiments, the TILs are gene-edited to express a first immunomodulatory fusion protein comprising IL-15 and a second immunomodulatory fusion protein comprising IL-21 under the control of an NFAT promoter.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer of at least one gene editor into a portion of cells of the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain a third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs; and
(i) transferring the harvested TIL population to an infusion bag, wherein the transfer from step (h) to (i) occurs without opening the system,
   wherein the sterile electroporation of the at least one gene editor into the portion of cells of the second population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

According to other embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer of at least one nucleic acid molecule into a portion of cells of the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain a third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs; and
(i) transferring the harvested TIL population to an infusion bag, wherein the transfer from step (h) to (i) occurs without opening the system,
   wherein the sterile electroporation of the at least one nucleic acid molecule into the portion of cells of the second population of TILs modifies a plurality of cells in the portion to transiently express an immunomodulatory composition on the surface of the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer of at least one gene editor into a portion of cells of the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain a third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs; and
(i) transferring the harvested TIL population to an infusion bag, wherein the transfer from step (h) to (i) occurs without opening the system,
   wherein the sterile electroporation of the at least one gene editor into the portion of cells of the second population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) temporarily disrupting the cell membranes of the second population of TILs to effect transfer of at least one gene editor into a portion of cells of the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain a third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs; and
(i) transferring the harvested TIL population to an infusion bag, wherein the transfer from step (h) to (i) occurs without opening the system,
   wherein the at least one gene editor delivered into the portion of cells of the second population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

According to other embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) temporarily disrupting the cell membranes of the second population of TILs to effect transfer of at least one nucleic acid molecule into a portion of cells of the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain a third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs; and
(i) transferring the harvested TIL population to an infusion bag, wherein the transfer from step (h) to (i) occurs without opening the system,
   wherein the at least one nucleic acid molecule delivered into the portion of cells of the second population of TILs modifies a plurality of cells in the portion to transiently express an immunomodulatory composition on the surface of the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, a microfluidic platform is used to temporarily disrupt the cell membranes of the second population of TILs. In some embodiments, the microfluidic platform is a SQZ vector-free microfluidic platform.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) temporarily disrupting the cell membranes of the second population of TILs to effect transfer of at least one gene editor into a portion of cells of the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain a third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs; and
(i) transferring the harvested TIL population to an infusion bag, wherein the transfer from step (h) to (i) occurs without opening the system,
   wherein the at least one gene editor delivered into the portion of cells of the second population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) sterile electroporating the third population of TILs to effect transfer of at least one gene editor into a portion of cells of the third population of TILs to produce a fourth population of TILs; and
(e) culturing the fourth population of TILs in a second cell culture medium comprising antigen presenting cells (APCs), OKT-3, and IL-2 for about 5-15 days, to produce an expanded number of TILs,
   wherein the sterile electroporation of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the cells, and/or ii) an RNA molecule (e.g., an shRNA) for suppressing expression of an endogenous gene in the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing at least a portion of the TIL cells in the second population of TILs to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells; and
(e) culturing the fourth population of TILs in a second cell culture medium comprising antigen presenting cells (APCs), OKT-3, and IL-2 for about 5-15 days, to produce an expanded number of TILs.

In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, the TILs are rested after the gene-editing step and before the second expansion step. In some embodiments, the TILs are rested for about 1 to 2 days after the gene-editing step and before the second expansion step. In some embodiments, the TILs are activated by exposure to an anti-CD3 agonist and an anti-CD28 agonist for about 2 days. In some embodiments, the anti-CD3 agonist is an anti-CD3 agonist antibody and the anti-CD28 agonist is an anti-CD28 agonist antibody. In some embodiments, the anti-CD3 agonist antibody is OKT-3. In some embodiments, the TILs are activated by exposure to anti-CD3 agonist antibody- and anti-CD28 agonist antibody-conjugated beads. In some embodiments, the anti-CD3 agonist antibody- and anti-CD28 agonist antibody-conjugated beads are the TransAct™ product of Miltenyi. In some embodiments, the gene-editing process is carried out by viral transduction. In some embodiments, the gene-editing process is carried out by retroviral transduction of the TILs, optionally for about 2 days. In some embodiments, the gene-editing process is carried out by lentiviral transduction of the TILs, optionally for about 2 days. In some embodiments, the immunomodulatory composition is a membrane anchored immunomodulatory fusion protein. In some embodiments, the immunomodulatory fusion protein comprises IL-15. In some embodiments, the immunomodulatory fusion protein comprises IL-21. In some embodiments, the immunomodulatory composition comprises two or more different membrane bound fusion proteins. In some embodiments, the immunomodulatory composition comprises a first immunomodulatory protein comprising IL-15 and a second immunomodulatory fusion protein comprising IL-21. In some embodiments, the TILs are gene-edited to express the immunomodulatory composition under the control of an NFAT promoter. In some embodiments, the TILs are gene-edited to express an immunomodulatory fusion protein comprising IL-15 under the control of an NFAT promoter. In some embodiments, the TILs are gene-edited to express an immunomodulatory fusion protein comprising IL-21 under the control of an NFAT promoter. In some embodiments, the TILs are gene-edited to express a first immunomodulatory fusion protein comprising IL-15 and a second immunomodulatory fusion protein comprising IL-21 under the control of an NFAT promoter.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) sterile electroporating the third population of TILs to effect transfer of at least one nucleic acid molecule into a portion of cells of the third population of TILs to produce a fourth population of TILs; and
(e) culturing the fourth population of TILs in a second cell culture medium comprising antigen presenting cells (APCs), OKT-3, and IL-2 for about 5-15 days, to produce an expanded number of TILs,
   wherein the at least one nucleic acid molecule delivered into the portion of cells of the third population of TILs modifies a plurality of cells in the portion to transiently express an immunomodulatory composition on the surface of the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3-9 days to produce a second population of TILs;
(d) activating the second population of TILs using anti-CD3 and anti-CD28 beads or antibodies for 1-7 days, to produce a third population of TILs;
(e) gene-editing at least a portion of the TIL cells in the second population of TILs to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells; and
(f) culturing the fourth population of TILs in a second cell culture medium comprising antigen presenting cells (APCs), OKT-3, and IL-2 for about 5-15 days, to produce an expanded number of TILs.

In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, the TILs are rested after the gene-editing step and before the second expansion step. In some embodiments, the TILs are rested for about 1 to 2 days after the gene-editing step and before the second expansion step. In some embodiments, the TILs are activated by exposure to an anti-CD3 agonist and an anti-CD28 agonist for about 2 days. In some embodiments, the anti-CD3 agonist is an anti-CD3 agonist antibody and the anti-CD28 agonist is an anti-CD28 agonist antibody. In some embodiments, the anti-CD3 agonist antibody is OKT-3. In some embodiments, the TILs are activated by exposure to anti-CD3 agonist antibody- and anti-CD28 agonist antibody-conjugated beads. In some embodiments, the anti-CD3 agonist antibody- and anti-CD28 agonist antibody-conjugated beads are the TransAct™ product of Miltenyi. In some embodiments, the gene-editing process is carried out by viral transduction. In some embodiments, the gene-editing process is carried out by retroviral transduction of the TILs, optionally for about 2 days. In some embodiments, the gene-editing process is carried out by lentiviral transduction of the TILs, optionally for about 2 days. In some embodiments, the immunomodulatory composition is a membrane anchored immunomodulatory fusion protein. In some embodiments, the immunomodulatory fusion protein comprises IL-15. In some embodiments, the immunomodulatory fusion protein comprises IL-21. In some embodiments, the immunomodulatory composition comprises two or more different membrane bound fusion proteins. In some embodiments, the immunomodulatory composition comprises a first immunomodulatory protein comprising IL-15 and a second immunomodulatory fusion protein comprising IL-21. In some embodiments, the TILs are gene-edited to express the immunomodulatory composition under the control of an NFAT promoter. In some embodiments, the TILs are gene-edited to express an immunomodulatory fusion protein comprising IL-15 under the control of an NFAT promoter. In some embodiments, the TILs are gene-edited to express an immunomodulatory fusion protein comprising IL-21 under the control of an NFAT promoter. In some embodiments, the TILs are gene-edited to express a first immunomodulatory fusion protein comprising IL-15 and a second immunomodulatory fusion protein comprising IL-21 under the control of an NFAT promoter.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3-9 days to produce a second population of TILs;
(d) activating the second population of TILs using anti-CD3 and anti-CD28 beads or antibodies for 1-7 days, to produce a third population of TILs;
(e) sterile electroporating the third population of TILs to effect transfer of at least one gene editor into a portion of cells of the third population of TILs to produce a fourth population of TILs; and
(f) culturing the fourth population of TILs in a second cell culture medium comprising antigen presenting cells (APCs), OKT-3, and IL-2 for about 5-15 days, to produce an expanded number of TILs,
   wherein the sterile electroporation of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, the immunomodulatory agent is selected from the group consisting of IL-12, IL-15, IL-18, IL-21, and a CD40 agonist. In some embodiments

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3-9 days to produce a second population of TILs;
(d) activating the second population of TILs using anti-CD3 and anti-CD28 beads or antibodies for 1-7 days, to produce a third population of TILs;
(e) sterile electroporating the third population of TILs to effect transfer of at least one nucleic acid molecule into a portion of cells of the third population of TILs to produce a fourth population of TILs; and
(f) culturing the fourth population of TILs in a second cell culture medium comprising antigen presenting cells (APCs), OKT-3, and IL-2 for about 5-15 days, to produce an expanded number of TILs,
   wherein the at least one nucleic acid molecule delivered into the portion of cells of the third population of TILs modifies a plurality of cells in the portion to transiently express an immunomodulatory composition on the surface of the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) temporarily disrupting the cell membranes of the third population of TILs to effect transfer of at least one gene editor into a portion of cells of the third population of TILs to produce a fourth population of TILs; and
(e) culturing the fourth population of TILs in a second cell culture medium comprising antigen presenting cells (APCs), OKT-3, and IL-2 for about 5-15 days, to produce an expanded number of TILs,
   wherein the transfer of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) temporarily disrupting the cell membranes of the third population of TILs to effect transfer of at least one nucleic acid molecule into a portion of cells of the third population of TILs to produce a fourth population of TILs; and
(e) culturing the fourth population of TILs in a second cell culture medium comprising antigen presenting cells (APCs), OKT-3, and IL-2 for about 5-15 days, to produce an expanded number of TILs,
wherein the transfer of the at least one nucleic acid molecule into the portion of cells of the third population of TILs modifies a plurality of cells in the portion to transiently express an immunomodulatory composition on the surface of the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3-9 days to produce a second population of TILs;
(d) activating the second population of TILs using anti-CD3 and anti-CD28 beads or antibodies for 1-7 days, to produce a third population of TILs;
(e) temporarily disrupting the cell membranes of the third population of TILs to effect transfer of at least one gene editor into a portion of cells of the third population of TILs to produce a fourth population of TILs; and
(f) culturing the fourth population of TILs in a second cell culture medium comprising antigen presenting cells (APCs), OKT-3, and IL-2 for about 5-15 days, to produce an expanded number of TILs,
   wherein the transfer of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3-9 days to produce a second population of TILs;
(d) activating the second population of TILs using anti-CD3 and anti-CD28 beads or antibodies for 1-7 days, to produce a third population of TILs;
(e) temporarily disrupting the cell membranes of the third population of TILs to effect transfer of at least one nucleic acid molecule into a portion of cells of the third population of TILs to produce a fourth population of TILs; and
(f) culturing the fourth population of TILs in a second cell culture medium comprising antigen presenting cells (APCs), OKT-3, and IL-2 for about 5-15 days, to produce an expanded number of TILs,
wherein the transfer of the at least one nucleic acid molecule into the portion of cells of the third population of TILs modifies a plurality of cells in the portion to transiently express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, any of the foregoing methods is modified such that the step of culturing the fourth population of TILs is replaced with the steps of:
(f) culturing the fourth population of TILs in a second cell culture medium comprising antigen presenting cells (APCs), OKT-3, and IL-2 for about 1-7 days, to produce a culture of a fifth population of TILs; and
(g) splitting the culture of the fifth population of TILs into a plurality of subcultures, culturing each of the plurality of subcultures in a third cell culture medium comprising IL-2 for about 3-7 days, and combining the plurality of subcultures to provide an expanded number of TILs.

In some embodiments, the invention provides the method described in any of the preceding paragraphs as applicable above modified such that the step of activating the second population of TILs is performed for about 1 day, 2 days, 3 days, 4 days, 5 days, 6 days or 7 days.

In some embodiments, the invention provides the method described in any of the preceding paragraphs as applicable above modified such that the step of activating the second population of TILs is performed for about 2-7 days.

In some embodiments, the invention provides the method described in any of the preceding paragraphs as applicable above modified such that the step of activating the second population of TILs is performed for about 3-7 days.

In some embodiments, the invention provides the method described in any of the preceding paragraphs as applicable above modified such that the step of activating the second population of TILs is performed for about 4-7 days.

In some embodiments, the invention provides the method described in any of the preceding paragraphs as applicable above modified such that the step of activating the second population of TILs is performed for about 5-7 days.

In some embodiments, the invention provides the method described in any of the preceding paragraphs as applicable above modified such that the step of activating the second population of TILs is performed for about 6-7 days.

In some embodiments, the invention provides the method described in any of the preceding paragraphs as applicable above modified such that the step of activating the second population of TILs is performed for about 1-6 days.

In some embodiments, the invention provides the method described in any of the preceding paragraphs as applicable above modified such that the step of activating the second population of TILs is performed for about 1-5 days.

In some embodiments, the invention provides the method described in any of the preceding paragraphs as applicable above modified such that the step of activating the second population of TILs is performed for about 1-4 days.

In some embodiments, the invention provides the method described in any of the preceding paragraphs as applicable above modified such that the step of activating the second population of TILs is performed for about 1-3 days.

In some embodiments, the invention provides the method described in any of the preceding paragraphs as applicable above modified such that the step of activating the second population of TILs is performed for about 1-2 days.

In some embodiments, the invention provides the method described in any of the preceding paragraphs as applicable above modified such that the step of activating the second population of TILs is performed for about 2-6 days.

In some embodiments, the invention provides the method described in any of the preceding paragraphs as applicable above modified such that the step of activating the second population of TILs is performed for about 3-6 days.

In some embodiments, the invention provides the method described in any of the preceding paragraphs as applicable above modified such that the step of activating the second population of TILs is performed for about 4-6 days.

In some embodiments, the invention provides the method described in any of the preceding paragraphs as applicable above modified such that the step of activating the second population of TILs is performed for about 5-6 days.

In some embodiments, the invention provides the method described in any of the preceding paragraphs as applicable above modified such that the step of activating the second population of TILs is performed for about 3-5 days.

In some embodiments, the invention provides the method described in any of the preceding paragraphs as applicable above modified such that the step of activating the second population of TILs is performed for about 3-4 days.

In some embodiments, the invention provides the method described in any of the preceding paragraphs as applicable above modified such that the step of activating the second population of TILs is performed for about 2-5 days.

In some embodiments, the invention provides the method described in any of the preceding paragraphs as applicable above modified such that the step of activating the second population of TILs is performed for about 2-4 days.

In some embodiments, the invention provides the method described in any of the preceding paragraphs as applicable above modified such that the step of activating the second population of TILs is performed for about 2-3 days.

In some embodiments, the invention provides the method described in any of the preceding paragraphs as applicable above modified such that the step of activating the second population of TILs is performed for about 4-5 days.

In some embodiments, the invention provides the method described in any of the preceding paragraphs as applicable above modified such that the step of activating the second population of TILs is performed for about 1 day.

In some embodiments, the invention provides the method described in any of the preceding paragraphs as applicable above modified such that the step of activating the second population of TILs is performed for about 2 days.

In some embodiments, the invention provides the method described in any of the preceding paragraphs as applicable above modified such that the step of activating the second population of TILs is performed for about 3 days.

In some embodiments, the invention provides the method described in any of the preceding paragraphs as applicable above modified such that the step of activating the second population of TILs is performed for about 4 days.

In some embodiments, the invention provides the method described in any of the preceding paragraphs as applicable above modified such that the step of activating the second population of TILs is performed for about 5 days.

In some embodiments, the invention provides the method described in any of the preceding paragraphs as applicable above modified such that the step of activating the second population of TILs is performed for about 6 days.

In some embodiments, the invention provides the method described in any of the preceding paragraphs as applicable above modified such that the step of activating the second population of TILs is performed for about 7 days.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 and OKT-3 for about 3-9 days to produce a second population of TILs;
(c) sterile electroporating the second population of TILs to effect transfer of at least one gene editor into a portion of cells of the second population of TILs to produce a third population of TILs; and
(d) culturing the third population of TILs in a second cell culture medium comprising antigen presenting cells (APCs), OKT-3, and IL-2 for about 5-15 days, to produce an expanded number of TILs,
   wherein the sterile electroporation of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 and OKT-3 for about 3-9 days to produce a second population of TILs;
(c) sterile electroporating the second population of TILs to effect transfer of at least one nucleic acid molecule into a portion of cells of the second population of TILs to produce a third population of TILs; and
(d) culturing the third population of TILs in a second cell culture medium comprising antigen presenting cells (APCs), OKT-3, and IL-2 for about 5-15 days, to produce an expanded number of TILs,
   wherein the sterile electroporation of the at least one nucleic acid molecule into the portion of cells of the third population of TILs modifies a plurality of cells in the portion to transiently express an immunomodulatory composition on the surface of the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 and OKT-3 for about 3-9 days to produce a second population of TILs;
(d) sterile electroporating the second population of TILs to effect transfer of at least one gene editor into a portion of cells of the second population of TILs to produce a third population of TILs; and
(e) culturing the third population of TILs in a second cell culture medium comprising antigen presenting cells (APCs), OKT-3, and IL-2 for about 5-15 days, to produce an expanded number of TILs,
   wherein the sterile electroporation of the at least one gene editor into the portion of cells of the second population of TILs modifies a plurality of cells in the portion to express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an endogenous gene in the TIL cells. In some embodiments, the immunomodulatory composition comprises an immunomodulatory agent fused to a membrane anchor (e.g., a membrane anchored immunomodulatory fusion protein described herein). In some embodiments, the cytokine is selected from the group consisting of IL-2, IL-7, IL-10, IL-12, IL-15, IL-18, IL-21 and a CD40 agonist. In some embodiments, the cytokine is selected from the group consisting of IL-2, IL-12, IL-15, IL-18 and IL-21. In some embodiments, the cytokine is selected from the group consisting of IL-12, IL-15, IL-18, IL-21 and a CD40 agonist. In some embodiments, the endogenous gene encodes an immune checkpoint. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 and OKT-3 for about 3-9 days to produce a second population of TILs;
(d) sterile electroporating the second population of TILs to effect transfer of at least one nucleic acid molecule into a portion of cells of the second population of TILs to produce a third population of TILs; and
(e) culturing the third population of TILs in a second cell culture medium comprising antigen presenting cells (APCs), OKT-3, and IL-2 for about 5-15 days, to produce an expanded number of TILs,
   wherein the sterile electroporation of the at least one nucleic acid molecule into the portion of cells of the second population of TILs modifies a plurality of cells in the portion to transiently express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an immune checkpoint in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 and OKT-3 for about 3-9 days to produce a second population of TILs;
(c) temporarily disrupting the cell membranes of the second population of TILs to effect transfer of at least one gene editor into a portion of cells of the second population of TILs to produce a third population of TILs; and
(d) culturing the third population of TILs in a second cell culture medium comprising antigen presenting cells (APCs), OKT-3, and IL-2 for about 5-15 days, to produce an expanded number of TILs,
   wherein the transfer of the at least one gene editor into the portion of cells of the second population of TILs modifies a plurality of cells in the portion to express: i) an immunomodulatory composition comprising an immunomodulatory agent (e.g., a membrane anchored immunomodulatory fusion protein described herein) on the surface of the cells, and/or ii) an RNA molecule (e.g., an shRNA) for suppressing expression of an endogenous gene in the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 and OKT-3 for about 3-9 days to produce a second population of TILs;
(c) temporarily disrupting the cell membranes of the second population of TILs to effect transfer of at least one nucleic acid molecule into a portion of cells of the second population of TILs to produce a third population of TILs; and
(d) culturing the third population of TILs in a second cell culture medium comprising antigen presenting cells (APCs), OKT-3, and IL-2 for about 5-15 days, to produce an expanded number of TILs,
   wherein the transfer of the at least one gene editor into the portion of cells of the second population of TILs modifies a plurality of cells in the portion to transiently express an immunomodulatory composition on the surface of the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 and OKT-3 for about 3-9 days to produce a second population of TILs;
(d) temporarily disrupting the cell membranes of the second population of TILs to effect transfer of at least one gene editor into a portion of cells of the second population of TILs to produce a third population of TILs; and
(e) culturing the third population of TILs in a second cell culture medium comprising antigen presenting cells (APCs), OKT-3, and IL-2 for about 5-15 days, to produce an expanded number of TILs,
   wherein the transfer of the at least one gene editor into the portion of cells of the second population of TILs modifies a plurality of cells in the portion to express: i) an immunomodulatory composition comprising an immunomodulatory agent (e.g., a membrane anchored immunomodulatory fusion protein described herein) on the surface of the cells, and/or ii) an RNA molecule (e.g., an shRNA) for suppressing expression of an endogenous gene in the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 and OKT-3 for about 3-9 days to produce a second population of TILs;
(d) temporarily disrupting the cell membranes of the second population of TILs to effect transfer of at least one nucleic acid molecule into a portion of cells of the second population of TILs to produce a third population of TILs; and
(e) culturing the third population of TILs in a second cell culture medium comprising antigen presenting cells (APCs), OKT-3, and IL-2 for about 5-15 days, to produce an expanded number of TILs,
   wherein the transfer of the at least one nucleic acid molecule into the portion of cells of the second population of TILs modifies a plurality of cells in the portion to transiently express: i) an immunomodulatory composition comprising an immunomodulatory agent (e.g., a membrane anchored immunomodulatory fusion protein described herein) on the surface of the cells, and/or ii) an RNA molecule (e.g., an shRNA) for suppressing expression of an immune checkpoint in the cells. In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, the step of culturing the third population of TILs is performed by culturing the third population of TILs in the second cell culture medium for a first period of about 1-7 days, at the end of the first period the culture is split into a plurality of subcultures, each of the plurality of subcultures is cultured in a third culture medium comprising IL-2 for a second period of about 3-7 days, and at the end of the second period the plurality of subcultures are combined to provide the expanded number of TILs.

In some embodiments, the invention provides the method described in any of the preceding paragraphs as applicable above modified such that the step of culturing the first population of TILs in the first cell culture medium is performed for about 3 days, 4 days, 5 days, 6 days, 7 days, 8 days, 9 days, 10 days or 11 days.

In some embodiments, the invention provides the method described in any of the preceding paragraphs as applicable above modified such that the step of culturing the first population of TILs or the first expansion step is performed for about 4-11 days.

In some embodiments, the invention provides the method described in any of the preceding paragraphs as applicable above modified such that the step of culturing the first population of TILs or the first expansion step is performed for about 5-11 days.

In some embodiments, the invention provides the method described in any of the preceding paragraphs as applicable above modified such that the step of culturing the first population of TILs or the first expansion step is performed for about 6-11 days.

In some embodiments, the invention provides the method described in any of the preceding paragraphs as applicable above modified such that the step of culturing the first population of TILs or the first expansion step is performed for about 7-11 days.

In some embodiments, the invention provides the method described in any of the preceding paragraphs as applicable above modified such that the step of culturing the first population of TILs or the first expansion step is performed for about 8-11 days.

In some embodiments, the invention provides the method described in any of the preceding paragraphs as applicable above modified such that the step of culturing the first population of TILs or the first expansion step is performed for about 9-11 days.

In some embodiments, the invention provides the method described in any of the preceding paragraphs as applicable above modified such that the step of culturing the first population of TILs or the first expansion step is performed for about 10-11 days.

In some embodiments, the invention provides the method described in any of the preceding paragraphs as applicable above modified such that the step of culturing the first population of TILs or the first expansion step is performed for about 4-10 days.

In some embodiments, the invention provides the method described in any of the preceding paragraphs as applicable above modified such that the step of culturing the first population of TILs or the first expansion step is performed for about 5-10 days.

In some embodiments, the invention provides the method described in any of the preceding paragraphs as applicable above modified such that the step of culturing the first population of TILs or the first expansion step is performed for about 6-10 days.

In some embodiments, the invention provides the method described in any of the preceding paragraphs as applicable above modified such that the step of culturing the first population of TILs or the first expansion step is performed for about 7-10 days.

In some embodiments, the invention provides the method described in any of the preceding paragraphs as applicable above modified such that the step of culturing the first population of TILs or the first expansion step is performed for about 8-10 days.

In some embodiments, the invention provides the method described in any of the preceding paragraphs as applicable above modified such that the step of culturing the first population of TILs or the first expansion step is performed for about 9-10 days.

In some embodiments, the invention provides the method described in any of the preceding paragraphs as applicable above modified such that the step of culturing the first population of TILs in the first cell culture medium is performed for about 4-9 days.

In some embodiments, the invention provides the method described in any of the preceding paragraphs as applicable above modified such that the step of culturing the first population of TILs in the first cell culture medium is performed for about 5-9 days.

In some embodiments, the invention provides the method described in any of the preceding paragraphs as applicable above modified such that the step of culturing the first population of TILs in the first cell culture medium is performed for about 6-9 days.

In some embodiments, the invention provides the method described in any of the preceding paragraphs as applicable above modified such that the step of culturing the first population of TILs in the first cell culture medium is performed for about 7-9 days.

In some embodiments, the invention provides the method described in any of the preceding paragraphs as applicable above modified such that the step of culturing the first population of TILs in the first cell culture medium is performed for about 8-9 days.

In some embodiments, the invention provides the method described in any of the preceding paragraphs as applicable above modified such that the step of culturing the first population of TILs in the first cell culture medium is performed for about 3-8 days.

In some embodiments, the invention provides the method described in any of the preceding paragraphs as applicable above modified such that the step of culturing the first population of TILs in the first cell culture medium is performed for about 3-7 days.

In some embodiments, the invention provides the method described in any of the preceding paragraphs as applicable above modified such that the step of culturing the first population of TILs in the first cell culture medium is performed for about 3-6 days.

In some embodiments, the invention provides the method described in any of the preceding paragraphs as applicable above modified such that the step of culturing the first population of TILs in the first cell culture medium is performed for about 3-5 days.

In some embodiments, the invention provides the method described in any of the preceding paragraphs as applicable above modified such that the step of culturing the first population of TILs in the first cell culture medium is performed for about 3-4 days.

In some embodiments, the invention provides the method described in any of the preceding paragraphs as applicable above modified such that the step of culturing the first population of TILs in the first cell culture medium is performed for about 4-8 days.

In some embodiments, the invention provides the method described in any of the preceding paragraphs as applicable above modified such that the step of culturing the first population of TILs in the first cell culture medium is performed for about 4-7 days.

In some embodiments, the invention provides the method described in any of the preceding paragraphs as applicable above modified such that the step of culturing the first population of TILs in the first cell culture medium is performed for about 4-6 days.

In some embodiments, the invention provides the method described in any of the preceding paragraphs as applicable above modified such that the step of culturing the first population of TILs in the first cell culture medium is performed for about 4-6 days.

In some embodiments, the invention provides the method described in any of the preceding paragraphs as applicable above modified such that the step of culturing the first population of TILs in the first cell culture medium is performed for about 5-8 days.

In some embodiments, the invention provides the method described in any of the preceding paragraphs as applicable above modified such that the step of culturing the first population of TILs in the first cell culture medium is performed for about 5-7 days.

In some embodiments, the invention provides the method described in any of the preceding paragraphs as applicable above modified such that the step of culturing the first population of TILs in the first cell culture medium is performed for about 5-6 days.

In some embodiments, the invention provides the method described in any of the preceding paragraphs as applicable above modified such that the step of culturing the first population of TILs in the first cell culture medium is performed for about 6-8 days.

In some embodiments, the invention provides the method described in any of the preceding paragraphs as applicable above modified such that the step of culturing the first population of TILs in the first cell culture medium is performed for about 6-7 days.

In some embodiments, the invention provides the method described in any of the preceding paragraphs as applicable above modified such that the step of culturing the first population of TILs in the first cell culture medium is performed for about 7-8 days.

In some embodiments, the invention provides the method described in any of the preceding paragraphs as applicable above modified such that the step of culturing the first population of TILs in the first cell culture medium is performed for about 4-5 days.

In some embodiments, the invention provides the method described in any of the preceding paragraphs as applicable above modified such that the step of culturing the first population of TILs in the first cell culture medium is performed for about 3 days.

In some embodiments, the invention provides the method described in any of the preceding paragraphs as applicable above modified such that the step of culturing the first population of TILs in the first cell culture medium is performed for about 4 days.

In some embodiments, the invention provides the method described in any of the preceding paragraphs as applicable above modified such that the step of culturing the first population of TILs in the first cell culture medium is performed for about 5 days.

In some embodiments, the invention provides the method described in any of the preceding paragraphs as applicable above modified such that the step of culturing the first population of TILs in the first cell culture medium is performed for about 6 days.

In some embodiments, the invention provides the method described in any of the preceding paragraphs as applicable above modified such that the step of culturing the first population of TILs in the first cell culture medium is performed for about 7 days.

In some embodiments, the invention provides the method described in any of the preceding paragraphs as applicable above modified such that the step of culturing the first population of TILs in the first cell culture medium is performed for about 8 days.

In some embodiments, the invention provides the method described in any of the preceding paragraphs as applicable above modified such that the step of culturing the first population of TILs in the first cell culture medium is performed for about 9 days.

In some embodiments, the invention provides the method described in any of the preceding paragraphs as applicable above modified such that the step of culturing the first population of TILs in the first cell culture medium is performed for about 10 days.

In some embodiments, the invention provides the method described in any of the preceding paragraphs as applicable above modified such that the step of culturing the first population of TILs in the first cell culture medium is performed for about 11 days.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3 days to produce a second population of TILs;
(c) culturing the second population of TILs in a second cell culture medium comprising IL-2 and OKT-3 for 2-4 days to produce a third population of TILs;
(d)) sterile electroporating the third population of TILs to effect transfer of at least one gene editor into a portion of cells of the third population of TILs to produce a fourth population of TILs; and
(e) culturing the fourth population of TILs in a third cell culture medium comprising antigen presenting cells (APCs), OKT-3, and IL-2 for about 5-15 days, to produce an expanded number of TILs,
   wherein the sterile electroporation of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the cells, and/or ii) an RNA molecule (e.g., an shRNA) for suppressing expression of an endogenous gene in the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof. In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3 days to produce a second population of TILs;
(c) culturing the second population of TILs in a second cell culture medium comprising IL-2 and OKT-3 for 2-4 days to produce a third population of TILs;
(d)) sterile electroporating the third population of TILs to effect transfer of at least one nucleic acid molecule into a portion of cells of the third population of TILs to produce a fourth population of TILs; and
(e) culturing the fourth population of TILs in a third cell culture medium comprising antigen presenting cells (APCs), OKT-3, and IL-2 for about 5-15 days, to produce an expanded number of TILs,
   wherein the sterile electroporation of the at least one nucleic acid molecule into the portion of cells of the third population of TILs modifies a plurality of cells in the portion to transiently express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an immune checkpoint in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3 days to produce a second population of TILs;
(d) culturing the second population of TILs in a second cell culture medium comprising IL-2 and OKT-3 for 2-4 days to produce a third population of TILs;
(e) sterile electroporating the third population of TILs to effect transfer of at least one gene editor into a portion of cells of the third population of TILs to produce a fourth population of TILs; and
(f) culturing the fourth population of TILs in a third cell culture medium comprising antigen presenting cells (APCs), OKT-3, and IL-2 for about 5-15 days, to produce an expanded number of TILs,
   wherein the sterile electroporation of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the cells, and/or ii) an RNA molecule (e.g., an shRNA) for suppressing expression of an endogenous gene in the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3 days to produce a second population of TILs;
(d) culturing the second population of TILs in a second cell culture medium comprising IL-2 and OKT-3 for 2-4 days to produce a third population of TILs;
(e) sterile electroporating the third population of TILs to effect transfer of at least one nucleic acid molecule into a portion of cells of the third population of TILs to produce a fourth population of TILs; and
(f) culturing the fourth population of TILs in a third cell culture medium comprising antigen presenting cells (APCs), OKT-3, and IL-2 for about 5-15 days, to produce an expanded number of TILs,
   wherein the sterile electroporation of the at least one nucleic acid molecule into the portion of cells of the third population of TILs modifies a plurality of cells in the portion to transiently express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an immune checkpoint in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3 days to produce a second population of TILs;
(c) culturing the second population of TILs in a second cell culture medium comprising IL-2 and OKT-3 for 2-4 days to produce a third population of TILs;
(d)) temporarily disrupting the cell membranes of the third population of TILs to effect transfer of at least one gene editor into a portion of cells of the third population of TILs to produce a fourth population of TILs; and
(e) culturing the fourth population of TILs in a third cell culture medium comprising antigen presenting cells (APCs), OKT-3, and IL-2 for about 5-15 days, to produce an expanded number of TILs,
   wherein the transfer of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the cells, and/or ii) an RNA molecule (e.g., an shRNA) for suppressing expression of an endogenous gene in the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3 days to produce a second population of TILs;
(c) culturing the second population of TILs in a second cell culture medium comprising IL-2 and OKT-3 for 2-4 days to produce a third population of TILs;
(d)) temporarily disrupting the cell membranes of the third population of TILs to effect transfer of at least one nucleic acid molecule into a portion of cells of the third population of TILs to produce a fourth population of TILs; and
(e) culturing the fourth population of TILs in a third cell culture medium comprising antigen presenting cells (APCs), OKT-3, and IL-2 for about 5-15 days, to produce an expanded number of TILs,
   wherein the transfer of the at least one nucleic acid molecule into the portion of cells of the third population of TILs modifies a plurality of cells in the portion to transiently express an immunomodulatory composition on the surface of the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3 days to produce a second population of TILs;
(d) culturing the second population of TILs in a second cell culture medium comprising IL-2 and OKT-3 for 2-4 days to produce a third population of TILs;
(e) temporarily disrupting the cell membranes of the third population of TILs to effect transfer of at least one gene editor into a portion of cells of the third population of TILs to produce a fourth population of TILs; and
(f) culturing the fourth population of TILs in a third cell culture medium comprising antigen presenting cells (APCs), OKT-3, and IL-2 for about 5-15 days, to produce an expanded number of TILs,
   wherein the transfer of the at least one gene editor into the portion of cells of the third population of TILs modifies a plurality of cells in the portion to express: i) an immunomodulatory composition comprising an immunomodulatory agent (e.g., a membrane anchored immunomodulatory fusion protein described herein) on the surface of the cells, and/or ii) an RNA molecule (e.g., an shRNA) for suppressing expression of an endogenous gene in the cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3 days to produce a second population of TILs;
(d) culturing the second population of TILs in a second cell culture medium comprising IL-2 and OKT-3 for 2-4 days to produce a third population of TILs;
(e) temporarily disrupting the cell membranes of the third population of TILs to effect transfer of at least one nucleic acid molecule into a portion of cells of the third population of TILs to produce a fourth population of TILs; and
(f) culturing the fourth population of TILs in a third cell culture medium comprising antigen presenting cells (APCs), OKT-3, and IL-2 for about 5-15 days, to produce an expanded number of TILs,
   wherein the transfer of the at least one nucleic acid molecule into the portion of cells of the third population of TILs modifies a plurality of cells in the portion to transiently express: i) an immunomodulatory composition comprising an immunomodulatory agent (e.g., a membrane anchored immunomodulatory fusion protein described herein) on the surface of the TIL cells, and/or ii) an RNA molecule (e.g., an shRNA) for suppressing expression of an immune checkpoint in the TIL cells. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21 and a CD40 agonist (e.g., CD40L or an agonistic CD40 binding domain). In some embodiments, the immunomodulatory agent is selected from the group consisting of IL-2, IL-12, IL-15, IL-18, IL-21 and a CD40 agonist. In some embodiments, the immunomodulatory agent is selected from the group consisting of IL-12, IL-15, IL-18, IL-21, and a CD40 agonist. In some embodiments, the immunomodulatory agent is selected from the group consisting of IL-12, IL-15, IL-18, IL-21, and a CD40 agonist. In some embodiments, the immune checkpoint is selected from PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the endogenous gene is selected from PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, a microfluidic platform is used to temporarily disrupt the cell membranes of the second population of TILs. In some embodiments, the microfluidic platform is a SQZ vector-free microfluidic platform.

In some embodiments, the step of culturing the fourth population of TILs is performed by culturing the fourth population of TILs in the third cell culture medium for a first period of about 1-7 days, at the end of the first period the culture is split into a plurality of subcultures, each of the plurality of subcultures is cultured in a fourth culture medium comprising IL-2 for a second period of about 3-7 days, and at the end of the second period the plurality of subcultures are combined to provide the expanded number of TILs.

In some embodiments, in the step of culturing the first population of TILs in the first culture medium the first culture medium further comprises anti-CD3 and anti-CD28 beads or antibodies.

In some embodiments, the anti-CD3 and anti-CD28 beads or antibodies comprise the OKT-3 in the first culture medium.

In some embodiments, in the step of culturing the second population of TILs in the second culture medium the second culture medium further comprises anti-CD3 and anti-CD28 beads or antibodies.

In some embodiments, the anti-CD3 and anti-CD28 beads or antibodies comprise the OKT-3 in the second culture medium.

According to some embodiments, the foregoing method further comprises cryopreserving the harvested TIL population using a cryopreservation medium. In some embodiments, the cryopreservation medium is a dimethylsulfoxide-based cryopreservation medium. In other embodiments, the cryopreservation medium is CS10.

In some embodiments, the invention provides the method described in any preceding paragraph above modified as applicable such that the step of culturing the second population of TILs in the second culture medium is performed for about 2-3 days.

In some embodiments, the invention provides the method described in any preceding paragraph above modified as applicable such that the step of culturing the second population of TILs in the second culture medium is performed for about 3-4 days.

In some embodiments, the invention provides the method described in any preceding paragraph above modified as applicable such that the step of culturing the second population of TILs in the second culture medium is performed for about 2 days.

In some embodiments, the invention provides the method described in any preceding paragraph above modified as applicable such that the step of culturing the second population of TILs in the second culture medium is performed for about 3 days.

In some embodiments, the invention provides the method described in any of the preceding paragraphs as applicable above modified such that the step of culturing the second population of TILs in the second culture medium is performed for about 4 days.

In some embodiments, the invention provides the method described in any of the preceding paragraphs as applicable above modified such that the step of culturing the third or fourth population of TILs, as applicable, in the second or third cell culture medium, applicable, is performed for about 5 days, 6 days, 7 days, 8 days, 9 days, 10 days, 11 days, 12 days, 13 days, 14 days or 15 days.

In some embodiments, the invention provides the method described in any of the preceding paragraphs as applicable above modified such that the step of culturing the third or fourth population of TILs in the second or third cell culture medium is performed for about 6-15 days.

In some embodiments, the invention provides the method described in any of the preceding paragraphs as applicable above modified such that the step of culturing the third or fourth population of TILs in the second or third cell culture medium is performed for about 7-15 days.

In some embodiments, the invention provides the method described in any of the preceding paragraphs as applicable above modified such that the step of culturing the third or fourth population of TILs in the second or third cell culture medium is performed for about 8-15 days.

In some embodiments, the invention provides the method described in any of the preceding paragraphs as applicable above modified such that the step of culturing the third or fourth population of TILs in the second or third cell culture medium is performed for about 9-15 days.

In some embodiments, the invention provides the method described in any of the preceding paragraphs as applicable above modified such that the step of culturing the third or fourth population of TILs in the second or third cell culture medium is performed for about 10-15 days.

In some embodiments, the invention provides the method described in any of the preceding paragraphs as applicable above modified such that the step of culturing the third or fourth population of TILs in the second or third cell culture medium is performed for about 11-15 days.

In some embodiments, the invention provides the method described in any of the preceding paragraphs as applicable above modified such that the step of culturing the third or fourth population of TILs in the second or third cell culture medium is performed for about 12-15 days.

In some embodiments, the invention provides the method described in any of the preceding paragraphs as applicable above modified such that the step of culturing the third or fourth population of TILs in the second or third cell culture medium is performed for about 13-15 days.

In some embodiments, the invention provides the method described in any of the preceding paragraphs as applicable above modified such that the step of culturing the third or fourth population of TILs in the second or third cell culture medium is performed for about 14-15 days.

In some embodiments, the invention provides the method described in any of the preceding paragraphs as applicable above modified such that the step of culturing the third or fourth population of TILs in the second or third cell culture medium is performed for about 5-14 days.

In some embodiments, the invention provides the method described in any of the preceding paragraphs as applicable above modified such that the step of culturing the third or fourth population of TILs in the second or third cell culture medium is performed for about 6-14 days.

In some embodiments, the invention provides the method described in any of the preceding paragraphs as applicable above modified such that the step of culturing the third or fourth population of TILs in the second or third cell culture medium is performed for about 7-14 days.

In some embodiments, the invention provides the method described in any of the preceding paragraphs as applicable above modified such that the step of culturing the third or fourth population of TILs in the second or third cell culture medium is performed for about 8-14 days.

In some embodiments, the invention provides the method described in any of the preceding paragraphs as applicable above modified such that the step of culturing the third or fourth population of TILs in the second or third cell culture medium is performed for about 9-14 days.

In some embodiments, the invention provides the method described in any of the preceding paragraphs as applicable above modified such that the step of culturing the third or fourth population of TILs in the second or third cell culture medium is performed for about 10-14 days.

In some embodiments, the invention provides the method described in any of the preceding paragraphs as applicable above modified such that the step of culturing the third or fourth population of TILs in the second or third cell culture medium is performed for about 11-14 days.

In some embodiments, the invention provides the method described in any of the preceding paragraphs as applicable above modified such that the step of culturing the third or fourth population of TILs in the second or third cell culture medium is performed for about 12-14 days.

In some embodiments, the invention provides the method described in any of the preceding paragraphs as applicable above modified such that the step of culturing the third or fourth population of TILs in the second or third cell culture medium is performed for about 13-14 days.

In some embodiments, the invention provides the method described in any of the preceding paragraphs as applicable above modified such that the step of culturing the third or fourth population of TILs in the second or third cell culture medium is performed for about 5-13 days.

In some embodiments, the invention provides the method described in any of the preceding paragraphs as applicable above modified such that the step of culturing the third or fourth population of TILs in the second or third cell culture medium is performed for about 5-12 days.

In some embodiments, the invention provides the method described in any of the preceding paragraphs as applicable above modified such that the step of culturing the third or fourth population of TILs in the second or third cell culture medium is performed for about 5-11 days.

In some embodiments, the invention provides the method described in any of the preceding paragraphs as applicable above modified such that the step of culturing the third or fourth population of TILs in the second or third cell culture medium is performed for about 5-10 days.

In some embodiments, the invention provides the method described in any of the preceding paragraphs as applicable above modified such that the step of culturing the third or fourth population of TILs in the second or third cell culture medium is performed for about 5-9 days.

In some embodiments, the invention provides the method described in any of the preceding paragraphs as applicable above modified such that the step of culturing the third or fourth population of TILs in the second or third cell culture medium is performed for about 5-8 days.

In some embodiments, the invention provides the method described in any of the preceding paragraphs as applicable above modified such that the step of culturing the third or fourth population of TILs in the second or third cell culture medium is performed for about 5-7 days.

In some embodiments, the invention provides the method described in any of the preceding paragraphs as applicable above modified such that the step of culturing the third or fourth population of TILs in the second or third cell culture medium is performed for about 5-6 days.

In some embodiments, the invention provides the method described in any of the preceding paragraphs as applicable above modified such that the step of culturing the third or fourth population of TILs in the second or third cell culture medium is performed for about 6-13 days.

In some embodiments, the invention provides the method described in any of the preceding paragraphs as applicable above modified such that the step of culturing the third or fourth population of TILs in the second or third cell culture medium is performed for about 6-12 days.

In some embodiments, the invention provides the method described in any of the preceding paragraphs as applicable above modified such that the step of culturing the third or fourth population of TILs in the second or third cell culture medium is performed for about 6-11 days.

In some embodiments, the invention provides the method described in any of the preceding paragraphs as applicable above modified such that the step of culturing the third or fourth population of TILs in the second or third cell culture medium is performed for about 6-10 days.

In some embodiments, the invention provides the method described in any of the preceding paragraphs as applicable above modified such that the step of culturing the third or fourth population of TILs in the second or third cell culture medium is performed for about 6-9 days.

In some embodiments, the invention provides the method described in any of the preceding paragraphs as applicable above modified such that the step of culturing the third or fourth population of TILs in the second or third cell culture medium is performed for about 6-8 days.

In some embodiments, the invention provides the method described in any of the preceding paragraphs as applicable above modified such that the step of culturing the third or fourth population of TILs in the second or third cell culture medium is performed for about 6-7 days.

In some embodiments, the invention provides the method described in any of the preceding paragraphs as applicable above modified such that the step of culturing the third or fourth population of TILs in the second or third cell culture medium is performed for about 7-13 days.

In some embodiments, the invention provides the method described in any of the preceding paragraphs as applicable above modified such that the step of culturing the third or fourth population of TILs in the second or third cell culture medium is performed for about 7-12 days.

In some embodiments, the invention provides the method described in any of the preceding paragraphs as applicable above modified such that the step of culturing the third or fourth population of TILs in the second or third cell culture medium is performed for about 7-11 days.

In some embodiments, the invention provides the method described in any of the preceding paragraphs as applicable above modified such that the step of culturing the third or fourth population of TILs in the second or third cell culture medium is performed for about 7-10 days.

In some embodiments, the invention provides the method described in any of the preceding paragraphs as applicable above modified such that the step of culturing the third or fourth population of TILs in the second or third cell culture medium is performed for about 7-9 days.

In some embodiments, the invention provides the method described in any of the preceding paragraphs as applicable above modified such that the step of culturing the third or fourth population of TILs in the second or third cell culture medium is performed for about 7-8 days.

In some embodiments, the invention provides the method described in any of the preceding paragraphs as applicable above modified such that the step of culturing the third or fourth population of TILs in the second or third cell culture medium is performed for about 8-13 days.

In some embodiments, the invention provides the method described in any of the preceding paragraphs as applicable above modified such that the step of culturing the third or fourth population of TILs in the second or third cell culture medium is performed for about 8-12 days.

In some embodiments, the invention provides the method described in any of the preceding paragraphs as applicable above modified such that the step of culturing the third or fourth population of TILs in the second or third cell culture medium is performed for about 8-11 days.

In some embodiments, the invention provides the method described in any of the preceding paragraphs as applicable above modified such that the step of culturing the third or fourth population of TILs in the second or third cell culture medium is performed for about 8-10 days.

In some embodiments, the invention provides the method described in any of the preceding paragraphs as applicable above modified such that the step of culturing the third or fourth population of TILs in the second or third cell culture medium is performed for about 8-9 days.

In some embodiments, the invention provides the method described in any of the preceding paragraphs as applicable above modified such that the step of culturing the third or fourth population of TILs in the second or third cell culture medium is performed for about 9-13 days.

In some embodiments, the invention provides the method described in any of the preceding paragraphs as applicable above modified such that the step of culturing the third or fourth population of TILs in the second or third cell culture medium is performed for about 9-12 days.

In some embodiments, the invention provides the method described in any of the preceding paragraphs as applicable above modified such that the step of culturing the third or fourth population of TILs in the second or third cell culture medium is performed for about 9-11 days.

In some embodiments, the invention provides the method described in any of the preceding paragraphs as applicable above modified such that the step of culturing the third or fourth population of TILs in the second or third cell culture medium is performed for about 9-10 days.

In some embodiments, the invention provides the method described in any of the preceding paragraphs as applicable above modified such that the step of culturing the third or fourth population of TILs in the second or third cell culture medium is performed for about 10-13 days.

In some embodiments, the invention provides the method described in any of the preceding paragraphs as applicable above modified such that the step of culturing the third or fourth population of TILs in the second or third cell culture medium is performed for about 10-12 days.

In some embodiments, the invention provides the method described in any of the preceding paragraphs as applicable above modified such that the step of culturing the third or fourth population of TILs in the second or third cell culture medium is performed for about 10-11 days.

In some embodiments, the invention provides the method described in any of the preceding paragraphs as applicable above modified such that the step of culturing the third or fourth population of TILs in the second or third cell culture medium is performed for about 11-13 days.

In some embodiments, the invention provides the method described in any of the preceding paragraphs as applicable above modified such that the step of culturing the third or fourth population of TILs in the second or third cell culture medium is performed for about 11-12 days.

In some embodiments, the invention provides the method described in any of the preceding paragraphs as applicable above modified such that the step of culturing the third or fourth population of TILs in the second or third cell culture medium is performed for about 12-13 days.

In some embodiments, the invention provides the method described in any of the preceding paragraphs as applicable above modified such that the step of culturing the third or fourth population of TILs in the second or third cell culture medium is performed for about 5 days.

In some embodiments, the invention provides the method described in any of the preceding paragraphs as applicable above modified such that the step of culturing the third or fourth population of TILs in the second or third cell culture medium is performed for about 6 days.

In some embodiments, the invention provides the method described in any of the preceding paragraphs as applicable above modified such that the step of culturing the third or fourth population of TILs in the second or third cell culture medium is performed for about 7 days.

In some embodiments, the invention provides the method described in any of the preceding paragraphs as applicable above modified such that the step of culturing the third or fourth population of TILs in the second or third cell culture medium is performed for about 8 days.

In some embodiments, the invention provides the method described in any of the preceding paragraphs as applicable above modified such that the step of culturing the third or fourth population of TILs in the second or third cell culture medium is performed for about 9 days.

In some embodiments, the invention provides the method described in any of the preceding paragraphs as applicable above modified such that the step of culturing the third or fourth population of TILs in the second or third cell culture medium is performed for about 10 days.

In some embodiments, the invention provides the method described in any of the preceding paragraphs as applicable above modified such that the step of culturing the third or fourth population of TILs in the second or third cell culture medium is performed for about 11 days.

In some embodiments, the invention provides the method described in any of the preceding paragraphs as applicable above modified such that the step of culturing the third or fourth population of TILs in the second or third cell culture medium is performed for about 12 days.

In some embodiments, the invention provides the method described in any of the preceding paragraphs as applicable above modified such that the step of culturing the third or fourth population of TILs in the second or third cell culture medium is performed for about 13 days.

In some embodiments, the invention provides the method described in any of the preceding paragraphs as applicable above modified such that the step of culturing the third or fourth population of TILs in the second or third cell culture medium is performed for about 14 days.

In some embodiments, the invention provides the method described in any of the preceding paragraphs as applicable above modified such that the step of culturing the third or fourth population of TILs in the second or third cell culture medium is performed for about 15 days.

According to some embodiments, any of the foregoing methods may be used to provide an autologous harvested TIL population for the treatment of a human subject with cancer.

### C. Transient Cellular Modification

In some embodiments, the expanded TILs of the present invention are further manipulated before, during, or after an expansion step, including during closed, sterile manufacturing processes, each as provided herein, in order to alter protein expression in a transient manner. In some embodiments, the present invention includes transient cellular modification through nucleotide insertion, such as through ribonucleic acid (RNA) insertion, including insertion of messenger RNA (mRNA), into a population of TILs for promotion of the expression of one or more proteins or inhibition of the expression of one or more proteins, as well as simultaneous combinations of both promotion of one set of proteins with inhibition of another set of proteins.

In some embodiments, the expanded TILs of the present invention undergo transient alteration of protein expression. In some embodiments, the transient alteration of protein expression occurs in the bulk TIL population prior to first expansion. In some embodiments, the transient alteration of protein expression occurs after the first expansion. In some embodiments, the transient alteration of protein expression occurs in the bulk TIL population prior to second expansion. In some embodiments, the transient alteration of protein expression occurs after the second expansion.

In some embodiments, the transient alteration of protein expression results in transient expression of an immunomodulatory composition. In some embodiments, the immunomodulatory composition is an immunomodulatory fusion protein. In some embodiments, the immunomodulatory fusion protein comprises a membrane anchor fused to an immunomodulatory agent. In some embodiments, the immunomodulatory agent is selected from the group consisting of: IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18 and IL-21. In some embodiments, the immunomodulatory agent is an interleukin selected from the group consisting of IL-2, IL-12, IL-15, IL-18, IL-21, and a CD40 agonist (e.g., a CD40L or an agonistic CD40 binding domain). In some embodiments, the immunomodulatory agent is an interleukin selected from the group consisting of IL-2, IL-12, IL-15, IL-18, IL-21, and a CD40 agonist (e.g., a CD40L or an agonistic CD40 binding domain).

As discussed herein, embodiments of the present invention provide tumor infiltrating lymphocytes (TILs) that have been transiently modified via transient alteration of protein expression to enhance their therapeutic effect. Embodiments of the present invention embrace transient modification through nucleotide insertion (e.g., RNA) into a population of TILs for expression of an immunomodulatory composition. Embodiments of the present invention also provide methods for expanding TILs into a therapeutic population, wherein the methods comprise transient modification of the TILs. There are several gene-editing technologies that may be used to transiently modify a population of TILs, which are suitable for use in accordance with the present invention.

In some embodiments, a method of transiently altering protein expression in a population of TILs includes contacting the TILs with nucleic acid (e.g., mRNA) encoding the immunomodulatory composition and then subjecting the cells to the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In some embodiments, the electroporation method is a sterile electroporation method. In some embodiments, the electroporation method is a pulsed electroporation method. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained.

In some embodiments, a method of transiently altering protein expression in population of TILs includes the step of calcium phosphate transfection. Calcium phosphate transfection methods (calcium phosphate nucleic acid precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of transiently altering protein expression in a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of transiently altering protein expression in a population of TILs includes the step of transfection using methods described in U.S. Pat. Nos. 5,766,902; 6,025,337; 6,410,517; 6,475,994; and 7,189,705; the disclosures of each of which are incorporated by reference herein. The TILs may be a first population, a second population and/or a third population of TILs as described herein.

In some embodiments, a SQZ vector-free microfluidic platform is used for transiently altering protein expression. See, e.g., International Patent Application Publication Nos. WO 2013/059343A1, WO 2017/008063A1, or WO 2017/123663A1, or U.S. Patent Application Publication Nos. US 2014/0287509A1, US 2018/0201889A1, or US 2018/0245089A1, all of which are incorporated by reference herein in their entireties, and particularly for disclosures of microfluidic platforms for nucleic acid delivery. In the SQZ platform, the cell membranes of the TILs for modification are temporarily disrupted by microfluidic constriction, thereby allowing the delivery of nucleic acids encoding the transiently expressed protein. The TILs may be a first population, a second population and/or a third population of TILs as described herein.

In some embodiments of the present invention, a transposon/transposase system is used for transiently altering protein expression. In some embodiments, the transposon/transposase system comprises a piggyBac transposons and transposases or piggyBac-like transposons and transposases; a sleeping beauty (SB) transposon and transposase; or a Helraiser transposon and transposase.

### D. Immune Checkpoints

According to particular embodiments of the present invention, a TIL population is gene-edited to express one or more immunomodulatory compositions at the cell surface of TIL cells in the TIL population and to genetically modify one or more immune checkpoint genes in the TIL population. Stated another way, in addition to modification of a TIL population to express one or more immunomodulatory compositions at the cell surface, a DNA sequence within the TIL that encodes one or more of the TIL's immune checkpoints is permanently modified, e.g., inserted, deleted or replaced, in the TIL's genome, or transiently modified. Immune checkpoints are molecules expressed by lymphocytes that regulate an immune response via inhibitory or stimulatory pathways. In the case of cancer, immune checkpoint pathways are often activated to inhibit the anti-tumor response, i.e., the expression of certain immune checkpoints by malignant cells inhibits the anti-tumor immunity and favors the growth of cancer cells. See, e.g., Marin-Acevedo et al., Journal of Hematology & Oncology (2018) 11:39. Thus, certain inhibitory checkpoint molecules serve as targets for immunotherapies of the present invention. According to particular embodiments, TILs are gene-edited to block or stimulate certain immune checkpoint pathways and thereby enhance the body's immunological activity against tumors.

As used herein, an immune checkpoint gene comprises a DNA sequence encoding an immune checkpoint molecule. According to particular embodiments of the present invention, gene-editing TILs during the TIL expansion method causes expression of one or more immune checkpoint genes to be silenced or reduced in at least a portion of the therapeutic population of TILs. For example, gene-editing may cause the expression of an inhibitory receptor, such as PD-1 or CTLA-4, to be silenced or reduced in order to enhance an immune reaction.

The most broadly studied checkpoints include programmed cell death receptor-1 (PD-1) and cytotoxic T lymphocyte-associated molecule-4 (CTLA-4), which are inhibitory receptors on immune cells that inhibit key effector functions (e.g., activation, proliferation, cytokine release, cytotoxicity, etc.) when they interact with an inhibitory ligand. Numerous checkpoint molecules, in addition to PD-1 and CTLA-4, have emerged as potential targets for immunotherapy, as discussed in more detail below.

Non-limiting examples of immune checkpoint genes that may be silenced or inhibited by permanently gene-editing TILs of the present invention include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, BAFF (BR3), CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, and BCOR. For example, immune checkpoint genes that may be silenced or inhibited in TILs of the present invention may be selected from the group comprising PD-1, CTLA-4, LAG-3, TIM-3, Cish, CBL-B, TIGIT, TET2, TGFβ, and PKA. BAFF (BR3) is described in Bloom, et al., J Immunother., 2018, in press. According to another example, immune checkpoint genes that may be silenced or inhibited in TILs of the present invention may be selected from the group comprising PD-1, LAG-3, TIM-3, CTLA-4, TIGIT, TET2, CISH, TGFβR2, PRA, CBLB, BAFF (BR3), and combinations thereof.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days, wherein the second expansion is performed in a closed container providing a second gas-permeable surface area, and wherein the transition from step (f) to step (g) occurs without opening the system;
(h) harvesting the third population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system selected from the group consisting of a Clustered Regularly Interspersed Short Palindromic Repeat (CRISPR) system, a Transcription Activator-Like Effector (TALE) system, a zinc finger system, a Cas-CLOVER system, or an shRNA system, wherein the at least one gene editor system effects expression of at least one immunomodulatory composition at the cell surface of and inhibits expression of a molecule selected from the group consisting of PD-1, LAG-3, TIM-3, CTLA-4, TIGIT, TET2, CISH, TGFβR2, PRA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.), in the plurality of cells of the second population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 1. PD-1

One of the most studied targets for the induction of checkpoint blockade is the programmed death receptor (PD1 or PD-1, also known as PDCD1), a member of the CD28 super family of T-cell regulators. Its ligands, PD-L1 and PD-L2, are expressed on a variety of tumor cells, including melanoma. The interaction of PD-1 with PD-L1 inhibits T-cell effector function, results in T-cell exhaustion in the setting of chronic stimulation, and induces T-cell apoptosis in the tumor microenvironment. PD1 may also play a role in tumor-specific escape from immune surveillance.

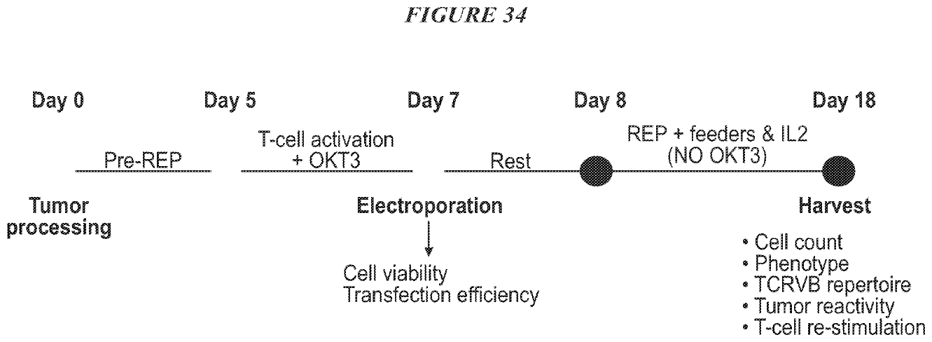

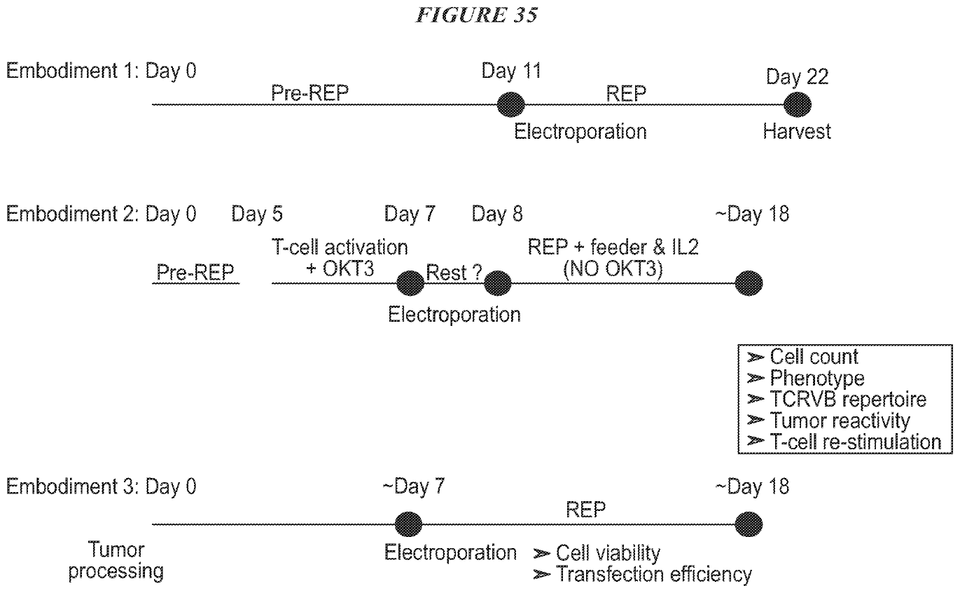

According to particular embodiments, expression of PD1 in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs by silencing or repressing the expression of PD1. As described in more detail below, the gene-editing process may involve the use of a programmable nuclease that mediates the generation of a double-strand or single-strand break at an immune checkpoint gene, such as PD1. For example, a CRISPR method, a TALE method, an RNA interference method (e.g., shRNA), a Cas-CLOVER method, or a zinc finger method may be used to silence or reduce the expression of PD1 in the TILs.

### 2. CTLA-4

CTLA-4 expression is induced upon T-cell activation on activated T-cells, and competes for binding with the antigen presenting cell activating antigens CD80 and CD86. Interaction of CTLA-4 with CD80 or CD86 causes T-cell inhibition and serves to maintain balance of the immune response. However, inhibition of the CTLA-4 interaction with CD80 or CD86 may prolong T-cell activation and thus increase the level of immune response to a cancer antigen.

According to particular embodiments, expression of CTLA-4 in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or repress the expression of CTLA-4 in the TILs. As described in more detail below, the gene-editing process may comprise the use of a programmable nuclease that mediates the generation of a double-strand or single-strand break at an immune checkpoint gene, such as CTLA-4. For example, a CRISPR method, a TALE method, an RNA interference method (e.g., shRNA), a Cas-CLOVER method, or a zinc finger method may be used to silence or repress the expression of CTLA-4 in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 3. LAG-3

Lymphocyte activation gene-3 (LAG-3, CD223) is expressed by T cells and natural killer (NK) cells after major histocompatibility complex (MHC) class II ligation. Although its mechanism remains unclear, its modulation causes a negative regulatory effect over T cell function, preventing tissue damage and autoimmunity. LAG-3 and PD-1 are frequently co-expressed and upregulated on TILs, leading to immune exhaustion and tumor growth. Thus, LAG-3 blockade improves anti-tumor responses. See, e.g., Marin-Acevedo et al., Journal of Hematology & Oncology (2018) 11:39.

According to particular embodiments, expression of LAG-3 in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or repress the expression of LAG-3 in the TILs. As described in more detail below, the gene-editing process may comprise the use of a programmable nuclease that mediates the generation of a double-strand or single-strand break at an immune checkpoint gene, such as LAG-3. According to particular embodiments, a CRISPR method, a TALE method, an RNA interference method (e.g., shRNA), a Cas-CLOVER method, or a zinc finger method may be used to silence or repress the expression of LAG-3 in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 4. TGIT

TGIT (T cell immunoreceptor with Ig and ITIM domains) is an immune receptor present on some T cells and natural killer (NK) cells. TGIT and PD-1 have been shown to be over-expressed on tumor antigen-specific CD8+ T cells and CD8+ TILs from individuals with melanoma. Blockage of TGIT and PD-1 has lead to increased cell proliferation, cytokine production, and degranulation of TA-specific CD8+ T cells and TIL CD8+ T cells.

According to particular embodiments, expression of TGIT in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or repress the expression of TGIT the TILs. As described in more detail below, the gene-editing process may comprise the use of a programmable nuclease that mediates the generation of a double-strand or single-strand break at an immune checkpoint gene, such as TGIT. According to particular embodiments, a CRISPR method, a TALE method, an RNA interference method (e.g., shRNA), a Cas-CLOVER method, or a zinc finger method may be used to silence or repress the expression of TGIT in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 5. TIM-3

T cell immunoglobulin-3 (TIM-3) is a direct negative regulator of T cells and is expressed on NK cells and macrophages. TIM-3 indirectly promotes immunosuppression by inducing expansion of myeloid-derived suppressor cells (MDSCs). Its levels have been found to be particularly elevated on dysfunctional and exhausted T-cells, suggesting an important role in malignancy.

According to particular embodiments, expression of TIM-3 in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or repress the expression of TIM-3 in the TILs. As described in more detail below, the gene-editing process may comprise the use of a programmable nuclease that mediates the generation of a double-strand or single-strand break at an immune checkpoint gene, such as TIM-3. For example, a CRISPR method, a TALE method, an RNA interference method (e.g., shRNA), a Cas-CLOVER method, or a zinc finger method may be used to silence or repress the expression of TIM-3 in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 6. Cish

Cish, a member of the suppressor of cytokine signaling (SOCS) family, is induced by TCR stimulation in CD8+ T cells and inhibits their functional avidity against tumors. Genetic deletion of Cish in CD8+ T cells may enhance their expansion, functional avidity, and cytokine polyfunctionality, resulting in pronounced and durable regression of established tumors. See, e.g., Palmer et al., Journal of Experimental Medicine, 212 (12): 2095 (2015).

According to particular embodiments, expression of Cish in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or repress the expression of Cish in the TILs. As described in more detail below, the gene-editing process may comprise the use of a programmable nuclease that mediates the generation of a double-strand or single-strand break at an immune checkpoint gene, such as Cish. For example, a CRISPR method, a TALE method, an RNA interference method (e.g., shRNA), a Cas-CLOVER method, or a zinc finger method may be used to silence or repress the expression of Cish in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 7. TGFβ

The TGFβ signaling pathway has multiple functions in regulating cell growth, differentiation, apoptosis, motility and invasion, extracellular matrix production, angiogenesis, and immune response. TGFβ signaling deregulation is frequent in tumors and has crucial roles in tumor initiation, development and metastasis. At the microenvironment level, the TGFβ pathway contributes to generate a favorable microenvironment for tumor growth and metastasis throughout carcinogenesis. See, e.g., Neuzillet et al., Pharmacology & Therapeutics, Vol. 147, pp. 22-31 (2015).

According to particular embodiments, expression of TGFβ in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or reduce the expression of TGFβ in the TILs. As described in more detail below, the gene-editing process may comprise the use of a programmable nuclease that mediates the generation of a double-strand or single-strand break at an immune checkpoint gene, such as TGFβ. For example, a CRISPR method, a TALE method, an RNA interference method (e.g., shRNA), a Cas-CLOVER method, or a zinc finger method may be used to silence or repress the expression of TGFβ in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

In some embodiments, TGFβR2 (TGF beta receptor 2) may be suppressed by silencing TGFβR2 using a CRISPR/Cas9 system or by using a TGFβR2 dominant negative extracellular trap, using methods known in the art.

### 8. PKA

Protein Kinase A (PKA) is a well-known member of the serine-threonine protein kinase superfamily. PKA, also known as cAMP-dependent protein kinase, is a multi-unit protein kinase that mediates signal transduction of G-protein coupled receptors through its activation upon cAMP binding. It is involved in the control of a wide variety of cellular processes from metabolism to ion channel activation, cell growth and differentiation, gene expression and apoptosis. Importantly, PKA has been implicated in the initiation and progression of many tumors. See, e.g., Sapio et al., EXCLI Journal; 2014; 13: 843-855.

According to particular embodiments, expression of PKA in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or repress the expression of PKA in the TILs. As described in more detail below, the gene-editing process may comprise the use of a programmable nuclease that mediates the generation of a double-strand or single-strand break at an immune checkpoint gene, such as PKA. For example, a CRISPR method, a TALE method, an RNA interference method (e.g., shRNA), a Cas-CLOVER method, or a zinc finger method may be used to silence or repress the expression of PKA in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 9. CBLB

CBLB (or CBL-B) is a E3 ubiquitin-protein ligase and is a negative regulator of T cell activation. Bachmaier, et al., Nature, 2000, 403, 211-216; Wallner, et al., Clin. Dev. Immunol. 2012, 692639.

According to particular embodiments, expression of CBLB in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silencing or repressing the expression of CBLB in TILs. As described in more detail below, the gene-editing process may comprise the use of a programmable nuclease that mediates the generation of a double-strand or single-strand break at an immune checkpoint gene, such as CBLB. For example, a CRISPR method, a TALE method, an RNA interference method (e.g., shRNA), a Cas-CLOVER method, or a zinc finger method may be used to silence or repress the expression of PKA in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain). In some embodiments, CBLB is silenced using a TALEN knockout. In some embodiments, CBLB is silenced using a TALE-KRAB transcriptional inhibitor knock in. More details on these methods can be found in Boettcher and McManus, Mol. Cell Review, 2015, 58, 575-585.

### 1. TIGIT

T-cell immunoreceptor with Ig and ITIM (immunoreceptor tyrosine-based inhibitory motif) domain or TIGIT is a transmembrane glycoprotein receptor with an Ig-like V-type domain and an ITIM in its cytoplasmic domain. Khalil, et al., Advances in Cancer Research, 2015, 128, 1-68; Yu, et al., Nature Immunology, 2009, Vol. 10, No. 1, 48-57. TIGIT is expressed by some T cells and Natural Killer Cells. Additionally, TIGIT has been shown to be overexpressed on antigen-specific CD8+ T cells and CD8+ TILs, particularly from individuals with melanoma. Studies have shown that the TIGIT pathway contributes to tumor immune evasion and TIGIT inhibition has been shown to increase T-cell activation and proliferation in response to polyclonal and antigen-specific stimulation. Khalil, et al., Advances in Cancer Research, 2015, 128, 1-68. Further, coblockade of TIGIT with either PD-1 or TIM3 has shown synergistic effects against solid tumors in mouse models. Id.; see also Kurtulus, et al., The Journal of Clinical Investigation, 2015, Vol. 125, No. 11, 4053-4062.

According to particular embodiments, expression of TIGIT in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or repress the expression of TIGIT in the TILs. As described in more detail below, the gene-editing process may comprise the use of a programmable nuclease that mediates the generation of a double-strand or single-strand break at an immune checkpoint gene, such as TIGIT. For example, a CRISPR method, a TALE method, an RNA interference method (e.g., shRNA), a Cas-CLOVER method, or a zinc finger method may be used to silence or repress the expression of TIGIT in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 10. TOX

Thymocyte selection associated high mobility group (HMG) box (TOX) is a transcription factor containing an HMG box DNA binding domain. TOX is a member of the HMG box superfamily that is thought to bind DNA in a sequence-independent but structure-dependent manner.

TOX was identified as a critical regulator of tumor-specific CD8⁺ T cell dysfunction or T cell exhaustion and was found to transcriptionally and epigenetically program CD8⁺ T cell exhaustion, as described, for example in Scott, et al., Nature, 2019, 571, 270-274 and Khan, et al., Nature, 2019, 571, 211-218, both of which are herein incorporated by reference in their entireties. TOX was also found to be critical factor for progression of T cell dysfunction and maintenance of exhausted T cells during chronic infection, as described in Alfei, et al., Nature, 2019, 571, 265-269, which is herein incorporated by reference in its entirety. TOX is highly expressed in dysfunctional or exhausted T cells from tumors and chronic viral infection. Ectopic expression of TOX in effector T cells in vitro induced a transcriptional program associated with T cell exhaustion, whereas deletion of TOX in T cells abrogated the T exhaustion program.

According to particular embodiments, expression of TOX in TILs is silenced or reduced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and silence or repress the expression of TOX. As described in more detail below, the gene-editing process may comprise the use of a programmable nuclease that mediates the generation of a double-strand or single-strand break at an immune checkpoint gene, such as TOX. For example, a CRISPR method, a TALE method, an RNA interference method (e.g., shRNA), a Cas-CLOVER method, or a zinc finger method may be used to silence or repress the expression of TOX in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### E. Expression/Overexpression of Co-Stimulatory Receptors or Adhesion Molecules

According to additional embodiments, gene-editing TILs during the TIL expansion method causes expression of at least one immunomodulatory composition at the cell surface and causes expression of one or more co-stimulatory receptors, adhesion molecules and/or cytokines to be enhanced in at least a portion of the therapeutic population of TILs. For example, gene-editing may cause the expression of a co-stimulatory receptor, adhesion molecule or cytokine to be enhanced, which means that it is overexpressed as compared to the expression of a co-stimulatory receptor, adhesion molecule or cytokine that has not been genetically modified. Non-limiting examples of co-stimulatory receptor, adhesion molecule or cytokine genes that may exhibit enhanced expression by permanently gene-editing TILs of the present invention include certain chemokine receptors and interleukins, such as CCR2, CCR4, CCR5, CXCR2, CXCR3, CX3CR1, IL-2, IL-4, IL-7, IL-10, IL-15, IL-18, IL-21, the NOTCH 1/2 intracellular domain (ICD), and/or the NOTCH ligand mDLL1.

### 1. CCRs

For adoptive T cell immunotherapy to be effective, T cells need to be trafficked properly into tumors by chemokines. A match between chemokines secreted by tumor cells, chemokines present in the periphery, and chemokine receptors expressed by T cells is important for successful trafficking of T cells into a tumor bed.

According to particular embodiments, gene-editing methods of the present invention may be used to increase the expression of certain chemokine receptors in the TILs, such as one or more of CCR2, CCR4, CCR5, CXCR2, CXCR3 and CX3CR1. Over-expression of CCRs may help promote effector function and proliferation of TILs following adoptive transfer.

According to particular embodiments, expression of one or more of CCR2, CCR4, CCR5, CXCR2, CXCR3 and CX3CR1 in TILs is enhanced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs to express at least one immunomodulatory composition at the cell surface of and enhance the expression of one or more of CCR2, CCR4, CCR5, CXCR2, CXCR3 and CX3CR1 in the TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

As described in more detail below, the gene-editing process may comprise the use of a programmable nuclease that mediates the generation of a double-strand or single-strand break at a chemokine receptor gene. For example, a CRISPR method, a TALE method, or a zinc finger method may be used to enhance the expression of certain chemokine receptors in the TILs.

In some embodiments, CCR4 and/or CCR5 adhesion molecules are inserted into a TIL population using a gamma-retroviral or lentiviral method as described herein. In some embodiments, CXCR2 adhesion molecule are inserted into a TIL population using a gamma-retroviral or lentiviral method as described in Forget, et al., Frontiers Immunology 2017, 8, 908 or Peng, et al., Clin. Cancer Res. 2010, 16, 5458, the disclosures of which are incorporated by reference herein.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, and wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11, wherein the second expansion is performed in a closed container providing a second gas-permeable surface area, and wherein the transition from step (f) to step (g) occurs without opening the system;
(h) harvesting the third population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system selected from the group consisting of a Clustered Regularly Interspersed Short Palindromic Repeat (CRISPR) system, a Transcription Activator-Like Effector (TALE) system, or a zinc finger system, wherein the at least one gene editor system effects expression of at least one immunomodulatory composition at the cell surface of and inhibits expression of PD-1 and, optionally, LAG-3, in the plurality of cells of the second population of TILs, and further wherein the at least one gene editor system effects expression of a CXCR2 adhesion molecule at the cell surface of the plurality of cells of the second population of TILs or the CXCR2 adhesion molecule is inserted by a gammaretroviral or lentiviral method into the first population of TILs, second population of TILs, or harvested population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, and wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days, wherein the second expansion is performed in a closed container providing a second gas-permeable surface area, and wherein the transition from step (f) to step (g) occurs without opening the system;
(h) harvesting the third population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system selected from the group consisting of a Clustered Regularly Interspersed Short Palindromic Repeat (CRISPR) system, a Transcription Activator-Like Effector (TALE) system, or a zinc finger system, which at least one gene editor system effects expression of at least one immunomodulatory composition at the cell surface of and inhibits expression of PD-1 and, optionally, LAG-3, in the plurality of cells of the second population of TILs and further wherein the at least one gene editor system effects expression of a CCR4 and/or CCR5 adhesion molecule at the cell surface of the plurality of cells of the second population of TILs or the CXCR2 adhesion molecule is inserted by a gammaretroviral or lentiviral method into the first population of TILs, second population of TILs, or harvested population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days, wherein the second expansion is performed in a closed container providing a second gas-permeable surface area, and wherein the transition from step (f) to step (g) occurs without opening the system;
(h) harvesting the third population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system selected from the group consisting of a Clustered Regularly Interspersed Short Palindromic Repeat (CRISPR) system, a Transcription Activator-Like Effector (TALE) system, or a zinc finger system, which at least one gene editor system effects expression of at least one immunomodulatory composition at the cell surface of and inhibits expression of PD-1 and, optionally, LAG-3, in the plurality of cells of the second population of TILs, and further wherein the at least one gene editor system effects expression of an adhesion molecule selected from the group consisting of CCR2, CCR4, CCR5, CXCR2, CXCR3, CX3CR1, and combinations thereof, at the cell surface of the plurality of cells of the second population of TILs or the adhesion molecule is inserted by a gammaretroviral or lentiviral method into the first population of TILs, second population of TILs, or harvested population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

### 2. Interleukins

According to additional embodiments, gene-editing methods of the present invention may be used to increase the expression of certain interleukins, such as one or more of IL-2, IL-4, IL-7, IL-10, IL-15, IL-18 and IL-21. Certain interleukins have been demonstrated to augment effector functions of T cells and mediate tumor control.

According to particular embodiments, expression of one or more of IL-2, IL-4, IL-7, IL-10, IL-15, IL-18 and IL-21 in TILs is enhanced in accordance with compositions and methods of the present invention. For example, a method for expanding tumor infiltrating lymphocytes (TILs) into a therapeutic population of TILs may be carried out in accordance with any embodiment of the methods described herein (e.g., process 2A, process Gen 3, or the methods shown in FIGS. 34 and 35), wherein the method comprises gene-editing at least a portion of the TILs by enhancing the expression of one or more of IL-2, IL-4, IL-7, IL-10, IL-15, IL-18 and IL-21. As described in more detail below, the gene-editing process may comprise the use of a programmable nuclease that mediates the generation of a double-strand or single-strand break at an interleukin gene. For example, a CRISPR method, a TALE method, or a zinc finger method may be used to enhance the expression of certain interleukins in the TILs.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) sterile electroporating the second population of TILs to effect transfer of at least one gene editor;
(f) resting the second population of TILs for about 1 day into a plurality of cells in the second population of TILs;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system selected from the group consisting of a Clustered Regularly Interspersed Short Palindromic Repeat (CRISPR) system, a Transcription Activator-Like Effector (TALE) system, or a zinc finger system, which at least one gene editor system effects expression of at least one immunomodulatory composition at the cell surface of and inhibits expression of PD-1 and, optionally, LAG-3, in the plurality of cells of the second population of TILs and further wherein the at least one gene editor system effects expression of an interleukin selected from the group consisting of IL-2, IL-4, IL-7, IL-10, IL-15, IL-18, IL-21, and combinations thereof, at the cell surface of the plurality of cells of the second population of TILs or the interleukin is inserted by a gammaretroviral or lentiviral method into the first population of TILs, second population of TILs, or harvested population of TILs. In some embodiments, the at least one immunomodulatory composition comprises a cytokine fused to a membrane anchor. In some embodiments, the cytokine is selected from the group consisting of IL-12, IL-15, IL-18 and IL-21.

### F. Gene Editing Methods

As discussed above, embodiments of the present invention provide tumor infiltrating lymphocytes (TILs) that have been genetically modified via gene-editing, including TILs that have been modified via transient gene-editing to transiently alter protein expression in the modified TILs, to enhance their therapeutic effect. Embodiments of the present invention embrace genetic editing through nucleotide insertion (RNA or DNA) into a population of TILs for both promotion of the expression of one or more proteins and inhibition of the expression of one or more proteins, as well as combinations thereof. Embodiments of the present invention also provide methods for expanding TILs into a therapeutic population, wherein the methods comprise gene-editing the TILs, or wherein the methods comprise transiently gene-editing the TILs to transiently alter protein expression in the modified TILs. There are several gene-editing technologies that may be used to genetically modify a population of TILs, including transient gene-editing technologies to transiently alter protein expression in a population of TILs, which are suitable for use in accordance with the present invention.

In some embodiments, a method of genetically modifying a population of TILs includes the step of stable incorporation of genes for production of one or more proteins or nucleic acid (e.g., an immunomodulatory fusion protein and/or an RNA (e.g., an shRNA) for modifying gene expression as described herein). In some embodiments, a method of genetically modifying a population of TILs includes the step of retroviral transduction. In some embodiments, a method of genetically modifying a population of TILs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein.

Lentiviral vector for genetically modify the subject TILs can be made using any suitable technique known in the art. In some embodiments, the lentiviral vector is made using a stable lentiviral producer cell line. In some embodiments, the stable lentiviral producer cell line is made using transposon/transposase (e.g., piggyBac and Sleeping Beauty systems described herein) to integrate various genes for lentiviral vector production (e.g., VSV-g/BaEV-TR, gag, and pol genes, gene of interest, etc.) into the producer cell line genome. In some embodiments, the genes for lentiviral vector production are under the control of inducible expression systems (e.g., Tet-on and Tet-off systems) that allow for inducible production of the lentiviral vectors. Exemplary inducible stable producer cell lines for lentiviral production, include for example, the EuLV® (Eureka Bio) system, see, e.g., Xue et al., Cell & Gene Therapy Insights 8(2):199-209 (2022), WO2021218000A1, WO2021232632A1, and WO2021232633A1, which are incorporated by reference in their entireties, particularly for pertinent parts relating to the production of inducible stable producer cell line for lentiviral production.

In some embodiments, a method of genetically modifying a population of TILs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In some embodiments, a method of genetically modifying a population of TILs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some embodiments, a method of genetically modifying a population of TILs includes the step of stable incorporation of genes for production or inhibition (e.g., silencing) of one or more proteins. In some embodiments, a method of genetically modifying, such as a method of transient genetic modification by transiently altering protein expression in, a population of TILs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In some embodiments, the electroporation method is a sterile electroporation method. In some embodiments, the electroporation method is a pulsed electroporation method. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained.

In some embodiments, a method of genetically modifying, such as a method of transient genetic modification by transiently altering protein expression in, a population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of genetically modifying, such as a method of transient genetic modification by transiently altering protein expression in, a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of genetically modifying, such as a method of transient genetic modification by transiently altering protein expression in, a population of TILs includes the step of transfection using methods described in U.S. Pat. Nos. 5,766,902; 6,025,337; 6,410,517; 6,475,994; and 7,189,705; the disclosures of each of which are incorporated by reference herein.

According to some embodiments, the gene-editing process may comprise the use of a programmable nuclease that mediates the generation of a double-strand or single-strand break at one or more immune checkpoint genes. Such programmable nucleases enable precise genome editing by introducing breaks at specific genomic loci, i.e., they rely on the recognition of a specific DNA sequence within the genome to target a nuclease domain to this location and mediate the generation of a double-strand break at the target sequence. A double-strand break in the DNA subsequently recruits endogenous repair machinery to the break site to mediate genome editing by either non-homologous end-joining (NHEJ) or homology-directed repair (HDR). Thus, the repair of the break can result in the introduction of insertion/deletion mutations that disrupt (e.g., silence, repress, or enhance) the target gene product.

Major classes of nucleases that have been developed to enable site-specific genomic editing include zinc finger nucleases (ZFNs), transcription activator-like nucleases (TALENs), and CRISPR-associated nucleases (e.g., CRISPR/Cas9). These nuclease systems can be broadly classified into two categories based on their mode of DNA recognition: ZFNs and TALENs achieve specific DNA binding via protein-DNA interactions, whereas CRISPR systems, such as Cas9, are targeted to specific DNA sequences by a short RNA guide molecule that base-pairs directly with the target DNA and by protein-DNA interactions. See, e.g., Cox et al., Nature Medicine, 2015, Vol. 21, No. 2.

Non-limiting examples of gene-editing methods that may be used in accordance with TIL expansion methods of the present invention include CRISPR methods, TALE methods, ZFN methods, Cas-CLOVER methods, shRNA methods, or a combination thereof, embodiments of which are described in more detail below. According to some embodiments, a method for expanding TILs into a therapeutic population may be carried out in accordance with any embodiment of the methods described herein (e.g., process 2A) or as described in PCT/US2017/058610, PCT/US2018/012605, or PCT/US2018/012633, wherein the method further comprises gene-editing at least a portion of the TILs by one or more of a CRISPR method, a TALE method, a ZFN method, a Cas-CLOVER method, or an shRNA method, in order to generate TILs that can provide an enhanced therapeutic effect. According to some embodiments, gene-edited TILs can be evaluated for an improved therapeutic effect by comparing them to non-modified TILs in vitro, e.g., by evaluating in vitro effector function, cytokine profiles, etc. compared to unmodified TILs.

In some embodiments of the present invention, electroporation is used for delivery of a gene editing system, such as CRISPR, TALEN, ZFN, Cas-CLOVER, and shRNA systems. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

In some embodiments, a microfluidic platform is used for delivery of the gene editing system. In some embodiments, the microfluidic platform is a SQZ vector-free microfluidic platform.

### Transposon/Transposase Delivery Methods

In some embodiments of the present invention, a transposon/transposase system is used for delivery of a gene editing system, such as CRISPR, TALEN, ZFN, Cas-CLOVER, and shRNA systems.

A method for expanding TILs into a therapeutic population may be carried out in accordance with any embodiment of the methods described herein (e.g., process 2A) or as described in PCT/US2017/058610, PCT/US2018/012605, or PCT/US2018/012633, wherein the method further comprises gene-editing at least a portion of the TILs by a transposon/transposase system (e.g., piggyBac transposons and transposases or piggyBac-like transposons and transposases; sleeping beauty (SB) transposon and transposase; Helraiser transposon and transposase, etc.).

According to particular embodiments, the use of a piggyBac, Sleeping Beauty or Helraiser method during the TIL expansion process causes expression of at least one immunomodulatory composition at the cell surface of at least a portion of the therapeutic population of TILs. Alternatively, the use of a piggyBac, Sleeping Beauty or Helraiser method during the TIL expansion process causes expression of at least one immunomodulatory composition at the cell surface of, and optionally causes one or more immune checkpoint genes to be enhanced in, at least a portion of the therapeutic population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, the use of a piggyBac, Sleeping Beauty or Helraiser method during the TIL expansion process causes reduced expression of a target gene in at least a portion of the therapeutic population of TILs. In some embodiments, the target gene is an immune checkpoint.

The piggyBac transposon is a mobile genetic element that efficiently transposes between the donor vector and host chromosomes. This system has almost no cargo limit, and is fully reversible, leaving no footprint in the genome after excision. The piggyBac transposon/transposase system consists of a transposase that recognizes piggyBac-specific inverted terminal repeat sequences (ITRs) located on both sides of the transposon cassette. The transposase excises the transposable element to integrate it into TT/AA chromosomal sites that are preferentially located in euchromatic regions of mammalian genomes (Ding et al. 2005; Cadinaños and Bradley 2007; Wilson et al. 2007; Wang et al. 2008; Li et al. 2011).

Exemplary piggyBac systems include those described in WO2019/046815, the contents of which are incorporated herein by reference in their entirety. In some embodiments, the piggyBac system comprises a transposon/transposase system.

In some embodiments, a piggyBac method comprises delivering to the TILs, (a) a nucleic acid or amino acid sequence comprising a sequence encoding a transposase enzyme and (b) a recombinant and non-naturally occurring DNA sequence comprising a DNA sequence encoding a transposon.

In some embodiments, the sequence encoding a transposase enzyme is an mRNA sequence. In some embodiments, the sequence encoding a transposase enzyme is a DNA sequence. In some embodiments, the DNA sequence is a cDNA sequence. In some embodiments, the sequence encoding a transposase enzyme is an amino acid sequence. A protein Super piggybac transposase (SPB) may be delivered following pre-incubation with transposon DNA.

### Transposons Transposases

Exemplary transposon/transposase systems include, but are not limited to, piggyBac transposons and transposases, Sleeping Beauty transposons and transposases, Helraiser transposons and transposases and Tol2 transposons and transposases.

The piggyBac transposase recognizes transposon-specific inverted terminal repeat sequences (ITRs) on the ends of the transposon, and moves the contents between the ITRs into TTAA chromosomal sites. The piggyBac transposon system has no payload limit for the genes of interest that can be included between the ITRs. In some embodiments, the transposon is a piggyBac transposon or a piggyBac-like transposon.

Examples of piggyBac and piggyBac-like transposases and transposons include, for example, those disclosed in WO2019/046815, the contents of which are incorporated herein by reference in their entirety. In some embodiments, the piggyBac or piggyBac-like transposase is hyperactive. A hyperactive piggyBac or piggyBac-like transposase is a transposase that is more active than the naturally occurring variant from which it is derived. In some embodiments, the hyperactive piggyBac or piggyBac-like transposase enzyme is isolated or derived from Bombyx mori. A list of hyperactive amino acid substitutions can be found in U.S. Pat. No. 10,041,077, the contents of which are incorporated herein by reference in their entirety. In some embodiments, the piggyBac or piggyBac-like transposase is integration deficient. In some embodiments, an integration deficient piggyBac or piggyBac-like transposase is a transposase that can excise its corresponding transposon, but that integrates the excised transposon at a lower frequency than a corresponding wild-type transposase. A list of integration deficient amino acid substitutions can be found in U.S. Pat. No. 10,041,077, the contents of which are incorporated by reference in their entirety.

In some embodiments, the piggyBac or piggyBac-like transposon is capable of insertion by a piggyBac or piggyBac-like transposase at the sequence 5′-TTAT-3 within a target nucleic acid. In some embodiments, and, in particular, embodiments wherein the transposon is a piggyBac transposon, the transposase is a piggyBac transposase. In some embodiments, and, in particular, embodiments wherein the transposon is a piggyBac-like transposon, the transposase is a piggyBac-like transposase. In some embodiments, and, in particular, embodiments wherein the transposon is a piggyBac transposon, the transposase is a piggyBac™ or a Super piggyBac™ (SPB) transposase. In some embodiments, and, in particular, embodiments wherein the transposase is a Super piggyBac™ (SPB) transposase, the sequence encoding the transposase is an mRNA sequence.

The sleeping beauty (SB) transposon is transposed into the target genome by the Sleeping Beauty transposase that recognizes ITRs, and moves the contents between the ITRs into TA chromosomal sites. In some embodiments, the transposon is a Sleeping Beauty transposon. In some embodiments, the transposase enzyme is a Sleeping Beauty transposase enzyme (see, for example, U.S. Pat. No. 9,228,180, the contents of which are incorporated herein in their entirety). In some embodiments, the Sleeping Beauty transposase is a hyperactive Sleeping Beauty (SB100X) transposase.

The Helraiser transposon is transposed by the Helitron transposase. Unlike other transposases, the Helitron transposase does not contain an RNase-H like catalytic domain, but instead comprises a RepHel motif made up of a replication initiator domain (Rep) and a DNA helicase domain. The Rep domain is a nuclease domain of the HUH superfamily of nucleases. In some embodiments, the transposon is a Helraiser transposon. In some embodiments of the Helraiser transposon sequence, the transposase is flanked by left and right terminal sequences termed LTS and RTS. In some embodiments, these sequences terminate with a conserved 5′-TC/CTAG-3′ motif. In some embodiments, a 19 bp palindromic sequence with the potential to form the hairpin termination structure is located 11 nucleotides upstream of the RTS and comprises the sequence GTGCACGAATTTCGTGCACCGGGCCACTAG. In some embodiments, and, in particular embodiments wherein the transposon is a Helraiser transposon, the transposase enzyme is a Helitron transposase enzyme.

Tol2 transposons may be isolated or derived from the genome of the medaka fish, and may be similar to transposons of the hAT family. Exemplary Tol2 transposons of the disclosure are encoded by a sequence comprising about 4.7 kilobases and contain a gene encoding the Tol2 transposase, which contains four exons. In some embodiments, the transposon is a Tol2 transposon. In certain embodiments of the methods of the disclosure, and, in particular those embodiments wherein the transposon is a Tol2 transposon, the transposase enzyme is a Tol2 transposase enzyme.

In some embodiments, a vector comprises the recombinant and non-naturally occurring DNA sequence encoding the transposon. In some embodiments, the vector comprises any form of DNA and wherein the vector comprises at least 100 nucleotides (nts), 500 nts, 1000 nts, 1500 nts, 2000 nts, 2500 nts, 3000 nts, 3500 nts, 4000 nts, 4500 nts, 5000 nts, 6500 nts, 7000 nts, 7500 nts, 8000 nts, 8500 nts, 9000 nts, 9500 nts, 10,000 nts or any number of nucleotides in between. In some embodiments, the vector comprises single-stranded or double-stranded DNA. In some embodiments, the vector comprises circular DNA. In some embodiments, the vector is a plasmid vector, a nanoplasmid vector, a minicircle. In some embodiments, the vector comprises linear or linearized DNA. In some embodiments, the vector is a double-stranded Doggybone™ DNA sequence.

In some embodiments, the recombinant and non-naturally occurring DNA sequence encoding a transposon further comprises a sequence encoding one or more immune checkpoint genes.

In some embodiments, the nucleic acid sequence encoding the transposase enzyme is a DNA sequence, and an amount of the DNA sequence encoding the transposase enzyme and an amount of the DNA sequence encoding the transposon is equal to or less than 10.0 μg per 100 μL, less than 7.5 μg per 100 μL, less than 6.0 μg per 100 μL, less than 5.0 μg per 100 μL, less than 2.5 μg per 100 μL, or less than 1.67 μg per 100 μL, less than 0.55 μg per 100 μL, less than 0.19 μg per 100 μL, less than 0.10 μg per 100 μL of an electroporation or nucleofection reaction. In certain embodiments, a concentration of the amount of the DNA sequence encoding the transposase enzyme and an amount of the DNA sequence encoding the transposon in the electroporation or nucleofection reaction is equal to or less than 100 μg/mL, equal to or less than 75 μg/mL, equal to or less than 60 μg/mL, equal to or less than 50 μg/mL, equal to or less than 25 μg/mL, equal to or less than 16.7 μg/mL, equal to or less than 5.5 μg/mL, equal to or less than 1.9 μg/mL, equal to or less than 1.0 μg/mL.

In some embodiments, the nucleic acid sequence encoding the transposase enzyme is an RNA sequence, and an amount of the RNA sequence encoding the transposase enzyme and an amount of the RNA sequence encoding the transposon is equal to or less than 10.0 μg per 100 μL, less than 7.5 μg per 100 μL, less than 6.0 μg per 100 μL, less than 5.0 μg per 100 μL, less than 2.5 μg per 100 μL, or less than 1.67 μg per 100 μL, less than 0.55 μg per 100 μL, less than 0.19 μg per 100 μL, less than 0.10 μg per 100 μL of an electroporation or nucleofection reaction. In certain embodiments, a concentration of the amount of the RNA sequence encoding the transposase enzyme and an amount of the RNA sequence encoding the transposon in the electroporation or nucleofection reaction is equal to or less than 100 μg/mL, equal to or less than 75 μg/mL, equal to or less than 60 μg/mL, equal to or less than 50 μg/mL, equal to or less than 25 μg/mL, equal to or less than 16.7 μg/mL, equal to or less than 5.5 μg/mL, equal to or less than 1.9 μg/mL, equal to or less than 1.0 μg/mL.

In some embodiments, the TILs are further modified by a second gene editing tool, including, but not limited to those described herein. In some embodiments, the second gene editing tool may include an excision-only piggyBac transposase to re-excise the inserted sequences or any portion thereof. For example, the excision-only piggyBac transposase may be used to “re-excise” the transposon.

According to some embodiments, a piggyBac system comprises a transposon/transposase system, wherein the transposase recognizes the ITRs located on both sides of the transposon cassette comprising a cargo encoding one or more immune checkpoint genes, and excises the transposable element to integrate it into TT/AA chromosomal sites, resulting in genomic insertion of the transposon cassette and expression of the one or more immune checkpoint genes. According to some embodiments, the cargo encodes two or more immune checkpoint molecules.

Non-limiting examples of genes that may be enhanced by permanently gene-editing TILs via a piggyBac method include CCR2, CCR4, CCR5, CXCR2, CXCR3, CX3CR1, IL-2, IL-4, IL-7, IL-10, IL-15, IL-18, IL-21, the NOTCH 1/2 intracellular domain (ICD), and/or the NOTCH ligand mDLL1. Additional genes that may be enhanced by gene-editing TILs via a piggyBac method include immune checkpoint genes such, such as, PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

Examples of systems, methods, and compositions for altering the expression of a target gene sequence by a piggyBac method, and which may be used in accordance with embodiments of the present invention, are described in WO2019/046815, WO2015006700, WO2010085699, WO2010099301, WO2010099296, WO2006122442, WO2001081565, and WO1998040510, the contents of which are incorporated herein by reference in their entirety.

Resources for carrying out piggyBac methods, such as plasmids for expressing transposons/transposases, are commercially available from companies such as Demeetra and Hera Biolabs.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system comprising a piggyBac system, which at least one gene editor system effects expression of at least one immunomodulatory composition at the cell surface of and modulates expression of at least one checkpoint protein in the plurality of cells of the second population of TILs.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system comprising a piggyBac system, which at least one gene editor system effects expression of at least one immunomodulatory composition at the cell surface of in the plurality of cells of the second population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, a method of genetically modifying a population of TILs includes the use of a non-viral technique such as a piggyBac method (e.g., piggyBac transposons and transposases or piggyBac-like transposons and transposases). In some embodiments, the method comprises delivering to the TILs: (a) a nucleic acid or amino acid sequence comprising a sequence encoding a transposase enzyme; and (b) a recombinant and non-naturally occurring DNA sequence comprising a DNA sequence encoding a transposon. In certain embodiments of the methods of the disclosure, the sequence encoding a transposase enzyme is an mRNA sequence. The mRNA sequence encoding a transposase enzyme may be produced in vitro. In certain embodiments of the methods of the disclosure, the sequence encoding a transposase enzyme is a DNA sequence. The DNA sequence encoding a transposase enzyme may be produced in vitro. The DNA sequence may be a cDNA sequence. In certain embodiments of the methods of the disclosure, the sequence encoding a transposase enzyme is an amino acid sequence. The amino acid sequence encoding a transposase enzyme may be produced in vitro. A protein Super piggybac transposase (SPB) may be delivered following pre-incubation with transposon DNA. In certain embodiments, the transposon is a piggyBac transposon or a piggyBac-like transposon. In certain embodiments, and, in particular, those embodiments wherein the transposon is a piggyBac transposon, the transposase is a piggyBac transposase. In certain embodiments, and, in particular, those embodiments wherein the transposon is a piggyBac-like transposon, the transposase is a piggyBac-like transposase. In certain embodiments, the piggyBac transposase comprises an amino acid sequence comprising SEQ ID NO:387 (SEQ ID NO: 14487 of WO2019046815). In certain embodiments, and, in particular, those embodiments wherein the transposon is a piggyBac transposon, the transposase is a piggyBac™ or a Super piggyBac™ (SPB) transposase. In certain embodiments, and, in particular, those embodiments wherein the transposase is a Super piggyBac™ (SPB) transposase, the sequence encoding the transposase is an mRNA sequence. In certain embodiments of the methods of the disclosure, the transposase enzyme is a piggyBac™ (PB) transposase enzyme. The piggyBac (PB) transposase enzyme may comprise or consist of an amino acid sequence at least 75%, 80%, 85%, 90%, 95%, 99% or any percentage in between identical to:
MGSSLDDEHILSALLQSDDELVGEDSDSEISDHVSEDDVQSDTEEAFIDEVHEVQPTSSGSEILDEQN VIEQPGSSLASNRILTLPQRTIRGKNKHCWSTSKSTRRSRVSALNIVRSQRGPTRMCRNIYDPLLCFKL FFTDEIISEIVKWTNAEISLKRRESMTGATFRDTNEDEIYAFFGILVMTAVRKDNHMSTDDLFDRSLSM VYVSVMSRDRFDFLIRCLRMDDKSIRPTLRENDVFTPVRKIWDLFIHQCIQNYTPGAHLTIDEQLLGFR GRCPFRMYIPNKPSKYGIKILMMCDSGTKYMINGMPYLGRGTQTNGVPLGEYYVKELSKPVHGSCRN ITCDNWFTSIPLAKNLLQEPYKLTIVGTVRSNKREIPEVLKNSRSRPVGTSMFCFDGPLTLVSYKPKPA KMVYLLSSCDEDASINESTGKPQMVMYYNQTKGGVDTLDQMCSVMTCSRKTNRWPMALLYGMINIA CINSFIIYSHNVSSKGEKVQSRKKFMRNLYMSLTSSFMRKRLEAPTLKRYLRDNISNILPNEVPGTSDD STEEPVMKKRTYCTYCPSKIRRKANASCKKCKKVICREHNIDMCQSCF (SEQ ID NO:387; SEQ ID NO: 14487 of WO2019046815).

In certain embodiments of the methods of the disclosure, the transposon is a Sleeping Beauty transposon. In certain embodiments of the methods of the disclosure, the transposase enzyme is a Sleeping Beauty transposase enzyme (see, for example, U.S. Pat. No. 9,228,180, the contents of which are incorporated herein in their entirety). In certain embodiments, the Sleeping Beauty transposase is a hyperactive Sleeping Beauty (SB100X) transposase. In certain embodiments, the Sleeping Beauty transposase enzyme comprises an amino acid sequence at least 75%, 80%, 85%, 90%, 95%, 99% or any percentage in between identical to:
MGKSKEISQDLRKKIVDLHKSGSSLGAISKRLKVPRSSVQTIVRKYKHHGTTQPSYRSGRRRVLSPRD ERTLVRKVQINPRTTAKDLVKMLEETGTKVSISTVKRVLYRHNLKGRSARKKPLLQNRHKKARLRFAT AHGDKDRTFWRNVLWSDETKIELFGHNDHRYVWRKKGEACKPKNTIPTVKHGGGSIMLWGCFAAG GTGALHKIDGIMRKENYVDILKQHLKTSVRKLKLGRKWVFQMDNDPKHTSKVVAKWLKDNKVKVLEW PSQSPDLNPIENLWAELKKRVRARRPTNLTQLHQLCQEEWAKIHPTYCGKLVEGYPKRLTQVKQFKG NATKY (SEQ ID NO: 388; SEQ ID NO: 14485 of WO2019046815).

In certain embodiments, including those wherein the Sleeping Beauty transposase is a hyperactive Sleeping Beauty (SB100X) transposase, the Sleeping Beauty transposase enzyme comprises an amino acid sequence at least at least 75%, 80%, 85%, 90%, 95%, 99% or any percentage in between identical to:
MGKSKEISQDLRKRIVDLHKSGSSLGAISKRLAVPRSSVQTIVRKYKHHGTTQPSYRSGRRRVLSPRD ERTLVRKVQINPRTTAKDLVKMLEETGTKVSISTVKRVLYRHNLKGHSARKKPLLQNRHKKARLRFAT AHGDKDRTFWRNVLWSDETKIELFGHNDHRYVWRKKGEACKPKNTIPTVKHGGGSIMLWGCFAAG GTGALHKIDGIMDAVQYVDILKQHLKTSVRKLKLGRKWVFQHDNDPKHTSKVVAKWLKDNKVKVLEW PSQSPDLNPIENLWAELKKRVRARRPTNLTQLHQLCQEEWAKIHPNYCGKLVEGYPKRLTQVKQFKG NATKY (SEQ ID NO: 389; SEQ ID NO: 14486 of WO2019046815).

### 1. CRISPR Methods

A method for expanding TILs into a therapeutic population may be carried out in accordance with any embodiment of the methods described herein (e.g., process 2A) or as described in PCT/US2017/058610, PCT/US2018/012605, or PCT/US2018/012633, wherein the method further comprises gene-editing at least a portion of the TILs by a CRISPR method (e.g., CRISPR/Cas9 or CRISPR/Cpf1). According to particular embodiments, the use of a CRISPR method during the TIL expansion process causes expression of at least one immunomodulatory composition at the cell surface of, and optionally causes one or more immune checkpoint genes to be silenced or reduced in, at least a portion of the therapeutic population of TILs. Alternatively, the use of a CRISPR method during the TIL expansion process causes expression of at least one immunomodulatory composition at the cell surface of, and optionally causes one or more immune checkpoint genes to be enhanced in, at least a portion of the therapeutic population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

CRISPR stands for “Clustered Regularly Interspaced Short Palindromic Repeats.” A method of using a CRISPR system for gene editing is also referred to herein as a CRISPR method. CRISPR systems can be divided into two main classes, Class 1 and Class 2, which are further classified into different types and sub-types. The classification of the CRISPR systems is based on the effector Cas proteins that are capable of cleaving specific nucleic acids. In Class 1 CRISPR systems the effector module consists of a multi-protein complex, whereas Class 2 systems only use one effector protein. Class 1 CRISPR includes Types I, III, and IV and Class 2 CRISPR includes Types II, V, and VI. While any of these types of CRISPR systems may be used in accordance with the present invention, there are three types of CRISPR systems which incorporate RNAs and Cas proteins that are preferred for use in accordance with the present invention: Types I (exemplified by Cas3), II (exemplified by Cas9), and III (exemplified by Cas10). The Type II CRISPR is one of the most well-characterized systems.

CRISPR technology was adapted from the natural defense mechanisms of bacteria and archaea (the domain of single-celled microorganisms). These organisms use CRISPR-derived RNA and various Cas proteins, including Cas9, to foil attacks by viruses and other foreign bodies by chopping up and destroying the DNA of a foreign invader. A CRISPR is a specialized region of DNA with two distinct characteristics: the presence of nucleotide repeats and spacers. Repeated sequences of nucleotides are distributed throughout a CRISPR region with short segments of foreign DNA (spacers) interspersed among the repeated sequences. In the type II CRISPR/Cas system, spacers are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Target recognition by the Cas9 protein requires a “seed” sequence within the crRNA and a conserved dinucleotide-containing protospacer adjacent motif (PAM) sequence upstream of the crRNA-binding region. The CRISPR/Cas system can thereby be retargeted to cleave virtually any DNA sequence by redesigning the crRNA. Thus, according to certain embodiments, Cas9 serves as an RNA-guided DNA endonuclease that cleaves DNA upon crRNA-tracrRNA recognition.

The crRNA and tracrRNA in the native system can be simplified into a single guide RNA (sgRNA) of approximately 100 nucleotides for use in genetic engineering. The sgRNA is a synthetic RNA that includes a scaffold sequence necessary for Cas-binding and a user-defined approximately 17- to 20-nucleotide spacer that defines the genomic target to be modified. Thus, a user can change the genomic target of the Cas protein by changing the target sequence present in the sgRNA. The CRISPR/Cas system is directly portable to human cells by co-delivery of plasmids expressing the Cas9 endo-nuclease and the RNA components (e.g., sgRNA). Different variants of Cas proteins may be used to reduce targeting limitations (e.g., orthologs of Cas9, such as Cpf1).

According to some embodiments, an engineered, programmable, non-naturally occurring Type II CRISPR-Cas system comprises a Cas9 protein and at least one guide RNA that targets and hybridizes to a target sequence of a DNA molecule in a TIL, wherein the DNA molecule encodes and the TIL expresses at least one immune checkpoint molecule, and the Cas9 protein cleaves the DNA molecules, whereby expression of the at least one immune checkpoint molecule is altered; and, wherein the Cas9 protein and the guide RNA do not naturally occur together. According to some embodiments, the expression of two or more immune checkpoint molecules is altered. According to some embodiments, the guide RNA(s) comprise a guide sequence fused to a tracr sequence. For example, the guide RNA may comprise crRNA-tracrRNA or sgRNA. According to aspects of the present invention, the terms “guide RNA”, “single guide RNA” and “synthetic guide RNA” may be used interchangeably and refer to the polynucleotide sequence comprising the guide sequence, which is the approximately 17-20 bp sequence within the guide RNA that specifies the target site.

Variants of Cas9 having improved on-target specificity compared to Cas9 may also be used in accordance with embodiments of the present invention. Such variants may be referred to as high-fidelity Cas-9s. According to some embodiments, a dual nickase approach may be utilized, wherein two nickases targeting opposite DNA strands generate a DSB within the target DNA (often referred to as a double nick or dual nickase CRISPR system). For example, this approach may involve the mutation of one of the two Cas9 nuclease domains, turning Cas9 from a nuclease into a nickase. Non-limiting examples of high-fidelity Cas9s include eSpCas9, SpCas9-HF1 and HypaCas9. Such variants may reduce or eliminate unwanted changes at non-target DNA sites. See, e.g., Slaymaker I M, et al. Science. 2015 Dec. 1, Kleinstiver B P, et al. Nature. 2016 Jan. 6, and Ran et al., Nat Protoc. 2013 November; 8(11):2281-2308, the disclosures of which are incorporated by reference herein.

Additionally, according to particular embodiments, Cas9 scaffolds may be used that improve gene delivery of Cas9 into cells and improve on-target specificity, such as those disclosed in U.S. Patent Application Publication No. 2016/0102324, which is incorporated by reference herein. For example, Cas9 scaffolds may include a RuvC motif as defined by (D-[I/L]-G-X-X-S-X-G-W-A) and/or a HNH motif defined by (Y-X-X-D-H-X-X-P-X-S-X-X-X-D-X-S), where X represents any one of the 20 naturally occurring amino acids and [I/L] represents isoleucine or leucine. The HNH domain is responsible for nicking one strand of the target dsDNA and the RuvC domain is involved in cleavage of the other strand of the dsDNA. Thus, each of these domains nick a strand of the target DNA within the protospacer in the immediate vicinity of PAM, resulting in blunt cleavage of the DNA. These motifs may be combined with each other to create more compact and/or more specific Cas9 scaffolds. Further, the motifs may be used to create a split Cas9 protein (i.e., a reduced or truncated form of a Cas9 protein or Cas9 variant that comprises either a RuvC domain or a HNH domain) that is divided into two separate RuvC and HNH domains, which can process the target DNA together or separately.

According to particular embodiments, a CRISPR method comprises silencing or reducing the expression of one or more immune checkpoint genes in TILs by introducing a Cas9 nuclease and a guide RNA (e.g., crRNA-tracrRNA or sgRNA) containing a sequence of approximately 17-20 nucleotides specific to a target DNA sequence of the immune checkpoint gene(s). The guide RNA may be delivered as RNA or by transforming a plasmid with the guide RNA-coding sequence under a promoter. The CRISPR/Cas enzymes introduce a double-strand break (DSB) at a specific location based on a sgRNA-defined target sequence. DSBs may be repaired in the cells by non-homologous end joining (NHEJ), a mechanism which frequently causes insertions or deletions (indels) in the DNA. Indels often lead to frameshifts, creating loss of function alleles; for example, by causing premature stop codons within the open reading frame (ORF) of the targeted gene. According to certain embodiments, the result is a loss-of-function mutation within the targeted immune checkpoint gene.

Alternatively, DSBs induced by CRISPR/Cas enzymes may be repaired by homology-directed repair (HDR) instead of NHEJ. While NHEJ-mediated DSB repair often disrupts the open reading frame of the gene, homology directed repair (HDR) can be used to generate specific nucleotide changes ranging from a single nucleotide change to large insertions. According to some embodiments, HDR is used for gene editing immune checkpoint genes by delivering a DNA repair template containing the desired sequence into the TILs with the sgRNA(s) and Cas9 or Cas9 nickase. The repair template preferably contains the desired edit as well as additional homologous sequence immediately upstream and downstream of the target gene (often referred to as left and right homology arms).

According to particular embodiments, an enzymatically inactive version of Cas9 (deadCas9 or dCas9) may be targeted to transcription start sites in order to repress transcription by blocking initiation. Thus, targeted immune checkpoint genes may be repressed without the use of a DSB. A dCas9 molecule retains the ability to bind to target DNA based on the sgRNA targeting sequence. According to some embodiments of the present invention, a CRISPR method comprises silencing or reducing the expression of one or more immune checkpoint genes by inhibiting or preventing transcription of the targeted gene(s). For example, a CRISPR method may comprise fusing a transcriptional repressor domain, such as a Kruppel-associated box (KRAB) domain, to an enzymatically inactive version of Cas9, thereby forming, e.g., a dCas9-KRAB, that targets the immune checkpoint gene's transcription start site, leading to the inhibition or prevention of transcription of the gene. Preferably, the repressor domain is targeted to a window downstream from the transcription start site, e.g., about 500 bp downstream. This approach, which may be referred to as CRISPR interference (CRISPRi), leads to robust gene knockdown via transcriptional reduction of the target RNA.

According to particular embodiments, an enzymatically inactive version of Cas9 (deadCas9 or dCas9) may be targeted to transcription start sites in order to activate transcription. This approach may be referred to as CRISPR activation (CRISPRa). According to some embodiments, a CRISPR method comprises increasing the expression of one or more immune checkpoint genes by activating transcription of the targeted gene(s). According to such embodiments, targeted immune checkpoint genes may be activated without the use of a DSB. A CRISPR method may comprise targeting transcriptional activation domains to the transcription start site; for example, by fusing a transcriptional activator, such as VP64, to dCas9, thereby forming, e.g., a dCas9-VP64, that targets the immune checkpoint gene's transcription start site, leading to activation of transcription of the gene. Preferably, the activator domain is targeted to a window upstream from the transcription start site, e.g., about 50-400 bp downstream

Additional embodiments of the present invention may utilize activation strategies that have been developed for potent activation of target genes in mammalian cells. Non-limiting examples include co-expression of epitope-tagged dCas9 and antibody-activator effector proteins (e.g., the SunTag system), dCas9 fused to a plurality of different activation domains in series (e.g., dCas9-VPR) or co-expression of dCas9-VP64 with a modified scaffold gRNA and additional RNA-binding helper activators (e.g., SAM activators).

According to other embodiments, a CRISPR-mediated genome editing method referred to as CRISPR assisted rational protein engineering (CARPE) may be used in accordance with embodiments of the present invention, as disclosed in U.S. Pat. No. 9,982,278, which is incorporated by reference herein. CARPE involves the generation of “donor” and “destination” libraries that incorporate directed mutations from single-stranded DNA (ssDNA) or double-stranded DNA (dsDNA) editing cassettes directly into the genome. Construction of the donor library involves cotransforming rationally designed editing oligonucleotides into cells with a guide RNA (gRNA) that hybridizes to a target DNA sequence. The editing oligonucleotides are designed to couple deletion or mutation of a PAM with the mutation of one or more desired codons in the adjacent gene. This enables the entire donor library to be generated in a single transformation. The donor library is retrieved by amplification of the recombinant chromosomes, such as by a PCR reaction, using a synthetic feature from the editing oligonucleotide, namely, a second PAM deletion or mutation that is simultaneously incorporated at the 3′ terminus of the gene. This covalently couples the codon target mutations directed to a PAM deletion. The donor libraries are then co-transformed into cells with a destination gRNA vector to create a population of cells that express a rationally designed protein library.

According to other embodiments, methods for trackable, precision genome editing using a CRISPR-mediated system referred to as Genome Engineering by Trackable CRISPR Enriched Recombineering (GEn-TraCER) may be used in accordance with embodiments of the present invention, as disclosed in U.S. Pat. No. 9,982,278, which is incorporated by reference herein. The GEn-TraCER methods and vectors combine an editing cassette with a gene encoding gRNA on a single vector. The cassette contains a desired mutation and a PAM mutation. The vector, which may also encode Cas9, is the introduced into a cell or population of cells. This activates expression of the CRISPR system in the cell or population of cells, causing the gRNA to recruit Cas9 to the target region, where a dsDNA break occurs, allowing integration of the PAM mutation.

Non-limiting examples of genes that may be silenced or inhibited by permanently gene-editing TILs via a CRISPR method include PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

Non-limiting examples of genes that may be enhanced by permanently gene-editing TILs via a CRISPR method include CCR2, CCR4, CCR5, CXCR2, CXCR3, CX3CR1, IL-2, IL-4, IL-7, IL-10, IL-15, IL-18, IL-21, the NOTCH 1/2 intracellular domain (ICD), and/or the NOTCH ligand mDLL1.

Examples of systems, methods, and compositions for altering the expression of a target gene sequence by a CRISPR method, and which may be used in accordance with embodiments of the present invention, are described in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and 8,895,308, which are incorporated by reference herein. Resources for carrying out CRISPR methods, such as plasmids for expressing CRISPR/Cas9 and CRISPR/Cpf1, are commercially available from companies such as GenScript.

In some embodiments, genetic modifications of populations of TILs, as described herein, may be performed using the CRISPR/Cpf1 system as described in U.S. Pat. No. 9,790,490, the disclosure of which is incorporated by reference herein. The CRISPR/Cpf1 system is functionally distinct from the CRISPR-Cas9 system in that Cpf1-associated CRISPR arrays are processed into mature crRNAs without the need for an additional tracrRNA. The crRNAs used in the CRISPR/Cpf1 system have a spacer or guide sequence and a direct repeat sequence. The Cpf1p-crRNA complex that is formed using this method is sufficient by itself to cleave the target DNA.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system selected from the group consisting of a Clustered Regularly Interspersed Short Palindromic Repeat (CRISPR)/Cas9 system and a CRISPR/Cpf1 system, which at least one gene editor system effects expression of at least one immunomodulatory composition at the cell surface of and modulates expression of at least one checkpoint protein in the plurality of cells of the second population of TILs.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system selected from the group consisting of a Clustered Regularly Interspersed Short Palindromic Repeat (CRISPR)/Cas9 system and a CRISPR/Cpf1 system, which at least one gene editor system effects expression of at least one immunomodulatory composition at the cell surface of and inhibits expression of PD-1 and LAG-3 in the plurality of cells of the second population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist. 2. TALE Methods

A method for expanding TILs into a therapeutic population may be carried out in accordance with any embodiment of the methods described herein (e.g., process 2A) or as described in WO2018081473, WO2018129332, or WO2018182817, wherein the method further comprises gene-editing at least a portion of the TILs by a TALE method. According to particular embodiments, the use of a TALE method during the TIL expansion process causes expression of at least one immunomodulatory composition at the cell surface, and optionally causes expression of one or more immune checkpoint genes to be silenced or reduced, in at least a portion of the therapeutic population of TILs. Alternatively, the use of a TALE method during the TIL expansion process causes expression of at least one immunomodulatory composition at the cell surface, and optionally causes expression of one or more immune checkpoint genes to be enhanced, in at least a portion of the therapeutic population of TILs.

TALE stands for “Transcription Activator-Like Effector” proteins, which include TALENs (“Transcription Activator-Like Effector Nucleases”). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Strategies that enable the rapid assembly of custom TALE arrays include Golden Gate molecular cloning, high-throughput solid-phase assembly, and ligation-independent cloning techniques. Custom-designed TALE arrays are also commercially available through Cellectis Bioresearch (Paris, France), Transposagen Biopharmaceuticals (Lexington, KY, USA), and Life Technologies (Grand Island, NY, USA). Additionally web-based tools, such as TAL Effector-Nucleotide Target 2.0, are available that enable the design of custom TAL effector repeat arrays for desired targets and also provides predicted TAL effector binding sites. See Doyle, et al., Nucleic Acids Research, 2012, Vol. 40, W117-W122. Examples of TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of which are incorporated by reference herein.

According to some embodiments of the present invention, a TALE method comprises silencing or reducing the expression of one or more immune checkpoint genes by inhibiting or preventing transcription of the targeted gene(s). For example, a TALE method may include utilizing KRAB-TALEs, wherein the method comprises fusing a transcriptional Kruppel-associated box (KRAB) domain to a DNA binding domain that targets the gene's transcription start site, leading to the inhibition or prevention of transcription of the gene.

According to other embodiments, a TALE method comprises silencing or reducing the expression of one or more immune checkpoint genes by introducing mutations in the targeted gene(s). For example, a TALE method may include fusing a nuclease effector domain, such as Fokl, to the TALE DNA binding domain, resulting in a TALEN. Fokl is active as a dimer; hence, the method comprises constructing pairs of TALENs to position the FOKL nuclease domains to adjacent genomic target sites, where they introduce DNA double strand breaks. A double strand break may be completed following correct positioning and dimerization of Fokl. Once the double strand break is introduced, DNA repair can be achieved via two different mechanisms: the high-fidelity homologous recombination pair (HRR) (also known as homology-directed repair or HDR) or the error-prone non-homologous end joining (NHEJ). Repair of double strand breaks via NHEJ preferably results in DNA target site deletions, insertions or substitutions, i.e., NHEJ typically leads to the introduction of small insertions and deletions at the site of the break, often inducing frameshifts that knockout gene function. According to particular embodiments, the TALEN pairs are targeted to the most 5′ exons of the genes, promoting early frame shift mutations or premature stop codons. The genetic mutation(s) introduced by TALEN are preferably permanent. Thus, according to some embodiments, the method comprises silencing or reducing expression of an immune checkpoint gene by utilizing dimerized TALENs to induce a site-specific double strand break that is repaired via error-prone NHEJ, leading to one or more mutations in the targeted immune checkpoint gene.

According to additional embodiments, TALENs are utilized to introduce genetic alterations via HRR, such as non-random point mutations, targeted deletion, or addition of DNA fragments. The introduction of DNA double strand breaks enables gene editing via homologous recombination in the presence of suitable donor DNA. According to some embodiments, the method comprises co-delivering dimerized TALENs and a donor plasmid bearing locus-specific homology arms to induce a site-specific double strand break and integrate one or more transgenes into the DNA.

According to other embodiments, a TALEN that is a hybrid protein derived from FokI and AvrXa7, as disclosed in U.S. Patent Publication No. 2011/0201118, may be used in accordance with embodiments of the present invention. This TALEN retains recognition specificity for target nucleotides of AvrXa7 and the double-stranded DNA cleaving activity of FokI. The same methods can be used to prepare other TALEN having different recognition specificity. For example, compact TALENs may be generated by engineering a core TALE scaffold having different sets of RVDs to change the DNA binding specificity and target a specific single dsDNA target sequence. See U.S. Patent Publication No. 2013/0117869. A selection of catalytic domains can be attached to the scaffold to effect DNA processing, which may be engineered to ensure that the catalytic domain is capable of processing DNA near the single dsDNA target sequence when fused to the core TALE scaffold. A peptide linker may also be engineered to fuse the catalytic domain to the scaffold to create a compact TALEN made of a single polypeptide chain that does not require dimerization to target a specific single dsDNA sequence. A core TALE scaffold may also be modified by fusing a catalytic domain, which may be a TAL monomer, to its N-terminus, allowing for the possibility that this catalytic domain might interact with another catalytic domain fused to another TAL monomer, thereby creating a catalytic entity likely to process DNA in the proximity of the target sequences. See U.S. Patent Publication No. 2015/0203871. This architecture allows only one DNA strand to be targeted, which is not an option for classical TALEN architectures.

According to some embodiments of the present invention, conventional RVDs may be used create TALENs that are capable of significantly reducing gene expression. In some embodiments, four RVDs, NI, HD, NN, and NG, are used to target adenine, cytosine, guanine, and thymine, respectively. These conventional RVDs can be used to, for instance, create TALENs targeting the PD-1 gene. Examples of TALENs using conventional RVDs include the T3v1 and T1 TALENs disclosed in Gautron et al., Molecular Therapy: Nucleic Acids December 2017, Vol. 9:312-321 (Gautron), which is incorporated by reference herein. The T3v1 and T1 TALENs target the second exon of the PDCD1 locus where the PD-L1 binding site is located and are able to considerably reduce PD-1 production. In some embodiments, the T1 TALEN does so by using target SEQ ID NO:256 and the T3v1 TALEN does so by using target SEQ ID NO:257.

According to other embodiments, TALENs are modified using non-conventional RVDs to improve their activity and specificity for a target gene, such as disclosed in Gautron. Naturally occurring RVDs only cover a small fraction of the potential diversity repertoire for the hypervariable amino acid locations. Non-conventional RVDs provide an alternative to natural RVDs and have novel intrinsic targeting specificity features that can be used to exclude the targeting of off-site targets (sequences within the genome that contain a few mismatches relative to the targeted sequence) by TALEN. Non-conventional RVDs may be identified by generating and screening collections of TALEN containing alternative combinations of amino acids at the two hypervariable amino acid locations at defined positions of an array as disclosed in Juillerat, et al., Scientific Reports 5, Article Number 8150 (2015), which is incorporated by reference herein. Next, non-conventional RVDs may be selected that discriminate between the nucleotides present at the position of mismatches, which can prevent TALEN activity at off-site sequences while still allowing appropriate processing of the target location. The selected non-conventional RVDs may then be used to replace the conventional RVDs in a TALEN. Examples of TALENs where conventional RVDs have been replaced by non-conventional RVDs include the T3v2 and T3v3 PD-1 TALENs produced by Gautron. These TALENs had increased specificity when compared to TALENs using conventional RVDs.

According to additional embodiments, TALEN may be utilized to introduce genetic alterations to silence or reduce the expression of two genes. For instance, two separate TALEN may be generated to target two different genes and then used together. The molecular events generated by the two TALEN at their respective loci and potential off-target sites may be characterized by high-throughput DNA sequencing. This enables the analysis of off-target sites and identification of the sites that might result from the use of both TALEN. Based on this information, appropriate conventional and non-conventional RVDs may be selected to engineer TALEN that have increased specificity and activity even when used together. For example, Gautron discloses the combined use of T3v4 PD-1 and TRAC TALEN to produce double knockout CAR T cells, which maintained a potent in vitro anti-tumor function.

In some embodiments, the method of Gautron or other methods described herein may be employed to genetically-edit TILs, which may then be expanded by any of the procedures described herein. In some embodiments, a method for expanding tumor infiltrating lymphocytes (TILs) into a therapeutic population of TILs comprises the steps of:
(a) activating a first population of TILs obtained from a tumor resected from a patient using CD3 and CD28 activating beads or antibodies for 1 to 5 days;
(b) gene-editing at least a portion of the first population of TILs using electroporation of transcription activator-like effector nuclease-encoding nucleic acids to obtain a second population of TILs, wherein the gene-editing effects expression of at least one immunomodulatory composition at the cell surface, and inhibits expression of at least one immune checkpoint protein, in the portion of the cells of the second population of TILs;
(c) optionally incubating the second population of TILs;
(d) performing a first expansion by culturing the second population of TILs in a cell culture medium comprising IL-2, and optionally OKT-3, to produce a third population of TILs, wherein the first expansion is performed in a closed container providing a first gas-permeable surface area, wherein the first expansion is performed for about 3 to 14 days to obtain the third population of TILs;
(e) performing a second expansion by supplementing the cell culture medium of the third population of TILs with additional IL-2, OKT-3, and antigen presenting cells (APCs), to produce a fourth population of TILs, wherein the second expansion is performed for about 7 to 14 days to obtain the fourth population of TILs, wherein the fourth population of TILs is a therapeutic population of TILs;
(f) harvesting the therapeutic population of TILs obtained from step (e);
(g) transferring the harvested TIL population from step (e) to an infusion bag, wherein the transfer from step (e) to (f) occurs without opening the system; and
(h) wherein one or more of steps (a) to (g) are performed in a closed, sterile system.

In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, a method for expanding tumor infiltrating lymphocytes (TILs) into a therapeutic population of TILs comprises the steps of:
(a) activating a first population of TILs obtained from a tumor resected from a patient using CD3 and CD28 activating beads or antibodies for 1 to 5 days;
(b) gene-editing at least a portion of the first population of TILs using electroporation of transcription activator-like effector nuclease-encoding nucleic acids in cytoporation medium to obtain a second population of TILs, wherein the gene-editing effects expression of at least one immunomodulatory composition at the cell surface, and inhibits expression of at least one immune checkpoint protein, in the portion of the cells of the second population of TILs;
(c) optionally incubating the second population of TILs;
(d) performing a first expansion by culturing the second population of TILs in a cell culture medium comprising IL-2, and optionally OKT-3, to produce a third population of TILs, wherein the first expansion is performed in a closed container providing a first gas-permeable surface area, wherein the first expansion is performed for about 6 to 9 days to obtain the third population of TILs;
(e) performing a second expansion by supplementing the cell culture medium of the third population of TILs with additional IL-2, OKT-3, and antigen presenting cells (APCs), to produce a fourth population of TILs, wherein the second expansion is performed for about 9 to 11 days to obtain the fourth population of TILs, wherein the fourth population of TILs is a therapeutic population of TILs;
(f) harvesting the therapeutic population of TILs obtained from step (e);
(g) transferring the harvested TIL population from step (e) to an infusion bag, wherein the transfer from step (e) to (f) occurs without opening the system; and
(h) wherein one or more of steps (a) to (g) are performed in a closed, sterile system.
   In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, a method for expanding tumor infiltrating lymphocytes (TILs) into a therapeutic population of TILs comprises the steps of:
(a) activating a first population of TILs obtained from a tumor resected from a patient using CD3 and CD28 activating beads or antibodies for 1 to 5 days;
(b) gene-editing at least a portion of the first population of TILs using electroporation of transcription activator-like effector nuclease-encoding nucleic acids in cytoporation medium to obtain a second population of TILs, wherein the gene-editing effects expression of at least one immunomodulatory composition at the cell surface, and inhibits expression of at least one immune checkpoint protein, in the portion of the cells of the second population of TILs;
(c) optionally incubating the second population of TILs, wherein the incubation is performed at about 30-40° C. with about 5% CO₂;
(d) performing a first expansion by culturing the second population of TILs in a cell culture medium comprising IL-2, and optionally OKT-3, to produce a third population of TILs, wherein the first expansion is performed in a closed container providing a first gas-permeable surface area, wherein the first expansion is performed for about 6 to 9 days to obtain the third population of TILs;
(e) performing a second expansion by supplementing the cell culture medium of the third population of TILs with additional IL-2, OKT-3, and antigen presenting cells (APCs), to produce a fourth population of TILs, wherein the second expansion is performed for about 9 to 11 days to obtain the fourth population of TILs, wherein the fourth population of TILs is a therapeutic population of TILs;
(f) harvesting the therapeutic population of TILs obtained from step (e);
(g) transferring the harvested TIL population from step (e) to an infusion bag, wherein the transfer from step (e) to (f) occurs without opening the system; and
(h) wherein one or more of steps (a) to (g) are performed in a closed, sterile system.
   In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing at least a portion of the third population of TILs using electroporation of transcription activator-like effector nuclease-encoding nucleic acids in cytoporation medium to obtain a fourth population of TILs, wherein the gene-editing effects expression of at least one immunomodulatory composition at the cell surface;
(e) optionally incubating the fourth population of TILs; and
(f) culturing the fourth population of TILs in a second cell culture medium comprising antigen presenting cells (APCs), OKT-3, and IL-2 for about 5-15 days, to produce an expanded number of TILs.
   In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) digesting in an enzyme media the tumor tissue to produce a tumor digest;
(c) culturing the first population of TILs in a first cell culture medium comprising IL-2 for about 3-9 days to produce a second population of TILs;
(d) activating the second population of TILs using anti-CD3 and anti-CD28 beads or antibodies for 1-7 days, to produce a third population of TILs;
(e) gene-editing at least a portion of the third population of TILs using electroporation of transcription activator-like effector nuclease-encoding nucleic acids in cytoporation medium to obtain a fourth population of TILs, wherein the gene-editing effects expression of at least one immunomodulatory composition at the cell surface;
(f) optionally incubating the fourth population of TILs, wherein the incubation is performed at about 30-40° C. with about 5% CO₂; and
(g) culturing the fourth population of TILs in a second cell culture medium comprising antigen presenting cells (APCs), OKT-3, and IL-2 for about 5-15 days, to produce an expanded number of TILs.
In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing the third population of TILs by temporarily disrupting the cell membranes of the third population of TILs to effect transfer of transcription activator-like effector nuclease-encoding nucleic acids into the third population of TILs to obtain a fourth population of TILs, wherein the gene-editing effects expression of at least one immunomodulatory composition at the cell surface;
(e) optionally incubating the fourth population of TILs; and
(f) culturing the fourth population of TILs in a second cell culture medium comprising antigen presenting cells (APCs), OKT-3, and IL-2 for about 5-15 days, to produce an expanded number of TILs. In some embodiments, a microfluidic platform is used to temporarily disrupt the cell membranes of the third population of TILs. In some embodiments, the microfluidic platform is a SQZ vector-free microfluidic platform.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing the third population of TILs by temporarily disrupting the cell membranes of the third population of TILs to effect transfer of transcription activator-like effector nuclease-encoding nucleic acids into the third population of TILs to obtain a fourth population of TILs, wherein the gene-editing effects expression of at least one immunomodulatory composition at the cell surface;
(e) optionally incubating the fourth population of TILs, wherein the incubation is performed at about 30-40° C. with about 5% CO₂; and
(f) culturing the fourth population of TILs in a second cell culture medium comprising antigen presenting cells (APCs), OKT-3, and IL-2 for about 5-15 days, to produce an expanded number of TILs. In some embodiments, a microfluidic platform is used to temporarily disrupt the cell membranes of the third population of TILs. In some embodiments, the microfluidic platform is a SQZ vector-free microfluidic platform.

In some embodiments, the step of culturing the fourth population of TILs is performed by culturing the fourth population of TILs in the second cell culture medium for a first period of about 1-7 days, at the end of the first period the culture is split into a plurality of subcultures, each of the plurality of subcultures is cultured in a third culture medium comprising IL-2 for a second period of about 3-7 days, and at the end of the second period the plurality of subcultures are combined to provide the expanded number of TILs.

According to other embodiments, TALENs may be specifically designed, which allows higher rates of DSB events within the target cell(s) that are able to target a specific selection of genes. See U.S. Patent Publication No. 2013/0315884. The use of such rare cutting endonucleases increases the chances of obtaining double inactivation of target genes in transfected cells, allowing for the production of engineered cells, such as T-cells. Further, additional catalytic domains can be introduced with the TALEN to increase mutagenesis and enhance target gene inactivation. The TALENs described in U.S. Patent Publication No. 2013/0315884 were successfully used to engineer T-cells to make them suitable for immunotherapy. TALENs may also be used to inactivate various immune checkpoint genes in T-cells, including the inactivation of at least two genes in a single T-cell. See U.S. Patent Publication No. 2016/0120906. Additionally, TALENs may be used to inactivate genes encoding targets for immunosuppressive agents and T-cell receptors, as disclosed in U.S. Patent Publication No. 2018/0021379, which is incorporated by reference herein. Further, TALENs may be used to inhibit the expression of beta 2-microglobulin (B2M) and/or class II major histocompatibility complex transactivator (CIITA), as disclosed in U.S. Patent Publication No. 2019/0010514, which is incorporated by reference herein.

Non-limiting examples of genes that may be silenced or inhibited by permanently gene-editing TILs via a TALE method include PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

Non-limiting examples of TALE-nucleases targeting the PD-1 gene are provided in the following table. In these examples, the targeted genomic sequences contain two 17-base pair (bp) long sequences (referred to as half targets, shown in upper case letters) separated by a 15-bp spacer (shown in lower case letters). Each half target is recognized by repeats of half TALE-nucleases listed in Table 11. Thus, according to particular embodiments, TALE-nucleases according to the invention recognize and cleave the target sequence selected from the group consisting of: SEQ ID NO: 286 and SEQ ID NO: 287. TALEN sequences and gene-editing methods are also described in Gautron, discussed above.

TABLE 11
TALEN PD-1 Sequences.
TALEN PD-1 No. 1 Sequences

| | |
|---|---|
| Target PD-1 Sequence | TTCTCCCCAGCCCTGCTcgtggtgaccgaaggGGACAACGCCACC |
| (SEQ ID NO: 286) | TTCA |
| Repeat PD-1-left | LTPQQVVAIASNGGGKQALETVQRLLPVLCQAHGLTPEQVVAIAS |
| (SEQ ID NO: 288) | HDGGKQALETVQRLLPVLCQAHGLTPQQVVAIASNGGGKQALETV |
| | QRLLPVLCQAHGLTPEQVVAIASHDGGKQALETVQRLLPVLCQAH |
| | GLTPEQVVAIASHDGGKQALETVQRLLPVLCQAHGLTPEQVVAIA |
| | SHDGGKQALETVQRLLPVLCQAHGLTPEQVVAIASHDGGKQALET |
| | VQRLLPVLCQAHGLTPEQVVAIASNIGGKQALETVQALLPVLCQA |
| | HGLTPQQVVAIASNNGGKQALETVQRLLPVLCQAHGLTPEQVVAI |
| | ASHDGGKQALETVQRLLPVLCQAHGLTPEQVVAIASHDGGKQALE |
| | TVQRLLPVLCQAHGLTPEQVVAIASHDGGKQALETVQRLLPVLCQ |
| | AHGLTPQQVVAIASNGGGKQALETVQRLLPVLCQAHGLTPQQVVA |
| | IASNNGGKQALETVQRLLPVLCQAHGLTPEQVVAIASHDGGKQAL |
| | ETVQRLLPVLCQAHGLTPQQVVAIASNGGGRPALE |
| Repeat PD-1-right | LTPQQVVAIASNNGGKQALETVQRLLPVLCQAHGLTPEQVVAIAS |
| (SEQ ID NO: 289) | NIGGKQALETVQALLPVLCQAHGLTPEQVVAIASNIGGKQALETV |
| | QALLPVLCQAHGLTPQQVVAIASNNGGKQALETVQRLLPVLCQAH |
| | GLTPQQVVAIASNNGGKQALETVQRLLPVLCQAHGLTPQQVVAIA |
| | SNGGGKQALETVQRLLPVLCQAHGLTPQQVVAIASNNGGKQALET |
| | VQRLLPVLCQAHGLTPQQVVAIASNNGGKQALETVQRLLPVLCQA |
| | HGLTPEQVVAIASNGGKQALETVQRLLPVLCQAHGLTPQQVVAIA |
| | SNNGGKQALETVQRLLPVLCQAHGLTPQQVVAIASNGGGKQALET |
| | VQRLLPVLCQAHGLTPQQVVAIASNGGGKQALETVQRLLPVLCQA |
| | HGLTPQQVVAIASNNGGKQALETVQRLLPVLCQAHGLTPQQVVAI |
| | ASNGGGKQALETVQRLLPVLCQAHGLTPEQVVAIASHDGGKQALE |
| | TVQRLLPVLCQAHGLTPQQVVAIASNGGGRPALE |
| PD-1-left TALEN | ATGGGCGATCCTAAAAAGAAACGTAAGGTCATCGATTACCCATAC |
| (SEQ ID NO: 290) | GATGTTCCAGATTACGCTATCGATATCGCCGATCTACGCACGCTC |
| | GGCTACAGCCAGCAGCAACAGGAGAAGATCAAACCGAAGGTTCGT |
| | TCGACAGTGGCGCAGCACCACGAGGCACTGGTCGGCCACGGGTTT |
| | ACACACGCGCACATCGTTGCGTTAAGCCAACACCCGGCAGCGTTA |
| | GGGACCGTCGCTGTCAAGTATCAGGACATGATCGCAGCGTTGCCA |
| | GAGGCGACACACGAAGCGATCGTTGGCGTCGGCAAACAGTGGTCC |
| | GGCGCACGCGCTCTGGAGGCCTTGCTCACGGTGGCGGGAGAGTTG |
| | AGAGGTCCACCGTTACAGTTGGACACAGGCCAACTTCTCAAGATT |
| | GCAAAACGTGGCGGCGTGACCGCAGTGGAGGCAGTGCATGCATGG |
| | CGCAATGCACTGACGGGTGCCCCGCTCAACTTGACCCCCCAGCAG |
| | GTGGTGGCCATCGCCAGCAATGGCGGTGGCAAGCAGGCGCTGGAG |
| | ACGGTCCAGCGGCTGTTGCCGGTGCTGTGCCAGGCCCACGGCTTG |
| | ACCCCGGAGCAGGTGGTGGCCATCGCCAGCCACGATGGCGGCAAG |
| | CAGGCGCTGGAGACGGTCCAGCGGCTGTTGCCGGTGCTGTGCCAG |
| | GCCCACGGCTTGACCCCCCAGCAGGTGGTGGCCATCGCCAGCAAT |
| | GGCGGTGGCAAGCAGGCGCTGGAGACGGTCCAGCGGCTGTTGCCG |
| | GTGCTGTGCCAGGCCCACGGCTTGACCCCGGAGCAGGTGGTGGCC |
| | ATCGCCAGCCACGATGGCGGCAAGCAGGCGCTGGAGACGGTCCAG |
| | CGGCTGTTGCCGGTGCTGTGCCAGGCCCACGGCTTGACCCCGGAG |
| | CAGGTGGTGGCCATCGCCAGCCACGATGGCGGCAAGCAGGCGCTG |
| | GAGACGGTCCAGCGGCTGTTGCCGGTGCTGTGCCAGGCCCACGGC |
| | TTGACCCCGGAGCAGGTGGTGGCCATCGCCAGCCACGATGGCGGC |
| | AAGCAGGCGCTGGAGACGGTCCAGCGGCTGTTGCCGGTGCTGTGC |
| | CAGGCCCACGGCTTGACCCCGGAGCAGGTGGTGGCCATCGCCAGC |
| | CACGATGGCGGCAAGCAGGCGCTGGAGACGGTCCAGCGGCTGTTG |
| | CCGGTGCTGTGCCAGGCCCACGGCTTGACCCCGGAGCAGGTGGTG |
| | GCCATCGCCAGCAATATTGGTGGCAAGCAGGCGCTGGAGACGGTG |
| | CAGGCGCTGTTGCCGGTGCTGTGCCAGGCCCACGGCTTGACCCCC |
| | CAGCAGGTGGTGGCCATCGCCAGCAATAATGGTGGCAAGCAGGCG |
| | CTGGAGACGGTCCAGCGGCTGTTGCCGGTGCTGTGCCAGGCCCAC |
| | GGCTTGACCCCGGAGCAGGTGGTGGCCATCGCCAGCCACGATGGC |
| | GGCAAGCAGGCGCTGGAGACGGTCCAGCGGCTGTTGCCGGTGCTG |
| | TGCCAGGCCCACGGCTTGACCCCGGAGCAGGTGGTGGCCATCGCC |
| | AGCCACGATGGCGGCAAGCAGGCGCTGGAGACGGTCCAGCGGCTG |
| | TTGCCGGTGCTGTGCCAGGCCCACGGCTTGACCCCGGAGCAGGTG |
| | GTGGCCATCGCCAGCCACGATGGCGGCAAGCAGGCGCTGGAGACG |
| | GTCCAGCGGCTGTTGCCGGTGCTGTGCCAGGCCCACGGCTTGACC |
| | CCCCAGCAGGTGGTGGCCATCGCCAGCAATGGCGGTGGCAAGCAG |
| | GCGCTGGAGACGGTCCAGCGGCTGTTGCCGGTGCTGTGCCAGGCC |
| | CACGGCTTGACCCCCCAGCAGGTGGTGGCCATCGCCAGCAATAAT |
| | GGTGGCAAGCAGGCGCTGGAGACGGTCCAGCGGCTGTTGCCGGTG |
| | CTGTGCCAGGCCCACGGCTTGACCCCGGAGCAGGTGGTGGCCATC |
| | GCCAGCCACGATGGCGGCAAGCAGGCGCTGGAGACGGTCCAGCGG |
| | CTGTTGCCGGTGCTGTGCCAGGCCCACGGCTTGACCCCTCAGCAG |
| | GTGGTGGCCATCGCCAGCAATGGCGGCGGCAGGCCGGCGCTGGAG |
| | AGCATTGTTGCCCAGTTATCTCGCCCTGATCCGGCGTTGGCCGCG |
| | TTGACCAACGACCACCTCGTCGCCTTGGCCTGCCTCGGCGGGCGT |
| | CCTGCGCTGGATGCAGTGAAAAAGGGATTGGGGGATCCTATCAGC |
| | CGTTCCCAGCTGGTGAAGTCCGAGCTGGAGGAGAAGAAATCCGAG |
| | TTGAGGCACAAGCTGAAGTACGTGCCCCACGAGTACATCGAGCTG |
| | ATCGAGATCGCCCGGAACAGCACCCAGGACCGTATCCTGGAGATG |
| | AAGGTGATGGAGTTCTTCATGAAGGTGTACGGCTACAGGGGCAAG |
| | CACCTGGGCGGCTCCAGGAAGCCCGACGGCGCCATCTACACCGTG |
| | GGCTCCCCCATCGACTACGGCGTGATCGTGGACACCAAGGCCTAC |
| | TCCGGCGGCTACAACCTGCCCATCGGCCAGGCCGACGAAATGCAG |
| | AGGTACGTGGAGGAGAACCAGACCAGGAACAAGCACATCAACCCC |
| | AACGAGTGGTGGAAGGTGTACCCCTCCAGCGTGACCGAGTTCAAG |
| | TTCCTGTTCGTGTCCGGCCACTTCAAGGGCAACTACAAGGCCCAG |
| | CTGACCAGGCTGAACCACATCACCAACTGCAACGGCGCCGTGCTG |
| | TCCGTGGAGGAGCTCCTGATCGGCGGCGAGATGATCAAGGCCGGC |
| | ACCCTGACCCTGGAGGAGGTGAGGAGGAAGTTCAACAACGGCGAG |
| | ATCAACTTCGCGGCCGACTGATAA |
| PD-1-right TALEN | ATGGGCGATCCTAAAAAGAAACGTAAGGTCATCGATAAGGAGACC |
| (SEQ ID NO: 291) | GCCGCTGCCAAGTTCGAGAGACAGCACATGGACAGCATCGATATC |
| | GCCGATCTACGCACGCTCGGCTACAGCCAGCAGCAACAGGAGAAG |
| | ATCAAACCGAAGGTTCGTTCGACAGTGGCGCAGCACCACGAGGCA |
| | CTGGTCGGCCACGGGTTTACACACGCGCACATCGTTGCGTTAAGC |
| | CAACACCCGGCAGCGTTAGGGACCGTCGCTGTCAAGTATCAGGAC |
| | ATGATCGCAGCGTTGCCAGAGGCGACACACGAAGCGATCGTTGGC |
| | GTCGGCAAACAGTGGTCCGGCGCACGCGCTCTGGAGGCCTTGCTC |
| | ACGGTGGCGGGAGAGTTGAGAGGTCCACCGTTACAGTTGGACACA |
| | GGCCAACTTCTCAAGATTGCAAAACGTGGCGGCGTGACCGCAGTG |
| | GAGGCAGTGCATGCATGGCGCAATGCACTGACGGGTGCCCCGCTC |
| | AACTTGACCCCCCAGCAAGTCGTCGCAATCGCCAGCAATAACGGA |
| | GGGAAGCAAGCCCTCGAAACCGTGCAGCGGTTGCTTCCTGTGCTC |
| | TGCCAGGCCCACGGCCTTACCCCTGAGCAGGTGGTGGCCATCGCA |
| | AGTAACATTGGAGGAAAGCAAGCCTTGGAGACAGTGCAGGCCCTG |
| | TTGCCCGTGCTGTGCCAGGCACACGGCCTCACACCAGAGCAGGTC |
| | GTGGCCATTGCCTCCAACATCGGGGGGAAACAGGCTCTGGAGACC |
| | GTCCAGGCCCTGCTGCCCGTCCTCTGTCAAGCTCACGGCCTGACT |
| | CCCCAACAAGTGGTCGCCATCGCCTCTAATAACGGCGGGAAGCAG |
| | GCACTGGAAACAGTGCAGAGACTGCTCCCTGTGCTTTGCCAAGCT |
| | CATGGGTTGACCCCCCAACAGGTCGTCGCTATTGCCTCAAACAAC |
| | GGGGGCAAGCAGGCCCTTGAGACTGTGCAGAGGCTGTTGCCAGTG |
| | CTGTGTCAGGCTCACGGGCTCACTCCACAACAGGTGGTCGCAATT |
| | GCCAGCAACGGCGGCGGAAAGCAAGCTCTTGAAACCGTGCAACGC |
| | CTCCTGCCCGTGCTCTGTCAGGCTCATGGCCTGACACCACAACAA |
| | GTCGTGGCCATCGCCAGTAATAATGGCGGGAAACAGGCTCTTGAG |
| | ACCGTCCAGAGGCTGCTCCCAGTGCTCTGCCAGGCACACGGGCTG |
| | ACCCCCCAGCAGGTGGTGGCTATCGCCAGCAATAATGGGGGCAAG |
| | CAGGCCCTGGAAACAGTCCAGCGCCTGCTGCCAGTGCTTTGCCAG |
| | GCTCACGGGCTCACTCCCGAACAGGTCGTGGCAATCGCCTCCAAC |
| | GGAGGGAAGCAGGCTCTGGAGACCGTGCAGAGACTGCTGCCCGTC |
| | TTGTGCCAGGCCCACGGACTCACACCTCAGCAGGTCGTCGCCATT |
| | GCCTCTAACAACGGGGGCAAACAAGCCCTGGAGACAGTGCAGCGG |
| | CTGTTGCCTGTGTTGTGCCAAGCCCACGGCTTGACTCCTCAACAA |
| | GTGGTCGCCATCGCCTCAAATGGCGGCGGAAAACAAGCTCTGGAG |
| | ACAGTGCAGAGGTTGCTGCCCGTCCTCTGCCAAGCCCACGGCCTG |
| | ACTCCCCAACAGGTCGTCGCCATTGCCAGCAACGGCGGAGGAAAG |
| | CAGGCTCTCGAAACTGTGCAGCGGCTGCTTCCTGTGCTGTGTCAG |
| | GCTCATGGGCTGACCCCCCAGCAAGTGGTGGCTATTGCCTCTAAC |
| | AATGGAGGCAAGCAAGCCCTTGAGACAGTCCAGAGGCTGTTGCCA |
| | GTGCTGTGCCAGGCCCACGGGCTCACACCCCAGCAGGTGGTCGCC |
| | ATCGCCAGTAACGGCGGGGGCAAACAGGCATTGGAAACCGTCCAG |
| | CGCCTGCTTCCAGTGCTCTGCCAGGCACACGGACTGACACCCGAA |
| | CAGGTGGTGGCCATTGCATCCCATGATGGGGGCAAGCAGGCCCTG |
| | GAGACCGTGCAGAGACTCCTGCCAGTGTTGTGCCAAGCTCACGGC |
| | CTCACCCCTCAGCAAGTCGTGGCCATCGCCTCAAACGGGGGGGGC |
| | CGGCCTGCACTGGAGAGCATTGTTGCCCAGTTATCTCGCCCTGAT |
| | CCGGCGTTGGCCGCGTTGACCAACGACCACCTCGTCGCCTTGGCC |
| | TGCCTCGGCGGGCGTCCTGCGCTGGATGCAGTGAAAAAGGGATTG |
| | GGGGATCCTATCAGCCGTTCCCAGCTGGTGAAGTCCGAGCTGGAG |
| | GAGAAGAAATCCGAGTTGAGGCACAAGCTGAAGTACGTGCCCCAC |
| | GAGTACATCGAGCTGATCGAGATCGCCCGGAACAGCACCCAGGAC |
| | CGTATCCTGGAGATGAAGGTGATGGAGTTCTTCATGAAGGTGTAC |
| | GGCTACAGGGGCAAGCACCTGGGCGGCTCCAGGAAGCCCGACGGC |
| | GCCATCTACACCGTGGGCTCCCCCATCGACTACGGCGTGATCGTG |
| | GACACCAAGGCCTACTCCGGCGGCTACAACCTGCCCATCGGCCAG |
| | GCCGACGAAATGCAGAGGTACGTGGAGGAGAACCAGACCAGGAAC |
| | AAGCACATCAACCCCAACGAGTGGTGGAAGGTGTACCCCTCCAGC |
| | GTGACCGAGTTCAAGTTCCTGTTCGTGTCCGGCCACTTCAAGGGC |
| | AACTACAAGGCCCAGCTGACCAGGCTGAACCACATCACCAACTGC |
| | AACGGCGCCGTGCTGTCCGTGGAGGAGCTCCTGATCGGCGGCGAG |
| | ATGATCAAGGCCGGCACCCTGACCCTGGAGGAGGTGAGGAGGAAG |
| | TTCAACAACGGCGAGATCAACTTCGCGGCCGACTGATAA |

TALEN PD-1 No. 2 Sequences

| | |
|---|---|
| Target PD-1 Sequence | TACCTCTGTGGGGCCATctccctggcccccaaGGCGCAGATCAAA |
| (SEQ ID NO: 287) | GAGA |
| Repeat PD-1-left | LTPEQVVAIASNIGGKQALETVQALLPVLCQAHGLTPEQVVAIAS |
| (SEQ ID NO: 292) | HDGGKQALETVQRLLPVLCQAHGLTPEQVVAIASHDGGKQALETV |
| | QRLLPVLCQAHGLTPQQVVAIASNGGGKQALETVQRLLPVLCQAH |
| | GLTPEQVVAIASHDGGKQALETVQRLLPVLCQAHGLTPQQVVAIA |
| | SNGGGKQALETVQRLLPVLCQAHGLTPQQVVAIASNNGGKQALET |
| | VQRLLPVLCQAHGLTPQQVVAIASNGGGKQALETVQRLLPVLCQA |
| | HGLTPQQVVAIASNNGGKQALETVQRLLPVLCQAHGLTPQQVVAI |
| | ASNNGGKQALETVQRLLPVLCQAHGLTPQQVVAIASNNGGKQALE |
| | TVQRLLPVLCQAHGLTPQQVVAIASNNGGKQALETVQRLLPVLCQ |
| | AHGLTPEQVVAIASHDGGKQALETVQRLLPVLCQAHGLTPEQVVA |
| | IASHDGGKQALETVQRLLPVLCQAHGLTPEQVVAIASNIGGKQAL |
| | ETVQALLPVLCQAHGLTPQQVVAIASNGGGRPALE |
| Repeat PD-1-right | LTPEQVVAIASHDGGKQALETVQRLLPVLCQAHGLTPQQVVAIAS |
| (SEQ ID NO: 293) | NGGGKQALETVQRLLPVLCQAHGLTPEQVVAIASHDGGKQALETV |
| | QRLLPVLCQAHGLTPQQVVAIASNGGGKQALETVQRLLPVLCQAH |
| | GLTPQQVVAIASNGGGKQALETVQRLLPVLCQAHGLTPQQVVAIA |
| | SNGGGKQALETVQRLLPVLCQAHGLTPQQVVAIASNNGGKQALET |
| | VQRLLPVLCQAHGLTPEQVVAIASNIGGKQALETVQALLPVLCQA |
| | HGLTPQQVVAIASNGGGKQALETVQRLLPVLCQAHGLTPEQVVAI |
| | ASHDGGKQALETVQRLLPVLCQAHGLTPQQVVAIASNGGGKQALE |
| | TVQRLLPVLCQAHGLTPQQVVAIASNNGGKQALETVQRLLPVLCQ |
| | AHGLTPEQVVAIASNGGKQALETVQRLLPVLCQAHGLTPQQVVAI |
| | ASNNGGKQALETVQRLLPVLCQAHGLTPEQVVAIASHDGGKQALE |
| | TVQRLLPVLCQAHGLTPQQVVAIASNGGGRPALE |
| PD-1-left TALEN | ATGGGCGATCCTAAAAAGAAACGTAAGGTCATCGATTACCCATAC |
| (SEQ ID NO: 294) | GATGTTCCAGATTACGCTATCGATATCGCCGATCTACGCACGCTC |
| | GGCTACAGCCAGCAGCAACAGGAGAAGATCAAACCGAAGGTTCGT |
| | TCGACAGTGGCGCAGCACCACGAGGCACTGGTCGGCCACGGGTTT |
| | ACACACGCGCACATCGTTGCGTTAAGCCAACACCCGGCAGCGTTA |
| | GGGACCGTCGCTGTCAAGTATCAGGACATGATCGCAGCGTTGCCA |
| | GAGGCGACACACGAAGCGATCGTTGGCGTCGGCAAACAGTGGTCC |
| | GGCGCACGCGCTCTGGAGGCCTTGCTCACGGTGGCGGGAGAGTTG |
| | AGAGGTCCACCGTTACAGTTGGACACAGGCCAACTTCTCAAGATT |
| | GCAAAACGTGGCGGCGTGACCGCAGTGGAGGCAGTGCATGCATGG |
| | CGCAATGCACTGACGGGTGCCCCGCTCAACTTGACCCCGGAGCAG |
| | GTGGTGGCCATCGCCAGCAATATTGGTGGCAAGCAGGCGCTGGAG |
| | ACGGTGCAGGCGCTGTTGCCGGTGCTGTGCCAGGCCCACGGCTTG |
| | ACCCCGGAGCAGGTGGTGGCCATCGCCAGCCACGATGGCGGCAAG |
| | CAGGCGCTGGAGACGGTCCAGCGGCTGTTGCCGGTGCTGTGCCAG |
| | GCCCACGGCTTGACCCCGGAGCAGGTGGTGGCCATCGCCAGCCAC |
| | GATGGCGGCAAGCAGGCGCTGGAGACGGTCCAGCGGCTGTTGCCG |
| | GTGCTGTGCCAGGCCCACGGCTTGACCCCCCAGCAGGTGGTGGCC |
| | ATCGCCAGCAATGGCGGTGGCAAGCAGGCGCTGGAGACGGTCCAG |
| | CGGCTGTTGCCGGTGCTGTGCCAGGCCCACGGCTTGACCCCGGAG |
| | CAGGTGGTGGCCATCGCCAGCCACGATGGCGGCAAGCAGGCGCTG |
| | GAGACGGTCCAGCGGCTGTTGCCGGTGCTGTGCCAGGCCCACGGC |
| | TTGACCCCCCAGCAGGTGGTGGCCATCGCCAGCAATGGCGGTGGC |
| | AAGCAGGCGCTGGAGACGGTCCAGCGGCTGTTGCCGGTGCTGTGC |
| | CAGGCCCACGGCTTGACCCCCCAGCAGGTGGTGGCCATCGCCAGC |
| | AATAATGGTGGCAAGCAGGCGCTGGAGACGGTCCAGCGGCTGTTG |
| | CCGGTGCTGTGCCAGGCCCACGGCTTGACCCCCCAGCAGGTGGTG |
| | GCCATCGCCAGCAATGGCGGTGGCAAGCAGGCGCTGGAGACGGTC |
| | CAGCGGCTGTTGCCGGTGCTGTGCCAGGCCCACGGCTTGACCCCC |
| | CAGCAGGTGGTGGCCATCGCCAGCAATAATGGTGGCAAGCAGGCG |
| | CTGGAGACGGTCCAGCGGCTGTTGCCGGTGCTGTGCCAGGCCCAC |
| | GGCTTGACCCCCCAGCAGGTGGTGGCCATCGCCAGCAATAATGGT |
| | GGCAAGCAGGCGCTGGAGACGGTCCAGCGGCTGTTGCCGGTGCTG |
| | TGCCAGGCCCACGGCTTGACCCCCCAGCAGGTGGTGGCCATCGCC |
| | AGCAATAATGGTGGCAAGCAGGCGCTGGAGACGGTCCAGCGGCTG |
| | TTGCCGGTGCTGTGCCAGGCCCACGGCTTGACCCCCCAGCAGGTG |
| | GTGGCCATCGCCAGCAATAATGGTGGCAAGCAGGCGCTGGAGACG |
| | GTCCAGCGGCTGTTGCCGGTGCTGTGCCAGGCCCACGGCTTGACC |
| | CCGGAGCAGGTGGTGGCCATCGCCAGCCACGATGGCGGCAAGCAG |
| | GCGCTGGAGACGGTCCAGCGGCTGTTGCCGGTGCTGTGCCAGGCC |
| | CACGGCTTGACCCCGGAGCAGGTGGTGGCCATCGCCAGCCACGAT |
| | GGCGGCAAGCAGGCGCTGGAGACGGTCCAGCGGCTGTTGCCGGTG |
| | CTGTGCCAGGCCCACGGCTTGACCCCGGAGCAGGTGGTGGCCATC |
| | GCCAGCAATATTGGTGGCAAGCAGGCGCTGGAGACGGTGCAGGCG |
| | CTGTTGCCGGTGCTGTGCCAGGCCCACGGCTTGACCCCTCAGCAG |
| | GTGGTGGCCATCGCCAGCAATGGCGGCGGCAGGCCGGCGCTGGAG |
| | AGCATTGTTGCCCAGTTATCTCGCCCTGATCCGGCGTTGGCCGCG |
| | TTGACCAACGACCACCTCGTCGCCTTGGCCTGCCTCGGCGGGCGT |
| | CCTGCGCTGGATGCAGTGAAAAAGGGATTGGGGGATCCTATCAGC |
| | CGTTCCCAGCTGGTGAAGTCCGAGCTGGAGGAGAAGAAATCCGAG |
| | TTGAGGCACAAGCTGAAGTACGTGCCCCACGAGTACATCGAGCTG |
| | ATCGAGATCGCCCGGAACAGCACCCAGGACCGTATCCTGGAGATG |
| | AAGGTGATGGAGTTCTTCATGAAGGTGTACGGCTACAGGGGCAAG |
| | CACCTGGGCGGCTCCAGGAAGCCCGACGGCGCCATCTACACCGTG |
| | GGCTCCCCCATCGACTACGGCGTGATCGTGGACACCAAGGCCTAC |
| | TCCGGCGGCTACAACCTGCCCATCGGCCAGGCCGACGAAATGCAG |
| | AGGTACGTGGAGGAGAACCAGACCAGGAACAAGCACATCAACCCC |
| | AACGAGTGGTGGAAGGTGTACCCCTCCAGCGTGACCGAGTTCAAG |
| | TTCCTGTTCGTGTCCGGCCACTTCAAGGGCAACTACAAGGCCCAG |
| | CTGACCAGGCTGAACCACATCACCAACTGCAACGGCGCCGTGCTG |
| | TCCGTGGAGGAGCTCCTGATCGGCGGCGAGATGATCAAGGCCGGC |
| | ACCCTGACCCTGGAGGAGGTGAGGAGGAAGTTCAACAACGGCGAG |
| | ATCAACTTCGCGGCCGACTGATAA |
| PD-1-right TALEN | ATGGGCGATCCTAAAAAGAAACGTAAGGTCATCGATAAGGAGACC |
| (SEQ ID NO: 295) | GCCGCTGCCAAGTTCGAGAGACAGCACATGGACAGCATCGATATC |
| | GCCGATCTACGCACGCTCGGCTACAGCCAGCAGCAACAGGAGAAG |
| | ATCAAACCGAAGGTTCGTTCGACAGTGGCGCAGCACCACGAGGCA |
| | CTGGTCGGCCACGGGTTTACACACGCGCACATCGTTGCGTTAAGC |
| | CAACACCCGGCAGCGTTAGGGACCGTCGCTGTCAAGTATCAGGAC |
| | ATGATCGCAGCGTTGCCAGAGGCGACACACGAAGCGATCGTTGGC |
| | GTCGGCAAACAGTGGTCCGGCGCACGCGCTCTGGAGGCCTTGCTC |
| | ACGGTGGCGGGAGAGTTGAGAGGTCCACCGTTACAGTTGGACACA |
| | GGCCAACTTCTCAAGATTGCAAAACGTGGCGGCGTGACCGCAGTG |
| | GAGGCAGTGCATGCATGGCGCAATGCACTGACGGGTGCCCCGCTC |
| | AACTTGACCCCCGAGCAAGTCGTCGCAATCGCCAGCCATGATGGA |
| | GGGAAGCAAGCCCTCGAAACCGTGCAGCGGTTGCTTCCTGTGCTC |
| | TGCCAGGCCCACGGCCTTACCCCTCAGCAGGTGGTGGCCATCGCA |
| | AGTAACGGAGGAGGAAAGCAAGCCTTGGAGACAGTGCAGCGCCTG |
| | TTGCCCGTGCTGTGCCAGGCACACGGCCTCACACCAGAGCAGGTC |
| | GTGGCCATTGCCTCCCATGACGGGGGGAAACAGGCTCTGGAGACC |
| | GTCCAGAGGCTGCTGCCCGTCCTCTGTCAAGCTCACGGCCTGACT |
| | CCCCAACAAGTGGTCGCCATCGCCTCTAATGGCGGCGGGAAGCAG |
| | GCACTGGAAACAGTGCAGAGACTGCTCCCTGTGCTTTGCCAAGCT |
| | CATGGGTTGACCCCCCAACAGGTCGTCGCTATTGCCTCAAACGGG |
| | GGGGGCAAGCAGGCCCTTGAGACTGTGCAGAGGCTGTTGCCAGTG |
| | CTGTGTCAGGCTCACGGGCTCACTCCACAACAGGTGGTCGCAATT |
| | GCCAGCAACGGCGGCGGAAAGCAAGCTCTTGAAACCGTGCAACGC |
| | CTCCTGCCCGTGCTCTGTCAGGCTCATGGCCTGACACCACAACAA |
| | GTCGTGGCCATCGCCAGTAATAATGGCGGGAAACAGGCTCTTGAG |
| | ACCGTCCAGAGGCTGCTCCCAGTGCTCTGCCAGGCACACGGGCTG |
| | ACCCCCGAGCAGGTGGTGGCTATCGCCAGCAATATTGGGGGCAAG |
| | CAGGCCCTGGAAACAGTCCAGGCCCTGCTGCCAGTGCTTTGCCAG |
| | GCTCACGGGCTCACTCCCCAGCAGGTCGTGGCAATCGCCTCCAAC |
| | GGCGGAGGGAAGCAGGCTCTGGAGACCGTGCAGAGACTGCTGCCC |
| | GTCTTGTGCCAGGCCCACGGACTCACACCTGAACAGGTCGTCGCC |
| | ATTGCCTCTCACGATGGGGGCAAACAAGCCCTGGAGACAGTGCAG |
| | CGGCTGTTGCCTGTGTTGTGCCAAGCCCACGGCTTGACTCCTCAA |
| | CAAGTGGTCGCCATCGCCTCAAATGGCGGCGGAAAACAAGCTCTG |
| | GAGACAGTGCAGAGGTTGCTGCCCGTCCTCTGCCAAGCCCACGGC |
| | CTGACTCCCCAACAGGTCGTCGCCATTGCCAGCAACAACGGAGGA |
| | AAGCAGGCTCTCGAAACTGTGCAGCGGCTGCTTCCTGTGCTGTGT |
| | CAGGCTCATGGGCTGACCCCCGAGCAAGTGGTGGCTATTGCCTCT |
| | AATGGAGGCAAGCAAGCCCTTGAGACAGTCCAGAGGCTGTTGCCA |
| | GTGCTGTGCCAGGCCCACGGGCTCACACCCCAGCAGGTGGTCGCC |
| | ATCGCCAGTAACAACGGGGGCAAACAGGCATTGGAAACCGTCCAG |
| | CGCCTGCTTCCAGTGCTCTGCCAGGCACACGGACTGACACCCGAA |
| | CAGGTGGTGGCCATTGCATCCCATGATGGGGGCAAGCAGGCCCTG |
| | GAGACCGTGCAGAGACTCCTGCCAGTGTTGTGCCAAGCTCACGGC |
| | CTCACCCCTCAGCAAGTCGTGGCCATCGCCTCAAACGGGGGGGGC |
| | CGGCCTGCACTGGAGAGCATTGTTGCCCAGTTATCTCGCCCTGAT |
| | CCGGCGTTGGCCGCGTTGACCAACGACCACCTCGTCGCCTTGGCC |
| | TGCCTCGGCGGGCGTCCTGCGCTGGATGCAGTGAAAAAGGGATTG |
| | GGGGATCCTATCAGCCGTTCCCAGCTGGTGAAGTCCGAGCTGGAG |
| | GAGAAGAAATCCGAGTTGAGGCACAAGCTGAAGTACGTGCCCCAC |
| | GAGTACATCGAGCTGATCGAGATCGCCCGGAACAGCACCCAGGAC |
| | CGTATCCTGGAGATGAAGGTGATGGAGTTCTTCATGAAGGTGTAC |
| | GGCTACAGGGGCAAGCACCTGGGCGGCTCCAGGAAGCCCGACGGC |
| | GCCATCTACACCGTGGGCTCCCCCATCGACTACGGCGTGATCGTG |
| | GACACCAAGGCCTACTCCGGCGGCTACAACCTGCCCATCGGCCAG |
| | GCCGACGAAATGCAGAGGTACGTGGAGGAGAACCAGACCAGGAAC |
| | AAGCACATCAACCCCAACGAGTGGTGGAAGGTGTACCCCTCCAGC |
| | GTGACCGAGTTCAAGTTCCTGTTCGTGTCCGGCCACTTCAAGGGC |
| | AACTACAAGGCCCAGCTGACCAGGCTGAACCACATCACCAACTGC |
| | AACGGCGCCGTGCTGTCCGTGGAGGAGCTCCTGATCGGCGGCGAG |
| | ATGATCAAGGCCGGCACCCTGACCCTGGAGGAGGTGAGGAGGAAG |
| | TTCAACAACGGCGAGATCAACTTCGCGGCCGACTGATAA |

In some embodiments, a method for expanding tumor infiltrating lymphocytes (TILs) into a therapeutic population of TILs comprises the steps of:
(a) activating a first population of TILs obtained from a tumor resected from a patient using CD3 and CD28 activating beads or antibodies for 1 to 5 days;
(b) gene-editing at least a portion of the first population of TILs, wherein the gene-editing comprises using electroporation of transcription activator-like effector nuclease-encoding nucleic acids targeting PD-1 in cytoporation medium to obtain a second population of TILs, and wherein the gene-editing effects expression of at least one immunomodulatory composition at the cell surface, and inhibits expression of PD-1, in the portion of the cells of the second population of TILs;
(c) optionally incubating the second population of TILs, wherein the incubation is performed at about 30-40° C. with about 5% CO₂;
(d) performing a first expansion by culturing the second population of TILs in a cell culture medium comprising IL-2, and optionally OKT-3, to produce a third population of TILs, wherein the first expansion is performed in a closed container providing a first gas-permeable surface area, wherein the first expansion is performed for about 6 to 9 days to obtain the third population of TILs;
(e) performing a second expansion by supplementing the cell culture medium of the third population of TILs with additional IL-2, OKT-3, and antigen presenting cells (APCs), to produce a fourth population of TILs, wherein the second expansion is performed for about 9 to 11 days to obtain the fourth population of TILs, wherein the fourth population of TILs is a therapeutic population of TILs;
(f) harvesting the therapeutic population of TILs obtained from step (e);
(g) transferring the harvested TIL population from step (e) to an infusion bag, wherein the transfer from step (e) to (f) occurs without opening the system; and
(h) wherein one or more of steps (a) to (g) are performed in a closed, sterile system.
   In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing at least a portion of the third population of TILs, wherein the gene-editing comprises using electroporation of transcription activator-like effector nuclease-encoding nucleic acids targeting PD-1 in cytoporation medium to obtain a fourth population of TILs, and wherein the gene-editing effects expression of at least one immunomodulatory composition at the cell surface, and inhibits expression of PD-1, in the portion of the cells of the third population of TILs;
(e) optionally incubating the fourth population of TILs, wherein the incubation is performed at about 30-40° C. with about 5% CO₂; and
(f) culturing the fourth population of TILs in a second cell culture medium comprising antigen presenting cells (APCs), OKT-3, and IL-2 for about 5-15 days, to produce an expanded number of TILs.
   In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing at least a portion of the third population of TILs by temporarily disrupting the cell membranes of the third population of TILs to effect transfer of transcription activator-like effector nuclease-encoding nucleic acids targeting PD-1 into the third population of TILs to obtain a fourth population of TILs, wherein the gene-editing effects expression of at least one immunomodulatory composition at the cell surface, and inhibits expression of PD-1, in the portion of the cells of the third population of TILs;
(e) optionally incubating the fourth population of TILs, wherein the incubation is performed at about 30-40° C. with about 5% CO₂; and
(f) culturing the fourth population of TILs in a second cell culture medium comprising antigen presenting cells (APCs), OKT-3, and IL-2 for about 5-15 days, to produce an expanded number of TILs.
   In some embodiments, a microfluidic platform is used to temporarily disrupt the cell membranes of the third population of TILs. In some embodiments, the microfluidic platform is a SQZ vector-free microfluidic platform. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, a method for expanding tumor infiltrating lymphocytes (TILs) into a therapeutic population of TILs comprises the steps of:
(a) activating a first population of TILs obtained from a tumor resected from a patient using CD3 and CD28 activating beads or antibodies for 1 to 5 days;
(b) gene-editing at least a portion of the first population of TILs, wherein the gene-editing comprises using electroporation of transcription activator-like effector nuclease-encoding nucleic acids targeting SEQ ID NO:149 or SEQ ID NO:150 in cytoporation medium to obtain a second population of TILs, and wherein the gene-editing effects expression of at least one immunomodulatory composition at the cell surface, and inhibits expression of PD-1, in the portion of the cells of the second population of TILs;
(c) optionally incubating the second population of TILs, wherein the incubation is performed at about 30-40° C. with about 5% CO₂;
(d) performing a first expansion by culturing the second population of TILs in a cell culture medium comprising IL-2, and optionally OKT-3, to produce a third population of TILs, wherein the first expansion is performed in a closed container providing a first gas-permeable surface area, wherein the first expansion is performed for about 6 to 9 days to obtain the third population of TILs;
(e) performing a second expansion by supplementing the cell culture medium of the third population of TILs with additional IL-2, OKT-3, and antigen presenting cells (APCs), to produce a fourth population of TILs, wherein the second expansion is performed for about 9 to 11 days to obtain the fourth population of TILs, wherein the fourth population of TILs is a therapeutic population of TILs;
(f) harvesting the therapeutic population of TILs obtained from step (e);
(g) transferring the harvested TIL population from step (e) to an infusion bag, wherein the transfer from step (e) to (f) occurs without opening the system; and
(h) wherein one or more of steps (a) to (g) are performed in a closed, sterile system.
   In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing at least a portion of the third population of TILs, wherein the gene-editing comprises using electroporation of transcription activator-like effector nuclease-encoding nucleic acids targeting SEQ ID NO: 149 or SEQ ID NO:150 in cytoporation medium to obtain a fourth population of TILs, and wherein the gene-editing effects expression of at least one immunomodulatory composition at the cell surface, and inhibits expression of PD-1, in the portion of the cells of the third population of TILs;
(e) optionally incubating the fourth population of TILs, wherein the incubation is performed at about 30-40° C. with about 5% CO₂; and
(f) culturing the fourth population of TILs in a second cell culture medium comprising antigen presenting cells (APCs), OKT-3, and IL-2 for about 5-15 days, to produce an expanded number of TILs.

In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing at least a portion of the third population of TILs by temporarily disrupting the cell membranes of the third population of TILs to effect transfer of transcription activator-like effector nuclease-encoding nucleic acids targeting SEQ ID NO: 149 or SEQ ID NO:150 into the third population of TILs to obtain a fourth population of TILs, wherein the gene-editing effects expression of at least one immunomodulatory composition at the cell surface, and inhibits expression of PD-1, in the portion of the cells of the third population of TILs;
(e) optionally incubating the fourth population of TILs, wherein the incubation is performed at about 30-40° C. with about 5% CO₂; and
(f) culturing the fourth population of TILs in a second cell culture medium comprising antigen presenting cells (APCs), OKT-3, and IL-2 for about 5-15 days, to produce an expanded number of TILs.

In some embodiments, a microfluidic platform is used to temporarily disrupt the cell membranes of the third population of TILs. In some embodiments, the microfluidic platform is a SQZ vector-free microfluidic platform. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, a method for expanding tumor infiltrating lymphocytes (TILs) into a therapeutic population of TILs comprises the steps of:
(a) activating a first population of TILs obtained from a tumor resected from a patient using CD3 and CD28 activating beads or antibodies for 1 to 5 days;
(b) gene-editing at least a portion of the first population of TILs, wherein the gene-editing comprises using electroporation of transcription activator-like effector nuclease-encoding mRNAs according to SEQ ID NO: 157 and SEQ ID NO:158 or SEQ ID NO: 153 and SEQ ID NO:154 in cytoporation medium to obtain a second population of TILs, and wherein the gene-editing effects expression of at least one immunomodulatory composition at the cell surface, and inhibits expression of PD-1, in the portion of the cells of the second population of TILs;
(c) optionally incubating the second population of TILs, wherein the incubation is performed at about 30-40° C. with about 5% CO₂;
(d) performing a first expansion by culturing the second population of TILs in a cell culture medium comprising IL-2, and optionally OKT-3, to produce a third population of TILs, wherein the first expansion is performed in a closed container providing a first gas-permeable surface area, wherein the first expansion is performed for about 6 to 9 days to obtain the third population of TILs;
(e) performing a second expansion by supplementing the cell culture medium of the third population of TILs with additional IL-2, OKT-3, and antigen presenting cells (APCs), to produce a fourth population of TILs, wherein the second expansion is performed for about 9 to 11 days to obtain the fourth population of TILs, wherein the fourth population of TILs is a therapeutic population of TILs;
(f) harvesting the therapeutic population of TILs obtained from step (e);
(g) transferring the harvested TIL population from step (e) to an infusion bag, wherein the transfer from step (e) to (f) occurs without opening the system; and
(h) wherein one or more of steps (a) to (g) are performed in a closed, sterile system.

In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing at least a portion of the third population of TILs, wherein the gene-editing comprises using electroporation of transcription activator-like effector nuclease-encoding mRNAs according to SEQ ID NO:157 and SEQ ID NO:158 or SEQ ID NO: 153 and SEQ ID NO: 154 in cytoporation medium to obtain a fourth population of TILs, and wherein the gene-editing effects expression of at least one immunomodulatory composition at the cell surface, and inhibits expression of PD-1, in the portion of the cells of the third population of TILs;
(e) optionally incubating the fourth population of TILs, wherein the incubation is performed at about 30-40° C. with about 5% CO₂; and
(f) culturing the fourth population of TILs in a second cell culture medium comprising antigen presenting cells (APCs), OKT-3, and IL-2 for about 5-15 days, to produce an expanded number of TILs.
   In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, provided herein is a method for preparing expanded tumor infiltrating lymphocytes (TILs) comprising:
(a) obtaining and/or receiving a first population of TILs from a tumor tissue resected from a subject or patient;
(b) culturing the first population of TILs in a first cell culture medium comprising IL-2 for about 3-9 days to produce a second population of TILs;
(c) activating the second population of TILs using anti-CD3 and anti-CD28 beads or antibodies for 1-7 days, to produce a third population of TILs;
(d) gene-editing at least a portion of the third population of TILs by temporarily disrupting the cell membranes of the third population of TILs to effect transfer of transcription activator-like effector nuclease-encoding mRNAs according to SEQ ID NO:157 and SEQ ID NO:158 or SEQ ID NO: 153 and SEQ ID NO:154 into the third population of TILs to obtain a fourth population of TILs, wherein the gene-editing effects expression of at least one immunomodulatory composition at the cell surface, and inhibits expression of PD-1, in the portion of the cells of the third population of TILs;
(e) optionally incubating the fourth population of TILs, wherein the incubation is performed at about 30-40° C. with about 5% CO₂; and
(f) culturing the fourth population of TILs in a second cell culture medium comprising antigen presenting cells (APCs), OKT-3, and IL-2 for about 5-15 days, to produce an expanded number of TILs.
   In some embodiments, a microfluidic platform is used to temporarily disrupt the cell membranes of the third population of TILs. In some embodiments, the microfluidic platform is a SQZ vector-free microfluidic platform. In some embodiments, the immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

Other non-limiting examples of genes that may be enhanced by permanently gene-editing TILs via a TALE method include CCR2, CCR4, CCR5, CXCR2, CXCR3, CX3CR1, IL-2, IL-4, IL-7, IL-10, IL-15, IL-21, the NOTCH 1/2 intracellular domain (ICD), and/or the NOTCH ligand mDLL1.

Examples of systems, methods, and compositions for altering the expression of a target gene sequence by a TALE method, and which may be used in accordance with embodiments of the present invention, are described in U.S. Pat. No. 8,586,526, which is incorporated by reference herein. These disclosed examples include the use of a non-naturally occurring DNA-binding polypeptide that has two or more TALE-repeat units containing a repeat RVD, an N-cap polypeptide made of residues of a TALE protein, and a C-cap polypeptide made of a fragment of a full length C-terminus region of a TALE protein.

Examples of TALEN designs and design strategies, activity assessments, screening strategies, and methods that can be used to efficiently perform TALEN-mediated gene integration and inactivation, and which may be used in accordance with embodiments of the present invention, are described in Valton, et al., Methods, 2014, 69, 151-170, which is incorporated by reference herein.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, and wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of a TALE nuclease system that effects expression of at least one immunomodulatory composition at the cell surface of and modulates expression of at least one immune checkpoint protein and/or adhesion molecule, in the plurality of cells of the second population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, and wherein the transition from step (c) to step (d) occurs without opening the system;
(e) temporarily disrupting the cell membranes of the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the transfer of the at least one gene editor step comprises the delivery of a TALE nuclease system that effects expression of at least one immunomodulatory composition at the cell surface of and modulates expression of at least one immune checkpoint protein and/or adhesion molecule, in the plurality of cells of the second population of TILs. In some embodiments, a microfluidic platform is used to temporarily disrupt the cell membranes of the third population of TILs. In some embodiments, the microfluidic platform is a SQZ vector-free microfluidic platform. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) sterile electroporating step on the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of a TALE nuclease system that effects expression of at least one immunomodulatory composition at the cell surface of and suppresses the expression of PD-1 and LAG-3 in the plurality of cells of the second population of TILs. In some embodiments, the at least one immunomodulatory composition comprises a cytokine fused to a membrane anchor. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) temporarily disrupting the cell membranes of the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the transfer of the at least one gene editor step comprises the delivery of a TALE nuclease system that effects expression of at least one immunomodulatory composition at the cell surface of and suppresses the expression of PD-1 and LAG-3 in the plurality of cells of the second population of TILs. In some embodiments, a microfluidic platform is used to temporarily disrupt the cell membranes of the third population of TILs. In some embodiments, the microfluidic platform is a SQZ vector-free microfluidic platform. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

### 3. Zinc Finger Methods

A method for expanding TILs into a therapeutic population may be carried out in accordance with any embodiment of the methods described herein (e.g., process 2A) or as described in PCT/US2017/058610, PCT/US2018/012605, or PCT/US2018/012633, wherein the method further comprises gene-editing at least a portion of the TILs by a zinc finger or zinc finger nuclease method. According to particular embodiments, the use of a zinc finger method during the TIL expansion process causes expression of at least one immunomodulatory composition at the cell surface, and optionally causes expression of one or more immune checkpoint genes to be silenced or reduced in at least a portion of the therapeutic population of TILs. Alternatively, the use of a zinc finger method during the TIL expansion process causes expression of at least one immunomodulatory composition at the cell surface, and optionally causes expression of one or more immune checkpoint genes to be enhanced in at least a portion of the therapeutic population of TILs.

An individual zinc finger contains approximately 30 amino acids in a conserved 00a configuration. Several amino acids on the surface of the u-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially; Sangamo Biosciences (Richmond, CA, USA) has developed a propriety platform (CompoZr®) for zinc-finger construction in partnership with Sigma-Aldrich (St. Louis, MO, USA).

Non-limiting examples of genes that may be silenced or inhibited by permanently gene-editing TILs via a zinc finger method include include PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

Non-limiting examples of genes that may be enhanced by permanently gene-editing TILs via a zinc finger method include CCR2, CCR4, CCR5, CXCR2, CXCR3, CX3CR1, IL-2, IL-4, IL-7, IL-10, IL-15, IL-18, IL-21, the NOTCH 1/2 intracellular domain (ICD), and/or the NOTCH ligand mDLL1.

Examples of systems, methods, and compositions for altering the expression of a target gene sequence by a zinc finger method, which may be used in accordance with embodiments of the present invention, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, which are incorporated by reference herein.

Other examples of systems, methods, and compositions for altering the expression of a target gene sequence by a zinc finger method, which may be used in accordance with embodiments of the present invention, are described in Beane, et al., Mol. Therapy, 2015, 23 1380-1390, the disclosure of which is incorporated by reference herein.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of a zinc finger nuclease system that effects expression of at least one immunomodulatory composition at the cell surface of and modulates the expression of at least one immune checkpoint protein and/or adhesion molecule in the plurality of cells of the second population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) temporarily disrupting the cell membranes of the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the transfer of the at least one gene editor step comprises the delivery of a zinc finger nuclease system that effects expression of at least one immunomodulatory composition at the cell surface of and modulates the expression of at least one immune checkpoint protein and/or adhesion molecule in the plurality of cells of the second population of TILs. In some embodiments, a microfluidic platform is used to temporarily disrupt the cell membranes of the third population of TILs. In some embodiments, the microfluidic platform is a SQZ vector-free microfluidic platform. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of a zinc finger nuclease system that effects expression of at least one immunomodulatory composition at the cell surface of and suppresses the expression of PD-1 and LAG-3 in the plurality of cells of the second population of TILs. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

### 4. Cas-CLOVER Methods

A method for expanding TILs into a therapeutic population may be carried out in accordance with any embodiment of the methods described herein (e.g., process 2A) or as described in PCT/US2017/058610, PCT/US2018/012605, or PCT/US2018/012633, wherein the method further comprises gene-editing at least a portion of the TILs by a Cas-CLOVER method. According to particular embodiments, the use of a Cas-CLOVER method during the TIL expansion process causes expression of one or more immune checkpoint genes to be silenced or reduced in at least a portion of the therapeutic population of TILs. Alternatively, the use of a Cas-CLOVER method during the TIL expansion process causes expression of one or more immune checkpoint genes to be enhanced in at least a portion of the therapeutic population of TILs.

Cas-CLOVER is a dimeric, high-fidelity site-specific nuclease (SSN) that consists of a fusion of catalytically dead SpCas9 (dCas9) with the nuclease domain from a Clostridium Clo051 type IIs restriction endonuclease (Madison, et al., “Cas-CLOVER is a novel high-fidelity nuclease for safe and robust generation of T SCM-enriched allogeneic CAR-T cells,” Molecular Therapy—Nucleic Acids, 2022). This yields a nuclease whose activity is predicated upon the dimerization of the Clo051 nuclease domain, enabled by RNA-guided recognition of two adjacent 20-nt target sequences. Unlike a paired nickase approach, e.g., when using the Cas9-D10A mutant, monomeric Cas-CLOVER does not introduce a nick or a DSB. Cas-CLOVER has been shown to have low off-target nuclease activity.

Exemplary Cas-CLOVER systems include those described in WO2019/126578, the contents of which are incorporated herein by reference in their entirety. In some embodiments, the Cas-CLOVER system comprises a fusion protein comprising, consisting essentially of, or consisting of a DNA localization component and an effector molecule.

### DNA Localization Components

In some embodiments, the DNA localization components are capable of binding a specific DNA sequence. In some embodiments, the DNA localization component is selected from, for example, a DNA-binding oligonucleotide, a DNA-binding protein, a DNA binding protein complex, and combinations thereof. Other suitable DNA binding components will be recognized by one of ordinary skill in the art.

In some embodiments, the DNA localization components comprise an oligonucleotide directed to a specific locus or loci in the genome. The oligonucleotide may be selected from DNA, RNA, DNA/RNA hybrids, and combinations thereof.

In some embodiments, the DNA localization components comprise a nucleotide binding protein or protein complex that binds an oligonucleotide when bound to a target DNA. The protein or protein complex may be capable of recognizing a feature selected from RNA-DNA heteroduplexes, R-loops, or combinations thereof. In some embodiments, the DNA localization component comprises a protein or protein complex capable of recognizing an R-loop selected from Cas9, Cascade complex, RecA, RNase H, RNA polymerase, DNA polymerase, or a combination thereof. In some embodiments, the DNA localization component comprises an engineered protein capable of binding to target DNA. In some embodiments, the DNA localization component comprises a protein capable of binding a DNA sequence selected from meganuclease, zinc finger array, transcription activator-like (TAL) array, and combinations thereof. In some embodiments, the DNA localization component comprises a protein that contains a naturally occurring DNA binding domain. In some embodiments, the DNA localization component comprises a bZIP domain, a Helix-loop-helix, a Helix-turn-helix, a HMG-box, a Leucine zipper, a Zinc finger, or a combination thereof. In some embodiments, the DNA localization component comprises an oligonucleotide directed to a specific locus in the genome. Exemplary oligonucleotides include, but are not limited to, DNA, RNA, DNA/RNA hybrids, and any combination thereof. In some embodiments, the DNA localization component comprises a protein or a protein complex capable of recognizing a feature selected from RNA-DNA heteroduplexes, R-loops, and any combination thereof. Exemplary proteins or protein complexes capable of recognizing an R-loop include, but are not limited to, Cas9, Cascade complex, RecA, RNase H, RNA polymerase, DNA polymerase, and any combination thereof. In some embodiments, the protein or protein complex capable of recognizing an R-loop comprises Cas9. In some embodiments, the DNA localization component comprises a protein capable of binding a DNA sequence selected from meganuclease, Zinc Finger array, TAL array, and any combination thereof. In some embodiments, the DNA localization component comprises an oligonucleotide directed to a target location in a genome and a protein capable of binding to a target DNA sequence.

In some embodiments, the DNA localization components comprise, consist essentially of, or consist of, at least one guide RNA (gRNA). In some embodiments, the DNA localization components comprise, consist essentially of, or consist of, two gRNAs, wherein a first gRNA specifically binds to a first strand of a double-stranded DNA target sequence and a second gRNA specifically binds to a second strand of the double-stranded DNA target sequence. Alternatively, in embodiments, DNA localization components comprise, consist essentially of, or consist of, a DNA binding domain of a transcription activator-like effector nuclease (TALEN, also referred to as a TAL protein). In some embodiments DNA localization components comprise, consist essentially of, or consist of, a DNA-binding domain of a TALEN, or TAL protein, derived from Xanthomonas or Ralstonia.

### Effector Molecules

In some embodiments, effector molecules are capable of a predetermined effect at a specific locus in the genome. Exemplary effector molecules, but are not limited to, a transcription factor (activator or repressor), chromatin remodeling factor, nuclease, exonuclease, endonuclease, transposase, methytransferase, demethylase, acetyltransferase, deacetylase, kinase, phosphatase, integrase, recombinase, ligase, topoisomerase, gyrase, helicase, fluorophore, or any combination thereof.

In some embodiments, effector molecules comprise a transposase. In some embodiments, effector molecules comprise a PB transposase (PBase). In some embodiments, effector molecules comprise a nuclease. Non-limiting examples of nucleases include restriction endonucleases, homing endonucleases, S1 nuclease, mung bean nuclease, pancreatic DNase I, micrococcal nuclease, yeast HO endonuclease, or any combination thereof. In certain embodiments, the effector molecule comprises a restriction endonuclease. In certain embodiments, the effector molecule comprises a Type IIS restriction endonuclease. In some embodiments, effector molecules comprise an endonuclease. Non-limiting examples of the endonuclease include AciI, MnlI, AlwI, BbvI, BccI, BceAI, BsmAI, BsmFI, BspCNI, BsrI, BtsCI, HgaI, HphI, HpyAV, MbolI, MylI, PleI, SfaNI, AcuI, BciVI, BfuAI, BmgBI, BmrI, BpmI, BpuEI, BsaI, BseRI, BsgI, BsmI, BspMI, BsrBI, BsrBI, BsrDI, BtgZI, BtsI, EarI, EciI, MmeI, NmeAIII, BbvCI, Bpu10I, BspQI, SapI, BaeI, BsaXI, CspCI, BfiI, MboII, Acc36I and Clo051. In some embodiments, the effector molecule comprises BmrI, BfiI, or Clo051.

In some embodiments, effector molecules comprise, consist essentially of, or consist of, a homodimer or a heterodimer. In some embodiments, effector molecules comprise, consist essentially of, or consist of, a nuclease, optionally an endonuclease. In some embodiments, effector molecules, including those effector molecules comprising a homodimer or a heterodimer, comprise, consist essentially of, or consist of, a Cas9, a Cas9 nuclease domain or a fragment thereof. In some embodiments, the Cas9 is a catalytically inactive or “inactivated” Cas9 (dCas9 (SEQ ID NO: 302 and 303 of WO2019/126578)). In some embodiments, the Cas9 is a catalytically inactive or “inactivated” nuclease domain of Cas9. In some embodiments, the dCas9 is encoded by a shorter sequence that is derived from a full length, catalytically inactivated, Cas9, referred to herein as a “small” dCas9 or dSaCas9 (SEQ ID NO: 23 of WO2019/126578).

In some embodiments of the fusion protein, the effector molecule comprises, consists essentially of, or consists of a homodimer or a heterodimer of one or more Type II nucleases. In some embodiments of the fusion protein, the effector molecule comprises, consists essentially of, or consists of a homodimer or a heterodimer of a Type II nuclease. In some embodiments, the Type II nuclease comprises one or more of AciI, MnlI, AlwI, BbvI, BccI, BceAI, BsmAI, BsmFI, BspCNI, BsrI, BtsCI, HgaI, HphI, HpyAV, MbolI, MylI, PleI, SfaNI, AcuI, BciVI, BfuAI, BmgBI, BmrI, BpmI, BpuEI, BsaI, BseRI, BsgI, BsmI, BspMI, BsrBI, BsrBI, BsrDI, BtgZI, BtsI, EarI, EciI, MmeI, NmeAIII, BbvCI, Bpu10I, BspQI, SapI, BaeI, BsaXI, CspCI, BfiI, MboII, Acc36I or Clo051.

In some embodiments, effector molecules, including those effector molecules comprising a homodimer or a heterodimer, comprise, consist essentially of, or consist of, Clo051, BfiI or BmrI. In some embodiments, effector molecules, including those effector molecules comprising a homodimer or a heterodimer, comprise, consist essentially of, or consist of, a Cas9, a Cas9 nuclease domain or a fragment thereof that forms a heterodimer with Clo051, BfiI or BmrI. In some embodiments, effector molecules, including those effector molecules comprising a homodimer or a heterodimer, comprise, consist essentially of, or consist of, a catalytically-inactive form of Cas9 (e.g. dCas9 or dSaCas9) or a fragment thereof that forms a heterodimer with Clo051. An exemplary Clo05 1 nuclease domain may comprise consist essentially of or consist of the amino acid sequence of:

EGIKSNISLLKDELRGQISHISHEYLSLIDLAFDSKQNRLFEMKVLELLV
NEYGFKGRHLGGSRKPDGIVYSTTLEDNFGIIVDTKAYSEGYSLPISQAD
EMERYVRENSNRDEEVNPNKWWENFSEEVKKYYFVFISGSFKGKFEEQLR
RLSMTTGVNGSAVNVVNLLLGAEKIRSGEMTIEELERAMENNSEFILKY
(SEQ ID NO: 386, SEQ ID NO: 34 of WO2018/169948).

In some embodiments, effector molecules, including those effector molecules comprising a homodimer or a heterodimer, comprise, consist essentially of, or consist of, a DNA-binding domain of a TALEN, or TAL protein, derived from Xanthomonas or Ralstonia. In some embodiments, effector molecules, including those effector molecules comprising a homodimer or a heterodimer, comprise, consist essentially of, or consist of, a DNA-binding domain of a TALEN, or TAL protein, derived from Xanthomonas or Ralstonia that forms a homodimer or a heterodimer with Clo051, BfiI or BmrI. In some embodiments, effector molecules, including those effector molecules comprising a homodimer or a heterodimer, comprise, consist essentially of, or consist of, a DNA-binding domain of a TALEN, or TAL protein, derived from Xanthomonas or Ralstonia that forms a homodimer or a heterodimer with Clo051.

### Linkages

In some embodiments, the fusion protein comprises, consists essentially of, or consists of, a DNA localization component and an effector molecule. In some embodiments, the nucleic acid sequences encoding one or more components of the fusion protein can be operably linked, for example, in an expression vector. In some embodiments, the fusion proteins are chimeric proteins. In some embodiments, the fusion proteins are encoded by one or more recombinant nucleic acid sequences. In some embodiments, the fusion proteins also include a linker region to operatively-link two components of the fusion protein. For example, in embodiments, the fusion protein comprises, consists essentially of, or consists, of a DNA localization component and an effector molecule, operatively-linked by a linker region. In some embodiments, the DNA localization component, the linker region, and the effector molecule can be encoded by one or more nucleic acid sequences inserted into an expression cassette and/or expression vector such that translation of the nucleic acid sequence results in the fusion protein. in embodiments, the fusion protein can comprise a non-covalent linkage between the DNA localization component and the effector molecule. The non-covalent linkage can comprise an antibody, an antibody fragment, an antibody mimetic, or a scaffold protein.

### Fusion Proteins

In some embodiments, the DNA localization component comprises, consists essentially of or consists of, at least one gRNA, and the effector molecule comprises, consists essentially of, or consists of a Cas9, a Cas9 nuclease domain, or a fragment thereof. In some embodiments, the DNA localization component comprises, consists essentially of, or consists of, at least one gRNA, and the effector molecule comprises, consists essentially of, or consist of an inactivated Cas9 (dCas9) or an inactivated nuclease domain. In some embodiments, the DNA localization component comprises, consists essentially of, or consists of, at least one gRNA, and the effector molecule comprises, consists essentially of, or consist of an inactivated small Cas9 (dSaCas9). In some embodiments, the effector molecule comprises, consists essentially of, or consists of a Cas9, dCas9, dSaCas9, or nuclease domain thereof, and a second endonuclease. The second endonuclease can comprise, consist essentially of, or consist of a Type IIS endonuclease, including, but not limited to, one or more of AciI, MnlI, AlwI, BbvI, BccI, BceAI, BsmAI, BsmFI, BspCNI, BsrI, BtsCI, HgaI, HphI, HpyAV, MbolI, MylI, PleI, SfaNI, AcuI, BciVI, BfuAI, BmgBI, BmrI, BpmI, BpuEI, BsaI, BseRI, BsgI, BsmI, BspMI, BsrBI, BsrBI, BsrDI, BtgZI, BtsI, EarI, EciI, MmeI, NmeAIII, BbvCI, Bpu10I, BspQI, SapI, BaeI, BsaXI, CspCI, BfiI, MboII, Acc36I, FokI or Clo051.

In some embodiments of the fusion proteins, the DNA localization component comprises, consists essentially of, or consists of, a DNA-binding domain of a transcription activator-like effector nuclease (TALEN, also referred to as a TAL protein), and the effector molecule comprises, consists essentially of, or consists of, an endonuclease. In some embodiments of the fusion proteins of the disclosure, the DNA localization component comprises, consists essentially of, or consists of, a DNA-binding domain of a TALEN, or TAL protein, derived from Xanthomonas or Ralstonia, and the effector molecule comprises, consists essentially of, or consists of, a Type IIS endonuclease, including, but not limited to, one or more of AciI, MnlI, AlwI, BbvI, BccI, BceAI, BsmAI, BsmFI, BspCNI, BsrI, BtsCI, HgaI, HphI, HpyAV, MbolI, MylI, PleI, SfaNI, AcuI, BciVI, BfuAI, BmgBI, BmrI, BpmI, BpuEI, BsaI, BseRI, BsgI, BsmI, BspMI, BsrBI, BsrBI, BsrDI, BtgZI, BtsI, EarI, EciI, MmeI, NmeAIII, BbvCI, Bpu10I, BspQI, SapI, BaeI, BsaXI, CspCI, BfiI, MboII, Acc36I or Clo051.

In certain embodiments, an exemplary dCas9-Clo051 fusion protein may comprise, consist essentially of or consist of the amino acid sequence of SEQ ID NO: 305 or 307 of WO2019/126578 or the nucleic acid sequence of SEQ ID NO: 306 or 308 of WO2019/126578.

### Constructs

In some embodiments, the nuclease domain comprises, consists essentially of, or consists of, a dCas9 and Clo051. In some embodiments, the nuclease domain comprises, consists essentially of, or consists of, a dSaCas9 and Clo051. In some embodiments, the nuclease domain comprises, consists essentially of, or consists of, a Xanthomonas-TALE and Clo051. In some embodiments, the nuclease domain comprises, consists essentially of, or consists of, a Ralstonia-TALE and Clo051. In some embodiments, the fusion protein comprises dCas9-Clo051, dSaCas9-Clo051, Xanthomonas-TALE-Clo051, or Ralstonia-TALE-Clo051. In some embodiments, a vector encoding the fusion protein comprises Csy4-T2A-Clo051-G4Slinker-dCas9 (Streptoccocus pyogenes) or pRT1-Clo051-dCas9 double NLS.

According to some embodiments, a Cas-CLOVER system comprises a fusion protein comprising a DNA localization component and an effector molecule, wherein the DNA localization component hybridizes to a target sequence of a DNA molecule in a TIL, wherein the DNA molecule encodes and the TIL expresses at least one immune checkpoint molecule, and the effector molecule cleaves the DNA molecule, whereby expression of the at least one immune checkpoint molecule is altered.

According to particular embodiments, a Cas-CLOVER method comprises silencing or reducing the expression of one or more immune checkpoint genes in TILs by introducing a Cas-CLOVER system (e.g., dCas9-Clo051, dSaCas9-Clo051, Xanthomonas-TALE-Clo051, or Ralstonia-TALE-Clo051 fusion protein) specific to a target DNA sequence of the immune checkpoint gene(s). The fusion protein may be delivered as DNA, mRNA, protein. Upon contact of the genome with the Cas-CLOVER system, one or more strand of the target double-stranded DNA may be cut. If the cut is made in the presence of one or more DNA repair pathways or components thereof, the Cas-CLOVER method either interrupts gene expression or modifies the genomic sequence by insertion, deletion, or substitution of one or more base pairs. DSBs may be repaired in the cells by non-homologous end joining (NHEJ), a mechanism which frequently causes insertions or deletions (indels) in the DNA. Indels often lead to frameshifts, creating loss of function alleles; for example, by causing premature stop codons within the open reading frame (ORF) of the targeted gene. According to certain embodiments, the result is a loss-of-function mutation within the targeted immune checkpoint gene.

Alternatively, DSBs induced by Cas-CLOVER systems may be repaired by homology-directed repair (HDR) instead of NHEJ. While NHEJ-mediated DSB repair often disrupts the open reading frame of the gene, homology directed repair (HDR) can be used to generate specific nucleotide changes ranging from a single nucleotide change to large insertions. According to some embodiments, HDR is used for gene editing immune checkpoint genes by delivering a DNA repair template containing the desired sequence into the TILs with the Cas-CLOVER system. The repair template preferably contains the desired edit as well as additional homologous sequence immediately upstream and downstream of the target gene (often referred to as left and right homology arms).

Non-limiting examples of genes that may be silenced or inhibited by permanently gene-editing TILs via a Cas-CLOVER method include PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.).

Examples of systems, methods, and compositions for altering the expression of a target gene sequence by a Cas-CLOVER method, and which may be used in accordance with embodiments of the present invention, are described in WO2019126578, US2017/0107541, US2017/0114149, US2018/0187185, and U.S. Pat. No. 10,415,024, the contents of which are incorporated herein by reference in their entirety. Resources for carrying out Cas-CLOVER methods, such as CLOVER mRNA and Cas-CLOVER mRNA constructs, are commercially available from companies such as Demeetra and Hera Biolabs.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system comprising a Cas-CLOVER system, which at least one gene editor system modulates expression of at least one checkpoint protein in the plurality of cells of the second population of TILs.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days to obtain the second population of TILs, wherein the transition from step (c) to step (d) occurs without opening the system;
(e) sterile electroporating the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the electroporation step comprises the delivery of at least one gene editor system comprising a Cas-CLOVER system, which at least one gene editor system inhibits expression of at least one checkpoint protein in the plurality of cells of the second population of TILs.

According to some embodiments, a method for expanding tumor infiltrating lymphocytes (TILs) into a therapeutic population of TILs comprises:
(a) obtaining a first population of TILs from a tumor resected from a patient by processing a tumor sample obtained from the patient into multiple tumor fragments;
(b) adding the tumor fragments into a closed system;
(c) performing a first expansion by culturing the first population of TILs in a cell culture medium comprising IL-2 and optionally comprising OKT-3 and/or a 4-1BB agonist antibody for about 3 to 11 days to produce a second population of TILs, wherein the first expansion is performed in a closed container providing a first gas-permeable surface area;
(d) stimulating the second population of TILs by adding OKT-3 and culturing for about 1 to 3 days, wherein the transition from step (c) to step (d) occurs without opening the system;
(e) temporarily disrupting the cell membranes of the second population of TILs to effect transfer of at least one gene editor into a plurality of cells in the second population of TILs;
(f) resting the second population of TILs for about 1 day;
(g) performing a second expansion by supplementing the cell culture medium of the second population of TILs with additional IL-2, optionally OKT-3 antibody, optionally an OX40 antibody, and antigen presenting cells (APCs), to produce a third population of TILs, wherein the second expansion is performed for about 7 to 11 days to obtain the third population of TILs, wherein the second expansion is performed in a closed container providing a second gas-permeable surface area, and wherein the transition from step (f) to step (g) occurs without opening the system;
(h) harvesting the therapeutic population of TILs obtained from step (g) to provide a harvested TIL population, wherein the transition from step (g) to step (h) occurs without opening the system, wherein the harvested population of TILs is a therapeutic population of TILs;
(i) transferring the harvested TIL population to an infusion bag, wherein the transfer from step (h) to (i) occurs without opening the system; and
(j) optionally cryopreserving the harvested TIL population using a cryopreservation medium,
   wherein the transfer of the at least one gene editor step comprises the delivery of a zinc finger nuclease system that effects expression of at least one immunomodulatory composition at the cell surface of and suppresses the expression of PD-1 and LAG-3 in the plurality of cells of the second population of TILs. In some embodiments, a microfluidic platform is used to temporarily disrupt the cell membranes of the third population of TILs. In some embodiments, the microfluidic platform is a SQZ vector-free microfluidic platform. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-7, IL-10, IL-12, IL-15, IL-18, IL-21, and a CD40 agonist (e.g., a CD40L or an agonistic CD40 binding domain). In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist.

In some embodiments, the invention provides the method described in any of the preceding paragraphs as applicable above modified such that the step of culturing the third or fourth population of TILs or the second expansion step is performed by culturing the third or fourth population of TILs or performing the second expansion for a first period of about 1-7 days, at the end of the first period the culture is split into a plurality of subcultures, each of the plurality of subcultures is cultured with additional IL-2 for a second period of about 3-7 days, and at the end of the second period the plurality of subcultures are combined to provide the expanded number of TILs or the therapeutic population of TILs.

In some embodiments, the invention provides the method described in any of the preceding paragraphs as applicable above modified such that the step of culturing the first population of TILs or the first expansion step is performed for about 3 days, 4 days, 5 days, 6 days, 7 days, 8 days, 9 days, 10 days, or 11 days.

In some embodiments, the invention provides the method described in any of the preceding paragraphs as applicable above modified such that the step of culturing the first population of TILs or the first expansion step is performed for about 4-11 days.

In some embodiments, the invention provides the method described in any of the preceding paragraphs as applicable above modified such that the step of culturing the first population of TILs or the first expansion step is performed for about 5-11 days.

In some embodiments, the invention provides the method described in any of the preceding paragraphs as applicable above modified such that the step of culturing the first population of TILs or the first expansion step is performed for about 6-11 days.

In some embodiments, the invention provides the method described in any of the preceding paragraphs as applicable above modified such that the step of culturing the first population of TILs or the first expansion step is performed for about 7-11 days.

In some embodiments, the invention provides the method described in any of the preceding paragraphs as applicable above modified such that the step of culturing the first population of TILs or the first expansion step is performed for about 8-11 days.

In some embodiments, the invention provides the method described in any of the preceding paragraphs as applicable above modified such that the step of culturing the first population of TILs or the first expansion step is performed for about 9-11 days.

In some embodiments, the invention provides the method described in any of the preceding paragraphs as applicable above modified such that the step of culturing the first population of TILs or the first expansion step is performed for about 10-11 days.

In some embodiments, the invention provides the method described in any of the preceding paragraphs as applicable above modified such that the step of culturing the first population of TILs or the first expansion step is performed for about 4-10 days.

In some embodiments, the invention provides the method described in any of the preceding paragraphs as applicable above modified such that the step of culturing the first population of TILs or the first expansion step is performed for about 5-10 days.

In some embodiments, the invention provides the method described in any of the preceding paragraphs as applicable above modified such that the step of culturing the first population of TILs or the first expansion step is performed for about 6-10 days.

In some embodiments, the invention provides the method described in any of the preceding paragraphs as applicable above modified such that the step of culturing the first population of TILs or the first expansion step is performed for about 7-10 days.

In some embodiments, the invention provides the method described in any of the preceding paragraphs as applicable above modified such that the step of culturing the first population of TILs or the first expansion step is performed for about 8-10 days.

In some embodiments, the invention provides the method described in any of the preceding paragraphs as applicable above modified such that the step of culturing the first population of TILs or the first expansion step is performed for about 9-10 days.

In some embodiments, the invention provides the method described in any of the preceding paragraphs as applicable above modified such that the step of culturing the first population of TILs or the first expansion step is performed for about 4-9 days.

In some embodiments, the invention provides the method described in any of the preceding paragraphs as applicable above modified such that the step of culturing the first population of TILs or the first expansion step is performed for about 5-9 days.

In some embodiments, the invention provides the method described in any of the preceding paragraphs as applicable above modified such that the step of culturing the first population of TILs or the first expansion step is performed for about 6-9 days.

In some embodiments, the invention provides the method described in any of the preceding paragraphs as applicable above modified such that the step of culturing the first population of TILs or the first expansion step is performed for about 7-9 days.

In some embodiments, the invention provides the method described in any of the preceding paragraphs as applicable above modified such that the step of culturing the first population of TILs or the first expansion step is performed for about 8-9 days.

In some embodiments, the invention provides the method described in any of the preceding paragraphs as applicable above modified such that the step of culturing the first population of TILs or the first expansion step is performed for about 3-8 days.

In some embodiments, the invention provides the method described in any of the preceding paragraphs as applicable above modified such that the step of culturing the first population of TILs or the first expansion step is performed for about 3-7 days.

In some embodiments, the invention provides the method described in any of the preceding paragraphs as applicable above modified such that the step of culturing the first population of TILs or the first expansion step is performed for about 3-6 days.

In some embodiments, the invention provides the method described in any of the preceding paragraphs as applicable above modified such that the step of culturing the first population of TILs or the first expansion step is performed for about 3-5 days.

In some embodiments, the invention provides the method described in any of the preceding paragraphs as applicable above modified such that the step of culturing the first population of TILs or the first expansion step is performed for about 3-4 days.

In some embodiments, the invention provides the method described in any of the preceding paragraphs as applicable above modified such that the step of culturing the first population of TILs or the first expansion step is performed for about 4-8 days.

In some embodiments, the invention provides the method described in any of the preceding paragraphs as applicable above modified such that the step of culturing the first population of TILs or the first expansion step is performed for about 4-7 days.

In some embodiments, the invention provides the method described in any of the preceding paragraphs as applicable above modified such that the step of culturing the first population of TILs or the first expansion step is performed for about 4-6 days.

In some embodiments, the invention provides the method described in any of the preceding paragraphs as applicable above modified such that the step of culturing the first population of TILs or the first expansion step is performed for about 4-6 days.

In some embodiments, the invention provides the method described in any of the preceding paragraphs as applicable above modified such that the step of culturing the first population of TILs or the first expansion step is performed for about 5-8 days.

In some embodiments, the invention provides the method described in any of the preceding paragraphs as applicable above modified such that the step of culturing the first population of TILs or the first expansion step is performed for about 5-7 days.

In some embodiments, the invention provides the method described in any of the preceding paragraphs as applicable above modified such that the step of culturing the first population of TILs or the first expansion step is performed for about 5-6 days.

In some embodiments, the invention provides the method described in any of the preceding paragraphs as applicable above modified such that the step of culturing the first population of TILs or the first expansion step is performed for about 6-8 days.

In some embodiments, the invention provides the method described in any of the preceding paragraphs as applicable above modified such that the step of culturing the first population of TILs or the first expansion step is performed for about 6-7 days.

In some embodiments, the invention provides the method described in any of the preceding paragraphs as applicable above modified such that the step of culturing the first population of TILs or the first expansion step is performed for about 7-8 days.

In some embodiments, the invention provides the method described in any of the preceding paragraphs as applicable above modified such that the step of culturing the first population of TILs or the first expansion step is performed for about 4-5 days.

In some embodiments, the invention provides the method described in any of the preceding paragraphs as applicable above modified such that the step of culturing the first population of TILs or the first expansion step is performed for about 3 days.

In some embodiments, the invention provides the method described in any of the preceding paragraphs as applicable above modified such that the step of culturing the first population of TILs or the first expansion step is performed for about 4 days.

In some embodiments, the invention provides the method described in any of the preceding paragraphs as applicable above modified such that the step of culturing the first population of TILs or the first expansion step is performed for about 5 days.

In some embodiments, the invention provides the method described in any of the preceding paragraphs as applicable above modified such that the step of culturing the first population of TILs or the first expansion step is performed for about 6 days.

In some embodiments, the invention provides the method described in any of the preceding paragraphs as applicable above modified such that the step of culturing the first population of TILs or the first expansion step is performed for about 7 days.

In some embodiments, the invention provides the method described in any of the preceding paragraphs as applicable above modified such that the step of culturing the first population of TILs or the first expansion step is performed for about 8 days.

In some embodiments, the invention provides the method described in any of the preceding paragraphs as applicable above modified such that the step of culturing the first population of TILs or the first expansion step is performed for about 9 days.

In some embodiments, the invention provides the method described in any of the preceding paragraphs as applicable above modified such that the step of culturing the first population of TILs or the first expansion step is performed for about 10 days.

In some embodiments, the invention provides the method described in any of the preceding paragraphs as applicable above modified such that the step of culturing the first population of TILs or the first expansion step is performed for about 11 days.

In some embodiments, the invention provides the method described in any of the preceding paragraphs as applicable above modified such that the activating step is performed for about 1 day, 2 days, 3 days, 4 days, 5 days, 6 days or 7 days.

In some embodiments, the invention provides the method described in any of the preceding paragraphs as applicable above modified such that the activating step is performed for about 2-7 days.

In some embodiments, the invention provides the method described in any of the preceding paragraphs as applicable above modified such that the activating step is performed for about 3-7 days.

In some embodiments, the invention provides the method described in any of the preceding paragraphs as applicable above modified such that the activating step is performed for about 4-7 days.

In some embodiments, the invention provides the method described in any of the preceding paragraphs as applicable above modified such that the activating step is performed for about 5-7 days.

In some embodiments, the invention provides the method described in any of the preceding paragraphs as applicable above modified such that the activating step is performed for about 6-7 days.

In some embodiments, the invention provides the method described in any of the preceding paragraphs as applicable above modified such that the activating step is performed for about 1-6 days.

In some embodiments, the invention provides the method described in any of the preceding paragraphs as applicable above modified such that the activating step is performed for about 1-5 days.

In some embodiments, the invention provides the method described in any of the preceding paragraphs as applicable above modified such that the activating step is performed for about 1-4 days.

In some embodiments, the invention provides the method described in any of the preceding paragraphs as applicable above modified such that the activating step is performed for about 1-3 days.

In some embodiments, the invention provides the method described in any of the preceding paragraphs as applicable above modified such that the activating step is performed for about 1-2 days.

In some embodiments, the invention provides the method described in any of the preceding paragraphs as applicable above modified such that the activating step is performed for about 2-6 days.

In some embodiments, the invention provides the method described in any of the preceding paragraphs as applicable above modified such that the activating step is performed for about 3-6 days.

In some embodiments, the invention provides the method described in any of the preceding paragraphs as applicable above modified such that the activating step is performed for about 4-6 days.

In some embodiments, the invention provides the method described in any of the preceding paragraphs as applicable above modified such that the activating step is performed for about 5-6 days.

In some embodiments, the invention provides the method described in any of the preceding paragraphs as applicable above modified such that the activating step is performed for about 3-5 days.

In some embodiments, the invention provides the method described in any of the preceding paragraphs as applicable above modified such that the activating step is performed for about 3-4 days.

In some embodiments, the invention provides the method described in any of the preceding paragraphs as applicable above modified such that the activating step is performed for about 2-5 days.

In some embodiments, the invention provides the method described in any of the preceding paragraphs as applicable above modified such that the activating step is performed for about 2-4 days.

In some embodiments, the invention provides the method described in any of the preceding paragraphs as applicable above modified such that the activating step is performed for about 2-3 days.

In some embodiments, the invention provides the method described in any of the preceding paragraphs as applicable above modified such that the activating step is performed for about 4-5 days.

In some embodiments, the invention provides the method described in any of the preceding paragraphs as applicable above modified such that the activating step is performed for about 1 day.

In some embodiments, the invention provides the method described in any of the preceding paragraphs as applicable above modified such that the activating step is performed for about 2 days.

In some embodiments, the invention provides the method described in any of the preceding paragraphs as applicable above modified such that the activating step is performed for about 3 days.

In some embodiments, the invention provides the method described in any of the preceding paragraphs as applicable above modified such that the activating step is performed for about 4 days.

In some embodiments, the invention provides the method described in any of the preceding paragraphs as applicable above modified such that the activating step is performed for about 5 days.

In some embodiments, the invention provides the method described in any of the preceding paragraphs as applicable above modified such that the activating step is performed for about 6 days.

In some embodiments, the invention provides the method described in any of the preceding paragraphs as applicable above modified such that the activating step is performed for about 7 days.

In some embodiments, the invention provides the method described in any of the preceding paragraphs as applicable above modified such that the stimulating step is performed from about 1 day, 2 days or 3 days.

In some embodiments, the invention provides the method described in any of the preceding paragraphs as applicable above modified such that the stimulating step is performed from about 1-2 days.

In some embodiments, the invention provides the method described in any of the preceding paragraphs as applicable above modified such that the stimulating step is performed from about 2-3 days.

In some embodiments, the invention provides the method described in any of the preceding paragraphs as applicable above modified such that the stimulating step is performed from about 1 day.

In some embodiments, the invention provides the method described in any of the preceding paragraphs as applicable above modified such that the stimulating step is performed from about 2 days.

In some embodiments, the invention provides the method described in any of the preceding paragraphs as applicable above modified such that the stimulating step is performed from about 3 days.

In some embodiments, the invention provides the method described in any of the preceding paragraphs as applicable above modified such that the step of culturing the third or fourth population of TILs or the second expansion step is performed for about 5 days, 6 days, 7 days, 8 days, 9 days, 10 days, 11 days, 12 days, 13 days, 14 days or 15 days.

In some embodiments, the invention provides the method described in any of the preceding paragraphs as applicable above modified such that the step of culturing the third or fourth population of TILs or the second expansion step is performed for about 6-15 days.

In some embodiments, the invention provides the method described in any of the preceding paragraphs as applicable above modified such that the step of culturing the third or fourth population of TILs or the second expansion step is performed for about 7-15 days.

In some embodiments, the invention provides the method described in any of the preceding paragraphs as applicable above modified such that the step of culturing the third or fourth population of TILs or the second expansion step is performed for about 8-15 days.

In some embodiments, the invention provides the method described in any of the preceding paragraphs as applicable above modified such that the step of culturing the third or fourth population of TILs or the second expansion step is performed for about 9-15 days.

In some embodiments, the invention provides the method described in any of the preceding paragraphs as applicable above modified such that the step of culturing the third or fourth population of TILs or the second expansion step is performed for about 10-15 days.

In some embodiments, the invention provides the method described in any of the preceding paragraphs as applicable above modified such that the step of culturing the third or fourth population of TILs or the second expansion step is performed for about 11-15 days.

In some embodiments, the invention provides the method described in any of the preceding paragraphs as applicable above modified such that the step of culturing the third or fourth population of TILs or the second expansion step is performed for about 12-15 days.

In some embodiments, the invention provides the method described in any of the preceding paragraphs as applicable above modified such that the step of culturing the third or fourth population of TILs or the second expansion step is performed for about 13-15 days.

In some embodiments, the invention provides the method described in any of the preceding paragraphs as applicable above modified such that the step of culturing the third or fourth population of TILs or the second expansion step is performed for about 14-15 days.

In some embodiments, the invention provides the method described in any of the preceding paragraphs as applicable above modified such that the step of culturing the third or fourth population of TILs or the second expansion step is performed for about 5-14 days.

In some embodiments, the invention provides the method described in any of the preceding paragraphs as applicable above modified such that the step of culturing the third or fourth population of TILs or the second expansion step is performed for about 6-14 days.

In some embodiments, the invention provides the method described in any of the preceding paragraphs as applicable above modified such that the step of culturing the third or fourth population of TILs or the second expansion step is performed for about 7-14 days.

In some embodiments, the invention provides the method described in any of the preceding paragraphs as applicable above modified such that the step of culturing the third or fourth population of TILs or the second expansion step is performed for about 8-14 days.

In some embodiments, the invention provides the method described in any of the preceding paragraphs as applicable above modified such that the step of culturing the third or fourth population of TILs or the second expansion step is performed for about 9-14 days.

In some embodiments, the invention provides the method described in any of the preceding paragraphs as applicable above modified such that the step of culturing the third or fourth population of TILs or the second expansion step is performed for about 10-14 days.

In some embodiments, the invention provides the method described in any of the preceding paragraphs as applicable above modified such that the step of culturing the third or fourth population of TILs or the second expansion step is performed for about 11-14 days.

In some embodiments, the invention provides the method described in any of the preceding paragraphs as applicable above modified such that the step of culturing the third or fourth population of TILs or the second expansion step is performed for about 12-14 days.

In some embodiments, the invention provides the method described in any of the preceding paragraphs as applicable above modified such that the step of culturing the third or fourth population of TILs or the second expansion step is performed for about 13-14 days.

In some embodiments, the invention provides the method described in any of the preceding paragraphs as applicable above modified such that the step of culturing the third or fourth population of TILs or the second expansion step is performed for about 5-13 days.

In some embodiments, the invention provides the method described in any of the preceding paragraphs as applicable above modified such that the step of culturing the third or fourth population of TILs or the second expansion step is performed for about 5-12 days.

In some embodiments, the invention provides the method described in any of the preceding paragraphs as applicable above modified such that the step of culturing the third or fourth population of TILs or the second expansion step is performed for about 5-11 days.

In some embodiments, the invention provides the method described in any of the preceding paragraphs as applicable above modified such that the step of culturing the third or fourth population of TILs or the second expansion step is performed for about 5-10 days.

In some embodiments, the invention provides the method described in any of the preceding paragraphs as applicable above modified such that the step of culturing the third or fourth population of TILs or the second expansion step is performed for about 5-9 days.

In some embodiments, the invention provides the method described in any of the preceding paragraphs as applicable above modified such that the step of culturing the third or fourth population of TILs or the second expansion step is performed for about 5-8 days.

In some embodiments, the invention provides the method described in any of the preceding paragraphs as applicable above modified such that the step of culturing the third or fourth population of TILs or the second expansion step is performed for about 5-7 days.

In some embodiments, the invention provides the method described in any of the preceding paragraphs as applicable above modified such that the step of culturing the third or fourth population of TILs or the second expansion step is performed for about 5-6 days.

In some embodiments, the invention provides the method described in any of the preceding paragraphs as applicable above modified such that the step of culturing the third or fourth population of TILs or the second expansion step is performed for about 6-13 days.

In some embodiments, the invention provides the method described in any of the preceding paragraphs as applicable above modified such that the step of culturing the third or fourth population of TILs or the second expansion step is performed for about 6-12 days.

In some embodiments, the invention provides the method described in any of the preceding paragraphs as applicable above modified such that the step of culturing the third or fourth population of TILs or the second expansion step is performed for about 6-11 days.

In some embodiments, the invention provides the method described in any of the preceding paragraphs as applicable above modified such that the step of culturing the third or fourth population of TILs or the second expansion step is performed for about 6-10 days.

In some embodiments, the invention provides the method described in any of the preceding paragraphs as applicable above modified such that the step of culturing the third or fourth population of TILs or the second expansion step is performed for about 6-9 days.

In some embodiments, the invention provides the method described in any of the preceding paragraphs as applicable above modified such that the step of culturing the third or fourth population of TILs or the second expansion step is performed for about 6-8 days.

In some embodiments, the invention provides the method described in any of the preceding paragraphs as applicable above modified such that the step of culturing the third or fourth population of TILs or the second expansion step is performed for about 6-7 days.

In some embodiments, the invention provides the method described in any of the preceding paragraphs as applicable above modified such that the step of culturing the third or fourth population of TILs or the second expansion step is performed for about 7-13 days.

In some embodiments, the invention provides the method described in any of the preceding paragraphs as applicable above modified such that the step of culturing the third or fourth population of TILs or the second expansion step is performed for about 7-12 days.

In some embodiments, the invention provides the method described in any of the preceding paragraphs as applicable above modified such that the step of culturing the third or fourth population of TILs or the second expansion step is performed for about 7-11 days.

In some embodiments, the invention provides the method described in any of the preceding paragraphs as applicable above modified such that the step of culturing the third or fourth population of TILs or the second expansion step is performed for about 7-10 days.

In some embodiments, the invention provides the method described in any of the preceding paragraphs as applicable above modified such that the step of culturing the third or fourth population of TILs or the second expansion step is performed for about 7-9 days.

In some embodiments, the invention provides the method described in any of the preceding paragraphs as applicable above modified such that the step of culturing the third or fourth population of TILs or the second expansion step is performed for about 7-8 days.

In some embodiments, the invention provides the method described in any of the preceding paragraphs as applicable above modified such that the step of culturing the third or fourth population of TILs or the second expansion step is performed for about 8-13 days.

In some embodiments, the invention provides the method described in any of the preceding paragraphs as applicable above modified such that the step of culturing the third or fourth population of TILs or the second expansion step is performed for about 8-12 days.

In some embodiments, the invention provides the method described in any of the preceding paragraphs as applicable above modified such that the step of culturing the third or fourth population of TILs or the second expansion step is performed for about 8-11 days.

In some embodiments, the invention provides the method described in any of the preceding paragraphs as applicable above modified such that the step of culturing the third or fourth population of TILs or the second expansion step is performed for about 8-10 days.

In some embodiments, the invention provides the method described in any of the preceding paragraphs as applicable above modified such that the step of culturing the third or fourth population of TILs or the second expansion step is performed for about 8-9 days.

In some embodiments, the invention provides the method described in any of the preceding paragraphs as applicable above modified such that the step of culturing the third or fourth population of TILs or the second expansion step is performed for about 9-13 days.

In some embodiments, the invention provides the method described in any of the preceding paragraphs as applicable above modified such that the step of culturing the third or fourth population of TILs or the second expansion step is performed for about 9-12 days.

In some embodiments, the invention provides the method described in any of the preceding paragraphs as applicable above modified such that the step of culturing the third or fourth population of TILs or the second expansion step is performed for about 9-11 days.

In some embodiments, the invention provides the method described in any of the preceding paragraphs as applicable above modified such that the step of culturing the third or fourth population of TILs or the second expansion step is performed for about 9-10 days.

In some embodiments, the invention provides the method described in any of the preceding paragraphs as applicable above modified such that the step of culturing the third or fourth population of TILs or the second expansion step is performed for about 10-13 days.

In some embodiments, the invention provides the method described in any of the preceding paragraphs as applicable above modified such that the step of culturing the third or fourth population of TILs or the second expansion step is performed for about 10-12 days.

In some embodiments, the invention provides the method described in any of the preceding paragraphs as applicable above modified such that the step of culturing the third or fourth population of TILs or the second expansion step is performed for about 10-11 days.

In some embodiments, the invention provides the method described in any of the preceding paragraphs as applicable above modified such that the step of culturing the third or fourth population of TILs or the second expansion step is performed for about 11-13 days.

In some embodiments, the invention provides the method described in any of the preceding paragraphs as applicable above modified such that the step of culturing the third or fourth population of TILs or the second expansion step is performed for about 11-12 days.

In some embodiments, the invention provides the method described in any of the preceding paragraphs as applicable above modified such that the step of culturing the third or fourth population of TILs or the second expansion step is performed for about 12-13 days.

In some embodiments, the invention provides the method described in any of the preceding paragraphs as applicable above modified such that the step of culturing the third or fourth population of TILs or the second expansion step is performed for about 5 days.

In some embodiments, the invention provides the method described in any of the preceding paragraphs as applicable above modified such that the step of culturing the third or fourth population of TILs or the second expansion step is performed for about 6 days.

In some embodiments, the invention provides the method described in any of the preceding paragraphs as applicable above modified such that the step of culturing the third or fourth population of TILs or the second expansion step is performed for about 7 days.

In some embodiments, the invention provides the method described in any of the preceding paragraphs as applicable above modified such that the step of culturing the third or fourth population of TILs or the second expansion step is performed for about 8 days.

In some embodiments, the invention provides the method described in any of the preceding paragraphs as applicable above modified such that the step of culturing the third or fourth population of TILs or the second expansion step is performed for about 9 days.

In some embodiments, the invention provides the method described in any of the preceding paragraphs as applicable above modified such that the step of culturing the third or fourth population of TILs or the second expansion step is performed for about 10 days.

In some embodiments, the invention provides the method described in any of the preceding paragraphs as applicable above modified such that the step of culturing the third or fourth population of TILs or the second expansion step is performed for about 11 days.

In some embodiments, the invention provides the method described in any of the preceding paragraphs as applicable above modified such that the step of culturing the third or fourth population of TILs or the second expansion step is performed for about 12 days.

In some embodiments, the invention provides the method described in any of the preceding paragraphs as applicable above modified such that the step of culturing the third or fourth population of TILs or the second expansion step is performed for about 13 days.

In some embodiments, the invention provides the method described in any of the preceding paragraphs as applicable above modified such that the step of culturing the third or fourth population of TILs or the second expansion step is performed for about 14 days.

In some embodiments, the invention provides the method described in any of the preceding paragraphs as applicable above modified such that the step of culturing the third or fourth population of TILs or the second expansion step is performed for about 15 days.

5. Short Hairpin RNA (shRNA) Methods

In some embodiments, the subject TILs described herein are modified to reduce endogenous protein expression using a short hairpin RNA (shRNA). In some embodiments, a nucleic acid is introduced into subject TIL that encodes for an RNA (e.g., a shRNA) that suppresses expression of an endogenous gene.

An “shRNA” as used herein is a short hairpin RNA, which is a sequence of RNA that makes a tight hairpin turn that can also be used to silence gene expression via RNA interference. Expression of shRNA in cells is typically accomplished by delivery of plasmids or through viral or bacterial vectors. shRNA is an advantageous mediator of RNAi in that it has a relatively low rate of degradation and turnover.

shRNA design is typically divided into two formats, the simple stem-loop shRNA and the microRNA-adapted shRNA. Basic shRNAs are modeled on precursor microRNA (pre-miRNA), and are cloned into viral vectors where they are transcribed under the control of a promoter. Exemplary promoters include, but are not limited to RNA Polymerase II (Pol II) and Polymerase III (Pol III) promoters (e.g., U6 and H1 promoters). shRNAs are produced as single-strand molecules of about 40-70 nucleotides in length, and form stem loop structures consisting of a 19-29 base-pair region of double-strand RNA (the stem) bridged by a region of single-strand RNA (the loop) and a short 3′ overhang. Once transcribed, shRNAs exit the nucleus, are cleaved at the loop by the nuclease Dicer in the cytoplasm, and enter the RNA-induced silencing complex (RISC) to direct cleavage and subsequent degradation of a target complementary mRNA. A microRNA-adapted shRNA consists of a shRNA stem structure with microRNA-like mismatches surrounded by the loop and flanking sequence of an endogenous microRNA. microRNA-adapted shRNAs are transcribed from RNA Polymerase 11 (Pol 11) promoters, cleaved by the endogenous RNase III Drosha enzyme in the nucleus, and then exported to the cytoplasm where they are processed by Dicer and loaded into the RISC complex. Studies have suggested that the use of a microRNA scaffold, which is processed by both Drosha and Dicer, may promote more efficient processing and reduce toxicity for in vivo RNAi. In some embodiments, the shRNA for TIL modification is a simple stem-loop shRNA. In some embodiments, the shRNA for TIL modification is a microRNA-adapted shRNA.

In some embodiments, the shRNA for subject TIL modification disclosed herein comprises a sequence complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, at least 23 nucleotides, at least 24 nucleotides, at least 25 nucleotides, at least 26 nucleotides, at least 27 nucleotides, at least 28 nucleotides, at least 29 nucleotides, or at least 30 nucleotides of the mRNA a target protein. In some embodiments, the shRNA for subject TIL modification disclosed herein comprises a sequence complementary to 13 nucleotides, 14 nucleotides, 15 nucleotides, 16 nucleotides, 17 nucleotides, 18 nucleotides, 19 nucleotides, 20 nucleotides, 21 nucleotides, 22 nucleotides, 23 nucleotides, 24 nucleotides, 25 nucleotides, 26 nucleotides, 27 nucleotides, 28 nucleotides, 29 nucleotides, or 30 nucleotides of the mRNA a target protein.

In some embodiments, the shRNA used for TIL modification is 40-50 nucleotides in length, 45-55 nucleotides in length, 50-60 nucleotides in length, 55-65 nucleotides in length, or 60-70 nucleotides in length.

shRNAs can be delivered to cells (e.g., the modified TILs provided herein) using any suitable method. For plasmids, typical transfection methods such as use of lipid transfection reagents or electroporation can be employed. In some embodiments, lentiviral particles are used to introduce the shRNA into the cell for modification. In some embodiments, a selectable marker is included to allow for elimination of cells in the culture that were not successfully transfected or transduced with the shRNA, thereby allowing for a pure culture of modified cells that include the shRNA.

In some embodiments, the shRNA is for reducing the expression of an immune checkpoint (e.g., any of the immune checkpoint proteins provided herein) in a subject TIL described herein. Non-limiting examples of genes that may be silenced or inhibited by permanently gene-editing TILs via a shRNA method include PD-1, TGIT, TET2, TGFβR2, PRA, BAFF (BR3), CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, BCOR, and any combinations thereof (e.g., two more more of the above immune checkpoints, such as PD-1 and TIGIT, PD-1 and LAG3, PD-1 and TIM3, TIM3 and CTLA-4, etc.). In some embodiments, one or more shRNAs is introduced into a TIL for modifying the expression of one or more of the following immune checkpoint proteins: PD-1, CTLA-4, LAG-3, TIGIT, TIM-3, Cish, TGFβ, PKA, CBLB, TOX, and any combinations thereof (e.g., two more more of the above immune checkpoints, such as PD-1 and TIGIT, PD-1 and LAG3, PD-1 and TIM3, TIM3 and CTLA-4, etc.). Exemplary shRNA sequences useful for modifying subject TILs are described in the Examples.

### V. Embodiments of Methods of Expanding Therapeutic T-Cells Including Peripheral Blood (PBLs) and/or Bone Marrow (MILs)

### A. Methods of Expanding Peripheral Blood Lymphocytes (PBLs) from Peripheral Blood

PBL Method 1. In some embodiments of the invention, PBLs are expanded using the processes described herein. In some embodiments of the invention, the method comprises obtaining a PBMC sample from whole blood. In some embodiments, the method comprises enriching T-cells by isolating pure T-cells from PBMCs using negative selection of a non-CD19+ fraction. On Day 0, the pure T-cells are cultured with anti-CD3/anti-CD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. PBLs are harvested on Day 14, beads are removed, and PBLs are counted and phenotyped. In some embodiments, the method comprises enriching T-cells by isolating pure T-cells from PBMCs using magnetic bead-based negative selection of a non-CD19+ fraction.

In some embodiments of the invention, PBL Method 1 is performed as follows: On Day 0, a cryopreserved PBMC sample is thawed and PBMCs are counted. T-cells are isolated using a Human Pan T-Cell Isolation Kit and LS columns (Miltenyi Biotec). The isolated T cells are counted and seeded at 5×10⁵ cells per well of a GRex 24-well plate and are co-cultured with DynaBeads® (anti-CD3/anti-CD28) at a 1:1 ratio with IL-2 at 3000 IU/ml in a total of 8 ml of CM2 media per well. On Day 4, the media in each well is exchanged from CM2 to AIM-V with fresh IL-2 at 3000 IU/ml. On Day 7, the expanded cells are harvested, counted, then cultured at 15×10⁶ cells per flask in GRex I0M flasks with IL-2 at 3000 IU/ml and DynaBeads® at a 1:1 ratio (beads:cells) in a total of 100 ml AIM-V media. On Day 11, the media is exchanged to CM-4 media supplemented with fresh IL-2 at 3000 IU/ml. On Day 14, the DynaBeads® are removed using a DynaMag Magnet (DynaMag™-15) and the cells are counted.

In some embodiments of the invention, PBL Method 1 is performed as follows: On Day 0, a cryopreserved PBMC sample is thawed and PBMCs are counted. T-cells are isolated using a Human Pan T-Cell Isolation Kit and LS columns (Miltenyi Biotec). The isolated T cells are counted and seeded at 5×10⁵ cells per well of a GRex 24-well plate and are co-cultured with DynaBeads® (anti-CD3/anti-CD28) at a 1:1 ratio with IL-2 at 3000 IU/ml in a total of 8 ml of CM2 media per well. On Day 4, the media in each well is exchanged from CM2 to AIM-V with fresh IL-2 at 3000 IU/ml. On Day 7, the PBLs are harvested, counted, then reseeded at 1×10⁶ cells per well of a new GRex-24 well plate with IL-2 at 3000 IU/ml and DynaBeads® at a 1:1 ratio (beads:cells) in a total of 8 ml AIM-V media. On Day 11, the media is exchanged to CM-4 media supplemented with fresh IL-2 at 3000 IU/ml. On Day 14, the DynaBeads® are removed using a DynaMag Magnet (DynaMag™-15) and the cells are counted.

PBL Method 2. In some embodiments of the invention, PBLs are expanded using PBL Method 2, which comprises obtaining a PBMC sample from whole blood. The T-cells from the PBMCs are enriched by incubating the PBMCs for at least three hours at 37° C. and then isolating the non-adherent cells. The non-adherent cells are the expanded similarly as PBL Method 1, that is, on Day 0, the non-adherent cells are cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. PBLs are harvested on Day 14, beads are removed, and PBLs are counted and phenotyped.

In some embodiments of the invention, PBL Method 2 is performed as follows: On Day 0, the cryopreserved PMBC sample is thawed and the PBMC cells are seeded at 6 million cells per well in a 6 well plate in CM-2 media and incubated for 3 hours at 37 degrees Celsius. After 3 hours, the non-adherent cells, which are the PBLs, are removed and counted. The PBLs are cultured with anti-CD3/anti-CD28 DynaBeads® in a 1:1 ratio of beads:cells, at 1×10⁶ cells per well and IL-2 at 3000 IU/ml in a total of 7 ml of CM-2 media in each well of a GRex 24-well plate. On Day 4, the media in each well is exchanged with AIM-V media and fresh IL-2 at 3000 IU/ml. On Day 7, the expanded cells are harvested, counted, then cultured at 15×10⁶ cells per flask in GRex I0M flasks with IL-2 at 3000 IU/ml and DynaBeads® at a 1:1 ratio (T-cells:beads) in a total of 100 ml AIM-V media. On Day 11, the media is changed to CM-4 media and supplemented with fresh IL-2 (3000 IU/ml). On Day 14, the DynaBeads are removed using a DynaMag™ Magnet (DynaMag™-15) and the cells are counted.

In some embodiments of the invention, PBL Method 2 is performed as follows: On Day 0, the cryopreserved PMBC sample is thawed and the PBMC cells are seeded at 6 million cells per well in a 6 well plate in CM-2 media and incubated for 3 hours at 37 degrees Celsius. After 3 hours, the non-adherent cells, which are the PBLs, are removed and counted. The PBLs are cultured with anti-CD3/anti-CD28 DynaBeads® in a 1:1 ratio of beads:cells, at 1×10⁶ cells per well and IL-2 at 3000 IU/ml in a total of 7 ml of CM-2 media in each well of a GRex 24-well plate. On Day 4, the media in each well is exchanged with AIM-V media and fresh IL-2 at 3000 IU/ml. On Day 7, the expanded cells are harvested, counted, then cultured at 1×10⁶ cells per well in a new GRex 24-well plate with IL-2 at 3000 IU/ml and DynaBeads® at a 1:1 ratio (T-cells:beads) in a total of 8 ml AIM-V media. On Day 11, the media is changed to CM-4 media and supplemented with fresh IL-2 (3000 IU/ml). On Day 14, the DynaBeads are removed using a DynaMag™ Magnet (DynaMag™-15) and the cells are counted.

PBL Method 3. In some embodiments of the invention, PBLs are expanded using PBL Method 3, which comprises obtaining a PBMC sample from peripheral blood. B-cells are isolated using a CD19+ selection and T-cells are selected using negative selection of the non-CD19+ fraction of the PBMC sample. On Day 0, the T-cells and B-cells are co-cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. PBLs are harvested on Day 14, beads are removed, and PBLs are counted and phenotyped.

In some embodiments of the invention, PBL Method 3 is performed as follows: On Day 0, cryopreserved PBMCs derived from peripheral blood are thawed and counted. CD19+ B-cells are sorted using a CD19 Multisort Kit, Human (Miltenyi Biotec). Of the non-CD19+ cell fraction, T-cells are purified using the Human Pan T-cell Isolation Kit and LS Columns (Miltenyi Biotec). The T-cells (PBLs) and B-cells are co-cultured at different ratios in a Grex 24-well plate in about 8 ml of CM2 media in the presence of IL-2 at about 3000 IU/ml. B-cell:T-cell ratios are 0.1:1; 1:1, and 10:1. The T-cell/B-cell co-culture is stimulated with antiCD3/antiCD28 antibodies (DynaBeads®) in a 1:1 ratio (beads:cells). On Day 4, the media is exchanged from CM2 to AIM-V media and additional IL-2 is added to the culture at 3000 IU/ml. On Day 7, the cells are harvested and counted and re-seeded on a new Grex 24-well plate in AIM-V media at a cell range of from about 1.5×10⁵ to about 4×10⁵ cells per well and stimulated with antiCD3/antiCD28 antibodies (DynaBeads®) in a 1:1 ratio (beads:cells), with additional IL-2 at 3000 IU/ml. On Day 14, the DynaBeads are removed using a DynaMag™ Magnet (DynaMag™-15) and the cells are counted.

In some embodiments, PBMCs are isolated from a whole blood sample. In some embodiments, the PBMC sample is used as the starting material to expand the PBLs. In some embodiments, the sample is cryopreserved prior to the expansion process. In other embodiments, a fresh sample is used as the starting material to expand the PBLs. In some embodiments of the invention, T-cells are isolated from PBMCs using methods known in the art. In some embodiments, the T-cells are isolated using a Human Pan T-cell isolation kit and LS columns. In some embodiments of the invention, T-cells are isolated from PBMCs using antibody selection methods known in the art, for example, CD19 negative selection.

In some embodiments of the invention, the process is performed over about 7 days, about 8 days, about 9 days, about 10 days, about 11 days, about 12 days, about 13 days, or about 14 days. In other embodiments, the process is performed over about 7 days. In other embodiments, the process is performed over about 14 days.

In some embodiments of the invention, the PBMCs are cultured with antiCD3/antiCD28 antibodies. In some embodiments, any available antiCD3/antiCD28 product is useful in the present invention. In some embodiments of the invention, the commercially available product used are DynaBeads®. In some embodiments, the DynaBeads® are cultured with the PBMCs in a ratio of 1:1 (beads:cells). In other embodiments, the antibodies are DynaBeads® cultured with the PBMCs in a ratio of 1.5:1, 2:1, 2.5:1, 3:1, 3.5:1, 4:1, 4.5:1, or 5:1 (beads:cells). In some embodiments of the invention, the antibody culturing steps and/or the step of restimulating cells with antibody is performed over a period of from about 2 to about 6 days, from about 3 to about 5 days, or for about 4 days. In some embodiments of the invention, the antibody culturing step is performed over a period of about 2 days, 3 days, 4 days, 5 days, or 6 days.

In some embodiments, the PBMC sample is cultured with IL-2. In some embodiments of the invention, the cell culture medium used for expansion of the PBLs from PBMCs comprises IL-2 at a concentration selected from the group consisting of about 100 IU/mL, about 200 IU/mL, about 300 IU/mL, about 400 IU/mL, about 100 IU/mL, about 100 IU/mL, about 100 IU/mL, about 100 IU/mL, about 100 IU/mL, about 500 IU/mL, about 600 IU/mL, about 700 IU/mL, about 800 IU/mL, about 900 IU/mL, about 1,000 IU/mL, about 1,100 IU/mL, about 1,200 IU/mL, about 1,300 IU/mL, about 1,400 IU/mL, about 1,500 IU/mL, about 1,600 IU/mL, about 1,700 IU/mL, about 1,800 IU/mL, about 1,900 IU/mL, about 2,000 IU/mL, about 2,100 IU/mL, about 2,200 IU/mL, about 2,300 IU/mL, about 2,400 IU/mL, about 2,500 IU/mL, about 2,600 IU/mL, about 2,700 IU/mL, about 2,800 IU/mL, about 2,900 IU/mL, about 3,000 IU/mL, about 3,100 IU/mL, about 3,200 IU/mL, about 3,300 IU/mL, about 3,400 IU/mL, about 3,500 IU/mL, about 3,600 IU/mL, about 3,700 IU/mL, about 3,800 IU/mL, about 3,900 IU/mL, about 4,000 IU/mL, about 4,100 IU/mL, about 4,200 IU/mL, about 4,300 IU/mL, about 4,400 IU/mL, about 4,500 IU/mL, about 4,600 IU/mL, about 4,700 IU/mL, about 4,800 IU/mL, about 4,900 IU/mL, about 5,000 IU/mL, about 5,100 IU/mL, about 5,200 IU/mL, about 5,300 IU/mL, about 5,400 IU/mL, about 5,500 IU/mL, about 5,600 IU/mL, about 5,700 IU/mL, about 5,800 IU/mL, about 5,900 IU/mL, about 6,000 IU/mL, about 6,500 IU/mL, about 7,000 IU/mL, about 7,500 IU/mL, about 8,000 IU/mL, about 8,500 IU/mL, about 9,000 IU/mL, about 9,500 IU/mL, and about 10,000 IU/mL.

In some embodiments of the invention, the starting cell number of PBMCs for the expansion process is from about 25,000 to about 1,000,000, from about 30,000 to about 900,000, from about 35,000 to about 850,000, from about 40, 000 to about 800,000, from about 45,000 to about 800,000, from about 50,000 to about 750,000, from about 55,000 to about 700,000, from about 60,000 to about 650,000, from about 65,000 to about 600,000, from about 70,000 to about 550,000, preferably from about 75,000 to about 500,000, from about 80,000 to about 450,000, from about 85,000 to about 400,000, from about 90,000 to about 350,000, from about 95,000 to about 300,000, from about 100,000 to about 250,000, from about 105,000 to about 200,000, or from about 110,000 to about 150,000. In some embodiments of the invention, the starting cell number of PBMCs is about 138,000, 140,000, 145,000, or more. In other embodiments, the starting cell number of PBMCs is about 28,000. In other embodiments, the starting cell number of PBMCs is about 62,000. In other embodiments, the starting cell number of PBMCs is about 338,000. In other embodiments, the starting cell number of PBMCs is about 336,000.

In some embodiments of the invention, the cells are grown in a GRex 24 well plate. In some embodiments of the invention, a comparable well plate is used. In some embodiments, the starting material for the expansion is about 5×10⁵ T-cells per well. In some embodiments of the invention, there are 1×10⁶ cells per well. In some embodiments of the invention, the number of cells per well is sufficient to seed the well and expand the T-cells.

In some embodiments of the invention, the fold expansion of PBLs is from about 20% to about 100%, 25% to about 95%, 30% to about 90%, 35% to about 85%, 40% to about 80%, 45% to about 75%, 50% to about 100%, or 25% to about 75%. In some embodiments of the invention, the fold expansion is about 25%. In other embodiments of the invention, the fold expansion is about 50%. In other embodiments, the fold expansion is about 75%.

In some embodiments of the invention, additional IL-2 may be added to the culture on one or more days throughout the process. In some embodiments of the invention, additional IL-2 is added on Day 4. In some embodiments of the invention, additional IL-2 is added on Day 7. In some embodiments of the invention, additional IL-2 is added on Day 11. In an other embodiment, additional IL-2 is added on Day 4, Day 7, and/or Day 11. In some embodiments of the invention, the cell culture medium may be changed on one or more days through the cell culture process. In some embodiments, the cell culture medium is changed on Day 4, Day 7, and/or Day 11 of the process. In some embodiments of the invention, the PBLs are cultured with additional IL-2 for a period of 1 day, 2 days, 3 days, 4 days, 5 days, 6 days, 7 days, 8 days, 9 days, 10 days, 11 days, 12 days, 13 days, or 14 days. In some embodiments of the invention, PBLs are cultured for a period of 3 days after each addition of IL-2.

In some embodiments, the cell culture medium is exchanged at least once time during the method. In some embodiments, the cell culture medium is exchanged at the same time that additional IL-2 is added. In other embodiments the cell culture medium is exchanged on at least one of Day 1, Day 2, Day 3, Day 4, Day 5, Day 6, Day 7, Day 8, Day 9, Day 10, Day 11, Day 12, Day 13, or Day 14. In some embodiments of the invention, the cell culture medium used throughout the method may be the same or different. In some embodiments of the invention, the cell culture medium is CM-2, CM-4, or AIM-V.

In some embodiments of the invention, T-cells may be restimulated with antiCD3/antiCD28 antibodies on one or more days throughout the 14-day expansion process. In some embodiments, the T-cells are restimulated on Day 7. In some embodiments, GRex 10M flasks are used for the restimulation step. In some embodiments of the invention, comparable flasks are used.

In some embodiments of the invention, the DynaBeads® are removed using a DynaMag™ Magnet, the cells are counted, and the cells are analyzed using phenotypic and functional analysis as further described in the Examples below. In some embodiments of the invention, antibodies are separated from the PBLs or MILs using methods known in the art. In any of the foregoing embodiments, magnetic bead-based selection of TILs, PBLs, or MILs is used.

In some embodiments of the invention, the PBMC sample is incubated for a period of time at a desired temperature effective to identify the non-adherent cells. In some embodiments of the invention, the incubation time is about 3 hours. In some embodiments of the invention, the temperature is about 370 Celsius. The non-adherent cells are then expanded using the process described above.

In some embodiments of the invention, the PBMCs are obtained from a patient who has been treated with ibrutinib or another ITK or kinase inhibitor, such ITK and kinase inhibitors as described elsewhere herein. In some embodiments of the invention, the ITK inhibitor is a covalent ITK inhibitor that covalently and irreversibly binds to ITK. In some embodiments of the invention, the ITK inhibitor is an allosteric ITK inhibitor that binds to ITK. In some embodiments of the invention, the PBMCs are obtained from a patient who has been treated with ibrutinib or another ITK inhibitor, including ITK inhibitors as described elsewhere herein, prior to obtaining a PBMC sample for use with any of the foregoing methods, including PBL Method 1, PBL Method 2, or PBL Method 3. In some embodiments of the invention, the ITK inhibitor treatment has been administered at least 1 time, at least 2, times, or at least 3 times or more. In some embodiments of the invention, PBLs that are expanded from patients pretreated with ibrutinib or another ITK inhibitor comprise less LAG3+, PD-1+ cells than those expanded from patients not pretreated with ibrutinib or another ITK inhibitor. In some embodiments of the invention PBLs that are expanded from patients pretreated with ibrutinib or another ITK inhibitor comprise increased levels of IFNγ production than those expanded from patients not pretreated with ibrutinib or another ITK inhibitor. In some embodiments of the invention, PBLs that are expanded from patients pretreated with ibrutinib or another ITK inhibitor comprise increased lytic activity at lower Effector:Target cell ratios than those expanded from patients not pretreated with ibrutinib or other ITK inhibitor. In some embodiments of the invention, patients pretreated with ibrutinib or other ITK inhibitor have higher fold-expansion as compared with untreated patients.

In some embodiments of the invention, the method includes a step of adding an ITK inhibitor to the cell culture. In some embodiments, the ITK inhibitor is added on one or more of Day 0, Day 1, Day 2, Day 3, Day 4, Day 5, Day 6, Day 7, Day 8, Day 9, Day 10, Day 11, Day 12, Day 13, or Day 14 of the process. In some embodiments, the ITK inhibitor is added on the days during the method when cell culture medium is exchanged. In some embodiments, the ITK inhibitor is added on Day 0 and when cell culture medium is exchanged. In some embodiments, the ITK inhibitor is added during the method when IL-2 is added. In some embodiments, the ITK inhibitor is added on Day 0, Day 4, Day 7, and optionally Day 11 of the method. In some embodiments of the invention, the ITK inhibitor is added at Day 0 and at Day 7 of the method. In some embodiments of the invention, the ITK inhibitor is one known in the art. In some embodiments of the invention, the ITK inhibitor is one described elsewhere herein.

In some embodiments of the invention, the ITK inhibitor is used in the method at a concentration of from about 0.1 nM to about SuM. In some embodiments, the ITK inhibitor is used in the method at a concentration of about 0.1 nM, 0.5 nM, 1 nM, 5 nM, 10 nM, 20 nM, 30 nM, 40 nM, 50 nM, 60 nM, 70 nM, 80 nM, 90 nM, 100 nM, 150 nM, 200 nM, 250 nM, 300 nM, 350 nM, 400 nM, 450 nM, 500 nM, 550 nM, 600 nM, 650 nM, 700 nM, 750 nM, 800 nM, 850 nM, 900 nM, 950 nM, 1 uM, 2 uM, 3 uM, 4 uM, or SuM.

In some embodiments of the invention, the method includes a step of adding an ITK inhibitor when the PBMCs are derived from a patient who has no prior exposure to an ITK inhibitor treatment, such as ibrutinib.

In some embodiments, the PBMC sample is from a subject or patient who has been optionally pre-treated with a regimen comprising a kinase inhibitor or an ITK inhibitor. In some embodiments, the tumor sample is from a subject or patient who has been pre-treated with a regimen comprising a kinase inhibitor or an ITK inhibitor. In some embodiments, the PBMC sample is from a subject or patient who has been pre-treated with a regimen comprising a kinase inhibitor or an ITK inhibitor, has undergone treatment for at least 1 month, at least 2 months, at least 3 months, at least 4 months, at least 5 months, at least 6 months, or 1 year or more. In other embodiments, the PBMCs are derived from a patient who is currently on an ITK inhibitor regimen, such as ibrutinib.

In some embodiments, the PBMC sample is from a subject or patient who has been pre-treated with a regimen comprising a kinase inhibitor or an ITK inhibitor and is refractory to treatment with a kinase inhibitor or an ITK inhibitor, such as ibrutinib.

In some embodiments, the PBMC sample is from a subject or patient who has been pre-treated with a regimen comprising a kinase inhibitor or an ITK inhibitor but is no longer undergoing treatment with a kinase inhibitor or an ITK inhibitor. In some embodiments, the PBMC sample is from a subject or patient who has been pre-treated with a regimen comprising a kinase inhibitor or an ITK inhibitor but is no longer undergoing treatment with a kinase inhibitor or an ITK inhibitor and has not undergone treatment for at least 1 month, at least 2 months, at least 3 months, at least 4 months, at least 5 months, at least 6 months, or at least 1 year or more. In other embodiments, the PBMCs are derived from a patient who has prior exposure to an ITK inhibitor, but has not been treated in at least 3 months, at least 6 months, at least 9 months, or at least 1 year.

In some embodiments of the invention, at Day 0, cells are selected for CD19+ and sorted accordingly. In some embodiments of the invention, the selection is made using antibody binding beads. In some embodiments of the invention, pure T-cells are isolated on Day 0 from the PBMCs. In some embodiments of the invention, at Day 0, the CD19+ B cells and pure T cells are co-cultured with antiCD3/antiCD28 antibodies for a minimum of 4 days. In some embodiments of the invention, on Day 4, IL-2 is added to the culture. In some embodiments of the invention, on Day 7, the culture is restimulated with antiCD3/antiCD28 antibodies and additional IL-2. In some embodiments of the invention, on Day 14, the PBLs are harvested.

In some embodiments of the invention, for patients that are not pre-treated with ibrutinib or other ITK inhibitor, 10-15 ml of Buffy Coat will yield about 5×10⁹ PBMC, which, in turn, will yield about 5.5×10⁷ starting cell material, and about 11×10⁹ PBLs at the end of the expansion process. In some embodiments of the invention, about 54×10⁶ PBMCs will yield about 6×10⁵ starting material, and about 1.2×10⁸ MIL (about a 205-fold expansion).

In some embodiments of the invention, for patients that are pre-treated with ibrutinib or other ITK inhibitor, the expansion process will yield about 20×10⁹ PBLs. In some embodiments of the invention, 40.3×10⁶ PBMCs will yield about 4.7×10⁵ starting cell material, and about 1.6×10⁸ PBLs (about a 338-fold expansion).

In some embodiments of the invention, the clinical dose of PBLs useful in the present invention for patients with chronic lymphocytic leukemia (CLL) is from about 0.1×10⁹ to about 15×10⁹ PBLs, from about 0.1×10⁹ to about 15×10⁹ PBLs, from about 0.12×10⁹ to about 12×10⁹ PBLs, from about 0.15×10⁹ to about 11×10⁹ PBLs, from about 0.2×10⁹ to about 10×10⁹ PBLs, from about 0.3×10⁹ to about 9×10⁹ PBLs, from about 0.4×10⁹ to about 8×10⁹ PBLs, from about 0.5×10⁹ to about 7×10⁹ PBLs, from about 0.6×10⁹ to about 6×10⁹ PBLs, from about 0.7×10⁹ to about 5×10⁹ PBLs, from about 0.8×10⁹ to about 4×10⁹ PBLs, from about 0.9×10⁹ to about 3×10⁹ PBLs, or from about 1×10⁹ to about 2×10⁹ PBLs.

In any of the foregoing embodiments, PBMCs may be derived from a whole blood sample, by apheresis, from the buffy coat, or from any other method known in the art for obtaining PBMCs.

### B. Methods of Expanding Marrow Infiltrating Lymphocytes (MILs) from PBMCs Derived from Bone Marrow

MIL Method 1. In some embodiments of the invention, a method for expanding MILs from PBMCs derived from bone marrow is described. In some embodiments of the invention, the method is performed over 14 days. In some embodiments, the method comprises obtaining bone marrow PBMCs and cryopreserving the PBMCs. On Day 0, the PBMCs are cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. MILs are harvested on Day 14, beads are removed, and MILs are optionally counted and phenotyped.

In some embodiments of the invention, MIL Method 1 is performed as follows: On Day 0, a cryopreserved PBMC sample derived from bone marrow is thawed and the PBMCs are counted. The PBMCs are co-cultured in a GRex 24-well plate at 5×10⁵ cells per well with anti-CD3/anti-CD28 antibodies (DynaBeads®) at a 1:1 ratio in about 8 ml per well of CM-2 cell culture medium (comprised of RPMI-1640, human AB serum, 1-glutamine, 2-mercaptoethanol, gentamicin sulfate, AIM-V media) in the presence of IL-2 at 3000 IU/ml. On Day 4, the cell culture media is exchanged with AIM-V supplemented with additional IL-2 at 3000 IU/ml. On Day 7, the expanded MILs are counted. 1×10⁶ cells per well are transferred to a new GRex 24-well plate and cultured with anti-CD3/anti-CD28 antibodies (DynaBeads®) at a 1:1 ratio in about 8 ml per well of AIM-V media in the presence of IL-2 at 3000 IU/ml. On Day 11, the cell culture media is exchanged from AIM-V to CM-4 (comprised of AIM-V media, 2 mM Glutamax, and 3000 IU/ml IL2). On Day 14, the DynaBeads® are removed using a DynaMag Magnet (DynaMag™15) and the MILs are counted.

MIL Method 2. In some embodiments of the invention, the method is performed over 7 days. In some embodiments, the method comprises obtaining PMBCs derived from bone marrow and cryopreserving the PBMCs. On Day 0, the PBMCs are cultured with antiCD3/antiCD28 antibodies (DynaBeads®) in a 3:1 ratio (beads:cells) and IL-2 at 3000 IU/ml. MILs are harvested on Day 7, beads are removed, and MILs are optionally counted and phenotyped.

In some embodiments of the invention, MIL Method 2 is performed as follows: On Day 0, a cryopreserved PBMC sample is thawed and the PBMCs are counted. The PBMCs are co-cultured in a GRex 24-well plate at 5×10⁵ cells per well with anti-CD3/anti-CD28 antibodies (DynaBeads®) at a 1:1 ratio in about 8 ml per well of CM-2 cell culture medium (comprised of RPMI-1640, human AB serum, 1-glutamine, 2-mercaptoethanol, gentamicin sulfate, AIM-V media) in the presence of IL-2 at 3000 IU/ml. On Day 7, the DynaBeads® are removed using a DynaMag Magnet (DynaMag™15) and the MILs are counted.

MIL Method 3. In some embodiments of the invention, the method comprises obtaining PBMCs from the bone marrow. On Day 0, the PBMCs are selected for CD3+/CD33+/CD20+/CD14+ and sorted, and the non-CD3+/CD33+/CD20+/CD14+ cell fraction is sonicated and a portion of the sonicated cell fraction is added back to the selected cell fraction. IL-2 is added to the cell culture at 3000 IU/ml. On Day 3, the PBMCs are cultured with antiCD3/antiCD28 antibodies (DynaBeads®) in a 1:1 ratio (beads:cells) and IL-2 at 3000 IU/ml. On Day 4, additional IL-2 is added to the culture at 3000 IU/ml. On Day 7, the culture is again stimulated with antiCD3/antiCD28 antibodies (DynaBeads®) in a 1:1 ratio (beads:cells), and additional IL-2 at 3000 IU/ml is added to the culture. On Day 11, IL-2 is added to the culture at 3000 IU/ml. MILs are harvested on Day 14, beads are removed, and MILs are optionally counted and phenotyped.

In some embodiments of the invention, MIL Method 3 is performed as follows: On Day 0, a cryopreserved sample of PBMCs is thawed and PBMCs are counted. The cells are stained with CD3, CD33, CD20, and CD14 antibodies and sorted using a S3e cell sorted (Bio-Rad). The cells are sorted into two fractions—an immune cell fraction (or the MIL fraction) (CD3+CD33+CD20+CD14+) and an AML blast cell fraction (non-CD3+CD33+CD20+CD14+). A number of cells from the AML blast cell fraction that is about equal to the number of cells from the immune cell fraction (or MIL fraction) to be seeded on a Grex 24-well plate is suspended in 100 ul of media and sonicated. In this example, about 2.8×10⁴ to about 3.38×10⁵ cells from the AML blast cell fraction is taken and suspended in 100 ul of CM2 media and then sonicated for 30 seconds. The 100 ul of sonicated AML blast cell fraction is added to the immune cell fraction in a Grex 24-well plate. The immune cells are present in an amount of about 2.8×10⁴ to about 3.38×10⁵ cells per well in about 8 ml per well of CM-2 cell culture medium in the presence of IL-2 at 6000 IU/ml and are cultured with the portion of AML blast cell fraction for about 3 days. On Day 3, anti-CD3/anti-CD28 antibodies (DynaBeads®) at a 1:1 ratio are added to the each well and cultured for about 1 day. On Day 4, the cell culture media is exchanged with AIM-V supplemented with additional IL-2 at 3000 IU/ml. On Day 7, the expanded MILs are counted. About 1.5×10⁵ to 4×10⁵ cells per well are transferred to a new GRex 24-well plate and cultured with anti-CD3/anti-CD28 antibodies (DynaBeads®) at a 1:1 ratio in about 8 ml per well of AIM-V medium in the presence of IL-2 at 3000 IU/ml. On Day 11, the cell culture media is exchanged from AIM-V to CM-4 (supplemented with IL-2 at 3000 IU/ml). On Day 14, the DynaBeads® are removed using a DynaMag Magnet (DynaMag™15) and the MILs are optionally counted.

In some embodiments of the invention, PBMCs are obtained from bone marrow. In some embodiments, the PBMCs are obtained from the bone marrow through apheresis, aspiration, needle biopsy, or other similar means known in the art. In some embodiments, the PBMCs are fresh. In other embodiments, the PBMCs are cryopreserved.

In some embodiments of the invention, the method is performed over about 7 days, about 8 days, about 9 days, about 10 days, about 11 days, about 12 days, about 13 days, or about 14 days. In other embodiments, the method is performed over about 7 days. In other embodiments, the method is performed over about 14 days.

In some embodiments of the invention, the PBMCs are cultured with antiCD3/antiCD28 antibodies. In some embodiments, any available antiCD3/antiCD28 product is useful in the present invention. In some embodiments of the invention, the commercially available product used are DynaBeads*. In some embodiments, the DynaBeads® are cultured with the PBMCs in a ratio of 1:1 (beads:cells). In other embodiments, the antibodies are DynaBeads® cultured with the PBMCs in a ratio of 1.5:1, 2:1, 2.5:1, 3:1, 3.5:1, 4:1, 4.5:1, or 5:1 (beads:cells). In any of the foregoing embodiments, magnetic bead-based selection of an immune cell fraction (or MIL fraction) (CD3+CD33+CD20+CD14+) or an AML blast cell fraction (non-CD3+CD33+CD20+CD14+) is used. In some embodiments of the invention, the antibody culturing steps and/or the step of restimulating cells with antibody is performed over a period of from about 2 to about 6 days, from about 3 to about 5 days, or for about 4 days. In some embodiments of the invention, the antibody culturing step is performed over a period of about 2 days, 3 days, 4 days, 5 days, or 6 days.

In some embodiments of the invention, the ratio of the number of cells from the AML blast cell fraction to the number of cells from the immune cell fraction (or MIL fraction) is about 0.1:1 to about 10:1. In other embodiments, the ratio is about 0.1:1 to about 5:1, about 0.1:1 to about 2:1, or about 1:1. In some embodiments of the invention, the AML blast cell fraction is optionally disrupted to break up cell aggregation. In some embodiments, the AML blast cell fraction is disrupted using sonication, homogenization, cell lysis, vortexing, or vibration. In other embodiments, the AML blast cell fraction is disrupted using sonication. In some embodiments of the invention, the non-CD3+, non-CD33+, non-CD20+, non-CD14+ cell fraction (AML blast fraction) is lysed using a suitable lysis method, including high temperature lysis, chemical lysis (such as organic alcohols), enzyme lysis, and other cell lysis methods known in the art.

In some embodiments of the invention, the cells from AML blast cell fraction are suspended at a concentration of from about 0.2×10⁵ to about 2×10⁵ cells per 100 uL and added to the cell culture with the immune cell fraction. In other embodiments, the concentration is from about 0.5×10⁵ to about 2×10⁵ cells per 100 uL, from about 0.7×10⁵ to about 2×10⁵ cells per 100 uL, from about 1×10⁵ to about 2×10⁵ cells per 100 uL, or from about 1.5×10⁵ to about 2×10⁵ cells per 100 uL.

In some embodiments, the PBMC sample is cultured with IL-2. In some embodiments of the invention, the cell culture medium used for expansion of the MILs comprises IL-2 at a concentration selected from the group consisting of about 100 IU/mL, about 200 IU/mL, about 300 IU/mL, about 400 IU/mL, about 100 IU/mL, about 100 IU/mL, about 100 IU/mL, about 100 IU/mL, about 100 IU/mL, about 500 IU/mL, about 600 IU/mL, about 700 IU/mL, about 800 IU/mL, about 900 IU/mL, about 1,000 IU/mL, about 1,100 IU/mL, about 1,200 IU/mL, about 1,300 IU/mL, about 1,400 IU/mL, about 1,500 IU/mL, about 1,600 IU/mL, about 1,700 IU/mL, about 1,800 IU/mL, about 1,900 IU/mL, about 2,000 IU/mL, about 2,100 IU/mL, about 2,200 IU/mL, about 2,300 IU/mL, about 2,400 IU/mL, about 2,500 IU/mL, about 2,600 IU/mL, about 2,700 IU/mL, about 2,800 IU/mL, about 2,900 IU/mL, about 3,000 IU/mL, about 3,100 IU/mL, about 3,200 IU/mL, about 3,300 IU/mL, about 3,400 IU/mL, about 3,500 IU/mL, about 3,600 IU/mL, about 3,700 IU/mL, about 3,800 IU/mL, about 3,900 IU/mL, about 4,000 IU/mL, about 4,100 IU/mL, about 4,200 IU/mL, about 4,300 IU/mL, about 4,400 IU/mL, about 4,500 IU/mL, about 4,600 IU/mL, about 4,700 IU/mL, about 4,800 IU/mL, about 4,900 IU/mL, about 5,000 IU/mL, about 5,100 IU/mL, about 5,200 IU/mL, about 5,300 IU/mL, about 5,400 IU/mL, about 5,500 IU/mL, about 5,600 IU/mL, about 5,700 IU/mL, about 5,800 IU/mL, about 5,900 IU/mL, about 6,000 IU/mL, about 6,500 IU/mL, about 7,000 IU/mL, about 7,500 IU/mL, about 8,000 IU/mL, about 8,500 IU/mL, about 9,000 IU/mL, about 9,500 IU/mL, and about 10,000 IU/mL.

In some embodiments of the invention, additional IL-2 may be added to the culture on one or more days throughout the method. In some embodiments of the invention, additional IL-2 is added on Day 4. In some embodiments of the invention, additional IL-2 is added on Day 7. In some embodiments of the invention, additional IL-2 is added on Day 11. In other embodiments, additional IL-2 is added on Day 4, Day 7, and/or Day 11. In some embodiments of the invention, the MILs are cultured with additional IL-2 for a period of 1 day, 2 days, 3 days, 4 days, 5 days, 6 days, 7 days, 8 days, 9 days, 10 days, 11 days, 12 days, 13 days, or 14 days. In some embodiments of the invention, MILs are cultured for a period of 3 days after each addition of IL-2.

In some embodiments, the cell culture medium is exchanged at least once time during the method. In some embodiments, the cell culture medium is exchanged at the same time that additional IL-2 is added. In other embodiments the cell culture medium is exchanged on at least one of Day 1, Day 2, Day 3, Day 4, Day 5, Day 6, Day 7, Day 8, Day 9, Day 10, Day 11, Day 12, Day 13, or Day 14. In some embodiments of the invention, the cell culture medium used throughout the method may be the same or different. In some embodiments of the invention, the cell culture medium is CM-2, CM-4, or AIM-V. In some embodiments of the invention, the cell culture medium exchange step on Day 11 is optional. In some embodiments of the invention, the starting cell number of PBMCs for the expansion process is from about 25,000 to about 1,000,000, from about 30,000 to about 900,000, from about 35,000 to about 850,000, from about 40,000 to about 800,000, from about 45,000 to about 800,000, from about 50,000 to about 750,000, from about 55,000 to about 700,000, from about 60,000 to about 650,000, from about 65,000 to about 600,000, from about 70,000 to about 550,000, preferably from about 75,000 to about 500,000, from about 80,000 to about 450,000, from about 85,000 to about 400,000, from about 90,000 to about 350,000, from about 95,000 to about 300,000, from about 100,000 to about 250,000, from about 105,000 to about 200,000, or from about 110,000 to about 150,000. In some embodiments of the invention, the starting cell number of PBMCs is about 138,000, 140,000, 145,000, or more. In other embodiments, the starting cell number of PBMCs is about 28,000. In other embodiments, the starting cell number of PBMCs is about 62,000. In other embodiments, the starting cell number of PBMCs is about 338,000. In other embodiments, the starting cell number of PBMCs is about 336,000.

In some embodiments of the invention, the fold expansion of MILs is from about 20% to about 100%, 25% to about 95%, 30% to about 90%, 35% to about 85%, 40% to about 80%, 45% to about 75%, 50% to about 100%, or 25% to about 75%. In some embodiments of the invention, the fold expansion is about 25%. In other embodiments of the invention, the fold expansion is about 50%. In other embodiments, the fold expansion is about 75%.

In some embodiments of the invention, MILs are expanded from 10-50 ml of bone marrow aspirate. In some embodiments of the invention, 10 ml of bone marrow aspirate is obtained from the patient. In other embodiments, 20 ml of bone marrow aspirate is obtained from the patient. In other embodiments, 30 ml of bone marrow aspirate is obtained from the patient. In other embodiments, 40 ml of bone marrow aspirate is obtained from the patient. In other embodiments, 50 ml of bone marrow aspirate is obtained from the patient.

In some embodiments of the invention, the number of PBMCs yielded from about 10-50 ml of bone marrow aspirate is about 5×10⁷ to about 10×10⁷ PBMCs. In other embodiments, the number of PMBCs yielded is about 7×10⁷ PBMCs.

In some embodiments of the invention, about 5×10⁷ to about 10×10⁷ PBMCs, yields about 0.5×10⁶ to about 1.5×10⁶ expansion starting cell material. In some embodiments of the invention, about 1×10⁶ expansion starting cell material is yielded.

In some embodiments of the invention, the total number of MILs harvested at the end of the expansion period is from about 0.01×10⁹ to about 1×10⁹, from about 0.05×10⁹ to about 0.9×10⁹, from about 0.1×10⁹ to about 0.85×10⁹, from about 0.15×10⁹ to about 0.7×10⁹, from about 0.2×10⁹ to about 0.65×10⁹, from about 0.25×10⁹ to about 0.6×10⁹, from about 0.3×10⁹ to about 0.55×10⁹, from about 0.35×10⁹ to about 0.5×10⁹, or from about 0.4×10⁹ to about 0.45×10⁹.

In some embodiments of the invention, 12×10⁶ PBMC derived from bone marrow aspirate yields approximately 1.4×10⁵ starting cell material, which yields about 1.1×10⁷ MILs at the end of the expansion process.

In some embodiments of the invention, the MILs expanded from bone marrow PBMCs using MIL Method 3 described above comprise a high proportion of CD8+ cells and lower number of LAG3+ and PD1+ cells as compared with MILs expanded using MIL Method 1 or MIL Method 2. In some embodiments of the invention, PBLs expanded from blood PBMC using MIL Method 3 described above comprise a high proportion of CD8+ cells and increased levels of IFNγ production as compared with PBLs expanded using MIL Method 1 or MIL Method 2.

In some embodiments of the invention, the clinical dose of MILs useful for patients with acute myeloid leukemia (AML) is in the range of from about 4×10⁸ to about 2.5×10⁹ MILs. In other embodiments, the number of MILs provided in the pharmaceutical compositions of the invention is 9.5×10⁸ MILs. In other embodiments, the number of MILs provided in the pharmaceutical compositions of the invention is 4.1×10⁸. In other embodiments, the number of MILs provided in the pharmaceutical compositions of the invention is 2.2×10⁹.

In any of the foregoing embodiments, PBMCs may be derived from a whole blood sample, from bone marrow, by apheresis, from the buffy coat, or from any other method known in the art for obtaining PBMCs.

### VI. Gen 2 TIL Manufacturing Processes

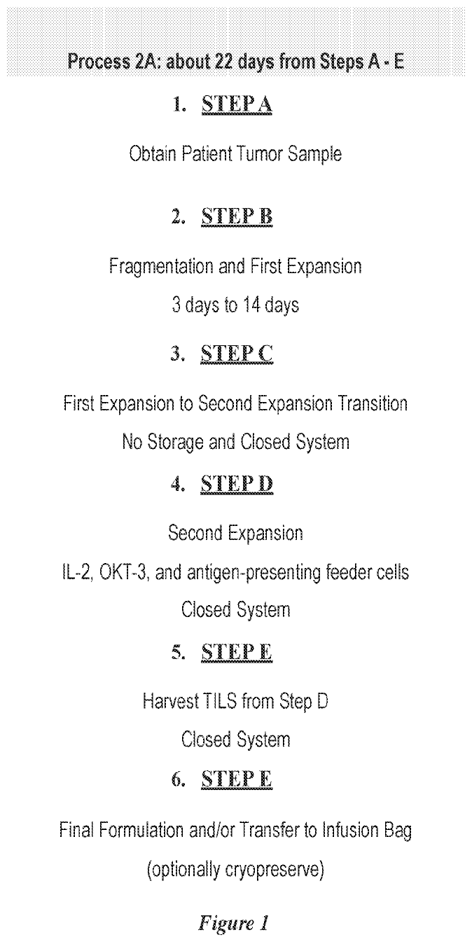

An exemplary family of TIL processes known as Gen 2 (also known as process 2A) containing some of these features is depicted in FIGS. 1 and 2. An embodiment of Gen 2 is shown in FIG. 2.

As discussed herein, the present invention can include a step relating to the restimulation of cryopreserved TILs to increase their metabolic activity and thus relative health prior to transplant into a patient, and methods of testing said metabolic health. As generally outlined herein, TILs are generally taken from a patient sample and manipulated to expand their number prior to transplant into a patient. In some embodiments, the TILs may be optionally genetically manipulated as discussed below.

In some embodiments, the TILs may be cryopreserved. Once thawed, they may also be restimulated to increase their metabolism prior to infusion into a patient.

In some embodiments, the first expansion (including processes referred to as the pre-REP as well as processes shown in FIG. 1 as Step A) is shortened to 3 to 14 days and the second expansion (including processes referred to as the REP as well as processes shown in FIG. 1 as Step B) is shorted to 7 to 14 days, as discussed in detail below as well as in the examples and figures. In some embodiments, the first expansion (for example, an expansion described as Step B in FIG. 1) is shortened to 11 days and the second expansion (for example, an expansion as described in Step D in FIG. 1) is shortened to 11 days. In some embodiments, the combination of the first expansion and second expansion (for example, expansions described as Step B and Step D in FIG. 1) is shortened to 22 days, as discussed in detail below and in the examples and figures.

The “Step” Designations A, B, C, etc., below are in reference to FIG. 1 and in reference to certain embodiments described herein. The ordering of the Steps below and in FIG. 1 is exemplary and any combination or order of steps, as well as additional steps, repetition of steps, and/or omission of steps is contemplated by the present application and the methods disclosed herein.

### A. STEP A: Obtain Patient Tumor Sample

In general, TILs are initially obtained from a patient tumor sample and then expanded into a larger population for further manipulation as described herein, optionally cryopreserved, restimulated as outlined herein and optionally evaluated for phenotype and metabolic parameters as an indication of TIL health.

A patient tumor sample may be obtained using methods known in the art, generally via surgical resection, needle biopsy, core biopsy, small biopsy, or other means for obtaining a sample that contains a mixture of tumor and TIL cells. In some embodiments, multilesional sampling is used. In some embodiments, surgical resection, needle biopsy, core biopsy, small biopsy, or other means for obtaining a sample that contains a mixture of tumor and TIL cells includes multilesional sampling (i.e., obtaining samples from one or more tumor sites and/or locations in the patient, as well as one or more tumors in the same location or in close proximity). In general, the tumor sample may be from any solid tumor, including primary tumors, invasive tumors or metastatic tumors. The tumor sample may also be a liquid tumor, such as a tumor obtained from a hematological malignancy. The solid tumor may be of lung tissue. In some embodiments, useful TILs are obtained from non-small cell lung carcinoma (NSCLC). The solid tumor may be of skin tissue. In some embodiments, useful TILs are obtained from a melanoma.

Once obtained, the tumor sample is generally fragmented using sharp dissection into small pieces of between 1 to about 8 mm³, with from about 2-3 mm³ being particularly useful. In some embodiments, the TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

Tumor dissociating enzyme mixtures can include one or more dissociating (digesting) enzymes such as, but not limited to, collagenase (including any blend or type of collagenase), Accutase™, Accumax™, hyaluronidase, neutral protease (dispase), chymotrypsin, chymopapain, trypsin, caseinase, elastase, papain, protease type XIV (pronase), deoxyribonuclease I (DNase), trypsin inhibitor, any other dissociating or proteolytic enzyme, and any combination thereof.

In some embodiments, the dissociating enzymes are reconstituted from lyophilized enzymes. In some embodiments, lyophilized enzymes are reconstituted in an amount of sterile buffer such as HBSS.

In some instances, collagenase (such as animal free-type 1 collagenase) is reconstituted in 10 mL of sterile HBSS or another buffer. The lyophilized stock enzyme may be at a concentration of 2892 PZ U/vial. In some embodiments, collagenase is reconstituted in 5 mL to 15 mL buffer. In some embodiments, after reconstitution the collagenase stock ranges from about 100 PZ U/mL-about 400 PZ U/mL, e.g., about 100 PZ U/mL-about 400 PZ U/mL, about 100 PZ U/mL-about 350 PZ U/mL, about 100 PZ U/mL-about 300 PZ U/mL, about 150 PZ U/mL-about 400 PZ U/mL, about 100 PZ U/mL, about 150 PZ U/mL, about 200 PZ U/mL, about 210 PZ U/mL, about 220 PZ U/mL, about 230 PZ U/mL, about 240 PZ U/mL, about 250 PZ U/mL, about 260 PZ U/mL, about 270 PZ U/mL, about 280 PZ U/mL, about 289.2 PZ U/mL, about 300 PZ U/mL, about 350 PZ U/mL, or about 400 PZ U/mL.

In some embodiments, neutral protease is reconstituted in 1 mL of sterile HBSS or another buffer. The lyophilized stock enzyme may be at a concentration of 175 DMC U/vial. In some embodiments, after reconstitution the neutral protease stock ranges from about 100 DMC/mL-about 400 DMC/mL, e.g., about 100 DMC/mL-about 400 DMC/mL, about 100 DMC/mL-about 350 DMC/mL, about 100 DMC/mL-about 300 DMC/mL, about 150 DMC/mL-about 400 DMC/mL, about 100 DMC/mL, about 110 DMC/mL, about 120 DMC/mL, about 130 DMC/mL, about 140 DMC/mL, about 150 DMC/mL, about 160 DMC/mL, about 170 DMC/mL, about 175 DMC/mL, about 180 DMC/mL, about 190 DMC/mL, about 200 DMC/mL, about 250 DMC/mL, about 300 DMC/mL, about 350 DMC/mL, or about 400 DMC/mL.

In some embodiments, DNAse I is reconstituted in 1 mL of sterile HBSS or another buffer. The lyophilized stock enzyme was at a concentration of 4 KU/vial. In some embodiments, after reconstitution the DNase I stock ranges from about 1 KU/mL-10 KU/mL, e.g., about 1 KU/mL, about 2 KU/mL, about 3 KU/mL, about 4 KU/mL, about 5 KU/mL, about 6 KU/mL, about 7 KU/mL, about 8 KU/mL, about 9 KU/mL, or about 10 KU/mL.

In some embodiments, the stock of enzymes is variable and the concentrations may need to be determined. In some embodiments, the concentration of the lyophilized stock can be verified. In some embodiments, the final amount of enzyme added to the digest cocktail is adjusted based on the determined stock concentration.

In some embodiments, the enzyme mixture includes about 10.2-ul of neutral protease (0.36 DMC U/mL), 21.3 μL of collagenase (1.2 PZ/mL) and 250-ul of DNAse I (200 U/mL) in about 4.7 mL of sterile HBSS.

As indicated above, in some embodiments, the TILs are derived from solid tumors. In some embodiments, the solid tumors are not fragmented. In some embodiments, the solid tumors are not fragmented and are subjected to enzymatic digestion as whole tumors. In some embodiments, the tumors are digested in in an enzyme mixture comprising collagenase, DNase, and hyaluronidase. In some embodiments, the tumors are digested in in an enzyme mixture comprising collagenase, DNase, and hyaluronidase for 1-2 hours. In some embodiments, the tumors are digested in in an enzyme mixture comprising collagenase, DNase, and hyaluronidase for 1-2 hours at 37° C., 5% CO₂. In some embodiments, the tumors are digested in in an enzyme mixture comprising collagenase, DNase, and hyaluronidase for 1-2 hours at 37° C., 5% CO₂ with rotation. In some embodiments, the tumors are digested overnight with constant rotation. In some embodiments, the tumors are digested overnight at 37° C., 5% CO₂ with constant rotation. In some embodiments, the whole tumor is combined with the enzymes to form a tumor digest reaction mixture.

In some embodiments, the tumors are digested in an enzyme mixture comprising collagenase, DNase, and neutral protease. In some embodiments, the tumors are digested in an enzyme mixture comprising collagenase, DNase, and neutral protease for 1-2 hours. In some embodiments, the tumors are digested in an enzyme mixture comprising collagenase, DNase, and neutral protease for 1-2 hours at 37° C., 5% CO₂. In some embodiments, the tumors are digested in an enzyme mixture comprising collagenase, DNase, and neutral protease for 1-2 hours at 37° C., 5% CO₂ with rotation. In some embodiments, the tumors are digested overnight with constant rotation. In some embodiments, the tumors are digested overnight at 37° C., 5% CO₂ with constant rotation. In some embodiments, the whole tumor is combined with the enzymes to form a tumor digest reaction mixture.

In some embodiments, the tumor is reconstituted with the lyophilized enzymes in a sterile buffer. In some embodiments, the buffer is sterile HBSS.

In some embodiments, the enzyme mixture comprises collagenase. In some embodiments, the collagenase is collagenase IV. In some embodiments, the working stock for the collagenase is a 100 mg/mL 10× working stock.

In some embodiments, the enzyme mixture comprises DNAse. In some embodiments, the working stock for the DNAse is a 10,000 IU/mL 10× working stock.

In some embodiments, the enzyme mixture comprises hyaluronidase. In some embodiments, the working stock for the hyaluronidase is a 10 mg/mL 10× working stock.

In some embodiments, the enzyme mixture comprises 10 mg/mL collagenase, 1000 IU/mL DNAse, and 1 mg/mL hyaluronidase.

In some embodiments, the enzyme mixture comprises 10 mg/mL collagenase, 500 IU/mL DNAse, and 1 mg/mL hyaluronidase.

In general, the harvested cell suspension is called a “primary cell population” or a “freshly harvested” cell population.

In some embodiments, fragmentation includes physical fragmentation, including for example, dissection as well as digestion. In some embodiments, the fragmentation is physical fragmentation. In some embodiments, the fragmentation is dissection. In some embodiments, the fragmentation is by digestion. In some embodiments, TILs can be initially cultured from enzymatic tumor digests and tumor fragments obtained from digesting or fragmenting a tumor sample obtained from a patient.

In some embodiments, where the tumor is a solid tumor, the tumor undergoes physical fragmentation after the tumor sample is obtained in, for example, Step A (as provided in FIG. 1). In some embodiments, the fragmentation occurs before cryopreservation. In some embodiments, the fragmentation occurs after cryopreservation. In some embodiments, the fragmentation occurs after obtaining the tumor and in the absence of any cryopreservation. In some embodiments, the tumor is fragmented and 10, 20, 30, 40 or more fragments or pieces are placed in each container for the first expansion. In some embodiments, the tumor is fragmented and 30 or 40 fragments or pieces are placed in each container for the first expansion. In some embodiments, the tumor is fragmented and 40 fragments or pieces are placed in each container for the first expansion. In some embodiments, the multiple fragments comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams. In some embodiments, the multiple fragments comprise about 4 fragments.

In some embodiments, the TILs are obtained from tumor fragments. In some embodiments, the tumor fragment is obtained by sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, the tumors are 1-4 mm×1-4 mm×1-4 mm. In some embodiments, the tumors are 1 mm×1 mm×1 mm. In some embodiments, the tumors are 2 mm×2 mm×2 mm. In some embodiments, the tumors are 3 mm×3 mm×3 mm. In some embodiments, the tumors are 4 mm×4 mm×4 mm.

In some embodiments, the tumors are resected in order to minimize the amount of hemorrhagic, necrotic, and/or fatty tissues on each piece. In some embodiments, the tumors are resected in order to minimize the amount of hemorrhagic tissue on each piece. In some embodiments, the tumors are resected in order to minimize the amount of necrotic tissue on each piece. In some embodiments, the tumors are resected in order to minimize the amount of fatty tissue on each piece.

In some embodiments, the tumor fragmentation is performed in order to maintain the tumor internal structure. In some embodiments, the tumor fragmentation is performed without performing a sawing motion with a scalpel. In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contains a large number of red blood cells or dead cells, a density gradient separation using Ficoll can be performed to remove these cells.

In some embodiments, the harvested cell suspension prior to the first expansion step is called a “primary cell population” or a “freshly harvested” cell population.

In some embodiments, cells can be optionally frozen after sample harvest and stored frozen prior to entry into the expansion described in Step B, which is described in further detail below, as well as exemplified in FIG. 1, as well as FIG. 8.

### 1. Pleural Effusion T-Cells and TILs

In some embodiments, the sample is a pleural fluid sample. In some embodiments, the source of the T-cells or TILs for expansion according to the processes described herein is a pleural fluid sample. In some embodiments, the sample is a pleural effusion derived sample. In some embodiments, the source of the T-cells or TILs for expansion according to the processes described herein is a pleural effusion derived sample. See, for example, methods described in U.S. Patent Publication US 2014/0295426, incorporated herein by reference in its entirety for all purposes.

In some embodiments, any pleural fluid or pleural effusion suspected of and/or containing TILs can be employed. Such a sample may be derived from a primary or metastatic lung cancer, such as NSCLC or SCLC. In some embodiments, the sample may be derived from secondary metastatic cancer cells which originated from another organ, e.g., breast, ovary, colon or prostate. In some embodiments, the sample for use in the expansion methods described herein is a pleural exudate. In some embodiments, the sample for use in the expansion methods described herein is a pleural transudate. Other biological samples may include other serous fluids containing TILs, including, e.g., ascites fluid from the abdomen or pancreatic cyst fluid. Ascites fluid and pleural fluids involve very similar chemical systems; both the abdomen and lung have mesothelial lines and fluid forms in the pleural space and abdominal spaces in the same matter in malignancies and such fluids in some embodiments contain TILs. In some embodiments, wherein the disclosed methods utilize pleural fluid, the same methods may be performed with similar results using ascites or other cyst fluids containing TILs.

In some embodiments, the pleural fluid is in unprocessed form, directly as removed from the patient. In some embodiments, the unprocessed pleural fluid is placed in a standard blood collection tube, such as an EDTA or Heparin tube, prior to further processing steps. In some embodiments, the unprocessed pleural fluid is placed in a standard CellSave® tube (Veridex) prior to further processing steps. In some embodiments, the sample is placed in the CellSave tube immediately after collection from the patient to avoid a decrease in the number of viable TILs. The number of viable TILs can decrease to a significant extent within 24 hours, if left in the untreated pleural fluid, even at 4° C. In some embodiments, the sample is placed in the appropriate collection tube within 1 hour, 5 hours, 10 hours, 15 hours, or up to 24 hours after removal from the patient. In some embodiments, the sample is placed in the appropriate collection tube within 1 hour, 5 hours, 10 hours, 15 hours, or up to 24 hours after removal from the patient at 4° C.

In some embodiments, the pleural fluid sample from the chosen subject may be diluted. In some embodiments, the dilution is 1:10 pleural fluid to diluent. In other embodiments, the dilution is 1:9 pleural fluid to diluent. In other embodiments, the dilution is 1:8 pleural fluid to diluent. In other embodiments, the dilution is 1:5 pleural fluid to diluent. In other embodiments, the dilution is 1:2 pleural fluid to diluent. In other embodiments, the dilution is 1:1 pleural fluid to diluent. In some embodiments, diluents include saline, phosphate buffered saline, another buffer or a physiologically acceptable diluent. In some embodiments, the sample is placed in the CellSave tube immediately after collection from the patient and dilution to avoid a decrease in the viable TILs, which may occur to a significant extent within 24-48 hours, if left in the untreated pleural fluid, even at 4° C. In some embodiments, the pleural fluid sample is placed in the appropriate collection tube within 1 hour, 5 hours, 10 hours, 15 hours, 24 hours, 36 hours, up to 48 hours after removal from the patient, and dilution. In some embodiments, the pleural fluid sample is placed in the appropriate collection tube within 1 hour, 5 hours, 10 hours, 15 hours, 24 hours, 36 hours, up to 48 hours after removal from the patient, and dilution at 4° C.

In still other embodiments, pleural fluid samples are concentrated by conventional means prior to further processing steps. In some embodiments, this pre-treatment of the pleural fluid is preferable in circumstances in which the pleural fluid must be cryopreserved for shipment to a laboratory performing the method or for later analysis (e.g., later than 24-48 hours post-collection). In some embodiments, the pleural fluid sample is prepared by centrifuging the pleural fluid sample after its withdrawal from the subject and resuspending the centrifugate or pellet in buffer. In some embodiments, the pleural fluid sample is subjected to multiple centrifugations and resuspensions, before it is cryopreserved for transport or later analysis and/or processing.

In some embodiments, pleural fluid samples are concentrated prior to further processing steps by using a filtration method. In some embodiments, the pleural fluid sample used in further processing is prepared by filtering the fluid through a filter containing a known and essentially uniform pore size that allows for passage of the pleural fluid through the membrane but retains the tumor cells. In some embodiments, the diameter of the pores in the membrane may be at least 4 μM. In other embodiments the pore diameter may be 5 μM or more, and in other embodiment, any of 6, 7, 8, 9, or 10 μM. After filtration, the cells, including TILs, retained by the membrane may be rinsed off the membrane into a suitable physiologically acceptable buffer. Cells, including TILs, concentrated in this way may then be used in the further processing steps of the method.

In some embodiments, pleural fluid sample (including, for example, the untreated pleural fluid), diluted pleural fluid, or the resuspended cell pellet, is contacted with a lytic reagent that differentially lyses non-nucleated red blood cells present in the sample. In some embodiments, this step is performed prior to further processing steps in circumstances in which the pleural fluid contains substantial numbers of RBCs. Suitable lysing reagents include a single lytic reagent or a lytic reagent and a quench reagent, or a lytic agent, a quench reagent and a fixation reagent. Suitable lytic systems are marketed commercially and include the BD Pharm Lyse™ system (Becton Dickenson). Other lytic systems include the Versalyse™ system, the FACSlyse™ system (Becton Dickenson), the Immunoprep™ system or Erythrolyse II system (Beckman Coulter, Inc.), or an ammonium chloride system. In some embodiments, the lytic reagent can vary with the primary requirements being efficient lysis of the red blood cells, and the conservation of the TILs and phenotypic properties of the TILs in the pleural fluid. In addition to employing a single reagent for lysis, the lytic systems useful in methods described herein can include a second reagent, e.g., one that quenches or retards the effect of the lytic reagent during the remaining steps of the method, e.g., Stabilyse™ reagent (Beckman Coulter, Inc.). A conventional fixation reagent may also be employed depending upon the choice of lytic reagents or the preferred implementation of the method.

In some embodiments, the pleural fluid sample, unprocessed, diluted or multiply centrifuged or processed as described herein above is cryopreserved at a temperature of about −140° C. prior to being further processed and/or expanded as provided herein.

### B. STEP B: First Expansion

In some embodiments, the present methods provide for obtaining young TILs, which are capable of increased replication cycles upon administration to a subject/patient and as such may provide additional therapeutic benefits over older TILs (i.e., TILs which have further undergone more rounds of replication prior to administration to a subject/patient). Features of young TILs have been described in the literature, for example in Donia, et al., Scand. J Immunol. 2012, 75, 157-167; Dudley, et al., Clin. Cancer Res. 2010, 16, 6122-6131; Huang, et al., J Immunother. 2005, 28, 258-267; Besser, et al., Clin. Cancer Res. 2013, 19, OF1-OF9; Besser, et al., J Immunother. 2009, 32:415-423; Robbins, et al., J Immunol. 2004, 173, 7125-7130; Shen, et al., J Immunother., 2007, 30, 123-129; Zhou, et al., J Immunother. 2005, 28, 53-62; and Tran, et al., J Immunother., 2008, 31, 742-751, each of which is incorporated herein by reference.

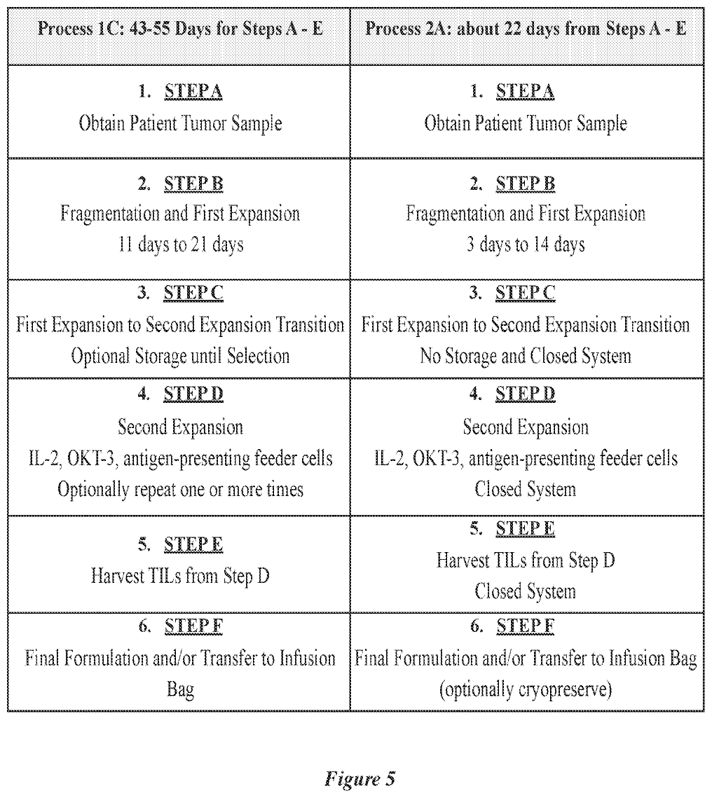

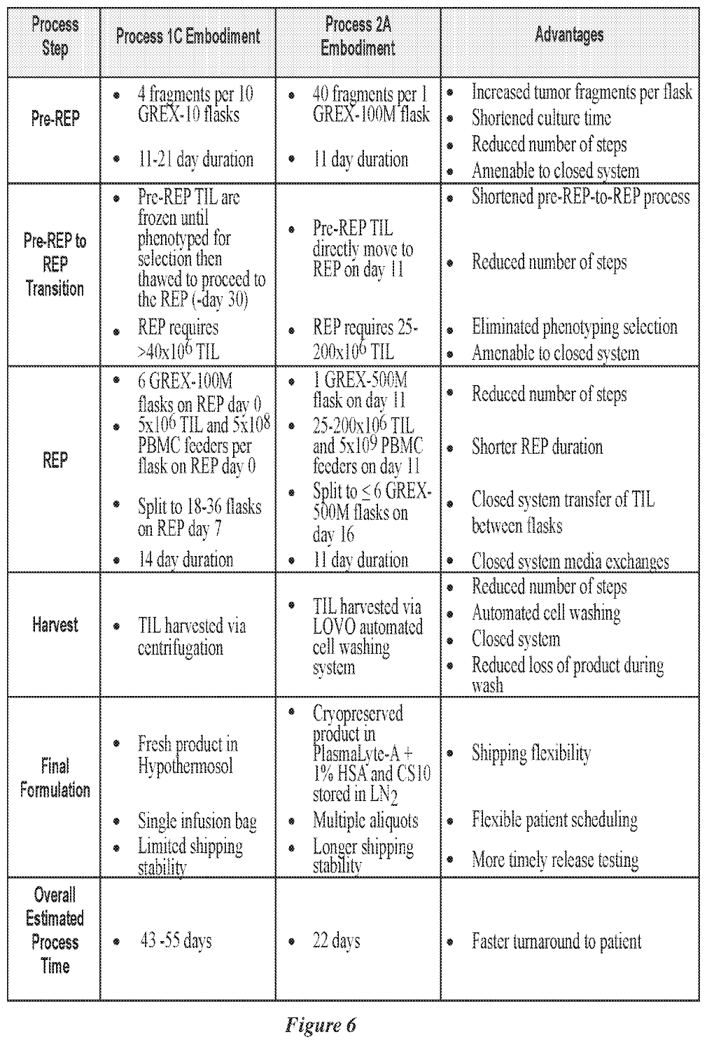

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained by the present method exhibit an increase in the T-cell repertoire diversity as compared to freshly harvested TILs and/or TILs prepared using other methods than those provide herein including for example, methods other than those embodied in FIG. 1. In some embodiments, the TILs obtained by the present method exhibit an increase in the T-cell repertoire diversity as compared to freshly harvested TILs and/or TILs prepared using methods referred to as process 1C, as exemplified in FIG. 5 and/or FIG. 6. In some embodiments, the TILs obtained in the first expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β).

After dissection or digestion of tumor fragments, for example such as described in Step A of FIG. 1, the resulting cells are cultured in serum containing IL-2 under conditions that favor the growth of TILs over tumor and other cells. In some embodiments, the tumor digests are incubated in 2 mL wells in media comprising inactivated human AB serum with 6000 IU/mL of IL-2. This primary cell population is cultured for a period of days, generally from 3 to 14 days, resulting in a bulk TIL population, generally about 1×10⁸ bulk TIL cells. In some embodiments, this primary cell population is cultured for a period of 7 to 14 days, resulting in a bulk TIL population, generally about 1×10⁸ bulk TIL cells. In some embodiments, this primary cell population is cultured for a period of 10 to 14 days, resulting in a bulk TIL population, generally about 1×10⁸ bulk TIL cells. In some embodiments, this primary cell population is cultured for a period of about 11 days, resulting in a bulk TIL population, generally about 1×10⁸ bulk TIL cells.

In some embodiments, expansion of TILs may be performed using an initial bulk TIL expansion step (for example such as those described in Step B of FIG. 1, which can include processes referred to as pre-REP) as described below and herein, followed by a second expansion (Step D, including processes referred to as rapid expansion protocol (REP) steps) as described below under Step D and herein, followed by optional cryopreservation, and followed by a second Step D (including processes referred to as restimulation REP steps) as described below and herein. The TILs obtained from this process may be optionally characterized for phenotypic characteristics and metabolic parameters as described herein.

In some embodiments where TIL cultures are initiated in 24-well plates, for example, using Costar 24-well cell culture cluster, flat bottom (Corning Incorporated, Corning, NY, each well can be seeded with 1×10⁶ tumor digest cells or one tumor fragment in 2 mL of complete medium (CM) with IL-2 (6000 IU/mL; Chiron Corp., Emeryville, CA). In some embodiments, the tumor fragment is between about 1 mm³ and 10 mm³.

In some embodiments, the first expansion culture medium is referred to as “CM”, an abbreviation for culture media. In some embodiments, CM for Step B consists of RPMI 1640 with GlutaMAX, supplemented with 10% human AB serum, 25 mM Hepes, and 10 mg/mL gentamicin. In some embodiments where cultures are initiated in gas-permeable flasks with a 40 mL capacity and a 10 cm² gas-permeable silicon bottom (for example, G-REX10; Wilson Wolf Manufacturing, New Brighton, MN), each flask was loaded with 10-40×10⁶ viable tumor digest cells or 5-30 tumor fragments in 10-40 mL of CM with IL-2. Both the G-REX10 and 24-well plates were incubated in a humidified incubator at 37° C. in 5% CO₂ and 5 days after culture initiation, half the media was removed and replaced with fresh CM and IL-2 and after day 5, half the media was changed every 2-3 days.

In some embodiments, the culture medium used in the expansion processes disclosed herein is a serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium, CTS™ OpTmizer™ T-Cell Expansion SFM, CTS™ AIM-V Medium, CTS™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (uMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™ OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CST™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (uMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%, 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific). In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM. In some embodiments, the final concentration of 2-mercaptoethanol in the media is 55 μM.

In some embodiments, the defined media described in International PCT Publication No. WO/1998/030679, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, one or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, p, Si⁴⁺, V, Mo⁶, Ni²⁺, Rb⁺, Sn²⁺ and Zr⁴⁺. In some embodiments, the basal cell media is selected from the group consisting of Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., ALbuMAX® I) is about 5000-50,000 mg/L.

In some embodiments, the non-trace element moiety ingredients in the defined medium are present in the concentration ranges listed in the column under the heading “Concentration Range in X Medium” in Table 12 below. In other embodiments, the non-trace element moiety ingredients in the defined medium are present in the final concentrations listed in the column under the heading “A Preferred Embodiment of the X Medium” in Table 12. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 12 below.

TABLE 12
Concentrations of Non-Trace Element Moiety Ingredients

| | A preferred | Concentration | A preferred |
|---|---|---|---|
| | embodiment in | range in 1X | embodiment in 1X |
| | supplement (mg/L) | medium (mg/L) | medium (mg/L) |
| Ingredient | (About) | (About) | (About) |
| Glycine | 150 | 5-200 | 53 |
| L-Histidine | 940 | 5-250 | 183 |
| L-Isoleucine | 3400 | 5-300 | 615 |
| L-Methionine | 90 | 5-200 | 44 |
| L-Phenylalanine | 1800 | 5-400 | 336 |
| L-Proline | 4000 | 1-1000 | 600 |
| L-Hydroxyproline | 100 | 1-45 | 15 |
| L-Serine | 800 | 1-250 | 162 |
| L-Threonine | 2200 | 10-500 | 425 |
| L-Tryptophan | 440 | 2-110 | 82 |
| L-Tyrosine | 77 | 3-175 | 84 |
| L-Valine | 2400 | 5-500 | 454 |
| Thiamine | 33 | 1-20 | 9 |
| Reduced Glutathione | 10 | 1-20 | 1.5 |
| Ascorbic Acid-2- | 330 | 1-200 | 50 |
| PO₄ (Mg Salt) | | | |
| Transferrin | 55 | 1-50 | 8 |
| (iron saturated) | | | |
| Insulin | 100 | 1-100 | 10 |
| Sodium Selenite | 0.07 | 0.000001-0.0001 | 0.00001 |
| AlbuMAX ®I | 83,000 | 5000-50,000 | 12,500 |

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 μM).

In some embodiments, the defined media described in Smith, et al., Clin Transl Immunology, 4(1) 2015 (doi: 10.1038/cti.2014.31) are useful in the present invention. Briefly, RPMI or CTS™ OpTmizer™ was used as the basal cell medium, and supplemented with either 0, 2%, 5%, or 10% CTS™ Immune Cell Serum Replacement.

In some embodiments, the cell medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME or PME; also known as 2-mercaptoethanol, CAS 60-24-2).

After preparation of the tumor fragments, the resulting cells (i.e., fragments) are cultured in serum containing IL-2 under conditions that favor the growth of TILs over tumor and other cells. In some embodiments, the tumor digests are incubated in 2 mL wells in media comprising inactivated human AB serum (or, in some cases, as outlined herein, in the presence of an APC cell population) with 6000 IU/mL of IL-2. This primary cell population is cultured for a period of days, generally from 10 to 14 days, resulting in a bulk TIL population, generally about 1×10⁸ bulk TIL cells. In some embodiments, the growth media during the first expansion comprises IL-2 or a variant thereof. In some embodiments, the IL is recombinant human IL-2 (rhIL-2). In some embodiments the IL-2 stock solution has a specific activity of 20-30×10⁶ IU/mg for a 1 mg vial. In some embodiments the IL-2 stock solution has a specific activity of 20×10⁶ IU/mg for a 1 mg vial. In some embodiments the IL-2 stock solution has a specific activity of 25×10⁶ IU/mg for a 1 mg vial. In some embodiments the IL-2 stock solution has a specific activity of 30×10⁶ IU/mg for a 1 mg vial. In some embodiments, the IL-2 stock solution has a final concentration of 4-8×10⁶ IU/mg of IL-2. In some embodiments, the IL-2 stock solution has a final concentration of 5-7×10⁶ IU/mg of IL-2. In some embodiments, the IL-2 stock solution has a final concentration of 6×10⁶ IU/mg of IL-2. In some embodiments, the IL-2 stock solution is prepare as described in Example 5. In some embodiments, the first expansion culture media comprises about 10,000 IU/mL of IL-2, about 9,000 IU/mL of IL-2, about 8,000 IU/mL of IL-2, about 7,000 IU/mL of IL-2, about 6000 IU/mL of IL-2 or about 5,000 IU/mL of IL-2. In some embodiments, the first expansion culture media comprises about 9,000 IU/mL of IL-2 to about 5,000 IU/mL of IL-2. In some embodiments, the first expansion culture media comprises about 8,000 IU/mL of IL-2 to about 6,000 IU/mL of IL-2. In some embodiments, the first expansion culture media comprises about 7,000 IU/mL of IL-2 to about 6,000 IU/mL of IL-2. In some embodiments, the first expansion culture media comprises about 6,000 IU/mL of IL-2. In some embodiments, the cell culture medium further comprises IL-2. In some embodiments, the cell culture medium comprises about 3000 IU/mL of IL-2. In some embodiments, the cell culture medium further comprises IL-2. In some embodiments, the cell culture medium comprises about 3000 IU/mL of IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In some embodiments, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or about 8000 IU/mL of IL-2.

In some embodiments, first expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the first expansion culture media comprises about 500 IU/mL of IL-15 to about 100 IU/mL of IL-15. In some embodiments, the first expansion culture media comprises about 400 IU/mL of IL-15 to about 100 IU/mL of IL-15. In some embodiments, the first expansion culture media comprises about 300 IU/mL of IL-15 to about 100 IU/mL of IL-15. In some embodiments, the first expansion culture media comprises about 200 IU/mL of IL-15. In some embodiments, the cell culture medium comprises about 180 IU/mL of IL-15. In some embodiments, the cell culture medium further comprises IL-15. In some embodiments, the cell culture medium comprises about 180 IU/mL of IL-15.

In some embodiments, first expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the first expansion culture media comprises about 20 IU/mL of IL-21 to about 0.5 IU/mL of IL-21. In some embodiments, the first expansion culture media comprises about 15 IU/mL of IL-21 to about 0.5 IU/mL of IL-21. In some embodiments, the first expansion culture media comprises about 12 IU/mL of IL-21 to about 0.5 IU/mL of IL-21. In some embodiments, the first expansion culture media comprises about 10 IU/mL of IL-21 to about 0.5 IU/mL of IL-21. In some embodiments, the first expansion culture media comprises about 5 IU/mL of IL-21 to about 1 IU/mL of IL-21. In some embodiments, the first expansion culture media comprises about 2 IU/mL of IL-21. In some embodiments, the cell culture medium comprises about 1 IU/mL of IL-21. In some embodiments, the cell culture medium comprises about 0.5 IU/mL of IL-21. In some embodiments, the cell culture medium further comprises IL-21. In some embodiments, the cell culture medium comprises about 1 IU/mL of IL-21.

In some embodiments, the cell culture medium comprises an anti-CD3 agonist antibody, e.g. OKT-3 antibody. In some embodiments, the cell culture medium comprises about 30 ng/mL of OKT-3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In some embodiments, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium does not comprise OKT-3 antibody. In some embodiments, the OKT-3 antibody is muromonab. See, for example, Table 1.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In some embodiments, the first expansion culture medium is referred to as “CM”, an abbreviation for culture media. In some embodiments, it is referred to as CM1 (culture medium 1). In some embodiments, CM consists of RPMI 1640 with GlutaMAX, supplemented with 10% human AB serum, 25 mM Hepes, and 10 mg/mL gentamicin. In some embodiments where cultures are initiated in gas-permeable flasks with a 40 mL capacity and a 10 cm² gas-permeable silicon bottom (for example, G-REX10; Wilson Wolf Manufacturing, New Brighton, MN), each flask was loaded with 10-40×10⁶ viable tumor digest cells or 5-30 tumor fragments in 10-40 mL of CM with IL-2. Both the G-REX10 and 24-well plates were incubated in a humidified incubator at 37° C. in 5% CO₂ and 5 days after culture initiation, half the media was removed and replaced with fresh CM and IL-2 and after day 5, half the media was changed every 2-3 days. In some embodiments, the CM is the CM1 described in the Examples, see, Example 1. In some embodiments, the first expansion occurs in an initial cell culture medium or a first cell culture medium. In some embodiments, the initial cell culture medium or the first cell culture medium comprises IL-2.

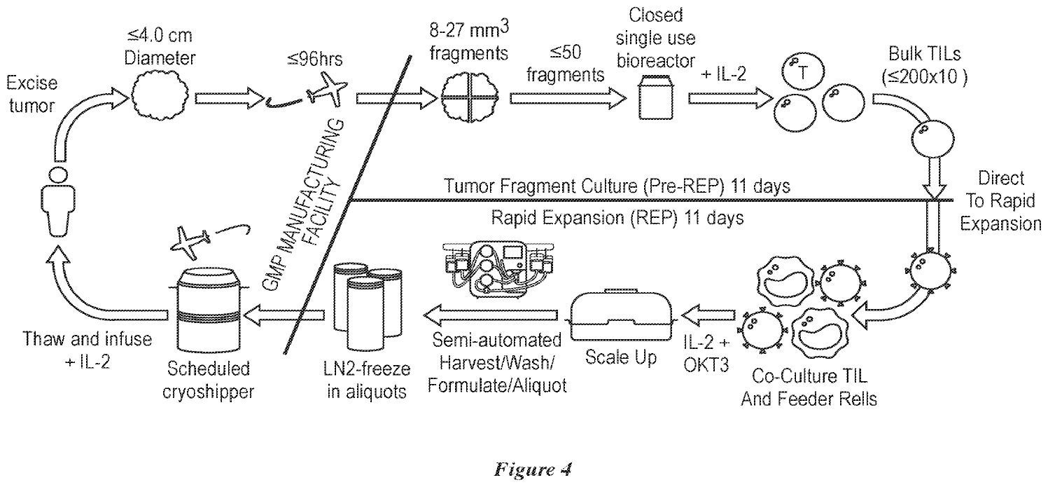

In some embodiments, the first expansion (including processes such as for example those described in Step B of FIG. 1, which can include those sometimes referred to as the pre-REP) process is shortened to 3-14 days, as discussed in the examples and figures. In some embodiments, the first expansion (including processes such as for example those described in Step B of FIG. 1, which can include those sometimes referred to as the pre-REP) is shortened to 7 to 14 days, as discussed in the Examples and shown in FIGS. 4 and 5, as well as including for example, an expansion as described in Step B of FIG. 1. In some embodiments, the first expansion of Step B is shortened to 10-14 days. In some embodiments, the first expansion is shortened to 11 days, as discussed in, for example, an expansion as described in Step B of FIG. 1.

In some embodiments, the first TIL expansion can proceed for 1 day, 2 days, 3 days, 4 days, 5 days, 6 days, 7 days, 8 days, 9 days, 10 days, 11 days, 12 days, 13 days, or 14 days. In some embodiments, the first TIL expansion can proceed for 1 day to 14 days. In some embodiments, the first TIL expansion can proceed for 2 days to 14 days. In some embodiments, the first TIL expansion can proceed for 3 days to 14 days. In some embodiments, the first TIL expansion can proceed for 4 days to 14 days. In some embodiments, the first TIL expansion can proceed for 5 days to 14 days. In some embodiments, the first TIL expansion can proceed for 6 days to 14 days. In some embodiments, the first TIL expansion can proceed for 7 days to 14 days. In some embodiments, the first TIL expansion can proceed for 8 days to 14 days. In some embodiments, the first TIL expansion can proceed for 9 days to 14 days. In some embodiments, the first TIL expansion can proceed for 10 days to 14 days. In some embodiments, the first TIL expansion can proceed for 11 days to 14 days. In some embodiments, the first TIL expansion can proceed for 12 days to 14 days. In some embodiments, the first TIL expansion can proceed for 13 days to 14 days. In some embodiments, the first TIL expansion can proceed for 14 days. In some embodiments, the first TIL expansion can proceed for 1 day to 11 days. In some embodiments, the first TIL expansion can proceed for 2 days to 11 days. In some embodiments, the first TIL expansion can proceed for 3 days to 11 days. In some embodiments, the first TIL expansion can proceed for 4 days to 11 days. In some embodiments, the first TIL expansion can proceed for 5 days to 11 days. In some embodiments, the first TIL expansion can proceed for 6 days to 11 days. In some embodiments, the first TIL expansion can proceed for 7 days to 11 days. In some embodiments, the first TIL expansion can proceed for 8 days to 11 days. In some embodiments, the first TIL expansion can proceed for 9 days to 11 days. In some embodiments, the first TIL expansion can proceed for 10 days to 11 days. In some embodiments, the first TIL expansion can proceed for 11 days.

In some embodiments, a combination of IL-2, IL-7, IL-15, and/or IL-21 are employed as a combination during the first expansion. In some embodiments, IL-2, IL-7, IL-15, and/or IL-21 as well as any combinations thereof can be included during the first expansion, including for example during a Step B processes according to FIG. 1, as well as described herein. In some embodiments, a combination of IL-2, IL-15, and IL-21 are employed as a combination during the first expansion. In some embodiments, IL-2, IL-15, and IL-21 as well as any combinations thereof can be included during Step B processes according to FIG. 1 and as described herein.

In some embodiments, the first expansion (including processes referred to as the pre-REP; for example, Step B according to FIG. 1) process is shortened to 3 to 14 days, as discussed in the examples and figures. In some embodiments, the first expansion of Step B is shortened to 7 to 14 days. In some embodiments, the first expansion of Step B is shortened to 10 to 14 days. In some embodiments, the first expansion is shortened to 11 days.

In some embodiments, the first expansion, for example, Step B according to FIG. 1, is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

### 1. Cytokines and Other Additives

The expansion methods described herein generally use culture media with high doses of a cytokine, in particular IL-2, as is known in the art.

Alternatively, using combinations of cytokines for the rapid expansion and or second expansion of TILs is additionally possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21 and IL-2, or IL-15 and IL-21, with the latter finding particular use in many embodiments. The use of combinations of cytokines specifically favors the generation of lymphocytes, and in particular T-cells as described therein.

In some embodiments, Step B may also include the addition of OKT-3 antibody or muromonab to the culture media, as described elsewhere herein. In some embodiments, Step B may also include the addition of a 4-1BB agonist to the culture media, as described elsewhere herein. In some embodiments, Step B may also include the addition of an OX-40 agonist to the culture media, as described elsewhere herein. In other embodiments, additives such as peroxisome proliferator-activated receptor gamma coactivator I-alphan agonists, including proliferator-activated receptor (PPAR)-gamma agonists such as a thiazolidinedione compound, may be used in the culture media during Step B, as described in U.S. Patent Application Publication No. US 2019/0307796 A1, the disclosure of which is incorporated by reference herein.

### C. STEP C: First Expansion to Second Expansion Transition

In some cases, the bulk TIL population obtained from the first expansion, including for example the TIL population obtained from for example, Step B as indicated in FIG. 1, can be cryopreserved immediately, using the protocols discussed herein below. Alternatively, the TIL population obtained from the first expansion, referred to as the second TIL population, can be subjected to a second expansion (which can include expansions sometimes referred to as REP) and then cryopreserved as discussed below. Similarly, in the case where genetically modified TILs will be used in therapy, the first TIL population (sometimes referred to as the bulk TIL population) or the second TIL population (which can in some embodiments include populations referred to as the REP TIL populations) can be subjected to genetic modifications for suitable treatments prior to expansion or after the first expansion and prior to the second expansion.

In some embodiments, the TILs obtained from the first expansion (for example, from Step B as indicated in FIG. 1) are stored until phenotyped for selection. In some embodiments, the TILs obtained from the first expansion (for example, from Step B as indicated in FIG. 1) are not stored and proceed directly to the second expansion. In some embodiments, the TILs obtained from the first expansion are not cryopreserved after the first expansion and prior to the second expansion. In some embodiments, the transition from the first expansion to the second expansion occurs at about 3 days, 4, days, 5 days, 6 days, 7 days, 8 days, 9 days, 10 days, 11 days, 12 days, 13 days, or 14 days from when fragmentation occurs. In some embodiments, the transition from the first expansion to the second expansion occurs at about 3 days to 14 days from when fragmentation occurs. In some embodiments, the transition from the first expansion to the second expansion occurs at about 4 days to 14 days from when fragmentation occurs. In some embodiments, the transition from the first expansion to the second expansion occurs at about 4 days to 10 days from when fragmentation occurs. In some embodiments, the transition from the first expansion to the second expansion occurs at about 7 days to 14 days from when fragmentation occurs. In some embodiments, the transition from the first expansion to the second expansion occurs at about 14 days from when fragmentation occurs.

In some embodiments, the transition from the first expansion to the second expansion occurs at 1 day, 2 days, 3 days, 4 days, 5 days, 6 days, 7 days, 8 days, 9 days, 10 days, 11 days, 12 days, 13 days, or 14 days from when fragmentation occurs. In some embodiments, the transition from the first expansion to the second expansion occurs 1 day to 14 days from when fragmentation occurs. In some embodiments, the first TIL expansion can proceed for 2 days to 14 days. In some embodiments, the transition from the first expansion to the second expansion occurs 3 days to 14 days from when fragmentation occurs. In some embodiments, the transition from the first expansion to the second expansion occurs 4 days to 14 days from when fragmentation occurs. In some embodiments, the transition from the first expansion to the second expansion occurs 5 days to 14 days from when fragmentation occurs. In some embodiments, the transition from the first expansion to the second expansion occurs 6 days to 14 days from when fragmentation occurs. In some embodiments, the transition from the first expansion to the second expansion occurs 7 days to 14 days from when fragmentation occurs. In some embodiments, the transition from the first expansion to the second expansion occurs 8 days to 14 days from when fragmentation occurs. In some embodiments, the transition from the first expansion to the second expansion occurs 9 days to 14 days from when fragmentation occurs. In some embodiments, the transition from the first expansion to the second expansion occurs 10 days to 14 days from when fragmentation occurs. In some embodiments, the transition from the first expansion to the second expansion occurs 11 days to 14 days from when fragmentation occurs. In some embodiments, the transition from the first expansion to the second expansion occurs 12 days to 14 days from when fragmentation occurs. In some embodiments, the transition from the first expansion to the second expansion occurs 13 days to 14 days from when fragmentation occurs. In some embodiments, the transition from the first expansion to the second expansion occurs 14 days from when fragmentation occurs. In some embodiments, the transition from the first expansion to the second expansion occurs 1 day to 11 days from when fragmentation occurs. In some embodiments, the transition from the first expansion to the second expansion occurs 2 days to 11 days from when fragmentation occurs. In some embodiments, the transition from the first expansion to the second expansion occurs 3 days to 11 days from when fragmentation occurs. In some embodiments, the transition from the first expansion to the second expansion occurs 4 days to 11 days from when fragmentation occurs. In some embodiments, the transition from the first expansion to the second expansion occurs 5 days to 11 days from when fragmentation occurs. In some embodiments, the transition from the first expansion to the second expansion occurs 6 days to 11 days from when fragmentation occurs. In some embodiments, the transition from the first expansion to the second expansion occurs 7 days to 11 days from when fragmentation occurs. In some embodiments, the transition from the first expansion to the second expansion occurs 8 days to 11 days from when fragmentation occurs. In some embodiments, the transition from the first expansion to the second expansion occurs 9 days to 11 days from when fragmentation occurs. In some embodiments, the transition from the first expansion to the second expansion occurs 10 days to 11 days from when fragmentation occurs. In some embodiments, the transition from the first expansion to the second expansion occurs 11 days from when fragmentation occurs.

In some embodiments, the TILs are not stored after the first expansion and prior to the second expansion, and the TILs proceed directly to the second expansion (for example, in some embodiments, there is no storage during the transition from Step B to Step D as shown in FIG. 1). In some embodiments, the transition occurs in closed system, as described herein. In some embodiments, the TILs from the first expansion, the second population of TILs, proceeds directly into the second expansion with no transition period.

In some embodiments, the transition from the first expansion to the second expansion, for example, Step C according to FIG. 1, is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100 bioreactor. In some embodiments, the closed system bioreactor is a single bioreactor.

### D. STEP D: Second Expansion

In some embodiments, the TIL cell population is expanded in number after harvest and initial bulk processing for example, after Step A and Step B, and the transition referred to as Step C, as indicated in FIG. 1). This further expansion is referred to herein as the second expansion, which can include expansion processes generally referred to in the art as a rapid expansion process (REP); as well as processes as indicated in Step D of FIG. 1. The second expansion is generally accomplished using a culture media comprising a number of components, including feeder cells, a cytokine source, and an anti-CD3 antibody, in a gas-permeable container.

In some embodiments, the second expansion or second TIL expansion (which can include expansions sometimes referred to as REP; as well as processes as indicated in Step D of FIG. 1) of TIL can be performed using any TIL flasks or containers known by those of skill in the art. In some embodiments, the second TIL expansion can proceed for 7 days, 8 days, 9 days, 10 days, 11 days, 12 days, 13 days, or 14 days. In some embodiments, the second TIL expansion can proceed for about 7 days to about 14 days. In some embodiments, the second TIL expansion can proceed for about 8 days to about 14 days. In some embodiments, the second TIL expansion can proceed for about 9 days to about 14 days. In some embodiments, the second TIL expansion can proceed for about 10 days to about 14 days. In some embodiments, the second TIL expansion can proceed for about 11 days to about 14 days. In some embodiments, the second TIL expansion can proceed for about 12 days to about 14 days. In some embodiments, the second TIL expansion can proceed for about 13 days to about 14 days. In some embodiments, the second TIL expansion can proceed for about 14 days.

In some embodiments, the second expansion can be performed in a gas permeable container using the methods of the present disclosure (including for example, expansions referred to as REP; as well as processes as indicated in Step D of FIG. 1). For example, TILs can be rapidly expanded using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/mL of OKT3, a mouse monoclonal anti-CD3 antibody (commercially available from Ortho-McNeil, Raritan, NJ or Miltenyi Biotech, Auburn, CA) or UHCT-1 (commercially available from BioLegend, San Diego, CA, USA). TILs can be expanded to induce further stimulation of the TILs in vitro by including one or more antigens during the second expansion, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2. In some embodiments, the re-stimulation occurs as part of the second expansion. In some embodiments, the second expansion occurs in the presence of irradiated, autologous lymphocytes or with irradiated HLA-A2+ allogeneic lymphocytes and IL-2.

In some embodiments, the cell culture medium further comprises IL-2. In some embodiments, the cell culture medium comprises about 3000 IU/mL of IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In some embodiments, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In some embodiments, the cell culture medium comprises OKT-3 antibody. In some embodiments, the cell culture medium comprises about 30 ng/mL of OKT-3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In some embodiments, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium does not comprise OKT-3 antibody. In some embodiments, the OKT-3 antibody is muromonab.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In some embodiments, a combination of IL-2, IL-7, IL-15, and/or IL-21 are employed as a combination during the second expansion. In some embodiments, IL-2, IL-7, IL-15, and/or IL-21 as well as any combinations thereof can be included during the second expansion, including for example during a Step D processes according to FIG. 1, as well as described herein. In some embodiments, a combination of IL-2, IL-15, and IL-21 are employed as a combination during the second expansion. In some embodiments, IL-2, IL-15, and IL-21 as well as any combinations thereof can be included during Step D processes according to FIG. 1 and as described herein.

In some embodiments, the second expansion can be conducted in a supplemented cell culture medium comprising IL-2, OKT-3, antigen-presenting feeder cells, and optionally a TNFRSF agonist. In some embodiments, the second expansion occurs in a supplemented cell culture medium. In some embodiments, the supplemented cell culture medium comprises IL-2, OKT-3, and antigen-presenting feeder cells. In some embodiments, the second cell culture medium comprises IL-2, OKT-3, and antigen-presenting cells (APCs; also referred to as antigen-presenting feeder cells). In some embodiments, the second expansion occurs in a cell culture medium comprising IL-2, OKT-3, and antigen-presenting feeder cells (i.e., antigen presenting cells).

In some embodiments, the second expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the second expansion culture media comprises about 500 IU/mL of IL-15 to about 100 IU/mL of IL-15. In some embodiments, the second expansion culture media comprises about 400 IU/mL of IL-15 to about 100 IU/mL of IL-15. In some embodiments, the second expansion culture media comprises about 300 IU/mL of IL-15 to about 100 IU/mL of IL-15. In some embodiments, the second expansion culture media comprises about 200 IU/mL of IL-15. In some embodiments, the cell culture medium comprises about 180 IU/mL of IL-15. In some embodiments, the cell culture medium further comprises IL-15. In some embodiments, the cell culture medium comprises about 180 IU/mL of IL-15.

In some embodiments, the second expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the second expansion culture media comprises about 20 IU/mL of IL-21 to about 0.5 IU/mL of IL-21. In some embodiments, the second expansion culture media comprises about 15 IU/mL of IL-21 to about 0.5 IU/mL of IL-21. In some embodiments, the second expansion culture media comprises about 12 IU/mL of IL-21 to about 0.5 IU/mL of IL-21. In some embodiments, the second expansion culture media comprises about 10 IU/mL of IL-21 to about 0.5 IU/mL of IL-21. In some embodiments, the second expansion culture media comprises about 5 IU/mL of IL-21 to about 1 IU/mL of IL-21. In some embodiments, the second expansion culture media comprises about 2 IU/mL of IL-21. In some embodiments, the cell culture medium comprises about 1 IU/mL of IL-21. In some embodiments, the cell culture medium comprises about 0.5 IU/mL of IL-21. In some embodiments, the cell culture medium further comprises IL-21. In some embodiments, the cell culture medium comprises about 1 IU/mL of IL-21.

In some embodiments the antigen-presenting feeder cells (APCs) are PBMCs. In some embodiments, the ratio of TILs to PBMCs and/or antigen-presenting cells in the rapid expansion and/or the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to PBMCs in the rapid expansion and/or the second expansion is between 1 to 50 and 1 to 300. In some embodiments, the ratio of TILs to PBMCs in the rapid expansion and/or the second expansion is between 1 to 100 and 1 to 200.

In some embodiments, REP and/or the second expansion is performed in flasks with the bulk TILs being mixed with a 100- or 200-fold excess of inactivated feeder cells, 30 mg/mL OKT3 anti-CD3 antibody and 3000 IU/mL IL-2 in 150 mL media. Media replacement is done (generally 2/3 media replacement via respiration with fresh media) until the cells are transferred to an alternative growth chamber. Alternative growth chambers include G-REX flasks and gas permeable containers as more fully discussed below.

In some embodiments, the second expansion (which can include processes referred to as the REP process) is shortened to 7-14 days, as discussed in the examples and figures. In some embodiments, the second expansion is shortened to 11 days.

In some embodiments, REP and/or the second expansion may be performed using T-175 flasks and gas permeable bags as previously described (Tran, et al., J Immunother. 2008, 31, 742-51; Dudley, et al., J Immunother. 2003, 26, 332-42) or gas permeable cultureware (G-REX flasks). In some embodiments, the second expansion (including expansions referred to as rapid expansions) is performed in T-175 flasks, and about 1×10⁶ TILs suspended in 150 mL of media may be added to each T-175 flask. The TILs may be cultured in a 1 to 1 mixture of CM and AIM-V medium, supplemented with 3000 IU per mL of IL-2 and 30 ng per mL of anti-CD3. The T-175 flasks may be incubated at 37° C. in 5% CO₂. Half the media may be exchanged on day 5 using 50/50 medium with 3000 IU per mL of IL-2. In some embodiments, on day 7 cells from two T-175 flasks may be combined in a 3 L bag and 300 mL of AIM V with 5% human AB serum and 3000 IU per mL of IL-2 was added to the 300 mL of TIL suspension. The number of cells in each bag was counted every day or two and fresh media was added to keep the cell count between 0.5 and 2.0×10⁶ cells/mL.

In some embodiments, the second expansion (which can include expansions referred to as REP, as well as those referred to in Step D of FIG. 1) may be performed in 500 mL capacity gas permeable flasks with 100 cm gas-permeable silicon bottoms (G-REX-100, commercially available from Wilson Wolf Manufacturing Corporation, New Brighton, MN, USA), 5×10⁶ or 10×10⁶ TIL may be cultured with PBMCs in 400 mL of 50/50 medium, supplemented with 5% human AB serum, 3000 IU per mL of IL-2 and 30 ng per mL of anti-CD3 (OKT3). The G-REX-100 flasks may be incubated at 37° C. in 5% CO₂. On day 5, 250 mL of supernatant may be removed and placed into centrifuge bottles and centrifuged at 1500 rpm (491×g) for 10 minutes. The TIL pellets may be re-suspended with 150 mL of fresh medium with 5% human AB serum, 3000 IU per mL of IL-2, and added back to the original G-REX-100 flasks. When TIL are expanded serially in G-REX-100 flasks, on day 7 the TIL in each G-REX-100 may be suspended in the 300 mL of media present in each flask and the cell suspension may be divided into 3 100 mL aliquots that may be used to seed 3 G-REX-100 flasks. Then 150 mL of AIM-V with 5% human AB serum and 3000 IU per mL of IL-2 may be added to each flask. The G-REX-100 flasks may be incubated at 37° C. in 5% CO₂ and after 4 days 150 mL of AIM-V with 3000 IU per mL of IL-2 may be added to each G-REX-100 flask. The cells may be harvested on day 14 of culture.

In some embodiments, the second expansion (including expansions referred to as REP) is performed in flasks with the bulk TILs being mixed with a 100- or 200-fold excess of inactivated feeder cells, 30 mg/mL OKT3 anti-CD3 antibody and 3000 IU/mL IL-2 in 150 mL media. In some embodiments, media replacement is done until the cells are transferred to an alternative growth chamber. In some embodiments, 2/3 of the media is replaced by respiration with fresh media. In some embodiments, alternative growth chambers include G-REX flasks and gas permeable containers as more fully discussed below.

In some embodiments, the second expansion (including expansions referred to as REP) is performed and further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

Optionally, a cell viability assay can be performed after the second expansion (including expansions referred to as the REP expansion), using standard assays known in the art. For example, a trypan blue exclusion assay can be done on a sample of the bulk TILs, which selectively labels dead cells and allows a viability assessment. In some embodiments, TIL samples can be counted and viability determined using a Cellometer K2 automated cell counter (Nexcelom Bioscience, Lawrence, MA). In some embodiments, viability is determined according to the standard Cellometer K2 Image Cytometer Automatic Cell Counter protocol.

In some embodiments, the second expansion (including expansions referred to as REP) of TIL can be performed using T-175 flasks and gas-permeable bags as previously described (Tran, et al., 2008, J Immunother., 31, 742-751, and Dudley, et al. 2003, J Immunother., 26, 332-342) or gas-permeable G-REX flasks. In some embodiments, the second expansion is performed using flasks. In some embodiments, the second expansion is performed using gas-permeable G-REX flasks. In some embodiments, the second expansion is performed in T-175 flasks, and about 1×10⁶ TIL are suspended in about 150 mL of media and this is added to each T-175 flask. The TIL are cultured with irradiated (50 Gy) allogeneic PBMC as “feeder” cells at a ratio of 1 to 100 and the cells were cultured in a 1 to 1 mixture of CM and AIM-V medium (50/50 medium), supplemented with 3000 IU/mL of IL-2 and 30 ng/mL of anti-CD3. The T-175 flasks are incubated at 37° C. in 5% CO₂. In some embodiments, half the media is changed on day 5 using 50/50 medium with 3000 IU/mL of IL-2. In some embodiments, on day 7, cells from 2 T-175 flasks are combined in a 3 L bag and 300 mL of AIM-V with 5% human AB serum and 3000 IU/mL of IL-2 is added to the 300 mL of TIL suspension. The number of cells in each bag can be counted every day or two and fresh media can be added to keep the cell count between about 0.5 and about 2.0×10⁶ cells/mL.

In some embodiments, the second expansion (including expansions referred to as REP) are performed in 500 mL capacity flasks with 100 cm² gas-permeable silicon bottoms (G-REX-100, Wilson Wolf) about 5×10⁶ or 10×10⁶ TIL are cultured with irradiated allogeneic PBMC at a ratio of 1 to 100 in 400 mL of 50/50 medium, supplemented with 3000 IU/mL of IL-2 and 30 ng/mL of anti-CD3. The G-REX-100 flasks are incubated at 37° C. in 5% CO₂. In some embodiments, on day 5, 250 mL of supernatant is removed and placed into centrifuge bottles and centrifuged at 1500 rpm (491 g) for 10 minutes. The TIL pellets can then be resuspended with 150 mL of fresh 50/50 medium with 3000 IU/mL of IL-2 and added back to the original G-REX-100 flasks. In some embodiments where TILs are expanded serially in G-REX-100 flasks, on day 7 the TIL in each G-REX-100 are suspended in the 300 mL of media present in each flask and the cell suspension was divided into three 100 mL aliquots that are used to seed 3 G-REX-100 flasks. Then 150 mL of AIM-V with 5% human AB serum and 3000 IU/mL of IL-2 is added to each flask. The G-REX-100 flasks are incubated at 37° C. in 5% CO₂ and after 4 days 150 mL of AIM-V with 3000 IU/mL of IL-2 is added to each G-REX-100 flask. The cells are harvested on day 14 of culture.

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained in the second expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β).

In some embodiments, the second expansion culture medium (e.g., sometimes referred to as CM2 or the second cell culture medium), comprises IL-2, OKT-3, as well as the antigen-presenting feeder cells (APCs), as discussed in more detail below.

In some embodiments, the culture medium used in the expansion processes disclosed herein is a serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium, CTS™ OpTmizer™ T-Cell Expansion SFM, CTS™ AIM-V Medium, CTS™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™ OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CST™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%, 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific). In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM. In some embodiments, the final concentration of 2-mercaptoethanol in the media is 55 μM.

In some embodiments, the defined media described in International PCT Publication No. WO/1998/030679, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, one or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, p, Si⁴⁺, V⁵⁺, Mo⁶⁺, Ni²⁺, Rb⁺, Sn²⁺ and Zr⁴⁺. In some embodiments, the basal cell media is selected from the group consisting of Dulbecco's Modified Eagle's Medium (DMEM), Minimal Essential Medium (MEM), Basal Medium Eagle (BME), RPMI 1640, F-10, F-12, Minimal Essential Medium (uMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., AlbuMAX® I) is about 5000-50,000 mg/L.

In some embodiments, the non-trace element moiety ingredients in the defined medium are present in the concentration ranges listed in the column under the heading “Concentration Range in 1× Medium” in Table 12. In other embodiments, the non-trace element moiety ingredients in the defined medium are present in the final concentrations listed in the column under the heading “A Preferred Embodiment of the 1× Medium” in Table 12. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 12.

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 M).

In some embodiments, the defined media described in Smith, et al., Clin Transl Immunology, 4(1) 2015 (doi: 10.1038/cti.2014.31) are useful in the present invention. Briefly, RPMI or CTS™ OpTmizer™ was used as the basal cell medium, and supplemented with either 0, 2%, 5%, or 10% CTS™ Immune Cell Serum Replacement.

In some embodiments, the cell medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME or OME; also known as 2-mercaptoethanol, CAS 60-24-2).

In some embodiments, the second expansion, for example, Step D according to FIG. 1, is performed in a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

In some embodiments, the step of rapid or second expansion is split into a plurality of steps to achieve a scaling up of the culture by: (a) performing the rapid or second expansion by culturing TILs in a small scale culture in a first container, e.g., a G-REX-100 MCS container, for a period of about 3 to 7 days, and then (b) effecting the transfer of the TILs in the small scale culture to a second container larger than the first container, e.g., a G-REX-500-MCS container, and culturing the TILs from the small scale culture in a larger scale culture in the second container for a period of about 4 to 7 days.

In some embodiments, the step of rapid or second expansion is split into a plurality of steps to achieve a scaling out of the culture by: (a) performing the rapid or second expansion by culturing TILs in a first small scale culture in a first container, e.g., a G-REX-100 MCS container, for a period of about 3 to 7 days, and then (b) effecting the transfer and apportioning of the TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the TILs from first small scale culture transferred to such second container is cultured in a second small scale culture for a period of about 4 to 7 days.

In some embodiments, the first small scale TIL culture is apportioned into a plurality of about 2 to 5 subpopulations of TILs.

In some embodiments, the step of rapid or second expansion is split into a plurality of steps to achieve a scaling out and scaling up of the culture by: (a) performing the rapid or second expansion by culturing TILs in a small scale culture in a first container, e.g., a G-REX-100 MCS container, for a period of about 3 to 7 days, and then (b) effecting the transfer and apportioning of the TILs from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX-500MCS containers, wherein in each second container the portion of the TILs from the small scale culture transferred to such second container is cultured in a larger scale culture for a period of about 4 to 7 days.

In some embodiments, the step of rapid or second expansion is split into a plurality of steps to achieve a scaling out and scaling up of the culture by: (a) performing the rapid or second expansion by culturing TILs in a small scale culture in a first container, e.g., a G-REX-100 MCS container, for a period of about 5 days, and then (b) effecting the transfer and apportioning of the TILs from the small scale culture into and amongst 2, 3 or 4 second containers that are larger in size than the first container, e.g., G-REX-500 MCS containers, wherein in each second container the portion of the TILs from the small scale culture transferred to such second container is cultured in a larger scale culture for a period of about 6 days.

In some embodiments, upon the splitting of the rapid or second expansion, each second container comprises at least 108 TILs. In some embodiments, upon the splitting of the rapid or second expansion, each second container comprises at least 108 TILs, at least 109 TILs, or at least 10¹⁰ TILs. In one exemplary embodiment, each second container comprises at least 10¹⁰ TILs.

In some embodiments, the first small scale TIL culture is apportioned into a plurality of subpopulations. In some embodiments, the first small scale TIL culture is apportioned into a plurality of about 2 to 5 subpopulations. In some embodiments, the first small scale TIL culture is apportioned into a plurality of about 2, 3, 4, or 5 subpopulations.

In some embodiments, after the completion of the rapid or second expansion, the plurality of subpopulations comprises a therapeutically effective amount of TILs. In some embodiments, after the completion of the rapid or second expansion, one or more subpopulations of TILs are pooled together to produce a therapeutically effective amount of TILs. In some embodiments, after the completion of the rapid expansion, each subpopulation of TILs comprises a therapeutically effective amount of TILs.

In some embodiments, the rapid or second expansion is performed for a period of about 3 to 7 days before being split into a plurality of steps. In some embodiments, the splitting of the rapid or second expansion occurs at about day 3, day 4, day 5, day 6, or day 7 after the initiation of the rapid or second expansion.

In some embodiments, the splitting of the rapid or second expansion occurs at about day 7, day 8, day 9, day 10, day 11, day 12, day 13, day 14, day 15, or day 16 day 17, or day 18 after the initiation of the first expansion (i.e., pre-REP expansion). In one exemplary embodiment, the splitting of the rapid or second expansion occurs at about day 16 after the initiation of the first expansion.

In some embodiments, the rapid or second expansion is further performed for a period of about 7 to 11 days after the splitting. In some embodiments, the rapid or second expansion is further performed for a period of about 5 days, 6 days, 7 days, 8 days, 9 days, 10 days, or 11 days after the splitting.

In some embodiments, the cell culture medium used for the rapid or second expansion before the splitting comprises the same components as the cell culture medium used for the rapid or second expansion after the splitting. In some embodiments, the cell culture medium used for the rapid or second expansion before the splitting comprises different components from the cell culture medium used for the rapid or second expansion after the splitting.

In some embodiments, the cell culture medium used for the rapid or second expansion before the splitting comprises IL-2, optionally OKT-3 and further optionally APCs. In some embodiments, the cell culture medium used for the rapid or second expansion before the splitting comprises IL-2, OKT-3, and further optionally APCs. In some embodiments, the cell culture medium used for the rapid or second expansion before the splitting comprises IL-2, OKT-3 and APCs.

In some embodiments, the cell culture medium used for the rapid or second expansion before the splitting is generated by supplementing the cell culture medium in the first expansion with fresh culture medium comprising IL-2, optionally OKT-3 and further optionally APCs. In some embodiments, the cell culture medium used for the rapid or second expansion before the splitting is generated by supplementing the cell culture medium in the first expansion with fresh culture medium comprising IL-2, OKT-3 and APCs. In some embodiments, the cell culture medium used for the rapid or second expansion before the splitting is generated by replacing the cell culture medium in the first expansion with fresh cell culture medium comprising IL-2, optionally OKT-3 and further optionally APCs. In some embodiments, the cell culture medium used for the rapid or second expansion before the splitting is generated by replacing the cell culture medium in the first expansion with fresh cell culture medium comprising IL-2, OKT-3 and APCs.

In some embodiments, the cell culture medium used for the rapid or second expansion after the splitting comprises IL-2, and optionally OKT-3. In some embodiments, the cell culture medium used for the rapid or second expansion after the splitting comprises IL-2, and OKT-3. In some embodiments, the cell culture medium used for the rapid or second expansion after the splitting is generated by replacing the cell culture medium used for the rapid or second expansion before the splitting with fresh culture medium comprising IL-2 and optionally OKT-3. In some embodiments, the cell culture medium used for the rapid or second expansion after the splitting is generated by replacing the cell culture medium used for the rapid or second expansion before the splitting with fresh culture medium comprising IL-2 and OKT-3.

In some embodiments, the splitting of the rapid expansion occurs in a closed system.

In some embodiments, the scaling up of the TIL culture during the rapid or second expansion comprises adding fresh cell culture medium to the TIL culture (also referred to as feeding the TILs). In some embodiments, the feeding comprises adding fresh cell culture medium to the TIL culture frequently. In some embodiments, the feeding comprises adding fresh cell culture medium to the TIL culture at a regular interval. In some embodiments, the fresh cell culture medium is supplied to the TILs via a constant flow. In some embodiments, an automated cell expansion system such as Xuri W25 is used for the rapid expansion and feeding.

### 1. Feeder Cells and Antigen Presenting Cells

In some embodiments, the second expansion procedures described herein (for example including expansion such as those described in Step D from FIG. 1, as well as those referred to as REP) require an excess of feeder cells during REP TIL expansion and/or during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogeneic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and accepted for use in the TIL expansion procedures described herein if the total number of viable cells on day 14 is less than the initial viable cell number put into culture on day 0 of the REP and/or day 0 of the second expansion (i.e., the start day of the second expansion).

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/mL OKT3 antibody and 3000 IU/mL IL-2.

In some embodiments, PBMCs are considered replication incompetent and accepted for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 5-60 ng/mL OKT3 antibody and 1000-6000 IU/mL IL-2. In some embodiments, the PBMCs are cultured in the presence of 10-50 ng/mL OKT3 antibody and 2000-5000 IU/mL IL-2. In some embodiments, the PBMCs are cultured in the presence of 20-40 ng/mL OKT3 antibody and 2000-4000 IU/mL IL-2. In some embodiments, the PBMCs are cultured in the presence of 25-35 ng/mL OKT3 antibody and 2500-3500 IU/mL IL-2.

In some embodiments, the antigen-presenting feeder cells are PBMCs. In some embodiments, the antigen-presenting feeder cells are artificial antigen-presenting feeder cells. In some embodiments, the ratio of TILs to antigen-presenting feeder cells in the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to antigen-presenting feeder cells in the second expansion is between 1 to 50 and 1 to 300. In some embodiments, the ratio of TILs to antigen-presenting feeder cells in the second expansion is between 1 to 100 and 1 to 200.

In some embodiments, the second expansion procedures described herein require a ratio of about 2.5×10⁹ feeder cells to about 100×10⁶ TIL. In other embodiments, the second expansion procedures described herein require a ratio of about 2.5×10⁹ feeder cells to about 50×10⁶ TIL. In yet other embodiments, the second expansion procedures described herein require about 2.5×10⁹ feeder cells to about 25×10⁶ TIL.

In some embodiments, the second expansion procedures described herein require an excess of feeder cells during the second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation. In some embodiments, artificial antigen-presenting (aAPC) cells are used in place of PBMCs.

In general, the allogeneic PBMCs are inactivated, either via irradiation or heat treatment, and used in the TIL expansion procedures described herein, including the exemplary procedures described in the figures and examples.

In some embodiments, artificial antigen presenting cells are used in the second expansion as a replacement for, or in combination with, PBMCs.

### 2. Cytokines and Other Additives

The expansion methods described herein generally use culture media with high doses of a cytokine, in particular IL-2, as is known in the art.

Alternatively, using combinations of cytokines for the rapid expansion and or second expansion of TILs is additionally possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21 and IL-2, IL-15 and IL-21, with the latter finding particular use in many embodiments. The use of combinations of cytokines specifically favors the generation of lymphocytes, and in particular T-cells as described therein.

In some embodiments, Step D may also include the addition of OKT-3 antibody or muromonab to the culture media, as described elsewhere herein. In some embodiments, Step D may also include the addition of a 4-1BB agonist to the culture media, as described elsewhere herein. In some embodiments, Step D may also include the addition of an OX-40 agonist to the culture media, as described elsewhere herein. In addition, additives such as peroxisome proliferator-activated receptor gamma coactivator I-alpha agonists, including proliferator-activated receptor (PPAR)-gamma agonists such as a thiazolidinedione compound, may be used in the culture media during Step D, as described in U.S. Patent Application Publication No. US 2019/0307796 A1, the disclosure of which is incorporated by reference herein.

### E. STEP E: Harvest TILs

After the second expansion step, cells can be harvested. In some embodiments the TILs are harvested after one, two, three, four or more expansion steps, for example as provided in FIG. 1. In some embodiments the TILs are harvested after two expansion steps, for example as provided in FIG. 1.

TILs can be harvested in any appropriate and sterile manner, including for example by centrifugation. Methods for TIL harvesting are well known in the art and any such know methods can be employed with the present process. In some embodiments, TILs are harvested using an automated system.

Cell harvesters and/or cell processing systems are commercially available from a variety of sources, including, for example, Fresenius Kabi, Tomtec Life Science, Perkin Elmer, and Inotech Biosystems International, Inc. Any cell based harvester can be employed with the present methods. In some embodiments, the cell harvester and/or cell processing systems is a membrane-based cell harvester. In some embodiments, cell harvesting is via a cell processing system, such as the LOVO system (manufactured by Fresenius Kabi). The term “LOVO cell processing system” also refers to any instrument or device manufactured by any vendor that can pump a solution comprising cells through a membrane or filter such as a spinning membrane or spinning filter in a sterile and/or closed system environment, allowing for continuous flow and cell processing to remove supernatant or cell culture media without pelletization. In some embodiments, the cell harvester and/or cell processing system can perform cell separation, washing, fluid-exchange, concentration, and/or other cell processing steps in a closed, sterile system.

In some embodiments, the harvest, for example, Step E according to FIG. 1, is performed from a closed system bioreactor. In some embodiments, a closed system is employed for the TIL expansion, as described herein. In some embodiments, a single bioreactor is employed. In some embodiments, the single bioreactor employed is for example a G-REX-10 or a G-REX-100. In some embodiments, the closed system bioreactor is a single bioreactor.

In some embodiments, Step E according to FIG. 1, is performed according to the processes described herein. In some embodiments, the closed system is accessed via syringes under sterile conditions in order to maintain the sterility and closed nature of the system. In some embodiments, a closed system as described in the Examples is employed.

In some embodiments, TILs are harvested according to the methods described in the Examples. In some embodiments, TILs between days 1 and 11 are harvested using the methods as described in the steps referred herein, such as in the day 11 TIL harvest in the Examples. In some embodiments, TILs between days 12 and 24 are harvested using the methods as described in the steps referred herein, such as in the Day 22 TIL harvest in the Examples. In some embodiments, TILs between days 12 and 22 are harvested using the methods as described in the steps referred herein, such as in the Day 22 TIL harvest in the Examples.

### F. STEP F: Final Formulation and Transfer to Infusion Container

After Steps A through E as provided in an exemplary order in FIG. 1 and as outlined in detail above and herein are complete, cells are transferred to a container for use in administration to a patient, such as an infusion bag or sterile vial. In some embodiments, once a therapeutically sufficient number of TILs are obtained using the expansion methods described above, they are transferred to a container for use in administration to a patient.

In some embodiments, TILs expanded using APCs of the present disclosure are administered to a patient as a pharmaceutical composition. In some embodiments, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

### VII. Gen 3 TIL Manufacturing Processes

Without being limited to any particular theory, it is believed that the priming first expansion that primes an activation of T cells followed by the rapid second expansion that boosts the activation of T cells as described in the methods of the invention allows the preparation of expanded T cells that retain a “younger” phenotype, and as such the expanded T cells of the invention are expected to exhibit greater cytotoxicity against cancer cells than T cells expanded by other methods. In particular, it is believed that an activation of T cells that is primed by exposure to an anti-CD3 antibody (e.g. OKT-3), IL-2 and optionally antigen-presenting cells (APCs) and then boosted by subsequent exposure to additional anti-CD-3 antibody (e.g. OKT-3), IL-2 and APCs as taught by the methods of the invention limits or avoids the maturation of T cells in culture, yielding a population of T cells with a less mature phenotype, which T cells are less exhausted by expansion in culture and exhibit greater cytotoxicity against cancer cells. In some embodiments, the step of rapid second expansion is split into a plurality of steps to achieve a scaling up of the culture by: (a) performing the rapid second expansion by culturing T cells in a small scale culture in a first container, e.g., a G-REX-100 MCS container, for a period of about 3 to 4 days, and then (b) effecting the transfer of the T cells in the small scale culture to a second container larger than the first container, e.g., a G-REX-500 MCS container, and culturing the T cells from the small scale culture in a larger scale culture in the second container for a period of about 4 to 7 days. In some embodiments, the step of rapid expansion is split into a plurality of steps to achieve a scaling out of the culture by: (a) performing the rapid second expansion by culturing T cells in a first small scale culture in a first container, e.g., a G-REX-100 MCS container, for a period of about 3 to 4 days, and then (b) effecting the transfer and apportioning of the T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the T cells from first small scale culture transferred to such second container is cultured in a second small scale culture for a period of about 4 to 7 days. In some embodiments, the step of rapid expansion is split into a plurality of steps to achieve a scaling out and scaling up of the culture by: (a) performing the rapid second expansion by culturing T cells in a small scale culture in a first container, e.g., a G-REX-100 MCS container, for a period of about 3 to 4 days, and then (b) effecting the transfer and apportioning of the T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX-500MCS containers, wherein in each second container the portion of the T cells from the small scale culture transferred to such second container is cultured in a larger scale culture for a period of about 4 to 7 days. In some embodiments, the step of rapid expansion is split into a plurality of steps to achieve a scaling out and scaling up of the culture by: (a) performing the rapid second expansion by culturing T cells in a small scale culture in a first container, e.g., a G-REX-100 MCS container, for a period of about 4 days, and then (b) effecting the transfer and apportioning of the T cells from the small scale culture into and amongst 2, 3 or 4 second containers that are larger in size than the first container, e.g., G-REX-500 MCS containers, wherein in each second container the portion of the T cells from the small scale culture transferred to such second container is cultured in a larger scale culture for a period of about 5 days.

In some embodiments, upon the splitting of the rapid expansion, each second container comprises at least 10⁸ TILs. In some embodiments, upon the splitting of the rapid expansion, each second container comprises at least 10⁸ TILs, at least 109 TILs, or at least 10¹⁰ TILs. In one exemplary embodiment, each second container comprises at least 10¹⁰ TILs.

In some embodiments, the first small scale TIL culture is apportioned into a plurality of subpopulations. In some embodiments, the first small scale TIL culture is apportioned into a plurality of about 2 to 5 subpopulations. In some embodiments, the first small scale TIL culture is apportioned into a plurality of about 2, 3, 4, or 5 subpopulations.

In some embodiments, after the completion of the rapid expansion, the plurality of subpopulations comprises a therapeutically effective amount of TILs. In some embodiments, after the completion of the rapid expansion, one or more subpopulations of TILs are pooled together to produce a therapeutically effective amount of TILs. In some embodiments, after the completion of the rapid expansion, each subpopulation of TILs comprises a therapeutically effective amount of TILs.

In some embodiments, the rapid expansion is performed for a period of about 1 to 5 days before being split into a plurality of steps. In some embodiments, the splitting of the rapid expansion occurs at about day 1, day 2, day 3, day 4, or day 5 after the initiation of the rapid expansion.

In some embodiments, the splitting of the rapid expansion occurs at about day 8, day 9, day 10, day 11, day 12, or day 13 after the initiation of the first expansion (i.e., pre-REP expansion). In one exemplary embodiment, the splitting of the rapid expansion occurs at about day 10 after the initiation of the priming first expansion. In another exemplary embodiment, the splitting of the rapid expansion occurs at about day 11 after the initiation of the priming first expansion.

In some embodiments, the rapid expansion is further performed for a period of about 4 to 11 days after the splitting. In some embodiments, the rapid expansion is further performed for a period of about 3 days, 4 days, 5 days, 6 days, 7 days, 8 days, 9 days, 10 days, or 11 days after the splitting.

In some embodiments, the cell culture medium used for the rapid expansion before the splitting comprises the same components as the cell culture medium used for the rapid expansion after the splitting. In some embodiments, the cell culture medium used for the rapid expansion before the splitting comprises different components from the cell culture medium used for the rapid expansion after the splitting.

In some embodiments, the cell culture medium used for the rapid expansion before the splitting comprises IL-2, optionally OKT-3 and further optionally APCs. In some embodiments, the cell culture medium used for the rapid expansion before the splitting comprises IL-2, OKT-3, and further optionally APCs. In some embodiments, the cell culture medium used for the rapid expansion before the splitting comprises IL-2, OKT-3 and APCs.

In some embodiments, the cell culture medium used for the rapid expansion before the splitting is generated by supplementing the cell culture medium in the first expansion with fresh culture medium comprising IL-2, optionally OKT-3 and further optionally APCs. In some embodiments, the cell culture medium used for the rapid expansion before the splitting is generated by supplementing the cell culture medium in the first expansion with fresh culture medium comprising IL-2, OKT-3 and APCs. In some embodiments, the cell culture medium used for the rapid expansion before the splitting is generated by replacing the cell culture medium in the first expansion with fresh cell culture medium comprising IL-2, optionally OKT-3 and further optionally APCs. In some embodiments, the cell culture medium used for the rapid expansion before the splitting is generated by replacing the cell culture medium in the first expansion with fresh cell culture medium comprising IL-2, OKT-3 and APCs.

In some embodiments, the cell culture medium used for the rapid expansion after the splitting comprises IL-2, and optionally OKT-3. In some embodiments, the cell culture medium used for the rapid expansion after the splitting comprises IL-2, and OKT-3. In some embodiments, the cell culture medium used for the rapid expansion after the splitting is generated by replacing the cell culture medium used for the rapid expansion before the splitting with fresh culture medium comprising IL-2 and optionally OKT-3. In some embodiments, the cell culture medium used for the rapid expansion after the splitting is generated by replacing the cell culture medium used for the rapid expansion before the splitting with fresh culture medium comprising IL-2 and OKT-3.

In some embodiments, the splitting of the rapid expansion occurs in a closed system.

In some embodiments, the scaling up of the TIL culture during the rapid expansion comprises adding fresh cell culture medium to the TIL culture (also referred to as feeding the TILs). In some embodiments, the feeding comprises adding fresh cell culture medium to the TIL culture frequently. In some embodiments, the feeding comprises adding fresh cell culture medium to the TIL culture at a regular interval. In some embodiments, the fresh cell culture medium is supplied to the TILs via a constant flow. In some embodiments, an automated cell expansion system such as Xuri W25 is used for the rapid expansion and feeding.

In some embodiments, the rapid second expansion is performed after the activation of T cells effected by the priming first expansion begins to decrease, abate, decay or subside.

In some embodiments, the rapid second expansion is performed after the activation of T cells effected by the priming first expansion has decreased by at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100%.

In some embodiments, the rapid second expansion is performed after the activation of T cells effected by the priming first expansion has decreased by a percentage in the range of at or about 1% to 100%.

In some embodiments, the rapid second expansion is performed after the activation of T cells effected by the priming first expansion has decreased by a percentage in the range of at or about 1% to 10%, 10% to 20%, 20% to 30%, 30% to 40%, 40% to 50%, 50% to 60%, 60% to 70%, 70% to 80%, 80% to 90%, or 90% to 100%.

In some embodiments, the rapid second expansion is performed after the activation of T cells effected by the priming first expansion has decreased by at least at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, or 99%.

In some embodiments, the rapid second expansion is performed after the activation of T cells effected by the priming first expansion has decreased by up to at or about 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100%.

In some embodiments, the decrease in the activation of T cells effected by the priming first expansion is determined by a reduction in the amount of interferon gamma released by the T cells in response to stimulation with antigen.

In some embodiments, the priming first expansion of T cells is performed during a period of up to at or about 7 days or about 8 days.

In some embodiments, the priming first expansion of T cells is performed during a period of up to at or about 1 day, 2 days, 3 days, 4 days, 5 days, 6 days, 7 days, or 8 days.

In some embodiments, the priming first expansion of T cells is performed during a period of 1 day, 2 days, 3 days, 4 days, 5 days, 6 days, 7 days, or 8 days.

In some embodiments, the rapid second expansion of T cells is performed during a period of up to at or about 11 days.

In some embodiments, the rapid second expansion of T cells is performed during a period of up to at or about 1 day, 2 days, 3 days, 4 days, 5 days, 6 days, 7 days, 8 days, 9 days, 10 days or 11 days.

In some embodiments, the rapid second expansion of T cells is performed during a period of 1 day, 2 days, 3 days, 4 days, 5 days, 6 days, 7 days, 8 days, 9 days, 10 days or 11 days.

In some embodiments, the priming first expansion of T cells is performed during a period of from at or about 1 day to at or about 7 days and the rapid second expansion of T cells is performed during a period of from at or about 1 day to at or about 11 days.

In some embodiments, the priming first expansion of T cells is performed during a period of up to at or about 1 day, 2 days, 3 days, 4 days, 5 days, 6 days, 7 days, or 8 days and the rapid second expansion of T cells is performed during a period of up to at or about 1 day, 2 days, 3 days, 4 days, 5 days, 6 days, 7 days, 8 days, 9 days, 10 days or 11 days.

In some embodiments, the priming first expansion of T cells is performed during a period of from at or about 1 day to at or about 8 days and the rapid second expansion of T cells is performed during a period of from at or about 1 day to at or about 9 days.

In some embodiments, the priming first expansion of T cells is performed during a period of 8 days and the rapid second expansion of T cells is performed during a period of 9 days.

In some embodiments, the priming first expansion of T cells is performed during a period of from at or about 1 day to at or about 7 days and the rapid second expansion of T cells is performed during a period of from at or about 1 day to at or about 9 days.

In some embodiments, the priming first expansion of T cells is performed during a period of 7 days and the rapid second expansion of T cells is performed during a period of 9 days.

In some embodiments, the T cells are tumor infiltrating lymphocytes (TILs).

In some embodiments, the T cells are marrow infiltrating lymphocytes (MILs).

In some embodiments, the T cells are peripheral blood lymphocytes (PBLs).

In some embodiments, the T cells are obtained from a donor suffering from a cancer.

In some embodiments, the T cells are TILs obtained from a tumor excised from a patient suffering from a cancer.

In some embodiments, the T cells are MILs obtained from bone marrow of a patient suffering from a hematologic malignancy.

In some embodiments, the T cells are PBLs obtained from peripheral blood mononuclear cells (PBMCs) from a donor. In some embodiments, the donor is suffering from a cancer. In some embodiments, the cancer is the cancer is selected from the group consisting of melanoma, ovarian cancer, endometrial cancer, thyroid cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the donor is suffering from a tumor. In some embodiments, the tumor is a liquid tumor. In some embodiments, the tumor is a solid tumor. In some embodiments, the donor is suffering from a hematologic malignancy.

In certain aspects of the present disclosure, immune effector cells, e.g., T cells, can be obtained from a unit of blood collected from a subject using any number of techniques known to the skilled artisan, such as FICOLL separation. In one preferred aspect, cells from the circulating blood of an individual are obtained by apheresis. The apheresis product typically contains lymphocytes, including T cells, monocytes, granulocytes, B cells, other nucleated white blood cells, red blood cells, and platelets. In one aspect, the cells collected by apheresis may be washed to remove the plasma fraction and, optionally, to place the cells in an appropriate buffer or media for subsequent processing steps. In some embodiments, the cells are washed with phosphate buffered saline (PBS). In an alternative embodiment, the wash solution lacks calcium and may lack magnesium or may lack many if not all divalent cations. In one aspect, T cells are isolated from peripheral blood lymphocytes by lysing the red blood cells and depleting the monocytes, for example, by centrifugation through a PERCOLL gradient or by counterflow centrifugal elutriation.

In some embodiments, the T cells are PBLs separated from whole blood or apheresis product enriched for lymphocytes from a donor. In some embodiments, the donor is suffering from a cancer. In some embodiments, the cancer is the cancer is selected from the group consisting of melanoma, ovarian cancer, endometrial cancer, thyroid cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the cancer is selected from the group consisting of melanoma, ovarian cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, and renal cell carcinoma. In some embodiments, the donor is suffering from a tumor. In some embodiments, the tumor is a liquid tumor. In some embodiments, the tumor is a solid tumor.

In some embodiments, the donor is suffering from a hematologic malignancy. In some embodiments, the PBLs are isolated from whole blood or apheresis product enriched for lymphocytes by using positive or negative selection methods, i.e., removing the PBLs using a marker(s), e.g., CD3+ CD45+, for T cell phenotype, or removing non-T cell phenotype cells, leaving PBLs. In other embodiments, the PBLs are isolated by gradient centrifugation. Upon isolation of PBLs from donor tissue, the priming first expansion of PBLs can be initiated by seeding a suitable number of isolated PBLs (in some embodiments, approximately 1×10⁷ PBLs) in the priming first expansion culture according to the priming first expansion step of any of the methods described herein.

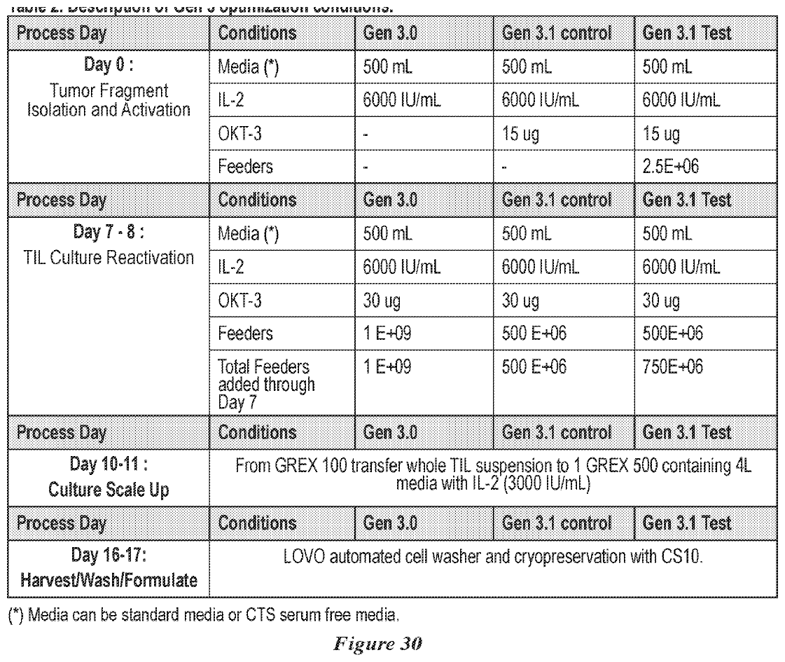

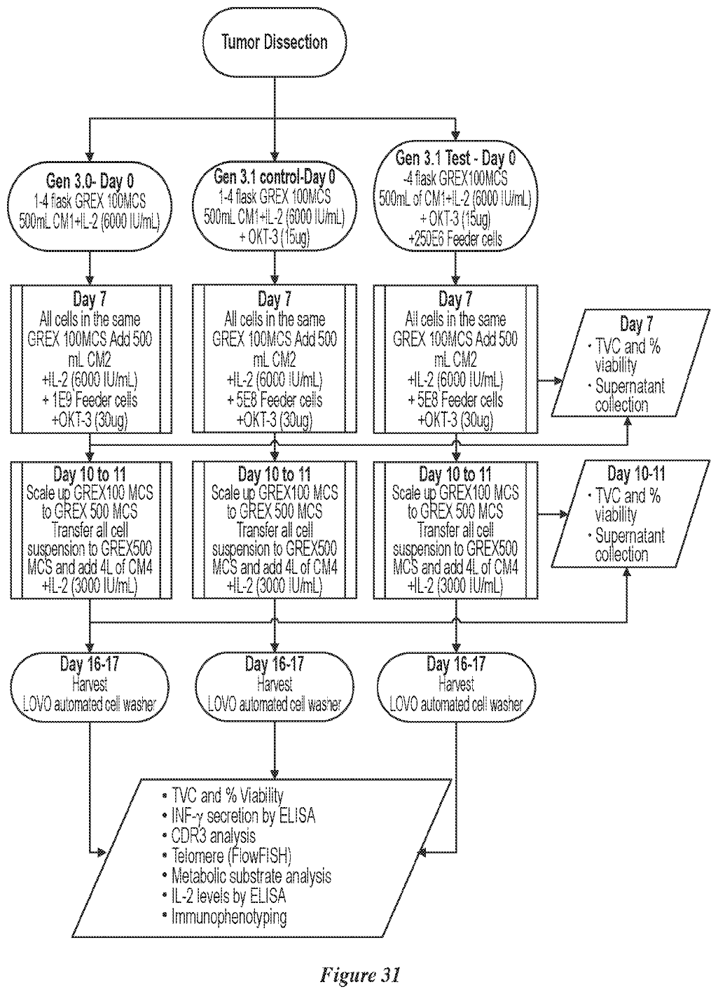

An exemplary TIL process known as process 3 (also referred to herein as Gen 3) containing some of these features is depicted in FIG. 8 (in particular, e.g., FIG. 8B and/or FIG. 8C and/or FIG. 8D), and some of the advantages of this embodiment of the present invention over Gen 2 are described in FIGS. 1, 2, 8, 30, and 31 (in particular, e.g., FIG. 8A and/or FIG. 8B and/or FIG. 8C and/or FIG. 8D). Embodiments of Gen 3 are shown in FIGS. 1, 8, and 30 (in particular, e.g., FIG. 8A and/or FIG. 8B and/or FIG. 8C and/or FIG. 8D). Process 2A or Gen 2 or Gen 2A is also described in U.S. Patent Publication No. 2018/0280436, incorporated by reference herein in its entirety. The Gen 3 process is also described in International Patent Publication WO 2020/096988.

As discussed and generally outlined herein, TILs are taken from a patient sample and manipulated to expand their number prior to transplant into a patient using the TIL expansion process described herein and referred to as Gen 3. In some embodiments, the TILs may be optionally genetically manipulated as discussed below. In some embodiments, the TILs may be cryopreserved prior to or after expansion. Once thawed, they may also be restimulated to increase their metabolism prior to infusion into a patient.

In some embodiments, the priming first expansion (including processes referred herein as the pre-Rapid Expansion (Pre-REP), as well as processes shown in FIG. 8 (in particular, e.g., FIG. 8A and/or FIG. 8B and/or FIG. 8C and/or FIG. 8D) as Step B) is shortened to 1 to 8 days and the rapid second expansion (including processes referred to herein as Rapid Expansion Protocol (REP) as well as processes shown in FIG. 8 (in particular, e.g., FIG. 8A and/or FIG. 8B and/or FIG. 8C and/or FIG. 8D) as Step D) is shortened to 1 to 9 days, as discussed in detail below as well as in the examples and figures. In some embodiments, the priming first expansion (including processes referred herein as the pre-Rapid Expansion (Pre-REP), as well as processes shown in FIG. 8 (in particular, e.g., FIG. 8A and/or FIG. 8B and/or FIG. 8C and/or FIG. 8D) as Step B) is shortened to 1 to 8 days and the rapid second expansion (including processes referred to herein as Rapid Expansion Protocol (REP) as well as processes shown in FIG. 8 (in particular, e.g., FIG. 8A and/or FIG. 8B and/or FIG. 8C and/or FIG. 8D) as Step D) is shortened to 1 to 8 days, as discussed in detail below as well as in the examples and figures. In some embodiments, the priming first expansion (including processes referred herein as the pre-Rapid Expansion (Pre-REP), as well as processes shown in FIG. 8 (in particular, e.g., FIG. 8A and/or FIG. 8B and/or FIG. 8C and/or FIG. 8D) as Step B) is shortened to 1 to 7 days and the rapid second expansion (including processes referred to herein as Rapid Expansion Protocol (REP) as well as processes shown in FIG. 8 (in particular, e.g., FIG. 8A and/or FIG. 8B and/or FIG. 8C and/or FIG. 8D) as Step D) is shortened to 1 to 9 days, as discussed in detail below as well as in the examples and figures. In some embodiments, the priming first expansion (including processes referred herein as the pre-Rapid Expansion (Pre-REP), as well as processes shown in FIG. 8 (in particular, e.g., FIG. 1B and/or FIG. 8C) as Step B) is 1 to 7 days and the rapid second expansion (including processes referred to herein as Rapid Expansion Protocol (REP) as well as processes shown in FIG. 8 (in particular, e.g., FIG. 8A and/or FIG. 8B and/or FIG. 8C and/or FIG. 8D) as Step D) is 1 to 10 days, as discussed in detail below as well as in the examples and figures. In some embodiments, the priming first expansion (for example, an expansion described as Step B in FIG. 8 (in particular, e.g., FIG. 8A and/or FIG. 8B and/or FIG. 8C and/or FIG. 8D) is shortened to 8 days and the rapid second expansion (for example, an expansion as described in Step D in FIG. 8 (in particular, e.g., FIG. 8A and/or FIG. 8B and/or FIG. 8C and/or FIG. 8D)) is 7 to 9 days. In some embodiments, the priming first expansion (for example, an expansion described as Step B in FIG. 8 (in particular, e.g., FIG. 8A and/or FIG. 8B and/or FIG. 8C and/or FIG. 8D)) is 8 days and the rapid second expansion (for example, an expansion as described in Step D in FIG. 8 (in particular, e.g., FIG. 8A and/or FIG. 8B and/or FIG. 8C and/or FIG. 8D)) is 8 to 9 days. In some embodiments, the priming first expansion (for example, an expansion described as Step B in FIG. 8 (in particular, e.g., FIG. 8A and/or FIG. 8B and/or FIG. 8C and/or FIG. 8D)) is shortened to 7 days and the rapid second expansion (for example, an expansion as described in Step D in FIG. 8 (in particular, e.g., FIG. 8A and/or FIG. 8B and/or FIG. 8C and/or FIG. 8D)) is 7 to 8 days. In some embodiments, the priming first expansion (for example, an expansion described as Step B in FIG. 8 (in particular, e.g., FIG. 8A and/or FIG. 8B and/or FIG. 8C and/or FIG. 8D)) is shortened to 8 days and the rapid second expansion (for example, an expansion as described in Step D in FIG. 8 (in particular, e.g., FIG. 8A and/or FIG. 8B and/or FIG. 8C and/or FIG. 8D)) is 8 days. In some embodiments, the priming first expansion (for example, an expansion described as Step B in FIG. 8 (in particular, e.g., FIG. 8A and/or FIG. 8B and/or FIG. 8C and/or FIG. 8D)) is 8 days and the rapid second expansion (for example, an expansion as described in Step D in FIG. 8 (in particular, e.g., FIG. 8A and/or FIG. 8B and/or FIG. 8C and/or FIG. 8D)) is 9 days. In some embodiments, the priming first expansion (for example, an expansion described as Step B in FIG. 8 (in particular, e.g., FIG. 8A and/or FIG. 8B and/or FIG. 8C and/or FIG. 8D)) is 8 days and the rapid second expansion (for example, an expansion as described in Step D in FIG. 8 (in particular, e.g., FIG. 8A and/or FIG. 8B and/or FIG. 8C and/or FIG. 8D)) is 10 days. In some embodiments, the priming first expansion (for example, an expansion described as Step B in FIG. 8 (in particular, e.g., FIG. 8A and/or FIG. 8B and/or FIG. 8C and/or FIG. 8D)) is 7 days and the rapid second expansion (for example, an expansion as described in Step D in FIG. 8 (in particular, e.g., FIG. 8A and/or FIG. 8B and/or FIG. 8C and/or FIG. 8D)) is 7 to 10 days. In some embodiments, the priming first expansion (for example, an expansion described as Step B in FIG. 8 (in particular, e.g., FIG. 8A and/or FIG. 8B and/or FIG. 8C and/or FIG. 8D)) is 7 days and the rapid second expansion (for example, an expansion as described in Step D in FIG. 8 (in particular, e.g., FIG. 8A and/or FIG. 8B and/or FIG. 8C and/or FIG. 8D)) is 8 to 10 days. In some embodiments, the priming first expansion (for example, an expansion described as Step B in FIG. 8 (in particular, e.g., FIG. 8A and/or FIG. 8B and/or FIG. 8C and/or FIG. 8D)) is 7 days and the rapid second expansion (for example, an expansion as described in Step D in FIG. 8 (in particular, e.g., FIG. 8A and/or FIG. 8B and/or FIG. 8C and/or FIG. 8D)) is 9 to 10 days. In some embodiments, the priming first expansion (for example, an expansion described as Step B in FIG. 8 (in particular, e.g., FIG. 8A and/or FIG. 8B and/or FIG. 8C and/or FIG. 8D) is shortened to 7 days and the rapid second expansion (for example, an expansion as described in Step D in FIG. 8 (in particular, e.g., FIG. 8A and/or FIG. 8B and/or FIG. 8C and/or FIG. 8D) is 7 to 9 days. In some embodiments, the combination of the priming first expansion and rapid second expansion (for example, expansions described as Step B and Step D in FIG. 8 (in particular, e.g., FIG. 1B and/or FIG. 8C) is 14-16 days, as discussed in detail below and in the examples and figures. Particularly, it is considered that certain embodiments of the present invention comprise a priming first expansion step in which TILs are activated by exposure to an anti-CD3 antibody, e.g., OKT-3 in the presence of IL-2 or exposure to an antigen in the presence of at least IL-2 and an anti-CD3 antibody e.g. OKT-3. In certain embodiments, the TILs which are activated in the priming first expansion step as described above are a first population of TILs i.e., which are a primary cell population.

The “Step” Designations A, B, C, etc., below are in reference to the non-limiting example in FIG. 8 (in particular, e.g., FIG. 8A and/or FIG. 8B and/or FIG. 8C and/or FIG. 8D) and in reference to certain non-limiting embodiments described herein. The ordering of the Steps below and in FIG. 8 (in particular, e.g., FIG. 8A and/or FIG. 8B and/or FIG. 8C and/or FIG. 8D) is exemplary and any combination or order of steps, as well as additional steps, repetition of steps, and/or omission of steps is contemplated by the present application and the methods disclosed herein.

### A. STEP A: Obtain Patient Tumor Sample

In general, TILs are initially obtained from a patient tumor sample (“primary TILs”) or from circulating lymphocytes, such as peripheral blood lymphocytes, including peripheral blood lymphocytes having TIL-like characteristics, and are then expanded into a larger population for further manipulation as described herein, optionally cryopreserved, and optionally evaluated for phenotype and metabolic parameters as an indication of TIL health.

A patient tumor sample may be obtained using methods known in the art, generally via surgical resection, needle biopsy or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. The solid tumor may be of any cancer type, including, but not limited to, breast, pancreatic, prostate, colorectal, lung, brain, renal, stomach, and skin (including but not limited to squamous cell carcinoma, basal cell carcinoma, and melanoma). In some embodiments, the cancer is selected from cervical cancer, head and neck cancer (including, for example, head and neck squamous cell carcinoma (HNSCC)), glioblastoma (GBM), gastrointestinal cancer, ovarian cancer, sarcoma, pancreatic cancer, bladder cancer, breast cancer, triple negative breast cancer, and non-small cell lung carcinoma. In some embodiments, the cancer is melanoma. In some embodiments, useful TILs are obtained from malignant melanoma tumors, as these have been reported to have particularly high levels of TILs.

Once obtained, the tumor sample is generally fragmented using sharp dissection into small pieces of between 1 to about 8 mm³, with from about 2-3 mm³ being particularly useful. The TILs are cultured from these fragments using enzymatic tumor digests. Such tumor digests may be produced by incubation in enzymatic media (e.g., Roswell Park Memorial Institute (RPMI) 1640 buffer, 2 mM glutamate, 10 mcg/mL gentamicine, 30 units/mL of DNase and 1.0 mg/mL of collagenase) followed by mechanical dissociation (e.g., using a tissue dissociator). Tumor digests may be produced by placing the tumor in enzymatic media and mechanically dissociating the tumor for approximately 1 minute, followed by incubation for 30 minutes at 37° C. in 5% CO₂, followed by repeated cycles of mechanical dissociation and incubation under the foregoing conditions until only small tissue pieces are present. At the end of this process, if the cell suspension contains a large number of red blood cells or dead cells, a density gradient separation using FICOLL branched hydrophilic polysaccharide may be performed to remove these cells. Alternative methods known in the art may be used, such as those described in U.S. Patent Application Publication No. 2012/0244133 A1, the disclosure of which is incorporated by reference herein. Any of the foregoing methods may be used in any of the embodiments described herein for methods of expanding TILs or methods treating a cancer.

As indicated above, in some embodiments, the TILs are derived from solid tumors. In some embodiments, the solid tumors are not fragmented. In some embodiments, the solid tumors are not fragmented and are subjected to enzymatic digestion as whole tumors. In some embodiments, the tumors are digested in in an enzyme mixture comprising collagenase, DNase, and hyaluronidase. In some embodiments, the tumors are digested in in an enzyme mixture comprising collagenase, DNase, and hyaluronidase for 1-2 hours. In some embodiments, the tumors are digested in in an enzyme mixture comprising collagenase, DNase, and hyaluronidase for 1-2 hours at 37° C., 5% CO₂. In some embodiments, the tumors are digested in in an enzyme mixture comprising collagenase, DNase, and hyaluronidase for 1-2 hours at 37° C., 5% CO₂ with rotation. In some embodiments, the tumors are digested overnight with constant rotation. In some embodiments, the tumors are digested overnight at 37° C., 5% CO₂ with constant rotation. In some embodiments, the whole tumor is combined with the enzymes to form a tumor digest reaction mixture.

In some embodiments, the tumor is reconstituted with the lyophilized enzymes in a sterile buffer. In some embodiments, the buffer is sterile HBSS.

In some embodiments, the enzyme mixture comprises collagenase. In some embodiments, the collagenase is collagenase IV. In some embodiments, the working stock for the collagenase is a 100 mg/mL 10× working stock.

In some embodiments, the enzyme mixture comprises DNAse. In some embodiments, the working stock for the DNAse is a 10,000 IU/mL 10× working stock.

In some embodiments, the enzyme mixture comprises hyaluronidase. In some embodiments, the working stock for the hyaluronidase is a 10 mg/mL 10× working stock.

In some embodiments, the enzyme mixture comprises 10 mg/mL collagenase, 1000 IU/mL DNAse, and 1 mg/mL hyaluronidase.

In some embodiments, the enzyme mixture comprises 10 mg/mL collagenase, 500 IU/mL DNAse, and 1 mg/mL hyaluronidase.

In general, the cell suspension obtained from the tumor is called a “primary cell population” or a “freshly obtained” or a “freshly isolated” cell population. In certain embodiments, the freshly obtained cell population of TILs is exposed to a cell culture medium comprising antigen presenting cells, IL-12 and OKT-3.

In some embodiments, fragmentation includes physical fragmentation, including, for example, dissection as well as digestion. In some embodiments, the fragmentation is physical fragmentation. In some embodiments, the fragmentation is dissection. In some embodiments, the fragmentation is by digestion. In some embodiments, TILs can be initially cultured from enzymatic tumor digests and tumor fragments obtained from patients. In some embodiments, TILs can be initially cultured from enzymatic tumor digests and tumor fragments obtained from patients.

In some embodiments, where the tumor is a solid tumor, the tumor undergoes physical fragmentation after the tumor sample is obtained in, for example, Step A (as provided in FIG. 8 (in particular, e.g., FIG. 8A and/or FIG. 8B and/or FIG. 8C and/or FIG. 8D)). In some embodiments, the fragmentation occurs before cryopreservation. In some embodiments, the fragmentation occurs after cryopreservation. In some embodiments, the fragmentation occurs after obtaining the tumor and in the absence of any cryopreservation. In some embodiments, the step of fragmentation is an in vitro or ex-vivo process. In some embodiments, the tumor is fragmented and 10, 20, 30, 40 or more fragments or pieces are placed in each container for the priming first expansion. In some embodiments, the tumor is fragmented and 30 or 40 fragments or pieces are placed in each container for the priming first expansion. In some embodiments, the tumor is fragmented and 40 fragments or pieces are placed in each container for the priming first expansion. In some embodiments, the multiple fragments comprise about 4 to about 50 fragments, wherein each fragment has a volume of about 27 mm³. In some embodiments, the multiple fragments comprise about 30 to about 60 fragments with a total volume of about 1300 mm³ to about 1500 mm³. In some embodiments, the multiple fragments comprise about 50 fragments with a total volume of about 1350 mm³. In some embodiments, the multiple fragments comprise about 50 fragments with a total mass of about 1 gram to about 1.5 grams. In some embodiments, the multiple fragments comprise about 4 fragments.

In some embodiments, the TILs are obtained from tumor fragments. In some embodiments, the tumor fragment is obtained by sharp dissection. In some embodiments, the tumor fragment is between about 1 mm³ and 10 mm³. In some embodiments, the tumor fragment is between about 1 mm³ and 8 mm³. In some embodiments, the tumor fragment is about 1 mm³. In some embodiments, the tumor fragment is about 2 mm³. In some embodiments, the tumor fragment is about 3 mm³. In some embodiments, the tumor fragment is about 4 mm³. In some embodiments, the tumor fragment is about 5 mm³. In some embodiments, the tumor fragment is about 6 mm³. In some embodiments, the tumor fragment is about 7 mm³. In some embodiments, the tumor fragment is about 8 mm³. In some embodiments, the tumor fragment is about 9 mm³. In some embodiments, the tumor fragment is about 10 mm³. In some embodiments, the tumor fragments are 1-4 mmx 1-4 mm×1-4 mm. In some embodiments, the tumor fragments are 1 mm×1 mm×1 mm. In some embodiments, the tumor fragments are 2 mmx 2 mm×2 mm. In some embodiments, the tumor fragments are 3 mm×3 mm×3 mm. In some embodiments, the tumor fragments are 4 mmx 4 mm×4 mm.

In some embodiments, the tumors are fragmented in order to minimize the amount of hemorrhagic, necrotic, and/or fatty tissues on each piece. In some embodiments, the tumors are fragmented in order to minimize the amount of hemorrhagic tissue on each piece. In some embodiments, the tumors are fragmented in order to minimize the amount of necrotic tissue on each piece. In some embodiments, the tumors are fragmented in order to minimize the amount of fatty tissue on each piece. In certain embodiments, the step of fragmentation of the tumor is an in vitro or ex-vivo method.

In some embodiments, the tumor fragmentation is performed in order to maintain the tumor internal structure. In some embodiments, the tumor fragmentation is performed without preforming a sawing motion with a scalpel. In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, the cell suspension prior to the priming first expansion step is called a “primary cell population” or a “freshly obtained” or “freshly isolated” cell population.

In some embodiments, cells can be optionally frozen after sample isolation (e.g., after obtaining the tumor sample and/or after obtaining the cell suspension from the tumor sample) and stored frozen prior to entry into the expansion described in Step B, which is described in further detail below, as well as exemplified in FIG. 8 (in particular, e.g., FIG. 8B).

### 1. Core/Small Biopsy Derived TILs

In some embodiments, TILs are initially obtained from a patient tumor sample (“primary TILs”) obtained by a core biopsy or similar procedure and then expanded into a larger population for further manipulation as described herein, optionally cryopreserved, and optionally evaluated for phenotype and metabolic parameters.

In some embodiments, a patient tumor sample may be obtained using methods known in the art, generally via small biopsy, core biopsy, needle biopsy or other means for obtaining a sample that contains a mixture of tumor and TIL cells. In general, the tumor sample may be from any solid tumor, including primary tumors, invasive tumors or metastatic tumors. The tumor sample may also be a liquid tumor, such as a tumor obtained from a hematological malignancy. In some embodiments, the sample can be from multiple small tumor samples or biopsies. In some embodiments, the sample can comprise multiple tumor samples from a single tumor from the same patient. In some embodiments, the sample can comprise multiple tumor samples from one, two, three, or four tumors from the same patient. In some embodiments, the sample can comprise multiple tumor samples from multiple tumors from the same patient. The solid tumor may be a lung and/or non-small cell lung carcinoma (NSCLC).

In general, the cell suspension obtained from the tumor core or fragment is called a “primary cell population” or a “freshly obtained” or a “freshly isolated” cell population. In certain embodiments, the freshly obtained cell population of TILs is exposed to a cell culture medium comprising antigen presenting cells, IL-2 and OKT-3.

In some embodiments, if the tumor is metastatic and the primary lesion has been efficiently treated/removed in the past, removal of one of the metastatic lesions may be needed. In some embodiments, the least invasive approach is to remove a skin lesion, or a lymph node on the neck or axillary area when available. In some embodiments, a skin lesion is removed or small biopsy thereof is removed. In some embodiments, a lymph node or small biopsy thereof is removed. In some embodiments, the tumor is a melanoma. In some embodiments, the small biopsy for a melanoma comprises a mole or portion thereof.

In some embodiments, the small biopsy is a punch biopsy. In some embodiments, the punch biopsy is obtained with a circular blade pressed into the skin. In some embodiments, the punch biopsy is obtained with a circular blade pressed into the skin. around a suspicious mole. In some embodiments, the punch biopsy is obtained with a circular blade pressed into the skin, and a round piece of skin is removed. In some embodiments, the small biopsy is a punch biopsy and round portion of the tumor is removed.

In some embodiments, the small biopsy is an excisional biopsy. In some embodiments, the small biopsy is an excisional biopsy and the entire mole or growth is removed. In some embodiments, the small biopsy is an excisional biopsy and the entire mole or growth is removed along with a small border of normal-appearing skin.

In some embodiments, the small biopsy is an incisional biopsy. In some embodiments, the small biopsy is an incisional biopsy and only the most irregular part of a mole or growth is taken. In some embodiments, the small biopsy is an incisional biopsy and the incisional biopsy is used when other techniques can't be completed, such as if a suspicious mole is very large.

In some embodiments, the small biopsy is a lung biopsy. In some embodiments, the small biopsy is obtained by bronchoscopy. Generally, bronchoscopy, the patient is put under anesthesia, and a small tool goes through the nose or mouth, down the throat, and into the bronchial passages, where small tools are used to remove some tissue. In some embodiments, where the tumor or growth cannot be reached via bronchoscopy, a transthoracic needle biopsy can be employed. Generally, for a transthoracic needle biopsy, the patient is also under anesthesia and a needle is inserted through the skin directly into the suspicious spot to remove a small sample of tissue. In some embodiments, a transthoracic needle biopsy may require interventional radiology (for example, the use of x-rays or CT scan to guide the needle). In some embodiments, the small biopsy is obtained by needle biopsy. In some embodiments, the small biopsy is obtained endoscopic ultrasound (for example, an endoscope with a light and is placed through the mouth into the esophagus). In some embodiments, the small biopsy is obtained surgically.

In some embodiments, the small biopsy is ahead and neck biopsy. In some embodiments, the small biopsy is an incisional biopsy. In some embodiments, the small biopsy is an incisional biopsy, wherein a small piece of tissue is cut from an abnormal-looking area. In some embodiments, if the abnormal region is easily accessed, the sample may be taken without hospitalization. In some embodiments, if the tumor is deeper inside the mouth or throat, the biopsy may need to be done in an operating room, with general anesthesia. In some embodiments, the small biopsy is an excisional biopsy. In some embodiments, the small biopsy is an excisional biopsy, wherein the whole area is removed. In some embodiments, the small biopsy is a fine needle aspiration (FNA). In some embodiments, the small biopsy is a fine needle aspiration (FNA), wherein a very thin needle attached to a syringe is used to extract (aspirate) cells from a tumor or lump. In some embodiments, the small biopsy is a punch biopsy. In some embodiments, the small biopsy is a punch biopsy, wherein punch forceps are used to remove a piece of the suspicious area.

In some embodiments, the small biopsy is a cervical biopsy. In some embodiments, the small biopsy is obtained via colposcopy. Generally, colposcopy methods employ the use of a lighted magnifying instrument attached to magnifying binoculars (a colposcope) which is then used to biopsy a small section of the surface of the cervix. In some embodiments, the small biopsy is a conization/cone biopsy. In some embodiments, the small biopsy is a conization/cone biopsy, wherein an outpatient surgery may be needed to remove a larger piece of tissue from the cervix. In some embodiments, the cone biopsy, in addition to helping to confirm a diagnosis, a cone biopsy can serve as an initial treatment.

The term “solid tumor” refers to an abnormal mass of tissue that usually does not contain cysts or liquid areas. Solid tumors may be benign or malignant. The term “solid tumor cancer refers to malignant, neoplastic, or cancerous solid tumors. Solid tumor cancers include cancers of the lung. In some embodiments, the cancer is melanoma. In some embodiments, the cancer is non-small cell lung carcinoma (NSCLC). The tissue structure of solid tumors includes interdependent tissue compartments including the parenchyma (cancer cells) and the supporting stromal cells in which the cancer cells are dispersed and which may provide a supporting microenvironment.

In some embodiments, the sample from the tumor is obtained as a fine needle aspirate (FNA), a core biopsy, a small biopsy (including, for example, a punch biopsy). In some embodiments, sample is placed first into a G-REX-10. In some embodiments, sample is placed first into a G-REX-10 when there are 1 or 2 core biopsy and/or small biopsy samples. In some embodiments, sample is placed first into a G-REX-100 when there are 3, 4, 5, 6, 8, 9, or 10 or more core biopsy and/or small biopsy samples. In some embodiments, sample is placed first into a G-REX-500 when there are 3, 4, 5, 6, 8, 9, or 10 or more core biopsy and/or small biopsy samples.

The FNA can be obtained from a skin tumor, including, for example, a melanoma. In some embodiments, the FNA is obtained from a skin tumor, such as a skin tumor from a patient with metastatic melanoma. In some cases, the patient with melanoma has previously undergone a surgical treatment.

The FNA can be obtained from a lung tumor, including, for example, an NSCLC. In some embodiments, the FNA is obtained from a lung tumor, such as a lung tumor from a patient with non-small cell lung cancer (NSCLC). In some cases, the patient with NSCLC has previously undergone a surgical treatment.

TILs described herein can be obtained from an FNA sample. In some cases, the FNA sample is obtained or isolated from the patient using a fine gauge needle ranging from an 18 gauge needle to a 25 gauge needle. The fine gauge needle can be 18 gauge, 19 gauge, 20 gauge, 21 gauge, 22 gauge, 23 gauge, 24 gauge, or 25 gauge. In some embodiments, the FNA sample from the patient can contain at least 400,000 TILs, e.g., 400,000 TILs, 450,000 TILs, 500,000 TILs, 550,000 TILs, 600,000 TILs, 650,000 TILs, 700,000 TILs, 750,000 TILs, 800,000 TILs, 850,000 TILs, 900,000 TILs, 950,000 TILs, or more.

In some cases, the TILs described herein are obtained from a core biopsy sample. In some cases, the core biopsy sample is obtained or isolated from the patient using a surgical or medical needle ranging from an 11 gauge needle to a 16 gauge needle. The needle can be 11 gauge, 12 gauge, 13 gauge, 14 gauge, 15 gauge, or 16 gauge. In some embodiments, the core biopsy sample from the patient can contain at least 400,000 TILs, e.g., 400,000 TILs, 450,000 TILs, 500,000 TILs, 550,000 TILs, 600,000 TILs, 650,000 TILs, 700,000 TILs, 750,000 TILs, 800,000 TILs, 850,000 TILs, 900,000 TILs, 950,000 TILs, or more.

In general, the harvested cell suspension is called a “primary cell population” or a “freshly harvested” cell population.

In some embodiments, the TILs are not obtained from tumor digests. In some embodiments, the solid tumor cores are not fragmented.

In some embodiments, the TILs are obtained from tumor digests. In some embodiments, tumor digests were generated by incubation in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). After placing the tumor in enzyme media, the tumor can be mechanically dissociated for approximately 1 minute. The solution can then be incubated for 30 minutes at 37° C. in 5% CO₂ and it then mechanically disrupted again for approximately 1 minute. After being incubated again for 30 minutes at 37° C. in 5% CO₂, the tumor can be mechanically disrupted a third time for approximately 1 minute. In some embodiments, after the third mechanical disruption if large pieces of tissue were present, 1 or 2 additional mechanical dissociations were applied to the sample, with or without 30 additional minutes of incubation at 37° C. in 5% CO₂. In some embodiments, at the end of the final incubation if the cell suspension contained a large number of red blood cells or dead cells, a density gradient separation using Ficoll can be performed to remove these cells.

In some embodiments, obtaining the first population of TILs comprises a multilesional sampling method.

Tumor dissociating enzyme mixtures can include one or more dissociating (digesting) enzymes such as, but not limited to, collagenase (including any blend or type of collagenase), Accutase™, Accumax™, hyaluronidase, neutral protease (dispase), chymotrypsin, chymopapain, trypsin, caseinase, elastase, papain, protease type XIV (pronase), deoxyribonuclease I (DNase), trypsin inhibitor, any other dissociating or proteolytic enzyme, and any combination thereof.

In some embodiments, the dissociating enzymes are reconstituted from lyophilized enzymes. In some embodiments, lyophilized enzymes are reconstituted in an amount of sterile buffer such as Hank's balance salt solution (HBSS).

In some instances, collagenase (such as animal free-type 1 collagenase) is reconstituted in 10 mL of sterile HBSS or another buffer. The lyophilized stock enzyme may be at a concentration of 2892 PZ U/vial. In some embodiments, collagenase is reconstituted in 5 mL to 15 mL buffer. In some embodiments, after reconstitution the collagenase stock ranges from about 100 PZ U/mL-about 400 PZ U/mL, e.g., about 100 PZ U/mL-about 400 PZ U/mL, about 100 PZ U/mL-about 350 PZ U/mL, about 100 PZ U/mL-about 300 PZ U/mL, about 150 PZ U/mL-about 400 PZ U/mL, about 100 PZ U/mL, about 150 PZ U/mL, about 200 PZ U/mL, about 210 PZ U/mL, about 220 PZ U/mL, about 230 PZ U/mL, about 240 PZ U/mL, about 250 PZ U/mL, about 260 PZ U/mL, about 270 PZ U/mL, about 280 PZ U/mL, about 289.2 PZ U/mL, about 300 PZ U/mL, about 350 PZ U/mL, or about 400 PZ U/mL.

In some embodiments neutral protease is reconstituted in 1 mL of sterile HBSS or another buffer. The lyophilized stock enzyme may be at a concentration of 175 DMC U/vial. In some embodiments, after reconstitution the neutral protease stock ranges from about 100 DMC/mL-about 400 DMC/mL, e.g., about 100 DMC/mL-about 400 DMC/mL, about 100 DMC/mL-about 350 DMC/mL, about 100 DMC/mL-about 300 DMC/mL, about 150 DMC/mL-about 400 DMC/mL, about 100 DMC/mL, about 110 DMC/mL, about 120 DMC/mL, about 130 DMC/mL, about 140 DMC/mL, about 150 DMC/mL, about 160 DMC/mL, about 170 DMC/mL, about 175 DMC/mL, about 180 DMC/mL, about 190 DMC/mL, about 200 DMC/mL, about 250 DMC/mL, about 300 DMC/mL, about 350 DMC/mL, or about 400 DMC/mL.

In some embodiments, DNAse I is reconstituted in 1 mL of sterile HBSS or another buffer. The lyophilized stock enzyme was at a concentration of 4 KU/vial. In some embodiments, after reconstitution the DNase I stock ranges from about 1 KU/mL to 10 KU/mL, e.g., about 1 KU/mL, about 2 KU/mL, about 3 KU/mL, about 4 KU/mL, about 5 KU/mL, about 6 KU/mL, about 7 KU/mL, about 8 KU/mL, about 9 KU/mL, or about 10 KU/mL.

In some embodiments, the stock of enzymes could change so verify the concentration of the lyophilized stock and amend the final amount of enzyme added to the digest cocktail accordingly

In some embodiments, the enzyme mixture includes about 10.2-ul of neutral protease (0.36 DMC U/mL), 21.3-ul of collagenase (1.2 PZ/mL) and 250-ul of DNAse 1(200 U/mL) in about 4.7 mL of sterile HBSS.

### 2. Pleural Effusion T-cells and TILs

In some embodiments, the sample is a pleural fluid sample. In some embodiments, the source of the T-cells or TILs for expansion according to the processes described herein is a pleural fluid sample. In some embodiments, the sample is a pleural effusion derived sample. In some embodiments, the source of the T-cells or TILs for expansion according to the processes described herein is a pleural effusion derived sample. See, for example, methods described in U.S. Patent Publication US 2014/0295426, incorporated herein by reference in its entirety for all purposes.

In some embodiments, any pleural fluid or pleural effusion suspected of and/or containing TILs can be employed. Such a sample may be derived from a primary or metastatic lung cancer, such as NSCLC or SCLC. In some embodiments, the sample may be secondary metastatic cancer cells which originated from another organ, e.g., breast, ovary, colon or prostate. In some embodiments, the sample for use in the expansion methods described herein is a pleural exudate. In some embodiments, the sample for use in the expansion methods described herein is a pleural transudate. Other biological samples may include other serous fluids containing TILs, including, e.g., ascites fluid from the abdomen or pancreatic cyst fluid. Ascites fluid and pleural fluids involve very similar chemical systems; both the abdomen and lung have mesothelial lines and fluid forms in the pleural space and abdominal spaces in the same matter in malignancies and such fluids in some embodiments contain TILs. In some embodiments, wherein the disclosure exemplifies pleural fluid, the same methods may be performed with similar results using ascites or other cyst fluids containing TILs.

In some embodiments, the pleural fluid is in unprocessed form, directly as removed from the patient. In some embodiments, the unprocessed pleural fluid is placed in a standard blood collection tube, such as an EDTA or Heparin tube, prior to the contacting step. In some embodiments, the unprocessed pleural fluid is placed in a standard CellSave® tube (Veridex) prior to the contacting step. In some embodiments, the sample is placed in the CellSave tube immediately after collection from the patient to avoid a decrease in the number of viable TILs. The number of viable TILs can decrease to a significant extent within 24 hours, if left in the untreated pleural fluid, even at 4° C. In some embodiments, the sample is placed in the appropriate collection tube within 1 hour, 5 hours, 10 hours, 15 hours, or up to 24 hours after removal from the patient. In some embodiments, the sample is placed in the appropriate collection tube within 1 hour, 5 hours, 10 hours, 15 hours, or up to 24 hours after removal from the patient at 4° C.

In some embodiments, the pleural fluid sample from the chosen subject may be diluted. In some embodiments, the dilution is 1:10 pleural fluid to diluent. In other embodiments, the dilution is 1:9 pleural fluid to diluent. In other embodiments, the dilution is 1:8 pleural fluid to diluent. In other embodiments, the dilution is 1:5 pleural fluid to diluent. In other embodiments, the dilution is 1:2 pleural fluid to diluent. In other embodiments, the dilution is 1:1 pleural fluid to diluent. In some embodiments, diluents include saline, phosphate buffered saline, another buffer or a physiologically acceptable diluent. In some embodiments, the sample is placed in the CellSave tube immediately after collection from the patient and dilution to avoid a decrease in the viable TILs, which may occur to a significant extent within 24-48 hours, if left in the untreated pleural fluid, even at 4° C. In some embodiments, the pleural fluid sample is placed in the appropriate collection tube within 1 hour, 5 hours, 10 hours, 15 hours, 24 hours, 36 hours, up to 48 hours after removal from the patient, and dilution. In some embodiments, the pleural fluid sample is placed in the appropriate collection tube within 1 hour, 5 hours, 10 hours, 15 hours, 24 hours, 36 hours, up to 48 hours after removal from the patient, and dilution at 4° C.

In still other embodiments, pleural fluid samples are concentrated by conventional means prior further processing steps. In some embodiments, this pre-treatment of the pleural fluid is preferable in circumstances in which the pleural fluid must be cryopreserved for shipment to a laboratory performing the method or for later analysis (e.g., later than 24-48 hours post-collection). In some embodiments, the pleural fluid sample is prepared by centrifuging the pleural fluid sample after its withdrawal from the subject and resuspending the centrifugate or pellet in buffer. In some embodiments, the pleural fluid sample is subjected to multiple centrifugations and resuspensions, before it is cryopreserved for transport or later analysis and/or processing.

In some embodiments, pleural fluid samples are concentrated prior to further processing steps by using a filtration method. In some embodiments, the pleural fluid sample used in the contacting step is prepared by filtering the fluid through a filter containing a known and essentially uniform pore size that allows for passage of the pleural fluid through the membrane but retains the tumor cells. In some embodiments, the diameter of the pores in the membrane may be at least 4 μM. In other embodiments the pore diameter may be 5 μM or more, and in other embodiment, any of 6, 7, 8, 9, or 10 μM. After filtration, the cells, including TILs, retained by the membrane may be rinsed off the membrane into a suitable physiologically acceptable buffer. Cells, including TILs, concentrated in this way may then be used in the contacting step of the method.

In some embodiments, pleural fluid sample (including, for example, the untreated pleural fluid), diluted pleural fluid, or the resuspended cell pellet, is contacted with a lytic reagent that differentially lyses non-nucleated red blood cells present in the sample. In some embodiments, this step is performed prior to further processing steps in circumstances in which the pleural fluid contains substantial numbers of RBCs. Suitable lysing reagents include a single lytic reagent or a lytic reagent and a quench reagent, or a lytic agent, a quench reagent and a fixation reagent. Suitable lytic systems are marketed commercially and include the BD Pharm Lyse™ system (Becton Dickenson). Other lytic systems include the Versalyse™ system, the FACSlyse™ system (Becton Dickenson), the Immunoprep™ system or Erythrolyse II system (Beckman Coulter, Inc.), or an ammonium chloride system. In some embodiments, the lytic reagent can vary with the primary requirements being efficient lysis of the red blood cells, and the conservation of the TILs and phenotypic properties of the TILs in the pleural fluid. In addition to employing a single reagent for lysis, the lytic systems useful in methods described herein can include a second reagent, e.g., one that quenches or retards the effect of the lytic reagent during the remaining steps of the method, e.g., Stabilyse™ reagent (Beckman Coulter, Inc.). A conventional fixation reagent may also be employed depending upon the choice of lytic reagents or the preferred implementation of the method.

In some embodiments, the pleural fluid sample, unprocessed, diluted or multiply centrifuged or processed as described herein above is cryopreserved at a temperature of about −140° C. prior to being further processed and/or expanded as provided herein.

3. Methods of Expanding Peripheral Blood Lymphocytes (PBLs) from Peripheral Blood

PBL Method 1. In some embodiments of the invention, PBLs are expanded using the processes described herein. In some embodiments of the invention, the method comprises obtaining a PBMC sample from whole blood. In some embodiments, the method comprises enriching T-cells by isolating pure T-cells from PBMCs using negative selection of a non-CD19+ fraction. In some embodiments, the method comprises enriching T-cells by isolating pure T-cells from PBMCs using magnetic bead-based negative selection of a non-CD19+ fraction.

In some embodiments of the invention, PBL Method 1 is performed as follows: On Day 0, a cryopreserved PBMC sample is thawed and PBMCs are counted. T-cells are isolated using a Human Pan T-Cell Isolation Kit and LS columns (Miltenyi Biotec).

PBL Method 2. In some embodiments of the invention, PBLs are expanded using PBL Method 2, which comprises obtaining a PBMC sample from whole blood. The T-cells from the PBMCs are enriched by incubating the PBMCs for at least three hours at 37° C. and then isolating the non-adherent cells.

In some embodiments of the invention, PBL Method 2 is performed as follows: On Day 0, the cryopreserved PMBC sample is thawed and the PBMC cells are seeded at 6 million cells per well in a 6 well plate in CM-2 media and incubated for 3 hours at 37 degrees Celsius. After 3 hours, the non-adherent cells, which are the PBLs, are removed and counted.

PBL Method 3. In some embodiments of the invention, PBLs are expanded using PBL Method 3, which comprises obtaining a PBMC sample from peripheral blood. B-cells are isolated using a CD19+ selection and T-cells are selected using negative selection of the non-CD19+ fraction of the PBMC sample.

In some embodiments of the invention, PBL Method 3 is performed as follows: On Day 0, cryopreserved PBMCs derived from peripheral blood are thawed and counted. CD19+ B-cells are sorted using a CD19 Multisort Kit, Human (Miltenyi Biotec). Of the non-CD19+ cell fraction, T-cells are purified using the Human Pan T-cell Isolation Kit and LS Columns (Miltenyi Biotec).

In some embodiments, PBMCs are isolated from a whole blood sample. In some embodiments, the PBMC sample is used as the starting material to expand the PBLs. In some embodiments, the sample is cryopreserved prior to the expansion process. In other embodiments, a fresh sample is used as the starting material to expand the PBLs. In some embodiments of the invention, T-cells are isolated from PBMCs using methods known in the art. In some embodiments, the T-cells are isolated using a Human Pan T-cell isolation kit and LS columns. In some embodiments of the invention, T-cells are isolated from PBMCs using antibody selection methods known in the art, for example, CD19 negative selection.

In some embodiments of the invention, the PBMC sample is incubated for a period of time at a desired temperature effective to identify the non-adherent cells. In some embodiments of the invention, the incubation time is about 3 hours. In some embodiments of the invention, the temperature is about 370 Celsius. The non-adherent cells are then expanded using the process described above.

In some embodiments, the PBMC sample is from a subject or patient who has been optionally pre-treated with a regimen comprising a kinase inhibitor or an ITK inhibitor. In some embodiments, the tumor sample is from a subject or patient who has been pre-treated with a regimen comprising a kinase inhibitor or an ITK inhibitor. In some embodiments, the PBMC sample is from a subject or patient who has been pre-treated with a regimen comprising a kinase inhibitor or an ITK inhibitor, has undergone treatment for at least 1 month, at least 2 months, at least 3 months, at least 4 months, at least 5 months, at least 6 months, or 1 year or more. In other embodiments, the PBMCs are derived from a patient who is currently on an ITK inhibitor regimen, such as ibrutinib.

In some embodiments, the PBMC sample is from a subject or patient who has been pre-treated with a regimen comprising a kinase inhibitor or an ITK inhibitor and is refractory to treatment with a kinase inhibitor or an ITK inhibitor, such as ibrutinib.

In some embodiments, the PBMC sample is from a subject or patient who has been pre-treated with a regimen comprising a kinase inhibitor or an ITK inhibitor but is no longer undergoing treatment with a kinase inhibitor or an ITK inhibitor. In some embodiments, the PBMC sample is from a subject or patient who has been pre-treated with a regimen comprising a kinase inhibitor or an ITK inhibitor but is no longer undergoing treatment with a kinase inhibitor or an ITK inhibitor and has not undergone treatment for at least 1 month, at least 2 months, at least 3 months, at least 4 months, at least 5 months, at least 6 months, or at least 1 year or more. In other embodiments, the PBMCs are derived from a patient who has prior exposure to an ITK inhibitor, but has not been treated in at least 3 months, at least 6 months, at least 9 months, or at least 1 year.

In some embodiments of the invention, at Day 0, cells are selected for CD19+ and sorted accordingly. In some embodiments of the invention, the selection is made using antibody binding beads. In some embodiments of the invention, pure T-cells are isolated on Day 0 from the PBMCs.

In some embodiments of the invention, for patients that are not pre-treated with ibrutinib or other ITK inhibitor, 10-15 mL of Buffy Coat will yield about 5×10⁹ PBMC, which, in turn, will yield about 5.5×10⁷ PBLs.

In some embodiments of the invention, for patients that are pre-treated with ibrutinib or other ITK inhibitor, the expansion process will yield about 20×10⁹ PBLs. In some embodiments of the invention, 40.3×10⁶ PBMCs will yield about 4.7×10⁵ PBLs.

In any of the foregoing embodiments, PBMCs may be derived from a whole blood sample, by apheresis, from the buffy coat, or from any other method known in the art for obtaining PBMCs.

In some embodiments, PBLs are prepared using the methods described in U.S. Patent Application Publication No. US 2020/0347350 A1, the disclosures of which are incorporated by reference herein.

4. Methods of Expanding Marrow Infiltrating Lymphocytes (MILs) from PBMCs Derived from Bone Marrow

MIL Method 3. In some embodiments of the invention, the method comprises obtaining PBMCs from the bone marrow. On Day 0, the PBMCs are selected for CD3+/CD33+/CD20+/CD14+ and sorted, and the non-CD3+/CD33+/CD20+/CD14+ cell fraction is sonicated and a portion of the sonicated cell fraction is added back to the selected cell fraction.

In some embodiments of the invention, MIL Method 3 is performed as follows: On Day 0, a cryopreserved sample of PBMCs is thawed and PBMCs are counted. The cells are stained with CD3, CD33, CD20, and CD14 antibodies and sorted using a S3e cell sorted (Bio-Rad). The cells are sorted into two fractions—an immune cell fraction (or the MIL fraction) (CD3+CD33+CD20+CD14+) and an AML blast cell fraction (non-CD3+CD33+CD20+CD14+).

In some embodiments of the invention, PBMCs are obtained from bone marrow. In some embodiments, the PBMCs are obtained from the bone marrow through apheresis, aspiration, needle biopsy, or other similar means known in the art. In some embodiments, the PBMCs are fresh. In other embodiments, the PBMCs are cryopreserved.

In some embodiments of the invention, MILs are expanded from 10-50 mL of bone marrow aspirate. In some embodiments of the invention, 10 mL of bone marrow aspirate is obtained from the patient. In other embodiments, 20 mL of bone marrow aspirate is obtained from the patient. In other embodiments, 30 mL of bone marrow aspirate is obtained from the patient. In other embodiments, 40 mL of bone marrow aspirate is obtained from the patient. In other embodiments, 50 mL of bone marrow aspirate is obtained from the patient.

In some embodiments of the invention, the number of PBMCs yielded from about 10-50 mL of bone marrow aspirate is about 5×10⁷ to about 10×10⁷ PBMCs. In other embodiments, the number of PMBCs yielded is about 7×10⁷ PBMCs.

In some embodiments of the invention, about 5×10⁷ to about 10×10⁷ PBMCs, yields about 0.5×10⁶ to about 1.5×10⁶ MILs. In some embodiments of the invention, about 1×10⁶ MILs is yielded.

In some embodiments of the invention, 12×10⁶ PBMC derived from bone marrow aspirate yields approximately 1.4×10⁵ MILs.

In any of the foregoing embodiments, PBMCs may be derived from a whole blood sample, from bone marrow, by apheresis, from the buffy coat, or from any other method known in the art for obtaining PBMCs.

In some embodiments, MILs are prepared using the methods described in U.S. Patent Application Publication No. US 2020/0347350 A1, the disclosures of which are incorporated by reference herein.

### B. STEP B: Priming First Expansion

In some embodiments, the present methods provide for younger TILs, which may provide additional therapeutic benefits over older TILs (i.e., TILs which have further undergone more rounds of replication prior to administration to a subject/patient). Features of young TILs have been described in the literature, for example in Donia, et al., Scand. J. Immunol. 2012, 75, 157-167; Dudley, et al., Clin. Cancer Res. 2010, 16, 6122-6131; Huang, et al., J. Immunother. 2005, 28, 258-267; Besser, et al., Clin. Cancer Res. 2013, 19, OF1-OF9; Besser, et al., J. Immunother. 2009, 32, 415-423; Robbins, et al., J. Immunol. 2004, 173, 7125-7130; Shen, et al., J. Immunother., 2007, 30, 123-129; Zhou, et al., J. Immunother. 2005, 28, 53-62; and Tran, et al., J Immunother., 2008, 31, 742-751, each of which is incorporated herein by reference.

After dissection or digestion of tumor fragments and/or tumor fragments, for example such as described in Step A of FIG. 8 (in particular, e.g., FIG. 8A and/or FIG. 8B and/or FIG. 8C), the resulting cells are cultured in serum containing IL-2, OKT-3, and feeder cells (e.g., antigen-presenting feeder cells), under conditions that favor the growth of TILs over tumor and other cells. In some embodiments, the IL-2, OKT-3, and feeder cells are added at culture initiation along with the tumor digest and/or tumor fragments (e.g., at Day 0). In some embodiments, the tumor digests and/or tumor fragments are incubated in a container with up to 60 fragments per container and with 6000 IU/mL of IL-2. In some embodiments, this primary cell population is cultured for a period of days, generally from 1 to 8 days, resulting in a bulk TIL population, generally about 1×10⁸ bulk TIL cells. In some embodiments, this primary cell population is cultured for a period of days, generally from 1 to 7 days, resulting in a bulk TIL population, generally about 1×10⁸ bulk TIL cells. In some embodiments, priming first expansion occurs for a period of 1 to 8 days, resulting in a bulk TIL population, generally about 1×10⁸ bulk TIL cells. In some embodiments, priming first expansion occurs for a period of 1 to 7 days, resulting in a bulk TIL population, generally about 1×10⁸ bulk TIL cells. In some embodiments, this priming first expansion occurs for a period of 5 to 8 days, resulting in a bulk TIL population, generally about 1×10⁸ bulk TIL cells. In some embodiments, this priming first expansion occurs for a period of 5 to 7 days, resulting in a bulk TIL population, generally about 1×10⁸ bulk TIL cells. In some embodiments, this priming first expansion occurs for a period of about 6 to 8 days, resulting in a bulk TIL population, generally about 1×10⁸ bulk TIL cells. In some embodiments, this priming first expansion occurs for a period of about 6 to 7 days, resulting in a bulk TIL population, generally about 1×10⁸ bulk TIL cells. In some embodiments, this priming first expansion occurs for a period of about 7 to 8 days, resulting in a bulk TIL population, generally about 1×10⁸ bulk TIL cells. In some embodiments, this priming first expansion occurs for a period of about 7 days, resulting in a bulk TIL population, generally about 1×10⁸ bulk TIL cells. In some embodiments, this priming first expansion occurs for a period of about 8 days, resulting in a bulk TIL population, generally about 1×10⁸ bulk TIL cells.

In some embodiments, expansion of TILs may be performed using a priming first expansion step (for example such as those described in Step B of FIG. 8 (in particular, e.g., FIG. 8A and/or FIG. 8B and/or FIG. 8C and/or FIG. 8D), which can include processes referred to as pre-REP or priming REP and which contains feeder cells from Day 0 and/or from culture initiation) as described below and herein, followed by a rapid second expansion (Step D, including processes referred to as rapid expansion protocol (REP) steps) as described below under Step D and herein, followed by optional cryopreservation, and followed by a second Step D (including processes referred to as restimulation REP steps) as described below and herein. The TILs obtained from this process may be optionally characterized for phenotypic characteristics and metabolic parameters as described herein. In some embodiments, the tumor fragment is between about 1 mm³ and 10 mm³.

In some embodiments, the first expansion culture medium is referred to as “CM”, an abbreviation for culture media. In some embodiments, CM for Step B consists of RPMI 1640 with GlutaMAX, supplemented with 10% human AB serum, 25 mM Hepes, and 10 mg/mL gentamicin.

In some embodiments, there are less than or equal to 240 tumor fragments. In some embodiments, there are less than or equal to 240 tumor fragments placed in less than or equal to 4 containers. In some embodiments, the containers are GREX100 MCS flasks. In some embodiments, less than or equal to 60 tumor fragments are placed in 1 container. In some embodiments, each container comprises less than or equal to 500 mL of media per container. In some embodiments, the media comprises IL-2. In some embodiments, the media comprises 6000 IU/mL of IL-2. In some embodiments, the media comprises antigen-presenting feeder cells (also referred to herein as “antigen-presenting cells”). In some embodiments, the media comprises 2.5×10⁸ antigen-presenting feeder cells per container. In some embodiments, the media comprises OKT-3. In some embodiments, the media comprises 30 ng/mL of OKT-3 per container. In some embodiments, the container is a GREX100 MCS flask. In some embodiments, the media comprises 6000 IU/mL of IL-2, 30 ng of OKT-3, and 2.5×10⁸ antigen-presenting feeder cells. In some embodiments, the media comprises 6000 IU/mL of IL-2, 30 ng/mL of OKT-3, and 2.5×10⁸ antigen-presenting feeder cells per container.

After preparation of the tumor fragments, the resulting cells (i.e., fragments which is a primary cell population) are cultured in media containing IL-2, antigen-presenting feeder cells and OKT-3 under conditions that favor the growth of TILs over tumor and other cells and which allow for TIL priming and accelerated growth from initiation of the culture on Day 0. In some embodiments, the tumor digests and/or tumor fragments are incubated in with 6000 IU/mL of IL-2, as well as antigen-presenting feeder cells and OKT-3. This primary cell population is cultured for a period of days, generally from 1 to 8 days, resulting in a bulk TIL population, generally about 1×10⁸ bulk TIL cells. In some embodiments, the growth media during the priming first expansion comprises IL-2 or a variant thereof, as well as antigen-presenting feeder cells and OKT-3. In some embodiments, this primary cell population is cultured for a period of days, generally from 1 to 7 days, resulting in a bulk TIL population, generally about 1×10⁸ bulk TIL cells. In some embodiments, the growth media during the priming first expansion comprises IL-2 or a variant thereof, as well as antigen-presenting feeder cells and OKT-3. In some embodiments, the IL-2 is recombinant human IL-2 (rhIL-2). In some embodiments the IL-2 stock solution has a specific activity of 20-30×10⁶ IU/mg for a 1 mg vial. In some embodiments the IL-2 stock solution has a specific activity of 20×10⁶ IU/mg for a 1 mg vial. In some embodiments the IL-2 stock solution has a specific activity of 25×10⁶ IU/mg for a 1 mg vial. In some embodiments the IL-2 stock solution has a specific activity of 30×10⁶ IU/mg for a 1 mg vial. In some embodiments, the IL-2 stock solution has a final concentration of 4-8×10⁶ IU/mg of IL-2. In some embodiments, the IL-2 stock solution has a final concentration of 5-7×10⁶ IU/mg of IL-2. In some embodiments, the IL-2 stock solution has a final concentration of 6×10⁶ IU/mg of IL-2. In some embodiments, the IL-2 stock solution is prepare as described in Example C. In some embodiments, the priming first expansion culture media comprises about 10,000 IU/mL of IL-2, about 9,000 IU/mL of IL-2, about 8,000 IU/mL of IL-2, about 7,000 IU/mL of IL-2, about 6000 IU/mL of IL-2 or about 5,000 IU/mL of IL-2. In some embodiments, the priming first expansion culture media comprises about 9,000 IU/mL of IL-2 to about 5,000 IU/mL of IL-2. In some embodiments, the priming first expansion culture media comprises about 8,000 IU/mL of IL-2 to about 6,000 IU/mL of IL-2. In some embodiments, the priming first expansion culture media comprises about 7,000 IU/mL of IL-2 to about 6,000 IU/mL of IL-2. In some embodiments, the priming first expansion culture media comprises about 6,000 IU/mL of IL-2. In some embodiments, the cell culture medium further comprises IL-2. In some embodiments, the priming first expansion cell culture medium comprises about 3000 IU/mL of IL-2. In some embodiments, the priming first expansion cell culture medium further comprises IL-2. In some embodiments, the priming first expansion cell culture medium comprises about 3000 IU/mL of IL-2. In some embodiments, the priming first expansion cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In some embodiments, the priming first expansion cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or about 8000 IU/mL of IL-2.

In some embodiments, priming first expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the priming first expansion culture media comprises about 500 IU/mL of IL-15 to about 100 IU/mL of IL-15. In some embodiments, the priming first expansion culture media comprises about 400 IU/mL of IL-15 to about 100 IU/mL of IL-15. In some embodiments, the priming first expansion culture media comprises about 300 IU/mL of IL-15 to about 100 IU/mL of IL-15. In some embodiments, the priming first expansion culture media comprises about 200 IU/mL of IL-15. In some embodiments, the priming first expansion cell culture medium comprises about 180 IU/mL of IL-15. In some embodiments, the priming first expansion cell culture medium further comprises IL-15. In some embodiments, the priming first expansion cell culture medium comprises about 180 IU/mL of IL-15.

In some embodiments, priming first expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the priming first expansion culture media comprises about 20 IU/mL of IL-21 to about 0.5 IU/mL of IL-21. In some embodiments, the priming first expansion culture media comprises about 15 IU/mL of IL-21 to about 0.5 IU/mL of IL-21. In some embodiments, the priming first expansion culture media comprises about 12 IU/mL of IL-21 to about 0.5 IU/mL of IL-21. In some embodiments, the priming first expansion culture media comprises about 10 IU/mL of IL-21 to about 0.5 IU/mL of IL-21. In some embodiments, the priming first expansion culture media comprises about 5 IU/mL of IL-21 to about 1 IU/mL of IL-21. In some embodiments, the priming first expansion culture media comprises about 2 IU/mL of IL-21. In some embodiments, the priming first expansion cell culture medium comprises about 1 IU/mL of IL-21. In some embodiments, the priming first expansion cell culture medium comprises about 0.5 IU/mL of IL-21. In some embodiments, the cell culture medium further comprises IL-21. In some embodiments, the priming first expansion cell culture medium comprises about 1 IU/mL of IL-21.

In some embodiments, the priming first expansion cell culture medium comprises OKT-3 antibody. In some embodiments, the priming first expansion cell culture medium comprises about 30 ng/mL of OKT-3 antibody. In some embodiments, the priming first expansion cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In some embodiments, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium comprises between 15 ng/mL and 30 ng/mL of OKT-3 antibody. In some embodiments, the cell culture medium comprises 30 ng/mL of OKT-3 antibody. In some embodiments, the OKT-3 antibody is muromonab. See, for example, Table 1.

In some embodiments, the priming first expansion cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the priming first expansion cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist. In some embodiments, in addition to one or more TNFRSF agonists, the priming first expansion cell culture medium further comprises IL-2 at an initial concentration of about 6000 IU/mL and OKT-3 antibody at an initial concentration of about 30 ng/mL, and wherein the one or more TNFRSF agonists comprises a 4-1BB agonist.

In some embodiments, the priming first expansion culture medium is referred to as “CM”, an abbreviation for culture media. In some embodiments, it is referred to as CM1 (culture medium 1). In some embodiments, CM consists of RPMI 1640 with GlutaMAX, supplemented with 10% human AB serum, 25 mM Hepes, and 10 mg/mL gentamicin. In some embodiments, the CM is the CM1 described in the Examples. In some embodiments, the priming first expansion occurs in an initial cell culture medium or a first cell culture medium. In some embodiments, the priming first expansion culture medium or the initial cell culture medium or the first cell culture medium comprises IL-2, OKT-3 and antigen-presenting feeder cells (also referred to herein as feeder cells).

In some embodiments, the culture medium used in the expansion processes disclosed herein is a serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium, CTS™ OpTmizer™ T-Cell Expansion SFM, CTS™ AIM-V Medium, CTS™ ATM-V SFM, LymphoONE™ T-Cell Expansion Xeno-Free Medium, Dulbecco's Modified Eagle's Medium (DMEM1) Minimal Essential Medium (MEM), Basal Medium Eagle (BMEV RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CST™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (uMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%, 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific). In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 μM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2. In some embodiments, the CTS™ OpTmizer™ T-cell Expansion SFM is supplemented with about 3% of the CTS™ Immune Cell Serum Replacement (SR) (ThermoFisher Scientific) and the final concentration of 2-mercaptoethanol in the media is 55 μM.

In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM. In some embodiments, the final concentration of 2-mercaptoethanol in the media is 55 μM.

In some embodiments, the defined media described in International PCT Publication No. WO/1998/030679, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, one or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the basal cell media is selected from the group consisting of Dulbecco's Modified Eagle's Medium (DMEM), Minimal Essential Medium (MEM), Basal Medium Eagle (BME), RPMI 1640, F-10, F-12, Minimal Essential Medium (uMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., AlbuMAX® I) is about 5000-50,000 mg/L.

In some embodiments, the non-trace element moiety ingredients in the defined medium are present in the concentration ranges listed in the column under the heading “Concentration Range in 1× Medium” in Table 12. In other embodiments, the non-trace element moiety ingredients in the defined medium are present in the final concentrations listed in the column under the heading “A Preferred Embodiment of the 1×Medium” in Table 12. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 12.

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 M).

In some embodiments, the defined media described in Smith, et al., Clin. Transl. Immunology, 4(1), 2015 (doi: 10.1038/cti.2014.31) are useful in the present invention. Briefly, RPMI or CTS™ OpTmizer™ was used as the basal cell medium, and supplemented with either 0, 2%, 5%, or 10% CTS™ Immune Cell Serum Replacement.

In some embodiments, the cell medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME or OME; also known as 2-mercaptoethanol, CAS 60-24-2).

In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 1 to 8 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 2 to 8 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 3 to 8 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 4 to 8 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 5 to 8 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 6 to 8 days, as discussed in the examples and figures. In some embodiments, the priming first expansion (including processes such as for example those provided in Step B of FIG. 1 (in particular, e.g., FIG. 8A and/or FIG. 8B and/or FIG. 8C and/or FIG. 8D), which can include those sometimes referred to as the pre-REP or priming REP) process is 7 to 8 days, as discussed in the examples and figures. In some embodiments, the priming first expansion (including processes such as for example those provided in Step B of FIG. 8 (in particular, e.g., FIG. 8A and/or FIG. 8B and/or FIG. 8C and/or FIG. 8D), which can include those sometimes referred to as the pre-REP or priming REP) process is 8 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 1 to 7 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 2 to 7 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 3 to 7 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 4 to 7 days, as discussed in the examples and figures. In some embodiments, the priming first expansion (including processes such as for example those described in Step B of FIG. 8 (in particular, e.g., FIG. 8B and/or FIG. 8C), which can include those sometimes referred to as the pre-REP or priming REP) process is 5 to 7 days, as discussed in the examples and figures. In some embodiments, the priming first expansion (including processes such as for example those described in Step B of FIG. 8 (in particular, e.g., FIG. 8A and/or FIG. 8B and/or FIG. 8C and/or FIG. 8D), which can include those sometimes referred to as the pre-REP or priming REP) process is 6 to 7 days, as discussed in the examples and figures. In some embodiments, the priming first expansion (including processes such as for example those provided in Step B of FIG. 8 (in particular, e.g., FIG. 8A and/or FIG. 8B and/or FIG. 8C and/or FIG. 8D), which can include those sometimes referred to as the pre-REP or priming REP) process is 7 days, as discussed in the examples and figures.

In some embodiments, the priming first TIL expansion can proceed for 1 days to 8 days from when fragmentation occurs and/or when the first priming expansion step is initiated. In some embodiments, the priming first TIL expansion can proceed for 1 days to 7 days from when fragmentation occurs and/or when the first priming expansion step is initiated. In some embodiments, the priming first TIL expansion can proceed for 2 days to 8 days from when fragmentation occurs and/or when the first priming expansion step is initiated. In some embodiments, the priming first TIL expansion can proceed for 2 days to 7 days from when fragmentation occurs and/or when the first priming expansion step is initiated. In some embodiments, the priming first TIL expansion can proceed for 3 days to 8 days from when fragmentation occurs and/or when the first priming expansion step is initiated. In some embodiments, the priming first TIL expansion can proceed for 3 days to 7 days from when fragmentation occurs and/or when the first priming expansion step is initiated. In some embodiments, the priming first TIL expansion can proceed for 4 days to 8 days from when fragmentation occurs and/or when the first priming expansion step is initiated. In some embodiments, the priming first TIL expansion can proceed for 4 days to 7 days from when fragmentation occurs and/or when the first priming expansion step is initiated. In some embodiments, the priming first TIL expansion can proceed for 5 days to 8 days from when fragmentation occurs and/or when the first priming expansion step is initiated. In some embodiments, the priming first TIL expansion can proceed for 5 days to 7 days from when fragmentation occurs and/or when the first priming expansion step is initiated. In some embodiments, the priming first TIL expansion can proceed for 6 days to 8 days from when fragmentation occurs and/or when the first priming expansion step is initiated. In some embodiments, the priming first TIL expansion can proceed for 6 days to 7 days from when fragmentation occurs and/or when the first priming expansion step is initiated. In some embodiments, the priming first TIL expansion can proceed for 7 to 8 days from when fragmentation occurs and/or when the first priming expansion step is initiated. In some embodiments, the priming first TIL expansion can proceed for 8 days from when fragmentation occurs and/or when the first priming expansion step is initiated. In some embodiments, the priming first TIL expansion can proceed for 7 days from when fragmentation occurs and/or when the first priming expansion step is initiated.

In some embodiments, the priming first expansion of the TILs can proceed for 1 day, 2 days, 3 days, 4 days, 5 days, 6 days, 7 days, or 8 days. In some embodiments, the first TIL expansion can proceed for 1 day to 8 days. In some embodiments, the first TIL expansion can proceed for 1 day to 7 days. In some embodiments, the first TIL expansion can proceed for 2 days to 8 days. In some embodiments, the first TIL expansion can proceed for 2 days to 7 days. In some embodiments, the first TIL expansion can proceed for 3 days to 8 days. In some embodiments, the first TIL expansion can proceed for 3 days to 7 days. In some embodiments, the first TIL expansion can proceed for 4 days to 8 days. In some embodiments, the first TIL expansion can proceed for 4 days to 7 days. In some embodiments, the first TIL expansion can proceed for 5 days to 8 days. In some embodiments, the first TIL expansion can proceed for 5 days to 7 days. In some embodiments, the first TIL expansion can proceed for 6 days to 8 days. In some embodiments, the first TIL expansion can proceed for 6 days to 7 days. In some embodiments, the first TIL expansion can proceed for 7 to 8 days. In some embodiments, the first TIL expansion can proceed for 8 days. In some embodiments, the first TIL expansion can proceed for 7 days.

In some embodiments, a combination of IL-2, IL-7, IL-15, and/or IL-21 are employed as a combination during the priming first expansion. In some embodiments, IL-2, IL-7, IL-15, and/or IL-21 as well as any combinations thereof can be included during the priming first expansion, including, for example during Step B processes according to FIG. 8 (in particular, e.g., FIG. 8A and/or FIG. 8B and/or FIG. 8C and/or FIG. 8D), as well as described herein. In some embodiments, a combination of IL-2, IL-15, and IL-21 are employed as a combination during the priming first expansion. In some embodiments, IL-2, IL-15, and IL-21 as well as any combinations thereof can be included during Step B processes according to FIG. 8 (in particular, e.g., FIG. 8A and/or FIG. 8B and/or FIG. 8C and/or FIG. 8D) and as described herein.

In some embodiments, the priming first expansion, for example, Step B according to FIG. 8 (in particular, e.g., FIG. 8A and/or FIG. 8B and/or FIG. 8C and/or FIG. 8D), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-10 or a G-REX-100. In some embodiments, the bioreactor employed is a G-REX-100. In some embodiments, the bioreactor employed is a G-REX-10.

### 1. Feeder Cells and Antigen Presenting Cells

In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during days 4-8. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during days 4-7. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during days 5-8. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during days 5-7. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during days 6-8. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during days 6-7. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during day 7 or 8. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during day 7. In some embodiments, the priming first expansion procedures described herein (for example including expansion such as those described in Step B from FIG. 8 (in particular, e.g., FIG. 8A and/or FIG. 8B and/or FIG. 8C and/or FIG. 8D), as well as those referred to as pre-REP or priming REP) does not require feeder cells (also referred to herein as “antigen-presenting cells”) at the initiation of the TIL expansion, but rather are added during the priming first expansion at any time during day 8.

In some embodiments, the priming first expansion procedures described herein (for example including expansion such as those described in Step B from FIG. 8 (in particular, e.g., FIG. 8B), as well as those referred to as pre-REP or priming REP) require feeder cells (also referred to herein as “antigen-presenting cells”) at the initiation of the TIL expansion and during the priming first expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In some embodiments, 2.5×10⁸ feeder cells are used during the priming first expansion. In some embodiments, 2.5×10⁸ feeder cells per container are used during the priming first expansion. In some embodiments, 2.5×10⁸ feeder cells per GREX-10 are used during the priming first expansion. In some embodiments, 2.5×10⁸ feeder cells per GREX-100 are used during the priming first expansion.

In general, the allogeneic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and acceptable for use in the TIL expansion procedures described herein if the total number of viable cells on day 14 is less than the initial viable cell number put into culture on day 0 of the priming first expansion.

In some embodiments, PBMCs are considered replication incompetent and acceptable for use in the TIL expansion procedures described herein if the total number of viable cells, cultured in the presence of OKT3 and IL-2, on day 7 have not increased from the initial viable cell number put into culture on day 0 of the priming first expansion. In some embodiments, the PBMCs are cultured in the presence of 30 ng/mL OKT3 antibody and 3000 IU/mL IL-2. In some embodiments, the PBMCs are cultured in the presence of 30 ng/mL OKT3 antibody and 6000 IU/mL IL-2.

In some embodiments, PBMCs are considered replication incompetent and acceptable for use in the TIL expansion procedures described herein if the total number of viable cells, cultured in the presence of OKT3 and IL-2, on day 7 have not increased from the initial viable cell number put into culture on day 0 of the priming first expansion. In some embodiments, the PBMCs are cultured in the presence of 5-60 ng/mL OKT3 antibody and 1000-6000 IU/mL IL-2. In some embodiments, the PBMCs are cultured in the presence of 10-50 ng/mL OKT3 antibody and 2000-5000 IU/mL IL-2. In some embodiments, the PBMCs are cultured in the presence of 20-40 ng/mL OKT3 antibody and 2000-4000 IU/mL IL-2. In some embodiments, the PBMCs are cultured in the presence of 25-35 ng/mL OKT3 antibody and 2500-3500 IU/mL IL-2. In some embodiments, the PBMCs are cultured in the presence of 30 ng/mL OKT3 antibody and 6000 IU/mL IL-2. In some embodiments, the PBMCs are cultured in the presence of 15 ng/mL OKT3 antibody and 3000 IU/mL IL-2. In some embodiments, the PBMCs are cultured in the presence of 15 ng/mL OKT3 antibody and 6000 IU/mL IL-2.

In some embodiments, the antigen-presenting feeder cells are PBMCs. In some embodiments, the antigen-presenting feeder cells are artificial antigen-presenting feeder cells. In some embodiments, the ratio of TILs to antigen-presenting feeder cells in the second expansion is about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to antigen-presenting feeder cells in the second expansion is between 1 to 50 and 1 to 300. In some embodiments, the ratio of TILs to antigen-presenting feeder cells in the second expansion is between 1 to 100 and 1 to 200.

In some embodiments, the priming first expansion procedures described herein require a ratio of about 2.5×10⁸ feeder cells to about 100×10⁶ TILs. In other embodiments, the priming first expansion procedures described herein require a ratio of about 2.5×10⁸ feeder cells to about 50×10⁶ TILs. In yet other embodiments, the priming first expansion described herein require about 2.5×10⁸ feeder cells to about 25×10⁶ TILs. In yet other embodiments, the priming first expansion described herein require about 2.5×10⁸ feeder cells. In yet other embodiments, the priming first expansion requires one-fourth, one-third, five-twelfths, or one-half of the number of feeder cells used in the rapid second expansion.

In some embodiments, the media in the priming first expansion comprises IL-2. In some embodiments, the media in the priming first expansion comprises 6000 IU/mL of IL-2. In some embodiments, the media in the priming first expansion comprises antigen-presenting feeder cells. In some embodiments, the media in the priming first expansion comprises 2.5×10⁸ antigen-presenting feeder cells per container. In some embodiments, the media in the priming first expansion comprises OKT-3. In some embodiments, the media comprises 30 ng of OKT-3 per container. In some embodiments, the container is a GREX100 MCS flask. In some embodiments, the media comprises 6000 IU/mL of IL-2, 30 ng/mL of OKT-3, and 2.5×10⁸ antigen-presenting feeder cells. In some embodiments, the media comprises 6000 IU/mL of IL-2, 30 ng/mL of OKT-3, and 2.5×10⁸ antigen-presenting feeder cells per container. In some embodiments, the media comprises 500 mL of culture medium and 15 μg of OKT-3 per 2.5×10⁸ antigen-presenting feeder cells per container. In some embodiments, the media comprises 500 mL of culture medium and 15 μg of OKT-3 per container. In some embodiments, the container is a GREX100 MCS flask. In some embodiments, the media comprises 500 mL of culture medium, 6000 IU/mL of IL-2, 30 ng/mL of OKT-3, and 2.5×10⁸ antigen-presenting feeder cells. In some embodiments, the media comprises 500 mL of culture medium, 6000 IU/mL of IL-2, 15 μg of OKT-3, and 2.5×10⁸ antigen-presenting feeder cells per container. In some embodiments, the media comprises 500 mL of culture medium and 15 μg of OKT-3 per 2.5×10⁸ antigen-presenting feeder cells per container.

In some embodiments, the priming first expansion procedures described herein require an excess of feeder cells over TILs during the second expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In some embodiments, artificial antigen-presenting (aAPC) cells are used in place of PBMCs.

In general, the allogeneic PBMCs are inactivated, either via irradiation or heat treatment, and used in the TIL expansion procedures described herein, including the exemplary procedures described in the figures and examples.

In some embodiments, artificial antigen presenting cells are used in the priming first expansion as a replacement for, or in combination with, PBMCs.

### 2. Cytokines and Other Additives

The expansion methods described herein generally use culture media with high doses of a cytokine, in particular IL-2, as is known in the art.

Alternatively, using combinations of cytokines for the priming first expansion of TILs is additionally possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21, and IL-2, IL-15 and IL-21, with the latter finding particular use in many embodiments. The use of combinations of cytokines specifically favors the generation of lymphocytes, and in particular T-cells as described therein. See, for example, Table 2.

In some embodiments, Step B may also include the addition of OKT-3 antibody or muromonab to the culture media, as described elsewhere herein. In some embodiments, Step B may also include the addition of a 4-1BB agonist to the culture media, as described elsewhere herein. In some embodiments, Step B may also include the addition of an OX-40 agonist to the culture media, as described elsewhere herein. In addition, additives such as peroxisome proliferator-activated receptor gamma coactivator I-alpha agonists, including proliferator-activated receptor (PPAR)-gamma agonists such as a thiazolidinedione compound, may be used in the culture media during Step B, as described in U.S. Patent Application Publication No. US 2019/0307796 A1, the disclosure of which is incorporated by reference herein.

### C. STEP C: Priming First Expansion to Rapid Second Expansion Transition

In some cases, the bulk TIL population obtained from the priming first expansion (which can include expansions sometimes referred to as pre-REP), including, for example the TIL population obtained from for example, Step B as indicated in FIG. 8 (in particular, e.g., FIG. 8A and/or FIG. 8B and/or FIG. 8C and/or FIG. 8D), can be subjected to a rapid second expansion (which can include expansions sometimes referred to as Rapid Expansion Protocol (REP)) and then cryopreserved as discussed below. Similarly, in the case where genetically modified TILs will be used in therapy, the expanded TIL population from the priming first expansion or the expanded TIL population from the rapid second expansion can be subjected to genetic modifications for suitable treatments prior to the expansion step or after the priming first expansion and prior to the rapid second expansion.

In some embodiments, the TILs obtained from the priming first expansion (for example, from Step B as indicated in FIG. 8 (in particular, e.g., FIG. 8A and/or FIG. 8B and/or FIG. 8C and/or FIG. 8D)) are stored until phenotyped for selection. In some embodiments, the TILs obtained from the priming first expansion (for example, from Step B as indicated in FIG. 8 (in particular, e.g., FIG. 8A and/or FIG. 8B and/or FIG. 8C and/or FIG. 8D)) are not stored and proceed directly to the rapid second expansion. In some embodiments, the TILs obtained from the priming first expansion are not cryopreserved after the priming first expansion and prior to the rapid second expansion. In some embodiments, the transition from the priming first expansion to the second expansion occurs at about 2 days, 3 days, 4, days, 5 days, 6 days, 7 days, or 8 days from when tumor fragmentation occurs and/or when the first priming expansion step is initiated. In some embodiments, the transition from the priming first expansion to the rapid second expansion occurs at about 3 days to 7 days from when fragmentation occurs and/or when the first priming expansion step is initiated. In some embodiments, the transition from the priming first expansion to the rapid second expansion occurs at about 3 days to 8 days from when fragmentation occurs and/or when the first priming expansion step is initiated. In some embodiments, the transition from the priming first expansion to the second expansion occurs at about 4 days to 7 days from when fragmentation occurs and/or when the first priming expansion step is initiated. In some embodiments, the transition from the priming first expansion to the second expansion occurs at about 4 days to 8 days from when fragmentation occurs and/or when the first priming expansion step is initiated. In some embodiments, the transition from the priming first expansion to the second expansion occurs at about 5 days to 7 days from when fragmentation occurs and/or when the first priming expansion step is initiated. In some embodiments, the transition from the priming first expansion to the second expansion occurs at about 5 days to 8 days from when fragmentation occurs and/or when the first priming expansion step is initiated. In some embodiments, the transition from the priming first expansion to the second expansion occurs at about 6 days to 7 days from when fragmentation occurs and/or when the first priming expansion step is initiated. In some embodiments, the transition from the priming first expansion to the second expansion occurs at about 6 days to 8 days from when fragmentation occurs and/or when the first priming expansion step is initiated. In some embodiments, the transition from the priming first expansion to the second expansion occurs at about 7 days to 8 days from when fragmentation occurs and/or when the first priming expansion step is initiated. In some embodiments, the transition from the priming first expansion to the second expansion occurs at about 7 days from when fragmentation occurs and/or when the first priming expansion step is initiated. In some embodiments, the transition from the priming first expansion to the second expansion occurs at about 8 days from when fragmentation occurs and/or when the first priming expansion step is initiated.

In some embodiments, the transition from the priming first expansion to the rapid second expansion occurs at 1 day, 2 days, 3 days, 4 days, 5 days, 6 days, 7 days, or 8 days from when fragmentation occurs and/or when the first priming expansion step is initiated. In some embodiments, the transition from the priming first expansion to the rapid second expansion occurs 1 day to 7 days from when fragmentation occurs and/or when the first priming expansion step is initiated. In some embodiments, the transition from the priming first expansion to the rapid second expansion occurs 1 day to 8 days from when fragmentation occurs and/or when the first priming expansion step is initiated. In some embodiments, the transition from the priming first expansion to the second expansion occurs 2 days to 7 days from when fragmentation occurs and/or when the first priming expansion step is initiated. In some embodiments, the transition from the priming first expansion to the second expansion occurs 2 days to 8 days from when fragmentation occurs and/or when the first priming expansion step is initiated. In some embodiments, the transition from the priming first expansion to the second expansion occurs 3 days to 7 days from when fragmentation occurs and/or when the first priming expansion step is initiated. In some embodiments, the transition from the priming first expansion to the second expansion occurs 3 days to 8 days from when fragmentation occurs and/or when the first priming expansion step is initiated. In some embodiments, the transition from the priming first expansion to the rapid second expansion occurs 4 days to 7 days from when fragmentation occurs and/or when the first priming expansion step is initiated. In some embodiments, the transition from the priming first expansion to the rapid second expansion occurs 4 days to 8 days from when fragmentation occurs and/or when the first priming expansion step is initiated. In some embodiments, the transition from the priming first expansion to the rapid second expansion occurs 5 days to 7 days from when fragmentation occurs and/or when the first priming expansion step is initiated. In some embodiments, the transition from the priming first expansion to the rapid second expansion occurs 5 days to 8 days from when fragmentation occurs and/or when the first priming expansion step is initiated. In some embodiments, the transition from the priming first expansion to the rapid second expansion occurs 6 days to 7 days from when fragmentation occurs and/or when the first priming expansion step is initiated. In some embodiments, the transition from the priming first expansion to the rapid second expansion occurs 6 days to 8 days from when fragmentation occurs and/or when the first priming expansion step is initiated. In some embodiments, the transition from the priming first expansion to the rapid second expansion occurs 7 days to 8 days from when fragmentation occurs and/or when the first priming expansion step is initiated. In some embodiments, the transition from the priming first expansion to the rapid second expansion occurs 7 days from when fragmentation occurs and/or when the first priming expansion step is initiated. In some embodiments, the transition from the priming first expansion to the rapid second expansion occurs 8 days from when fragmentation occurs and/or when the first priming expansion step is initiated.

In some embodiments, the TILs are not stored after the primary first expansion and prior to the rapid second expansion, and the TILs proceed directly to the rapid second expansion (for example, in some embodiments, there is no storage during the transition from Step B to Step D as shown in FIG. 8 (in particular, e.g., FIG. 8A and/or FIG. 8B and/or FIG. 8C and/or FIG. 8D)). In some embodiments, the transition occurs in closed system, as described herein. In some embodiments, the TILs from the priming first expansion, the second population of TILs, proceeds directly into the rapid second expansion with no transition period.

In some embodiments, the transition from the priming first expansion to the rapid second expansion, for example, Step C according to FIG. 8 (in particular, e.g., FIG. 8A and/or FIG. 8B and/or FIG. 8C and/or FIG. 8D), is performed in a closed system bioreactor. In some embodiments, a closed system is employed for the TIL expansion, as described herein. In some embodiments, a single bioreactor is employed. In some embodiments, the single bioreactor employed is for example a GREX-10 or a GREX-100. In some embodiments, the closed system bioreactor is a single bioreactor. In some embodiments, the transition from the priming first expansion to the rapid second expansion involves a scale-up in container size. In some embodiments, the priming first expansion is performed in a smaller container than the rapid second expansion. In some embodiments, the priming first expansion is performed in a GREX-100 and the rapid second expansion is performed in a GREX-500.

### D. STEP D: Rapid Second Expansion

In some embodiments, the TIL cell population is further expanded in number after harvest and the priming first expansion, after Step A and Step B, and the transition referred to as Step C, as indicated in FIG. 8 (in particular, e.g., FIG. 8A and/or FIG. 8B and/or FIG. 8C and/or FIG. 8D). This further expansion is referred to herein as the rapid second expansion or a rapid expansion, which can include expansion processes generally referred to in the art as a rapid expansion process (Rapid Expansion Protocol or REP; as well as processes as indicated in Step D of FIG. 8 (in particular, e.g., FIG. 8A and/or FIG. 8B and/or FIG. 8C and/or FIG. 8D). The rapid second expansion is generally accomplished using a culture media comprising a number of components, including feeder cells, a cytokine source, and an anti-CD3 antibody, in a gas-permeable container. In some embodiments, 1 day, 2 days, 3 days, or 4 days after initiation of the rapid second expansion (i.e., at days 8, 9, 10, or 11 of the overall Gen 3 process), the TILs are transferred to a larger volume container.

In some embodiments, the rapid second expansion (which can include expansions sometimes referred to as REP; as well as processes as indicated in Step D of FIG. 8 (in particular, e.g., FIG. 8A and/or FIG. 8B and/or FIG. 8C and/or FIG. 8D)) of TIL can be performed using any TIL flasks or containers known by those of skill in the art. In some embodiments, the second TIL expansion can proceed for 1 day, 2 days, 3 days, 4, days, 5 days, 6 days, 7 days, 8 days, 9 days or 10 days after initiation of the rapid second expansion. In some embodiments, the second TIL expansion can proceed for about 1 days to about 9 days after initiation of the rapid second expansion. In some embodiments, the second TIL expansion can proceed for about 1 days to about 10 days after initiation of the rapid second expansion. In some embodiments, the second TIL expansion can proceed for about 2 days to about 9 days after initiation of the rapid second expansion. In some embodiments, the second TIL expansion can proceed for about 2 days to about 10 days after initiation of the rapid second expansion. In some embodiments, the second TIL expansion can proceed for about 3 days to about 9 days after initiation of the rapid second expansion. In some embodiments, the second TIL expansion can proceed for about 3 days to about 10 days after initiation of the rapid second expansion. In some embodiments, the second TIL expansion can proceed for about 4 days to about 9 days after initiation of the rapid second expansion. In some embodiments, the second TIL expansion can proceed for about 4 days to about 10 days after initiation of the rapid second expansion. In some embodiments, the second TIL expansion can proceed for about 5 days to about 9 days after initiation of the rapid second expansion. In some embodiments, the second TIL expansion can proceed for about 5 days to about 10 days after initiation of the rapid second expansion. In some embodiments, the second TIL expansion can proceed for about 6 days to about 9 days after initiation of the rapid second expansion. In some embodiments, the second TIL expansion can proceed for about 6 days to about 10 days after initiation of the rapid second expansion. In some embodiments, the second TIL expansion can proceed for about 7 days to about 9 days after initiation of the rapid second expansion. In some embodiments, the second TIL expansion can proceed for about 7 days to about 10 days after initiation of the rapid second expansion. In some embodiments, the second TIL expansion can proceed for about 8 days to about 9 days after initiation of the rapid second expansion. In some embodiments, the second TIL expansion can proceed for about 8 days to about 10 days after initiation of the rapid second expansion. In some embodiments, the second TIL expansion can proceed for about 9 days to about 10 days after initiation of the rapid second expansion. In some embodiments, the second TIL expansion can proceed for about 1 day after initiation of the rapid second expansion. In some embodiments, the second TIL expansion can proceed for about 2 days after initiation of the rapid second expansion. In some embodiments, the second TIL expansion can proceed for about 3 days after initiation of the rapid second expansion. In some embodiments, the second TIL expansion can proceed for about 4 days after initiation of the rapid second expansion. In some embodiments, the second TIL expansion can proceed for about 5 days after initiation of the rapid second expansion. In some embodiments, the second TIL expansion can proceed for about 6 days after initiation of the rapid second expansion. In some embodiments, the second TIL expansion can proceed for about 7 days after initiation of the rapid second expansion. In some embodiments, the second TIL expansion can proceed for about 8 days after initiation of the rapid second expansion. In some embodiments, the second TIL expansion can proceed for about 9 days after initiation of the rapid second expansion. In some embodiments, the second TIL expansion can proceed for about 10 days after initiation of the rapid second expansion.

In some embodiments, the rapid second expansion can be performed in a gas permeable container using the methods of the present disclosure (including, for example, expansions referred to as REP; as well as processes as indicated in Step D of FIG. 8 (in particular, e.g., FIG. 8A and/or FIG. 8B and/or FIG. 8C and/or FIG. 8D). In some embodiments, the TILs are expanded in the rapid second expansion in the presence of IL-2, OKT-3, and feeder cells (also referred herein as “antigen-presenting cells”). In some embodiments, the TILs are expanded in the rapid second expansion in the presence of IL-2, OKT-3, and feeder cells, wherein the feeder cells are added to a final concentration that is twice, 2.4 times, 2.5 times, 3 times, 3.5 times or 4 times the concentration of feeder cells present in the priming first expansion. For example, TILs can be rapidly expanded using non-specific T-cell receptor stimulation in the presence of interleukin-2 (IL-2) or interleukin-15 (IL-15). The non-specific T-cell receptor stimulus can include, for example, an anti-CD3 antibody, such as about 30 ng/mL of OKT3, a mouse monoclonal anti-CD3 antibody (commercially available from Ortho-McNeil, Raritan, NJ or Miltenyi Biotech, Auburn, CA) or UHCT-1 (commercially available from BioLegend, San Diego, CA, USA). TILs can be expanded to induce further stimulation of the TILs in vitro by including one or more antigens during the second expansion, including antigenic portions thereof, such as epitope(s), of the cancer, which can be optionally expressed from a vector, such as a human leukocyte antigen A2 (HLA-A2) binding peptide, e.g., 0.3 μM MART-1:26-35 (27 L) or gpl 00:209-217 (210M), optionally in the presence of a T-cell growth factor, such as 300 IU/mL IL-2 or IL-15. Other suitable antigens may include, e.g., NY-ESO-1, TRP-1, TRP-2, tyrosinase cancer antigen, MAGE-A3, SSX-2, and VEGFR2, or antigenic portions thereof. TIL may also be rapidly expanded by re-stimulation with the same antigen(s) of the cancer pulsed onto HLA-A2-expressing antigen-presenting cells. Alternatively, the TILs can be further re-stimulated with, e.g., example, irradiated, autologous lymphocytes or with irradiated HLA-A2+ allogeneic lymphocytes and IL-2. In some embodiments, the re-stimulation occurs as part of the second expansion. In some embodiments, the second expansion occurs in the presence of irradiated, autologous lymphocytes or with irradiated HLA-A2+ allogeneic lymphocytes and IL-2.

In some embodiments, the cell culture medium further comprises IL-2. In some embodiments, the cell culture medium comprises about 3000 IU/mL of IL-2. In some embodiments, the cell culture medium comprises about 1000 IU/mL, about 1500 IU/mL, about 2000 IU/mL, about 2500 IU/mL, about 3000 IU/mL, about 3500 IU/mL, about 4000 IU/mL, about 4500 IU/mL, about 5000 IU/mL, about 5500 IU/mL, about 6000 IU/mL, about 6500 IU/mL, about 7000 IU/mL, about 7500 IU/mL, or about 8000 IU/mL of IL-2. In some embodiments, the cell culture medium comprises between 1000 and 2000 IU/mL, between 2000 and 3000 IU/mL, between 3000 and 4000 IU/mL, between 4000 and 5000 IU/mL, between 5000 and 6000 IU/mL, between 6000 and 7000 IU/mL, between 7000 and 8000 IU/mL, or between 8000 IU/mL of IL-2.

In some embodiments, the cell culture medium comprises OKT-3 antibody. In some embodiments, the cell culture medium comprises about 30 ng/mL of OKT-3 antibody. In some embodiments, the cell culture medium comprises about 0.1 ng/mL, about 0.5 ng/mL, about 1 ng/mL, about 2.5 ng/mL, about 5 ng/mL, about 7.5 ng/mL, about 10 ng/mL, about 15 ng/mL, about 20 ng/mL, about 25 ng/mL, about 30 ng/mL, about 35 ng/mL, about 40 ng/mL, about 50 ng/mL, about 60 ng/mL, about 70 ng/mL, about 80 ng/mL, about 90 ng/mL, about 100 ng/mL, about 200 ng/mL, about 500 ng/mL, and about 1 μg/mL of OKT-3 antibody. In some embodiments, the cell culture medium comprises between 0.1 ng/mL and 1 ng/mL, between 1 ng/mL and 5 ng/mL, between 5 ng/mL and 10 ng/mL, between 10 ng/mL and 20 ng/mL, between 20 ng/mL and 30 ng/mL, between 30 ng/mL and 40 ng/mL, between 40 ng/mL and 50 ng/mL, and between 50 ng/mL and 100 ng/mL of OKT-3 antibody. In some embodiments, the cell culture medium comprises between 15 ng/mL and 30 ng/mL of OKT-3 antibody. In some embodiments, the cell culture medium comprises between 30 ng/mL and 60 ng/mL of OKT-3 antibody. In some embodiments, the cell culture medium comprises about 30 ng/mL OKT-3. In some embodiments, the cell culture medium comprises about 60 ng/mL OKT-3. In some embodiments, the OKT-3 antibody is muromonab.

In some embodiments, the media in the rapid second expansion comprises IL-2. In some embodiments, the media comprises 6000 IU/mL of IL-2. In some embodiments, the media in the rapid second expansion comprises antigen-presenting feeder cells. In some embodiments, the media in the rapid second expansion comprises 7.5×10⁸ antigen-presenting feeder cells per container. In some embodiments, the media in the rapid second expansion comprises OKT-3. In some embodiments, the in the rapid second expansion media comprises 500 mL of culture medium and 30 μg of OKT-3 per container. In some embodiments, the container is a G-REX-100 MCS flask. In some embodiments, the in the rapid second expansion media comprises 6000 IU/mL of IL-2, 60 ng/mL of OKT-3, and 7.5×10⁸ antigen-presenting feeder cells. In some embodiments, the media comprises 500 mL of culture medium and 6000 IU/mL of IL-2, 30 μg of OKT-3, and 7.5×10⁸ antigen-presenting feeder cells per container.

In some embodiments, the media in the rapid second expansion comprises IL-2. In some embodiments, the media comprises 6000 IU/mL of IL-2. In some embodiments, the media in the rapid second expansion comprises antigen-presenting feeder cells. In some embodiments, the media comprises between 5×10⁸ and 7.5×10⁸ antigen-presenting feeder cells per container. In some embodiments, the media in the rapid second expansion comprises OKT-3. In some embodiments, the media in the rapid second expansion comprises 500 mL of culture medium and 30 μg of OKT-3 per container. In some embodiments, the container is a G-REX-100 MCS flask. In some embodiments, the media in the rapid second expansion comprises 6000 IU/mL of IL-2, 60 ng/mL of OKT-3, and between 5×10⁸ and 7.5×10⁸ antigen-presenting feeder cells. In some embodiments, the media in the rapid second expansion comprises 500 mL of culture medium and 6000 IU/mL of IL-2, 30 μg of OKT-3, and between 5×10⁸ and 7.5×10⁸ antigen-presenting feeder cells per container.

In some embodiments, the cell culture medium comprises one or more TNFRSF agonists in a cell culture medium. In some embodiments, the TNFRSF agonist comprises a 4-1BB agonist. In some embodiments, the TNFRSF agonist is a 4-1BB agonist, and the 4-1BB agonist is selected from the group consisting of urelumab, utomilumab, EU-101, a fusion protein, and fragments, derivatives, variants, biosimilars, and combinations thereof. In some embodiments, the TNFRSF agonist is added at a concentration sufficient to achieve a concentration in the cell culture medium of between 0.1 μg/mL and 100 μg/mL. In some embodiments, the TNFRSF agonist is added at a concentration sufficient to achieve a concentration in the cell culture medium of between 20 μg/mL and 40 μg/mL.

In some embodiments, in addition to one or more TNFRSF agonists, the cell culture medium further comprises IL-2 at an initial concentration of about 3000 IU/mL and OKT-3 antibody at an initial concentration of about 30 ng/mL, and wherein the one or more TNFRSF agonists comprises a 4-1BB agonist.

In some embodiments, a combination of IL-2, IL-7, IL-15, and/or IL-21 are employed as a combination during the second expansion. In some embodiments, IL-2, IL-7, IL-15, and/or IL-21 as well as any combinations thereof can be included during the second expansion, including, for example during a Step D processes according to FIG. 8 (in particular, e.g., FIG. 8A and/or FIG. 8B and/or FIG. 8C and/or FIG. 8D), as well as described herein. In some embodiments, a combination of IL-2, IL-15, and IL-21 are employed as a combination during the second expansion. In some embodiments, IL-2, IL-15, and IL-21 as well as any combinations thereof can be included during Step D processes according to FIG. 8 (in particular, e.g., FIG. 8A and/or FIG. 8B and/or FIG. 8C and/or FIG. 8D) and as described herein.

In some embodiments, the second expansion can be conducted in a supplemented cell culture medium comprising IL-2, OKT-3, antigen-presenting feeder cells, and optionally a TNFRSF agonist. In some embodiments, the second expansion occurs in a supplemented cell culture medium. In some embodiments, the supplemented cell culture medium comprises IL-2, OKT-3, and antigen-presenting feeder cells. In some embodiments, the second cell culture medium comprises IL-2, OKT-3, and antigen-presenting cells (APCs; also referred to as antigen-presenting feeder cells). In some embodiments, the second expansion occurs in a cell culture medium comprising IL-2, OKT-3, and antigen-presenting feeder cells (i.e., antigen presenting cells).

In some embodiments, the second expansion culture media comprises about 500 IU/mL of IL-15, about 400 IU/mL of IL-15, about 300 IU/mL of IL-15, about 200 IU/mL of IL-15, about 180 IU/mL of IL-15, about 160 IU/mL of IL-15, about 140 IU/mL of IL-15, about 120 IU/mL of IL-15, or about 100 IU/mL of IL-15. In some embodiments, the second expansion culture media comprises about 500 IU/mL of IL-15 to about 100 IU/mL of IL-15. In some embodiments, the second expansion culture media comprises about 400 IU/mL of IL-15 to about 100 IU/mL of IL-15. In some embodiments, the second expansion culture media comprises about 300 IU/mL of IL-15 to about 100 IU/mL of IL-15. In some embodiments, the second expansion culture media comprises about 200 IU/mL of IL-15. In some embodiments, the cell culture medium comprises about 180 IU/mL of IL-15. In some embodiments, the cell culture medium further comprises IL-15. In some embodiments, the cell culture medium comprises about 180 IU/mL of IL-15.

In some embodiments, the second expansion culture media comprises about 20 IU/mL of IL-21, about 15 IU/mL of IL-21, about 12 IU/mL of IL-21, about 10 IU/mL of IL-21, about 5 IU/mL of IL-21, about 4 IU/mL of IL-21, about 3 IU/mL of IL-21, about 2 IU/mL of IL-21, about 1 IU/mL of IL-21, or about 0.5 IU/mL of IL-21. In some embodiments, the second expansion culture media comprises about 20 IU/mL of IL-21 to about 0.5 IU/mL of IL-21. In some embodiments, the second expansion culture media comprises about 15 IU/mL of IL-21 to about 0.5 IU/mL of IL-21. In some embodiments, the second expansion culture media comprises about 12 IU/mL of IL-21 to about 0.5 IU/mL of IL-21. In some embodiments, the second expansion culture media comprises about 10 IU/mL of IL-21 to about 0.5 IU/mL of IL-21. In some embodiments, the second expansion culture media comprises about 5 IU/mL of IL-21 to about 1 IU/mL of IL-21. In some embodiments, the second expansion culture media comprises about 2 IU/mL of IL-21. In some embodiments, the cell culture medium comprises about 1 IU/mL of IL-21. In some embodiments, the cell culture medium comprises about 0.5 IU/mL of IL-21. In some embodiments, the cell culture medium further comprises IL-21. In some embodiments, the cell culture medium comprises about 1 IU/mL of IL-21.

In some embodiments, the antigen-presenting feeder cells (APCs) are PBMCs. In some embodiments, the ratio of TILs to PBMCs and/or antigen-presenting cells in the rapid expansion and/or the second expansion is about 1 to 10, about 1 to 15, about 1 to 20, about 1 to 25, about 1 to 30, about 1 to 35, about 1 to 40, about 1 to 45, about 1 to 50, about 1 to 75, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to PBMCs in the rapid expansion and/or the second expansion is between 1 to 50 and 1 to 300. In some embodiments, the ratio of TILs to PBMCs in the rapid expansion and/or the second expansion is between 1 to 100 and 1 to 200.

In some embodiments, REP and/or the rapid second expansion is performed in flasks with the bulk TILs being mixed with a 100- or 200-fold excess of inactivated feeder cells, wherein the feeder cell concentration is at least 1.1 times (1.1×), 1.2×, 1.3×, 1.4×, 1.5×, 1.6×, 1.7×, 1.8×, 1.8×, 2×, 2.1×2.2×, 2.3×, 2.4×, 2.5×, 2.6×, 2.7×, 2.8×, 2.9×, 3.0×, 3.1×, 3.2×, 3.3×, 3.4×, 3.5×, 3.6×, 3.7×, 3.8×, 3.9× or 4.0× the feeder cell concentration in the priming first expansion, 30 ng/mL OKT3 anti-CD3 antibody and 6000 IU/mL IL-2 in 150 mL media. Media replacement is done (generally 2/3 media replacement via aspiration of 2/3 of spent media and replacement with an equal volume of fresh media) until the cells are transferred to an alternative growth chamber. Alternative growth chambers include G-REX flasks and gas permeable containers as more fully discussed below.

In some embodiments, the rapid second expansion (which can include processes referred to as the REP process) is 7 to 9 days, as discussed in the examples and figures. In some embodiments, the second expansion is 7 days. In some embodiments, the second expansion is 8 days. In some embodiments, the second expansion is 9 days.

In some embodiments, the second expansion (which can include expansions referred to as REP, as well as those referred to in Step D of FIG. 8 (in particular, e.g., FIG. 8A and/or FIG. 8B and/or FIG. 8C and/or FIG. 8D) may be performed in 500 mL capacity gas permeable flasks with 100 cm gas-permeable silicon bottoms (G-REX-100, commercially available from Wilson Wolf Manufacturing Corporation, New Brighton, MN, USA), 5×10⁶ or 10×10⁶ TIL may be cultured with PBMCs in 400 mL of 50/50 medium, supplemented with 5% human AB serum, 3000 IU per mL of IL-2 and 30 ng per mL of anti-CD3 (OKT3). The G-REX-100 flasks may be incubated at 37° C. in 5% CO₂. On day 5, 250 mL of supernatant may be removed and placed into centrifuge bottles and centrifuged at 1500 rpm (491×g) for 10 minutes. The TIL pellets may be re-suspended with 150 mL of fresh medium with 5% human AB serum, 6000 IU per mL of IL-2, and added back to the original GREX-100 flasks. When TIL are expanded serially in GREX-100 flasks, on day 10 or 11 the TILs can be moved to a larger flask, such as a GREX-500. The cells may be harvested on day 14 of culture. The cells may be harvested on day 15 of culture. The cells may be harvested on day 16 of culture. In some embodiments, media replacement is done until the cells are transferred to an alternative growth chamber. In some embodiments, 2/3 of the media is replaced by aspiration of spent media and replacement with an equal volume of fresh media. In some embodiments, alternative growth chambers include GREX flasks and gas permeable containers as more fully discussed below.

In some embodiments, the culture medium used in the expansion processes disclosed herein is a serum-free medium or a defined medium. In some embodiments, the serum-free or defined medium comprises a basal cell medium and a serum supplement and/or a serum replacement. In some embodiments, the serum-free or defined medium is used to prevent and/or decrease experimental variation due in part to the lot-to-lot variation of serum-containing media.

In some embodiments, the serum-free or defined medium comprises a basal cell medium and a serum supplement and/or serum replacement. In some embodiments, the basal cell medium includes, but is not limited to CTS™ OpTmizer™ T-cell Expansion Basal Medium, CTS™ OpTmizer™ T-Cell Expansion SFM, CTS™ AIM-V Medium, CTS™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (uMEM), Glasgow's Minimal Essential Medium (G-MEM), RPMI growth medium, and Iscove's Modified Dulbecco's Medium.

In some embodiments, the serum supplement or serum replacement includes, but is not limited to one or more of CTS™ OpTmizer T-Cell Expansion Serum Supplement, CTS™ Immune Cell Serum Replacement, one or more albumins or albumin substitutes, one or more amino acids, one or more vitamins, one or more transferrins or transferrin substitutes, one or more antioxidants, one or more insulins or insulin substitutes, one or more collagen precursors, one or more antibiotics, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn²⁺ and Zr⁴⁺. In some embodiments, the defined medium further comprises L-glutamine, sodium bicarbonate and/or 2-mercaptoethanol.

In some embodiments, the CTS™OpTmizer™ T-cell Immune Cell Serum Replacement is used with conventional growth media, including but not limited to CTS™ OpTmizer™ T-cell Expansion Basal Medium, CTS™ OpTmizer™ T-cell Expansion SFM, CTS™ AIM-V Medium, CST™ AIM-V SFM, LymphoONE™ T-Cell Expansion Xeno-Free Medium, Dulbecco's Modified Eagle's Medium (DMEM), Minimal Essential Medium (MEM), Basal Medium Eagle (BME), RPMI 1640, F-10, F-12, Minimal Essential Medium (uMEM), Glasgow's Minimal Essential Medium (G-MEM), RPMI growth medium, and Iscove's Modified Dulbecco's Medium.

In some embodiments, the total serum replacement concentration (vol %) in the serum-free or defined medium is from about 1%, 2%, 3%, 4%, 5%, 6%, 7%, 8%, 9%, 10%, 11%, 12%, 13%, 14%, 15%, 16%, 17%, 18%, 19%, or 20% by volume of the total serum-free or defined medium. In some embodiments, the total serum replacement concentration is about 3% of the total volume of the serum-free or defined medium. In some embodiments, the total serum replacement concentration is about 5% of the total volume of the serum-free or defined medium. In some embodiments, the total serum replacement concentration is about 10% of the total volume of the serum-free or defined medium.

In some embodiments, the serum-free or defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM.

In some embodiments, the defined medium is CTS™ OpTmizer™ T-cell Expansion SFM (ThermoFisher Scientific). Any formulation of CTS™ OpTmizer™ is useful in the present invention. CTS™ OpTmizer™ T-cell Expansion SFM is a combination of 1 L CTS™ OpTmizer™ T-cell Expansion Basal Medium and 26 mL CTS™ OpTmizer™ T-Cell Expansion Supplement, which are mixed together prior to use. In some embodiments, the CTS™ OpTmizer™ T-cell Expansion SFM is supplemented with about 3% of the CTS™ Immune Cell Serum Replacement (SR) (ThermoFisher Scientific), along with 2-mercaptoethanol at 55 mM. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific), 55 mM of 2-mercaptoethanol, and 2 mM of L-glutamine, and further comprises about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and 55 mM of 2-mercaptoethanol, and further comprises about 1000 IU/mL to about 6000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 1000 IU/mL to about 8000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 3000 IU/mL of IL-2. In some embodiments, the CTS™OpTmizer™ T-cell Expansion SFM is supplemented with about 3% of the CTS™ Immune Cell Serum Replacement (SR) (ThermoFisher Scientific) and about 2 mM glutamine, and further comprises about 6000 IU/mL of IL-2.

In some embodiments, the serum-free medium or defined medium is supplemented with glutamine (i.e., GlutaMAX®) at a concentration of from about 0.1 mM to about 10 mM, 0.5 mM to about 9 mM, 1 mM to about 8 mM, 2 mM to about 7 mM, 3 mM to about 6 mM, or 4 mM to about 5 mM. In some embodiments, the serum-free medium or defined medium is supplemented with glutamine (i.e., GlutaMAX®) at a concentration of about 2 mM.

In some embodiments, the serum-free medium or defined medium is supplemented with 2-mercaptoethanol at a concentration of from about 5 mM to about 150 mM, 10 mM to about 140 mM, 15 mM to about 130 mM, 20 mM to about 120 mM, 25 mM to about 110 mM, 30 mM to about 100 mM, 35 mM to about 95 mM, 40 mM to about 90 mM, 45 mM to about 85 mM, 50 mM to about 80 mM, 55 mM to about 75 mM, 60 mM to about 70 mM, or about 65 mM. In some embodiments, the serum-free medium or defined medium is supplemented with 2-mercaptoethanol at a concentration of about 55 mM.

In some embodiments, the defined media described in International Patent Application Publication No. WO1998/030679 and U.S. Patent Application Publication No. US 2002/0076747 A1, which is herein incorporated by reference, are useful in the present invention. In that publication, serum-free eukaryotic cell culture media are described. The serum-free, eukaryotic cell culture medium includes a basal cell culture medium supplemented with a serum-free supplement capable of supporting the growth of cells in serum-free culture. The serum-free eukaryotic cell culture medium supplement comprises or is obtained by combining one or more ingredients selected from the group consisting of one or more albumins or albumin substitutes, one or more amino acids, one or more vitamins, one or more transferrins or transferrin substitutes, one or more antioxidants, one or more insulins or insulin substitutes, one or more collagen precursors, one or more trace elements, and one or more antibiotics. In some embodiments, the defined medium further comprises L-glutamine, sodium bicarbonate and/or beta-mercaptoethanol. In some embodiments, the defined medium comprises an albumin or an albumin substitute and one or more ingredients selected from group consisting of one or more amino acids, one or more vitamins, one or more transferrins or transferrin substitutes, one or more antioxidants, one or more insulins or insulin substitutes, one or more collagen precursors, and one or more trace elements. In some embodiments, the defined medium comprises albumin and one or more ingredients selected from the group consisting of glycine, L-histidine, L-isoleucine, L-methionine, L-phenylalanine, L-proline, L-hydroxyproline, L-serine, L-threonine, L-tryptophan, L-tyrosine, L-valine, thiamine, reduced glutathione, L-ascorbic acid-2-phosphate, iron saturated transferrin, insulin, and compounds containing the trace element moieties Ag⁺, Al³⁺, Ba²⁺, Cd²⁺, Co²⁺, Cr³⁺, Ge⁴⁺, Se⁴⁺, Br, T, Mn²⁺, P, Si⁴⁺, V⁵⁺, Mo⁶⁺, Ni²⁺, Rb⁺, Sn⁺ and Zr⁴⁺. In some embodiments, the basal cell media is selected from the group consisting of Dulbecco's Modified Eagle's Medium (DMEM), Minimal Essential Medium (MEM), Basal Medium Eagle (BME), RPMI 1640, F-10, F-12, Minimal Essential Medium (αMEM), Glasgow's Minimal Essential Medium (G-MEM), RPMI growth medium, and Iscove's Modified Dulbecco's Medium.

In some embodiments, the concentration of glycine in the defined medium is in the range of from about 5-200 mg/L, the concentration of L-histidine is about 5-250 mg/L, the concentration of L-isoleucine is about 5-300 mg/L, the concentration of L-methionine is about 5-200 mg/L, the concentration of L-phenylalanine is about 5-400 mg/L, the concentration of L-proline is about 1-1000 mg/L, the concentration of L-hydroxyproline is about 1-45 mg/L, the concentration of L-serine is about 1-250 mg/L, the concentration of L-threonine is about 10-500 mg/L, the concentration of L-tryptophan is about 2-110 mg/L, the concentration of L-tyrosine is about 3-175 mg/L, the concentration of L-valine is about 5-500 mg/L, the concentration of thiamine is about 1-20 mg/L, the concentration of reduced glutathione is about 1-20 mg/L, the concentration of L-ascorbic acid-2-phosphate is about 1-200 mg/L, the concentration of iron saturated transferrin is about 1-50 mg/L, the concentration of insulin is about 1-100 mg/L, the concentration of sodium selenite is about 0.000001-0.0001 mg/L, and the concentration of albumin (e.g., AlbuMAX® I) is about 5000-50,000 mg/L.

In some embodiments, the non-trace element moiety ingredients in the defined medium are present in the concentration ranges listed in the column under the heading “Concentration Range in 1× Medium” in Table 12. In other embodiments, the non-trace element moiety ingredients in the defined medium are present in the final concentrations listed in the column under the heading “A Preferred Embodiment of the 1× Medium” in Table 12. In other embodiments, the defined medium is a basal cell medium comprising a serum free supplement. In some of these embodiments, the serum free supplement comprises non-trace moiety ingredients of the type and in the concentrations listed in the column under the heading “A Preferred Embodiment in Supplement” in Table 12.

In some embodiments, the osmolarity of the defined medium is between about 260 and 350 mOsmol. In some embodiments, the osmolarity is between about 280 and 310 mOsmol. In some embodiments, the defined medium is supplemented with up to about 3.7 g/L, or about 2.2 g/L sodium bicarbonate. The defined medium can be further supplemented with L-glutamine (final concentration of about 2 mM), one or more antibiotics, non-essential amino acids (NEAA; final concentration of about 100 μM), 2-mercaptoethanol (final concentration of about 100 μM).

In some embodiments, the defined media described in Smith, et al., Clin. Transl. Immunology, 4(1), 2015 (doi: 10.1038/cti.2014.31) are useful in the present invention. Briefly, RPMI or CTS™ OpTmizer™ was used as the basal cell medium, and supplemented with either 0, 2%, 5%, or 10% CTS™ Immune Cell Serum Replacement.

In some embodiments, the cell medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME or PME; also known as 2-mercaptoethanol, CAS 60-24-2).

In some embodiments, the rapid second expansion (including expansions referred to as REP) is performed and further comprises a step wherein TILs are selected for superior tumor reactivity. Any selection method known in the art may be used. For example, the methods described in U.S. Patent Application Publication No. 2016/0010058 A1, the disclosures of which are incorporated herein by reference, may be used for selection of TILs for superior tumor reactivity.

Optionally, a cell viability assay can be performed after the rapid second expansion (including expansions referred to as the REP expansion), using standard assays known in the art. For example, a trypan blue exclusion assay can be done on a sample of the bulk TILs, which selectively labels dead cells and allows a viability assessment. In some embodiments, TIL samples can be counted and viability determined using a Cellometer K2 automated cell counter (Nexcelom Bioscience, Lawrence, MA). In some embodiments, viability is determined according to the standard Cellometer K2 Image Cytometer Automatic Cell Counter protocol.

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained in the second expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRα/β).

In some embodiments, the rapid second expansion culture medium (e.g., sometimes referred to as CM2 or the second cell culture medium), comprises IL-2, OKT-3, as well as the antigen-presenting feeder cells (APCs), as discussed in more detail below. In some embodiments, the rapid second expansion culture medium (e.g., sometimes referred to as CM2 or the second cell culture medium), comprises 6000 IU/mL IL-2, 30 ug/flask OKT-3, as well as 7.5×10⁸ antigen-presenting feeder cells (APCs), as discussed in more detail below. In some embodiments, the rapid second expansion culture medium (e.g., sometimes referred to as CM2 or the second cell culture medium), comprises IL-2, OKT-3, as well as the antigen-presenting feeder cells (APCs), as discussed in more detail below. In some embodiments, the rapid second expansion culture medium (e.g., sometimes referred to as CM2 or the second cell culture medium), comprises 6000 IU/mL IL-2, 30 ug/flask OKT-3, as well as 5×10⁸ antigen-presenting feeder cells (APCs), as discussed in more detail below.

In some embodiments, the rapid second expansion, for example, Step D according to FIG. 8 (in particular, e.g., FIG. 8A and/or FIG. 8B and/or FIG. 8C and/or FIG. 8D), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-100 or a G-REX-500. In some embodiments, the bioreactor employed is a G-REX-100. In some embodiments, the bioreactor employed is a G-REX-500.

In some embodiments, the step of rapid second expansion is split into a plurality of steps to achieve a scaling up of the culture by: (a) performing the rapid second expansion by culturing TILs in a small scale culture in a first container, e.g., a G-REX-100 MCS container, for a period of about 3 to 7 days, and then (b) effecting the transfer of the TILs in the small scale culture to a second container larger than the first container, e.g., a G-REX-500-MCS container, and culturing the TILs from the small scale culture in a larger scale culture in the second container for a period of about 4 to 7 days.

In some embodiments, the step of rapid second expansion is split into a plurality of steps to achieve a scaling out of the culture by: (a) performing the rapid second expansion by culturing TILs in a first small scale culture in a first container, e.g., a G-REX-100 MCS container, for a period of about 3 to 7 days, and then (b) effecting the transfer and apportioning of the TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the TILs from first small scale culture transferred to such second container is cultured in a second small scale culture for a period of about 4 to 7 days.

In some embodiments, the first small scale TIL culture is apportioned into a plurality of about 2 to 5 subpopulations of TILs.

In some embodiments, the step of rapid second expansion is split into a plurality of steps to achieve a scaling out and scaling up of the culture by: (a) performing the rapid second expansion by culturing TILs in a small scale culture in a first container, e.g., a G-REX-100 MCS container, for a period of about 3 to 7 days, and then (b) effecting the transfer and apportioning of the TILs from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX-500MCS containers, wherein in each second container the portion of the TILs from the small scale culture transferred to such second container is cultured in a larger scale culture for a period of about 4 to 7 days.

In some embodiments, the step of rapid second expansion is split into a plurality of steps to achieve a scaling out and scaling up of the culture by: (a) performing the rapid or second expansion by culturing TILs in a small scale culture in a first container, e.g., a G-REX-100 MCS container, for a period of about 5 days, and then (b) effecting the transfer and apportioning of the TILs from the small scale culture into and amongst 2, 3 or 4 second containers that are larger in size than the first container, e.g., G-REX-500 MCS containers, wherein in each second container the portion of the TILs from the small scale culture transferred to such second container is cultured in a larger scale culture for a period of about 6 days.

In some embodiments, upon the splitting of the rapid second expansion, each second container comprises at least 10⁸ TILs. In some embodiments, upon the splitting of the rapid or second expansion, each second container comprises at least 10⁸ TILs, at least 10⁹ TILs, or at least 10¹⁰ TILs. In one exemplary embodiment, each second container comprises at least 10¹⁰ TILs.

In some embodiments, the first small scale TIL culture is apportioned into a plurality of subpopulations. In some embodiments, the first small scale TIL culture is apportioned into a plurality of about 2 to 5 subpopulations. In some embodiments, the first small scale TIL culture is apportioned into a plurality of about 2, 3, 4, or 5 subpopulations.

In some embodiments, after the completion of the rapid second expansion, the plurality of subpopulations comprises a therapeutically effective amount of TILs. In some embodiments, after the completion of the rapid or second expansion, one or more subpopulations of TILs are pooled together to produce a therapeutically effective amount of TILs. In some embodiments, after the completion of the rapid expansion, each subpopulation of TILs comprises a therapeutically effective amount of TILs.

In some embodiments, the rapid second expansion is performed for a period of about 3 to 7 days before being split into a plurality of steps. In some embodiments, the splitting of the rapid second expansion occurs at about day 3, day 4, day 5, day 6, or day 7 after the initiation of the rapid or second expansion.

In some embodiments, the splitting of the rapid second expansion occurs at about day 7, day 8, day 9, day 10, day 11, day 12, day 13, day 14, day 15, or day 16 day 17, or day 18 after the initiation of the first expansion (i.e., pre-REP expansion). In one exemplary embodiment, the splitting of the rapid or second expansion occurs at about day 16 after the initiation of the first expansion.

In some embodiments, the rapid second expansion is further performed for a period of about 7 to 11 days after the splitting. In some embodiments, the rapid second expansion is further performed for a period of about 5 days, 6 days, 7 days, 8 days, 9 days, 10 days, or 11 days after the splitting.

In some embodiments, the cell culture medium used for the rapid second expansion before the splitting comprises the same components as the cell culture medium used for the rapid second expansion after the splitting. In some embodiments, the cell culture medium used for the rapid second expansion before the splitting comprises different components from the cell culture medium used for the rapid second expansion after the splitting.

In some embodiments, the cell culture medium used for the rapid second expansion before the splitting comprises IL-2, optionally OKT-3 and further optionally APCs. In some embodiments, the cell culture medium used for the rapid second expansion before the splitting comprises IL-2, OKT-3, and further optionally APCs. In some embodiments, the cell culture medium used for the rapid second expansion before the splitting comprises IL-2, OKT-3 and APCs.

In some embodiments, the cell culture medium used for the rapid second expansion before the splitting is generated by supplementing the cell culture medium in the first expansion with fresh culture medium comprising IL-2, optionally OKT-3 and further optionally APCs. In some embodiments, the cell culture medium used for the rapid second expansion before the splitting is generated by supplementing the cell culture medium in the first expansion with fresh culture medium comprising IL-2, OKT-3 and APCs. In some embodiments, the cell culture medium used for the rapid second expansion before the splitting is generated by replacing the cell culture medium in the first expansion with fresh cell culture medium comprising IL-2, optionally OKT-3 and further optionally APCs. In some embodiments, the cell culture medium used for the rapid second expansion before the splitting is generated by replacing the cell culture medium in the first expansion with fresh cell culture medium comprising IL-2, OKT-3 and APCs.

In some embodiments, the cell culture medium used for the rapid second expansion after the splitting comprises IL-2, and optionally OKT-3. In some embodiments, the cell culture medium used for the rapid second expansion after the splitting comprises IL-2, and OKT-3. In some embodiments, the cell culture medium used for the rapid second expansion after the splitting is generated by replacing the cell culture medium used for the rapid second expansion before the splitting with fresh culture medium comprising IL-2 and optionally OKT-3. In some embodiments, the cell culture medium used for the rapid second expansion after the splitting is generated by replacing the cell culture medium used for the rapid second expansion before the splitting with fresh culture medium comprising IL-2 and OKT-3.

### 1. Feeder Cells and Antigen Presenting Cells

In some embodiments, the rapid second expansion procedures described herein (for example including expansion such as those described in Step D from FIG. 8 (in particular, e.g., FIG. 8A and/or FIG. 8B and/or FIG. 8C and/or FIG. 8D), as well as those referred to as REP) require an excess of feeder cells during REP TIL expansion and/or during the rapid second expansion. In many embodiments, the feeder cells are peripheral blood mononuclear cells (PBMCs) obtained from standard whole blood units from healthy blood donors. The PBMCs are obtained using standard methods such as Ficoll-Paque gradient separation.

In general, the allogeneic PBMCs are inactivated, either via irradiation or heat treatment, and used in the REP procedures, as described in the examples, which provides an exemplary protocol for evaluating the replication incompetence of irradiate allogeneic PBMCs.

In some embodiments, PBMCs are considered replication incompetent and acceptable for use in the TIL expansion procedures described herein if the total number of viable cells on day 7 or 14 is less than the initial viable cell number put into culture on day 0 of the REP and/or day 0 of the second expansion (i.e., the start day of the second expansion).

In some embodiments, PBMCs are considered replication incompetent and acceptable for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30 ng/mL OKT3 antibody and 3000 IU/mL IL-2. In some embodiments, the PBMCs are cultured in the presence of 60 ng/mL OKT3 antibody and 6000 IU/mL IL-2. In some embodiments, the PBMCs are cultured in the presence of 60 ng/mL OKT3 antibody and 3000 IU/mL IL-2. In some embodiments, the PBMCs are cultured in the presence of 30 ng/mL OKT3 antibody and 6000 IU/mL IL-2.

In some embodiments, PBMCs are considered replication incompetent and acceptable for use in the TIL expansion procedures described herein if the total number of viable cells, cultured in the presence of OKT3 and IL-2, on day 7 and day 14 has not increased from the initial viable cell number put into culture on day 0 of the REP and/or day 0 of the second expansion (i.e., the start day of the second expansion). In some embodiments, the PBMCs are cultured in the presence of 30-60 ng/mL OKT3 antibody and 1000-6000 IU/mL IL-2. In some embodiments, the PBMCs are cultured in the presence of 30-60 ng/mL OKT3 antibody and 2000-5000 IU/mL IL-2. In some embodiments, the PBMCs are cultured in the presence of 30-60 ng/mL OKT3 antibody and 2000-4000 IU/mL IL-2. In some embodiments, the PBMCs are cultured in the presence of 30-60 ng/mL OKT3 antibody and 2500-3500 IU/mL IL-2. In some embodiments, the PBMCs are cultured in the presence of 30-60 ng/mL OKT3 antibody and 6000 IU/mL IL-2.

In some embodiments, the antigen-presenting feeder cells are PBMCs. In some embodiments, the antigen-presenting feeder cells are artificial antigen-presenting feeder cells. In some embodiments, the ratio of TILs to antigen-presenting feeder cells in the second expansion is about 1 to 10, about 1 to 25, about 1 to 50, about 1 to 100, about 1 to 125, about 1 to 150, about 1 to 175, about 1 to 200, about 1 to 225, about 1 to 250, about 1 to 275, about 1 to 300, about 1 to 325, about 1 to 350, about 1 to 375, about 1 to 400, or about 1 to 500. In some embodiments, the ratio of TILs to antigen-presenting feeder cells in the second expansion is between 1 to 50 and 1 to 300. In some embodiments, the ratio of TILs to antigen-presenting feeder cells in the second expansion is between 1 to 100 and 1 to 200.

In some embodiments, the second expansion procedures described herein require a ratio of about 5×10⁸ feeder cells to about 100×10⁶ TILs. In some embodiments, the second expansion procedures described herein require a ratio of about 7.5×10⁸ feeder cells to about 100×10⁶ TILs. In other embodiments, the second expansion procedures described herein require a ratio of about 5×10⁸ feeder cells to about 50×10⁶ TILs. In other embodiments, the second expansion procedures described herein require a ratio of about 7.5×10⁸ feeder cells to about 50×10⁶ TILs. In yet other embodiments, the second expansion procedures described herein require about 5×10⁸ feeder cells to about 25×10⁶ TILs. In yet other embodiments, the second expansion procedures described herein require about 7.5×10⁸ feeder cells to about 25×10⁶ TILs. In yet other embodiments, the rapid second expansion requires twice the number of feeder cells as the rapid second expansion. In yet other embodiments, when the priming first expansion described herein requires about 2.5×10⁸ feeder cells, the rapid second expansion requires about 5×10⁸ feeder cells. In yet other embodiments, when the priming first expansion described herein requires about 2.5×10⁸ feeder cells, the rapid second expansion requires about 7.5×10⁸ feeder cells. In yet other embodiments, the rapid second expansion requires two times (2.0×), 2.5×, 3.0×, 3.5× or 4.0× the number of feeder cells as the priming first expansion.

In some embodiments, the rapid second expansion procedures described herein require an excess of feeder cells during the rapid second expansion. In many embodiments, the feeder cells are peripheral blood mononuclear cells (PBMCs) obtained from standard whole blood units from allogeneic healthy blood donors. The PBMCs are obtained using standard methods such as Ficoll-Paque gradient separation. In some embodiments, artificial antigen-presenting (aAPC) cells are used in place of PBMCs. In some embodiments, the PBMCs are added to the rapid second expansion at twice the concentration of PBMCs that were added to the priming first expansion.

In general, the allogeneic PBMCs are inactivated, either via irradiation or heat treatment, and used in the TIL expansion procedures described herein, including the exemplary procedures described in the figures and examples.

In some embodiments, artificial antigen presenting cells are used in the rapid second expansion as a replacement for, or in combination with, PBMCs.

### 2. Cytokines and Other Additives

The rapid second expansion methods described herein generally use culture media with high doses of a cytokine, in particular IL-2, as is known in the art.

Alternatively, using combinations of cytokines for the rapid second expansion of TILs is additionally possible, with combinations of two or more of IL-2, IL-15 and IL-21 as is described in U.S. Patent Application Publication No. US 2017/0107490 A1, the disclosure of which is incorporated by reference herein. Thus, possible combinations include IL-2 and IL-15, IL-2 and IL-21, IL-15 and IL-21, and IL-2, IL-15 and IL-21, with the latter finding particular use in many embodiments. The use of combinations of cytokines specifically favors the generation of lymphocytes, and in particular T-cells as described therein.

In some embodiments, Step D (from in particular, e.g., FIG. 8A and/or FIG. 8B and/or FIG. 8C and/or FIG. 8D) may also include the addition of OKT-3 antibody or muromonab to the culture media, as described elsewhere herein. In some embodiments, Step D may also include the addition of a 4-1BB agonist to the culture media, as described elsewhere herein. In some embodiments, Step D (from, in particular, e.g., FIG. 8A and/or FIG. 8B and/or FIG. 8C and/or FIG. 8D) may also include the addition of an OX-40 agonist to the culture media, as described elsewhere herein. In addition, additives such as peroxisome proliferator-activated receptor gamma coactivator I-alpha agonists, including proliferator-activated receptor (PPAR)-gamma agonists such as a thiazolidinedione compound, may be used in the culture media during Step D (from, in particular, e.g., FIG. 8A and/or FIG. 8B and/or FIG. 8C and/or FIG. 8D), as described in U.S. Patent Application Publication No. US 2019/0307796 A1, the disclosure of which is incorporated by reference herein.

### E. STEP E: Harvest TILs

After the rapid second expansion step, cells can be harvested. In some embodiments the TILs are harvested after one, two, three, four or more expansion steps, for example as provided in FIG. 8 (in particular, e.g., FIG. 8A and/or FIG. 8B and/or FIG. 8C and/or FIG. 8D). In some embodiments the TILs are harvested after two expansion steps, for example as provided in FIG. 8 (in particular, e.g., FIG. 8A and/or FIG. 8B and/or FIG. 8C and/or FIG. 8D). In some embodiments the TILs are harvested after two expansion steps, one priming first expansion and one rapid second expansion, for example as provided in FIG. 8 (in particular, e.g., FIG. 8A and/or FIG. 8B and/or FIG. 8C and/or FIG. 8D).

TILs can be harvested in any appropriate and sterile manner, including, for example by centrifugation. Methods for TIL harvesting are well known in the art and any such known methods can be employed with the present process. In some embodiments, TILs are harvested using an automated system.

Cell harvesters and/or cell processing systems are commercially available from a variety of sources, including, for example, Fresenius Kabi, Tomtec Life Science, Perkin Elmer, and Inotech Biosystems International, Inc. Any cell-based harvester can be employed with the present methods. In some embodiments, the cell harvester and/or cell processing system is a membrane-based cell harvester. In some embodiments, cell harvesting is via a cell processing system, such as the LOVO system (manufactured by Fresenius Kabi). The term “LOVO cell processing system” also refers to any instrument or device manufactured by any vendor that can pump a solution comprising cells through a membrane or filter such as a spinning membrane or spinning filter in a sterile and/or closed system environment, allowing for continuous flow and cell processing to remove supernatant or cell culture media without pelletization. In some embodiments, the cell harvester and/or cell processing system can perform cell separation, washing, fluid-exchange, concentration, and/or other cell processing steps in a closed, sterile system.

In some embodiments, the rapid second expansion, for example, Step D according to FIG. 8 (in particular, e.g., FIG. 8A and/or FIG. 8B and/or FIG. 8C and/or FIG. 8D), is performed in a closed system bioreactor. In some embodiments, a closed system is employed for the TIL expansion, as described herein. In some embodiments, a bioreactor is employed. In some embodiments, a bioreactor is employed as the container. In some embodiments, the bioreactor employed is for example a G-REX-100 or a G-REX-500. In some embodiments, the bioreactor employed is a G-REX-100. In some embodiments, the bioreactor employed is a G-REX-500.

In some embodiments, Step E according to FIG. 8 (in particular, e.g., FIG. 8A and/or FIG. 8B and/or FIG. 8C and/or FIG. 8D), is performed according to the processes described herein. In some embodiments, the closed system is accessed via syringes under sterile conditions in order to maintain the sterility and closed nature of the system. In some embodiments, a closed system as described herein is employed.

In some embodiments, TILs are harvested according to the methods described herein. In some embodiments, TILs between days 14 and 16 are harvested using the methods as described herein. In some embodiments, TILs are harvested at 14 days using the methods as described herein. In some embodiments, TILs are harvested at 15 days using the methods as described herein. In some embodiments, TILs are harvested at 16 days using the methods as described herein.

### F. STEP F: Final Formulation and Transfer to Infusion Container

After Steps A through E as provided in an exemplary order in FIG. 8 (in particular, e.g., FIG. 8A and/or FIG. 8B and/or FIG. 8C and/or FIG. 8D) and as outlined in detail above and herein are complete, cells are transferred to a container for use in administration to a patient, such as an infusion bag or sterile vial. In some embodiments, once a therapeutically sufficient number of TILs are obtained using the expansion methods described above, they are transferred to a container for use in administration to a patient.

In some embodiments, TILs expanded using the methods of the present disclosure are administered to a patient as a pharmaceutical composition. In some embodiments, the pharmaceutical composition is a suspension of TILs in a sterile buffer. TILs expanded as disclosed herein may be administered by any suitable route as known in the art. In some embodiments, the TILs are administered as a single intra-arterial or intravenous infusion, which preferably lasts approximately 30 to 60 minutes. Other suitable routes of administration include intraperitoneal, intrathecal, and intralymphatic administration.

### VIII. Further Gen 2, Gen 3, and Other TIL Manufacturing Process Embodiments

### A. PBMC Feeder Cell Ratios

In some embodiments, the culture media used in expansion methods described herein (see for example, FIG. 8 (in particular, e.g., FIG. 8A and/or FIG. 8B and/or FIG. 8C and/or FIG. 8D)) include an anti-CD3 antibody e.g. OKT-3. An anti-CD3 antibody in combination with IL-2 induces T cell activation and cell division in the TIL population. This effect can be seen with full length antibodies as well as Fab and F(ab′)2 fragments, with the former being generally preferred; see, e.g., Tsoukas et al., J Immunol. 1985, 135, 1719, hereby incorporated by reference in its entirety.

In some embodiments, the number of PBMC feeder layers is calculated as follows:
A. Volume of a T-cell (10 μm diameter): V=(4/3) πr³=523.6 μm³
B. Column of G-REX-100 (M) with a 40 μm (4 cells) height: V=(4/3) πr3=4×10¹² μm³
C. Number of cells required to fill column B: 4×10¹² μm³/523.6 μm³=7.6×10⁸ m³*0.64=4.86×10⁸
D. Number cells that can be optimally activated in 4D space: 4.86×10⁸/24=20.25×10⁶
E. Number of feeders and TIL extrapolated to G-REX-500: TIL: 100×10⁶ and Feeder: 2.5×10⁹

In this calculation, an approximation of the number of mononuclear cells required to provide an icosahedral geometry for activation of TIL in a cylinder with a 100 cm² base is used. The calculation derives the experimental result of ˜5×10⁸ for threshold activation of T-cells which closely mirrors NCI experimental data, as described in Jin, et. al., J. Immunother. 2012, 35, 283-292. In (C), the multiplier (0.64) is the random packing density for equivalent spheres as calculated by Jaeger and Nagel, Science, 1992, 255, 1523-3. In (D), the divisor 24 is the number of equivalent spheres that could contact a similar object in 4-dimensional space or “the Newton number” as described in Musin, Russ. Math. Surv., 2003, 58, 794-795.

In some embodiments, the number of antigen-presenting feeder cells exogenously supplied during the priming first expansion is approximately one-half the number of antigen-presenting feeder cells exogenously supplied during the rapid second expansion. In certain embodiments, the method comprises performing the priming first expansion in a cell culture medium which comprises approximately 50% fewer antigen presenting cells as compared to the cell culture medium of the rapid second expansion.

In other embodiments, the number of antigen-presenting feeder cells (APCs) exogenously supplied during the rapid second expansion is greater than the number of APCs exogenously supplied during the priming first expansion.

In other embodiments, the ratio of the number of APCs exogenously supplied during the rapid second expansion to the number of APCs exogenously supplied during the priming first expansion is selected from a range of from at or about 1.1:1 to at or about 20:1.

In other embodiments, the ratio of the number of APCs exogenously supplied during the rapid second expansion to the number of APCs exogenously supplied during the priming first expansion is selected from a range of from at or about 1.1:1 to at or about 10:1.

In other embodiments, the ratio of the number of APCs exogenously supplied during the rapid second expansion to the number of APCs exogenously supplied during the priming first expansion is selected from a range of from at or about 1.1:1 to at or about 9:1.

In other embodiments, the ratio of the number of APCs exogenously supplied during the rapid second expansion to the number of APCs exogenously supplied during the priming first expansion is selected from a range of from at or about 1.1:1 to at or about 8:1.

In other embodiments, the ratio of the number of APCs exogenously supplied during the rapid second expansion to the number of APCs exogenously supplied during the priming first expansion is selected from a range of from at or about 1.1:1 to at or about 7:1.

In other embodiments, the ratio of the number of APCs exogenously supplied during the rapid second expansion to the number of APCs exogenously supplied during the priming first expansion is selected from a range of from at or about 1.1:1 to at or about 6:1.

In other embodiments, the ratio of the number of APCs exogenously supplied during the rapid second expansion to the number of APCs exogenously supplied during the priming first expansion is selected from a range of from at or about 1.1:1 to at or about 5:1.

In other embodiments, the ratio of the number of APCs exogenously supplied during the rapid second expansion to the number of APCs exogenously supplied during the priming first expansion is selected from a range of from at or about 1.1:1 to at or about 4:1.

In other embodiments, the ratio of the number of APCs exogenously supplied during the rapid second expansion to the number of APCs exogenously supplied during the priming first expansion) is selected from a range of from at or about 1.1:1 to at or about 3:1.

In other embodiments, the ratio of the number of APCs exogenously supplied during the rapid second expansion to the number of APCs exogenously supplied during the priming first expansion is selected from a range of from at or about 1.1:1 to at or about 2.9:1.

In other embodiments, the ratio of the number of APCs exogenously supplied during the rapid second expansion to the number of APCs exogenously supplied during the priming first expansion is selected from a range of from at or about 1.1:1 to at or about 2.8:1.

In other embodiments, the ratio of the number of APCs exogenously supplied during the rapid second expansion to the number of APCs exogenously supplied during the priming first expansion is selected from a range of from at or about 1.1:1 to at or about 2.7:1.

In other embodiments, the ratio of the number of APCs exogenously supplied during the rapid second expansion to the number of APCs exogenously supplied during the priming first expansion is selected from a range of from at or about 1.1:1 to at or about 2.6:1.

In other embodiments, the ratio of the number of APCs exogenously supplied during the rapid second expansion to the number of APCs exogenously supplied during the priming first expansion is selected from a range of from at or about 1.1:1 to at or about 2.5:1.

In other embodiments, the ratio of the number of APCs exogenously supplied during the rapid second expansion to the number of APCs exogenously supplied during the priming first expansion is selected from a range of from at or about 1.1:1 to at or about 2.4:1.

In other embodiments, the ratio of the number of APCs exogenously supplied during the rapid second expansion to the number of APCs exogenously supplied during the priming first expansion is selected from a range of from at or about 1.1:1 to at or about 2.3:1.

In other embodiments, the ratio of the number of APCs exogenously supplied during the rapid second expansion to the number of APCs exogenously supplied during the priming first expansion is selected from a range of from at or about 1.1:1 to at or about 2.2:1.

In other embodiments, the ratio of the number of APCs exogenously supplied during the rapid second expansion to the number of APCs exogenously supplied during the priming first expansion is selected from a range of from at or about 1.1:1 to at or about 2.1:1.

In other embodiments, the ratio of the number of APCs exogenously supplied during the rapid second expansion to the number of APCs exogenously supplied during the priming first expansion is selected from a range of from at or about 1.1:1 to at or about 2:1.

In other embodiments, the ratio of the number of APCs exogenously supplied during the rapid second expansion to the number of APCs exogenously supplied during the priming first expansion is selected from a range of from at or about 2:1 to at or about 10:1.

In other embodiments, the ratio of the number of APCs exogenously supplied during the rapid second expansion to the number of APCs exogenously supplied during the priming first expansion is selected from a range of from at or about 2:1 to at or about 5:1.

In other embodiments, the ratio of the number of APCs exogenously supplied during the rapid second expansion to the number of APCs exogenously supplied during the priming first expansion is selected from a range of from at or about 2:1 to at or about 4:1.

In other embodiments, the ratio of the number of APCs exogenously supplied during the rapid second expansion to the number of APCs exogenously supplied during the priming first expansion is selected from a range of from at or about 2:1 to at or about 3:1.

In other embodiments, the ratio of the number of APCs exogenously supplied during the rapid second expansion to the number of APCs exogenously supplied during the priming first expansion is selected from a range of from at or about 2:1 to at or about 2.9:1.

In other embodiments, the ratio of the number of APCs exogenously supplied during the rapid second expansion to the number of APCs exogenously supplied during the priming first expansion is selected from a range of from at or about 2:1 to at or about 2.8:1.

In other embodiments, the ratio of the number of APCs exogenously supplied during the rapid second expansion to the number of APCs exogenously supplied during the priming first expansion is selected from a range of from at or about 2:1 to at or about 2.7:1.

In other embodiments, the ratio of the number of APCs exogenously supplied during the rapid second expansion to the number of APCs exogenously supplied during the priming first expansion is selected from a range of from at or about 2:1 to at or about 2.6:1.

In other embodiments, the ratio of the number of APCs exogenously supplied during the rapid second expansion to the number of APCs exogenously supplied during the priming first expansion is selected from a range of from at or about 2:1 to at or about 2.5:1.

In other embodiments, the ratio of the number of APCs exogenously supplied during the rapid second expansion to the number of APCs exogenously supplied during the priming first expansion is selected from a range of from at or about 2:1 to at or about 2.4:1.

In other embodiments, the ratio of the number of APCs exogenously supplied during the rapid second expansion to the number of APCs exogenously supplied during the priming first expansion is selected from a range of from at or about 2:1 to at or about 2.3:1.

In other embodiments, the ratio of the number of APCs exogenously supplied during the rapid second expansion to the number of APCs exogenously supplied during the priming first expansion is selected from a range of from at or about about 2:1 to at or about 2.2:1.

In other embodiments, the ratio of the number of APCs exogenously supplied during the rapid second expansion to the number of APCs exogenously supplied during the priming first expansion is selected from a range of from at or about 2:1 to at or about 2.1:1.

In other embodiments, the ratio of the number of APCs exogenously supplied during the rapid second expansion to the number of APCs exogenously supplied during the priming first expansion is at or about 2:1.

In other embodiments, the ratio of the number of APCs exogenously supplied during the rapid second expansion to the number of APCs exogenously supplied during the priming first expansion is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In other embodiments, the number of APCs exogenously supplied during the priming first expansion is at or about 1×10⁸, 1.1×10′, 1.2×10⁸, 1.3×10⁸, 1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸ or 3.5×10⁸ APCs, and the number of APCs exogenously supplied during the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs.

In other embodiments, the number of APCs exogenously supplied during the priming first expansion is selected from the range of at or about 1.5×10⁸ APCs to at or about 3×10⁸ APCs, and the number of APCs exogenously supplied during the rapid second expansion is selected from the range of at or about 4×10⁸ APCs to at or about 7.5×10⁸ APCs.

In other embodiments, the number of APCs exogenously supplied during the priming first expansion is selected from the range of at or about 2×10⁸ APCs to at or about 2.5×10⁸ APCs, and the number of APCs exogenously supplied during the rapid second expansion is selected from the range of at or about 4.5×10⁸ APCs to at or about 5.5×10⁸ APCs.

In other embodiments, the number of APCs exogenously supplied during the priming first expansion is at or about 2.5×10⁸ APCs, and the number of APCs exogenously supplied during the rapid second expansion is at or about 5×10⁸ APCs.

In some embodiments, the number of APCs (including, for example, PBMCs) added at day 0 of the priming first expansion is approximately one-half of the number of PBMCs added at day 7 of the priming first expansion (e.g., day 7 of the method). In certain embodiments, the method comprises adding antigen presenting cells at day 0 of the priming first expansion to the first population of TILs and adding antigen presenting cells at day 7 to the second population of TILs, wherein the number of antigen presenting cells added at day 0 is approximately 50% of the number of antigen presenting cells added at day 7 of the priming first expansion (e.g., day 7 of the method).

In other embodiments, the number of APCs (including, for example, PBMCs) exogenously supplied at day 7 of the rapid second expansion is greater than the number of PBMCs exogenously supplied at day 0 of the priming first expansion.

In other embodiments, the APCs exogenously supplied in the priming first expansion are seeded in the culture flask at a density selected from a range of at or about 1.0×10⁶ APCs/cm² to at or about 4.5×10⁶ APCs/cm².

In other embodiments, the APCs exogenously supplied in the priming first expansion are seeded in the culture flask at a density selected from a range of at or about 1.5×10⁶ APCs/cm² to at or about 3.5×10⁶ APCs/cm².

In other embodiments, the APCs exogenously supplied in the priming first expansion are seeded in the culture flask at a density selected from a range of at or about 2×10⁶ APCs/cm² to at or about 3×10⁶ APCs/cm².

In other embodiments, the APCs exogenously supplied in the priming first expansion are seeded in the culture flask at a density of at or about 2×10⁶ APCs/cm².

In other embodiments, the APCs exogenously supplied in the priming first expansion are seeded in the culture flask at a density of at or about 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶ 3.1×10⁶, 3.2×10⁶, 3.3×10⁶, 3.4×10⁶, 3.5×10⁶, 3.6×10⁶, 3.7×10⁶ 3.8×10⁶, 3.9×10⁶ 4×10⁶ 4.1×10⁶, 4.2×10⁶, 4.3×10⁶, 4.4×10⁶ or 4.5×10⁶ APCs/cm².

In other embodiments, the APCs exogenously supplied in the rapid second expansion are seeded in the culture flask at a density selected from a range of at or about 2.5×10⁶ APCs/cm² to at or about 7.5×10⁶ APCs/cm².

In other embodiments, the APCs exogenously supplied in the rapid second expansion are seeded in the culture flask at a density selected from a range of at or about 3.5×10⁶ APCs/cm² to about 6.0×10⁶ APCs/cm².

In other embodiments, the APCs exogenously supplied in the rapid second expansion are seeded in the culture flask at a density selected from a range of at or about 4.0×10⁶ APCs/cm² to about 5.5×10⁶ APCs/cm².

In other embodiments, the APCs exogenously supplied in the rapid second expansion are seeded in the culture flask at a density selected from a range of at or about 4.0×10⁶ APCs/cm².

In other embodiments, the APCs exogenously supplied in the rapid second expansion are seeded in the culture flask at a density of at or about 2.5×10⁶ APCs/cm², 2.6×10⁶ APCs/cm², 2.7×10⁶ APCs/cm², 2.8×10⁶, 2.9×10⁶, 3×10⁶ 3.1×10⁶, 3.2×10⁶, 3.3×10⁶, 3.4×10⁶, 3.5×10⁶, 3.6×10⁶, 3.7×10⁶ 3.8×10⁶, 3.9×10⁶ 4×10⁶ 4.1×10⁶, 4.2×10⁶ 4.3×10⁶4.4×10⁶, 4.5×10⁶, 4.6×10⁶, 4.7×10⁶, 4.8×10⁶, 4.9×10⁶ 5×10⁶ 5.1×10⁶, 5.2×10⁶ 5.3×10⁶5.4×10⁶, 5.5×10⁶, 5.6×10⁶, 5.7×10⁶ 5.8×10⁶, 5.9×10⁶ 6×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7×10⁶ 7.1×10⁶, 7.2×10⁶ 7.3×10⁶, 7.4×10⁶ or 7.5×10⁶ APCs/cm².

In other embodiments, the APCs exogenously supplied in the priming first expansion are seeded in the culture flask at a density of at or about 1.0×10⁶, 1.1×10⁶, 1.2×10⁶, 1.3×10⁶, 1.4×10⁶, 1.5×10⁶, 1.6×10⁶, 1.7×10⁶, 1.8×10⁶, 1.9×10⁶, 2×10⁶, 2.1×10⁶, 2.2×10⁶, 2.3×10⁶, 2.4×10⁶, 2.5×10⁶, 2.6×10⁶, 2.7×10⁶, 2.8×10⁶, 2.9×10⁶, 3×10⁶ 3.1×10⁶, 3.2×10⁶, 3.3×10⁶, 3.4×10⁶, 3.5×10⁶, 3.6×10⁶, 3.7×10⁶ 3.8×10⁶, 3.9×10⁶ 4×10⁶ 4.1×10⁶, 4.2×10⁶, 4.3×10⁶, 4.4×10⁶ or 4.5×10⁶ APCs/cm² and the APCs exogenously supplied in the rapid second expansion are seeded in the culture flask at a density of at or about 2.5×10⁶ APCs/cm², 2.6×10⁶ APCs/cm², 2.7×10⁶ APCs/cm², 2.8×10⁶, 2.9×10⁶, 3×10⁶ 3.1×10⁶, 3.2×10⁶, 3.3×10⁶ 3.4×10⁶, 3.5×10⁶ 3.6×10⁶ 3.7×10⁶ 3.8×10⁶, 3.9×10⁶ 4×10⁶ 4.1×10⁶, 4.2×10⁶, 4.3×10⁶ 4.4×10⁶ 4.5×10⁶ 4.6×10⁶ 4.7×10⁶, 4.8×10⁶, 4.9×10⁶ 5×10⁶ 5.1×10⁶, 5.2×10⁶, 5.3×10⁶ 5.4×10⁶ 5.5×10⁶ 5.6×10⁶ 5.7×10⁶ 5.8×10⁶, 5.9×10⁶ 6×10⁶, 6.1×10⁶, 6.2×10⁶, 6.3×10⁶, 6.4×10⁶, 6.5×10⁶, 6.6×10⁶, 6.7×10⁶, 6.8×10⁶, 6.9×10⁶, 7×10⁶ 7.1×10⁶, 7.2×106, 7.3×106, 7.4×106 or 7.5×10⁶ APCs/cm².

In other embodiments, the APCs exogenously supplied in the priming first expansion are seeded in the culture flask at a density selected from a range of at or about 1.0×10⁶ APCs/cm² to at or about 4.5×10⁶ APCs/cm², and the APCs exogenously supplied in the rapid second expansion are seeded in the culture flask at a density selected from a range of at or about 2.5×10⁶ APCs/cm² to at or about 7.5×10⁶ APCs/cm².

In other embodiments, the APCs exogenously supplied in the priming first expansion are seeded in the culture flask at a density selected from a range of at or about 1.5×10⁶ APCs/cm² to at or about 3.5×10⁶ APCs/cm², and the APCs exogenously supplied in the rapid second expansion are seeded in the culture flask at a density selected from a range of at or about 3.5×10⁶ APCs/cm² to at or about 6×10⁶ APCs/cm².

In other embodiments, the APCs exogenously supplied in the priming first expansion are seeded in the culture flask at a density selected from a range of at or about 2×10⁶ APCs/cm² to at or about 3×10⁶ APCs/cm², and the APCs exogenously supplied in the rapid second expansion are seeded in the culture flask at a density selected from a range of at or about 4×10⁶ APCs/cm² to at or about 5.5×10⁶ APCs/cm².

In other embodiments, the APCs exogenously supplied in the priming first expansion are seeded in the culture flask at a density at or about 2×10⁶ APCs/cm² and the APCs exogenously supplied in the rapid second expansion are seeded in the culture flask at a density of at or about 4×10⁶ APCs/cm².

In other embodiments, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 20:1.

In other embodiments, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 10:1.

In other embodiments, the ratio of the number of APCs (including, for example, PBMCs) exogenously supplied at day 7 of the rapid second expansion to the number of PBMCs exogenously supplied at day 0 of the priming first expansion is selected from a range of from at or about 1.1:1 to at or about 9:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 8:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 7:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 6:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 5:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 4:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 3:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.9:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.8:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.7:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.6:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.5:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.4:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.3:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.2:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2.1:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 1.1:1 to at or about 2:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 10:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 5:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 4:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 3:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.9:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.8:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.7:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.6:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.5:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.4:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.3:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about about 2:1 to at or about 2.2:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is selected from a range of from at or about 2:1 to at or about 2.1:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is at or about 2:1.

In other embodiments, the ratio of the number of APCs (including, for example, PBMCs) exogenously supplied at day 7 of the rapid second expansion to the number of APCs (including, for example, PBMCs) exogenously supplied at day 0 of the priming first expansion is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In other embodiments, the number of APCs (including, for example, PBMCs) exogenously supplied at day 0 of the priming first expansion is at or about 1×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸ or 3.5×10⁸ APCs (including, for example, PBMCs), and the number of APCs (including, for example, PBMCs) exogenously supplied at day 7 of the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs (including, for example, PBMCs).

In other embodiments, the number of APCs (including, for example, PBMCs) exogenously supplied at day 0 of the priming first expansion is selected from the range of at or about 1×10⁸ APCs (including, for example, PBMCs) to at or about 3.5×10⁸ APCs (including, for example, PBMCs), and the number of APCs (including, for example, PBMCs) exogenously supplied at day 7 of the rapid second expansion is selected from the range of at or about 3.5×10⁸ APCs (including, for example, PBMCs) to at or about 1×10⁹ APCs (including, for example, PBMCs).

In other embodiments, the number of APCs (including, for example, PBMCs) exogenously supplied at day 0 of the priming first expansion is selected from the range of at or about 1.5×10⁸ APCs to at or about 3×10⁸ APCs (including, for example, PBMCs), and the number of APCs (including, for example, PBMCs) exogenously supplied at day 7 of the rapid second expansion is selected from the range of at or about 4×10⁸ APCs (including, for example, PBMCs) to at or about 7.5×10⁸ APCs (including, for example, PBMCs).

In other embodiments, the number of APCs (including, for example, PBMCs) exogenously supplied at day 0 of the priming first expansion is selected from the range of at or about 2×10⁸ APCs (including, for example, PBMCs) to at or about 2.5×10⁸ APCs (including, for example, PBMCs), and the number of APCs (including, for example, PBMCs) exogenously supplied at day 7 of the rapid second expansion is selected from the range of at or about 4.5×10⁸ APCs (including, for example, PBMCs) to at or about 5.5×10⁸ APCs (including, for example, PBMCs).

In other embodiments, the number of APCs (including, for example, PBMCs) exogenously supplied at day 0 of the priming first expansion is at or about 2.5×10⁸ APCs (including, for example, PBMCs) and the number of APCs (including, for example, PBMCs) exogenously supplied at day 7 of the rapid second expansion is at or about 5×10⁸ APCs (including, for example, PBMCs)

In some embodiments, the number of layers of APCs (including, for example, PBMCs) added at day 0 of the priming first expansion is approximately one-half of the number of layers of APCs (including, for example, PBMCs) added at day 7 of the rapid second expansion. In certain embodiments, the method comprises adding antigen presenting cell layers at day 0 of the priming first expansion to the first population of TILs and adding antigen presenting cell layers at day 7 to the second population of TILs, wherein the number of antigen presenting cell layer added at day 0 is approximately 50% of the number of antigen presenting cell layers added at day 7.

In other embodiments, the number of layers of APCs (including, for example, PBMCs) exogenously supplied at day 7 of the rapid second expansion is greater than the number of layers of APCs (including, for example, PBMCs) exogenously supplied at day 0 of the priming first expansion.

In other embodiments, day 0 of the priming first expansion occurs in the presence of layered APCs (including, for example, PBMCs) with an average thickness of at or about 2 cell layers and day 7 of the rapid second expansion occurs in the presence of layered APCs (including, for example, PBMCs) with an average thickness of at or about 4 cell layers.

In other embodiments, day 0 of the priming first expansion occurs in the presence of layered APCs (including, for example, PBMCs) with an average thickness of at or about one cell layer and day 7 of the rapid second expansion occurs in the presence of layered APCs (including, for example, PBMCs) with an average thickness of at or about 3 cell layers.

In other embodiments, day 0 of the priming first expansion occurs in the presence of layered APCs (including, for example, PBMCs) with an average thickness of at or about 1.5 cell layers to at or about 2.5 cell layers and day 7 of the rapid second expansion occurs in the presence of layered APCs (including, for example, PBMCs) with an average thickness of at or about 3 cell layers.

In other embodiments, day 0 of the priming first expansion occurs in the presence of layered APCs (including, for example, PBMCs) with an average thickness of at or about one cell layer and day 7 of the rapid second expansion occurs in the presence of layered APCs (including, for example, PBMCs) with an average thickness of at or about 2 cell layers.

In other embodiments, day 0 of the priming first expansion occurs in the presence of layered APCs (including, for example, PBMCs) with an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers and day 7 of the rapid second expansion occurs in the presence of layered APCs (including, for example, PBMCs) with an average thickness of at or about 3.1, 3.2, 3.3, 3.4, 3.5, 3.6, 3.7, 3.8, 3.9, 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In other embodiments, day 0 of the priming first expansion occurs in the presence of layered APCs (including, for example, PBMCs) with an average thickness of at or about 1 cell layer to at or about 2 cell layers and day 7 of the rapid second expansion occurs in the presence of layered APCs (including, for example, PBMCs) with an average thickness of at or about 3 cell layers to at or about 10 cell layers.

In other embodiments, day 0 of the priming first expansion occurs in the presence of layered APCs (including, for example, PBMCs) with an average thickness of at or about 2 cell layers to at or about 3 cell layers and day 7 of the rapid second expansion occurs in the presence of layered APCs (including, for example, PBMCs) with an average thickness of at or about 4 cell layers to at or about 8 cell layers.

In other embodiments, day 0 of the priming first expansion occurs in the presence of layered APCs (including, for example, PBMCs) with an average thickness of at or about 2 cell layers and day 7 of the rapid second expansion occurs in the presence of layered APCs (including, for example, PBMCs) with an average thickness of at or about 4 cell layers to at or about 8 cell layers.

In other embodiments, day 0 of the priming first expansion occurs in the presence of layered APCs (including, for example, PBMCs) with an average thickness of at or about 1, 2 or 3 cell layers and day 7 of the rapid second expansion occurs in the presence of layered APCs (including, for example, PBMCs) with an average thickness of at or about 3, 4, 5, 6, 7, 8, 9 or 10 cell layers.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:10.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:8.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:7.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:6.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:5.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:4.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:3.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.1 to at or about 1:2.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.2 to at or about 1:8.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.3 to at or about 1:7.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.4 to at or about 1:6.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.5 to at or about 1:5.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.6 to at or about 1:4.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.7 to at or about 1:3.5.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.8 to at or about 1:3.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from the range of at or about 1:1.9 to at or about 1:2.5.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is at or about 1:2.

In other embodiments, day 0 of the priming first expansion occurs in the presence of layered APCs (including, for example, PBMCs) with a first average thickness equal to a first number of layers of APCs (including, for example, PBMCs) and day 7 of the rapid second expansion occurs in the presence of layered APCs (including, for example, PBMCs) with a second average thickness equal to a second number of layers of APCs (including, for example, PBMCs), wherein the ratio of the first number of layers of APCs (including, for example, PBMCs) to the second number of layers of APCs (including, for example, PBMCs) is selected from at or about 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9 or 1:10.

In some embodiments, the number of APCs in the priming first expansion is selected from the range of about 1.0×10⁶ APCs/cm² to about 4.5×10⁶ APCs/cm², and the number of APCs in the rapid second expansion is selected from the range of about 2.5×10⁶ APCs/cm² to about 7.5×10⁶ APCs/cm².

In some embodiments, the number of APCs in the priming first expansion is selected from the range of about 1.5×10⁶ APCs/cm² to about 3.5×10⁶ APCs/cm², and the number of APCs in the rapid second expansion is selected from the range of about 3.5×10⁶ APCs/cm² to about 6.0×10⁶ APCs/cm².

In some embodiments, the number of APCs in the priming first expansion is selected from the range of about 2.0×10⁶ APCs/cm² to about 3.0×10⁶ APCs/cm², and the number of APCs in the rapid second expansion is selected from the range of about 4.0×10⁶ APCs/cm² to about 5.5×10⁶ APCs/cm².

### A. Optional Cell Medium Components

### 1. Anti-CD3 Antibodies

In some embodiments, the culture media used in expansion methods described herein (see for example, FIGS. 1 and 8 (in particular, e.g., FIG. 8B)) include an anti-CD3 antibody. An anti-CD3 antibody in combination with IL-2 induces T cell activation and cell division in the TIL population. This effect can be seen with full length antibodies as well as Fab and F(ab′)2 fragments, with the former being generally preferred; see, e.g., Tsoukas et al., J Immunol. 1985, 135, 1719, hereby incorporated by reference in its entirety.

As will be appreciated by those in the art, there are a number of suitable anti-human CD3 antibodies that find use in the invention, including anti-human CD3 polyclonal and monoclonal antibodies from various mammals, including, but not limited to, murine, human, primate, rat, and canine antibodies. In some embodiments, the OKT3 anti-CD3 antibody muromonab is used (commercially available from Ortho-McNeil, Raritan, NJ or Miltenyi Biotech, Auburn, CA). See, Table 1.

As will be appreciated by those in the art, there are a number of suitable anti-human CD3 antibodies that find use in the invention, including anti-human CD3 polyclonal and monoclonal antibodies from various mammals, including, but not limited to, murine, human, primate, rat, and canine antibodies. In some embodiments, the OKT3 anti-CD3 antibody muromonab is used (commercially available from Ortho-McNeil, Raritan, NJ or Miltenyi Biotech, Auburn, CA).

### 2. 4-1BB (CD137) Agonists

In some embodiments, the cell culture medium of the priming first expansion and/or the rapid second expansion comprises a TNFRSF agonist. In some embodiments, the TNFRSF agonist is a 4-1BB (CD137) agonist. The 4-1BB agonist may be any 4-1BB binding molecule known in the art. The 4-1BB binding molecule may be a monoclonal antibody or fusion protein capable of binding to human or mammalian 4-1BB. The 4-1BB agonists or 4-1BB binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The 4-1BB agonist or 4-1BB binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to 4-1BB. In some embodiments, the 4-1BB agonist is an antigen binding protein that is a fully human antibody. In some embodiments, the 4-1BB agonist is an antigen binding protein that is a humanized antibody. In some embodiments, 4-1BB agonists for use in the presently disclosed methods and compositions include anti-4-1BB antibodies, human anti-4-1BB antibodies, mouse anti-4-1BB antibodies, mammalian anti-4-1BB antibodies, monoclonal anti-4-1BB antibodies, polyclonal anti-4-1BB antibodies, chimeric anti-4-1BB antibodies, anti-4-1BB adnectins, anti-4-1BB domain antibodies, single chain anti-4-1BB fragments, heavy chain anti-4-1BB fragments, light chain anti-4-1BB fragments, anti-4-1BB fusion proteins, and fragments, derivatives, conjugates, variants, or biosimilars thereof. Agonistic anti-4-1BB antibodies are known to induce strong immune responses. Lee, et al., PLOS One 2013, 8, e69677. In some embodiments, the 4-1BB agonist is an agonistic, anti-4-1BB humanized or fully human monoclonal antibody (i.e., an antibody derived from a single cell line). In some embodiments, the 4-1BB agonist is EU-101 (Eutilex Co. Ltd.), utomilumab, or urelumab, or a fragment, derivative, conjugate, variant, or biosimilar thereof. In some embodiments, the 4-1BB agonist is utomilumab or urelumab, or a fragment, derivative, conjugate, variant, or biosimilar thereof.

In some embodiments, the 4-1BB agonist or 4-1BB binding molecule may also be a fusion protein. In some embodiments, a multimeric 4-1BB agonist, such as a trimeric or hexameric 4-1BB agonist (with three or six ligand binding domains), may induce superior receptor (4-1BBL) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47.

Agonistic 4-1BB antibodies and fusion proteins are known to induce strong immune responses. In some embodiments, the 4-1BB agonist is a monoclonal antibody or fusion protein that binds specifically to 4-1BB antigen in a manner sufficient to reduce toxicity. In some embodiments, the 4-1BB agonist is an agonistic 4-1BB monoclonal antibody or fusion protein that abrogates antibody-dependent cellular toxicity (ADCC), for example NK cell cytotoxicity. In some embodiments, the 4-1BB agonist is an agonistic 4-1BB monoclonal antibody or fusion protein that abrogates antibody-dependent cell phagocytosis (ADCP). In some embodiments, the 4-1BB agonist is an agonistic 4-1BB monoclonal antibody or fusion protein that abrogates complement-dependent cytotoxicity (CDC). In some embodiments, the 4-1BB agonist is an agonistic 4-1BB monoclonal antibody or fusion protein which abrogates Fc region functionality.

In some embodiments, the 4-1BB agonists are characterized by binding to human 4-1BB (SEQ ID NO:40) with high affinity and agonistic activity. In some embodiments, the 4-1BB agonist is a binding molecule that binds to human 4-1BB (SEQ ID NO:40). In some embodiments, the 4-1BB agonist is a binding molecule that binds to murine 4-1BB (SEQ ID NO:41). The amino acid sequences of 4-1BB antigen to which a 4-1BB agonist or binding molecule binds are summarized in Table 13.

TABLE 13
Amino acid sequences of 4-1BB antigens.

| Identifier | Sequence (One-Letter Amino Acid Symbols) | |
|---|---|---|
| SEQ ID NO: 40 | MGNSCYNIVA TLLLVLNFER TRSLQDPCSN CPAGTFCDNN RNQICSPCPP NSFSSAGGQR | 60 |
| human 4-1BB, | TCDICRQCKG VFRTRKECSS TSNAECDCTP GFHCLGAGCS MCEQDCKQGQ ELTKKGCKDC | 120 |
| Tumor necrosis | CFGTENDQKR GICRPWTNCS LDGKSVLVNG TKERDVVCGP SPADLSPGAS SVTPPAPARE | 180 |
| factor receptor | PGHSPQIISF FLALTSTALL FLLFFLTLRF SVVKRGRKKL LYIFKQPFMR PVQTTQEEDG | 240 |
| superfamily, | CSCRFPEEEE GGCEL | 255 |
| member 9 (Homo | | |
| sapiens) | | |
| SEQ ID NO: 41 | MGNNCYNVVV IVLLLVGCEK VGAVQNSCDN CQPGTFCRKY NPVCKSCPPS TFSSIGGQPN | 60 |
| murine 4-1BB, | CNICRVCAGY FRFKKFCSST HNAECECIEG FHCLGPQCTR CEKDCRPGQE LTKQGCKTCS | 120 |
| Tumor necrosis | LGTFNDQNGT GVCRPWTNCS LDGRSVLKTG TTEKDVVCGP PVVSFSPSTT ISVTPEGGPG | 180 |
| factor receptor | GHSLQVLTLF LALTSALLLA LIFITLLFSV LKWIRKKFPH IFKQPFKKTT GAAQEEDACS | 240 |
| superfamily, | CRCPQEEEGG GGGYEL | 256 |
| member 9 (Mus | | |
| musculus) | | |

In some embodiments, the compositions, processes and methods described include a 4-1BB agonist that binds human or murine 4-1BB with a K_{D} of about 100 pM or lower, binds human or murine 4-1BB with a K_{D} of about 90 pM or lower, binds human or murine 4-1BB with a K_{D} of about 80 pM or lower, binds human or murine 4-1BB with a K_{D} of about 70 pM or lower, binds human or murine 4-1BB with a K_{D} of about 60 pM or lower, binds human or murine 4-1BB with a K_{D} of about 50 pM or lower, binds human or murine 4-1BB with a K_{D} of about 40 pM or lower, or binds human or murine 4-1BB with a K_{D} of about 30 pM or lower.

In some embodiments, the compositions, processes and methods described include a 4-1BB agonist that binds to human or murine 4-1BB with a k_{assoc} of about 7.5×10⁵ l/M·s or faster, binds to human or murine 4-1BB with a k_{assoc} of about 7.5×10⁵ l/M·s or faster, binds to human or murine 4-1BB with a k_{assoc} of about 8×10⁵ l/M·s or faster, binds to human or murine 4-1BB with a k_{assoc} of about 8.5×10⁵ l/M·s or faster, binds to human or murine 4-1BB with a k_{assoc} of about 9×10⁵ l/M·s or faster, binds to human or murine 4-1BB with a k_{assoc} of about 9.5×10⁵ l/M·s or faster, or binds to human or murine 4-1BB with a k_{assoc} of about 1×10⁶ l/M·s or faster.

In some embodiments, the compositions, processes and methods described include a 4-1BB agonist that binds to human or murine 4-1BB with a k_{dissoc} of about 2×10⁻⁵ l/s or slower, binds to human or murine 4-1BB with a k_{dissoc} of about 2.1×10⁻⁵ l/s or slower, binds to human or murine 4-1BB with a k_{dissoc} of about 2.2×10⁻⁵ l/s or slower, binds to human or murine 4-1BB with a k_{dissoc} of about 2.3×10⁻⁵ l/s or slower, binds to human or murine 4-1BB with a k_{dissoc} of about 2.4×10⁻⁵ l/s or slower, binds to human or murine 4-1BB with a k_{dissoc} of about 2.5×10⁻⁵ l/s or slower, binds to human or murine 4-1BB with a k_{dissoc} of about 2.6×10⁻⁵ l/s or slower or binds to human or murine 4-1BB with a k_{dissoc} of about 2.7×10⁻⁵ l/s or slower, binds to human or murine 4-1BB with a k_{dissoc} of about 2.8×10⁻⁵ l/s or slower, binds to human or murine 4-1BB with a k_{dissoc} of about 2.9×10⁻⁵ l/s or slower, or binds to human or murine 4-1BB with a k_{dissoc} of about 3×10⁻⁵ l/s or slower.

In some embodiments, the compositions, processes and methods described include a 4-1BB agonist that binds to human or murine 4-1BB with an IC₅₀ of about 10 nM or lower, binds to human or murine 4-1BB with an IC₅₀ of about 9 nM or lower, binds to human or murine 4-1BB with an IC₅₀ of about 8 nM or lower, binds to human or murine 4-1BB with an IC₅₀ of about 7 nM or lower, binds to human or murine 4-1BB with an IC₅₀ of about 6 nM or lower, binds to human or murine 4-1BB with an IC₅₀ of about 5 nM or lower, binds to human or murine 4-1BB with an IC₅₀ of about 4 nM or lower, binds to human or murine 4-1BB with an IC₅₀ of about 3 nM or lower, binds to human or murine 4-1BB with an IC₅₀ of about 2 nM or lower, or binds to human or murine 4-1BB with an IC₅₀ of about 1 nM or lower.

In some embodiments, the 4-1BB agonist is utomilumab, also known as PF-05082566 or MOR-7480, or a fragment, derivative, variant, or biosimilar thereof. Utomilumab is available from Pfizer, Inc. Utomilumab is an immunoglobulin G2-lambda, anti-[Homo sapiens TNFRSF9 (tumor necrosis factor receptor (TNFR) superfamily member 9, 4-1BB, T cell antigen ILA, CD137)], Homo sapiens (fully human) monoclonal antibody. The amino acid sequences of utomilumab are set forth in Table 14. Utomilumab comprises glycosylation sites at Asn59 and Asn292; heavy chain intrachain disulfide bridges at positions 22-96 (V_{H}-V_{L}), 143-199 (C_{H}1-C_{L}), 256-316 (C_{H}2) and 362-420 (C_{H}3); light chain intrachain disulfide bridges at positions 22′-87′ (V_{H}-V_{L}) and 136′-195′ (C_{H}1-C_{L}); interchain heavy chain-heavy chain disulfide bridges at IgG2A isoform positions 218-218, 219-219, 222-222, and 225-225, at IgG2A/B isoform positions 218-130, 219-219, 222-222, and 225-225, and at IgG2B isoform positions 219-130 (2), 222-222, and 225-225; and interchain heavy chain-light chain disulfide bridges at IgG2A isoform positions 130-213′ (2), IgG2A/B isoform positions 218-213′ and 130-213′, and at IgG2B isoform positions 218-213′ (2). The preparation and properties of utomilumab and its variants and fragments are described in U.S. Pat. Nos. 8,821,867; 8,337,850; and 9,468,678, and International Patent Application Publication No. WO 2012/032433 A1, the disclosures of each of which are incorporated by reference herein. Preclinical characteristics of utomilumab are described in Fisher, et al., Cancer Immunolog. & Immunother. 2012, 61, 1721-33. Current clinical trials of utomilumab in a variety of hematological and solid tumor indications include U.S. National Institutes of Health clinicaltrials.gov identifiers NCT02444793, NCT01307267, NCT02315066, and NCT02554812.

In some embodiments, a 4-1BB agonist comprises a heavy chain given by SEQ ID NO:42 and a light chain given by SEQ ID NO:43. In some embodiments, a 4-1BB agonist comprises heavy and light chains having the sequences shown in SEQ ID NO:42 and SEQ ID NO:43, respectively, or antigen binding fragments, Fab fragments, single-chain variable fragments (scFv), variants, or conjugates thereof. In some embodiments, a 4-1BB agonist comprises heavy and light chains that are each at least 99% identical to the sequences shown in SEQ ID NO:42 and SEQ ID NO:43, respectively. In some embodiments, a 4-1BB agonist comprises heavy and light chains that are each at least 98% identical to the sequences shown in SEQ ID NO:42 and SEQ ID NO:43, respectively. In some embodiments, a 4-1BB agonist comprises heavy and light chains that are each at least 97% identical to the sequences shown in SEQ ID NO:42 and SEQ ID NO:43, respectively. In some embodiments, a 4-1BB agonist comprises heavy and light chains that are each at least 96% identical to the sequences shown in SEQ ID NO:42 and SEQ ID NO:43, respectively. In some embodiments, a 4-1BB agonist comprises heavy and light chains that are each at least 95% identical to the sequences shown in SEQ ID NO:42 and SEQ ID NO:43, respectively.

In some embodiments, the 4-1BB agonist comprises the heavy and light chain CDRs or variable regions (VRs) of utomilumab. In some embodiments, the 4-1BB agonist heavy chain variable region (V_{H}) comprises the sequence shown in SEQ ID NO:44, and the 4-1BB agonist light chain variable region (V_{L}) comprises the sequence shown in SEQ ID NO:45, and conservative amino acid substitutions thereof. In some embodiments, a 4-1BB agonist comprises V_{H} and V_{L} regions that are each at least 99% identical to the sequences shown in SEQ ID NO:44 and SEQ ID NO:45, respectively. In some embodiments, a 4-1BB agonist comprises V_{H} and V_{L} regions that are each at least 98% identical to the sequences shown in SEQ ID NO:44 and SEQ ID NO:45, respectively. In some embodiments, a 4-1BB agonist comprises V_{H} and V_{L} regions that are each at least 97% identical to the sequences shown in SEQ ID NO:44 and SEQ ID NO:45, respectively. In some embodiments, a 4-1BB agonist comprises V_{H} and V_{L} regions that are each at least 96% identical to the sequences shown in SEQ ID NO:44 and SEQ ID NO:45, respectively. In some embodiments, a 4-1BB agonist comprises V_{H} and V_{L} regions that are each at least 95% identical to the sequences shown in SEQ ID NO:44 and SEQ ID NO:45, respectively. In some embodiments, a 4-1BB agonist comprises an scFv antibody comprising V_{H} and V_{L} regions that are each at least 99% identical to the sequences shown in SEQ ID NO:44 and SEQ ID NO:45.

In some embodiments, a 4-1BB agonist comprises heavy chain CDR1, CDR2 and CDR3 domains having the sequences set forth in SEQ ID NO:46, SEQ ID NO:47, and SEQ ID NO:48, respectively, and conservative amino acid substitutions thereof, and light chain CDR1, CDR2 and CDR3 domains having the sequences set forth in SEQ ID NO:49, SEQ ID NO:50, and SEQ ID NO:51, respectively, and conservative amino acid substitutions thereof.

In some embodiments, the 4-1BB agonist is a 4-1BB agonist biosimilar monoclonal antibody approved by drug regulatory authorities with reference to utomilumab. In some embodiments, the biosimilar monoclonal antibody comprises an 4-1BB antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is utomilumab. In some embodiments, the one or more post-translational modifications are selected from one or more of: glycosylation, oxidation, deamidation, and truncation. In some embodiments, the biosimilar is a 4-1BB agonist antibody authorized or submitted for authorization, wherein the 4-1BB agonist antibody is provided in a formulation which differs from the formulations of a reference medicinal product or reference biological product, wherein the reference medicinal product or reference biological product is utomilumab. The 4-1BB agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is utomilumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is utomilumab.

TABLE 14
Amino acid sequences for 4-1BB agonist antibodies related to utomilumab.

| Identifier | Sequence (One-Letter Amino Acid Symbols) | |
|---|---|---|
| SEQ ID NO: 42 | EVQLVQSGAE VKKPGESLRI SCKGSGYSFS TYWISWVRQM PGKGLEWMGK IYPGDSYTNY | 60 |
| heavy chain for | SPSFQGQVTI SADKSISTAY LQWSSLKASD TAMYYCARGY GIFDYWGQGT LVTVSSASTK | 120 |
| utomilumab | GPSVFPLAPC SRSTSESTAA LGCLVKDYFP EPVTVSWNSG ALTSGVHTFP AVLQSSGLYS | 180 |
| | LSSVVTVPSS NFGTQTYTCN VDHKPSNTKV DKTVERKCCV ECPPCPAPPV AGPSVFLFPP | 240 |
| | KPKDTLMISR TPEVTCVVVD VSHEDPEVQF NWYVDGVEVH NAKTKPREEQ FNSTFRVVSV | 300 |
| | LTVVHQDWLN GKEYKCKVSN KGLPAPIEKT ISKTKGQPRE PQVYTLPPSR EEMTKNQVSL | 360 |
| | TCLVKGFYPS DIAVEWESNG QPENNYKTTP PMLDSDGSFF LYSKLTVDKS RWQQGNVFSC | 420 |
| | SVMHEALHNH YTQKSLSLSP G | 441 |
| SEQ ID NO: 43 | SYELTQPPSV SVSPGQTASI TCSGDNIGDQ YAHWYQQKPG QSPVLVIYQD KNRPSGIPER | 60 |
| light chain for | FSGSNSGNTA TLTISGTQAM DEADYYCATY TGFGSLAVFG GGTKLTVLGQ PKAAPSVTLF | 120 |
| utomilumab | PPSSEELQAN KATLVCLISD FYPGAVTVAW KADSSPVKAG VETTTPSKQS NNKYAASSYL | 180 |
| | SLTPEQWKSH RSYSCQVTHE GSTVEKTVAP TECS | 214 |
| SEQ ID NO: 44 | EVQLVQSGAE VKKPGESLRI SCKGSGYSFS TYWISWVRQM PGKGLEWMG KIYPGDSYTN | 60 |
| heavy chain | YSPSFQGQVT ISADKSISTA YLQWSSLKAS DTAMYYCARG YGIFDYWGQ GTLVTVSS | 118 |
| variable region | | |
| for utomilumab | | |
| SEQ ID NO: 45 | SYELTQPPSV SVSPGQTASI TCSGDNIGDQ YAHWYQQKPG QSPVLVIYQD KNRPSGIPER | 60 |
| light chain | FSGSNSGNTA TLTISGTQAM DEADYYCATY TGFGSLAVFG GGTKLTVL | 108 |
| for utomilumab | | |
| variable region | | |
| for utomilumab | | |
| SEQ ID NO: 46 | STYWIS | 6 |
| heavy chain CDR1 | | |
| for utomilumab | | |
| SEQ ID NO: 47 | KIYPGDSYTN YSPSFQG | 17 |
| heavy chain CDR2 | | |
| for utomilumab | | |
| SEQ ID NO: 48 | RGYGIFDY | 8 |
| heavy chain CDR3 | | |
| for utomilumab | | |
| SEQ ID NO: 49 | SGDNIGDQYA H | 11 |
| light chain CDR1 | | |
| for utomilumab | | |
| SEQ ID NO: 50 | QDKNRPS | 7 |
| light chain CDR2 | | |
| for utomilumab | | |
| SEQ ID NO: 51 | ATYTGFGSLA V | 11 |
| light chain CDR3 | | |
| for utomilumab | | |

In some embodiments, the 4-1BB agonist is the monoclonal antibody urelumab, also known as BMS-663513 and 20H4.9.h4a, or a fragment, derivative, variant, or biosimilar thereof. Urelumab is available from Bristol-Myers Squibb, Inc., and Creative Biolabs, Inc. Urelumab is an immunoglobulin G4-kappa, anti-[Homo sapiens TNFRSF9 (tumor necrosis factor receptor superfamily member 9, 4-1BB, T cell antigen ILA, CD137)], Homo sapiens (fully human) monoclonal antibody. The amino acid sequences of urelumab are set forth in Table 15. Urelumab comprises N-glycosylation sites at positions 298 (and 298″); heavy chain intrachain disulfide bridges at positions 22-95 (V_{H}-V_{L}), 148-204 (C_{H}1-C_{L}), 262-322 (C_{H}2) and 368-426 (C_{H}3) (and at positions 22″-95″, 148″-204″, 262″-322″, and 368″-426″); light chain intrachain disulfide bridges at positions 23′-88′ (V_{H}-V_{L}) and 136′-196′ (C_{H}1-C_{L}) (and at positions 23′″-88′″ and 136′″-196′″); interchain heavy chain-heavy chain disulfide bridges at positions 227-227″ and 230-230″; and interchain heavy chain-light chain disulfide bridges at 135-216′ and 135″-216′″. The preparation and properties of urelumab and its variants and fragments are described in U.S. Pat. Nos. 7,288,638 and 8,962,804, the disclosures of which are incorporated by reference herein. The preclinical and clinical characteristics of urelumab are described in Segal, et al., Clin. Cancer Res. 2016, available at http:/dx.doi.org/10.1158/1078-0432.CCR-16-1272. Current clinical trials of urelumab in a variety of hematological and solid tumor indications include U.S. National Institutes of Health clinicaltrials.gov identifiers NCT01775631, NCT02110082, NCT02253992, and NCT01471210.

In some embodiments, a 4-1BB agonist comprises a heavy chain given by SEQ ID NO:52 and a light chain given by SEQ ID NO:53. In some embodiments, a 4-1BB agonist comprises heavy and light chains having the sequences shown in SEQ ID NO:52 and SEQ ID NO:53, respectively, or antigen binding fragments, Fab fragments, single-chain variable fragments (scFv), variants, or conjugates thereof. In some embodiments, a 4-1BB agonist comprises heavy and light chains that are each at least 99% identical to the sequences shown in SEQ ID NO:52 and SEQ ID NO:53, respectively. In some embodiments, a 4-1BB agonist comprises heavy and light chains that are each at least 98% identical to the sequences shown in SEQ ID NO:52 and SEQ ID NO:53, respectively. In some embodiments, a 4-1BB agonist comprises heavy and light chains that are each at least 97% identical to the sequences shown in SEQ ID NO:52 and SEQ ID NO:53, respectively. In some embodiments, a 4-1BB agonist comprises heavy and light chains that are each at least 96% identical to the sequences shown in SEQ ID NO:52 and SEQ ID NO:53, respectively. In some embodiments, a 4-1BB agonist comprises heavy and light chains that are each at least 95% identical to the sequences shown in SEQ ID NO:52 and SEQ ID NO:53, respectively.

In some embodiments, the 4-1BB agonist comprises the heavy and light chain CDRs or variable regions (VRs) of urelumab. In some embodiments, the 4-1BB agonist heavy chain variable region (V_{H}) comprises the sequence shown in SEQ ID NO:54, and the 4-1BB agonist light chain variable region (V_{L}) comprises the sequence shown in SEQ ID NO:55, and conservative amino acid substitutions thereof. In some embodiments, a 4-1BB agonist comprises V_{H} and V_{L} regions that are each at least 99% identical to the sequences shown in SEQ ID NO:54 and SEQ ID NO:55, respectively. In some embodiments, a 4-1BB agonist comprises V_{H} and V_{L} regions that are each at least 98% identical to the sequences shown in SEQ ID NO:54 and SEQ ID NO:55, respectively. In some embodiments, a 4-1BB agonist comprises V_{H} and V_{L} regions that are each at least 97% identical to the sequences shown in SEQ ID NO:54 and SEQ ID NO:55, respectively. In some embodiments, a 4-1BB agonist comprises V_{H} and V_{L} regions that are each at least 96% identical to the sequences shown in SEQ ID NO:54 and SEQ ID NO:55, respectively. In some embodiments, a 4-1BB agonist comprises V_{H} and V_{L} regions that are each at least 95% identical to the sequences shown in SEQ ID NO:54 and SEQ ID NO:55, respectively. In some embodiments, a 4-1BB agonist comprises an scFv antibody comprising V_{H} and V_{L} regions that are each at least 99% identical to the sequences shown in SEQ ID NO:54 and SEQ ID NO:55.

In some embodiments, a 4-1BB agonist comprises heavy chain CDR1, CDR2 and CDR3 domains having the sequences set forth in SEQ ID NO:56, SEQ ID NO:57, and SEQ ID NO:58, respectively, and conservative amino acid substitutions thereof, and light chain CDR1, CDR2 and CDR3 domains having the sequences set forth in SEQ ID NO:59, SEQ ID NO:60, and SEQ ID NO:61, respectively, and conservative amino acid substitutions thereof.

In some embodiments, the 4-1BB agonist is a 4-1BB agonist biosimilar monoclonal antibody approved by drug regulatory authorities with reference to urelumab. In some embodiments, the biosimilar monoclonal antibody comprises an 4-1BB antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is urelumab. In some embodiments, the one or more post-translational modifications are selected from one or more of glycosylation, oxidation, deamidation, and truncation. In some embodiments, the biosimilar is a 4-1BB agonist antibody authorized or submitted for authorization, wherein the 4-1BB agonist antibody is provided in a formulation which differs from the formulations of a reference medicinal product or reference biological product, wherein the reference medicinal product or reference biological product is urelumab. The 4-1BB agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is urelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is urelumab.

TABLE 15
Amino acid sequences for 4-1BB agonist antibodies related to urelumab.

| Identifier | Sequence (One-Letter Amino Acid Symbols) | |
|---|---|---|
| SEQ ID NO: 52 | QVQLQQWGAG LLKPSETLSL TCAVYGGSFS GYYWSWIRQS PEKGLEWIGE INHGGYVTYN | 60 |
| heavy chain for | PSLESRVTIS VDTSKNQFSL KLSSVTAADT AVYYCARDYG PGNYDWYFDL WGRGTLVTVS | 120 |
| urelumab | SASTKGPSVF PLAPCSRSTS ESTAALGCLV KDYFPEPVTV SWNSGALTSG VHTFPAVLQS | 180 |
| | SGLYSLSSVV TVPSSSLGTK TYTCNVDHKP SNTKVDKRVE SKYGPPCPPC PAPEFLGGPS | 240 |
| | VFLFPPKPKD TLMISRTPEV TCVVVDVSQE DPEVQFNWYV DGVEVHNAKT KPREEQFNST | 300 |
| | YRVVSVLTVL HQDWINGKEY KCKVSNKGLP SSIEKTISKA KGQPREPQVY TLPPSQEEMT | 360 |
| | KNQVSLTCLV KGFYPSDIAV EWESNGQPEN NYKTTPPVLD SDGSFFLYSR LTVDKSRWQE | 420 |
| | GNVFSCSVMH EALHNHYTQK SLSLSLGK | 448 |
| SEQ ID NO: 53 | EIVLTQSPAT LSLSPGERAT LSCRASQSVS SYLAWYQQKP GQAPRLLIYD ASNRATGIPA | 60 |
| light chain for | RFSGSGSGTD FTLTISSLEP EDFAVYYCQQ RSNWPPALTF CGGTKVEIKR TVAAPSVFIF | 120 |
| urelumab | PPSDEQLKSG TASVVCLLNN FYPREAKVQW KVDNALQSGN SQESVTEQDS KDSTYSLSST | 180 |
| | LTLSKADYEK HKVYACEVTH QGLSSPVTKS FNRGEC | 216 |
| SEQ ID NO: 54 | MKHLWFFLLL VAAPRWVLSQ VQLQQWGAGL LKPSETLSLT CAVYGGSFSG YYWSWIRQSP | 60 |
| variable heavy | EKGLEWIGEI NHGGYVTYNP SLESRVTISV DTSKNQFSLK LSSVTAADTA VYYCARDYGP | 120 |
| chain for | | |
| urelumab | | |
| SEQ ID NO: 55 | MEAPAQLLFL LLLWLPDTTG EIVLTQSPAT LSLSPGERAT LSCRASQSVS SYLAWYQQKP | 60 |
| variable light | GQAPRLLIYD ASNRATGIPA RFSGSGSGTD FTLTISSLEP EDFAVYYCQQ | 110 |
| chain for | | |
| urelumab | | |
| SEQ ID NO: 56 | GYYWS | 5 |
| heavy chain CDR1 | | |
| for urelumab | | |
| SEQ ID NO: 57 | EINHGGYVTY NPSLES | 16 |
| heavy chain CDR2 | | |
| for urelumab | | |
| SEQ ID NO: 58 | DYGPGNYDWY FDL | 13 |
| heavy chain CDR3 | | |
| for urelumab | | |
| SEQ ID NO: 59 | RASQSVSSYL A | 11 |
| light chain CDR1 | | |
| for urelumab | | |
| SEQ ID NO: 60 | DASNRAT | 7 |
| light chain CDR2 | | |
| for urelumab | | |
| SEQ ID NO: 61 | QQRSDWPPAL T | 11 |
| light chain CDR3 | | |
| for urelumab | | |

In some embodiments, the 4-1BB agonist is selected from the group consisting of 1D8, 3Elor, 4B4 (BioLegend 309809), H4-1BB-M127 (BD Pharmingen 552532), BBK2 (Thermo Fisher MS621PABX), 145501 (Leinco Technologies B591), the antibody produced by cell line deposited as ATCC No. HB-11248 and disclosed in U.S. Pat. No. 6,974,863, 5F4 (BioLegend 31 1503), C65-485 (BD Pharmingen 559446), antibodies disclosed in U.S. Patent Application Publication No. US 2005/0095244, antibodies disclosed in U.S. Pat. No. 7,288,638 (such as 20H4.9-IgG1 (BMS-663031)), antibodies disclosed in U.S. Pat. No. 6,887,673 (such as 4E9 or BMS-554271), antibodies disclosed in U.S. Pat. No. 7,214,493, antibodies disclosed in U.S. Pat. No. 6,303,121, antibodies disclosed in U.S. Pat. No. 6,569,997, antibodies disclosed in U.S. Pat. No. 6,905,685 (such as 4E9 or BMS-554271), antibodies disclosed in U.S. Pat. No. 6,362,325 (such as 1D8 or BMS-469492; 3H3 or BMS-469497; or 3E1), antibodies disclosed in U.S. Pat. No. 6,974,863 (such as 53A2); antibodies disclosed in U.S. Pat. No. 6,210,669 (such as 1D8, 3B8, or 3E1), antibodies described in U.S. Pat. No. 5,928,893, antibodies disclosed in U.S. Pat. No. 6,303,121, antibodies disclosed in U.S. Pat. No. 6,569,997, antibodies disclosed in International Patent Application Publication Nos. WO 2012/177788, WO 2015/119923, and WO 2010/042433, and fragments, derivatives, conjugates, variants, or biosimilars thereof, wherein the disclosure of each of the foregoing patents or patent application publications is incorporated by reference here.

In some embodiments, the 4-1BB agonist is a 4-1BB agonistic fusion protein described in International Patent Application Publication Nos. WO 2008/025516 A1, WO 2009/007120 A1, WO 2010/003766 A1, WO 2010/010051 A1, and WO 2010/078966 A1; U.S. Patent Application Publication Nos. US 2011/0027218 A1, US 2015/0126709 A1, US 2011/0111494 A1, US 2015/0110734 A1, and US 2015/0126710 A1; and U.S. Pat. Nos. 9,359,420, 9,340,599, 8,921,519, and 8,450,460, the disclosures of which are incorporated by reference herein.

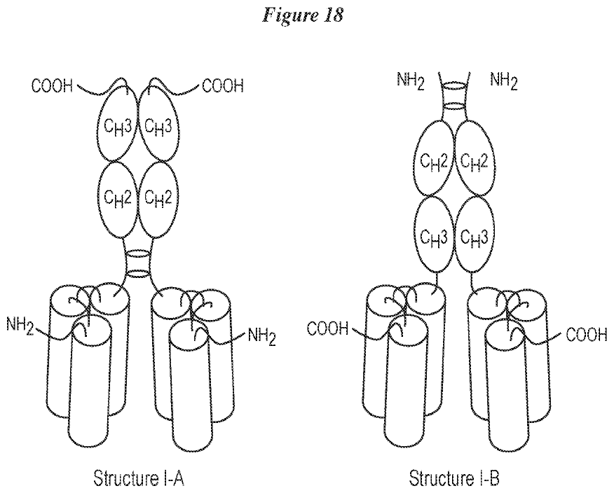

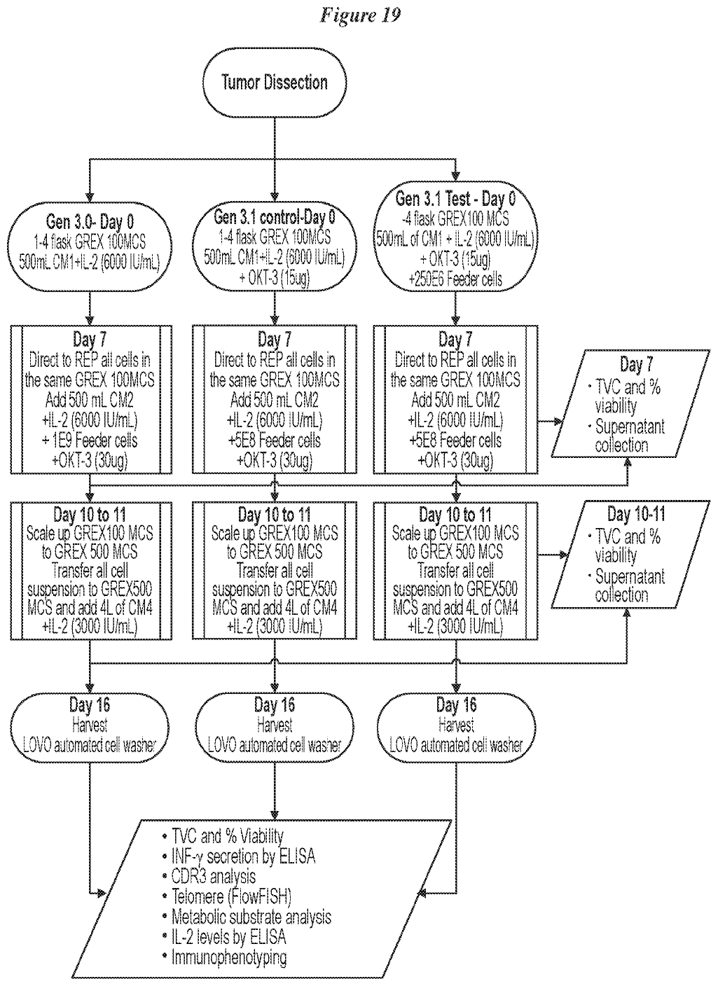

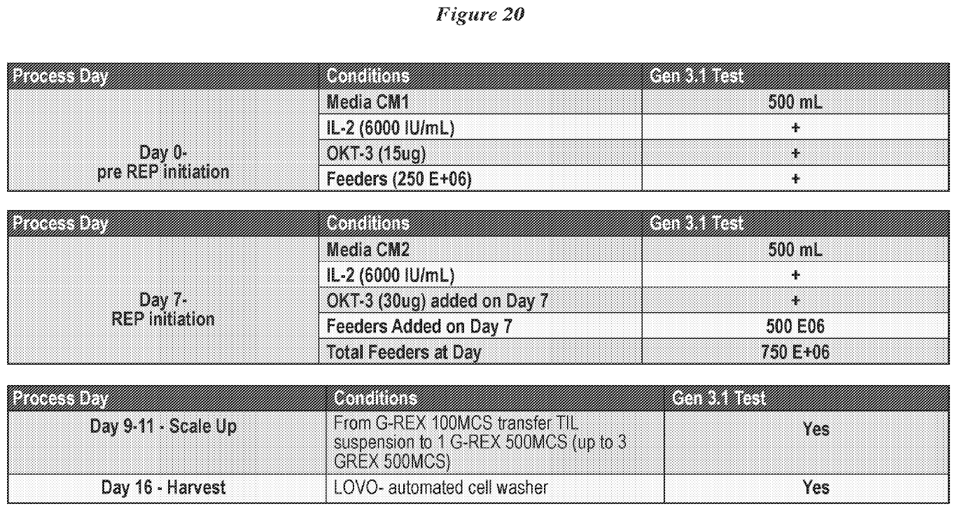

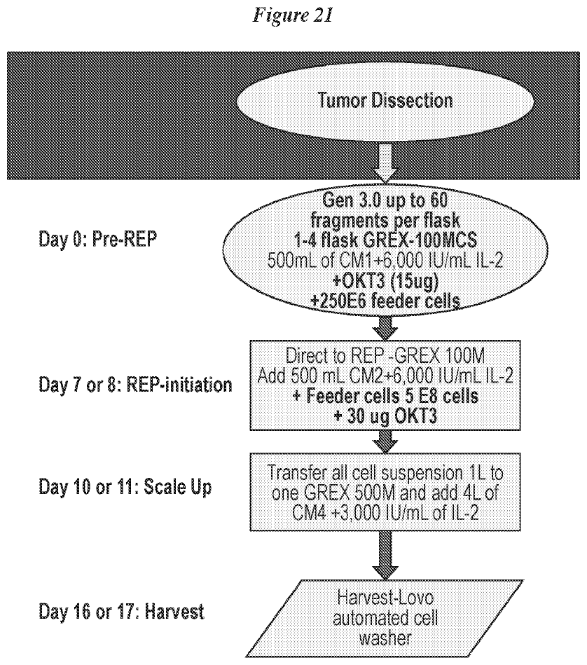

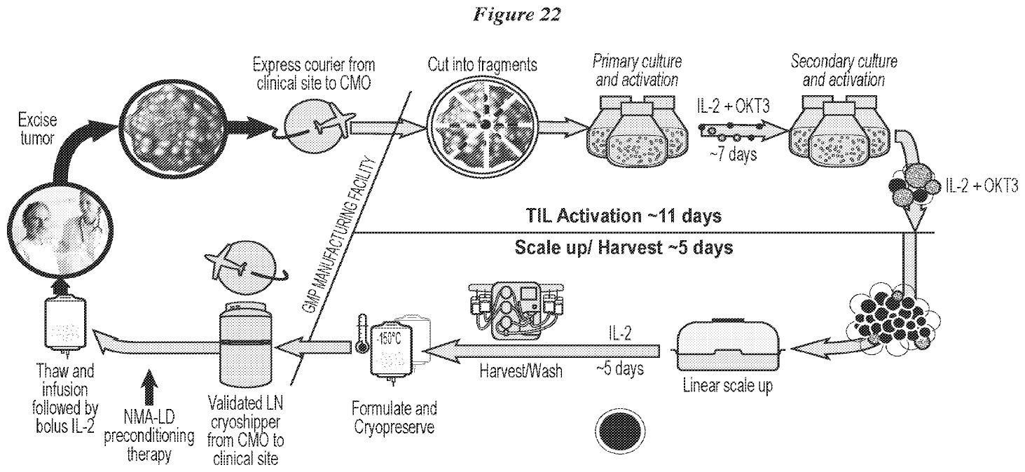

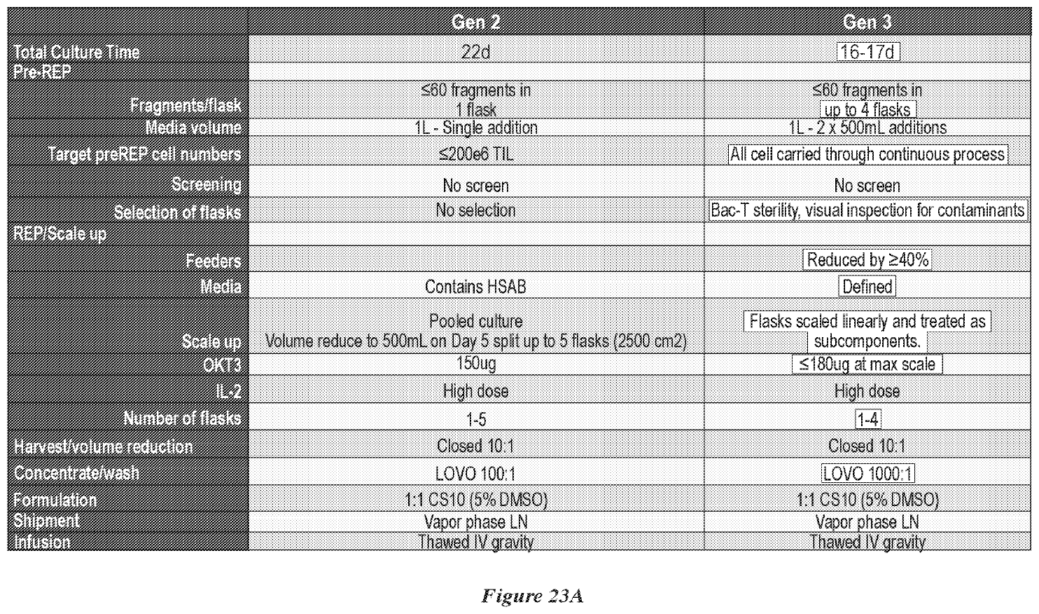

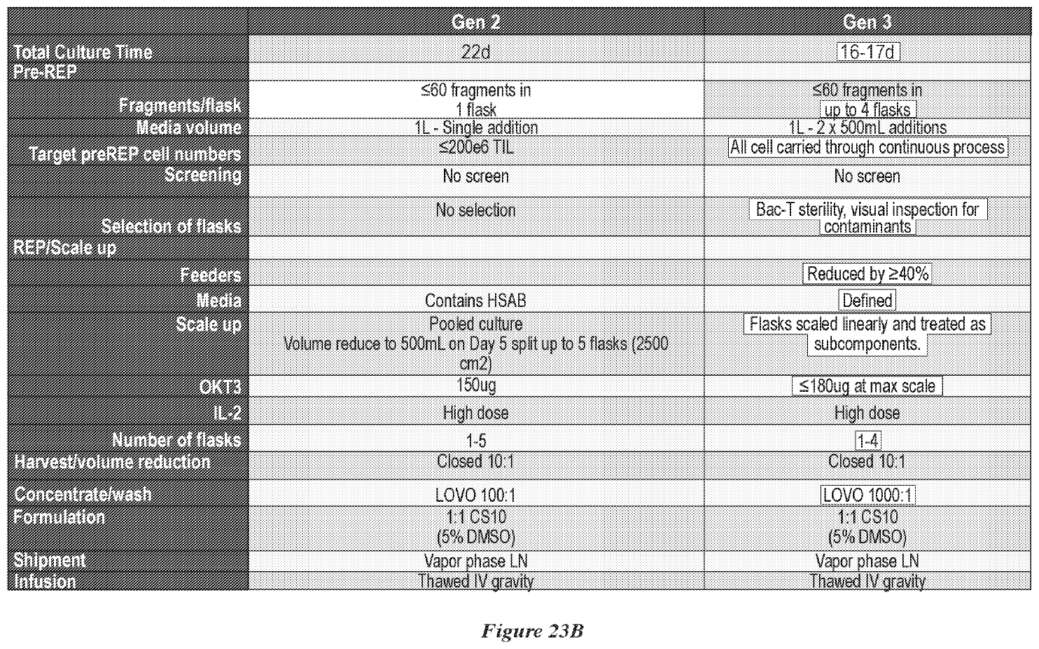

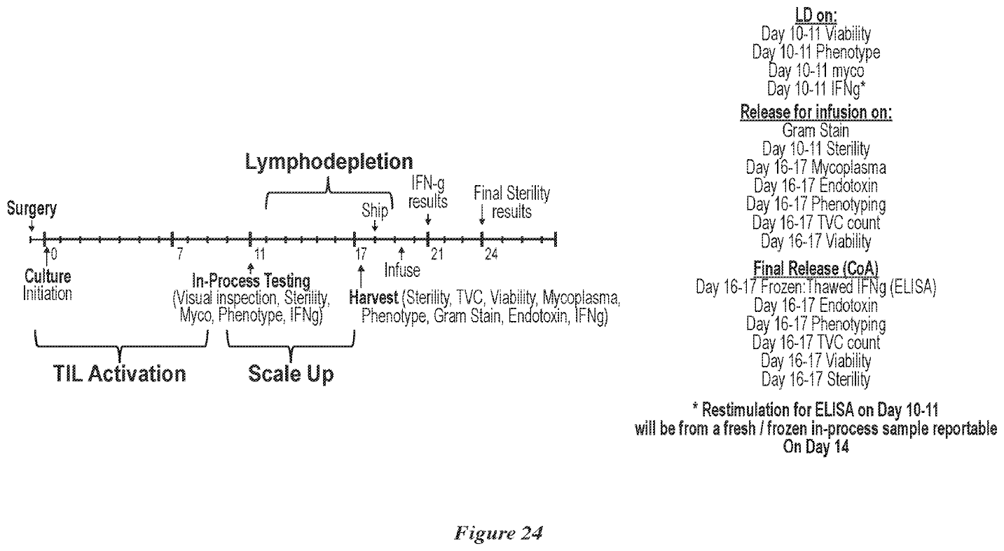

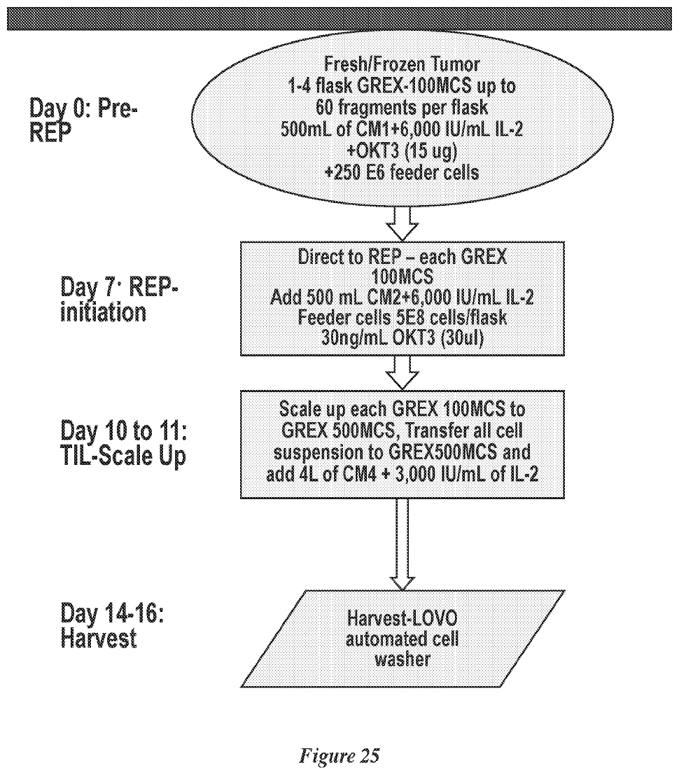

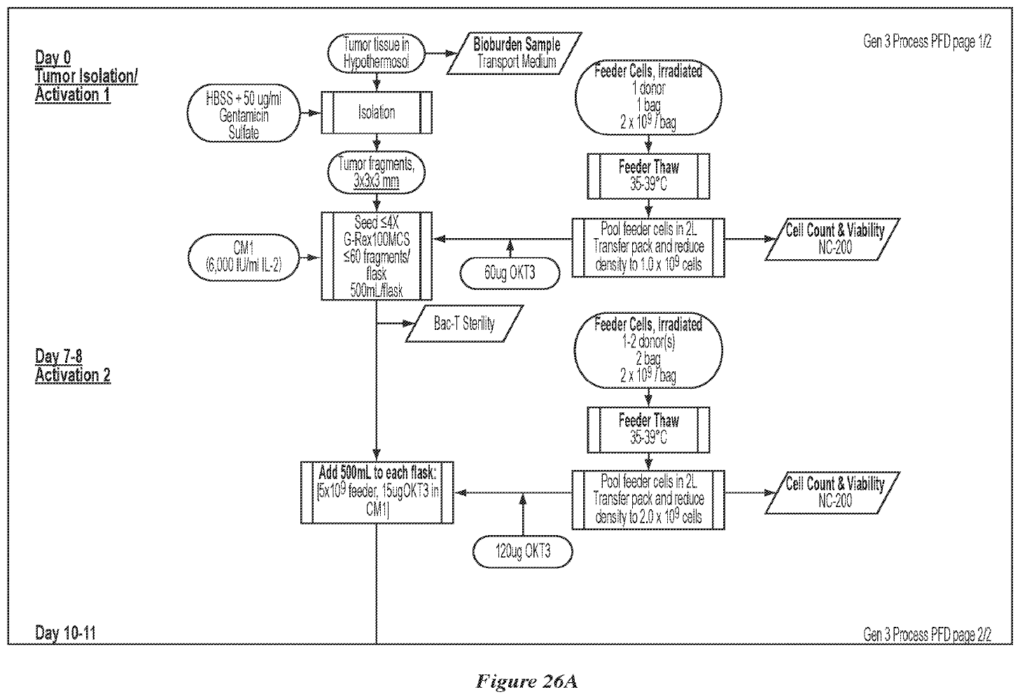

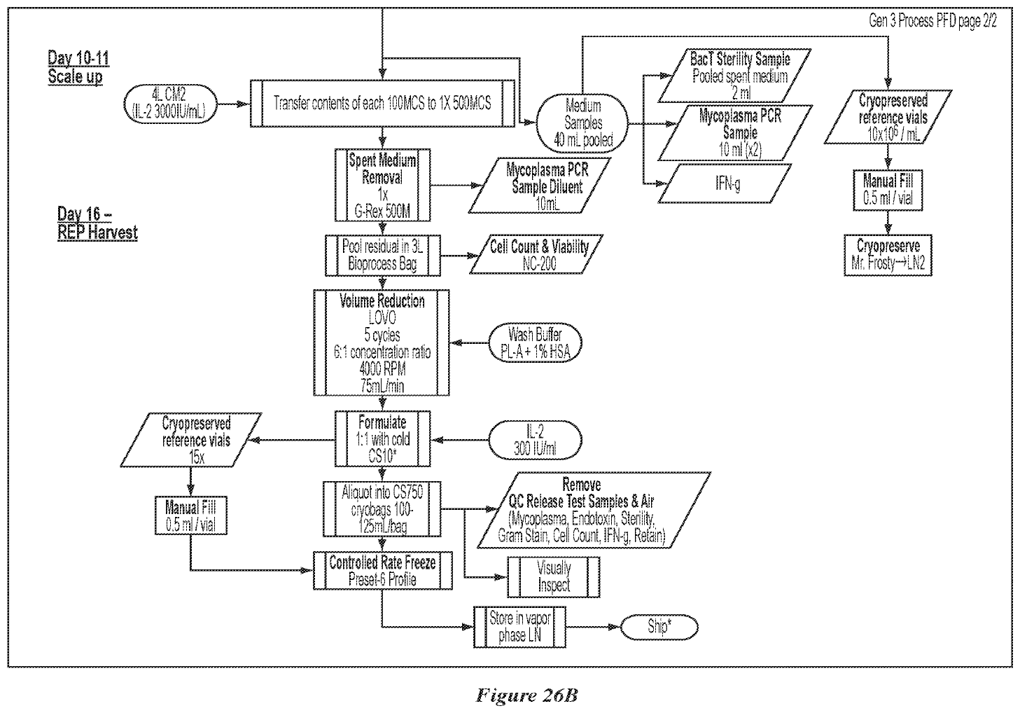

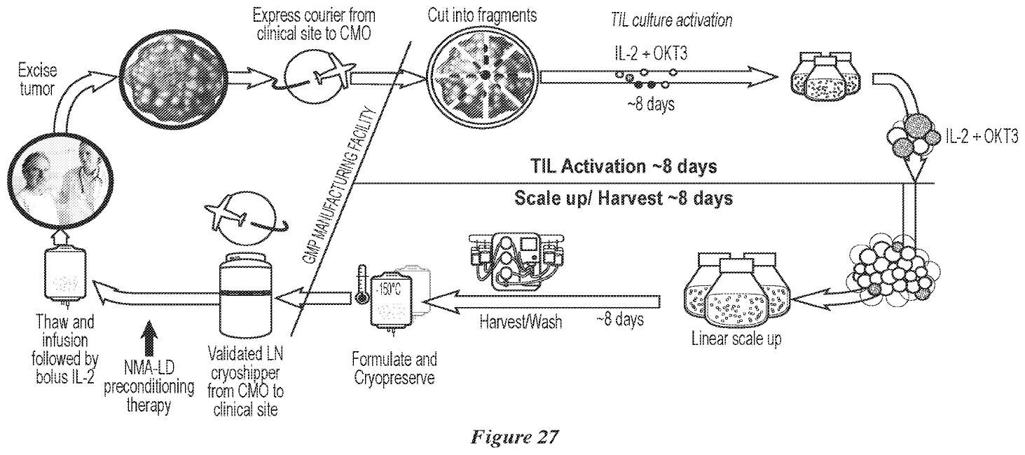

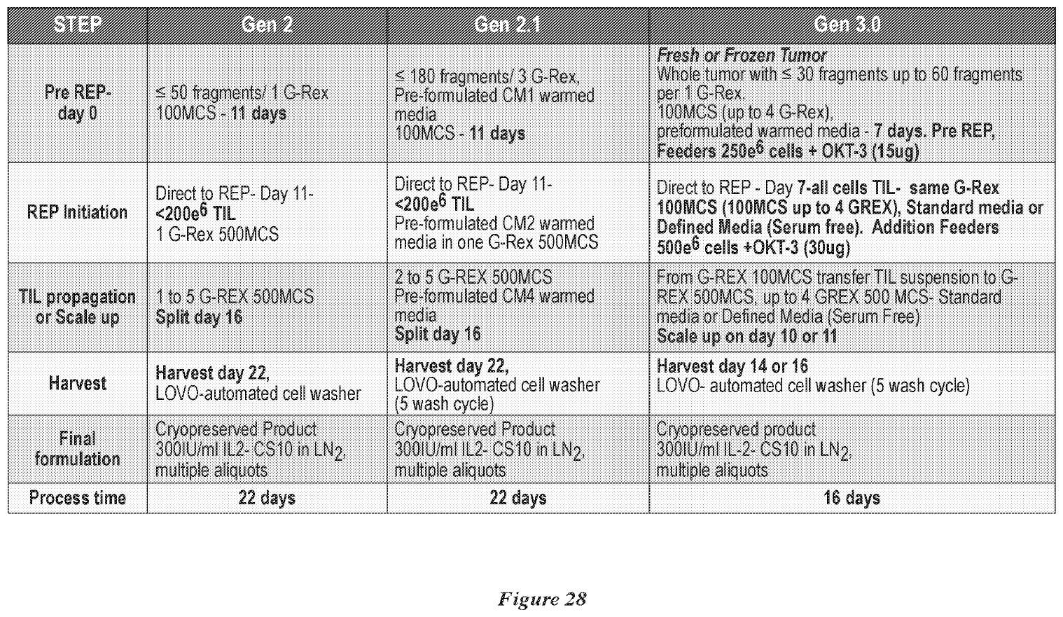

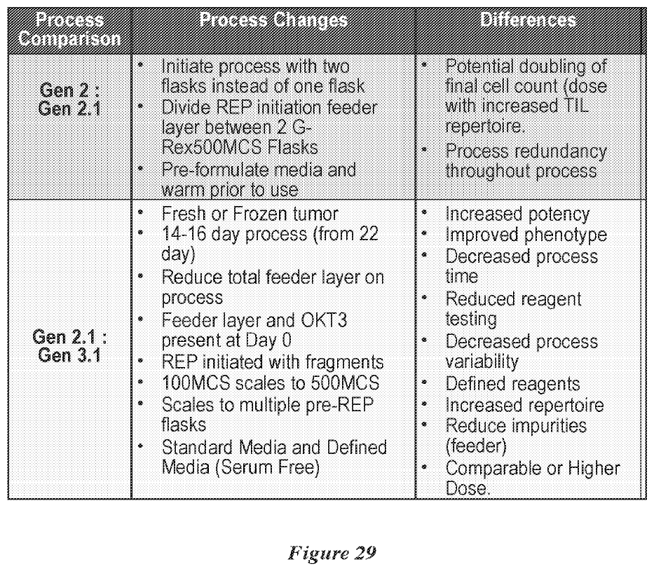

In some embodiments, the 4-1BB agonist is a 4-1BB agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof (see, FIG. 18). In structures I-A and I-B, the cylinders refer to individual polypeptide binding domains. Structures I-A and I-B comprise three linearly-linked TNFRSF binding domains derived from e.g., 4-1BBL (4-1BB ligand, CD137 ligand (CD137L), or tumor necrosis factor superfamily member 9 (TNFSF9)) or an antibody that binds 4-1BB, which fold to form a trivalent protein, which is then linked to a second triavelent protein through IgG1-Fc (including C_{H}3 and C_{H}2 domains) is then used to link two of the trivalent proteins together through disulfide bonds (small elongated ovals), stabilizing the structure and providing an agonists capable of bringing together the intracellular signaling domains of the six receptors and signaling proteins to form a signaling complex. The TNFRSF binding domains denoted as cylinders may be scFv domains comprising, e.g., a V_{H} and a V_{L} chain connected by a linker that may comprise hydrophilic residues and Gly and Ser sequences for flexibility, as well as Glu and Lys for solubility. Any scFv domain design may be used, such as those described in de Marco, Microbial Cell Factories, 2011, 10, 44; Ahmad, et al., Clin. & Dev. Immunol. 2012, 980250; Monnier, et al., Antibodies, 2013, 2, 193-208; or in references incorporated elsewhere herein. Fusion protein structures of this form are described in U.S. Pat. Nos. 9,359,420, 9,340,599, 8,921,519, and 8,450,460, the disclosures of which are incorporated by reference herein.

Amino acid sequences for the other polypeptide domains of structure I-A given in FIG. 18 are found in Table 16. The Fc domain preferably comprises a complete constant domain (amino acids 17-230 of SEQ ID NO:62) the complete hinge domain (amino acids 1-16 of SEQ ID NO:62) or a portion of the hinge domain (e.g., amino acids 4-16 of SEQ ID NO:62). Preferred linkers for connecting a C-terminal Fc-antibody may be selected from the embodiments given in SEQ ID NO:63 to SEQ ID NO:72, including linkers suitable for fusion of additional polypeptides.

TABLE 16
Amino acid sequences for TNFRSF agonist fusion proteins, including 4-1BB
agonist fusion proteins, with C-terminal Fc-antibody fragment fusion
protein design (structure I-A).

| Identifier | Sequence (One-Letter Amino Acid Symbols) | |
|---|---|---|
| SEQ ID NO: 62 | KSCDKTHTCP PCPAPELLGG PSVFLFPPKP KDTLMISRTP EVTCVVVDVS HEDPEVKENW | 60 |
| Fc domain | YVDGVEVHNA KTKPREEQYN STYRVVSVLT VLHQDWLNGK EYKCKVSNKA LPAPIEKTIS | 120 |
| | KAKGQPREPQ VYTLPPSREE MTKNQVSLTC LVKGFYPSDI AVEWESNGQP ENNYKTTPPV | 180 |
| | LDSDGSFFLY SKLTVDKSRW QQGNVFSCSV MHEALHNHYT QKSLSLSPGK | 230 |
| SEQ ID NO: 63 | GGPGSSKSCD KTHTCPPCPA PE | 22 |
| linker | | |
| SEQ ID NO: 64 | GGSGSSKSCD KTHTCPPCPA PE | 22 |
| linker | | |
| SEQ ID NO: 65 | GGPGSSSSSS SKSCDKTHTC PPCPAPE | 27 |
| linker | | |
| SEQ ID NO: 66 | GGSGSSSSSS SKSCDKTHTC PPCPAPE | 27 |
| linker | | |
| SEQ ID NO: 67 | GGPGSSSSSS SSSKSCDKTH TCPPCPAPE | 29 |
| linker | | |
| SEQ ID NO: 68 | GGSGSSSSSS SSSKSCDKTH TCPPCPAPE | 29 |
| linker | | |
| SEQ ID NO: 69 | GGPGSSGSGS SDKTHTCPPC PAPE | 24 |
| linker | | |
| SEQ ID NO: 70 | GGPGSSGSGS DKTHTCPPCP APE | 23 |
| linker | | |
| SEQ ID NO: 71 | GGPSSSGSDK THTCPPCPAP E | 21 |
| linker | | |
| SEQ ID NO: 72 | GGSSSSSSSS GSDKTHTCPP CPAPE | 25 |
| linker | | |

Amino acid sequences for the other polypeptide domains of structure I-B given in FIG. 18 are found in Table 17. If an Fc antibody fragment is fused to the N-terminus of an TNRFSF fusion protein as in structure I-B, the sequence of the Fc module is preferably that shown in SEQ ID NO: 73, and the linker sequences are preferably selected from those embodiments set forth in SEQ ID NO: 74 to SEQ ID NO: 76.

TABLE 17
Amino acid sequences for TNFRSF agonist fusion proteins, including 4-1BB
agonist fusion proteins, with N-terminal Fc-antibody fragment fusion
protein design (structure I-B).

| Identifier | Sequence (One-Letter Amino Acid Symbols) | |
|---|---|---|
| SEQ ID NO: 73 | METDTLLLWV LLLWVPAGNG DKTHTCPPCP APELLGGPSV FLFPPKPKDT LMISRTPEVT | 60 |
| Fc domain | CVVVDVSHED PEVKFNWYVD GVEVHNAKTK PREEQYNSTY RVVSVLTVLH QDWLNGKEYK | 120 |
| | CKVSNKALPA PIEKTISKAK GQPREPQVYT LPPSREEMTK NQVSLTCLVK GFYPSDIAVE | 180 |
| | WESNGQPENN YKTTPPVLDS DGSFFLYSKL TVDKSRWQQG NVFSCSVMHE ALHNHYTQKS | 240 |
| | LSLSPG | 246 |
| SEQ ID NO: 74 | SGSGSGSGSG S | 11 |
| linker | | |
| SEQ ID NO: 75 | SSSSSSGSGS GS | 12 |
| linker | | |
| SEQ ID NO: 76 | SSSSSSGSGS GSGSGS | 16 |
| linker | | |

In some embodiments, a 4-1BB agonist fusion protein according to structures I-A or I-B comprises one or more 4-1BB binding domains selected from the group consisting of a variable heavy chain and variable light chain of utomilumab, a variable heavy chain and variable light chain of urelumab, a variable heavy chain and variable light chain of utomilumab, a variable heavy chain and variable light chain selected from the variable heavy chains and variable light chains described in Table 18, any combination of a variable heavy chain and variable light chain of the foregoing, and fragments, derivatives, conjugates, variants, and biosimilars thereof.

In some embodiments, a 4-1BB agonist fusion protein according to structures I-A or I-B comprises one or more 4-1BB binding domains comprising a 4-1BBL sequence. In some embodiments, a 4-1BB agonist fusion protein according to structures I-A or I-B comprises one or more 4-1BB binding domains comprising a sequence according to SEQ ID NO:77. In some embodiments, a 4-1BB agonist fusion protein according to structures I-A or I-B comprises one or more 4-1BB binding domains comprising a soluble 4-1BBL sequence. In some embodiments, a 4-1BB agonist fusion protein according to structures I-A or I-B comprises one or more 4-1BB binding domains comprising a sequence according to SEQ ID NO:78.

In some embodiments, a 4-1BB agonist fusion protein according to structures I-A or I-B comprises one or more 4-1BB binding domains that is a scFv domain comprising V_{H} and V_{L} regions that are each at least 95% identical to the sequences shown in SEQ ID NO:44 and SEQ ID NO:45, respectively, wherein the V_{H} and V_{L} domains are connected by a linker. In some embodiments, a 4-1BB agonist fusion protein according to structures I-A or I-B comprises one or more 4-1BB binding domains that is a scFv domain comprising V_{H} and V_{L} regions that are each at least 95% identical to the sequences shown in SEQ ID NO:54 and SEQ ID NO:55, respectively, wherein the V_{H} and V_{L} domains are connected by a linker. In some embodiments, a 4-1BB agonist fusion protein according to structures I-A or I-B comprises one or more 4-1BB binding domains that is a scFv domain comprising V_{H} and V_{L} regions that are each at least 95% identical to the V_{H} and V_{L} sequences given in Table 18, wherein the V_{H} and V_{L} domains are connected by a linker.

TABLE 18
Additional polypeptide domains useful as 4-1BB binding domains in fusion
proteins or as scFv 4-1BB agonist antibodies.

| Identifier | Sequence (One-Letter Amino Acid Symbols) | |
|---|---|---|
| SEQ ID NO: 77 | MEYASDASLD PEAPWPPAPR ARACRVLPWA LVAGLLLLLL LAAACAVFLA CPWAVSGARA | 60 |
| 4 - 1BBL | SPGSAASPRL REGPELSPDD PAGLLDLRQG MFAQLVAQNV LLIDGPLSWY SDPGLAGVSL | 120 |
| | TGGLSYKEDT KELVVAKAGV YYVFFQLELR RVVAGEGSGS VSLALHLQPL RSAAGAAALA | 180 |
| | LTVDLPPASS EARNSAFGFQ GRLLHLSAGQ RLGVHLHTEA RARHAWQLTQ GATVLGLFRV | 240 |
| | TPEIPAGLPS PRSE | 254 |
| SEQ ID NO: 78 | LRQGMFAQLV AQNVLLIDGP LSWYSDPGLA GVSLTGGLSY KEDTKELVVA KAGVYYVFFQ | 60 |
| 4-1BBL soluble | LELRRVVAGE GSGSVSLALH LQPLRSAAGA AALALTVDLP PASSEARNSA FGFQGRLLHL | 120 |
| domain | SAGQRLGVHL HTEARARHAW QLTQGATVLG LFRVTPEIPA GLPSPRSE | 168 |
| SEQ ID NO: 79 | QVQLQQPGAE LVKPGASVKL SCKASGYTFS SYWMHWVKQR PGQVLEWIGE INPGNGHTNY | 60 |
| variable heavy | NEKFKSKATL TVDKSSSTAY MQLSSLTSED SAVYYCARSF TTARGFAYWG QGTLVTVS | 118 |
| chain for 4B4-1- | | |
| 1 version 1 | | |
| SEQ ID NO: 80 | DIVMTQSPAT QSVTPGDRVS LSCRASQTIS DYLHWYQQKS HESPRLLIKY ASQSISGIPS | 60 |
| variable light | RFSGSGSGSD FTLSINSVEP EDVGVYYCQD GHSFPPTFGG GTKLEIK | 107 |
| chain for 4B4-1- | | |
| 1 version 1 | | |
| SEQ ID NO: 81 | QVQLQQPGAE LVKPGASVKL SCKASGYTFS SYWMHWVKQR PGQVLEWIGE INPGNGHTNY | 60 |
| variable heavy | NEKFKSKATL TVDKSSSTAY MQLSSLTSED SAVYYCARSF TTARGFAYWG QGTLVTVSA | 119 |
| chain for 4B4-1- | | |
| 1 version 2 | | |
| SEQ ID NO: 82 | DIVMTQSPAT QSVTPGDRVS LSCRASQTIS DYLHWYQQKS HESPRLLIKY ASQSISGIPS | 60 |
| variable light | RFSGSGSGSD FTLSINSVEP EDVGVYYCQD GHSFPPTFGG GTKLEIKR | 108 |
| chain for 4B4-1- | | |
| 1 version 2 | | |
| SEQ ID NO: 83 | MDWTWRILFL VAAATGAHSE VQLVESGGGL VQPGGSLRLS CAASGFTFSD YWMSWVRQAP | 60 |
| variable heavy | GKGLEWVADI KNDGSYTNYA PSLTNRFTIS RDNAKNSLYL QMNSLRAEDT AVYYCARELT | 120 |
| chain for H39E3- | | |
| 2 | | |
| SEQ ID NO: 84 | MEAPAQLLFL LLLWLPDTTG DIVMTQSPDS LAVSLGERAT INCKSSQSLL SSGNQKNYL | 60 |
| variable light | WYQQKPGQPP KLLIYYASTR QSGVPDRFSG SGSGTDFTLT ISSLQAEDVA | 110 |
| chain for H39E3- | | |
| 2 | | |

In some embodiments, the 4-1BB agonist is a 4-1BB agonistic single-chain fusion polypeptide comprising (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, and wherein the additional domain is a Fab or Fc fragment domain. In some embodiments, the 4-1BB agonist is a 4-1BB agonistic single-chain fusion polypeptide comprising (i) a first soluble 4-1BB binding domain, (ii) a first peptide linker, (iii) a second soluble 4-1BB binding domain, (iv) a second peptide linker, and (v) a third soluble 4-1BB binding domain, further comprising an additional domain at the N-terminal and/or C-terminal end, wherein the additional domain is a Fab or Fc fragment domain, wherein each of the soluble 4-1BB domains lacks a stalk region (which contributes to trimerization and provides a certain distance to the cell membrane, but is not part of the 4-1BB binding domain) and the first and the second peptide linkers independently have a length of 3-8 amino acids.

In some embodiments, the 4-1BB agonist is a 4-1BB agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein each TNF superfamily cytokine domain is a 4-1BB binding domain.

In some embodiments, the 4-1BB agonist is a 4-1BB agonistic scFv antibody comprising any of the foregoing V_{H} domains linked to any of the foregoing V_{L} domains.

In some embodiments, the 4-1BB agonist is BPS Bioscience 4-1BB agonist antibody catalog no. 79097-2, commercially available from BPS Bioscience, San Diego, CA, USA. In some embodiments, the 4-1BB agonist is Creative Biolabs 4-1BB agonist antibody catalog no. MOM-18179, commercially available from Creative Biolabs, Shirley, NY, USA.

### 3. OX40 (CD134) Agonists

In some embodiments, the TNFRSF agonist is an OX40 (CD134) agonist. The OX40 agonist may be any OX40 binding molecule known in the art. The OX40 binding molecule may be a monoclonal antibody or fusion protein capable of binding to human or mammalian OX40. The OX40 agonists or OX40 binding molecules may comprise an immunoglobulin heavy chain of any isotype (e.g., IgG, IgE, IgM, IgD, IgA, and IgY), class (e.g., IgG1, IgG2, IgG3, IgG4, IgA1 and IgA2) or subclass of immunoglobulin molecule. The OX40 agonist or OX40 binding molecule may have both a heavy and a light chain. As used herein, the term binding molecule also includes antibodies (including full length antibodies), monoclonal antibodies (including full length monoclonal antibodies), polyclonal antibodies, multispecific antibodies (e.g., bispecific antibodies), human, humanized or chimeric antibodies, and antibody fragments, e.g., Fab fragments, F(ab′) fragments, fragments produced by a Fab expression library, epitope-binding fragments of any of the above, and engineered forms of antibodies, e.g., scFv molecules, that bind to OX40. In some embodiments, the OX40 agonist is an antigen binding protein that is a fully human antibody. In some embodiments, the OX40 agonist is an antigen binding protein that is a humanized antibody. In some embodiments, OX40 agonists for use in the presently disclosed methods and compositions include anti-OX40 antibodies, human anti-OX40 antibodies, mouse anti-OX40 antibodies, mammalian anti-OX40 antibodies, monoclonal anti-OX40 antibodies, polyclonal anti-OX40 antibodies, chimeric anti-OX40 antibodies, anti-OX40 adnectins, anti-OX40 domain antibodies, single chain anti-OX40 fragments, heavy chain anti-OX40 fragments, light chain anti-OX40 fragments, anti-OX40 fusion proteins, and fragments, derivatives, conjugates, variants, or biosimilars thereof. In some embodiments, the OX40 agonist is an agonistic, anti-OX40 humanized or fully human monoclonal antibody (i.e., an antibody derived from a single cell line).

In some embodiments, the OX40 agonist or OX40 binding molecule may also be a fusion protein. OX40 fusion proteins comprising an Fc domain fused to OX40L are described, for example, in Sadun, et al., J. Immunother. 2009, 182, 1481-89. In some embodiments, a multimeric OX40 agonist, such as a trimeric or hexameric OX40 agonist (with three or six ligand binding domains), may induce superior receptor (OX40L) clustering and internal cellular signaling complex formation compared to an agonistic monoclonal antibody, which typically possesses two ligand binding domains. Trimeric (trivalent) or hexameric (or hexavalent) or greater fusion proteins comprising three TNFRSF binding domains and IgG1-Fc and optionally further linking two or more of these fusion proteins are described, e.g., in Gieffers, et al., Mol. Cancer Therapeutics 2013, 12, 2735-47. Agonistic OX40 antibodies and fusion proteins are known to induce strong immune responses. Curti, et al., Cancer Res. 2013, 73, 7189-98. In some embodiments, the OX40 agonist is a monoclonal antibody or fusion protein that binds specifically to OX40 antigen in a manner sufficient to reduce toxicity. In some embodiments, the OX40 agonist is an agonistic OX40 monoclonal antibody or fusion protein that abrogates antibody-dependent cellular toxicity (ADCC), for example NK cell cytotoxicity. In some embodiments, the OX40 agonist is an agonistic OX40 monoclonal antibody or fusion protein that abrogates antibody-dependent cell phagocytosis (ADCP). In some embodiments, the OX40 agonist is an agonistic OX40 monoclonal antibody or fusion protein that abrogates complement-dependent cytotoxicity (CDC). In some embodiments, the OX40 agonist is an agonistic OX40 monoclonal antibody or fusion protein which abrogates Fc region functionality.

In some embodiments, the OX40 agonists are characterized by binding to human OX40 (SEQ ID NO:85) with high affinity and agonistic activity. In some embodiments, the OX40 agonist is a binding molecule that binds to human OX40 (SEQ ID NO:85). In some embodiments, the OX40 agonist is a binding molecule that binds to murine OX40 (SEQ ID NO:86). The amino acid sequences of OX40 antigen to which an OX40 agonist or binding molecule binds are summarized in Table 19.

TABLE 19
Amino acid sequences of OX40 antigens.

| Identifier | Sequence (One-Letter Amino Acid Symbols) | |
|---|---|---|
| SEQ ID NO: 85 | MCVGARRIGR GPCAALLLLG LGLSTVTGLH CVGDTYPSND RCCHECRPGN GMVSRCSRSQ | 60 |
| human OX40 | NTVCRPCGPG FYNDVVSSKP CKPCTWCNLR SGSERKQLCT ATQDTVCRCR AGTQPLDSYK | 120 |
| (Homo sapiens) | PGVDCAPCPP GHFSPGDNQA CKPWTNCTLA GKHTLQPASN SSDAICEDRD PPATQPQETQ | 180 |
| | GPPARPITVQ PTEAWPRTSQ GPSTRPVEVP GGRAVAAILG LGLVLGLLGP LAILLALYLL | 240 |
| | RRDQRLPPDA HKPPGGGSFR TPIQEEQADA HSTLAKI | 277 |
| SEQ ID NO: 86 | MYVWVQQPTA LLLLGLTLGV TARRLNCVKH TYPSGHKCCR ECQPGHGMVS RCDHTRDTLC | 60 |
| murine OX40 | HPCETGFYNE AVNYDTCKQC TQCNHRSGSE LKQNCTPTQD TVCRCRPGTQ PRQDSGYKLG | 120 |
| (Mus musculus) | VDCVPCPPGH FSPGNNQACK PWTNCTLSGK QTRHPASDSL DAVCEDRSLL ATLLWETQRP | 180 |
| | TFRPTTVQST TVWPRTSELP SPPTLVTPEG PAFAVLLGLG LGLLAPLTVL LALYLLRKAW | 240 |
| | RLPNTPKPCW GNSFRTPIQE EHTDAHFTLA KI | 272 |

In some embodiments, the compositions, processes and methods described include a OX40 agonist that binds human or murine OX40 with a K_{D} of about 100 pM or lower, binds human or murine OX40 with a K_{D} of about 90 pM or lower, binds human or murine OX40 with a K_{D} of about 80 pM or lower, binds human or murine OX40 with a K_{D} of about 70 pM or lower, binds human or murine OX40 with a K_{D} of about 60 pM or lower, binds human or murine OX40 with a K_{D} of about 50 pM or lower, binds human or murine OX40 with a K_{D} of about 40 pM or lower, or binds human or murine OX40 with a K_{D} of about 30 pM or lower.

In some embodiments, the compositions, processes and methods described include a OX40 agonist that binds to human or murine OX40 with a k_{assoc} of about 7.5×10⁵ l/M·s or faster, binds to human or murine OX40 with a k_{assoc} of about 7.5×10⁵ l/M·s or faster, binds to human or murine OX40 with a k_{assoc} of about 8×10⁵ l/M·s or faster, binds to human or murine OX40 with a k_{assoc} of about 8.5×10⁵ l/M·s or faster, binds to human or murine OX40 with a k_{assoc} of about 9×10⁵ l/M·s or faster, binds to human or murine OX40 with a k_{assoc} of about 9.5×10⁵ l/M·s or faster, or binds to human or murine OX40 with a k_{assoc} of about 1×10⁶ l/M·s or faster.

In some embodiments, the compositions, processes and methods described include a OX40 agonist that binds to human or murine OX40 with a k_{dissoc} of about 2×10⁻⁵ l/s or slower, binds to human or murine OX40 with a k_{dissoc} of about 2.1×10⁻⁵ l/s or slower, binds to human or murine OX40 with a k_{dissoc} of about 2.2×10⁻⁵ l/s or slower, binds to human or murine OX40 with a k_{dissoc} of about 2.3×10⁻⁵ l/s or slower, binds to human or murine OX40 with a k_{dissoc} of about 2.4×10⁻⁵ l/s or slower, binds to human or murine OX40 with a k_{dissoc} of about 2.5×10⁻⁵ l/s or slower, binds to human or murine OX40 with a k_{dissoc} of about 2.6×10⁻⁵ l/s or slower or binds to human or murine OX40 with a k_{dissoc} of about 2.7×10⁻⁵ l/s or slower, binds to human or murine OX40 with a k_{dissoc} of about 2.8×10⁻⁵ l/s or slower, binds to human or murine OX40 with a k_{dissoc} of about 2.9×10⁻⁵ l/s or slower, or binds to human or murine OX40 with a k_{dissoc} of about 3×10⁻⁵ l/s or slower.

In some embodiments, the compositions, processes and methods described include OX40 agonist that binds to human or murine OX40 with an IC₅₀ of about 10 nM or lower, binds to human or murine OX40 with an IC₅₀ of about 9 nM or lower, binds to human or murine OX40 with an IC₅₀ of about 8 nM or lower, binds to human or murine OX40 with an IC₅₀ of about 7 nM or lower, binds to human or murine OX40 with an IC₅₀ of about 6 nM or lower, binds to human or murine OX40 with an IC₅₀ of about 5 nM or lower, binds to human or murine OX40 with an IC₅₀ of about 4 nM or lower, binds to human or murine OX40 with an IC₅₀ of about 3 nM or lower, binds to human or murine OX40 with an IC₅₀ of about 2 nM or lower, or binds to human or murine OX40 with an IC₅₀ of about 1 nM or lower.

In some embodiments, the OX40 agonist is tavolixizumab, also known as MEDI0562 or MEDI-0562. Tavolixizumab is available from the MedImmune subsidiary of AstraZeneca, Inc. Tavolixizumab is immunoglobulin Gi-kappa, anti-[Homo sapiens TNFRSF4 (tumor necrosis factor receptor (TNFR) superfamily member 4, OX40, CD134)], humanized and chimeric monoclonal antibody. The amino acid sequences of tavolixizumab are set forth in Table 20. Tavolixizumab comprises N-glycosylation sites at positions 301 and 301″, with fucosylated complex bi-antennary CHO-type glycans; heavy chain intrachain disulfide bridges at positions 22-95 (V_{H}-V_{L}), 148-204 (C_{H}1-C_{L}), 265-325 (C_{H}2) and 371-429 (C_{H}3) (and at positions 22″-95″, 148″-204″, 265″-325″, and 371″-429″); light chain intrachain disulfide bridges at positions 23′-88′ (V_{H}-V_{L}) and 134′-194′ (C_{H}1-C_{L}) (and at positions 23′″-88′″ and 134′″-194′″); interchain heavy chain-heavy chain disulfide bridges at positions 230-230″ and 233-233″; and interchain heavy chain-light chain disulfide bridges at 224-214′ and 224″-214′″. Current clinical trials of tavolixizumab in a variety of solid tumor indications include U.S. National Institutes of Health clinicaltrials.gov identifiers NCT02318394 and NCT02705482.

In some embodiments, a OX40 agonist comprises a heavy chain given by SEQ ID NO:87 and a light chain given by SEQ ID NO:88. In some embodiments, a OX40 agonist comprises heavy and light chains having the sequences shown in SEQ ID NO:87 and SEQ ID NO:88, respectively, or antigen binding fragments, Fab fragments, single-chain variable fragments (scFv), variants, or conjugates thereof. In some embodiments, a OX40 agonist comprises heavy and light chains that are each at least 99% identical to the sequences shown in SEQ ID NO:87 and SEQ ID NO:88, respectively. In some embodiments, a OX40 agonist comprises heavy and light chains that are each at least 98% identical to the sequences shown in SEQ ID NO:87 and SEQ ID NO:88, respectively. In some embodiments, a OX40 agonist comprises heavy and light chains that are each at least 97% identical to the sequences shown in SEQ ID NO:87 and SEQ ID NO:88, respectively. In some embodiments, a OX40 agonist comprises heavy and light chains that are each at least 96% identical to the sequences shown in SEQ ID NO:87 and SEQ ID NO:88, respectively. In some embodiments, a OX40 agonist comprises heavy and light chains that are each at least 95% identical to the sequences shown in SEQ ID NO:87 and SEQ ID NO:88, respectively.

In some embodiments, the OX40 agonist comprises the heavy and light chain CDRs or variable regions (VRs) of tavolixizumab. In some embodiments, the OX40 agonist heavy chain variable region (V_{H}) comprises the sequence shown in SEQ ID NO:89, and the OX40 agonist light chain variable region (V_{L}) comprises the sequence shown in SEQ ID NO:90, and conservative amino acid substitutions thereof. In some embodiments, a OX40 agonist comprises V_{H} and V_{L} regions that are each at least 99% identical to the sequences shown in SEQ ID NO:89 and SEQ ID NO:90, respectively. In some embodiments, a OX40 agonist comprises V_{H} and V_{L} regions that are each at least 98% identical to the sequences shown in SEQ ID NO:89 and SEQ ID NO:90, respectively. In some embodiments, a OX40 agonist comprises V_{H} and V_{L} regions that are each at least 97% identical to the sequences shown in SEQ ID NO:89 and SEQ ID NO:90, respectively. In some embodiments, a OX40 agonist comprises V_{H} and V_{L} regions that are each at least 96% identical to the sequences shown in SEQ ID NO:89 and SEQ ID NO:90, respectively. In some embodiments, a OX40 agonist comprises V_{H} and V_{L} regions that are each at least 95% identical to the sequences shown in SEQ ID NO:89 and SEQ ID NO:90, respectively. In some embodiments, an OX40 agonist comprises an scFv antibody comprising V_{H} and V_{L} regions that are each at least 99% identical to the sequences shown in SEQ ID NO:89 and SEQ ID NO:90.

In some embodiments, a OX40 agonist comprises heavy chain CDR1, CDR2 and CDR3 domains having the sequences set forth in SEQ ID NO:91, SEQ ID NO:92, and SEQ ID NO:93, respectively, and conservative amino acid substitutions thereof, and light chain CDR1, CDR2 and CDR3 domains having the sequences set forth in SEQ ID NO:94, SEQ ID NO:95, and SEQ ID NO:96, respectively, and conservative amino acid substitutions thereof.

In some embodiments, the OX40 agonist is a OX40 agonist biosimilar monoclonal antibody approved by drug regulatory authorities with reference to tavolixizumab. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is tavolixizumab. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is tavolixizumab. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tavolixizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tavolixizumab.

TABLE 20
Amino acid sequences for OX40 agonist antibodies related to tavolixizumab.

| Identifier | Sequence (One-Letter Amino Acid Symbols) | |
|---|---|---|
| SEQ ID NO: 87 | QVQLQESGPG LVKPSQTLSL TCAVYGGSFS SGYWNWIRKH PGKGLEYIGY ISYNGITYHN | 60 |
| heavy chain for | PSLKSRITIN RDTSKNQYSL QLNSVTPEDT AVYYCARYKY DYDGGHAMDY WGQGTLVTVS | 120 |
| tavolixizumab | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDKTHTC PPCPAPELLG | 240 |
| | GPSVFLFPPK PKDTLMISRT PEVTCVVVDV SHEDPEVKEN WYVDGVEVHN AKTKPREEQY | 300 |
| | NSTYRVVSVL TVLHQDWING KEYKCKVSNK ALPAPIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 88 | DIQMTQSPSS LSASVGDRVT ITCRASQDIS NYLNWYQQKP GKAPKLLIYY TSKLHSGVPS | 60 |
| light chain for | RFSGSGSGTD YTLTISSLQP EDFATYYCQQ GSALPWTFGQ GTKVEIKRTV AAPSVFIFPP | 120 |
| tavolixizumab | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSEN RGEC | 214 |
| SEQ ID NO: 89 | QVQLQESGPG LVKPSQTLSL TCAVYGGSFS SGYWNWIRKH PGKGLEYIGY ISYNGITYHN | 60 |
| heavy chain | PSLKSRITIN RDTSKNQYSL QLNSVTPEDT AVYYCARYKY DYDGGHAMDY WGQGTLVT | 118 |
| variable region | | |
| for | | |
| tavolixizumab | | |
| SEQ ID NO: 90 | DIQMTQSPSS LSASVGDRVT ITCRASQDIS NYLNWYQQKP GKAPKLLIYY TSKLHSGVPS | 60 |
| light chain | RFSGSGSGTD YTLTISSLQP EDFATYYCQQ GSALPWTFGQ GTKVEIKR | 108 |
| variable region | | |
| for | | |
| tavolixizumab | | |
| SEQ ID NO: 91 | GSFSSGYWN | 9 |
| heavy chain CDR1 | | |
| for | | |
| tavolixizumab | | |
| SEQ ID NO: 92 | YIGYISYNGI TYH | 13 |
| heavy chain CDR2 | | |
| for | | |
| tavolixizumab | | |
| SEQ ID NO: 93 | RYKYDYDGGH AMDY | 14 |
| heavy chain CDR3 | | |
| for | | |
| tavolixizumab | | |
| SEQ ID NO: 94 | QDISNYLN | 8 |
| light chain CDR1 | | |
| for | | |
| tavolixizumab | | |
| SEQ ID NO: 95 | LLIYYTSKLH S | 11 |
| light chain CDR2 | | |
| for | | |
| tavolixizumab | | |
| SEQ ID NO: 96 | QQGSALPW | 8 |
| light chain CDR3 | | |
| for | | |
| tavolixizumab | | |

In some embodiments, the OX40 agonist is 11D4, which is a fully human antibody available from Pfizer, Inc. The preparation and properties of 11D4 are described in U.S. Pat. Nos. 7,960,515; 8,236,930; and 9,028,824, the disclosures of which are incorporated by reference herein. The amino acid sequences of 11D4 are set forth in Table 21.

In some embodiments, a OX40 agonist comprises a heavy chain given by SEQ ID NO:97 and a light chain given by SEQ ID NO:98. In some embodiments, a OX40 agonist comprises heavy and light chains having the sequences shown in SEQ ID NO:97 and SEQ ID NO:98, respectively, or antigen binding fragments, Fab fragments, single-chain variable fragments (scFv), variants, or conjugates thereof. In some embodiments, a OX40 agonist comprises heavy and light chains that are each at least 99% identical to the sequences shown in SEQ ID NO:97 and SEQ ID NO:98, respectively. In some embodiments, a OX40 agonist comprises heavy and light chains that are each at least 98% identical to the sequences shown in SEQ ID NO:97 and SEQ ID NO:98, respectively. In some embodiments, a OX40 agonist comprises heavy and light chains that are each at least 97% identical to the sequences shown in SEQ ID NO:97 and SEQ ID NO:98, respectively. In some embodiments, a OX40 agonist comprises heavy and light chains that are each at least 96% identical to the sequences shown in SEQ ID NO:97 and SEQ ID NO:98, respectively. In some embodiments, a OX40 agonist comprises heavy and light chains that are each at least 95% identical to the sequences shown in SEQ ID NO:97 and SEQ ID NO:98, respectively.

In some embodiments, the OX40 agonist comprises the heavy and light chain CDRs or variable regions (VRs) of 11D4. In some embodiments, the OX40 agonist heavy chain variable region (V_{H}) comprises the sequence shown in SEQ ID NO:99, and the OX40 agonist light chain variable region (V_{L}) comprises the sequence shown in SEQ ID NO: 100, and conservative amino acid substitutions thereof. In some embodiments, a OX40 agonist comprises V_{H} and V_{L} regions that are each at least 99% identical to the sequences shown in SEQ ID NO:99 and SEQ ID NO:100, respectively. In some embodiments, a OX40 agonist comprises V_{H} and V_{L} regions that are each at least 98% identical to the sequences shown in SEQ ID NO:99 and SEQ ID NO:100, respectively. In some embodiments, a OX40 agonist comprises V_{H} and V_{L} regions that are each at least 97% identical to the sequences shown in SEQ ID NO:99 and SEQ ID NO:100, respectively. In some embodiments, a OX40 agonist comprises V_{H} and V_{L} regions that are each at least 96% identical to the sequences shown in SEQ ID NO:99 and SEQ ID NO:100, respectively. In some embodiments, a OX40 agonist comprises V_{H} and V_{L} regions that are each at least 95% identical to the sequences shown in SEQ ID NO:99 and SEQ ID NO:100, respectively.

In some embodiments, a OX40 agonist comprises heavy chain CDR1, CDR2 and CDR3 domains having the sequences set forth in SEQ ID NO:101, SEQ ID NO:102, and SEQ ID NO:103, respectively, and conservative amino acid substitutions thereof, and light chain CDR1, CDR2 and CDR3 domains having the sequences set forth in SEQ ID NO:104, SEQ ID NO:105, and SEQ ID NO:106, respectively, and conservative amino acid substitutions thereof.

In some embodiments, the OX40 agonist is a OX40 agonist biosimilar monoclonal antibody approved by drug regulatory authorities with reference to 11D4. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 11D4. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is 11D4. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 11D4. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 11D4.

TABLE 21
Amino acid sequences for OX40 agonist antibodies related to 11D4.

| Identifier | Sequence (One-Letter Amino Acid Symbols) | |
|---|---|---|
| SEQ ID NO: 97 | EVQLVESGGG LVQPGGSLRL SCAASGFTFS SYSMNWVRQA PGKGLEWVSY ISSSSSTIDY | 60 |
| heavy chain for | ADSVKGRFTI SRDNAKNSLY LQMNSLRDED TAVYYCARES GWYLFDYWGQ GTLVTVSSAS | 120 |
| 11D4 | TKGPSVFPLA PCSRSTSEST AALGCLVKDY FPEPVTVSWN SGALTSGVHT FPAVLQSSGL | 180 |
| | YSLSSVVTVP SSNFGTQTYT CNVDHKPSNT KVDKTVERKC CVECPPCPAP PVAGPSVFLF | 240 |
| | PPKPKDTLMI SRTPEVTCVV VDVSHEDPEV QFNWYVDGVE VHNAKTKPRE EQFNSTFRVV | 300 |
| | SVLTVVHQDW LNGKEYKCKV SNKGLPAPIE KTISKTKGQP REPQVYTLPP SREEMTKNQV | 360 |
| | SLTCLVKGFY PSDIAVEWES NGQPENNYKT TPPMLDSDGS FFLYSKLTVD KSRWQQGNVF | 420 |
| | SCSVMHEALH NHYTQKSLSL SPGK | 444 |
| SEQ ID NO: 98 | DIQMTQSPSS LSASVGDRVT ITCRASQGIS SWLAWYQQKP EKAPKSLIYA ASSLQSGVPS | 60 |
| light chain for | RFSGSGSGTD FTLTISSLQP EDFATYYCQQ YNSYPPTFGG GTKVEIKRTV AAPSVFIFPP | 120 |
| 11D4 | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSEN RGEC | 214 |
| SEQ ID NO: 99 | EVQLVESGGG LVQPGGSLRL SCAASGFTFS SYSMNWVRQA PGKGLEWVSY ISSSSSTIDY | 60 |
| heavy chain | ADSVKGRFTI SRDNAKNSLY LQMNSLRDED TAVYYCARES GWYLFDYWGQ GTLVTVSS | 118 |
| variable region | | |
| for 11D4 | | |
| SEQ ID NO: 100 | DIQMTQSPSS LSASVGDRVT ITCRASQGIS SWLAWYQQKP EKAPKSLIYA ASSLQSGVPS | 60 |
| light chain | RFSGSGSGTD FTLTISSLQP EDFATYYCQQ YNSYPPTFGG GTKVEIK | 107 |
| variable region | | |
| for 11D4 | | |
| SEQ ID NO: 101 | SYSMN | 5 |
| heavy chain CDR1 | | |
| for 11D4 | | |
| SEQ ID NO: 102 | YISSSSSTID YADSVKG | 17 |
| heavy chain CDR2 | | |
| for 11D4 | | |
| SEQ ID NO: 103 | ESGWYLFDY | 9 |
| heavy chain CDR3 | | |
| for 11D4 | | |
| SEQ ID NO: 104 | RASQGISSWL A | 11 |
| light chain CDR1 | | |
| for 11D4 | | |
| SEQ ID NO: 105 | AASSLQS | 7 |
| light chain CDR2 | | |
| for 11D4 | | |
| SEQ ID NO: 106 | QQYNSYPPT | 9 |
| light chain CDR3 | | |
| for 11D4 | | |

In some embodiments, the OX40 agonist is 18D8, which is a fully human antibody available from Pfizer, Inc. The preparation and properties of 18D8 are described in U.S. Pat. Nos. 7,960,515; 8,236,930; and 9,028,824, the disclosures of which are incorporated by reference herein. The amino acid sequences of 18D8 are set forth in Table 22.

In some embodiments, a OX40 agonist comprises a heavy chain given by SEQ ID NO: 107 and a light chain given by SEQ ID NO: 108. In some embodiments, a OX40 agonist comprises heavy and light chains having the sequences shown in SEQ ID NO: 107 and SEQ ID NO: 108, respectively, or antigen binding fragments, Fab fragments, single-chain variable fragments (scFv), variants, or conjugates thereof. In some embodiments, a OX40 agonist comprises heavy and light chains that are each at least 99% identical to the sequences shown in SEQ ID NO: 107 and SEQ ID NO: 108, respectively. In some embodiments, a OX40 agonist comprises heavy and light chains that are each at least 98% identical to the sequences shown in SEQ ID NO: 107 and SEQ ID NO: 108, respectively. In some embodiments, a OX40 agonist comprises heavy and light chains that are each at least 97% identical to the sequences shown in SEQ ID NO:107 and SEQ ID NO:108, respectively. In some embodiments, a OX40 agonist comprises heavy and light chains that are each at least 96% identical to the sequences shown in SEQ ID NO:107 and SEQ ID NO:108, respectively. In some embodiments, a OX40 agonist comprises heavy and light chains that are each at least 95% identical to the sequences shown in SEQ ID NO:107 and SEQ ID NO:108, respectively.

In some embodiments, the OX40 agonist comprises the heavy and light chain CDRs or variable regions (VRs) of 18D8. In some embodiments, the OX40 agonist heavy chain variable region (V_{H}) comprises the sequence shown in SEQ ID NO:109, and the OX40 agonist light chain variable region (V_{L}) comprises the sequence shown in SEQ ID NO:110, and conservative amino acid substitutions thereof. In some embodiments, a OX40 agonist comprises V_{H} and V_{L} regions that are each at least 99% identical to the sequences shown in SEQ ID NO:109 and SEQ ID NO:110, respectively. In some embodiments, a OX40 agonist comprises V_{H} and V_{L} regions that are each at least 98% identical to the sequences shown in SEQ ID NO:109 and SEQ ID NO:110, respectively. In some embodiments, a OX40 agonist comprises V_{H} and V_{L} regions that are each at least 97% identical to the sequences shown in SEQ ID NO:109 and SEQ ID NO:110, respectively. In some embodiments, a OX40 agonist comprises V_{H} and V_{L} regions that are each at least 96% identical to the sequences shown in SEQ ID NO:109 and SEQ ID NO:110, respectively. In some embodiments, a OX40 agonist comprises V_{H} and V_{L} regions that are each at least 95% identical to the sequences shown in SEQ ID NO:109 and SEQ ID NO:110, respectively.

In some embodiments, a OX40 agonist comprises heavy chain CDR1, CDR2 and CDR3 domains having the sequences set forth in SEQ ID NO:111, SEQ ID NO:112, and SEQ ID NO:113, respectively, and conservative amino acid substitutions thereof, and light chain CDR1, CDR2 and CDR3 domains having the sequences set forth in SEQ ID NO:114, SEQ ID NO:115, and SEQ ID NO:116, respectively, and conservative amino acid substitutions thereof.

In some embodiments, the OX40 agonist is a OX40 agonist biosimilar monoclonal antibody approved by drug regulatory authorities with reference to 18D8. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is 18D8. In some embodiments, the one or more post-translational modifications are selected from one or more of glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is 18D8. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 18D8. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is 18D8.

TABLE 22
Amino acid sequences for OX40 agonist antibodies related to 18D8.

| Identifier | Sequence (One-Letter Amino Acid Symbols) | |
|---|---|---|
| SEQ ID NO: 107 | EVQLVESGGG LVQPGRSLRL SCAASGFTFD DYAMHWVRQA PGKGLEWVSG ISWNSGSIGY | 60 |
| heavy chain for | ADSVKGRFTI SRDNAKNSLY LQMNSLRAED TALYYCAKDQ STADYYFYYG MDVWGQGTTV | 120 |
| 18D8 | TVSSASTKGP SVFPLAPCSR STSESTAALG CLVKDYFPEP VTVSWNSGAL TSGVHTFPAV | 180 |
| | LQSSGLYSLS SVVTVPSSNF GTQTYTCNVD HKPSNTKVDK TVERKCCVEC PPCPAPPVAG | 240 |
| | PSVFLFPPKP KDTLMISRTP EVTCVVVDVS HEDPEVQFNW YVDGVEVHNA KTKPREEQFN | 300 |
| | STFRVVSVLT VVHQDWLNGK EYKCKVSNKG LPAPIEKTIS KTKGQPREPQ VYTLPPSREE | 360 |
| | MTKNQVSLTC LVKGFYPSDI AVEWESNGQP ENNYKTTPPM LDSDGSFFLY SKLTVDKSRW | 420 |
| | QQGNVFSCSV MHEALHNHYT QKSLSLSPGK | 450 |
| SEQ ID NO: 108 | EIVVTQSPAT LSLSPGERAT LSCRASQSVS SYLAWYQQKP GQAPRLLIYD ASNRATGIPA | 60 |
| light chain for | RFSGSGSGTD FTLTISSLEP EDFAVYYCQQ RSNWPTFGQG TKVEIKRTVA APSVFIFPPS | 120 |
| 18D8 | DEQLKSGTAS VVCLLNNFYP REAKVQWKVD NALQSGNSQE SVTEQDSKDS TYSLSSTLTL | 180 |
| | SKADYEKHKV YACEVTHQGL SSPVTKSENR GEC | 213 |
| SEQ ID NO: 109 | EVQLVESGGG LVQPGRSLRL SCAASGFTFD DYAMHWVRQA PGKGLEWVSG ISWNSGSIGY | 60 |
| heavy chain | ADSVKGRFTI SRDNAKNSLY LQMNSLRAED TALYYCAKDQ STADYYFYYG MDVWGQGTTV | 120 |
| variable region | TVSS | 124 |
| for 18D8 | | |
| SEQ ID NO: 110 | EIVVTQSPAT LSLSPGERAT LSCRASQSVS SYLAWYQQKP GQAPRLLIYD ASNRATGIPA | 60 |
| light chain | | |
| variable region | RFSGSGSGTD FTLTISSLEP EDFAVYYCQQ RSNWPTFGQG TKVEIK | 106 |
| for 18D8 | | |
| SEQ ID NO: 111 | DYAMH | 5 |
| heavy chain CDR1 | | |
| for 18D8 | | |
| SEQ ID NO: 112 | GISWNSGSIG YADSVKG | 17 |
| heavy chain CDR2 | | |
| for 18D8 | | |
| SEQ ID NO: 113 | DQSTADYYFY YGMDV | 15 |
| heavy chain CDR3 | | |
| for 18D8 | | |
| SEQ ID NO: 114 | RASQSVSSYL A | 11 |
| light chain CDR1 | | |
| for 18D8 | | |
| SEQ ID NO: 115 | DASNRAT | 7 |
| light chain CDR2 | | |
| for 18D8 | | |
| SEQ ID NO: 116 | QQRSNWPT | 8 |
| light chain CDR3 | | |
| for 18D8 | | |

In some embodiments, the OX40 agonist is Hu119-122, which is a humanized antibody available from GlaxoSmithKline plc. The preparation and properties of Hu119-122 are described in U.S. Pat. Nos. 9,006,399 and 9,163,085, and in International Patent Publication No. WO 2012/027328, the disclosures of which are incorporated by reference herein. The amino acid sequences of Hu119-122 are set forth in Table 23.

In some embodiments, the OX40 agonist comprises the heavy and light chain CDRs or variable regions (VRs) of Hu119-122. In some embodiments, the OX40 agonist heavy chain variable region (V_{H}) comprises the sequence shown in SEQ ID NO:117, and the OX40 agonist light chain variable region (V_{L}) comprises the sequence shown in SEQ ID NO:118, and conservative amino acid substitutions thereof. In some embodiments, a OX40 agonist comprises V_{H} and V_{L} regions that are each at least 99% identical to the sequences shown in SEQ ID NO:117 and SEQ ID NO:118, respectively. In some embodiments, a OX40 agonist comprises V_{H} and V_{L} regions that are each at least 98% identical to the sequences shown in SEQ ID NO:117 and SEQ ID NO:118, respectively. In some embodiments, a OX40 agonist comprises V_{H} and V_{L} regions that are each at least 97% identical to the sequences shown in SEQ ID NO:117 and SEQ ID NO:118, respectively. In some embodiments, a OX40 agonist comprises V_{H} and V_{L} regions that are each at least 96% identical to the sequences shown in SEQ ID NO:117 and SEQ ID NO:118, respectively. In some embodiments, a OX40 agonist comprises V_{H} and V_{L} regions that are each at least 95% identical to the sequences shown in SEQ ID NO:117 and SEQ ID NO:118, respectively.

In some embodiments, a OX40 agonist comprises heavy chain CDR1, CDR2 and CDR3 domains having the sequences set forth in SEQ ID NO:119, SEQ ID NO:120, and SEQ ID NO:121, respectively, and conservative amino acid substitutions thereof, and light chain CDR1, CDR2 and CDR3 domains having the sequences set forth in SEQ ID NO:122, SEQ ID NO:123, and SEQ ID NO:124, respectively, and conservative amino acid substitutions thereof.

In some embodiments, the OX40 agonist is a OX40 agonist biosimilar monoclonal antibody approved by drug regulatory authorities with reference to Hu119-122. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is Hu119-122. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is Hu119-122. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu119-122. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu119-122.

TABLE 23
Amino acid sequences for OX40 agonist antibodies related to Hul19-122.

| Identifier | Sequence (One-Letter Amino Acid Symbols) | |
|---|---|---|
| SEQ ID NO: 117 | EVQLVESGGG LVQPGGSLRL SCAASEYEFP SHDMSWVRQA PGKGLELVAA INSDGGSTYY | 60 |
| heavy chain | PDTMERRFTI SRDNAKNSLY LQMNSLRAED TAVYYCARHY DDYYAWFAYW GQGTMVTVSS | 120 |
| variable region | | |
| for Hu119-122 | | |
| SEQ ID NO: 118 | EIVLTQSPAT LSLSPGERAT LSCRASKSVS TSGYSYMHWY QQKPGQAPRL LIYLASNLES | 60 |
| light chain | GVPARFSGSG SGTDFTLTIS SLEPEDFAVY YCQHSRELPL TFGGGTKVEI K | 111 |
| variable region | | |
| for Hu119-122 | | |
| SEQ ID NO: 119 | SHDMS | 5 |
| heavy chain CDR1 | | |
| for Hu119-122 | | |
| SEQ ID NO: 120 | AINSDGGSTY YPDTMER | 17 |
| heavy chain CDR2 | | |
| for Hu119-122 | | |
| SEQ ID NO: 121 | HYDDYYAWFA Y | 11 |
| heavy chain CDR3 | | |
| for Hu119-122 | | |
| SEQ ID NO: 122 | RASKSVSTSG YSYMH | 15 |
| light chain CDR1 | | |
| for Hu119-122 | | |
| SEQ ID NO: 123 | LASNLES | 7 |
| light chain CDR2 | | |
| for Hu119-122 | | |
| SEQ ID NO: 124 | QHSRELPLT | 9 |
| light chain CDR3 | | |
| for Hu119-122 | | |

In some embodiments, the OX40 agonist is Hu106-222, which is a humanized antibody available from GlaxoSmithKline plc. The preparation and properties of Hu106-222 are described in U.S. Pat. Nos. 9,006,399 and 9,163,085, and in International Patent Publication No. WO 2012/027328, the disclosures of which are incorporated by reference herein. The amino acid sequences of Hu106-222 are set forth in Table 24.

In some embodiments, the OX40 agonist comprises the heavy and light chain CDRs or variable regions (VRs) of Hu106-222. In some embodiments, the OX40 agonist heavy chain variable region (V_{H}) comprises the sequence shown in SEQ ID NO:125, and the OX40 agonist light chain variable region (V_{L}) comprises the sequence shown in SEQ ID NO:126, and conservative amino acid substitutions thereof. In some embodiments, a OX40 agonist comprises V_{H} and V_{L} regions that are each at least 99% identical to the sequences shown in SEQ ID NO:125 and SEQ ID NO:126, respectively. In some embodiments, a OX40 agonist comprises V_{H} and V_{L} regions that are each at least 98% identical to the sequences shown in SEQ ID NO:125 and SEQ ID NO:126, respectively. In some embodiments, a OX40 agonist comprises V_{H} and V_{L} regions that are each at least 97% identical to the sequences shown in SEQ ID NO:125 and SEQ ID NO:126, respectively. In some embodiments, a OX40 agonist comprises V_{H} and V_{L} regions that are each at least 96% identical to the sequences shown in SEQ ID NO:125 and SEQ ID NO:126, respectively. In some embodiments, a OX40 agonist comprises V_{H} and V_{L} regions that are each at least 95% identical to the sequences shown in SEQ ID NO:125 and SEQ ID NO:126, respectively.

In some embodiments, a OX40 agonist comprises heavy chain CDR1, CDR2 and CDR3 domains having the sequences set forth in SEQ ID NO:127, SEQ ID NO:128, and SEQ ID NO:129, respectively, and conservative amino acid substitutions thereof, and light chain CDR1, CDR2 and CDR3 domains having the sequences set forth in SEQ ID NO:130, SEQ ID NO:131, and SEQ ID NO:132, respectively, and conservative amino acid substitutions thereof.

In some embodiments, the OX40 agonist is a OX40 agonist biosimilar monoclonal antibody approved by drug regulatory authorities with reference to Hu106-222. In some embodiments, the biosimilar monoclonal antibody comprises an OX40 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is Hu106-222. In some embodiments, the one or more post-translational modifications are selected from one or more of: glycosylation, oxidation, deamidation, and truncation. In some embodiments, the biosimilar is a OX40 agonist antibody authorized or submitted for authorization, wherein the OX40 agonist antibody is provided in a formulation which differs from the formulations of a reference medicinal product or reference biological product, wherein the reference medicinal product or reference biological product is Hu106-222. The OX40 agonist antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu106-222. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is Hu106-222.

TABLE 24
Amino acid sequences for OX40 agonist antibodies related to Hul06-222.

| Identifier | Sequence (One-Letter Amino Acid Symbols) | |
|---|---|---|
| SEQ ID NO: 125 | QVQLVQSGSE LKKPGASVKV SCKASGYTFT DYSMHWVRQA PGQGLKWMGW INTETGEPTY | 60 |
| heavy chain | ADDFKGRFVF SLDTSVSTAY LQISSLKAED TAVYYCANPY YDYVSYYAMD YWGQGTTVTV | 120 |
| variable region | SS | 122 |
| for Hu106-222 | | |
| SEQ ID NO: 126 | DIQMTQSPSS LSASVGDRVT ITCKASQDVS TAVAWYQQKP GKAPKLLIYS ASYLYTGVPS | 60 |
| light chain | RFSGSGSGTD FTFTISSLQP EDIATYYCQQ HYSTPRTFGQ GTKLEIK | 107 |
| variable region | | |
| for Hu106-222 | | |
| SEQ ID NO: 127 | DYSMH | 5 |
| heavy chain CDR1 | | |
| for Hu106-222 | | |
| SEQ ID NO: 128 | WINTETGEPT YADDFKG | 17 |
| heavy chain CDR2 | | |
| for Hu106-222 | | |
| SEQ ID NO: 129 | PYYDYVSYYA MDY | 13 |
| heavy chain CDR3 | | |
| for Hu106-222 | | |
| SEQ ID NO: 130 | KASQDVSTAV A | 11 |
| light chain CDR1 | | |
| for Hu106-222 | | |
| SEQ ID NO: 131 | SASYLYT | 7 |
| light chain CDR2 | | |
| for Hu106-222 | | |
| SEQ ID NO: 132 | QQHYSTPRT | 9 |
| light chain CDR3 | | |
| for Hu106-222 | | |

In some embodiments, the OX40 agonist antibody is MEDI6469 (also referred to as 9B12). MEDI6469 is a murine monoclonal antibody. Weinberg, et al., J Immunother. 2006, 29, 575-585. In some embodiments the OX40 agonist is an antibody produced by the 9B12 hybridoma, deposited with Biovest Inc. (Malvern, MA, USA), as described in Weinberg, et al., J Immunother. 2006, 29, 575-585, the disclosure of which is hereby incorporated by reference in its entirety. In some embodiments, the antibody comprises the CDR sequences of MEDI6469. In some embodiments, the antibody comprises a heavy chain variable region sequence and/or a light chain variable region sequence of MEDI6469.

In some embodiments, the OX40 agonist is L106 BD (Pharmingen Product #340420). In some embodiments, the OX40 agonist comprises the CDRs of antibody L106 (BD Pharmingen Product #340420). In some embodiments, the OX40 agonist comprises a heavy chain variable region sequence and/or a light chain variable region sequence of antibody L106 (BD Pharmingen Product #340420). In some embodiments, the OX40 agonist is ACT35 (Santa Cruz Biotechnology, Catalog #20073). In some embodiments, the OX40 agonist comprises the CDRs of antibody ACT35 (Santa Cruz Biotechnology, Catalog #20073). In some embodiments, the OX40 agonist comprises a heavy chain variable region sequence and/or a light chain variable region sequence of antibody ACT35 (Santa Cruz Biotechnology, Catalog #20073). In some embodiments, the OX40 agonist is the murine monoclonal antibody anti-mCD134/mOX40 (clone OX86), commercially available from InVivoMAb, BioXcell Inc, West Lebanon, NH.

In some embodiments, the OX40 agonist is selected from the OX40 agonists described in International Patent Application Publication Nos. WO 95/12673, WO 95/21925, WO 2006/121810, WO 2012/027328, WO 2013/028231, WO 2013/038191, and WO 2014/148895; European Patent Application EP 0672141; U.S. Patent Application Publication Nos. US 2010/136030, US 2014/377284, US 2015/190506, and US 2015/132288 (including clones 20E5 and 12H3); and U.S. Pat. Nos. 7,504,101, 7,550,140, 7,622,444, 7,696,175, 7,960,515, 7,961,515, 8,133,983, 9,006,399, and 9,163,085, the disclosure of each of which is incorporated herein by reference in its entirety.

In some embodiments, the OX40 agonist is an OX40 agonistic fusion protein as depicted in Structure I-A (C-terminal Fc-antibody fragment fusion protein) or Structure I-B (N-terminal Fc-antibody fragment fusion protein), or a fragment, derivative, conjugate, variant, or biosimilar thereof. The properties of structures I-A and I-B are described above and in U.S. Pat. Nos. 9,359,420, 9,340,599, 8,921,519, and 8,450,460, the disclosures of which are incorporated by reference herein. Amino acid sequences for the polypeptide domains of structure I-A given in FIG. 18 are found in Table 16. The Fc domain preferably comprises a complete constant domain (amino acids 17-230 of SEQ ID NO:62) the complete hinge domain (amino acids 1-16 of SEQ ID NO:62) or a portion of the hinge domain (e.g., amino acids 4-16 of SEQ ID NO:62). Preferred linkers for connecting a C-terminal Fc-antibody may be selected from the embodiments given in SEQ ID NO:63 to SEQ ID NO:72, including linkers suitable for fusion of additional polypeptides. Likewise, amino acid sequences for the polypeptide domains of structure I-B given in FIG. 18 are found in Table 17. If an Fc antibody fragment is fused to the N-terminus of an TNRFSF fusion protein as in structure I-B, the sequence of the Fc module is preferably that shown in SEQ ID NO:73, and the linker sequences are preferably selected from those embodiments set forth in SEQ ID NO:74 to SEQ ID NO:76.

In some embodiments, an OX40 agonist fusion protein according to structures I-A or I-B comprises one or more OX40 binding domains selected from the group consisting of a variable heavy chain and variable light chain of tavolixizumab, a variable heavy chain and variable light chain of 11D4, a variable heavy chain and variable light chain of 18D8, a variable heavy chain and variable light chain of Hu119-122, a variable heavy chain and variable light chain of Hu106-222, a variable heavy chain and variable light chain selected from the variable heavy chains and variable light chains described in Table 25, any combination of a variable heavy chain and variable light chain of the foregoing, and fragments, derivatives, conjugates, variants, and biosimilars thereof.

In some embodiments, an OX40 agonist fusion protein according to structures I-A or I-B comprises one or more OX40 binding domains comprising an OX40L sequence. In some embodiments, an OX40 agonist fusion protein according to structures I-A or I-B comprises one or more OX40 binding domains comprising a sequence according to SEQ ID NO:133. In some embodiments, an OX40 agonist fusion protein according to structures I-A or I-B comprises one or more OX40 binding domains comprising a soluble OX40L sequence. In some embodiments, a OX40 agonist fusion protein according to structures I-A or I-B comprises one or more OX40 binding domains comprising a sequence according to SEQ ID NO:134. In some embodiments, a OX40 agonist fusion protein according to structures I-A or I-B comprises one or more OX40 binding domains comprising a sequence according to SEQ ID NO:135.

In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:89 and SEQ ID NO:90, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:99 and SEQ ID NO:100, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:109 and SEQ ID NO:110, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:127 and SEQ ID NO:128, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the sequences shown in SEQ ID NO:125 and SEQ ID NO:126, respectively, wherein the V_{H} and V_{L} domains are connected by a linker. In some embodiments, an OX40 agonist fusion protein according to structures I-A or I-B comprises one or more OX40 binding domains that is a scFv domain comprising V_{H} and V_{L} regions that are each at least 95% identical to the V_{H} and V_{L} sequences given in Table 25, wherein the V_{H} and V_{L} domains are connected by a linker.

TABLE 25
Additional polypeptide domains useful as OX40 binding domains in fusion
proteins (e.g., structures I-A and I-B) or as scFv OX40 agonist antibodies.

| Identifier | Sequence (One-Letter Amino Acid Symbols) | |
|---|---|---|
| SEQ ID NO: 133 | MERVQPLEEN VGNAARPRFE RNKLLLVASV IQGLGLLLCF TYICLHFSAL QVSHRYPRIQ | 60 |
| OX40L | SIKVQFTEYK KEKGFILTSQ KEDEIMKVQN NSVIINCDGF YLISLKGYFS QEVNISLHYQ | 120 |
| | KDEEPLFQLK KVRSVNSLMV ASLTYKDKVY LNVTTDNTSL DDFHVNGGEL ILIHQNPGEF | 180 |
| | CVL | 183 |
| SEQ ID NO: 134 | SHRYPRIQSI KVQFTEYKKE KGFILTSQKE DEIMKVQNNS VIINCDGFYL ISLKGYFSQE | 60 |
| OX40L soluble | VNISLHYQKD EEPLFQLKKV RSVNSLMVAS LTYKDKVYLN VTTDNTSLDD FHVNGGELIL | 120 |
| domain | IHQNPGEFCV L | 131 |
| SEQ ID NO: 135 | YPRIQSIKVQ FTEYKKEKGF ILTSQKEDEI MKVQNNSVII NCDGFYLISL KGYFSQEVNI | 60 |
| OX40L soluble | SLHYQKDEEP LFQLKKVRSV NSLMVASLTY KDKVYLNVTT DNTSLDDFHV NGGELILIHQ | 120 |
| domain | NPGEFCVL | 128 |
| (alternative) | | |
| SEQ ID NO: 136 | EVQLVESGGG LVQPGGSLRL SCAASGFTFS NYTMNWVRQA PGKGLEWVSA ISGSGGSTYY | 60 |
| variable heavy | ADSVKGRFTI SRDNSKNTLY LQMNSLRAED TAVYYCAKDR YSQVHYALDY WGQGTLVTVS | 120 |
| chain for 008 | | |
| SEQ ID NO: 137 | DIVMTQSPDS LPVTPGEPAS ISCRSSQSLL HSNGYNYLDW YLQKAGQSPQ LLIYLGSNRA | 60 |
| variable light | SGVPDRFSGS GSGTDFTLKI SRVEAEDVGV YYCQQYYNHP TTFGQGTK | 108 |
| chain for 008 | | |
| SEQ ID NO: 138 | EVQLVESGGG VVQPGRSLRL SCAASGFTFS DYTMNWVRQA PGKGLEWVSS ISGGSTYYAD | 60 |
| variable heavy | SRKGRFTISR DNSKNTLYLQ MNNLRAEDTA VYYCARDRYF RQQNAFDYWG QGTLVTVSSA | 120 |
| chain for 011 | | |
| SEQ ID NO: 139 | DIVMTQSPDS LPVTPGEPAS ISCRSSQSLL HSNGYNYLDW YLQKAGQSPQ LLIYLGSNRA | 60 |
| variable light | SGVPDRFSGS GSGTDFTLKI SRVEAEDVGV YYCQQYYNHP TTFGQGTK | 108 |
| chain for 011 | | |
| SEQ ID NO: 140 | EVQLVESGGG LVQPRGSLRL SCAASGFTFS SYAMNWVRQA PGKGLEWVAV ISYDGSNKYY | 60 |
| variable heavy | ADSVKGRFTI SRDNSKNTLY LQMNSLRAED TAVYYCAKDR YITLPNALDY WGQGTLVTVS | 120 |
| chain for 021 | | |
| SEQ ID NO: 141 | DIQMTQSPVS LPVTPGEPAS ISCRSSQSLL HSNGYNYLDW YLQKPGQSPQ LLIYLGSNRA | 60 |
| variable light | SGVPDRFSGS GSGTDFTLKI SRVEAEDVGV YYCQQYKSNP PTFGQGTK | 108 |
| chain for 021 | | |
| SEQ ID NO: 142 | EVQLVESGGG LVHPGGSLRL SCAGSGFTFS SYAMHWVRQA PGKGLEWVSA IGTGGGTYYA | 60 |
| variable heavy | DSVMGRFTIS RDNSKNTLYL QMNSLRAEDT AVYYCARYDN VMGLYWFDYW GQGTLVTVSS | 120 |
| chain for 023 | | |
| SEQ ID NO: 143 | EIVLTQSPAT LSLSPGERAT LSCRASQSVS SYLAWYQQKP GQAPRLLIYD ASNRATGIPA | 60 |
| variable light | RFSGSGSGTD FTLTISSLEP EDFAVYYCQQ RSNWPPAFGG GTKVEIKR | 108 |
| chain for 023 | | |
| SEQ ID NO: 144 | EVQLQQSGPE LVKPGASVKM SCKASGYTFT SYVMHWVKQK PGQGLEWIGY INPYNDGTKY | 60 |
| heavy chain | NEKFKGKATL TSDKSSSTAY MELSSLTSED SAVYYCANYY GSSLSMDYWG QGTSVTVSS | 119 |
| variable region | | |
| SEQ ID NO: 145 | DIQMTQTTSS LSASLGDRVT ISCRASQDIS NYLNWYQQKP DGTVKLLIYY TSRLHSGVPS | 60 |
| light chain | RFSGSGSGTD YSLTISNLEQ EDIATYFCQQ GNTLPWTFGG GTKLEIKR | 108 |
| variable region | | |
| SEQ ID NO: 146 | EVQLQQSGPE LVKPGASVKI SCKTSGYTFK DYTMHWVKQS HGKSLEWIGG IYPNNGGSTY | 60 |
| heavy chain | NQNFKDKATL TVDKSSSTAY MEFRSLTSED SAVYYCARMG YHGPHLDFDV WGAGTTVTVS | 120 |
| variable region | P | 121 |
| SEQ ID NO: 147 | DIVMTQSHKF MSTSLGDRVS ITCKASQDVG AAVAWYQQKP GQSPKLLIYW ASTRHTGVPD | 60 |
| light chain | RFTGGGSGTD FTLTISNVQS EDLTDYFCQQ YINYPLTFGG GTKLEIKR | 108 |
| variable region | | |
| SEQ ID NO: 148 | QIQLVQSGPE LKKPGETVKI SCKASGYTFT DYSMHWVKQA PGKGLKWMGW INTETGEPTY | 60 |
| heavy chain | ADDFKGRFAF SLETSASTAY LQINNLKNED TATYFCANPY YDYVSYYAMD YWGHGTSVTV | 120 |
| variable region | SS | 122 |
| of humanized | | |
| antibody | | |
| SEQ ID NO: 149 | QVQLVQSGSE LKKPGASVKV SCKASGYTFT DYSMHWVRQA PGQGLKWMGW INTETGEPTY | 60 |
| heavy chain | ADDFKGRFVF SLDTSVSTAY LQISSLKAED TAVYYCANPY YDYVSYYAMD YWGQGTTVTV | 120 |
| variable region | SS | 122 |
| of humanized | | |
| antibody | | |
| SEQ ID NO: 150 | DIVMTQSHKF MSTSVRDRVS ITCKASQDVS TAVAWYQQKP GQSPKLLIYS ASYLYTGVPD | 60 |
| light chain | RFTGSGSGTD FTFTISSVQA EDLAVYYCQQ HYSTPRTFGG GTKLEIK | 107 |
| variable region | | |
| of humanized | | |
| antibody | | |
| SEQ ID NO: 151 | DIVMTQSHKF MSTSVRDRVS ITCKASQDVS TAVAWYQQKP GQSPKLLIYS ASYLYTGVPD | 60 |
| light chain | RFTGSGSGTD FTFTISSVQA EDLAVYYCQQ HYSTPRTFGG GTKLEIK | 107 |
| variable region | | |
| of humanized | | |
| antibody | | |
| SEQ ID NO: 152 | EVQLVESGGG LVQPGESLKL SCESNEYEFP SHDMSWVRKT PEKRLELVAA INSDGGSTYY | 60 |
| heavy chain | PDTMERRFII SRDNTKKTLY LQMSSLRSED TALYYCARHY DDYYAWFAYW GQGTLVTVSA | 120 |
| variable region | | |
| of humanized | | |
| antibody | | |
| SEQ ID NO: 153 | EVQLVESGGG LVQPGGSLRL SCAASEYEFP SHDMSWVRQA PGKGLELVAA INSDGGSTYY | 60 |
| heavy chain | PDTMERRFTI SRDNAKNSLY LQMNSLRAED TAVYYCARHY DDYYAWFAYW GQGTMVTVSS | 120 |
| variable region | | |
| of humanized | | |
| antibody | | |
| SEQ ID NO: 154 | DIVLTQSPAS LAVSLGQRAT ISCRASKSVS TSGYSYMHWY QQKPGQPPKL LIYLASNLES | 60 |
| light chain | GVPARFSGSG SGTDFTLNIH PVEEEDAATY YCQHSRELPL TFGAGTKLEL K | 111 |
| variable region | | |
| of humanized | | |
| antibody | | |
| SEQ ID NO: 155 | EIVLTQSPAT LSLSPGERAT LSCRASKSVS TSGYSYMHWY QQKPGQAPRL LIYLASNLES | 60 |
| light chain | GVPARFSGSG SGTDFTLTIS SLEPEDFAVY YCQHSRELPL TFGGGTKVEI K | 111 |
| variable region | | |
| of humanized | | |
| antibody | | |
| SEQ ID NO: 156 | MYLGLNYVFI VFLLNGVQSE VKLEESGGGL VQPGGSMKLS CAASGFTFSD AWMDWVRQSP | 60 |
| heavy chain | EKGLEWVAEI RSKANNHATY YAESVNGRFT ISRDDSKSSV YLQMNSLRAE DTGIYYCTWG | 120 |
| variable region | EVFYFDYWGQ GTTLTVSS | 138 |
| SEQ ID NO: 157 | MRPSIQFLGL LLFWLHGAQC DIQMTQSPSS LSASLGGKVT ITCKSSQDIN KYIAWYQHKP | 60 |
| light chain | GKGPRLLIHY TSTLQPGIPS RFSGSGSGRD YSFSISNLEP EDIATYYCLQ YDNLLTFGAG | 120 |
| variable region | TKLELK | 126 |

In some embodiments, the OX40 agonist is a OX40 agonistic single-chain fusion polypeptide comprising (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, and wherein the additional domain is a Fab or Fc fragment domain. In some embodiments, the OX40 agonist is a OX40 agonistic single-chain fusion polypeptide comprising (i) a first soluble OX40 binding domain, (ii) a first peptide linker, (iii) a second soluble OX40 binding domain, (iv) a second peptide linker, and (v) a third soluble OX40 binding domain, further comprising an additional domain at the N-terminal and/or C-terminal end, wherein the additional domain is a Fab or Fc fragment domain wherein each of the soluble OX40 binding domains lacks a stalk region (which contributes to trimerisation and provides a certain distance to the cell membrane, but is not part of the OX40 binding domain) and the first and the second peptide linkers independently have a length of 3-8 amino acids.

In some embodiments, the OX40 agonist is an OX40 agonistic single-chain fusion polypeptide comprising (i) a first soluble tumor necrosis factor (TNF) superfamily cytokine domain, (ii) a first peptide linker, (iii) a second soluble TNF superfamily cytokine domain, (iv) a second peptide linker, and (v) a third soluble TNF superfamily cytokine domain, wherein each of the soluble TNF superfamily cytokine domains lacks a stalk region and the first and the second peptide linkers independently have a length of 3-8 amino acids, and wherein the TNF superfamily cytokine domain is an OX40 binding domain.

In some embodiments, the OX40 agonist is MEDI6383. MEDI6383 is an OX40 agonistic fusion protein and can be prepared as described in U.S. Pat. No. 6,312,700, the disclosure of which is incorporated by reference herein.

In some embodiments, the OX40 agonist is an OX40 agonistic scFv antibody comprising any of the foregoing V_{H} domains linked to any of the foregoing V_{L} domains.

In some embodiments, the OX40 agonist is Creative Biolabs OX40 agonist monoclonal antibody MOM-18455, commercially available from Creative Biolabs, Inc., Shirley, NY, USA.

In some embodiments, the OX40 agonist is OX40 agonistic antibody clone Ber-ACT35 commercially available from BioLegend, Inc., San Diego, CA, USA.

### B. Optional Cell Viability Analyses

Optionally, a cell viability assay can be performed after the priming first expansion (sometimes referred to as the initial bulk expansion), using standard assays known in the art. Thus, in certain embodiments, the method comprises performing a cell viability assay subsequent to the priming first expansion. For example, a trypan blue exclusion assay can be done on a sample of the bulk TILs, which selectively labels dead cells and allows a viability assessment. Other assays for use in testing viability can include but are not limited to the Alamar blue assay; and the MTT assay.

### 1. Cell Counts, Viability, Flow Cytometry

In some embodiments, cell counts and/or viability are measured. The expression of markers such as but not limited CD3, CD4, CD8, and CD56, as well as any other disclosed or described herein, can be measured by flow cytometry with antibodies, for example but not limited to those commercially available from BD Bio-sciences (BD Biosciences, San Jose, CA) using a FACSCanto™ flow cytometer (BD Biosciences). The cells can be counted manually using a disposable c-chip hemocytometer (VWR, Batavia, IL) and viability can be assessed using any method known in the art, including but not limited to trypan blue staining. The cell viability can also be assayed based on U.S. Patent Application Publication No. 2018/0282694, incorporated by reference herein in its entirety. Cell viability can also be assayed based on U.S. Patent Application Publication No. 2018/0280436 or International Patent Application Publication No. WO/2018/081473, both of which are incorporate herein in their entireties for all purposes.

In some cases, the bulk TIL population can be cryopreserved immediately, using the protocols discussed below. Alternatively, the bulk TIL population can be subjected to REP and then cryopreserved as discussed below. Similarly, in the case where genetically modified TILs will be used in therapy, the bulk or REP TIL populations can be subjected to genetic modifications for suitable treatments.

### 2. Cell Cultures

In some embodiments, a method for expanding TILs, including those discussed above as well as exemplified in FIGS. 1 and 8, in particular, e.g., FIG. 8A and/or FIG. 8B and/or FIG. 8C and/or FIG. 8D, may include using about 5,000 mL to about 25,000 mL of cell medium, about 5,000 mL to about 10,000 mL of cell medium, or about 5,800 mL to about 8,700 mL of cell medium. In some embodiments, the media is a serum free medium. In some embodiments, the media in the priming first expansion is serum free. In some embodiments, the media in the second expansion is serum free. In some embodiments, the media in the priming first expansion and the second expansion (also referred to as rapid second expansion) are both serum free. In some embodiments, expanding the number of TILs uses no more than one type of cell culture medium. Any suitable cell culture medium may be used, e.g., AIM-V cell medium (L-glutamine, 50 μM streptomycin sulfate, and 10 μM gentamicin sulfate) cell culture medium (Invitrogen, Carlsbad CA). In this regard, the inventive methods advantageously reduce the amount of medium and the number of types of medium required to expand the number of TIL. In some embodiments, expanding the number of TIL may comprise feeding the cells no more frequently than every third or fourth day. Expanding the number of cells in a gas permeable container simplifies the procedures necessary to expand the number of cells by reducing the feeding frequency necessary to expand the cells.

In some embodiments, the cell culture medium in the first and/or second gas permeable container is unfiltered. The use of unfiltered cell medium may simplify the procedures necessary to expand the number of cells. In some embodiments, the cell medium in the first and/or second gas permeable container lacks beta-mercaptoethanol (BME).

In some embodiments, the duration of the method comprising obtaining a tumor tissue sample from the mammal; culturing the tumor tissue sample in a first gas permeable container containing cell medium including IL-2, 1× antigen-presenting feeder cells, and OKT-3 for a duration of about 1 to 8 days, e.g., about 7 days as a priming first expansion, or about 8 days as a priming first expansion; transferring the TILs to a second gas permeable container and expanding the number of TILs in the second gas permeable container containing cell medium including IL-2, 2× antigen-presenting feeder cells, and OKT-3 for a duration of about 7 to 9 days, e.g., about 7 days, about 8 days, or about 9 days.

In some embodiments, the duration of the method comprising obtaining a tumor tissue sample from the mammal; culturing the tumor tissue sample in a first gas permeable container containing cell medium including IL-2, 1× antigen-presenting feeder cells, and OKT-3 for a duration of about 1 to 7 days, e.g., about 7 days as a priming first expansion; transferring the TILs to a second gas permeable container and expanding the number of TILs in the second gas permeable container containing cell medium including IL-2, 2× antigen-presenting feeder cells, and OKT-3 for a duration of about 7 to 14 days, or about 7 to 9 days, e.g., about 7 days, about 8 days, or about 9 days, about 10 days, or about 11 days.

In some embodiments, the duration of the method comprising obtaining a tumor tissue sample from the mammal; culturing the tumor tissue sample in a first gas permeable container containing cell medium including IL-2, 1× antigen-presenting feeder cells, and OKT-3 for a duration of about 1 to 7 days, e.g., about 7 days, as a priming first expansion; transferring the TILs to a second gas permeable container and expanding the number of TILs in the second gas permeable container containing cell medium including IL-2, 2× antigen-presenting feeder cells, and OKT-3 for a duration of about 7 to 11 days, e.g., about 7 days, about 8 days, about 9 days, about 10, or about 11 days.

In some embodiments, TILs are expanded in gas-permeable containers. Gas-permeable containers have been used to expand TILs using PBMCs using methods, compositions, and devices known in the art, including those described in U.S. Patent Application Publication No. 2005/0106717 A1, the disclosures of which are incorporated herein by reference. In some embodiments, TILs are expanded in gas-permeable bags. In some embodiments, TILs are expanded using a cell expansion system that expands TILs in gas permeable bags, such as the Xuri Cell Expansion System W25 (GE Healthcare). In some embodiments, TILs are expanded using a cell expansion system that expands TILs in gas permeable bags, such as the WAVE Bioreactor System, also known as the Xuri Cell Expansion System W5 (GE Healthcare). In some embodiments, the cell expansion system includes a gas permeable cell bag with a volume selected from the group consisting of about 100 mL, about 200 mL, about 300 mL, about 400 mL, about 500 mL, about 600 mL, about 700 mL, about 800 mL, about 900 mL, about 1 L, about 2 L, about 3 L, about 4 L, about 5 L, about 6 L, about 7 L, about 8 L, about 9 L, and about 10 L.

In some embodiments, TILs can be expanded in G-REX flasks (commercially available from Wilson Wolf Manufacturing). Such embodiments allow for cell populations to expand from about 5×10⁵ cells/cm² to between 10×10⁶ and 30×10⁶ cells/cm². In some embodiments this is without feeding. In some embodiments, this is without feeding so long as medium resides at a height of about 10 cm in the G-REX flask. In some embodiments this is without feeding but with the addition of one or more cytokines. In some embodiments, the cytokine can be added as a bolus without any need to mix the cytokine with the medium. Such containers, devices, and methods are known in the art and have been used to expand TILs, and include those described in U.S. Patent Application Publication No. US 2014/0377739A1, International Publication No. WO 2014/210036 A1, U.S. Patent Application Publication No. us 2013/0115617 A1, International Publication No. WO 2013/188427 A1, U.S. Patent Application Publication No. US 2011/0136228 A1, U.S. Pat. No. 8,809,050 B2, International publication No. WO 2011/072088 A2, U.S. Patent Application Publication No. US 2016/0208216 A1, U.S. Patent Application Publication No. US 2012/0244133 A1, International Publication No. WO 2012/129201 A1, U.S. Patent Application Publication No. US 2013/0102075 A1, U.S. Pat. No. 8,956,860 B2, International Publication No. WO 2013/173835 A1, U.S. Patent Application Publication No. US 2015/0175966 A1, the disclosures of which are incorporated herein by reference. Such processes are also described in Jin et al., J. Immunotherapy, 2012, 35:283-292.

### C. Optional Knockdown or Knockout of Genes in TILs

In some embodiments, the expanded TILs of the present invention are further manipulated before, during, or after an expansion step, including during closed, sterile manufacturing processes, each as provided herein, in order to alter protein expression in a transient manner. In some embodiments, the transiently altered protein expression is due to transient gene editing. In some embodiments, the expanded TILs of the present invention are treated with transcription factors (TFs) and/or other molecules capable of transiently altering protein expression in the TILs. In some embodiments, the TFs and/or other molecules that are capable of transiently altering protein expression provide for altered expression of tumor antigens and/or an alteration in the number of tumor antigen-specific T cells in a population of TILs.

In certain embodiments, the method comprises genetically editing a population of TILs. In certain embodiments, the method comprises genetically editing the first population of TILs, the second population of TILs and/or the third population of TILs.

In some embodiments, the present invention includes genetic editing through nucleotide insertion, such as through ribonucleic acid (RNA) insertion, including insertion of messenger RNA (mRNA), short hairpint RNA (shRNA), or small (or short) interfering RNA (siRNA), into a population of TILs for promotion of the expression of one or more proteins or inhibition of the expression of one or more proteins, as well as simultaneous combinations of both promotion of one set of proteins with inhibition of another set of proteins.

In some embodiments, the expanded TILs of the present invention undergo transient alteration of protein expression. In some embodiments, the transient alteration of protein expression occurs in the bulk TIL population prior to first expansion, including, for example in the TIL population obtained from for example, Step A as indicated in FIG. 8 (particularly FIG. 8A and/or FIG. 8B and/or FIG. 8C and/or FIG. 8D). In some embodiments, the transient alteration of protein expression occurs during the first expansion, including, for example in the TIL population expanded in for example, Step B as indicated in FIG. 8 (for example FIG. 8A and/or FIG. 8B and/or FIG. 8C and/or FIG. 8D). In some embodiments, the transient alteration of protein expression occurs after the first expansion, including, for example in the TIL population in transition between the first and second expansion (e.g. the second population of TILs as described herein), the TIL population obtained from for example, Step B and included in Step C as indicated in FIG. 8. In some embodiments, the transient alteration of protein expression occurs in the bulk TIL population prior to second expansion, including, for example in the TIL population obtained from for example, Step C and prior to its expansion in Step D as indicated in FIG. 8. In some embodiments, the transient alteration of protein expression occurs during the second expansion, including, for example in the TIL population expanded in for example, Step D as indicated in FIG. 8 (e.g. the third population of TILs). In some embodiments, the transient alteration of protein expression occurs after the second expansion, including, for example in the TIL population obtained from the expansion in for example, Step D as indicated in FIG. 8.

In some embodiments, a method of transiently altering protein expression in a population of TILs includes the step of electroporation. Electroporation methods are known in the art and are described, e.g., in Tsong, Biophys. J 1991, 60, 297-306, and U.S. Patent Application Publication No. 2014/0227237 A1, the disclosures of each of which are incorporated by reference herein. In some embodiments, a method of transiently altering protein expression in population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of transiently altering protein expression in a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of transiently altering protein expression in a population of TILs includes the step of transfection using methods described in U.S. Pat. Nos. 5,766,902; 6,025,337; 6,410,517; 6,475,994; and 7,189,705; the disclosures of each of which are incorporated by reference herein.

In some embodiments, transient alteration of protein expression results in an increase in stem memory T cells (TSCMs). TSCMs are early progenitors of antigen-experienced central memory T cells. TSCMs generally display the long-term survival, self-renewal, and multipotency abilities that define stem cells, and are generally desirable for the generation of effective TIL products. TSCM have shown enhanced anti-tumor activity compared with other T cell subsets in mouse models of adoptive cell transfer. In some embodiments, transient alteration of protein expression results in a TIL population with a composition comprising a high proportion of TSCM. In some embodiments, transient alteration of protein expression results in an at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% increase in TSCM percentage. In some embodiments, transient alteration of protein expression results in an at least a 1-fold, 2-fold, 3-fold, 4-fold, 5-fold, or 10-fold increase in TSCMs in the TIL population. In some embodiments, transient alteration of protein expression results in a TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs. In some embodiments, transient alteration of protein expression results in a therapeutic TIL population with at least at least 5%, at least 10%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% TSCMs.

In some embodiments, transient alteration of protein expression results in rejuvenation of antigen-experienced T-cells. In some embodiments, rejuvenation includes, for example, increased proliferation, increased T-cell activation, and/or increased antigen recognition.

In some embodiments, transient alteration of protein expression alters the expression in a large fraction of the T-cells in order to preserve the tumor-derived TCR repertoire. In some embodiments, transient alteration of protein expression does not alter the tumor-derived TCR repertoire. In some embodiments, transient alteration of protein expression maintains the tumor-derived TCR repertoire.

In some embodiments, transient alteration of protein results in altered expression of a particular gene. In some embodiments, the transient alteration of protein expression targets a gene including but not limited to PD-1 (also referred to as PDCD1 or CC279), TGFBR2, CCR4/5, CBLB (CBL-B), CISH, CCRs (chimeric co-stimulatory receptors), IL-2, IL-12, IL-15, IL-21, NOTCH 1/2 ICD, CTLA-4, TIM3, LAG3, TIGIT, TET2, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, thymocyte selection associated high mobility group (HMG) box (TOX), ankyrin repeat domain 11 (ANKRD11), BCL6 co-repressor (BCOR) and/or cAMP protein kinase A (PKA). In some embodiments, the transient alteration of protein expression targets a gene selected from the group consisting of PD-1, TGFBR2, CCR4/5, CTLA-4, CBLB (CBL-B), CISH, CCRs (chimeric co-stimulatory receptors), IL-2, IL-12, IL-15, IL-21, NOTCH 1/2 ICD, TIM3, LAG3, TIGIT, TET2, TGFβ, CCR2, CCR4, CCR5, CXCR1, CXCR2, CSCR3, CCL2 (MCP-1), CCL3 (MIP-1α), CCL4 (MIP1-β), CCL5 (RANTES), CXCL1/CXCL8, CCL22, CCL17, CXCL1/CXCL8, VHL, CD44, PIK3CD, SOCS1, thymocyte selection associated high mobility group (HMG) box (TOX), ankyrin repeat domain 11 (ANKRD11), BCL6 co-repressor (BCOR) and/or cAMP protein kinase A (PKA). In some embodiments, the transient alteration of protein expression targets PD-1. In some embodiments, the transient alteration of protein expression targets TGFBR2. In some embodiments, the transient alteration of protein expression targets CCR4/5. In some embodiments, the transient alteration of protein expression targets CTLA-4. In some embodiments, the transient alteration of protein expression targets CBLB. In some embodiments, the transient alteration of protein expression targets CISH. In some embodiments, the transient alteration of protein expression targets CCRs (chimeric co-stimulatory receptors). In some embodiments, the transient alteration of protein expression targets IL-2. In some embodiments, the transient alteration of protein expression targets IL-12. In some embodiments, the transient alteration of protein expression targets IL-15. In some embodiments, the transient alteration of protein expression targets IL-21. In some embodiments, the transient alteration of protein expression targets NOTCH 1/2 ICD. In some embodiments, the transient alteration of protein expression targets TIM3. In some embodiments, the transient alteration of protein expression targets LAG3. In some embodiments, the transient alteration of protein expression targets TIGIT. In some embodiments, the transient alteration of protein expression targets TET2. In some embodiments, the transient alteration of protein expression targets TGFβ. In some embodiments, the transient alteration of protein expression targets CCR1. In some embodiments, the transient alteration of protein expression targets CCR2. In some embodiments, the transient alteration of protein expression targets CCR4. In some embodiments, the transient alteration of protein expression targets CCR5. In some embodiments, the transient alteration of protein expression targets CXCR1. In some embodiments, the transient alteration of protein expression targets CXCR2. In some embodiments, the transient alteration of protein expression targets CSCR3. In some embodiments, the transient alteration of protein expression targets CCL2 (MCP-1). In some embodiments, the transient alteration of protein expression targets CCL3 (MIP-1α). In some embodiments, the transient alteration of protein expression targets CCL4 (MIP1-β). In some embodiments, the transient alteration of protein expression targets CCL5 (RANTES). In some embodiments, the transient alteration of protein expression targets CXCL1. In some embodiments, the transient alteration of protein expression targets CXCL8. In some embodiments, the transient alteration of protein expression targets CCL22. In some embodiments, the transient alteration of protein expression targets CCL17. In some embodiments, the transient alteration of protein expression targets VHL. In some embodiments, the transient alteration of protein expression targets CD44. In some embodiments, the transient alteration of protein expression targets PIK3CD. In some embodiments, the transient alteration of protein expression targets SOCS1. In some embodiments, the transient alteration of protein expression targets thymocyte selection associated high mobility group (HMG) box (TOX). In some embodiments, the transient alteration of protein expression targets ankyrin repeat domain 11 (ANKRD11). In some embodiments, the transient alteration of protein expression targets BCL6 co-repressor (BCOR). In some embodiments, the transient alteration of protein expression targets cAMP protein kinase A (PKA).

In some embodiments, the transient alteration of protein expression results in increased and/or overexpression of a chemokine receptor. In some embodiments, the chemokine receptor that is overexpressed by transient protein expression includes a receptor with a ligand that includes but is not limited to CCL2 (MCP-1), CCL3 (MIP-1α), CCL4 (MIP1-β), CCL5 (RANTES), CXCL1, CXCL8, CCL22, and/or CCL17.

In some embodiments, the transient alteration of protein expression results in a decrease and/or reduced expression of PD-1, CTLA-4, CBLB, CISH, TIM-3, LAG-3, TIGIT, TET2, TGFβR2, and/or TGFβ (including resulting in, for example, TGFβ pathway blockade). In some embodiments, the transient alteration of protein expression results in a decrease and/or reduced expression of PD-1. In some embodiments, the transient alteration of protein expression results in a decrease and/or reduced expression of CBLB (CBL-B). In some embodiments, the transient alteration of protein expression results in a decrease and/or reduced expression of CISH. In some embodiments, the transient alteration of protein expression results in a decrease and/or reduced expression of TIM-3. In some embodiments, the transient alteration of protein expression results in a decrease and/or reduced expression of LAG-3. In some embodiments, the transient alteration of protein expression results in a decrease and/or reduced expression of TIGIT. In some embodiments, the transient alteration of protein expression results in a decrease and/or reduced expression of TET2. In some embodiments, the transient alteration of protein expression results in a decrease and/or reduced expression of TGFβR2. In some embodiments, the transient alteration of protein expression results in a decrease and/or reduced expression of TGFβ.

In some embodiments, the transient alteration of protein expression results in increased and/or overexpression of chemokine receptors in order to, for example, improve TIL trafficking or movement to the tumor site. In some embodiments, the transient alteration of protein expression results in increased and/or overexpression of a CCR (chimeric co-stimulatory receptor). In some embodiments, the transient alteration of protein expression results in increased and/or overexpression of a chemokine receptor selected from the group consisting of CCR1, CCR2, CCR4, CCR5, CXCR1, CXCR2, and/or CSCR3.

In some embodiments, the transient alteration of protein expression results in increased and/or overexpression of an interleukin. In some embodiments, the transient alteration of protein expression results in increased and/or overexpression of an interleukin selected from the group consisting of IL-2, IL-12, IL-15, IL-18 and/or IL-21.

In some embodiments, the transient alteration of protein expression results in increased and/or overexpression of NOTCH 1/2 ICD. In some embodiments, the transient alteration of protein expression results in increased and/or overexpression of VHL. In some embodiments, the transient alteration of protein expression results in increased and/or overexpression of CD44. In some embodiments, the transient alteration of protein expression results in increased and/or overexpression of PIK3CD. In some embodiments, the transient alteration of protein expression results in increased and/or overexpression of SOCS1.

In some embodiments, the transient alteration of protein expression results in decreased and/or reduced expression of cAMP protein kinase A (PKA).

In some embodiments, the transient alteration of protein expression results in decreased and/or reduced expression of a molecule selected from the group consisting of PD-1, LAG3, TIM3, CTLA-4, TIGIT, TET2, CISH, TGFβR2, PKA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the transient alteration of protein expression results in decreased and/or reduced expression of two molecules selected from the group consisting of PD-1, LAG3, TIM3, CTLA-4, TIGIT, TET2, CISH, TGFβR2, PKA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the transient alteration of protein expression results in decreased and/or reduced expression of PD-1 and one molecule selected from the group consisting of LAG3, TIM3, CTLA-4, TIGIT, TET2, CISH, TGFβR2, PKA, CBLB, BAFF (BR3), and combinations thereof (e.g., two or more of the above immune checkpoints, such as PD-1 and TIGIT, PD-1 and LAG3, PD-1 and TIM3, TIM3 and CTLA-4, etc.). In some embodiments, the transient alteration of protein expression results in decreased and/or reduced expression of PD-1, CTLA-4, LAG-3, CISH, CBLB, TIM3, TIGIT and combinations thereof (e.g., two or more of the above immune checkpoints, such as PD-1 and TIGIT, PD-1 and LAG3, PD-1 and TIM3, TIM3 and CTLA-4, etc.). In some embodiments, the transient alteration of protein expression results in decreased and/or reduced expression of PD-1 and one of CTLA-4, LAG3, CISH, CBLB, TIM3, TIGIT, TET2, and combinations thereof. In some embodiments, the transient alteration of protein expression results in decreased and/or reduced expression of PD-1 and CTLA-4. In some embodiments, the transient alteration of protein expression results in decreased and/or reduced expression of PD-1 and LAG3. In some embodiments, the transient alteration of protein expression results in decreased and/or reduced expression of PD-1 and CISH. In some embodiments, the transient alteration of protein expression results in decreased and/or reduced expression of PD-1 and CBLB. In some embodiments, the transient alteration of protein expression results in decreased and/or reduced expression of PD-1 and TIM3. In some embodiments, the transient alteration of protein expression results in decreased and/or reduced expression of PD-1 and TIGIT. In some embodiments, the transient alteration of protein expression results in decreased and/or reduced expression of PD-1 and TET2. In some embodiments, the transient alteration of protein expression results in decreased and/or reduced expression of CTLA-4 and LAG3. In some embodiments, the transient alteration of protein expression results in decreased and/or reduced expression of CTLA-4 and CISH. In some embodiments, the transient alteration of protein expression results in decreased and/or reduced expression of CTLA-4 and CBLB. In some embodiments, the transient alteration of protein expression results in decreased and/or reduced expression of CTLA-4 and TIM3. In some embodiments, the transient alteration of protein expression results in decreased and/or reduced expression of CTLA-4 and TIGIT. In some embodiments, the transient alteration of protein expression results in decreased and/or reduced expression of CTLA-4 and TET2. In some embodiments, the transient alteration of protein expression results in decreased and/or reduced expression of LAG3 and CISH. In some embodiments, the transient alteration of protein expression results in decreased and/or reduced expression of LAG3 and CBLB. In some embodiments, the transient alteration of protein expression results in decreased and/or reduced expression of LAG3 and TIM3. In some embodiments, the transient alteration of protein expression results in decreased and/or reduced expression of LAG3 and TIGIT. In some embodiments, the transient alteration of protein expression results in decreased and/or reduced expression of LAG3 and TET2. In some embodiments, the transient alteration of protein expression results in decreased and/or reduced expression of CISH and CBLB. In some embodiments, the transient alteration of protein expression results in decreased and/or reduced expression of CISH and TIM3. In some embodiments, the transient alteration of protein expression results in decreased and/or reduced expression of CISH and TIGIT. In some embodiments, the transient alteration of protein expression results in decreased and/or reduced expression of CISH and TET2. In some embodiments, the transient alteration of protein expression results in decreased and/or reduced expression of CBLB and TIM3. In some embodiments, the transient alteration of protein expression results in decreased and/or reduced expression of CBLB and TIGIT. In some embodiments, the transient alteration of protein expression results in decreased and/or reduced expression of CBLB and TET2. In some embodiments, the transient alteration of protein expression results in decreased and/or reduced expression of TIM3 and PD-1. In some embodiments, the transient alteration of protein expression results in decreased and/or reduced expression of TIM3 and LAG3. In some embodiments, the transient alteration of protein expression results in decreased and/or reduced expression of TIM3 and CISH. In some embodiments, the transient alteration of protein expression results in decreased and/or reduced expression of TIM3 and CBLB. In some embodiments, the transient alteration of protein expression results in decreased and/or reduced expression of TIM3 and TIGIT. In some embodiments, the transient alteration of protein expression results in decreased and/or reduced expression of TIM3 and TET2.

In some embodiments, an adhesion molecule selected from the group consisting of CCR2, CCR4, CCR5, CXCR2, CXCR3, CX3CR1, and combinations thereof, is inserted by a gammaretroviral or lentiviral method into the first population of TILs, second population of TILs, or harvested population of TILs (e.g., the expression of the adhesion molecule is increased).

In some embodiments, the transient alteration of protein expression results in decreased and/or reduced expression of a molecule selected from the group consisting of PD-1, LAG3, TIM3, CTLA-4, TIGIT, TET2, CISH, TGFβR2, PKA, CBLB, BAFF (BR3), and combinations thereof, and increased and/or enhanced expression of CCR2, CCR4, CCR5, CXCR2, CXCR3, CX3CR1, and combinations thereof. In some embodiments, the transient alteration of protein expression results in decreased and/or reduced expression of a molecule selected from the group consisting of PD-1, CTLA-4, LAG3, TIM3, CISH, CBLB, TIGIT, TET2 and combinations thereof, and increased and/or enhanced expression of CCR2, CCR4, CCR5, CXCR2, CXCR3, CX3CR1, and combinations thereof.

In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%, In some embodiments, there is a reduction in expression of at least about 90%. In some embodiments, there is a reduction in expression of at least about 95%. In some embodiments, there is a reduction in expression of at least about 99%.

In some embodiments, there is an increase in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is an increase in expression of at least about 65%, about 70%, about 75%, about 80%, about 85%, about 90%, or about 95%. In some embodiments, there is an increase in expression of at least about 75%, about 80%, about 85%, about 90%, or about 95%. In some embodiments, there is an increase in expression of at least about 80%, about 85%, about 90%, or about 95%. In some embodiments, there is an increase in expression of at least about 85%, about 90%, or about 95%. In some embodiments, there is an increase in expression of at least about 80%. In some embodiments, there is an increase in expression of at least about 85%, In some embodiments, there is an increase in expression of at least about 90%. In some embodiments, there is an increase in expression of at least about 95%. In some embodiments, there is an increase in expression of at least about 99%.

In some embodiments, transient alteration of protein expression is induced by treatment of the TILs with transcription factors (TFs) and/or other molecules capable of transiently altering protein expression in the TILs. In some embodiments, the SQZ vector-free microfluidic platform is employed for intracellular delivery of the transcription factors (TFs) and/or other molecules capable of transiently altering protein expression. Such methods demonstrating the ability to deliver proteins, including transcription factors, to a variety of primary human cells, including T cells, which have been described in U.S. Patent Application Publication Nos. US 2019/0093073 A1, US 2018/0201889 A1, and US 2019/0017072 A1, the disclosures of each of which are incorporated herein by reference. Such methods can be employed with the present invention in order to expose a population of TILs to transcription factors (TFs) and/or other molecules capable of inducing transient protein expression, wherein said TFs and/or other molecules capable of inducing transient protein expression provide for increased expression of tumor antigens and/or an increase in the number of tumor antigen-specific T cells in the population of TILs, thus resulting in reprogramming of the TIL population and an increase in therapeutic efficacy of the reprogrammed TIL population as compared to a non-reprogrammed TIL population. In some embodiments, the reprogramming results in an increased subpopulation of effector T cells and/or central memory T cells relative to the starting or prior population (i.e., prior to reprogramming) population of TILs, as described herein.

In some embodiments, the transcription factor (TF) includes but is not limited to TCF-1, NOTCH 1/2 ICD, and/or MYB. In some embodiments, the transcription factor (TF) is TCF-1. In some embodiments, the transcription factor (TF) is NOTCH 1/2 ICD. In some embodiments, the transcription factor (TF) is MYB. In some embodiments, the transcription factor (TF) is administered with induced pluripotent stem cell culture (iPSC), such as the commercially available KNOCKOUT Serum Replacement (Gibco/ThermoFisher), to induce additional TIL reprogramming. In some embodiments, the transcription factor (TF) is administered with an iPSC cocktail to induce additional TIL reprogramming. In some embodiments, the transcription factor (TF) is administered without an iPSC cocktail. In some embodiments, reprogramming results in an increase in the percentage of TSCMs. In some embodiments, reprogramming results in an increase in the percentage of TSCMs by about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95% TSCMs.

In some embodiments, a method of transient altering protein expression, as described above, may be combined with a method of genetically modifying a population of TILs includes the step of stable incorporation of genes for production of one or more proteins. In certain embodiments, the method comprises a step of genetically modifying a population of TILs. In certain embodiments, the method comprises genetically modifying the first population of TILs, the second population of TILs and/or the third population of TILs. In some embodiments, a method of genetically modifying a population of TILs includes the step of retroviral transduction. In some embodiments, a method of genetically modifying a population of TILs includes the step of lentiviral transduction. Lentiviral transduction systems are known in the art and are described, e.g., in Levine, et al., Proc. Nat'l Acad. Sci. 2006, 103, 17372-77; Zufferey, et al., Nat. Biotechnol. 1997, 15, 871-75; Dull, et al., J. Virology 1998, 72, 8463-71, and U.S. Pat. No. 6,627,442, the disclosures of each of which are incorporated by reference herein. In some embodiments, a method of genetically modifying a population of TILs includes the step of gamma-retroviral transduction. Gamma-retroviral transduction systems are known in the art and are described, e.g., Cepko and Pear, Cur. Prot. Mol. Biol. 1996, 9.9.1-9.9.16, the disclosure of which is incorporated by reference herein. In some embodiments, a method of genetically modifying a population of TILs includes the step of transposon-mediated gene transfer. Transposon-mediated gene transfer systems are known in the art and include systems wherein the transposase is provided as DNA expression vector or as an expressible RNA or a protein such that long-term expression of the transposase does not occur in the transgenic cells, for example, a transposase provided as an mRNA (e.g., an mRNA comprising a cap and poly-A tail). Suitable transposon-mediated gene transfer systems, including the salmonid-type Tel-like transposase (SB or Sleeping Beauty transposase), such as SB10, SB11, and SB100x, and engineered enzymes with increased enzymatic activity, are described in, e.g., Hackett, et al., Mol. Therapy 2010, 18, 674-83 and U.S. Pat. No. 6,489,458, the disclosures of each of which are incorporated by reference herein.

In some embodiments, transient alteration of protein expression in TILs is induced by small interfering RNA (siRNA), sometimes known as short interfering RNA or silencing RNA, which is a double stranded RNA molecule, generally 19-25 base pairs in length. siRNA is used in RNA interference (RNAi), where it interferes with expression of specific genes with complementary nucleotide sequences.

In some embodiments, transient alteration of protein expression is a reduction in expression. In some embodiments, transient alteration of protein expression in TILs is induced by self-delivering RNA interference (sdRNA), which is a chemically-synthesized asymmetric siRNA duplex with a high percentage of 2′-OH substitutions (typically fluorine or —OCH₃) which comprises a 20-nucleotide antisense (guide) strand and a 13 to 15 base sense (passenger) strand conjugated to cholesterol at its 3′ end using a tetraethylenglycol (TEG) linker. Small interfering RNA (siRNA), sometimes known as short interfering RNA or silencing RNA, is a double stranded RNA molecule, generally 19-25 base pairs in length. siRNA is used in RNA interference (RNAi), where it interferes with expression of specific genes with complementary nucleotide sequences. sdRNA are covalently and hydrophobically modified RNAi compounds that do not require a delivery vehicle to enter cells. sdRNAs are generally asymmetric chemically modified nucleic acid molecules with minimal double stranded regions. sdRNA molecules typically contain single stranded regions and double stranded regions and can contain a variety of chemical modifications within both the single stranded and double stranded regions of the molecule. Additionally, the sdRNA molecules can be attached to a hydrophobic conjugate such as a conventional and advanced sterol-type molecule, as described herein. sdRNAs and associated methods for making such sdRNAs have also been described extensively in, for example, U.S. Patent Application Publication Nos. US 2016/0304873 A1, US 2019/0211337 A1, US 2009/0131360 A1, and US 2019/0048341 A1, and U.S. Pat. No. 10,633,654 and 10,913,948B2, the disclosures of each of which are incorporated by reference herein. To optimize sdRNA structure, chemistry, targeting position, sequence preferences, and the like, an algorithm has been developed and utilized for sdRNA potency prediction. Based on these analyses, functional sdRNA sequences have been generally defined as having over 70% reduction in expression at 1 μM concentration, with a probability over 40%.

Double stranded RNA (dsRNA) can be generally used to define any molecule comprising a pair of complementary strands of RNA, generally a sense (passenger) and antisense (guide) strands, and may include single-stranded overhang regions. The term dsRNA, contrasted with siRNA, generally refers to a precursor molecule that includes the sequence of an siRNA molecule which is released from the larger dsRNA molecule by the action of cleavage enzyme systems, including Dicer.

In some embodiments, the method comprises transient alteration of protein expression in a population of TILs, including TILs modified to express a CCR, comprising the use of self-delivering RNA interference (sdRNA), which is for example, a chemically-synthesized asymmetric siRNA duplex with a high percentage of 2′-OH substitutions (typically fluorine or —OCH₃) which comprises a 20-nucleotide antisense (guide) strand and a 13 to 15 base sense (passenger) strand conjugated to cholesterol at its 3′ end using a tetraethylenglycol (TEG) linker. Methods of using siRNA and sdRNA have been described in Khvorova and Watts, Nat. Biotechnol. 2017, 35, 238-248; Byrne, et al., J Ocul. Pharmacol. Ther. 2013, 29, 855-864; and Ligtenberg, et al., Mol. Therapy, 2018, 26, 1482-93, the disclosures of which are incorporated by reference herein. In some embodiments, delivery of siRNA is accomplished using electroporation or cell membrane disruption (such as the squeeze or SQZ method). In some embodiments, delivery of sdRNA to a TIL population is accomplished without use of electroporation, SQZ, or other methods, instead using a 1 to 3 day period in which a TIL population is exposed to sdRNA at a concentration of 1 μM/10,000 TILs in medium. In certain embodiments, the method comprises delivery or siRNA or sdRNA to a TILs population comprising exposing the TILs population to sdRNA at a concentration of 1 μM/10,000 TILs in medium for a period of between 1 to 3 days. In some embodiments, delivery of sdRNA to a TIL population is accomplished using a 1 to 3 day period in which a TIL population is exposed to sdRNA at a concentration of 10 μM/10,000 TILs in medium. In some embodiments, delivery of sdRNA to a TIL population is accomplished using a 1 to 3 day period in which a TIL population is exposed to sdRNA at a concentration of 50 μM/10,000 TILs in medium. In some embodiments, delivery of sdRNA to a TIL population is accomplished using a 1 to 3 day period in which a TIL population is exposed to sdRNA at a concentration of between 0.1 μM/10,000 TILs and 50 μM/10,000 TILs in medium. In some embodiments, delivery of sdRNA to a TIL population is accomplished using a 1 to 3 day period in which a TIL population is exposed to sdRNA at a concentration of between 0.1 μM/10,000 TILs and 50 μM/10,000 TILs in medium, wherein the exposure to sdRNA is performed two, three, four, or five times by addition of fresh sdRNA to the media. Other suitable processes are described, for example, in U.S. Patent Application Publication No. US 2011/0039914 A1, US 2013/0131141 A1, and US 2013/0131142 A1, and U.S. Pat. No. 9,080,171, the disclosures of which are incorporated by reference herein.

In some embodiments, siRNA or sdRNA is inserted into a population of TILs during manufacturing. In some embodiments, the sdRNA encodes RNA that interferes with NOTCH 1/2 ICD, PD-1, CTLA-4 TIM-3, LAG-3, TIGIT, TGFβ, TGFBR2, cAMP protein kinase A (PKA), BAFF BR3, CISH, and/or CBLB. In some embodiments, the reduction in expression is determined based on a percentage of gene silencing, for example, as assessed by flow cytometry and/or qPCR. In some embodiments, there is a reduction in expression of about 5%, about 10%, about 10%, about 20%, about 25%, about 30%, about 35%, about 40%, about 45%, about 50%, about 55%, about 60%, about 65%, about 70%, about 75%, about 80%, about 85%, about 90%, or about 95%. In some embodiments, there is a reduction in expression of at least about 65%, about 70%, about 75%, about 80%, about 85%, about 90%, or about 95%. In some embodiments, there is a reduction in expression of at least about 75%, about 80%, about 85%, about 90%, or about 95%. In some embodiments, there is a reduction in expression of at least about 80%, about 85%, about 90%, or about 95%. In some embodiments, there is a reduction in expression of at least about 85%, about 90%, or about 95%. In some embodiments, there is a reduction in expression of at least about 80%. In some embodiments, there is a reduction in expression of at least about 85%, In some embodiments, there is a reduction in expression of at least about 90%. In some embodiments, there is a reduction in expression of at least about 95%. In some embodiments, there is a reduction in expression of at least about 99%.

The self-deliverable RNAi technology based on the chemical modification of siRNAs can be employed with the methods of the present invention to successfully deliver the sdRNAs to the TILs as described herein. The combination of backbone modifications with asymmetric siRNA structure and a hydrophobic ligand (see, for example, Ligtenberg, et al., Mol. Therapy, 2018, 26, 1482-93 and U.S. Patent Application Publication No. 2016/0304873 A1, the disclosures of which are incorporated by reference herein) allow sdRNAs to penetrate cultured mammalian cells without additional formulations and methods by simple addition to the culture media, capitalizing on the nuclease stability of sdRNAs. This stability allows the support of constant levels of RNAi-mediated reduction of target gene activity simply by maintaining the active concentration of sdRNA in the media. While not being bound by theory, the backbone stabilization of sdRNA provides for extended reduction in gene expression effects which can last for months in non-dividing cells.

In some embodiments, over 95% transfection efficiency of TILs and a reduction in expression of the target by various specific siRNAs or sdRNAs occurs. In some embodiments, siRNAs or sdRNAs containing several unmodified ribose residues were replaced with fully modified sequences to increase potency and/or the longevity of RNAi effect. In some embodiments, a reduction in expression effect is maintained for 12 hours, 24 hours, 36 hours, 48 hours, 5 days, 6 days, 7 days, or 8 days or more. In some embodiments, the reduction in expression effect decreases at 10 days or more post siRNA or sdRNA treatment of the TILs. In some embodiments, more than 70% reduction in expression of the target expression is maintained. In some embodiments, more than 70% reduction in expression of the target expression is maintained TILs. In some embodiments, a reduction in expression in the PD-1/PD-L1 pathway allows for the TILs to exhibit a more potent in vivo effect, which is in some embodiments, due to the avoidance of the suppressive effects of the PD-1/PD-L1 pathway. In some embodiments, a reduction in expression of PD-1 by siRNA or sdRNA results in an increase TIL proliferation.

In some embodiments, the sdRNA sequences used in the invention exhibit a 70% reduction in expression of the target gene. In some embodiments, the sdRNA sequences used in the invention exhibit a 75% reduction in expression of the target gene. In some embodiments, the sdRNA sequences used in the invention exhibit an 80% reduction in expression of the target gene. In some embodiments, the sdRNA sequences used in the invention exhibit an 85% reduction in expression of the target gene. In some embodiments, the sdRNA sequences used in the invention exhibit a 90% reduction in expression of the target gene. In some embodiments, the sdRNA sequences used in the invention exhibit a 95% reduction in expression of the target gene. In some embodiments, the sdRNA sequences used in the invention exhibit a 99% reduction in expression of the target gene. In some embodiments, the sdRNA sequences used in the invention exhibit a reduction in expression of the target gene when delivered at a concentration of about 0.25 μM to about 4 μM. In some embodiments, the sdRNA sequences used in the invention exhibit a reduction in expression of the target gene when delivered at a concentration of about 0.25 μM. In some embodiments, the sdRNA sequences used in the invention exhibit a reduction in expression of the target gene when delivered at a concentration of about 0.5 μM. In some embodiments, the sdRNA sequences used in the invention exhibit a reduction in expression of the target gene when delivered at a concentration of about 0.75 μM. In some embodiments, the sdRNA sequences used in the invention exhibit a reduction in expression of the target gene when delivered at a concentration of about 1.0 μM. In some embodiments, the sdRNA sequences used in the invention exhibit a reduction in expression of the target gene when delivered at a concentration of about 1.25 μM. In some embodiments, the sdRNA sequences used in the invention exhibit a reduction in expression of the target gene when delivered at a concentration of about 1.5 μM. In some embodiments, the sdRNA sequences used in the invention exhibit a reduction in expression of the target gene when delivered at a concentration of about 1.75 μM. In some embodiments, the sdRNA sequences used in the invention exhibit a reduction in expression of the target gene when delivered at a concentration of about 2.0 μM. In some embodiments, the sdRNA sequences used in the invention exhibit a reduction in expression of the target gene when delivered at a concentration of about 2.25 μM. In some embodiments, the sdRNA sequences used in the invention exhibit a reduction in expression of the target gene when delivered at a concentration of about 2.5 μM. In some embodiments, the sdRNA sequences used in the invention exhibit a reduction in expression of the target gene when delivered at a concentration of about 2.75 μM. In some embodiments, the sdRNA sequences used in the invention exhibit a reduction in expression of the target gene when delivered at a concentration of about 3.0 μM. In some embodiments, the sdRNA sequences used in the invention exhibit a reduction in expression of the target gene when delivered at a concentration of about 3.25 μM. In some embodiments, the sdRNA sequences used in the invention exhibit a reduction in expression of the target gene when delivered at a concentration of about 3.5 μM. In some embodiments, the sdRNA sequences used in the invention exhibit a reduction in expression of the target gene when delivered at a concentration of about 3.75 μM. In some embodiments, the sdRNA sequences used in the invention exhibit a reduction in expression of the target gene when delivered at a concentration of about 4.0 μM.

In some embodiments, the siRNA or sdRNA oligonucleotide agents comprise one or more modification to increase stability and/or effectiveness of the therapeutic agent, and to effect efficient delivery of the oligonucleotide to the cells or tissue to be treated. Such modifications can include a 2′-O-methyl modification, a 2′-O-fluro modification, a diphosphorothioate modification, 2′ F modified nucleotide, a2′—O-methyl modified and/or a 2′deoxy nucleotide. In some embodiments, the oligonucleotide is modified to include one or more hydrophobic modifications including, for example, sterol, cholesterol, vitamin D, naphtyl, isobutyl, benzyl, indol, tryptophane, and/or phenyl. In some embodiments, chemically modified nucleotides are combination of phosphorothioates, 2′-O-methyl, 2′deoxy, hydrophobic modifications and phosphorothioates. In some embodiments, the sugars can be modified and modified sugars can include but are not limited to D-ribose, 2′-O-alkyl (including 2′-O-methyl and 2′-O-ethyl), i.e., 2′-alkoxy, 2-amino, 2′-S-alkyl, 2′-halo (including 2′-fluoro), T-methoxyethoxy, 2′-allyloxy (—OCH₂CH═CH₂), 2′-propargyl, 2′-propyl, ethynyl, ethenyl, propenyl, and cyano and the like. In some embodiments, the sugar moiety can be a hexose and incorporated into an oligonucleotide as described in Augustyns, et al., Nucl. Acids. Res. 1992, 18, 4711, the disclosure of which is incorporated by reference herein.

In some embodiments, the double-stranded siRNA or sdRNA oligonucleotide of the invention is double-stranded over its entire length, i.e., with no overhanging single-stranded sequence at either end of the molecule, i.e., is blunt-ended. In some embodiments, the individual nucleic acid molecules can be of different lengths. In other words, a double-stranded siRNA or sdRNA oligonucleotide of the invention is not double-stranded over its entire length. For instance, when two separate nucleic acid molecules are used, one of the molecules, e.g., the first molecule comprising an antisense sequence, can be longer than the second molecule hybridizing thereto (leaving a portion of the molecule single-stranded). In some embodiments, when a single nucleic acid molecule is used a portion of the molecule at either end can remain single-stranded.

In some embodiments, a double-stranded siRNA or sdRNA oligonucleotide of the invention contains mismatches and/or loops or bulges, but is double-stranded over at least about 70% of the length of the oligonucleotide. In some embodiments, a double-stranded oligonucleotide of the invention is double-stranded over at least about 80% of the length of the oligonucleotide. In other embodiments, a double-stranded siRNA or sdRNA oligonucleotide of the invention is double-stranded over at least about 90%-95% of the length of the oligonucleotide. In some embodiments, a double-stranded siRNA or sdRNA oligonucleotide of the invention is double-stranded over at least about 96%-98% of the length of the oligonucleotide. In some embodiments, the double-stranded oligonucleotide of the invention contains at least or up to 1, 2, 3, 4, 5, 6, 7, 8, 9, 10, 11, 12, 13, 14, or 15 mismatches.

In some embodiments, the siRNA or sdRNA oligonucleotide can be substantially protected from nucleases e.g., by modifying the 3′ or 5′ linkages, as described in U.S. Pat. No. 5,849,902 and WO 98/13526, the disclosure of which are incorporated by reference herein. For example, oligonucleotides can be made resistant by the inclusion of a “blocking group.” The term “blocking group” as used herein refers to substituents (e.g., other than OH groups) that can be attached to oligonucleotides or nucleomonomers, either as protecting groups or coupling groups for synthesis (e.g., FITC, propyl (CH₂—CH₂—CH₃), glycol (—O—CH₂—CH₂—O—) phosphate (PO₃²″), hydrogen phosphonate, or phosphoramidite). “Blocking groups” can also include “end blocking groups” or “exonuclease blocking groups” which protect the 5′ and 3′ termini of the oligonucleotide, including modified nucleotides and non-nucleotide exonuclease resistant structures.

In some embodiments, at least a portion of the contiguous polynucleotides within the siRNA or sdRNA are linked by a substitute linkage, e.g., a phosphorothioate linkage.

In some embodiments, chemical modification can lead to at least a 1.5, 2, 3, 4, 5, 6, 7, 8, 9, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195, 200, 225, 250, 275, 300, 325, 350, 375, 400, 425, 450, 475, or 500 percent enhancement in cellular uptake of an siRNA or sdRNA. In some embodiments, at least one of the C or U residues includes a hydrophobic modification. In some embodiments, a plurality of Cs and Us contain a hydrophobic modification. In some embodiments, at least 10%, 15%, 20%, 30%, 40%, 50%, 55%, 60% 65%, 70%, 75%, 80%, 85%, 90% or at least 95% of the Cs and Us can contain a hydrophobic modification. In some embodiments, all of the Cs and Us contain a hydrophobic modification.

In some embodiments, the siRNA or sdRNA molecules exhibit enhanced endosomal release of through the incorporation of protonatable amines. In some embodiments, protonatable amines are incorporated in the sense strand (in the part of the molecule which is discarded after RISC loading). In some embodiments, the siRNA or sdRNA compounds of the invention comprise an asymmetric compound comprising a duplex region (required for efficient RISC entry of 10-15 bases long) and single stranded region of 4-12 nucleotides long; with a 13 nucleotide duplex. In some embodiments, a 6 nucleotide single stranded region is employed. In some embodiments, the single stranded region of the siRNA or sdRNA comprises 2-12 phosphorothioate intemucleotide linkages (referred to as phosphorothioate modifications). In some embodiments, 6-8 phosphorothioate internucleotide linkages are employed. In some embodiments, the siRNA or sdRNA compounds of the invention also include a unique chemical modification pattern, which provides stability and is compatible with RISC entry. The guide strand, for example, may also be modified by any chemical modification which confirms stability without interfering with RISC entry. In some embodiments, the chemical modification pattern in the guide strand includes the majority of C and U nucleotides being 2′ F modified and the 5′end being phosphorylated.

In some embodiments, at least 30% of the nucleotides in the siRNA or sdRNA are modified. In some embodiments, at least 30%, 31%, 32%, 33%, 34%, 35%, 36%, 37%, 38%, 39%, 40%4, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%7, 73%, 74%, 75%, 76%7, 77%, 78%, 79%, 80%, 81%, 82%, 83%, 84%, 85%, 86%, 87%, 88%, 89%, 90%, 91%, 92%, 93%, 94%, 95%, 96%, 97%, 98% or 99% of the nucleotides in the siRNA or sdRNA are modified. In some embodiments, 100% of the nucleotides in the siRNA or sdRNA are modified.

In some embodiments, the siRNA or sdRNA molecules have minimal double stranded regions. In some embodiments the region of the molecule that is double stranded ranges from 8-15 nucleotides long. In some embodiments, the region of the molecule that is double stranded is 8, 9, 10, 11, 12, 13, 14 or 15 nucleotides long. In some embodiments the double stranded region is 13 nucleotides long. There can be 100% complementarity between the guide and passenger strands, or there may be one or more mismatches between the guide and passenger strands. In some embodiments, on one end of the double stranded molecule, the molecule is either blunt-ended or has a one-nucleotide overhang. The single stranded region of the molecule is in some embodiments between 4-12 nucleotides long. In some embodiments, the single stranded region can be 4, 5, 6, 7, 8, 9, 10, 11 or 12 nucleotides long. In some embodiments, the single stranded region can also be less than 4 or greater than 12 nucleotides long. In certain embodiments, the single stranded region is 6 or 7 nucleotides long.

In some embodiments, the siRNA or sdRNA molecules have increased stability. In some instances, a chemically modified siRNA or sdRNA molecule has a half-life in media that is longer than 1, 2, 3, 4, 5, 6, 7, 8, 9, 10, 11, 12, 13, 14, 15, 16, 17, 18, 19, 20, 21, 22, 23, 24 or more than 24 hours, including any intermediate values. In some embodiments, the siRNA or sd-RNA has a half-life in media that is longer than 12 hours.

In some embodiments, the siRNA or sdRNA is optimized for increased potency and/or reduced toxicity. In some embodiments, nucleotide length of the guide and/or passenger strand, and/or the number of phosphorothioate modifications in the guide and/or passenger strand, can in some aspects influence potency of the RNA molecule, while replacing 2′-fluoro (2′F) modifications with 2′-O-methyl (2′OMe) modifications can in some aspects influence toxicity of the molecule. In some embodiments, reduction in 2′F content of a molecule is predicted to reduce toxicity of the molecule. In some embodiments, the number of phosphorothioate modifications in an RNA molecule can influence the uptake of the molecule into a cell, for example the efficiency of passive uptake of the molecule into a cell. In some embodiments, the siRNA or sdRNA has no 2′F modification and yet are characterized by equal efficacy in cellular uptake and tissue penetration.

In some embodiments, a guide strand is approximately 18-19 nucleotides in length and has approximately 2-14 phosphate modifications. For example, a guide strand can contain 2, 3, 4, 5, 6, 7, 8, 9, 10, 11, 12, 13, 14 or more than 14 nucleotides that are phosphate-modified. The guide strand may contain one or more modifications that confer increased stability without interfering with RISC entry. The phosphate modified nucleotides, such as phosphorothioate modified nucleotides, can be at the 3′ end, 5′ end or spread throughout the guide strand. In some embodiments, the 3′ terminal 10 nucleotides of the guide strand contain 1, 2, 3, 4, 5, 6, 7, 8, 9 or 10 phosphorothioate modified nucleotides. The guide strand can also contain 2′F and/or 2′OMe modifications, which can be located throughout the molecule. In some embodiments, the nucleotide in position one of the guide strand (the nucleotide in the most 5′ position of the guide strand) is 2′OMe modified and/or phosphorylated. C and U nucleotides within the guide strand can be 2′F modified. For example, C and U nucleotides in positions 2-10 of a 19 nt guide strand (or corresponding positions in a guide strand of a different length) can be 2′F modified. C and U nucleotides within the guide strand can also be 2′OMe modified. For example, C and U nucleotides in positions 11-18 of a 19 nt guide strand (or corresponding positions in a guide strand of a different length) can be 2′OMe modified. In some embodiments, the nucleotide at the most 3′ end of the guide strand is unmodified. In certain embodiments, the majority of Cs and Us within the guide strand are 2′F modified and the 5′ end of the guide strand is phosphorylated. In other embodiments, position 1 and the Cs or Us in positions 11-18 are 2′OMe modified and the 5′ end of the guide strand is phosphorylated. In other embodiments, position 1 and the Cs or Us in positions 11-18 are 2′OMe modified, the 5′ end of the guide strand is phosphorylated, and the Cs or Us in position 2-10 are 2′F modified.

The self-deliverable RNAi technology provides a method of directly transfecting cells with the RNAi agent (whether siRNA, sdRNA, or other RNAi agents), without the need for additional formulations or techniques. The ability to transfect hard-to-transfect cell lines, high in vivo activity, and simplicity of use, are characteristics of the compositions and methods that present significant functional advantages over traditional siRNA-based techniques, and as such, the sdRNA methods are employed in several embodiments related to the methods of reduction in expression of the target gene in the TILs of the present invention. The sdRNA method allows direct delivery of chemically synthesized compounds to a wide range of primary cells and tissues, both ex-vivo and in vivo. The sdRNAs described in some embodiments of the invention herein are commercially available from Advima LLC, Worcester, MA, USA.

siRNA and sdRNA may be formed as hydrophobically-modified siRNA-antisense oligonucleotide hybrid structures, and are disclosed, for example in Byrne, et al., J. Ocular Pharmacol. Therapeut., 2013, 29, 855-864, the disclosure of which is incorporated by reference herein.

In some embodiments, the siRNA or sdRNA oligonucleotides can be delivered to the TILs described herein using sterile electroporation. In certain embodiments, the method comprises sterile electroporation of a population of TILs to deliver siRNA or sdRNA oligonucleotides.

In some embodiments, the oligonucleotides can be delivered to the cells in combination with a transmembrane delivery system. In some embodiments, this transmembrane delivery system comprises lipids, viral vectors, and the like. In some embodiments, the oligonucleotide agent is a self-delivery RNAi agent, that does not require any delivery agents. In certain embodiments, the method comprises use of a transmembrane delivery system to deliver siRNA or sdRNA oligonucleotides to a population of TILs.

Oligonucleotides and oligonucleotide compositions are contacted with (e.g., brought into contact with, also referred to herein as administered or delivered to) and taken up by TILs described herein, including through passive uptake by TILs. The sdRNA can be added to the TILs as described herein during the first expansion, for example Step B, after the first expansion, for example, during Step C, before or during the second expansion, for example before or during Step D, after Step D and before harvest in Step E, during or after harvest in Step F, before or during final formulation and/or transfer to infusion Bag in Step F, as well as before any optional cryopreservation step in Step F. Moreover, sdRNA can be added after thawing from any cryopreservation step in Step F. In some embodiments, one or more sdRNAs targeting genes as described herein, including PD-1, LAG-3, TIM-3, CISH, CTLA-4, TIGIT, TET2 and CBLB, may be added to cell culture media comprising TILs and other agents at concentrations selected from the group consisting of 100 nM to 20 mM, 200 nM to 10 mM, 500 nm to 1 mM, 1 μM to 100 μM, and 1 μM to 100 μM. In some embodiments, one or more sdRNAs targeting genes as described herein, including PD-1, LAG-3, TIM-3, CISH, CTLA-4, TIGIT, TET2 and CBLB, may be added to cell culture media comprising TILs and other agents at amounts selected from the group consisting of 0.1 μM sdRNA/10,000 TILs/100 μL media, 0.5 μM sdRNA/10,000 TILs/100 μL media, 0.75 μM sdRNA/10,000 TILs/100 μL media, 1 μM sdRNA/10,000 TILs/100 μL media, 1.25 μM sdRNA/10,000 TILs/100 μL media, 1.5 μM sdRNA/10,000 TILs/100 μL media, 2 μM sdRNA/10,000 TILs/100 μL media, 5 μM sdRNA/10,000 TILs/100 μL media, or 10 μM sdRNA/10,000 TILs/100 μL media. In some embodiments, one or more sdRNAs targeting genes as described herein, including PD-1, LAG-3, TIM-3, CISH, CTLA-4, TIGIT, TET2 and CBLB, may be added to TIL cultures during the pre-REP or REP stages twice a day, once a day, every two days, every three days, every four days, every five days, every six days, or every seven days.

Oligonucleotide compositions of the invention, including sdRNA, can be contacted with TILs as described herein during the expansion process, for example by dissolving sdRNA at high concentrations in cell culture media and allowing sufficient time for passive uptake to occur. In certain embodiments, the method of the present invention comprises contacting a population of TILs with an oligonucleotide composition as described herein. In certain embodiments, the method comprises dissolving an oligonucleotide e.g., sdRNA in a cell culture media and contacting the cell culture media with a population of TILs. The TILs may be a first population, a second population and/or a third population as described herein.

In some embodiments, delivery of oligonucleotides into cells can be enhanced by suitable art recognized methods including calcium phosphate, DMSO, glycerol or dextran, electroporation, or by transfection, e.g., using cationic, anionic, or neutral lipid compositions or liposomes using methods known in the art, such as those methods described in U.S. Pat. Nos. 4,897,355; 5,459,127; 5,631,237; 5,955,365; 5,976,567; 10,087,464; and 10,155,945; and Bergan, et al., Nucl. Acids Res. 1993, 21, 3567, the disclosures of each of which are incorporated by reference herein.

In some embodiments, more than one siRNA or sdRNA is used to reduce expression of a target gene. In some embodiments, one or more of PD-1, TIM-3, CBLB, LAG3, CTLA-4, TIGIT, TET2 and/or CISH targeting siRNA or sdRNAs are used together. In some embodiments, a PD-1 siRNA or sdRNA is used with one or more of TIM-3, CBLB, LAG3, CTLA-4, TIGIT, TET2 and/or CISH in order to reduce expression of more than one gene target. In some embodiments, a LAG3 siRNA or sdRNA is used in combination with a CISH targeting siRNA or sdRNA to reduce gene expression of both targets. In some embodiments, the siRNAs or sdRNAs targeting one or more of PD-1, TIM-3, CBLB, LAG3, CTLA-4, TIGIT, TET2 and/or CISH herein are commercially available from Advima LLC, Worcester, MA, USA or multiple other vendors.

In some embodiments, the siRNA or sdRNA targets a gene selected from the group consisting of PD-1, LAG3, TIM3, CTLA-4, TIGIT, TET2, CISH, TGFβR2, PKA, CBLB, BAFF (BR3), and combinations thereof. In some embodiments, the siRNA or sdRNA targets a gene selected from the group consisting of PD-1, LAG3, TIM3, CTLA-4, TIGIT, TET2, CISH, TGFβR2, PKA, CBLB, BAFF (BR3), and combinations thereof. In some embodiments, one siRNA or sdRNA targets PD-1 and another siRNA or sdRNA targets a gene selected from the group consisting of LAG3, TIM3, CTLA-4, TIGIT, TET2, CISH, TGFβR2, PKA, CBLB, BAFF (BR3), and combinations thereof. In some embodiments, the siRNA or sdRNA targets a gene selected from PD-1, LAG-3, CISH, CBLB, TIM3, CTLA-4, TIGIT, TET2 and combinations thereof. In some embodiments, the siRNA or sdRNA targets a gene selected from PD-1 and one of LAG3, CISH, CBLB, TIM3, and combinations thereof. In some embodiments, one siRNA or sdRNA targets PD-1 and one siRNA or sdRNA targets LAG3. In some embodiments, one siRNA or sdRNA targets PD-1 and one siRNA or sdRNA targets CISH. In some embodiments, one siRNA or sdRNA targets PD-1 and one siRNA or sdRNA targets CBLB. In some embodiments, one siRNA or sdRNA targets PD-1 and one siRNA or sdRNA targets TIM3. In some embodiments, one siRNA or sdRNA targets PD-1 and one siRNA or sdRNA targets CTLA-4. In some embodiments, one siRNA or sdRNA targets PD-1 and one siRNA or sdRNA targets TIGIT. In some embodiments, one siRNA or sdRNA targets PD-1 and one siRNA or sdRNA targets TET2. In some embodiments, one siRNA or sdRNA targets LAG3 and one siRNA or sdRNA targets CISH. In some embodiments, one siRNA or sdRNA targets LAG3 and one siRNA or sdRNA targets CBLB. In some embodiments, one siRNA or sdRNA targets LAG3 and one siRNA or sdRNA targets TIM3. In some embodiments, one siRNA or sdRNA targets LAG3 and one siRNA or sdRNA targets CTLA-4. In some embodiments, one siRNA or sdRNA targets LAG3 and one siRNA or sdRNA targets TIGIT. In some embodiments, one siRNA or sdRNA targets LAG3 and one siRNA or sdRNA targets TET2. In some embodiments, one siRNA or sdRNA targets CISH and one siRNA or sdRNA targets CBLB. In some embodiments, one siRNA or sdRNA targets CISH and one siRNA or sdRNA targets TIM3. In some embodiments, one siRNA or sdRNA targets CISH and one siRNA or sdRNA targets CTLA-4. In some embodiments, one siRNA or sdRNA targets CISH and one siRNA or sdRNA targets TIGIT. In some embodiments, one siRNA or sdRNA targets CISH and one siRNA or sdRNA targets TET2. In some embodiments, one siRNA or sdRNA targets CBLB and one siRNA or sdRNA targets TIM3. In some embodiments, one siRNA or sdRNA targets CBLB and one siRNA or sdRNA targets CTLA-4. In some embodiments, one siRNA or sdRNA targets CBLB and one siRNA or sdRNA targets TIGIT. In some embodiments, one siRNA or sdRNA targets CBLB and one siRNA or sdRNA targets TET2. In some embodiments, one siRNA or sdRNA targets TIM3 and one siRNA or sdRNA targets PD-1. In some embodiments, one siRNA or sdRNA targets TIM3 and one siRNA or sdRNA targets LAG3. In some embodiments, one siRNA or sdRNA targets TIM3 and one siRNA or sdRNA targets CISH. In some embodiments, one siRNA or sdRNA targets TIM3 and one siRNA or sdRNA targets CBLB. In some embodiments, one siRNA or sdRNA targets TIM3 and one siRNA or sdRNA targets CTLA-4. In some embodiments, one siRNA or sdRNA targets TIM3 and one siRNA or sdRNA targets TIGIT. In some embodiments, one siRNA or sdRNA targets TIM3 and one siRNA or sdRNA targets TET2. In some embodiments, one siRNA or sdRNA targets CTLA-4 and one siRNA or sdRNA targets TIGIT. In some embodiments, one siRNA or sdRNA targets CTLA-4 and one siRNA or sdRNA targets TET2. In some embodiments, one siRNA or sdRNA targets TIGIT and one siRNA or sdRNA targets TET2.

As discussed herein, embodiments of the present invention provide tumor infiltrating lymphocytes (TILs) that have been genetically modified via gene-editing to enhance their therapeutic effect. Embodiments of the present invention embrace genetic editing through nucleotide insertion (RNA or DNA) into a population of TILs for both promotion of the expression of one or more proteins and inhibition of the expression of one or more proteins, as well as combinations thereof. Embodiments of the present invention also provide methods for expanding TILs into a therapeutic population, wherein the methods comprise gene-editing the TILs. There are several gene-editing technologies that may be used to genetically modify a population of TILs, which are suitable for use in accordance with the present invention. Such methods include the methods described below as well as the viral and transposon methods described elsewhere herein. In some embodiments, a method of genetically modifying a TIL, MIL, or PBL to express a CCR may also include a modification to suppress the expression of a gene either via stable knockout of such a gene or transient knockdown of such a gene.

In some embodiments, the method comprises a method of genetically modifying a population of TILs in a first population, a second population and/or a third population as described herein. In some embodiments, a method of genetically modifying a population of TILs includes the step of stable incorporation of genes for production or inhibition (e.g., silencing) of one more proteins. In some embodiments, a method of genetically modifying a population of TILs includes the step of electroporation. Electroporation methods are known in the art and are described, e.g., in Tsong, Biophys. J. 1991, 60, 297-306, and U.S. Patent Application Publication No. 2014/0227237 A1, the disclosures of each of which are incorporated by reference herein. Other electroporation methods known in the art, such as those described in U.S. Pat. Nos. 5,019,034; 5,128,257; 5,137,817; 5,173,158; 5,232,856; 5,273,525; 5,304,120; 5,318,514; 6,010,613 and 6,078,490, the disclosures of which are incorporated by reference herein, may be used. In some embodiments, the electroporation method is a sterile electroporation method. In some embodiments, the electroporation method is a pulsed electroporation method. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse amplitude. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein at least two of the at least three pulses differ from each other in pulse width. In some embodiments, the electroporation method is a pulsed electroporation method comprising the steps of treating TILs with pulsed electrical fields to alter, manipulate, or cause defined and controlled, permanent or temporary changes in the TILs, comprising the step of applying a sequence of at least three single, operator-controlled, independently programmed, DC electrical pulses, having field strengths equal to or greater than 100 V/cm, to the TILs, wherein a first pulse interval for a first set of two of the at least three pulses is different from a second pulse interval for a second set of two of the at least three pulses. In some embodiments, the electroporation method is a pulsed electroporation method comprising the steps of treating TILs with pulsed electrical fields to induce pore formation in the TILs, comprising the step of applying a sequence of at least three DC electrical pulses, having field strengths equal to or greater than 100 V/cm, to TILs, wherein the sequence of at least three DC electrical pulses has one, two, or three of the following characteristics: (1) at least two of the at least three pulses differ from each other in pulse amplitude; (2) at least two of the at least three pulses differ from each other in pulse width; and (3) a first pulse interval for a first set of two of the at least three pulses is different from a second pulse interval for a second set of two of the at least three pulses, such that induced pores are sustained for a relatively long period of time, and such that viability of the TILs is maintained. In some embodiments, a method of genetically modifying a population of TILs includes the step of calcium phosphate transfection. Calcium phosphate transfection methods (calcium phosphate DNA precipitation, cell surface coating, and endocytosis) are known in the art and are described in Graham and van der Eb, Virology 1973, 52, 456-467; Wigler, et al., Proc. Natl. Acad. Sci. 1979, 76, 1373-1376; and Chen and Okayarea, Mol. Cell. Biol. 1987, 7, 2745-2752; and in U.S. Pat. No. 5,593,875, the disclosures of each of which are incorporated by reference herein. In some embodiments, a method of genetically modifying a population of TILs includes the step of liposomal transfection. Liposomal transfection methods, such as methods that employ a 1:1 (w/w) liposome formulation of the cationic lipid N-[1-(2,3-dioleyloxy)propyl]-n,n,n-trimethylammonium chloride (DOTMA) and dioleoyl phophotidylethanolamine (DOPE) in filtered water, are known in the art and are described in Rose, et al., Biotechniques 1991, 10, 520-525 and Felgner, et al., Proc. Natl. Acad. Sci. USA, 1987, 84, 7413-7417 and in U.S. Pat. Nos. 5,279,833; 5,908,635; 6,056,938; 6,110,490; 6,534,484; and 7,687,070, the disclosures of each of which are incorporated by reference herein. In some embodiments, a method of genetically modifying a population of TILs includes the step of transfection using methods described in U.S. Pat. Nos. 5,766,902; 6,025,337; 6,410,517; 6,475,994; and 7,189,705; the disclosures of each of which are incorporated by reference herein. The TILs may be a first population, a second population and/or a third population of TILs as described herein.

According to an embodiment, the gene-editing process may comprise the use of a programmable nuclease that mediates the generation of a double-strand or single-strand break at one or more immune checkpoint genes. Such programmable nucleases enable precise genome editing by introducing breaks at specific genomic loci, i.e., they rely on the recognition of a specific DNA sequence within the genome to target a nuclease domain to this location and mediate the generation of a double-strand break at the target sequence. A double-strand break in the DNA subsequently recruits endogenous repair machinery to the break site to mediate genome editing by either non-homologous end-joining (NHEJ) or homology-directed repair (HDR). Thus, the repair of the break can result in the introduction of insertion/deletion mutations that disrupt (e.g., silence, repress, or enhance) the target gene product.

Major classes of nucleases that have been developed to enable site-specific genomic editing include zinc finger nucleases (ZFNs), transcription activator-like nucleases (TALENs), and CRISPR-associated nucleases (e.g., CRISPR/Cas9). These nuclease systems can be broadly classified into two categories based on their mode of DNA recognition: ZFNs and TALENs achieve specific DNA binding via protein-DNA interactions, whereas CRISPR systems, such as Cas9, are targeted to specific DNA sequences by a short RNA guide molecule that base-pairs directly with the target DNA and by protein-DNA interactions. See, e.g., Cox et al., Nature Medicine, 2015, Vol. 21, No. 2.

Non-limiting examples of gene-editing methods that may be used in accordance with TIL expansion methods of the present invention include CRISPR methods, TALE methods, and ZFN methods, which are described in more detail below. According to an embodiment, a method for expanding TILs into a therapeutic population may be carried out in accordance with any embodiment of the methods described herein (e.g., Gen 2) or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by one or more of a CRISPR method, a TALE method or a ZFN method, in order to generate TILs that can provide an enhanced therapeutic effect. According to an embodiment, gene-edited TILs can be evaluated for an improved therapeutic effect by comparing them to non-modified TILs in vitro, e.g., by evaluating in vitro effector function, cytokine profiles, etc. compared to unmodified TILs. In certain embodiments, the method comprises gene editing a population of TILs using CRISPR, TALE and/or ZFN methods.

In some embodiments of the present invention, electroporation is used for delivery of a gene editing system, such as CRISPR, TALEN, and ZFN systems. In some embodiments of the present invention, the electroporation system is a flow electroporation system. An example of a suitable flow electroporation system suitable for use with some embodiments of the present invention is the commercially-available MaxCyte STX system. There are several alternative commercially-available electroporation instruments which may be suitable for use with the present invention, such as the AgilePulse system or ECM 830 available from BTX-Harvard Apparatus, Cellaxess Elektra (Cellectricon), Nucleofector (Lonza/Amaxa), GenePulser MXcell (BIORAD), iPorator-96 (Primax) or siPORTer96 (Ambion). In some embodiments of the present invention, the electroporation system forms a closed, sterile system with the remainder of the TIL expansion method. In some embodiments of the present invention, the electroporation system is a pulsed electroporation system as described herein, and forms a closed, sterile system with the remainder of the TIL expansion method.

A method for expanding TILs into a therapeutic population may be carried out in accordance with any embodiment of the methods described herein (e.g., Gen 2) or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by a CRISPR method (e.g., CRISPR/Cas9 or CRISPR/Cpf1). According to particular embodiments, the use of a CRISPR method during the TIL expansion process causes expression of one or more immune checkpoint genes to be silenced or reduced in at least a portion of the therapeutic population of TILs. Alternatively, the use of a CRISPR method during the TIL expansion process causes expression of one or more immune checkpoint genes to be enhanced in at least a portion of the therapeutic population of TILs.

CRISPR stands for clustered regularly interspaced short palindromic repeats. A method of using a CRISPR system for gene editing is also referred to herein as a CRISPR method. There are three types of CRISPR systems which incorporate RNAs and Cas proteins, and which may be used in accordance with the present invention: Types I, II, and III. The Type II CRISPR (exemplified by Cas9) is one of the most well-characterized systems.

CRISPR technology was adapted from the natural defense mechanisms of bacteria and archaea (the domain of single-celled microorganisms). These organisms use CRISPR-derived RNA and various Cas proteins, including Cas9, to foil attacks by viruses and other foreign bodies by chopping up and destroying the DNA of a foreign invader. A CRISPR is a specialized region of DNA with two distinct characteristics: the presence of nucleotide repeats and spacers. Repeated sequences of nucleotides are distributed throughout a CRISPR region with short segments of foreign DNA (spacers) interspersed among the repeated sequences. In the type II CRISPR/Cas system, spacers are integrated within the CRISPR genomic loci and transcribed and processed into short CRISPR RNA (crRNA). These crRNAs anneal to trans-activating crRNAs (tracrRNAs) and direct sequence-specific cleavage and silencing of pathogenic DNA by Cas proteins. Target recognition by the Cas9 protein requires a “seed” sequence within the crRNA and a conserved dinucleotide-containing protospacer adjacent motif (PAM) sequence upstream of the crRNA-binding region. The CRISPR/Cas system can thereby be retargeted to cleave virtually any DNA sequence by redesigning the crRNA. The crRNA and tracrRNA in the native system can be simplified into a single guide RNA (sgRNA) of approximately 100 nucleotides for use in genetic engineering. The CRISPR/Cas system is directly portable to human cells by co-delivery of plasmids expressing the Cas9 endo-nuclease and the necessary crRNA components. Different variants of Cas proteins may be used to reduce targeting limitations (e.g., orthologs of Cas9, such as Cpf1).

Non-limiting examples of genes that may be silenced or inhibited by permanently gene-editing TILs via a CRISPR method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, and BCOR.

Non-limiting examples of genes that may be enhanced by permanently gene-editing TILs via a CRISPR method include CCR2, CCR4, CCR5, CXCR2, CXCR3, CX3CR1, IL-2, IL12, IL-15, IL-18 and IL-21.

Examples of systems, methods, and compositions for altering the expression of a target gene sequence by a CRISPR method, and which may be used in accordance with embodiments of the present invention, are described in U.S. Pat. Nos. 8,697,359; 8,993,233; 8,795,965; 8,771,945; 8,889,356; 8,865,406; 8,999,641; 8,945,839; 8,932,814; 8,871,445; 8,906,616; and 8,895,308, the disclosures of each of which are incorporated by reference herein. Resources for carrying out CRISPR methods, such as plasmids for expressing CRISPR/Cas9 and CRISPR/Cpf1, are commercially available from companies such as GenScript.

In some embodiments, genetic modifications of populations of TILs, as described herein, may be performed using the CRISPR/Cpf1 system as described in U.S. Pat. No. 9,790,490, the disclosure of which is incorporated by reference herein.

A method for expanding TILs into a therapeutic population may be carried out in accordance with any embodiment of the methods described herein (e.g., Gen 2) or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by a TALE method. According to particular embodiments, the use of a TALE method during the TIL expansion process causes expression of one or more immune checkpoint genes to be silenced or reduced in at least a portion of the therapeutic population of TILs. Alternatively, the use of a TALE method during the TIL expansion process causes expression of one or more immune checkpoint genes to be enhanced in at least a portion of the therapeutic population of TILs.

TALE stands for transcription activator-like effector proteins, which include transcription activator-like effector nucleases (TALENs). A method of using a TALE system for gene editing may also be referred to herein as a TALE method. TALEs are naturally occurring proteins from the plant pathogenic bacteria genus Xanthomonas, and contain DNA-binding domains composed of a series of 33-35-amino-acid repeat domains that each recognizes a single base pair. TALE specificity is determined by two hypervariable amino acids that are known as the repeat-variable di-residues (RVDs). Modular TALE repeats are linked together to recognize contiguous DNA sequences. A specific RVD in the DNA-binding domain recognizes a base in the target locus, providing a structural feature to assemble predictable DNA-binding domains. The DNA binding domains of a TALE are fused to the catalytic domain of a type IIS FokI endonuclease to make a targetable TALE nuclease. To induce site-specific mutation, two individual TALEN arms, separated by a 14-20 base pair spacer region, bring FokI monomers in close proximity to dimerize and produce a targeted double-strand break.

Several large, systematic studies utilizing various assembly methods have indicated that TALE repeats can be combined to recognize virtually any user-defined sequence. Custom-designed TALE arrays are also commercially available through Cellectis Bioresearch (Paris, France), Transposagen Biopharmaceuticals (Lexington, KY, USA), and Life Technologies (Grand Island, NY, USA). TALE and TALEN methods suitable for use in the present invention are described in U.S. Patent Application Publication Nos. US 2011/0201118 A1; US 2013/0117869 A1; US 2013/0315884 A1; US 2015/0203871 A1 and US 2016/0120906 A1, the disclosures of each of which are incorporated by reference herein.

Non-limiting examples of genes that may be silenced or inhibited by permanently gene-editing TILs via a TALE method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, and BCOR.

Non-limiting examples of genes that may be enhanced by permanently gene-editing TILs via a TALE method include CCR2, CCR4, CCR5, CXCR2, CXCR3, CX3CR1, IL-2, IL12, IL-15, IL-18 and IL-21.

Examples of systems, methods, and compositions for altering the expression of a target gene sequence by a TALE method, and which may be used in accordance with embodiments of the present invention, are described in U.S. Pat. No. 8,586,526, which is incorporated by reference herein.

A method for expanding TILs into a therapeutic population may be carried out in accordance with any embodiment of the methods described herein or as described in U.S. Patent Application Publication Nos. US 2020/0299644 A1 and US 2020/0121719 A1 and U.S. Pat. No. 10,925,900, the disclosures of which are incorporated by reference herein, wherein the method further comprises gene-editing at least a portion of the TILs by a zinc finger or zinc finger nuclease method. According to particular embodiments, the use of a zinc finger method during the TIL expansion process causes expression of one or more immune checkpoint genes to be silenced or reduced in at least a portion of the therapeutic population of TILs. Alternatively, the use of a zinc finger method during the TIL expansion process causes expression of one or more immune checkpoint genes to be enhanced in at least a portion of the therapeutic population of TILs.

An individual zinc finger contains approximately 30 amino acids in a conserved 00a configuration. Several amino acids on the surface of the α-helix typically contact 3 bp in the major groove of DNA, with varying levels of selectivity. Zinc fingers have two protein domains. The first domain is the DNA binding domain, which includes eukaryotic transcription factors and contain the zinc finger. The second domain is the nuclease domain, which includes the FokI restriction enzyme and is responsible for the catalytic cleavage of DNA.

The DNA-binding domains of individual ZFNs typically contain between three and six individual zinc finger repeats and can each recognize between 9 and 18 base pairs. If the zinc finger domains are specific for their intended target site then even a pair of 3-finger ZFNs that recognize a total of 18 base pairs can, in theory, target a single locus in a mammalian genome. One method to generate new zinc-finger arrays is to combine smaller zinc-finger “modules” of known specificity. The most common modular assembly process involves combining three separate zinc fingers that can each recognize a 3 base pair DNA sequence to generate a 3-finger array that can recognize a 9 base pair target site. Alternatively, selection-based approaches, such as oligomerized pool engineering (OPEN) can be used to select for new zinc-finger arrays from randomized libraries that take into consideration context-dependent interactions between neighboring fingers. Engineered zinc fingers are available commercially from Sangamo Biosciences (Richmond, CA, USA) and Sigma-Aldrich (St. Louis, MO, USA).

Non-limiting examples of genes that may be silenced or inhibited by permanently gene-editing TILs via a zinc finger method include PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, and BCOR.

Non-limiting examples of genes that may be enhanced by permanently gene-editing TILs via a zinc finger method include CCR2, CCR4, CCR5, CXCR2, CXCR3, CX3CR1, IL-2, IL12, IL-15, IL-18 and IL-21.

Examples of systems, methods, and compositions for altering the expression of a target gene sequence by a zinc finger method, which may be used in accordance with embodiments of the present invention, are described in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, each of which are incorporated by reference herein.

Other examples of systems, methods, and compositions for altering the expression of a target gene sequence by a zinc finger method, which may be used in accordance with embodiments of the present invention, are described in Beane, et al., Mol. Therapy, 2015, 23, 1380-1390, the disclosure of which is incorporated by reference herein.

In some embodiments, the TILs are optionally genetically engineered to include additional functionalities, including, but not limited to, a high-affinity TCR, e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19). In certain embodiments, the method comprises genetically engineering a population of TILs to include a high-affinity TCR, e.g., a TCR targeted at a tumor-associated antigen such as MAGE-1, HER2, or NY-ESO-1, or a chimeric antigen receptor (CAR) which binds to a tumor-associated cell surface molecule (e.g., mesothelin) or lineage-restricted cell surface molecule (e.g., CD19). Aptly, the population of TILs may be a first population, a second population and/or a third population as described herein.

### D. Closed Systems for TIL Manufacturing

The present invention provides for the use of closed systems during the TIL culturing process. Such closed systems allow for preventing and/or reducing microbial contamination, allow for the use of fewer flasks, and allow for cost reductions. In some embodiments, the closed system uses two containers.

Such closed systems are well-known in the art and can be found, for example, at http://www.fda.gov/cber/guidelines.htm and https://www.fda.gov/BiologicsBloodVaccines/GuidanceComplianceRegulatorylnformation/Guidances/Blood/ucm076779.htm.

Sterile connecting devices (STCDs) produce sterile welds between two pieces of compatible tubing. This procedure permits sterile connection of a variety of containers and tube diameters. In some embodiments, the closed systems include luer lock and heat-sealed systems as described in the Examples. In some embodiments, the closed system is accessed via syringes under sterile conditions in order to maintain the sterility and closed nature of the system. In some embodiments, a closed system as described in the examples is employed. In some embodiments, the TILs are formulated into a final product formulation container according to the methods described herein in the examples.

In some embodiments, the closed system uses one container from the time the tumor fragments are obtained until the TILs are ready for administration to the patient or cryopreserving. In some embodiments when two containers are used, the first container is a closed G-container and the population of TILs is centrifuged and transferred to an infusion bag without opening the first closed G-container. In some embodiments, when two containers are used, the infusion bag is a HypoThermosol-containing infusion bag. A closed system or closed TIL cell culture system is characterized in that once the tumor sample and/or tumor fragments have been added, the system is tightly sealed from the outside to form a closed environment free from the invasion of bacteria, fungi, and/or any other microbial contamination.

In some embodiments, the reduction in microbial contamination is between about 5% and about 100%. In some embodiments, the reduction in microbial contamination is between about 5% and about 95%. In some embodiments, the reduction in microbial contamination is between about 5% and about 90%. In some embodiments, the reduction in microbial contamination is between about 10% and about 90%. In some embodiments, the reduction in microbial contamination is between about 15% and about 85%. In some embodiments, the reduction in microbial contamination is about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 97%, about 98%, about 99%, or about 100%.

The closed system allows for TIL growth in the absence and/or with a significant reduction in microbial contamination.

Moreover, pH, carbon dioxide partial pressure and oxygen partial pressure of the TIL cell culture environment each vary as the cells are cultured. Consequently, even though a medium appropriate for cell culture is circulated, the closed environment still needs to be constantly maintained as an optimal environment for TIL proliferation. To this end, it is desirable that the physical factors of pH, carbon dioxide partial pressure and oxygen partial pressure within the culture liquid of the closed environment be monitored by means of a sensor, the signal whereof is used to control a gas exchanger installed at the inlet of the culture environment, and the that gas partial pressure of the closed environment be adjusted in real time according to changes in the culture liquid so as to optimize the cell culture environment. In some embodiments, the present invention provides a closed cell culture system which incorporates at the inlet to the closed environment a gas exchanger equipped with a monitoring device which measures the pH, carbon dioxide partial pressure and oxygen partial pressure of the closed environment, and optimizes the cell culture environment by automatically adjusting gas concentrations based on signals from the monitoring device.

In some embodiments, the pressure within the closed environment is continuously or intermittently controlled. That is, the pressure in the closed environment can be varied by means of a pressure maintenance device for example, thus ensuring that the space is suitable for growth of TILs in a positive pressure state, or promoting exudation of fluid in a negative pressure state and thus promoting cell proliferation. By applying negative pressure intermittently, moreover, it is possible to uniformly and efficiently replace the circulating liquid in the closed environment by means of a temporary shrinkage in the volume of the closed environment.

In some embodiments, optimal culture components for proliferation of the TILs can be substituted or added, and including factors such as IL-2 and/or OKT3, as well as combination, can be added.

### E. Optional Cryopreservation of TILs

Either the bulk TIL population (for example the second population of TILs) or the expanded population of TILs (for example the third population of TILs) can be optionally cryopreserved. In some embodiments, cryopreservation occurs on the therapeutic TIL population. In some embodiments, cryopreservation occurs on the TILs harvested after the second expansion. In some embodiments, cryopreservation occurs on the TILs in exemplary Step F of FIGS. 8 (in particular, e.g., FIG. 8A and/or FIG. 8B and/or FIG. 8C and/or FIG. 8D). In some embodiments, the TILs are cryopreserved in the infusion bag. In some embodiments, the TILs are cryopreserved prior to placement in an infusion bag. In some embodiments, the TILs are cryopreserved and not placed in an infusion bag. In some embodiments, cryopreservation is performed using a cryopreservation medium. In some embodiments, the cryopreservation media contains dimethylsulfoxide (DMSO). This is generally accomplished by putting the TIL population into a freezing solution, e.g. 85% complement inactivated AB serum and 15% dimethyl sulfoxide (DMSO). The cells in solution are placed into cryogenic vials and stored for 24 hours at −80° C., with optional transfer to gaseous nitrogen freezers for cryopreservation. See, Sadeghi, et al., Acta Oncologica 2013, 52, 978-986.

When appropriate, the cells are removed from the freezer and thawed in a 37° C. water bath until approximately 4/5 of the solution is thawed. The cells are generally resuspended in complete media and optionally washed one or more times. In some embodiments, the thawed TILs can be counted and assessed for viability as is known in the art.

In some embodiments, a population of TILs is cryopreserved using CS10 cryopreservation media (CryoStor 10, BioLife Solutions). In some embodiments, a population of TILs is cryopreserved using a cryopreservation media containing dimethylsulfoxide (DMSO). In some embodiments, a population of TILs is cryopreserved using a 1:1 (vol:vol) ratio of CS10 and cell culture media. In some embodiments, a population of TILs is cryopreserved using about a 1:1 (vol:vol) ratio of CS10 and cell culture media, further comprising additional IL-2.

As discussed above, and exemplified in Steps A through E as provided in FIGS. 1 and/or 8 (in particular, e.g., FIG. 8A and/or FIG. 8B and/or FIG. 8C and/or FIG. 8D), cryopreservation can occur at numerous points throughout the TIL expansion process. In some embodiments, the expanded population of TILs after the first expansion (as provided for example, according to Step B or the expanded population of TILs after the one or more second expansions according to Step D of FIG. 1 or 8 (in particular, e.g., FIG. 8A and/or FIG. 8B and/or FIG. 8C and/or FIG. 8D) can be cryopreserved. Cryopreservation can be generally accomplished by placing the TIL population into a freezing solution, e.g., 85% complement inactivated AB serum and 15% dimethyl sulfoxide (DMSO). The cells in solution are placed into cryogenic vials and stored for 24 hours at −80° C., with optional transfer to gaseous nitrogen freezers for cryopreservation. See Sadeghi, et al., Acta Oncologica 2013, 52, 978-986. In some embodiments, the TILs are cryopreserved in 5% DMSO. In some embodiments, the TILs are cryopreserved in cell culture media plus 5% DMSO. In some embodiments, the TILs are cryopreserved according to the methods provided in Example 6.

When appropriate, the cells are removed from the freezer and thawed in a 37° C. water bath until approximately 4/5 of the solution is thawed. The cells are generally resuspended in complete media and optionally washed one or more times. In some embodiments, the thawed TILs can be counted and assessed for viability as is known in the art.

In some cases, the Step B from FIG. 1 or 8, (in particular, e.g., FIG. 8A and/or FIG. 8B and/or FIG. 8C and/or FIG. 8D) TIL population can be cryopreserved immediately, using the protocols discussed below. Alternatively, the bulk TIL population can be subjected to Step C and Step D from FIG. 1 or 8, (in particular, e.g., FIG. 8A and/or FIG. 8B and/or FIG. 8C and/or FIG. 8D) and then cryopreserved after Step D from FIG. 1 or 8, (in particular, e.g., FIG. 8A and/or FIG. 8B and/or FIG. 8C and/or FIG. 8D). Similarly, in the case where genetically modified TILs will be used in therapy, the Step B or Step D from FIG. 1 or 8, (in particular, e.g., FIG. 8A and/or FIG. 8B and/or FIG. 8C and/or FIG. 8D) TIL populations can be subjected to genetic modifications for suitable treatments.

### F. Phenotypic Characteristics of Expanded TILs

In some embodiments, the TILs are analyzed for expression of numerous phenotype markers after expansion, including those described herein and in the Examples. In some embodiments, expression of one or more phenotypic markers is examined. In some embodiments, the phenotypic characteristics of the TILs are analyzed after the first expansion in Step B from FIG. 1 or 8, (in particular, e.g., FIG. 8A and/or FIG. 8B and/or FIG. 8C and/or FIG. 8D). In some embodiments, the phenotypic characteristics of the TILs are analyzed during the transition in Step C from FIG. 1 or 8, (in particular, e.g., FIG. 8A and/or FIG. 8B and/or FIG. 8C and/or FIG. 8D). In some embodiments, the phenotypic characteristics of the TILs are analyzed during the transition according to Step C from FIG. 1 or 8, (in particular, e.g., FIG. 8A and/or FIG. 8B and/or FIG. 8C and/or FIG. 8D) and after cryopreservation. In some embodiments, the phenotypic characteristics of the TILs are analyzed after the second expansion according to Step D from FIG. 1 or 8, (in particular, e.g., FIG. 8A and/or FIG. 8B and/or FIG. 8C and/or FIG. 8D). In some embodiments, the phenotypic characteristics of the TILs are analyzed after two or more expansions according to Step D from FIG. 1 or 8, (in particular, e.g., FIG. 8A and/or FIG. 8B and/or FIG. 8C and/or FIG. 8D).

In some embodiments, the marker is selected from the group consisting of CD8 and CD28. In some embodiments, expression of CD8 is examined. In some embodiments, expression of CD28 is examined. In some embodiments, the expression of CD8 and/or CD28 is higher on TILs produced according the current invention process, as compared to other processes (e.g., the Gen 3 process as provided for example in FIG. 8 (in particular, e.g., FIG. 8A and/or FIG. 8B and/or FIG. 8C and/or FIG. 8D), as compared to the 2A process as provided for example in FIG. 8 (in particular, e.g., FIG. 8A and/or FIG. 8B and/or FIG. 8C and/or FIG. 8D). In some embodiments, the expression of CD8 is higher on TILs produced according the current invention process, as compared to other processes (e.g., the Gen 3 process as provided for example in FIG. 8 (in particular, e.g., FIG. 8B), as compared to the 2A process as provided for example in FIG. 8 (in particular, e.g., FIG. 8A and/or FIG. 8B and/or FIG. 8C and/or FIG. 8D). In some embodiments, the expression of CD28 is higher on TILs produced according the current invention process, as compared to other processes (e.g., the Gen 3 process as provided for example in FIG. 8 (in particular, e.g., FIG. 8A and/or FIG. 8B and/or FIG. 8C and/or FIG. 8D), as compared to the 2A process as provided for example in FIG. 8 (in particular, e.g., FIG. 8A)). In some embodiments, high CD28 expression is indicative of a younger, more persistent TIL phenotype. In some embodiments, expression of one or more regulatory markers is measured.

In some embodiments, no selection of the first population of TILs, second population of TILs, third population of TILs, or harvested TIL population based on CD8 and/or CD28 expression is performed during any of the steps for the method for expanding tumor infiltrating lymphocytes (TILs) described herein.

In some embodiments, the percentage of central memory cells is higher on TILs produced according the current invention process, as compared to other processes (e.g., the Gen 3 process as provided for example in FIG. 8 (in particular, e.g., FIG. 8A and/or FIG. 8B and/or FIG. 8C and/or FIG. 8D), as compared to the 2A process as provided for example in FIG. 8 (in particular, e.g., FIG. 8A)). In some embodiments the memory marker for central memory cells is selected from the group consisting of CCR7 and CD62L.

In some embodiments, the CD4+ and/or CD8+ TIL Memory subsets can be divided into different memory subsets. In some embodiments, the CD4+ and/or CD8+ TILs comprise the naïve (CD45RA+CD62L+) TILs. In some embodiments, the CD4+ and/or CD8+ TILs comprise the central memory (CM; CD45RA−CD62L+) TILs. In some embodiments, the CD4+ and/or CD8+ TILs comprise the effector memory (EM; CD45RA−CD62L−) TILs. In some embodiments, the CD4+ and/or CD8+ TILs comprise the, RA+ effector memory/effector (TEMRA/TEFF; CD45RA+CD62L+) TILs.

In some embodiments, the TILs express one more markers selected from the group consisting of granzyme B, perform, and granulysin. In some embodiments, the TILs express granzyme B. In some embodiments, the TILs express perforin. In some embodiments, the TILs express granulysin.

In some embodiments, restimulated TILs can also be evaluated for cytokine release, using cytokine release assays. In some embodiments, TILs can be evaluated for interferon-α (IFN-γ) secretion. In some embodiments, the IFN-γ secretion is measured by an ELISA assay. In some embodiments, the IFN-γ secretion is measured by an ELISA assay after the rapid second expansion step, after Step D as provided in for example, FIG. 8 (in particular, e.g., FIG. 8A and/or FIG. 8B and/or FIG. 8C and/or FIG. 8D). In some embodiments, TIL health is measured by IFN-gamma (IFN-γ) secretion. In some embodiments, IFN-γ secretion is indicative of active TILs. In some embodiments, a potency assay for IFN-γ production is employed. IFN-γ production is another measure of cytotoxic potential. IFN-γ production can be measured by determining the levels of the cytokine IFN-γ in the media of TIL stimulated with antibodies to CD3, CD28, and CD137/4-1BB. IFN-γ levels in media from these stimulated TIL can be determined using by measuring IFN-γ release. In some embodiments, an increase in IFN-γ production in for example Step D in the Gen 3 process as provided in FIG. 8 (in particular, e.g., FIG. 8A and/or FIG. 8B and/or FIG. 8C and/or FIG. 8D) TILs as compared to for example Step D in the 2A process as provided in FIG. 8 (in particular, e.g., FIG. 8A) is indicative of an increase in cytotoxic potential of the Step D TILs. In some embodiments, IFN-γ secretion is increased one-fold, two-fold, three-fold, four-fold, or five-fold or more. In some embodiments, IFN-γ secretion is increased one-fold. In some embodiments, IFN-γ secretion is increased two-fold. In some embodiments, IFN-γ secretion is increased three-fold. In some embodiments, IFN-γ secretion is increased four-fold. In some embodiments, IFN-γ secretion is increased five-fold. In some embodiments, IFN-γ is measured using a Quantikine ELISA kit. In some embodiments, IFN-γ is measured in TILs ex vivo. In some embodiments, IFN-γ is measured in TILs ex vivo, including TILs produced by the methods of the present invention, including, for example FIG. 8B methods.

In some embodiments, TILs capable of at least one-fold, two-fold, three-fold, four-fold, or five-fold or more IFN-γ secretion are TILs produced by the expansion methods of the present invention, including, for example FIG. 8A and/or FIG. 8B and/or FIG. 8C and/or FIG. 8D methods. In some embodiments, TILs capable of at least one-fold more IFN-γ secretion are TILs produced by the expansion methods of the present invention, including, for example FIG. 8A and/or FIG. 8B and/or FIG. 8C and/or FIG. 8D methods. In some embodiments, TILs capable of at least two-fold more IFN-γ secretion are TILs produced by the expansion methods of the present invention, including, for example FIG. 8A and/or FIG. 8B and/or FIG. 8C and/or FIG. 8D methods. In some embodiments, TILs capable of at least three-fold more IFN-γ secretion are TILs produced by the expansion methods of the present invention, including, for example FIG. 8A and/or FIG. 8B and/or FIG. 8C and/or FIG. 8D methods. In some embodiments, TILs capable of at least four-fold more IFN-γ secretion are TILs produced by the expansion methods of the present invention, including, for example FIG. 8A and/or FIG. 8B and/or FIG. 8C and/or FIG. 8D methods. In some embodiments, TILs capable of at least five-fold more IFN-γ secretion are TILs produced by the expansion methods of the present invention, including, for example FIG. 8A and/or FIG. 8B and/or FIG. 8C and/or FIG. 8D methods.

In some embodiments, TILs capable of at least 100 μg/mL to about 1000 pg/mL or more IFN-γ secretion are TILs produced by the expansion methods of the present invention, including, for example FIG. 8A and/or FIG. 8B and/or FIG. 8C and/or FIG. 8D methods. In some embodiments, TILs capable of at least 200 pg/mL, at least 250 pg/mL, at least 300 pg/mL, at least 350 pg/mL, at least 400 pg/mL, at least 450 pg/mL, at least 500 pg/mL, at least 550 pg/mL, at least 600 pg/mL, at least 650 pg/mL, at least 700 pg/mL, at least 750 pg/mL, at least 800 pg/mL, at least 850 pg/mL, at least 900 pg/mL, at least 950 pg/mL, or at least 1000 pg/mL or more IFN-γ secretion are TILs produced by the expansion methods of the present invention, including, for example FIG. 8A and/or FIG. 8B and/or FIG. 8C and/or FIG. 8D methods. In some embodiments, TILs capable of at least 200 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 200 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 300 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 400 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 500 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 600 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 700 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 800 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 900 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 1000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 2000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 3000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 4000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 5000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 6000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 7000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 8000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 9000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 10,000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 15,000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 20,000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 25,000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 30,000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 35,000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 40,000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 45,000 pg/mL IFN-γ secretion are TILs produced by the expansion methods of the present invention, including, for example FIG. 8A and/or FIG. 8B and/or FIG. 8C and/or FIG. 8D methods. In some embodiments, TILs capable of at least 50,000 pg/mL IFN-γ secretion are TILs produced by the expansion methods of the present invention, including, for example FIG. 8A and/or FIG. 8B and/or FIG. 8C and/or FIG. 8D methods.

In some embodiments, TILs capable of at least 100 pg/mL/5e5 cells to about 1000 pg/mL/5e5 cells or more IFN-γ secretion are TILs produced by the expansion methods of the present invention, including, for example FIG. 8A and/or FIG. 8B and/or FIG. 8C and/or FIG. 8D methods. In some embodiments, TILs capable of at least 200 pg/mL/5e5 cells, at least 250 pg/mL/5e5 cells, at least 300 pg/mL/5e5 cells, at least 350 pg/mL/5e5 cells, at least 400 pg/mL/5e5 cells, at least 450 pg/mL/5e5 cells, at least 500 pg/mL/5e5 cells, at least 550 pg/mL/5e5 cells, at least 600 pg/mL/5e5 cells, at least 650 pg/mL/5e5 cells, at least 700 pg/mL/5e5 cells, at least 750 pg/mL/5e5 cells, at least 800 pg/mL/5e5 cells, at least 850 pg/mL/5e5 cells, at least 900 pg/mL/5e5 cells, at least 950 pg/mL/5e5 cells, or at least 1000 pg/mL/5e5 cells or more IFN-γ secretion are TILs produced by the expansion methods of the present invention, including, for example FIG. 8A and/or FIG. 8B and/or FIG. 8C and/or FIG. 8D methods. In some embodiments, TILs capable of at least 200 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 200 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 300 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 400 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 500 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 600 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 700 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 800 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 900 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 1000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 2000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 3000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 4000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 5000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 6000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 7000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 8000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 9000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 10,000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 15,000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 20,000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 25,000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 30,000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 35,000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 40,000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 45,000 pg/mL/5e5 cells IFN-γ secretion are TILs produced by the expansion methods of the present invention, including, for example FIG. 8A and/or FIG. 8B and/or FIG. 8C and/or FIG. 8D methods. In some embodiments, TILs capable of at least 50,000 pg/mL/5e5 cells IFN-γ secretion are TILs produced by the expansion methods of the present invention, including, for example FIG. 8A and/or FIG. 8B and/or FIG. 8C and/or FIG. 8D methods.

The diverse antigen receptors of T and B lymphocytes are produced by somatic recombination of a limited, but large number of gene segments. These gene segments: V (variable), D (diversity), J (joining), and C (constant), determine the binding specificity and downstream applications of immunoglobulins and T-cell receptors (TCRs). The present invention provides a method for generating TILs which exhibit and increase the T-cell repertoire diversity. In some embodiments, the TILs obtained by the present method exhibit an increase in the T-cell repertoire diversity. In some embodiments, the TILs obtained by the present method exhibit an increase in the T-cell repertoire diversity as compared to freshly harvested TILs and/or TILs prepared using other methods than those provide herein including, for example, methods other than those embodied in FIG. 8 (in particular, e.g., FIG. 8A and/or FIG. 8B and/or FIG. 8C and/or FIG. 8D). In some embodiments, the TILs obtained by the present method exhibit an increase in the T-cell repertoire diversity as compared to freshly harvested TILs and/or TILs prepared using methods referred to as Gen 2, as exemplified in FIG. 8 (in particular, e.g., FIG. 8A). In some embodiments, the TILs obtained in the first expansion exhibit an increase in the T-cell repertoire diversity. In some embodiments, the increase in diversity is an increase in the immunoglobulin diversity and/or the T-cell receptor diversity. In some embodiments, the diversity is in the immunoglobulin is in the immunoglobulin heavy chain. In some embodiments, the diversity is in the immunoglobulin is in the immunoglobulin light chain. In some embodiments, the diversity is in the T-cell receptor. In some embodiments, the diversity is in one of the T-cell receptors selected from the group consisting of alpha, beta, gamma, and delta receptors. In some embodiments, there is an increase in the expression of T-cell receptor (TCR) alpha and/or beta. In some embodiments, there is an increase in the expression of T-cell receptor (TCR) alpha. In some embodiments, there is an increase in the expression of T-cell receptor (TCR) beta. In some embodiments, there is an increase in the expression of TCRab (i.e., TCRX/D). In some embodiments, the process as described herein (e.g., the Gen 3 process) shows higher clonal diversity as compared to other processes, for example the process referred to as the Gen 2 based on the number of unique peptide CDRs within the sample.

In some embodiments, the activation and exhaustion of TILs can be determined by examining one or more markers. In some embodiments, the activation and exhaustion can be determined using multicolor flow cytometry. In some embodiments, the activation and exhaustion of markers include but not limited to one or more markers selected from the group consisting of CD3, PD-1, 2B4/CD244, CD8, CD25, BTLA, KLRG, TIM-3, CD194/CCR4, CD4, TIGIT, CD183, CD69, CD95, CD127, CD103, and/or LAG-3). In some embodiments, the activation and exhaustion of markers include but not limited to one or more markers selected from the group consisting of BTLA, CTLA-4, ICOS, Ki67, LAG-3, PD-1, TIGIT, and/or TIM-3. In some embodiments, the activation and exhaustion of markers include but not limited to one or more markers selected from the group consisting of BTLA, CTLA-4, ICOS, Ki67, LAG-3, CD103+/CD69+, CD103+/CD69−, PD-1, TIGIT, and/or TIM-3. In some embodiments, the T-cell markers (including activation and exhaustion markers) can be determined and/or analyzed to examine T-cell activation, inhibition, or function. In some embodiments, the T-cell markers can include but are not limited to one or more markers selected from the group consisting of TIGIT, CD3, FoxP3, Tim-3, PD-1, CD103, CTLA-4, LAG-3, BTLA-4, ICOS, Ki67, CD8, CD25, CD45, CD4, and/or CD59.

In some embodiments, TILs that exhibit greater than 3000 pg/10⁶ TILs to 300000 pg/10⁶ TILs or more Granzyme B secretion are TILs produced by the expansion methods of the present invention, including for example FIG. 8A and/or FIG. 8B and/or FIG. 8C and/or FIG. 8D. In some embodiments, TILs that exhibit greater than 3000 pg/10⁶ TILs greater than 5000 pg/10⁶ TILs, greater than 7000 pg/10⁶ TILs, greater than 9000 pg/10⁶ TILs, greater than 11000 pg/10⁶ TILs, greater than 13000 pg/10⁶ TILs, greater than 15000 pg/10⁶ TILs, greater than 17000 pg/10⁶ TILs, greater than 19000 pg/10⁶ TILs, greater than 20000 pg/10⁶ TILs, greater than 40000 pg/10⁶ TILs, greater than 60000 pg/10⁶ TILs, greater than 80000 pg/10⁶ TILs, greater than 100000 pg/10⁶ TILs, greater than 120000 pg/10⁶ TILs, greater than 140000 pg/10⁶ TILs, greater than 160000 pg/10⁶ TILs, greater than 180000 pg/10⁶ TILs, greater than 200000 pg/10⁶ TILs, greater than 220000 pg/10⁶ TILs, greater than 240000 pg/10⁶ TILs, greater than 260000 pg/10⁶ TILs, greater than 280000 pg/10⁶ TILs, greater than 300000 pg/10⁶ TILs or more Granzyme B secretion are TILs produced by the expansion methods of the present invention, including for example FIG. 8A and/or FIG. 8B and/or FIG. 8C and/or FIG. 8D. In some embodiments, TILs that exhibit greater than 3000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 5000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 7000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 9000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 11000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 13000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 15000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 17000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 19000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 20000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 40000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 60000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 80000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 100000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 120000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 140000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 160000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 180000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 200000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 220000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 240000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 260000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 280000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 300000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 3000 pg/10⁶ TILs to 300000 pg/10⁶ TILs or more Granzyme B secretion are TILs produced by the expansion methods of the present invention, including for example FIG. 8A and/or FIG. 8B and/or FIG. 8C and/or FIG. 8D. In some embodiments, TILs that exhibit greater than 3000 pg/10⁶ TILs greater than 5000 pg/10⁶ TILs, greater than 7000 pg/10⁶ TILs, greater than 9000 pg/10⁶ TILs, greater than 11000 pg/10⁶ TILs, greater than 13000 pg/10⁶ TILs, greater than 15000 pg/10⁶ TILs, greater than 17000 pg/10⁶ TILs, greater than 19000 pg/10⁶ TILs, greater than 20000 pg/10⁶ TILs, greater than 40000 pg/10⁶ TILs, greater than 60000 pg/10⁶ TILs, greater than 80000 pg/10⁶ TILs, greater than 100000 pg/10⁶ TILs, greater than 120000 pg/10⁶ TILs, greater than 140000 pg/10⁶ TILs, greater than 160000 pg/10⁶ TILs, greater than 180000 pg/10⁶ TILs, greater than 200000 pg/10⁶ TILs, greater than 220000 pg/10⁶ TILs, greater than 240000 pg/10⁶ TILs, greater than 260000 pg/10⁶ TILs, greater than 280000 pg/10⁶ TILs, greater than 300000 pg/10⁶ TILs or more Granzyme B secretion are TILs produced by the expansion methods of the present invention, including for example FIG. 8A and/or FIG. 8B and/or FIG. 8C and/or FIG. 8D. In some embodiments, TILs that exhibit greater than 3000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 5000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 7000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 9000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 11000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 13000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 15000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 17000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 19000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 20000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 40000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 60000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 80000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 100000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 120000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 140000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 160000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 180000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 200000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 220000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 240000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 260000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 280000 pg/10⁶ TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 300000 pg/10⁶ TILs Granzyme B secretion are TILs produced by the expansion methods of the present invention, including for example FIG. 8A and/or FIG. 8B and/or FIG. 8C and/or FIG. 8D.

In some embodiments, TILs that exhibit greater than 1000 pg/mL to 300000 pg/mL or more Granzyme B secretion are TILs produced by the expansion methods of the present invention, including for example FIG. 8A and/or FIG. 8B and/or FIG. 8C and/or FIG. 8D. In some embodiments, TILs that exhibit greater than 1000 pg/mL, greater than 2000 pg/mL, greater than 3000 pg/mL, greater than 4000 pg/mL, greater than 5000 pg/mL, greater than 6000 pg/mL, greater than 7000 pg/mL, greater than 8000 pg/mL, greater than 9000 pg/mL, greater than 10000 pg/mL, greater than 20000 pg/mL, greater than 30000 pg/mL, greater than 40000 pg/mL, greater than 50000 pg/mL, greater than 60000 pg/mL, greater than 70000 pg/mL, greater than 80000 pg/mL, greater than 90000 pg/mL, greater than 100000 pg/mL or more Granzyme B secretion are TILs produced by the expansion methods of the present invention, including for example FIG. 8A and/or FIG. 8B and/or FIG. 8C and/or FIG. 8D. In some embodiments, TILs that exhibit greater than 1000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 2000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 3000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 4000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 5000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 6000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 7000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 8000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 9000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 10000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 20000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 30000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 40000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 50000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 60000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 70000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 80000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 90000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 100000 pg/mL Granzyme B are TILs produced by the expansion methods of the present invention, including for example FIG. 8A and/or FIG. 8B and/or FIG. 8C and/or FIG. 8D. In some embodiments, TILs that exhibit greater than 120000 pg/mL Granzyme B secretion are TILs produced by the expansion methods of the present invention, including for example FIG. 8A and/or FIG. 8B and/or FIG. 8C and/or FIG. 8D. In some embodiments, TILs that exhibit greater than 140000 pg/mL Granzyme B are TILs Granzyme B secretion are TILs produced by the expansion methods of the present invention, including for example FIG. 8A and/or FIG. 8B and/or FIG. 8C and/or FIG. 8D. In some embodiments, TILs that exhibit greater than 160000 pg/mL Granzyme B secretion are TILs produced by the expansion methods of the present invention, including for example FIG. 8A and/or FIG. 8B and/or FIG. 8C and/or FIG. 8D. In some embodiments, TILs that exhibit greater than 180000 pg/mL Granzyme B secretion are TILs produced by the expansion methods of the present invention, including for example FIG. 8A and/or FIG. 8B and/or FIG. 8C and/or FIG. 8D. In some embodiments, TILs that exhibit greater than 200000 pg/mL Granzyme B secretion are TILs produced by the expansion methods of the present invention, including for example FIG. 8A and/or FIG. 8B and/or FIG. 8C and/or FIG. 8D. In some embodiments, TILs that exhibit greater than 220000 pg/mL Granzyme B secretion are TILs produced by the expansion methods of the present invention, including for example FIG. 8A and/or FIG. 8B and/or FIG. 8C and/or FIG. 8D. In some embodiments, TILs that exhibit greater than 240000 pg/mL Granzyme B secretion are TILs produced by the expansion methods of the present invention, including for example FIG. 8A and/or FIG. 8B and/or FIG. 8C and/or FIG. 8D. In some embodiments, TILs that exhibit greater than 260000 pg/mL Granzyme B secretion are TILs produced by the expansion methods of the present invention, including for example FIG. 8A and/or FIG. 8B and/or FIG. 8C and/or FIG. 8D. In some embodiments, TILs that exhibit greater than 280000 pg/mL Granzyme B secretion are TILs produced by the expansion methods of the present invention, including for example FIG. 8A and/or FIG. 8B and/or FIG. 8C and/or FIG. 8D. In some embodiments, TILs that exhibit greater than 300000 pg/mL Granzyme B secretion are TILs produced by the expansion methods of the present invention, including for example FIG. 8A and/or FIG. 8B and/or FIG. 8C and/or FIG. 8D.

In some embodiments, the expansion methods of the present invention produce an expanded population of TILs that exhibits increased Granzyme B secretion in vitro including for example TILs as provided in FIG. 8A and/or FIG. 8B and/or FIG. 8C and/or FIG. 8D, as compared to non-expanded population of TILs. In some embodiments, Granzyme B secretion of the expanded population of TILs of the present invention is increased by at least one-fold to fifty-fold or more as compared to non-expanded population of TILs. In some embodiments, IFN-γ secretion is increased by at least one-fold, at least two-fold, at least three-fold, at least four-fold, at least five-fold, at least six-fold, at least seven-fold, at least eight-fold, at least nine-fold, at least ten-fold, at least twenty-fold, at least thirty-fold, at least forty-fold, at least fifty-fold or more as compared to non-expanded population of TILs. In some embodiments, Granzyme B secretion of the expanded population of TILs of the present invention is increased by at least one-fold as compared to non-expanded population of TILs. In some embodiments, Granzyme B secretion of the expanded population of TILs of the present invention is increased by at least two-fold as compared to non-expanded population of TILs. In some embodiments, Granzyme B secretion of the expanded population of TILs of the present invention is increased by at least three-fold as compared to non-expanded population of TILs. In some embodiments, Granzyme B secretion of the expanded population of TILs of the present invention is increased by at least four-fold as compared to non-expanded population of TILs. In some embodiments, Granzyme B secretion of the expanded population of TILs of the present invention is increased by at least five-fold as compared to non-expanded population of TILs. In some embodiments, Granzyme B secretion of the expanded population of TILs of the present invention is increased by at least six-fold as compared to non-expanded population of TILs. In some embodiments, Granzyme B secretion of the expanded population of TILs of the present invention is increased by at least seven-fold as compared to non-expanded population of TILs. In some embodiments, Granzyme B secretion of the expanded population of TILs of the present invention is increased by at least eight-fold as compared to non-expanded population of TILs. In some embodiments, Granzyme B secretion of the expanded population of TILs of the present invention is increased by at least nine-fold as compared to non-expanded population of TILs. In some embodiments, Granzyme B secretion of the expanded population of TILs of the present invention is increased by at least ten-fold as compared to non-expanded population of TILs. In some embodiments, Granzyme B secretion of the expanded population of TILs of the present invention is increased by at least twenty-fold as compared to non-expanded population of TILs. In some embodiments, Granzyme B secretion of the expanded population of TILs of the present invention is increased by at least thirty-fold as compared to non-expanded population of TILs. In some embodiments, Granzyme B secretion of the expanded population of TILs of the present invention is increased by at least forty-fold as compared to non-expanded population of TILs. In some embodiments, Granzyme B secretion of the expanded population of TILs of the present invention is increased by at least fifty-fold as compared to non-expanded population of TILs.

In some embodiments, TILs capable of at least one-fold, two-fold, three-fold, four-fold, or five-fold or more lower levels of TNF-α (i.e., TNF-alpha) secretion as compared to IFN-γ secretion are TILs produced by the expansion methods of the present invention, including, for example FIG. 8A and/or FIG. 8B and/or FIG. 8C and/or FIG. 8D methods. In some embodiments, TILs capable of at least one-fold lower levels of TNF-α secretion as compared to IFN-γ secretion are TILs produced by the expansion methods of the present invention, including, for example FIG. 8A and/or FIG. 8B and/or FIG. 8C and/or FIG. 8D methods. In some embodiments, TILs capable of at least two-fold lower levels of TNF-α secretion as compared to IFN-γ secretion are TILs produced by the expansion methods of the present invention, including, for example FIG. 8A and/or FIG. 8B and/or FIG. 8C and/or FIG. 8D methods. In some embodiments, TILs capable of at least three-fold lower levels of TNF-α secretion as compared to IFN-γ secretion are TILs produced by the expansion methods of the present invention, including, for example FIG. 8A and/or FIG. 8B and/or FIG. 8C and/or FIG. 8D methods. In some embodiments, TILs capable of at least four-fold lower levels of TNF-α secretion as compared to IFN-γ secretion are TILs produced by the expansion methods of the present invention, including, for example FIG. 8A and/or FIG. 8B and/or FIG. 8C and/or FIG. 8D methods. In some embodiments, TILs capable of at least five-fold lower levels of TNF-α secretion as compared to IFN-γ secretion are TILs produced by the expansion methods of the present invention, including, for example FIG. 8A and/or FIG. 8B and/or FIG. 8C and/or FIG. 8D methods.

In some embodiments, TILs capable of at least 200 pg/mL/5e5 cells to about 10,000 pg/mL/5e5 cells or more TNF-α (i.e., TNF-alpha) secretion are TILs produced by the expansion methods of the present invention, including, for example FIG. 8A and/or FIG. 8B and/or FIG. 8C and/or FIG. 8D methods. In some embodiments, TILs capable of at least 500 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 1000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 2000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 3000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 4000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 5000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 6000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 7000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 8000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods. In some embodiments, TILs capable of at least 9000 pg/mL/5e5 cells to about 10,000 pg/mL/5e5 cells or more TNF-α secretion are TILs produced by the expansion methods of the present invention, including, for example FIG. 8A and/or FIG. 8B and/or FIG. 8C and/or FIG. 8D methods.

In some embodiments, IFN-γ and granzyme B levels are measured to determine the phenotypic characteristics of the TILs produced by the expansion methods of the present invention, including, for example FIG. 8A and/or FIG. 8B and/or FIG. 8C and/or FIG. 8D methods. In some embodiments, IFN-γ and TNF-α levels are measured to determine the phenotypic characteristics of the TILs produced by the expansion methods of the present invention, including, for example FIG. 8A and/or FIG. 8B and/or FIG. 8C and/or FIG. 8D methods. In some embodiments, granzyme B and TNF-α levels are measured to determine the phenotypic characteristics of the TILs produced by the expansion methods of the present invention, including, for example FIG. 8A and/or FIG. 8B and/or FIG. 8C and/or FIG. 8D methods. In some embodiments, IFN-γ, granzyme B and TNF-α levels are measured to determine the phenotypic characteristics of the TILs produced by the expansion methods of the present invention, including, for example FIG. 8A and/or FIG. 8B and/or FIG. 8C and/or FIG. 8D methods.

In some embodiments, the phenotypic characterization is examined after cryopreservation.

### G. Additional Process Embodiments

In some embodiments, the invention provides a method for expanding tumor infiltrating lymphocytes (TILs) into a therapeutic population of TILs comprising: (a) obtaining a first population of TILs from a tumor resected from a subject by processing a tumor sample obtained from the subject into multiple tumor fragments; (b) performing a priming first expansion by culturing the first population of TILs in a cell culture medium comprising IL-2 and OKT-3, wherein the priming first expansion is performed for about 1 to 7 days or about about 1 to 8 days to obtain the second population of TILs, wherein the second population of TILs is greater in number than the first population of TILs; (c) performing a rapid second expansion by contacting the second population of TILs with a cell culture medium comprising IL-2, OKT-3 and exogenous antigen presenting cells (APCs) to produce a third population of TILs, wherein the rapid second expansion is performed for about 1 to 11 days or about 1 to 10 days to obtain the third population of TILs, wherein the third population of TILs is a therapeutic population of TILs; (d) harvesting the therapeutic population of TILs obtained from step (c), and (e) at any time prior to or after step (d), gene-editing at least a portion of the TIL cells to express at least one immunomodulatory composition on the surface of the TIL cells. In some embodiments, the step of rapid second expansion is split into a plurality of steps to achieve a scaling up of the culture by: (1) performing the rapid second expansion by culturing the second population of TILs in a small scale culture in a first container, e.g., a G-REX-100MCS container, for a period of about 3 to 4 days, or about 2 to 4 days, and then (2) effecting the transfer of the second population of TILs from the small scale culture to a second container larger than the first container, e.g., a G-REX-500MCS container, wherein in the second container the second population of TILs from the small scale culture is cultured in a larger scale culture for a period of about 4 to 7 days, or about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX-100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days, or about about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX-100MCS container, for a period of about 3 to 4 days, or about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX-500MCS containers, wherein in each second container the portion of the second population of TILs transferred from the small scale culture to such second container is cultured in a larger scale culture for a period of about 4 to 7 days, or about 4 to 8 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX-100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX-500MCS containers, wherein in each second container the portion of the second population of TILs transferred from the small scale culture to such second container is cultured in a larger scale culture for a period of about 5 to 7 days. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

In some embodiments, the invention provides a method for expanding tumor infiltrating lymphocytes (TILs) into a therapeutic population of TILs comprising: (a) obtaining a first population of TILs from a tumor resected from a subject by processing a tumor sample obtained from the subject into multiple tumor fragments; (b) performing a priming first expansion by culturing the first population of TILs in a cell culture medium comprising IL-2 and OKT-3, wherein the priming first expansion is performed for about 1 to 8 days to obtain the second population of TILs, wherein the second population of TILs is greater in number than the first population of TILs; (c) performing a rapid second expansion by contacting the second population of TILs with a cell culture medium comprising IL-2, OKT-3 and exogenous antigen presenting cells (APCs) to produce a third population of TILs, wherein the rapid second expansion is performed for about 1 to 8 days to obtain the third population of TILs, wherein the third population of TILs is a therapeutic population of TILs; (d) harvesting the therapeutic population of TILs obtained from step (c); and (e) at any time prior to or after step (d), gene-editing at least a portion of the TIL cells to express at least one immunomodulatory composition on the surface of the TIL cells). In some embodiments, the step of rapid second expansion is split into a plurality of steps to achieve a scaling up of the culture by: (1) performing the rapid second expansion by culturing the second population of TILs in a small scale culture in a first container, e.g., a G-REX-100MCS container, for a period of about 2 to 4 days, and then (2) effecting the transfer of the second population of TILs from the small scale culture to a second container larger than the first container, e.g., a G-REX-500MCS container, wherein in the second container the second population of TILs from the small scale culture is cultured in a larger scale culture for a period of about 4 to 8 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX-100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX-100MCS container, for a period of about 2 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX-500MCS containers, wherein in each second container the portion of the second population of TILs transferred from the small scale culture to such second container is cultured in a larger scale culture for a period of about 4 to 6 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX-100MCS container, for a period of about 3 to 4 days, and then (2) effecting the transfer and apportioning of the second population of TILs from the first small scale culture into and amongst 2, 3 or 4 second containers that are larger in size than the first container, e.g., G-REX-500MCS containers, wherein in each second container the portion of the second population of TILs transferred from the small scale culture to such second container is cultured in a larger scale culture for a period of about 4 to 5 days. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

In some embodiments, the invention provides a method for expanding tumor infiltrating lymphocytes (TILs) into a therapeutic population of TILs comprising: (a) obtaining a first population of TILs from a tumor resected from a subject by processing a tumor sample obtained from the subject into multiple tumor fragments; (b) performing a priming first expansion by culturing the first population of TILs in a cell culture medium comprising IL-2 and OKT-3, wherein the priming first expansion is performed for about 1 to 7 days to obtain the second population of TILs, wherein the second population of TILs is greater in number than the first population of TILs; (c) performing a rapid second expansion by contacting the second population of TILs with a cell culture medium comprising IL-2, OKT-3 and exogenous antigen presenting cells (APCs) to produce a third population of TILs, wherein the rapid second expansion is performed for about 1 to 11 days to obtain the third population of TILs, wherein the third population of TILs is a therapeutic population of TILs; and (d) harvesting the therapeutic population of TILs obtained from step (c); and (e) at any time prior to or after step (d), gene-editing at least a portion of the TIL cells to express at least one immunomodulatory composition on the surface of the TIL cells. In some embodiments, the step of rapid second expansion is split into a plurality of steps to achieve a scaling up of the culture by: (1) performing the rapid second expansion by culturing the second population of TILs in a small scale culture in a first container, e.g., a G-REX-100MCS container, for a period of about 3 to 4 days, and then (2) effecting the transfer of the second population of TILs from the small scale culture to a second container larger than the first container, e.g., a G-REX-500MCS container, wherein in the second container the second population of TILs from the small scale culture is cultured in a larger scale culture for a period of about 4 to 7 days. In some embodiments, the step of rapid expansion is split into a plurality of steps to achieve a scaling out of the culture by: (1) performing the rapid second expansion by culturing the second population of TILs in a first small scale culture in a first container, e.g., a G-REX-100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the second population of TILs from the first small scale culture transferred to such second container is cultured in a second small scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX-100MCS container, for a period of about 3 to 4 days, and then (2) effecting the transfer and apportioning of the second population of TILs from the first small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX-500MCS containers, wherein in each second container the portion of the second population of TILs transferred from the small scale culture to such second container is cultured in a larger scale culture for a period of about 4 to 7 days. In some embodiments, the step of rapid expansion is split into a plurality of steps to achieve a scaling out and scaling up of the culture by: (1) performing the rapid second expansion by culturing the second population of TILs in a small scale culture in a first container, e.g., a G-REX-100MCS container, for a period of about 4 days, and then (2) effecting the transfer and apportioning of the second population of TILs from the first small scale culture into and amongst 2, 3 or 4 second containers that are larger in size than the first container, e.g., G-REX-g500MCS containers, wherein in each second container the portion of the second population of TILs transferred from the small scale culture to such second container is cultured in a larger scale culture for a period of about 5 days. In some embodiments, the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

In other embodiments, the invention provides the method described in any of the preceding paragraphs as applicable above modified such that in step (b) the primary first expansion is performed by contacting the first population of TILs with a culture medium which further comprises exogenous antigen-presenting cells (APCs), wherein the number of APCs in the culture medium in step (c) is greater than the number of APCs in the culture medium in step (b).

In other embodiments, the invention provides the method described in any of the preceding paragraphs as applicable above modified such that in step (c) the culture medium is supplemented with additional exogenous APCs.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 20:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 10:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 9:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 8:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 7:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 6:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 5:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 4:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 3:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.9:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.8:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.7:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.6:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.5:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.4:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.3:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.2:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2.1:1.

In other embodiments, the invention provides the method described in any of the preceding paragraphs as applicable above modified such that the ratio of number of APCs added in the rapid second expansion to the number of APCs added in step (b) is selected from a range of from at or about 1.1:1 to at or about 2:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 10:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 5:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 4:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 3:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.9:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.8:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.7:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.6:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.5:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.4:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.3:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.2:1.

In other embodiments, the invention provides the method described in any of the preceding paragraphs as applicable above modified such that the ratio of number of APCs added in the rapid second expansion to the number of APCs added in step (b) is selected from a range of from at or about 2:1 to at or about 2.1:1.

In other embodiments, the invention provides the method described in any of the preceding paragraphs as applicable above modified such that the ratio of number of APCs added in the rapid second expansion to the number of APCs added in step (b) is at or about 2:1.

In other embodiments, the invention provides the method described in any of the preceding paragraphs as applicable above modified such that the ratio of number of APCs added in the rapid second expansion to the number of APCs added in step (b) is at or about 1.1:1, 1.2:1, 1.3:1, 1.4:1, 1.5:1, 1.6:1, 1.7:1, 1.8:1, 1.9:1, 2:1, 2.1:1, 2.2:1, 2.3:1, 2.4:1, 2.5:1, 2.6:1, 2.7:1, 2.8:1, 2.9:1, 3:1, 3.1:1, 3.2:1, 3.3:1, 3.4:1, 3.5:1, 3.6:1, 3.7:1, 3.8:1, 3.9:1, 4:1, 4.1:1, 4.2:1, 4.3:1, 4.4:1, 4.5:1, 4.6:1, 4.7:1, 4.8:1, 4.9:1, or 5:1.

In other embodiments, the invention provides the method described in any of the preceding paragraphs as applicable above modified such that the number of APCs added in the primary first expansion is at or about 1×10⁸, 1.1×10⁸, 1.2×10⁸, 1.3×10⁸, 1.4×10⁸, 1.5×10⁸, 1.6×10⁸, 1.7×10⁸, 1.8×10⁸, 1.9×10⁸, 2×10⁸, 2.1×10⁸, 2.2×10⁸, 2.3×10⁸, 2.4×10⁸, 2.5×10⁸, 2.6×10⁸, 2.7×10⁸, 2.8×10⁸, 2.9×10⁸, 3×10⁸, 3.1×10⁸, 3.2×10⁸, 3.3×10⁸, 3.4×10⁸ or 3.5×10⁸ APCs, and such that the number of APCs added in the rapid second expansion is at or about 3.5×10⁸, 3.6×10⁸, 3.7×10⁸, 3.8×10⁸, 3.9×10⁸, 4×10⁸, 4.1×10⁸, 4.2×10⁸, 4.3×10⁸, 4.4×10⁸, 4.5×10⁸, 4.6×10⁸, 4.7×10⁸, 4.8×10⁸, 4.9×10⁸, 5×10⁸, 5.1×10⁸, 5.2×10⁸, 5.3×10⁸, 5.4×10⁸, 5.5×10⁸, 5.6×10⁸, 5.7×10⁸, 5.8×10⁸, 5.9×10⁸, 6×10⁸, 6.1×10⁸, 6.2×10⁸, 6.3×10⁸, 6.4×10⁸, 6.5×10⁸, 6.6×10⁸, 6.7×10⁸, 6.8×10⁸, 6.9×10⁸, 7×10⁸, 7.1×10⁸, 7.2×10⁸, 7.3×10⁸, 7.4×10⁸, 7.5×10⁸, 7.6×10⁸, 7.7×10⁸, 7.8×10⁸, 7.9×10⁸, 8×10⁸, 8.1×10⁸, 8.2×10⁸, 8.3×10⁸, 8.4×10⁸, 8.5×10⁸, 8.6×10⁸, 8.7×10⁸, 8.8×10⁸, 8.9×10⁸, 9×10⁸, 9.1×10⁸, 9.2×10⁸, 9.3×10⁸, 9.4×10⁸, 9.5×10⁸, 9.6×10⁸, 9.7×10⁸, 9.8×10⁸, 9.9×10⁸ or 1×10⁹ APCs.

In other embodiments, the invention provides the method described in any of the preceding paragraphs as applicable above modified such that the number of APCs added in the primary first expansion is selected from the range of at or about 1×10⁸ APCs to at or about 3.5×10⁸ APCs, and wherein the number of APCs added in the rapid second expansion is selected from the range of at or about 3.5×10⁸ APCs to at or about 1×10⁹ APCs.

In other embodiments, the invention provides the method described in any of the preceding paragraphs as applicable above modified such that the number of APCs added in the primary first expansion is selected from the range of at or about 1.5×10⁸ APCs to at or about 3×10⁸ APCs, and wherein the number of APCs added in the rapid second expansion is selected from the range of at or about 4×10⁸ APCs to at or about 7.5×10⁸ APCs.

In other embodiments, the invention provides the method described in any of the preceding paragraphs as applicable above modified such that the number of APCs added in the primary first expansion is selected from the range of at or about 2×10⁸ APCs to at or about 2.5×10⁸ APCs, and wherein the number of APCs added in the rapid second expansion is selected from the range of at or about 4.5×10⁸ APCs to at or about 5.5×10⁸ APCs.

In other embodiments, the invention provides the method described in any of the preceding paragraphs as applicable above modified such that at or about 2.5×10⁸ APCs are added to the primary first expansion and at or about 5×10⁸ APCs are added to the rapid second expansion.

In other embodiments, the invention provides the method described in any of the preceding paragraphs as applicable above modified such that the antigen-presenting cells are peripheral blood mononuclear cells (PBMCs).

In other embodiments, the invention provides the method described in any of the preceding paragraphs as applicable above modified such that the multiple tumor fragments are distributed into a plurality of separate containers, in each of which separate containers the first population of TILs is obtained in step (a), the second population of TILs is obtained in step (b), and the third population of TILs is obtained in step (c), and the therapeutic populations of TILs from the plurality of containers in step (c) are combined to yield the harvested TIL population from step (d).

In other embodiments, the invention provides the method described in any of the preceding paragraphs as applicable above modified such that the multiple tumors are evenly distributed into the plurality of separate containers.

In other embodiments, the invention provides the method described in any of the preceding paragraphs as applicable above modified such that the plurality of separate containers comprises at least two separate containers.

In other embodiments, the invention provides the method described in any of the preceding paragraphs as applicable above modified such that the plurality of separate containers comprises from two to twenty separate containers.

In other embodiments, the invention provides the method described in any of the preceding paragraphs as applicable above modified such that the plurality of separate containers comprises from two to fifteen separate containers.

In other embodiments, the invention provides the method described in any of the preceding paragraphs as applicable above modified such that the plurality of separate containers comprises from two to ten separate containers.

In other embodiments, the invention provides the method described in any of the preceding paragraphs as applicable above modified such that the plurality of separate containers comprises from two to five separate containers.

In other embodiments, the invention provides the method described in any of the preceding paragraphs as applicable above modified such that the plurality of separate containers comprises 2, 3, 4, 5, 6, 7, 8, 9, 10, 11, 12, 13, 14, 15, 16, 17, 18, 19, or 20 separate containers.

In other embodiments, the invention provides the method described in any of the preceding paragraphs as applicable above modified such that for each container in which the priming first expansion is performed on a first population of TILs in step (b) the rapid second expansion in step (c) is performed in the same container on the second population of TILs produced from such first population of TILs.

In other embodiments, the invention provides the method described in any of the preceding paragraphs as applicable above modified such that each of the separate containers comprises a first gas-permeable surface area.

In other embodiments, the invention provides the method described in any of the preceding paragraphs as applicable above modified such that the multiple tumor fragments are distributed in a single container.

In other embodiments, the invention provides the method described in any of the preceding paragraphs as applicable above modified such that the single container comprises a first gas-permeable surface area.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell layer to at or about three cell layers.

In other embodiments, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In other embodiments, the invention provides the method described in any of the preceding paragraphs as applicable above modified such that in step (b) the APCs are layered onto the first gas-permeable surface area at an average thickness of at or about 2 cell layers.

In other embodiments, the invention provides the method described in any of the preceding paragraphs as applicable above modified such that in step (b) the APCs are layered onto the first gas-permeable surface area at an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers.

In other embodiments, the invention provides the method described in any of the preceding paragraphs as applicable above modified such that in step (c) the APCs are layered onto the first gas-permeable surface area at an average thickness of at or about 3 cell layers to at or about 10 cell layers.

In other embodiments, the invention provides the method described in any of the preceding paragraphs as applicable above modified such that in step (c) the APCs are layered onto the first gas-permeable surface area at an average thickness of at or about 4 cell layers to at or about 8 cell layers.

In other embodiments, the invention provides the method described in any of the preceding paragraphs as applicable above modified such that in step (c) the APCs are layered onto the first gas-permeable surface area at an average thickness of at or about 3, 4, 5, 6, 7, 8, 9 or 10 cell layers.

In other embodiments, the invention provides the method described in any of the preceding paragraphs as applicable above modified such that in step (c) the APCs are layered onto the first gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In other embodiments, the invention provides the method described in any of the preceding paragraphs as applicable above modified such that in step (b) the priming first expansion is performed in a first container comprising a first gas-permeable surface area and in step (c) the rapid second expansion is performed in a second container comprising a second gas-permeable surface area.

In other embodiments, the invention provides the method described in any of the preceding paragraphs as applicable above modified such that the second container is larger than the first container.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell layer to at or about three cell layers.

In other embodiments, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In other embodiments, the invention provides the method described in any of the preceding paragraphs as applicable above modified such that in step (b) the APCs are layered onto the first gas-permeable surface area at an average thickness of at or about 2 cell layers.

In other embodiments, the invention provides the method described in any of the preceding paragraphs as applicable modified such that in step (b) the APCs are layered onto the first gas-permeable surface area at an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers.

In other embodiments, the invention provides the method described in any of the preceding paragraphs as applicable above modified such that in step (c) the APCs are layered onto the second gas-permeable surface area at an average thickness of at or about 3 cell layers to at or about 10 cell layers.

In other embodiments, the invention provides the method described in any of the preceding paragraphs as applicable above modified such that in step (c) the APCs are layered onto the second gas-permeable surface area at an average thickness of at or about 4 cell layers to at or about 8 cell layers.

In other embodiments, the invention provides the method described in any of the preceding paragraphs as applicable above modified such that in step (c) the APCs are layered onto the second gas-permeable surface area at an average thickness of at or about 3, 4, 5, 6, 7, 8, 9 or 10 cell layers.

In other embodiments, the invention provides the method described in any of the preceding paragraphs as applicable modified such that in step (c) the APCs are layered onto the second gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In other embodiments, the invention provides the method described in any of the preceding paragraphs as applicable above modified such that in step (b) the priming first expansion is performed in a first container comprising a first gas-permeable surface area and in step (c) the rapid second expansion is performed in the first container.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein in step (b) the APCs are layered onto the first gas-permeable surface area at an average thickness of at or about one cell layer to at or about three cell layers.

In other embodiments, the invention provides the method described in any of the preceding paragraphs as applicable above modified such that in step (b) the APCs are layered onto the first gas-permeable surface area at an average thickness of at or about 1.5 cell layers to at or about 2.5 cell layers.

In other embodiments, the invention provides the method described in any of the preceding paragraphs as applicable above modified such that in step (b) the APCs are layered onto the first gas-permeable surface area at an average thickness of at or about 2 cell layers.

In other embodiments, the invention provides the method described any of the preceding paragraphs as applicable above modified such that in step (b) the APCs are layered onto the first gas-permeable surface area at an average thickness of at or about 1, 1.1, 1.2, 1.3, 1.4, 1.5, 1.6, 1.7, 1.8, 1.9, 2, 2.1, 2.2, 2.3, 2.4, 2.5, 2.6, 2.7, 2.8, 2.9 or 3 cell layers.

In other embodiments, the invention provides the method described in any of the preceding paragraphs as applicable above modified such that in step (c) the APCs are layered onto the first gas-permeable surface area at an average thickness of at or about 3 cell layers to at or about 10 cell layers.

In other embodiments, the invention provides the method described in any of the preceding paragraphs as applicable above modified such that in step (c) the APCs are layered onto the first gas-permeable surface area at an average thickness of at or about 4 cell layers to at or about 8 cell layers.

In other embodiments, the invention provides the method described in any of the preceding paragraphs as applicable above modified such that in step (c) the APCs are layered onto the first gas-permeable surface area at an average thickness of at or about 3, 4, 5, 6, 7, 8, 9 or 10 cell layers.

In other embodiments, the invention provides the method described in any of the preceding paragraphs as applicable above modified such that in step (c) the APCs are layered onto the first gas-permeable surface area at an average thickness of at or about 4, 4.1, 4.2, 4.3, 4.4, 4.5, 4.6, 4.7, 4.8, 4.9, 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, 7.5, 7.6, 7.7, 7.8, 7.9 or 8 cell layers.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:10.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:9.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:8.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:7.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:6.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:5.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:4.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:3.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.1 to at or about 1:2.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.2 to at or about 1:8.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.3 to at or about 1:7.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.4 to at or about 1:6.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.5 to at or about 1:5.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.6 to at or about 1:4.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.7 to at or about 1:3.5.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.8 to at or about 1:3.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:1.9 to at or about 1:2.5.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from the range of at or about 1:2.

In other embodiments, the invention provides the method described in any of the preceding paragraphs as applicable above modified such that in step (b) the primary first expansion is performed by supplementing the cell culture medium of the first population of TILs with additional antigen-presenting cells (APCs), wherein the number of APCs added in step (c) is greater than the number of APCs added in step (b), and wherein the ratio of the average number of layers of APCs layered in step (b) to the average number of layers of APCs layered in step (c) is selected from at or about 1:1.1, 1:1.2, 1:1.3, 1:1.4, 1:1.5, 1:1.6, 1:1.7, 1:1.8, 1:1.9, 1:2, 1:2.1, 1:2.2, 1:2.3, 1:2.4, 1:2.5, 1:2.6, 1:2.7, 1:2.8, 1:2.9, 1:3, 1:3.1, 1:3.2, 1:3.3, 1:3.4, 1:3.5, 1:3.6, 1:3.7, 1:3.8, 1:3.9, 1:4, 1:4.1, 1:4.2, 1:4.3, 1:4.4, 1:4.5, 1:4.6, 1:4.7, 1:4.8, 1:4.9, 1:5, 1:5.1, 1:5.2, 1:5.3, 1:5.4, 1:5.5, 1:5.6, 1:5.7, 1:5.8, 1:5.9, 1:6, 1:6.1, 1:6.2, 1:6.3, 1:6.4, 1:6.5, 1:6.6, 1:6.7, 1:6.8, 1:6.9, 1:7, 1:7.1, 1:7.2, 1:7.3, 1:7.4, 1:7.5, 1:7.6, 1:7.7, 1:7.8, 1:7.9, 1:8, 1:8.1, 1:8.2, 1:8.3, 1:8.4, 1:8.5, 1:8.6, 1:8.7, 1:8.8, 1:8.9, 1:9, 1:9.1, 1:9.2, 1:9.3, 1:9.4, 1:9.5, 1:9.6, 1:9.7, 1:9.8, 1:9.9 or 1:10.

In other embodiments, the invention provides the method described in any of the preceding paragraphs as applicable above modified such that the ratio of the number of TILs in the second population of TILs to the number of TILs in the first population of TILs is at or about 1.5:1 to at or about 100:1.

In other embodiments, the invention provides the method described in any of the preceding paragraphs as applicable above modified such that the ratio of the number of TILs in the second population of TILs to the number of TILs in the first population of TILs is at or about 50:1.

In other embodiments, the invention provides the method described in any of the preceding paragraphs as applicable above modified such that the ratio of the number of TILs in the second population of TILs to the number of TILs in the first population of TILs is at or about 25:1.

In other embodiments, the invention provides the method described in any of the preceding paragraphs as applicable above modified such that the ratio of the number of TILs in the second population of TILs to the number of TILs in the first population of TILs is at or about 20:1.

In other embodiments, the invention provides the method described in any of the preceding paragraphs as applicable above modified such that the ratio of the number of TILs in the second population of TILs to the number of TILs in the first population of TILs is at or about 10:1.

In other embodiments, the invention provides the method described in any of the preceding paragraphs as applicable above modified such that the second population of TILs is at least at or about 50-fold greater in number than the first population of TILs.

In other embodiments, the invention provides the method described in any of the preceding paragraphs as applicable above modified such that the second population of TILs is at least at or about 1-, 2-, 3-, 4-, 5-, 6-, 7-, 8-, 9-, 10-, 11-, 12-, 13-, 14-, 15-, 16-, 17-, 18-, 19-, 20-, 21-, 22-, 23-, 24-, 25-, 26-, 27-, 28-, 29-, 30-, 31-, 32-, 33-, 34-, 35-, 36-, 37-, 38-, 39-, 40-, 41-, 42-, 43-, 44-, 45-, 46-, 47-, 48-, 49- or 50-fold greater in number than the first population of TILs.

In other embodiments, the invention provides the method described in any of the preceding paragraphs as applicable above modified such that at or about 2 days or at or about 3 days after the commencement of the second period in step (c), the cell culture medium is supplemented with additional IL-2.

In other embodiments, the invention provides the method described in any of the preceding paragraphs as applicable above modified to further comprise the step of cryopreserving the harvested TIL population in step (d) using a cryopreservation process.

In other embodiments, the invention provides the method described in any of the preceding paragraphs as applicable above modified to comprise performing after step (d) the additional step of (e) transferring the harvested TIL population from step (d) to an infusion bag that optionally contains HypoThermosol.

In other embodiments, the invention provides the method described in any of the preceding paragraphs as applicable above modified to comprise the step of cryopreserving the infusion bag comprising the harvested TIL population in step (e) using a cryopreservation process.

In other embodiments, the invention provides the method described in any of the preceding paragraphs as applicable above modified such that the cryopreservation process is performed using a 1:1 ratio of harvested TIL population to cryopreservation media.

In other embodiments, the invention provides the method described in any of the preceding paragraphs as applicable above modified such that the antigen-presenting cells are peripheral blood mononuclear cells (PBMCs).

In other embodiments, the invention provides the method described in any of the preceding paragraphs as applicable above modified such that the PBMCs are irradiated and allogeneic.

In other embodiments, the invention provides the method described in any of the preceding paragraphs as applicable above modified such that the total number of APCs added to the cell culture in step (b) is 2.5×10⁸.

In other embodiments, the invention provides the method described in any of the preceding paragraphs as applicable above modified such that the total number of APCs added to the cell culture in step (c) is 5×10⁸.

In other embodiments, the invention provides the method described in any of the preceding paragraphs as applicable above modified such that the APCs are PBMCs.

In other embodiments, the invention provides the method described in any of the preceding paragraphs as applicable above modified such that the PBMCs are irradiated and allogeneic.

In other embodiments, the invention provides the method described in any of the preceding paragraphs as applicable above modified such that the antigen-presenting cells are artificial antigen-presenting cells.

In other embodiments, the invention provides the method described in any of the preceding paragraphs as applicable above modified such that the harvesting in step (d) is performed using a membrane-based cell processing system.

In other embodiments, the invention provides the method described in any of the preceding paragraphs as applicable above modified such that the harvesting in step (d) is performed using a LOVO cell processing system.

In other embodiments, the invention provides the method described in any of the preceding paragraphs as applicable above modified such that the multiple fragments comprise at or about 5 to at or about 60 fragments per container in step (b).

In other embodiments, the invention provides the method described in any of the preceding paragraphs as applicable above modified such that the multiple fragments comprise at or about 10 to at or about 60 fragments per container in step (b).

In other embodiments, the invention provides the method described in any of the preceding paragraphs as applicable above modified such that the multiple fragments comprise at or about 15 to at or about 60 fragments per container in step (b).

In other embodiments, the invention provides the method described in any of the preceding paragraphs as applicable above modified such that the multiple fragments comprise at or about 20 to at or about 60 fragments per container in step (b).

In other embodiments, the invention provides the method described in any of the preceding paragraphs as applicable above modified such that the multiple fragments comprise at or about 25 to at or about 60 fragments per container in step (b).

In other embodiments, the invention provides the method described in any of the preceding paragraphs as applicable above modified such that the multiple fragments comprise at or about 30 to at or about 60 fragments per container in step (b).

In other embodiments, the invention provides the method described in any of the preceding paragraphs as applicable above modified such that the multiple fragments comprise at or about 35 to at or about 60 fragments per container in step (b).

In other embodiments, the invention provides the method described in any of the preceding paragraphs as applicable above modified such that the multiple fragments comprise at or about 40 to at or about 60 fragments per container in step (b).

In other embodiments, the invention provides the method described in any of the preceding paragraphs as applicable above modified such that the multiple fragments comprise at or about 45 to at or about 60 fragments per container in step (b).

In other embodiments, the invention provides the method described in any of the preceding paragraphs as applicable above modified such that the multiple fragments comprise at or about 50 to at or about 60 fragments per container in step (b).

In other embodiments, the invention provides the method described in any of the preceding paragraphs as applicable above modified such that the multiple fragments comprise at or about 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 or 60 fragment(s) per container in step (b).

In other embodiments, the invention provides the method described in any of the preceding paragraphs as applicable above modified such that each fragment has a volume of at or about 27 mm³.

In other embodiments, the invention provides the method described in any of the preceding paragraphs as applicable above modified such that each fragment has a volume of at or about 20 mm³ to at or about 50 mm³.

In other embodiments, the invention provides the method described in any of the preceding paragraphs as applicable above modified such that each fragment has a volume of at or about 21 mm³ to at or about 30 mm³.

In other embodiments, the invention provides the method described in any of the preceding paragraphs as applicable above modified such that each fragment has a volume of at or about 22 mm³ to at or about 29.5 mm³.

In other embodiments, the invention provides the method described in any of the preceding paragraphs as applicable above modified such that each fragment has a volume of at or about 23 mm³ to at or about 29 mm³.

In other embodiments, the invention provides the method described in any of the preceding paragraphs as applicable above modified such that each fragment has a volume of at or about 24 mm³ to at or about 28.5 mm³.

In other embodiments, the invention provides the method described in any of the preceding paragraphs as applicable above modified such that each fragment has a volume of at or about 25 mm³ to at or about 28 mm³.

In other embodiments, the invention provides the method described in any of the preceding paragraphs as applicable above modified such that each fragment has a volume of at or about 26.5 mm³ to at or about 27.5 mm³.

In other embodiments, the invention provides the method described in any of the preceding paragraphs as applicable above modified such that each fragment has a volume of at or about 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 mm³.

In other embodiments, the invention provides the method described in any of the preceding paragraphs as applicable above modified such that the multiple fragments comprise at or about 30 to at or about 60 fragments with a total volume of at or about 1300 mm³ to at or about 1500 mm³.

In other embodiments, the invention provides the method described in any of the preceding paragraphs as applicable above modified such that the multiple fragments comprise at or about 50 fragments with a total volume of at or about 1350 mm³.

In other embodiments, the invention provides the method described in any of the preceding paragraphs as applicable above modified such that the multiple fragments comprise at or about 50 fragments with a total mass of at or about 1 gram to at or about 1.5 grams.

In other embodiments, the invention provides the method described in any of the preceding paragraphs as applicable above modified such that the cell culture medium is provided in a container that is a G-container or a Xuri cellbag.

In other embodiments, the invention provides the method described in any of the preceding paragraphs as applicable above modified such that the IL-2 concentration in the cell culture medium is about 10,000 IU/mL to about 5,000 IU/mL.

In other embodiments, the invention provides the method described in any of the preceding paragraphs as applicable above modified such that the IL-2 concentration in the cell culture medium is about 6,000 IU/mL.

In other embodiments, the invention provides the method described in any of the preceding paragraphs as applicable above modified such that the cryopreservation media comprises dimethlysulfoxide (DMSO).

In other embodiments, the invention provides the method described in any of the preceding paragraphs as applicable above modified such that the cryopreservation media comprises 7% to 10% DMSO.

In other embodiments, the invention provides the method described in any of the preceding paragraphs as applicable above modified such that the first period in step (b) is performed within a period of at or about 1 day, 2 days, 3 days, 4 days, 5 days, 6 days, or 7 days.

In other embodiments, the invention provides the method described in any of the preceding paragraphs as applicable above modified such that the second period in step (c) is performed within a period of at or about 1 day, 2 days, 3 days, 4 days, 5 days, 6 days, 7 days, 8 days, 9 days, 10 days or 11 days.

In other embodiments, the invention provides the method described in any of the preceding paragraphs as applicable above modified such that the first period in step (b) and the second period in step (c) are each individually performed within a period of at or about 1 day, 2 days, 3 days, 4 days, 5 days, 6 days, or 7 days.

In other embodiments, the invention provides the method described in any of the preceding paragraphs as applicable above modified such that the first period in step (b) and the second period in step (c) are each individually performed within a period of at or about 5 days, 6 days, or 7 days.

In other embodiments, the invention provides the method described in any of the preceding paragraphs as applicable above modified such that the first period in step (b) and the second period in step (c) are each individually performed within a period of at or about 7 days.

In other embodiments, the invention provides the method described in any of the preceding paragraphs as applicable above modified such that steps (a) through (d) are performed in a total of at or about 14 days to at or about 18 days.

In other embodiments, the invention provides the method described in any of the preceding paragraphs as applicable above modified such that steps (a) through (d) are performed in a total of at or about 15 days to at or about 18 days.

In other embodiments, the invention provides the method described in any of the preceding paragraphs as applicable above modified such that steps (a) through (d) are performed in a total of at or about 16 days to at or about 18 days.

In other embodiments, the invention provides the method described in any of the preceding paragraphs as applicable above modified such that steps (a) through (d) are performed in a total of at or about 17 days to at or about 18 days.

In other embodiments, the invention provides the method described in any of the preceding paragraphs as applicable above modified such that steps (a) through (d) are performed in a total of at or about 14 days to at or about 17 days.

In other embodiments, the invention provides the method described in any of the preceding paragraphs as applicable above modified such that steps (a) through (d) are performed in a total of at or about 15 days to at or about 17 days.

In other embodiments, the invention provides the method described in any of the preceding paragraphs as applicable above modified such that steps (a) through (d) are performed in a total of at or about 16 days to at or about 17 days.

In other embodiments, the invention provides the method described in any of the preceding paragraphs as applicable above modified such that steps (a) through (d) are performed in a total of at or about 14 days to at or about 16 days.

In other embodiments, the invention provides the method described in any of the preceding paragraphs as applicable above modified such that steps (a) through (d) are performed in a total of at or about 15 days to at or about 16 days.

In other embodiments, the invention provides the method described in any of the preceding paragraphs as applicable above modified such that steps (a) through (d) are performed in a total of at or about 14 days.

In other embodiments, the invention provides the method described in any of the preceding paragraphs as applicable above modified such that steps (a) through (d) are performed in a total of at or about 15 days.

In other embodiments, the invention provides the method described in any of the preceding paragraphs as applicable above modified such that steps (a) through (d) are performed in a total of at or about 16 days.

In other embodiments, the invention provides the method described in any of the preceding paragraphs as applicable above modified such that steps (a) through (d) are performed in a total of at or about 17 days.

In other embodiments, the invention provides the method described in any of the preceding paragraphs as applicable above modified such that steps (a) through (d) are performed in a total of at or about 18 days.

In other embodiments, the invention provides the method described in any of the preceding paragraphs as applicable above modified such that steps (a) through (d) are performed in a total of at or about 14 days or less.

In other embodiments, the invention provides the method described in any of the preceding paragraphs as applicable above modified such that steps (a) through (d) are performed in a total of at or about 15 days or less.

In other embodiments, the invention provides the method described in any of the preceding paragraphs as applicable above modified such that steps (a) through (d) are performed in a total of at or about 16 days or less.

In other embodiments, the invention provides the method described in any of the preceding paragraphs as applicable above modified such that steps (a) through (d) are performed in a total of at or about 18 days or less.

In other embodiments, the invention provides the method described in any of the preceding paragraphs as applicable above modified such that the therapeutic population of TILs harvested in step (d) comprises sufficient TILs for a therapeutically effective dosage of the TILs.

In other embodiments, the invention provides the method described in any of the preceding paragraphs as applicable above modified such that the number of TILs sufficient for a therapeutically effective dosage is from at or about 2.3×10¹⁰ to at or about 13.7×10¹⁰.

In other embodiments, the invention provides the method described in any of the preceding paragraphs as applicable above modified such that the third population of TILs in step (c) provides for increased efficacy, increased interferon-gamma production, and/or increased polyclonality.

In other embodiments, the invention provides the method described in any of the preceding paragraphs as applicable above modified such that the third population of TILs in step (c) provides for at least a one-fold to five-fold or more interferon-gamma production as compared to TILs prepared by a process longer than 16 days.

In other embodiments, the invention provides the method described in any of the preceding paragraphs as applicable above modified such that the third population of TILs in step (c) provides for at least a one-fold to five-fold or more interferon-gamma production as compared to TILs prepared by a process longer than 17 days.

In other embodiments, the invention provides the method described in any of the preceding paragraphs as applicable above modified such that the third population of TILs in step (c) provides for at least a one-fold to five-fold or more interferon-gamma production as compared to TILs prepared by a process longer than 18 days.

In other embodiments, the invention provides the method described in any of the preceding paragraphs as applicable above modified such that the effector T cells and/or central memory T cells obtained from the third population of TILs step (c) exhibit increased CD8 and CD28 expression relative to effector T cells and/or central memory T cells obtained from the second population of cells step (b).

In other embodiments, the invention provides the method described in any of the preceding paragraphs as applicable above modified such that each container recited in the method is a closed container.

In other embodiments, the invention provides the method described in any of the preceding paragraphs as applicable above modified such that each container recited in the method is a G-container.

In other embodiments, the invention provides the method described in any of the preceding paragraphs as applicable above modified such that each container recited in the method is a GREX-10.

In other embodiments, the invention provides the method described in any of the preceding paragraphs as applicable above modified such that each container recited in the method is a GREX-100.

In other embodiments, the invention provides the method described in any of the preceding paragraphs as applicable above modified such that each container recited in the method is a GREX-500.

In some embodiments, the invention provides the method described in any of the preceding paragraphs as applicable above modified such that the at least one immunomodulatory composition comprises a membrane anchor fused to an immunomodulatory agent selected from the group consisting of: IL-2, IL-7, IL-12, IL-15, IL-18, IL-21, IL-23, IL-27, IL-4, IL-1α, IL-1β, IL-5, IFNγ, TNF α (TNFa), IFNα, IFNβ, GM-CSF, GCSF, CD40 agonist (e.g., a CD40L or an agonistic CD40 binding domain) and biologically active variants thereof. In some embodiments, the immunomodulatory agent is selected from the group consisting of IL-2, IL-7, IL-10, IL-12, IL-15, IL-18, IL-21 and a CD40 agonist. In some embodiments, the immunomodulatory agent is selected from the group consisting of IL-2, IL-12, IL-15, IL-18, IL-21, and a CD40 agonist. In some embodiments, the immunomodulatory agent is selected from the group consisting of IL-12, IL-15, IL-18, IL-21, and a CD40 agonist. In some embodiments, the immunomodulatory agent is IL-12. In some embodiments, the immunomodulatory agent is IL-15. In some embodiments, the immunomodulatory agent is IL-18. In some embodiments, the immunomodulatory agent is IL-21. In some embodiments, the immunomodulatory agent is a CD40 agonist (e.g., a CD40L or an agonistic CD40 binding domain).

In some embodiments, the invention provides the method described in any of the preceding paragraphs as applicable above modified such that the gene-editor is a transient gene-editor, wherein the TILs are transiently gene-edited to transiently express at least one immunomodulatory composition. In some embodiments, the immunomodulatory composition comprises an immunomodulatory fusion protein. In some embodiments, the immunomodulatory fusion protein comprises a membrane anchor fused to an immunomodulatory agent. In some embodiments, the immunomodulatory agent is a cytokine. In some embodiments, the cytokine is selected from the group consisting of IL-2, IL-7, IL-10, IL-12, IL-15, IL-18 and IL-21. In some embodiments, the cytokine is selected from the group consisting of IL-2, IL-12, IL-15, IL-18 and IL-21. In some embodiments, the cytokine is selected from the group consisting of IL-12, IL-15, IL-18 and IL-21. In some embodiments, the cytokine is IL-12. In some embodiments, the cytokine is IL-15. In some embodiments, the cytokine is IL-18. In some embodiments, the cytokine is IL-21. In some embodiments, the immunomodulatory agent is a CD40 agonist (e.g., a CD40L or an agonistic CD40 binding domain).

In other embodiments, the invention provides the therapeutic population of tumor infiltrating lymphocytes (TILs) made by the method described in any of the preceding paragraphs as applicable above.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added antigen-presenting cells (APCs) or OKT3, and wherein a plurality of cells in the therapeutic population of TILs include at least one immunomodulatory composition at the cell surface.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added antigen-presenting cells (APCs), and wherein a plurality of cells in the therapeutic population of TILs include at least one immunomodulatory composition at the cell surface.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed without any added OKT3, and wherein a plurality of cells in the therapeutic population of TILs include at least one immunomodulatory composition at the cell surface.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process in which the first expansion of TILs is performed with no added antigen-presenting cells (APCs) and no added OKT3, and wherein a plurality of cells in the therapeutic population of TILs include at least one immunomodulatory composition at the cell surface.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 16 days, and wherein a plurality of cells in the therapeutic population of TILs include at least one immunomodulatory composition at the cell surface.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 17 days, and wherein a plurality of cells in the therapeutic population of TILs include at least one immunomodulatory composition at the cell surface.

In other embodiments, the invention provides a therapeutic population of tumor infiltrating lymphocytes (TILs) prepared from tumor tissue of a patient, wherein the therapeutic population of TILs provides for increased efficacy, increased interferon-gamma production, and/or increased polyclonality compared to TILs prepared by a process by a process longer than 18 days, and wherein a plurality of cells in the therapeutic population of TILs include at least one immunomodulatory composition at the cell surface.

In other embodiments, the invention provides for the therapeutic population of TILs described in any of the preceding paragraphs as applicable above that provides for increased interferon-gamma production.

In other embodiments, the invention provides for the therapeutic population of TILs described in any of the preceding paragraphs as applicable above that provides for increased polyclonality.

In other embodiments, the invention provides for the therapeutic population of TILs described in any of the preceding paragraphs as applicable above that provides for increased efficacy.

In other embodiments, the invention provides for the therapeutic population of TILs described in any of the preceding paragraphs as applicable above modified such that the therapeutic population of TILs is capable of at least one-fold more interferon-gamma production as compared to TILs prepared by a process longer than 16 days. In other embodiments, the invention provides for the therapeutic population of TILs described in any of the preceding paragraphs as applicable above modified such that the therapeutic population of TILs is capable of at least one-fold more interferon-gamma production as compared to TILs prepared by a process longer than 17 days. In other embodiments, the invention provides for the therapeutic population of TILs described in any of the preceding paragraphs as applicable above modified such that the therapeutic population of TILs is capable of at least one-fold more interferon-gamma production as compared to TILs prepared by a process longer than 18 days. In some embodiments, the TILs are rendered capable of the at least one-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides for the therapeutic population of TILs described in any of the preceding paragraphs as applicable above modified such that the therapeutic population of TILs is capable of at least two-fold more interferon-gamma production as compared to TILs prepared by a process longer than 16 days. In other embodiments, the invention provides for the therapeutic population of TILs described in any of the preceding paragraphs as applicable above modified such that the therapeutic population of TILs is capable of at least two-fold more interferon-gamma production as compared to TILs prepared by a process longer than 17 days. In other embodiments, the invention provides for the therapeutic population of TILs described in any of the preceding paragraphs as applicable above modified such that the therapeutic population of TILs is capable of at least two-fold more interferon-gamma production as compared to TILs prepared by a process longer than 18 days. In some embodiments, the TILs are rendered capable of the at least two-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides for the therapeutic population of TILs described in any of the preceding paragraphs as applicable above modified such that the therapeutic population of TILs is capable of at least three-fold more interferon-gamma production as compared to TILs prepared by a process longer than 16 days. In other embodiments, the invention provides for the therapeutic population of TILs described in any of the preceding paragraphs as applicable above modified such that the therapeutic population of TILs is capable of at least three-fold more interferon-gamma production as compared to TILs prepared by a process longer than 17 days. In other embodiments, the invention provides for the therapeutic population of TILs described in any of the preceding paragraphs as applicable above modified such that the therapeutic population of TILs is capable of at least three-fold more interferon-gamma production as compared to TILs prepared by a process longer than 18 days. In some embodiments, the TILs are rendered capable of the at least three-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides for a therapeutic population of tumor infiltrating lymphocytes (TILs) that is capable of at least one-fold more interferon-gamma production as compared to TILs prepared by a process in which the first expansion of TILs is performed without any added antigen-presenting cells (APCs), and wherein a plurality of cells in the therapeutic population of TILs include at least one immunomodulatory composition at the cell surface. In some embodiments, the TILs are rendered capable of the at least one-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides for a therapeutic population of tumor infiltrating lymphocytes (TILs) that is capable of at least one-fold more interferon-gamma production as compared to TILs prepared by a process in which the first expansion of TILs is performed without any added OKT3, and wherein a plurality of cells in the therapeutic population of TILs include at least one immunomodulatory composition at the cell surface. In some embodiments, the TILs are rendered capable of the at least one-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides for a therapeutic population of TILs that is capable of at least two-fold more interferon-gamma production as compared to TILs prepared by a process in which the first expansion of TILs is performed without any added APCs, and wherein a plurality of cells in the therapeutic population of TILs include at least one immunomodulatory composition at the cell surface. In some embodiments, the TILs are rendered capable of the at least two-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides for a therapeutic population of TILs that is capable of at least two-fold more interferon-gamma production as compared to TILs prepared by a process in which the first expansion of TILs is performed without any added OKT3, and wherein a plurality of cells in the therapeutic population of TILs include at least one immunomodulatory composition at the cell surface. In some embodiments, the TILs are rendered capable of the at least two-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides for a therapeutic population of TILs that is capable of at least three-fold more interferon-gamma production as compared to TILs prepared by a process in which the first expansion of TILs is performed without any added APCs, and wherein a plurality of cells in the therapeutic population of TILs include at least one immunomodulatory composition at the cell surface. In some embodiments, the TILs are rendered capable of the at least one-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides for a therapeutic population of TILs that is capable of at least three-fold more interferon-gamma production as compared to TILs prepared by a process in which the first expansion of TILs is performed without any added OKT3, and wherein a plurality of cells in the therapeutic population of TILs include at least one immunomodulatory composition at the cell surface. In some embodiments, the TILs are rendered capable of the at least three-fold more interferon-gamma production due to the expansion process described herein, for example as described in Steps A through F above or according to Steps A through F above (also as shown, for example, in FIG. 8 (in particular, e.g., FIG. 8A and/or FIG. 8B and/or FIG. 8C and/or FIG. 8D).

In other embodiments, the invention provides the method described in any of the preceding paragraphs as applicable above modified such that the tumor fragments are small biopsies (including, for example, a punch biopsy), core biopsies, core needle biopsies or fine needle aspirates.

In other embodiments, the invention provides the method described in any of the preceding paragraphs as applicable above modified such that the tumor fragments are core biopsies.

In other embodiments, the invention provides the method described in any of the preceding paragraphs as applicable above modified such that the tumor fragments are fine needle aspirates.

In other embodiments, the invention provides the method described in any of the preceding paragraphs as applicable above modified such that the tumor fragments are small biopsies (including, for example, a punch biopsy).

In other embodiments, the invention provides the method described in any of the preceding paragraphs as applicable above modified such that the tumor fragments are core needle biopsies.

In other embodiments, the invention provides the method described in any of the preceding paragraphs as applicable above modified such that (i) the method comprises obtaining the first population of TILs from one or more small biopsies (including, for example, a punch biopsy), core biopsies, core needle biopsies or fine needle aspirates of tumor tissue from the subject, (ii) the method comprises performing the step of culturing the first population of TILs in a cell culture medium comprising IL-2 for a period of about 3 days prior to performing the step of the priming first expansion, (iii) the method comprises performing the priming first expansion for a period of about 8 days, and (iv) the method comprises performing the rapid second expansion for a period of about 11 days. In some of the foregoing embodiments, the steps of the method are completed in about 22 days.

In other embodiments, the invention provides the method described in any of the preceding paragraphs as applicable above modified such that (i) the method comprises obtaining the first population of TILs from one or more small biopsies (including, for example, a punch biopsy), core biopsies, core needle biopsies or fine needle aspirates of tumor tissue from the subject, (ii) the method comprises performing the step of culturing the first population of TILs in a cell culture medium comprising IL-2 for a period of about 3 days prior to performing the step of the priming first expansion, (iii) the method comprises performing the priming first expansion for a period of about 8 days, and (iv) the method comprises performing the rapid second expansion by culturing the culture of the second population of TILs for about 5 days, splitting the culture into up to 5 subcultures and culturing the subcultures for about 6 days. In some of the foregoing embodiments, the up to 5 subcultures are each cultured in a container that is the same size or larger than the container in which the culture of the second population of TILs is commenced in the rapid second expansion. In some of the foregoing embodiments, the culture of the second population of TILs is equally divided amongst the up to 5 subcultures. In some of the foregoing embodiments, the steps of the method are completed in about 22 days.

In other embodiments, the invention provides the method described in any of the preceding paragraphs as applicable above modified such that the first population of TILs is obtained from 1 to about 20 small biopsies (including, for example, a punch biopsy), core biopsies, core needle biopsies or fine needle aspirates of tumor tissue from the subject.

In other embodiments, the invention provides the method described in any of the preceding paragraphs as applicable above modified such that the first population of TILs is obtained from 1 to about 10 small biopsies (including, for example, a punch biopsy), core biopsies, core needle biopsies or fine needle aspirates of tumor tissue from the subject.

In other embodiments, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 small biopsies (including, for example, a punch biopsy), core biopsies, core needle biopsies or fine needle aspirates of tumor tissue from the subject.

In other embodiments, the invention provides the method described in any of the preceding paragraphs as applicable above modified such that the first population of TILs is obtained from 1, 2, 3, 4, 5, 6, 7, 8, 9 or 10 small biopsies (including, for example, a punch biopsy), core biopsies, core needle biopsies or fine needle aspirates of tumor tissue from the subject.

In other embodiments, the invention provides the method described in any of the preceding paragraphs as applicable above modified such that the first population of TILs is obtained from 1 to about 20 core biopsies of tumor tissue from the subject.

In other embodiments, the invention provides the method described in any of the preceding paragraphs as applicable above modified such that the first population of TILs is obtained from 1 to about 10 core biopsies of tumor tissue from the subject.

In other embodiments, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 core biopsies of tumor tissue from the subject.

In other embodiments, the invention provides the method described in any of the preceding paragraphs as applicable above modified such that the first population of TILs is obtained from 1, 2, 3, 4, 5, 6, 7, 8, 9 or 10 core biopsies of tumor tissue from the subject.

In other embodiments, the invention provides the method described in any of the preceding paragraphs as applicable above modified such that the first population of TILs is obtained from 1 to about 20 fine needle aspirates of tumor tissue from the subject.

In other embodiments, the invention provides the method described in any of the preceding paragraphs as applicable above modified such that the first population of TILs is obtained from 1 to about 10 fine needle aspirates of tumor tissue from the subject.

In other embodiments, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 fine needle aspirates of tumor tissue from the subject.

In other embodiments, the invention provides the method described in any of the preceding paragraphs as applicable above modified such that the first population of TILs is obtained from 1, 2, 3, 4, 5, 6, 7, 8, 9 or 10 fine needle aspirates of tumor tissue from the subject.

In other embodiments, the invention provides the method described in any of the preceding paragraphs as applicable above modified such that the first population of TILs is obtained from 1 to about 20 core needle biopsies of tumor tissue from the subject.

In other embodiments, the invention provides the method described in any of the preceding paragraphs as applicable above modified such that the first population of TILs is obtained from 1 to about 10 core needle biopsies of tumor tissue from the subject.

In other embodiments, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 core needle biopsies of tumor tissue from the subject.

In other embodiments, the invention provides the method described in any of the preceding paragraphs as applicable above modified such that the first population of TILs is obtained from 1, 2, 3, 4, 5, 6, 7, 8, 9 or 10 core needle biopsies of tumor tissue from the subject.

In other embodiments, the invention provides the method described in any of the preceding paragraphs as applicable above modified such that the first population of TILs is obtained from 1 to about 20 small biopsies (including, for example, a punch biopsy) of tumor tissue from the subject.

In other embodiments, the invention provides the method described in any of the preceding paragraphs as applicable above modified such that the first population of TILs is obtained from 1 to about 10 small biopsies (including, for example, a punch biopsy) of tumor tissue from the subject.

In other embodiments, the invention provides the method described in any of the preceding paragraphs as applicable above modified such that the first population of TILs is obtained from 1, 2, 3, 4, 5, 6, 7, 8, 9, 10, 11, 12, 13, 14, 15, 16, 17, 18, 19 or 20 small biopsies (including, for example, a punch biopsy) of tumor tissue from the subject.

In other embodiments, the invention provides the method described in any of the preceding paragraphs as applicable above modified such that the first population of TILs is obtained from 1, 2, 3, 4, 5, 6, 7, 8, 9 or 10 small biopsies (including, for example, a punch biopsy) of tumor tissue from the subject.

In other embodiments, the invention provides the method described in any of the preceding paragraphs as applicable above modified such that (i) the method comprises obtaining the first population of TILs from 1 to about 10 core biopsies of tumor tissue from the subject, (ii) the method comprises performing the step of culturing the first population of TILs in a cell culture medium comprising IL-2 for a period of about 3 days prior to performing the step of the priming first expansion, (iii) the method comprises performing the priming first expansion step by culturing the first population of TILs in a culture medium comprising IL-2, OKT-3 and antigen presenting cells (APCs) for a period of about 8 days to obtain the second population of TILs, and (iv) the method comprises performing the rapid second expansion step by culturing the second population of TILs in a culture medium comprising IL-2, OKT-3 and APCs for a period of about 11 days. In some of the foregoing embodiments, the steps of the method are completed in about 22 days.

In other embodiments, the invention provides the method described in any of the preceding paragraphs as applicable above modified such that (i) the method comprises obtaining the first population of TILs from 1 to about 10 core biopsies of tumor tissue from the subject, (ii) the method comprises performing the step of culturing the first population of TILs in a cell culture medium comprising IL-2 for a period of about 3 days prior to performing the step of the priming first expansion, (iii) the method comprises performing the priming first expansion step by culturing the first population of TILs in a culture medium comprising IL-2, OKT-3 and antigen presenting cells (APCs) for a period of about 8 days to obtain the second population of TILs, and (iv) the method comprises performing the rapid second expansion by culturing the culture of the second population of TILs in a culture medium comprising IL-2, OKT-3 and APCs for about 5 days, splitting the culture into up to 5 subcultures and culturing each of the subcultures in a culture medium comprising IL-2 for about 6 days. In some of the foregoing embodiments, the up to 5 subcultures are each cultured in a container that is the same size or larger than the container in which the culture of the second population of TILs is commenced in the rapid second expansion. In some of the foregoing embodiments, the culture of the second population of TILs is equally divided amongst the up to 5 subcultures. In some of the foregoing embodiments, the steps of the method are completed in about 22 days.

In other embodiments, the invention provides the method described in any of the preceding paragraphs as applicable above modified such that (i) the method comprises obtaining the first population of TILs from 1 to about 10 core biopsies of tumor tissue from the subject, (ii) the method comprises performing the step of culturing the first population of TILs in a cell culture medium comprising 6000 IU IL-2/mL in 0.5 L of CM1 culture medium in a G-REX-100M flask for a period of about 3 days prior to performing the step of the priming first expansion, (iii) the method comprises performing the priming first expansion by adding 0.5 L of CM1 culture medium containing 6000 IU/mL IL-2, 30 ng/mL OKT-3, and about 108 feeder cells and culturing for a period of about 8 days, and (iv) the method comprises performing the rapid second expansion by (a) transferring the second population of TILs to a G-REX-500MCS flask containing 5 L of CM2 culture medium with 3000 IU/mL IL-2, 30 ng/mL OKT-3, and 5×10⁹ feeder cells and culturing for about 5 days (b) splitting the culture into up to 5 subcultures by transferring 10⁹ TILs into each of up to 5 G-REX-500MCS flasks containing 5 L of AIM-V medium with 3000 IU/mL IL-2, and culturing the subcultures for about 6 days. In some of the foregoing embodiments, the steps of the method are completed in about 22 days.

In other embodiments, the invention provides a method of expanding T cells comprising: (a) performing a priming first expansion of a first population of T cells obtained from a donor by culturing the first population of T cells to effect growth and to prime an activation of the first population of T cells; (b) after the activation of the first population of T cells primed in step (a) begins to decay, performing a rapid second expansion of the first population of T cells by culturing the first population of T cells to effect growth and to boost the activation of the first population of T cells to obtain a second population of T cells; (c) harvesting the second population of T cells; and (d) at any time prior to or after step (c), gene-editing a portion of the T cells to express at least one immunomodulatory composition on the surface of the T cells. In other embodiments, the step of rapid second expansion is split into a plurality of steps to achieve a scaling up of the culture by: (a) performing the rapid second expansion by culturing the first population of T cells in a small scale culture in a first container, e.g., a G-REX-100MCS container, for a period of about 3 to 4 days, and then (b) effecting the transfer of the first population of T cells from the small scale culture to a second container larger than the first container, e.g., a G-REX-500MCS container, and culturing the first population of T cells from the small scale culture in a larger scale culture in the second container for a period of about 4 to 7 days. In other embodiments, the step of rapid expansion is split into a plurality of steps to achieve a scaling out of the culture by: (a) performing the rapid second expansion by culturing the first population of T cells in a first small scale culture in a first container, e.g., a G-REX-100MCS container, for a period of about 3 to 4 days, and then (b) effecting the transfer and apportioning of the first population of T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the first population of T cells from first small scale culture transferred to such second container is cultured in a second small scale culture for a period of about 4 to 7 days. In other embodiments, the step of rapid expansion is split into a plurality of steps to achieve a scaling out and scaling up of the culture by: (a) performing the rapid second expansion by culturing the first population of T cells in a small scale culture in a first container, e.g., a G-REX-100MCS container, for a period of about 3 to 4 days, and then (b) effecting the transfer and apportioning of the first population of T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX-500MCS containers, wherein in each second container the portion of the first population of T cells from the small scale culture transferred to such second container is cultured in a larger scale culture for a period of about 4 to 7 days. In other embodiments, the step of rapid expansion is split into a plurality of steps to achieve a scaling out and scaling up of the culture by: (a) performing the rapid second expansion by culturing the first population of T cells in a small scale culture in a first container, e.g., a G-REX-100MCS container, for a period of about 4 days, and then (b) effecting the transfer and apportioning of the first population of T cells from the small scale culture into and amongst 2, 3 or 4 second containers that are larger in size than the first container, e.g., G-REX-500MCS containers, wherein in each second container the portion of the first population of T cells from the small scale culture transferred to such second container is cultured in a larger scale culture for a period of about 5 days.

In some embodiments, the invention provides the method described in any of the preceding paragraphs as applicable above modified such that the at least one immunomodulatory composition comprises an immunomodulatory agent fused to a membrane anchor (e.g., a membrane anchored immunomodulatory fusion protein described herein). In some embodiments, the immunomodulatory agent is selected from the group consisting of IL-2, IL-12, IL-15, IL-18, IL-21, and a CD40 agonist (e.g., a CD40L or an agonistic CD40 binding domain).

In other embodiments, the invention provides the method described in any of the preceding paragraphs as applicable above modified such that the step of rapid second expansion is split into a plurality of steps to achieve a scaling up of the culture by: (a) performing the rapid second expansion by culturing the first population of T cells in a small scale culture in a first container, e.g., a G-REX-100MCS container, for a period of about 2 to 4 days, and then (b) effecting the transfer of the first population of T cells from the small scale culture to a second container larger than the first container, e.g., a G-REX-500MCS container, and culturing the first population of T cells from the small scale culture in a larger scale culture in the second container for a period of about 5 to 7 days.

In other embodiments, the invention provides the method described in any of the preceding paragraphs as applicable above modified such that the step of rapid expansion is split into a plurality of steps to achieve a scaling out of the culture by: (a) performing the rapid second expansion by culturing the first population of T cells in a first small scale culture in a first container, e.g., a G-REX-100MCS container, for a period of about 2 to 4 days, and then (b) effecting the transfer and apportioning of the first population of T cells from the first small scale culture into and amongst at least 2, 3, 4, 5, 6, 7, 8, 9, 10, 11, 12, 13, 14, 15, 16, 17, 18, 19, or 20 second containers that are equal in size to the first container, wherein in each second container the portion of the first population of T cells from first small scale culture transferred to such second container is cultured in a second small scale culture for a period of about 5 to 7 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX-100MCS container, for a period of about 2 to 4 days, and then (b) effecting the transfer and apportioning of the first population of T cells from the small scale culture into and amongst at least 2, 3, 4, 5, 6, 7, 8, 9, 10, 11, 12, 13, 14, 15, 16, 17, 18, 19, or 20 second containers that are larger in size than the first container, e.g., G-REX-500MCS containers, wherein in each second container the portion of the first population of T cells from the small scale culture transferred to such second container is cultured in a larger scale culture for a period of about 5 to 7 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX-100MCS container, for a period of about 3 to 4 days, and then (b) effecting the transfer and apportioning of the first population of T cells from the small scale culture into and amongst 2, 3 or 4 second containers that are larger in size than the first container, e.g., G-REX-500MCS containers, wherein in each second container the portion of the first population of T cells from the small scale culture transferred to such second container is cultured in a larger scale culture for a period of about 5 to 6 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX-100MCS container, for a period of about 3 to 4 days, and then (b) effecting the transfer and apportioning of the first population of T cells from the small scale culture into and amongst 2, 3 or 4 second containers that are larger in size than the first container, e.g., G-REX-500MCS containers, wherein in each second container the portion of the first population of T cells from the small scale culture transferred to such second container is cultured in a larger scale culture for a period of about 5 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX-100MCS container, for a period of about 3 to 4 days, and then (b) effecting the transfer and apportioning of the first population of T cells from the small scale culture into and amongst 2, 3 or 4 second containers that are larger in size than the first container, e.g., G-REX-500MCS containers, wherein in each second container the portion of the first population of T cells from the small scale culture transferred to such second container is cultured in a larger scale culture for a period of about 6 days.

In other embodiments, the invention provides the method described in any of the preceding paragraphs as applicable above modified such that the step of rapid expansion is split into a plurality of steps to achieve a scaling out and scaling up of the culture by: (a) performing the rapid second expansion by culturing the first population of T cells in a small scale culture in a first container, e.g., a G-REX-100MCS container, for a period of about 3 to 4 days, and then (b) effecting the transfer and apportioning of the first population of T cells from the small scale culture into and amongst 2, 3 or 4 second containers that are larger in size than the first container, e.g., G-REX-500MCS containers, wherein in each second container the portion of the first population of T cells from the small scale culture transferred to such second container is cultured in a larger scale culture for a period of about 7 days.

In other embodiments, the invention provides the method described in any of the preceding paragraphs as applicable above modified such that the priming first expansion of step (a) is performed during a period of up to 7 days.

In other embodiments, the invention provides the method described in any of the preceding paragraphs as applicable above modified such that the rapid second expansion of step (b) is performed during a period of up to 8 days.

In other embodiments, the invention provides the method described in any of the preceding paragraphs as applicable above modified such that the rapid second expansion of step (b) is performed during a period of up to 9 days.

In other embodiments, the invention provides the method described in any of the preceding paragraphs as applicable above modified such that the rapid second expansion of step (b) is performed during a period of up to 10 days.

In other embodiments, the invention provides the method described in any of the preceding paragraphs as applicable above modified such that the rapid second expansion of step (b) is performed during a period of up to 11 days.

In other embodiments, the invention provides the method described in any of the preceding paragraphs as applicable above modified such that the priming first expansion in step (a) is performed during a period of 7 days and the rapid second expansion of step (b) is performed during a period of up to 9 days.

In other embodiments, the invention provides the method described in any of the preceding paragraphs as applicable above modified such that the priming first expansion in step (a) is performed during a period of 7 days and the rapid second expansion of step (b) is performed during a period of up to 10 days.

In other embodiments, the invention provides the method described in any of the preceding paragraphs as applicable above modified such that the priming first expansion in step (a) is performed during a period of 7 days or 8 days and the rapid second expansion of step (b) is performed during a period of up to 9 days.

In other embodiments, the invention provides the method described in any of the preceding paragraphs as applicable above modified such that the priming first expansion in step (a) is performed during a period of 7 days or 8 days and the rapid second expansion of step (b) is performed during a period of up to 10 days.

In other embodiments, the invention provides the method described in any of the preceding paragraphs as applicable above modified such that the priming first expansion in step (a) is performed during a period of 8 days and the rapid second expansion of step (b) is performed during a period of up to 9 days.

In other embodiments, the invention provides the method described in any of the preceding paragraphs as applicable above modified such that the priming first expansion in step (a) is performed during a period of 8 days and the rapid second expansion of step (b) is performed during a period of up to 8 days.

In other embodiments, the invention provides the method described in any of the preceding paragraphs as applicable above modified such that in step (a) the first population of T cells is cultured in a first culture medium comprising OKT-3 and IL-2.

In other embodiments, the invention provides the method described in any of the preceding paragraphs as applicable above modified such that the first culture medium comprises 4-1BB agonist, OKT-3 and IL-2.

In other embodiments, the invention provides the method described in any of the preceding paragraphs as applicable above modified such that the first culture medium comprises OKT-3, IL-2 and antigen-presenting cells (APCs).

In other embodiments, the invention provides the method described in any of the preceding paragraphs as applicable above modified such that the first culture medium comprises 4-1BB agonist, OKT-3, IL-2 and antigen-presenting cells (APCs).

In other embodiments, the invention provides the method described in any of the preceding paragraphs as applicable above modified such that in step (b) the first population of T cells is cultured in a second culture medium comprising OKT-3, IL-2 and antigen-presenting cells (APCs).

In other embodiments, the invention provides the method described in any of the preceding paragraphs as applicable above modified such that the second culture medium comprises 4-1BB agonist, OKT-3, IL-2 and antigen-presenting cells (APCs).

In other embodiments, the invention provides the method described in any of the preceding paragraphs as applicable above modified such that in step (a) the first population of T cells is cultured in a first culture medium in a container comprising a first gas-permeable surface, wherein the first culture medium comprises OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In other embodiments, the invention provides the method described in any of the preceding paragraphs as applicable above modified such that in step (a) the first population of T cells is cultured in a first culture medium in a container comprising a first gas-permeable surface, wherein the first culture medium comprises 4-1BB agonist, OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In other embodiments, the invention provides the method described in any of the preceding paragraphs as applicable above modified such that in step (a) the first population of T cells is cultured in a first culture medium in a container comprising a first gas-permeable surface, wherein the first culture medium comprises OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises 4-1BB agonist, OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In other embodiments, the invention provides the method described in any of the preceding paragraphs as applicable above modified such that in step (a) the first population of T cells is cultured in a first culture medium in a container comprising a first gas-permeable surface, wherein the first culture medium comprises 4-1BB agonist, OKT-3, IL-2 and a first population of antigen-presenting cells (APCs), wherein the first population of APCs is exogenous to the donor of the first population of T cells and the first population of APCs is layered onto the first gas-permeable surface, wherein in step (b) the first population of T cells is cultured in a second culture medium in the container, wherein the second culture medium comprises 4-1BB agonist, OKT-3, IL-2 and a second population of APCs, wherein the second population of APCs is exogenous to the donor of the first population of T cells and the second population of APCs is layered onto the first gas-permeable surface, and wherein the second population of APCs is greater than the first population of APCs.

In other embodiments, the invention provides the method described in any of the preceding paragraphs as applicable above modified such that the ratio of the number of APCs in the second population of APCs to the number of APCs in the first population of APCs is about 2:1.

In other embodiments, the invention provides the method described in any of the preceding paragraphs as applicable above modified such that the number of APCs in the first population of APCs is about 2.5×10⁸ and the number of APCs in the second population of APCs is about 5×10⁸.

In other embodiments, the invention provides the method described in any of the preceding paragraphs as applicable above modified such that in step (a) the first population of APCs is layered onto the first gas-permeable surface at an average thickness of 2 layers of APCs.

In other embodiments, the invention provides the method described in any of the preceding paragraphs as applicable above modified such that in step (b) the second population of APCs is layered onto the first gas-permeable surface at an average thickness selected from the range of 4 to 8 layers of APCs.

In other embodiments, the invention provides the method described in any of the preceding paragraphs as applicable above modified such that the ratio of the average number of layers of APCs layered onto the first gas-permeable surface in step (b) to the average number of layers of APCs layered onto the first gas-permeable surface in step (a) is 2:1.

In other embodiments, the invention provides the method described in any of the preceding paragraphs as applicable above modified such that in step (a) the first population of APCs is seeded on the first gas permeable surface at a density selected from the range of at or about 1.0×10⁶ APCs/cm² to at or about 4.5×10⁶ APCs/cm².

In other embodiments, the invention provides the method described in any of the preceding paragraphs as applicable above modified such that in step (a) the first population of APCs is seeded on the first gas permeable surface at a density selected from the range of at or about 1.5×10⁶ APCs/cm² to at or about 3.5×10⁶ APCs/cm².

In other embodiments, the invention provides the method described in any of the preceding paragraphs as applicable above modified such that in step (a) the first population of APCs is seeded on the first gas permeable surface at a density selected from the range of at or about 2.0×10⁶ APCs/cm² to at or about 3.0×10⁶ APCs/cm².

In other embodiments, the invention provides the method described in any of the preceding paragraphs as applicable above modified such that in step (a) the first population of APCs is seeded on the first gas permeable surface at a density of at or about 2.0×10⁶ APCs/cm².

In other embodiments, the invention provides the method described in any of the preceding paragraphs as applicable above modified such that in step (b) the second population of APCs is seeded on the first gas permeable surface at a density selected from the range of at or about 2.5×10⁶ APCs/cm² to at or about 7.5×10⁶ APCs/cm².

In other embodiments, the invention provides the method described in any of the preceding paragraphs as applicable above modified such that in step (b) the second population of APCs is seeded on the first gas permeable surface at a density selected from the range of at or about 3.5×10⁶ APCs/cm² to at or about 6.0×10⁶ APCs/cm².

In other embodiments, the invention provides the method described in any of the preceding paragraphs as applicable above modified such that in step (b) the second population of APCs is seeded on the first gas permeable surface at a density selected from the range of at or about 4.0×10⁶ APCs/cm² to at or about 5.5×10⁶ APCs/cm².

In other embodiments, the invention provides the method described in any of the preceding paragraphs as applicable above modified such that in step (b) the second population of APCs is seeded on the first gas permeable surface at a density of at or about 4.0×10⁶ APCs/cm².

In other embodiments, the invention provides the method described in any of the preceding paragraphs as applicable above modified such that in step (a) the first population of APCs is seeded on the first gas permeable surface at a density selected from the range of at or about 1.0×10⁶ APCs/cm² to at or about 4.5×10⁶ APCs/cm² and in step (b) the second population of APCs is seeded on the first gas permeable surface at a density selected from the range of at or about 2.5×10⁶ APCs/cm² to at or about 7.5×10⁶ APCs/cm².

In other embodiments, the invention provides the method described in any of the preceding paragraphs as applicable modified such that in step (a) the first population of APCs is seeded on the first gas permeable surface at a density selected from the range of at or about 1.5×10⁶ APCs/cm² to at or about 3.5×10⁶ APCs/cm² and in step (b) the second population of APCs is seeded on the first gas permeable surface at a density selected from the range of at or about 3.5×10⁶ APCs/cm² to at or about 6.0×10⁶ APCs/cm².

In other embodiments, the invention provides the method described in any of the preceding paragraphs as applicable above modified such that in step (a) the first population of APCs is seeded on the first gas permeable surface at a density selected from the range of at or about 2.0×10⁶ APCs/cm² to at or about 3.0×10⁶ APCs/cm² and in step (b) the second population of APCs is seeded on the first gas permeable surface at a density selected from the range of at or about 4.0×10⁶ APCs/cm² to at or about 5.5×10⁶ APCs/cm².

In other embodiments, the invention provides the method described in any of the preceding paragraphs as applicable above modified such that in step (a) the first population of APCs is seeded on the first gas permeable surface at a density of at or about 2.0×10⁶ APCs/cm² and in step (b) the second population of APCs is seeded on the first gas permeable surface at a density of at or about 4.0×10⁶ APCs/cm².

In other embodiments, the invention provides the method described in any of the preceding paragraphs as applicable above modified such that the APCs are peripheral blood mononuclear cells (PBMCs).

In other embodiments, the invention provides the method described in any of the preceding paragraphs as applicable above modified such that the PBMCs are irradiated and exogenous to the donor of the first population of T cells.

In other embodiments, the invention provides the method described in any of the preceding paragraphs as applicable above modified such that the T cells are tumor infiltrating lymphocytes (TILs).

In other embodiments, the invention provides the method described in any of the preceding paragraphs as applicable above modified such that the T cells are marrow infiltrating lymphocytes (MILs).

In other embodiments, the invention provides the method described in any of the preceding paragraphs as applicable above modified such that the T cells are peripheral blood lymphocytes (PBLs).

In other embodiments, the invention provides the method described in any of the preceding paragraphs as applicable above modified such that the first population of T cells is obtained by separation from the whole blood of the donor.

In other embodiments, the invention provides the method described in any of the preceding paragraphs as applicable above modified such that the first population of T cells is obtained by separation from the apheresis product of the donor.

In other embodiments, the invention provides the method described in any of the preceding paragraphs as applicable above modified such that the first population of T cells is separated from the whole blood or apheresis product of the donor by positive or negative selection of a T cell phenotype.

In other embodiments, the invention provides the method described in any of the preceding paragraphs as applicable above modified such that the T cell phenotype is CD3+ and CD45+.

In other embodiments, the invention provides the method described in any of the preceding paragraphs as applicable above modified such that before performing the priming first expansion of the first population of T cells the T cells are separated from NK cells. In other embodiments, the T cells are separated from NK cells in the first population of T cells by removal of CD3− CD56+ cells from the first population of T cells. In other embodiments, the CD3− CD56+ cells are removed from the first population of T cells by subjecting the first population of T cells to cell sorting using a gating strategy that removes the CD3− CD56+ cell fraction and recovers the negative fraction. In other embodiments, the foregoing method is utilized for the expansion of T cells in a first population of T cells characterized by a high percentage of NK cells. In other embodiments, the foregoing method is utilized for the expansion of T cells in a first population of T cells characterized by a high percentage of CD3− CD56+ cells. In other embodiments, the foregoing method is utilized for the expansion of T cells in tumor tissue characterized by the present of a high number of NK cells. In other embodiments, the foregoing method is utilized for the expansion of T cells in tumor tissue characterized by a high number of CD3− CD56+ cells. In other embodiments, the foregoing method is utilized for the expansion of T cells in tumor tissue obtained from a patient suffering from a tumor characterized by the presence of a high number of NK cells. In other embodiments, the foregoing method is utilized for the expansion of T cells in tumor tissue obtained from a patient suffering from a tumor characterized by the presence of a high number of CD3− CD56+ cells. In other embodiments, the foregoing method is utilized for the expansion of T cells in tumor tissue obtained from a patient suffering from ovarian cancer.

In other embodiments, the invention provides the method described in any of the preceding paragraphs as applicable above modified such that at or about 1×10⁷ T cells from the first population of T cells are seeded in a container to initiate the primary first expansion culture in such container.

In other embodiments, the invention provides the method described in any of the preceding paragraphs as applicable above modified such that the first population of T cells is distributed into a plurality of containers, and in each container at or about 1×10⁷ T cells from the first population of T cells are seeded to initiate the primary first expansion culture in such container.

In other embodiments, the invention provides the method described in any of the preceding paragraphs as applicable above modified such that the second population of T cells harvested in step (c) is a therapeutic population of TILs.

In other embodiments, the invention provides the method described in any of the preceding paragraphs as applicable above modified such that the first population of T cells is obtained from one or more small biopsies (including, for example, a punch biopsy), core biopsies, core needle biopsies or fine needle aspirates of tumor tissue from the donor.

In other embodiments, the invention provides the method described in any of the preceding paragraphs as applicable above modified such that the first population of T cells is obtained from 1 to 20 small biopsies (including, for example, a punch biopsy), core biopsies, core needle biopsies or fine needle aspirates of tumor tissue from the donor.

In other embodiments, the invention provides the method described in any of the preceding paragraphs as applicable above modified such that the first population of T cells is obtained from 1 to 10 small biopsies (including, for example, a punch biopsy), core biopsies, core needle biopsies or fine needle aspirates of tumor tissue from the donor.

In other embodiments, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 small biopsies (including, for example, a punch biopsy), core biopsies, core needle biopsies or fine needle aspirates of tumor tissue from the donor.

In other embodiments, the invention provides the method described in any of the preceding paragraphs as applicable above modified such that the first population of T cells is obtained from 1, 2, 3, 4, 5, 6, 7, 8, 9, or 10 small biopsies (including, for example, a punch biopsy), core biopsies, core needle biopsies or fine needle aspirates of tumor tissue from the donor.

In other embodiments, the invention provides the method described in any of the preceding paragraphs as applicable above modified such that the first population of T cells is obtained from one or more core biopsies of tumor tissue from the donor.

In other embodiments, the invention provides the method described in any of the preceding paragraphs as applicable above modified such that the first population of T cells is obtained from 1 to 20 core biopsies of tumor tissue from the donor.

In other embodiments, the invention provides the method described in any of the preceding paragraphs as applicable above modified such that the first population of T cells is obtained from 1 to 10 core biopsies of tumor tissue from the donor.

In other embodiments, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 core biopsies of tumor tissue from the donor.

In other embodiments, the invention provides the method described in any of the preceding paragraphs as applicable above modified such that the first population of T cells is obtained from 1, 2, 3, 4, 5, 6, 7, 8, 9, or 10 core biopsies of tumor tissue from the donor.

In other embodiments, the invention provides the method described in any of the preceding paragraphs as applicable above modified such that the first population of T cells is obtained from one or more fine needle aspirates of tumor tissue from the donor.

In other embodiments, the invention provides the method described in any of the preceding paragraphs as applicable above modified such that the first population of T cells is obtained from 1 to 20 fine needle aspirates of tumor tissue from the donor.

In other embodiments, the invention provides the method described in any of the preceding paragraphs as applicable above modified such that the first population of T cells is obtained from 1 to 10 fine needle aspirates of tumor tissue from the donor.

In other embodiments, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 fine needle aspirates of tumor tissue from the donor.

In other embodiments, the invention provides the method described in any of the preceding paragraphs as applicable above modified such that the first population of T cells is obtained from 1, 2, 3, 4, 5, 6, 7, 8, 9, or 10 fine needle aspirates of tumor tissue from the donor.

In other embodiments, the invention provides the method described in any of the preceding paragraphs as applicable above modified such that the first population of T cells is obtained from one or more small biopsies (including, for example, a punch biopsy) of tumor tissue from the donor.

In other embodiments, the invention provides the method described in any of the preceding paragraphs as applicable above modified such that the first population of T cells is obtained from 1 to 20 small biopsies (including, for example, a punch biopsy) of tumor tissue from the donor.

In other embodiments, the invention provides the method described in any of the preceding paragraphs as applicable above modified such that the first population of T cells is obtained from 1 to 10 small biopsies (including, for example, a punch biopsy) of tumor tissue from the donor.

In other embodiments, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 small biopsies (including, for example, a punch biopsy) of tumor tissue from the donor.

In other embodiments, the invention provides the method described in any of the preceding paragraphs as applicable above modified such that the first population of T cells is obtained from 1, 2, 3, 4, 5, 6, 7, 8, 9, or 10 small biopsies (including, for example, a punch biopsy) of tumor tissue from the donor.

In other embodiments, the invention provides the method described in any of the preceding paragraphs as applicable above modified such that the first population of T cells is obtained from one or more core needle biopsies of tumor tissue from the donor.

In other embodiments, the invention provides the method described in any of the preceding paragraphs as applicable above modified such that the first population of T cells is obtained from 1 to 20 core needle biopsies of tumor tissue from the donor.

In other embodiments, the invention provides the method described in any of the preceding paragraphs as applicable above modified such that the first population of T cells is obtained from 1 to 10 core needle biopsies of tumor tissue from the donor.

In other embodiments, the invention provides the method described in any of the preceding paragraphs as applicable above modified such that the first population of T cells is obtained from 1, 2, 3, 4, 5, 6, 7, 8, 9, 10, 11, 12, 13, 14, 15, 16, 17, 18, 19 or 20 core needle biopsies of tumor tissue from the donor.

In other embodiments, the invention provides the method described in any of the preceding paragraphs as applicable above modified such that the first population of T cells is obtained from 1, 2, 3, 4, 5, 6, 7, 8, 9, or 10 core needle biopsies of tumor tissue from the donor.

In other embodiments, the invention provides a method for expanding tumor infiltrating lymphocytes (TILs) into a therapeutic population of TILs comprising: i) obtaining and/or receiving a first population of TILs from a tumor sample obtained from one or more small biopsies, core biopsies, or needle biopsies of a tumor in a subject by culturing the tumor sample in a first cell culture medium comprising IL-2 for about 3 days; (ii) performing a priming first expansion by culturing the first population of TILs in a second cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 7 or 8 days to obtain the second population of TILs, wherein the second population of TILs is greater in number than the first population of TILs; (iii) performing a rapid second expansion by supplementing the second cell culture medium of the second population of TILs with additional IL-2, OKT-3, and APCs, to produce a third population of TILs, wherein the number of APCs added in the rapid second expansion is at least twice the number of APCs added in step (ii), wherein the rapid second expansion is performed for a second period of about 11 days to obtain the third population of TILs, wherein the third population of TILs is a therapeutic population of TILs, wherein the rapid second expansion is performed in a container comprising a second gas-permeable surface area; (iv) harvesting the therapeutic population of TILs obtained from step (iii); (v) transferring the harvested TIL population from step (iv) to an infusion bag; and (vi) at any time prior to or after step (iv), gene-editing at least a portion of the TIL cells to express at least one immunomodulatory composition on the surface of the TIL cells.

In other embodiments, the invention provides a method for expanding tumor infiltrating lymphocytes (TILs) into a therapeutic population of TILs comprising: (i) obtaining and/or receiving a first population of TILs from a tumor sample obtained from one or more small biopsies, core biopsies, or needle biopsies of a tumor in a subject by culturing the tumor sample in a first cell culture medium comprising IL-2 for about 3 days; (ii) performing a priming first expansion by culturing the first population of TILs in a second cell culture medium comprising IL-2, OKT-3, and antigen presenting cells (APCs) to produce a second population of TILs, wherein the priming first expansion is performed for first period of about 7 or 8 days to obtain the second population of TILs, wherein the second population of TILs is greater in number than the first population of TILs; (iii) performing a rapid second expansion by contacting the second population of TILs with a third cell culture medium comprising IL-2, OKT-3, and APCs, to produce a third population of TILs, wherein the rapid second expansion is performed for a second period of about 11 days to obtain the third population of TILs, wherein the third population of TILs is a therapeutic population of TILs; (iv) harvesting the therapeutic population of TILs obtained from step (iii); and (v) at any time prior to or after step (iv), gene-editing at least a portion of the TIL cells to express at least one immunomodulatory composition on the surface of the TIL cells.

In other embodiments, the invention provides the method described in any of the preceding paragraphs as applicable above modified such that after day 5 of the second period the culture is split into 2 or more subcultures, and each subculture is supplemented with an additional quantity of the third culture medium and cultured for about 6 days.

In other embodiments, the invention provides the method described in any of the preceding paragraphs as applicable above modified such that after day 5 of the second period the culture is split into 2 or more subcultures, and each subculture is supplemented with a fourth culture medium comprising IL-2 and cultured for about 6 days.

In other embodiments, the invention provides the method described in any of the preceding paragraphs as applicable above modified such that after day 5 of the second period the culture is split into up to 5 subcultures.

In other embodiments, the invention provides the method described in any of the preceding paragraphs as applicable above modified such that all steps in the method are completed in about 22 days.

In other embodiments, the invention provides a method of expanding T cells comprising: (i) performing a priming first expansion of a first population of T cells from a tumor sample obtained from one or more small biopsies, core biopsies, or needle biopsies of a tumor in a donor by culturing the first population of T cells to effect growth and to prime an activation of the first population of T cells; (ii) after the activation of the first population of T cells primed in step (a) begins to decay, performing a rapid second expansion of the first population of T cells by culturing the first population of T cells to effect growth and to boost the activation of the first population of T cells to obtain a second population of T cells; (iv) harvesting the second population of T cells; and (v) at any time prior to or after step (iv), gene-editing at least a portion of the T cells to express at least one immunomodulatory composition on the surface of the T cells. In some embodiments, the tumor sample is obtained from a plurality of core biopsies. In some embodiments, the plurality of core biopsies is selected from the group consisting of 2, 3, 4, 5, 6, 7, 8, 9 and 10 core biopsies.

In some embodiments, the invention provides the method described in any of the preceding paragraphs as applicable above modified such that the at least one immunomodulatory composition comprises a membrane anchor fused to an immunodulatory agent selected from the group consisting of: IL-2, IL-7, IL-12, IL-15, IL-18, IL-21, IL-23, IL-27, IL-4, IL-1α, IL-1β, IL-5, IFNγ, TNF α (TNFa), IFNα, IFNβ, GM-CSF, GCSF, a CD40 agonist (e.g., a CD40L or an agonistic CD40 binding domain) and biologically active variants thereof. In some embodiments, the cytokine is selected from the group consisting of IL-12, IL-15, IL-18 and IL-21. In some embodiments, the cytokine is IL-12. In some embodiments, the cytokine is IL-15. In some embodiments, the cytokine is IL-18. In some embodiments, the cytokine is IL-21. In some embodiments, the immunomodulatory agent is a CD40 agonist (e.g., a CD40L or an agonistic CD40 binding domain).

In some embodiments, the invention the method described in any of the preceding paragraphs as applicable above modified such that T cells or TILs are obtained from tumor digests. In some embodiments, tumor digests are generated by incubating the tumor in enzyme media, for example but not limited to RPMI 1640, 2 mM GlutaMAX, 10 mg/mL gentamicin, 30 U/mL DNase, and 1.0 mg/mL collagenase, followed by mechanical dissociation (GentleMACS, Miltenyi Biotec, Auburn, CA). In some embodiments, the tumor is placed in a tumor dissociating enzyme mixture including one or more dissociating (digesting) enzymes such as, but not limited to, collagenase (including any blend or type of collagenase), Accutase™, Accumax™, hyaluronidase, neutral protease (dispase), chymotrypsin, chymopapain, trypsin, caseinase, elastase, papain, protease type XIV (pronase), deoxyribonuclease I (DNase), trypsin inhibitor, any other dissociating or proteolytic enzyme, and any combination thereof. In other embodiments, the tumor is placed in a tumor dissociating enzyme mixture including collagenase (including any blend or type of collagenase), neutral protease (dispase) and deoxyribonuclease I (DNase).

### IX. Pharmaceutical Compositions, Dosages, and Dosing Regimens

In some embodiments, TILs, MILs, or PBLs expanded and/or genetically modified (including TILs, MILs, or PBLs genetically-modified to express a CCR) using the methods of the present disclosure are administered to a patient as a pharmaceutical composition. In some embodiments, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

Any suitable dose of TILs can be administered. In some embodiments, from about 2.3×10¹⁰ to about 13.7×10¹⁰ TILs are administered, with an average of around 7.8×10¹⁰ TILs, particularly if the cancer is NSCLC or melanoma. In some embodiments, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered. In some embodiments, the therapeutically effective dosage is about 2.3×10¹⁰ to about 13.7×10¹⁰. In some embodiments, the therapeutically effective dosage is about 7.8×10¹⁰ TILs, particularly of the cancer is melanoma. n some embodiments, the therapeutically effective dosage is about 7.8×10¹⁰ TILs, particularly of the cancer is NSCLC. In some embodiments, the therapeutically effective dosage is about 1.2×10¹⁰ to about 4.3×10¹⁰ of TILs. In some embodiments, the therapeutically effective dosage is about 3×10¹⁰ to about 12×10¹⁰ TILs. In some embodiments, the therapeutically effective dosage is about 4×10¹⁰ to about 10×10¹⁰ TILs. In some embodiments, the therapeutically effective dosage is about 5×10¹⁰ to about 8×10¹⁰ TILs. In some embodiments, the therapeutically effective dosage is about 6×10¹⁰ to about 8×10¹⁰ TILs. In some embodiments, the therapeutically effective dosage is about 7×10¹⁰ to about 8×10¹⁰ TILs.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰ 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%, 16%, 15%, 14%, 13%, 12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.00010% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.00010% w/w, w/v, or v/v of the pharmaceutical composition.

In some embodiments, the concentration of the TILs provided in the pharmaceutical compositions of the invention is in the range from about 0.00010% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.0010% to about 10%, about 0.010% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

In other embodiments, the invention provides an infusion bag comprising the therapeutic population of TILs described in any of the preceding paragraphs above.

In other embodiments, the invention provides a tumor infiltrating lymphocyte (TIL) composition comprising the therapeutic population of TILs described in any of the preceding paragraphs above and a pharmaceutically acceptable carrier.

In other embodiments, the invention provides an infusion bag comprising the TIL composition described in any of the preceding paragraphs above.

In other embodiments, the invention provides a cryopreserved preparation of the therapeutic population of TILs described in any of the preceding paragraphs above.

In other embodiments, the invention provides a tumor infiltrating lymphocyte (TIL) composition comprising the therapeutic population of TILs described in any of the preceding paragraphs above and a cryopreservation media.

In other embodiments, the invention provides the TIL composition described in any of the preceding paragraphs above modified such that the cryopreservation media contains DMSO.

In other embodiments, the invention provides the TIL composition described in any of the preceding paragraphs above modified such that the cryopreservation media contains 7-10% DMSO.

In other embodiments, the invention provides a cryopreserved preparation of the TIL composition described in any of the preceding paragraphs above.

In some embodiments, TILs expanded using the methods of the present disclosure are administered to a patient as a pharmaceutical composition. In some embodiments, the pharmaceutical composition is a suspension of TILs in a sterile buffer. TILs expanded using PBMCs of the present disclosure may be administered by any suitable route as known in the art. In some embodiments, the T-cells are administered as a single intra-arterial or intravenous infusion, which preferably lasts approximately 30 to 60 minutes. Other suitable routes of administration include intraperitoneal, intrathecal, and intralymphatic administration.

Any suitable dose of TILs can be administered. In some embodiments, from about 2.3×10¹⁰ to about 13.7×10¹⁰ TILs are administered, with an average of around 7.8×10¹⁰ TILs, particularly if the cancer is NSCLC. In some embodiments, about 1.2×10¹⁰ to about 4.3×10¹⁰ of TILs are administered. In some embodiments, about 3×10¹⁰ to about 12×10¹⁰ TILs are administered. In some embodiments, about 4×10¹⁰ to about 10×10¹⁰ TILs are administered. In some embodiments, about 5×10¹⁰ to about 8×10¹⁰ TILs are administered. In some embodiments, about 6×10¹⁰ to about 8×10¹⁰ TILs are administered. In some embodiments, about 7×10¹⁰ to about 8×10¹⁰ TILs are administered. In some embodiments, therapeutically effective dosage is about 2.3×10¹⁰ to about 13.7×10¹⁰. In some embodiments, therapeutically effective dosage is about 7.8×10¹⁰ TILs, particularly of the cancer is NSCLC. In some embodiments, therapeutically effective dosage is about 1.2×10¹⁰ to about 4.3×10¹⁰ of TILs. In some embodiments, therapeutically effective dosage is about 3×10¹⁰ to about 12×10¹⁰ TILs. In some embodiments, therapeutically effective dosage is about 4×10¹⁰ to about 10×10¹⁰ TILs. In some embodiments, therapeutically effective dosage is about 5×10¹⁰ to about 8×10¹⁰ TILs. In some embodiments, therapeutically effective dosage is about 6×10¹⁰ to about 8×10¹⁰ TILs. In some embodiments, therapeutically effective dosage is about 7×10¹⁰ to about 8×10¹⁰ TILs.

In some embodiments, the number of the TILs provided in the pharmaceutical compositions of the invention is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, the number of the TILs provided in the pharmaceutical compositions of the invention is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, the concentration of the TILs provided in the pharmaceutical compositions of the invention is less than, for example, 100%, 90%, 80%, 70%, 60%, 50%, 40%, 30%, 20%, 19%, 18%, 17%1, 16%1, 15%, 14%, 13%, %12%, 11%, 10%, 9%, 8%, 7%, 6%, 5%, 4%, 3%, 2%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.00010% w/w, w/v or v/v of the pharmaceutical composition.

In some embodiments, the concentration of the TILs provided in the pharmaceutical compositions of the invention is greater than 90%, 80%, 70%, 60%, 50%, 40%, 30%, 20%, 19.75%, 19.50%, 19.25% 19%, 18.75%, 18.50%, 18.25% 18%, 17.75%, 17.50%, 17.25% 17%, 16.75%, 16.50%, 16.25% 16%, 15.75%, 15.50%, 15.25% 15%, 14.75%, 14.50%, 14.25% 14%, 13.75%, 13.50%, 13.25% 13%, 12.75%, 12.50%, 12.25% 12%, 11.75%, 11.50%, 11.25% 11%, 10.75%, 10.50%, 10.25% 10%, 9.75%, 9.50%, 9.25% 9%, 8.75%, 8.50%, 8.25% 8%, 7.75%, 7.50%, 7.25% 7%, 6.75%, 6.50%, 6.25% 6%, 5.75%, 5.50%, 5.25% 5%, 4.75%, 4.50%, 4.25%, 4%, 3.75%, 3.50%, 3.25%, 3%, 2.75%, 2.50%, 2.25%, 2%, 1.75%, 1.50%, 125%, 1%, 0.5%, 0.4%, 0.3%, 0.2%, 0.1%, 0.09%, 0.08%, 0.07%, 0.06%, 0.05%, 0.04%, 0.03%, 0.02%, 0.01%, 0.009%, 0.008%, 0.007%, 0.006%, 0.005%, 0.004%, 0.003%, 0.002%, 0.001%, 0.0009%, 0.0008%, 0.0007%, 0.0006%, 0.0005%, 0.0004%, 0.0003%, 0.0002% or 0.0001% w/w, w/v, or v/v of the pharmaceutical composition.

In some embodiments, the concentration of the TILs provided in the pharmaceutical compositions of the invention is in the range from about 0.0001% to about 50%, about 0.001% to about 40%, about 0.01% to about 30%, about 0.02% to about 29%, about 0.03% to about 28%, about 0.04% to about 27%, about 0.05% to about 26%, about 0.06% to about 25%, about 0.07% to about 24%, about 0.08% to about 23%, about 0.09% to about 22%, about 0.1% to about 21%, about 0.2% to about 20%, about 0.3% to about 19%, about 0.4% to about 18%, about 0.5% to about 17%, about 0.6% to about 16%, about 0.7% to about 15%, about 0.8% to about 14%, about 0.9% to about 12% or about 1% to about 10% w/w, w/v or v/v of the pharmaceutical composition.

In some embodiments, the concentration of the TILs provided in the pharmaceutical compositions of the invention is in the range from about 0.001% to about 10%, about 0.01% to about 5%, about 0.02% to about 4.5%, about 0.03% to about 4%, about 0.04% to about 3.5%, about 0.05% to about 3%, about 0.06% to about 2.5%, about 0.07% to about 2%, about 0.08% to about 1.5%, about 0.09% to about 1%, about 0.1% to about 0.9% w/w, w/v or v/v of the pharmaceutical composition.

In some embodiments, the amount of the TILs provided in the pharmaceutical compositions of the invention is equal to or less than 10 g, 9.5 g, 9.0 g, 8.5 g, 8.0 g, 7.5 g, 7.0 g, 6.5 g, 6.0 g, 5.5 g, 5.0 g, 4.5 g, 4.0 g, 3.5 g, 3.0 g, 2.5 g, 2.0 g, 1.5 g, 1.0 g, 0.95 g, 0.9 g, 0.85 g, 0.8 g, 0.75 g, 0.7 g, 0.65 g, 0.6 g, 0.55 g, 0.5 g, 0.45 g, 0.4 g, 0.35 g, 0.3 g, 0.25 g, 0.2 g, 0.15 g, 0.1 g, 0.09 g, 0.08 g, 0.07 g, 0.06 g, 0.05 g, 0.04 g, 0.03 g, 0.02 g, 0.01 g, 0.009 g, 0.008 g, 0.007 g, 0.006 g, 0.005 g, 0.004 g, 0.003 g, 0.002 g, 0.001 g, 0.0009 g, 0.0008 g, 0.0007 g, 0.0006 g, 0.0005 g, 0.0004 g, 0.0003 g, 0.0002 g, or 0.0001 g.

In some embodiments, the amount of the TILs provided in the pharmaceutical compositions of the invention is more than 0.0001 g, 0.0002 g, 0.0003 g, 0.0004 g, 0.0005 g, 0.0006 g, 0.0007 g, 0.0008 g, 0.0009 g, 0.001 g, 0.0015 g, 0.002 g, 0.0025 g, 0.003 g, 0.0035 g, 0.004 g, 0.0045 g, 0.005 g, 0.0055 g, 0.006 g, 0.0065 g, 0.007 g, 0.0075 g, 0.008 g, 0.0085 g, 0.009 g, 0.0095 g, 0.01 g, 0.015 g, 0.02 g, 0.025 g, 0.03 g, 0.035 g, 0.04 g, 0.045 g, 0.05 g, 0.055 g, 0.06 g, 0.065 g, 0.07 g, 0.075 g, 0.08 g, 0.085 g, 0.09 g, 0.095 g, 0.1 g, 0.15 g, 0.2 g, 0.25 g, 0.3 g, 0.35 g, 0.4 g, 0.45 g, 0.5 g, 0.55 g, 0.6 g, 0.65 g, 0.7 g, 0.75 g, 0.8 g, 0.85 g, 0.9 g, 0.95 g, 1 g, 1.5 g, 2 g, 2.5, 3 g, 3.5, 4 g, 4.5 g, 5 g, 5.5 g, 6 g, 6.5 g, 7 g, 7.5 g, 8 g, 8.5 g, 9 g, 9.5 g, or 10 g.

The TILs provided in the pharmaceutical compositions of the invention are effective over a wide dosage range. The exact dosage will depend upon the route of administration, the form in which the compound is administered, the gender and age of the subject to be treated, the body weight of the subject to be treated, and the preference and experience of the attending physician. The clinically-established dosages of the TILs may also be used if appropriate. The amounts of the pharmaceutical compositions administered using the methods herein, such as the dosages of TILs, will be dependent on the human or mammal being treated, the severity of the disorder or condition, the rate of administration, the disposition of the active pharmaceutical ingredients and the discretion of the prescribing physician.

In some embodiments, TILs may be administered in a single dose. Such administration may be by injection, e.g., intravenous injection. In some embodiments, TILs may be administered in multiple doses. Dosing may be once, twice, three times, four times, five times, six times, or more than six times per year. Dosing may be once a month, once every two weeks, once a week, or once every other day. Administration of TILs may continue as long as necessary.

In some embodiments, an effective dosage of TILs is about 1×10⁶, 2×10⁶, 3×10⁶, 4×10⁶, 5×10⁶, 6×10⁶, 7×10⁶, 8×10⁶, 9×10⁶, 1×10⁷, 2×10⁷, 3×10⁷, 4×10⁷, 5×10⁷, 6×10⁷, 7×10⁷, 8×10⁷, 9×10⁷, 1×10⁸, 2×10⁸, 3×10⁸, 4×10⁸, 5×10⁸, 6×10⁸, 7×10⁸, 8×10⁸, 9×10⁸, 1×10⁹, 2×10⁹, 3×10⁹, 4×10⁹, 5×10⁹, 6×10⁹, 7×10⁹, 8×10⁹, 9×10⁹, 1×10¹⁰, 2×10¹⁰, 3×10¹⁰, 4×10¹⁰, 5×10¹⁰, 6×10¹⁰, 7×10¹⁰, 8×10¹⁰, 9×10¹⁰, 1×10¹¹, 2×10¹¹, 3×10¹¹, 4×10¹¹, 5×10¹¹, 6×10¹¹, 7×10¹¹, 8×10¹¹, 9×10¹¹, 1×10¹², 2×10¹², 3×10¹², 4×10¹², 5×10¹², 6×10¹², 7×10¹², 8×10¹², 9×10¹², 1×10¹³, 2×10¹³, 3×10¹³, 4×10¹³, 5×10¹³, 6×10¹³, 7×10¹³, 8×10¹³, and 9×10¹³. In some embodiments, an effective dosage of TILs is in the range of 1×10⁶ to 5×10⁶, 5×10⁶ to 1×10⁷, 1×10⁷ to 5×10⁷, 5×10⁷ to 1×10⁸, 1×10⁸ to 5×10⁸, 5×10⁸ to 1×10⁹, 1×10⁹ to 5×10⁹, 5×10⁹ to 1×10¹⁰, 1×10¹⁰ to 5×10¹⁰, 5×10¹⁰ to 1×10¹¹, 5×10¹¹ to 1×10¹², 1×10¹² to 5×10¹², and 5×10¹² to 1×10¹³.

In some embodiments, an effective dosage of TILs is in the range of about 0.01 mg/kg to about 4.3 mg/kg, about 0.15 mg/kg to about 3.6 mg/kg, about 0.3 mg/kg to about 3.2 mg/kg, about 0.35 mg/kg to about 2.85 mg/kg, about 0.15 mg/kg to about 2.85 mg/kg, about 0.3 mg to about 2.15 mg/kg, about 0.45 mg/kg to about 1.7 mg/kg, about 0.15 mg/kg to about 1.3 mg/kg, about 0.3 mg/kg to about 1.15 mg/kg, about 0.45 mg/kg to about 1 mg/kg, about 0.55 mg/kg to about 0.85 mg/kg, about 0.65 mg/kg to about 0.8 mg/kg, about 0.7 mg/kg to about 0.75 mg/kg, about 0.7 mg/kg to about 2.15 mg/kg, about 0.85 mg/kg to about 2 mg/kg, about 1 mg/kg to about 1.85 mg/kg, about 1.15 mg/kg to about 1.7 mg/kg, about 1.3 mg/kg mg to about 1.6 mg/kg, about 1.35 mg/kg to about 1.5 mg/kg, about 2.15 mg/kg to about 3.6 mg/kg, about 2.3 mg/kg to about 3.4 mg/kg, about 2.4 mg/kg to about 3.3 mg/kg, about 2.6 mg/kg to about 3.15 mg/kg, about 2.7 mg/kg to about 3 mg/kg, about 2.8 mg/kg to about 3 mg/kg, or about 2.85 mg/kg to about 2.95 mg/kg.

In some embodiments, an effective dosage of TILs is in the range of about 1 mg to about 500 mg, about 10 mg to about 300 mg, about 20 mg to about 250 mg, about 25 mg to about 200 mg, about 1 mg to about 50 mg, about 5 mg to about 45 mg, about 10 mg to about 40 mg, about 15 mg to about 35 mg, about 20 mg to about 30 mg, about 23 mg to about 28 mg, about 50 mg to about 150 mg, about 60 mg to about 140 mg, about 70 mg to about 130 mg, about 80 mg to about 120 mg, about 90 mg to about 110 mg, or about 95 mg to about 105 mg, about 98 mg to about 102 mg, about 150 mg to about 250 mg, about 160 mg to about 240 mg, about 170 mg to about 230 mg, about 180 mg to about 220 mg, about 190 mg to about 210 mg, about 195 mg to about 205 mg, or about 198 to about 207 mg.

An effective amount of the TILs may be administered in either single or multiple doses by any of the accepted modes of administration of agents having similar utilities, including intranasal and transdermal routes, by intra-arterial injection, intravenously, intraperitoneally, parenterally, intramuscularly, subcutaneously, topically, by transplantation, or by inhalation.

### X. Methods of Treating Patients

Methods of treatment begin with the initial TIL collection and culture of TILs. Such methods have been both described in the art by, for example, Jin et al., J. Immunotherapy, 2012, 35(3):283-292, incorporated by reference herein in its entirety. Embodiments of methods of treatment are described throughout the sections below, including the Examples.

The expanded TILs produced according the methods described herein, including for example as described in Steps A through F above or according to Steps A through F above (also as shown, for example, in FIG. 1 and or FIG. 8) find particular use in the treatment of patients with cancer (for example, as described in Goff, et al., J. Clinical Oncology, 2016, 34(20):2389-239, as well as the supplemental content; incorporated by reference herein in its entirety. In some embodiments, TIL were grown from resected deposits of metastatic melanoma as previously described (see, Dudley, et al., J Immunother., 2003, 26:332-342; incorporated by reference herein in its entirety). Fresh tumor can be dissected under sterile conditions. A representative sample can be collected for formal pathologic analysis. Single fragments of 2 mm³ to 3 mm³ may be used. In some embodiments, 5, 10, 15, 20, 25 or 30 samples per patient are obtained. In some embodiments, 20, 25, or 30 samples per patient are obtained. In some embodiments, 20, 22, 24, 26, or 28 samples per patient are obtained. In some embodiments, 24 samples per patient are obtained. Samples can be placed in individual wells of a 24-well plate, maintained in growth media with high-dose IL-2 (6,000 IU/mL), and monitored for destruction of tumor and/or proliferation of TIL. Any tumor with viable cells remaining after processing can be enzymatically digested into a single cell suspension and cryopreserved, as described herein.

In some embodiments, successfully grown TIL can be sampled for phenotype analysis (CD3, CD4, CD8, and CD56) and tested against autologous tumor when available. TIL can be considered reactive if overnight coculture yielded interferon-gamma (IFN-γ) levels >200 pg/mL and twice background. (Goff, et al., J Immunother., 2010, 33:840-847; incorporated by reference herein in its entirety). In some embodiments, cultures with evidence of autologous reactivity or sufficient growth patterns can be selected for a second expansion, (for example, a second expansion as provided in according to Step D of FIG. 1 and/or FIG. 8), including second expansions that are sometimes referred to as rapid expansion (REP). In some embodiments, expanded TILs with high autologous reactivity (for example, high proliferation during a second expansion), are selected for an additional second expansion. In some embodiments, TILs with high autologous reactivity (for example, high proliferation during second expansion as provided in Step D of FIG. 1 and/or FIG. 8), are selected for an additional second expansion according to Step D of FIG. 1 and/or FIG. 8.

Cell phenotypes of cryopreserved samples of infusion bag TIL can be analyzed by flow cytometry (e.g., FlowJo) for surface markers CD3, CD4, CD8, CCR7, and CD45RA (BD BioSciences), as well as by any of the methods described herein. Serum cytokines were measured by using standard enzyme-linked immunosorbent assay techniques. A rise in serum IFN-g was defined as >100 pg/mL and greater than 4 3 baseline levels.

In some embodiments, the TILs produced by the methods provided herein, for example those exemplified in FIG. 1 and/or FIG. 8, provide for a surprising improvement in clinical efficacy of the TILs. In some embodiments, the TILs produced by the methods provided herein, for example those exemplified in FIG. 1 and/or FIG. 8, exhibit increased clinical efficacy as compared to TILs produced by methods other than those described herein, including for example, methods other than those exemplified in FIG. 1 and/or FIG. 8. In some embodiments, the methods other than those described herein include methods referred to as process 1C and/or Generation 1 (Gen 1). In some embodiments, the increased efficacy is measured by DCR, ORR, and/or other clinical responses. In some embodiments, the TILs produced by the methods provided herein, for example those exemplified in FIG. 1, exhibit a similar time to response and safety profile compared to TILs produced by methods other than those described herein, including for example, methods other than those exemplified in FIG. 1 and/or FIG. 8.

In some embodiments, IFN-gamma (IFN-γ) is indicative of treatment efficacy and/or increased clinical efficacy. In some embodiments, IFN-γ in the blood of subjects treated with TILs is indicative of active TILs. In some embodiments, a potency assay for IFN-γ production is employed. IFN-γ production is another measure of cytotoxic potential. IFN-γ production can be measured by determining the levels of the cytokine IFN-γ in the blood, serum, or TILs ex vivo of a subject treated with TILs prepared by the methods of the present invention, including those as described for example in FIG. 1 and/or FIG. 8. In some embodiments, an increase in IFN-γ is indicative of treatment efficacy in a patient treated with the TILs produced by the methods of the present invention. In some embodiments, IFN-γ is increased one-fold, two-fold, three-fold, four-fold, or five-fold or more as compared to an untreated patient and/or as compared to a patient treated with TILs prepared using other methods than those provide herein including for example, methods other than those embodied in FIG. 1 and/or FIG. 8. In some embodiments, IFN-γ secretion is increased one-fold as compared to an untreated patient and/or as compared to a patient treated with TILs prepared using other methods than those provide herein including for example, methods other than those embodied in FIG. 1 and/or FIG. 8. In some embodiments, IFN-γ secretion is increased two-fold as compared to an untreated patient and/or as compared to a patient treated with TILs prepared using other methods than those provide herein including for example, methods other than those embodied in FIG. 1 and/or FIG. 8. In some embodiments, IFN-γ secretion is increased three-fold as compared to an untreated patient and/or as compared to a patient treated with TILs prepared using other methods than those provide herein including for example, methods other than those embodied in FIG. 1 and/or FIG. 8. In some embodiments, IFN-γ secretion is increased four-fold as compared to an untreated patient and/or as compared to a patient treated with TILs prepared using other methods than those provide herein including for example, methods other than those embodied in FIG. 1 and/or FIG. 8. In some embodiments, IFN-γ secretion is increased five-fold as compared to an untreated patient and/or as compared to a patient treated with TILs prepared using other methods than those provide herein including for example, methods other than those embodied in FIG. 1 and/or FIG. 8. In some embodiments, IFN-γ is measured using a Quantikine ELISA kit. In some embodiments, IFN-γ is measured in TILs ex vivo of a subject treated with TILs prepared by the methods of the present invention, including those as described for example in FIG. 1 and/or FIG. 8. In some embodiments, IFN-γ is measured in blood of a subject treated with TILs prepared by the methods of the present invention, including those as described for example in FIG. 1 and/or FIG. 8. In some embodiments, IFN-γ is measured in TILs serum of a subject treated with TILs prepared by the methods of the present invention, including those as described for example in FIG. 1 and/or FIG. 8. In some embodiments, IFN-gamma (IFN-γ) is indicative of treatment efficacy and/or increased clinical efficacy in the treatment of cancer.

In some embodiments, the TILs prepared by the methods of the present invention, including those as described for example in FIG. 1 in some embodiments, IFN-gamma (IFN-γ) is indicative of treatment efficacy and/or increased clinical efficacy. In some embodiments, IFN-γ in the blood of subjects treated with TILs is indicative of active TILs. In some embodiments, a potency assay for IFN-γ production is employed. IFN-γ production is another measure of cytotoxic potential. IFN-γ production can be measured by determining the levels of the cytokine IFN-γ in the blood, serum, or TILs ex vivo of a subject treated with TILs prepared by the methods of the present invention, including those as described for example in FIG. 1 and/or FIG. 8. In some embodiments, an increase in IFN-γ is indicative of treatment efficacy in a patient treated with the TILs produced by the methods of the present invention. In some embodiments, IFN-γ is increased one-fold, two-fold, three-fold, four-fold, or five-fold or more IFN-γ as compared to an untreated patient and/or as compared to a patient treated with TILs prepared using other methods than those provide herein including for example, methods other than those embodied in FIG. 1 and/or FIG. 8.

In some embodiments, the TILs prepared by the methods of the present invention, including those as described for example in FIG. 1 and/or FIG. 8, exhibit increased polyclonality as compared to TILs produced by other methods, including those not exemplified in FIG. 1 and/or FIG. 8, including for example, methods referred to as process 1C methods. In some embodiments, significantly improved polyclonality and/or increased polyclonality is indicative of treatment efficacy and/or increased clinical efficacy. In some embodiments, polyclonality refers to the T-cell repertoire diversity. In some embodiments, an increase in polyclonality can be indicative of treatment efficacy with regard to administration of the TILs produced by the methods of the present invention. In some embodiments, polyclonality is increased one-fold, two-fold, ten-fold, 100-fold, 500-fold, or 1000-fold as compared to TILs prepared using methods than those provide herein including for example, methods other than those embodied in FIG. 1 and/or FIG. 8. In some embodiments, polyclonality is increased one-fold as compared to an untreated patient and/or as compared to a patient treated with TILs prepared using other methods than those provide herein including for example, methods other than those embodied in FIG. 1 and/or FIG. 8. In some embodiments, polyclonality is increased two-fold as compared to an untreated patient and/or as compared to a patient treated with TILs prepared using other methods than those provide herein including for example, methods other than those embodied in FIG. 1 and/or FIG. 8. In some embodiments, polyclonality is increased ten-fold as compared to an untreated patient and/or as compared to a patient treated with TILs prepared using other methods than those provide herein including for example, methods other than those embodied in FIG. 1 and/or FIG. 8. In some embodiments, polyclonality is increased 100-fold as compared to an untreated patient and/or as compared to a patient treated with TILs prepared using other methods than those provide herein including for example, methods other than those embodied in FIG. 1 and/or FIG. 8. In some embodiments, polyclonality is increased 500-fold as compared to an untreated patient and/or as compared to a patient treated with TILs prepared using other methods than those provide herein including for example, methods other than those embodied in FIG. 1 and/or FIG. 8. In some embodiments, polyclonality is increased 1000-fold as compared to an untreated patient and/or as compared to a patient treated with TILs prepared using other methods than those provide herein including for example, methods other than those embodied in FIG. 1 and/or FIG. 8.

Measures of efficacy can include the disease control rate (DCR) as well as overall response rate (ORR), as known in the art as well as described herein.

### A. Methods of Treating Cancers

The compositions and methods described herein can be used in a method for treating diseases. In some embodiments, they are for use in treating hyperproliferative disorders, such as cancer, in an adult patient or in a pediatric patient. They may also be used in treating other disorders as described herein and in the following paragraphs.

In some embodiments, the hyperproliferative disorder is cancer. In some embodiments, the hyperproliferative disorder is a solid tumor cancer. In some embodiments, the solid tumor cancer is selected from the group consisting of anal cancer, bladder cancer, breast cancer (including triple-negative breast cancer), bone cancer, cancer caused by human papilloma virus (HPV), central nervous system associated cancer (including ependymoma, medulloblastoma, neuroblastoma, pineoblastoma, and primitive neuroectodermal tumor), cervical cancer (including squamous cell cervical cancer, adenosquamous cervical cancer, and cervical adenocarcinoma), colon cancer, colorectal cancer, endometrial cancer, esophageal cancer, esophagogastric junction cancer, gastric cancer, gastrointestinal cancer, gastrointestinal stromal tumor, glioblastoma, glioma, head and neck cancer (including head and neck squamous cell carcinoma (HNSCC), hypopharynx cancer, larynx cancer, nasopharynx cancer, oropharynx cancer, and pharynx cancer), kidney cancer, liver cancer, lung cancer (including non-small-cell lung cancer (NSCLC) and small-cell lung cancer), melanoma (including uveal melanoma, choroidal melanoma, ciliary body melanoma, or iris melanoma), mesothelioma (including malignant pleural mesothelioma), ovarian cancer, pancreatic cancer (including pancreatic ductal adenocarcinoma), penile cancer, rectal cancer, renal cancer, renal cell carcinoma, sarcoma (including Ewing sarcoma, osteosarcoma, rhabdomyosarcoma, and other bone and soft tissue sarcomas), thyroid cancer (including anaplastic thyroid cancer), uterine cancer, and vaginal cancer.

In some embodiments, the hyperproliferative disorder is a hematological malignancy. In some embodiments, the hematological malignancy is selected from the group consisting of chronic lymphocytic leukemia, acute lymphoblastic leukemia, diffuse large B cell lymphoma, non-Hodgkin's lymphoma, Hodgkin's lymphoma, follicular lymphoma, mantle cell lymphoma, and multiple myeloma. In some embodiments, the present invention includes a method of treating a patient with a cancer, wherein the cancer is a hematological malignancy. In some embodiments, the present invention includes a method of treating a patient with a cancer using TILs, MILs, or PBLs modified to express one or more CCRs, wherein the cancer is a hematological malignancy. In some embodiments, the present invention includes a method of treating a patient with a cancer using MILs or PBLs modified to express one or more CCRs, wherein the cancer is a hematological malignancy.

In some embodiments, the cancer is one of the foregoing cancers, including solid tumor cancers and hematological malignancies, that is relapsed or refractory to treatment with at least one prior therapy, including chemotherapy, radiation therapy, or immunotherapy. In some embodiments, the cancer is one of the foregoing cancers that is relapsed or refractory to treatment with at least two prior therapies, including chemotherapy, radiation therapy, and/or immunotherapy. In some embodiments, the cancer is one of the foregoing cancers that is relapsed or refractory to treatment with at least three prior therapies, including chemotherapy, radiation therapy, and/or immunotherapy.

In some embodiments, the cancer is a microsatellite instability-high (MSI-H) or a mismatch repair deficient (dMMR) cancer. MSI-H and dMMR cancers and testing therefore have been described in Kawakami, et al., Curr. Treat. Options Oncol. 2015, 16, 30, the disclosures of which are incorporated by reference herein.

In some embodiments, the present invention includes a method of treating a patient with a cancer using TILs, MILs, or PBLs modified to express one or more CCRs, wherein the patient is a human. In some embodiments, the present invention includes a method of treating a patient with a cancer using TILs, MILs, or PBLs modified to express one or more CCRs, wherein the patient is a non-human. In some embodiments, the present invention includes a method of treating a patient with a cancer using TILs, MILs, or PBLs modified to express one or more CCRs, wherein the patient is a companion animal.

In some embodiments, the present invention includes a method of treating a patient with a cancer, wherein the cancer is refractory to treatment with a BRAF inhibitor and/or a MEK inhibitor. In some embodiments, the present invention includes a method of treating a patient with a cancer, wherein the cancer is refractory to treatment with a BRAF inhibitor selected from the group consisting of vemurafenib, dabrafenib, encorafenib, sorafenib, and pharmaceutically acceptable salts or solvates thereof. In some embodiments, the present invention includes a method of treating a patient with a cancer, wherein the cancer is refractory to treatment with a MEK inhibitor selected from the group consisting of trametinib, cobimetinib, binimetinib, selumetinib, pimasertinib, refametinib, and pharmaceutically acceptable salts or solvates thereof. In some embodiments, the present invention includes a method of treating a patient with a cancer, wherein the cancer is refractory to treatment with a BRAF inhibitor selected from the group consisting of vemurafenib, dabrafenib, encorafenib, sorafenib, and pharmaceutically acceptable salts or solvates thereof, and a MEK inhibitor selected from the group consisting of trametinib, cobimetinib, binimetinib, selumetinib, pimasertinib, refametinib, and pharmaceutically acceptable salts or solvates thereof.

In some embodiments, the present invention includes a method of treating a patient with a cancer, wherein the cancer is a pediatric cancer.

In some embodiments, the present invention includes a method of treating a patient with a cancer wherein the cancer is uveal melanoma.

In some embodiments, the present invention includes a method of treating a patient with a cancer, wherein the uveal melanoma is choroidal melanoma, ciliary body melanoma, or iris melanoma.

In some embodiments, the present invention includes a method of treating a patient with a cancer, wherein the pediatric cancer is a neuroblastoma.

In some embodiments, the present invention includes a method of treating a patient with a cancer, wherein the pediatric cancer is a sarcoma.

In some embodiments, the present invention includes a method of treating a patient with a cancer, wherein the sarcoma is osteosarcoma.

In some embodiments, the present invention includes a method of treating a patient with a cancer, wherein the sarcoma is a soft tissue sarcoma.

In some embodiments, the present invention includes a method of treating a patient with a cancer, wherein the soft tissue sarcoma is rhabdomyosarcoma, Ewing sarcoma, or primitive neuroectodermal tumor (PNET).

In some embodiments, the present invention includes a method of treating a patient with a cancer, wherein the pediatric cancer is a central nervous system (CNS) associated cancer. In some embodiments, the pediatric cancer is refractory to treatment with chemotherapy. In some embodiments, the pediatric cancer is refractory to treatment with radiation therapy. In some embodiments, the pediatric cancer is refractory to treatment with dinutuximab.

In some embodiments, the present invention includes a method of treating a patient with a cancer, wherein the CNS associated cancer is medulloblastoma, pineoblastoma, glioma, ependymoma, or glioblastoma.

The compositions and methods described herein can be used in a method for treating cancer, wherein the cancer is refractory or resistant to prior treatment with an anti-PD-1 or anti-PD-L1 antibody. In some embodiments, the patient is a primary refractory patient to an anti-PD-1 or anti-PD-L1 antibody. In some embodiments, the patient shows no prior response to an anti-PD-1 or anti-PD-L1 antibody. In some embodiments, the patient shows a prior response to an anti-PD-1 or anti-PD-L1 antibody, follow by progression of the patient's cancer. In some embodiments, the cancer is refractory to an anti-CTLA-4 antibody and/or an anti-PD-1 or anti-PD-L1 antibody in combination with at least one chemotherapeutic agent. In some embodiments, the prior chemotherapeutic agent is carboplatin, paclitaxel, pemetrexed, and/or cisplatin. In some prior embodiments, the chemotherapeutic agent(s) is a platinum doublet chemotherapeutic agent. In some embodiments, the platinum doublet therapy comprises a first chemotherapeutic agent selected from the group consisting of cisplatin and carboplatin and a second chemotherapeutic agent selected from the group consisting of vinorelbine, gemcitabine and a taxane (including for example, paclitaxel, docetaxel or nab-paclitaxel). In some embodiments, the platinum doublet chemotherapeutic agent is in combination with pemetrexed.

In some embodiments, the NSCLC is PD-L1 negative and/or is from a patient with a cancer that expresses PD-L1 with a tumor proportion score (TPS) of <1%, as described elsewhere herein.

In some embodiments, the NSCLC is refractory to a combination therapy comprising an anti-PD-1 or the anti-PD-L1 antibody and a platinum doublet therapy, wherein the platinum doublet therapy comprises:
i) a first chemotherapeutic agent selected from the group consisting of cisplatin and carboplatin,
ii) and a second chemotherapeutic agent selected from the group consisting of vinorelbine, gemcitabine and a taxane (including for example, paclitaxel, docetaxel or nab-paclitaxel).

In some embodiments, the NSCLC is refractory to a combination therapy comprising an anti-PD-1 or the anti-PD-L1 antibody, pemetrexed, and a platinum doublet therapy, wherein the platinum doublet therapy comprises:
i) a first chemotherapeutic agent selected from the group consisting of cisplatin and carboplatin,
ii) and a second chemotherapeutic agent selected from the group consisting of vinorelbine, gemcitabine and a taxane (including for example, paclitaxel, docetaxel or nab-paclitaxel).

In some embodiments, the NSCLC has been treated with an anti-PD-1 antibody. In some embodiments, the NSCLC has been treated with an anti-PD-L1 antibody. In some embodiments, the NSCLC patient is treatment naïve. In some embodiments, the NSCLC has not been treated with an anti-PD-1 antibody. In some embodiments, the NSCLC has not been treated with an anti-PD-L1 antibody. In some embodiments, the NSCLC has been previously treated with a chemotherapeutic agent. In some embodiments, the NSCLC has been previously treated with a chemotherapeutic agent but is not longer being treated with the chemotherapeutic agent. In some embodiments, the NSCLC patient is anti-PD-1/PD-L1 naïve. In some embodiments, the NSCLC patient has low expression of PD-L1. In some embodiments, the NSCLC patient has treatment naïve NSCLC or is post-chemotherapeutic treatment but anti-PD-1/PD-L1 naïve. In some embodiments, the NSCLC patient is treatment naïve or post-chemotherapeutic treatment but anti-PD-1/PD-L1 naïve and has low expression of PD-L1. In some embodiments, the NSCLC patient has bulky disease at baseline. In some embodiments, the subject has bulky disease at baseline and has low expression of PD-L1. In some embodiments, the NSCLC patient has no detectable expression of PD-L1. In some embodiments, the NSCLC patient is treatment naïve or post-chemotherapeutic treatment but anti-PD-1/PD-L1 naïve and has no detectable expression of PD-L1. In some embodiments, the patient has bulky disease at baseline and has no detectable expression of PD-L1. In some embodiments, the NSCLC patient has treatment naïve NSCLC or post chemotherapy (e.g., post chemotherapeutic agent) but anti-PD-1/PD-L1 naïve who have low expression of PD-L1 and/or have bulky disease at baseline. In some embodiments, bulky disease is indicated where the maximal tumor diameter is greater than 7 cm measured in either the transverse or coronal plane. In some embodiments, bulky disease is indicated when there are swollen lymph nodes with a short-axis diameter of 20 mm or greater. In some embodiments, the chemotherapeutic includes a standard of care therapeutic for NSCLC.

In some embodiments, PD-L1 expression is determined by the tumor proportion score. In some embodiments, the subject with a refractory NSCLC tumor has a <1% tumor proportion score (TPS). In some embodiments, the subject with a refractory NSCLC tumor has a ≥1% TPS. In some embodiments, subject with the refractory NSCLC has been previously treated with an anti-PD-1 and/or anti-PD-L1 antibody and the tumor proportion score was determined prior to said anti-PD-1 and/or anti-PD-L1 antibody treatment. In some embodiments, subject with the refractory NSCLC has been previously treated with an anti-PD-L1 antibody and the tumor proportion score was determined prior to said anti-PD-L1 antibody treatment.

In some embodiments, the TILs prepared by the methods of the present invention, including those as described for example in FIG. 1 or FIG. 8, exhibit increased polyclonality as compared to TILs produced by other methods, including those not exemplified in FIG. 1 or FIG. 8, such as for example, methods referred to as process 1C methods. In some embodiments, significantly improved polyclonality and/or increased polyclonality is indicative of treatment efficacy and/or increased clinical efficacy for cancer treatment. In some embodiments, polyclonality refers to the T-cell repertoire diversity. In some embodiments, an increase in polyclonality can be indicative of treatment efficacy with regard to administration of the TILs produced by the methods of the present invention. In some embodiments, polyclonality is increased one-fold, two-fold, ten-fold, 100-fold, 500-fold, or 1000-fold as compared to TILs prepared using methods than those provide herein including for example, methods other than those embodied in FIG. 1 or FIG. 8. In some embodiments, polyclonality is increased one-fold as compared to an untreated patient and/or as compared to a patient treated with TILs prepared using other methods than those provide herein including for example, methods other than those embodied in FIG. 1 or FIG. 8. In some embodiments, polyclonality is increased two-fold as compared to an untreated patient and/or as compared to a patient treated with TILs prepared using other methods than those provide herein including for example, methods other than those embodied in FIG. 1 or FIG. 8. In some embodiments, polyclonality is increased ten-fold as compared to an untreated patient and/or as compared to a patient treated with TILs prepared using other methods than those provide herein including for example, methods other than those embodied in FIG. 1 or FIG. 8. In some embodiments, polyclonality is increased 100-fold as compared to an untreated patient and/or as compared to a patient treated with TILs prepared using other methods than those provide herein including for example, methods other than those embodied in FIG. 1 or FIG. 8. In some embodiments, polyclonality is increased 500-fold as compared to an untreated patient and/or as compared to a patient treated with TILs prepared using other methods than those provide herein including for example, methods other than those embodied in FIG. 1 or FIG. 8. In some embodiments, polyclonality is increased 1000-fold as compared to an untreated patient and/or as compared to a patient treated with TILs prepared using other methods than those provide herein including for example, methods other than those embodied in FIG. 1 or FIG. 8.

In some embodiments, PD-L1 expression is determined by the tumor proportion score using one more testing methods as described herein. In some embodiments, the subject or patient with a NSCLC tumor has a <1% tumor proportion score (TPS). In some embodiments, the NSCLC tumor has a ≥1% TPS. In some embodiments, the subject or patient with the NSCLC has been previously treated with an anti-PD-1 and/or anti-PD-L1 antibody and the tumor proportion score was determined prior to the anti-PD-1 and/or anti-PD-L1 antibody treatment. In some embodiments, the subject or patient with the NSCLC has been previously treated with an anti-PD-L1 antibody and the tumor proportion score was determined prior to the anti-PD-L1 antibody treatment. In some embodiments, the subject or patient with a refractory or resistant NSCLC tumor has a <1% tumor proportion score (TPS). In some embodiments, the subject or patient with a refractory or resistant NSCLC tumor has a ≥1% TPS. In some embodiments, the subject or patient with the refractory or resistant NSCLC has been previously treated with an anti-PD-1 and/or anti-PD-L1 antibody and the tumor proportion score was determined prior to the anti-PD-1 and/or anti-PD-L1 antibody treatment. In some embodiments, the subject or patient with the refractory or resistant NSCLC has been previously treated with an anti-PD-L1 antibody and the tumor proportion score was determined prior to the anti-PD-L1 antibody treatment.

In some embodiments, the NSCLC is an NSCLC that exhibits a tumor proportion score (TPS), or the percentage of viable tumor cells from a patient taken prior to anti-PD-1 or anti-PD-L1 therapy, showing partial or complete membrane staining at any intensity, for the PD-L1 protein that is less than 1% (TPS<1%). In some embodiments, the NSCLC is an NSCLC that exhibits a TPS selected from the group consisting of <50%, <45%, <40%, <35%, <30%, <25%, <20%, <15%, <10%, <9%, <8%, <7%, <6%, <5%, <4%, <3%, <2%, <1%, <0.9%, <0.8%, <0.7%, <0.6%, <0.5%, <0.4%, <0.3%, <0.2%, <0.1%, <0.09%, <0.08%, <0.07%, <0.06%, <0.05%, <0.04%, <0.03%, <0.02%, and <0.01%. In some embodiments, the NSCLC is an NSCLC that exhibits a TPS selected from the group consisting of about 50%, about 45%, about 40%, about 35%, about 30%, about 25%, about 20%, about 15%, about 10%, about 9%, about 8%, about 7%, about 6%, about 5%, about 4%, about 3%, about 2%, about 1%, about 0.9%, about 0.8%, about 0.7%, about 0.6%, about 0.5%, about 0.4%, about 0.3%, about 0.2%, about 0.1%, about 0.09%, about 0.08%, about 0.07%, about 0.06%, about 0.05%, about 0.04%, about 0.03%, about 0.02%, and about 0.01%. In some embodiments, the NSCLC is an NSCLC that exhibits a TPS between 0% and 1%. In some embodiments, the NSCLC is an NSCLC that exhibits a TPS between 0% and 0.9%. In some embodiments, the NSCLC is an NSCLC that exhibits a TPS between 0% and 0.8%. In some embodiments, the NSCLC is an NSCLC that exhibits a TPS between 0% and 0.7%. In some embodiments, the NSCLC is an NSCLC that exhibits a TPS between 0% and 0.6%. In some embodiments, the NSCLC is an NSCLC that exhibits a TPS between 0% and 0.5%. In some embodiments, the NSCLC is an NSCLC that exhibits a TPS between 0% and 0.4%. In some embodiments, the NSCLC is an NSCLC that exhibits a TPS between 0% and 0.3%. In some embodiments, the NSCLC is an NSCLC that exhibits a TPS between 0% and 0.2%. In some embodiments, the NSCLC is an NSCLC that exhibits a TPS between 0% and 0.1%. TPS may be measured by methods known in the art, such as those described in Hirsch, et al. J Thorac. Oncol. 2017, 12, 208-222 or those used for the determination of TPS prior to treatment with pembrolizumab or other anti-PD-1 or anti-PD-L1 therapies. Methods for measurement of TPS that have been approved by the U.S. Food and Drug Administration may also be used. In some embodiments, the PD-L1 is exosomal PD-L1. In some embodiments, the PD-L1 is found on circulating tumor cells.

In some embodiments, the partial membrane staining includes 1%, 5%, 10%, 15%, 20%, 25%, 30%, 35%, 40%, 45%, 50%, 55%, 60%, 65%, 70%, 75%, 80%, 85%, 90%, 95%, 97%, 99%, or more. In some embodiments, the completed membrane staining includes approximately 100% membrane staining.

In some embodiments, testing for PD-L1 can involve measuring levels of PD-L1 in patient serum. In these embodiments, measurement of PD-L1 in patient serum removes the uncertainty of tumor heterogeneity and the patient discomfort of serial biopsies.

In some embodiments, elevated soluble PD-L1 as compared to a baseline or standard level correlates with worsened prognosis in NSCLC. See, for example, Okuma, et al., Clinical Lung Cancer, 2018, 19, 410-417; Vecchiarelli, et al., Oncotarget, 2018, 9, 17554-17563. In some embodiments, the PD-L1 is exosomal PD-L1. In some embodiments, the PD-L1 is expressed on circulating tumor cells.

In some embodiments, the invention provides a method of treating non-small cell lung carcinoma (NSCLC) by administering a population of tumor infiltrating lymphocytes (TILs) to a subject or patient in need thereof, wherein the subject or patient has at least one of:
i. a predetermined tumor proportion score (TPS) of PD-L1<1%,
ii. a TPS score of PD-L1 of 1%-49%, or
iii. a predetermined absence of one or more driver mutations,
   wherein the driver mutation is selected from the group consisting of an EGFR mutation, an EGFR insertion, an EGFR exon 20 mutation, a KRAS mutation, a BRAF mutation, an ALK mutation, a c-ROS mutation (ROS1 mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation, and wherein the method comprises:
(a) obtaining and/or receiving a first population of TILs from a tumor resected from the subject or patient by processing a tumor sample obtained from the subject into multiple tumor fragments;
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting therapeutic population of TILs obtained from step (d), wherein the transition from step (d) to step (e) occurs without opening the system; and
(f) transferring the harvested TIL population from step (e) to an infusion bag, wherein the transfer from step (e) to (f) occurs without opening the system;
(g) cryopreserving the infusion bag comprising the harvested TIL population from step (f) using a cryopreservation process; and
(h) administering a therapeutically effective dosage of the third population of TILs from the infusion bag in step (g) to the subject or patient.

In some embodiments, the invention provides a method of treating non-small cell lung carcinoma (NSCLC) by administering a population of tumor infiltrating lymphocytes (TILs) to a patient in need thereof, wherein the method comprises:
(a) testing the patient's tumor for PD-L1 expression and tumor proportion score (TPS) of PD-L1,
(b) testing the patient for the absence of one or more driver mutations, wherein the driver mutation is selected from the group consisting of an EGFR mutation, an EGFR insertion, an EGFR exon 20 mutation, a KRAS mutation, a BRAF mutation, an ALK mutation, a c-ROS mutation (ROS1 mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation,
(c) determining that the patient has a TPS score for PD-L1 of about 1% to about 49% and determining that the patient also has no driver mutations,
(d) obtaining and/or receiving a first population of TILs from a tumor resected from the subject or patient by processing a tumor sample obtained from the subject into multiple tumor fragments;
(e) adding the first population of TILs into a closed system;
(f) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (e) to step (f) occurs without opening the system;
(g) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (f) to step (g) occurs without opening the system;
(h) harvesting therapeutic population of TILs obtained from step (d), wherein the transition from step (d) to step (e) occurs without opening the system; and
(i) transferring the harvested TIL population from step (e) to an infusion bag, wherein the transfer from step (e) to (f) occurs without opening the system;
(j) cryopreserving the infusion bag comprising the harvested TIL population from step (f) using a cryopreservation process; and
(k) administering a therapeutically effective dosage of the third population of TILs from the infusion bag in step (g) to the subject or patient.

In some embodiments, the invention provides a method of treating non-small cell lung carcinoma (NSCLC) by administering a population of tumor infiltrating lymphocytes (TILs) to a patient in need thereof, wherein the method comprises:
(a) testing the patient's tumor for PD-L1 expression and tumor proportion score (TPS) of PD-L1,
(b) testing the patient for the absence of one or more driver mutations, wherein the driver mutation is selected from the group consisting of an EGFR mutation, an EGFR insertion, an EGFR exon 20 mutation, a KRAS mutation, a BRAF mutation, an ALK mutation, a c-ROS mutation (ROS1 mutation), a ROS1 fusion, a RET mutation, a RET fusion, an ERBB2 mutation, an ERBB2 amplification, a BRCA mutation, a MAP2K1 mutation, PIK3CA, CDKN2A, a PTEN mutation, an UMD mutation, an NRAS mutation, a KRAS mutation, an NF1 mutation, a MET mutation, a MET splice and/or altered MET signaling, a TP53 mutation, a CREBBP mutation, a KMT2C mutation, a KMT2D mutation, an ARID1A mutation, a RB1 mutation, an ATM mutation, a SETD2 mutation, a FLT3 mutation, a PTPN11 mutation, a FGFR1 mutation, an EP300 mutation, a MYC mutation, an EZH2 mutation, a JAK2 mutation, a FBXW7 mutation, a CCND3 mutation, and a GNA11 mutation,
(c) determining that the patient has a TPS score for PD-L1 of less than about 1% and determining that the patient also has no driver mutations,
(d) obtaining and/or receiving a first population of TILs from a tumor resected from the subject or patient by processing a tumor sample obtained from the subject into multiple tumor fragments;
(e) adding the first population of TILs into a closed system;
(f) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (e) to step (f) occurs without opening the system;
(g) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (f) to step (g) occurs without opening the system;
(h) harvesting therapeutic population of TILs obtained from step (d), wherein the transition from step (d) to step (e) occurs without opening the system; and
(i) transferring the harvested TIL population from step (e) to an infusion bag, wherein the transfer from step (e) to (f) occurs without opening the system;
(j) cryopreserving the infusion bag comprising the harvested TIL population from step (f) using a cryopreservation process; and
(k) administering a therapeutically effective dosage of the third population of TILs from the infusion bag in step (g) to the subject or patient.

In some embodiments, the invention provides a method of treating non-small cell lung carcinoma (NSCLC) by administering a population of tumor infiltrating lymphocytes (TILs) to a patient in need thereof, wherein the method comprises:
(a) testing the patient's tumor for PD-L1 expression and tumor proportion score (TPS) of PD-L1,
(b) testing the patient for the absence of one or more driver mutations, wherein the driver mutation is selected from the group consisting of an EGFR mutation, an EGFR insertion, a KRAS mutation, a BRAF mutation, an ALK mutation, a c-ROS mutation (ROS1 mutation), a ROS1 fusion, a RET mutation, or a RET fusion,
(c) determining that the patient has a TPS score for PD-L1 of about 1% to about 49% and determining that the patient also has no driver mutations,
(d) obtaining and/or receiving a first population of TILs from a tumor resected from the subject or patient by processing a tumor sample obtained from the subject into multiple tumor fragments;
(e) adding the first population of TILs into a closed system;
(f) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (e) to step (f) occurs without opening the system;
(g) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (f) to step (g) occurs without opening the system;
(h) harvesting therapeutic population of TILs obtained from step (d), wherein the transition from step (d) to step (e) occurs without opening the system; and
(i) transferring the harvested TIL population from step (e) to an infusion bag, wherein the transfer from step (e) to (f) occurs without opening the system;
(j) cryopreserving the infusion bag comprising the harvested TIL population from step (f) using a cryopreservation process; and
(k) administering a therapeutically effective dosage of the third population of TILs from the infusion bag in step (g) to the subject or patient.

In some embodiments, the invention provides a method of treating non-small cell lung carcinoma (NSCLC) by administering a population of tumor infiltrating lymphocytes (TILs) to a patient in need thereof, wherein the method comprises:
(a) testing the patient's tumor for PD-L1 expression and tumor proportion score (TPS) of PD-L1,
(b) testing the patient for the absence of one or more driver mutations, wherein the driver mutation is selected from the group consisting of an EGFR mutation, an EGFR insertion, a KRAS mutation, a BRAF mutation, an ALK mutation, a c-ROS mutation (ROS1 mutation), a ROS1 fusion, a RET mutation, or a RET fusion,
(c) determining that the patient has a TPS score for PD-L1 of less than about 1% and determining that the patient also has no driver mutations,
(d) obtaining and/or receiving a first population of TILs from a tumor resected from the subject or patient by processing a tumor sample obtained from the subject into multiple tumor fragments;
(e) adding the first population of TILs into a closed system;
(f) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (e) to step (f) occurs without opening the system;
(g) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (f) to step (g) occurs without opening the system;
(h) harvesting therapeutic population of TILs obtained from step (d), wherein the transition from step (d) to step (e) occurs without opening the system; and
(i) transferring the harvested TIL population from step (e) to an infusion bag, wherein the transfer from step (e) to (f) occurs without opening the system;
(j) cryopreserving the infusion bag comprising the harvested TIL population from step (f) using a cryopreservation process; and
(k) administering a therapeutically effective dosage of the third population of TILs from the infusion bag in step (g) to the subject or patient.

In other embodiments, the invention provides a method for treating a subject with cancer comprising administering to the subject a therapeutically effective dosage of the therapeutic TIL population described herein.

In other embodiments, the invention provides a method for treating a subject with cancer comprising administering to the subject a therapeutically effective dosage of the TIL composition described herein.

In other embodiments, the invention provides the method for treating a subject with cancer described herein modified such that prior to administering the therapeutically effective dosage of the therapeutic TIL population and the TIL composition described herein, respectively, a non-myeloablative lymphodepletion regimen has been administered to the subject.

In other embodiments, the invention provides the method for treating a subject with cancer described herein modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In other embodiments, the invention provides the method for treating a subject with cancer described herein modified to further comprise the step of treating the subject with a high-dose IL-2 regimen starting on the day after administration of the TIL cells to the subject.

In other embodiments, the invention provides the method for treating a subject with cancer described herein modified such that the high-dose IL-2 regimen comprises 600,000 or 720,000 IU/kg administered as a 15-minute bolus intravenous infusion every eight hours until tolerance.

In other embodiments, the invention provides the method for treating a subject with cancer described herein modified such that the cancer is a solid tumor.

In other embodiments, the invention provides the method for treating a subject with cancer described herein modified such that the cancer is melanoma, ovarian cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In other embodiments, the invention provides the method for treating a subject with cancer described herein modified such that the cancer is melanoma, HNSCC, cervical cancers, NSCLC, glioblastoma (including GBM), and gastrointestinal cancer.

In other embodiments, the invention provides the method for treating a subject with cancer described herein modified such that the cancer is melanoma.

In other embodiments, the invention provides the method for treating a subject with cancer described herein modified such that the cancer is HNSCC.

In other embodiments, the invention provides the method for treating a subject with cancer described herein modified such that the cancer is a cervical cancer.

In other embodiments, the invention provides the method for treating a subject with cancer described herein modified such that the cancer is NSCLC.

In other embodiments, the invention provides the method for treating a subject with cancer described herein modified such that the cancer is glioblastoma (including GBM).

In other embodiments, the invention provides a method for treating a subject with cancer described herein modified such that the cancer is gastrointestinal cancer.

In other embodiments, the invention provides a method for treating a subject with cancer described herein modified such that the cancer is a hypermutated cancer.

In other embodiments, the invention provides a method for treating a subject with cancer described herein modified such that the cancer is a pediatric hypermutated cancer.

In other embodiments, the invention provides a therapeutic TIL population described herein for use in a method for treating a subject with cancer comprising administering to the subject a therapeutically effective dosage of the therapeutic TIL population.

In other embodiments, the invention provides a TIL composition described herein for use in a method for treating a subject with cancer comprising administering to the subject a therapeutically effective dosage of the TIL composition.

In other embodiments, the invention provides a therapeutic TIL population described herein or the TIL composition described herein modified such that prior to administering to the subject the therapeutically effective dosage of the therapeutic TIL population described herein or the TIL composition described herein, a non-myeloablative lymphodepletion regimen has been administered to the subject.

In other embodiments, the invention provides a therapeutic TIL population or the TIL composition described herein modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In other embodiments, the invention provides a therapeutic TIL population or a TIL composition described herein modified to further comprise the step of treating patient with a high-dose IL-2 regimen starting on the day after administration of the TIL cells to the patient.

In other embodiments, the invention provides a therapeutic TIL population or a TIL composition described herein modified such that the high-dose IL-2 regimen comprises 600,000 or 720,000 IU/kg administered as a 15-minute bolus intravenous infusion every eight hours until tolerance.

In other embodiments, the invention provides a therapeutic TIL population or a TIL composition described herein modified such that the cancer is a solid tumor.

In other embodiments, the invention provides a therapeutic TIL population or a TIL composition described herein modified such that the cancer is melanoma, ovarian cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In other embodiments, the invention provides a therapeutic TIL population or a TIL composition described herein modified such that the cancer is melanoma, HNSCC, cervical cancers, NSCLC, glioblastoma (including GBM), and gastrointestinal cancer.

In other embodiments, the invention provides a therapeutic TIL population or a TIL composition described herein modified such that the cancer is melanoma.

In other embodiments, the invention provides a therapeutic TIL population or a TIL composition described herein modified such that the cancer is HNSCC.

In other embodiments, the invention provides a therapeutic TIL population or a TIL composition described herein modified such that the cancer is cervical cancer.

In other embodiments, the invention provides a therapeutic TIL population or a TIL composition described herein modified such that the cancer is NSCLC.

In other embodiments, the invention provides a therapeutic TIL population or a TIL composition described herein modified such that the cancer is glioblastoma.

In other embodiments, the invention provides a therapeutic TIL population or a TIL composition described herein modified such that the cancer is gastrointestinal cancer.

In other embodiments, the invention provides a therapeutic TIL population or a TIL composition described herein modified such that the cancer is a hypermutated cancer.

In other embodiments, the invention provides a therapeutic TIL population or a TIL composition described herein modified such that the cancer is a pediatric hypermutated cancer.

In other embodiments, the invention provides the use of a therapeutic TIL population described herein in a method of treating cancer in a subject comprising administering to the subject a therapeutically effective dosage of the therapeutic TIL population.

In other embodiments, the invention provides the use of a TIL composition described in any of the preceding paragraphs in a method of treating cancer in a subject comprising administering to the subject a therapeutically effective dosage of the TIL composition.

In other embodiments, the invention provides the use of a therapeutic TIL population described herein or a TIL composition described herein in a method of treating cancer in a patient comprising administering to the patient a non-myeloablative lymphodepletion regimen and then administering to the subject the therapeutically effective dosage of the therapeutic TIL population described in any of the preceding paragraphs or the therapeutically effective dosage of the TIL composition described herein.

1. Combinations with PD-1 and PD-L1 Inhibitors

In some embodiments, the TIL therapy provided to patients with cancer may include treatment with therapeutic populations of TILs alone or may include a combination treatment including TILs and one or more PD-1 and/or PD-L1 inhibitors.

Programmed death 1 (PD-1) is a 288-amino acid transmembrane immunocheckpoint receptor protein expressed by T cells, B cells, natural killer (NK) T cells, activated monocytes, and dendritic cells. PD-1, which is also known as CD279, belongs to the CD28 family, and in humans is encoded by the Pdcd1 gene on chromosome 2. PD-1 consists of one immunoglobulin (Ig) superfamily domain, a transmembrane region, and an intracellular domain containing an immunoreceptor tyrosine-based inhibitory motif (ITIM) and an immunoreceptor tyrosine-based switch motif (ITSM). PD-1 and its ligands (PD-L1 and PD-L2) are known to play a key role in immune tolerance, as described in Keir, et al., Annu. Rev. Immunol. 2008, 26, 677-704. PD-1 provides inhibitory signals that negatively regulate T cell immune responses. PD-L1 (also known as B7-H1 or CD274) and PD-L2 (also known as B7-DC or CD273) are expressed on tumor cells and stromal cells, which may be encountered by activated T cells expressing PD-1, leading to immunosuppression of the T cells. PD-L1 is a 290 amino acid transmembrane protein encoded by the Cd274 gene on human chromosome 9. Blocking the interaction between PD-1 and its ligands PD-L1 and PD-L2 by use of a PD-1 inhibitor, a PD-L1 inhibitor, and/or a PD-L2 inhibitor can overcome immune resistance, as demonstrated in recent clinical studies, such as that described in Topalian, et al., N. Eng. J Med. 2012, 366, 2443-54. PD-L1 is expressed on many tumor cell lines, while PD-L2 is expressed is expressed mostly on dendritic cells and a few tumor lines. In addition to T cells (which inducibly express PD-1 after activation), PD-1 is also expressed on B cells, natural killer cells, macrophages, activated monocytes, and dendritic cells.

In some embodiments, TILs and a PD-1 inhibitor are administered as a combination therapy or co-therapy for the treatment of NSCLC.

In some embodiments, the NSCLC has undergone no prior therapy. In some embodiments, a PD-1 inhibitor is administered as a front-line therapy or initial therapy. In some embodiments, a PD-1 inhibitor is administered as a front-line therapy or initial therapy in combination with the TILs as described herein.

In some embodiments, the PD-1 inhibitor may be any PD-1 inhibitor or PD-1 blocker known in the art. In particular, it is one of the PD-1 inhibitors or blockers described in more detail in the following paragraphs. The terms “inhibitor,” “antagonist,” and “blocker” are used interchangeably herein in reference to PD-1 inhibitors. For avoidance of doubt, references herein to a PD-1 inhibitor that is an antibody may refer to a compound or antigen-binding fragments, variants, conjugates, or biosimilars thereof. For avoidance of doubt, references herein to a PD-1 inhibitor may also refer to a small molecule compound or a pharmaceutically acceptable salt, ester, solvate, hydrate, cocrystal, or prodrug thereof.

In some embodiments, the PD-1 inhibitor is an antibody (i.e., an anti-PD-1 antibody), a fragment thereof, including Fab fragments, or a single-chain variable fragment (scFv) thereof. In some embodiments the PD-1 inhibitor is a polyclonal antibody. In some embodiments, the PD-1 inhibitor is a monoclonal antibody. In some embodiments, the PD-1 inhibitor competes for binding with PD-1, and/or binds to an epitope on PD-1. In some embodiments, the antibody competes for binding with PD-1, and/or binds to an epitope on PD-1.

In some embodiments, the PD-1 inhibitor is one that binds human PD-1 with a KD of about 100 pM or lower, binds human PD-1 with a KD of about 90 pM or lower, binds human PD-1 with a KD of about 80 pM or lower, binds human PD-1 with a KD of about 70 pM or lower, binds human PD-1 with a KD of about 60 pM or lower, binds human PD-1 with a KD of about 50 pM or lower, binds human PD-1 with a KD of about 40 pM or lower, binds human PD-1 with a KD of about 30 pM or lower, binds human PD-1 with a KD of about 20 pM or lower, binds human PD-1 with a KD of about 10 pM or lower, or binds human PD-1 with a KD of about 1 pM or lower.

In some embodiments, the PD-1 inhibitor is one that binds to human PD-1 with a k_{assoc} of about 7.5×10⁵ l/M·s or faster, binds to human PD-1 with a k_{assoc} of about 7.5×10⁵ l/M·s or faster, binds to human PD-1 with a k_{assoc} of about 8×10⁵ l/M·s or faster, binds to human PD-1 with a k_{assoc} of about 8.5×10⁵ l/M·s or faster, binds to human PD-1 with a k_{assoc} of about 9×10⁵ l/M·s or faster, binds to human PD-1 with a k_{assoc} of about 9.5×10⁵ l/M·s or faster, or binds to human PD-1 with a k_{assoc} of about 1×10⁶ l/M·s or faster.

In some embodiments, the PD-1 inhibitor is one that binds to human PD-1 with a k_{dissoc} of about 2×10⁻⁵ l/s or slower, binds to human PD-1 with a k_{dissoc} of about 2.1×10⁻⁵ l/s or slower, binds to human PD-1 with a k_{dissoc} of about 2.2×10⁻⁵ l/s or slower, binds to human PD-1 with a k_{dissoc} of about 2.3×10⁻⁵ l/s or slower, binds to human PD-1 with a k_{dissoc} of about 2.4×10⁻⁵ l/s or slower, binds to human PD-1 with a k_{dissoc} of about 2.5×10⁻⁵ l/s or slower, binds to human PD-1 with a k_{dissoc} of about 2.6×10⁻⁵ l/s or slower or binds to human PD-1 with a k_{dissoc} of about 2.7×10⁻⁵ l/s or slower, binds to human PD-1 with a k_{dissoc} of about 2.8×10⁻⁵ l/s or slower, binds to human PD-1 with a k_{dissoc} of about 2.9×10⁻⁵ l/s or slower, or binds to human PD-1 with a k_{dissoc} of about 3×10⁻⁵ l/s or slower.

In some embodiments, the PD-1 inhibitor is one that blocks or inhibits binding of human PD-L1 or human PD-L2 to human PD-1 with an IC50 of about 10 nM or lower, blocks or inhibits binding of human PD-L1 or human PD-L2 to human PD-1 with an IC50 of about 9 nM or lower, blocks or inhibits binding of human PD-L1 or human PD-L2 to human PD-1 with an IC50 of about 8 nM or lower, blocks or inhibits binding of human PD-L1 or human PD-L2 to human PD-1 with an IC50 of about 7 nM or lower, blocks or inhibits binding of human PD-L1 or human PD-L2 to human PD-1 with an IC50 of about 6 nM or lower, blocks or inhibits binding of human PD-L1 or human PD-L2 to human PD-1 with an IC50 of about 5 nM or lower, blocks or inhibits binding of human PD-L1 or human PD-L2 to human PD-1 with an IC50 of about 4 nM or lower, blocks or inhibits binding of human PD-L1 or human PD-L2 to human PD-1 with an IC50 of about 3 nM or lower, blocks or inhibits binding of human PD-L1 or human PD-L2 to human PD-1 with an IC50 of about 2 nM or lower, or blocks or inhibits binding of human PD-L1 or human PD-L2 to human PD-1 with an IC50 of about 1 nM or lower.

In some embodiments, the PD-1 inhibitor is nivolumab (commercially available as OPDIVO from Bristol-Myers Squibb Co.), or biosimilars, antigen-binding fragments, conjugates, or variants thereof. Nivolumab is a fully human IgG4 antibody blocking the PD-1 receptor. In some embodiments, the anti-PD-1 antibody is an immunoglobulin G4 kappa, anti-(human CD274) antibody. Nivolumab is assigned Chemical Abstracts Service (CAS) registry number 946414-94-4 and is also known as 5C4, BMS-936558, MDX-1106, and ONO-4538. The preparation and properties of nivolumab are described in U.S. Pat. No. 8,008,449 and International Patent Publication No. WO 2006/121168, the disclosures of which are incorporated by reference herein. The clinical safety and efficacy of nivolumab in various forms of cancer has been described in Wang, et al., Cancer Immunol. Res. 2014, 2, 846-56; Page, et al., Ann. Rev. Med., 2014, 65, 185-202; and Weber, et al., J. Clin. Oncology, 2013, 31, 4311-4318, the disclosures of which are incorporated by reference herein. The amino acid sequences of nivolumab are set forth in Table 26. Nivolumab has intra-heavy chain disulfide linkages at 22-96,140-196, 254-314, 360-418, 22″-96″, 140″-196″, 254″-314″, and 360″-418″; intra-light chain disulfide linkages at 23′-88′, 134′-194′, 23′″-88′″, and 134′″-194′″; inter-heavy-light chain disulfide linkages at 127-214′, 127″-214′″, inter-heavy-heavy chain disulfide linkages at 219-219″ and 222-222″; and N-glycosylation sites (H CH2 84.4) at 290, 290″.

In some embodiments, a PD-1 inhibitor comprises a heavy chain given by SEQ ID NO:158 and a light chain given by SEQ ID NO:159. In some embodiments, a PD-1 inhibitor comprises heavy and light chains having the sequences shown in SEQ ID NO:158 and SEQ ID NO:159, respectively, or antigen binding fragments, Fab fragments, single-chain variable fragments (scFv), variants, or conjugates thereof. In some embodiments, a PD-1 inhibitor comprises heavy and light chains that are each at least 99% identical to the sequences shown in SEQ ID NO:158 and SEQ ID NO:159, respectively. In some embodiments, a PD-1 inhibitor comprises heavy and light chains that are each at least 98% identical to the sequences shown in SEQ ID NO:158 and SEQ ID NO: 159, respectively. In some embodiments, a PD-1 inhibitor comprises heavy and light chains that are each at least 97% identical to the sequences shown in SEQ ID NO:158 and SEQ ID NO:159, respectively. In some embodiments, a PD-1 inhibitor comprises heavy and light chains that are each at least 96% identical to the sequences shown in SEQ ID NO: 158 and SEQ ID NO:159, respectively. In some embodiments, a PD-1 inhibitor comprises heavy and light chains that are each at least 95% identical to the sequences shown in SEQ ID NO:158 and SEQ ID NO:159, respectively.

In some embodiments, the PD-1 inhibitor comprises the heavy and light chain CDRs or variable regions (VRs) of nivolumab. In some embodiments, the PD-1 inhibitor heavy chain variable region (V_{H}) comprises the sequence shown in SEQ ID NO: 160, and the PD-1 inhibitor light chain variable region (V_{L}) comprises the sequence shown in SEQ ID NO:161, or conservative amino acid substitutions thereof. In some embodiments, a PD-1 inhibitor comprises V_{H} and V_{L} regions that are each at least 99% identical to the sequences shown in SEQ ID NO:160 and SEQ ID NO:161, respectively. In some embodiments, a PD-1 inhibitor comprises V_{H} and V_{L} regions that are each at least 98% identical to the sequences shown in SEQ ID NO:160 and SEQ ID NO:161, respectively. In some embodiments, a PD-1 inhibitor comprises V_{H} and V_{L} regions that are each at least 97% identical to the sequences shown in SEQ ID NO:160 and SEQ ID NO:161, respectively. In some embodiments, a PD-1 inhibitor comprises V_{H} and V_{L} regions that are each at least 96% identical to the sequences shown in SEQ ID NO:160 and SEQ ID NO:161, respectively. In some embodiments, a PD-1 inhibitor comprises V_{H} and V_{L} regions that are each at least 95% identical to the sequences shown in SEQ ID NO:160 and SEQ ID NO:161, respectively.

In some embodiments, a PD-1 inhibitor comprises heavy chain CDR1, CDR2 and CDR3 domains having the sequences set forth in SEQ ID NO:162, SEQ ID NO:163, and SEQ ID NO:164, respectively, or conservative amino acid substitutions thereof, and light chain CDR1, CDR2 and CDR3 domains having the sequences set forth in SEQ ID NO:165, SEQ ID NO:166, and SEQ ID NO:167, respectively, or conservative amino acid substitutions thereof. In some embodiments, the antibody competes for binding with, and/or binds to the same epitope on PD-1 as any of the aforementioned antibodies.

In some embodiments, the PD-1 inhibitor is an anti-PD-1 biosimilar monoclonal antibody approved by drug regulatory authorities with reference to nivolumab. In some embodiments, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is nivolumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is nivolumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is nivolumab.

TABLE 26
Amino acid sequences for PD-1 inhibitors related to nivolumab.

| Identifier | Sequence (One-Letter Amino Acid Symbols) | |
|---|---|---|
| SEQ ID NO: 158 | QVQLVESGGG VVQPGRSLRL DCKASGITFS NSGMHWVRQA PGKGLEWVAV IWYDGSKRYY | 60 |
| nivolumab | ADSVKGRFTI SRDNSKNTLF LQMNSLRAED TAVYYCATND DYWGQGTLVT VSSASTKGPS | 120 |
| heavy chain | VFPLAPCSRS TSESTAALGC LVKDYFPEPV TVSWNSGALT SGVHTFPAVL QSSGLYSLSS | 180 |
| | VVTVPSSSLG TKTYTCNVDH KPSNTKVDKR VESKYGPPCP PCPAPEFLGG PSVFLFPPKP | 240 |
| | KDTLMISRTP EVTCVVVDVS QEDPEVQFNW YVDGVEVHNA KTKPREEQFN STYRVVSVLT | 300 |
| | VLHQDWLNGK EYKCKVSNKG LPSSIEKTIS KAKGQPREPQ VYTLPPSQEE MTKNQVSLTC | 360 |
| | LVKGFYPSDI AVEWESNGQP ENNYKTTPPV LDSDGSFFLY SRLTVDKSRW QEGNVFSCSV | 420 |
| | MHEALHNHYT QKSLSLSLGK | 440 |
| SEQ ID NO: 159 | EIVLTQSPAT LSLSPGERAT LSCRASQSVS SYLAWYQQKP GQAPRLLIYD ASNRATGIPA | 60 |
| nivolumab | RFSGSGSGTD FTLTISSLEP EDFAVYYCQQ SSNWPRTFGQ GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 160 | QVQLVESGGG VVQPGRSLRL DCKASGITFS NSGMHWVRQA PGKGLEWVAV IWYDGSKRYY | 60 |
| nivolumab | ADSVKGRFTI SRDNSKNTLF LQMNSLRAED TAVYYCATND DYWGQGTLVT VSS | 113 |
| variable heavy | | |
| chain | | |
| SEQ ID NO: 161 | EIVLTQSPAT LSLSPGERAT LSCRASQSVS SYLAWYQQKP GQAPRLLIYD ASNRATGIPA | 60 |
| nivolumab | RFSGSGSGTD FTLTISSLEP EDFAVYYCQQ SSNWPRTFGQ GTKVEIK | 107 |
| variable light | | |
| chain | | |
| SEQ ID NO: 162 | NSGMH | 5 |
| nivolumab | | |
| heavy chain | | |
| CDR1 | | |
| SEQ ID NO: 163 | VIWYDGSKRY YADSVKG | 17 |
| nivolumab | | |
| heavy chain | | |
| CDR2 | | |
| SEQ ID NO: 164 | NDDY | 4 |
| nivolumab | | |
| heavy chain | | |
| CDR3 | | |
| SEQ ID NO: 165 | RASQSVSSYL A | 11 |
| nivolumab | | |
| light chain | | |
| CDR1 | | |
| SEQ ID NO: 166 | DASNRAT | 7 |
| nivolumab | | |
| light chain | | |
| CDR2 | | |
| SEQ ID NO: 167 | QQSSNWPRT | 9 |
| nivolumab | | |
| light chain | | |
| CDR3 | | |

In some embodiments, the PD-1 inhibitor is nivolumab or a biosimilar thereof, and the nivolumab is administered at a dose of about 0.5 mg/kg to about 10 mg/kg. In some embodiments, the PD-1 inhibitor is nivolumab or a biosimilar thereof, and the nivolumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the PD-1 inhibitor is nivolumab or a biosimilar thereof, and the nivolumab is administered at a dose of about 200 mg to about 500 mg. In some embodiments, the PD-1 inhibitor is nivolumab or a biosimilar thereof, and the nivolumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the PD-1 inhibitor is nivolumab or a biosimilar thereof, and the nivolumab is administered every 2 weeks, every 3 weeks, every 4 weeks, every 5 weeks, or every 6 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat unresectable or metastatic melanoma. In some embodiments, the nivolumab is administered to treat unresectable or metastatic melanoma and is administered at about 240 mg every 2 weeks. In some embodiments, the nivolumab is administered to treat unresectable or metastatic melanoma and is administered at about 480 mg every 4 weeks. In some embodiments, the nivolumab is administered to treat unresectable or metastatic melanoma and is administered at about 1 mg/kg followed by ipilimumab 3 mg/kg on the same day every 3 weeks for 4 doses, then 240 mg every 2 weeks or 480 mg every 4 weeks.

In some embodiments, the nivolumab is administered for the adjuvant treatment of melanoma. In some embodiments, the nivolumab is administered for the adjuvant treatment of melanoma at about 240 mg every 2 weeks. In some embodiments, the nivolumab is administered for the adjuvant treatment of melanoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat metastatic non-small cell lung cancer. In some embodiments, the nivolumab is administered to treat metastatic non-small cell lung cancer at about 3 mg/kg every 2 weeks along with ipilimumab at about 1 mg/kg every 6 weeks. In some embodiments, the nivolumab is administered to treat metastatic non-small cell lung cancer at about 360 mg every 3 weeks with ipilimumab 1 mg/kg every 6 weeks and 2 cycles of platinum-doublet chemotherapy. In some embodiments, the nivolumab is administered to treat metastatic non-small cell lung cancer at about 240 mg every 2 weeks or 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat small cell lung cancer. In some embodiments, the nivolumab is administered to treat small cell lung cancer at about 240 mg every 2 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat malignant pleural mesothelioma at about 360 mg every 3 weeks with ipilimumab 1 mg/kg every 6 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat advanced renal cell carcinoma. In some embodiments, the nivolumab is administered to treat advanced renal cell carcinoma at about 240 mg every 2 weeks. In some embodiments, the nivolumab is administered to treat advanced renal cell carcinoma at about 480 mg every 4 weeks. In some embodiments, the nivolumab is administered to treat advanced renal cell carcinoma at about 3 mg/kg followed by ipilimumab at about 1 mg/kg on the same day every 3 weeks for 4 doses, then 240 mg every 2 weeks. In some embodiments, the nivolumab is administered to treat advanced renal cell carcinoma at about 3 mg/kg followed by ipilimumab at about 1 mg/kg on the same day every 3 weeks for 4 doses, then 240 mg every 2 weeks 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat classical Hodgkin lymphoma. In some embodiments, the nivolumab is administered to treat classical Hodgkin lymphoma at about 240 mg every 2 weeks. In some embodiments, the nivolumab is administered to treat classical Hodgkin lymphoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat Recurrent or metastatic squamous cell carcinoma of the head and neck. In some embodiments, the nivolumab is administered to treat recurrent or metastatic squamous cell carcinoma of the head and neck at about 240 mg every 2 weeks. In some embodiments, the nivolumab is administered to treat recurrent or metastatic squamous cell carcinoma of the head and neck at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat locally advanced or metastatic urothelial carcinoma at about 240 mg every 2 weeks. In some embodiments, the nivolumab is administered to treat locally advanced or metastatic urothelial carcinoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat microsatellite instability-high (MSI-H) or mismatch repair deficient (dMMR) metastatic colorectal cancer. In some embodiments, the nivolumab is administered to treat microsatellite instability-high (MSI-H) or mismatch repair deficient (dMMR) metastatic colorectal cancer in adult and pediatric patients. In some embodiments, the nivolumab is administered to treat microsatellite instability-high (MSI-H) or mismatch repair deficient (dMMR) metastatic colorectal cancer in adult and pediatric patients ≥40 kg at about 240 mg every 2 weeks. In some embodiments, the nivolumab is administered to treat microsatellite instability-high (MSI-H) or mismatch repair deficient (dMMR) metastatic colorectal cancer in adult and pediatric patients ≥40 kg at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat microsatellite instability-high (MSI-H) or mismatch repair deficient (dMMR) metastatic colorectal cancer in pediatric patients <40 kg at about 3 mg/kg every 2 weeks. In some embodiments, the nivolumab is administered to treat microsatellite instability-high (MSI-H) or mismatch repair deficient (dMMR) metastatic colorectal cancer in adult and pediatric patients ≥40 kg at about 3 mg/kg followed by ipilimumab 1 mg/kg on the same day every 3 weeks for 4 doses, then 240 mg every 2 weeks. In some embodiments, the nivolumab is administered to treat microsatellite instability-high (MSI-H) or mismatch repair deficient (dMMR) metastatic colorectal cancer in adult and pediatric patients ≥40 kg at about 3 mg/kg followed by ipilimumab 1 mg/kg on the same day every 3 weeks for 4 doses, then 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat hepatocellular carcinoma. In some embodiments, the nivolumab is administered to treat hepatocellular carcinoma at about 240 mg every 2 weeks. In some embodiments, the nivolumab is administered to treat hepatocellular carcinoma at about 480 mg every 4 weeks. In some embodiments, the nivolumab is administered to treat hepatocellular carcinoma at about 1 mg/kg followed by ipilimumab 3 mg/kg on the same day every 3 weeks for 4 doses, then 240 mg every 2 weeks. In some embodiments, the nivolumab is administered to treat hepatocellular carcinoma at about 1 mg/kg followed by ipilimumab 3 mg/kg on the same day every 3 weeks for 4 doses, then 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In some embodiments, the nivolumab is administered to treat esophageal squamous cell carcinoma. In some embodiments, the nivolumab is administered to treat esophageal squamous cell carcinoma at about 240 mg every 2 weeks. In some embodiments, the nivolumab is administered to treat esophageal squamous cell carcinoma at about 480 mg every 4 weeks. In some embodiments, the nivolumab administration is begun 1, 2, 3, 4, or 5 days post IL-2 administration. In some embodiments, the nivolumab administration is begun 1, 2, or 3 days post IL-2 administration. In some embodiments, the nivolumab can also be administered 1, 2, 3, 4 or 5 weeks pre-resection (i.e., before obtaining a tumor sample from the subject or patient). In some embodiments, the nivolumab can also be administered 1, 2, or 3 weeks pre-resection (i.e., before obtaining a tumor sample from the subject or patient).

In other embodiments, the PD-1 inhibitor comprises pembrolizumab (commercially available as KEYTRUDA from Merck & Co., Inc., Kenilworth, NJ, USA), or antigen-binding fragments, conjugates, or variants thereof. Pembrolizumab is assigned CAS registry number 1374853-91-4 and is also known as lambrolizumab, MK-3475, and SCH-900475. Pembrolizumab has an immunoglobulin G4, anti-(human protein PDCD1 (programmed cell death 1)) (human-Mus musculus monoclonal heavy chain), disulfide with human-Mus musculus monoclonal light chain, dimer structure. The structure of pembrolizumab may also be described as immunoglobulin G4, anti-(human programmed cell death 1); humanized mouse monoclonal [228-L-proline(H10-S>P)]γ4 heavy chain (134-218′)-disulfide with humanized mouse monoclonal K light chain dimer (226-226″:229-229″)-bisdisulfide. The properties, uses, and preparation of pembrolizumab are described in International Patent Publication No. WO 2008/156712 A1, U.S. Pat. No. 8,354,509 and U.S. Patent Application Publication Nos. US 2010/0266617 A1, US 2013/0108651 A1, and US 2013/0109843 A2, the disclosures of which are incorporated herein by reference. The clinical safety and efficacy of pembrolizumab in various forms of cancer is described in Fuerst, Oncology Times, 2014, 36, 35-36; Robert, et al., Lancet, 2014, 384, 1109-17; and Thomas, et al., Exp. Opin. Biol. Ther., 2014, 14, 1061-1064. The amino acid sequences of pembrolizumab are set forth in Table 27. Pembrolizumab includes the following disulfide bridges: 22-96, 22″-96″, 23′-92′, 23′-92′″, 134-218′, 134″-218′″, 138′-198′, 138′-198′″, 147-203, 147″-203″, 226-226″, 229-229″, 261-321, 261″-321″, 367-425, and 367″-425″, and the following glycosylation sites (N): Asn-297 and Asn-297″. Pembrolizumab is an IgG4/kappa isotype with a stabilizing S228P mutation in the Fc region; insertion of this mutation in the IgG4 hinge region prevents the formation of half molecules typically observed for IgG4 antibodies. Pembrolizumab is heterogeneously glycosylated at Asn297 within the Fc domain of each heavy chain, yielding a molecular weight of approximately 149 kDa for the intact antibody. The dominant glycoform of pembrolizumab is the fucosylated agalacto diantennary glycan form (G0F).

In some embodiments, a PD-1 inhibitor comprises a heavy chain given by SEQ ID NO:168 and a light chain given by SEQ ID NO:169. In some embodiments, a PD-1 inhibitor comprises heavy and light chains having the sequences shown in SEQ ID NO:168 and SEQ ID NO:169, respectively, or antigen binding fragments, Fab fragments, single-chain variable fragments (scFv), variants, or conjugates thereof. In some embodiments, a PD-1 inhibitor comprises heavy and light chains that are each at least 99% identical to the sequences shown in SEQ ID NO:168 and SEQ ID NO:169, respectively. In some embodiments, a PD-1 inhibitor comprises heavy and light chains that are each at least 98% identical to the sequences shown in SEQ ID NO:168 and SEQ ID NO: 169, respectively. In some embodiments, a PD-1 inhibitor comprises heavy and light chains that are each at least 97% identical to the sequences shown in SEQ ID NO:168 and SEQ ID NO:169, respectively. In some embodiments, a PD-1 inhibitor comprises heavy and light chains that are each at least 96% identical to the sequences shown in SEQ ID NO: 168 and SEQ ID NO:169, respectively. In some embodiments, a PD-1 inhibitor comprises heavy and light chains that are each at least 95% identical to the sequences shown in SEQ ID NO:168 and SEQ ID NO:169, respectively.

In some embodiments, the PD-1 inhibitor comprises the heavy and light chain CDRs or variable regions (VRs) of pembrolizumab. In some embodiments, the PD-1 inhibitor heavy chain variable region (V_{H}) comprises the sequence shown in SEQ ID NO: 170, and the PD-1 inhibitor light chain variable region (V_{L}) comprises the sequence shown in SEQ ID NO:171, or conservative amino acid substitutions thereof. In some embodiments, a PD-1 inhibitor comprises V_{H} and V_{L} regions that are each at least 99% identical to the sequences shown in SEQ ID NO:170 and SEQ ID NO:171, respectively. In some embodiments, a PD-1 inhibitor comprises V_{H} and V_{L} regions that are each at least 98% identical to the sequences shown in SEQ ID NO:170 and SEQ ID NO:171, respectively. In some embodiments, a PD-1 inhibitor comprises V_{H} and V_{L} regions that are each at least 97% identical to the sequences shown in SEQ ID NO:170 and SEQ ID NO:171, respectively. In some embodiments, a PD-1 inhibitor comprises V_{H} and V_{L} regions that are each at least 96% identical to the sequences shown in SEQ ID NO:170 and SEQ ID NO:171, respectively. In some embodiments, a PD-1 inhibitor comprises V_{H} and V_{L} regions that are each at least 95% identical to the sequences shown in SEQ ID NO:170 and SEQ ID NO:171, respectively.

In some embodiments, a PD-1 inhibitor comprises the heavy chain CDR1, CDR2 and CDR3 domains having the sequences set forth in SEQ ID NO:172, SEQ ID NO:173, and SEQ ID NO:174, respectively, or conservative amino acid substitutions thereof, and light chain CDR1, CDR2 and CDR3 domains having the sequences set forth in SEQ ID NO:175, SEQ ID NO:176, and SEQ ID NO:177, respectively, or conservative amino acid substitutions thereof. In some embodiments, the antibody competes for binding with, and/or binds to the same epitope on PD-1 as any of the aforementioned antibodies.

In some embodiments, the PD-1 inhibitor is an anti-PD-1 biosimilar monoclonal antibody approved by drug regulatory authorities with reference to pembrolizumab. In some embodiments, the biosimilar comprises an anti-PD-1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is pembrolizumab. In some embodiments, the one or more post-translational modifications are selected from one or more of: glycosylation, oxidation, deamidation, and truncation. In some embodiments, the biosimilar is an anti-PD-1 antibody authorized or submitted for authorization, wherein the anti-PD-1 antibody is provided in a formulation which differs from the formulations of a reference medicinal product or reference biological product, wherein the reference medicinal product or reference biological product is pembrolizumab. The anti-PD-1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is pembrolizumab.

TABLE 27
Amino acid sequences for PD-1 inhibitors related to pembrolizumab.

| Identifier | Sequence (One-Letter Amino Acid Symbols) | |
|---|---|---|
| SEQ ID NO: 168 | QVQLVQSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| heavy chain | ASTKGPSVFP LAPCSRSTSE STAALGCLVK DYFPEPVTVS WNSGALTSGV HTFPAVLQSS | 180 |
| | GLYSLSSVVT VPSSSLGTKT YTCNVDHKPS NTKVDKRVES KYGPPCPPCP APEFLGGPSV | 240 |
| | FLFPPKPKDT LMISRTPEVT CVVVDVSQED PEVQFNWYVD GVEVHNAKTK PREEQFNSTY | 300 |
| | RVVSVLTVLH QDWLNGKEYK CKVSNKGLPS SIEKTISKAK GQPREPQVYT LPPSQEEMTK | 360 |
| | NQVSLTCLVK GFYPSDIAVE WESNGQPENN YKTTPPVLDS DGSFFLYSRL TVDKSRWQEG | 420 |
| | NVFSCSVMHE ALHNHYTQKS LSLSLGK | 447 |
| SEQ ID NO: 169 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI KRTVAAPSVE | 120 |
| light chain | IFPPSDEQLK SGTASVVCLL NNFYPREAKV QWKVDNALQS GNSQESVTEQ DSKDSTYSLS | 180 |
| | STLTLSKADY EKHKVYACEV THQGLSSPVT KSFNRGEC | 218 |
| SEQ ID NO: 170 | QVQLVQSGVE VKKPGASVKV SCKASGYTFT NYYMYWVRQA PGQGLEWMGG INPSNGGTNF | 60 |
| pembrolizumab | NEKFKNRVTL TTDSSTTTAY MELKSLQFDD TAVYYCARRD YRFDMGFDYW GQGTTVTVSS | 120 |
| variable heavy | | |
| chain | | |
| SEQ ID NO: 171 | EIVLTQSPAT LSLSPGERAT LSCRASKGVS TSGYSYLHWY QQKPGQAPRL LIYLASYLES | 60 |
| pembrolizumab | GVPARFSGSG SGTDFTLTIS SLEPEDFAVY YCQHSRDLPL TFGGGTKVEI K | 111 |
| variable light | | |
| chain | | |
| SEQ ID NO: 172 | NYYMY | 5 |
| pembrolizumab | | |
| heavy chain | | |
| CDR1 | | |
| SEQ ID NO: 173 | GINPSNGGTN FNEKFK | 16 |
| pembrolizumab | | |
| heavy chain | | |
| CDR2 | | |
| SEQ ID NO: 174 | RDYRFDMGED Y | 11 |
| pembrolizumab | | |
| heavy chain | | |
| CDR3 | | |
| SEQ ID NO: 175 | RASKGVSTSG YSYLH | 15 |
| pembrolizumab | | |
| light chain | | |
| CDR1 | | |
| SEQ ID NO: 176 | LASYLES | 7 |
| pembrolizumab | | |
| light chain | | |
| CDR2 | | |
| SEQ ID NO: 177 | QHSRDLPLT | 9 |
| pembrolizumab | | |
| light chain | | |
| CDR3 | | |

In some embodiments, the PD-1 inhibitor is pembrolizumab or a biosimilar thereof, and the pembrolizumab is administered at a dose of about 0.5 mg/kg to about 10 mg/kg. In some embodiments, the PD-1 inhibitor is pembrolizumab or a biosimilar thereof, and the pembrolizumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the PD-1 inhibitor is pembrolizumab or a biosimilar thereof, wherein the pembrolizumab is administered at a dose of about 200 mg to about 500 mg. In some embodiments, the PD-1 inhibitor is pembrolizumab or a biosimilar thereof, and the nivolumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the PD-1 inhibitor is pembrolizumab or a biosimilar thereof, wherein the pembrolizumab is administered every 2 weeks, every 3 weeks, every 4 weeks, every 5 weeks, or every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat melanoma. In some embodiments, the pembrolizumab is administered to treat melanoma at about 200 mg every 3 weeks. In some embodiments, the pembrolizumab is administered to treat melanoma at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat NSCLC. In some embodiments, the pembrolizumab is administered to treat NSCLC at about 200 mg every 3 weeks. In some embodiments, the pembrolizumab is administered to treat NSCLC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat small cell lung cancer (SCLC). In some embodiments, the pembrolizumab is administered to treat SCLC at about 200 mg every 3 weeks. In some embodiments, the pembrolizumab is administered to treat SCLC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat head and neck squamous cell cancer (HNSCC). In some embodiments, the pembrolizumab is administered to treat HNSCC at about 200 mg every 3 weeks. In some embodiments, the pembrolizumab is administered to treat HNSCCat about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat classical Hodgkin lymphoma (cHL) or primary mediastinal large B-cell lymphoma (PMBCL) at about 200 mg every 3 weeks. In some embodiments, the pembrolizumab is administered to treat classical Hodgkin lymphoma (cHL) or primary mediastinal large B-cell lymphoma (PMBCL) at about 400 mg every 6 weeks for adults. In some embodiments, the pembrolizumab is administered to treat classical Hodgkin lymphoma (cHL) or primary mediastinal large B-cell lymphoma (PMBCL) at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat urothelial carcinoma at about 200 mg every 3 weeks. In some embodiments, the pembrolizumab is administered to treat urothelial carcinoma at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat microsatellite instability-high (MSI-H) or mismatch repair deficient (dMMR) cancer at about 200 mg every 3 weeks. In some embodiments, the pembrolizumab is administered to treat MSI-H or dMMR cancer at about 400 mg every 6 weeks for adults. In some embodiments, the pembrolizumab is administered to treat MSI-H or dMMR cancer at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient). In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat microsatellite instability-high (MSI-H) or mismatch repair deficient colorectal cancer (dMMR CRC at about 200 mg every 3 weeks. In some embodiments, the pembrolizumab is administered to treat MSI-H or dMMR CRC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat gastric cancer at about 200 mg every 3 weeks. In some embodiments, the pembrolizumab is administered to treat gastric cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat Esophageal Cancer at about 200 mg every 3 weeks. In some embodiments, the pembrolizumab is administered to treat Esophageal Cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat cervical cancer at about 200 mg every 3 weeks. In some embodiments, the pembrolizumab is administered to treat cervical cancer at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat hepatocellular carcinoma (HCC) at about 200 mg every 3 weeks. In some embodiments, the pembrolizumab is administered to treat HCC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat Merkel cell carcinoma (MCC) at about 200 mg every 3 weeks for adults. In some embodiments, the pembrolizumab is administered to treat MCC at about 400 mg every 6 weeks for adults. In some embodiments, the pembrolizumab is administered to treat MCC at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient). In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat renal cell carcinoma (RCC) at about 200 mg every 3 weeks. In some embodiments, the pembrolizumab is administered to treat RCC at about 400 mg every 6 weeks with axitinib 5 mg orally twice daily. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat endometrial carcinoma at about 200 mg every 3 weeks. In some embodiments, the pembrolizumab is administered to treat endometrial carcinoma at about 400 mg every 6 weeks with lenvatinib 20 mg orally once daily for tumors that are not MSI-H or dMMR. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat tumor mutational burden-high (TMB-H) Cancer at about 200 mg every 3 weeks for adults. In some embodiments, the pembrolizumab is administered to treat TMB-H Cancer at about 400 mg every 6 weeks for adults. In some embodiments, the pembrolizumab is administered to treat TMB-H Cancer at about 2 mg/kg (up to 200 mg) every 3 weeks for pediatrics. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat cutaneous squamous cell carcinoma (cSCC) at about 200 mg every 3 weeks. In some embodiments, the pembrolizumab is administered to treat cSCC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the pembrolizumab is administered to treat triple-negative breast cancer (TNBC) at about 200 mg every 3 weeks. In some embodiments, the pembrolizumab is administered to treat TNBC at about 400 mg every 6 weeks. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, if the patient or subject is an adult, i.e., treatment of adult indications, and additional dosing regimen of 400 mg every 6 weeks can be employed. In some embodiments, the pembrolizumab administration is begun 1, 2, 3, 4, or 5 days post IL-2 administration. In some embodiments, the pembrolizumab administration is begun 1, 2, or 3 days post IL-2 administration. In some embodiments, the pembrolizumab can also be administered 1, 2, 3, 4 or 5 weeks pre-resection (i.e., before obtaining a tumor sample from the subject or patient). In some embodiments, the pembrolizumab can also be administered 1, 2, or 3 weeks pre-resection (i.e., before obtaining a tumor sample from the subject or patient).

In some embodiments, the PD-1 inhibitor is a commercially-available anti-PD-1 monoclonal antibody, such as anti-m-PD-1 clones J43 (Cat #BE0033-2) and RMP1-14 (Cat #BE0146) (Bio X Cell, Inc., West Lebanon, NH, USA). A number of commercially-available anti-PD-1 antibodies are known to one of ordinary skill in the art.

In some embodiments, the PD-1 inhibitor is an antibody disclosed in U.S. Pat. No. 8,354,509 or U.S. Patent Application Publication Nos. 2010/0266617 A1, 2013/0108651 A1, 2013/0109843 A2, the disclosures of which are incorporated by reference herein. In some embodiments, the PD-1 inhibitor is an anti-PD-1 antibody described in U.S. Pat. Nos. 8,287,856, 8,580,247, and 8,168,757 and U.S. Patent Application Publication Nos. 2009/0028857 Ai, 2010/0285013 Ai, 2013/0022600 Ai, and 2011/0008369 Ai, the teachings of which are hereby incorporated by reference. In other embodiments, the PD-1 inhibitor is an anti-PD-1 antibody disclosed in U.S. Pat. No. 8,735,553 B1, the disclosure of which is incorporated herein by reference. In some embodiments, the PD-1 inhibitor is pidilizumab, also known as CT-011, which is described in U.S. Pat. No. 8,686,119, the disclosure of which is incorporated by reference herein.

In some embodiments, the PD-1 inhibitor may be a small molecule or a peptide, or a peptide derivative, such as those described in U.S. Pat. Nos. 8,907,053; 9,096,642; and 9,044,442 and U.S. Patent Application Publication No. US 2015/0087581; 1,2,4-oxadiazole compounds and derivatives such as those described in U.S. Patent Application Publication No. 2015/0073024; cyclic peptidomimetic compounds and derivatives such as those described in U.S. Patent Application Publication No. US 2015/0073042; cyclic compounds and derivatives such as those described in U.S. Patent Application Publication No. US 2015/0125491; 1,3,4-oxadiazole and 1,3,4-thiadiazole compounds and derivatives such as those described in International Patent Application Publication No. WO 2015/033301; peptide-based compounds and derivatives such as those described in International Patent Application Publication Nos. WO 2015/036927 and WO 2015/04490, or a macrocyclic peptide-based compounds and derivatives such as those described in U.S. Patent Application Publication No. US 2014/0294898; the disclosures of each of which are hereby incorporated by reference in their entireties. In some embodiments, the PD-1 inhibitor is cemiplimab, which is commercially available from Regeneron, Inc.

In some embodiments, TILs and a PD-L1 inhibitor or a PD-L2 inhibitor are administered as a combination therapy or co-therapy for the treatment of NSCLC.

In some embodiments, the NSCLC has undergone no prior therapy. In some embodiments, a PD-L1 inhibitor or a PD-L2 inhibitor is administered as a front-line therapy or initial therapy. In some embodiments, a PD-L1 inhibitor or a PD-L2 inhibitor is administered as a front-line therapy or initial therapy in combination with the TILs as described herein.

In some embodiments, the PD-L1 or PD-L2 inhibitor may be any PD-L1 or PD-L2 inhibitor, antagonist, or blocker known in the art. In particular, it is one of the PD-L1 or PD-L2 inhibitors, antagonist, or blockers described in more detail in the following paragraphs. The terms “inhibitor,” “antagonist,” and “blocker” are used interchangeably herein in reference to PD-L1 and PD-L2 inhibitors. For avoidance of doubt, references herein to a PD-L1 or PD-L2 inhibitor that is an antibody may refer to a compound or antigen-binding fragments, variants, conjugates, or biosimilars thereof. For avoidance of doubt, references herein to a PD-L1 or PD-L2 inhibitor may refer to a compound or a pharmaceutically acceptable salt, ester, solvate, hydrate, cocrystal, or prodrug thereof.

In some embodiments, the compositions, processes and methods described herein include a PD-L1 or PD-L2 inhibitor. In some embodiments, the PD-L1 or PD-L2 inhibitor is a small molecule. In some embodiments, the PD-L1 or PD-L2 inhibitor is an antibody (i.e., an anti-PD-1 antibody), a fragment thereof, including Fab fragments, or a single-chain variable fragment (scFv) thereof. In some embodiments the PD-L1 or PD-L2 inhibitor is a polyclonal antibody. In some embodiments, the PD-L1 or PD-L2 inhibitor is a monoclonal antibody. In some embodiments, the PD-L1 or PD-L2 inhibitor competes for binding with PD-L1 or PD-L2, and/or binds to an epitope on PD-L1 or PD-L2. In some embodiments, the antibody competes for binding with PD-L1 or PD-L2, and/or binds to an epitope on PD-L1 or PD-L2.

In some embodiments, the PD-L1 inhibitors provided herein are selective for PD-L1, in that the compounds bind or interact with PD-L1 at substantially lower concentrations than they bind or interact with other receptors, including the PD-L2 receptor. In certain embodiments, the compounds bind to the PD-L1 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L2 receptor.

In some embodiments, the PD-L2 inhibitors provided herein are selective for PD-L2, in that the compounds bind or interact with PD-L2 at substantially lower concentrations than they bind or interact with other receptors, including the PD-L1 receptor. In certain embodiments, the compounds bind to the PD-L2 receptor at a binding constant that is at least about a 2-fold higher concentration, about a 3-fold higher concentration, about a 5-fold higher concentration, about a 10-fold higher concentration, about a 20-fold higher concentration, about a 30-fold higher concentration, about a 50-fold higher concentration, about a 100-fold higher concentration, about a 200-fold higher concentration, about a 300-fold higher concentration, or about a 500-fold higher concentration than to the PD-L1 receptor.

Without being bound by any theory, it is believed that tumor cells express PD-L1, and that T cells express PD-1. However, PD-L1 expression by tumor cells is not required for efficacy of PD-1 or PD-L1 inhibitors or blockers. In some embodiments, the tumor cells express PD-L1. In other embodiments, the tumor cells do not express PD-L1. In some embodiments, the methods can include a combination of a PD-1 and a PD-L1 antibody, such as those described herein, in combination with a TIL. The administration of a combination of a PD-1 and a PD-L1 antibody and a TIL may be simultaneous or sequential.

In some embodiments, the PD-L1 and/or PD-L2 inhibitor is one that binds human PD-L1 and/or PD-L2 with a KD of about 100 pM or lower, binds human PD-L1 and/or PD-L2 with a KD of about 90 pM or lower, binds human PD-L1 and/or PD-L2 with a KD of about 80 pM or lower, binds human PD-L1 and/or PD-L2 with a KD of about 70 pM or lower, binds human PD-L1 and/or PD-L2 with a KD of about 60 pM or lower, a KD of about 50 pM or lower, binds human PD-L1 and/or PD-L2 with a KD of about 40 pM or lower, or binds human PD-L1 and/or PD-L2 with a KD of about 30 pM or lower,

In some embodiments, the PD-L1 and/or PD-L2 inhibitor is one that binds to human PD-L1 and/or PD-L2 with a k_{assoc} of about 7.5×10⁵ l/M·s or faster, binds to human PD-L1 and/or PD-L2 with a k_{assoc} of about 8×10⁵ l/M·s or faster, binds to human PD-L1 and/or PD-L2 with a k_{assoc} of about 8.5×10⁵ l/M·s or faster, binds to human PD-L1 and/or PD-L2 with a k_{assoc} of about 9×10⁵ l/M·s or faster, binds to human PD-L1 and/or PD-L2 with a k_{assoc} of about 9.5×10⁵ l/M·s and/or faster, or binds to human PD-L1 and/or PD-L2 with a k_{assoc} of about 1×10⁶ l/M·s or faster.

In some embodiments, the PD-L1 and/or PD-L2 inhibitor is one that binds to human PD-L1 or PD-L2 with a k_{dissoc} of about 2×10⁻⁵ l/s or slower, binds to human PD-1 with a k_{dissoc} of about 2.1×10⁻⁵ l/s or slower, binds to human PD-1 with a k_{dissoc} of about 2.2×10⁻⁵ l/s or slower, binds to human PD-1 with a k_{dissoc} of about 2.3×10⁻⁵ l/s or slower, binds to human PD-1 with a k_{dissoc} of about 2.4×10⁻⁵ l/s or slower, binds to human PD-1 with a k_{dissoc} of about 2.5×10⁻⁵ l/s or slower, binds to human PD-1 with a k_{dissoc} of about 2.6×10⁻⁵ l/s or slower, binds to human PD-L1 or PD-L2 with a k_{dissoc} of about 2.7×10⁻⁵ l/s or slower, or binds to human PD-L1 or PD-L2 with a k_{dissoc} of about 3×10⁻⁵ l/s or slower.

In some embodiments, the PD-L1 and/or PD-L2 inhibitor is one that blocks or inhibits binding of human PD-L1 or human PD-L2 to human PD-1 with an IC50 of about 10 nM or lower; blocks or inhibits binding of human PD-L1 or human PD-L2 to human PD-1 with an IC50 of about 9 nM or lower; blocks or inhibits binding of human PD-L1 or human PD-L2 to human PD-1 with an IC50 of about 8 nM or lower; blocks or inhibits binding of human PD-L1 or human PD-L2 to human PD-1 with an IC50 of about 7 nM or lower; blocks or inhibits binding of human PD-L1 or human PD-L2 to human PD-1 with an IC50 of about 6 nM or lower; blocks or inhibits binding of human PD-L1 or human PD-L2 to human PD-1 with an IC50 of about 5 nM or lower; blocks or inhibits binding of human PD-L1 or human PD-L2 to human PD-1 with an IC50 of about 4 nM or lower; blocks or inhibits binding of human PD-L1 or human PD-L2 to human PD-1 with an IC50 of about 3 nM or lower; blocks or inhibits binding of human PD-L1 or human PD-L2 to human PD-1 with an IC50 of about 2 nM or lower; or blocks human PD-1, or blocks binding of human PD-L1 or human PD-L2 to human PD-1 with an IC50 of about 1 nM or lower.

In some embodiments, the PD-L1 inhibitor is durvalumab, also known as MEDI4736 (which is commercially available from Medimmune, LLC, Gaithersburg, Maryland, a subsidiary of AstraZeneca plc.), or antigen-binding fragments, conjugates, or variants thereof. In some embodiments, the PD-L1 inhibitor is an antibody disclosed in U.S. Pat. No. 8,779,108 or U.S. Patent Application Publication No. 2013/0034559, the disclosures of which are incorporated by reference herein. The clinical efficacy of durvalumab has been described in Page, et al., Ann. Rev. Med., 2014, 65, 185-202; Brahmer, et al., J. Clin. Oncol. 2014, 32, 5s (supplement, abstract 8021); and McDermott, et al., Cancer Treatment Rev., 2014, 40, 1056-64. The preparation and properties of durvalumab are described in U.S. Pat. No. 8,779,108, the disclosure of which is incorporated by reference herein. The amino acid sequences of durvalumab are set forth in Table 28. The durvalumab monoclonal antibody includes disulfide linkages at 22-96, 22″-96″, 23′-89′, 23′″-89′″, 135′-195′, 135′-195′″, 148-204, 148″-204″, 215′-224, 215′″-224″, 230-230″, 233-233″, 265-325, 265″-325″, 371-429, and 371″-429′; and N-glycosylation sites at Asn-301 and Asn-301″.

In some embodiments, a PD-L1 inhibitor comprises a heavy chain given by SEQ ID NO:178 and a light chain given by SEQ ID NO:179. In some embodiments, a PD-L1 inhibitor comprises heavy and light chains having the sequences shown in SEQ ID NO:178 and SEQ ID NO:179, respectively, or antigen binding fragments, Fab fragments, single-chain variable fragments (scFv), variants, or conjugates thereof. In some embodiments, a PD-L1 inhibitor comprises heavy and light chains that are each at least 99% identical to the sequences shown in SEQ ID NO:178 and SEQ ID NO: 179, respectively. In some embodiments, a PD-L1 inhibitor comprises heavy and light chains that are each at least 98% identical to the sequences shown in SEQ ID NO:178 and SEQ ID NO:179, respectively. In some embodiments, a PD-L1 inhibitor comprises heavy and light chains that are each at least 97% identical to the sequences shown in SEQ ID NO: 178 and SEQ ID NO:179, respectively. In some embodiments, a PD-L1 inhibitor comprises heavy and light chains that are each at least 96% identical to the sequences shown in SEQ ID NO:178 and SEQ ID NO:179, respectively. In some embodiments, a PD-L1 inhibitor comprises heavy and light chains that are each at least 95% identical to the sequences shown in SEQ ID NO:178 and SEQ ID NO:179, respectively.

In some embodiments, the PD-L1 inhibitor comprises the heavy and light chain CDRs or variable regions (VRs) of durvalumab. In some embodiments, the PD-L1 inhibitor heavy chain variable region (V_{H}) comprises the sequence shown in SEQ ID NO:180, and the PD-L1 inhibitor light chain variable region (V_{L}) comprises the sequence shown in SEQ ID NO:181, or conservative amino acid substitutions thereof. In some embodiments, a PD-L1 inhibitor comprises V_{H} and V_{L} regions that are each at least 99% identical to the sequences shown in SEQ ID NO:180 and SEQ ID NO:181, respectively. In some embodiments, a PD-L1 inhibitor comprises V_{H} and V_{L} regions that are each at least 98% identical to the sequences shown in SEQ ID NO:180 and SEQ ID NO:181, respectively. In some embodiments, a PD-L1 inhibitor comprises V_{H} and V_{L} regions that are each at least 97% identical to the sequences shown in SEQ ID NO:180 and SEQ ID NO:181, respectively. In some embodiments, a PD-L1 inhibitor comprises V_{H} and V_{L} regions that are each at least 96% identical to the sequences shown in SEQ ID NO:180 and SEQ ID NO:181, respectively. In some embodiments, a PD-L1 inhibitor comprises V_{H} and V_{L} regions that are each at least 95% identical to the sequences shown in SEQ ID NO:180 and SEQ ID NO:181, respectively.

In some embodiments, a PD-L1 inhibitor comprises heavy chain CDR1, CDR2 and CDR3 domains having the sequences set forth in SEQ ID NO:182, SEQ ID NO:183, and SEQ ID NO:184, respectively, or conservative amino acid substitutions thereof, and light chain CDR1, CDR2 and CDR3 domains having the sequences set forth in SEQ ID NO:185, SEQ ID NO:186, and SEQ ID NO:187, respectively, or conservative amino acid substitutions thereof. In some embodiments, the antibody competes for binding with, and/or binds to the same epitope on PD-L1 as any of the aforementioned antibodies.

In some embodiments, the PD-L1 inhibitor is an anti-PD-L1 biosimilar monoclonal antibody approved by drug regulatory authorities with reference to durvalumab. In some embodiments, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is durvalumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is durvalumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is durvalumab.

TABLE 28
Amino acid sequences for PD-L1 inhibitors related to durvalumab.

| Identifier | Sequence (One-Letter Amino Acid Symbols) | |
|---|---|---|
| SEQ ID NO: 178 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| heavy chain | SASTKGPSVF PLAPSSKSTS GGTAALGCLV KDYFPEPVTV SWNSGALTSG VHTFPAVLQS | 180 |
| | SGLYSLSSVV TVPSSSLGTQ TYICNVNHKP SNTKVDKRVE PKSCDKTHTC PPCPAPEFEG | 240 |
| | GPSVFLFPPK PKDTLMISRT PEVTCVVVDV SHEDPEVKFN WYVDGVEVHN AKTKPREEQY | 300 |
| | NSTYRVVSVL TVLHQDWLNG KEYKCKVSNK ALPASIEKTI SKAKGQPREP QVYTLPPSRE | 360 |
| | EMTKNQVSLT CLVKGFYPSD IAVEWESNGQ PENNYKTTPP VLDSDGSFFL YSKLTVDKSR | 420 |
| | WQQGNVFSCS VMHEALHNHY TQKSLSLSPG K | 451 |
| SEQ ID NO: 179 | EVQLVESGGG LVQPGGSLRL SCAASGFTFS RYWMSWVRQA PGKGLEWVAN EIVLTQSPGT | 60 |
| durvalumab | LSLSPGERAT LSCRASQRVS SSYLAWYQQK PGQAPRLLIY DASSRATGIP DRFSGSGSGT | 120 |
| light chain | DFTLTISRLE PEDFAVYYCQ QYGSLPWTFG QGTKVEIKRT VAAPSVFIFP PSDEQLKSGT | 180 |
| | ASVVCLINNF YPREAKVQWK VDNALQSGNS QESVTEQDSK DSTYSLSSTL TLSKADYEKH | 240 |
| | KVYACEVTHQ GLSSPVTKSF NRGEC | 265 |
| SEQ ID NO: 180 | EVQLVESGGG LVQPGGSLRL SCAASGFTFS RYWMSWVRQA PGKGLEWVAN IKQDGSEKYY | 60 |
| durvalumab | VDSVKGRFTI SRDNAKNSLY LQMNSLRAED TAVYYCAREG GWFGELAFDY WGQGTLVTVS | 120 |
| variable | S | 121 |
| heavy chain | | |
| SEQ ID NO: 181 | EIVLTQSPGT LSLSPGERAT LSCRASQRVS SSYLAWYQQK PGQAPRLLIY DASSRATGIP | 60 |
| durvalumab | DRFSGSGSGT DFTLTISRLE PEDFAVYYCQ QYGSLPWTFG QGTKVEIK | 108 |
| variable | | |
| light chain | | |
| SEQ ID NO: 182 | RYWMS | 5 |
| durvalumab | | |
| heavy chain | | |
| CDR1 | | |
| SEQ ID NO: 183 | NIKQDGSEKY YVDSVKG | 17 |
| durvalumab | | |
| heavy chain | | |
| CDR2 | | |
| SEQ ID NO: 184 | EGGWFGELAF DY | 12 |
| durvalumab | | |
| heavy chain | | |
| CDR3 | | |
| SEQ ID NO: 185 | RASQRVSSSY LA | 12 |
| durvalumab | | |
| light chain | | |
| CDR1 | | |
| SEQ ID NO: 186 | DASSRAT | 7 |
| durvalumab | | |
| light chain | | |
| CDR2 | | |
| SEQ ID NO: 187 | QQYGSLPWT | 9 |
| durvalumab | | |
| light chain | | |
| CDR3 | | |

In some embodiments, the PD-Li inhibitor is avelumab, also known as MSB0010718C (commercially available from Merck KIGaA/EMD Serono), or antigen-binding fragments, conjugates, or variants thereof. The preparation and properties of avelumab are described in U.S. Patent Application Publication No. US 2014/0341917 A1, the disclosure of which is specifically incorporated by reference herein. The amino acid sequences of avelumab are set forth in Table 29. Avelumab has intra-heavy chain disulfide linkages (C23-C104) at 22-96, 147-203, 264-324, 370-428, 22″-96″, 147″-203″, 264″-324″, and 370″-428″; intra-light chain disulfide linkages (C23-C104) at 22′-90′, 138′-197′, 22″-90′″, and 138′-197′″; intra-heavy-light chain disulfide linkages (h 5-CL 126) at 223-215′ and 223″-215′″; intra-heavy-heavy chain disulfide linkages (h 11, h 14) at 229-229″ and 232-232″; N-glycosylation sites (H CH2 N84.4) at 300, 300″; fucosylated complex bi-antennary CHO-type glycans; and H CHS K2 C-terminal lysine clipping at 450 and 450′.

In some embodiments, a PD-L1 inhibitor comprises a heavy chain given by SEQ ID NO:188 and a light chain given by SEQ ID NO:189. In some embodiments, a PD-L1 inhibitor comprises heavy and light chains having the sequences shown in SEQ ID NO:188 and SEQ ID NO:189, respectively, or antigen binding fragments, Fab fragments, single-chain variable fragments (scFv), variants, or conjugates thereof. In some embodiments, a PD-L1 inhibitor comprises heavy and light chains that are each at least 99% identical to the sequences shown in SEQ ID NO:188 and SEQ ID NO: 189, respectively. In some embodiments, a PD-L1 inhibitor comprises heavy and light chains that are each at least 98% identical to the sequences shown in SEQ ID NO:188 and SEQ ID NO:189, respectively. In some embodiments, a PD-L1 inhibitor comprises heavy and light chains that are each at least 97% identical to the sequences shown in SEQ ID NO: 188 and SEQ ID NO:189, respectively. In some embodiments, a PD-L1 inhibitor comprises heavy and light chains that are each at least 96% identical to the sequences shown in SEQ ID NO:188 and SEQ ID NO:189, respectively. In some embodiments, a PD-L1 inhibitor comprises heavy and light chains that are each at least 95% identical to the sequences shown in SEQ ID NO:188 and SEQ ID NO:189, respectively.

In some embodiments, the PD-L1 inhibitor comprises the heavy and light chain CDRs or variable regions (VRs) of avelumab. In some embodiments, the PD-L1 inhibitor heavy chain variable region (VH) comprises the sequence shown in SEQ ID NO:190, and the PD-L1 inhibitor light chain variable region (VL) comprises the sequence shown in SEQ ID NO:191, or conservative amino acid substitutions thereof. In some embodiments, a PD-L1 inhibitor comprises V_{H} and V_{L} regions that are each at least 99% identical to the sequences shown in SEQ ID NO:190 and SEQ ID NO:191, respectively. In some embodiments, a PD-L1 inhibitor comprises V_{H} and V_{L} regions that are each at least 98% identical to the sequences shown in SEQ ID NO:190 and SEQ ID NO: 191, respectively. In some embodiments, a PD-L1 inhibitor comprises V_{H} and V_{L} regions that are each at least 97% identical to the sequences shown in SEQ ID NO:190 and SEQ ID NO:191, respectively. In some embodiments, a PD-L1 inhibitor comprises V_{H} and V_{L} regions that are each at least 96% identical to the sequences shown in SEQ ID NO: 190 and SEQ ID NO:191, respectively. In some embodiments, a PD-L1 inhibitor comprises V_{H} and V_{L} regions that are each at least 95% identical to the sequences shown in SEQ ID NO:190 and SEQ ID NO:191, respectively.

In some embodiments, a PD-L1 inhibitor comprises heavy chain CDR1, CDR2 and CDR3 domains having the sequences set forth in SEQ ID NO:192, SEQ ID NO:193, and SEQ ID NO:194, respectively, or conservative amino acid substitutions thereof, and light chain CDR1, CDR2 and CDR3 domains having the sequences set forth in SEQ ID NO:195, SEQ ID NO:196, and SEQ ID NO:197, respectively, or conservative amino acid substitutions thereof. In some embodiments, the antibody competes for binding with, and/or binds to the same epitope on PD-L1 as any of the aforementioned antibodies.

In some embodiments, the PD-L1 inhibitor is an anti-PD-L1 biosimilar monoclonal antibody approved by drug regulatory authorities with reference to avelumab. In some embodiments, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is avelumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is avelumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is avelumab.

TABLE 29
Amino acid sequences for PD-L1 inhibitors related to avelumab.

| Identifier | Sequence (One-Letter Amino Acid Symbols) | |
|---|---|---|
| SEQ ID NO: 188 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| heavy chain | ASTKGPSVFP LAPSSKSTSG GTAALGCLVK DYFPEPVTVS WNSGALTSGV HTFPAVLQSS | 180 |
| | GLYSLSSVVT VPSSSLGTQT YICNVNHKPS NTKVDKKVEP KSCDKTHTCP PCPAPELLGG | 240 |
| | PSVFLFPPKP KDTLMISRTP EVTCVVVDVS HEDPEVKFNW YVDGVEVHNA KTKPREEQYN | 300 |
| | STYRVVSVLT VLHQDWINGK EYKCKVSNKA LPAPIEKTIS KAKGQPREPQ VYTLPPSRDE | 360 |
| | LTKNQVSLTC LVKGFYPSDI AVEWESNGQP ENNYKTTPPV LDSDGSFFLY SKLTVDKSRW | 420 |
| | QQGNVFSCSV MHEALHNHYT QKSLSLSPGK | 450 |
| SEQ ID NO: 189 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL GQPKANPTVT | 120 |
| light chain | LFPPSSEELQ ANKATLVCLI SDFYPGAVTV AWKADGSPVK AGVETTKPSK QSNNKYAASS | 180 |
| | YLSLTPEQWK SHRSYSCQVT HEGSTVEKTV APTECS | 216 |
| SEQ ID NO: 190 | EVQLLESGGG LVQPGGSLRL SCAASGFTFS SYIMMWVRQA PGKGLEWVSS IYPSGGITFY | 60 |
| avelumab | ADTVKGRFTI SRDNSKNTLY LQMNSLRAED TAVYYCARIK LGTVTTVDYW GQGTLVTVSS | 120 |
| variable | | |
| heavy chain | | |
| SEQ ID NO: 191 | QSALTQPASV SGSPGQSITI SCTGTSSDVG GYNYVSWYQQ HPGKAPKLMI YDVSNRPSGV | 60 |
| avelumab | SNRFSGSKSG NTASLTISGL QAEDEADYYC SSYTSSSTRV FGTGTKVTVL | 110 |
| variable | | |
| light chain | | |
| SEQ ID NO: 192 | SYIMM | 5 |
| avelumab | | |
| heavy chain | | |
| CDR1 | | |
| SEQ ID NO: 193 | SIYPSGGITF YADTVKG | 17 |
| avelumab | | |
| heavy chain | | |
| CDR2 | | |
| SEQ ID NO: 194 | IKLGTVTTVD Y | 11 |
| avelumab | | |
| heavy chain | | |
| CDR3 | | |
| SEQ ID NO: 195 | TGTSSDVGGY NYVS | 14 |
| avelumab | | |
| light chain | | |
| CDR1 | | |
| SEQ ID NO: 196 | DVSNRPS | 7 |
| avelumab | | |
| light chain | | |
| CDR2 | | |
| SEQ ID NO: 197 | SSYTSSSTRV | 10 |
| avelumab | | |
| light chain | | |
| CDR3 | | |

In some embodiments, the PD-Li inhibitor is atezolizumab, also known as MPDL3280A or RG7446 (commercially available as TECENTRIQ from Genentech, Inc., a subsidiary of Roche Holding AG, Basel, Switzerland), or antigen-binding fragments, conjugates, or variants thereof. In some embodiments, the PD-Li inhibitor is an antibody disclosed in U.S. Pat. No. 8,217,149, the disclosure of which is specifically incorporated by reference herein. In some embodiments, the PD-Li inhibitor is an antibody disclosed in U.S. Patent Application Publication Nos. 2010/0203056 A1, 2013/0045200 A1, 2013/0045201 A1, 2013/0045202 A1, or 2014/0065135 A1, the disclosures of which are specifically incorporated by reference herein. The preparation and properties of atezolizumab are described in U.S. Pat. No. 8,217,149, the disclosure of which is incorporated by reference herein. The amino acid sequences of atezolizumab are set forth in Table 30. Atezolizumab has intra-heavy chain disulfide linkages (C23-C104) at 22-96, 145-201, 262-322, 368-426, 22″-96″, 145″-201″, 262″-322″, and 368″-426″; intra-light chain disulfide linkages (C23-C104) at 23′-88′, 134′-194′, 23′″-88′″, and 134′-194′″; intra-heavy-light chain disulfide linkages (h 5-CL 126) at 221-214′ and 221″-214′″; intra-heavy-heavy chain disulfide linkages (h 11, h 14) at 227-227″ and 230-230″; and N-glycosylation sites (H CH2 N84.4>A) at 298 and 298′.

In some embodiments, a PD-L1 inhibitor comprises a heavy chain given by SEQ ID NO:198 and a light chain given by SEQ ID NO:199. In some embodiments, a PD-L1 inhibitor comprises heavy and light chains having the sequences shown in SEQ ID NO:198 and SEQ ID NO:199, respectively, or antigen binding fragments, Fab fragments, single-chain variable fragments (scFv), variants, or conjugates thereof. In some embodiments, a PD-L1 inhibitor comprises heavy and light chains that are each at least 99% identical to the sequences shown in SEQ ID NO:198 and SEQ ID NO: 199, respectively. In some embodiments, a PD-L1 inhibitor comprises heavy and light chains that are each at least 98% identical to the sequences shown in SEQ ID NO:198 and SEQ ID NO:199, respectively. In some embodiments, a PD-L1 inhibitor comprises heavy and light chains that are each at least 97% identical to the sequences shown in SEQ ID NO: 198 and SEQ ID NO:199, respectively. In some embodiments, a PD-L1 inhibitor comprises heavy and light chains that are each at least 96% identical to the sequences shown in SEQ ID NO:198 and SEQ ID NO:199, respectively. In some embodiments, a PD-L1 inhibitor comprises heavy and light chains that are each at least 95% identical to the sequences shown in SEQ ID NO:198 and SEQ ID NO:199, respectively.

In some embodiments, the PD-L1 inhibitor comprises the heavy and light chain CDRs or variable regions (VRs) of atezolizumab. In some embodiments, the PD-L1 inhibitor heavy chain variable region (V_{H}) comprises the sequence shown in SEQ ID NO:200, and the PD-L1 inhibitor light chain variable region (V_{L}) comprises the sequence shown in SEQ ID NO:201, or conservative amino acid substitutions thereof. In some embodiments, a PD-L1 inhibitor comprises V_{H} and V_{L} regions that are each at least 99% identical to the sequences shown in SEQ ID NO:200 and SEQ ID NO:201, respectively. In some embodiments, a PD-L1 inhibitor comprises V_{H} and V_{L} regions that are each at least 98% identical to the sequences shown in SEQ ID NO:200 and SEQ ID NO:201, respectively. In some embodiments, a PD-L1 inhibitor comprises V_{H} and V_{L} regions that are each at least 97% identical to the sequences shown in SEQ ID NO:200 and SEQ ID NO:201, respectively. In some embodiments, a PD-L1 inhibitor comprises V_{H} and V_{L} regions that are each at least 96% identical to the sequences shown in SEQ ID NO:200 and SEQ ID NO:201, respectively. In some embodiments, a PD-L1 inhibitor comprises V_{H} and V_{L} regions that are each at least 95% identical to the sequences shown in SEQ ID NO:200 and SEQ ID NO:201, respectively.

In some embodiments, a PD-L1 inhibitor comprises heavy chain CDR1, CDR2 and CDR3 domains having the sequences set forth in SEQ ID NO:202, SEQ ID NO:203, and SEQ ID NO:204, respectively, or conservative amino acid substitutions thereof, and light chain CDR1, CDR2 and CDR3 domains having the sequences set forth in SEQ ID NO:205, SEQ ID NO:206, and SEQ ID NO:207, respectively, or conservative amino acid substitutions thereof. In some embodiments, the antibody competes for binding with, and/or binds to the same epitope on PD-L1 as any of the aforementioned antibodies.

In some embodiments, the anti-PD-L1 antibody is an anti-PD-L1 biosimilar monoclonal antibody approved by drug regulatory authorities with reference to atezolizumab. In some embodiments, the biosimilar comprises an anti-PD-L1 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is atezolizumab. In some embodiments, the one or more post-translational modifications are selected from one or more of: glycosylation, oxidation, deamidation, and truncation. In some embodiments, the biosimilar is an anti-PD-L1 antibody authorized or submitted for authorization, wherein the anti-PD-L1 antibody is provided in a formulation which differs from the formulations of a reference medicinal product or reference biological product, wherein the reference medicinal product or reference biological product is atezolizumab. The anti-PD-L1 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is atezolizumab.

TABLE 30
Amino acid sequences for PD-L1 inhibitors related to atezolizumab.

| Identifier | Sequence (One-Letter Amino Acid Symbols) | |
|---|---|---|
| SEQ ID NO: 198 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKKVEPKS CDKTHTCPPC PAPELLGGPS | 240 |
| | VFLFPPKPKD TLMISRTPEV TCVVVDVSHE DPEVKFNWYV DGVEVHNAKT KPREEQYAST | 300 |
| | YRVVSVLTVL HQDWLNGKEY KCKVSNKALP APIEKTISKA KGQPREPQVY TLPPSREEMT | 360 |
| | KNQVSLTCLV KGFYPSDIAV EWESNGQPEN NYKTTPPVLD SDGSFFLYSK LTVDKSRWQQ | 420 |
| | GNVFSCSVMH EALHNHYTQK SLSLSPGK | 448 |
| SEQ ID NO: 199 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 200 | EVQLVESGGG LVQPGGSLRL SCAASGFTFS DSWIHWVRQA PGKGLEWVAW ISPYGGSTYY | 60 |
| atezolizumab | ADSVKGRFTI SADTSKNTAY LQMNSLRAED TAVYYCARRH WPGGFDYWGQ GTLVTVSA | 118 |
| variable | | |
| heavy chain | | |
| SEQ ID NO: 201 | DIQMTQSPSS LSASVGDRVT ITCRASQDVS TAVAWYQQKP GKAPKLLIYS ASFLYSGVPS | 60 |
| atezolizumab | RFSGSGSGTD FTLTISSLQP EDFATYYCQQ YLYHPATFGQ GTKVEIKR | 108 |
| variable | | |
| light chain | | |
| SEQ ID NO: 202 | GFTFSDSWIH | 10 |
| atezolizumab | | |
| heavy chain | | |
| CDR1 | | |
| SEQ ID NO: 203 | AWISPYGGST YYADSVKG | 18 |
| atezolizumab | | |
| heavy chain | | |
| CDR2 | | |
| SEQ ID NO: 204 | RHWPGGFDY | 9 |
| atezolizumab | | |
| heavy chain | | |
| CDR3 | | |
| SEQ ID NO: 205 | RASQDVSTAV A | 11 |
| atezolizumab | | |
| light chain | | |
| CDR1 | | |
| SEQ ID NO: 206 | SASFLYS | 7 |
| atezolizumab | | |
| light chain | | |
| CDR2 | | |
| SEQ ID NO: 207 | QQYLYHPAT | 9 |
| atezolizumab | | |
| light chain | | |
| CDR3 | | |

In some embodiments, PD-Li inhibitors include those antibodies described in U.S. Patent Application Publication No. US 2014/0341917 A1, the disclosure of which is incorporated by reference herein. In other embodiments, antibodies that compete with any of these antibodies for binding to PD-Li are also included. In some embodiments, the anti-PD-Li antibody is MDX-1105, also known as BMS-935559, which is disclosed in U.S. Pat. No. 7,943,743, the disclosures of which are incorporated by reference herein. In some embodiments, the anti-PD-L1 antibody is selected from the anti-PD-L1 antibodies disclosed in U.S. Pat. No. 7,943,743, which are incorporated by reference herein.

In some embodiments, the PD-L1 inhibitor is a commercially-available monoclonal antibody, such as INVIVOMAB anti-m-PD-L1 clone 10F.9G2 (Catalog #BE0101, Bio X Cell, Inc., West Lebanon, NH, USA). In some embodiments, the anti-PD-L1 antibody is a commercially-available monoclonal antibody, such as AFFYMETRIX EBIOSCIENCE (MIH1). A number of commercially-available anti-PD-L1 antibodies are known to one of ordinary skill in the art.

In some embodiments, the PD-L2 inhibitor is a commercially-available monoclonal antibody, such as BIOLEGEND 24F.10C12 Mouse IgG2a, x isotype (catalog #329602 Biolegend, Inc., San Diego, CA), SIGMA anti-PD-L2 antibody (catalog #SAB3500395, Sigma-Aldrich Co., St. Louis, MO), or other commercially-available anti-PD-L2 antibodies known to one of ordinary skill in the art.

2. Combinations with CTLA-4 Inhibitors

In some embodiments, the TIL therapy provided to patients with cancer may include treatment with therapeutic populations of TILs alone or may include a combination treatment including TILs and one or more CTLA-4 inhibitors.

Cytotoxic T lymphocyte antigen 4 (CTLA-4) is a member of the immunoglobulin superfamily and is expressed on the surface of helper T cells. CTLA-4 is a negative regulator of CD28-dependent T cell activation and acts as a checkpoint for adaptive immune responses. Similar to the T cell costimulatory protein CD28, the CTLA-4 binding antigen presents CD80 and CD86 on the cells. CTLA-4 delivers a suppressor signal to T cells, while CD28 delivers a stimulus signal. Human antibodies against human CTLA-4 have been described as immunostimulatory modulators in many disease conditions, such as treating or preventing viral and bacterial infections and for treating cancer (WO 01/14424 and WO 00/37504). A number of fully human anti-human CTLA-4 monoclonal antibodies (mAbs) have been studied in clinical trials for the treatment of various types of solid tumors, including, but not limited to, ipilimumab (MDX-010) and tremelimumab (CP-675,206).

In some embodiments, a CTLA-4 inhibitor may be any CTLA-4 inhibitor or CTLA-4 blocker known in the art. In particular, it is one of the CTLA-4 inhibitors or blockers described in more detail in the following paragraphs. The terms “inhibitor,” “antagonist,” and “blocker” are used interchangeably herein in reference to CTLA-4 inhibitors. For avoidance of doubt, references herein to a CTLA-4 inhibitor that is an antibody may refer to a compound or antigen-binding fragments, variants, conjugates, or biosimilars thereof. For avoidance of doubt, references herein to a CTLA-4 inhibitor may also refer to a small molecule compound or a pharmaceutically acceptable salt, ester, solvate, hydrate, cocrystal, or prodrug thereof.

Suitable CTLA-4 inhibitors for use in the methods of the invention, include, without limitation, anti-CTLA-4 antibodies, human anti-CTLA-4 antibodies, mouse anti-CTLA-4 antibodies, mammalian anti-CTLA-4 antibodies, humanized anti-CTLA-4 antibodies, monoclonal anti-CTLA-4 antibodies, polyclonal anti-CTLA-4 antibodies, chimeric anti-CTLA-4 antibodies, MDX-010 (ipilimumab), tremelimumab, anti-CD28 antibodies, anti-CTLA-4 adnectins, anti-CTLA-4 domain antibodies, single chain anti-CTLA-4 fragments, heavy chain anti-CTLA-4 fragments, light chain anti-CTLA-4 fragments, inhibitors of CTLA-4 that agonize the co-stimulatory pathway, the antibodies disclosed in PCT Publication No. WO 2001/014424, the antibodies disclosed in PCT Publication No. WO 2004/035607, the antibodies disclosed in U.S. Publication No. 2005/0201994, and the antibodies disclosed in granted European Patent No. EP 1212422 B1, the disclosures of each of which are incorporated herein by reference. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014, the disclosures of each of which are incorporated herein by reference. Other anti-CTLA-4 antibodies that can be used in a method of the present invention include, for example, those disclosed in: WO 98/42752; U.S. Pat. Nos. 6,682,736 and 6,207,156; Hurwitz et al., Proc. Natl. Acad. Sci. USA, 95(17):10067-10071 (1998); Camacho et al., J. Clin. Oncology, 22(145): Abstract No. 2505 (2004) (antibody CP-675206); Mokyr et al., Cancer Res., 58:5301-5304 (1998), and U.S. Pat. Nos. 5,977,318, 6,682,736, 7,109,003, and 7,132,281, the disclosures of each of which are incorporated herein by reference.

Additional CTLA-4 inhibitors include, but are not limited to, the following: any inhibitor that is capable of disrupting the ability of CD28 antigen to bind to its cognate ligand, to inhibit the ability of CTLA-4 to bind to its cognate ligand, to augment T cell responses via the co-stimulatory pathway, to disrupt the ability of B7 to bind to CD28 and/or CTLA-4, to disrupt the ability of B7 to activate the co-stimulatory pathway, to disrupt the ability of CD80 to bind to CD28 and/or CTLA-4, to disrupt the ability of CD80 to activate the co-stimulatory pathway, to disrupt the ability of CD86 to bind to CD28 and/or CTLA-4, to disrupt the ability of CD86 to activate the co-stimulatory pathway, and to disrupt the co-stimulatory pathway, in general from being activated. This necessarily includes small molecule inhibitors of CD28, CD80, CD86, CTLA-4, among other members of the co-stimulatory pathway; antibodies directed to CD28, CD80, CD86, CTLA-4, among other members of the co-stimulatory pathway; antisense molecules directed against CD28, CD80, CD86, CTLA-4, among other members of the co-stimulatory pathway; adnectins directed against CD28, CD80, CD86, CTLA-4, among other members of the co-stimulatory pathway, RNAi inhibitors (both single and double stranded) of CD28, CD80, CD86, CTLA-4, among other members of the co-stimulatory pathway, among other CTLA-4 inhibitors.

In some embodiments a CTLA-4 inhibitor binds to CTLA-4 with a K_{d} of about 10⁻⁶ M or less, 10⁻⁷M or less, 10⁻⁸ M or less, 10⁻⁹ M or less, 10⁻¹⁰ M or less, 10⁻¹¹ M or less, 10⁻¹² M or less, e.g., between 10⁻¹³ M and 10⁻¹⁶ M, or within any range having any two of the afore-mentioned values as endpoints. In some embodiments a CTLA-4 inhibitor binds to CTLA-4 with a Kd of no more than 10-fold that of ipilimumab, when compared using the same assay. In some embodiments a CTLA-4 inhibitor binds to CTLA-4 with a Kd of about the same as, or less (e.g., up to 10-fold lower, or up to 100-fold lower) than that of ipilimumab, when compared using the same assay. In some embodiments, the IC50 values for inhibition by a CTLA-4 inhibitor of CTLA-4 binding to CD80 or CD86 is no more than 10-fold greater than that of ipilimumab-mediated inhibition of CTLA-4 binding to CD80 or CD86, respectively, when compared using the same assay. In some embodiments, the IC50 values for inhibition by a CTLA-4 inhibitor of CTLA-4 binding to CD80 or CD86 is about the same or less (e.g., up to 10-fold lower, or up to 100-fold lower) than that of ipilimumab-mediated inhibition of CTLA-4 binding to CD80 or CD86, respectively, when compared using the same assay.

In some embodiments a CTLA-4 inhibitor is used in an amount sufficient to inhibit expression and/or decrease biological activity of CTLA-4 by at least 20%, 30%, 40%, 50%, 60%, 70%, 80%, 90%, 95%, or 100% relative to a suitable control, e.g., between 50% and 75%, 75% and 90%, or 90% and 100%. In some embodiments a CTLA-4 pathway inhibitor is used in an amount sufficient to decrease the biological activity of CTLA-4 by reducing binding of CTLA-4 to CD80, CD86, or both by at least 20%, 30%, 40%, 50%, 60%, 70%, 80%, 90%, 95%, or 100% relative to a suitable control, e.g., between 50% and 75%, 75% and 90%, or 90% and 100% relative to a suitable control. A suitable control in the context of assessing or quantifying the effect of an agent of interest is typically a comparable biological system (e.g., cells or a subject) that has not been exposed to or treated with the agent of interest, e.g., CTLA-4 pathway inhibitor (or has been exposed to or treated with a negligible amount). In some embodiments a biological system may serve as its own control (e.g., the biological system may be assessed before exposure to or treatment with the agent and compared with the state after exposure or treatment has started or finished. In some embodiments a historical control may be used.

In some embodiments, the CTLA-4 inhibitor is ipilimumab (commercially available as Yervoy from Bristol-Myers Squibb Co.), or biosimilars, antigen-binding fragments, conjugates, or variants thereof. As is known in the art, ipilimumab refers to an anti-CTLA-4 antibody, a fully human IgG 1× antibody derived from a transgenic mouse with human genes encoding heavy and light chains to generate a functional human repertoire. is there. Ipilimumab can also be referred to by its CAS Registry Number 477202-00-9, and in PCT Publication Number WO 01/14424, which is incorporated herein by reference in its entirety for all purposes. It is disclosed as antibody 10DI. Specifically, ipilimumab contains a light chain variable region and a heavy chain variable region (having a light chain variable region comprising SEQ ID NO:211 and having a heavy chain variable region comprising SEQ ID NO:210). A pharmaceutical composition of ipilimumab includes all pharmaceutically acceptable compositions containing ipilimumab and one or more diluents, vehicles, or excipients. An example of a pharmaceutical composition containing ipilimumab is described in International Patent Application Publication No. WO 2007/67959. Ipilimumab can be administered intravenously (IV).

In some embodiments, a CTLA-4 inhibitor comprises a heavy chain given by SEQ ID NO:208 and a light chain given by SEQ ID NO:209. In some embodiments, a CTLA-4 inhibitor comprises heavy and light chains having the sequences shown in SEQ ID NO:208 and SEQ ID NO:209, respectively, or antigen binding fragments, Fab fragments, single-chain variable fragments (scFv), variants, or conjugates thereof. In some embodiments, a CTLA-4 inhibitor comprises heavy and light chains that are each at least 99% identical to the sequences shown in SEQ ID NO:208 and SEQ ID NO:209, respectively. In some embodiments, a CTLA-4 inhibitor comprises heavy and light chains that are each at least 98% identical to the sequences shown in SEQ ID NO:208 and SEQ ID NO:209, respectively. In some embodiments, a CTLA-4 inhibitor comprises heavy and light chains that are each at least 97% identical to the sequences shown in SEQ ID NO:208 and SEQ ID NO:209, respectively. In some embodiments, a CTLA-4 inhibitor comprises heavy and light chains that are each at least 96% identical to the sequences shown in SEQ ID NO:208 and SEQ ID NO:209, respectively. In some embodiments, a CTLA-4 inhibitor comprises heavy and light chains that are each at least 95% identical to the sequences shown in SEQ ID NO:208 and SEQ ID NO:209, respectively.

In some embodiments, the CTLA-4 inhibitor comprises the heavy and light chain CDRs or variable regions (VRs) of ipilimumab. In some embodiments, the CTLA-4 inhibitor heavy chain variable region (V_{H}) comprises the sequence shown in SEQ ID NO:210, and the CTLA-4 inhibitor light chain variable region (V_{L}) comprises the sequence shown in SEQ ID NO:211, or conservative amino acid substitutions thereof. In some embodiments, a CTLA-4 inhibitor comprises V_{H} and V_{L} regions that are each at least 99% identical to the sequences shown in SEQ ID NO:210 and SEQ ID NO:211, respectively. In some embodiments, a CTLA-4 inhibitor comprises V_{H} and V_{L} regions that are each at least 98% identical to the sequences shown in SEQ ID NO:210 and SEQ ID NO:211, respectively. In some embodiments, a CTLA-4 inhibitor comprises V_{H} and V_{L} regions that are each at least 97% identical to the sequences shown in SEQ ID NO:210 and SEQ ID NO:211, respectively. In some embodiments, a CTLA-4 inhibitor comprises V_{H} and V_{L} regions that are each at least 96% identical to the sequences shown in SEQ ID NO:210 and SEQ ID NO:211, respectively. In some embodiments, a CTLA-4 inhibitor comprises V_{H} and V_{L} regions that are each at least 95% identical to the sequences shown in SEQ ID NO:210 and SEQ ID NO:211, respectively.

In some embodiments, a CTLA-4 inhibitor comprises the heavy chain CDR1, CDR2 and CDR3 domains having the sequences set forth in SEQ ID NO:212, SEQ ID NO:213, and SEQ ID NO:214, respectively, or conservative amino acid substitutions thereof, and light chain CDR1, CDR2 and CDR3 domains having the sequences set forth in SEQ ID NO:215, SEQ ID NO:216, and SEQ ID NO:217, respectively, or conservative amino acid substitutions thereof. In some embodiments, the antibody competes for binding with, and/or binds to the same epitope on CTLA-4 as any of the aforementioned antibodies.

In some embodiments, the CTLA-4 inhibitor is a CTLA-4 biosimilar monoclonal antibody approved by drug regulatory authorities with reference to ipilimumab. In some embodiments, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is ipilimumab. In some embodiments, the one or more post-translational modifications are selected from one or more of: glycosylation, oxidation, deamidation, and truncation. The amino acid sequences of ipilimumab are set forth in Table 31. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is ipilimumab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is ipilimumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is ipilimumab.

TABLE 31
Amino acid sequences for ipilimumab.

| Identifier | Sequence (One-Letter Amino Acid Symbols) | |
|---|---|---|
| SEQ ID NO: 208 | QVQLVESGGG VVQPGRSLRL SCAASGFTFS SYTMHWVRQA PGKGLEWVTF ISYDGNNKYY | 60 |
| ipilimumab | ADSVKGRFTI SRDNSKNTLY LQMNSLRAED TAIYYCARTG WLGPFDYWGQ GTLVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKRVEPKS CDKTH | 225 |
| SEQ ID NO: 209 | EIVLTQSPGT LSLSPGERAT LSCRASQSVG SSYLAWYQQK PGQAPRLLIY GAFSRATGIP | 60 |
| ipilimumab | DRFSGSGSGT DFTLTISRLE PEDFAVYYCQ QYGSSPWTFG QGTKVEIKRT VAAPSVFIFP | 120 |
| light chain | PSDEQLKSGT ASVVCLLNNF YPREAKVQWK VDNALQSGNS QESVTEQDSK DSTYSLSSTL | 180 |
| | TLSKADYEKH KVYACEVTHQ GLSSPVTKSF NRGEC | 215 |
| SEQ ID NO: 210 | QVQLVESGGG VVQPGRSLRL SCAASGFTFS SYTMHWVRQA PGKGLEWVTF ISYDGNNKYY | 60 |
| ipilimumab | ADSVKGRFTI SRDNSKNTLY LQMNSLRAED TAIYYCARTG WLGPFDYWGQ GTLVTVSS | 118 |
| variable heavy | | |
| chain | | |
| SEQ ID NO: 211 | EIVLTQSPGT LSLSPGERAT LSCRASQSVG SSYLAWYQQK PGQAPRLLIY GAFSRATGIP | 60 |
| ipilimumab | DRFSGSGSGT DFTLTISRLE PEDFAVYYCQ QYGSSPWTFG QGTKVEIK | 108 |
| variable light | | |
| chain | | |
| SEQ ID NO: 212 | GFTFSSYT | 8 |
| ipilimumab | | |
| heavy chain | | |
| CDR1 | | |
| SEQ ID NO: 213 | TFISYDGNNK | 10 |
| ipilimumab | | |
| heavy chain | | |
| CDR2 | | |
| SEQ ID NO: 214 | ARTGWLGPFD Y | 11 |
| ipilimumab | | |
| heavy chain | | |
| CDR3 | | |
| SEQ ID NO: 215 | QSVGSSY | 7 |
| ipilimumab | | |
| light chain | | |
| CDR1 | | |
| SEQ ID NO: 216 | GAF | 3 |
| ipilimumab | | |
| light chain | | |
| CDR2 | | |
| SEQ ID NO: 217 | QQYGSSPWT | 9 |
| ipilimumab | | |
| light chain | | |
| CDR3 | | |

In some embodiments, the CTLA-4 inhibitor is ipilimumab or a biosimilar thereof, and the ipilimumab is administered at a dose of about 0.5 mg/kg to about 10 mg/kg. In some embodiments, the CTLA-4 inhibitor is ipilimumab or a biosimilar thereof, and the ipilimumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the CTLA-4 inhibitor is ipilimumab or a biosimilar thereof, and the ipilimumab is administered at a dose of about 200 mg to about 500 mg. In some embodiments, the CTLA-4 inhibitor is ipilimumab or a biosimilar thereof, and the ipilimumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the CTLA-4 inhibitor is ipilimumab or a biosimilar thereof, and the ipilimumab is administered every 2 weeks, every 3 weeks, every 4 weeks, every 5 weeks, or every 6 weeks. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat unresectable or metastatic melanoma. In some embodiments, the ipilimumab is administered to treat Unresectable or Metastatic Melanoma at about mg/kg every 3 weeks for a maximum of 4 doses. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered for the adjuvant treatment of melanoma. In some embodiments, the ipilimumab is administered to for the adjuvant treatment of melanoma at about 10 mg/kg every 3 weeks for 4 doses, followed by 10 mg/kg every 12 weeks for up to 3 years. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat advanced renal cell carcinoma. In some embodiments, the ipilimumab is administered to treat advanced renal cell carcinoma at about 1 mg/kg immediately following nivolumab 3 mg/kg on the same day, every 3 weeks for 4 doses. In some embodiments, after completing 4 doses of the combination, nivolumab can be administered as a single agent according to standard dosing regimens for advanced renal cell carcinoma and/or renal cell carcinoma. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat microsatellite instability-high (MSI-H) or mismatch repair deficient (dMMR) metastatic colorectal cancer. In some embodiments, the ipilimumab is administered to treat microsatellite instability-high (MSI-H) or mismatch repair deficient (dMMR) metastatic colorectal cancer at about 1 mg/kg intravenously over 30 minutes immediately following nivolumab 3 mg/kg intravenously over 30 minutes on the same day, every 3 weeks for 4 doses. In some embodiments, after completing 4 doses of the combination, administer nivolumab as a single agent as recommended according to standard dosing regimens for microsatellite instability-high (MSI-H) or mismatch repair deficient (dMMR) metastatic colorectal cancer. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat hepatocellular carcinoma. In some embodiments, the ipilimumab is administered to treat hepatocellular carcinoma at about 3 mg/kg intravenously over 30 minutes immediately following nivolumab 1 mg/kg intravenously over 30 minutes on the same day, every 3 weeks for 4 doses. In some embodiments, after completion 4 doses of the combination, administer nivolumab as a single agent according to standard dosing regimens for hepatocellular carcinoma. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat metastatic non-small cell lung cancer. In some embodiments, the ipilimumab is administered to treat metastatic non-small cell lung cancer at about 1 mg/kg every 6 weeks with nivolumab 3 mg/kg every 2 weeks. In some embodiments, the ipilimumab is administered to treat metastatic non-small cell lung cancer at about 1 mg/kg every 6 weeks with nivolumab 360 mg every 3 weeks and 2 cycles of platinum-doublet chemotherapy. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

In some embodiments, the ipilimumab is administered to treat malignant pleural mesothelioma. In some embodiments, the ipilimumab is administered to treat malignant pleural mesothelioma at about 1 mg/kg every 6 weeks with nivolumab 360 mg every 3 weeks. In some embodiments, the ipilimumab administration is begun 1, 2, 3, 4, or 5 weeks pre-resection (i.e., prior to obtaining the tumor sample from the subject or patient). In some embodiments, the ipilimumab administration is begun 1, 2, or 3 weeks pre-resection (i.e., prior to obtaining the tumor sample from the subject or patient).

Tremelimumab (also known as CP-675,206) is a fully human IgG2 monoclonal antibody and has the CAS number 745013-59-6. Tremelimumab is disclosed as antibody 11.2.1 in U.S. Pat. No. 6,682,736 (incorporated herein by reference). The amino acid sequences of the heavy chain and light chain of tremelimumab are set forth in SEQ ID NOs:218 and 219, respectively. Tremelimumab has been investigated in clinical trials for the treatment of various tumors, including melanoma and breast cancer; in which Tremelimumab was administered intravenously either as single dose or multiple doses every 4 or 12 weeks at the dose range of 0.01 and 15 mg/kg. In the regimens provided by the present invention, tremelimumab is administered locally, particularly intradermally or subcutaneously. The effective amount of tremelimumab administered intradermally or subcutaneously is typically in the range of 5-200 mg/dose per person. In some embodiments, the effective amount of tremelimumab is in the range of 10-150 mg/dose per person per dose. In some particular embodiments, the effective amount of tremelimumab is about 10, 25, 37.5, 40, 50, 75, 100, 125, 150, 175, or 200 mg/dose per person.

In some embodiments, a CTLA-4 inhibitor comprises a heavy chain given by SEQ ID NO:218 and a light chain given by SEQ ID NO:219. In some embodiments, a CTLA-4 inhibitor comprises heavy and light chains having the sequences shown in SEQ ID NO:218 and SEQ ID NO:219, respectively, or antigen binding fragments, Fab fragments, single-chain variable fragments (scFv), variants, or conjugates thereof. In some embodiments, a CTLA-4 inhibitor comprises heavy and light chains that are each at least 99% identical to the sequences shown in SEQ ID NO:218 and SEQ ID NO:219, respectively. In some embodiments, a CTLA-4 inhibitor comprises heavy and light chains that are each at least 98% identical to the sequences shown in SEQ ID NO:218 and SEQ ID NO:219, respectively. In some embodiments, a CTLA-4 inhibitor comprises heavy and light chains that are each at least 97% identical to the sequences shown in SEQ ID NO:218 and SEQ ID NO:219, respectively. In some embodiments, a CTLA-4 inhibitor comprises heavy and light chains that are each at least 96% identical to the sequences shown in SEQ ID NO:218 and SEQ ID NO:219, respectively. In some embodiments, a CTLA-4 inhibitor comprises heavy and light chains that are each at least 95% identical to the sequences shown in SEQ ID NO:218 and SEQ ID NO:219, respectively.

In some embodiments, the CTLA-4 inhibitor comprises the heavy and light chain CDRs or variable regions (VRs) of tremelimumab. In some embodiments, the CTLA-4 inhibitor heavy chain variable region (V_{H}) comprises the sequence shown in SEQ ID NO:220, and the CTLA-4 inhibitor light chain variable region (V_{L}) comprises the sequence shown in SEQ ID NO:221, or conservative amino acid substitutions thereof. In some embodiments, a CTLA-4 inhibitor comprises V_{H} and V_{L} regions that are each at least 99% identical to the sequences shown in SEQ ID NO:220 and SEQ ID NO:221, respectively. In some embodiments, a CTLA-4 inhibitor comprises V_{H} and V_{L} regions that are each at least 98% identical to the sequences shown in SEQ ID NO:220 and SEQ ID NO:221, respectively. In some embodiments, a CTLA-4 inhibitor comprises V_{H} and V_{L} regions that are each at least 97% identical to the sequences shown in SEQ ID NO:220 and SEQ ID NO:221, respectively. In some embodiments, a CTLA-4 inhibitor comprises V_{H} and V_{L} regions that are each at least 96% identical to the sequences shown in SEQ ID NO:220 and SEQ ID NO:221, respectively. In some embodiments, a CTLA-4 inhibitor comprises V_{H} and V_{L} regions that are each at least 95% identical to the sequences shown in SEQ ID NO:220 and SEQ ID NO:221, respectively. In some embodiments, a CTLA-4 inhibitor comprises the heavy chain CDR1, CDR2 and CDR3 domains having the sequences set forth in SEQ ID NO:222, SEQ ID NO:223, and SEQ ID NO:224, respectively, or conservative amino acid substitutions thereof, and light chain CDR1, CDR2 and CDR3 domains having the sequences set forth in SEQ ID NO:225, SEQ ID NO:226, and SEQ ID NO:227, respectively, or conservative amino acid substitutions thereof. In some embodiments, the antibody competes for binding with, and/or binds to the same epitope on CTLA-4 as any of the aforementioned antibodies.

In some embodiments, the CTLA-4 inhibitor is an anti-CTLA-4 biosimilar monoclonal antibody approved by drug regulatory authorities with reference to tremelimumab. In some embodiments, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is tremelimumab. In some embodiments, the one or more post-translational modifications are selected from one or more of: glycosylation, oxidation, deamidation, and truncation. The amino acid sequences of tremelimumab are set forth in Table 32. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is tremelimumab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tremelimumab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is tremelimumab.

TABLE 32
Amino acid sequences for tremelimumab.

| Identifier | Sequence (One-Letter Amino Acid Symbols) | |
|---|---|---|
| SEQ ID NO: 218 | QVQLVESGGG VVQPGRSLRL SCAASGFTFS SYGMHWVRQA PGKGLEWVAV IWYDGSNKYY | 60 |
| tremelimumab | ADSVKGRFTI SRDNSKNTLY LQMNSLRAED TAVYYCARDP RGATLYYYYY GMDVWGQGTT | 120 |
| heavy chain | VTVSSASTKG PSVFPLAPCS RSTSESTAAL GCLVKDYFPE PVTVSWNSGA LTSGVHTFPA | 180 |
| | VLQSSGLYSL SSVVTVPSSN FGTQTYTCNV DHKPSNTKVD KTVERKCCVE CPPCPAPPVA | 240 |
| | GPSVFLFPPK PKDTLMISRT PEVTCVVVDV SHEDPEVQFN WYVDGVEVHN AKTKPREEQF | 300 |
| | NSTFRVVSVL TVVHQDWING KEYKCKVSNK GLPAPIEKTI SKTKGQPREP QVYTLPPSRE | 360 |
| | EMTKNQVSLT CLVKGFYPSD IAVEWESNGQ PENNYKTTPP MLDSDGSFFL YSKLTVDKSR | 420 |
| | WQQGNVFSCS VMHEALHNHY TQKSLSLSPG K | 451 |
| SEQ ID NO: 219 | DIQMTQSPSS LSASVGDRVT ITCRASQSIN SYLDWYQQKP GKAPKLLIYA ASSLQSGVPS | 60 |
| tremelimumab | RFSGSGSGTD FTLTISSLQP EDFATYYCQQ YYSTPFTFGP GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 220 | GVVQPGRSLR LSCAASGFTF SSYGMHWVRQ APGKGLEWVA VIWYDGSNKY YADSVKGRFT | 60 |
| tremelimumab | ISRDNSKNTL YLQMNSLRAE DTAVYYCARD PRGATLYYYY YGMDVWGQGT TVTVSSASTK | 120 |
| variable heavy | GPSVFPLAPC SRSTSESTAA LGCLVKDYFP EPVTVSWNSG ALTSGVH | 167 |
| chain | | |
| SEQ ID NO: 221 | PSSLSASVGD RVTITCRASQ SINSYLDWYQ QKPGKAPKLL IYAASSLQSG VPSRFSGSGS | 60 |
| tremelimumab | GTDFTLTISS LQPEDFATYY CQQYYSTPFT FGPGTKVEIK RTVAAPSVFI FPPSDEQLKS | 120 |
| variable light | GTASVVCLLN NFYPREAKV | 139 |
| chain | | |
| SEQ ID NO: 222 | GFTFSSYGMH | 10 |
| tremelimumab | | |
| heavy chain | | |
| CDR1 | | |
| SEQ ID NO: 223 | VIWYDGSNKY YADSV | 15 |
| tremelimumab | | |
| heavy chain | | |
| CDR2 | | |
| SEQ ID NO: 224 | DPRGATLYYY YYGMDV | 16 |
| tremelimumab | | |
| heavy chain | | |
| CDR3 | | |
| SEQ ID NO: 225 | RASQSINSYL D | 11 |
| tremelimumab | | |
| light chain | | |
| CDR1 | | |
| SEQ ID NO: 226 | AASSLQS | 7 |
| tremelimumab | | |
| light chain | | |
| CDR2 | | |
| SEQ ID NO: 227 | QQYYSTPFT | 9 |
| tremelimumab | | |
| light chain | | |
| CDR3 | | |

In some embodiments, the CTLA-4 inhibitor is tremelimumab or a biosimilar thereof, and the tremelimumab is administered at a dose of about 0.5 mg/kg to about 10 mg/kg. In some embodiments, the CTLA-4 inhibitor is tremelimumab or a biosimilar thereof, and the tremelimumab is administered at a dose of about 0.5 mg/kg, about 1 mg/kg, about 1.5 mg/kg, about 2 mg/kg, about 2.5 mg/kg, about 3 mg/kg, about 3.5 mg/kg, about 4 mg/kg, about 4.5 mg/kg, about 5 mg/kg, about 5.5 mg/kg, about 6 mg/kg, about 6.5 mg/kg, about 7 mg/kg, about 7.5 mg/kg, about 8 mg/kg, about 8.5 mg/kg, about 9 mg/kg, about 9.5 mg/kg, or about 10 mg/kg. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In some embodiments, the CTLA-4 inhibitor is tremelimumab or a biosimilar thereof, and the tremelimumab is administered at a dose of about 200 mg to about 500 mg. In some embodiments, the CTLA-4 inhibitor is tremelimumab or a biosimilar thereof, and the tremelimumab is administered at a dose of about 200 mg, about 220 mg, about 240 mg, about 260 mg, about 280 mg, about 300 mg, about 320 mg, about 340 mg, about 360 mg, about 380 mg, about 400 mg, about 420 mg, about 440 mg, about 460 mg, about 480 mg, or about 500 mg. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In some embodiments, the CTLA-4 inhibitor is tremelimumab or a biosimilar thereof, and the tremelimumab is administered every 2 weeks, every 3 weeks, every 4 weeks, every 5 weeks, or every 6 weeks. In some embodiments, the tremelimumab administration is begun 1, 2, 3, 4, or 5 weeks pre-resection (i.e., prior to obtaining the tumor sample from the subject or patient). In some embodiments, the tremelimumab administration is begun 1, 2, or 3 weeks pre-resection (i.e., prior to obtaining the tumor sample from the subject or patient).

In some embodiments, the CTLA-4 inhibitor is zalifrelimab from Agenus, or biosimilars, antigen-binding fragments, conjugates, or variants thereof. Zalifrelimab is a fully human monoclonal antibody. Zalifrelimab is assigned Chemical Abstracts Service (CAS) registry number 2148321-69-9 and is also known as also known as AGEN1884. The preparation and properties of zalifrelimab are described in U.S. Pat. No. 10,144,779 and US Patent Application Publication No. US2020/0024350 A1, the disclosures of which are incorporated by reference herein.

In some embodiments, a CTLA-4 inhibitor comprises a heavy chain given by SEQ ID NO:228 and a light chain given by SEQ ID NO:229. In some embodiments, a CTLA-4 inhibitor comprises heavy and light chains having the sequences shown in SEQ ID NO:228 and SEQ ID NO:229, respectively, or antigen binding fragments, Fab fragments, single-chain variable fragments (scFv), variants, or conjugates thereof. In some embodiments, a CTLA-4 inhibitor comprises heavy and light chains that are each at least 99% identical to the sequences shown in SEQ ID NO:228 and SEQ ID NO:229, respectively. In some embodiments, a CTLA-4 inhibitor comprises heavy and light chains that are each at least 98% identical to the sequences shown in SEQ ID NO:228 and SEQ ID NO:229, respectively. In some embodiments, a CTLA-4 inhibitor comprises heavy and light chains that are each at least 97% identical to the sequences shown in SEQ ID NO:228 and SEQ ID NO:229, respectively. In some embodiments, a CTLA-4 inhibitor comprises heavy and light chains that are each at least 96% identical to the sequences shown in SEQ ID NO:228 and SEQ ID NO:229, respectively. In some embodiments, a CTLA-4 inhibitor comprises heavy and light chains that are each at least 95% identical to the sequences shown in SEQ ID NO:228 and SEQ ID NO:229, respectively.

In some embodiments, the CTLA-4 inhibitor comprises the heavy and light chain CDRs or variable regions (VRs) of zalifrelimab. In some embodiments, the CTLA-4 inhibitor heavy chain variable region (V_{H}) comprises the sequence shown in SEQ ID NO:230, and the CTLA-4 inhibitor light chain variable region (V_{L}) comprises the sequence shown in SEQ ID NO:231, or conservative amino acid substitutions thereof. In some embodiments, a CTLA-4 inhibitor comprises V_{H} and V_{L} regions that are each at least 99% identical to the sequences shown in SEQ ID NO:230 and SEQ ID NO:231, respectively. In some embodiments, a CTLA-4 inhibitor comprises V_{H} and V_{L} regions that are each at least 98% identical to the sequences shown in SEQ ID NO:230 and SEQ ID NO:231, respectively. In some embodiments, a CTLA-4 inhibitor comprises V_{H} and V_{L} regions that are each at least 97% identical to the sequences shown in SEQ ID NO:230 and SEQ ID NO:231, respectively. In some embodiments, a CTLA-4 inhibitor comprises V_{H} and V_{L} regions that are each at least 96% identical to the sequences shown in SEQ ID NO:230 and SEQ ID NO:231, respectively. In some embodiments, a CTLA-4 inhibitor comprises V_{H} and V_{L} regions that are each at least 95% identical to the sequences shown in SEQ ID NO:230 and SEQ ID NO:231, respectively.

In some embodiments, a CTLA-4 inhibitor comprises the heavy chain CDR1, CDR2 and CDR3 domains having the sequences set forth in SEQ ID NO:231, SEQ ID NO:233, and SEQ ID NO:234, respectively, or conservative amino acid substitutions thereof, and light chain CDR1, CDR2 and CDR3 domains having the sequences set forth in SEQ ID NO:235, SEQ ID NO:236, and SEQ ID NO:237, respectively, or conservative amino acid substitutions thereof. In some embodiments, the antibody competes for binding with, and/or binds to the same epitope on CTLA-4 as any of the aforementioned antibodies.

In some embodiments, the CTLA-4 inhibitor is a CTLA-4 biosimilar monoclonal antibody approved by drug regulatory authorities with reference to zalifrelimab. In some embodiments, the biosimilar comprises an anti-CTLA-4 antibody comprising an amino acid sequence which has at least 97% sequence identity, e.g., 97%, 98%, 99% or 100% sequence identity, to the amino acid sequence of a reference medicinal product or reference biological product and which comprises one or more post-translational modifications as compared to the reference medicinal product or reference biological product, wherein the reference medicinal product or reference biological product is zalifrelimab. In some embodiments, the one or more post-translational modifications are selected from one or more of: glycosylation, oxidation, deamidation, and truncation. The amino acid sequences of zalifrelimab are set forth in Table 33. In some embodiments, the biosimilar is an anti-CTLA-4 antibody authorized or submitted for authorization, wherein the anti-CTLA-4 antibody is provided in a formulation which differs from the formulations of a reference medicinal product or reference biological product, wherein the reference medicinal product or reference biological product is zalifrelimab. The anti-CTLA-4 antibody may be authorized by a drug regulatory authority such as the U.S. FDA and/or the European Union's EMA. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is zalifrelimab. In some embodiments, the biosimilar is provided as a composition which further comprises one or more excipients, wherein the one or more excipients are the same or different to the excipients comprised in a reference medicinal product or reference biological product, wherein the reference medicinal product or reference biological product is zalifrelimab.

TABLE 33
Amino acid sequences for zalifrelimab.

| Identifier | Sequence (One-Letter Amino Acid Symbols) | |
|---|---|---|
| SEQ ID NO: 228 | EVQLVESGGG LVKPGGSLRL SCAASGFTFS SYSMNWVRQA PGKGLEWVSS ISSSSSYIYY | 60 |
| zalifrelimab | ADSVKGRFTI SRDNAKNSLY LQMNSLRAED TAVYYCARVG LMGPFDIWGQ GTMVTVSSAS | 120 |
| heavy chain | TKGPSVFPLA PSSKSTSGGT AALGCLVKDY FPEPVTVSWN SGALTSGVHT FPAVLQSSGL | 180 |
| | YSLSSVVTVP SSSLGTQTYI CNVNHKPSNT KVDKRVEPKS CDKTHTCPPC PAPELLGGPS | 240 |
| | VFLFPPKPKD TLMISRTPEV TCVVVDVSHE DPEVKFNWYV DGVEVHNAKT KPREEQYNST | 300 |
| | YRVVSVLTVL HQDWLNGKEY KCKVSNKALP APIEKTISKA KGQPREPQVY TLPPSREEMT | 360 |
| | KNQVSLTCLV KGFYPSDIAV EWESNGQPEN NYKTTPPVLD SDGSFFLYSK LTVDKSRWQQ | 420 |
| | GNVFSCSVMH EALHNHYTQK SLSLSPGK | 448 |
| SEQ ID NO: 229 | EIVLTQSPGT LSLSPGERAT LSCRASQSVS RYLGWYQQKP GQAPRLLIYG ASTRATGIPD | 60 |
| zalifrelimab | RFSGSGSGTD FTLTITRLEP EDFAVYYCQQ YGSSPWTFGQ GTKVEIKRTV AAPSVFIFPP | 120 |
| light chain | SDEQLKSGTA SVVCLLNNFY PREAKVQWKV DNALQSGNSQ ESVTEQDSKD STYSLSSTLT | 180 |
| | LSKADYEKHK VYACEVTHQG LSSPVTKSFN RGEC | 214 |
| SEQ ID NO: 230 | EVQLVESGGG LVKPGGSLRL SCAASGFTFS SYSMNWVRQA PGKGLEWVSS ISSSSSYIYY | 60 |
| zalifrelimab | ADSVKGRFTI SRDNAKNSLY LQMNSLRAED TAVYYCARVG LMGPFDIWGQ GTMVTVSS | 118 |
| variable heavy | | |
| chain | | |
| SEQ ID NO: 231 | EIVLTQSPGT LSLSPGERAT LSCRASQSVS RYLGWYQQKP GQAPRLLIYG ASTRATGIPD | 60 |
| zalifrelimab | RFSGSGSGTD FTLTITRLEP EDFAVYYCQQ YGSSPWTFGQ GTKVEIK | 107 |
| variable light | | |
| chain | | |
| SEQ ID NO: 232 | GFTFSSYS | 8 |
| zalifrelimab | | |
| heavy chain | | |
| CDR1 | | |
| SEQ ID NO: 233 | ISSSSSYI | 8 |
| zalifrelimab | | |
| heavy chain | | |
| CDR2 | | |
| SEQ ID NO: 234 | ARVGLMGPFD I | 11 |
| zalifrelimab | | |
| heavy chain | | |
| CDR3 | | |
| SEQ ID NO: 235 | QSVSRY | 6 |
| zalifrelimab | | |
| light chain | | |
| CDR1 | | |
| SEQ ID NO: 236 | GAS | 3 |
| zalifrelimab | | |
| light chain | | |
| CDR2 | | |
| SEQ ID NO: 237 | QQYGSSPWT | 9 |
| zalifrelimab | | |
| light chain | | |
| CDR3 | | |

Examples of additional anti-CTLA-4 antibodies includes, but are not limited to: AGEN1181, BMS-986218, BCD-145, ONC-392, CS1002, REGN4659, and ADG116, which are known to one of ordinary skill in the art.

In some embodiments, the anti-CTLA-4 antibody is an anti-CTLA-4 antibody disclosed in any of the following patent publications: US 2019/0048096 A1; US 2020/0223907; US 2019/0201334; US 2019/0201334; US 2005/0201994; EP 1212422 Bi; WO 2018/204760; WO 2018/204760; WO 2001/014424; WO 2004/035607; WO 2003/086459; WO 2012/120125; WO 2000/037504; WO 2009/100140; WO 2006/09649; WO2005092380; WO 2007/123737; WO 2006/029219; WO 2010/0979597; WO 2006/12168; and WO1997020574, each of which is incorporated herein by reference. Additional CTLA-4 antibodies are described in U.S. Pat. Nos. 5,811,097, 5,855,887, 6,051,227, and 6,984,720; in PCT Publication Nos. WO 01/14424 and WO 00/37504; and in U.S. Publication Nos. 2002/0039581 and 2002/086014; and/or U.S. Pat. Nos. 5,977,318, 6,682,736, 7,109,003, and 7,132,281, each of which is incorporated herein by reference. In some embodiments, the anti-CTLA-4 antibody is, for example, those disclosed in: WO 98/42752; U.S. Pat. Nos. 6,682,736 and 6,207,156; Hurwitz, et al., Proc. Natl. Acad. Sci. USA, 1998, 95, 10067-10071 (1998); Camacho, et al., J Clin. Oncol., 2004, 22, 145 (Abstract No. 2505 (2004) (antibody CP-675206); or Mokyr, et al., Cancer Res., 1998, 58, 5301-5304 (1998), each of which is incorporated herein by reference.

In some embodiments, the CTLA-4 inhibitor is a CTLA-4 ligand as disclosed in WO 1996/040915 (incorporated herein by reference).

In some embodiments, the CTLA-4 inhibitor is a nucleic acid inhibitor of CTLA-4 expression. For example, anti-CTLA-4 RNAi molecules may take the form of the molecules described in PCT Publication Nos. WO 1999/032619 and WO 2001/029058; U.S. Publication Nos. 2003/0051263, 2003/0055020, 2003/0056235, 2004/265839, 2005/0100913, 2006/0024798, 2008/0050342, 2008/0081373, 2008/0248576, and 2008/055443; and/or U.S. Pat. Nos. 6,506,559, 7,282,564, 7,538,095, and 7,560,438 (incorporated herein by reference). In some instances, the anti-CTLA-4 RNAi molecules take the form of double stranded RNAi molecules described in European Patent No. EP 1309726 (incorporated herein by reference). In some instances, the anti-CTLA-4 RNAi molecules take the form of double stranded RNAi molecules described in U.S. Pat. Nos. 7,056,704 and 7,078,196 (incorporated herein by reference). In some embodiments, the CTLA-4 inhibitor is an aptamer described in International Patent Application Publication No. WO 2004/081021 (incorporated herein by reference).

In other embodiments, the anti-CTLA-4 RNAi molecules of the present invention are RNA molecules described in U.S. Pat. Nos. 5,898,031, 6,107,094, 7,432,249, and 7,432,250, and European Application No. EP 0928290 (incorporated herein by reference).

### 3. Lymphodepletion Preconditioning of Patients

In some embodiments, the invention includes a method of treating a cancer with a population of TILs, wherein a patient is pre-treated with non-myeloablative chemotherapy prior to an infusion of TILs according to the present disclosure. In some embodiments, the invention includes a population of TILs for use in the treatment of cancer in a patient which has been pre-treated with non-myeloablative chemotherapy. In some embodiments, the population of TILs is for administration by infusion. In some embodiments, the non-myeloablative chemotherapy is cyclophosphamide 60 mg/kg/d for 2 days (days 27 and 26 prior to TIL infusion) and fludarabine 25 mg/m²/d for 5 days (days 27 to 23 prior to TIL infusion). In some embodiments, after non-myeloablative chemotherapy and TIL infusion (at day 0) according to the present disclosure, the patient receives an intravenous infusion of IL-2 (aldesleukin, commercially available as PROLEUKIN) intravenously at 720,000 IU/kg every 8 hours to physiologic tolerance. In certain embodiments, the population of TILs is for use in treating cancer in combination with IL-2, wherein the IL-2 is administered after the population of TILs.

In some embodiments, the patient receives a reduced intensity non-myeloablative lyphodepletion regimen. In some embodiments, the reduced intensity non-myeloablative lyphodepletion regimen comprises administration of cyclophosphamide at a dose of about 250-750 mg/m²/day. In some embodiments, cyclophosphamide is administered at a dose of about 250 mg/m²/day. In some embodiments, cyclophosphamide is administered at a dose of about 500 mg/m²/day. In some embodiments, cyclophosphamide is administered at a dose of about 750 mg/m²/day. In some embodiments, cyclophosphamide is administered for three or four days. In some embodiments, the administration of cyclophosphamide is followed by administration of fludarabine at a dose of 30 mg/m²/day. In some embodiments, fludarabine is administered for three, four, or five days. In some embodiments, the cyclophosphamide is administered with mesna. In some embodiments, the patient receives no non-myeloablative lyphodepletion regimen.

Experimental findings indicate that lymphodepletion prior to adoptive transfer of tumor-specific T lymphocytes plays a key role in enhancing treatment efficacy by eliminating regulatory T cells and competing elements of the immune system (‘cytokine sinks’). Accordingly, some embodiments of the invention utilize a lymphodepletion step (sometimes also referred to as “immunosuppressive conditioning”) on the patient prior to the introduction of the TILs of the invention.

In general, lymphodepletion is achieved using administration of fludarabine or cyclophosphamide (the active form being referred to as mafosfamide) and combinations thereof. Such methods are described in Gassner, et al., Cancer Immunol. Immunother. 2011, 60, 75-85, Muranski, et al., Nat. Clin. Pract. Oncol., 2006, 3, 668-681, Dudley, et al., J Clin. Oncol. 2008, 26, 5233-5239, and Dudley, et al., J Clin. Oncol. 2005, 23, 2346-2357, all of which are incorporated by reference herein in their entireties.

In some embodiments, the fludarabine is administered at a concentration of 0.5 μg/mL to 10 μg/mL fludarabine. In some embodiments, the fludarabine is administered at a concentration of 1 μg/mL fludarabine. In some embodiments, the fludarabine treatment is administered for 1 day, 2 days, 3 days, 4 days, 5 days, 6 days, or 7 days or more. In some embodiments, the fludarabine is administered at a dosage of 10 mg/kg/day, 15 mg/kg/day, 20 mg/kg/day, 25 mg/kg/day, 30 mg/kg/day, 35 mg/kg/day, 40 mg/kg/day, or 45 mg/kg/day. In some embodiments, the fludarabine treatment is administered for 2-7 days at 35 mg/kg/day. In some embodiments, the fludarabine treatment is administered for 4-5 days at 35 mg/kg/day. In some embodiments, the fludarabine treatment is administered for 4-5 days at 25 mg/kg/day.

In some embodiments, the mafosfamide, the active form of cyclophosphamide, is obtained at a concentration of 0.5 μg/mL to 10 μg/mL by administration of cyclophosphamide. In some embodiments, mafosfamide, the active form of cyclophosphamide, is obtained at a concentration of 1 μg/mL by administration of cyclophosphamide. In some embodiments, the cyclophosphamide treatment is administered for 1 day, 2 days, 3 days, 4 days, 5 days, 6 days, or 7 days or more. In some embodiments, the cyclophosphamide is administered at a dosage of 100 mg/m²/day, 150 mg/m²/day, 175 mg/m²/day, 200 mg/m²/day, 225 mg/m²/day, 250 mg/m²/day, 275 mg/m²/day, or 300 mg/m²/day. In some embodiments, the cyclophosphamide is administered intravenously (i.e., i.v.) In some embodiments, the cyclophosphamide treatment is administered for 2-7 days at 35 mg/kg/day. In some embodiments, the cyclophosphamide treatment is administered for 4-5 days at 250 mg/m²/day i.v. In some embodiments, the cyclophosphamide treatment is administered for 4 days at 250 mg/m²/day i.v.

In some embodiments, lymphodepletion is performed by administering the fludarabine and the cyclophosphamide together to a patient. In some embodiments, fludarabine is administered at 25 mg/m²/day i.v. and cyclophosphamide is administered at 250 mg/m²/day i.v. over 4 days.

In some embodiments, the lymphodepletion is performed by administration of cyclophosphamide at a dose of 60 mg/m²/day for two days followed by administration of fludarabine at a dose of 25 mg/m²/day for five days.

In some embodiments, the lymphodepletion is performed by administration of cyclophosphamide at a dose of 60 mg/m²/day for two days and administration of fludarabine at a dose of 25 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 50 mg/m²/day for two days and administration of fludarabine at a dose of about 25 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 50 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 40 mg/m²/day for two days and administration of fludarabine at a dose of about 20 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of about 40 mg/m²/day for two days and administration of fludarabine at a dose of about 15 mg/m²/day for five days, wherein cyclophosphamide and fludarabine are both administered on the first two days, and wherein the lymphodepletion is performed in five days in total.

In some embodiments, the lymphodepletion is performed by administration of cyclophosphamide at a dose of 60 mg/m²/day and fludarabine at a dose of 25 mg/m²/day for two days followed by administration of fludarabine at a dose of 25 mg/m²/day for three days.

In some embodiments, the cyclophosphamide is administered with mesna. In some embodiments, mesna is administered at 15 mg/kg. In some embodiments where mesna is infused, and if infused continuously, mesna can be infused over approximately 2 hours with cyclophosphamide (on Days −5 and/or −4), then at a rate of 3 mg/kg/hour for the remaining 22 hours over the 24 hours starting concomitantly with each cyclophosphamide dose.

In some embodiments, the lymphodepletion comprises the step of treating the patient with an IL-2 regimen starting on the day after administration of the third population of TILs to the patient.

In some embodiments, the lymphodepletion comprises the step of treating the patient with an IL-2 regimen starting on the same day as administration of the third population of TILs to the patient.

In some embodiments, the lymphodeplete comprises 5 days of preconditioning treatment. In some embodiments, the days are indicated as days −5 through −1, or Day 0 through Day 4. In some embodiments, the regimen comprises cyclophosphamide on days −5 and −4 (i.e., days 0 and 1). In some embodiments, the regimen comprises intravenous cyclophosphamide on days −5 and −4 (i.e., days 0 and 1). In some embodiments, the regimen comprises 60 mg/kg intravenous cyclophosphamide on days −5 and −4 (i.e., days 0 and 1). In some embodiments, the cyclophosphamide is administered with mesna. In some embodiments, the regimen further comprises fludarabine. In some embodiments, the regimen further comprises intravenous fludarabine. In some embodiments, the regimen further comprises 25 mg/m² intravenous fludarabine. In some embodiments, the regimen further comprises 25 mg/m² intravenous fludarabine on days −5 and −1 (i.e., days 0 through 4). In some embodiments, the regimen further comprises 25 mg/m² intravenous fludarabine on days −5 and −1 (i.e., days 0 through 4).

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for five days.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for three days

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for one day.

In some embodiments, the non-myeloablative lymphodepletion regimen comprises the steps of administration of cyclophosphamide at a dose of 60 mg/m²/day for two days followed by administration of fludarabine at a dose of 25 mg/m²/day for three days.

In some embodiments, the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

In some embodiments, the non-myeloablative lymphodepletion regimen is administered according to Table 34.

TABLE 34
Exemplary lymphodepletion and treatment regimen.

| Day | −5 | −4 | −3 | −2 | −1 | 0 | 1 | 2 | 3 | 4 |
|---|---|---|---|---|---|---|---|---|---|---|
| Cyclophosphamide 60 mg/kg | X | X | | | | | | | | |
| Mesna (as needed) | X | X | | | | | | | | |
| Fludarabine 25 mg/m²/day | X | X | X | X | X | | | | | |
| TIL infusion | | | | | | X | | | | |

In some embodiments, the non-myeloablative lymphodepletion regimen is administered according to Table 35.

TABLE 35
Exemplary lymphodepletion and treatment regimen.

| Day | −4 | −3 | −2 | −1 | 0 | 1 | 2 | 3 | 4 |
|---|---|---|---|---|---|---|---|---|---|
| Cyclophosphamide 60 mg/kg | X | X | | | | | | | |
| Mesna (as needed) | X | X | | | | | | | |
| Fludarabine 25 mg/m²/day | X | X | X | X | | | | | |
| TIL infusion | | | | | X | | | | |

In some embodiments, the non-myeloablative lymphodepletion regimen is administered according to Table 36.

TABLE 36
Exemplary lymphodepletion and treatment regimen.

| Day | −3 | −2 | −1 | 0 | 1 | 2 | 3 | 4 |
|---|---|---|---|---|---|---|---|---|
| Cyclophosphamide 60 mg/kg | X | X | | | | | | |
| Mesna (as needed) | X | X | | | | | | |
| Fludarabine 25 mg/m²/day | X | X | X | | | | | |
| TIL infusion | | | | X | | | | |

In some embodiments, the non-myeloablative lymphodepletion regimen is administered according to Table 37.

TABLE 37
Exemplary lymphodepletion and treatment regimen.

| Day | −5 | −4 | −3 | −2 | −1 | 0 | 1 | 2 | 3 | 4 |
|---|---|---|---|---|---|---|---|---|---|---|
| Cyclophosphamide 60 mg/kg | X | X | | | | | | | | |
| Mesna (as needed) | X | X | | | | | | | | |
| Fludarabine 25 mg/m²/day | | | X | X | X | | | | | |
| TIL infusion | | | | | | X | | | | |

In some embodiments, the non-myeloablative lymphodepletion regimen is administered according to Table 38.

TABLE 38
Exemplary lymphodepletion and treatment regimen.

| Day | −5 | −4 | −3 | −2 | −1 | 0 | 1 | 2 | 3 | 4 |
|---|---|---|---|---|---|---|---|---|---|---|
| Cyclophosphamide 300 mg/kg | X | X | | | | | | | | |
| Mesna (as needed) | X | X | | | | | | | | |
| Fludarabine 30 mg/m²/day | X | X | X | X | X | | | | | |
| TIL infusion | | | | | | X | | | | |

In some embodiments, the non-myeloablative lymphodepletion regimen is administered according to Table 39.

TABLE 38
Exemplary lymphodepletion and treatment regimen.

| Day | −4 | −3 | −2 | −1 | 0 | 1 | 2 | 3 | 4 |
|---|---|---|---|---|---|---|---|---|---|
| Cyclophosphamide 300 mg/kg | X | X | | | | | | | |
| Mesna (as needed) | X | X | | | | | | | |
| Fludarabine 30 mg/m²/day | X | X | X | X | | | | | |
| TIL infusion | | | | | X | | | | |

In some embodiments, the non-myeloablative lymphodepletion regimen is administered according to Table 40.

TABLE 40
Exemplary lymphodepletion and treatment regimen.

| Day | −3 | −2 | −1 | 0 | 1 | 2 | 3 | 4 |
|---|---|---|---|---|---|---|---|---|
| Cyclophosphamide 300 mg/kg | X | X | | | | | | |
| Mesna (as needed) | X | X | | | | | | |
| Fludarabine 30 mg/m²/day | X | X | X | | | | | |
| TIL infusion | | | | X | | | | |

In some embodiments, the non-myeloablative lymphodepletion regimen is administered according to Table 41.

TABLE 41
Exemplary lymphodepletion and treatment regimen.

| Day | −5 | −4 | −3 | −2 | −1 | 0 | 1 | 2 | 3 | 4 |
|---|---|---|---|---|---|---|---|---|---|---|
| Cyclophosphamide 300 mg/kg | X | X | | | | | | | | |
| Mesna (as needed) | X | X | | | | | | | | |
| Fludarabine 30 mg/m²/day | | | X | X | X | | | | | |
| TIL infusion | | | | | | X | | | | |

In some embodiments, the TIL infusion used with the foregoing embodiments of myeloablative lymphodepletion regimens may be any TIL composition described herein, as well as the addition of IL-2 regimens and administration of co-therapies (such as PD-1 and PD-L1 inhibitors) as described herein.

In some embodiments, the non-myeloablative lymphodepletion regimen comprises melphalan administered to a total dose of 100 mg/m² over the course of 1, 2, or 3 days prior to the day of TIL infusion. In some embodiments, the non-myeloablative lymphodepletion regimen comprises melphalan administered to a total dose of 200 mg/m² over the course of 1, 2, or 3 days prior to the day of TIL infusion. In some embodiments, the non-myeloablative lymphodepletion regimen comprises melphalan administered to a total dose of 100 mg/m² and fludarabine administered at a dose of 30 mg/m²/day over the course of 1, 2, or 3 days prior to the day of TIL infusion. In some embodiments, the non-myeloablative lymphodepletion regimen comprises melphalan administered to a total dose of 200 mg/m² and fludarabine administered at a dose of 30 mg/m²/day over the course of 1, 2, or 3 days prior to the day of TIL infusion.

In some embodiments, the non-myeloablative lymphodepletion regimen comprises administration of an anti-CD45 antibody. In some embodiments, the non-myeloablative lymphodepletion regimen comprises administration of an anti-CD45 antibody-drug conjugate. In some embodiments, the non-myeloablative lymphodepletion regimen comprises administration of an anti-CD45 antibody-radioisotope conjugate. In some embodiments, the non-myeloablative lymphodepletion regimen comprises administration of apamistamab-¹³¹I. In some embodiments, the non-myeloablative lymphodepletion regimen comprises apamistamab-¹³¹I administered at a dose of 25 mCi, 50 mCi, 75 mCi, 100 mCi, 150 mCi, or 200 mCi between 2 and 9 days prior to TIL infusion. In some embodiments, the non-myeloablative lymphodepletion regimen comprises apamistamab-¹³¹I administered at a dose of 25 mCi to 200 mCi between 2 and 9 days prior to TIL infusion. In some embodiments, the non-myeloablative lymphodepletion regimen comprises apamistamab-¹³¹I administered at a dose of 50 mCi to 150 mCi between 4 and 8 days prior to TIL infusion. In some embodiments, the non-myeloablative lymphodepletion regimen comprises apamistamab-¹³¹I administered at a dose of about 75 mCi about 6 days prior to TIL infusion. In some embodiments, the non-myeloablative lymphodepletion regimen comprises apamistamab-¹³¹I administered at a dose of about 100 mCi about 7 days prior to TIL infusion.

In some embodiments, the TIL infusion used with the foregoing embodiments of myeloablative lymphodepletion regimens may be any TIL composition described herein, including TIL products genetically modified to include one or more immunomodulatory agents attached to its surface, and may also include infusions of MILs and PBLs in place of the TIL infusion, as well as the addition of alternative lymphodepletion regimens including the anti-CD52 antibody alemtuzumab, or variants, fragments, antibody-drug conjugates, or biosimilars thereof.

### 4. IL-2 Regimens

In some embodiments, the IL-2 regimen comprises a high-dose IL-2 regimen, wherein the high-dose IL-2 regimen comprises aldesleukin, or a biosimilar or variant thereof, administered intravenously starting on the day after administering a therapeutically effective portion of the therapeutic population of TILs, wherein the aldesleukin or a biosimilar or variant thereof is administered at a dose of 0.037 mg/kg or 0.044 mg/kg IU/kg (patient body mass) using 15-minute bolus intravenous infusions every eight hours until tolerance, for a maximum of 14 doses. Following 9 days of rest, this schedule may be repeated for another 14 doses, for a maximum of 28 doses in total. In some embodiments, IL-2 is administered in 1, 2, 3, 4, 5, or 6 doses. In some embodiments, IL-2 is administered at a maximum dosage of up to 6 doses.

In some embodiments, the IL-2 regimen comprises a decrescendo IL-2 regimen. Decrescendo IL-2 regimens have been described in O'Day, et al., J. Clin. Oncol. 1999, 17, 2752-61 and Eton, et al., Cancer 2000, 88, 1703-9, the disclosures of which are incorporated herein by reference. In some embodiments, a decrescendo IL-2 regimen comprises 18×10⁶ IU/m² aldesleukin, or a biosimilar or variant thereof, administered intravenously over 6 hours, followed by 18×10⁶ IU/m² administered intravenously over 12 hours, followed by 18×10⁶ IU/m² administered intravenously over 24 hours, followed by 4.5×10⁶ IU/m² administered intravenously over 72 hours. This treatment cycle may be repeated every 28 days for a maximum of four cycles. In some embodiments, a decrescendo IL-2 regimen comprises 18,000,000 IU/m² on day 1, 9,000,000 IU/m² on day 2, and 4,500,000 IU/m² on days 3 and 4.

In some embodiments, a reduced-dose IL-2 regimen comprises a reduce number, e.g., 1, 2, 3, 4, or 5, doses of 600,000 or 720,000 IU/kg of aldesleukin, or a biosimilar or variant thereof, administered as a 15-minute bolus intravenous infusion every eight hours until tolerance. In some embodiments, the patient receives no IL-2 regimen.

In some embodiments, the IL-2 regimen comprises a low-dose IL-2 regimen. Any low-dose IL-2 regimen known in the art may be used, including the low-dose IL-2 regimens described in Dominguez-Villar and Hafler, Nat. Immunology 2000, 19, 665-673; Hartemann, et al., Lancet Diabetes Endocrinol. 2013, 1, 295-305; and Rosenzwaig, et al., Ann. Rheum. Dis. 2019, 78, 209-217, the disclosures of which are incorporated herein by reference. In some embodiments, a low-dose IL-2 regimen comprises 18×10⁶ IU per m² of aldesleukin, or a biosimilar or variant thereof, per 24 hours, administered as a continuous infusion for 5 days, followed by 2-6 days without IL-2 therapy, optionally followed by an additional 5 days of intravenous aldesleukin or a biosimilar or variant thereof, as a continuous infusion of 18×10⁶ IU per m² per 24 hours, optionally followed by 3 weeks without IL-2 therapy, after which additional cycles may be administered.

In some embodiments, IL-2 is administered at a maximum dosage of up to 6 doses. In some embodiments, the high-dose IL-2 regimen is adapted for pediatric use. In some embodiments, a dose of 600,000 international units (IU)/kg of aldesleukin every 8-12 hours for up to a maximum of 6 doses is used. In some embodiments, a dose of 500,000 international units (IU)/kg of aldesleukin every 8-12 hours for up to a maximum of 6 doses is used. In some embodiments, a dose of 400,000 international units (IU)/kg of aldesleukin every 8-12 hours for up to a maximum of 6 doses is used. In some embodiments, a dose of 500,000 international units (IU)/kg of aldesleukin every 8-12 hours for up to a maximum of 6 doses is used. In some embodiments, a dose of 300,000 international units (IU)/kg of aldesleukin every 8-12 hours for up to a maximum of 6 doses is used. In some embodiments, a dose of 200,000 international units (IU)/kg of aldesleukin every 8-12 hours for up to a maximum of 6 doses is used. In some embodiments, a dose of 100,000 international units (IU)/kg of aldesleukin every 8-12 hours for up to a maximum of 6 doses is used.

In some embodiments, the IL-2 regimen comprises administration of pegylated IL-2 every 1, 2, 4, 6, 7, 14 or 21 days at a dose of 0.10 mg/day to 50 mg/day. In some embodiments, the IL-2 regimen comprises administration of bempegaldesleukin, or a fragment, variant, or biosimilar thereof, every 1, 2, 4, 6, 7, 14 or 21 days at a dose of 0.10 mg/day to 50 mg/day.

In some embodiments, the IL-2 regimen comprises administration of THOR-707, or a fragment, variant, or biosimilar thereof, every 1, 2, 4, 6, 7, 14 or 21 days at a dose of 0.10 mg/day to 50 mg/day.

In some embodiments, the IL-2 regimen comprises administration of nemvaleukin alfa, or a fragment, variant, or biosimilar thereof, following administration of TIL. In certain embodiments, the patient the nemvaleukin is administered every 1, 2, 4, 6, 7, 14 or 21 days at a dose of 0.10 mg/day to 50 mg/day.

In some embodiments, the IL-2 regimen comprises administration of an IL-2 fragment engrafted onto an antibody backbone. In some embodiments, the IL-2 regimen comprises administration of an antibody-cytokine engrafted protein that binds the IL-2 low affinity receptor. In some embodiments, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the antibody cytokine engrafted protein preferentially expands T effector cells over regulatory T cells. In some embodiments, the antibody cytokine engrafted protein comprises a heavy chain variable region (V_{H}), comprising complementarity determining regions HCDR1, HCDR2, HCDR3; a light chain variable region (V_{L}), comprising LCDR1, LCDR2, LCDR3; and an IL-2 molecule or a fragment thereof engrafted into a CDR of the V_{H} or the V_{L}, wherein the IL-2 molecule is a mutein, and wherein the antibody cytokine engrafted protein preferentially expands T effector cells over regulatory T cells. In some embodiments, the IL-2 regimen comprises administration of an antibody comprising a heavy chain selected from the group consisting of SEQ ID NO:29 and SEQ ID NO:38 and a light chain selected from the group consisting of SEQ ID NO:37 and SEQ ID NO:39, or a fragment, variant, or biosimilar thereof, every 1, 2, 4, 6, 7, 14 or 21 days at a dose of 0.10 mg/day to 50 mg/day

In some embodiments, the antibody cytokine engrafted protein described herein has a longer serum half-life than a wild-type IL-2 molecule such as, but not limited to, aldesleukin (Proleukin®) or a comparable molecule.

In some embodiments, the TIL infusion used with the foregoing embodiments of myeloablative lymphodepletion regimens may be any TIL composition described herein and may also include infusions of MILs and PBLs in place of the TIL infusion, as well as the addition of IL-2 regimens and administration of co-therapies (such as PD-1 and/or PD-L1 inhibitors and/or CTLA-4 inhibitors) as described herein.

### 5. Additional Methods of Treatment

In other embodiments, the invention provides a method for treating a subject with cancer comprising administering to the subject a therapeutically effective dosage of the therapeutic TIL population described in any one of the preceding paragraphs as applicable above.

In other embodiments, the invention provides a method for treating a subject with cancer comprising administering to the subject a therapeutically effective dosage of the TIL composition described in any of the preceding paragraph as applicable above.

In other embodiments, the invention provides the method for treating a subject with cancer described in any of the preceding paragraphs as applicable above modified such that prior to administering the therapeutically effective dosage of the therapeutic TIL population described in any of the preceding paragraphs as applicable above or the therapeutically effective dosage of the TIL composition described in any of the preceding paragraphs as applicable above, a non-myeloablative lymphodepletion regimen has been administered to the subject.

In other embodiments, the invention provides the method for treating a subject with cancer described in any of the preceding paragraph as applicable above modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In other embodiments, the invention provides the method for treating a subject with cancer described in any of the preceding paragraphs as applicable above modified to further comprise the step of treating the subject with a high-dose IL-2 regimen starting on the day after administration of the TIL cells to the subject.

In other embodiments, the invention provides the method for treating a subject with cancer described in any of the preceding paragraphs as applicable above modified such that the high-dose IL-2 regimen comprises 600,000 or 720,000 IU/kg administered as a 15-minute bolus intravenous infusion every eight hours until tolerance.

In other embodiments, the invention provides the method for treating a subject with cancer described in any of the preceding paragraphs as applicable above modified such that the cancer is a solid tumor.

In other embodiments, the invention provides the method for treating a subject with cancer described in any of the preceding paragraphs as applicable above modified such that the cancer is melanoma, ovarian cancer, endometrial cancer, thyroid cancer, colorectal cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In other embodiments, the invention provides the method for treating a subject with cancer described in any of the preceding paragraphs as applicable above modified such that the cancer is melanoma, HNSCC, cervical cancers, NSCLC, glioblastoma (including GBM), and gastrointestinal cancer.

In other embodiments, the invention provides the method for treating a subject with cancer described in any of the preceding paragraphs as applicable above modified such that the cancer is melanoma.

In other embodiments, the invention provides the method for treating a subject with cancer described in any of the preceding paragraphs as applicable above modified such that the cancer is HNSCC.

In other embodiments, the invention provides the method for treating a subject with cancer described in any of the preceding paragraphs as applicable above modified such that the cancer is a cervical cancer.

In other embodiments, the invention provides the method for treating a subject with cancer described in any of the preceding paragraphs as applicable above modified such that the cancer is NSCLC.

In other embodiments, the invention provides the method for treating a subject with cancer described in any of the preceding paragraphs as applicable above modified such that the cancer is glioblastoma (including GBM).

In other embodiments, the invention provides the method for treating a subject with cancer described in any of the preceding paragraphs as applicable above modified such that the cancer is gastrointestinal cancer.

In other embodiments, the invention provides the method for treating a subject with cancer described in any of the preceding paragraphs as applicable above modified such that the cancer is a hypermutated cancer.

In other embodiments, the invention provides the method for treating a subject with cancer described in any of the preceding paragraphs as applicable above modified such that the cancer is a pediatric hypermutated cancer.

In other embodiments, the invention provides the method for treating a subject with cancer described in any of the preceding paragraphs as applicable above modified such that the cancer is selected from the group consisting of anal cancer, bladder cancer, breast cancer (including triple-negative breast cancer), bone cancer, cancer caused by human papilloma virus (HPV), central nervous system associated cancer (including ependymoma, medulloblastoma, neuroblastoma, pineoblastoma, and primitive neuroectodermal tumor), cervical cancer (including squamous cell cervical cancer, adenosquamous cervical cancer, and cervical adenocarcinoma), colon cancer, colorectal cancer, endometrial cancer, esophageal cancer, esophagogastric junction cancer, gastric cancer, gastrointestinal cancer, gastrointestinal stromal tumor, glioblastoma, glioma, head and neck cancer (including head and neck squamous cell carcinoma (HNSCC), hypopharynx cancer, larynx cancer, nasopharynx cancer, oropharynx cancer, and pharynx cancer), kidney cancer, liver cancer, lung cancer (including non-small-cell lung cancer (NSCLC) and small-cell lung cancer), melanoma (including uveal melanoma, choroidal melanoma, ciliary body melanoma, or iris melanoma), mesothelioma (including malignant pleural mesothelioma), ovarian cancer, pancreatic cancer (including pancreatic ductal adenocarcinoma), penile cancer, rectal cancer, renal cancer, renal cell carcinoma, sarcoma (including Ewing sarcoma, osteosarcoma, rhabdomyosarcoma, and other bone and soft tissue sarcomas), thyroid cancer (including anaplastic thyroid cancer), uterine cancer, and vaginal cancer.

In other embodiments, the invention provides the therapeutic TIL population described in any one of the preceding paragraphs as applicable above for use in a method for treating a subject with cancer comprising administering to the subject a therapeutically effective dosage of the therapeutic TIL population.

In other embodiments, the invention provides the TIL composition described in any of the preceding paragraphs as applicable above for use in a method for treating a subject with cancer comprising administering to the subject a therapeutically effective dosage of the TIL composition.

In other embodiments, the invention provides the therapeutic TIL population described in any of the preceding paragraphs as applicable above or the TIL composition described in any of the preceding paragraphs as applicable above modified such that prior to administering to the subject the therapeutically effective dosage of the therapeutic TIL population described in any of the preceding paragraphs as applicable above or the TIL composition described in any of the preceding paragraphs as applicable above, a non-myeloablative lymphodepletion regimen has been administered to the subject.

In other embodiments, the invention provides the therapeutic TIL population or the TIL composition described in any of the preceding paragraphs as applicable above modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In other embodiments, the invention provides the therapeutic TIL population described in any of the preceding paragraphs as applicable above or the TIL composition described in any of the preceding paragraphs as applicable above modified to further comprise the step of treating patient with a high-dose IL-2 regimen starting on the day after administration of the TIL cells to the patient.

In other embodiments, the invention provides the therapeutic TIL population or the TIL composition described in any of the preceding paragraphs as applicable above modified such that the high-dose IL-2 regimen comprises 600,000 or 720,000 IU/kg administered as a 15-minute bolus intravenous infusion every eight hours until tolerance.

In other embodiments, the invention provides the therapeutic TIL population described in any of the preceding paragraphs as applicable above or the TIL composition described in any of the preceding paragraphs as applicable above modified such that the cancer is a solid tumor.

In other embodiments, the invention provides the therapeutic TIL population described in any of the preceding paragraphs as applicable above or the TIL composition described in any of the preceding paragraphs as applicable above modified such that the cancer is melanoma, ovarian cancer, endometrial cancer, thyroid cancer, colorectal cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In other embodiments, the invention provides the therapeutic TIL population described in any of the preceding paragraphs as applicable above or the TIL composition described in any of the preceding paragraphs as applicable above modified such that the cancer is melanoma, HNSCC, cervical cancers, NSCLC, glioblastoma (including GBM), and gastrointestinal cancer.

In other embodiments, the invention provides the therapeutic TIL population described in any of the preceding paragraphs as applicable above or the TIL composition described in any of the preceding paragraphs as applicable above modified such that the cancer is melanoma.

In other embodiments, the invention provides the therapeutic TIL population or the TIL composition described in any of the preceding paragraphs as applicable above modified such that the cancer is HNSCC.

In other embodiments, the invention provides the therapeutic TIL population or the TIL composition described in any of the preceding paragraphs as applicable above modified such that the cancer is a cervical cancer.

In other embodiments, the invention provides the therapeutic TIL population or the TIL composition described in any of the preceding paragraphs as applicable above modified such that the cancer is NSCLC.

In other embodiments, the invention provides the therapeutic TIL population or the TIL composition described in any of the preceding paragraphs as applicable above modified such that the cancer is glioblastoma (including GBM).

In other embodiments, the invention provides the therapeutic TIL population or the TIL composition described in any of the preceding paragraphs as applicable above modified such that the cancer is gastrointestinal cancer.

In other embodiments, the invention provides the therapeutic TIL population or the TIL composition described in any of the preceding paragraphs as applicable above modified such that the cancer is a hypermutated cancer.

In other embodiments, the invention provides the therapeutic TIL population or the TIL composition described in any of the preceding paragraphs as applicable above modified such that the cancer is a pediatric hypermutated cancer.

In other embodiments, the invention provides the therapeutic TIL population described in any of the preceding paragraphs as applicable above or the TIL composition described in any of the preceding paragraphs as applicable above modified such that the cancer is selected from the group consisting of anal cancer, bladder cancer, breast cancer (including triple-negative breast cancer), bone cancer, cancer caused by human papilloma virus (HPV), central nervous system associated cancer (including ependymoma, medulloblastoma, neuroblastoma, pineoblastoma, and primitive neuroectodermal tumor), cervical cancer (including squamous cell cervical cancer, adenosquamous cervical cancer, and cervical adenocarcinoma), colon cancer, colorectal cancer, endometrial cancer, esophageal cancer, esophagogastric junction cancer, gastric cancer, gastrointestinal cancer, gastrointestinal stromal tumor, glioblastoma, glioma, head and neck cancer (including head and neck squamous cell carcinoma (HNSCC), hypopharynx cancer, larynx cancer, nasopharynx cancer, oropharynx cancer, and pharynx cancer), kidney cancer, liver cancer, lung cancer (including non-small-cell lung cancer (NSCLC) and small-cell lung cancer), melanoma (including uveal melanoma, choroidal melanoma, ciliary body melanoma, or iris melanoma), mesothelioma (including malignant pleural mesothelioma), ovarian cancer, pancreatic cancer (including pancreatic ductal adenocarcinoma), penile cancer, rectal cancer, renal cancer, renal cell carcinoma, sarcoma (including Ewing sarcoma, osteosarcoma, rhabdomyosarcoma, and other bone and soft tissue sarcomas), thyroid cancer (including anaplastic thyroid cancer), uterine cancer, and vaginal cancer.

In other embodiments, the invention provides the use of the therapeutic TIL population described in any one of the preceding paragraphs as applicable above in a method of treating cancer in a subject comprising administering to the subject a therapeutically effective dosage of the therapeutic TIL population.

In other embodiments, the invention provides the use of the TIL composition described in any of the preceding paragraphs as applicable above in a method of treating cancer in a subject comprising administering to the subject a therapeutically effective dosage of the TIL composition.

In other embodiments, the invention provides the use of the therapeutic TIL population described in any of the preceding paragraphs as applicable above or the TIL composition described in any of the preceding paragraphs as applicable above in a method of treating cancer in a subject comprising administering to the subject a non-myeloablative lymphodepletion regimen and then administering to the subject a therapeutically effective dosage of the therapeutic TIL population described in any of the preceding paragraphs as applicable above or a therapeutically effective dosage of the TIL composition described in any of the preceding paragraphs as applicable above.

In other embodiments, the invention provides the use of the therapeutic TIL population or the TIL composition described in any of the preceding paragraphs as applicable above modified such that prior to administering to the subject the therapeutically effective dosage of the therapeutic TIL population or the therapeutically effective dosage of the TIL composition, a non-myeloablative lymphodepletion regimen has been administered to the subject.

In other embodiments, the invention provides the use of the therapeutic TIL population or the TIL composition described in any of the preceding paragraphs as applicable above modified such that the non-myeloablative lymphodepletion regimen comprises the steps of administration of cyclophosphamide at a dose of 60 mg/m²/day for two days followed by administration of fludarabine at a dose of 25 mg/m²/day for five days.

In other embodiments, the invention provides the use of the therapeutic TIL population described in any of the preceding paragraphs as applicable above or the use of the TIL composition described in any of the preceding paragraphs as applicable above modified to further comprise the step of treating patient with a high-dose IL-2 regimen starting on the day after administration of the TIL cells to the patient.

In other embodiments, the invention provides the use of the therapeutic TIL population or the TIL composition described in any of the preceding paragraphs as applicable above modified such that the high-dose IL-2 regimen comprises 600,000 or 720,000 IU/kg administered as a 15-minute bolus intravenous infusion every eight hours until tolerance.

In other embodiments, the invention provides the use of the therapeutic TIL population or the TIL composition described in any of the preceding paragraphs as applicable above modified such that the cancer is a solid tumor.

In other embodiments, the invention provides the use of the therapeutic TIL population or the TIL composition described in any of the preceding paragraphs as applicable above modified such that the cancer is melanoma, ovarian cancer, endometrial cancer, thyroid cancer, colorectal cancer, cervical cancer, non-small-cell lung cancer (NSCLC), lung cancer, bladder cancer, breast cancer, cancer caused by human papilloma virus, head and neck cancer (including head and neck squamous cell carcinoma (HNSCC)), glioblastoma (including GBM), gastrointestinal cancer, renal cancer, or renal cell carcinoma.

In other embodiments, the invention provides the use of the therapeutic TIL population or the TIL composition described in any of the preceding paragraphs as applicable above modified such that the cancer is melanoma, HNSCC, cervical cancers, NSCLC, glioblastoma (including GBM), and gastrointestinal cancer.

In other embodiments, the invention provides the use of the therapeutic TIL population or the TIL composition described in any of the preceding paragraphs as applicable above modified such that the cancer is melanoma.

In other embodiments, the invention provides the use of the therapeutic TIL population or the TIL composition described in any of the preceding paragraphs as applicable above modified such that the cancer is HNSCC.

In other embodiments, the invention provides the use of the therapeutic TIL population or the TIL composition described in any of the preceding paragraphs as applicable above modified such that the cancer is a cervical cancer.

In other embodiments, the invention provides the use of the therapeutic TIL population or the TIL composition described in any of the preceding paragraphs as applicable above modified such that the cancer is NSCLC.

In other embodiments, the invention provides the use of the therapeutic TIL population or the TIL composition described in any of the preceding paragraphs as applicable above modified such that the cancer is glioblastoma (including GBM).

In other embodiments, the invention provides the use of the therapeutic TIL population or the TIL composition described in any of the preceding paragraphs as applicable above modified such that the cancer is gastrointestinal cancer.

In other embodiments, the invention provides the use of the therapeutic TIL population or the TIL composition described in any of the preceding paragraphs as applicable above modified such that the cancer is a hypermutated cancer.

In other embodiments, the invention provides the use of the therapeutic TIL population or the TIL composition described in any of the preceding paragraphs as applicable above modified such that the cancer is a pediatric hypermutated cancer.

In other embodiments, the invention provides the use of the therapeutic TIL population or the TIL composition described in any of the preceding paragraphs as applicable above modified such that the cancer is selected from the group consisting of anal cancer, bladder cancer, breast cancer (including triple-negative breast cancer), bone cancer, cancer caused by human papilloma virus (HPV), central nervous system associated cancer (including ependymoma, medulloblastoma, neuroblastoma, pineoblastoma, and primitive neuroectodermal tumor), cervical cancer (including squamous cell cervical cancer, adenosquamous cervical cancer, and cervical adenocarcinoma), colon cancer, colorectal cancer, endometrial cancer, esophageal cancer, esophagogastric junction cancer, gastric cancer, gastrointestinal cancer, gastrointestinal stromal tumor, glioblastoma, glioma, head and neck cancer (including head and neck squamous cell carcinoma (HNSCC), hypopharynx cancer, larynx cancer, nasopharynx cancer, oropharynx cancer, and pharynx cancer), kidney cancer, liver cancer, lung cancer (including non-small-cell lung cancer (NSCLC) and small-cell lung cancer), melanoma (including uveal melanoma, choroidal melanoma, ciliary body melanoma, or iris melanoma), mesothelioma (including malignant pleural mesothelioma), ovarian cancer, pancreatic cancer (including pancreatic ductal adenocarcinoma), penile cancer, rectal cancer, renal cancer, renal cell carcinoma, sarcoma (including Ewing sarcoma, osteosarcoma, rhabdomyosarcoma, and other bone and soft tissue sarcomas), thyroid cancer (including anaplastic thyroid cancer), uterine cancer, and vaginal cancer.

### EXAMPLES

The embodiments encompassed herein are now described with reference to the following examples. These examples are provided for the purpose of illustration only and the disclosure encompassed herein should in no way be construed as being limited to these examples, but rather should be construed to encompass any and all variations which become evident as a result of the teachings provided herein.

### Example 1: Preparation of Media for Pre-Rep and Rep Processes

This example describes the procedure for the preparation of tissue culture media for use in protocols involving the culture of tumor infiltrating lymphocytes (TIL) derived from various solid tumors. This media can be used for preparation of any of the TILs described in the present application and other examples.

Preparation of CM1. Removed the following reagents from cold storage and warm them in a 37° C. water bath: (RPMI1640, Human AB serum, 200 mM L-glutamine). Prepared CM1 medium according to Table 42 below by adding each of the ingredients into the top section of a 0.2 μm filter unit appropriate to the volume to be filtered. Store at 4° C.

TABLE 42
Preparation of CM1

| | Final | | Final Volume | Final Volume |
|---|---|---|---|---|
| Ingredient | concentration | | 500 mL | IL |
| RPMI1640 | NA | | 450 mL | 900 mL |
| Human AB serum, | 50 | mL | 100 mL | |
| heat-inactivated 10% | | | | |
| 200 mM L-glutamine | 2 | mM | 5 mL | 10 mL |
| 55 mM BME | 55 | μM | 0.5 mL | 1 mL |
| 50 mg/mL | 50 | μg/mL | 0.5 mL | 1 mL |
| gentamicin sulfate | | | | |

On the day of use, prewarmed required amount of CM1 in 37° C. water bath and add 6000 IU/mL IL-2.

Additional supplementation may be performed as needed according to Table 43.

TABLE 43
Additional supplementation of CM1, as needed.

| | Stock | | | Final | |
|---|---|---|---|---|---|
| Supplement | concentration | | Dilution | concentration | |
| GlutaMAX ™ | 200 | mM | 1:100 | 2 | mM |
| Penicillin/ | 10,000 U/mL | | 1:100 | 100 U/mL | |
| streptomycin | penicillin | | | penicillin | |
| | 10,000 μg/mL | | | 100 μg/mL | |
| | streptomycin | | | streptomycin | |
| Amphotericin B | 250 | μg/mL | 1:100 | 2.5 | μg/mL |

### Preparation of CM2

Removed prepared CM1 from refrigerator or prepare fresh CM1. Removed AIM-V® from refrigerator and prepared the amount of CM2 needed by mixing prepared CM1 with an equal volume of AIM-V® in a sterile media bottle. Added 3000 IU/mL IL-2 to CM2 medium on the day of usage. Made sufficient amount of CM2 with 3000 IU/mL IL-2 on the day of usage. Labeled the CM2 media bottle with its name, the initials of the preparer, the date it was filtered/prepared, the two-week expiration date and store at 4° C. until needed for tissue culture.

### Preparation of CM3

Prepared CM3 on the day it was required for use. CM3 was the same as AIM-V® medium, supplemented with 3000 IU/mL IL-2 on the day of use. Prepared an amount of CM3 sufficient to experimental needs by adding IL-2 stock solution directly to the bottle or bag of AIM-V. Mixed well by gentle shaking. Label bottle with “3000 IU/mL IL-2” immediately after adding to the AIM-V. If there was excess CM3, stored it in bottles at 4° C. labeled with the media name, the initials of the preparer, the date the media was prepared, and its expiration date (7 days after preparation). Discarded media supplemented with IL-2 after 7 days storage at 4° C.

### Preparation of CM4

CM4 was the same as CM3, with the additional supplement of 2 mM GlutaMAX™ (final concentration). For every 1 L of CM3, add 10 mL of 200 mM GlutaMAX™. Prepare an amount of CM4 sufficient to experimental needs by adding IL-2 stock solution and GlutaMAX™ stock solution directly to the bottle or bag of AIM-V. Mixed well by gentle shaking. Labeled bottle with “3000 IL/mL IL-2 and GlutaMAX” immediately after adding to the AIM-V. If there was excess CM4, stored it in bottles at 4° C. labeled with the media name, “GlutaMAX”, and its expiration date (7 days after preparation). Discarded media supplemented with IL-2 after more than 7-days storage at 4° C.

### Example 2: Use of IL-2, IL-15, AND IL-21 Cytokine Cocktail

This example describes the use of IL-2, IL-15, and IL-21 cytokines, which serve as additional T cell growth factors, in combination with the TIL process of any of the examples herein.

Using the processes described herein, TILs can be grown from tumors in presence of IL-2 in one arm of the experiment and, in place of IL-2, a combination of IL-2, IL-15, and IL-21 in another arm at the initiation of culture. At the completion of the pre-REP, cultures were assessed for expansion, phenotype, function (CD107a+ and IFN-γ) and TCR Vβ repertoire. IL-15 and IL-21 are described elsewhere herein and in Santegoets, et al., J. Transl. Med., 2013,11, 37.

The results can show that enhanced TIL expansion (>20%), in both CD4⁺ and CD8⁺ cells in the IL-2, IL-15, and IL-21 treated conditions can observed relative to the IL-2 only conditions. There was a skewing towards a predominantly CD8⁺ population with a skewed TCR Vβ repertoire in the TILs obtained from the IL-2, IL-15, and IL-21 treated cultures relative to the IL-2 only cultures. IFN-γ and CD107a were elevated in the IL-2, IL-15, and IL-21 treated TILs, in comparison to TILs treated only IL-2.

### Example 3: Qualifying Individual Lots of Gamma-Irradiated Peripheral Mononuclear Cells

This Example describes an abbreviated procedure for qualifying individual lots of gamma-irradiated peripheral mononuclear cells (PBMCs, also known as mononuclear cells or MNCs) for use as allogeneic feeder cells in the exemplary methods described herein.

Each irradiated MNC feeder lot was prepared from an individual donor. Each lot or donor was screened individually for its ability to expand TIL in the REP in the presence of purified anti-CD3 (clone OKT3) antibody and interleukin-2 (IL-2). In addition, each lot of feeder cells was tested without the addition of TIL to verify that the received dose of gamma radiation was sufficient to render them replication incompetent.

Gamma-irradiated, growth-arrested MNC feeder cells are required for REP of TILs. Membrane receptors on the feeder MNCs bind to anti-CD3 (clone OKT3) antibody and crosslink to TILs in the REP flask, stimulating the TIL to expand. Feeder lots were prepared from the leukapheresis of whole blood taken from individual donors. The leukapheresis product was subjected to centrifugation over Ficoll-Hypaque, washed, irradiated, and cryopreserved under GMP conditions.

It is important that patients who received TIL therapy not be infused with viable feeder cells as this can result in graft-versus-host disease (GVHD). Feeder cells are therefore growth-arrested by dosing the cells with gamma-irradiation, resulting in double strand DNA breaks and the loss of cell viability of the MNC cells upon re-culture.

Feeder lots were evaluated on two criteria: (1) their ability to expand TILs in co-culture >100-fold and (2) their replication incompetency. Feeder lots were tested in mini-REP format utilizing two primary pre-REP TIL lines grown in upright T25 tissue culture flasks. Feeder lots were tested against two distinct TIL lines, as each TIL line is unique in its ability to proliferate in response to activation in a REP. As a control, a lot of irradiated MNC feeder cells which has historically been shown to meet the criteria above was run alongside the test lots.

To ensure that all lots tested in a single experiment receive equivalent testing, sufficient stocks of the same pre-REP TIL lines were available to test all conditions and all feeder lots.

For each lot of feeder cells tested, there was a total of six T25 flasks: Pre-REP TIL line #1 (2 flasks); Pre-REP TIL line #2 (2 flasks); and feeder control (2 flasks). Flasks containing TIL lines #1 and #2 evaluated the ability of the feeder lot to expand TIL. The feeder control flasks evaluated the replication incompetence of the feeder lot.

### A. Experimental Protocol

Day −2/3, Thaw of TIL lines. Prepare CM2 medium and warm CM2 in 37° C. water bath. Prepare 40 mL of CM2 supplemented with 3000 IU/mL IL-2. Keep warm until use. Place 20 mL of pre-warmed CM2 without IL-2 into each of two 50 mL conical tubes labeled with names of the TIL lines used. Removed the two designated pre-REP TIL lines from LN2 storage and transferred the vials to the tissue culture room. Thawed vials by placing them inside a sealed zipper storage bag in a 37° C. water bath until a small amount of ice remains.

Using a sterile transfer pipet, the contents of each vial were immediately transferred into the 20 mL of CM2 in the prepared, labeled 50 mL conical tube. QS to 40 mL using CM2 without IL-2 to wash cells and centrifuged at 400×CF for 5 minutes. Aspirated the supernatant and resuspend in 5 mL warm CM2 supplemented with 3000 IU/mL IL-2.

A small aliquot (20 μL) was removed in duplicate for cell counting using an automated cell counter. The counts were recorded. While counting, the 50 mL conical tube with TIL cells was placed into a humidified 37° C., 5% CO₂ incubator, with the cap loosened to allow for gas exchange. The cell concentration was determined, and the TILs were diluted to 1×10⁶ cells/mL in CM2 supplemented with IL-2 at 3000 IU/mL.

Cultured in 2 mL/well of a 24-well tissue culture plate in as many wells as needed in a humidified 37° C. incubator until Day 0 of the mini-REP. The different TIL lines were cultured in separate 24-well tissue culture plates to avoid confusion and potential cross-contamination.

Day 0, initiate Mini-REP. Prepared enough CM2 medium for the number of feeder lots to be tested. (e.g., for testing 4 feeder lots at one time, prepared 800 mL of CM2 medium). Aliquoted a portion of the CM2 prepared above and supplemented it with 3000 IU/mL IL-2 for the culturing of the cells. (e.g., for testing 4 feeder lots at one time, prepare 500 mL of CM2 medium with 3000 IU/mL IL-2).

Working with each TIL line separately to prevent cross-contamination, the 24-well plate with TIL culture was removed from the incubator and transferred to the BSC.

Using a sterile transfer pipet or 100-1000 μL pipettor and tip, about 1 mL of medium was removed from each well of TILs to be used and placed in an unused well of the 24-well tissue culture plate.

Using a fresh sterile transfer pipet or 100-1000 μL pipettor and tip, the remaining medium was mixed with TILs in wells to resuspend the cells and then transferred the cell suspension to a 50 mL conical tube labeled with the TIL lot name and recorded the volume.

Washed the wells with the reserved media and transferred that volume to the same 50 mL conical tube. Spun the cells at 400×CF to collect the cell pellet. Aspirated off the media supernatant and resuspend the cell pellet in 2-5 mL of CM2 medium containing 3000 IU/mL IL-2, volume to be used based on the number of wells harvested and the size of the pellet—volume should be sufficient to ensure a concentration of >1.3×10⁶ cells/mL.

Using a serological pipet, the cell suspension was mixed thoroughly and the volume was recorded. Removed 200 μL for a cell count using an automated cell counter. While counting, placed the 50 mL conical tube with TIL cells into a humidified, 5% CO₂, 37° C. incubator, with the cap loosened to allow gas exchange. Recorded the counts.

Removed the 50 mL conical tube containing the TIL cells from the incubator and resuspend them cells at a concentration of 1.3×10⁶ cells/mL in warm CM2 supplemented with 3000 IU/mL IL-2. Returned the 50 mL conical tube to the incubator with a loosened cap.

The steps above were repeated for the second TIL line.

Just prior to plating the TIL into the T25 flasks for the experiment, TIL were diluted 1:10 for a final concentration of 1.3×10⁵ cells/mL as per below.

Prepare MACS GMP CD3 pure (OKT3) working solution. Took out stock solution of OKT3 (1 mg/mL) from 4° C. refrigerator and placed in BSC. A final concentration of 30 ng/mL OKT3 was used in the media of the mini-REP.

600 ng of OKT3 were needed for 20 mL in each T25 flask of the experiment; this was the equivalent of 60 μL of a 10 μg/mL solution for each 20 mL, or 360 μL for all 6 flasks tested for each feeder lot.

For each feeder lot tested, made 400 μL of a 1:100 dilution of 1 mg/mL OKT3 for a working concentration of 10 μg/mL (e.g., for testing 4 feeder lots at one time, make 1600 μL of a 1:100 dilution of 1 mg/mL OKT3: 16 μL of 1 mg/mL OKT3+1.584 mL of CM2 medium with 3000 IU/mL IL-2.)

Prepare T25 flasks. Labeled each flask and filled flask with the CM2 medium prior to preparing the feeder cells. Placed flasks into 37° C. humidified 5% CO₂ incubator to keep media warm while waiting to add the remaining components. Once feeder cells were prepared, the components will be added to the CM2 in each flask.

Further information is provided in Table 44.

TABLE 44
Solution information.

| | | Volume in |
|---|---|---|
| | Volume in co- | control (feeder |
| Component | culture flasks | only) flasks |
| CM2 + 3000 IU/mL IL-2 | 18 mL | 19 mL |
| MNC: 1.3 × 10⁷/mL in CM2 + 3000 | 1 mL | 1 mL |
| IU IL-2 | | |
| (final concentration 1.3 × 10⁷/flask) | | |
| OKT3: 10 μL/mL in CM2 = 3000 IU | 60 μL | 60 μL |
| IL-2 | | |
| TIL: 1.3 × 10⁵/mL in CM2 with 3000 | 1 mL | 0 |
| IU of IL-2 | | |
| (final concentration 1.3 × 10⁵/flask) | | |

Prepare Feeder Cells. A minimum of 78×10⁶ feeder cells were needed per lot tested for this protocol. Each 1 mL vial frozen by SDBB had 100×10⁶ viable cells upon freezing. Assuming a 50% recovery upon thaw from liquid N₂ storage, it was recommended to thaw at least two 1 mL vials of feeder cells per lot giving an estimated 100×10⁶ viable cells for each REP. Alternately, if supplied in 1.8 mL vials, only one vial provided enough feeder cells.

Before thawing feeder cells, approximately 50 mL of CM2 without IL-2 was pre-warmed for each feeder lot to be tested. The designated feeder lot vials were removed from LN2 storage, placed in zipper storage bag, and placed on ice. Vials were thawed inside closed zipper storage bag by immersing in a 37° C. water bath. Vials were removed from zipper bag, sprayed or wiped with 70% EtOH, and transferred to a BSC.

Using a transfer pipet, the contents of feeder vials were immediately transferred into 30 mL of warm CM2 in a 50 mL conical tube. The vial was washed with a small volume of CM2 to remove any residual cells in the vial and centrifuged at 400×CF for 5 minutes. Aspirated the supernatant and resuspended in 4 mL warm CM2 plus 3000 IU/mL IL-2. Removed 200 μL for cell counting using the automated cell counter. Recorded the counts.

Resuspended cells at 1.3×10⁷ cells/mL in warm CM2 plus 3000 IU/mL IL-2. Diluted TIL cells from 1.3×10⁶ cells/mL to 1.3×10⁵ cells/mL.

Setup Co-Culture. Diluted TIL cells from 1.3×10⁶ cells/mL to 1.3×10⁵ cells/mL. Added 4.5 mL of CM2 medium to a 15 mL conical tube. Removed TIL cells from incubator and resuspended well using a 10 mL serological pipet. Removed 0.5 mL of cells from the 1.3×10⁶ cells/mL TIL suspension and added to the 4.5 mL of medium in the 15 mL conical tube. Returned TIL stock vial to incubator. Mixed well. Repeated for the second TIL line.

Transferred flasks with pre-warmed media for a single feeder lot from the incubator to the BSC. Mixed feeder cells by pipetting up and down several times with a 1 mL pipet tip and transferred 1 mL (1.3×10⁷ cells) to each flask for that feeder lot. Added 60 μL of OKT3 working stock (10 μg/mL) to each flask. Returned the two control flasks to the incubator.

Transferred 1 mL (1.3×10⁵) of each TIL lot to the correspondingly labeled T25 flask. Returned flasks to the incubator and incubate upright. Did not disturb until Day 5. This procedure was repeated for all feeder lots tested.

Day 5, Media change. Prepared CM2 with 3000 IU/mL IL-2. 10 mL is needed for each flask. With a 10 mL pipette, transferred 10 mL warm CM2 with 3000 IU/mL IL-2 to each flask. Returned flasks to the incubator and incubated upright until day 7. Repeated for all feeder lots tested.

Day 7, Harvest. Removed flasks from the incubator and transfer to the BSC, care as taken not to disturb the cell layer on the bottom of the flask. Without disturbing the cells growing on the bottom of the flasks, 10 mL of medium was removed from each test flask and 15 mL of medium from each of the control flasks.

Using a 10 mL serological pipet, the cells were resuspended in the remaining medium and mix well to break up any clumps of cells. After thoroughly mixing cell suspension by pipetting, removed 200 μL for cell counting. Counted the TIL using the appropriate standard operating procedure in conjunction with the automatic cell counter equipment. Recorded counts in day 7. This procedure was repeated for all feeder lots tested.

Feeder control flasks were evaluated for replication incompetence and flasks containing TIL were evaluated for fold expansion from day 0.

Day 7, Continuation of Feeder Control Flasks to Day 14. After completing the day 7 counts of the feeder control flasks, 15 mL of fresh CM2 medium containing 3000 IU/mL IL-2 was added to each of the control flasks. The control flasks were returned to the incubator and incubated in an upright position until day 14.

Day 14, Extended Non-proliferation of Feeder Control Flasks. Removed flasks from the incubator and transfer to the BSC, care was taken not to disturb the cell layer on the bottom of the flask. Without disturbing the cells growing on the bottom of the flasks, approximately 17 mL of medium was removed from each control flasks. Using a 5 mL serological pipet, the cells were resuspended in the remaining medium and mixed well to break up any clumps of cells. The volumes were recorded for each flask.

After thoroughly mixing the cell suspension by pipetting, 200 μL was removed for cell counting. The TIL were counted using the appropriate standard operating procedure in conjunction with the automatic cell counter equipment and the counts were recorded. This procedure was repeated for all feeder lots tested.

### B. Results and Acceptance Criteria Protocol

Results. The dose of gamma irradiation was sufficient to render the feeder cells replication incompetent. All lots were expected to meet the evaluation criteria and also demonstrated a reduction in the total viable number of feeder cells remaining on day 7 of the REP culture compared to day 0. All feeder lots were expected to meet the evaluation criteria of 100-fold expansion of TIL growth by day 7 of the REP culture. Day 14 counts of Feeder Control flasks were expected to continue the non-proliferative trend seen on day 7.

Acceptance Criteria. The following acceptance criteria were met for each replicate TIL line tested for each lot of feeder cells. Acceptance criteria were two-fold, as shown in Table 45 below.

TABLE 45
Embodiments of acceptance criteria.

| Test | Acceptance criteria |
|---|---|
| Irradiation of MNC and | No growth observed at 7 and |
| Replication Incompetence | 14 days |
| TIL expansion | At least a 100-fold expansion of |
| | each TIL (minimum of 1.3 × 10⁷ |
| | viable cells) |

The dose of radiation was evaluated for its sufficiency to render the MNC feeder cells replication incompetent when cultured in the presence of 30 ng/mL OKT3 antibody and 3000 IU/mL IL-2. Replication incompetence was evaluated by total viable cell count (TVC) as determined by automated cell counting on day 7 and day 14 of the REP.

The acceptance criteria was “No Growth,” meaning the total viable cell number has not increased on day 7 and day 14 from the initial viable cell number put into culture on Day 0 of the REP.

The ability of the feeder cells to support TIL expansion was evaluated. TIL growth was measured in terms of fold expansion of viable cells from the onset of culture on day 0 of the REP to day 7 of the REP. On day 7, TIL cultures achieved a minimum of 100-fold expansion, (i.e., greater than 100 times the number of total viable TIL cells put into culture on REP day 0), as evaluated by automated cell counting.

Contingency Testing of MNC Feeder Lots that do not meet acceptance criteria. In the event that an MNC feeder lot did not meet the either of the acceptance criteria outlined above, the following steps will be taken to retest the lot to rule out simple experimenter error as its cause.

If there are two or more remaining satellite testing vials of the lot, then the lot was retested. If there were one or no remaining satellite testing vials of the lot, then the lot was failed according to the acceptance criteria listed above.

In order to be qualified, the lot in question and the control lot had to achieve the acceptance criteria above. Upon meeting these criteria, the lot is released for use.

### Example 4: Preparation of IL-2 Stock Solution

This Example describes the process of dissolving purified, lyophilized recombinant human interleukin-2 into stock samples suitable for use in further tissue culture protocols, including all of those described in the present application and Examples, including those that involve using rhIL-2.

Procedure. Prepared 0.2% Acetic Acid solution (HAc). Transferred 29 mL sterile water to a 50 mL conical tube. Added 1 mL 1N acetic acid to the 50 mL conical tube. Mixed well by inverting tube 2-3 times. Sterilized the HAc solution by filtration using a Steriflip filter.

Prepare 1% HSA in PBS. Added 4 mL of 25% HSA stock solution to 96 mL PBS in a 150 mL sterile filter unit. Filtered solution. Stored at 4° C. For each vial of rhIL-2 prepared, fill out forms.

Prepared rhIL-2 stock solution (6×10⁶ IU/mL final concentration). Each lot of rhIL-2 was different and required information found in the manufacturer's Certificate of Analysis (COA), such as: 1) Mass of rhIL-2 per vial (mg), 2) Specific activity of rhIL-2 (IU/mg) and 3) Recommended 0.2% HAc reconstitution volume (mL).

Calculated the volume of 1% HSA required for rhIL-2 lot by using the equation below:

$$( \frac{Vial Mass ( mg ) \times Biological Activity ( \frac{IU}{mg} )}{6 \times {10}^{6} \frac{IU}{mL}} ) - HAc vol ( mL ) = 1 \% HSA vol ( mL )$$

For example, according to the COA of rhIL-2 lot 10200121 (Cellgenix), the specific activity for the 1 mg vial is 25×10⁶ IU/mg. It recommends reconstituting the rhIL-2 in 2 mL 0.2% HAc.

$$( \frac{1 mg \times 25 \times {10}^{6} \frac{IU}{mg}}{6 \times {10}^{6} \frac{IU}{mL}} ) - 2 mL = 2.167 mL HSA$$

Wiped rubber stopper of IL-2 vial with alcohol wipe. Using a 16G needle attached to a 3 mL syringe, injected recommended volume of 0.2% HAc into vial. Took care to not dislodge the stopper as the needle is withdrawn. Inverted vial 3 times and swirled until all powder is dissolved. Carefully removed the stopper and set aside on an alcohol wipe. Added the calculated volume of 1% HSA to the vial.

Storage of rhIL-2 solution. For short-term storage (<72 hrs), stored vial at 4° C. For long-term storage (>72 hrs), aliquoted vial into smaller volumes and stored in cryovials at −20° C. until ready to use. Avoided freeze/thaw cycles. Expired 6 months after date of preparation. Rh-IL-2 labels included vendor and catalog number, lot number, expiration date, operator initials, concentration and volume of aliquot.

### Example 5: Cryopreservation Process

This example describes a cryopreservation process method for TILs prepared with the procedures described herein using the CryoMed Controlled Rate Freezer, Model 7454 (Thermo Scientific).

The equipment used was as follows: aluminum cassette holder rack (compatible with CS750 freezer bags), cryostorage cassettes for 750 mL bags, low pressure (22 psi) liquid nitrogen tank, refrigerator, thermocouple sensor (ribbon type for bags), and CryoStore CS750 freezing bags (OriGen Scientific).

The freezing process provides for a 0.5° C. rate from nucleation to −20° C. and 1° C. per minute cooling rate to −80° C. end temperature. The program parameters are as follows: Step 1—wait at 4° C.; Step 2: 1.0° C./min (sample temperature) to −4° C.; Step 3: 20.0° C./min (chamber temperature) to −45° C.; Step 4: 10.0° C./min (chamber temperature) to −10.0° C.; Step 5: 0.5° C./min (chamber temperature) to −20° C.; and Step 6: 1.0° C./min (sample temperature) to −80° C.

### Example 6: Tumor Expansion Processes with Defined Medium

The processes disclosed above or below may be performed substituting the CM1 and CM2 media with a defined medium according (e.g., CTS™ OpTmizer™ T-Cell Expansion SFM, ThermoFisher, including for example DM1 and DM2).

### Example 7: Exemplary Gen 2 Production of a Cryopreserved TIL Cell Therapy

This examples describes the cGMP manufacture of Iovance Biotherapeutics, Inc. TIL Cell Therapy Process in G-REX Flasks according to current Good Tissue Practices and current Good Manufacturing Practices. This example describes an exemplary cGMP manufacture of TIL Cell Therapy Process in G-REX Flasks according to current Good Tissue Practices and current Good Manufacturing Practices.

TABLE 46
Process Expansion Exemplary Plan.

| Estimated Day | | Target | Anticipated | Estimated Total |
|---|---|---|---|---|
| (post-seed) | Activity | Criteria | Vessels | Volume (mL) |
| 0 | Tumor Dissection | ≤50 desirable tumor fragments | G-REX-100MCS 1 flask | ≤1000 |
| | | per G-REX-100MCS | | |
| 11 | REP Seed | 5 − 200 × 10⁶viable | G-REX-500MCS 1 flasks | ≤5000 |
| | | cells per G-REX-500MCS | | |
| 16 | REP Split | 1 × 10⁹viable cells | G-REX-500MCS ≤5 flasks | ≤25000 |
| | | per G-REX-500MCS | | |
| 22 | Harvest | Total available cells | 3-4 CS-750 bags | ≤530 |

TABLE 47
Flask Volumes.

| | Working |
|---|---|
| | Volume/Flask |
| Flask Type | (mL) |
| G-REX-100MCS | 1000 |
| G-REX-500MCS | 5000 |

Day 0 CM1 Media Preparation. In the BSC added reagents to RPMI 1640 Media bottle. Added the following reagents t Added per bottle: Heat Inactivated Human AB Serum (100.0 mL); GlutaMax™ (10.0 mL); Gentamicin sulfate, 50 mg/mL (1.0 mL); 2-mercaptoethanol (1.0 mL)

Removed unnecessary materials from BSC. Passed out media reagents from BSC, left Gentamicin Sulfate and HBSS in BSC for Formulated Wash Media preparation.

Thawed IL-2 aliquot. Thawed one 1.1 mL IL-2 aliquot (6×10⁶ IU/mL) (BR71424) until all ice had melted. Recorded IL-2: Lot # and Expiry

Transferred IL-2 stock solution to media. In the BSC, transferred 1.0 mL of IL-2 stock solution to the CM1 Day 0 Media Bottle prepared. Added CM1 Day 0 Media 1 bottle and IL-2 (6×10⁶ IU/mL) 1.0 mL.

Passed G-REX100MCS into BSC. Aseptically passed G-REX100MCS (W3013130) into the BSC.

Pumped all Complete CM1 Day 0 Media into G-REX100MCS flask. Tissue Fragments Conical or GRex100MCS.

Day 0 Tumor Wash Media Preparation. In the BSC, added 5.0 mL Gentamicin (W3009832 or W3012735) to 1×500 mL HBSS Media (W3013128) bottle. Added per bottle: HBSS (500.0 mL); Gentamicin sulfate, 50 mg/mL (5.0 mL). Filtered HBSS containing gentamicin prepared through a 1 L 0.22-micron filter unit (W1218810).

Day 0 Tumor Processing. Obtained tumor specimen and transferred into suite at 2-8° C. immediately for processing. Aliquoted tumor wash media. Tumor wash 1 is performed using 8″ forceps (W3009771). The tumor is removed from the specimen bottle and transferred to the “Wash 1” dish prepared. This is followed by tumor wash 2 and tumor wash 3. Measured and assessed tumor. Assessed whether >30% of entire tumor area observed to be necrotic and/or fatty tissue. Clean up dissection if applicable. If tumor was large and >30% of tissue exterior was observed to be necrotic/fatty, performed “clean up dissection” by removing necrotic/fatty tissue while preserving tumor inner structure using a combination of scalpel and/or forceps. Dissect tumor. Using a combination of scalpel and/or forceps, cut the tumor specimen into even, appropriately sized fragments (up to 6 intermediate fragments). Transferred intermediate tumor fragments. Dissected tumor fragments into pieces approximately 3×3×3 mm in size. Stored Intermediate Fragments to prevent drying. Repeated intermediate fragment dissection. Determined number of pieces collected. If desirable tissue remains, selected additional favorable tumor pieces from the “favorable intermediate fragments” 6-well plate to fill the drops for a maximum of 50 pieces.

Prepared conical tube. Transferred tumor pieces to 50 mL conical tube. Prepared BSC for G-REX100MCS. Removed G-REX100MCS from incubator. Aseptically passed G-REX100MCS flask into the BSC. Added tumor fragments to G-REX100MCS flask. Evenly distributed pieces.

Incubated G-REX100MCS at the following parameters: Incubated G-REX flask: Temperature LED Display: 37.0±2.0° C.; CO₂ Percentage: 5.0±1.5% CO₂. Calculations: Time of incubation; lower limit=time of incubation+252 hours; upper limit=time of incubation+276 hours.

After process was complete, discarded any remaining warmed media and thawed aliquots of IL-2.

Day 11—Media Preparation. Monitored incubator. Incubator parameters: Temperature LED Display: 37.0±2.0° C.; CO2 Percentage: 5.0±1.5% CO2.

Warmed 3×1000 mL RPMI 1640 Media (W3013112) bottles and 3×1000 mL AIM-V (W3009501) bottles in an incubator for ≥30 minutes. Removed RPMI 1640 Media from incubator. Prepared RPMI 1640 Media. Filter Media. Thawed 3×1.1 mL aliquots of IL-2 (6×10⁶ IU/mL) (BR71424). Removed AIM-V Media from the incubator. Add IL-2 to AIM-V. Aseptically transferred a 10 L Labtainer Bag and a repeater pump transfer set into the BSC.

Prepared 10 L Labtainer media bag. Prepared Baxa pump. Prepared 10 L Labtainer media bag. Pumped media into 10 L Labtainer. Removed pumpmatic from Labtainer bag.

Mixed media. Gently massaged the bag to mix. Sample media per sample plan. Removed 20.0 mL of media and place in a 50 mL conical tube. Prepared cell count dilution tubes. In the BSC, added 4.5 mL of AIM-V Media that had been labelled with “For Cell Count Dilutions” and lot number to four 15 mL conical tubes. Transferred reagents from the BSC to 2-8° C. Prepared 1 L Transfer Pack. Outside of the BSC weld (per Process Note 5.11) a 1 L Transfer Pack to the transfer set attached to the “Complete CM2 Day 11 Media” bag prepared. Prepared feeder cell transfer pack. Incubated Complete CM2 Day 11 Media.

Day 11—TIL Harvest. Preprocessing table. Incubator parameters: Temperature LED display: 37.0±2.0° C.; CO₂ Percentage: 5.0±1.5% CO₂. Removed G-REX100MCS from incubator. Prepared 300 mL Transfer Pack. Welded transfer packs to G-REX100MCS.

Prepare flask for TIL Harvest and initiation of TIL Harvest. TIL Harvested. Using the GatheRex, transferred the cell suspension through the blood filter into the 300 mL transfer pack. Inspect membrane for adherent cells.

Rinsed flask membrane. Closed clamps on G-REX100MCS. Ensured all clamps are closed. Heat sealed the TIL and the “Supernatant” transfer pack. Calculated volume of TIL suspension. Prepared Supernatant Transfer Pack for Sampling.

Pulled Bac-T Sample. In the BSC, draw up approximately 20.0 mL of supernatant from the 1 L “Supernatant” transfer pack and dispense into a sterile 50 mL conical tube.

Inoculated BacT per Sample Plan. Removed a 1.0 mL sample from the 50 mL conical labeled BacT prepared using an appropriately sized syringe and inoculated the anaerobic bottle.

Incubated TIL. Placed TIL transfer pack in incubator until needed. Performed cell counts and calculations. Determined the Average of Viable Cell Concentration and Viability of the cell counts performed. Viability÷2. Viable Cell Concentration÷2. Determined Upper and Lower Limit for counts. Lower Limit: Average of Viable Cell Concentration ×0.9. Upper Limit: Average of Viable Cell Concentration ×1.1. Confirmed both counts within acceptable limits. Determined an average Viable Cell Concentration from all four counts performed.

Adjusted Volume of TIL Suspension: Calculate the adjusted volume of TIL suspension after removal of cell count samples. Total TIL Cell Volume (A). Volume of Cell Count Sample Removed (4.0 mL) (B) Adjusted Total TIL Cell Volume C=A−B.

Calculated Total Viable TIL Cells. Average Viable Cell Concentration*: Total Volume; Total Viable Cells: C=A×B.

Calculation for flow cytometry: if the Total Viable TIL Cell count from was ≥4.0×10⁷, calculated the volume to obtain 1.0×10⁷ cells for the flow cytometry sample.

Total viable cells required for flow cytometry: 1.0×10⁷ cells. Volume of cells required for flow cytometry: Viable cell concentration divided by 1.0×10⁷ cells A.

Calculated the volume of TIL suspension equal to 2.0×10⁸ viable cells. As needed, calculated the excess volume of TIL cells to remove and removed excess TIL and placed TIL in incubator as needed. Calculated total excess TIL removed, as needed.

Calculated amount of CS-10 media to add to excess TIL cells with the target cell concentration for freezing is 1.0×10⁸ cells/mL. Centrifuged excess TILs, as needed. Observed conical tube and added CS-10.

Filled Vials. Aliquoted 1.0 mL cell suspension, into appropriately sized cryovials. Aliquoted residual volume into appropriately sized cryovial. If volume is ≤0.5 mL, add CS10 to vial until volume is 0.5 mL.

Calculated the volume of cells required to obtain 1×10⁷ cells for cryopreservation. Removed sample for cryopreservation. Placed TIL in incubator.

Cryopreservation of sample. Observed conical tube and added CS-10 slowly and record volume of 0.5 mL of CS10 added.

Day 11—Feeder Cells. Obtained feeder cells. Obtained 3 bags of feeder cells with at least two different lot numbers from LN2 freezer. Kept cells on dry ice until ready to thaw. Prepared water bath or cryotherm. Thawed feeder cells at 37.0±2.0° C. in the water bath or cytotherm for ˜3-5 minutes or until ice has just disappeared. Removed media from incubator. Pooled thawed feeder cells. Added feeder cells to transfer pack. Dispensed the feeder cells from the syringe into the transfer pack. Mixed pooled feeder cells and labeled transfer pack.

Calculated total volume of feeder cell suspension in transfer pack. Removed cell count samples. Using a separate 3 mL syringe for each sample, pulled 4×1.0 mL cell count samples from Feeder Cell Suspension Transfer Pack using the needless injection port. Aliquoted each sample into the cryovials labeled. Performed cell counts and determine multiplication factors, elected protocols and entered multiplication factors. Determined the average of viable cell concentration and viability of the cell counts performed. Determined upper and lower limit for counts and confirm within limits.

Adjusted volume of feeder cell suspension. Calculated the adjusted volume of feeder cell suspension after removal of cell count samples. Calculated total viable feeder cells. Obtained additional feeder cells as needed. Thawed additional feeder cells as needed. Placed the 4th feeder cell bag into a zip top bag and thaw in a 37.0±2.0° C. water bath or cytotherm for ˜3-5 minutes and pooled additional feeder cells. Measured volume. Measured the volume of the feeder cells in the syringe and recorded below (B). Calculated the new total volume of feeder cells. Added feeder cells to transfer pack.

Prepared dilutions as needed, adding 4.5 mL of AIM-V Media to four 15 mL conical tubes. Prepared cell counts. Using a separate 3 mL syringe for each sample, removed 4×1.0 mL cell count samples from Feeder Cell Suspension transfer pack, using the needless injection port. Performed cell counts and calculations. Determined an average viable cell concentration from all four counts performed. Adjusted volume of feeder cell suspension and calculated the adjusted volume of feeder cell suspension after removal of cell count samples. Total Feeder Cell Volume minues 4.0 mL removed. Calculated the volume of Feeder Cell Suspension that was required to obtain 5×10⁹ viable feeder cells. Calculated excess feeder cell volume. Calculated the volume of excess feeder cells to remove. Removed excess feeder cells.

Using a 1.0 mL syringe and 16G needle, drew up 0.15 mL of OKT3 and added OKT3. Heat sealed the feeder cell suspension transfer pack.

Day 11 G-REX Fill and Seed Set up G-REX500MCS. Removed “Complete CM2 Day 11 Media”, from incubator and pumped media into G-REX500MCS. Pumped 4.5 L of media into the G-REX500MCS, filling to the line marked on the flask. Heat sealed and incubated flask as needed. Welded the Feeder Cell suspension transfer pack to the G-REX500MCS. Added Feeder Cells to G-REX500MCS. Heat sealed. Welded the TIL Suspension transfer pack to the flask. Added TIL to G-REX500MCS. Heat sealed. Incubated G-REX500MCS at 37.0±2.0° C., CO2 Percentage: 5.0±1.5% CO2.

Calculated incubation window. Performed calculations to determine the proper time to remove G-REX500MCS from incubator on Day 16. Lower limit: Time of incubation+108 hours. Upper limit: Time of incubation+132 hours.

Day 11 Excess TIL Cryopreservation. Applicable: Froze Excess TIL Vials. Verified the CRF has been set up prior to freeze. Perform Cryopreservation. Transferred vials from Controlled Rate Freezer to the appropriate storage. Upon completion of freeze, transfer vials from CRF to the appropriate storage container. Transferred vials to appropriate storage. Recorded storage location in LN2.

Day 16 Media Preparation. Pre-warmed AIM-V Media. Calculated time Media was warmed for media bags 1, 2, and 3. Ensured all bags have been warmed for a duration between 12 and 24 hours. Setup 10 L Labtainer for Supernatant. Attached the larger diameter end of a fluid pump transfer set to one of the female ports of a 10 L Labtainer bag using the Luer connectors. Setup 10 L Labtainer for Supernatant and label. Setup 10 L Labtainer for Supernatant. Ensure all clamps were closed prior to removing from the BSC. NOTE: Supernatant bag was used during TIL Harvest, which may be performed concurrently with media preparation.

Thawed IL-2. Thawed 5×1.1 mL aliquots of IL-2 (6×10⁶ IU/mL) (BR71424) per bag of CTS AIM V media until all ice had melted. Aliquoted 100.0 mL GlutaMax™. Added IL-2 to GlutaMax™. Prepared CTS AIM V media bag for formulation. Prepared CTS AIM V media bag for formulation. Stage Baxa Pump. Prepared to formulate media. Pumped GlutaMax™ +IL-2 into bag. Monitored parameters: Temperature LED Display: 37.0±2.0° C., CO₂ Percentage: 5.0±1.5% CO₂. Warmed Complete CM4 Day 16 Media. Prepared Dilutions.

Day 16 REP Spilt. Monitored Incubator parameters: Temperature LED display: 37.0±2.0° C., CO₂ Percentage: 5.0±1.5% CO₂. Removed G-REX500MCS from the incubator. Prepared and labeled 1 L Transfer Pack as TIL Suspension and weighed 1 L.

Volume Reduction of G-REX500MCS. Transferred ˜4.5 L of culture supernatant from the G-REX500MCS to the 10 L Labtainer.

Prepared flask for TIL harvest. After removal of the supernatant, closed all clamps to the red line.

Initiation of TIL Harvest. Vigorously tap flask and swirl media to release cells and ensure all cells have detached.

TIL Harvest. Released all clamps leading to the TIL suspension transfer pack. Using the GatheRex transferred the cell suspension into the TIL Suspension transfer pack. NOTE: Be sure to maintain the tilted edge until all cells and media are collected. Inspected membrane for adherent cells. Rinsed flask membrane. Closed clamps on G-REX500MCS. Heat sealed the Transfer Pack containing the TIL. Heat sealed the 10 L Labtainer containing the supernatant. Recorded weight of Transfer Pack with cell suspension and calculate the volume suspension. Prepared transfer pack for sample removal. Removed testing samples from cell supernatant.

Sterility & BacT testing sampling. Removed a 1.0 mL sample from the 15 mL conical labeled BacT prepared. Removed Cell Count Samples. In the BSC, using separate 3 mL syringes for each sample, removed 4×1.0 mL cell count samples from “TIL Suspension” transfer pack.

Removed Mycoplasma samples. Using a 3 mL syringe, removed 1.0 mL from TIL Suspension transfer pack and place into 15 mL conical labeled “Mycoplasma diluent” prepared.

Prepared transfer pack for seeding. Placed TIL in incubator. Removed cell suspension from the BSC and place in incubator until needed. Performed cell counts and calculations. Diluted cell count samples initially by adding 0.5 mL of cell suspension into 4.5 mL of AIM-V media prepared which gave a 1:10 dilution. Determined the average of viable cell concentration and viability of the cell counts performed. Determined upper and lower limit for counts. Note: dilution may be adjusted according based off the expected concentration of cells. Determined an average viable cell concentration from all four counts performed. Adjusted volume of TIL suspension. Calculated the adjusted volume of TIL suspension after removal of cell count samples. Total TIL cell volume minus 5.0 mL removed for testing.

Calculated total viable TIL cells. Calculated the total number of flasks to seed. NOTE: The maximum number of G-REX500MCS flasks to seed was five. If the calculated number of flasks to seed exceeded five, only five were seeded using the entire volume of cell suspension available.

Calculate number of flasks for subculture. Calculated the number of media bags required in addition to the bag prepared. Prepared one 10 L bag of “CM4 Day 16 Media” for every two G-REX-500M flask needed as calculated. Proceeded to seed the first GREX-500M flask(s) while additional media is prepared and warmed. Prepared and warmed the calculated number of additional media bags determined. Filled G-REX500MCS. Prepared to pump media and pumped 4.5 L of media into G-REX500MCS. Heat Sealed. Repeated Fill. Incubated flask. Calculated the target volume of TIL suspension to add to the new G-REX500MCS flasks. If the calculated number of flasks exceeds five only five will be seeded, USING THE ENTIRE VOLUME OF CELL SUSPENSION. Prepared Flasks for Seeding. Removed G-REX500MCS from the incubator. Prepared G-REX500MCS for pumping. Closed all clamps on except large filter line. Removed TIL from incubator. Prepared cell suspension for seeding. Sterile welded (per Process Note 5.11) “TIL Suspension” transfer pack to pump inlet line. Placed TIL suspension bag on a scale.

Seeded flask with TIL Suspension. Pump the volume of TIL suspension calculated into flask. Heat sealed. Filled remaining flasks.

Monitored Incubator. Incubator parameters: Temperature LED Display: 37.0±2.0° C., CO₂ Percentage: 5.0±1.5% CO₂. Incubated Flasks.

Determined the time range to remove G-REX500MCS from incubator on Day 22.

Day 22 Wash Buffer Preparation. Prepared 10 L Labtainer Bag. In BSC, attach a 4″ plasma transfer set to a 10 L Labtainer Bag via luer connection. Prepared 10 L Labtainer Bag. Closed all clamps before transferring out of the BSC. NOTE: Prepared one 10 L Labtainer Bag for every two G-REX500MCS flasks to be harvested. Pumped Plasmalyte into 3000 mL bag and removed air from 3000 mL Origen bag by reversing the pump and manipulating the position of the bag. Added human albumin 25% to 3000 mL Bag. Obtain a final volume of 120.0 mL of human albumin 25%.

Prepared IL-2 diluent. Using a 10 mL syringe, removed 5.0 mL of LOVO Wash Buffer using the needleless injection port on the LOVO Wash Buffer bag. Dispensed LOVO wash buffer into a 50 mL conical tube.

CRF blank bag LOVO wash buffer aliquoted. Using a 100 mL syringe, drew up 70.0 mL of LOVO Wash Buffer from the needleless injection port.

Thawed one 1.1 mL of IL-2 (6×10⁶ IU/mL), until all ice has melted. Added 50 μL IL-2 stock (6×10⁶ IU/mL) to the 50 mL conical tube labeled “IL-2 Diluent.”

Cryopreservation preparation. Placed 5 cryo-cassettes at 2-8° C. to precondition them for final product cryopreservation.

Prepared cell count dilutions. In the BSC, added 4.5 mL of AIM-V Media that has been labelled with lot number and “For Cell Count Dilutions” to 4 separate 15 mL conical tubes. Prepared cell counts. Labeled 4 cryovials with vial number (1-4). Kept vials under BSC to be used.

Day 22 TIL Harvest. Monitored Incubator. Incubator Parameters Temperature LED display: 37±2.0° C., CO2 Percentage: 5%±1.5%. Removed G-REX500MCS Flasks from Incubator. Prepared TIL collection bag and labeled. Sealed off extra connections. Volume Reduction: Transferred ˜4.5 L of supernatant from the G-REX500MCS to the Supernatant bag.

Prepared flask for TIL harvest. Initiated collection of TIL. Vigorously tap flask and swirl media to release cells. Ensure all cells have detached. Initiated collection of TIL. Released all clamps leading to the TIL suspension collection bag. TIL Harvest. Using the GatheRex, transferred the TIL suspension into the 3000 mL collection bag. Inspect membrane for adherent cells. Rinsed flask membrane. Closed clamps on G-Rex500MCS and ensured all clamps are closed. Transferred cell suspension into LOVO source bag. Closed all clamps. Heat Sealed. Removed 4×1.0 mL Cell Counts Samples

Performed Cell Counts. Performed cell counts and calculations utilizing NC-200 and Process Note 5.14. Diluted cell count samples initially by adding 0.5 mL of cell suspension into 4.5 mL of AIM-V media prepared. This gave a 1:10 dilution. Determined the average viability, viable cell concentration, and total nucleated cell concentration of the cell counts performed. Determined Upper and Lower Limit for counts. Determined the average viability, viable cell concentration, and total nucleated cell concentration of the cell counts performed. Weighed LOVO source bag. Calculated total viable TIL Cells. Calculated total nucleated cells.

Prepared Mycoplasma Diluent. Removed 10.0 mL from one supernatant bag via luer sample port and placed in a 15 mL conical.

Performed “TIL G-REX Harvest” protocol and determined the final product target volume. Loaded disposable kit. Removed filtrate bag. Entered Filtrate capacity. Placed Filtrate container on benchtop. Attached PlasmaLyte. Verified that the PlasmaLyte was attached and observed that the PlasmaLyte is moving. Attached Source container to tubing and verified Source container was attached. Confirmed PlasmaLyte was moving.

Final Formulation and Fill. Target volume/bag calculation. Calculated volume of CS-10 and LOVO wash buffer to formulate blank bag. Prepared CRF Blank.

Calculated the volume of IL-2 to add to the Final Product. Final IL-2 Concentration desired (IU/mL)—3001U/mL. IL-2 working stock: 6×10⁴ IU/mL. Assembled connect apparatus. Sterile welded a 4S-4M60 to a CC2 cell connection. Sterile welded the CS750 cryobags to the harness prepared. Welded CS-10 bags to spikes of the 4S-4M60. Prepared TIL with IL-2. Using an appropriately sized syringe, removed amount of IL-2 determined from the “IL-2 6×10⁴” aliquot. Labeled formulated TIL Bag. Added the formulated TIL bag to the apparatus. Added CS10. Switched Syringes. Drew −10 mL of air into a 100 mL syringe and replaced the 60 mL syringe on the apparatus. Added CS10. Prepared CS-750 bags. Dispensed cells.

Removed air from final product bags and take retain. Once the last final product bag was filled, closed all clamps. Drew 10 mL of air into anew 100 mL syringe and replace the syringe on the apparatus. Dispensed retain into a 50 mL conical tube and label tube as “Retain” and lot number. Repeat air removal step for each bag.

Prepared final product for cryopreservation, including visual inspection. Held the cryobags on cold pack or at 2-8° C. until cryopreservation.

Removed cell count sample. Using an appropriately sized pipette, remove 2.0 mL of retain and place in a 15 mL conical tube to be used for cell counts. Performed cell counts and calculations. NOTE: Diluted only one sample to appropriate dilution to verify dilution is sufficient. Diluted additional samples to appropriate dilution factor and proceed with counts. Determined the Average of Viable Cell Concentration and Viability of the cell counts performed. Determined Upper and Lower Limit for counts. NOTE: Dilution may be adjusted according based off the expected concentration of cells. Determined the Average of Viable Cell Concentration and Viability. Determined Upper and Lower Limit for counts. Calculated IFN-γ. Heat Sealed Final Product bags.

Labeled and collected samples per exemplary sample plan below.

TABLE 48
Sample plan.

| | | Sample | |
|---|---|---|---|
| | | Volume to | |
| | Number of | Add to | Container |
| Sample | Containers | Each | Type |
| *Mycoplasma | 1 | 1.0 mL | 15 mL Conical |
| Endotoxin | 2 | 1.0 mL | 2 mL Cryovial |
| Gram Stain | 1 | 1.0 mL | 2 mL Cryovial |
| IFN-γ | 1 | 1.0 mL | 2 mL Cryovial |
| Flow Cytometry | 1 | 1.0 mL | 2 mL Cryovial |
| **Bac-T Sterility | 2 | 1.0 mL | Bac-T Bottle |
| QC Retain | 4 | 1.0 mL | 2 mL Cryovial |
| Satellite Vials | 10 | 0.5 mL | 2 mL Cryovial |

Sterility and BacT testing. Testing Sampling. In the BSC, remove a 1.0 mL sample from the retained cell suspension collected using an appropriately sized syringe and inoculate the anaerobic bottle. Repeat the above for the aerobic bottle.

Final Product Cryopreservation. Prepared controlled rate freezer (CRF). Verified the CRF had been set up. Set up CRF probes. Placed final product and samples in CRF. Determined the time needed to reach 4° C.±1.5° C. and proceed with the CRF run. CRF completed and stored. Stopped the CRF after the completion of the run. Remove cassettes and vials from CRF. Transferred cassettes and vials to vapor phase LN2 for storage. Recorded storage location.

Post-Processing and analysis of final drug product included the following tests: (Day 22) Determination of CD3+ cells on Day 22 REP by flow cytometry; (Day 22) Gram staining method (GMP); (Day 22) Bacterial endotoxin test by Gel Clot LAL Assay (GMP); (Day 16) BacT Sterility Assay (GMP); (Day 16) Mycoplasma DNA detection by TD-PCR (GMP); Acceptable appearance attributes; (Day 22) BacT sterility assay (GMP)(Day 22); (Day 22) IFN-gamma assay. Other potency assay as described herein are also employed to analyze TIL products.

### Example 8: An Exemplary Embodiment of the Gen 3 Expansion Platform

### Day 0

Prepared tumor wash media. Media warmed prior to start. Added 5 mL of gentamicin (50 mg/mL) to the 500 mL bottle of HBSS. Added 5 mL of Tumor Wash Media to a 15 mL conical to be used for OKT3 dilution. Prepared feeder cell bags. Sterilely transferred feeder cells to feeder cell bags and stored at 37° C. until use or freeze. Counted feeder cells if at 37° C. Thawed and then counted feeder cells if frozen.

Optimal range for the feeder cell concentration is between 5×10⁴ and 5×10⁶ cells/mL. Prepared four conical tubes with 4.5 mL of AIM-V. Added 0.5 mL of cell fraction for each cell count. If total viable feeder cell number was ≥1×10⁹ cells, proceeded to adjust the feeder cell concentration. Calculated the volume of feeder cells to remove from the first feeder cell bag in order to add 1×10⁹ cells to a second feeder cell bag.

Using the p1000 micropipette, transferred 900 μL of Tumor Wash Media to the OKT3 aliquot (100 μL). Using a syringe and sterile technique, drew up 0.6 mL of OKT3 and added into the second feeder cell bag. Adjusted media volume to a total volume of 2 L. Transferred the second feeder cells bag to the incubator.

OKT3 formulation details: OKT3 may be aliquoted and frozen in original stock concentration from the vial (1 mg/mL) in 100 μL aliquots. ˜10× aliquots per 1 mL vial. Stored at −80 C. Day 0: 15 μg/flask, i.e. 30 ng/mL in 500 mL-60 μL max ˜1 aliquot.

Added 5 mL of Tumor Wash Medium into all wells of the 6-well plate labelled Excess Tumor Pieces. Kept the Tumor Wash Medium available for further use in keeping the tumor hydrated during dissection. Added 50 mL of Tumor Wash Medium to each 100 mm petri dish.

Dissected the tumor into 27 mm³ fragments (3×3×3 mm), using the ruler under the Dissection dish lid as a reference. Dissected intermediate fragment until 60 fragments were reached. Counted total number of final fragments and prepared G-REX-100MCS flasks according to the number of final fragments generated (generally 60 fragments per flask).

Retained favorable tissue fragments in the conical tubes labeled as Fragments Tube 1 through Fragments Tube 4. Calculated the number of G-REX-100MCS flasks to seed with feeder cell suspension according to the number of fragments tubes originated.

Removed feeder cells bag from the incubator and seed the G-REX-100MCS. Label as D0 (Day 0).

Tumor fragment addition to culture in G-REX-100 MCS. Under sterile conditions, unscrewed the cap of the G-REX-100MCS labelled Tumor Fragments Culture (D0) 1 and the 50 mL conical tube labelled Fragments Tube. Swirled the opened Fragments Tube 1 and, at the same time, slightly lifted the cap of the G-REX100MCS. Added the medium with the fragments to the G-REX100MCS while being swirled. Recorded the number of fragments transferred into the G-REX100MCS.

Once the fragments were located at the bottom of the GREX flask, drew 7 mL of media and created seven 1 mL aliquots-5 mL for extended characterization and 2 mL for sterility samples. Stored the 5 aliquots (final fragment culture supernatant) for extended characterization at −20° C. until needed.

Inoculated one anaerobic BacT/Alert bottle and one aerobic BacT/Alert bottle each with 1 mL of final fragment culture supernatant. Repeat for each flask sampled.

### At Day 7-8

Prepared feeder cell bags. Thawed feeder bags for 3-5 minutes in 37° C. water bath when frozen. Counted feeder cells if frozen. Optimal range for the feeder cell concentration is between 5×10⁴ and 5×10⁶ cells/mL. Prepared four conical tubes with 4.5 mL of AIM-V. Added 0.5 mL of cell fraction for each cell count into a new cryovial tube. Mixed the samples well and proceeded with the cell count.

If total viable feeder cell number was ≥2×10⁹ cells, proceeded to the next step to adjust the feeder cell concentration. Calculated the volume of feeder cells to remove from the first feeder cell bag in order to add 2×10⁹ cells to the second feeder cell bag.

Using the p1000 micropipette, transfer 900 μL of HBSS to a 100 μL OKT3 aliquot. Mix by pipetting up and down 3 times. Prepared two aliquots.

OKT3 formulation details: OKT3 may be aliquoted and frozen in original stock concentration from the vial (1 mg/mL) in 100 μL aliquots. ˜10× aliquots per 1 mL vial. Stored at −80 C. Day 7/8: 30 μg/flask, i.e. 60 ng/mL in 500 mL-120 μl max ˜2 aliquots.

Using a syringe and sterile technique, drew up 0.6 mL of OKT3 and added into the feeder cell bag, ensuring all added. Adjusted media volume to a total volume of 2 L. Repeated with second OKT3 aliquot and added to the feeder cell bag. Transferred the second feeder cells bag to the incubator.

Preparation of G-REX100MCS flask with feeder cell suspension. Recorded the number of G-REX-100MCS flasks to process according to the number of G-REX flasks generated on Day 0. Removed G-REX flask from incubator and removed second feeder cells bag from incubator.

Removal of supernatant prior to feeder cell suspension addition. Connected one 10 mL syringe to the G-REX100 flask and drew up 5 mL of media. Created five 1 mL aliquots-5 mL for extended characterization and stored the 5 aliquots (final fragment culture supernatant) for extended characterization at −20° C. until requested by sponsor.

Labeled and repeated for each G-REX100 flask.

5-20×1 mL samples for characterization, depending on number of flasks:
5 mL=1 flask
10 mL=2 flasks
15 mL=3 flasks
20 mL=4 flasks

Continued seeding feeder cells into the G-REX100 MCS and repeated for each G-REX100 MCS flask. Using sterile transfer methods, gravity transferred 500 mL of the second feeder cells bag by weight (assume 1 g=1 mL) into each G-REX-100MCS flask and recorded amount. Labeled as Day 7 culture and repeated for each G-REX100 flask. Transferred G-REX-100MCS flasks to the incubator.

### Day 10-11

Removed the first G-REX-100MCS flask and using sterile conditions removed 7 mL of pre-process culture supernatant using a 10 mL syringe. Created seven 1 mL aliquots −5 mL for extended characterization and 2 mL for sterility samples.

Mixed the flask carefully and using a new 10 mL syringe remove 10 mL supernatant and transfer to a 15 mL tube labelled as D10/11 Mycoplasma supernatant.

Mixed the flask carefully and using a new syringe removed the volume below according to how many flasks were to be processed:

$$\begin{matrix}1 flask = 40 mL \\ 2 flask = 20 mL / flask \\ 3 flask = 13.3 mL / flask \\ 4 flask = 10 mL / flask\end{matrix}$$

A total of 40 mL should be pulled from all flasks and pooled in a 50 mL conical tube labeled ‘Day 10/11 QC Sample’ and stored in the incubator until needed. Performed a cell count and allocated the cells.

Stored the 5 aliquots (pre-process culture supernatant) for extended characterization at ≤−20° C. until needed. Inoculated one anaerobic BacT/Alert bottle and one aerobic BacT/Alert bottle each with 1 mL of pre-process culture supernatant.

Continued with cell suspension transferred to the G-REX-500MCS and repeated for each G-REX-100MCS. Using sterile conditions, transferred the contents of each G-REX-100MCS into a G-REX-500MCS, monitoring about 100 mL of fluid transfer at a time. Stopped transfer when the volume of the G-REX-100MCS was reduced to 500 mL.

During transfer step, used 10 mL syringe and drew 10 mL of cell suspension into the syringe from the G-REX-100MCS. Followed the instructions according to the number of flasks in culture. If only 1 flask: Removed 20 mL total using two syringes. If 2 flasks: removed 10 mL per flask. If 3 flasks: removed 7 mL per flask. If 4 flasks: removed 5 mL per flask. Transferred the cell suspension to one common 50 mL conical tube. Keep in the incubator until the cell count step and QC sample. Total number of cells needed for QC was ˜20e6 cells: 4×0.5 mL cell counts (cell counts were undiluted first).

The quantities of cells needed for assays are as follows:
1. 10×10⁶ cells minimum for potency assays, such as those described herein, or for an IFN-γ or granzyme B assay
2. 1×10⁶ cells for Mycoplasma
3. 5×10⁶ cells for flow cytometry for CD3+/CD45+

Transferred the G-REX-500MCS flasks to the incubator.

Prepared QC Samples. At least 15×10⁸ cells were needed for the assays in this embodiment. Assays included: Cell count and viability; Mycoplasma (1×10⁶ cells/average viable concentration;) flow (5×10⁶ cells/average viable concentration;) and IFN-g assay (5×10⁶ cells-1×10⁶ cells; 8-10×10⁶ cells are required for the IFN-γ assay.

Calculated the volume of cells fraction for cryopreservation at 10×10⁶ cells/mL and calculated the number of vials to prepare

### Day 16-17

Wash Buffer preparation (1% HSA Plasmalyte A). Transferred HSA and Plasmalyte to 5 L bag to make LOVO wash buffer. Using sterile conditions, transferred a total volume of 125 mL of 25% HSA to the 5 L bag. Removed and transferred 10 mL or 40 mL of wash buffer in the ‘IL-2 6×10⁴ IU/mL’ tube (10 mL if IL-2 was prepared in advance or 40 mL if IL-2 was prepared fresh).

Calculated volume of reconstituted IL-2 to add to Plasmalyte+1% HSA: volume of reconstituted IL-2=(Final concentration of IL-2×Final volume)/specific activity of the IL-2 (based on standard assay). The Final Concentration of IL-2 was 6×10⁴ IU/mL.

The final volume was 40 mL.

Removed calculated initial volume of IL-2 needed of reconstituted IL-2 and transfer to the ‘IL-2 6×10⁴ IU/mL’ tube. Added 100 μL of IL-2 6×10⁶ IU/mL from the aliquot prepared in advance to the tube labelled ‘IL-2 6×10⁴ IU/mL’ containing 10 mL of LOVO wash buffer.

Removed about 4500 mL of supernatant from the G-REX-500MCS flasks. Swirled the remaining supernatant and transferred cells to the Cell Collection Pool bag. Repeated with all G-REX-500MCS flasks.

Removed 60 mL of supernatant and add to supernatant tubes for quality control assays, including Mycoplasma detection. Stored at +2-8° C.

Cell collection. Counted cells. Prepare four 15 mL conicals with 4.5 mL of AIM-V. These may be prepared in advance. Optimal range=is between 5×10⁴ and 5×10⁶ cells/mL. (1:10 dilution was recommended). For 1:10 dilution, to 4500 μL of AIM V prepared previously, add 500 μL of CF. Recorded dilution factor.

$$Calculated the TC ( Total Cells ) pre - LOVO ( live + dead ) =$$

$$\begin{matrix}Average Total Cell \\ Concentration ( TC conc pre LOVO ) \\ ( live + dead ) \\ X \\ Volume of Source bag\end{matrix}$$

$$Calculated the TVC ( Total Viable Cells ) pre - LOVO ( live ) =$$

$$\begin{matrix}Average Total Viable Cell \\ Concentration ( TVC pre LOVO ) \\ ( live ) \\ X \\ Volume of LOVO Source bag\end{matrix}$$

When the total cell (TC) number was >5×10, remove 5×10⁸ cells to be cryopreserved as MDA retention samples. 5×10⁸÷avg TC concentration (step 14.44)=volume to remove.

When the total cell (TC) number was ≤5×10⁹, remove 4×10⁶ cells to be cryopreserved as MDA retention samples. 4×10⁶÷avg TC concentration=volume to remove.

When the total cell number was determined, the number of cells to remove should allow retention of 150×10⁹ viable cells. Confirm TVC pre-LOVO 5×10⁸ or 4×10⁶ or not applicable. Calculated the volume of cells to remove.

Calculated the remaining Total Cells Remaining in Bag. Calculated the TC (Total Cells) pre-LOVO. [Avg. Total cell concentration X Remaining Volume=TC pre-LOVO Remaining]

According to the total number of cells remaining, the corresponding process in Table 49 is selected.

TABLE 49
Total number of cells.

| Total cells: | Retentate (mL) |
|---|---|
| 0 < Total cells ≤ 31 × 10⁹ | 115 |
| 31 × 10⁹ < Total cells ≤ 71 × 10⁹ | 165 |
| 71 × 10⁹ < Total Cells ≤ 110 × 10⁹ | 215 |
| 110 × 10⁹ < Total Cells ≤ 115 × 10⁹ | 265 |

Chose the volume of IL-2 to add corresponding to the used process. Volume calculated as: Retentate Volume×2×300 IU/mL=IU of IL-2 required. IU of IL-2 required/6×10⁴IU/mL=Volume of IL-2 to add Post LOVO bag. Recorded all volumes added. Obtained samples in cryovial for further analyses.

Mixed the cell product well. Sealed all bags for further processing, included cryopreservation when applicable.

Performed endotoxin, IFN-γ, sterility, and other assays as needed on cryovial samples obtained.

### Example 9: Gen 2 and Gen 3 Exemplary Processes

This example demonstrates the Gen 2 and Gen 3 processes. Process Gen 2 and Gen 3 TILs are generally composed of autologous TIL derived from an individual patient through surgical resection of a tumor and then expanded ex vivo. The priming first expansion step of the Gen 3 process was a cell culture in the presence of interleukin-2 (IL-2) and the monoclonal antibody OKT3, which targets the T-cell co-receptor CD3 on a scaffold of irradiated peripheral blood mononuclear cells (PBMCs).

The manufacture of Gen 2 TIL products consists of two phases: 1) pre-Rapid Expansion (Pre-REP) and 2) Rapid Expansion Protocol (REP). During the Pre-REP resected tumors were cut up into ≤50 fragments 2-3 mm in each dimension which were cultured with serum-containing culture medium (RPMI 1640 media containing 10% HuSAB supplemented) and 6,000 IU/mL of Interleukin-2 (IL-2) for a period of 11 days. On day 11 TIL were harvested and introduced into the large-scale secondary REP expansion. The REP consists of activation of ≤200×10⁶ of the viable cells from pre-REP in a co-culture of 5×10⁹ irradiated allogeneic PBMCs feeder cells loaded with 150 μg of monoclonal anti-CD3 antibody (OKT3) in a 5 L volume of CM2 supplemented with 3000 IU/mL of rhIL-2 for 5 days. On day 16 the culture is volume reduced 90% and the cell fraction is split into multiple G-REX-500 flasks at ≥1×10⁹ viable lymphocytes/flask and QS to 5 L with CM4. TIL are incubated an additional 6 days. The REP is harvested on day 22, washed, formulated, and cryo-preserved prior to shipping at −150° C. to the clinical site for infusion.

The manufacture of Gen 3 TIL products consists of three phases: 1) Priming First Expansion Protocol, 2) Rapid Second Expansion Protocol (also referred to as rapid expansion phase or REP), and 3) Subculture Split. To effect the Priming First Expansion TIL propagation, resected tumor was cut up into ≤120 fragments 2-3 mm in each dimension. On day 0 of the Priming First Expansion, a feeder layer of approximately 2.5×10⁸ allogeneic irradiated PBMCs feeder cells loaded with OKT-3 was established on a surface area of approximately 100 cm² in each of 3 100 MCS vessels. The tumor fragments were distributed among and cultured in the 3 100 MCS vessels each with 500 mL serum-containing CM1 culture medium and 6,000 IU/mL of Interleukin-2 (IL-2) and 15 ug OKT-3 for a period of 7 days. On day 7, REP was initiated by incorporating an additional feeder cell layer of approximately 5×10⁸ allogeneic irradiated PBMCs feeder cells loaded with OKT-3 into the tumor fragmented culture phase in each of the three 100 MCS vessels and culturing with 500 mL CM2 culture medium and 6,000 IU/mL IL-2 and 30 μg OKT-3. The REP initiation was enhanced by activating the entire Priming First Expansion culture in the same vessel using closed system fluid transfer of OKT3 loaded feeder cells into the 100MCS vessel. For Gen 3, the TIL scale up or split involved process steps where the whole cell culture was scaled to a larger vessel through closed system fluid transfer and was transferred (from 100 M flask to a 500 M flask) and additional 4 L of CM4 media was added. The REP cells were harvested on day 16, washed, formulated, and cryo-preserved prior to shipping at −150° C. to the clinical site for infusion.

Overall, the Gen 3 process is a shorter, more scalable, and easily modifiable expansion platform that will accommodate to fit robust manufacturing and process comparability.

TABLE 50
Comparison of Exemplary Gen 2 and Exemplary Gen 3 manufacturing process.

| Step | Process (Gen 2) | Process (Gen 3) |
|---|---|---|
| Pre REP- | Up to 50 fragments, 1 G-REX- | Whole tumor up to 120 fragments divided |
| day 0 | 100MCS, 11 days | evenly among up to 3 flasks. 1 flask: |
| | In 1 L of CM1 media + | 1-60 fragments |
| | IL-2 (6000 IU/mL) | 2 flasks: 61-89 fragments |
| | | 3 flasks 90-120 fragments |
| | | 7 days in 500 mL of CM1 media + |
| | | IL-2 (6000 IU/mL) |
| | | 2.5 × 10⁸ feeder cells/flask |
| | | 15 ug OKT-3/flask |
| REP | Direct to REP, Day 11, | Direct to REP, Day 7, all cells, |
| Initiation | <200 × 10⁶ TIL | same G-REX-100MCS |
| | (1)G-REX-500MCS in 5 L CM2 | Add 500 CM2 media |
| | media | IL-2 (6000 IU/mL) |
| | IL-2 (3000 IU/mL) | 5 × 10⁸ feeder cells/flask |
| | 5 × 10⁹ feeder cells | 30 ug OKT-3/flask |
| | 150 ug OKT-3 | |
| TIL | Volume reduce and split cell fraction | Each G-REX-100MCS(1L) transfers |
| propagation | in up to 5 G-REX-500MCS | to 1 G-REX-500MCS |
| or Scale up | 4.5 L CM4 media + IL-2 | Add 4 L CM4 media + IL-2 |
| | (3000 IU/mL) | (3000 IU/mL) |
| | ≥1 × 10⁹ TVC/flask | Scale up on day 9 to 11 |
| | Split day 16 | |
| Harvest | Harvest day 22, | Harvest day 16 |
| | LOVO-automated cell washer | LOVO-automated cell washer |
| Final | Cryopreserved Product | Cryopreserved product |
| formulation | 300 IU/mL IL2- CS10 in LN₂, | 300 IU/mL IL-2-CS10 in LN₂, |
| | multiple aliquots | multiple aliquots |
| Process | 22 days | 16 days |
| time | | |

On day 0, for both processes, the tumor was washed 3 times and the fragments were randomized and divided into two pools; one pool per process. For the Gen 2 Process, the fragments were transferred to one −GREX 100MCS flask with 1 L of CM1 media containing 6,000 IU/mL rhIL-2. For the Gen 3 Process, fragments were transferred to one G-REX-100MCS flask with 500 mL of CM1 containing 6,000 IU/mL rhIL-2, 15 ug OKT-3 and 2.5×10⁸ feeder cells. Seeding of TIL for Rep initiation day occurred on different days according to each process. For the Gen 2 Process, in which the G-REX-100MCS flask was 90% volume reduced, collected cell suspension was transferred to a new G-REX-500MCS to start REP initiation on day 11 in CM2 media containing IL-2 (3000 IU/mL), plus 5×10⁹ feeder cells and OKT-3 (30 ng/mL). Cells were expanded and split on day 16 into multiple G-REX-500 MCS flasks with CM4 media with IL-2 (3000 IU/mL) per protocol. The culture was then harvested and cryopreserved on day 22 per protocol. For the Gen 3 process, the REP initiation occurred on day 7, in which the same G-REX-100MCS used for REP initiation. Briefly, 500 mL of CM2 media containing IL-2 (6000 IU/mL) and 5×10⁸ feeder cells with 30 ug OKT-3 was added to each flask. On day 9-11 the culture was scaled up. The entire volume of the G-REX100M (1 L) was transferred to a G-REX-500MCS and 4 L of CM4 containing IL-2 (3000 IU/mL) was added. Flasks were incubated 5 days. Cultures were harvested and cryopreserved on Day 16.

Three different tumors were included in the comparison, two lung tumors (L4054 and L4055) and one melanoma tumor (M1085T).

CM1 (culture media 1), CM2 (culture media 2), and CM4 (culture media 4) media were prepared in advance and held at 4° C. for L4054 and L4055. CM1 and CM2 media were prepared without filtration to compare cell growth with and without filtration of media.

Media was warmed at 37° C. up to 24 hours in advance for L4055 tumor on REP initiation and scale-up.

Results. Gen 3 results fell within 30% of Gen 2 for total viable cells achieved. Gen 3 final product exhibited higher production of IFN-γ after restimulation. Gen 3 final product exhibited increased clonal diversity as measured by total unique CDR3 sequences present. Gen 3 final product exhibited longer mean telomere length.

Pre-REP and REP expansion on Gen 2 and Gen 3 processes followed the procedures described above. For each tumor, the two pools contained equal number of fragments. Due to the small size of tumors, the maximum number of fragments per flask was not achieved. Total pre-REP cells (TVC) were harvested and counted at day 11 for the Gen 2 process and at day 7 for the Gen 3 process. To compare the two pre-REP arms, the cell count was divided over the number of fragments provided in the culture in order to calculate an average of viable cells per fragment. As indicated in Table 51 below, the Gen 2 process consistently grew more cells per fragment compared to the Gen 3 Process. An extrapolated calculation of the number of TVC expected for Gen 3 process at day 11, which was calculated dividing the pre-REP TVC by 7 and then multiply by 11.

TABLE 51
Pre-REP cell counts

| | Tumor ID | | | | | |
|---|---|---|---|---|---|---|
| | L4054 | | L4055* | | M1085T | |
| Process | Gen 2 | Gen 3 | Gen 2 | Gen 3 | Gen 2 | Gen 3 |
| pre-REP TVC | 1.42E+08 | 4.32E+07 | 2.68E+07 | 1.38E+07 | 1.23E+07 | 3.50E+06 |
| Number of fragments | 21 | 21 | 24 | 24 | 16 | 16 |
| Average TVC per fragment | 6.65E+06 | 2.06E+06 | 1.12E+06 | 5.75E+05 | 7.66E+05 | 2.18E+05 |
| at pre-REP | | | | | | |
| Gen 3 extrapolated value at | N/A | 6.79E+07 | N/A | 2.17E+07 | N/A | 5.49E+06 |
| pre REP day 11 | | | | | | |

*L4055, unfiltered media.

For the Gen 2 and Gen 3 processes, TVC was counted per process condition and percent viable cells was generated for each day of the process. On harvest, day 22 (Gen 2) and day 16 (Gen 3) cells were collected and the TVC count was established. The TVC was then divided by the number of fragments provided on day 0, to calculate an average of viable cells per fragment. Fold expansion was calculated by dividing harvest TVC by over the REP initiation TVC. As exhibited in Table 52, comparing Gen 2 and the Gen 3, fold expansions were similar for L4054; in the case of L4055, the fold expansion was higher for the Gen 2 process. Specifically, in this case, the media was warmed up 24 in advance of REP initiation day. A higher fold expansion was also observed in Gen 3 for M1085T. An extrapolated calculation of the number of TVC expected for Gen 3 process at day 22, which was calculated dividing the REP TVC by 16 and then multiply by 22.

TABLE 52
Total viable cell count and fold expansion on TIL final product.

| | Tumor ID | | | | | |
|---|---|---|---|---|---|---|
| | L4054 | | L4055 | | M1085T | |
| Process | Gen 2 | Gen 3 | Gen 2 | Gen 3 | Gen 2 | Gen 3 |
| # Fragments | 21 | 21 | 24 | 24 | 16 | 16 |
| TVC /fragment (at | 3.18E+09 | 8.77E+08 | 2.30E+09 | 3.65E+08 | 7.09E+08 | 4.80E+08 |
| Harvest) | | | | | | |
| REP initiation | 1.42E+08 | 4.32E+07 | 2.68E+07 | 1.38E+07 | 1.23E+07 | 3.50E+06 |
| Scale up | 3.36E+09 | 9.35E+08 | 3.49E+09 | 8.44E+08 | 1.99E+09 | 3.25E+08 |
| Harvest | 6.67E+10 | 1.84E+10 | 5.52E+10 | 8.76E+09 | 1.13E+10 | 7.68E+09 |
| Fold Expansion Harvest/ | 468.4 | 425.9 | 2056.8 | 634.6 | 925.0 | 2197.2 |
| REP initiation | | | | | | |
| Gen 3 extrapolated value at | N/A | 2.53E+10 | N/A | 1.20E+10 | N/A | 1.06E+10 |
| REP harvest day 22 | | | | | | |

* L4055, unfiltered media.

Table 53: % Viability of TIL final product: Upon harvest, the final TIL REP products were compared against release criteria for % viability. All of the conditions for the Gen 2 and Gen 3 processes surpassed the 70% viability criterion and were comparable across processes and tumors.

Upon harvest, the final TIL REP products were compared against release criteria for % viability. All of the conditions for the Gen 2 and Gen 3 processes surpassed the 70% viability criterion and were comparable across processes and tumors.

TABLE 53
% Viability of REP (TIL Final Product)

| | Tumor ID | | | | | |
|---|---|---|---|---|---|---|
| | L4054 | | L4055 | | M1085T | |
| Process | Gen 2 | Gen 3 | Gen 2 | Gen 3 | Gen 2 | Gen 3 |
| REP initiation | 98.23% | 97.97% | 97.43% | 92.03% | 81.85% | 68.27% |
| Scale up | 94.00% | 93.57% | 90.50% | 95.93% | 78.55% | 71.15% |
| Harvest | 87.95% | 89.85% | 87.50% | 86.70% | 86.10% | 87.45% |

Due to the number of fragments per flask below the maximum required number, an estimated cell count at harvest day was calculated for each tumor. The estimation was based on the expectation that clinical tumors were large enough to seed 2 or 3 flasks on day 0.

TABLE 54
Extrapolated estimate cell count calculation
to full scale 2 and 3 flask on Gen 3 Process.

| | Tumor ID | | | | | |
|---|---|---|---|---|---|---|
| | L4054 | | L4055 | | M1085T | |
| Gen 3 Process | 2 flasks | 3 Flasks | 2 flasks | 3 Flasks | 2 flasks | 3 Flasks |
| Estimate Harvest | 3.68E+10 | 5.52E+10 | 1.75E+10 | 2.63E+10 | 1.54E+10 | 2.30E+10 |

Immunophenotyping—phenotypic marker comparisons on TIL final product. Three tumors L4054, L4055, and M1085T underwent TIL expansion in both the Gen 2 and Gen 3 processes. Upon harvest, the REP TIL final products were subjected to flow cytometry analysis to test purity, differentiation, and memory markers. For all the conditions the percentage of TCR a/b+ cells was over 90%.

TIL harvested from the Gen 3 process showed a higher expression of CD8 and CD28 compared to TIL harvested from the Gen 2 process. The Gen 2 process showed a higher percentage of CD4+.

TIL harvested from the Gen 3 process showed a higher expression on central memory compartments compared to TIL from the Gen 2 process.

Activation and exhaustion markers were analyzed in TIL from two, tumors L4054 and L4055 to compare the final TIL product by from the Gen 2 and Gen 3 TIL expansion processes. Activation and exhaustion markers were comparable between the Gen 2 and Gen 3 processes.

Interferon gamma secretion upon restimulation. On harvest day, day 22 for Gen 2 and day 16 for Gen 3, TIL underwent an overnight restimulation with coated anti-CD3 plates for L4054 and L4055. The restimulation on M1085T was performed using anti-CD3, CD28, and CD137 beads. Supernatant was collected after 24 hours of the restimulation in all conditions and the supernatant was frozen. IFNγ analysis by ELISA was assessed on the supernatant from both processes at the same time using the same ELISA plate. Higher production of IFNγ from the Gen 3 process was observed in the three tumors analyzed.

Measurement of IL-2 levels in culture media. To compare the IL-2 consumption between Gen 2 and Gen 3 process, cell supernatant was collected on REP initiation, scale up, and harvest day, on tumor L4054 and L4055. The quantity of IL-2 in cell culture supernatant was measured by Quantitate ELISA Kit from R&D. The general trend indicates that the IL-2 concentration remains higher in the Gen 3 process when compared to the Gen 2 process. This is likely due to the higher concentration of IL-2 on REP initiation (6000 IU/mL) for Gen 3 coupled with the carryover of the media throughout the process.

Metabolic substrate and metabolite analysis. The levels of metabolic substrates such as D-glucose and L-glutamine were measured as surrogates of overall media consumption. Their reciprocal metabolites, such lactic acid and ammonia, were measured. Glucose is a simple sugar in media that is utilized by mitochondria to produce energy in the form of ATP. When glucose is oxidized, lactic acid is produced (lactate is an ester of lactic acid). Lactate is strongly produced during the cells exponential growth phase. High levels of lactate have a negative impact on cell culture processes.

Spent media for L4054 and L4055 was collected at REP initiation, scale up, and harvest days for both process Gen 2 and Gen 3. The spent media collection was for Gen 2 on Day 11, day 16 and day 22; for Gen 3 was on day 7, day 11 and day 16. Supernatant was analyzed on a CEDEX Bio-analyzer for concentrations of glucose, lactic acid, glutamine, GlutaMax™, and ammonia.

L-glutamine is an unstable essential amino acid required in cell culture media formulations. Glutamine contains an amine, and this amide structural group can transport and deliver nitrogen to cells. When L-glutamine oxidizes, a toxic ammonia by-product is produced by the cell. To counteract the degradation of L-glutamine the media for the Gen 2 and Gen 3 processes was supplemented with GlutaMax™, which is more stable in aqueous solutions and does not spontaneously degrade. In the two tumor lines, the Gen 3 arm showed a decrease in L-glutamine and GlutaMax™ during the process and an increase in ammonia throughout the REP. In the Gen 2 arm a constant concentration of L-glutamine and GlutaMax™, and a slight increase in the ammonia production was observed. The Gen 2 and Gen 3 processes were comparable at harvest day for ammonia and showed a slight difference in L-glutamine degradation.

Telomere repeats by Flow-FISH. Flow-FISH technology was used to measure the average length of the telomere repeat on L4054 and L4055 under Gen 2 and Gen 3 process. The determination of a relative telomere length (RTL) was calculated using Telomere PNA kit/FITC for flow cytometry analysis from DAKO. Gen 3 showed comparable telomere length to Gen 2.

CD3 Analysis. To determine the clonal diversity of the cell products generated in each process, TIL final product harvested for L4054 and L4055, were sampled and assayed for clonal diversity analysis through sequencing of the CDR3 portion of the T-cell receptors.

Table 55 shows a comparison between Gen 2 and Gen 3 of percentage shared unique CDR3 sequences on L4054 on TIL harvested cell product. 199 sequences are shared between Gen 3 and Gen 2 final product, corresponding to 97.07% of top 80% of unique CDR3 sequences from Gen 2 shared with Gen 3 final product.

TABLE 55
Comparison of shared uCDR3 sequences between
Gen 2 and Gen 3 processes on L4054.

| # uCDR3 | All uCDR3's | | Top 80% uCDR3's | |
|---|---|---|---|---|
| (% Overlap) | Gen 2 | Gen 3 | Gen 2 | Gen 3 |
| Gen 2-L4054 | 8915 | 4355 (48.85%) | 205 | 199 (97.07%) |
| Gen 3-L4054 | — | 18130 | — | 223 |

Table 56 shows a comparison between Gen 2 and Gen 3 of percentage shared unique CDR3 sequences on L4055 on TIL harvested cell product. 1833 sequences are shared between Gen 3 and Gen 2 final product, corresponding to 99.45% of top 80% of unique CDR3 sequences from Gen 2 shared with Gen 3 final product.

TABLE 56
Comparison of shared uCDR3 sequences between
Gen 2 and Gen 3 processes on L4055.

| # uCDR3 | All uCDR3's | | Top 80% uCDR3's | |
|---|---|---|---|---|
| (% Overlap) | Gen 2 | Gen 3 | Gen 2 | Gen 3 |
| Gen 2-L4055 | 12996 | 6599 (50.77%) | 1843 | 1833 (99.45%) |
| Gen 3-L4055 | — | 27246 | — | 2616 |

CM1 and CM2 media was prepared in advanced without filtration and held at 4 degree c. until use for tumor L4055 to use on Gen 2 and Gen 3 process.

Media was warmed up at 37 degree c. for 24 hours in advance for tumor L4055 on REP initiation day for Gen 2 and Gen 3 process.

LDH was not measured in the supernatants collected on the processes.

M1085T TIL cell count was executed with K2 cellometer cell counter.

On tumor M1085T, samples were not available such as supernatant for metabolic analysis, TIL product for activation and exhaustion markers analysis, telomere length and CD3−TCR vb Analysis.

Conclusions. This example compares 3 independent donor tumors tissue in terms of functional quality attributes, plus extended phenotypic characterization and media consumption among Gen 2 and Gen 3 processes.

Gen 2 and Gen 3 pre-REP and REP expansion comparison were evaluated in terms of total viable cells generated and viability of the total nucleated cell population. TVC cell doses at harvest day was not comparable between Gen 2 (22 days) and Gen 3 (16 days). Gen 3 cell doses were lower than Gen 2 at around 40% of total viable cells collected at harvest.

An extrapolated cell number was calculated for Gen 3 process assuming the pre-REP harvest occurred at day 11 instead day 7 and REP Harvest at Day 22 instead day 16. In both cases, Gen 3 shows a closer number on TVC compared to the Gen 2 process, indicating that the early activation enhanced TIL growth.

In the case of extrapolated value for extra flasks (2 or 3) on Gen 3 process assuming a bigger size of tumor processed, and reaching the maximum number of fragments required per process as described. It was observed that a similar dose can be reachable on TVC at Day 16 Harvest for Gen 3 process compared to Gen 2 process at Day 22. This observation is important and indicates an early activation of the culture reduced TIL processing time.

Gen 2 and Gen 3 pre-REP and REP expansion comparison were evaluated in terms of total viable cells generated and viability of the total nucleated cell population. TVC cell doses at harvest day was not comparable between Gen 2 (22 days) and Gen 3 (16 days). Gen 3 cell doses were lower than Gen 2 at around 40% of total viable cells collected at harvest.

In terms of phenotypic characterization, a higher CD8+ and CD28+ expression was observed on three tumors on Gen 3 process compared to Gen 2 process.

Gen 3 process showed slightly higher central memory compartments compared to Gen 2 process.

Gen 2 and Gen 3 process showed comparable activation and exhaustion markers, despite the shorter duration of the Gen 3 process.

IFN gamma (IFNγ) production was 3 times higher on Gen 3 final product compared to Gen 2 in the three tumors analyzed. This data indicates the Gen 3 process generated a highly functional and more potent TIL product as compared to the Gen 2 process, possibly due to the higher expression of CD8 and CD28 expression on Gen 3. Phenotypic characterization suggested positive trends in Gen 3 toward CD8+, CD28+ expression on three tumors compared to Gen 2 process.

Telomere length on TIL final product between Gen 2 and Gen 3 were comparable.

Glucose and Lactate levels were comparable between Gen 2 and Gen 3 final product, suggesting the levels of nutrients on the media of Gen 3 process were not affected due to the non-execution of volume reduction removal in each day of the process and less volume media overall in the process, compared to Gen 2.

Overall Gen 3 process showed a reduction almost two times of the processing time compared to Gen 2 process, which would yield a substantial reduction on the cost of goods (COGs) for TIL product expanded by the Gen 3 process.

IL-2 consumption indicates a general trend of IL-2 consumption on Gen 2 process, and in Gen 3 process IL-2 was higher due to the non-removal of the old media.

The Gen 3 process showed a higher clonal diversity measured by CDR3 TCRab sequence analysis.

The addition of feeders and OKT-3 on day 0 of the pre-REP allowed an early activation of TIL and allowed for TIL growth using the Gen 3 process.

Table 57 describes various embodiments and outcomes for the Gen 3 process as compared to the current Gen 2 process.

TABLE 57
Exemplary Gen 3 process features.

| Step | Process Gen 2 embodiment | Process Gen 3 embodiment |
|---|---|---|
| Pre REP- | ≤50 fragments | ≤240 fragments |
| day 0 | 1X G-REX-100MCS | ≤60 fragments/flask |
| | 1 L media | ≤4 flasks |
| | IL-2 (6000 IU/mL) | ≤2 L media (500 mL/flask) |
| | 11 days | IL-2 (6000 IU/mL) |
| | | 2.5 × 10⁸ feeder cells/flask |
| | | 15 ug OKT3/flask |
| REP | Fresh TIL direct to REP | Fresh TIL direct to REP |
| Initiation | Day 11 | Day 7 |
| | ≤200e⁶ viable cells | Activate entire culture |
| | 5 × 10⁹ feeder cells | 5 × 10⁸ feeder cells |
| | G-REX-500MCS | 30 μg OKT3/flask |
| | 5 L CM2 media + IL-2 | G-REX-100MCS |
| | (3000 IU/mL) | 500 mL media + IL-2 |
| | 150 μg OKT3 | (6000 IU/mL) |
| TIL Sub- | ≤5 G-REX-500MCS | ≤4 G-REX-500MCS |
| culture or | ≤1 × 10 viable | Scale up entire |
| Scale up | cells/flask | culture |
| | 5 L/flask | 4 L/flask |
| | Day 16 | Day 10-11 |
| Harvest | Harvest Day 22, | Harvest Day 16 |
| | LOVO-automated | LOVO-automated |
| | cell washer | cell washer |
| | 2 wash cycles | 5 wash cycles |
| Final | Cryopreserved Product | Cryopreserved product |
| formulation | 300 IU/mL IL2- | 300 IU/mL IL-2- |
| | CS10 in LN₂, | CS10 in LN₂, |
| | multiple aliquots | multiple aliquots |
| Process time | 22 days | 16 days |

### Example 10: An Exemplary Gen 3 Process (Also Referred to as Gen 3.1)

This example describes further studies regarding the “Comparability between the Gen 2 and Gen 3 processes for TIL expansion”. The Gen 3 process was modified to include an activation step early in the process with the goal of increasing the final total viable cell (TVC) output, while maintaining the phenotypic and functional profiles. As described below, a Gen 3 embodiment was modified as a further embodiment and is referred to herein in this example as Gen 3.1.

In some embodiments, the Gen 3.1 TIL manufacturing process has four operator interventions:
1. Tumor Fragment Isolation and Activation: On Day 0 of the process the tumor was dissected and the final fragments generated awe-3×3 mm each (up to 240 fragments total) and cultured in 1-4 G-REX100MCS flasks. Each flask contained up to 60 fragments, 500 mL of CM1 or DM1 media, and supplemented with 6,000 IU rhIL-2, 15 μg OKT3, and 2.5×10⁸ irradiated allogeneic mononuclear cells. The culture was incubated at 37° C. for 6-8 days.
2. TIL Culture Reactivation: On Day 7-8 the culture was supplemented through slow addition of CM2 or DM1 media supplemented with 6,000 IU rhIL-2, 30 μg OKT3, and 5×10⁸ irradiated allogeneic mononuclear cells in both cases. Care was taken to not disturb the existing cells at the bottom of the flask. The culture was incubated at 37° C. for 3-4 days.
3. Culture Scale Up: Occurs on day 10-11. During the culture scale-up, the entire contents of the G-REX100MCS was transferred to a G-REX500MCS flask containing 4 L of CM4 or DM2 supplemented with 3,000 IU/mL of IL-2 in both cases. Flasks were incubated at 37° C. for 5-6 days until harvest.
4. Harvest/Wash/Formulate: On day 16-17 the flasks are volume reduced and pooled. Cells were concentrated and washed with PlasmaLyte A pH 7.4 containing 1% HSA. The washed cell suspension was formulated at a 1:1 ratio with CryoStor10 and supplemented with rhIL-2 to a final concentration of 300 IU/mL.

The DP was cryopreserved with a controlled rate freeze and stored in vapor phase liquid nitrogen. *Complete Standard TIL media 1, 2, or 4 (CM1, CM2, CM4) could be substituted for CTS™OpTmizer™ T-Cell serum free expansion Medium, referred to as Defined Medium (DM1 or DM2), as noted above.

Process description. On day 0, the tumor was washed 3 times, then fragmented in 3×3×3 final fragments. Once the whole tumor was fragmented, then the final fragments were randomized equally and divided into three pools. One randomized fragment pool was introduced to each arm, adding the same number of fragments per the three experimental matrices.

Tumor L4063 expansion was performed with Standard Media and tumor L4064 expansion was performed with Defined Media (CTS OpTmizer) for the entire TIL expansion process. Components of the media are described herein.

CM1 Complete Media 1: RPMI+ Glutamine supplemented with 2 mM GlutaMax™, 10% Human AB Serum, Gentamicin (50 ug/mL), 2-Mercaptoethanol (55 uM). Final media formulation supplemented with 60001U/mL IL-2.

CM2 Complete Media 2: 50% CM1 medium+50% AIM-V medium. Final media formulation supplemented with 60001U/mL IL-2.

CM4 Complete Media 4: AIM-V supplemented with GlutaMax™ (2 mM) Final media formulation supplemented with 30001U/mL IL-2.

CTS OpTmizer CTS™OpTmizer™ T-Cell Expansion Basal Medium supplemented with CTS™ OpTmizer™ T-Cell Expansion Supplement (26 mL/L).

DM1: CTS™OpTmizer™ T-Cell Expansion Basal Medium supplemented with CTS™ OpTmizer™ T-Cell Expansion Supplement (26 mL/L), and CTS™ Immune Cell SR (3%), with GlutaMax™ (2 mM). Final formulation supplemented with 6,000 IU/mL of IL-2.

DM2: CTS™OpTmizer™ T-Cell Expansion Basal Medium supplemented with CTS™ OpTmizer™ T-Cell Expansion Supplement (26 mL/L), and CTS™ Immune Cell SR (3%), with GlutaMax™ (2 mM). Final formulation supplemented with 3,000 IU/mL of IL-2.

All types of media used, i.e., Complete (CM) and Defined (DM) media, were prepared in advance, held at 4° C. degree until the day before use, and warmed at 37° C. in an incubator for up to 24 hours in advance prior to process day.

TIL culture reactivation occurred on Day 7 for both tumors. Scale-up occurred on day 10 for L4063 and day 11 for L4064. Both cultures were harvested and cryopreserved on Day 16.

Results Achieved. Cells counted and % viability for Gen 3.0 and Gen 3.1 processes were determined. Expansion in all the conditions followed details described in this example.

For each tumor, the fragments were divided into three pools of equal numbers. Due to the small size of the tumors, the maximum number of fragments per flask was not achieved. For the three different processes, the total viable cells and cell viability were assessed for each condition. Cell counts were determined as TVC on day 7 for reactivation, TVC on day 10 (L4064) or day 11 (L4063) for scale-up, and TVC at harvest on day 16/17.

Cell counts for Day 7 and Day 10/11 were taken FIO. Fold expansion was calculated by dividing the harvest day 16/17 TVC by the day 7 reactivation day TVC. To compare the three arms, the TVC on harvest day was divided by the number of fragments added in the culture on Day 0 in order to calculate an average of viable cells per fragment.

Cell counts and viability assays were performed for L4063 and L4064. The Gen 3.1-Test process yielded more cells per fragment than the Gen 3.0 Process on both tumors.

Total viable cell count and fold expansion; % Viability during the process. On reactivation, scale up and harvest the percent viability was performed on all conditions. On day 16/17 harvest, the final TVC were compared against release criteria for % viability. All of the conditions assessed surpassed the 70% viability criterion and were comparable across processes and tumors.

Immunophenotyping—Phenotypic characterization on TIL final product. The final products were subjected to flow cytometry analysis to test purity, differentiation, and memory markers. Percent populations were consistent for TCRα/β, CD4+ and CD8+ cells for all conditions.

Extended phenotypic analysis of REP TIL was performed. TIL product showed a higher percentage of CD4+ cells for Gen 3.1 conditions compared to Gen 3.0 on both tumors, and higher percentage of CD28+ cells from CD8+ population for Gen 3.0 compared to Gen 3.1 conditions on both conditions.

TIL harvested from the Gen 3.0 and Gen 3.1 processes showed comparable phenotypic markers as CD27 and CD56 expression on CD4+ and CD8+ cells, and a comparable CD28 expression on CD4+ gated cells population. Memory markers comparison on TIL final product:

Frozen samples of TIL harvested on day 16 were stained for analysis. TIL memory status was comparable between Gen 3.0 and Gen 3.1 processes. Activation and exhaustion markers comparison on TIL final product:

Activation and exhaustion markers were comparable between the Gen 3.0 and Gen 3.1 processes gated on CD4+ and CD8+ cells.

Interferon gamma secretion upon restimulation. Harvested TIL underwent an overnight restimulation with coated anti-CD3 plates for L4063 and L4064. Higher production of IFNγ from the Gen 3.1 process was observed in the two tumors analyzed compared to Gen 3.0 process.

Measurement of IL-2 levels in culture media. To compare the levels of IL-2 consumption between all of the conditions and processes, cell supernatants were collected at initiation of reactivation on Day 7, at scale-up Day 10 (L4064)/11 (L4063), and at harvest Day 16/17, and frozen. The supernatants were subsequently thawed and then analyzed. The quantity of IL-2 in cell culture supernatant was measured by the manufacturer protocol.

Overall Gen 3 and Gen 3.1 processes were comparable in terms of IL-2 consumption during the complete process assessed across same media conditions. IL-2 concentration (pg/mL) analysis on spent media collected for L4063 and L4064.

Metabolite analysis. Spent media supernatants was collected from L4063 and L4064 at reactivation initiation on day 7, scale-up on day 10 (L4064) or day 11 (L4063), and at harvest on days 16/17 for L4063 and L4064, for every condition. Supernatants were analyzed on a CEDEX Bio-analyzer for concentrations of glucose, lactate, glutamine, GlutaMax™, and ammonia.

Defined media has a higher glucose concentration of 4.5 g/L compared to complete media (2 g/L). Overall, the concentration and consumption of glucose were comparable for Gen 3.0 and Gen 3.1 processes within each media type.

An increase in lactate was observed and increase in lactate was comparable between the Gen 3.0 and Gen 3.1 conditions and between the two media used for reactivation expansion (complete media and defined media).

In some instances, the standard basal media contained 2 mM L-glutamine and was supplemented with 2 mM GlutaMax™ to compensate for the natural degradation of L-glutamine in culture conditions to L-glutamate and ammonia.

In some instances, defined (serum free) media used did not contain L-glutamine on the basal media, and was supplemented only with GlutaMax™ to a final concentration of 2 mM. GlutaMax™ is a dipeptide of L-alanine and L-glutamine, is more stable than L-glutamine in aqueous solutions and does not spontaneously degrade into glutamate and ammonia. Instead, the dipeptide is gradually dissociated into the individual amino acids, thereby maintaining a lower but sufficient concentration of L-glutamine to sustain robust cell growth.

In some instances, the concentration of glutamine and GlutaMax™ slightly decreased on the scale-up day, but at harvest day showed an increase to similar or closer levels compared to reactivation day. For L4064, glutamine and GlutaMax™ concentration showed a slight degradation in a similar rate between different conditions, during the whole process.

Ammonia concentrations were higher samples grown in standard media containing 2 mM glutamine+2 mM GlutaMax™) than those grown in defined media containing 2 mM GlutaMax™). Further, as expected, there was a gradual increase or accumulation of ammonia over the course of the culture. There were no differences in ammonia concentrations across the three different test conditions.

Telomere repeats by Flow—FISH. Flow-FISH technology was used to measure the average length of the telomere repeat on L4063 and L4064 under Gen 3 and Gen 3.1 processes. The determination of a relative telomere length (RTL) was calculated using Telomere PNA kit/FITC for flow cytometry analysis from DAKO. Telomere assay was performed. Telomere length in samples were compared to a control cell line (1301 leukemia). The control cell line is a tetraploid cell line having long stable telomeres that allows calculation of a relative telomere length. Gen 3 and Gen 3.1 processes assessed in both tumors showed comparable telomere length.

TCR Vβ repertoire Analysis

To determine the clonal diversity of the cell products generated in each process, TIL final products were assayed for clonal diversity analysis through sequencing of the CDR3 portion of the T-cell receptors.

Three parameters were compared between the three conditions:
Diversity index of Unique CDR3 (uCDR3)
% shared uCDR3
For the top 80% of uCDR3:
   Compare the % shared uCDR3 copies
   Compare the frequency of unique clonotypes

Control and Gen 3.1 Test, percentage shared unique CDR3 sequences on TIL harvested cell product for: 975 sequences are shared between Gen 3 and Gen 3.1 Test final product, equivalent to 88% of top 80% of unique CDR3 sequences from Gen 3 shared with Gen 3.1.

Control and Gen 3.1 Test, percentage shared unique CDR3 sequences on TIL harvested cell product for: 2163 sequences are shared between Gen 3 and Gen 3.1 Test final product, equivalent to 87% of top 80% of unique CDR3 sequences from Gen 3 shared with Gen 3.1.

The number of unique CD3 sequences identified from 1×10⁶ cells collected on Harvest day 16, for the different processes. Gen 3.1 Test condition showed a slightly higher clonal diversity compared to Gen 3.0 based on the number of unique peptide CDRs within the sample.

The Shannon entropy diversity index is a reliable and common metric for comparison, because Gen 3.1 conditions on both tumors showed slightly higher diversity than Gen 3 process, suggesting that TCR Vβ repertoire for Gen 3.1 Test condition was more polyclonal than the Gen 3.0 process.

Additionally, the TCR Vβ repertoire for Gen 3.1 Test condition showed more than 87% overlap with the corresponding repertoire for Gen 3.0 process on both tumor L4063 and L4064.

The value of IL-2 concentration on spent media for Gen 3.1 Test L4064 on reactivation day was below to the expected value (similar to Gen 3.1 control and Gen 3.0 condition).

The low value could be due to a pipetting error, but because of the minimal sample taken it was not possible to repeat the assay.

Conclusions. Gen 3.1 test condition including feeders and OKT-3 on Day 0 showed a higher TVC of cell doses at Harvest day 16 compared to Gen 3.0 and Gen 3.1 control. TVC on the final product for Gen 3.1 test condition was around 2.5 times higher than Gen 3.0.

Gen 3.1 test condition with the addition of OKT-3 and feeders on day 0, for both tumor samples tested, reached a maximum capacity of the flask at harvest. Under these conditions, if a maximum of 4 flasks on day 0 is initiated, the final cell dose could be between 80-100×10⁹ TILs.

All the quality attributes such as phenotypic characterization including purity, exhaustion, activation and memory markers on final TIL product were maintained between Gen 3.1 Test and Gen 3.0 process.

IFN-γ production on final TIL product was 3 times higher on Gen 3.1 with feeder and OKT-3 addition on day 0, compared to Gen 3.0 in the two tumors analyzed, suggesting Gen 3.1 process generated a potent TIL product.

No differences observed in glucose or lactate levels across test conditions. No differences observed on glutamine and ammonia between Gen 3.0 and Gen 3.1 processes across media conditions. The low levels of glutamine on the media are not limiting cell growth and suggest the addition of GlutaMax™ only in media is sufficient to give the nutrients needed to make cells proliferate.

The scale up on day 11 and day 10 respectively and did not show major differences in terms of cell number reached on the harvest day of the process and metabolite consumption was comparable in both cases during the whole process. This observation suggests of Gen 3.0 optimized process can have flexibility on processing days, thereby facilitating flexibility in the manufacturing schedule.

Gen 3.1 process with feeder and OKT-3 addition on day 0 showed a higher clonal diversity measured by CDR3 TCRab sequence analysis compared to Gen 3.0.

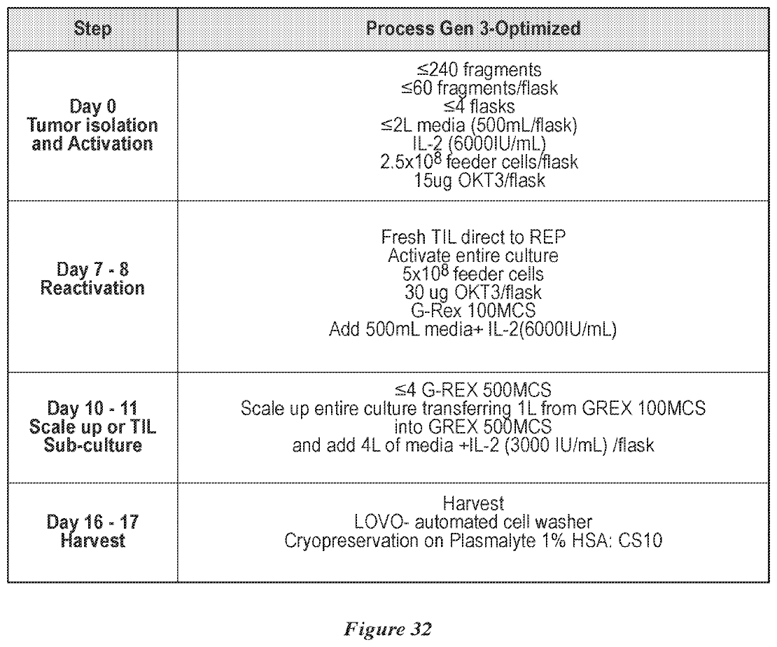

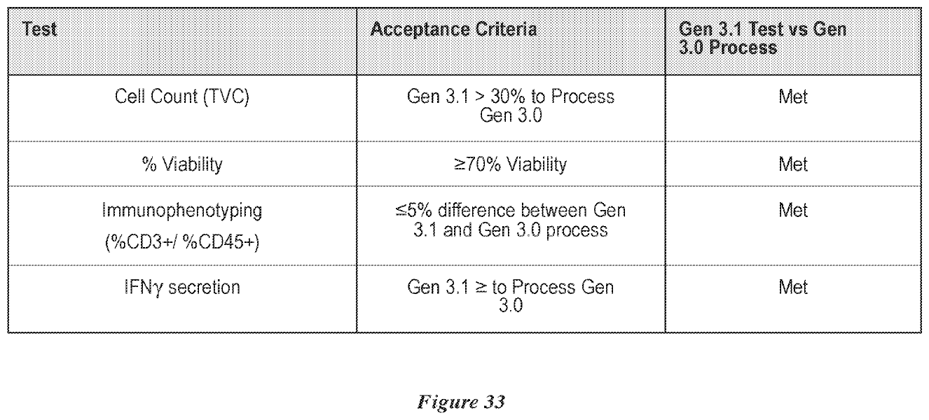

FIG. 32 describes an embodiment of the Gen 3 process (Gen 3 Optimized process). Standard media and CTS Optimizer serum free media can be used for Gen 3 Optimized process TIL expansion. In case of CTS Optimizer serum free media is recommended to increase the GlutaMax™ on the media to final concentration 4 mM.

### Example 11: Production and Characterization of Modified Tils with Membrane Anchored IL-15 and IL-21 Immunomodulatory Fusion Proteins

### Materials and Methods

Virus preparation and T cell transduction

To make lentivirus, pLenti-vectors containing tethered cytokine gene sequence (Table 58) and packaging helper vectors (VSV-G, Gag/Pol) were con-transfected into 293T cells. Lentivirus supernatant was collected from day 2-3 culture supernatants followed by ultracentrifuge (120,000 g) to concentrate lentivirus for TIL transduction. Before T cell transduction, pre-REP cells were stimulated with TransACT (1:100) for 2 days. E5 activated pre-REP cells were added into Retronectin pre-coated 48 well plates with concentrated lentivirus. Two days after gene transduction, pre-REP cells were harvested for REP process or further phenotypic characterization and functional assays.

TABLE 58
Membrane anchored IL-15 and IL-21 fusion protein DNA sequences

| Construct | DNA sequence |
|---|---|
| teIL-15 | atggactggacttggatactctttctggtcgctgccgccacacgggtgcactctaattgggtcaacgtgatca |
| | gcgacctgaagaagatcgaggacctgatccagagcatgcacatcgacgccacactgtacaccgagtccg |
| | atgtgcaccctagctgcaaagtgaccgccatgaagtgctttctgctggaactgcaagtgatcagcctggaa |
| | agcggcgacgccagcatccacgataccgtggaaaatctgatcatcctggccaacaacagcctgtccagc |
| | aacggcaatgtgaccgagagcggctgcaaagagtgcgaggaactggaagaaaagaacatcaaagagtt |
| | tctgcagagcttcgtccacatcgtgcagatgttcatcaacacctcatcaggtggcggtggaagcggaggtg |
| | gcggtagtggcggcggaggctcaggcggcggaggttccggcggaggatctcttcagctcctgccatctt |
| | gggctatcaccctgattagtgtgaatgggatctttgtcatctgttgtctcacgtactgtttcgctccccggtgca |
| | gagagagaaggcgcaacgaaagactgcggagagaaagcgtcagacccgtg (SEQ ID |
| | NO: 296) |
| teIL-21 | atggattggacctggattctgttcctggtggccgctgccacaagagtgcatagccagggccaagaccggc |
| | acatgatccggatgagacagctgatcgacatcgtggaccagctgaagaactacgtgaacgacctggtgcc |
| | tgagttcctgcctgctcctgaggacgtggaaacaaattgcgagtggtccgccttcagctgcttccagaagg |
| | cccagctgaaaagcgccaacaccggcaacaacgagcggatcatcaacgtgtccatcaagaagctgaag |
| | cggaagcctcctagcaccaatgccggaagaaggcagaagcacagactgacctgtcctagctgcgacag |
| | ctacgagaagaagcctccaaaagagttcctggaacggttcaagagcctgctgcagaagatgatccaccag |
| | cacctgagcagcagaacccacggctctgaagattctagcggaggcggaggaagtggtggcggaggttct |
| | ggtggcggtggatcaggcggtggcggatctggcggcggaggcagtggcggaggtggaagcggtggtg |
| | gtggctctggcggaggcggtagcggcggaggcggatctcttcaattgctgcctagctgggccatcacact |
| | gatctccgtgaacggcatcttcgtgatctgctgcctgacctactgcttcgcccctagatgcagagagcggag |
| | aagaaacgagcggctgagaagagaatctgtgcggcctgtt (SEQ ID NO: 297) |
| teIL-15/IL21 | atggattggacctggattctgttcctggtggccgctgccacaagagtgcatagccagggccaagaccggc |
| | acatgatccggatgagacagctgatcgacatcgtggaccagctgaagaactacgtgaacgacctggtgcc |
| | tgagttcctgcctgctcctgaggacgtggaaacaaattgcgagtggtccgccttcagctgcttccagaagg |
| | cccagctgaaaagcgccaacaccggcaacaacgagcggatcatcaacgtgtccatcaagaagctgaag |
| | cggaagcctcctagcaccaatgccggaagaaggcagaagcacagactgacctgtcctagctgcgacag |
| | ctacgagaagaagcctccaaaagagttcctggaacggttcaagagcctgctgcagaagatgatccaccag |
| | cacctgagcagcagaacccacggctctgaagattctagcggaggcggaggaagtggtggcggaggttct |
| | ggtggcggtggatcaggcggtggcggatctggcggcggaggcagtggcggaggtggaagcggtggtg |
| | gtggctctggcggaggcggtagcggcggaggcggatctcttcaattgctgcctagctgggccatcacact |
| | gatctccgtgaacggcatcttcgtgatctgctgcctgacctactgcttcgcccctagatgcagagagcggag |
| | aagaaacgagcggctgagaagagaatctgtgcggcctgttagagccaagagatctggaagcggcgcca |
| | ccaactttagcctgctgaaacaggctggcgacgtggaagagaaccctggacctatggactggacttggat |
| | actctttctggtcgctgccgccacacgggtgcactctaattgggtcaacgtgatcagcgacctgaagaagat |
| | cgaggacctgatccagagcatgcacatcgacgccacactgtacaccgagtccgatgtgcaccctagctgc |
| | aaagtgaccgccatgaagtgctttctgctggaactgcaagtgatcagcctggaaagcggcgacgccagca |
| | tccacgataccgtggaaaatctgatcatcctggccaacaacagcctgtccagcaacggcaatgtgaccga |
| | gagcggctgcaaagagtgcgaggaactggaagaaaagaacatcaaagagtttctgcagagcttcgtcca |
| | catcgtgcagatgttcatcaacacctcatcaggtggcggtggaagcggaggtggcggtagtggcggcgg |
| | aggctcaggcggcggaggttccggcggaggatctcttcagctcctgccatcttgggctatcaccctgatta |
| | gtgtgaatgggatctttgtcatctgttgtctcacgtactgtttcgctccccggtgcagagagagaaggcgcaa |
| | cgaaagactgcggagagaaagcgtcagacccgtg (SEQ ID NO: 298) |

### Flow Cytometry

To check mIL-15 expression in transduced cells, biotin-conjugate IL-15 (biolgend, San Diego, CA) plus streptavidin-BV421 (Biolegend, San Diego, CA) were used to stain transduced cells. To check mIL-21 expression, PE-conjugated IL-21 antibodies were used to stain transduced cells. To check T cell division, CellTrace™ Violet Cell Proliferation dye (ThermoFisher scientific, Waltham, MA) was used to pre-label T cells. T cell division was analyzed after 5 days in ex vivo culture in presence or absence of IL-2 (201U/ml). To check T cell activation and differentiation, antibodies from BD Bioscience, Biolegend, Thermofisher were used for phenotypic characterization. Data was acquired with a BioRad ZE5 flow cytometer and analyzed with FlowJo software (FlowJo, LLC, Ashland, OR).

### T Cell Count and Viability

REP cells were harvested after TIL expansion. Live cell numbers were assessed by AOPI assay using Cellometer K2 cell counter machine (Nexcelom, Lawrence, MA).

### Pre-REP and REP Process

Human tumor samples were dissected into −3 mm pieces and cultured in Grex10 with recommended culture medium containing IL-2 (6000 IU/ml). After 11 days of culture, pre-REP cells were harvested for 2-day activation and 2-day lentiviral transduction. Transduced Pre-REP cells were then propagated in a REP expansion process with irradiated PBMCs, anti-CD3 antibody and 3,000 IU/ml of IL-2 for another 11 days.

### Results

Expression of mIL-15/mIL-21 after Gene Transduction

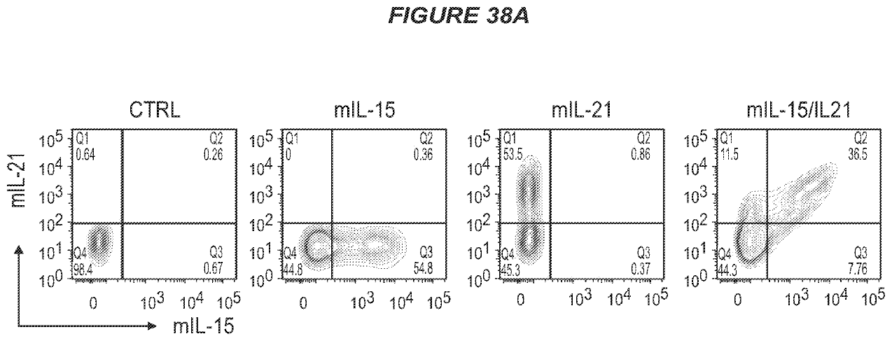

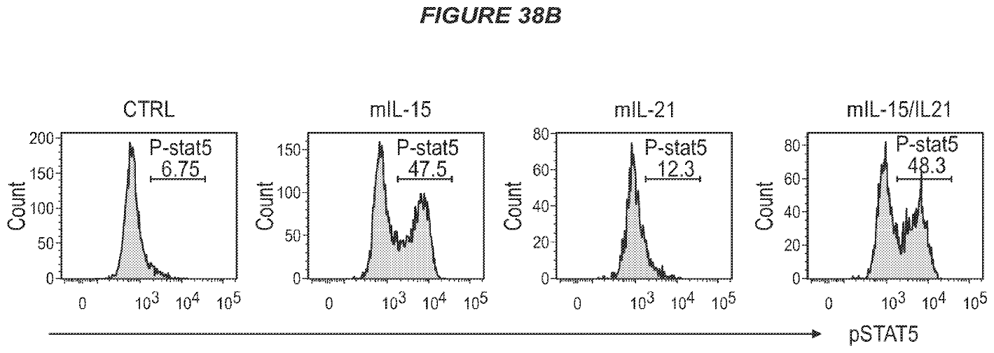

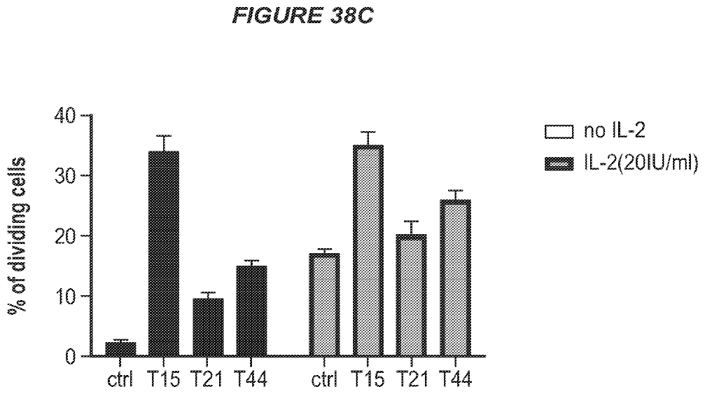

After gene transduction, transduced pre-REP cells were ex vivo expanded with 3001U/ml IL-2 for another 5 days. By flow cytometry staining, transduced pre-REP cells exhibited expression of mIL-15 and mIL-21. As shown in FIG. 38A, there was a 54.8% expression of mIL-15 in mIL-15 lentiviral transduced TILs, a 53.5% expression of mIL-21 in mIL-21 transduced TILs and a 36.5% expression of mIL-15/mIL-21 double positive cells in mIL-15/IL21 transduced TILs. Un-transduced cells were used as negative control.

mIL-15 Pre-REP Cells Exhibit Sustained Activation of pSTAT5 Signaling and Showed Increased Proliferation

After verification of surface cytokine expression, T cell functionality was examined. Phosphorylation of STATS is a hallmark of T cell activation upon gamma chain cytokine stimulation. In conditions of serum starvation, mIL-15, mIL-21, and mIL-15/IL-21 lentiviral transduced cells displayed sustained pSTAT5 signaling indicating that mIL-15 is functional. As shown in FIG. 38B, mIL-15 is superior to mIL-21 in inducing pSTAT5 activation. Further, cell proliferation of mIL-15 lentiviral transduced TILs was examined by CellTrace proliferation assay. CellTrace violet pre-labeled TILs were ex vivo cultured for 5 days in the presence or absence of IL-2. As shown in FIG. 38C, mIL-15 TILs exhibit superior proliferative capacity in comparison with mIL-21 or mIL-15/IL-21 TILs in the presence or absence of IL-2. mIL-15 TILs can proliferate in the absence of IL-2, indicating mIL-15 can substitute for IL-2 to provide a proliferative signal.

Expression of mIL-15/mIL-21 after REP Process

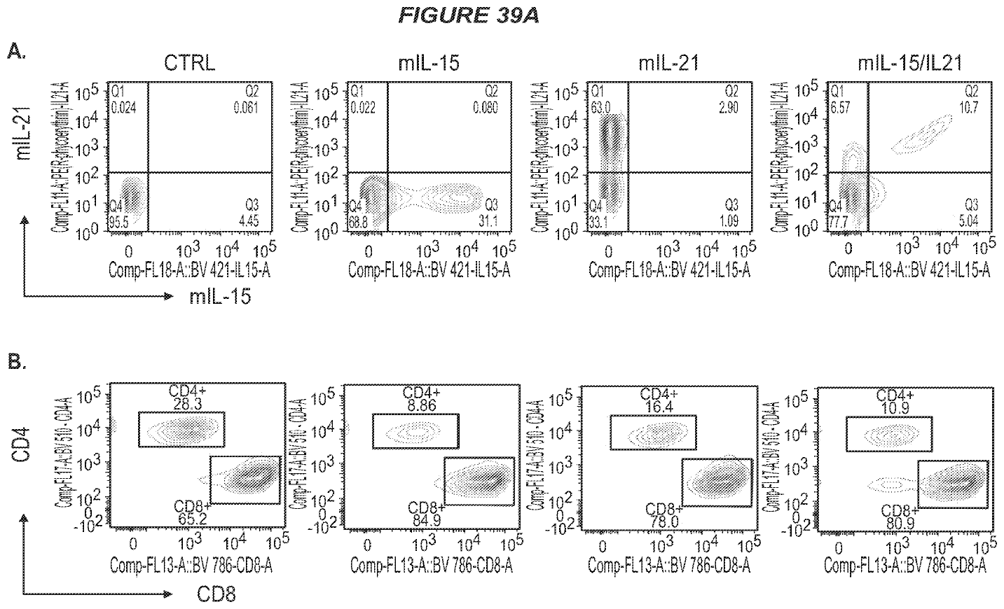

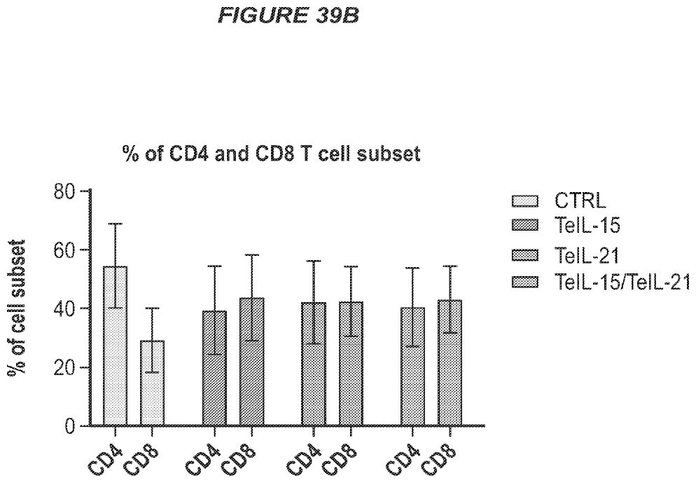

After lentiviral transduction, TILs were expanded for 11 days in a REP process. Surface expression of membrane-bound cytokine receptors were then assessed by flow cytometry. As shown in FIG. 39A, there was a 31.1% expression of mIL-15 in mIL-15 TILs, a 63% expression of mIL-21 in mIL-21 TILs and a 10.7% expression of mIL-15/mIL-21 double positive cells in mIL-15/IL21 TILs. Un-transduced cells were used as a negative control.

Expression of mIL-15/mIL-21 Promote CD8 T Cell Expansion in REP Process

In harvested REP cells, an increased percentage of CD8+ T cells was detected in mIL-15, mIL-21 and mIL-15/IL-21 lentiviral transduced TILs (FIGS. 39B and 39C). This is consistent with previous reports showing that IL-15 and IL-21 act as a potent stimulator, which preferentially stimulates the proliferation and survival of CD8 T cells.

Phenotype of mIL-15/mIL-21 Expressing REP TIL

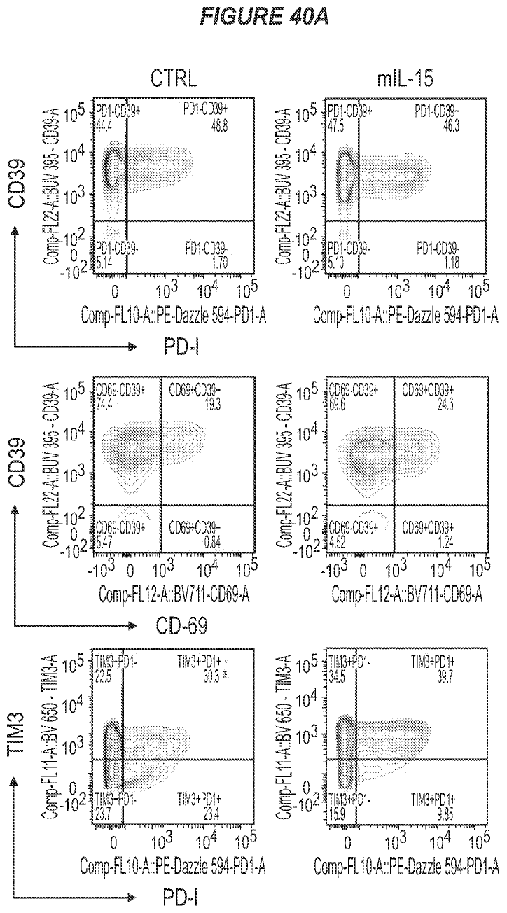

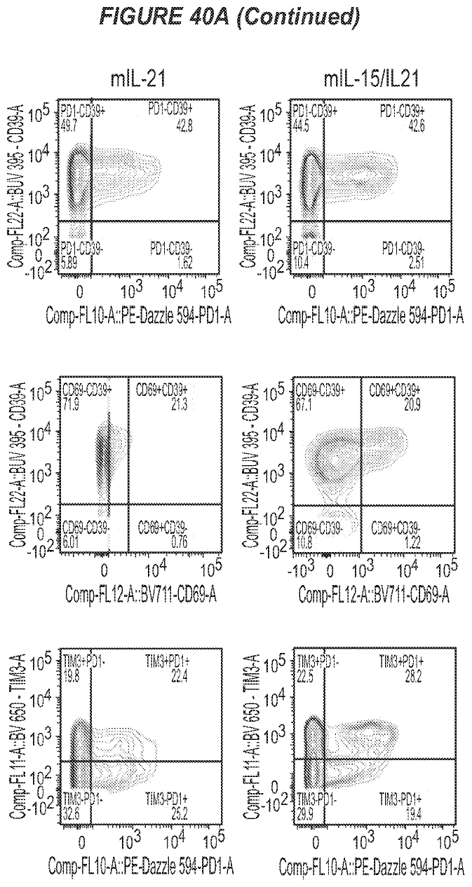

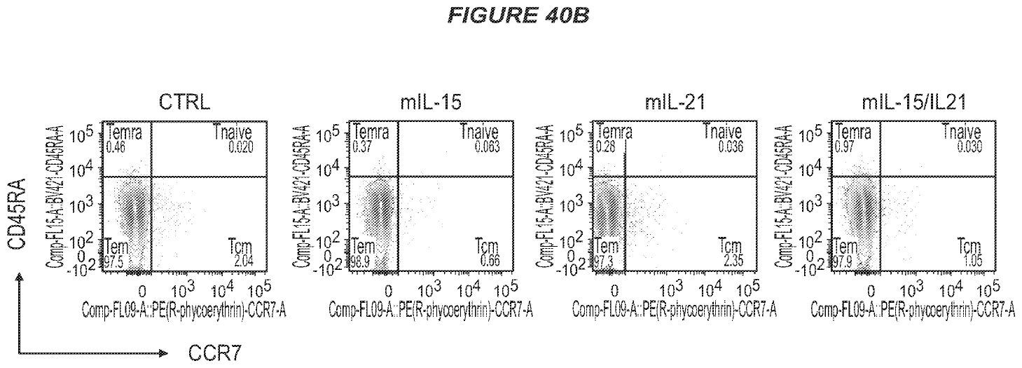

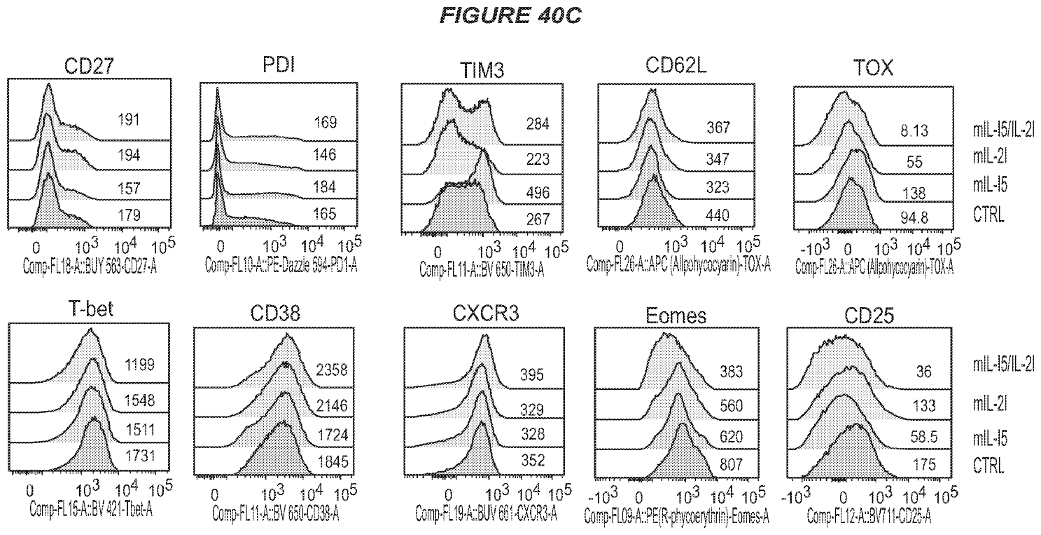

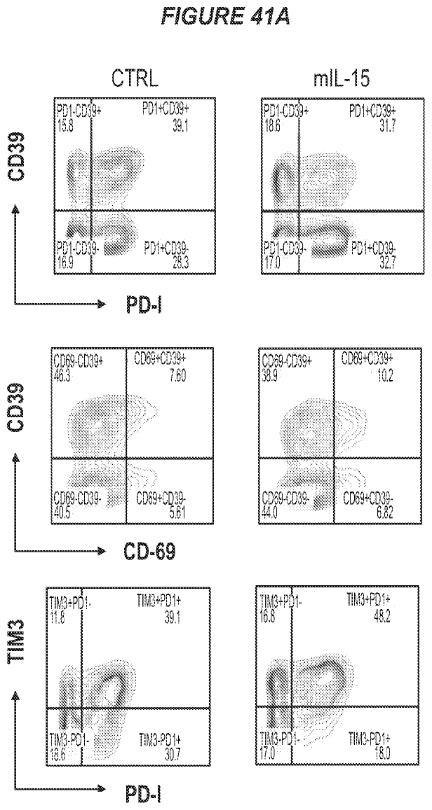

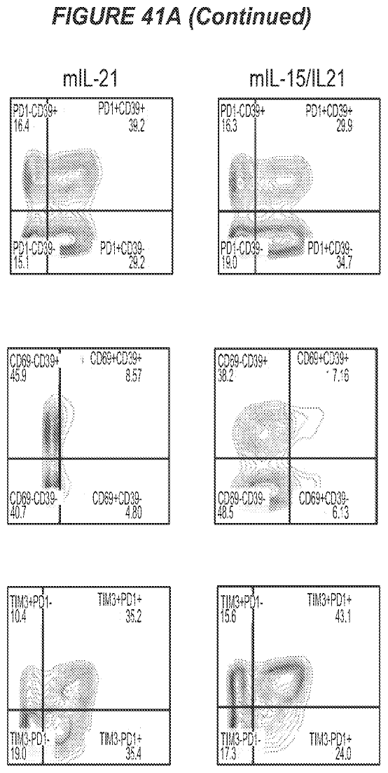

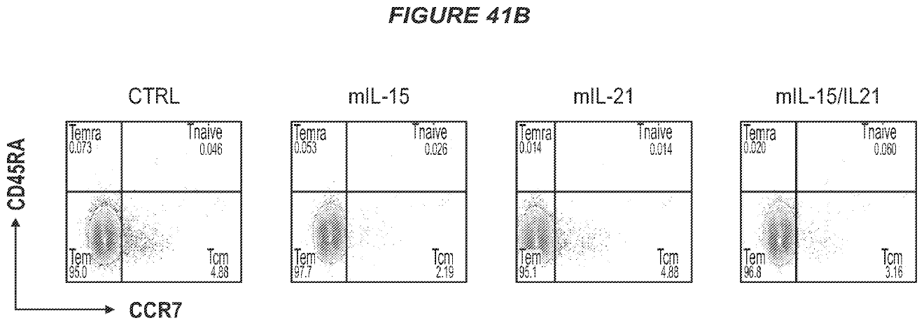

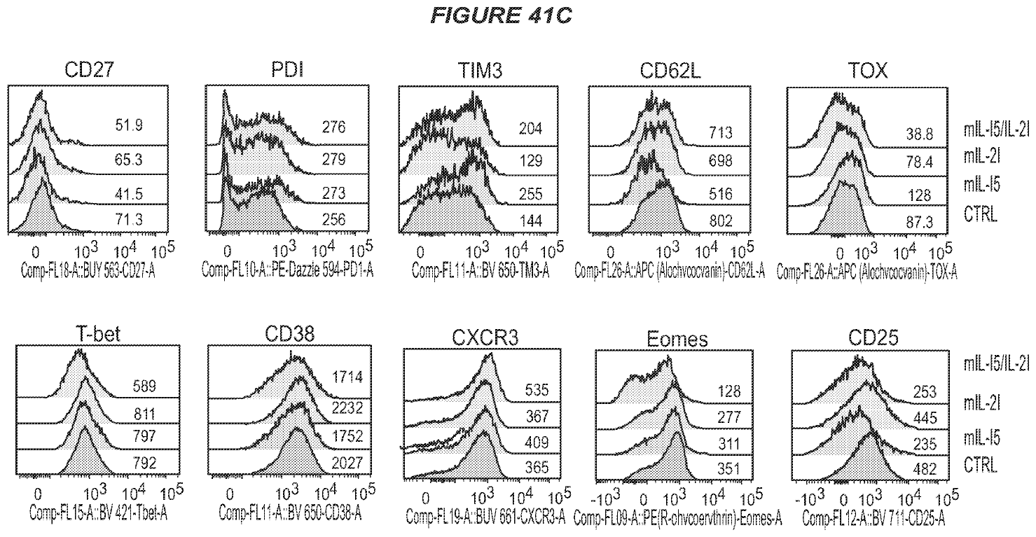

To characterize the immune regulatory effect of mIL-15 and mIL-21 on REP cells, phenotypic analysis was performed with freshly thawed REP cells in both gated CD8+CD3+ T cells (FIG. 40) and CD4+CD3+ T cell subset (FIG. 41). We found mbIL-15 significantly downregulate CD25 (IL-2Ralpha) expression. As IL-2, IL-15 are in competition in using IL-2R beta and IL-2R gamma, suggesting a negative feedback mechanism in regulating CD25. Also, mIL-15 appears to activate T cells, characterized with higher expression of TIM3, TOX, while IL-21 exhibit antagonist effect. Further, both mIL-15 and mIL-21 downregulate expression of Eomes. No large difference was observed in the expression of other surface marker such as PD-1, CD27, CXCR3, etc.

### Example 12: Production and Characterization of Modified Tils with Membrane Anchored IL-15 and IL-21 Immunomodulatory Fusion Proteins

For this study, modified TILs with membrane anchored IL-15 and/or IL-21 immunodulatory fusion proteins will be made, wherein expression of the membrane anchored immunodoluatory IL-15 and IL-21 is controlled using a NFAT promoter that includes NFAT responsive elements linked to a minimal human IL-2 promoter, see, e.g., Table 59. The modified TILs will be characterized as set forth below.

### Materials and Methods

### Virus Preparation and T Cell Transduction

To make lentivirus, pLenti-vectors containing the tethered cytokine gene sequence (Table 59) and packaging helper vectors (BaEV-TR, Gag/Pol) will be co-transfected into 293T cells. Lentivirus supernatant will be collected from day 2-3 culture supernatants followed by ultracentrifuge (120,000 g) to concentrate lentivirus for TIL transduction. 1E5 pre-REP cells will be added into Retronectin pre-coated 48 well plates with concentrated lentivirus. Two days after gene transduction, pre-REP cells will be harvested for REP process or further phenotypic characterization and functional assays.

TABLE 59
Membrane anchored IL-12, IL-15 and IL-21 DNA sequences (with NFAT
Promoter)

| Construct | DNA sequence |
|---|---|
| NFAT- | tcgaggtcgacggtatcgataagcttgatatcgaattaggaggaaaaactgtttcatacagaaggcgtcaattag |
| Te-IL12 | gaggaaaaactgtttcatacagaaggcgtcaattaggaggaaaaactgtttcatacagaaggcgtcaattggtcc |
| DNA seq | catcgaattaggaggaaaaactgtttcatacagaaggcgtcaattaggaggaaaaactgtttcatacagaaggc |
| | gtcaattaggaggaaaaactgtttcatacagaaggcgtcaattggtcccgggacattttgacacccccataatattt |
| | ttccagaattaacagtataaattgcatctcttgttcaagagttccctatcactctctttaatcactactcacagtaacct |
| | caactcctggccaccATGGATTGGACCTGGATTCTGTTCCTCGTGGCCGCCG |
| | CTACCCGCGTGCACTCCATCTGGGAGCTGAAGAAAGACGTGTACGT |
| | GGTGGAATTGGATTGGTACCCCGACGCCCCAGGCGAGATGGTGGTT |
| | CTGACTTGCGACACGCCGGAGGAGGACGGCATCACCTGGACGCTG |
| | GACCAGAGCAGCGAAGTGCTGGGCTCTGGTAAGACCTTAACCATCC |
| | AGGTCAAGGAGTTCGGTGATGCAGGACAGTACACGTGTCACAAGG |
| | GCGGGGAGGTCTTGTCCCATAGCCTGCTGCTCCTGCACAAGAAGGA |
| | AGACGGCATCTGGTCTACGGATATCCTTAAAGACCAGAAGGAGCC |
| | CAAGAACAAAACCTTCCTGCGATGCGAGGCTAAGAATTACTCCGG |
| | GCGCTTTACCTGTTGGTGGCTCACAACCATCTCGACCGACCTGACC |
| | TTCTCCGTGAAGTCTTCCAGGGGATCTTCTGATCCGCAAGGCGTTA |
| | CCTGCGGGGCCGCTACCCTGTCGGCTGAGCGCGTCCGTGGTGACAA |
| | CAAGGAGTACGAGTACTCGGTGGAATGCCAGGAGGACAGCGCCTG |
| | CCCAGCGGCCGAAGAGTCCTTGCCCATCGAGGTGATGGTGGACGCT |
| | GTACACAAGCTCAAGTACGAGAACTACACCTCCTCCTTCTTTATAC |
| | GCGACATCATCAAGCCTGACCCCCCTAAAAACCTGCAGCTGAAGCC |
| | CCTTAAAAACTCTCGACAGGTCGAGGTATCCTGGGAGTACCCCGAC |
| | ACCTGGTCTACTCCTCATTCGTATTTCAGCCTGACCTTCTGCGTGCA |
| | GGTGCAGGGCAAGTCCAAGCGCGAGAAGAAGGACCGCGTGTTCAC |
| | CGACAAAACCAGCGCGACTGTCATTTGTCGGAAGAACGCGAGCAT |
| | CTCCGTACGGGCACAGGATCGCTATTACTCCAGCAGTTGGTCAGAG |
| | TGGGCTTCGGTTCCGTGTTCTGGAGGAGGTGGCGGCGGCTCACGCA |
| | ACCTGCCCGTGGCGACGCCGGACCCGGGTATGTTTCCTTGTCTGCA |
| | CCACTCTCAGAACTTGCTCCGTGCCGTCTCGAATATGCTGCAGAAG |
| | GCCCGCCAGACTCTGGAGTTCTATCCATGCACTAGTGAGGAGATCG |
| | ATCATGAGGACATCACCAAGGACAAGACATCCACCGTGGAGGCTT |
| | GTCTGCCGCTGGAGTTGACAAAGAACGAGAGCTGCCTCAATAGCC |
| | GCGAGACTTCCTTCATCACCAACGGCTCCTGCCTCGCGAGCCGTAA |
| | GACCTCCTTTATGATGGCGCTGTGCCTGTCAAGTATCTACGAAGAT |
| | CTGAAAATGTATCAGGTGGAGTTCAAAACTATGAACGCAAAACTCC |
| | TGATGGATCCCAAGCGCCAAATTTTCTTGGACCAGAACATGCTTGC |
| | CGTGATCGACGAGCTGATGCAGGCCCTGAACTTCAACTCCGAGACC |
| | GTCCCTCAGAAGTCGTCTCTGGAGGAACCCGACTTTTACAAGACAA |
| | AGATCAAGCTGTGCATCCTGCTACACGCGTTCCGCATTCGGGCCGT |
| | GACCATTGACCGCGTGATGTCCTACCTGAATGCCTCGTCCGGCGGG |
| | GGGGGCTCCGGTGGGGGCGGCTCTGGCGGAGGCGGCTCAGGTGGC |
| | GGCGGCTCCGGCGGAGGTTCGCTTCAGCTTCTGCCCAGTTGGGCAA |
| | TCACTCTGATTTCCGTCAACGGAATCTTCGTCATCTGCTGCCTGACC |
| | TACTGCTTTGCTCCCAGATGTAGAGAGCGCCGCCGCAACGAGCGCC |
| | TGCGTCGTGAGAGCGTGAGGCCTGTGTGA |
| | (SEQ ID NO: 299) |
| NFAT- | tcgaggtcgacggtatcgataagcttgatatcgaattaggaggaaaaactgtttcatacagaaggcgtcaattag |
| Te-IL15 | gaggaaaaactgtttcatacagaaggcgtcaattaggaggaaaaactgtttcatacagaaggcgtcaattggtcc |
| DNA seq | catcgaattaggaggaaaaactgtttcatacagaaggcgtcaattaggaggaaaaactgtttcatacagaaggc |
| | gtcaattaggaggaaaaactgtttcatacagaaggcgtcaattggtcccgggacattttgacacccccataatattt |
| | ttccagaattaacagtataaattgcatctcttgttcaagagttccctatcactctctttaatcactactcacagtaacct |
| | caactcctggccaccATGGACTGGACTTGGATACTCTTTCTGGTCGCTGCCG |
| | CCACACGGGTGCACTCTAATTGGGTCAACGTGATCAGCGACCTGAA |
| | GAAGATCGAGGACCTGATCCAGAGCATGCACATCGACGCCACACT |
| | GTACACCGAGTCCGATGTGCACCCTAGCTGCAAAGTGACCGCCATG |
| | AAGTGCTTTCTGCTGGAACTGCAAGTGATCAGCCTGGAAAGCGGCG |
| | ACGCCAGCATCCACGATACCGTGGAAAATCTGATCATCCTGGCCAA |
| | CAACAGCCTGTCCAGCAACGGCAATGTGACCGAGAGCGGCTGCAA |
| | AGAGTGCGAGGAACTGGAAGAAAAGAACATCAAAGAGTTTCTGCA |
| | GAGCTTCGTCCACATCGTGCAGATGTTCATCAACACCTCATCAGGT |
| | GGCGGTGGAAGCGGAGGTGGCGGTAGTGGCGGCGGAGGCTCAGGC |
| | GGCGGAGGTTCCGGCGGAGGATCTCTTCAGCTCCTGCCATCTTGGG |
| | CTATCACCCTGATTAGTGTGAATGGGATCTTTGTCATCTGTTGTCTC |
| | ACGTACTGTTTCGCTCCCCGGTGCAGAGAGAGAAGGCGCAACGAA |
| | AGACTGCGGAGAGAAAGCGTCAGACCCGTGTGA (SEQ ID NO: 300) |
| NFAT- | tcgaggtcgacggtatcgataagcttgatatcgaattaggaggaaaaactgtttcatacagaaggcgtcaattag |
| TeIL21 | gaggaaaaactgtttcatacagaaggcgtcaattaggaggaaaaactgtttcatacagaaggcgtcaattggtcc |
| DNA seq | catcgaattaggaggaaaaactgtttcatacagaaggcgtcaattaggaggaaaaactgtttcatacagaaggc |
| | gtcaattaggaggaaaaactgtttcatacagaaggcgtcaattggtcccgggacattttgacacccccataatattt |
| | ttccagaattaacagtataaattgcatctcttgttcaagagttccctatcactctctttaatcactactcacagtaacct |
| | caactcctggccaccATGGATTGGACCTGGATTCTGTTCCTGGTGGCCGCTG |
| | CCACAAGAGTGCATAGCCAGGGCCAAGACCGGCACATGATCCGGA |
| | TGAGACAGCTGATCGACATCGTGGACCAGCTGAAGAACTACGTGA |
| | ACGACCTGGTGCCTGAGTTCCTGCCTGCTCCTGAGGACGTGGAAAC |
| | AAATTGCGAGTGGTCCGCCTTCAGCTGCTTCCAGAAGGCCCAGCTG |
| | AAAAGCGCCAACACCGGCAACAACGAGCGGATCATCAACGTGTCC |
| | ATCAAGAAGCTGAAGCGGAAGCCTCCTAGCACCAATGCCGGAAGA |
| | AGGCAGAAGCACAGACTGACCTGTCCTAGCTGCGACAGCTACGAG |
| | AAGAAGCCTCCAAAAGAGTTCCTGGAACGGTTCAAGAGCCTGCTG |
| | CAGAAGATGATCCACCAGCACCTGAGCAGCAGAACCCACGGCTCT |
| | GAAGATTCTAGCGGAGGCGGAGGAAGTGGTGGCGGAGGTTCTGGT |
| | GGCGGTGGATCAGGCGGTGGCGGATCTGGCGGCGGAGGCAGTGGC |
| | GGAGGTGGAAGCGGTGGTGGTGGCTCTGGCGGAGGCGGTAGCGGC |
| | GGAGGCGGATCTCTTCAATTGCTGCCTAGCTGGGCCATCACACTGA |
| | TCTCCGTGAACGGCATCTTCGTGATCTGCTGCCTGACCTACTGCTTC |
| | GCCCCTAGATGCAGAGAGCGGAGAAGAAACGAGCGGCTGAGAAG |
| | AGAATCTGTGCGGCCTGTTTAA (SEQ ID NO: 301) |
| NFAT- | tcgaggtcgacggtatcgataagcttgatatcgaattaggaggaaaaactgtttcatacagaaggcgtcaattag |
| TeIL 15- | gaggaaaaactgtttcatacagaaggcgtcaattaggaggaaaaactgtttcatacagaaggcgtcaattggtcc |
| IL21 | catcgaattaggaggaaaaactgtttcatacagaaggcgtcaattaggaggaaaaactgtttcatacagaaggc |
| DNA seq | gtcaattaggaggaaaaactgtttcatacagaaggcgtcaattggtcccgggacattttgacacccccataatattt |
| | ttccagaattaacagtataaattgcatctcttgttcaagagttccctatcactctctttaatcactactcacagtaacct |
| | caactcctggccaccATGGATTGGACCTGGATTCTGTTCCTGGTGGCCGCTG |
| | CCACAAGAGTGCATAGCCAGGGCCAAGACCGGCACATGATCCGGA |
| | TGAGACAGCTGATCGACATCGTGGACCAGCTGAAGAACTACGTGA |
| | ACGACCTGGTGCCTGAGTTCCTGCCTGCTCCTGAGGACGTGGAAAC |
| | AAATTGCGAGTGGTCCGCCTTCAGCTGCTTCCAGAAGGCCCAGCTG |
| | AAAAGCGCCAACACCGGCAACAACGAGCGGATCATCAACGTGTCC |
| | ATCAAGAAGCTGAAGCGGAAGCCTCCTAGCACCAATGCCGGAAGA |
| | AGGCAGAAGCACAGACTGACCTGTCCTAGCTGCGACAGCTACGAG |
| | AAGAAGCCTCCAAAAGAGTTCCTGGAACGGTTCAAGAGCCTGCTG |
| | CAGAAGATGATCCACCAGCACCTGAGCAGCAGAACCCACGGCTCT |
| | GAAGATTCTAGCGGAGGCGGAGGAAGTGGTGGCGGAGGTTCTGGT |
| | GGCGGTGGATCAGGCGGTGGCGGATCTGGCGGCGGAGGCAGTGGC |
| | GGAGGTGGAAGCGGTGGTGGTGGCTCTGGCGGAGGCGGTAGCGGC |
| | GGAGGCGGATCTCTTCAATTGCTGCCTAGCTGGGCCATCACACTGA |
| | TCTCCGTGAACGGCATCTTCGTGATCTGCTGCCTGACCTACTGCTTC |
| | GCCCCTAGATGCAGAGAGCGGAGAAGAAACGAGCGGCTGAGAAG |
| | AGAATCTGTGCGGCCTGTTAGAGCCAAGAGATCTGGAAGCGGCGC |
| | CACCAACTTTAGCCTGCTGAAACAGGCTGGCGACGTGGAAGAGAA |
| | CCCTGGACCTATGGACTGGACTTGGATACTCTTTCTGGTCGCTGCCG |
| | CCACACGGGTGCACTCTAATTGGGTCAACGTGATCAGCGACCTGAA |
| | GAAGATCGAGGACCTGATCCAGAGCATGCACATCGACGCCACACT |
| | GTACACCGAGTCCGATGTGCACCCTAGCTGCAAAGTGACCGCCATG |
| | AAGTGCTTTCTGCTGGAACTGCAAGTGATCAGCCTGGAAAGCGGCG |
| | ACGCCAGCATCCACGATACCGTGGAAAATCTGATCATCCTGGCCAA |
| | CAACAGCCTGTCCAGCAACGGCAATGTGACCGAGAGCGGCTGCAA |
| | AGAGTGCGAGGAACTGGAAGAAAAGAACATCAAAGAGTTTCTGCA |
| | GAGCTTCGTCCACATCGTGCAGATGTTCATCAACACCTCATCAGGT |
| | GGCGGTGGAAGCGGAGGTGGCGGTAGTGGCGGCGGAGGCTCAGGC |
| | GGCGGAGGTTCCGGCGGAGGATCTCTTCAGCTCCTGCCATCTTGGG |
| | CTATCACCCTGATTAGTGTGAATGGGATCTTTGTCATCTGTTGTCTC |
| | ACGTACTGTTTCGCTCCCCGGTGCAGAGAGAGAAGGCGCAACGAA |
| | AGACTGCGGAGAGAAAGCGTCAGACCCGTGTAA (SEQ ID NO: 302) |

### Flow Cytometry

To check mInL-15 expression in transduced cells, biotin-conjugate IL-15 (biolgend, San Diego, CA) plus streptavidin-BV421 (Biolegend, San Diego, CA) will be used to stain transduced cells. To check mIL-21 expression, PE-conjugated IL-21 antibodies will be used to stain transduced cells. To check T cell division, CellTrace™ Violet Cell Proliferation dye (ThermoFisher scientific, Waltham, MA) will be used to pre-label T cells. T cell division was analyzed after 5 days in ex vivo culture in presence or absence of IL-2 (201U/ml). To check T cell activation and differentiation, antibodies from BD Bioscience, Biolegend, Thermofisher will be used for phenotypic characterization. Data was acquired with a BioRad ZE5 flow cytometer and analyzed with FlowJo software (FlowJo, LLC, Ashland, OR).

### T Cell Count and Viability

REP cells will be harvested after TIL expansion. Live cell numbers will be assessed by AOPI assay using Cellometer K2 cell counter machine (Nexcelom, Lawrence, MA).

### Pre-REP and REP Process

Human tumor samples will be dissected into −3 mm pieces and cultured in Grex10 with recommended culture medium containing IL-2 (6000 IU/ml). After 11 days of culture, pre-REP cells will be harvested for 2-day activation and 2-day lentiviral transduction. Transduced Pre-REP cells will be then propagated in a REP expansion process with irradiated PBMCs, anti-CD3 antibody and 3,000 IU/ml of IL-2 for another 11 days.

### Results

Expression of mIL-15/mIL-21 after Gene Transduction

After gene transduction, transduced pre-REP cells will be ex vivo expanded with 3001U/ml IL-2 for another 5 days. By flow cytometry staining, transduced pre-REP cells will be assessed for expression of mIL-15 and mIL-21. Un-transduced cells will be used as negative control.

mIL-15 Pre-REP Cells Exhibit Sustained Activation of pSTAT5 Signaling and Showed Increased Proliferation

After verification of surface cytokine expression, T cell functionality will be examined. In particular, phosphorylation of STATS of the modified TILs will be assessed under serum starved conditions to determine if the modified TILs express function mIL-15 and/or IL-21. Further, cell proliferation of mIL-15 lentiviral transduced TILs will be examined by CellTrace proliferation assay.

Expression of mIL-15/mIL-21 after REP process

After lentiviral transduction, TILs will be expanded for 11 days in a REP process and surface expression of membrane-bound cytokine receptors will then be assessed by flow cytometry. Harvested REP cells will be assessed for CD8+ T cells. Previous reports show that IL-15 and IL-21 act as a potent stimulator, which preferentially stimulates the proliferation and survival of CD8 T cells.

Phenotype of mIL-15/mIL-21 Expressing REP TIL

To characterize the immune regulatory effect of mIL-15 and mIL-21 on REP cells, phenotypic analysis will be performed with freshly thawed REP cells in both gated CD8+CD3+ T cells and CD4+CD3+ T cell subset. Expression of the following will be assessed: CD27, PD1, TIM3, CD62L, TOX, T-bet, CD38, CXCR3, Eomes, and CD25.

### Example 13: Production and Characterization of Modified Tils with Membrane Anchored IL-2 and IL-12 Immunomodulatory Fusion Proteins

Four cryopreserved TIL populations will be thawed and cell numbers and viability will be assessed using a Cellaca MX counter. TILs will then be resuspended and mRNA encoding membrane anchored IL-2 and/or IL-12 (mbIL-2 and mb-IL12) will be delivered to subpopulations of the TILs using the SQZ method (i.e., “Squeezing”). Exemplary mbIL-2 and mbIL-12 constructs are depicted in FIG. 37. The following TIL subpulations will be assessed: 1) no SQZ; 2) no mRNA; 3) mbIL-2 mRNA; 4) mbIL-12 mRNA; and 5) mbIL-2+ mbIL-12 mRNA. Following Squeezing, cells will be assessed again for viability and numbers using a Cellaca MX counter. Cells will then be resuspended and cultured for 4 days in CM2 media with or without 3001U/ml of IL-2. Cells will be collected daily to determine cell numbers, viability and surface marker expression including, but not limited to: mbIL-2 and mIL-12. Following Squeezing, cells will also be stimulated with Transact in the presence or absence of 3001U/ml of IL-2. Cell numbers, viability and marker expression will also be evaluated together with IFNg and TNFa levels. Some TILs will also be cryopreserved following Squeezing and thawed after 1 week to follow expression of mbIL-2 and mbIL-12 after 1, 2, 3, and 4 days in culture with or without IL-2. This will allow for the assessment of whether Squeezed and cryopreserved cells maintain the same level of membrane-bound cytokine expression compared to fresh cells.

The functional effect of cells Squeezed with mbIL-2 and mbIL-12 will be evaluated in in vitro cytotoxicity assays using KILR THP-1 cells. Squeezed TILs will be cocultured at a 10:1 ratio with KILR THP-1 cells in the presence or absence of 3001U/ml of IL-2. 24 hrs later, cytotoxicity will be evaluated by addition of substrate to the culture plates. In a separate set of experiments, Squeezed and control TILs will be cocultured with KILR THP-1 cells for 3 days in the presence or absence of 3001U/ml of IL-2. Following this incubation period TIL numbers, viability and phenotype will be assessed.

Following this initial set of in vitro experiments, a PDX model will be run with control and mbIL-2 and mbIL-12 Squeezed TILs in both NSG and hIL-2 NOG mice. For these experiments, cryopreserved melanoma TILs with paired PDX tumor cells will be used. In brief, PDX tumor cells will be inoculated in NSG and hIL-2 NOG mice. Following tumor establishment, tumor bearing mice will be inoculated with control and Squeezed TILs (corresponding to the PDX model) at 10E6 cells/mouse via tail vein injection. Tumors and mouse weights will be measured biweekly.

After initial proof-of-concept (POC) experiments are completed as described above, two engineering runs will be performed using the Gen 2 process described in Example 7 above, and then the cell Squeeze process (as described above) will be performed to transfect the resulting TIL populations with mbIL-2 mRNA and/or mbIL-12 mRNA. TILs will be assessed for cell counts and viability pre and post Squeeze and marker expression will be assessed post Squeeze and evaluated for 4 days in the presence or absence of IL-2.

### Example 14: Production and Characterization of Modified Tils with Membrane Anchored IL-18 Immunomodulatory Fusion Proteins

IL-18 is a proinflammatory cytokine that facilitates type 1 immune response by enhancing IFN-g production (originally called IFNγ inducing factor). The immunostimulatory effects of IL-18 have been investigated for cancer treatments. Administration of recombinant human IL-18 (rhIL-18) showed very good safety profile, cancer patients can tolerate doses up to 2,000 μg/kg with generally mild-to-moderate toxicity (Clin Cancer Res 2008).

IL-18 binding protein (IL-18BP) is a secreted protein which can neutralize IL-18 and restrain biological function of IL-18. IL-18BP was found frequently upregulated in the tumor microenvironment of diverse human tumors. A decoy-resistant IL-18 (DR-IL18) was recently developed that exhibits resistance to IL-18BP inhibition, while maintaining signaling potential through binding to the IL-18R complex (Nature, 2020). Further, human IL-18 expressing CAR-T cells have been shown to exhibit robust enhanced proliferation and antitumor activity in a xenograft model. A clinical study with huCART19-IL18 is ongoing for the treatment of NHL/CLL Patients (Cell Report, 2017; NCT04684563).

Therefore, without being bound by any particular theory of operation, it is believed that TILs that include membrane anchored IL-18 immunomodulatory fusion proteins (i.e., tethered IL-18 TILs) as described and produced by the methods provided herein, can be useful for the treatment of cancers.

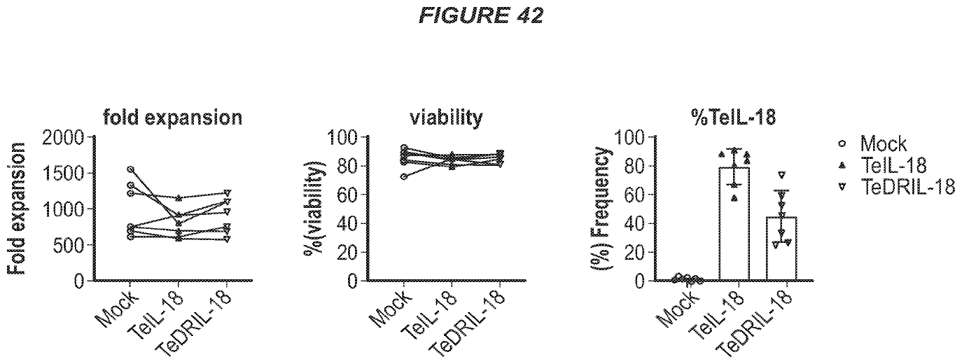

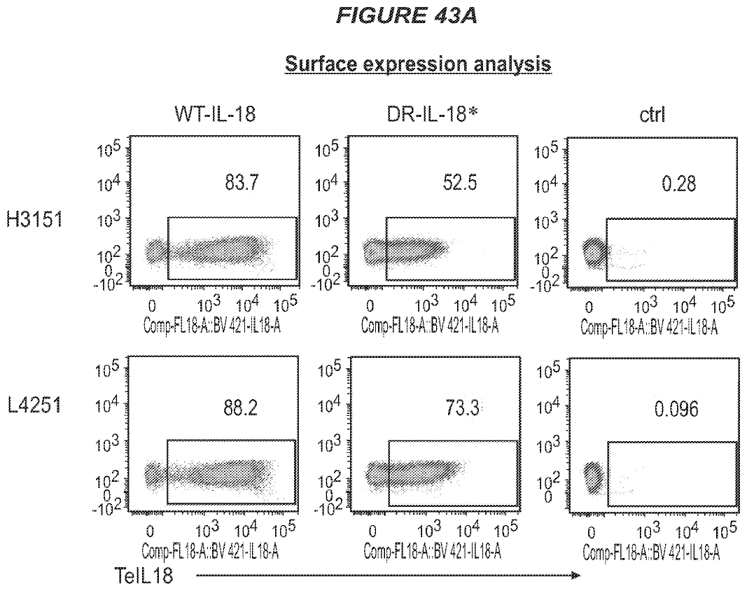

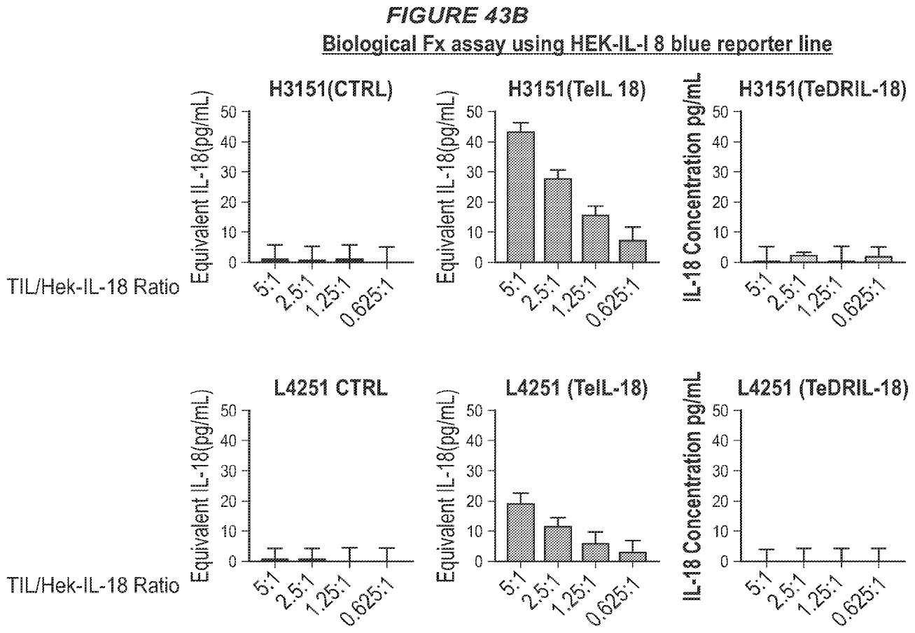

To characterize the properties of such tethered IL-18 TILs, TILs expressing member anchored IL18s (TeIL-18s) and DR-IL18 (TeDRIL-18s) were made by lentiviral gene transduction of constructs expressing these fusion proteins in combination with the 11-day REP process described herein. For this study, TILs derived from 7 different tumors were used: head and neck (3), lung (2), breast (1), and melanoma (1). These modified TILs exhibited no significant change with respect to TIL expansion and cell viability, as compared to control. See FIG. 42. As shown in FIGS. 43A and B, TeIL-18 and TeDRIL-18 expressing TILs produced through this process exhibited high expression levels of TeIL-18 and TeDRIL-18. Moreover, the high expression levels were sustained after REP.

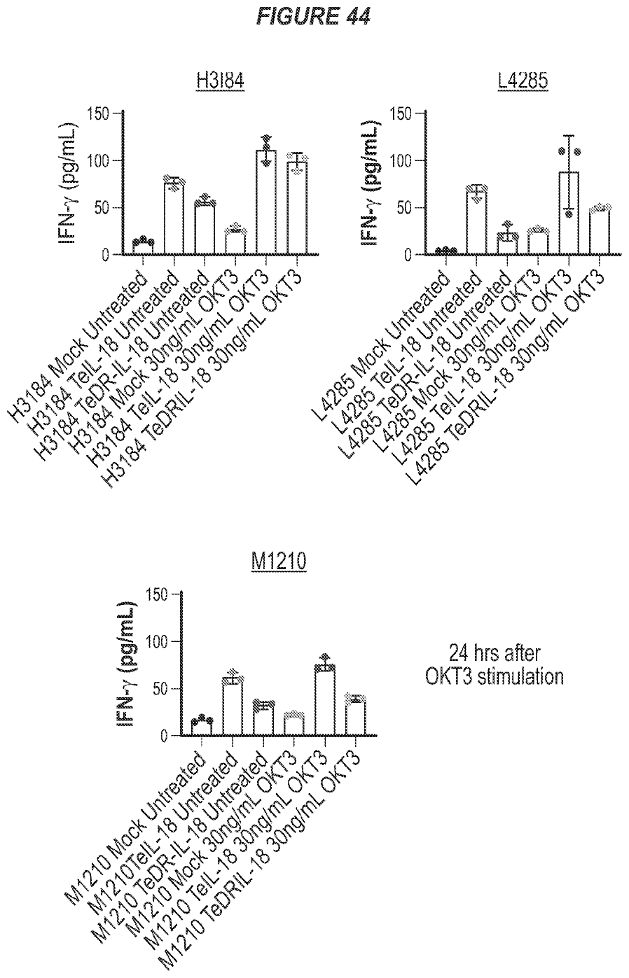

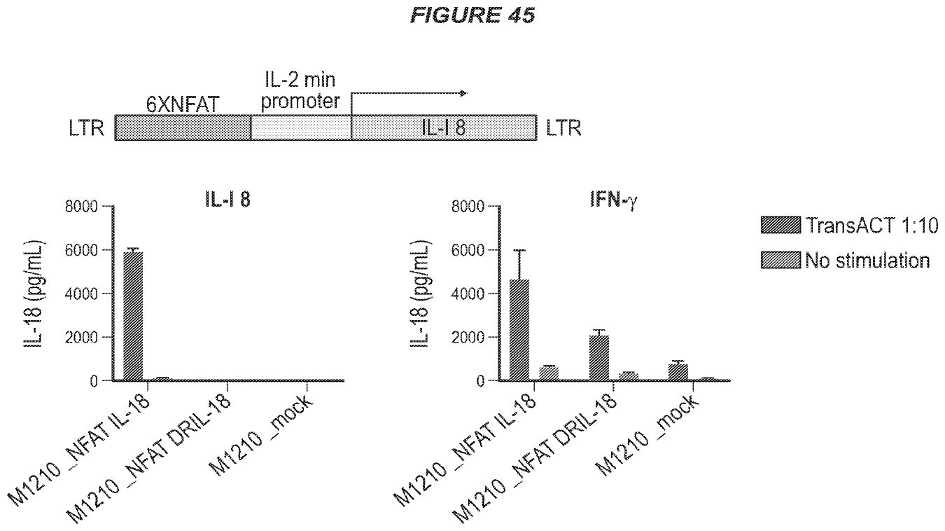

To characterize the functionality of the TeIL-18 and TeDRIL-18 expressed on the modified TILs, three different REP TIL products were engineered to express either TeIL-18 or TeDRIL-18 using the REP methods provided herein. The resulting modified TILs were assessed for IFN-γ production with and without TCR stimulation using anti-CD3 antibody OKT3. As shown in FIG. 44, TeIL-18 REP TILs produced low amounts of IFN-γ without OKT3 stimulation, and increased IFN-γ upon TCR stimulation.

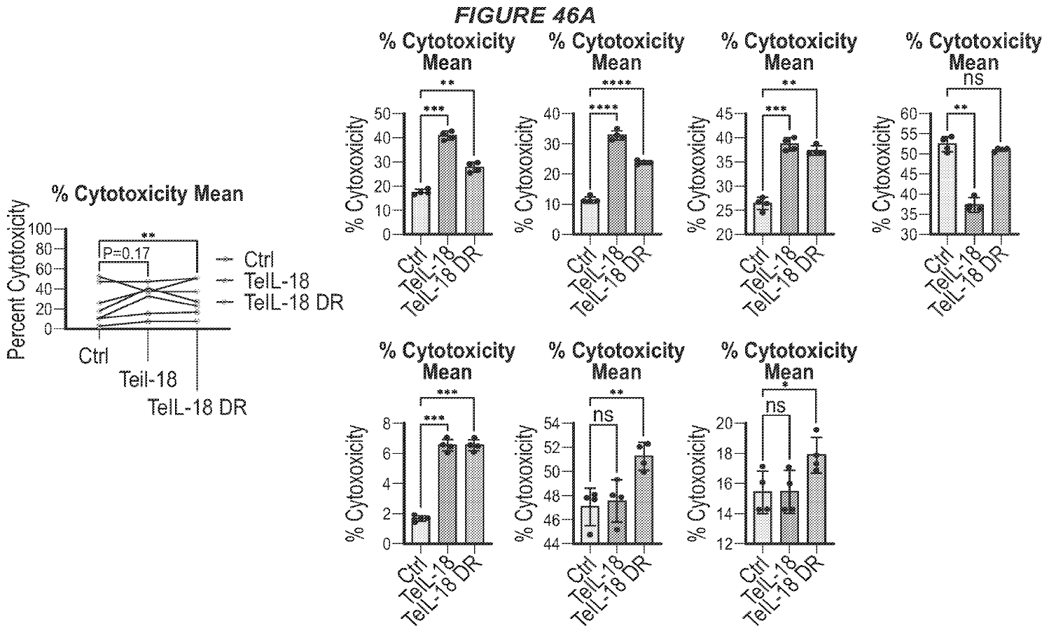

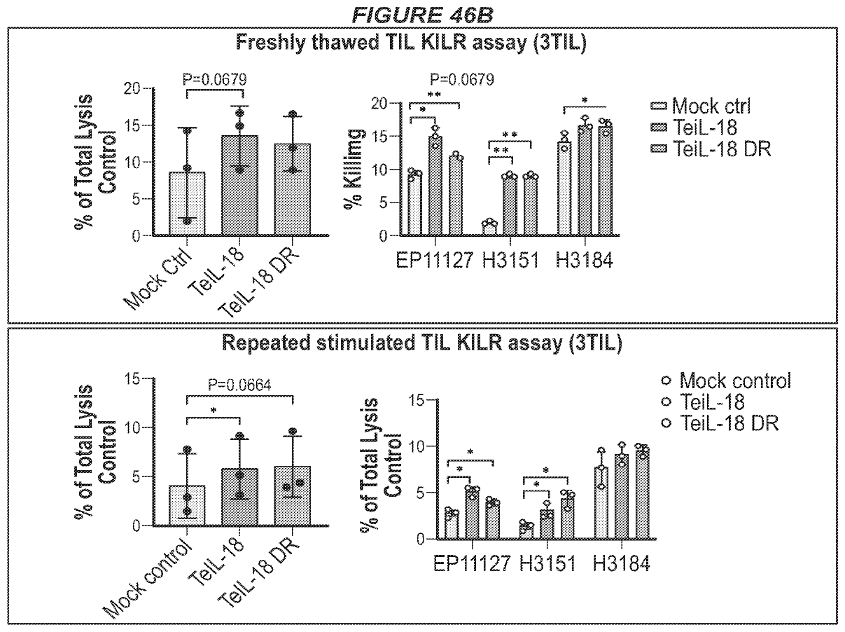

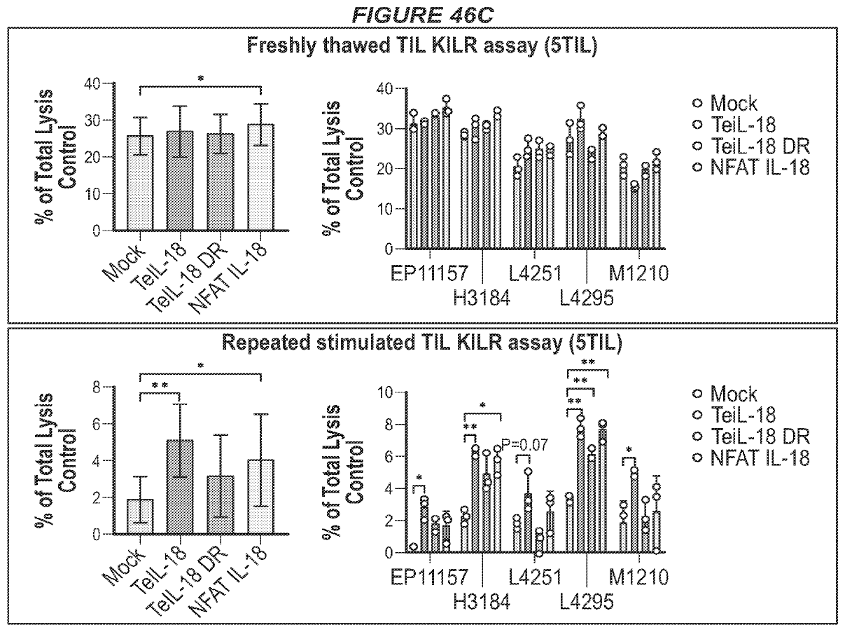

To allow for more controlled expression of IL-18, TILs were modified to express TeIL-18 and TeDRIL-18 under the control of an inducible NFAT promoter. As shown in FIG. 46, TeIL-18 REP TILs exhibited significant levels of IL-18 production after T cell stimulation. Moreover, both TeIL-18 and TeDRIL-18 REP TILs exhibited varying levels of increased IFN-γ production after T cell stimulation as compared to control.

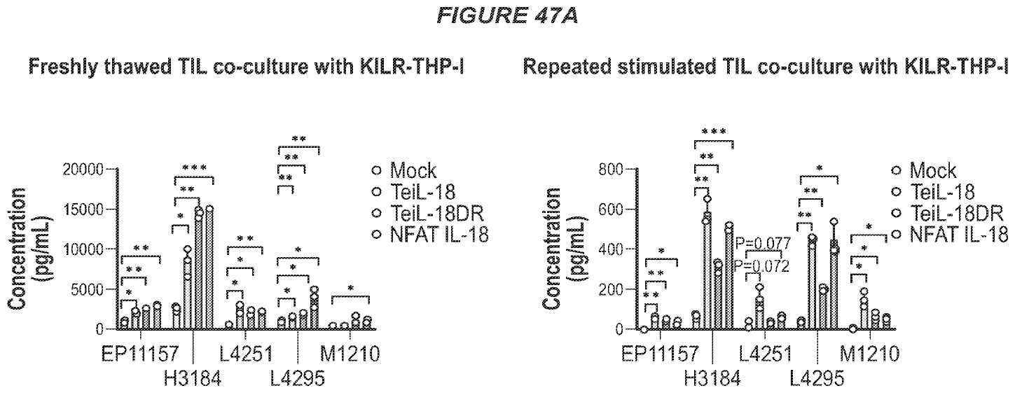

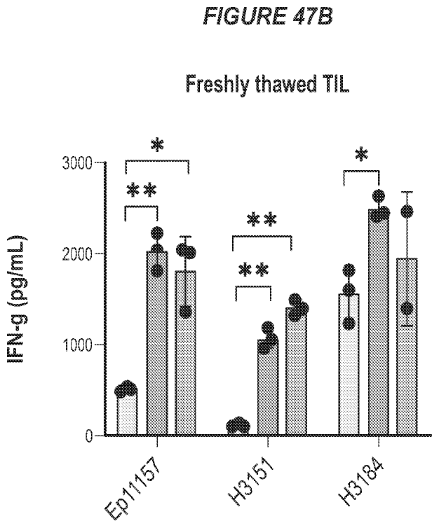

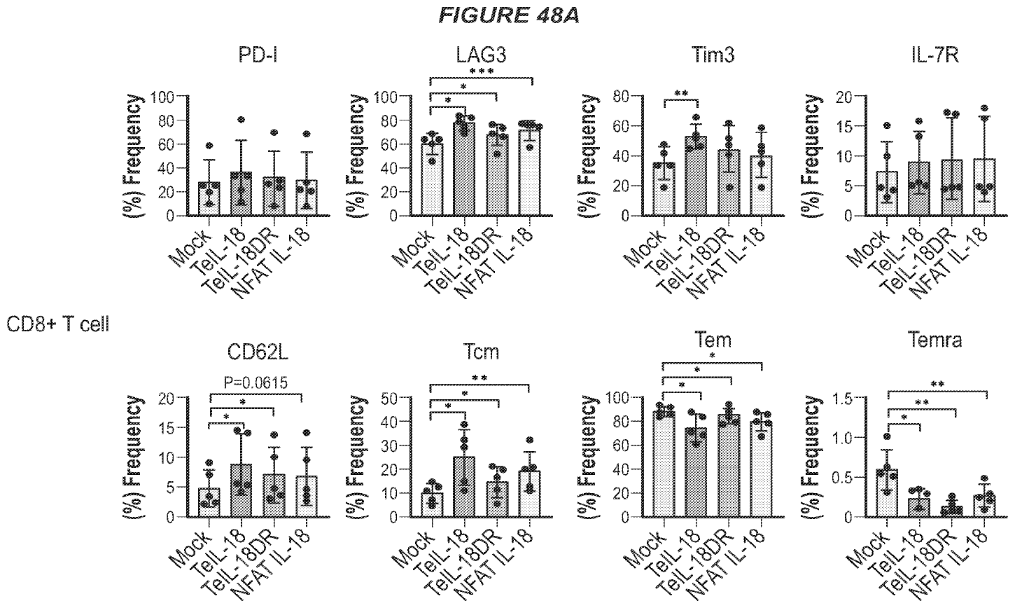

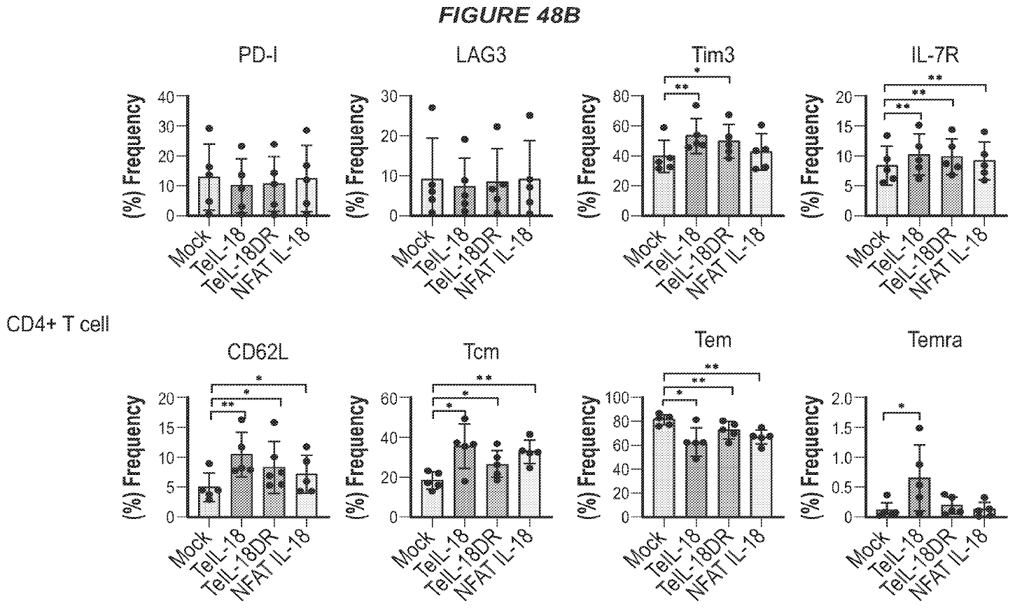

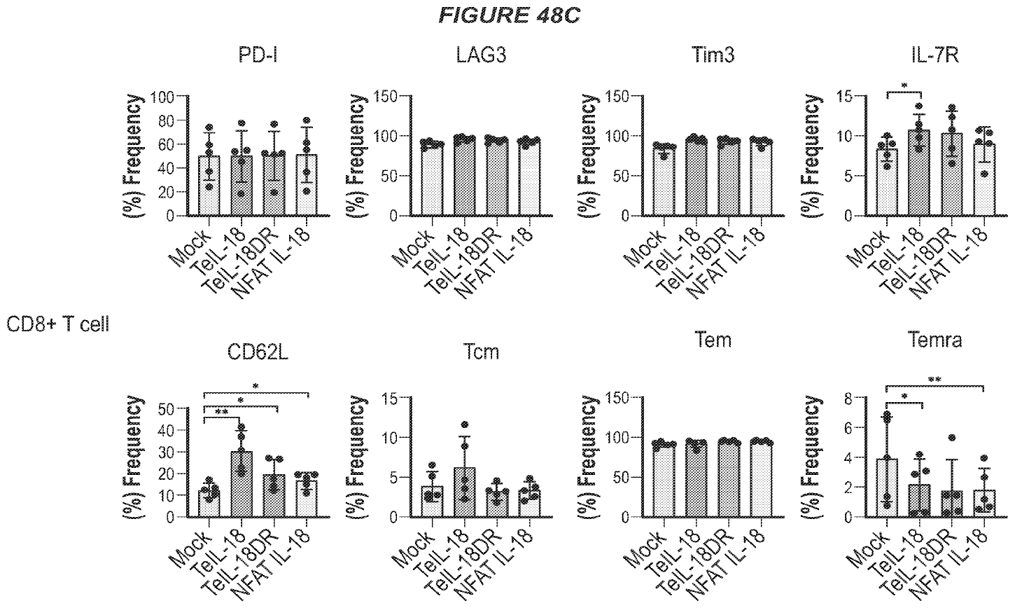

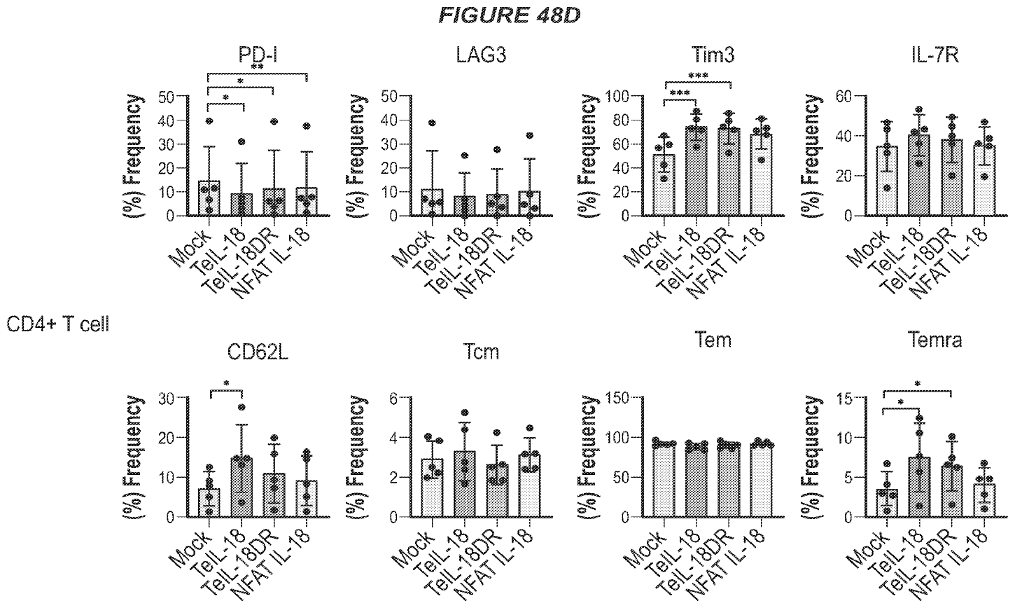

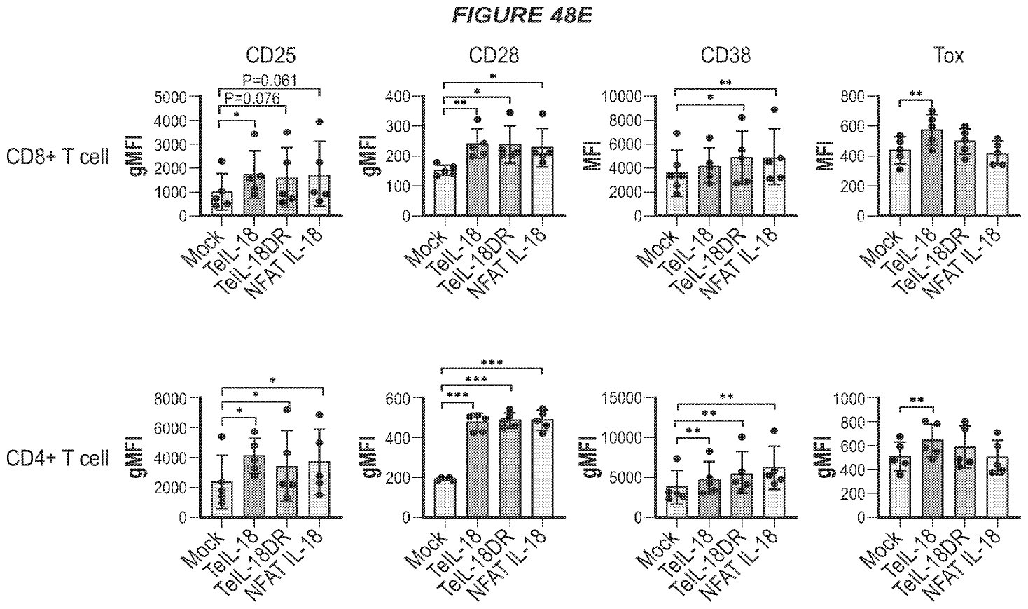

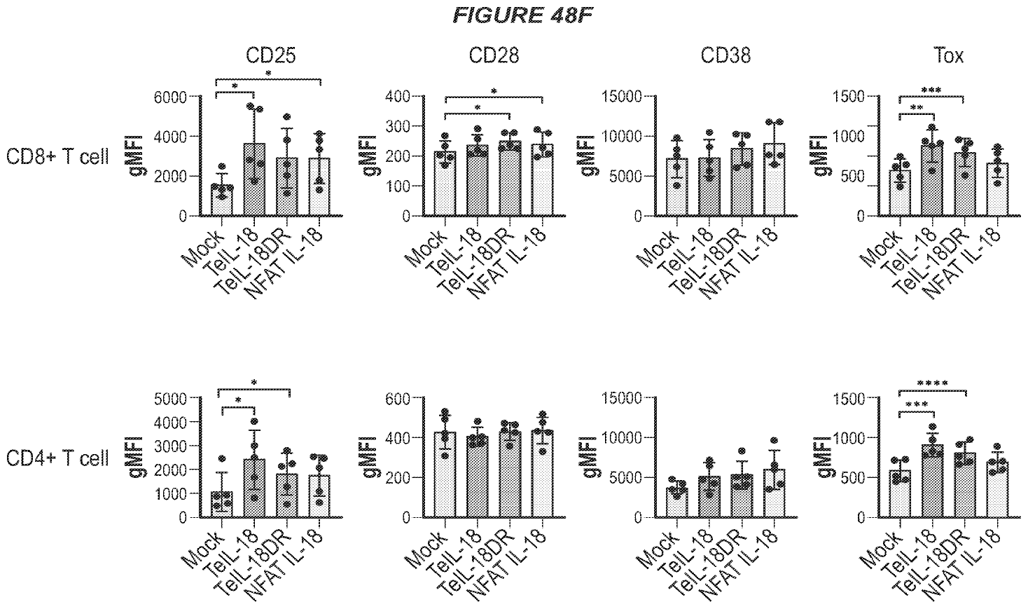

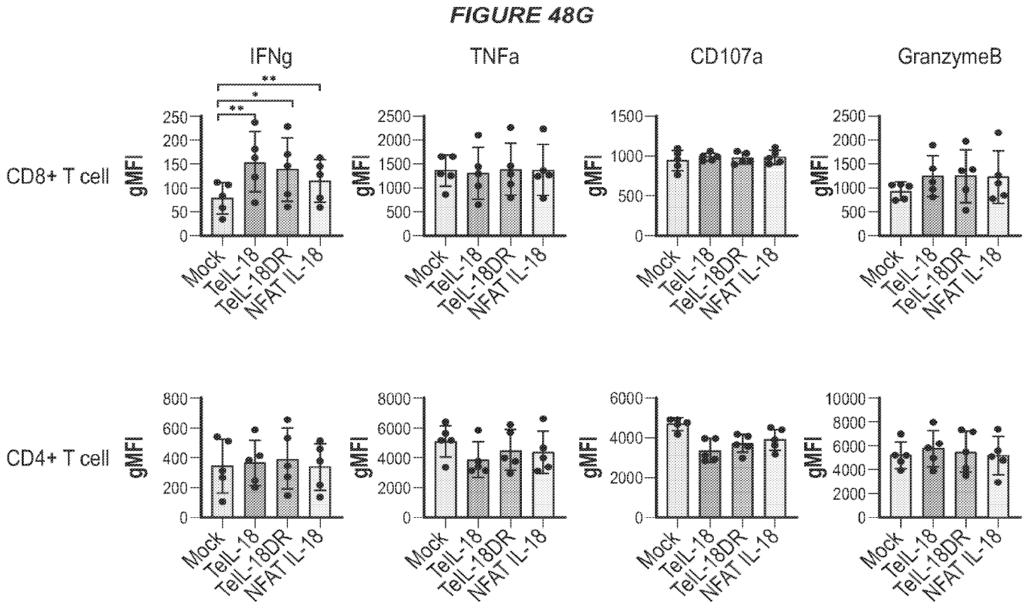

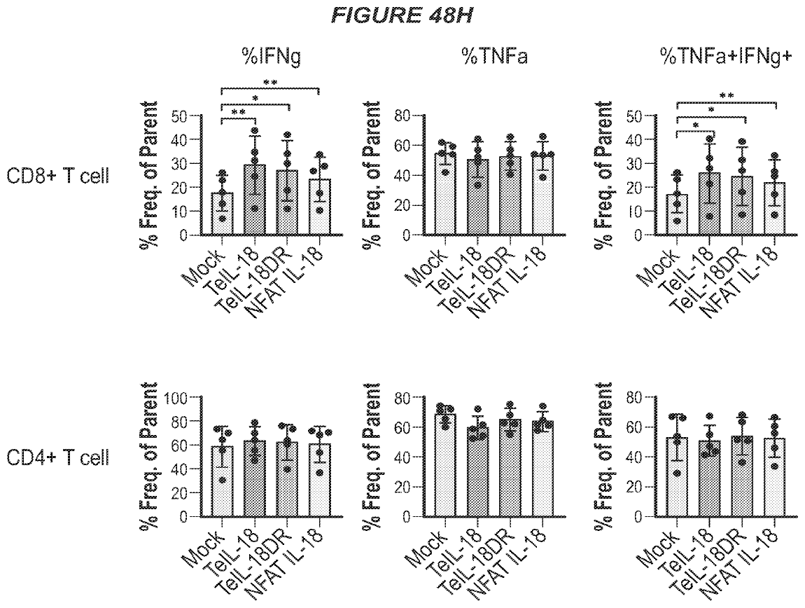

The TeIL-18 and TeDRIL-18 REP TILs were further assessed for functionality using a KILR-THP-I cytotoxicity assay, and phenotype. These experiments were carried out using freshly thawed and repeated stimulated TeIL-18 and TeDRIL-18 REP TILs. For the repeated stimulated experiments, TILs were activated for 3 rounds (Day 0, Day 3, and Day 6) with TransACT (1:100) and harvested on Day 8 for testing. TeIL-18 and TeDRIL-18 REP TILs (FIGS. 46A-46C) showed improved cytotoxicity, and increased IFN-γ production (FIGS. 47A and B, and FIGS. 48G and H). Repeat stimulated TeIL-18 and TeDRIL-18 REP TILs exhibited greater cytotoxicity compared to freshly thawed TILs (See FIG. 46C), perhaps due to an increased population of central memory T cells. Phenotypic assessment for activation in these TIL populations showed higher expression of activation markers (e.g., CD25, TIM3, CD38) in CD4+ and CD8+ subsets for both TeIL-18 and TeDRIL-18 REP TILs. See FIGS. 48E and 48F. TeIL-18 and TeDRIL-18 REP TILs also exhibited less differentiation, with higher expression of T cell stemness biomarkers (e.g., IL-7R, CD62L, and CD28), and a high frequency of central memory T cells (Tcm). See FIG. 48A-48D.

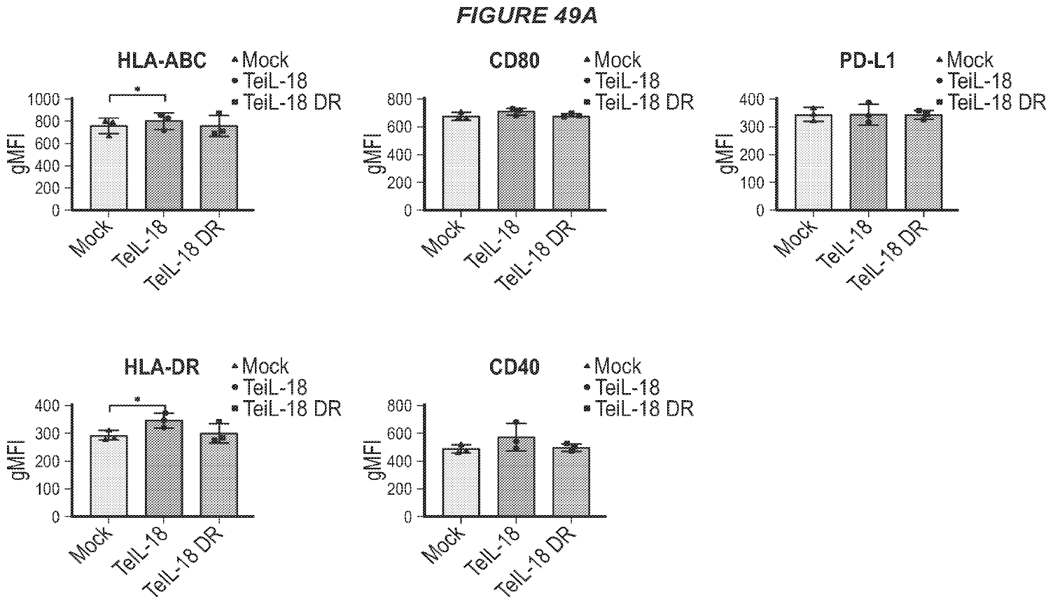

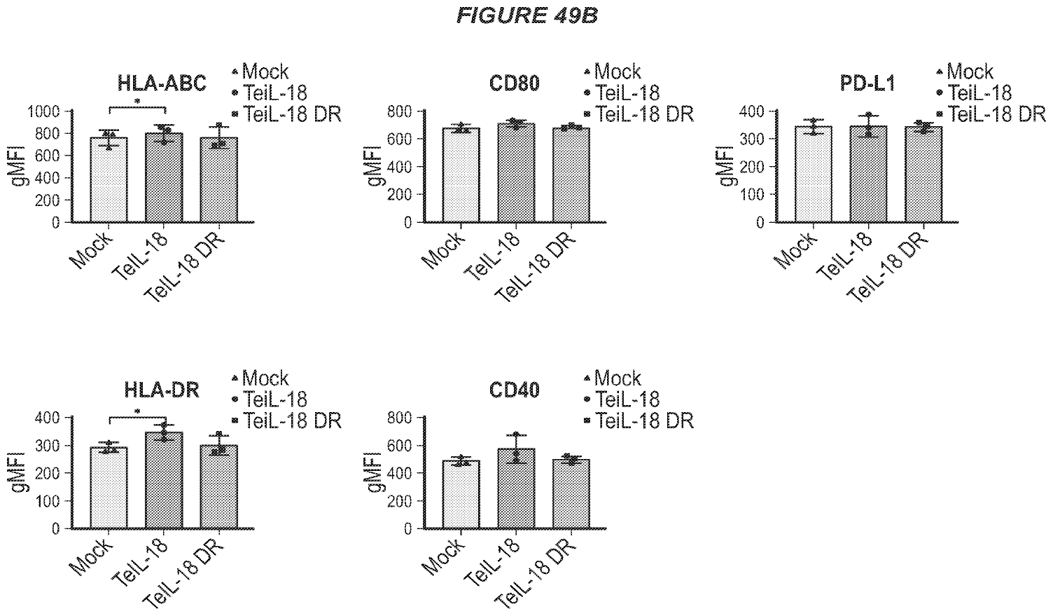

The TeIL-18 and TeDRIL-18 REP TILs were further assessed to determine any effects on THP-I MHC-I and MHC-II expression. The TILs and THP-I cells (10:1) were co-cultured, and THP-I cells were assessed for antigen presentation gene expression. As shown in FIGS. 49A and B, MHC-I and MHC-II were upregulated in THP-I cells in the coculture system.

### Example 15: Production and Characterization of Modified Tils that Expressed Tethered IL-12 Under the Control of an NFAT Promoter and Reduced Expression of PD-1

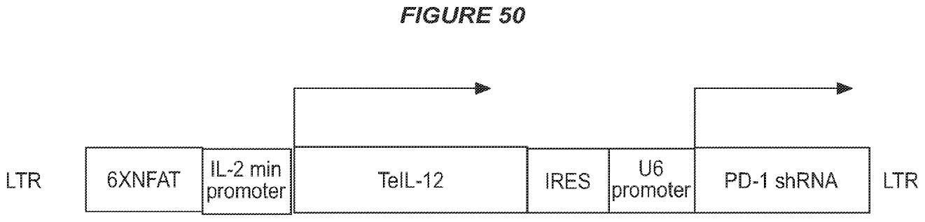

For this study, modified TILs with membrane anchored IL-12 immunodulatory fusion proteins and one or more shRNAs for reducing endogenous PD-1 expression will be made. FIG. 50 depicts an exemplary nucleic acid construct that allows for expression of the member anchored IL-12 (TeIL-12) and PD-1 shRNA. As shown in FIG. 50, expression of the membrane anchored immunodoluatory IL-12 is controlled using an NFAT promoter that includes NFAT responsive elements linked to a minimal human IL-2 promoter, see, e.g., Table 60. The shRNA is under the control of a PolIII promoter (U6).

### Virus Preparation and T Cell Transduction

To make lentivirus, pLenti-vectors containing the tethered cytokine gene sequence (Table 60) and PD-1 shRNA (Table 61) and packaging helper vectors (BaEV-TR, Gag/Pol) will be co-transfected into 293T cells. Lentivirus supernatant will be collected from day 2-3 culture supernatants followed by ultracentrifuge (120,000 g) to concentrate lentivirus for TIL transduction. 1E5 pre-REP cells will be added into Retronectin pre-coated 48 well plates with concentrated lentivirus. Two days after gene transduction, pre-REP cells will be harvested for REP process according to the methods described herein or further phenotypic characterization and functional assays.

TABLE 60
Sequences for TeIL-12/shRNA constructs

| Construct | DNA sequence |
|---|---|
| 6XNFAT | GGAGGAAAAACTGTTTCATACAGAAGGCGTCAATTAGGAGGAAAAACTG |
| binding | TTTCATACAGAAGGCGTCAATTAGGAGGAAAAACTGTTTCATACAGAAG |
| motif | GCGTCAATTGGTCCCATCGAATTAGGAGGAAAAACTGTTTCATACAGAA |
| | GGCGTCAATTAGGAGGAAAAACTGTTTCATACAGAAGGCGTCAATTAGG |
| | AGGAAAAACTGTTTCATACAGAAGGCGTCAATTGGTCCCGGGA (SEQ ID |
| | NO: 390) |
| IL-2min | CATTTTGACACCCCCATAATATTTTTCCAGAATTAACAGTATAAATTGCA |
| promoter | TCTCTTGTTCAAGAGTTCCCTATCACTCTCTTTAATCACTACTCACAGTAA |
| | CCTCAACTCCTG ((SEQ ID NO: 391) |
| TeIL-12 | ATGGATTGGACCTGGATTCTGTTCCTCGTGGCCGCCGCTACCCGCGTGCA |
| | CTCCATCTGGGAGCTGAAGAAAGACGTGTACGTGGTGGAATTGGATTGG |
| | TACCCCGACGCCCCAGGCGAGATGGTGGTTCTGACTTGCGACACGCCGG |
| | AGGAGGACGGCATCACCTGGACGCTGGACCAGAGCAGCGAAGTGCTGG |
| | GCTCTGGTAAGACCTTAACCATCCAGGTCAAGGAGTTCGGTGATGCAGG |
| | ACAGTACACGTGTCACAAGGGCGGGGAGGTCTTGTCCCATAGCCTGCTG |
| | CTCCTGCACAAGAAGGAAGACGGCATCTGGTCTACGGATATCCTTAAAG |
| | ACCAGAAGGAGCCCAAGAACAAAACCTTCCTGCGATGCGAGGCTAAGA |
| | ATTACTCCGGGCGCTTTACCTGTTGGTGGCTCACAACCATCTCGACCGAC |
| | CTGACCTTCTCCGTGAAGTCTTCCAGGGGATCTTCTGATCCGCAAGGCGT |
| | TACCTGCGGGGCCGCTACCCTGTCGGCTGAGCGCGTCCGTGGTGACAAC |
| | AAGGAGTACGAGTACTCGGTGGAATGCCAGGAGGACAGCGCCTGCCCAG |
| | CGGCCGAAGAGTCCTTGCCCATCGAGGTGATGGTGGACGCTGTACACAA |
| | GCTCAAGTACGAGAACTACACCTCCTCCTTCTTTATACGCGACATCATCA |
| | AGCCTGACCCCCCTAAAAACCTGCAGCTGAAGCCCCTTAAAAACTCTCG |
| | ACAGGTCGAGGTATCCTGGGAGTACCCCGACACCTGGTCTACTCCTCATT |
| | CGTATTTCAGCCTGACCTTCTGCGTGCAGGTGCAGGGCAAGTCCAAGCGC |
| | GAGAAGAAGGACCGCGTGTTCACCGACAAAACCAGCGCGACTGTCATTT |
| | GTCGGAAGAACGCGAGCATCTCCGTACGGGCACAGGATCGCTATTACTC |
| | CAGCAGTTGGTCAGAGTGGGCTTCGGTTCCGTGTTCTGGAGGAGGTGGC |
| | GGCGGCTCACGCAACCTGCCCGTGGCGACGCCGGACCCGGGTATGTTTC |
| | CTTGTCTGCACCACTCTCAGAACTTGCTCCGTGCCGTCTCGAATATGCTG |
| | CAGAAGGCCCGCCAGACTCTGGAGTTCTATCCATGCACTAGTGAGGAGA |
| | TCGATCATGAGGACATCACCAAGGACAAGACATCCACCGTGGAGGCTTG |
| | TCTGCCGCTGGAGTTGACAAAGAACGAGAGCTGCCTCAATAGCCGCGAG |
| | ACTTCCTTCATCACCAACGGCTCCTGCCTCGCGAGCCGTAAGACCTCCTT |
| | TATGATGGCGCTGTGCCTGTCAAGTATCTACGAAGATCTGAAAATGTATC |
| | AGGTGGAGTTCAAAACTATGAACGCAAAACTCCTGATGGATCCCAAGCG |
| | CCAAATTTTCTTGGACCAGAACATGCTTGCCGTGATCGACGAGCTGATGC |
| | AGGCCCTGAACTTCAACTCCGAGACCGTCCCTCAGAAGTCGTCTCTGGAG |
| | GAACCCGACTTTTACAAGACAAAGATCAAGCTGTGCATCCTGCTACACG |
| | CGTTCCGCATTCGGGCCGTGACCATTGACCGCGTGATGTCCTACCTGAAT |
| | GCCTCGTCCGGCGGGGGGGGCTCCGGTGGGGGCGGCTCTGGCGGAGGCG |
| | GCTCAGGTGGCGGCGGCTCCGGCGGAGGTTCGCTTCAGCTTCTGCCCAGT |
| | TGGGCAATCACTCTGATTTCCGTCAACGGAATCTTCGTCATCTGCTGCCT |
| | GACCTACTGCTTTGCTCCCAGATGTAGAGAGCGCCGCCGCAACGAGCGC |
| | CTGCGTCGTGAGAGCGTGAGGCCTGTGTGA (SEQ ID NO: 392) |
| IRES | CCCCTCTCCCTCCCCCCCCCCTAACGTTACTGGCCGAAGCCGCTTGGAAT |
| | AAGGCCGGTGTGCGTTTGTCTATATGTTATTTTCCACCATATTGCCGTCTT |
| | TTGGCAATGTGAGGGCCCGGAAACCTGGCCCTGTCTTCTTGACGAGCATT |
| | CCTAGGGGTCTTTCCCCTCTCGCCAAAGGAATGCAAGGTCTGTTGAATGT |
| | CGTGAAGGAAGCAGTTCCTCTGGAAGCTTCTTGAAGACAAACAACGTCT |
| | GTAGCGACCCTTTGCAGGCAGCGGAACCCCCCACCTGGCGACAGGTGCC |
| | TCTGCGGCCAAAAGCCACGTGTATAAGATACACCTGCAAAGGCGGCACA |
| | ACCCCAGTGCCACGTTGTGAGTTGGATAGTTGTGGAAAGAGTCAAATGG |
| | CTCTCCTCAAGCGTATTCAACAAGGGGCTGAAGGATGCCCAGAAGGTAC |
| | CCCATTGTATGGGATCTGATCTGGGGCCTCGGTGCACATGCTTTACATGT |
| | GTTTAGTCGAGGTTAAAAAAACGTCTAGGCCCCCCGAACCACGGGGACG |
| | TGGTTTTCCTTTGAAAAACACGATGATAA (SEQ ID NO: 393) |
| U6 | GAGGGCCTATTTCCCATGATTCCTTCATATTTGCATATACGATACAAGGC |
| promoter | TGTTAGAGAGATAATTGGAATTAATTTGACTGTAAACACAAAGATATTA |
| | GTACAAAATACGTGACGTAGAAAGTAATAATTTCTTGGGTAGTTTGCAGT |
| | TTTAAAATTATGTTTTAAAATGGACTATCATATGCTTACCGTAACTTGAA |
| | AGTATTTCGATTTCTTGGCTTTATATATCTTGTGGAAAGGACGAAACACC |
| | (SEQ ID NO: 394) |

TABLE 61
PD-1 shRNA sequences

| Construct | DNA sequence |
|---|---|
| PD-1 | GGATTTCCAGTGGCGAGAGAACTCGAGTTCTCTCGCCACTGGAAATCC (SEQ |
| shRNA #1 | ID NO: 395) |
| PD-1 | CATTGTCTTTCCTAGCGGAATCTCGAGATTCCGCTAGGAAAGACAATG (SEQ ID |
| shRNA #2 | NO: 396) |
| PD-1 | CAAGGCGCAGATCAAAGAGAGCTCGAGCTCTCTTTGATCTGCGCCTTG (SEQ |
| shRNA #3 | ID NO: 397) |
| PD-1 | AAGGCGCAGATCAAAGAGAGCCTCGAGGCTCTCTTTGATCTGCGCCTT (SEQ |
| shRNA #4 | ID NO: 398) |
| PD-1 | ATCGGAGAGCTTCGTGCTAAACTCGAGTTTAGCACGAAGCTCTCCGAT (SEQ |
| shRNA #5 | ID NO: 399) |
| PD-1 | AGTATGCCACCATTGTCTTTCCTCGAGGAAAGACAATGGTGGCATACT (SEQ ID |
| shRNA #6 | NO: 400) |
| PD-1 | GGATTTCCAGTGGCGAGAGAACTCGAGTTCTCTCGCCACTGGAAATCC (SEQ |
| shRNA #7 | ID NO: 401) |

### Flow Cytometry

TIL TeIL-12 and PD-1 expression will be assessed using immunofluorescence staining and flow cytometric analysis.

### T Cell Count and Viability

REP cells will be harvested after TIL expansion. Live cell numbers will be assessed by AOPI assay using Cellometer K2 cell counter machine (Nexcelom, Lawrence, MA).

### Pre-REP and REP Process

Human tumor samples will be dissected into −3 mm pieces and cultured in Grex10 with recommended culture medium containing IL-2 (6000 IU/ml). After 11 days of culture, pre-REP cells will be harvested for 2-day activation and 2-day lentiviral transduction. Transduced Pre-REP cells will be then propagated in a REP expansion process with irradiated PBMCs, anti-CD3 antibody and 3,000 IU/ml of IL-2 for another 11 days.

### Example 16: Production and Characterization of Modified Tils that Expressed Tethered IL-12 Under the Control of an NFAT Promoter

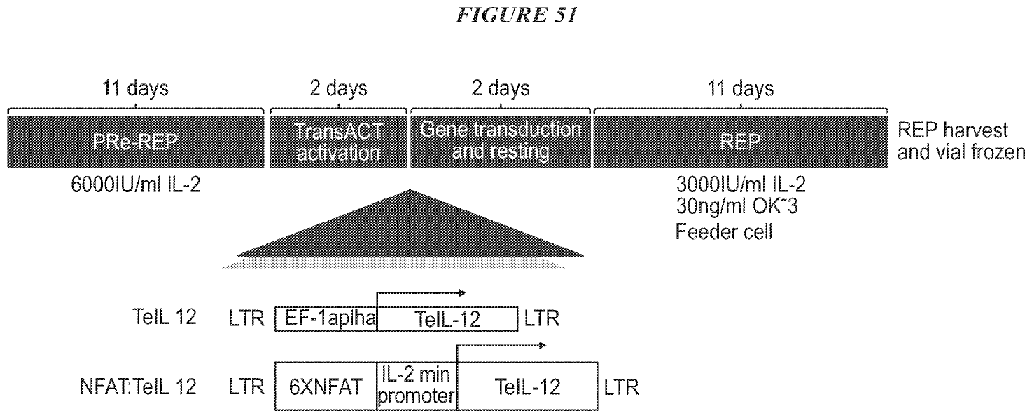

FIG. 51 shows an exemplary TIL expansion process including transduction of a lentiviral vector encoding TeIL-12 controlled by either an EF-1alpha promotor or an NFAT promoter that includes NFAT responsive elements linked to a minimal human IL-2 promoter.

After 11 days of pre-REP in cell culture media including 6000 IU/mL IL-2, TILs were stimulated for 2 days with TransACT at a ratio of 1:100, in the presence of 10 ng/mL IL-7, and 20 ng/mL IL-15.

Spin transduction was performed with Lentivirus+ at 1:100 Lentiboost-P in CM2+IL-7 and IL-15 in a RetroNectin® coated plate. After the transduction, TILs were then rested for 2 days, followed with a REP process with 25K TILs, 5E6 Feeder cells in CM2+IL-2 at 3000 IU/mL+OKT3 at 30 ng/mL (total vol 4 mL) in GREX-24. After 5 days of incubation, an additional 4 mL of CM2+3000 IU/mL IL-2 was added to each well. Incubation continued for a total of 11 days followed by TIL harvest.

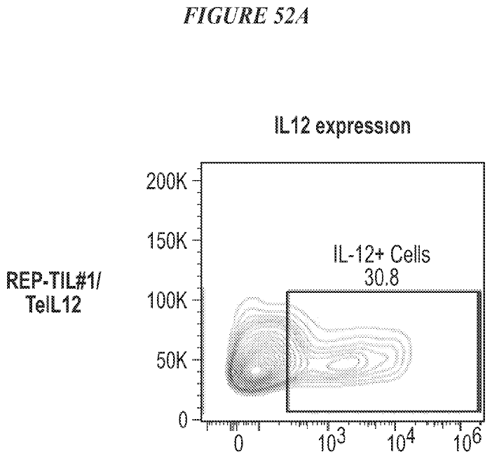

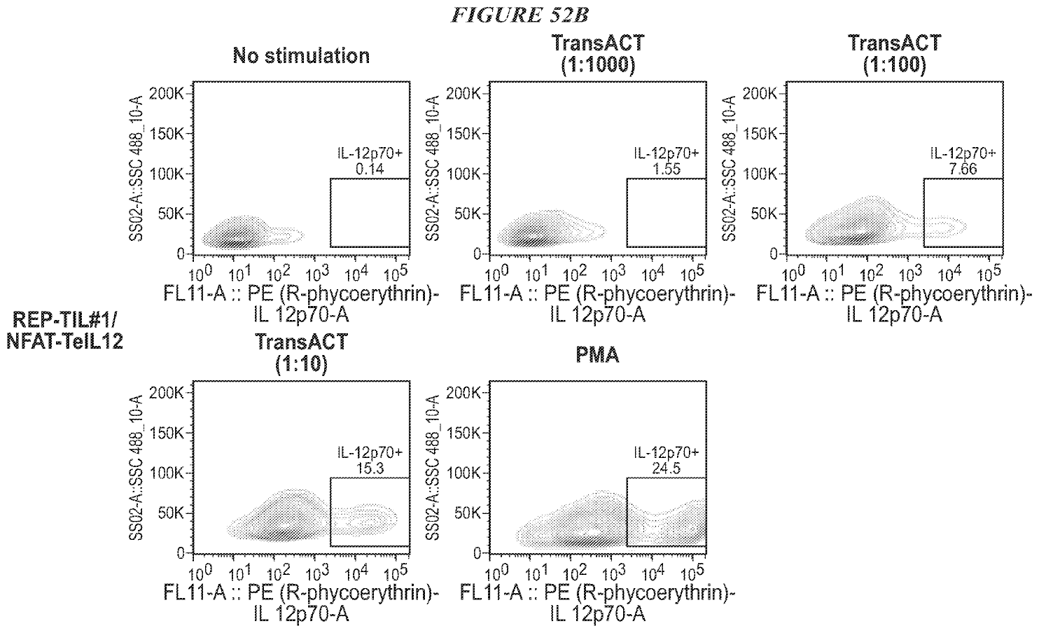

After REP harvest, surface expression of IL-12 on TILs was examined by flow cytometry with IL-12P70 flow Ab (FIG. 52A). TILs were also stimulated with TransACT or PMA. 48 hours after stimulation, surface expression of IL-12 was examined by flow cytometry (FIG. 52B).

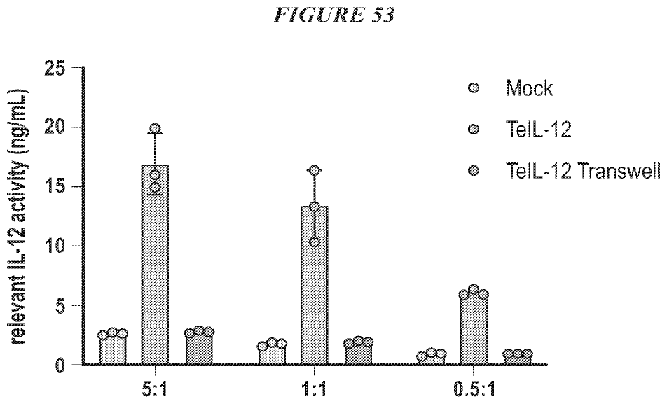

TeIL-12 expressing TILs were also assayed for IL-12 activity (FIG. 53). 5E4 HEK-IL18 reporter cells were seeded in a 96-well plate in DMEM+10% FBS media. After adherence, transduced TILs were seeded at a 5:1, 1:1 and 0.5:1 ratio at 2.5E5, 5E4, and 2.5E4 cells/well, respectively. Transwell was set. After overnight incubation, 20 μl culture supernant was collected and transferred to another 96 well plate. 180 μL of Quantiblue Reagent was added to each well and incubated at 37° C. until the highest IL-12 standard turned into a dark purple color.

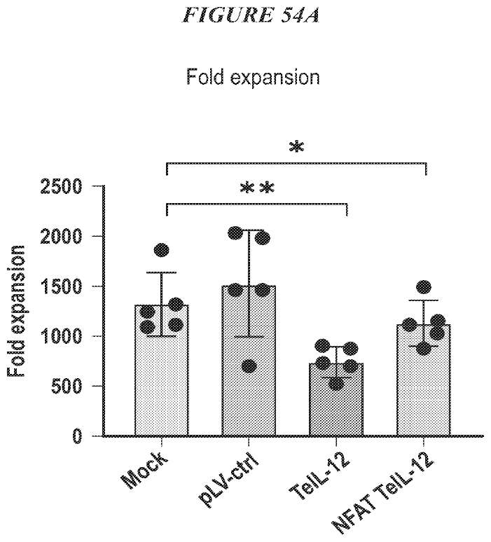

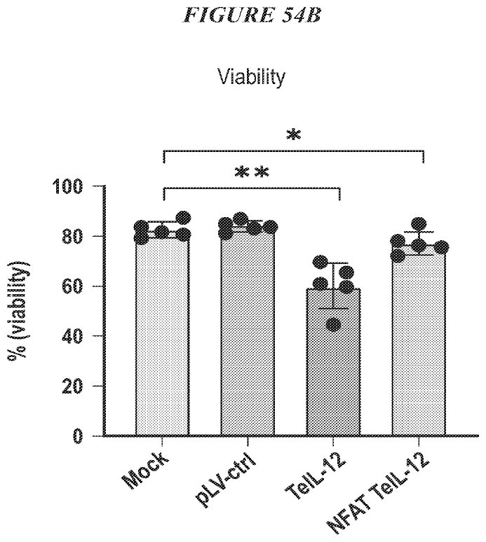

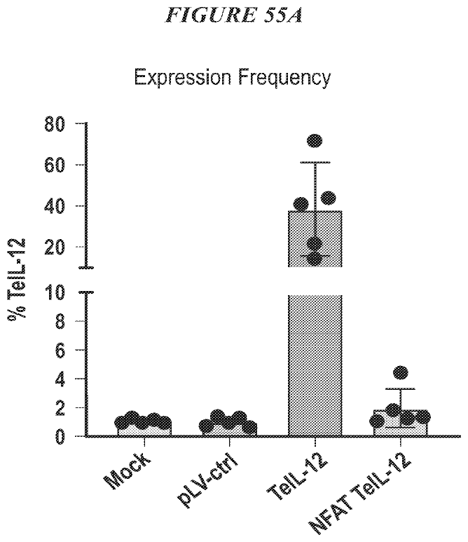

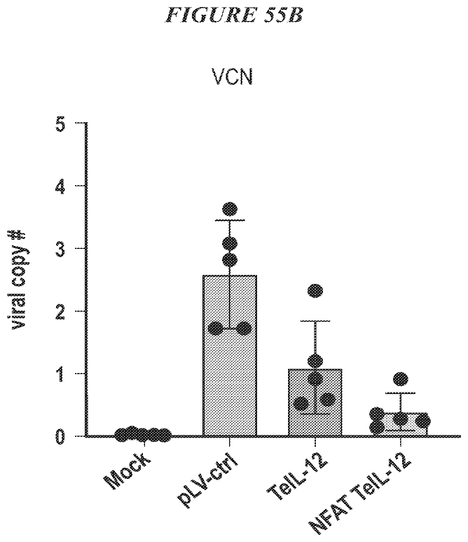

Five tumor samples (2 lung, 1 head & neck, 1 breast, and 1 ovarian) were used to generate TILs for this study. FIG. 54 shows fold expansion (A) and viability (B) of post-REP TILs. FIG. 55 shows expression frequency of TeIL-12 (A) and viral copy number per cell (B) detected by digital PCR.

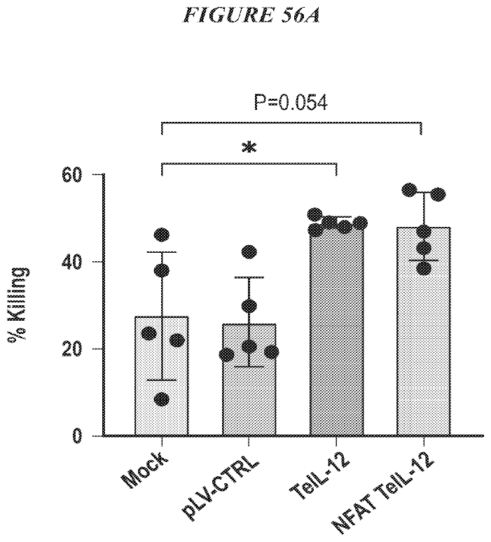

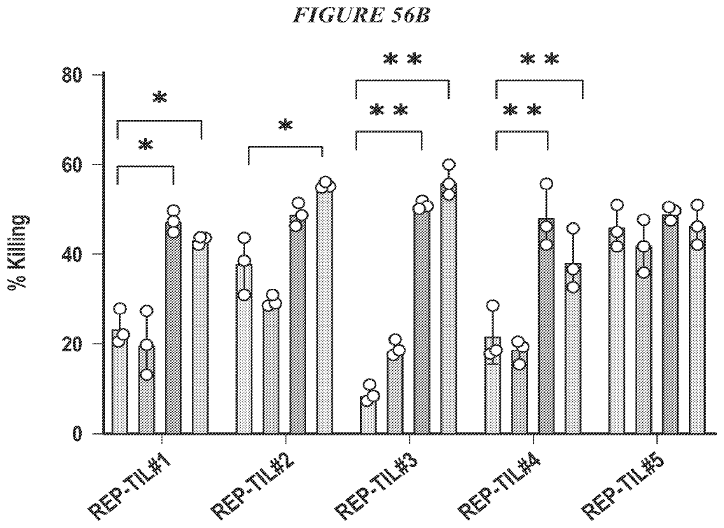

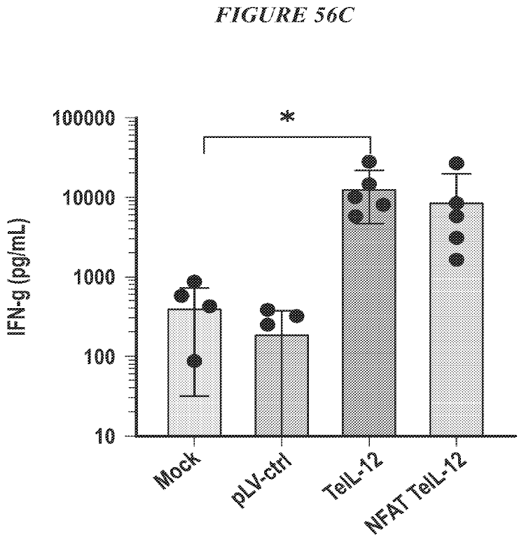

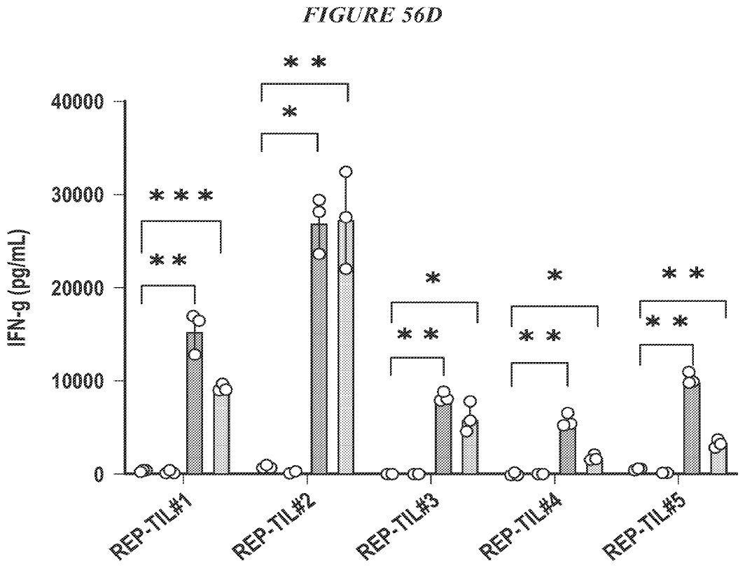

Cytotoxicity of TeIL-12 expressing TILs was assessed by KILR® THP-1 assay. Post-REP-TILs (1E5) and KILR-THP-1 cells (1E4) were co-cultured in 96 well plates in 10:1 ratio. 24 hours after incubation, supernatant was harvested for cytotoxicity assay. THP-1 target cell death was detected using the KILR detection reagent (FIGS. 56A & B). IFN-γ production (FIGS. 56C & D) and IL-12 shedding in culture supernatant were examined by ELISA (FIG. 56E). Both TeIL-12 and NFAT-driven inducible TeIL-12 TILs demonstrated improved killing activity in a THP-1 based allogeneic cytotoxicity assay. >30 folds increase of IFN-γ was detected in the co-culture assay. TeIL-12 shedding happened during the co-culture of TeIL-12 TILs and KILR® THP-1 cells.

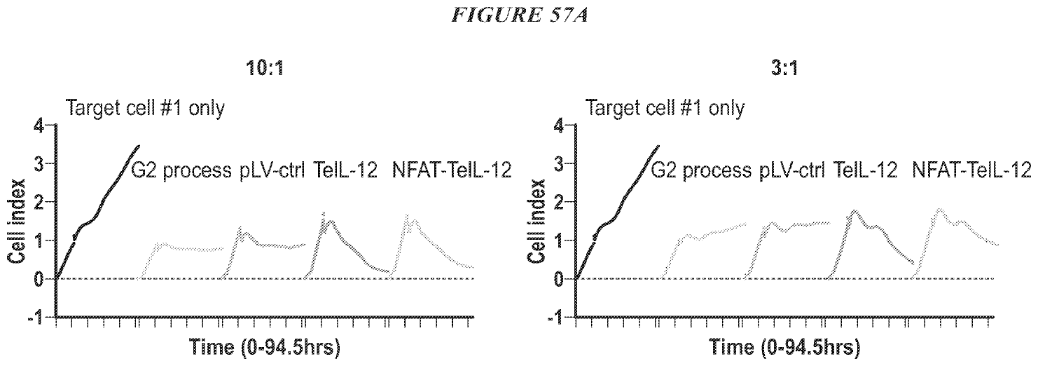

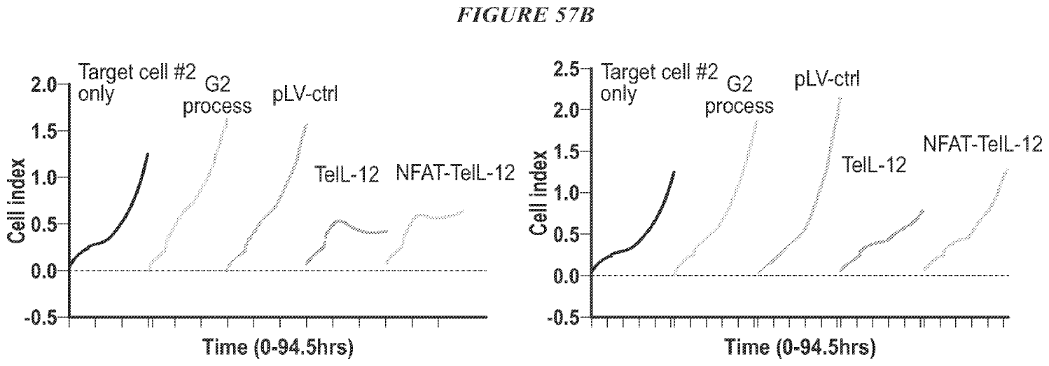

Cytotoxicity of TeIL-12 expressing TILs was also assessed by xCelligence RTCA assay. 10,000 target cells #1 were seeded in an RTCA E-plate. After overnight incubation, post-REP-TILs were added at a 10:1 or 3:1 ratio. Cell growth was dynamically monitored by an xCELLigence RTCA instrument (FIG. 57A). The same assay was performed with target cell #2 (FIG. 57B). TeIL-12 and NFAT TeIL-12 TILs demonstrated superior allogenic cytotoxicity.

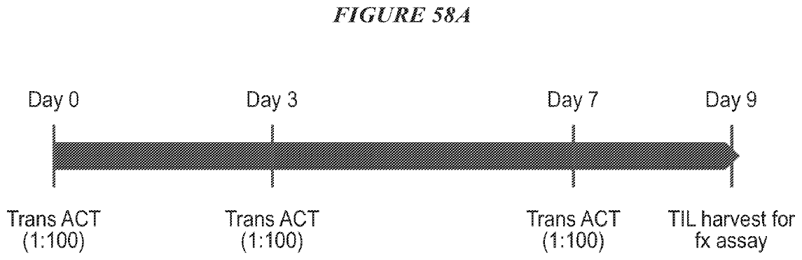

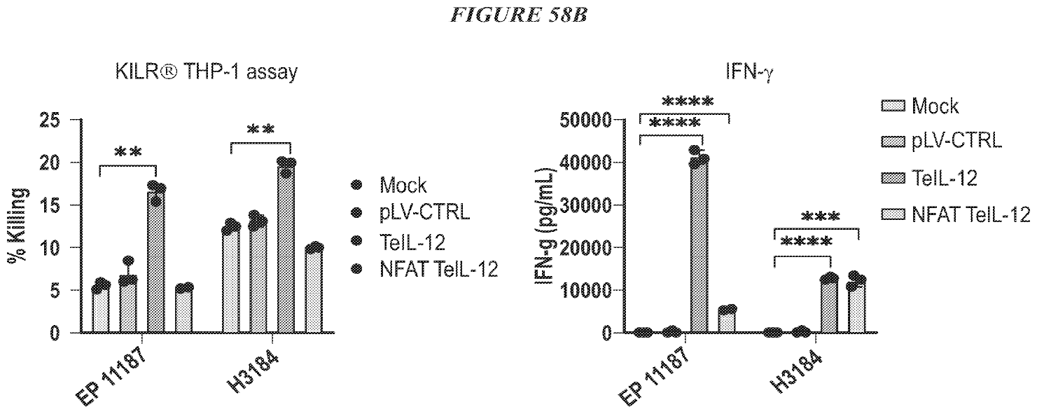

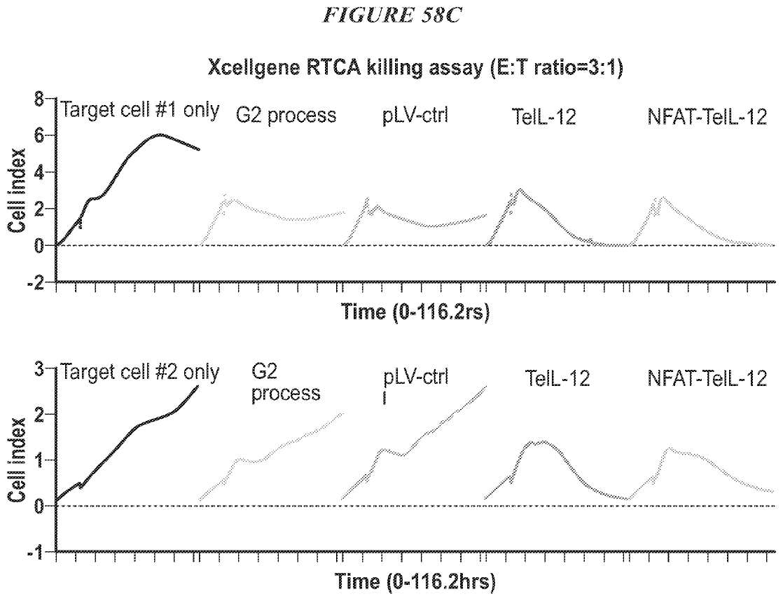

To test serial killing capability, post-REP-TILs were stimulated repeatedly with TransACT (1:100) and harvested 2 days after the last TransACT stimulation (FIG. 58A). KILR® THP-1 cytotoxicity assay, IFN-γ quantification (FIG. 58B), and xCELLigence RTCA killing assay (FIG. 58C) were performed to evaluate TIL killing efficacy.

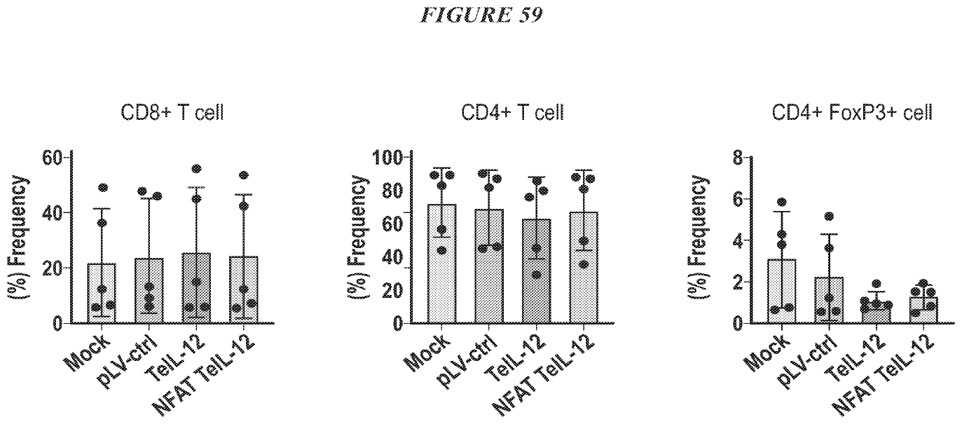

CD8+, CD4+, and CD4+FoxP3+ T cell subsets of post-REP TILs were analyzed by flow cytometry (FIG. 59). No difference was observed on the distribution of CD8+, CD4+, and CD4+FoxP3+ T cell subsets.

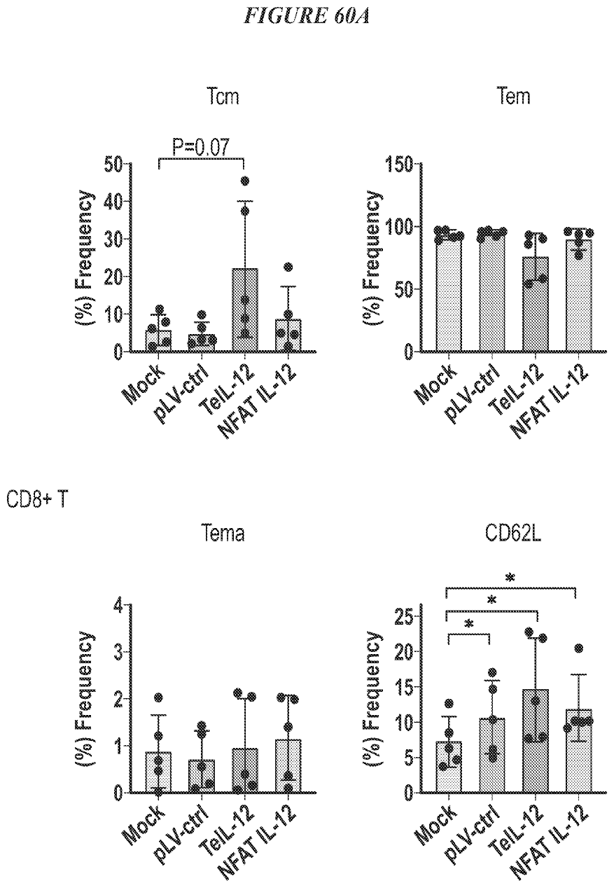

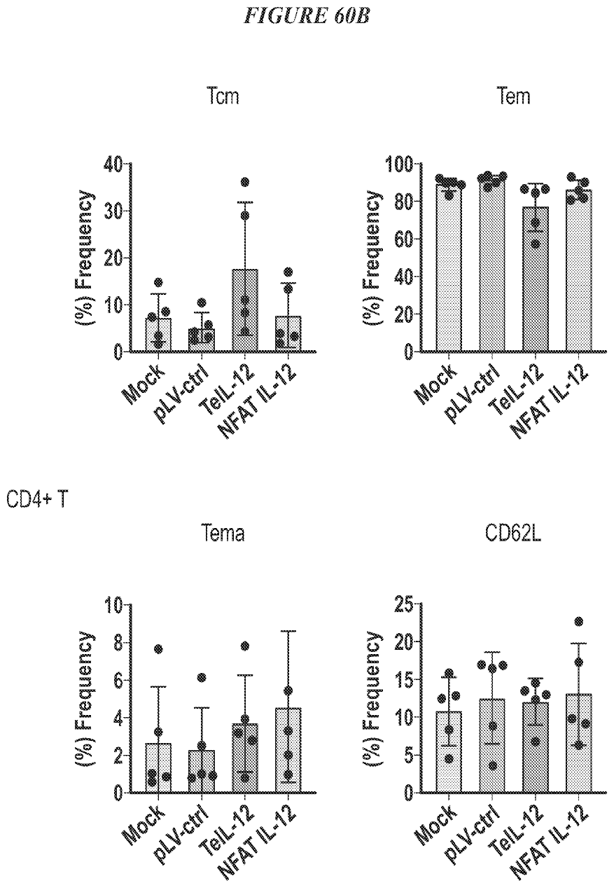

Tcm (CCR7+CCR45RA−), Tem (CCR7−CD45RA−), Tema (CCR7−CD45RA+) subsets and expression of CD62L of post-REP TILs were detected by flow cytometry (FIG. 60). No statistic significant difference was observed in Tcm, Tem and Tema distribution. TeIL-12/NFAT-TeIL-12 post-REP TILs showed less differentiation, with increased expression of CD62L.

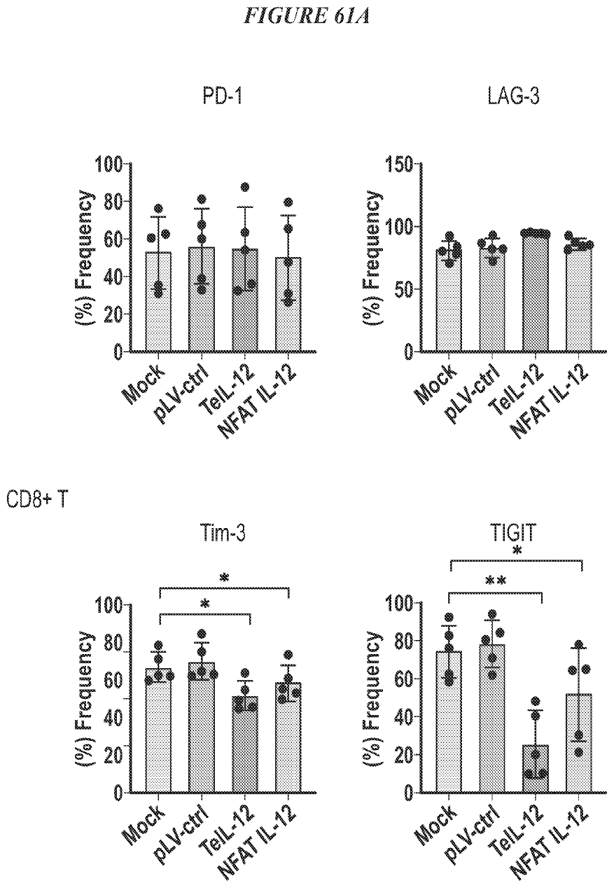

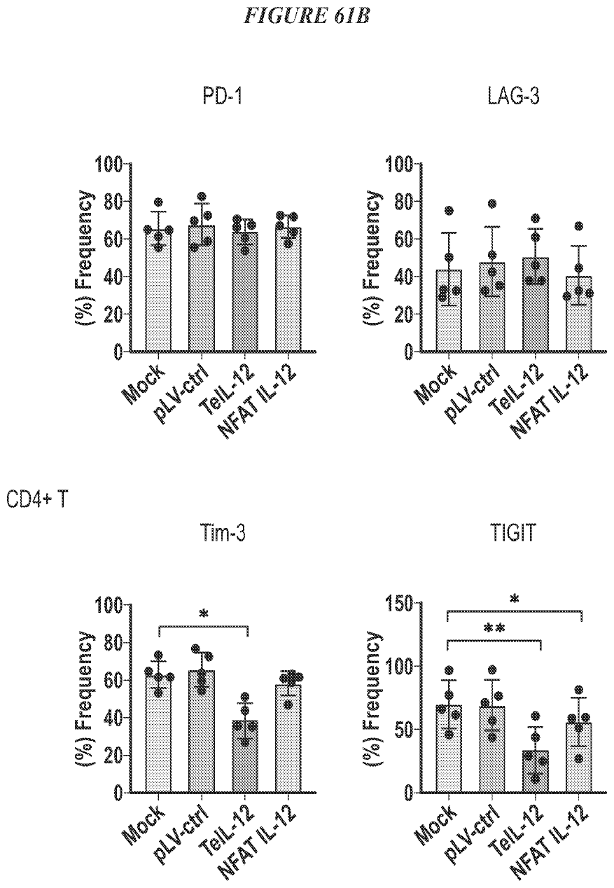

Expression of T cell exhaustion markers PD-1, LAG-3, Tim-3 and TIGIT on post-REP TILs was detected by flow cytometry (FIG. 61). No significant change of PD-1 and LAG-3 expression was observed. Reduced expression of Tim-3 and TIGIT, more profound in TIGIT, was observed.

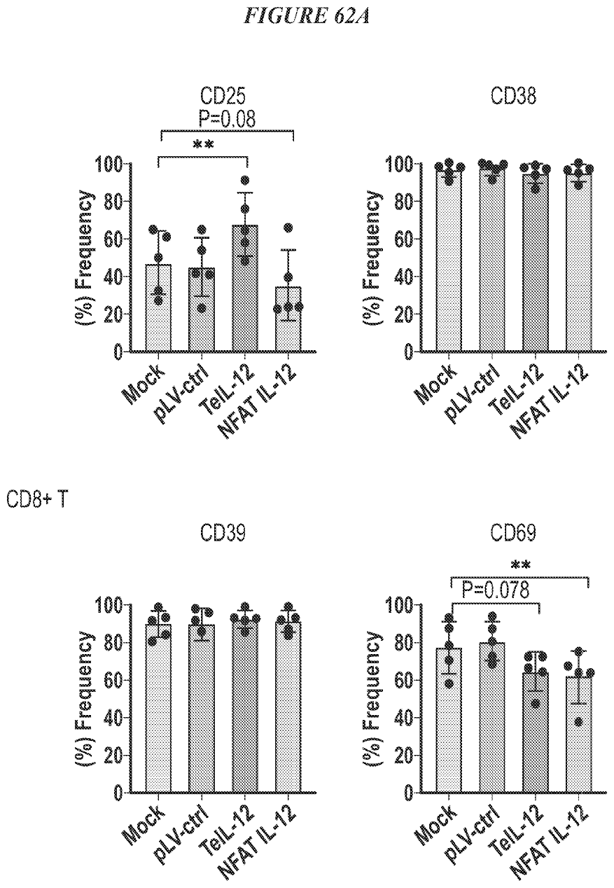

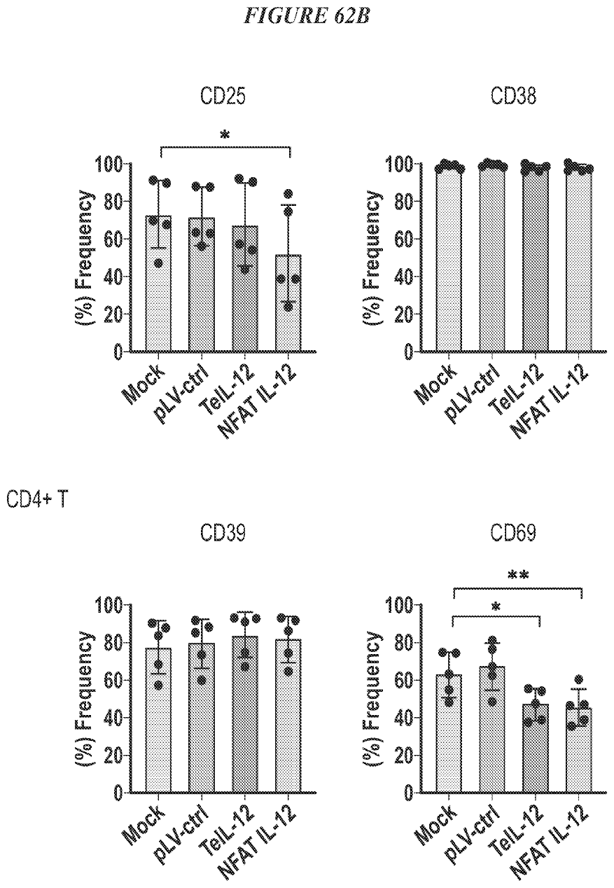

Expression of T cell activation markers CD25, CD38, CD39 and CD69 on post-REP TILs was detected by flow cytometry (FIG. 62). Increased expression of activation marker CD25 was observed in TeIL-12 post-REP TILs (CD8+ T cells), while decreased expression of CD25 was observed in NFAT-TeIL-12 post-REP TILs. No change in expression of CD38 was observed, while CD69 expression decreased.

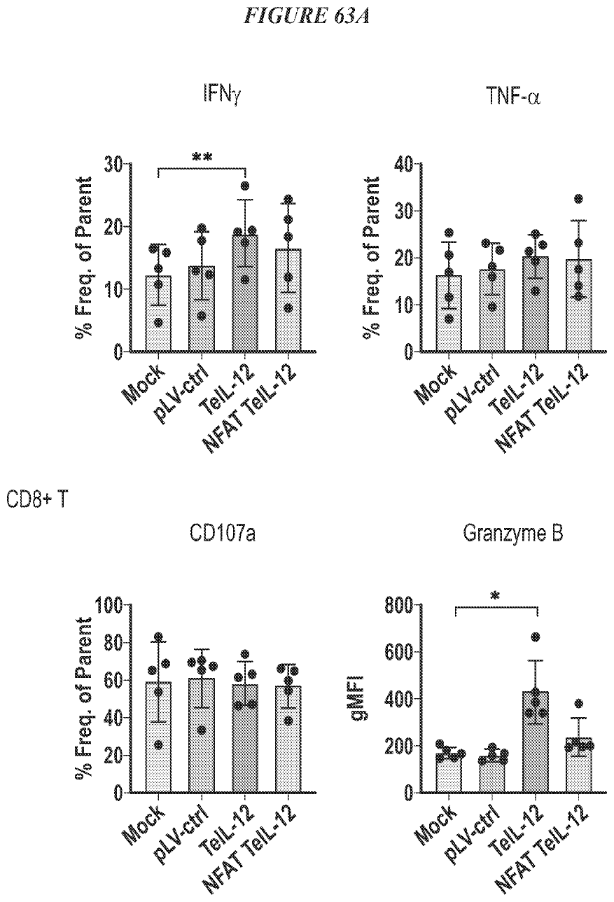

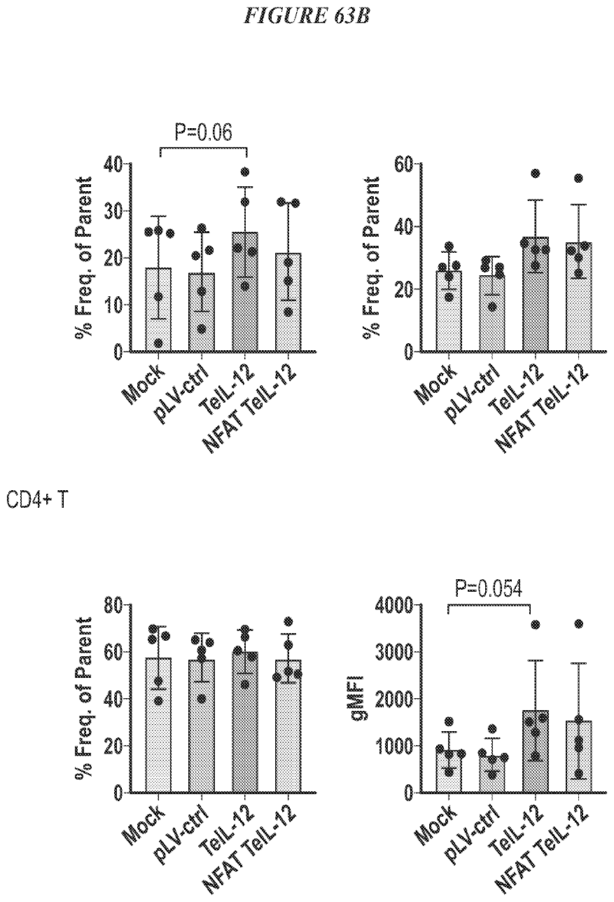

Expression of T cell function-related markers IFN-γ, TNF-α, CD107a and Granzyme B on post-REP TILs was detected by flow cytometry (FIG. 63). Increased IFN-γ and Granzyme B production was observed in TeIL-12 post-REP TILs, and more significant in CD8+ T cell population.

### Example 17: Optimization of Anti-CD3 Antibody Timing and Dosage During Rep and Impact on Expansion and Surface Expression of Tethered IL-15/IL-21

This study tested the impact on TIL expansion and surface expression of TeIL-15/TeIL21 by adding an anti-CD3 antibody at different days after the initiation of REP. The above examples are modified as described herein with regard to the timing of the anti-CD3 antibody addition; anti-CD3 antibody is added at the time as indicated in the paragraphs below.

Pre-REP TILs were prepared using the Gen 2process described in Example 7, followed by gene transduction with a lentiviral vector encoding TeIL-15 or TeIL-15 and TeIL-21 under the control of an EF-1α promotor using the protocol below.

### Viral Transduction of TILs

TILs were thawed by adding 1 mL of thawed cell suspension to 9 mL pre-warmed CM2 without gentamicin and centrifuge at 500×g, 4 min, room temperature (RT).

Supernatant was discarded and TILs were suspended in 1 mL CM2 without gentamicin+300 IU/mL IL-2 and count.

TILs were suspended at 1e6/mL CM2 without gentamicin+300 IU/mL IL-2 in a 24 well plate at 2 mL per well. Incubate overnight.

A 20× cocktail of IL-15, IL-7 was prepared for the addition of 100 uL per well.
Final concentrations: IL-15 (10 ng/mL), IL-7 (20 ng/mL) in CM2 without gentamicin
Example: 10 wells needed
   10 wells×100 uL cocktail=1 mL
      Add IL-15 to a concentration of 200 ng/mL
      Add IL-7 to a concentration of 400 ng/mL
Note: Remove 120 uL from each well before adding TransACT, IL-15, and IL-7 so that the final volume is 2 mL

100 uL of 20×cytokine cocktail was added to each TIL culture within the 24 well plate. Then, 20 uL TransACT was added to each well. Each well was pipette mixed gently to evenly disperse TILs and to mix with stimulation cocktail. Incubate for 2 days.

A Non-tissue culture 48 well plate with Retronectin (diluted 1:100 in PBS) was coated by adding 250 uL per well. Wrap plate in parafilm and then incubate at 4° C. overnight.

The coating solution solution was removed from retronectin plate and replace with 250 uL 2% BSA Fraction V. Note: The 2% BSA Fraction V was added immediately so that the well did not dry out.

Incubated at RT for 30 min for blocking.

Blocking solution was removed. The calculated volumes of viral supernatant were added and wrapped in parafilm. Centrifuged for 2000×g, 90 min, 32° C. Note: Centrifuge was warmed prior to adding plate.

While the plate was spinning with virus, TILs were collected and counted.

TILs were suspended at 1e6/mL in CM2 without gentamicin.

Once the plate finished spinning, the following was added:
1. 100 uL of TIL suspension (1e5 cells)
2. 200 uL CM2 without gentamicin
3. 300 uL CM2+IL-15 (20 ng/mL)+IL-7 (40 ng/mL)+Lentibooster 1:50
4. =Total 600 uL per well

Centrifuged 2000×g, 20 min, 32° C. Placed in incubator and incubate for 3 days.

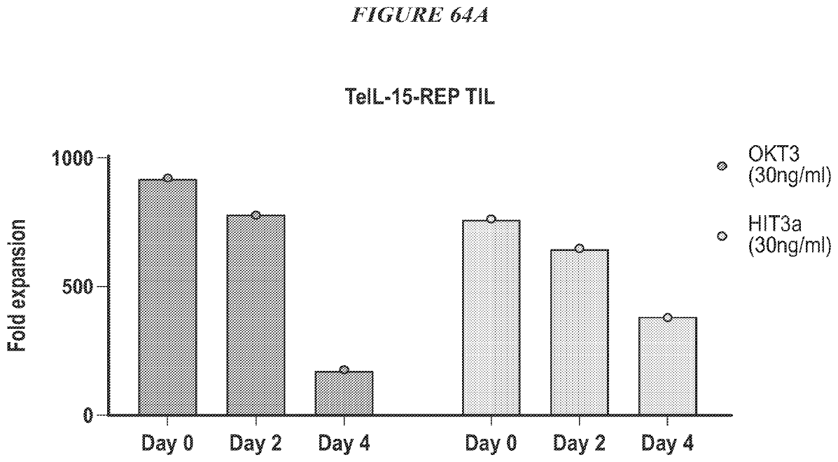

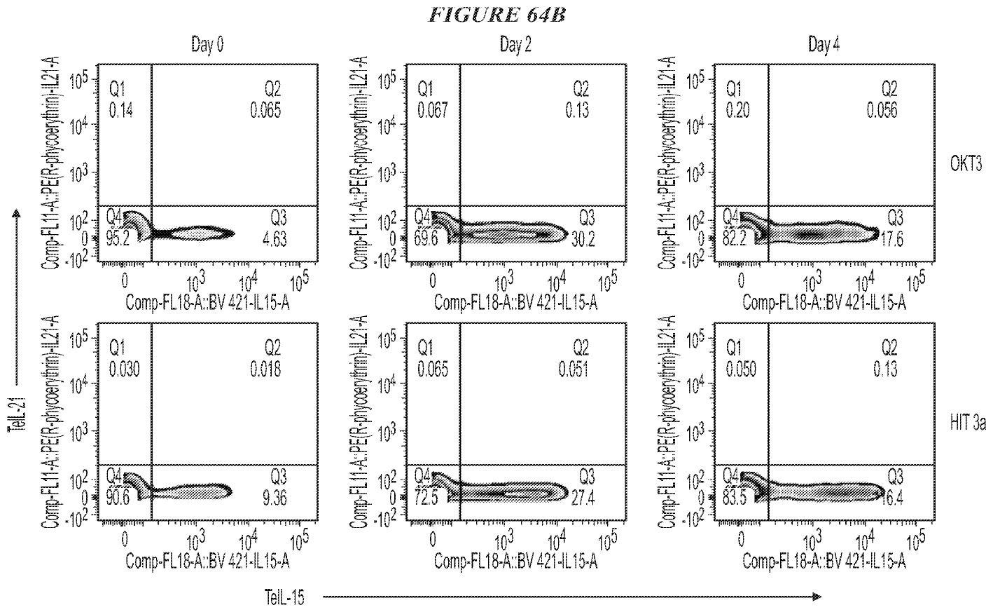

After gene transduction of a lentiviral vector expressing TeIL-15, Pre-REP TILs were processed for REP expansion with feeder cell, 3000 IU/ml IL-2 and an anti-CD3 antibody, OKT3 or HIT3a. OKT3 (30 ng/ml) or HIT3a (30 ng/ml) was added into the REP culture medium on different days (Day 0, Day 2, and Day 4) after starting the REP process. After 11 days of REP expansion, post-REP TILs were harvested. TIL expansion, and surface expression of TeIL-15/TeIL-21 were analyzed using the protocol below. Adding OKT3 or HIT3a on Day 2 or Day 4 led to increased surface expression of TeIL-15 (FIGS. 64A and B).

Surface Expression Check for Tethered Cytokine (2× Dilution with 1E5)

### Maker 20 Sample

| | |
|---|---|
| 25 ul/sample X10 | 487.5 ul DPBS |
| Live/dead 1:200 | 2.5 ul |
| FC block 1:50 | 10 ul |
| 8 mins @RT | |

### 50 Staining Buffer

| 50 ul staining buffer 20 sample | | | | | 860 ul |
|---|---|---|---|---|---|
| | CD3 | BUV737 | UCHT1 | 1.5 ul | 30 ul |
| | CD45 | PerCP | HI30 | 1 ul | 20 ul |
| | IL-21 | PE | 4BG1 | 2.5 ul | 50 ul |
| | IL 15 | Biotin | BH1543 | 2 ul | 40 ul |

Ab incubate 45 mins@4C, wash twice

| 50 ul staining buffer 20 samples | | | | 992 ul |
|---|---|---|---|---|
| | Avidin-BV421 | 1:200 | 0.25 ul | 5 ul |

Ab incubate 20 mins@4C, wash twice
Add 200 ul Staining buffer, for flow cytometry run

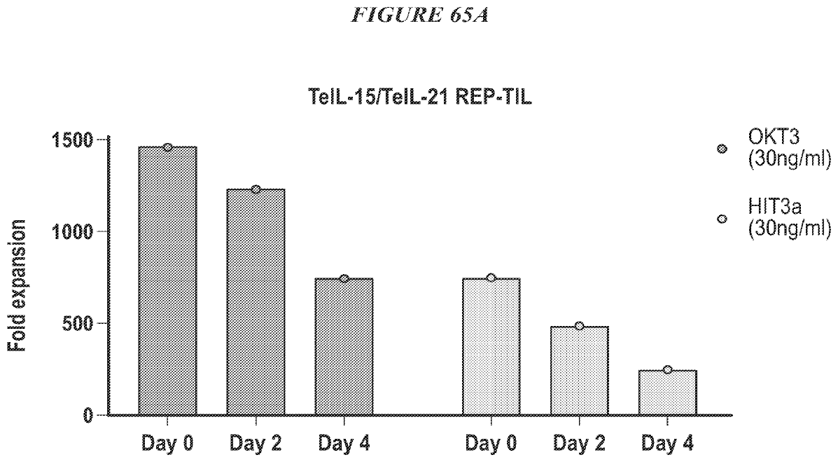

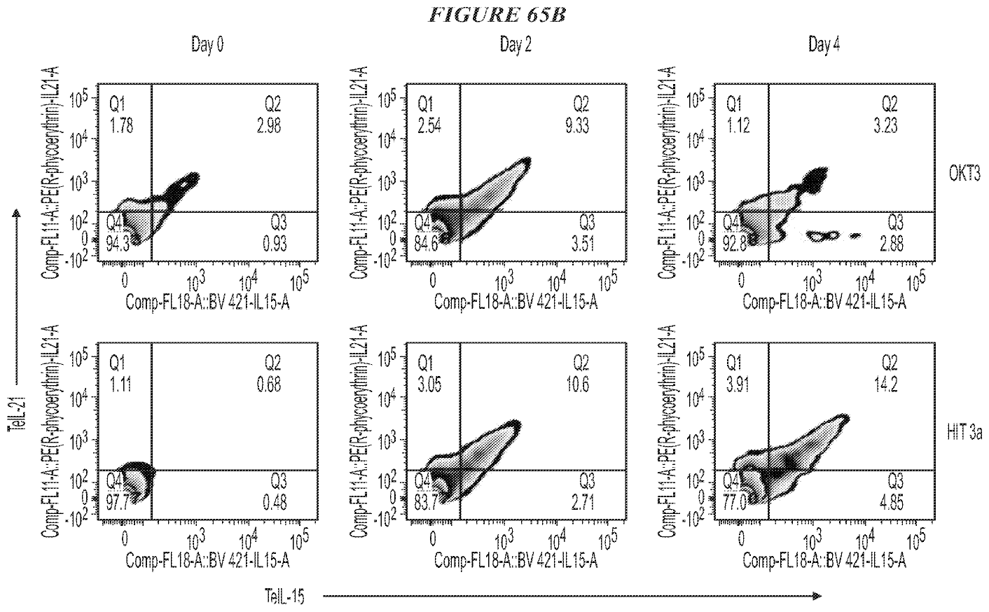

After gene transduction of a lentiviral vector expressing TeIL-15 and TeIL-21, Pre-REP TILs were processed for REP expansion with feeder cell, 3000 IU/ml IL-2 and an anti-CD3 antibody, OKT3 or HIT3a. OKT3 (30 ng/ml) or HIT3a (30 ng/ml) was added into the REP culture medium on different days (Day 0, Day 2, and Day 4) after starting the REP process. After 11 days of REP expansion, post-REP TILs were harvested. TIL expansion, and surface expression of TeIL-15/TeIL-21 were analyzed using the protocol above. Adding OKT3 or HIT3a on Day 2 or Day 4 led to increased surface expression of TeIL-15 and TeIL-21 (FIGS. 65A and 65B).

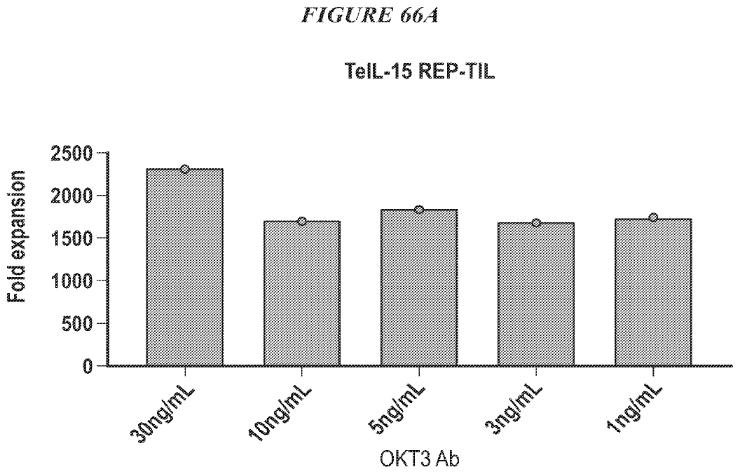

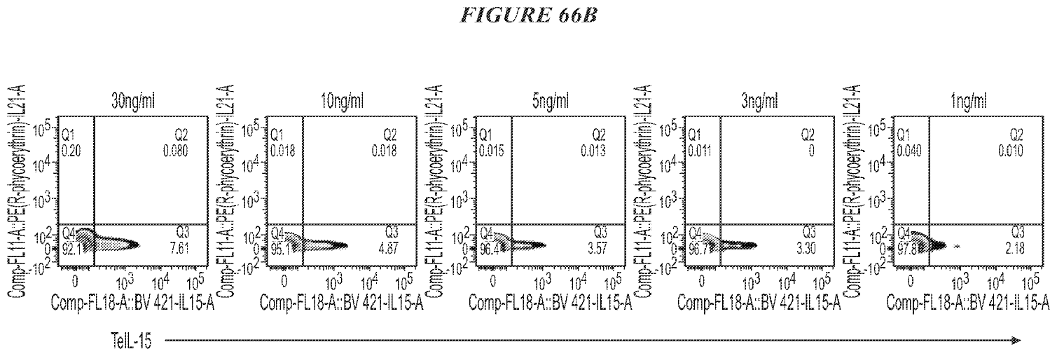

After gene transduction of a lentiviral vector expressing TeIL-15, Pre-REP TILs were processed for REP expansion with feeder cell, 3000 IU/ml IL-2 and an anti-CD3 antibody, OKT3. OKT3 was added into the REP culture medium on Day 0 at different concentrations (30 ng/mL, 10 ng/mL, 5 ng/mL, 3 ng/mL, and 1 ng/mL). After 11 days of REP expansion, post-REP TILs were harvested. TIL expansion, and surface expression of TeIL-15/TeIL-21 were analyzed using the protocol above. Adding OKT3 at 30 ng/mL led to the highest surface expression of TeIL-15 (FIGS. 66A and 66B).

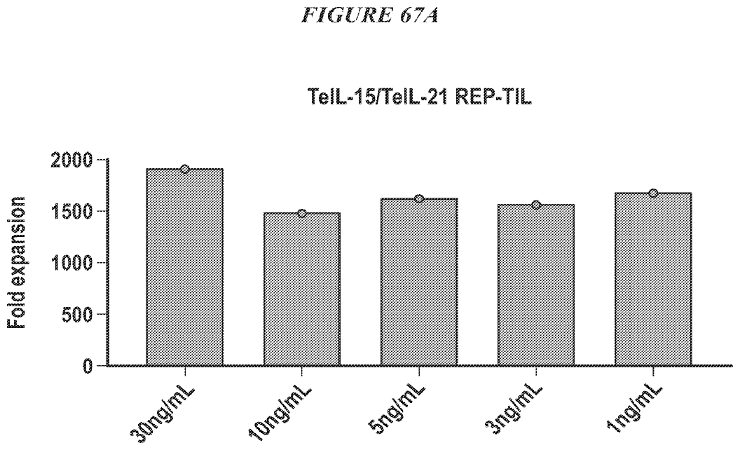

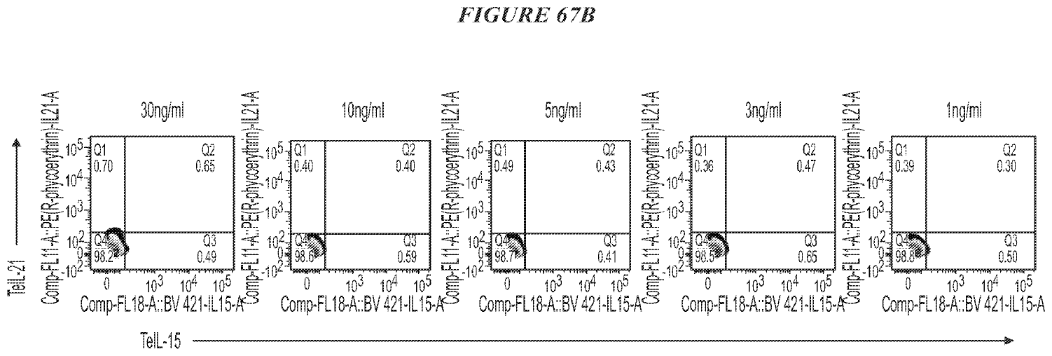

After gene transduction of a lentiviral vector expressing TeIL-15 and TeIL-21, Pre-REP TILs were processed for REP expansion with feeder cell, 3000 IU/ml IL-2 and an anti-CD3 antibody, OKT3. OKT3 was added into the REP culture medium on Day 0 at different concentrations (30 ng/mL, 10 ng/mL, 5 ng/mL, 3 ng/mL, and 1 ng/mL). After 11 days of REP expansion, post-REP TILs were harvested. TIL expansion, and surface expression of TeIL-15/TeIL-21 were analyzed using the protocol above. Adding OKT3 at all concentrations led to low surface expression of TeIL-15 and TeIL-21 (FIGS. 67A and 67B).

The examples set forth above are provided to give those of ordinary skill in the art a complete disclosure and description of how to make and use the embodiments of the compositions, systems and methods of the invention, and are not intended to limit the scope of what the inventors regard as their invention. Modifications of the above-described modes for carrying out the invention that are obvious to persons of skill in the art are intended to be within the scope of the following claims. All patents and publications mentioned in the specification are indicative of the levels of skill of those skilled in the art to which the invention pertains.

All headings and section designations are used for clarity and reference purposes only and are not to be considered limiting in any way. For example, those of skill in the art will appreciate the usefulness of combining various aspects from different headings and sections as appropriate according to the spirit and scope of the invention described herein.

All references cited herein are hereby incorporated by reference herein in their entireties and for all purposes to the same extent as if each individual publication or patent or patent application was specifically and individually indicated to be incorporated by reference in its entirety for all purposes.

Many modifications and variations of this application can be made without departing from its spirit and scope, as will be apparent to those skilled in the art. The specific embodiments and examples described herein are offered by way of example only, and the application is to be limited only by the terms of the appended claims, along with the full scope of equivalents to which the claims are entitled.

What is claimed is:

1. A method of treating a cancer in a patient or subject in need thereof comprising administering a population of tumor infiltrating lymphocytes (TILs), optionally wherein the patient or subject has received at least one prior therapy,
wherein a portion of the population of TILs are modified TILs each comprising an immunomodulatory composition associated with its surface membrane.

2. A method of treating a cancer in a patient or subject in need thereof comprising administering a population of modified tumor infiltrating lymphocytes (TILs), the method comprising the steps of:
(a) obtaining and/or receiving a first population of TILs from a tumor resected from the subject or patient by processing a tumor sample obtained from the subject into multiple tumor fragments;
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting therapeutic population of TILs obtained from step (d), wherein the transition from step (d) to step (e) occurs without opening the system;
(f) transferring the harvested TIL population from step (e) to an infusion bag, wherein the transfer from step (e) to (f) occurs without opening the system;
(g) cryopreserving the infusion bag comprising the harvested TIL population from step (f) using a cryopreservation process;
(h) administering a therapeutically effective dosage of the third population of TILs from the infusion bag in step (g) to the subject; and
(i) modifying a portion of the first, second, or third population of TILs at any time prior to the administering step (h) to generate modified TILs each comprising an immunomodulatory composition associated with its surface membrane.

3. A method of treating a cancer in a patient or subject in need thereof comprising administering a population of tumor infiltrating lymphocytes (TILs), the method comprising the steps of:
(a) obtaining a first population of TILs from a tumor resected from a subject by processing a tumor sample obtained from the subject into multiple tumor fragments;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system;
(g) cryopreserving the infusion bag comprising the harvested TIL population from step (f) using a cryopreservation process;
(h) administering a therapeutically effective dosage of the third population of TILs from the infusion bag in step (g) to the subject; and
(i) modifying a portion of the first, second, or third population of TILs at any time prior to the administering step (h) to generate modified TILs each comprising an immunomodulatory composition associated with its surface membrane.

4. A method of treating a cancer in a patient or subject in need thereof comprising administering a population of tumor infiltrating lymphocytes (TILs), the method comprising the steps of:
(a) obtaining and/or receiving a first population of TILs from surgical resection, needle biopsy, core biopsy, small biopsy, or other means for obtaining a sample that contains a mixture of tumor and TIL cells from the cancer in the patient or subject,
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system;
(g) cryopreserving the infusion bag comprising the harvested TIL population from step (f) using a cryopreservation process;
(h) administering a therapeutically effective dosage of the third population of TILs from the infusion bag in step (g) to the subject; and
(i) modifying a portion of the first, second, or third population of TILs at any time prior to the administering step (h) to generate modified TILs each comprising an immunomodulatory composition associated with its surface membrane.

5. A method of treating a cancer in a patient or subject in need thereof comprising administering a population of modified tumor infiltrating lymphocytes (TILs), the method comprising the steps of:
(a) resecting a tumor from the subject or patient, the tumor comprising a first population of TILs, optionally from surgical resection, needle biopsy, core biopsy, small biopsy, or other means for obtaining a sample that contains a mixture of tumor and TIL cells from the cancer;
(b) processing the tumor into multiple tumor fragments and adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system;
(g) cryopreserving the infusion bag comprising the harvested TIL population from step (f) using a cryopreservation process;
(h) administering a therapeutically effective dosage of the third population of TILs from the infusion bag in step (g) to the subject or patient with the cancer; and
(i) modifying a portion of the first, second, or third population of TILs at any time prior to the administering step (h) to generate modified TILs each comprising an immunomodulatory composition associated with its surface membrane.

6. A method of treating a cancer in a patient or subject in need thereof comprising administering a population of tumor infiltrating lymphocytes (TILs), the method comprising the steps of:
(a) obtaining and/or receiving a first population of TILs from surgical resection, needle biopsy, core biopsy, small biopsy, or other means for obtaining a sample that contains a mixture of tumor and TIL cells from the subject or patient;
(c) contacting the first population of TILs with a first cell culture medium;
(d) performing an initial expansion (or priming first expansion) of the first population of TILs in the first cell culture medium to obtain a second population of TILs, wherein the first cell culture medium comprises IL-2, optionally OKT-3 (anti-CD3 antibody), and optionally antigen presenting cells (APCs), where the priming first expansion occurs for a period of 1 to 8 days;
(e) performing a rapid second expansion of the second population of TILs in a second cell culture medium to obtain a third population of TILs; wherein the second cell culture medium comprises IL-2, OKT-3 (anti-CD3 antibody), and APCs; and wherein the rapid expansion is performed over a period of 14 days or less, optionally the rapid second expansion can proceed for 1 day, 2 days, 3 days, 4, days, 5 days, 6 days, 7 days, 8 days, 9 days or 10 days after initiation of the rapid second expansion;
(f) harvesting the third population of TILs;
(g) administering a therapeutically effective portion of the third population of TILs to the subject or patient with the cancer; and
(h) modifying a portion of the first, second, or third population of TILs at any time prior to the administering step (g) to generate modified TILs each comprising an immunomodulatory composition associated with its surface membrane.

7. A method of treating a cancer in a patient or subject in need thereof comprising administering a population of tumor infiltrating lymphocytes (TILs), the method comprising the steps of:
(a) resecting a tumor from the cancer in the subject or patient, the tumor comprising a first population of TILs, optionally from surgical resection, needle biopsy, core biopsy, small biopsy, or other means for obtaining a sample that contains a mixture of tumor and TIL cells from the cancer;
(b) fragmenting the tumor into tumor fragments;
(c) contacting the tumor fragments with a first cell culture medium;
(d) performing an initial expansion (or priming first expansion) of the first population of TILs in the first cell culture medium to obtain a second population of TILs, wherein the first cell culture medium comprises IL-2, optionally OKT-3 (anti-CD3 antibody), and optionally antigen presenting cells (APCs), where the priming first expansion occurs for a period of 1 to 8 days;
(e) performing a rapid second expansion of the second population of TILs in a second cell culture medium to obtain a third population of TILs; wherein the second cell culture medium comprises IL-2, OKT-3 (anti-CD3 antibody), and APCs; and wherein the rapid expansion is performed over a period of 14 days or less, optionally the rapid second expansion can proceed for 1 day, 2 days, 3 days, 4, days, 5 days, 6 days, 7 days, 8 days, 9 days or 10 days after initiation of the rapid second expansion;
(f) harvesting the third population of TILs;
(g) administering a therapeutically effective portion of the third population of TILs to the subject or patient with the cancer; and
(h) modifying a portion of the first, second, or third population of TILs at any time prior to the administering step (g) to generate modified TILs each comprising an immunomodulatory composition associated with its surface membrane.

8. A method for expanding tumor infiltrating lymphocytes (TILs) into a therapeutic population of TILs comprising:
(a) obtaining and/or receiving a first population of TILs from a tumor resected from a cancer in a subject by processing a tumor sample obtained from the tumor into multiple tumor fragments;
(b) selecting PD-1 positive TILs from the first population of TILs in step (a) to obtain a PD-1 enriched TIL population;
(c) performing a priming first expansion by culturing the PD-1 enriched TIL population in a first cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 1 to 7/8 days to obtain the second population of TILs, wherein the second population of TILs is greater in number than the first population of TILs;
(d) performing a rapid second expansion by culturing the second population of TILs in a second culture medium comprising IL-2, OKT-3, and APCs, to produce a therapeutic population of TILs, wherein the number of APCs added in the rapid second expansion is at least twice the number of APCs added in step (b), wherein the rapid second expansion is performed for a second period of about 1 to 11 days to obtain the therapeutic population of TILs, wherein the third population of TILs is a therapeutic population of TILs, wherein the rapid second expansion is performed in a container comprising a second gas-permeable surface area;
(e) harvesting the therapeutic population of TILs obtained from step (d);
(f) transferring the harvested TIL population from step (e) to an infusion bag, and
(g) modifying a portion of the first, second, or third population of TILs at any time during the method to generate modified TILs each comprising an immunomodulatory composition associated with its surface membrane.

9. A method of expanding tumor infiltrating lymphocytes (TILs) into a therapeutic population of TILs, the method comprising the steps of:
(a) obtaining and/or receiving a first population of TILs from a tumor resected from a cancer in a subject or patient by processing a tumor sample obtained from the tumor into multiple tumor fragments;
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-14 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-14 days to obtain the third population of TILs, wherein the third population of TILs is a therapeutic population of TILs, wherein the second expansion is performed in a closed container providing a second gas-permeable surface area, and wherein the transition from step (c) to step (d) occurs without opening the system;
(e) harvesting therapeutic population of TILs obtained from step (d), wherein the transition from step (d) to step (e) occurs without opening the system;
(f) transferring the harvested TIL population from step (e) to an infusion bag, wherein the transfer from step (e) to (f) occurs without opening the system; and
(g) modifying a portion of the first, second or third population of TILs at any time prior to the transfer to the infusion bag in step (f) to generate modified TILs each comprising an immunomodulatory composition associated with its surface membrane.

10. A method of expanding tumor infiltrating lymphocytes (TILs) into a therapeutic population of TILs, the method comprising the steps of:
(a) obtaining a first population of TILs from a tumor resected from a cancer in a subject by processing a tumor sample obtained from the tumor into multiple tumor fragments;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system; and
(g) modifying a portion of the first, second, or third population of TILs at any time prior to the transfer to the infusion bag in step (f) to generate modified TILs each comprising an immunomodulatory composition associated with its surface membrane.

11. A method of expanding tumor infiltrating lymphocytes (TILs) into a therapeutic population of TILs, the method comprising the steps of:
(a) obtaining and/or receiving a first population of TILs from surgical resection, needle biopsy, core biopsy, small biopsy, or other means for obtaining a sample that contains a mixture of tumor and TIL cells from a cancer in a patient or subject,
(b) adding the first population of TILs into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system; and
(g) modifying a portion of the first, second, or third population of TILs at any time prior to the transfer to the infusion bag in step (f) to generate modified TILs each comprising an immunomodulatory composition associated with its surface membrane.

12. A method of expanding tumor infiltrating lymphocytes (TILs) to a therapeutic population of TILs, the method comprising the steps of:
(a) resecting a tumor from a cancer in a subject or patient, the tumor comprising a first population of TILs, optionally from surgical resection, needle biopsy, core biopsy, small biopsy, or other means for obtaining a sample that contains a mixture of tumor and TIL cells from the cancer;
(b) adding the tumor fragments into a closed system;
(c) performing a first expansion by culturing the first population of TILs in a cell culture medium comprising IL-2 to produce a second population of TILs, wherein the first expansion is performed in a closed container providing a first gas-permeable surface area, wherein the first expansion is performed for about 3-11 days to obtain the second population of TILs, and wherein the transition from step (b) to step (c) occurs without opening the system;
(d) performing a second expansion by supplementing the cell culture medium of the second population of TILs with additional IL-2, OKT-3, and antigen presenting cells (APCs), to produce a third population of TILs, wherein the second expansion is performed for about 7-11 days to obtain the third population of TILs, wherein the second expansion is performed in a closed container providing a second gas-permeable surface area, and wherein the transition from step (c) to step (d) occurs without opening the system;
(e) harvesting the third population of TILs obtained from step (d), wherein the transition from step (d) to step (e) occurs without opening the system;
(f) transferring the harvested third TIL population from step (e) to an infusion bag, wherein the transfer from step (e) to (f) occurs without opening the system; and
(g) modifying a portion of the first, second, or third population of TILs at any time prior to the transfer to infusion bag in step (f) to generate modified TILs each comprising an immunomodulatory composition associated with its surface membrane.

13. The method of any of claims 9-12, wherein the first expansion is divided into a first step and a second step, wherein the method further comprises performing the first step of the first expansion by culturing the first population of TILs in a cell culture medium containing IL-2 to produce TILs that egress from the tumor fragments or sample, separating TILs that remain in the tumor fragments or sample from TILs that egressed from the tumor fragments or sample, optionally digesting the tumor fragments or sample to produce a tumor digest, and performing the second step of the first expansion by culturing in the cell culture medium of the TILs remaining in the tumor fragments or sample or tumor digest to produce the second population of TILs.

14. A method of expanding tumor infiltrating lymphocytes (TILs) into a therapeutic population of TILs, the method comprising the steps of:
(a) obtaining and/or receiving a first population of TILs from surgical resection, needle biopsy, core biopsy, small biopsy, or other means for obtaining a sample that contains a mixture of tumor and TIL cells from a cancer in the subject or patient;
(b) contacting the first population of TILs with a first cell culture medium;
(c) performing an initial expansion (or priming first expansion) of the first population of TILs in the first cell culture medium to obtain a second population of TILs, wherein the first cell culture medium comprises IL-2, optionally OKT-3 (anti-CD3 antibody), and optionally antigen presenting cells (APCs), where the priming first expansion occurs for a period of 1 to 8 days;
(d) performing a rapid second expansion of the second population of TILs in a second cell culture medium to obtain a third population of TILs; wherein the second cell culture medium comprises IL-2, OKT-3 (anti-CD3 antibody), and APCs; and wherein the rapid expansion is performed over a period of 14 days or less, optionally the rapid second expansion can proceed for 1 day, 2 days, 3 days, 4, days, 5 days, 6 days, 7 days, 8 days, 9 days or 10 days after initiation of the rapid second expansion;
(e) harvesting the third population of TILs; and
(f) modifying a portion of the first, second, or third population of TILs at any time prior to or after the harvesting in step (e) to generate modified TILs each comprising an immunomodulatory composition associated with its surface membrane.

15. A method of expanding tumor infiltrating lymphocytes (TILs) into a therapeutic population of TILs, the method comprising the steps of:
(a) resecting a tumor from a cancer in a subject or patient, the tumor comprising a first population of TILs, optionally from surgical resection, needle biopsy, core biopsy, small biopsy, or other means for obtaining a sample of the tumor that contains a mixture of tumor and TIL cells;
(b) fragmenting the tumor into tumor fragments;
(c) contacting the tumor fragments with a first cell culture medium;
(d) performing an initial expansion (or priming first expansion) of the first population of TILs in the first cell culture medium to obtain a second population of TILs, wherein the first cell culture medium comprises IL-2, optionally OKT-3 (anti-CD3 antibody), and optionally antigen presenting cells (APCs), where the priming first expansion occurs for a period of 1 to 8 days;
(e) performing a rapid second expansion of the second population of TILs in a second cell culture medium to obtain a third population of TILs; wherein the second cell culture medium comprises IL-2, OKT-3 (anti-CD3 antibody), and APCs; and wherein the rapid expansion is performed over a period of 14 days or less, optionally the rapid second expansion can proceed for 1 day, 2 days, 3 days, 4, days, 5 days, 6 days, 7 days, 8 days, 9 days or 10 days after initiation of the rapid second expansion;
(f) harvesting the third population of TILs; and
(g) modifying a portion of the first, second, or third population of TILs at any time prior to or after the harvesting in step (f) to generate modified TILs each comprising an immunomodulatory composition associated with its surface membrane.

16. A method for expanding tumor infiltrating lymphocytes (TILs) into a therapeutic population of TILs comprising:
(a) obtaining and/or receiving a first population of TILs from a tumor resected from a cancer in a subject by processing a tumor sample obtained from the tumor into multiple tumor fragments;
(b) performing a priming first expansion by culturing the first population of TILs in a cell culture medium comprising IL-2, optionally OKT-3, and optionally comprising antigen presenting cells (APCs), to produce a second population of TILs, wherein the priming first expansion is performed for a first period of about 1 to 7/8 days to obtain the second population of TILs, wherein the second population of TILs is greater in number than the first population of TILs;
(c) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs;
(d) harvesting the therapeutic population of TILs obtained from step (c); and
(e) modifying a portion of the first, second, or third population of TILs at any time prior to or after the harvesting in step (d) to generate modified TILs each comprising an immunomodulatory composition associated with its surface membrane.

17. The method of claim 16, wherein in step (b) the cell culture medium further comprises antigen-presenting cells (APCs), and wherein the number of APCs in the culture medium in step (c) is greater than the number of APCs in the culture medium in step (b).

18. A method for expanding tumor infiltrating lymphocytes (TILs) into a therapeutic population of TILs comprising:
(a) performing a priming first expansion by culturing a first population of TILs, said first population of TILs obtainable by processing a tumor sample from a tumor resected from a cancer in a subject into multiple tumor fragments, in a cell culture medium comprising IL-2, optionally OKT-3, and optionally antigen presenting cells (APCs) to produce a second population of TILs, wherein the priming first expansion is performed in a container comprising a first gas-permeable surface area, wherein the priming first expansion is performed for first period of about 1 to 7/8 days to obtain the second population of TILs, wherein the second population of TILs is greater in number than the first population of TILs;
(b) performing a rapid second expansion by contacting the second population of TILs to a cell culture medium of the second population of TILs with additional IL-2, OKT-3, and APCs, to produce a third population of TILs, wherein the number of APCs in the rapid second expansion is at least twice the number of APCs in step (a), wherein the rapid second expansion is performed for a second period of about 1 to 11 days to obtain the third population of TILs, wherein the third population of TILs is a therapeutic population of TILs, wherein the rapid second expansion is performed in a container comprising a second gas-permeable surface area;
(c) harvesting the therapeutic population of TILs obtained from step (b); and
(d) modifying a portion of the first, second, or third population of TILs at any time prior to or after the harvesting in step (c) to generate modified TILs each comprising an immunomodulatory composition associated with its surface membrane.

19. A method for expanding tumor infiltrating lymphocytes (TILs) into a therapeutic population of TILs comprising:
(a) performing a priming first expansion by culturing a first population of TILs in a cell culture medium comprising IL-2, optionally OKT-3, and optionally comprising antigen presenting cells (APCs), to produce a second population of TILs, wherein the priming first expansion is performed for a first period of about 1 to 7/8 days to obtain the second population of TILs, wherein the second population of TILs is greater in number than the first population of TILs;
(b) performing a rapid second expansion by contacting the second population of TILs with a cell culture medium comprising IL-2, OKT-3, and APCs, to produce a third population of TILs, wherein the rapid second expansion is performed for a second period of about 1 to 11 days to obtain the third population of TILs, wherein the third population of TILs is a therapeutic population of TILs;
(c) harvesting the therapeutic population of TILs obtained from step (b); and
(d) modifying a portion of the first, second, or third population of TILs at any time prior to or after the harvesting in step (c) to generate modified TILs each comprising an immunomodulatory composition associated with its surface membrane.

20. The method of claim 19, wherein in step (a) the cell culture medium further comprises antigen-presenting cells (APCs), and wherein the number of APCs in the culture medium in step (c) is greater than the number of APCs in the culture medium in step (b).

21. The method of any of claims 14-18, wherein the priming first expansion is divided into a first step and a second step, wherein the method further comprises performing the first step of the priming first expansion by culturing the first population of TILs in a cell culture medium containing IL-2 to produce TILs that egress from the tumor fragments or sample, separating TILs that remain in the tumor fragments or sample from TILs that egressed from the tumor fragments or sample, optionally digesting the tumor fragments or sample to produce a tumor digest, and performing the second step of the priming first expansion by culturing in the cell culture medium the TILs remaining in the tumor fragments or sample or tumor digest to produce the second population of TILs.

22. A method for expanding tumor infiltrating lymphocytes (TILs) into a therapeutic population of TILs comprising:
(a) obtaining and/or receiving a first population of TILs from a tumor sample obtained from one or more small biopsies, core biopsies, or needle biopsies of a tumor from a cancer in a subject by culturing the tumor sample in a first cell culture medium comprising IL-2 for about 3 days;
(b) performing a priming first expansion by culturing the first population of TILs in a second cell culture medium comprising IL-2, OKT-3, and antigen presenting cells (APCs) to produce a second population of TILs, wherein the priming first expansion is performed in a container comprising a first gas-permeable surface area, wherein the priming first expansion is performed for first period of about 7 or 8 days to obtain the second population of TILs, wherein the second population of TILs is greater in number than the first population of TILs;
(c) performing a rapid second expansion by supplementing the second cell culture medium of the second population of TILs with additional IL-2, OKT-3, and APCs, to produce a third population of TILs, wherein the number of APCs added in the rapid second expansion is at least twice the number of APCs added in step (b), wherein the rapid second expansion is performed for a second period of about 11 days to obtain the third population of TILs, wherein the third population of TILs is a therapeutic population of TILs, wherein the rapid second expansion is performed in a container comprising a second gas-permeable surface area;
(d) harvesting the therapeutic population of TILs obtained from step (c);
(e) transferring the harvested TIL population from step (d) to an infusion bag; and
(f) modifying a portion of the first, second, or third population of TILs at any time prior to transfer to the infusion bag in step (e) to generate modified TILs each comprising an immunomodulatory composition associated with its surface membrane.

23. A method for expanding tumor infiltrating lymphocytes (TILs) into a therapeutic population of TILs comprising:
(a) obtaining and/or receiving a first population of TILs from a tumor sample obtained from one or more small biopsies, core biopsies, or needle biopsies of a tumor from a cancer in a subject by culturing the tumor sample in a first cell culture medium comprising IL-2 for about 3 days;
(b) performing a priming first expansion by culturing the first population of TILs in a second cell culture medium comprising IL-2, OKT-3, and antigen presenting cells (APCs) to produce a second population of TILs, wherein the priming first expansion is performed for first period of about 7 or 8 days to obtain the second population of TILs, wherein the second population of TILs is greater in number than the first population of TILs;
(c) performing a rapid second expansion by contacting the second population of TILs with a third cell culture medium comprising IL-2, OKT-3, and APCs, to produce a third population of TILs, wherein the rapid second expansion is performed for a second period of about 11 days to obtain the third population of TILs, wherein the third population of TILs is a therapeutic population of TILs;
(d) harvesting the therapeutic population of TILs obtained from step (c); and
(e) modifying a portion of the first, second, or third population of TILs at any time prior to or after the harvesting in step (d) to generate modified TILs each comprising an immunomodulatory composition associated with its surface membrane.

24. The method of any one of claims 1-18 and 21-23, wherein the cancer is selected from the group consisting of melanoma, ovarian cancer, cervical cancer, non-small-cell lung cancer (NSCLC), lung cancer, bladder cancer, breast cancer, triple negative breast cancer, cancer caused by human papilloma virus, head and neck cancer (including head and neck squamous cell carcinoma (HNSCC)), renal cancer, and renal cell carcinoma.

25. A method of expanding T cells comprising:
(a) performing a priming first expansion of a first population of T cells obtained from a donor by culturing the first population of T cells to effect growth and to prime an activation of the first population of T cells;
(b) after the activation of the first population of T cells primed in step (a) begins to decay, performing a rapid second expansion of the first population of T cells by culturing the first population of T cells to effect growth and to boost the activation of the first population of T cells to obtain a second population of T cells;
(c) harvesting the second population of T cells; and
(d) modifying a portion of the first or second population of T cells at any time prior to or after the harvesting in step (c) to generate modified T cells each comprising an immunomodulatory composition associated with its surface membrane.

26. A method of expanding T cells comprising:
(a) performing a priming first expansion of a first population of T cells from a tumor sample obtained from one or more small biopsies, core biopsies, or needle biopsies of a tumor in a donor by culturing the first population of T cells to effect growth and to prime an activation of the first population of T cells;
(b) after the activation of the first population of T cells primed in step (a) begins to decay, performing a rapid second expansion of the first population of T cells by culturing the first population of T cells to effect growth and to boost the activation of the first population of T cells to obtain a second population of T cells;
(c) harvesting the second population of T cells; and
(d) modifying a portion of the first or second population of T cells at any time prior to or after the harvesting in step (e) to generate modified T cells each comprising an immunomodulatory composition associated with its surface membrane.

27. A method for expanding peripheral blood lymphocytes (PBLs) from peripheral blood, the method comprising the steps of:
(a) obtaining a sample of peripheral blood mononuclear cells (PBMCs) from peripheral blood of a patient;
(b) culturing said PBMCs in a culture comprising a first cell culture medium with IL-2, anti-CD3/anti-CD28 antibodies and a first combination of antibiotics, for a period of time selected from the group consisting of: about 9 days, about 10 days, about 11 days, about 12 days, about 13 days and about 14 days, thereby effecting expansion of peripheral blood lymphocytes (PBLs) from said PBMCs;
(c) harvesting the PBLs from the culture in step (b); and
(d) modifying a portion of the PBLs at any time prior to or after the harvesting in step (c) to generate modified PBLs each comprising an immunomodulatory composition associated with its surface membrane.

28. The method of claim 27, wherein the patient is pre-treated with ibrutinib or another interleukin-2 inducible T cell kinase (ITK) inhibitor.

29. The method of claim 27 or 28, wherein the patient is refractory to treatment with ibrutinib or another ITK inhibitor.

30. The method of any one of claims 1-29, wherein the immunomodulatory composition comprises one or more membrane anchored immunomodulatory fusion proteins each comprising one or more immunomodulatory agents and a cell membrane anchor moiety.

31. The method of claim 30, wherein the one or more immunomodulatory agents comprise one or more cytokines.

32. The method of claim 31, wherein the one or more cytokines comprise one or more of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof.

33. The method of claim 32, wherein the one or more cytokines comprise IL-12 or a variant thereof.

34. The method of claim 33, wherein the IL-12 comprises a human IL-12 p35 subunit attached to a human IL-12 p40 subunit.

35. The method of claim 34, wherein the human IL-12 p35 subunit has the amino acid sequence of SEQ ID NO:247 and the human IL-12 p40 subunit has the amino acid sequence of SEQ ID NO:248.

36. The method of claim 32, wherein the one or more cytokines comprise IL-15 or a variant thereof.

37. The method of claim 36, wherein the IL-15 is human IL-15.

38. The method of claim 37, wherein the human IL-15 has the amino acid sequence of SEQ ID NO:258.

39. The method of claim 32, wherein the one or more cytokines comprise IL-18 or a variant thereof.

40. The method of claim 39, wherein the IL-18 is human IL-18.

41. The method of claim 40, wherein the human IL-18 has the amino acid sequence of any one of SEQ ID NOs:269, 270, and 331-385.

42. The method of claim 32, wherein the one or more cytokines comprise IL-21 or a variant thereof.

43. The method of claim 42, wherein the IL-21 is human IL-21.

44. The method of claim 43, wherein the human IL-21 has the amino acid sequence of SEQ ID NO:271.

45. The method of claim 30, wherein the one or more immunomodulatory agents comprise a CD40 agonist.

46. The method of claim 45, wherein the CD40 agonist is an anti-CD40 binding domain or CD40L.

47. The method of claim 46, wherein the CD40 agonist is a CD40 binding domain comprising a variable heavy domain (VH) and a variable light domain (VL).

48. The method of claim 47, wherein the VH and VL of the CD40 binding domain are selected from the following:
a. a VH having the amino acid sequence of SEQ ID NO: 274, and a VL having the amino acid sequence of SEQ ID NO:275;
b. a VH having the amino acid sequence of SEQ ID NO: 277, and a VL having the amino acid sequence of SEQ ID NO:278;
c. a VH having the amino acid sequence of SEQ ID NO: 280, and a VL having the amino acid sequence of SEQ ID NO:281; and
d. a VH having the amino acid sequence of SEQ ID NO: 283, and a VL having the amino acid sequence of SEQ ID NO:284.

49. The method of claim 47 or 48, wherein the CD40 binding domain is an scFv.

50. The method of claim 46, wherein the CD40 agonist is a human CD40L having the amino acid sequence of SEQ ID NO: 273.

51. The method of any one of claims 30 to 50, wherein the membrane anchored immunomodulatory fusion protein is according to the formula, from N- to C-terminus: S-IA-L-C, wherein S is a signal peptide, IA is an immunomodulatory agent, L is a linker and C is a cell membrane anchor moiety.

52. The method of claim 51, wherein the cell membrane anchor moiety comprises a CD8a transmembrane-intracellular domain, a B7-1 transmembrane domain, a B7-2 transmembrane domain, or a CD8a transmembrane domain.

53. The method of claim 52, wherein the cell membrane anchor moiety comprises a B7-1 transmembrane domain.

54. The method of claim 53, wherein the cell membrane anchor moiety has the amino acid sequence of SEQ ID NO:239.

55. The method of any one of claims 30 to 54, wherein the immunomodulatory composition comprises two or more different membrane anchored immunomodulatory fusion proteins, wherein each of the different membrane anchored immunomodulatory fusion proteins each comprises a different immunomodulatory agent.

56. The method of claim 55, wherein the different immunomodulatory agents are selected from: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof, and a CD40 agonist.

57. The method of claim 56, wherein the different immunomodulatory agents are selected from: IL-12 and IL-15, IL-15 and IL-18, CD40L and IL-15, IL-15 and IL-21, IL-2 and IL-12, and a variant thereof.

58. The method of any one of claims 30 to 57, wherein the modifying comprises introducing a heterologous nucleic acid encoding the fusion protein into the portion of TILs and expressing the fusion protein on the surface of the modified TILs.

59. The method of claim 58, wherein the heterologous nucleic acid comprises an adenoviral vector, a retroviral vector, a lentiviral vector, an adeno-associated vector (AAV), or a piggyBac transposon.

60. The method of claim 58 or 59, wherein the heterologous nucleic acid comprises an NFAT promoter, an EF-1a promotor, an MND promoter, or an SSFV promotor.

61. The method of any one of claims 1-29, wherein immunomodulatory composition comprises a fusion protein comprising one or more immunomodulatory agents linked to a TIL surface antigen binding domain.

62. The method of claim 61, wherein the one or more immunomodulatory agents comprise one or more cytokines.

63. The method of claim 62, wherein the one or more cytokines comprises one or more of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof.

64. The method of claim 63, wherein the one or more cytokines comprise IL-12 or a variant thereof.

65. The method of claim 63, wherein the one or more cytokines comprise IL-15 or a variant thereof.

66. The method of claim 63, wherein the one or more cytokines comprises IL-21 or a variant thereof.

67. The method of claim 63, wherein the one or more cytokines comprise IL-18 or a variant thereof.

68. The method of claim 67, wherein the one or more cytokines comprise DR-IL-18.

69. The method of any one of claims 61 to 68, wherein the TIL surface antigen binding domain comprises an antibody variable heavy domain and variable light domain.

70. The method of any one of claims 61 to 69, wherein the TIL surface antigen binding domain comprises an antibody or fragment thereof.

71. The method of any one of claims 43 to 50, wherein the TIL surface antigen binding domain exhibits an affinity for one or more of following TIL surface antigens: CD45, CD4, CD8, CD3, CDlla, CDllb, CDllc, CD18, CD25, CD127, CD19, CD20, CD22, HLA-DR, CD197, CD38, CD27, CD196, CXCR3, CXCR4, CXCR5, CD84, CD229, CCR1, CCR5, CCR4, CCR6, CCR8, CCR10, CD 16, CD56, CD 137, OX40, or GITR.

72. The method of any one of claims 61 to 71, wherein the modifying comprises incubating the fusion protein with the portion of TILs under conditions to permit the binding of the fusion protein to the portion of TILs.

73. The method of any one of claims 1 to 29, wherein immunomodulatory composition comprises a nanoparticle comprising a plurality of immunomodulatory agents.

74. The method of claim 73, wherein the plurality of immunomodulatory agents are covalently linked together by degradable linkers.

75. The method of claim 74, wherein the nanoparticle comprises at least one polymer, cationic polymer, or cationic block co-polymer on the nanoparticle surface.

76. The method of any one of claims 73 to 75, wherein the one or more cytokines comprises one or more of IL-2, IL-6, IL-7, IL-9, IL-12, IL-15, IL-18, IL-21, IL-23, IL-27, IFNgamma, TNFa, IFN alpha, IFN beta, GM-CSF, GCSF, or a variant thereof.

77. The method of claim 76, wherein the one or more cytokines comprises IL-12 or a variant thereof.

78. The method of claim 76, wherein the one or more cytokines comprises IL-15 or a variant thereof.

79. The method of claim 76, wherein the one or more cytokines comprises IL-21 or a variant thereof.

80. The method of claim 76, wherein the one or more cytokines comprises IL-18 or a variant thereof.

81. The method of claim 80, wherein the one or more cytokines comprise DR-IL-18.

82. The method of any one of claims 73 to 81, wherein the nanoparticle is a liposome, a protein nanogel, a nucleotide nanogel, a polymer nanoparticle, or a solid nanoparticle.

83. The method of claim 82, wherein the nanoparticle is a nanogel.

84. The method of any one of claims 73 to 83, wherein the nanoparticle further comprises an antigen binding domain that binds to one or more of the following antigens: CD45, CDlla (integrin alpha-L), CD 18 (integrin beta-2), CD1lb, CD1lc, CD25, CD8, or CD4.

85. The method of any one of claims 73 to 81, wherein the modifying comprises attaching the immunomodulatory composition to the surface of the portion of TILs.

86. The method according to any of claims 2 to 5 or 9 to 13, wherein the modifying is carried out on TILs from the first expansion, or TILs from the second expansion, or both.

87. The method according to any of claims 6 to 8 or 14 to 23, wherein the modifying is carried out on TILs from the priming first expansion, or TILs from the rapid second expansion, or both.

88. The method according to any of claims 2 to 5 or 9 to 13, wherein the modifying is carried out after the first expansion and before the second expansion.

89. The method according to any of claims 6 to 8 or 14 to 23, wherein the modifying is carried out after the priming first expansion and before the rapid second expansion, or both.

90. The method according to any of claims 2 to 5 or 9 to 13, wherein the modifying is carried out after the second expansion.

91. The method according to any of claims 6 to 8 or 14 to 23, wherein the modifying is carried out after the rapid second expansion.

92. The method according to any of claims 2 to 72, wherein the modifying is carried out after the harvesting.

93. The method of any one of claims 2 to 5 or 9 to 13, wherein the first expansion is performed over a period of about 11 days.

94. The method of any one of claims 6 to 8 or 14 to 23, wherein the priming first expansion is performed over a period of about 11 days.

95. The method of any one of claims 2 to 5 or 9 to 13, wherein the IL-2 is present at an initial concentration of between 1000 IU/mL and 6000 IU/mL in the cell culture medium in the first expansion.

96. The method of any one of claims 6 to 8 or 14 to 23, wherein the IL-2 is present at an initial concentration of between 1000 IU/mL and 6000 IU/mL in the cell culture medium in the priming first expansion.

97. The method of any one of claims 2 to 5 or 9 to 13, wherein in the second expansion step, the IL-2 is present at an initial concentration of between 1000 IU/mL and 6000 IU/mL and the OKT-3 antibody is present at an initial concentration of about 30 ng/mL.

98. The method of any one of claims 6-8 or 14-23, wherein in the rapid second expansion step, the IL-2 is present at an initial concentration of between 1000 IU/mL and 6000 IU/mL and the OKT-3 antibody is present at an initial concentration of about 30 ng/mL.

99. The method of any one of claims 2 to 5 or 9 to 13, wherein the first expansion is performed using a gas permeable container.

100. The method of any one of claims 6 to 8 or 14 to 23, wherein the priming first expansion is performed using a gas permeable container.

101. The method of any one of claims 2 to 5 or 9 to 13, wherein the second expansion is performed using a gas permeable container.

102. The method of any one of claims 6 to 8 or 14 to 23, wherein the rapid second expansion is performed using a gas permeable container.

103. The method of any one of claims 2 to 5 or 9 to 13, wherein the cell culture medium of the first expansion further comprises a cytokine selected from the group consisting of IL-4, IL-7, IL-15, IL-21, and combinations thereof.

104. The method of any one of claims 6 to 8 or 14 to 23, wherein the cell culture medium of the priming first expansion further comprises a cytokine selected from the group consisting of IL-4, IL-7, IL-15, IL-21, and combinations thereof.

105. The method of any one of claims 2 to 5 or 9 to 13, wherein the cell culture medium of the second expansion further comprises a cytokine selected from the group consisting of IL-4, IL-7, IL-15, IL-21, and combinations thereof.

106. The method of any one of claims 6 to 8 or 14 to 23, wherein the cell culture medium of the rapid second expansion further comprises a cytokine selected from the group consisting of IL-4, IL-7, IL-15, IL-21, and combinations thereof.

107. The method of any one of claims 1 to 8, further comprising the step of treating the patient with a non-myeloablative lymphodepletion regimen prior to administering the TILs to the patient.

108. The method of claim 107, wherein the non-myeloablative lymphodepletion regimen comprises the steps of administration of cyclophosphamide at a dose of 60 mg/m²/day for two days followed by administration of fludarabine at a dose of 25 mg/m²/day for three days.

109. The method of claim 107, wherein the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for three days.

110. The method of claim 107, wherein the non-myeloablative lymphodepletion regimen comprises the steps of administration of cyclophosphamide at a dose of 60 mg/m²/day and fludarabine at a dose of 25 mg/m²/day for two days followed by administration of fludarabine at a dose of 25 mg/m²/day for one day.

111. The method of any one of claims 108 to 110, wherein the cyclophosphamide is administered with mesna.

112. The method of any one of claims 1 to 7 or 107 to 111, further comprising the step of treating the patient with an IL-2 regimen starting on the day after the administration of TILs to the patient.

113. The method of any one of claims 1 to 7 or 107 to 111, further comprising the step of treating the patient with an IL-2 regimen starting on the same day as administration of TILs to the patient.

114. The method of claim 112 or 113, wherein the IL-2 regimen is a high-dose IL-2 regimen comprising 600,000 or 720,000 IU/kg of aldesleukin, or a biosimilar or variant thereof, administered as a 15-minute bolus intravenous infusion every eight hours until tolerance.

115. The method according to any one of claims 1 to 7 or 107 to 114, wherein a therapeutically effective population of TILs is administered and comprises from about 2.3×10¹⁰ to about 13.7×10¹⁰ TILs.

116. The method of any one of 6 to 8 or 14 to 23, wherein the priming first expansion and rapid second expansion are performed over a period of 21 days or less.

117. The method of any one of claims 6 to 8 or 14 to 23, wherein the priming first expansion and rapid second expansion are performed over a period of 16 or 17 days or less.

118. The method of any one of claims 6 to 8 or 14 to 23, wherein the priming first expansion is performed over a period of 7 or 8 days or less.

119. The method of any one of claims 6 to 8 or 14 to 23, wherein the rapid second expansion is performed over a period of 11 days or less.

120. The method of any one of claims 2 to 5 or 9 to 13, the first expansion in step (c) and the second expansion in step (d) are each individually performed within a period of 11 days.

121. The method of any one of claims 2 to 5 or 9 to 13, wherein steps (a) through (f) are performed in about 10 days to about 22 days.

122. The method according to any of claims 1 to 121, wherein the modified TILs further comprise a genetic modification that causes expression of one or more immune checkpoint genes to be silenced or reduced in at least a portion of the therapeutic population of TILs.

123. The method according to claim 122, wherein said one or more immune checkpoint genes is/are selected from the group comprising PD-1, CTLA-4, LAG-3, HAVCR2 (TIM-3), Cish, TGFβ, PKA, CBL-B, PPP2CA, PPP2CB, PTPN6, PTPN22, PDCD1, BTLA, CD160, TIGIT, TET2, CD96, CRTAM, LAIR1, SIGLEC7, SIGLEC9, CD244, TNFRSF10B, TNFRSF10A, CASP8, CASP10, CASP3, CASP6, CASP7, FADD, FAS, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, GUCY1A2, GUCY1A3, GUCY1B2, GUCY1B3, TOX, SOCS1, ANKRD11, and BCOR.

124. The method according to claim 122, wherein said one or more immune checkpoint genes is/are selected from the group comprising PD-1, TIGIT, CTLA-4, LAG-3, HAVCR2 (TIM-3), Cish, TGFβ, and PKA.

125. The method according to any of claims 1 to 124, wherein the modified TILs further comprises a genetic modification that causes expression of one or more immune checkpoint genes to be enhanced in at least a portion of the therapeutic population of TILs, the immune checkpoint gene(s) being selected from the group comprising CCR2, CCR4, CCR5, CXCR2, CXCR3, CX3CR1, IL-2, IL-4, IL-7, IL-10, IL-15, IL-21, the NOTCH 1/2 intracellular domain (ICD), and/or the NOTCH ligand mDLL1.

126. The method according to any of claims 122-125, wherein the genetic modification is produced using a programmable nuclease that mediates the generation of a double-strand or single-strand break at said one or more immune checkpoint genes.

127. The method according to any of claims 122-125, wherein the genetic modification is produced using one or more methods selected from an RNA interference method (e.g., shRNA), a CRISPR method, a TALE method, a zinc finger method, a Cas-CLOVER method, and a combination thereof.

128. The method of claim 127, wherein the genetic modification is produced using a CRISPR method.

129. The method of claim 128, wherein the CRISPR method is a CRISPR/Cas9 method.

130. The method of claim 127, wherein genetic modification is produced using a TALE method.

131. The method of claim 127, wherein the genetic modification is produced using a zinc finger method.

132. The method of any of claims 1 to 23 or 86 to 121, wherein the modified TILs transiently express the immunomodulatory composition on the cell surface.

133. The method of claim 132, wherein the immunomodulatory composition comprises one or more membrane anchored immunomodulatory fusion proteins, wherein each fusion protein comprises one or more immunomodulatory agents and a cell membrane anchor moiety.

134. The method of claim 127, wherein the genetic modification is produced using an RNA interference method (e.g., shRNA).

135. The method of claim 134, wherein the one or more immunomodulatory agents comprise one or more cytokines.

136. The method of claim 135, wherein the one or more cytokines comprise one or more of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof.

137. The method of claim 136, wherein the one or more cytokines comprise IL-2 or a variant thereof.

138. The method of claim 137, wherein the IL-2 is human IL-2.

139. The method of claim 138, wherein the human IL-2 has the amino acid sequence of SEQ ID NO:272.

140. The method of claim 136, wherein the one or more cytokines comprise IL-12 or a variant thereof.

141. The method of claim 140, wherein the IL-12 comprises a human IL-12 p35 subunit attached to a human IL-12 p40 subunit.

142. The method of claim 141, wherein the human IL-12 p35 subunit has the amino acid sequence of SEQ ID NO:267 and the human IL-12 p40 subunit has the amino acid sequence of SEQ ID NO:268.

143. The method of claim 136, wherein the one or more cytokines comprise IL-15 or a variant thereof.

144. The method of claim 143, wherein the IL-15 is human IL-15.

145. The method of claim 144, wherein the human IL-15 has the amino acid sequence of SEQ ID NO:258.

146. The method of claim 136, wherein the one or more cytokines comprise IL-18 or a variant thereof.

147. The method of claim 146, wherein the IL-18 is human IL-18.

148. The method of claim 147, wherein the human IL-18 has the amino acid sequence of any one of SEQ ID NOs:269, 270, and 331-385.

149. The method of claim 136, wherein the one or more cytokines comprise IL-21 or a variant thereof.

150. The method of claim 149, wherein the IL-21 is human IL-21.

151. The method of claim 150, wherein the human IL-21 has the amino acid sequence of SEQ ID NO:271.

152. The method of claim 134, wherein the one or more immunomodulatory agents comprises a CD40 agonist.

153. The method of claim 152, wherein the CD40 agonist is an anti-CD40 binding domain or CD40L.

154. The method of claim 153, wherein the CD40 agonist is a CD40 binding domain comprising a variable heavy domain (VH) and a variable light domain (VL).

155. The method of claim 154, wherein the VH and VL of the CD40 binding domain are selected from the following:
a. a VH having the amino acid sequence of SEQ ID NO: 274, and a VL having the amino acid sequence of SEQ ID NO:275;
b. a VH having the amino acid sequence of SEQ ID NO: 277, and a VL having the amino acid sequence of SEQ ID NO:278;
c. a VH having the amino acid sequence of SEQ ID NO: 280, and a VL having the amino acid sequence of SEQ ID NO:281; and
d. a VH having the amino acid sequence of SEQ ID NO: 283, and a VL having the amino acid sequence of SEQ ID NO:284.

156. The method of claim 154 or 155, wherein the CD40 binding domain is an scFv.

157. The method of claim 152, wherein the CD40 agonist is a human CD40L having the amino acid sequence of SEQ ID NO: 273.

158. The method of any one of claims 134 to 157, wherein the one or more membrane anchored immunomodulatory fusion proteins are independently according to the formula, from N- to C-terminus: S-IA-L-C, wherein S is a signal peptide, IA is an immunomodulatory agent, L is a linker and C is a cell membrane anchor moiety.

159. The method of any one of claims 134 to 158, wherein the cell membrane anchor moiety comprises a CD8a transmembrane-intracellular domain, a B7-1 transmembrane domain, a B7-2 transmembrane domain, or a CD8a transmembrane domain.

160. The method of claim 159, wherein the cell membrane anchor moiety comprises a B7-1 transmembrane domain.

161. The method of claim 160, wherein the cell membrane anchor moiety has the amino acid sequence of SEQ ID NO:239.

162. The method of any one of claims 134 to 161, wherein the immunomodulatory composition comprises two or more different membrane anchored immunomodulatory fusion proteins, wherein each of the different membrane anchored immunomodulatory fusion proteins each comprises a different immunomodulatory agent.

163. The method of claim 162, wherein the different immunomodulatory agents are selected from: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof, and a CD40 agonist.

164. The method of claim 163, wherein the different immunomodulatory agents are selected from: IL-12 and IL-15, IL-15 and IL-18, CD40L and IL-15, IL-15 and IL-21, and IL-2 and IL-12.

165. The method of any one of claims 134 to 164, wherein the modifying comprises introducing a heterologous nucleic acid encoding the fusion protein into the portion of TILs and expressing the fusion protein on the surface of the modified TILs.

166. The method of claim 165, wherein the heterologous nucleic acid comprises an adenoviral vector, a retroviral vector, a lentiviral vector, an adeno-associated vector (AAV), or a piggyBac transposon.

167. The method of claim 165 or 166, wherein the heterologous nucleic acid comprises an NFAT promoter, an EF-1a promotor, an MND promoter, or an SSFV promotor.

168. The method of any of claims 134 to 167, wherein the modified TILs are modified by transfecting the TILs with a nucleic acid encoding the fusion protein.

169. The method of claim 168, wherein the nucleic acid is an RNA.

170. The method of claim 169, wherein the RNA is a mRNA.

171. The method of claim 170, wherein the TILs are transfected with the mRNA by electroporation.

172. The method of claim 171, wherein the TILs are transfected with the mRNA by electroporation after the first expansion and before the second expansion.

173. The method of claim 171, wherein the TILs are transfected with the mRNA by electroporation before the first expansion.

174. The method of claim 168, wherein the modified TILs are transfected with the nucleic acid encoding the fusion protein using a microfluidic device to temporarily disrupt the cell membranes of the TILs, thereby allowing transfection of the nucleic acid.

175. The method of any of claims 171 to 174, wherein the method further comprises activating the TILs by incubation with an anti-CD3 agonist before transfecting the TILs with the mRNA.

176. The method of claim 175, wherein the anti-CD3 agonist is OKT-3.

177. The method of claim 175 or 176, wherein the TILs are activated by incubating the TILs with the anti-CD3 agonist for about 1 to 3 days before transfecting the TILs with the mRNA.

178. A composition comprising the modified TILs of any one of claims 1 to 177 and 180-225.

179. A pharmaceutical composition comprising the modified TILs of any one of claims 1 to 177 and 180 to 225 and a pharmaceutically-acceptable carrier.

180. The method of any of claims 30-60 or 134-179, wherein the one or more membrane anchored immunomodulatory fusion proteins are independently according to the formula, from N- to C-terminus: S1-IA1-L1-C1-L2-S2-IA2-L3-C2, wherein S1 and S2 are each independently a signal peptide, IA1 and IA2 are each independently an immunomodulatory agent, L1-L3 are each independently a linker, and C1 and C2 are each independently a cell membrane anchor moiety.

181. The method of claim 179, wherein S1 and S2 are the same.

182. The method of claim 180 or 181, wherein C1 and C2 are the same.

183. The method of any of 180-182, wherein L2 is a cleavable linker.

184. The method of claim 183, wherein L2 is a furin cleavable linker.

185. The method of any of claims 180 to 184, wherein IA1 and IA2 are each independently a cytokine.

186. The method of any of claims 180 to 184, wherein IA1 and IA2 are each independently selected from the group consisting of: IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof.

187. The method of any of claims 180 to 184, wherein IA1 and IA2 are each independently selected from the group consisting of IL-2 and IL-12, with the proviso that one of IA1 and IA2 is IL-2 and the other is IL-12.

188. The method of any of claims 180 to 184, wherein IA1 and IA2 are each independently selected from the group consisting of IL-15 and IL-21, with the proviso that one of IA1 and IA2 is IL-15 and the other is IL-21.

189. The method of any of claims 1 to 177 or 180 to 188, wherein the modified TILs are genetically modified to express the immunomodulatory composition on the cell surface.

190. The method of claim 189, wherein the immunomodulatory composition comprises one or more membrane anchored immunomodulatory fusion proteins each comprising one or more immunomodulatory agents and a cell membrane anchor moiety.

191. The method of claim 190, wherein the one or more membrane anchored immunomodulatory fusion proteins comprise IL-2 or a variant thereof.

192. The method of claim 190, wherein the one or more membrane anchored immunomodulatory fusion proteins comprise IL-15 or a variant thereof.

193. The method of claim 190, wherein the one or more membrane anchored immunomodulatory fusion proteins comprise IL-18 or a variant thereof.

194. The method of claim 190, wherein the one or more membrane anchored immunomodulatory fusion proteins comprise DR-IL-18.

195. The method of claim 190, wherein the one or more membrane anchored immunomodulatory fusion proteins comprise IL-21 or a variant thereof.

196. The method of claim 190, wherein the modified TILs comprise a first membrane anchored immunomodulatory fusion protein and a second membrane anchored immunomodulatory fusion protein.

197. The method of claim 196, wherein the first membrane anchored immunomodulatory fusion protein comprises IL-15 or a variant thereof, and the second membrane anchored immunomodulatory fusion protein comprises IL-21 or a variant thereof.

198. The method of claim 196 or 197, wherein the first membrane anchored immunomodulatory fusion protein and the second immunomodulatory fusion protein are expressed under the control of an NFAT promoter, an EF-1a promotor, an MND promoter, or an SSFV promotor in the modified TILs.

199. The method of claim 190, wherein the one or more membrane anchored immunomodulatory fusion proteins are independently according to the formula, from N- to C-terminus: S-IA-L-C, wherein S is a signal peptide, IA is an immunomodulatory agent, L is a linker and C is a cell membrane anchor moiety.

200. The method of claim 199, wherein IA is a cytokine.

201. The method of claim 199, wherein IA is selected from the group consisting of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof.

202. The method of claim 199, wherein IA is IL-2 or a variant thereof.

203. The method of claim 199, wherein IA is IL-12 or a variant thereof.

204. The method of claim 199, wherein IA is IL-15 or a variant thereof.

205. The method of claim 199, wherein IA is IL-18 or a variant thereof.

206. The method of claim 199, wherein IA is DR-IL-18.

207. The method of claim 199, wherein IA is IL-21 or a variant thereof.

208. The method of any of claims 199 to 207, wherein L is a CD8a transmembrane-intracellular domain, a B7-1 transmembrane domain, a B7-2 transmembrane domain, or a CD8a transmembrane domain.

209. The method of any of claims 199 to 207, wherein L is a B7-1 transmembrane domain.

210. The method of any of claims 199 to 207, wherein L has the amino acid sequence of SEQ ID NO:239.

211. The method of claim 190, wherein the one or more membrane anchored immunomodulatory fusion proteins are independently according to the formula, from N- to C-terminus: S1-IA1-L1-C1-L2-S2-IA2-L3-C2, wherein S1 and S2 are each independently a signal peptide, IA1 and IA2 are each independently an immunomodulatory agent, L1-L3 are each independently a linker, and C1 and C2 are each independently a cell membrane anchor moiety.

212. The method of claim 211, wherein S1 and S2 are the same.

213. The method of claim 211 or 212, wherein C1 and C2 are the same.

214. The method of any of 211 to 213, wherein L2 is a cleavable linker.

215. The method of claim 214, wherein L2 is a furin cleavable linker.

216. The method of any of claims 211 to 215, wherein IA1 and IA2 are each independently a cytokine.

217. The method of any of claims 211 to 216, wherein IA1 and IA2 are each independently selected from the group consisting of IL-2, IL-6, IL-7, IL-9, IL-12, IL-15, IL-18, IL-21, IL-23, IL-27, IFN gamma, TNFa, IFN alpha, IFN beta, GM-CSF, GCSF, or a variant thereof.

218. The method of any of claims 211 to 216, wherein IA1 and IA2 are each independently selected from the group consisting of IL-2 and IL-12, with the proviso that one of IA1 and IA2 is IL-2 and the other is IL-12.

219. The method of any of claims 211 to 216, wherein IA1 and IA2 are each independently selected from the group consisting of IL-15 and IL-21, with the proviso that one of IA1 and IA2 is IL-15 and the other is IL-21.

220. The method of any of claims 211 to 219, wherein C1 and C2 are each independently a CD8a transmembrane-intracellular domain, a B7-1 transmembrane domain, a B7-2 transmembrane domain, or a CD8a transmembrane domain.

221. The method of any of claims 211 to 219, wherein C1 and C2 are each a B7-1 transmembrane domain.

222. The method of any of claims 211 to 219, wherein C1 and C2 each have the amino acid sequence of SEQ ID NO:239.

223. The method of any of claims 190 to 222, wherein the modified TILs express the one or more membrane anchored immunomodulatory fusion proteins under the control of an NFAT promoter, an EF-1a promotor, an MND promoter, or an SSFV promotor.

224. The method of any of claims 190 to 223, wherein the modified TILs are transduced with a retroviral vector to express the one or more membrane anchored immunomodulatory fusion proteins.

225. The method of any of claims 190 to 223, wherein the modified TILs are transduced with an adenoviral vector, a retroviral vector, a lentiviral vector, an adeno-associated vector (AAV), or a piggyBac transposon to express the one or more membrane anchored immunomodulatory fusion proteins.


SEQUENCE LISTING (402 sequences)

SEQ ID NO: 1 - protein, 476 aa, synthetic construct
SEQIDNOMUROMONABHEAVYCHAINQVQLQQSGAELARPGASVKMSCKASGYTFTRYTMHWVKQRPGQGLEWIGYINPSRGYTNYNQKFKDKATLTTDKSSSTAYMQLSSLTSEDSAVYYCARYYDDHYCLDYWGQGTTLTVSSAKTTAPSVYPLAPVCGGTTGSSVTLGCLVKGYFPEPVTLTWNSGSLSSGVHTFPAVLQSDLYTLSSSVTVTSSTWPSQSITCNVAHPASSTKVDKKIEPRPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 213 aa, synthetic construct
QIVLTQSPAIMSASPGEKVTMTCSASSSVSYMNWYQQKSGTSPKRWIYDTSKLASGVPAHFRGSGSGTSYSLTISGMEAEDAATYYCQQWSSNPFTFGSGTKLEINRADTAPTVSIFPPSSEQLTSGGASVVCFLNNFYPKDINVKWKIDGSERQNGVLNSWTDQDSKDSTYSMSSTLTLTKDEYERHNSYTCEATHKTSTSPIVKSFNRNEC

SEQ ID NO: 3 - protein, 134 aa, synthetic construct
MAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 4 - protein, 132 aa, synthetic construct
PTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFSQSIISTLT

SEQ ID NO: 5 - protein, 133 aa, synthetic construct [sequence omitted]

SEQ ID NO: 6 - protein, 303 aa, synthetic construct
SKNFHLRPRDLISNINVIVLELKGSETTFMCEYADETATIVEFLNRWITFSQSIISTLTGGSSSTKKTQLQLEHLLLDLQMILNGINNYKNPKLTRMLTFKFYMPKKATELKHLQCLEEELKPLEEVLNLAQGSGGGSELCDDDPPEIPHATFKAMAYKEGTMLNCECKRGFRRIKSGSLYMLCTGNSSHSSWDNQCQCTSSATRNTTKQVTPQPEEQKERKTTEMQSPMQPVDQASLPGHCREPPPWENEATERIYHFVVGQMVYYQCVQGYRALHRGPAESVCKMTHGKTRWTQPQLICTG

SEQ ID NO: 7 - protein, 452 aa, synthetic construct [sequence omitted]

SEQ ID NO: 8 - protein, 16 aa, synthetic construct
SESSASSDGPHPVITP

SEQ ID NO: 9 - protein, 130 aa, synthetic construct
MHKCDITLQEIIKTLNSLTEQKTLCTELTVTDIFAASKNTTEKETFCRAATVLRQFYSHHEKDTRCLGATAQQFHRHKQLIRFLKRLDRNLWGLAGLNSCPVKEANQSTLENFLERLKTIMREKYSKCSS

SEQ ID NO: 10 - protein, 153 aa, synthetic construct
MDCDIEGKDGKQYESVLMVSIDQLLDSMKEIGSNCLNNEFNFFKRHICDANKEGMFLFRAARKLRQFLKMNSTGDFDLHLLKVSEGTTILLNCTGQVKGRKPAALGEAQPTKSLEENKSLKEQKKLNDLCFLKRLLQEIKTCWNKILMGTKEH

SEQ ID NO: 11 - protein, 115 aa, synthetic construct
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 12 - protein, 132 aa, synthetic construct
MQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDS

SEQ ID NO: 13 - protein, 153 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 14 - protein, 133 aa, synthetic construct
APTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 15 - protein, 133 aa, synthetic construct
APTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 16 - protein, 145 aa, synthetic construct
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVG

SEQ ID NO: 17 - protein, 16 aa, synthetic construct
DIWWDDKKDYNPSLKS

SEQ ID NO: 18 - protein, 10 aa, synthetic construct
SMITNWYFDV

SEQ ID NO: 19 - protein, 141 aa, synthetic construct
APTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVG

SEQ ID NO: 20 - protein, 16 aa, synthetic construct
DIWWDDKKDYNPSLKS

SEQ ID NO: 21 - protein, 10 aa, synthetic construct
SMITNWYFDV

SEQ ID NO: 22 - protein, 142 aa, synthetic construct
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGM

SEQ ID NO: 23 - protein, 5 aa, synthetic construct
WWDDK

SEQ ID NO: 24 - protein, 10 aa, synthetic construct
SMITNWYFDV

SEQ ID NO: 25 - protein, 143 aa, synthetic construct
GFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMS

SEQ ID NO: 26 - protein, 7 aa, synthetic construct
IWWDDKK

SEQ ID NO: 27 - protein, 12 aa, synthetic construct
ARSMITNWYFDV

SEQ ID NO: 28 - protein, 253 aa, synthetic construct
QVTLRESGPALVKPTQTLTLTCTFSGFSLAPTSSSTKKTQLQLEHLLLDLQMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSS

SEQ ID NO: 29 - protein, 533 aa, synthetic construct
QMILNGINNYKNPKLTAMLTFKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 10 aa, synthetic construct
KAQLSVGYMH

SEQ ID NO: 31 - protein, 7 aa, synthetic construct
DTSKLAS

SEQ ID NO: 32 - protein, 9 aa, synthetic construct
FQGSGYPFT

SEQ ID NO: 33 - protein, 6 aa, synthetic construct
QLSVGY

SEQ ID NO: 34 - skipped

SEQ ID NO: 35 - protein, 6 aa, synthetic construct
GSGYPF

SEQ ID NO: 36 - protein, 106 aa, synthetic construct
DIQMTQSPSTLSASVGDRVTITCKAQLSVGYMHWYQQKPGKAPKLLIYDTSKLASGVPSRFSGSGSGTEFTLTISSLQPDDFATYYCFQGSGYPFTFGGGTKLEIK

SEQ ID NO: 37 - protein, 213 aa, synthetic construct
DIQMTQSPSTLSASVGDRVTITCKAQLSVGYMHWYQQKPGKAPKLLIYDTSKLASGVPSRFSGSGSGTEFTLTISSLQPDDFATYYCFQGSGYPFTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 38 - protein, 583 aa, synthetic construct
QVTLRESGPALVKPTQTLTLTCTFSGFSLAPTSSSTKKTQLQLEHLLLDLQMILNGINNYKNPKLTRMLTAKFYMPKKATELKHLQCLEEELKPLEEVLNLAQSKNFHLRPRDLISNINVIVLELKGSETTFMCEYADETATIVEFLNRWITFCQSIISTLTSTSGMSVGWIRQPPGKALEWLADIWWDDKKDYNPSLKSRLTISKDTSKNQVVLKVTNMDPADTATYYCARSMITNWYFDVWGAGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVAVSHEDPEVKFNWYVDGVEVHNAKTKPREEQYNSTYRVVSVLTVLHQDWLNGKEYKCKVSNKALAAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 39 - protein, 17 aa, synthetic construct
SEQIDNOLIGHTCHAIN

SEQ ID NO: 40 - protein, 255 aa, synthetic construct
MGNSCYNIVATLLLVLNFERTRSLQDPCSNCPAGTFCDNNRNQICSPCPPNSFSSAGGQRTCDICRQCKGVFRTRKECSSTSNAECDCTPGFHCLGAGCSMCEQDCKQGQELTKKGCKDCCFGTFNDQKRGICRPWTNCSLDGKSVLVNGTKERDVVCGPSPADLSPGASSVTPPAPAREPGHSPQIISFFLALTSTALLFLLFFLTLRFSVVKRGRKKLLYIFKQPFMRPVQTTQEEDGCSCRFPEEEEGGCEL

SEQ ID NO: 41 - protein, 256 aa, synthetic construct
MGNNCYNVVVIVLLLVGCEKVGAVQNSCDNCQPGTFCRKYNPVCKSCPPSTFSSIGGQPNCNICRVCAGYFRFKKFCSSTHNAECECIEGFHCLGPQCTRCEKDCRPGQELTKQGCKTCSLGTFNDQNGTGVCRPWTNCSLDGRSVLKTGTTEKDVVCGPPVVSFSPSTTISVTPEGGPGGHSLQVLTLFLALTSALLLALIFITLLFSVLKWIRKKFPHIFKQPFKKTTGAAQEEDACSCRCPQEEEGGGGGYEL

SEQ ID NO: 42 - protein, 441 aa, synthetic construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPG

SEQ ID NO: 43 - protein, 214 aa, synthetic construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 44 - protein, 116 aa, synthetic construct
EVQLVQSGAEVKKPGESLRISCKGSGYSFSTYWISWVRQMPGKGLEWMGKIYPGDSYTNYSPSFQGQVTISADKSISTAYLQWSSLKASDTAMYYCARGYGIFDYWGQGTLVTVSS

SEQ ID NO: 45 - protein, 108 aa, synthetic construct
SYELTQPPSVSVSPGQTASITCSGDNIGDQYAHWYQQKPGQSPVLVIYQDKNRPSGIPERFSGSNSGNTATLTISGTQAMDEADYYCATYTGFGSLAVFGGGTKLTVL

SEQ ID NO: 46 - protein, 6 aa, synthetic construct
STYWIS

SEQ ID NO: 47 - protein, 17 aa, synthetic construct
KIYPGDSYTNYSPSFQG

SEQ ID NO: 48 - protein, 8 aa, synthetic construct
RGYGIFDY

SEQ ID NO: 49 - protein, 11 aa, synthetic construct
SGDNIGDQYAH

SEQ ID NO: 50 - protein, 7 aa, synthetic construct
QDKNRPS

SEQ ID NO: 51 - protein, 11 aa, synthetic construct
ATYTGFGSLAV

SEQ ID NO: 52 - protein, 448 aa, synthetic construct
QVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGPGNYDWYFDLWGRGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 53 - protein, 216 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPALTFCGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 54 - protein, 120 aa, synthetic construct
MKHLWFFLLLVAAPRWVLSQVQLQQWGAGLLKPSETLSLTCAVYGGSFSGYYWSWIRQSPEKGLEWIGEINHGGYVTYNPSLESRVTISVDTSKNQFSLKLSSVTAADTAVYYCARDYGP

SEQ ID NO: 55 - protein, 110 aa, synthetic construct
MEAPAQLLFLLLLWLPDTTGEIVLTQSPATLSLSPGERATLSCRASQSVSSYLAWYQQKPGQAPRLLIYDASNRATGIPARFSGSGSGTDFTLTISSLEPEDFAVYYCQQ

SEQ ID NO: 56 - protein, 5 aa, synthetic construct
GYYWS

SEQ ID NO: 57 - protein, 16 aa, synthetic construct
EINHGGYVTYNPSLES

SEQ ID NO: 58 - protein, 13 aa, synthetic construct
DYGPGNYDWYFDL

SEQ ID NO: 59 - protein, 11 aa, synthetic construct
RASQSVSSYLA

SEQ ID NO: 60 - protein, 7 aa, synthetic construct
DASNRAT

SEQ ID NO: 61 - protein, 11 aa, synthetic construct
QQRSDWPPALT

SEQ ID NO: 62 - protein, 230 aa, synthetic construct
KSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 63 - protein, 22 aa, synthetic construct
GGPGSSKSCDKTHTCPPCPAPE

SEQ ID NO: 64 - protein, 22 aa, synthetic construct
GGSGSSKSCDKTHTCPPCPAPE

SEQ ID NO: 65 - protein, 27 aa, synthetic construct
GGPGSSSSSSSKSCDKTHTCPPCPAPE

SEQ ID NO: 66 - protein, 27 aa, synthetic construct
GGSGSSSSSSSKSCDKTHTCPPCPAPE

SEQ ID NO: 67 - protein, 29 aa, synthetic construct
GGPGSSSSSSSSSKSCDKTHTCPPCPAPE

SEQ ID NO: 68 - protein, 29 aa, synthetic construct
GGSGSSSSSSSSSKSCDKTHTCPPCPAPE

SEQ ID NO: 69 - protein, 24 aa, synthetic construct
GGPGSSGSGSSDKTHTCPPCPAPE

SEQ ID NO: 70 - protein, 23 aa, synthetic construct
GGPGSSGSGSDKTHTCPPCPAPE

SEQ ID NO: 71 - protein, 21 aa, synthetic construct
GGPSSSGSDKTHTCPPCPAPE

SEQ ID NO: 72 - protein, 25 aa, synthetic construct
GGSSSSSSSSGSDKTHTCPPCPAPE

SEQ ID NO: 73 - protein, 246 aa, synthetic construct
METDTLLLWVLLLWVPAGNGDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 74 - protein, 11 aa, synthetic construct
SGSGSGSGSGS

SEQ ID NO: 75 - protein, 12 aa, synthetic construct
SSSSSSGSGSGS

SEQ ID NO: 76 - protein, 16 aa, synthetic construct
SSSSSSGSGSGSGSGS

SEQ ID NO: 77 - protein, 254 aa, synthetic construct
MEYASDASLDPEAPWPPAPRARACRVLPWALVAGLLLLLLLAAACAVFLACPWAVSGARASPGSAASPRLREGPELSPDDPAGLLDLRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 78 - protein, 168 aa, synthetic construct
LRQGMFAQLVAQNVLLIDGPLSWYSDPGLAGVSLTGGLSYKEDTKELVVAKAGVYYVFFQLELRRVVAGEGSGSVSLALHLQPLRSAAGAAALALTVDLPPASSEARNSAFGFQGRLLHLSAGQRLGVHLHTEARARHAWQLTQGATVLGLFRVTPEIPAGLPSPRSE

SEQ ID NO: 79 - protein, 118 aa, synthetic construct
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVS

SEQ ID NO: 80 - protein, 107 aa, synthetic construct
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIK

SEQ ID NO: 81 - protein, 119 aa, synthetic construct
QVQLQQPGAELVKPGASVKLSCKASGYTFSSYWMHWVKQRPGQVLEWIGEINPGNGHTNYNEKFKSKATLTVDKSSSTAYMQLSSLTSEDSAVYYCARSFTTARGFAYWGQGTLVTVSA

SEQ ID NO: 82 - protein, 108 aa, synthetic construct
DIVMTQSPATQSVTPGDRVSLSCRASQTISDYLHWYQQKSHESPRLLIKYASQSISGIPSRFSGSGSGSDFTLSINSVEPEDVGVYYCQDGHSFPPTFGGGTKLEIKR

SEQ ID NO: 83 - protein, 120 aa, synthetic construct
MDWTWRILFLVAAATGAHSEVQLVESGGGLVQPGGSLRLSCAASGFTFSDYWMSWVRQAPGKGLEWVADIKNDGSYTNYAPSLTNRFTISRDNAKNSLYLQMNSLRAEDTAVYYCARELT

SEQ ID NO: 84 - protein, 109 aa, synthetic construct
MEAPAQLLFLLLLWLPDTTGDIVMTQSPDSLAVSLGERATINCKSSQSLLSSGNQKNYLWYQQKPGQPPKLLIYYASTRQSGVPDRFSGSGSGTDFTLTISSLQAEDVA

SEQ ID NO: 85 - protein, 277 aa, synthetic construct
MCVGARRLGRGPCAALLLLGLGLSTVTGLHCVGDTYPSNDRCCHECRPGNGMVSRCSRSQNTVCRPCGPGFYNDVVSSKPCKPCTWCNLRSGSERKQLCTATQDTVCRCRAGTQPLDSYKPGVDCAPCPPGHFSPGDNQACKPWTNCTLAGKHTLQPASNSSDAICEDRDPPATQPQETQGPPARPITVQPTEAWPRTSQGPSTRPVEVPGGRAVAAILGLGLVLGLLGPLAILLALYLLRRDQRLPPDAHKPPGGGSFRTPIQEEQADAHSTLAKI

SEQ ID NO: 86 - protein, 272 aa, synthetic construct
MYVWVQQPTALLLLGLTLGVTARRLNCVKHTYPSGHKCCRECQPGHGMVSRCDHTRDTLCHPCETGFYNEAVNYDTCKQCTQCNHRSGSELKQNCTPTQDTVCRCRPGTQPRQDSGYKLGVDCVPCPPGHFSPGNNQACKPWTNCTLSGKQTRHPASDSLDAVCEDRSLLATLLWETQRPTFRPTTVQSTTVWPRTSELPSPPTLVTPEGPAFAVLLGLGLGLLAPLTVLLALYLLRKAWRLPNTPKPCWGNSFRTPIQEEHTDAHFTLAKI

SEQ ID NO: 87 - protein, 451 aa, synthetic construct
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 88 - protein, 214 aa, synthetic construct
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 89 - protein, 118 aa, synthetic construct
QVQLQESGPGLVKPSQTLSLTCAVYGGSFSSGYWNWIRKHPGKGLEYIGYISYNGITYHNPSLKSRITINRDTSKNQYSLQLNSVTPEDTAVYYCARYKYDYDGGHAMDYWGQGTLVT

SEQ ID NO: 90 - protein, 108 aa, synthetic construct
DIQMTQSPSSLSASVGDRVTITCRASQDISNYLNWYQQKPGKAPKLLIYYTSKLHSGVPSRFSGSGSGTDYTLTISSLQPEDFATYYCQQGSALPWTFGQGTKVEIKR

SEQ ID NO: 91 - protein, 9 aa, synthetic construct
GSFSSGYWN

SEQ ID NO: 92 - protein, 13 aa, synthetic construct
YIGYISYNGITYH

SEQ ID NO: 93 - protein, 14 aa, synthetic construct
RYKYDYDGGHAMDY

SEQ ID NO: 94 - protein, 8 aa, synthetic construct
QDISNYLN

SEQ ID NO: 95 - protein, 11 aa, synthetic construct
LLIYYTSKLHS

SEQ ID NO: 96 - protein, 8 aa, synthetic construct
QQGSALPW

SEQ ID NO: 97 - protein, 444 aa, synthetic construct
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 98 - protein, 214 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 99 - protein, 118 aa, synthetic construct
EVQLVESGGGLVQPGGSLRLSCAASGFTFSSYSMNWVRQAPGKGLEWVSYISSSSSTIDYADSVKGRFTISRDNAKNSLYLQMNSLRDEDTAVYYCARESGWYLFDYWGQGTLVTVSS

SEQ ID NO: 100 - protein, 107 aa, synthetic construct
DIQMTQSPSSLSASVGDRVTITCRASQGISSWLAWYQQKPEKAPKSLIYAASSLQSGVPSRFSGSGSGTDFTLTISSLQPEDFATYYCQQYNSYPPTFGGGTKVEIK

SEQ ID NO: 101 - protein, 5 aa, synthetic construct
SYSMN

SEQ ID NO: 102 - protein, 17 aa, synthetic construct
YISSSSSTIDYADSVKG

SEQ ID NO: 103 - protein, 33 aa, synthetic construct
SEQIDNOHEAVYCHAINCDRFORDESGWYLFDY

SEQ ID NO: 104 - protein, 11 aa, synthetic construct
RASQGISSWLA

SEQ ID NO: 105 - protein, 7 aa, synthetic construct
AASSLQS

SEQ ID NO: 106 - protein, 9 aa, synthetic construct
QQYNSYPPT

SEQ ID NO: 107 - protein, 450 aa, synthetic construct
EVQLVESGGGLVQPGRSLRLSCAASGFTFDDYAMHWVRQAPGKGLEWVSGISWNSGSIGYADSVKGRFTISRDNAKNSLYLQMNSLRAEDTALYYCAKDQSTADYYFYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 108 - protein, 213 aa, synthetic construct
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 109 - protein, 35 aa, synthetic construct
SEQIDNOHEAVYCHAINVARIABLEREGIONFORD

SEQ ID NO: 110 - protein, 106 aa, synthetic construct
EIVVTQSPATLSLSPGERATLSCRASQSVSSYLAWYQQKPGQAPRLLIYDASNRATGIPARFSGSGSGTDFTLTISSLEPEDFAVYYCQQRSNWPTFGQGTKVEIK

SEQ ID NO: 111 - protein, 5 aa, synthetic construct
DYAMH

SEQ ID NO: 112 - protein, 17 aa, synthetic construct
GISWNSGSIGYADSVKG

SEQ ID NO: 113 - protein, 15 aa, synthetic construct
DQSTADYYFYYGMDV

SEQ ID NO: 114 - protein, 11 aa, synthetic construct
RASQSVSSYLA

SEQ ID NO: 115 - protein, 7 aa, synthetic construct
DASNRAT

SEQ ID NO: 116 - protein, 8 aa, synthetic construct
QQRSNWPT

SEQ ID NO: 117 - protein, 120 aa, synthetic construct
EVQLVESGGGLVQPGGSLRLSCAASEYEFPSHDMSWVRQAPGKGLELVAAINSDGGSTYYPDTMERRFTISRDNAKNSLYLQMNSLRAEDTAVYYCARHYDDYYAWFAYWGQGTMVTVSS

SEQ ID NO: 118 - protein, 111 aa, synthetic construct
EIVLTQSPATLSLSPGERATLSCRASKSVSTSGYSYMHWYQQKPGQAPRLLIYLASNLESGVPARFSGSGSGTDFTLTISSLEPEDFAVYYCQHSRELPLTFGGGTKVEIK

SEQ ID NO: 119 - protein, 5 aa, synthetic construct
SHDMS

SEQ ID NO: 120 - protein, 17 aa, synthetic construct
AINSDGGSTYYPDTMER

SEQ ID NO: 121 - protein, 11 aa, synthetic construct
HYDDYYAWFAY

SEQ ID NO: 122 - protein, 15 aa, synthetic construct
RASKSVSTSGYSYMH

SEQ ID NO: 123 - protein, 7 aa, synthetic construct
LASNLES

SEQ ID NO: 124 - protein, 9 aa, synthetic construct
QHSRELPLT

SEQ ID NO: 125 - protein, 122 aa, synthetic construct
QVQLVQSGSELKKPGASVKVSCKASGYTFTDYSMHWVRQAPGQGLKWMGWINTETGEPTYADDFKGRFVFSLDTSVSTAYLQISSLKAEDTAVYYCANPYYDYVSYYAMDYWGQGTTVTVSS

SEQ ID NO: 126 - protein, 107 aa, synthetic construct
DIQMTQSPSSLSASVGDRVTITCKASQDVSTAVAWYQQKPGKAPKLLIYSASYLYTGVPSRFSGSGSGTDFTFTISSLQPEDIATYYCQQHYSTPRTFGQGTKLEIK

SEQ ID NO: 127 - protein, 5 aa, synthetic construct
DYSMH

SEQ ID NO: 128 - protein, 17 aa, synthetic construct
WINTETGEPTYADDFKG

SEQ ID NO: 129 - protein, 13 aa, synthetic construct
PYYDYVSYYAMDY

SEQ ID NO: 130 - protein, 11 aa, synthetic construct
KASQDVSTAVA

SEQ ID NO: 131 - protein, 7 aa, synthetic construct
SASYLYT

SEQ ID NO: 132 - protein, 9 aa, synthetic construct
QQHYSTPRT

SEQ ID NO: 133 - protein, 183 aa, synthetic construct
MERVQPLEENVGNAARPRFERNKLLLVASVIQGLGLLLCFTYICLHFSALQVSHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 134 - protein, 131 aa, synthetic construct
SHRYPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 135 - protein, 128 aa, synthetic construct
YPRIQSIKVQFTEYKKEKGFILTSQKEDEIMKVQNNSVIINCDGFYLISLKGYFSQEVNISLHYQKDEEPLFQLKKVRSVNSLMVASLTYKDKVYLNVTTDNTSLDDFHVNGGELILIHQNPGEFCVL

SEQ ID NO: 136 - protein, 120 aa, synthetic construct
EVQLVESGGGLVQPGGSLRLSCAASGFTFSNYTMNWVRQAPGKGLEWVSAISGSGGSTYYADSVKGRFTISRDNSKNTLYLQMNSLRAEDTAVYYCAKDRYSQVHYALDYWGQGTLVTVS

SEQ ID NO: 137 - protein, 108 aa, synthetic construct
DIVMTQSPDSLPVTPGEPASISCRSSQSLLHSNGYNYLDWYLQKAGQSPQLLIYLGSNRASGVPDRFSGSGSGTDFTLKISRVEAEDVGVYYCQQYYNHPTTFGQGTK

SEQ ID NO: 138 - protein, 120 aa, synthetic construct
EVQLVESGGGVVQPGRSLRLSCAASGFTFSDYTMNWVRQAPGKGLEWVSSISGGSTYYADSRKGRFTISRDNSKNTLYLQMNNLRAEDTAVYYCARDRYFRQQNAFDYWGQGTLVTVSSA

SEQ ID NO: 139 - protein, 108 aa, synthetic construct; identical to SEQ ID NO: 137

SEQ ID NO: 140 - protein, 120 aa, synthetic construct
EVQLVESGGGLVQPRGSLRLSCAASGFTFSSYAMNWVRQAPGKGLEWVAVISYDGSNKYYADSVKGRFTISRDNSKNTLYLQMNSLRAEDTAVYYCAKDRYITLPNALDYWGQGTLVTVS

SEQ ID NO: 141 - protein, 108 aa, synthetic construct
DIQMTQSPVSLPVTPGEPASISCRSSQSLLHSNGYNYLDWYLQKPGQSPQLLIYLGSNRASGVPDRFSGSGSGTDFTLKISRVEAEDVGVYYCQQYKSNPPTFGQGTK

SEQ ID NO: 142 - protein, 120 aa, synthetic construct
EVQLVESGGGLVHPGGSLRLSCAGSGFTFSSYAMHWVRQAPGKGLEWVSAIGTGGGTYYADSVMGRFTISRDNSKNTLYLQMNSLRAEDTAVYYCARYDNVMGLYWFDYWGQGTLVTVSS

SEQ ID NO: 143 - protein, 108 aa, synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPPAFGGGTKVEIKR

SEQ ID NO: 144 - protein, 31 aa, synthetic construct
SEQIDNOHEAVYCHAINVARIABLEREGION

SEQ ID NO: 145 - protein, 108 aa, synthetic construct
DIQMTQTTSSLSASLGDRVTISCRASQDISNYLNWYQQKPDGTVKLLIYYTSRLHSGVPSRFSGSGSGTDYSLTISNLEQEDIATYFCQQGNTLPWTFGGGTKLEIKR

SEQ ID NO: 146 - protein, 121 aa, synthetic construct
EVQLQQSGPELVKPGASVKISCKTSGYTFKDYTMHWVKQSHGKSLEWIGGIYPNNGGSTYNQNFKDKATLTVDKSSSTAYMEFRSLTSEDSAVYYCARMGYHGPHLDFDVWGAGTTVTVSP

SEQ ID NO: 147 - protein, 108 aa, synthetic construct
DIVMTQSHKFMSTSLGDRVSITCKASQDVGAAVAWYQQKPGQSPKLLIYWASTRHTGVPDRFTGGGSGTDFTLTISNVQSEDLTDYFCQQYINYPLTFGGGTKLEIKR

SEQ ID NO: 148 - protein, 122 aa, synthetic construct
QIQLVQSGPELKKPGETVKISCKASGYTFTDYSMHWVKQAPGKGLKWMGWINTETGEPTYADDFKGRFAFSLETSASTAYLQINNLKNEDTATYFCANPYYDYVSYYAMDYWGHGTSVTVSS

SEQ ID NO: 149 - protein, 122 aa, synthetic construct; identical to SEQ ID NO: 125

SEQ ID NO: 150 - protein, 107 aa, synthetic construct
DIVMTQSHKFMSTSVRDRVSITCKASQDVSTAVAWYQQKPGQSPKLLIYSASYLYTGVPDRFTGSGSGTDFTFTISSVQAEDLAVYYCQQHYSTPRTFGGGTKLEIK

SEQ ID NO: 151 - protein, 107 aa, synthetic construct; identical to SEQ ID NO: 150

SEQ ID NO: 152 - protein, 120 aa, synthetic construct
EVQLVESGGGLVQPGESLKLSCESNEYEFPSHDMSWVRKTPEKRLELVAAINSDGGSTYYPDTMERRFIISRDNTKKTLYLQMSSLRSEDTALYYCARHYDDYYAWFAYWGQGTLVTVSA

SEQ ID NO: 153 - protein, 120 aa, synthetic construct; identical to SEQ ID NO: 117

SEQ ID NO: 154 - protein, 111 aa, synthetic construct
DIVLTQSPASLAVSLGQRATISCRASKSVSTSGYSYMHWYQQKPGQPPKLLIYLASNLESGVPARFSGSGSGTDFTLNIHPVEEEDAATYYCQHSRELPLTFGAGTKLELK

SEQ ID NO: 155 - protein, 111 aa, synthetic construct; identical to SEQ ID NO: 118

SEQ ID NO: 156 - protein, 138 aa, synthetic construct
MYLGLNYVFIVFLLNGVQSEVKLEESGGGLVQPGGSMKLSCAASGFTFSDAWMDWVRQSPEKGLEWVAEIRSKANNHATYYAESVNGRFTISRDDSKSSVYLQMNSLRAEDTGIYYCTWGEVFYFDYWGQGTTLTVSS

SEQ ID NO: 157 - protein, 126 aa, synthetic construct
MRPSIQFLGLLLFWLHGAQCDIQMTQSPSSLSASLGGKVTITCKSSQDINKYIAWYQHKPGKGPRLLIHYTSTLQPGIPSRFSGSGSGRDYSFSISNLEPEDIATYYCLQYDNLLTFGAGTKLELK

SEQ ID NO: 158 - protein, 440 aa, synthetic construct
QVQLVESGGGVVQPGRSLRLDCKASGITFSNSGMHWVRQAPGKGLEWVAVIWYDGSKRYYADSVKGRFTISRDNSKNTLFLQMNSLRAEDTAVYYCATNDDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 159 - protein, 26 aa, synthetic construct
SEQIDNONIVOLUMABLIGHTCHAIN

SEQ ID NO: 160 - protein, 113 aa, synthetic construct [sequence omitted]

SEQ ID NO: 161 - protein, 107 aa, synthetic construct [sequence omitted]

SEQ ID NO: 162 - protein, 5 aa, synthetic construct
NSGMH

SEQ ID NO: 163 - protein, 17 aa, synthetic construct
VIWYDGSKRYYADSVKG

SEQ ID NO: 164 - protein, 4 aa, synthetic construct
NDDY

SEQ ID NO: 165 - protein, 11 aa, synthetic construct
RASQSVSSYLA

SEQ ID NO: 166 - protein, 7 aa, synthetic construct
DASNRAT

SEQ ID NO: 167 - protein, 9 aa, synthetic construct
QQSSNWPRT

SEQ ID NO: 168 - protein, 447 aa, synthetic construct
QVQLVQSGVEVKKPGASVKVSCKASGYTFTNYYMYWVRQAPGQGLEWMGGINPSNGGTNFNEKFKNRVTLTTDSSTTTAYMELKSLQFDDTAVYYCARRDYRFDMGFDYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 169 - protein, 218 aa, synthetic construct
EIVLTQSPATLSLSPGERATLSCRASKGVSTSGYSYLHWYQQKPGQAPRLLIYLASYLESGVPARFSGSGSGTDFTLTISSLEPEDFAVYYCQHSRDLPLTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 170 - protein, 120 aa, synthetic construct [sequence omitted]

SEQ ID NO: 171 - protein, 111 aa, synthetic construct [sequence omitted]

SEQ ID NO: 172 - protein, 111 aa, synthetic construct [sequence omitted]

SEQ ID NO: 173 - protein, 16 aa, synthetic construct
GINPSNGGTNFNEKFK

SEQ ID NO: 174 - protein, 11 aa, synthetic construct
RDYRFDMGFDY

SEQ ID NO: 175 - protein, 15 aa, synthetic construct
RASKGVSTSGYSYLH

SEQ ID NO: 176 - protein, 7 aa, synthetic construct
LASYLES

SEQ ID NO: 177 - protein, 9 aa, synthetic construct
QHSRDLPLT

SEQ ID NO: 178 - protein, 451 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEFEGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPASIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 179 - protein, 265 aa, synthetic construct
EVQLVESGGGLVQPGGSLRLSCAASGFTFSRYWMSWVRQAPGKGLEWVANEIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 180 - protein, 121 aa, synthetic construct
EVQLVESGGGLVQPGGSLRLSCAASGFTFSRYWMSWVRQAPGKGLEWVANIKQDGSEKYYVDSVKGRFTISRDNAKNSLYLQMNSLRAEDTAVYYCAREGGWFGELAFDYWGQGTLVTVSS

SEQ ID NO: 181 - protein, 108 aa, synthetic construct
EIVLTQSPGTLSLSPGERATLSCRASQRVSSSYLAWYQQKPGQAPRLLIYDASSRATGIPDRFSGSGSGTDFTLTISRLEPEDFAVYYCQQYGSLPWTFGQGTKVEIK

SEQ ID NO: 182 - protein, 5 aa, synthetic construct
RYWMS

SEQ ID NO: 183 - protein, 17 aa, synthetic construct
NIKQDGSEKYYVDSVKG

SEQ ID NO: 184 - protein, 12 aa, synthetic construct
EGGWFGELAFDY

SEQ ID NO: 185 - protein, 12 aa, synthetic construct
RASQRVSSSYLA

SEQ ID NO: 186 - protein, 7 aa, synthetic construct
DASSRAT

SEQ ID NO: 187 - protein, 9 aa, synthetic construct
QQYGSLPWT

SEQ ID NO: 188 - protein, 450 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 189 - protein, 216 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVLGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 190 - protein, 120 aa, synthetic construct
EVQLLESGGGLVQPGGSLRLSCAASGFTFSSYIMMWVRQAPGKGLEWVSSIYPSGGITFYADTVKGRFTISRDNSKNTLYLQMNSLRAEDTAVYYCARIKLGTVTTVDYWGQGTLVTVSS

SEQ ID NO: 191 - protein, 110 aa, synthetic construct
QSALTQPASVSGSPGQSITISCTGTSSDVGGYNYVSWYQQHPGKAPKLMIYDVSNRPSGVSNRFSGSKSGNTASLTISGLQAEDEADYYCSSYTSSSTRVFGTGTKVTVL

SEQ ID NO: 192 - protein, 5 aa, synthetic construct
SYIMM

SEQ ID NO: 193 - protein, 17 aa, synthetic construct
SIYPSGGITFYADTVKG

SEQ ID NO: 194 - protein, 11 aa, synthetic construct
IKLGTVTTVDY

SEQ ID NO: 195 - protein, 14 aa, synthetic construct
TGTSSDVGGYNYVS

SEQ ID NO: 196 - protein, 7 aa, synthetic construct
DVSNRPS

SEQ ID NO: 197 - protein, 10 aa, synthetic construct
SSYTSSSTRV

SEQ ID NO: 198 - protein, 448 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYASTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 199 - protein, 214 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 200 - protein, 118 aa, synthetic construct
EVQLVESGGGLVQPGGSLRLSCAASGFTFSDSWIHWVRQAPGKGLEWVAWISPYGGSTYYADSVKGRFTISADTSKNTAYLQMNSLRAEDTAVYYCARRHWPGGFDYWGQGTLVTVSA

SEQ ID NO: 201 - protein, 108 aa, synthetic construct
DIQMTQSPSSLSASVGDRVTITCRASQDVSTAVAWYQQKPGKAPKLLIYSASFLYSGVPSRFSGSGSGTDFTLTISSLQPEDFATYYCQQYLYHPATFGQGTKVEIKR

SEQ ID NO: 202 - protein, 10 aa, synthetic construct
GFTFSDSWIH

SEQ ID NO: 203 - protein, 18 aa, synthetic construct
AWISPYGGSTYYADSVKG

SEQ ID NO: 204 - protein, 9 aa, synthetic construct
RHWPGGFDY

SEQ ID NO: 205 - protein, 11 aa, synthetic construct
RASQDVSTAVA

SEQ ID NO: 206 - protein, 7 aa, synthetic construct
SASFLYS

SEQ ID NO: 207 - protein, 9 aa, synthetic construct
QQYLYHPAT

SEQ ID NO: 208 - protein, 225 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTH

SEQ ID NO: 209 - protein, 215 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 210 - protein, 118 aa, synthetic construct
QVQLVESGGGVVQPGRSLRLSCAASGFTFSSYTMHWVRQAPGKGLEWVTFISYDGNNKYYADSVKGRFTISRDNSKNTLYLQMNSLRAEDTAIYYCARTGWLGPFDYWGQGTLVTVSS

SEQ ID NO: 211 - protein, 108 aa, synthetic construct
EIVLTQSPGTLSLSPGERATLSCRASQSVGSSYLAWYQQKPGQAPRLLIYGAFSRATGIPDRFSGSGSGTDFTLTISRLEPEDFAVYYCQQYGSSPWTFGQGTKVEIK

SEQ ID NO: 212 - protein, 8 aa, synthetic construct
GFTFSSYT

SEQ ID NO: 213 - protein, 10 aa, synthetic construct
TFISYDGNNK

SEQ ID NO: 214 - protein, 11 aa, synthetic construct
ARTGWLGPFDY

SEQ ID NO: 215 - protein, 7 aa, synthetic construct
QSVGSSY

SEQ ID NO: 216 - skipped

SEQ ID NO: 217 - protein, 9 aa, synthetic construct
QQYGSSPWT

SEQ ID NO: 218 - protein, 451 aa, synthetic construct
QVQLVESGGGVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTVERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 219 - protein, 214 aa, synthetic construct
DIQMTQSPSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 220 - protein, 167 aa, synthetic construct
GVVQPGRSLRLSCAASGFTFSSYGMHWVRQAPGKGLEWVAVIWYDGSNKYYADSVKGRFTISRDNSKNTLYLQMNSLRAEDTAVYYCARDPRGATLYYYYYGMDVWGQGTTVTVSSASTKGPSVFPLAPCSRSTSESTAALGCLVKDYFPEPVTVSWNSGALTSGVH

SEQ ID NO: 221 - protein, 139 aa, synthetic construct
PSSLSASVGDRVTITCRASQSINSYLDWYQQKPGKAPKLLIYAASSLQSGVPSRFSGSGSGTDFTLTISSLQPEDFATYYCQQYYSTPFTFGPGTKVEIKRTVAAPSVFIFPPSDEQLKSGTASVVCLLNNFYPREAKV

SEQ ID NO: 222 - protein, 10 aa, synthetic construct
GFTFSSYGMH

SEQ ID NO: 223 - protein, 15 aa, synthetic construct
VIWYDGSNKYYADSV

SEQ ID NO: 224 - protein, 16 aa, synthetic construct
DPRGATLYYYYYGMDV

SEQ ID NO: 225 - protein, 11 aa, synthetic construct
RASQSINSYLD

SEQ ID NO: 226 - protein, 7 aa, synthetic construct
AASSLQS

SEQ ID NO: 227 - protein, 9 aa, synthetic construct
QQYYSTPFT

SEQ ID NO: 228 - protein, 448 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARVGLMGPFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 229 - protein, 214 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 230 - protein, 118 aa, synthetic construct
EVQLVESGGGLVKPGGSLRLSCAASGFTFSSYSMNWVRQAPGKGLEWVSSISSSSSYIYYADSVKGRFTISRDNAKNSLYLQMNSLRAEDTAVYYCARVGLMGPFDIWGQGTMVTVSS

SEQ ID NO: 231 - protein, 107 aa, synthetic construct
EIVLTQSPGTLSLSPGERATLSCRASQSVSRYLGWYQQKPGQAPRLLIYGASTRATGIPDRFSGSGSGTDFTLTITRLEPEDFAVYYCQQYGSSPWTFGQGTKVEIK

SEQ ID NO: 232 - protein, 8 aa, synthetic construct
GFTFSSYS

SEQ ID NO: 233 - protein, 8 aa, synthetic construct
ISSSSSYI

SEQ ID NO: 234 - protein, 11 aa, synthetic construct
ARVGLMGPFDI

SEQ ID NO: 235 - protein, 6 aa, synthetic construct
QSVSRY

SEQ ID NO: 236 - skipped

SEQ ID NO: 237 - protein, 9 aa, synthetic construct
QQYGSSPWT

SEQ ID NO: 238 - protein, 21 aa, synthetic construct
IYIWAPLAGTCGVLLLSLVIT

SEQ ID NO: 239 - protein, 46 aa, synthetic construct
LLPSWAITLISVNGIFVICCLTYCFAPRCRERRRNERLRRESVRPV

SEQ ID NO: 240 - skipped

SEQ ID NO: 241 - protein, 6 aa, synthetic construct
GSGGSN

SEQ ID NO: 242 - protein, 5 aa, synthetic construct
GGGSN

SEQ ID NO: 243 - protein, 6 aa, synthetic construct
GGGGSN

SEQ ID NO: 244 - protein, 7 aa, synthetic construct
GGGGGSN

SEQ ID NO: 245 - protein, 8 aa, synthetic construct
GGGGGGSN

SEQ ID NO: 246 - protein, 27 aa, synthetic construct
SGGGGSGGGGSGGGGSGGGGSGGGSLQ

SEQ ID NO: 247 - protein, 10 aa, synthetic construct
GDVEXINPGP

SEQ ID NO: 248 - protein, 22 aa, synthetic construct
GSGATNFSLLKQAGDVEENPGP

SEQ ID NO: 249 - protein, 23 aa, synthetic construct
GSGQCTNYALLKLAGDVESNPGP

SEQ ID NO: 250 - protein, 21 aa, synthetic construct
GSGEGRGSLLTCGDVEENPGP

SEQ ID NO: 251 - protein, 27 aa, synthetic construct
RAKRSGSGATNFSLLKQAGDVEENPGP

SEQ ID NO: 252 - protein, 18 aa, synthetic construct
MDWTWILFLVAAATRVHS

SEQ ID NO: 253 - protein, 27 aa, synthetic construct
NIKGSPWKGSLLLLLVSNLLLCQSVAP

SEQ ID NO: 254 - protein, 20 aa, synthetic construct
MYRMQLLSCIALSLALVTNS

SEQ ID NO: 255 - protein, 396 aa, synthetic construct
TCGAGGTCGACGGTATCGATAAGCTTGATATCGAATTAGGAGGAAAAACTGTTTCATACAGAAGGCGTCAATTAGGAGGAAAAACTGTTTCATACAGAAGGCGTCAATTAGGAGGAAAAACTGTTTCATACAGAAGGCGTCAATTGGTCCCATCGAATTAGGAGGAAAAACTGTTTCATACAGAAGGCGTCAATTAGGAGGAAAAACTGTTTCATACAGAAGGCGTCAATTAGGAGGAAAAACTGTTTCATACAGAAGGCGTCAATTGGTCCCGGGACATTTTGACACCCCCATAATATTTTTCCAGAATTAACAGTATAAATTGCATCTCTTGTTCAAGAGTTCCCTATCACTCTCTTTAATCACTACTCACAGTAACCTCAACTCCTGGCCACC

SEQ ID NO: 256 - protein, 30 aa, synthetic construct
GGAGGAAAAACTGTTTCATACAGAAGGCGT

SEQ ID NO: 257 - protein, 113 aa, synthetic construct
CATTTTGACACCCCCATAATATTTTTCCAGAATTAACAGTATAAATTGCATCTCTTGTTCAAGAGTTCCCTATCACTCTCTTTAATCACTACTCACAGTAACCTCAACTCCTG

SEQ ID NO: 258 - protein, 114 aa, synthetic construct
NWVNVISDLKKIEDLIQSMHIDATLYTESDVHPSCKVTAMKCFLLELQVISLESGDASIHDTVENLIILANDSLSSNGNVTESGCKECEELEEKNIKEFLQSFVHIVQMFINTS

SEQ ID NO: 259 - protein, 297 aa, synthetic construct
ITCPPPMSVEHADIWVKSYSLYSRERYICNSGFKRKAGTSSLTECVLNKATNVAHWTTPSLKCIREPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 65 aa, synthetic construct
ITCPPPMSVEHADIWVKSYSLYSRERYICNSGFKRKAGTSSLTECVLNKATNVAHWTTPSLKCIR

SEQ ID NO: 261 - protein, 135 aa, synthetic construct
MVLGTIDLCSCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 262 - protein, 162 aa, synthetic construct
MRISKPHLRSISIQCYLCLLLNSHFLTEAGIHVFILGCFSAGLPKTEANWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 263 - protein, 114 aa, synthetic construct
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 264 - protein, 92 aa, synthetic construct
ITCPPPMSVEHADIWVKSYSLYSRERYICNSGFKRKAGTSSLTECVLNKATNVAHWTTPSLKCIRDPALVHQRPAPPSTVTTAGVSEQIDNO

SEQ ID NO: 265 - protein, 182 aa, synthetic construct
ITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTST

SEQ ID NO: 266 - protein, 267 aa, synthetic construct
MAPRRARGCRTLGLPALLLLLLLRPPATRGITCPPPMSVEHADIWVKSYSLYSRERYICNSGFKRKAGTSSLTECVLNKATNVAHWTTPSLKCIRDPALVHQRPAPPSTVTTAGVTPQPESLSPSGKEPAASSPSSNNTAATTAAIVPGSQLMPSKSPSTGTTEISSHESSHGTPSQTTAKNWELTASASHQPPGVYPQGHSDTTVAISTSTVLLCGLSAVSLLACYLKSRQTPPLASVEMEAMEALPVTWGTSSRDEDLENCSHHL

SEQ ID NO: 267 - protein, 197 aa, synthetic construct
RNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNAS

SEQ ID NO: 268 - protein, 306 aa, synthetic construct
IWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCS

SEQ ID NO: 269 - protein, 157 aa, synthetic construct
YFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNED

SEQ ID NO: 270 - protein, 156 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARVPGHGRKTQFESSSYEGYFLACEKERDLFKLILKKEDELGDRSIMFTVQNED

SEQ ID NO: 271 - protein, 133 aa, synthetic construct [sequence omitted]

SEQ ID NO: 272 - protein, 153 aa, synthetic construct; identical to SEQ ID NO: 13

SEQ ID NO: 273 - protein, 261 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 274 - protein, 120 aa, synthetic construct
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSS

SEQ ID NO: 275 - protein, 108 aa, synthetic construct
DIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIK

SEQ ID NO: 276 - protein, 248 aa, synthetic construct
QVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSGGGGSGGGGSGGGGSGGGGSDIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIK

SEQ ID NO: 277 - protein, 112 aa, synthetic construct
QLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSS

SEQ ID NO: 278 - protein, 112 aa, synthetic construct [sequence omitted]

SEQ ID NO: 279 - protein, 244 aa, synthetic construct
QLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSGGGGSGGGGSGGGGSGGGGSDIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIK

SEQ ID NO: 280 - protein, 120 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSS

SEQ ID NO: 281 - protein, 112 aa, synthetic construct
DIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 282 - protein, 252 aa, synthetic construct
QVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSGGGGSGGGGSGGGGSGGGGSDIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIR

SEQ ID NO: 283 - protein, 126 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSS

SEQ ID NO: 284 - protein, 107 aa, synthetic construct
DIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 285 - protein, 253 aa, synthetic construct
QVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIK

SEQ ID NO: 286 - DNA, 49 bp, synthetic construct
  source 1..49: mol_type = other DNA
ttctccccagccctgctcgtggtgaccgaaggggacaacgccaccttca

SEQ ID NO: 287 - DNA, 49 bp, synthetic construct
  source 1..49: mol_type = other DNA
tacctctgtggggccatctccctggcccccaaggcgcagatcaaagaga

SEQ ID NO: 288 - protein, 530 aa, synthetic construct
LTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALE

SEQ ID NO: 289 - protein, 529 aa, synthetic construct
LTPQQVVAIASNNGGKQALETVQRLLPVLCQAHGLTPEQVVAIASNIGGKQALETVQALLPVLCQAHGLTPEQVVAIASNIGGKQALETVQALLPVLCQAHGLTPQQVVAIASNNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGRPALE

SEQ ID NO: 290 - DNA, 2814 bp, synthetic construct
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 291 - DNA, 2829 bp, synthetic construct
  source 1..2829: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccccagcaagtcgtcgcaatcgccagcaataacggagggaagcaagccctcgaaaccgtgcagcggttgcttcctgtgctctgccaggcccacggccttacccctgagcaggtggtggccatcgcaagtaacattggaggaaagcaagccttggagacagtgcaggccctgttgcccgtgctgtgccaggcacacggcctcacaccagagcaggtcgtggccattgcctccaacatcggggggaaacaggctctggagaccgtccaggccctgctgcccgtcctctgtcaagctcacggcctgactccccaacaagtggtcgccatcgcctctaataacggcgggaagcaggcactggaaacagtgcagagactgctccctgtgctttgccaagctcatgggttgaccccccaacaggtcgtcgctattgcctcaaacaacgggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgaccccccagcaggtggtggctatcgccagcaataatgggggcaagcaggccctggaaacagtccagcgcctgctgccagtgctttgccaggctcacgggctcactcccgaacaggtcgtggcaatcgcctccaacggagggaagcaggctctggagaccgtgcagagactgctgcccgtcttgtgccaggcccacggactcacacctcagcaggtcgtcgccattgcctctaacaacgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacggcggaggaaagcaggctctcgaaactgtgcagcggctgcttcctgtgctgtgtcaggctcatgggctgaccccccagcaagtggtggctattgcctctaacaatggaggcaagcaagcccttgagacagtccagaggctgttgccagtgctgtgccaggcccacgggctcacaccccagcaggtggtcgccatcgccagtaacggcgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 292 - protein, 530 aa, synthetic construct
LTPEQVVAIASNIGGKQALETVQALLPVLCQAHGLTPEQVVAIASHDGGKQALETVQRLLPVLCQAHGLTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNGGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPQQVVAIASNNGGKQALETVQRLLPVLCQAHGLTPEQVVAIASHDGGKQALETVQRLLPVLCQAHGLTPEQVVAIASHDGGKQALETVQRLLPVLCQAHGLTPEQVVAIASNIGGKQALETVQALLPVLCQAHGLTPQQVVAIASNGGGRPALE

SEQ ID NO: 293 - protein, 529 aa, synthetic construct
LTPEQVVAIASHDGGKQALETVQRLLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNIGGKQALETVQALLPVLCQAHGLTPQQVVAIASNGGGKQALETVQRLLPVLCQAHGLTPEQVVAIASHDGGKQALETVQRLLPVLCQAHGLTPQQVVAIASNGGGKQALETVQRLLPVLCQAHGLTPQQVVAIASNNGGKQALETVQRLLPVLCQAHGLTPEQVVAIASNGGKQALETVQRLLPVLCQAHGLTPQQVVAIASNNGGKQALETVQRLLPVLCQAHGLTPEQVVAIASHDGGKQALETVQRLLPVLCQAHGLTPQQVVAIASNGGGRPALE

SEQ ID NO: 294 - DNA, 2814 bp, synthetic construct
  source 1..2814: mol_type = other DNA
atgggcgatcctaaaaagaaacgtaaggtcatcgattacccatacgatgttccagattacgctatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgaccccggagcaggtggtggccatcgccagcaatattggtggcaagcaggcgctggagacggtgcaggcgctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaatggcggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccccagcaggtggtggccatcgccagcaataatggtggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagccacgatggcggcaagcaggcgctggagacggtccagcggctgttgccggtgctgtgccaggcccacggcttgaccccggagcaggtggtggccatcgccagcaatattggtggcaagcaggcgctggagacggtgcaggcgctgttgccggtgctgtgccaggcccacggcttgacccctcagcaggtggtggccatcgccagcaatggcggcggcaggccggcgctggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 295 - DNA, 2829 bp, synthetic construct
  source 1..2829: mol_type = other DNA
atgggcgatcctaaaaagaaacgtaaggtcatcgataaggagaccgccgctgccaagttcgagagacagcacatggacagcatcgatatcgccgatctacgcacgctcggctacagccagcagcaacaggagaagatcaaaccgaaggttcgttcgacagtggcgcagcaccacgaggcactggtcggccacgggtttacacacgcgcacatcgttgcgttaagccaacacccggcagcgttagggaccgtcgctgtcaagtatcaggacatgatcgcagcgttgccagaggcgacacacgaagcgatcgttggcgtcggcaaacagtggtccggcgcacgcgctctggaggccttgctcacggtggcgggagagttgagaggtccaccgttacagttggacacaggccaacttctcaagattgcaaaacgtggcggcgtgaccgcagtggaggcagtgcatgcatggcgcaatgcactgacgggtgccccgctcaacttgacccccgagcaagtcgtcgcaatcgccagccatgatggagggaagcaagccctcgaaaccgtgcagcggttgcttcctgtgctctgccaggcccacggccttacccctcagcaggtggtggccatcgcaagtaacggaggaggaaagcaagccttggagacagtgcagcgcctgttgcccgtgctgtgccaggcacacggcctcacaccagagcaggtcgtggccattgcctcccatgacggggggaaacaggctctggagaccgtccagaggctgctgcccgtcctctgtcaagctcacggcctgactccccaacaagtggtcgccatcgcctctaatggcggcgggaagcaggcactggaaacagtgcagagactgctccctgtgctttgccaagctcatgggttgaccccccaacaggtcgtcgctattgcctcaaacggggggggcaagcaggcccttgagactgtgcagaggctgttgccagtgctgtgtcaggctcacgggctcactccacaacaggtggtcgcaattgccagcaacggcggcggaaagcaagctcttgaaaccgtgcaacgcctcctgcccgtgctctgtcaggctcatggcctgacaccacaacaagtcgtggccatcgccagtaataatggcgggaaacaggctcttgagaccgtccagaggctgctcccagtgctctgccaggcacacgggctgacccccgagcaggtggtggctatcgccagcaatattgggggcaagcaggccctggaaacagtccaggccctgctgccagtgctttgccaggctcacgggctcactccccagcaggtcgtggcaatcgcctccaacggcggagggaagcaggctctggagaccgtgcagagactgctgcccgtcttgtgccaggcccacggactcacacctgaacaggtcgtcgccattgcctctcacgatgggggcaaacaagccctggagacagtgcagcggctgttgcctgtgttgtgccaagcccacggcttgactcctcaacaagtggtcgccatcgcctcaaatggcggcggaaaacaagctctggagacagtgcagaggttgctgcccgtcctctgccaagcccacggcctgactccccaacaggtcgtcgccattgccagcaacaacggaggaaagcaggctctcgaaactgtgcagcggctgcttcctgtgctgtgtcaggctcatgggctgacccccgagcaagtggtggctattgcctctaatggaggcaagcaagcccttgagacagtccagaggctgttgccagtgctgtgccaggcccacgggctcacaccccagcaggtggtcgccatcgccagtaacaacgggggcaaacaggcattggaaaccgtccagcgcctgcttccagtgctctgccaggcacacggactgacacccgaacaggtggtggccattgcatcccatgatgggggcaagcaggccctggagaccgtgcagagactcctgccagtgttgtgccaagctcacggcctcacccctcagcaagtcgtggccatcgcctcaaacggggggggccggcctgcactggagagcattgttgcccagttatctcgccctgatccggcgttggccgcgttgaccaacgaccacctcgtcgccttggcctgcctcggcgggcgtcctgcgctggatgcagtgaaaaagggattgggggatcctatcagccgttcccagctggtgaagtccgagctggaggagaagaaatccgagttgaggcacaagctgaagtacgtgccccacgagtacatcgagctgatcgagatcgcccggaacagcacccaggaccgtatcctggagatgaaggtgatggagttcttcatgaaggtgtacggctacaggggcaagcacctgggcggctccaggaagcccgacggcgccatctacaccgtgggctcccccatcgactacggcgtgatcgtggacaccaaggcctactccggcggctacaacctgcccatcggccaggccgacgaaatgcagaggtacgtggaggagaaccagaccaggaacaagcacatcaaccccaacgagtggtggaaggtgtacccctccagcgtgaccgagttcaagttcctgttcgtgtccggccacttcaagggcaactacaaggcccagctgaccaggctgaaccacatcaccaactgcaacggcgccgtgctgtccgtggaggagctcctgatcggcggcgagatgatcaaggccggcaccctgaccctggaggaggtgaggaggaagttcaacaacggcgagatcaacttcgcggccgactgataa

SEQ ID NO: 296 - DNA, 615 bp, synthetic construct
  source 1..615: mol_type = other DNA
atggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagaaaagaacatcaaagagtttctgcagagcttcgtccacatcgtgcagatgttcatcaacacctcatcaggtggcggtggaagcggaggtggcggtagtggcggcggaggctcaggcggcggaggttccggcggaggatctcttcagctcctgccatcttgggctatcaccctgattagtgtgaatgggatctttgtcatctgttgtctcacgtactgtttcgctccccggtgcagagagagaaggcgcaacgaaagactgcggagagaaagcgtcagacccgtg

SEQ ID NO: 297 - DNA, 735 bp, synthetic construct
  source 1..735: mol_type = other DNA
atggattggacctggattctgttcctggtggccgctgccacaagagtgcatagccagggccaagaccggcacatgatccggatgagacagctgatcgacatcgtggaccagctgaagaactacgtgaacgacctggtgcctgagttcctgcctgctcctgaggacgtggaaacaaattgcgagtggtccgccttcagctgcttccagaaggcccagctgaaaagcgccaacaccggcaacaacgagcggatcatcaacgtgtccatcaagaagctgaagcggaagcctcctagcaccaatgccggaagaaggcagaagcacagactgacctgtcctagctgcgacagctacgagaagaagcctccaaaagagttcctggaacggttcaagagcctgctgcagaagatgatccaccagcacctgagcagcagaacccacggctctgaagattctagcggaggcggaggaagtggtggcggaggttctggtggcggtggatcaggcggtggcggatctggcggcggaggcagtggcggaggtggaagcggtggtggtggctctggcggaggcggtagcggcggaggcggatctcttcaattgctgcctagctgggccatcacactgatctccgtgaacggcatcttcgtgatctgctgcctgacctactgcttcgcccctagatgcagagagcggagaagaaacgagcggctgagaagagaatctgtgcggcctgtt

SEQ ID NO: 298 - DNA, 1431 bp, synthetic construct
  source 1..1431: mol_type = other DNA
atggattggacctggattctgttcctggtggccgctgccacaagagtgcatagccagggccaagaccggcacatgatccggatgagacagctgatcgacatcgtggaccagctgaagaactacgtgaacgacctggtgcctgagttcctgcctgctcctgaggacgtggaaacaaattgcgagtggtccgccttcagctgcttccagaaggcccagctgaaaagcgccaacaccggcaacaacgagcggatcatcaacgtgtccatcaagaagctgaagcggaagcctcctagcaccaatgccggaagaaggcagaagcacagactgacctgtcctagctgcgacagctacgagaagaagcctccaaaagagttcctggaacggttcaagagcctgctgcagaagatgatccaccagcacctgagcagcagaacccacggctctgaagattctagcggaggcggaggaagtggtggcggaggttctggtggcggtggatcaggcggtggcggatctggcggcggaggcagtggcggaggtggaagcggtggtggtggctctggcggaggcggtagcggcggaggcggatctcttcaattgctgcctagctgggccatcacactgatctccgtgaacggcatcttcgtgatctgctgcctgacctactgcttcgcccctagatgcagagagcggagaagaaacgagcggctgagaagagaatctgtgcggcctgttagagccaagagatctggaagcggcgccaccaactttagcctgctgaaacaggctggcgacgtggaagagaaccctggacctatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagaaaagaacatcaaagagtttctgcagagcttcgtccacatcgtgcagatgttcatcaacacctcatcaggtggcggtggaagcggaggtggcggtagtggcggcggaggctcaggcggcggaggttccggcggaggatctcttcagctcctgccatcttgggctatcaccctgattagtgtgaatgggatctttgtcatctgttgtctcacgtactgtttcgctccccggtgcagagagagaaggcgcaacgaaagactgcggagagaaagcgtcagacccgtg

SEQ ID NO: 299 - DNA, 2202 bp, synthetic construct
  source 1..2202: mol_type = other DNA
tcgaggtcgacggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctggccaccatggattggacctggattctgttcctcgtggccgccgctacccgcgtgcactccatctgggagctgaagaaagacgtgtacgtggtggaattggattggtaccccgacgccccaggcgagatggtggttctgacttgcgacacgccggaggaggacggcatcacctggacgctggaccagagcagcgaagtgctgggctctggtaagaccttaaccatccaggtcaaggagttcggtgatgcaggacagtacacgtgtcacaagggcggggaggtcttgtcccatagcctgctgctcctgcacaagaaggaagacggcatctggtctacggatatccttaaagaccagaaggagcccaagaacaaaaccttcctgcgatgcgaggctaagaattactccgggcgctttacctgttggtggctcacaaccatctcgaccgacctgaccttctccgtgaagtcttccaggggatcttctgatccgcaaggcgttacctgcggggccgctaccctgtcggctgagcgcgtccgtggtgacaacaaggagtacgagtactcggtggaatgccaggaggacagcgcctgcccagcggccgaagagtccttgcccatcgaggtgatggtggacgctgtacacaagctcaagtacgagaactacacctcctccttctttatacgcgacatcatcaagcctgacccccctaaaaacctgcagctgaagccccttaaaaactctcgacaggtcgaggtatcctgggagtaccccgacacctggtctactcctcattcgtatttcagcctgaccttctgcgtgcaggtgcagggcaagtccaagcgcgagaagaaggaccgcgtgttcaccgacaaaaccagcgcgactgtcatttgtcggaagaacgcgagcatctccgtacgggcacaggatcgctattactccagcagttggtcagagtgggcttcggttccgtgttctggaggaggtggcggcggctcacgcaacctgcccgtggcgacgccggacccgggtatgtttccttgtctgcaccactctcagaacttgctccgtgccgtctcgaatatgctgcagaaggcccgccagactctggagttctatccatgcactagtgaggagatcgatcatgaggacatcaccaaggacaagacatccaccgtggaggcttgtctgccgctggagttgacaaagaacgagagctgcctcaatagccgcgagacttccttcatcaccaacggctcctgcctcgcgagccgtaagacctcctttatgatggcgctgtgcctgtcaagtatctacgaagatctgaaaatgtatcaggtggagttcaaaactatgaacgcaaaactcctgatggatcccaagcgccaaattttcttggaccagaacatgcttgccgtgatcgacgagctgatgcaggccctgaacttcaactccgagaccgtccctcagaagtcgtctctggaggaacccgacttttacaagacaaagatcaagctgtgcatcctgctacacgcgttccgcattcgggccgtgaccattgaccgcgtgatgtcctacctgaatgcctcgtccggcggggggggctccggtgggggcggctctggcggaggcggctcaggtggcggcggctccggcggaggttcgcttcagcttctgcccagttgggcaatcactctgatttccgtcaacggaatcttcgtcatctgctgcctgacctactgctttgctcccagatgtagagagcgccgccgcaacgagcgcctgcgtcgtgagagcgtgaggcctgtgtga

SEQ ID NO: 300 - DNA, 1014 bp, synthetic construct
  source 1..1014: mol_type = other DNA
tcgaggtcgacggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctggccaccatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagaaaagaacatcaaagagtttctgcagagcttcgtccacatcgtgcagatgttcatcaacacctcatcaggtggcggtggaagcggaggtggcggtagtggcggcggaggctcaggcggcggaggttccggcggaggatctcttcagctcctgccatcttgggctatcaccctgattagtgtgaatgggatctttgtcatctgttgtctcacgtactgtttcgctccccggtgcagagagagaaggcgcaacgaaagactgcggagagaaagcgtcagacccgtgtga

SEQ ID NO: 301 - DNA, 1134 bp, synthetic construct
  source 1..1134: mol_type = other DNA
tcgaggtcgacggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctggccaccatggattggacctggattctgttcctggtggccgctgccacaagagtgcatagccagggccaagaccggcacatgatccggatgagacagctgatcgacatcgtggaccagctgaagaactacgtgaacgacctggtgcctgagttcctgcctgctcctgaggacgtggaaacaaattgcgagtggtccgccttcagctgcttccagaaggcccagctgaaaagcgccaacaccggcaacaacgagcggatcatcaacgtgtccatcaagaagctgaagcggaagcctcctagcaccaatgccggaagaaggcagaagcacagactgacctgtcctagctgcgacagctacgagaagaagcctccaaaagagttcctggaacggttcaagagcctgctgcagaagatgatccaccagcacctgagcagcagaacccacggctctgaagattctagcggaggcggaggaagtggtggcggaggttctggtggcggtggatcaggcggtggcggatctggcggcggaggcagtggcggaggtggaagcggtggtggtggctctggcggaggcggtagcggcggaggcggatctcttcaattgctgcctagctgggccatcacactgatctccgtgaacggcatcttcgtgatctgctgcctgacctactgcttcgcccctagatgcagagagcggagaagaaacgagcggctgagaagagaatctgtgcggcctgtttaa

SEQ ID NO: 302 - DNA, 1830 bp, synthetic construct
  source 1..1830: mol_type = other DNA
tcgaggtcgacggtatcgataagcttgatatcgaattaggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccgggacattttgacacccccataatatttttccagaattaacagtataaattgcatctcttgttcaagagttccctatcactctctttaatcactactcacagtaacctcaactcctggccaccatggattggacctggattctgttcctggtggccgctgccacaagagtgcatagccagggccaagaccggcacatgatccggatgagacagctgatcgacatcgtggaccagctgaagaactacgtgaacgacctggtgcctgagttcctgcctgctcctgaggacgtggaaacaaattgcgagtggtccgccttcagctgcttccagaaggcccagctgaaaagcgccaacaccggcaacaacgagcggatcatcaacgtgtccatcaagaagctgaagcggaagcctcctagcaccaatgccggaagaaggcagaagcacagactgacctgtcctagctgcgacagctacgagaagaagcctccaaaagagttcctggaacggttcaagagcctgctgcagaagatgatccaccagcacctgagcagcagaacccacggctctgaagattctagcggaggcggaggaagtggtggcggaggttctggtggcggtggatcaggcggtggcggatctggcggcggaggcagtggcggaggtggaagcggtggtggtggctctggcggaggcggtagcggcggaggcggatctcttcaattgctgcctagctgggccatcacactgatctccgtgaacggcatcttcgtgatctgctgcctgacctactgcttcgcccctagatgcagagagcggagaagaaacgagcggctgagaagagaatctgtgcggcctgttagagccaagagatctggaagcggcgccaccaactttagcctgctgaaacaggctggcgacgtggaagagaaccctggacctatggactggacttggatactctttctggtcgctgccgccacacgggtgcactctaattgggtcaacgtgatcagcgacctgaagaagatcgaggacctgatccagagcatgcacatcgacgccacactgtacaccgagtccgatgtgcaccctagctgcaaagtgaccgccatgaagtgctttctgctggaactgcaagtgatcagcctggaaagcggcgacgccagcatccacgataccgtggaaaatctgatcatcctggccaacaacagcctgtccagcaacggcaatgtgaccgagagcggctgcaaagagtgcgaggaactggaagaaaagaacatcaaagagtttctgcagagcttcgtccacatcgtgcagatgttcatcaacacctcatcaggtggcggtggaagcggaggtggcggtagtggcggcggaggctcaggcggcggaggttccggcggaggatctcttcagctcctgccatcttgggctatcaccctgattagtgtgaatgggatctttgtcatctgttgtctcacgtactgtttcgctccccggtgcagagagagaaggcgcaacgaaagactgcggagagaaagcgtcagacccgtgtaa

SEQ ID NO: 303 - protein, 601 aa, synthetic construct
MDWTWILFLVAAATRVHSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGGGSGGGGSGGGGSGGGGSGGGSLQLLPSWAITLISVNGIFVICCLTYCFAPRCRERRRNERLRRESVRPV

SEQ ID NO: 304 - DNA, 1803 bp, synthetic construct
  source 1..1803: mol_type = other DNA
atggattggacctggattctgttcctcgtggccgccgctacccgcgtgcactccatctgggagctgaagaaagacgtgtacgtggtggaattggattggtaccccgacgccccaggcgagatggtggttctgacttgcgacacgccggaggaggacggcatcacctggacgctggaccagagcagcgaagtgctgggctctggtaagaccttaaccatccaggtcaaggagttcggtgatgcaggacagtacacgtgtcacaagggcggggaggtcttgtcccatagcctgctgctcctgcacaagaaggaagacggcatctggtctacggatatccttaaagaccagaaggagcccaagaacaaaaccttcctgcgatgcgaggctaagaattactccgggcgctttacctgttggtggctcacaaccatctcgaccgacctgaccttctccgtgaagtcttccaggggatcttctgatccgcaaggcgttacctgcggggccgctaccctgtcggctgagcgcgtccgtggtgacaacaaggagtacgagtactcggtggaatgccaggaggacagcgcctgcccagcggccgaagagtccttgcccatcgaggtgatggtggacgctgtacacaagctcaagtacgagaactacacctcctccttctttatacgcgacatcatcaagcctgacccccctaaaaacctgcagctgaagccccttaaaaactctcgacaggtcgaggtatcctgggagtaccccgacacctggtctactcctcattcgtatttcagcctgaccttctgcgtgcaggtgcagggcaagtccaagcgcgagaagaaggaccgcgtgttcaccgacaaaaccagcgcgactgtcatttgtcggaagaacgcgagcatctccgtacgggcacaggatcgctattactccagcagttggtcagagtgggcttcggttccgtgttctggaggaggtggcggcggctcacgcaacctgcccgtggcgacgccggacccgggtatgtttccttgtctgcaccactctcagaacttgctccgtgccgtctcgaatatgctgcagaaggcccgccagactctggagttctatccatgcactagtgaggagatcgatcatgaggacatcaccaaggacaagacatccaccgtggaggcttgtctgccgctggagttgacaaagaacgagagctgcctcaatagccgcgagacttccttcatcaccaacggctcctgcctcgcgagccgtaagacctcctttatgatggcgctgtgcctgtcaagtatctacgaagatctgaaaatgtatcaggtggagttcaaaactatgaacgcaaaactcctgatggatcccaagcgccaaattttcttggaccagaacatgcttgccgtgatcgacgagctgatgcaggccctgaacttcaactccgagaccgtccctcagaagtcgtctctggaggaacccgacttttacaagacaaagatcaagctgtgcatcctgctacacgcgttccgcattcgggccgtgaccattgaccgcgtgatgtcctacctgaatgcctcgtccggcggggggggctccggtgggggcggctctggcggaggcggctcaggtggcggcggctccggcggaggttcgcttcagcttctgcccagttgggcaatcactctgatttccgtcaacggaatcttcgtcatctgctgcctgacctactgctttgctcccagatgtagagagcgccgccgcaacgagcgcctgcgtcgtgagagcgtgaggcctgtg

SEQ ID NO: 305 - protein, 248 aa, synthetic construct
MDWTWILFLVAAATRVHSYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDSGGGGSGGGGSGGGGSGGGGSGGGSLQLLPSWAITLISVNGIFVICCLTYCFAPRCRERRRNERLRRESVRPV

SEQ ID NO: 306 - DNA, 744 bp, synthetic construct
  source 1..744: mol_type = other DNA
atggattggacctggattttgttcctggtggctgcagccactcgggtccacagctacttcggcaagctggagagtaaactttccgtgatccgcaacctcaacgaccaagtgctgttcattgaccagggcaaccgccccctctttgaagacatgaccgacagcgattgtcgtgacaatgcgccccgcaccatctttataatttccatgtacaaggatagccagccgcggggaatggcggtaacgatctcggtgaagtgtgaaaaaatctcgaccctgtcatgcgagaacaagatcatttccttcaaagagatgaatccacccgacaacatcaaggacaccaagtccgacatcatcttcttccagcgcagcgtgcctgggcatgacaacaagatgcagttcgagagctcgtcttacgagggctatttcctggcctgcgagaaggagcgtgacctgttcaagcttatcctgaagaaggaggacgagctgggggaccgcagcatcatgtttacagtgcagaacgaggattctggtggcggcggtagcgggggcgggggttctggcggcggaggctccggtggaggaggctccggcggcggttcgttgcagctgctgcccagttgggccatcactctaatctccgtcaacggcatcttcgtcatctgttgcctgacctactgctttgctccgaggtgtagagagcgacgccgcaacgagcgcctccgccgtgagtccgtgcgccctgtt

SEQ ID NO: 307 - protein, 247 aa, synthetic construct
MDWTWILFLVAAATRVHSYFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARVPGHGRKTQFESSSYEGYFLACEKERDLFKLILKKEDELGDRSIMFTVQNEDSGGGGSGGGGSGGGGSGGGGSGGGSLQLLPSWAITLISVNGIFVICCLTYCFAPRCRERRRNERLRRESVRPV

SEQ ID NO: 308 - DNA, 741 bp, synthetic construct
  source 1..741: mol_type = other DNA
atggattggacctggatcctgttcctggtggcggcggcaactcgcgtacatagctatttcggcaagctggagagcaaactatcagtgatccgcaacctcaacgaccaggtcttgtttattgaccagggcaaccgccccctgttcgaagacatgacagacagcgattgtcgcgacaatgctcctcgcaccatctttatcatctccaagtactccgattctcgagccagaggtctcgccgtgaccatctccgtgaagtgcgagaagatatccactctttcatgcgagaacaagatcatttccttcaaggagatgaatccacccgacaacatcaaggacaccaagtccgacatcatcttcttcgctcgtgtgcctggccacggccgcaagacccagttcgagagttccagctacgagggctacttcctggcctgtgagaaagagcgggaccttttcaaattgattctgaagaaggaggacgagctgggcgaccgtagcatcatgtttacggtgcagaacgaggattctggcgggggcggttctggagggggtgggtcgggcggtggtggctccgggggaggcggctccggaggaggctcgctgcagctgctgcccagttgggccatcacgctcatttctgtcaacggcatcttcgtcatctgttgcctgacctactgctttgctccgaggtgtagggaacgtcgtcgcaacgagcgcctgcgccgcgagtctgttcggccggtg

SEQ ID NO: 309 - protein, 833 aa, synthetic construct
MDWTWILFLVAAATRVHSIWELKKDVYVVELDWYPDAPGEMVVLTCDTPEEDGITWTLDQSSEVLGSGKTLTIQVKEFGDAGQYTCHKGGEVLSHSLLLLHKKEDGIWSTDILKDQKEPKNKTFLRCEAKNYSGRFTCWWLTTISTDLTFSVKSSRGSSDPQGVTCGAATLSAERVRGDNKEYEYSVECQEDSACPAAEESLPIEVMVDAVHKLKYENYTSSFFIRDIIKPDPPKNLQLKPLKNSRQVEVSWEYPDTWSTPHSYFSLTFCVQVQGKSKREKKDRVFTDKTSATVICRKNASISVRAQDRYYSSSWSEWASVPCSGGGGGGSRNLPVATPDPGMFPCLHHSQNLLRAVSNMLQKARQTLEFYPCTSEEIDHEDITKDKTSTVEACLPLELTKNESCLNSRETSFITNGSCLASRKTSFMMALCLSSIYEDLKMYQVEFKTMNAKLLMDPKRQIFLDQNMLAVIDELMQALNFNSETVPQKSSLEEPDFYKTKIKLCILLHAFRIRAVTIDRVMSYLNASSGGGGSGGGGSGGGGSGGGGSGGGSLQLLPSWAITLISVNGIFVICCLTYCFAPRCRERRRNERLRRESVRPVRAKRSGSGATNFSLLKQAGDVEENPGPMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGGGGSGGGGSGGGSLQLLPSWAITLISVNGIFVICCLTYCFAPRCRERRRNERLRRESVRPV

SEQ ID NO: 310 - DNA, 2499 bp, synthetic construct
  source 1..2499: mol_type = other DNA
atggattggacctggattctgtttttggtggcggccgccacccgcgtccattccatatgggaattaaagaaggacgtgtacgtggtggagcttgactggtaccctgacgctcccggcgagatggtggtcctgacctgcgatacccccgaggaggacggcatcacctggaccctggaccagagctccgaggtgctgggaagcggtaaaaccctcaccatacaggtgaaggagttcggtgatgctggccagtacacctgtcacaaaggcggggaggtactttcccattcgctgctcctgcttcacaagaaggaagacggcatttggtcgaccgatattctgaaggaccagaaggaaccaaaaaacaagacctttttgcgttgcgaggccaagaactattccggccgcttcacctgctggtggctaacgaccatctcaaccgacctgaccttctccgttaagtctagccgtggtagctccgaccctcagggggtaacctgcggggctgcgacgctgagtgcggagcgggtacgcggcgacaacaaggagtacgagtactccgtggaatgtcaggaggatagtgcctgcccagcggccgaggagtccctgcccatcgaggtaatggtggacgcggtccacaagctgaagtacgagaactacactagctccttctttattagagacatcatcaagcccgacccgcctaagaacctgcagctgaaacctctcaaaaactcccgccaggttgaagtgtcgtgggagtaccctgacacctggtctacaccgcacagctatttctcgcttacattctgcgtgcaggtgcagggcaaatccaagcgcgagaagaaagaccgcgtgttcaccgacaaaacctctgctacagtgatctgccgaaagaatgcttctatctccgtgcgtgctcaggaccgctactactcatcaagctggagtgagtgggcctccgtgccatgttccggggggggcggcggtggctcccgcaacctgcccgttgcaacgcccgacccgggtatgttcccatgcctgcaccactcccagaatctgttacgtgctgtaagcaatatgcttcagaaggctcgccagactctggagttctacccctgcacttcggaggagatcgaccacgaagacatcaccaaggacaagacatccaccgtggaggcgtgcctccctttggagctcacaaagaacgagtcctgtctaaactcaagggagactagcttcatcactaatggatcgtgtctggcgtcgcgcaagacctctttcatgatggccctgtgcctgtccagcatctatgaggaccttaaaatgtatcaggtcgagttcaagactatgaacgcaaagctgctgatggatcccaagcggcaaattttcctggaccagaacatgctggcggtgatcgacgagctgatgcaggccctcaacttcaacagcgaaactgttcctcagaagtcctccctggaggagccggacttttacaagaccaagatcaagctatgtattttgttacacgccttccgcatccgcgctgtcactattgaccgcgtgatgtcttacctgaacgcatctagtgggggcggaggttccgggggaggcggctctggcggtggcggctctggcggtggtggttcaggaggcgggtcccttcagctgctcccgtcgtgggccatcacactgatctctgtgaacggcatcttcgtcatctgttgtcttacttactgctttgcccccaggtgtagggagagacgccgcaatgaacggctgaggcgtgagagcgtgcgcccggtgagggctaagcgcagcggctccggcgcgaccaacttttcgctgctgaagcaggccggggacgtggaggagaatccaggcccgatggattggacctggatcctgttcttggtggccgcggctactcgggtgcactctaattgggtcaacgtgatctccgacttgaaaaagatcgaggatctgatccagagcatgcatatcgacgccacgctgtacaccgagagcgatgtgcacccctcttgcaaggtgaccgccatgaagtgctttctgctggagctgcaggtcatctctcttgagtcgggcgatgcatctattcatgacaccgtcgagaacctaatcatcctggccaacaactcactgtcttcaaacggcaacgtgacggagtctgggtgcaaggaatgtgaggaattggaggagaaaaacatcaaggagttcctgcagtccttcgtccacatcgtgcagatgtttattaacacgagtagtggcggaggtggcagcggaggcggcggctccggcggtggaggaagtggaggcggcggctctggtggcggttccctacaattgcttccctcttgggctatcaccctcatcagcgtcaacggtatcttcgtcatctgttgtctgacctactgctttgcccctcgatgccgagagcgccgtcgcaacgagcgcctgcgccgggagtccgtgcgccccgtt

SEQ ID NO: 311 - protein, 480 aa, synthetic construct
MDWTWILFLVAAATRVHSYFGKLESKLSVIRNLNDQVLFIDQGNRPLFEDMTDSDCRDNAPRTIFIISMYKDSQPRGMAVTISVKCEKISTLSCENKIISFKEMNPPDNIKDTKSDIIFFQRSVPGHDNKMQFESSSYEGYFLACEKERDLFKLILKKEDELGDRSIMFTVQNEDSGGGGSGGGGSGGGGSGGGGSGGGSLQLLPSWAITLISVNGIFVICCLTYCFAPRCRERRRNERLRRESVRPVRAKRSGSGATNFSLLKQAGDVEENPGPMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGGGGSGGGGSGGGSLQLLPSWAITLISVNGIFVICCLTYCFAPRCRERRRNERLRRESVRPV

SEQ ID NO: 312 - DNA, 1440 bp, synthetic construct
  source 1..1440: mol_type = other DNA
atggattggacctggatccttttcttggtggcggctgcaacccgggttcatagctatttcggcaagctggagtccaagctctccgtgatccgcaacctcaacgaccaggtgctgttcatcgaccagggcaaccgccccctgtttgaggacatgacagattctgattgccgcgacaacgcgccgcgcaccatcttcattatctctatgtacaaggactcccagccacgtggtatggccgtcacgatcagcgtgaagtgcgagaagatttctaccctgtcctgcgagaacaagatcatctcgttcaaggagatgaaccctcccgacaatatcaaggacaccaagtccgacatcatcttcttccagcgatcagtgccaggccacgacaacaagatgcagttcgagagcagcagctacgagggctacttccttgcgtgcgagaaagagcgcgatctgttcaaactgatcctcaagaaggaagacgagctcggggaccgcagcatcatgtttaccgtacagaacgaggacagcggcggcggcggctccggtggaggcggtagtggcggaggcggctcaggcggcggcggctccggaggtgggtcgttgcagctcttgccgagttgggctatcactcttatctccgtaaacggcatcttcgtgatatgctgcctgacgtactgttttgctcctaggtgtcgcgagcgccgccggaacgagagactgcgacgggaatccgtgcgcccggtccgcgcgaagcgtagtggctccggtgctaccaacttttctctgctgaagcaggccggcgacgtggaggagaatcccggccctatggactggacatggatcctgttcctggtggctgcggccacccgcgtccactcaaattgggtcaatgtgatttctgacctgaagaaaattgaggatctcatccagtccatgcacattgacgccactctgtacaccgagagcgatgttcacccctcttgtaaggtgaccgccatgaaatgtttcctgttggagctgcaggtcatttcgctcgagtctggcgacgcatcgatccatgacaccgtggagaacctgattatcctggccaacaactccctttcttcaaatggcaacgtgactgaaagcgggtgcaaagagtgtgaggagctggaagaaaagaacatcaaagagttcctgcaatctttcgtccacattgtgcagatgtttattaacacgtcctcgggtggcggggggtcgggcgggggtggttccggcggaggcgggtccggaggagggggctctggtgggggatccctacagctactgccctcttgggccatcacccttatctccgtcaacggtatcttcgtgatctgttgcctgacttactgctttgccccgagatgtcgtgagcgtaggaggaacgagcgcctgcgccgcgagagcgtgcgtcccgtg

SEQ ID NO: 313 - protein, 339 aa, synthetic construct
MDWTWILFLVAAATRVHSQVQLVESGGGVVQPGRSLRLSCAASGFSFSSTYVCWVRQAPGKGLEWIACIYTGDGTNYSASWAKGRFTISKDSSKNTVYLQMNSLRAEDTAVYFCARPDITYGFAINFWGPGTLVTVSSGGGGSGGGGSGGGGSGGGGSDIQMTQSPSSLSASVGDRVTIKCQASQSISSRLAWYQQKPGKPPKLLIYRASTLASGVPSRFSGSGSGTDFTLTISSLQPEDVATYYCQCTGYGISWPIGGGTKVEIKSGGGGSGGGGSGGGGSGGGGSGGGSLQLLPSWAITLISVNGIFVICCLTYCFAPRCRERRRNERLRRESVRPV

SEQ ID NO: 314 - DNA, 1017 bp, synthetic construct
  source 1..1017: mol_type = other DNA
atggattggacctggatcctgttcctggtggcggccgctacacgagtccacagccaggtccagctcgtggagagcggaggcggcgtggtgcagccaggccgctcgctgcgcctgtcatgcgcagcgtccggtttctccttctcgtcgacctacgtgtgctgggtccgccaggcccctggcaaaggcctggagtggattgcttgtatttataccggcgacggcaccaactacagcgcaagttgggccaagggccgtttcaccatctccaaggatagtagcaagaacacggtatacctgcagatgaactctcttcgcgccgaggacaccgccgtgtatttctgtgcccggcccgacatcacttatggcttcgcgattaacttttggggccctggaaccctggtgaccgtgtcctccggcggcggtggctctggggggggcgggtccggcggaggcggcagcggaggcggcggctccgacatccagatgacccagtcgcccagttctttgtccgcttccgtgggggacagggtcaccataaagtgccaggcttctcagagcatctcgtcccgcctggcgtggtaccagcagaagcccgggaagcccccaaagctgctcatctaccgggcttccactctggccagcggtgttccttcccgcttctctggctccgggtcgggcactgactttacattgacgatctcttcgcttcagccggaagatgtagctacttactactgtcaatgcactggctacggcatcagttggcccatcggtggaggcaccaaggtggagatcaaaagcgggggaggcggttctggcggtggagggtctggcgggggtggctccgggggcggtggtagcggtggtggatccctacagctgctgccctcttgggccatcacgctcatttcagttaatggcatcttcgtcatctgctgcctgacctactgctttgcgccgaggtgtagagagcggcgtcgcaacgagcgcctgcgtcgcgagagcgtgcgcccggtg

SEQ ID NO: 315 - protein, 337 aa, synthetic construct
MDWTWILFLVAAATRVHSEVQLVESGGGLVQPGGSLRLSCAASGYSFTGYYIHWVRQAPGKGLEWVARVIPNAGGTSYNQKFKGRFTLSVDNSKNTAYLQMNSLRAEDTAVYYCAREGIYWWGQGTLVTVSSGGGGSGGGGSGGGGSGGGGSDIQMTQSPSSLSASVGDRVTITCRSSQSLVHSNGNTFLHWYQQKPGKAPKLLIYTVSNRFSGVPSRFSGSGSGTDFTLTISSLQPEDFATYFCSQTTHVPWTFGQGTKVEIKSGGGGSGGGGSGGGGSGGGGSGGGSLQLLPSWAITLISVNGIFVICCLTYCFAPRCRERRRNERLRRESVRPV

SEQ ID NO: 316 - DNA, 1011 bp, synthetic construct
  source 1..1011: mol_type = other DNA
atggattggacctggatcctgttcctggtggcggcggctacacgtgtgcactccgaggtgcagttggtggagagcggcggtggtctggtgcagccgggcgggagcctgcgcctgtcgtgcgccgcttccggctactccttcaccggttattacatccactgggtgcgacaggccccgggcaaaggcctggagtgggtcgctcgcgtaattcctaacgcgggtggcaccagctacaaccagaaatttaagggcagattcaccctcagcgttgacaactccaagaacacggcctacctgcagatgaactcccttcgcgccgaggacaccgccgtgtactactgtgctcgcgagggcatctattggtggggccagggaactctagttaccgtgtcctccggcggcggcggctcgggcggcgggggttctggtgggggcggttccggagggggagggtccgacatccagatgacccagagcccctctagtctgagcgcatccgtgggggacagggtcaccatcacctgccgctcgtcacaatcgcttgtccactcaaatggcaacactttcctgcattggtaccagcagaagcccggcaaggcgcccaagctgctcatctacaccgtgagcaaccgcttttcaggcgtgccatcccgcttctctggctccggctccggtacagatttcacccttaccattagctcgttgcagccagaagactttgccacctatttctgttctcagacgactcacgtcccgtggaccttcggacagggcaccaaggtggagatcaagagcggcggaggtgggagtggaggcggcgggtccggagggggcggttctggcggcggtggttctggcggagggtctctgcagctgctacccagttgggccatcactctcatttctgtcaacggcatcttcgtcatatgttgcctgacctactgctttgctcctaggtgtcgtgagcgccgccgcaacgagcggttgcggcgtgaatccgtgcgccctgta

SEQ ID NO: 317 - protein, 343 aa, synthetic construct
MDWTWILFLVAAATRVHSQVQLVESGGGVVQPGRSLRLSCAASGFTFSSYGMHWVRQAPGKGLEWVAVISYEESNRYHADSVKGRFTISRDNSKITLYLQMNSLRTEDTAVYYCARDGGIAAPGPDYWGQGTLVTVSSGGGGSGGGGSGGGGSGGGGSDIVMTQSPLSLTVTPGEPASISCRSSQSLLYSNGYNYLDWYLQKPGQSPQVLISLGSNRASGVPDRFSGSGSGTDFTLKISRVEAEDVGVYYCMQARQTPFTFGPGTKVDIRSGGGGSGGGGSGGGGSGGGGSGGGSLQLLPSWAITLISVNGIFVICCLTYCFAPRCRERRRNERLRRESVRPV

SEQ ID NO: 318 - DNA, 1029 bp, synthetic construct
  source 1..1029: mol_type = other DNA
atggactggacctggatcctgttcctggtcgcagcggccacccgcgtacattctcaggtgcagctggtggaatccgggggtggcgtggtgcaaccaggccgtagcctgcgcctgagctgtgctgcgtccggctttactttttcgtcttacggcatgcactgggtgcgccaggccccgggtaaaggcttggagtgggtcgcggttatcagctatgaggagagcaaccgctaccacgcggactccgtgaagggccgattcaccatctcgcgggacaactccaagataaccctgtacctgcagatgaactctctgcggactgaagacaccgccgtgtactactgtgcccgcgatggcggaatcgccgctcccgggcccgactattggggccagggcacactcgttaccgtctcctcgggaggcggcggcagtggtggtggaggttccggcggcgggggctccgggggtggcggctctgacatcgtgatgacgcagagccctttgagcctaacagttacccccggggagcccgcttcgatttcatgccgctcctcccagagcctgctgtactcaaatggctacaactacctggactggtacctccagaagccgggtcagtcccctcaggtgctcatctcgttgggcagcaacagggcctctggcgtgccagaccgcttctccggatctggcagcggaaccgatttcaccctgaagatctctagagtggaggctgaggacgtgggcgtgtattactgtatgcaggcccgacagactcccttcaccttcggccccgggaccaaggtggatatccgctcgggaggcggcggcagcggtgggggaggcagcggtggtggcggatccggggggggcggctctggtggcggatccctccagctgctgccgagttgggctatcactcttatttccgtaaatgggattttcgtcatctgctgccttacctactgctttgcacctagatgtcgcgagcgtaggcgcaacgagcggcttcgtcgcgagtccgtccgccctgtc

SEQ ID NO: 319 - protein, 344 aa, synthetic construct
MDWTWILFLVAAATRVHSQVQLVQSGAEVKKPGASVKVSCKASGYTFTGYYMHWVRQAPGQGLEWMGWINPDSGGTNYAQKFQGRVTMTRDTSISTAYMELNRLRSDDTAVYYCARDQPLGYCTNGVCSYFDYWGQGTLVTVSSGGGGSGGGGSGGGGSGGGGSDIQMTQSPSSVSASVGDRVTITCRASQGIYSWLAWYQQKPGKAPNLLIYTASTLQSGVPSRFSGSGSGTDFTLTISSLQPEDFATYYCQQANIFPLTFGGGTKVEIKSGGGGSGGGGSGGGGSGGGGSGGGSLQLLPSWAITLISVNGIFVICCLTYCFAPRCRERRRNERLRRESVRPV

SEQ ID NO: 320 - DNA, 1032 bp, synthetic construct
  source 1..1032: mol_type = other DNA
atggattggacctggatcctgttcctggtggctgctgccacccgtgtgcacagccaggtgcagctggtgcaatccggcgccgaggtgaagaagcccggcgccagcgtgaaggtatcgtgtaaggcttcgggctacacgttcaccggctactacatgcattgggtccgccaggcccccggacagggcctggagtggatgggctggattaaccccgactccggcggcaccaactacgcgcagaaatttcaggggcgagtgactatgacaagggacacttccatctccacagcctacatggagctcaaccgcctgcgcagcgatgacaccgcagtgtactattgtgctcgcgaccagcctctaggttactgcactaatggcgtatgtagctatttcgactattggggccagggcaccctggtcaccgtgtccagcggcggagggggctcaggcggaggtggctcaggaggtggcggctcggggggcggtgggtccgacattcagatgacccagtccccgagctcggtctctgcttctgtgggagaccgcgtgacgataacctgccgtgcctcccagggcatctacagttggcttgcgtggtaccagcagaagccaggcaaggcgccaaacctgctgatttacaccgcatccactcttcagtcaggcgttccttcccgcttcagtggctctggttctggcaccgatttcacgctgaccatctcctccttgcagccggaagactttgccacttactactgccaacaggccaacatcttccctctgaccttcggcggtggcaccaaggtggagatcaaatctggcggagggggctcgggcgggggtggttctggcggcgggggaagtggtgggggcggatctggtggtggtagcctgcagctcttgccctcctgggcgatcaccctcatcagcgtcaatggcatcttcgtcatctgttgcctgacctactgctttgcgccgagatgtcgagagcgccggcgcaacgagcgcctgcgtcgggagtccgtgcgccccgtg

SEQ ID NO: 321 - DNA, 783 bp, synthetic construct
  source 1..783: mol_type = other DNA
atgatcgagacctacaaccagacttccccacgtagcgccgccactggtctacccatctcgatgaaaatcttcatgtatctgctgaccgtgttcctgatcacccagatgattggctccgctctgtttgccgtgtacttgcaccgccgcctggacaaaattgaggacgagcggaatctgcacgaagatttcgtatttatgaagaccatccagaggtgtaacactggcgagcgttctctttcgttgcttaactgcgaagagatcaagagccagttcgagggcttcgtcaaggacatcatgctcaacaaggaggagactaagaaggagaactcctttgagatgcagaagggtgatcagaacccccaaattgcagcgcatgtgataagcgaggcttcctccaagacaaccagtgtgctgcagtgggcggagaagggctactacaccatgtcaaataacctggtgaccctggagaacggcaagcagctgacggtgaagcgccaggggctctattacatctacgcgcaggtcacgttctgttcgaaccgcgaagcctcctctcaggctcccttcatcgcctcactgtgcctgaaatctccgggacgcttcgagcgaatcctgctccgcgccgccaacacgcactcctccgccaagccttgcgggcagcagagcatccaccttggtggggtttttgagctgcagcctggcgcgtccgtgttcgtcaacgtgaccgacccgtcccaggtctctcatggaaccggcttcacctccttcggcctgctcaagttg

SEQ ID NO: 322 - protein, 566 aa, synthetic construct
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKLSGGGGSGGGGSGGGGSGGGGSGGGSLQLLPSWAITLISVNGIFVICCLTYCFAPRCRERRRNERLRRESVRPVRAKRSGSGATNFSLLKQAGDVEENPGPMDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGGGGSGGGGSGGGSLQLLPSWAITLISVNGIFVICCLTYCFAPRCRERRRNERLRRESVRPV

SEQ ID NO: 323 - DNA, 1698 bp, synthetic construct
  source 1..1698: mol_type = other DNA
atgatcgaaacttacaaccagacgagcccgcgctccgccgccaccgggctgcccatctctatgaaaatcttcatgtatctgctgaccgtgtttctgatcacccagatgatcggttccgcgctgtttgccgtgtacctgcaccgccgcctggacaagatagaagacgagcgcaacctgcacgaagatttcgtcttcatgaagaccatccagcggtgtaataccggcgagaggagcctgagcttgctcaactgcgaggagatcaaaagtcagttcgagggcttcgttaaggacatcatgctcaacaaggaggagaccaagaaggagaactccttcgagatgcaaaagggggaccagaaccctcagatcgccgcgcatgtaatttccgaggcctcatccaagacgacttctgtcctacagtgggcagagaagggctactacaccatgtctaataacctggtcacccttgagaacggcaagcaactgacagtgaagcgccagggcctctactacatctacgcgcaggtgaccttctgcagcaaccgcgaggcttcctcccaagctcccttcatcgcatctctgtgcctgaaaagccctggccgcttcgagcggattctccttcgtgcagcaaacactcactcgtccgccaagccctgtggacagcagagcatccacctgggcggcgtgttcgagctgcagccaggcgcctccgtgttcgtcaatgtgaccgacccgtcccaggtgtcccatggcactggcttcacctccttcggcctcctcaaactgtccggcggcggtggcagtggtggcgggggatcaggtggcggcggctccggaggtggcgggtccgggggaggttctcttcagcttctgcctagctgggcgatcaccctgatctccgttaatggcatcttcgtcatttgttgtttgacctattgctttgccccgagatgtcgagagcgtagacgcaacgagcgcctgcgccgtgaaagcgtgcgacctgtgcgcgctaagcgcagcggctccggcgctaccaacttctcattactcaagcaggctggagatgtagaggagaatccagggccaatggattggacctggatcctgttcctggtggccgccgcgactcgcgtccacagtaattgggtcaacgtgatctctgacctgaaaaagattgaggacttgatccagagcatgcacattgacgctaccctgtacacggagagtgacgtgcacccctcgtgcaaggtgacagccatgaagtgctttttgctggagttgcaggtcatctctctagagagcggtgatgcctccatccatgacaccgtggagaaccttatcatcctggccaacaacagcctgtcgagcaacggcaacgtgaccgagtccgggtgcaaggaatgtgaggagctggaggagaaaaacatcaaggagttcctgcagtcgtttgtgcacatcgtgcagatgtttattaacacctcttctggcggtgggggatcgggggggggcggctccggcggtggcggttctggaggcggtggttctggcgggggatctttacagctgctgcccagttgggcgatcactctcatttccgtgaacggcattttcgtcatttgctgtttgacctactgctttgctccgaggtgtcgcgagcgtcgccgcaacgagcgcctacggcgggaatcggtgaggcccgtg

SEQ ID NO: 324 - protein, 226 aa, synthetic construct
MYRMQLLSCIALSLALVTNSAPTSSSTKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLTSGGGGSGGGGSGGGGSGGGGSGGGSLQLLPSWAITLISVNGIFVICCLTYCFAPRCRERRRNERLRRESVRPV

SEQ ID NO: 325 - DNA, 681 bp, synthetic construct
  source 1..681: mol_type = other DNA
atgtatcggatgcagctgctgtcttgtattgctctatcactggcgctggtgaccaactccgctcctacttccagctcgaccaagaagacccagctccaacttgagcacctgctgcttgatcttcagatgatcctgaacggcatcaacaactacaaaaaccccaagctgacccgcatgctcactttcaagttctacatgcctaaaaaggccaccgagctcaagcacttgcagtgcctggaggaggagttgaagccgttggaagaggtgctcaacctggcccagtccaagaatttccatctaagaccccgcgacctgatctctaacatcaacgtgattgtactggagctgaagggctccgagaccaccttcatgtgcgagtacgcggacgagactgcaaccatcgtggagttcctgaaccgctggatcacgttctgtcagagcatcatctcgacccttacatctggcggcggggggtcgggcggtgggggctctggaggaggcggtagtgggggtggcggctccggtggaggcagcctgcagctgctcccaagttgggccatcaccctgattagcgtcaatggcatcttcgtcatatgttgtctgacctactgctttgccccccgctgccgagaacgtcgccgcaacgagcggctgcgcagggagtccgtgcgcccggtgtga

SEQ ID NO: 326 - protein, 601 aa, synthetic construct; identical to SEQ ID NO: 303

SEQ ID NO: 327 - DNA, 681 bp, synthetic construct; identical to SEQ ID NO: 325
  source 1..681: mol_type = other DNA

SEQ ID NO: 328 - protein, 205 aa, synthetic construct
MDWTWILFLVAAATRVHSNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTSSGGGGSGGGGSGGGGSGGGGSGGGSLQLLPSWAITLISVNGIFVICCLTYCFAPRCRERRRNERLRRESVRPV

SEQ ID NO: 329 - DNA, 618 bp, synthetic construct
  source 1..618: mol_type = other DNA
atggattggacctggatcctgttcctggtggctgcggccacccgtgtacacagcaactgggtcaatgtgatctccgacctgaagaagattgaggacctcatccagtctatgcacattgacgcaacactttacaccgagagcgatgtgcacccctcttgcaaggtgactgccatgaaatgtttcttgctcgaattgcaggtcatctctctggagagtggggacgcttccatacatgacaccgtggagaacctgatcatcctggccaacaacagcctctcgtcaaatggcaacgtgactgagagcgggtgcaaagagtgtgaggagctggaggagaagaacatcaaggagttcctgcagagcttcgtccacatcgtgcagatgtttattaacacgtcgtccggaggcgggggctctggtggcggcggctccgggggaggcggctccggtggcggtggttccggcggaggctcgctacagctgctgcccagttgggccatcacccttatttctgtcaacggcatcttcgttatctgttgcctgacctactgctttgcgccgaggtgtcgcgagcggcgtcgcaacgagcgcctgcgccgagaatccgtgcgccctgtgtga

SEQ ID NO: 330 - DNA, 717 bp, synthetic construct
  source 1..717: mol_type = other DNA
atgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactcacggcatggacgagctgtacaagtga

SEQ ID NO: 331 - protein, 157 aa, synthetic construct
YFGKLESKLSVIRNLNDQVLFIDQGNRPLFEDMTDSDSRDNAPRTIFIISKYSDSLARGLAVTISVKSEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 332 - protein, 157 aa, synthetic construct
YFGKLESKLSVIRNLNDQVLFIDQGNRPLFEDMTDSDSRDNAPRTIFIISKYSDSLARGLAVTISVKGEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 333 - protein, 157 aa, synthetic construct
YFGKLESKLSVIRNLNDQVLFIDQGNRPLFEDMTDSDSRDNAPRTIFIISKYSDSLARGLAVTISVKAEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 334 - protein, 157 aa, synthetic construct
YFGKLESKLSVIRNLNDQVLFIDQGNRPLFEDMTDSDSRDNAPRTIFIISKYSDSLARGLAVTISVKVEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 335 - protein, 157 aa, synthetic construct
YFGKLESKLSVIRNLNDQVLFIDQGNRPLFEDMTDSDSRDNAPRTIFIISKYSDSLARGLAVTISVKDEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 336 - protein, 157 aa, synthetic construct
YFGKLESKLSVIRNLNDQVLFIDQGNRPLFEDMTDSDSRDNAPRTIFIISKYSDSLARGLAVTISVKEEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 337 - protein, 144 aa, synthetic construct
YFGKLESKLSVIRNLNDQVLFIDQGNRPLFEDMTDSDSRDNAPRTIFIISKYSDSLARGLAVTISVKNEKISTLSCENKIISFKEMNPPDNIKDTKSDIIFFQRDVPGHSRKMQFESSSYEGYFLACEKERDLFKLILKKEDEL

SEQ ID NO: 338 - protein, 157 aa, synthetic construct
YFGKLESKLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 339 - protein, 157 aa, synthetic construct
YFGKLESKLSVIRNLNDQVLFIDQGNRPLFEDMTDSDCRDNAPRTIFIISTYKDKQPRAKAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 340 - protein, 157 aa, synthetic construct
RFGKLESKLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 341 - protein, 157 aa, synthetic construct
RFGKLESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRNVPGHKYKMQFESSSYEGYFLACEKERDLFKLILKKEDELGDRSIMFTVQNED

SEQ ID NO: 342 - protein, 157 aa, synthetic construct
YFGKLESQLSVIRNLNDQVLFIDQGNRPLFEDMTDSDCRDNAPRTIFIISTYKDKQPRTKAVTISVKCEKISTLSCENKIISFKEMNPPDNIKDTKSDIIFFQRRVPGHHNKMQFESSSYEGYFLACEKERDLFKLILKKEDELGDRSIMFTVQKED

SEQ ID NO: 343 - protein, 157 aa, synthetic construct
YFGKLESRLSVIRNLNDQVLFIDQGNRPLFEDMTDSDCRDNAPRTIFIISKYKDKQPRAQAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 344 - protein, 158 aa, synthetic construct
YFGKLESRLSVIRNLNDQVLFIDQGNRPLFEDMTDSDCRDNAPRTIFIISDYKDKQPRAXAAVTISVKCEKISTLSCENKIISFKEMNPPDNIKDTKSDIIFFQRDVPGHKHKMQFESSSYEGYFLACEKERDLFKLILKKEDELGDRSIMFTIQNED

SEQ ID NO: 345 - protein, 157 aa, synthetic construct
YFGKHESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHNNKMQFESSSYEGYFLACEKERDLFKLILKKEDELGDRSIMFTTQNED

SEQ ID NO: 346 - protein, 157 aa, synthetic construct
YFGKIESKLSVIRNLNDQVLFIDQGNRPLFEDMTDSDCRDNAPRTIFIISKYKDKQPRAQAVTISVKCEKISTLSCENKIISFKEMNPPDNIKDTKSDIIFFQRKVPGHQHKMQFESSSYEGYFLACEKERDLFKLILKKEDELGDRSIMFTVQKED

SEQ ID NO: 347 - protein, 157 aa, synthetic construct
YFGKIESKLSVIRNLNDQVLFIDQGNRPLFEDMTDSDCRDNAPRTIFIISTYKDRQPRGKAVTISVKCEKISTLSCENKIISFKEMNPPDNIKDTKSDIIFFERDVPGHHHKMQFESSSYEGYFLACEKERDLFKLILKKEDELGDRSIMFTIQNED

SEQ ID NO: 348 - protein, 157 aa, synthetic construct
YFGKIESKLSVIRNLNDQVLFIDQGNRPLFEDMTDSDCRDNAPRTIFIISTYKDKQPRGKAVTISVKCEKISTLSCENKIISFKEMNPPDNIKDTKSDIIFFQRDVPGHKHKMQFESSSYEGYFLACEKERDLFKLILKKEDELGDRSIMFTTQHED

SEQ ID NO: 349 - protein, 157 aa, synthetic construct
YFGKIESKLSVIRNLNDQVLFIDQGNRPLFEDMTDSDCRDNAPRTIFIISTYKDKQPRAKAVTISVKCEKISTLSCENKIISFKEMNPPDNIKDTKSDIIFFQRRVPGHHHKMQFESSSYEGYFLACEKERDLFKLILKKEDELGDRSIMFTIQKED

SEQ ID NO: 350 - protein, 157 aa, synthetic construct
YFGKIESRLSVIRNLNDQVLFIDQGNRPLFEDMTDSDCRDNAPRTIFIISTYKDKQPRGKAVTISVKCEKISTLSCENKIISFKEMNPPDNIKDTKSDIIFFQRDVPGHDYKMQFESSSYEGYFLACEKERDLFKLILKKEDELGDRSIMFTIQKED

SEQ ID NO: 351 - protein, 157 aa, synthetic construct
YFGKYESRLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHEHKMQFESSSYEGYFLACEKERDLFKLILKKEDELGDRSIMFTVQKED

SEQ ID NO: 352 - protein, 157 aa, synthetic construct
HFGKYESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFQRDVPGHHNKMQFESSSYEGYFLACEKERDLFKLILKKEDELGDRSIMFTVQKED

SEQ ID NO: 353 - protein, 157 aa, synthetic construct
RFGKLESKLSVIRNLNDQVLFIDQGNRPLFEDMTDSDCRDNAPRTIFIISTYRDSQPRAKAVTISVKCEKISTLSCENKIISFKEMNPPDNIKDTKSDIIFFQRDVPGHQHKMQFESSSYEGYFLACEKERDLFKLILKKEDELGDRSIMFTAQKED

SEQ ID NO: 354 - protein, 157 aa, synthetic construct
RFGKLESRLSVIRNLNDQVLFIDQGNRPLFEDMTDSDCRDNAPRTIFIISDYRDSQPRGRAVTISVKCEKISTLSCENKIISFKEMNPPDNIKDTKSDIIFFKRNVPGHKYKMQFESSSYEGYFLACEKERDLFKLILKKEDELGDRSIMFTVQHED

SEQ ID NO: 355 - protein, 157 aa, synthetic construct
RFGKLESRLSVIRNLNDQVLFIDQGNRPLFEDMTDSDCRDNAPRTIFIISNYRDSQPRGQAVTISVKCEKISTLSCENKIISFKEMNPPDNIKDTKSDIIFFKRRVPGHNHKMQFESSSYEGYFLACEKERDLFKLILKKEDELGDRSIMFTVQKED

SEQ ID NO: 356 - protein, 157 aa, synthetic construct
RFGKLESRLSVIRNLNDQVLFIDQGNRPLFEDMTDSDCRDNAPRTIFIISTYKDSQPRGKAVTISVKCEKISTLSCENKIISFKEMNPPDNIKDTKSDIIFFQRDVPGHKHKMQFESSSYEGYFLACEKERDLFKLILKKEDELGDRSIMFTVQNED

SEQ ID NO: 357 - protein, 157 aa, synthetic construct
RFGKHESKLSVIRNLNDQVLFIDQGNRPLFEDMTDSDCRDNAPRTIFIISTYRDSQPRGKAVTISVKCEKISTLSCENKIISFKEMNPPDNIKDTKSDIIFFERNVPGHKYKMQFESSSYEGYFLACEKERDLFKLILKKEDELGDRSIMFTVQNED

SEQ ID NO: 358 - protein, 194 aa, synthetic construct
RFGKLESRLSVIRNLNDQVLFIDQGNRPLFEDMTDSDCRDNAPRTIFIISTYRDSQPRAKAVTISVKCEKISTLSCENKIISFKEMNPPDNIKDTKSDIIFFERDVPGHQHKMQFESSSYEGYFLACEKERDLFKLILKKEDELGDRSIMFTIQXEDSEQIDNOXANYNATURALLYOCCURINGAMINOACID

SEQ ID NO: 359 - protein, 157 aa, synthetic construct
RFGKLESRLSVIRNLNDQVLFIDQGNRPLFEDMTDSDCRDNAPRTIFIISTYRDSQPRTKAVTISVKCEKISTLSCENKIISFKEMNPPDNIKDTKSDIIFFQRNVPGHHDKMQFESSSYEGYFLACEKERDLFKLILKKEDELGDRSIMFTVQHED

SEQ ID NO: 360 - protein, 157 aa, synthetic construct
RFGKLESRLSVIRNLNDQVLFIDQGNRPLFEDMTDSDCRDNAPRTIFIISTYKDSQPRAKAVTISVKCEKISTLSCENKIISFKEMNPPDNIKDTKSDIIFFERDVPGHQHKMQFESSSYEGYFLACEKERDLFKLILKKEDELGDRSIMFTIQKED

SEQ ID NO: 361 - protein, 157 aa, synthetic construct; identical to SEQ ID NO: 357

SEQ ID NO: 362 - protein, 157 aa, synthetic construct
RFGKYESKLSVIRNLNDQVLFIDQGNRPLFEDMTDSDCRDNAPRTIFIISTYKDSQPRTKAVTISVKCEKISTLSCDNKIISFKEMNPPDNIKDTKSDIIFFQRDVPGHKHKMQFESSSYEGYFLACEKERDLFKLILKKEDELGDRSIMFTVQNED

SEQ ID NO: 363 - protein, 157 aa, synthetic construct
RFGKLESRLSVIRNLNDQVLFIDQGNRPLFEDMTDSDCRDNAPRTIFIISTYRDSQPRTKAVTISVKCEKISTLSCENKIISFKEMNPPDNIKDTKSDIIFFQRKVPGHNHKMQFESSSYEGYFLACEKERDLFKLILKKEDELGDRSIMFTVQKED

SEQ ID NO: 364 - protein, 157 aa, synthetic construct
YFGKLESKLSVIRNLNDQVLFIDQGNRPLFEDMTDSDCRDNAPRTIFIISEYKDSELRGRAVTISVKCEKISTLSCENKIISFKEMNPPDNIKDTKSDIIFFPRAVPGHNRKVQFESSSYEGYFLACEKERDLFKLILKKEDELGDRSIMFTVQNED

SEQ ID NO: 365 - protein, 157 aa, synthetic construct
YFGKLESKLSVIRNLNDQVLFIDQGNRPLFEDMTDSDCRDNAPRTIFIISKYKDSAGRGLAVTISVKCEKISTLSCENKIISFKEMNPPDNIKDTKSDIIFFERDVPGHSNKVQFESSSYEGYFLACEKERDLFKLILKKEDELGDRSIMFTVQNED

SEQ ID NO: 366 - protein, 157 aa, synthetic construct
YFGKLESKLSVIRNLNDQVLFIDQGNRPLFEDMTDSDCRDNAPRTIFIISKYGDSAARGLAVTISVKCEKISTLSCENKIISFKEMNPPDNIKDTKSDIIFFQRSVPGHKRKMQFESSSYEGYFLACEKERDLFKLILKKEDELGDRSIMFTVQNED

SEQ ID NO: 367 - protein, 157 aa, synthetic construct
YFGKLESKLSVIRNLNDQVLFIDQGNRPLFEDMTDSDCRDNAPRTIFIISKYGDSRGRGLAVTISVKCEKISTLSCENKIISFKEMNPPDNIKDTKSDIIFFERDVPGHNSKRQFESSSYEGYFLACEKERDLFKLILKKEDELGDRSIMFTVQNED

SEQ ID NO: 368 - protein, 157 aa, synthetic construct
YFGKLESKLSVIRNLNDQVLFIDQGNRPLFEDMTDSDCRDNAPRTIFIISKYGDSVPRGLAVTISVKCEKISTLSCENKIISFKEMNPPDNIKDTKSDIIFFARAVPGHSRKTQFESSSYEGYFLACEKERDLFKLILKKEDELGDRSIMFTVQNED

SEQ ID NO: 369 - protein, 157 aa, synthetic construct
YFGKLESKLSVIRNLNDQVLFIDQGNRPLFEDMTDSDCRDNAPRTIFIISKYSDSGARGLAVTISVKCEKISTLSCENKIISFKEMNPPDNIKDTKSDIIFFARAVPGHGRKTQFESSSYEGYFLACEKERDLFKLILKKEDELGDRSIMFTVQNED

SEQ ID NO: 370 - protein, 157 aa, synthetic construct
YFGKLESKLSVIRNLNDQVLFIDQGNRPLFEDMTDSDCRDNAPRTIFIISKYSDSKARGMAVTISVKCEKISTLSCENKIISFKEMNPPDNIKDTKSDIIFFARDVPGHSSKRQFESSSYEGYFLACEKERDLFKLILKKEDELGDRSIMFTVQNED

SEQ ID NO: 371 - protein, 157 aa, synthetic construct
YFGKLESKLSVIRNLNDQVLFIDQGNRPLFEDMTDSDCRDNAPRTIFIISKYSDSLARGLAVTISVKCEKISTLSCENKIISFKEMNPPDNIKDTKSDIIFFQRDVPGHSRKMQFESSSYEGYFLACEKERDLFKLILKKEDELGDRSIMFTVQNED

SEQ ID NO: 372 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFQRNVPGHGRKMQFESSSYEGYFLACEKERDLFKLILKKEDELGDRSIMFTVQNED

SEQ ID NO: 373 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARSVPGHGRKTQFESSSYEGYFLACEKERDLFKLILKKEDELGDRSIMFTVQNED

SEQ ID NO: 374 - protein, 157 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARDVPGHSGKRQFESSSYEGYFLACEKERDLFKLILKKEDELGDRSIMFTVQNED

SEQ ID NO: 375 - protein, 157 aa, synthetic construct
YFGKLESKLSVIRNLNDQVLFIDQGNRPLFEDMTDSDCRDNAPRTIFIISKYTDSRPRGLAVTISVKCEKISTLSCENKIISFKEMNPPDNIKDTKSDIIFFERDVPGHSSKKQFESSSYEGYFLACEKERDLFKLILKKEDELGDRSIMFTVQNED

SEQ ID NO: 376 - protein, 157 aa, synthetic construct
YFGKLESKLSVIRNLNDQVLFIDQGNRPLFEDMTDSDCRDNAPRTIFIISKYTDSRARGMAVTISVKCEKISTLSCENKIISFKEMNPPDNIKDTKSDIIFFERDVPGHNDKRQFESSSYEGYFLACEKERDLFKLILKKEDELGDRSIMFTVQNED

SEQ ID NO: 377 - protein, 157 aa, synthetic construct
YFGKLESKLSVIRNLNDQVLFIDQGNRPLFEDMTDSDCRDNAPRTIFIISRYKDSGKRGLAVTISVKCEKISTLSCENKIISFKEMNPPDNIKDTKSDIIFFRRSVPGHSRKVQFESSSYEGYFLACEKERDLFKLILKKEDELGDRSIMFTVQNED

SEQ ID NO: 378 - protein, 157 aa, synthetic construct
YFGKLESKLSVIRNLNDQVLFIDQGNRPLFEDMTDSDCRDNAPRTIFIISKYGDSGARGLAVTISVKCEKISTLSCENKIISFKEMNPPDNIKDTKSDIIFFERDVPGHSGKVQFESSSYEGYFLACEKERDLFKLILKKEDELGDRSIMFTVQNED

SEQ ID NO: 379 - protein, 157 aa, synthetic construct
YFGKLESKLSVIRNLNDQVLFIDQGNRPLFEDMTDSDCRDNAPRTIFIISKYGDSRPRGMAVTISVKCEKISTLSCENKIISFKEMNPPDNIKDTKSDIIFFQRAVPGHNRKMQFESSSYEGYFLACEKERDLFKLILKKEDELGDRSIMFTVQNED

SEQ ID NO: 380 - protein, 157 aa, synthetic construct; identical to SEQ ID NO: 371

SEQ ID NO: 381 - protein, 156 aa, synthetic construct
YFGKLESKLSVIRNLNDQVLFIDQGNRPLFEDMTDSDCRDNAPRTIFIISKYSDSRARGLAVTISVKCEKISTLSCENKIISFKEMNPPDNIKDTKSDIIFFARSVPGHGRKTQFESSSYEGYFLACEKERDLFKLILKKEDELGDRSIMFTVQNE

SEQ ID NO: 382 - protein, 157 aa, synthetic construct; identical to SEQ ID NO: 372

SEQ ID NO: 383 - protein, 157 aa, synthetic construct; identical to SEQ ID NO: 368

SEQ ID NO: 384 - protein, 157 aa, synthetic construct; identical to SEQ ID NO: 373

SEQ ID NO: 385 - protein, 157 aa, synthetic construct; identical to SEQ ID NO: 374

SEQ ID NO: 386 - protein, 199 aa, synthetic construct
EGIKSNISLLKDELRGQISHISHEYLSLIDLAFDSKQNRLFEMKVLELLVNEYGFKGRHLGGSRKPDGIVYSTTLEDNFGIIVDTKAYSEGYSLPISQADEMERYVRENSNRDEEVNPNKWWENFSEEVKKYYFVFISGSFKGKFEEQLRRLSMTTGVNGSAVNVVNLLLGAEKIRSGEMTIEELERAMFNNSEFILKY

SEQ ID NO: 387 - protein, 594 aa, synthetic construct
MGSSLDDEHILSALLQSDDELVGEDSDSEISDHVSEDDVQSDTEEAFIDEVHEVQPTSSGSEILDEQNVIEQPGSSLASNRILTLPQRTIRGKNKHCWSTSKSTRRSRVSALNIVRSQRGPTRMCRNIYDPLLCFKLFFTDEIISEIVKWTNAEISLKRRESMTGATFRDTNEDEIYAFFGILVMTAVRKDNHMSTDDLFDRSLSMVYVSVMSRDRFDFLIRCLRMDDKSIRPTLRENDVFTPVRKIWDLFIHQCIQNYTPGAHLTIDEQLLGFRGRCPFRMYIPNKPSKYGIKILMMCDSGTKYMINGMPYLGRGTQTNGVPLGEYYVKELSKPVHGSCRNITCDNWFTSIPLAKNLLQEPYKLTIVGTVRSNKREIPEVLKNSRSRPVGTSMFCFDGPLTLVSYKPKPAKMVYLLSSCDEDASINESTGKPQMVMYYNQTKGGVDTLDQMCSVMTCSRKTNRWPMALLYGMINIACINSFIIYSHNVSSKGEKVQSRKKFMRNLYMSLTSSFMRKRLEAPTLKRYLRDNISNILPNEVPGTSDDSTEEPVMKKRTYCTYCPSKIRRKANASCKKCKKVICREHNIDMCQSCF

SEQ ID NO: 388 - protein, 340 aa, synthetic construct
MGKSKEISQDLRKKIVDLHKSGSSLGAISKRLKVPRSSVQTIVRKYKHHGTTQPSYRSGRRRVLSPRDERTLVRKVQINPRTTAKDLVKMLEETGTKVSISTVKRVLYRHNLKGRSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMRKENYVDILKQHLKTSVRKLKLGRKWVFQMDNDPKHTSKVVAKWLKDNKVKVLEWPSQSPDLNPIENLWAELKKRVRARRPTNLTQLHQLCQEEWAKIHPTYCGKLVEGYPKRLTQVKQFKGNATKY

SEQ ID NO: 389 - protein, 340 aa, synthetic construct
MGKSKEISQDLRKRIVDLHKSGSSLGAISKRLAVPRSSVQTIVRKYKHHGTTQPSYRSGRRRVLSPRDERTLVRKVQINPRTTAKDLVKMLEETGTKVSISTVKRVLYRHNLKGHSARKKPLLQNRHKKARLRFATAHGDKDRTFWRNVLWSDETKIELFGHNDHRYVWRKKGEACKPKNTIPTVKHGGGSIMLWGCFAAGGTGALHKIDGIMDAVQYVDILKQHLKTSVRKLKLGRKWVFQHDNDPKHTSKVVAKWLKDNKVKVLEWPSQSPDLNPIENLWAELKKRVRARRPTNLTQLHQLCQEEWAKIHPNYCGKLVEGYPKRLTQVKQFKGNATKY

SEQ ID NO: 390 - DNA, 239 bp, synthetic construct
  source 1..239: mol_type = other DNA
ggaggaaaaactgtttcatacagaaggcgtcaattaggaggaaaaactgtttcatacagaaggcgtcaattaggaggaaaaactgtttcatacagaaggcgtcaattggtcccatcgaattaggaggaaaaactgtttcatacagaaggcgtcaattaggaggaaaaactgtttcatacagaaggcgtcaattaggaggaaaaactgtttcatacagaaggcgtcaattggtcccggga

SEQ ID NO: 391 - DNA, 113 bp, synthetic construct
  source 1..113: mol_type = other DNA
cattttgacacccccataatatttttccagaattaacagtataaattgcatctcttgttcaagagttccctatcactctctttaatcactactcacagtaacctcaactcctg

SEQ ID NO: 392 - DNA, 1806 bp, synthetic construct
  source 1..1806: mol_type = other DNA
atggattggacctggattctgttcctcgtggccgccgctacccgcgtgcactccatctgggagctgaagaaagacgtgtacgtggtggaattggattggtaccccgacgccccaggcgagatggtggttctgacttgcgacacgccggaggaggacggcatcacctggacgctggaccagagcagcgaagtgctgggctctggtaagaccttaaccatccaggtcaaggagttcggtgatgcaggacagtacacgtgtcacaagggcggggaggtcttgtcccatagcctgctgctcctgcacaagaaggaagacggcatctggtctacggatatccttaaagaccagaaggagcccaagaacaaaaccttcctgcgatgcgaggctaagaattactccgggcgctttacctgttggtggctcacaaccatctcgaccgacctgaccttctccgtgaagtcttccaggggatcttctgatccgcaaggcgttacctgcggggccgctaccctgtcggctgagcgcgtccgtggtgacaacaaggagtacgagtactcggtggaatgccaggaggacagcgcctgcccagcggccgaagagtccttgcccatcgaggtgatggtggacgctgtacacaagctcaagtacgagaactacacctcctccttctttatacgcgacatcatcaagcctgacccccctaaaaacctgcagctgaagccccttaaaaactctcgacaggtcgaggtatcctgggagtaccccgacacctggtctactcctcattcgtatttcagcctgaccttctgcgtgcaggtgcagggcaagtccaagcgcgagaagaaggaccgcgtgttcaccgacaaaaccagcgcgactgtcatttgtcggaagaacgcgagcatctccgtacgggcacaggatcgctattactccagcagttggtcagagtgggcttcggttccgtgttctggaggaggtggcggcggctcacgcaacctgcccgtggcgacgccggacccgggtatgtttccttgtctgcaccactctcagaacttgctccgtgccgtctcgaatatgctgcagaaggcccgccagactctggagttctatccatgcactagtgaggagatcgatcatgaggacatcaccaaggacaagacatccaccgtggaggcttgtctgccgctggagttgacaaagaacgagagctgcctcaatagccgcgagacttccttcatcaccaacggctcctgcctcgcgagccgtaagacctcctttatgatggcgctgtgcctgtcaagtatctacgaagatctgaaaatgtatcaggtggagttcaaaactatgaacgcaaaactcctgatggatcccaagcgccaaattttcttggaccagaacatgcttgccgtgatcgacgagctgatgcaggccctgaacttcaactccgagaccgtccctcagaagtcgtctctggaggaacccgacttttacaagacaaagatcaagctgtgcatcctgctacacgcgttccgcattcgggccgtgaccattgaccgcgtgatgtcctacctgaatgcctcgtccggcggggggggctccggtgggggcggctctggcggaggcggctcaggtggcggcggctccggcggaggttcgcttcagcttctgcccagttgggcaatcactctgatttccgtcaacggaatcttcgtcatctgctgcctgacctactgctttgctcccagatgtagagagcgccgccgcaacgagcgcctgcgtcgtgagagcgtgaggcctgtgtga

SEQ ID NO: 393 - DNA, 574 bp, synthetic construct
  source 1..574: mol_type = other DNA
cccctctccctcccccccccctaacgttactggccgaagccgcttggaataaggccggtgtgcgtttgtctatatgttattttccaccatattgccgtcttttggcaatgtgagggcccggaaacctggccctgtcttcttgacgagcattcctaggggtctttcccctctcgccaaaggaatgcaaggtctgttgaatgtcgtgaaggaagcagttcctctggaagcttcttgaagacaaacaacgtctgtagcgaccctttgcaggcagcggaaccccccacctggcgacaggtgcctctgcggccaaaagccacgtgtataagatacacctgcaaaggcggcacaaccccagtgccacgttgtgagttggatagttgtggaaagagtcaaatggctctcctcaagcgtattcaacaaggggctgaaggatgcccagaaggtaccccattgtatgggatctgatctggggcctcggtgcacatgctttacatgtgtttagtcgaggttaaaaaaacgtctaggccccccgaaccacggggacgtggttttcctttgaaaaacacgatgataa

SEQ ID NO: 394 - DNA, 249 bp, synthetic construct
  source 1..249: mol_type = other DNA
gagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 395 - DNA, 48 bp, synthetic construct
  source 1..48: mol_type = other DNA
ggatttccagtggcgagagaactcgagttctctcgccactggaaatcc

SEQ ID NO: 396 - DNA, 48 bp, synthetic construct
  source 1..48: mol_type = other DNA
cattgtctttcctagcggaatctcgagattccgctaggaaagacaatg

SEQ ID NO: 397 - DNA, 48 bp, synthetic construct
  source 1..48: mol_type = other DNA
caaggcgcagatcaaagagagctcgagctctctttgatctgcgccttg

SEQ ID NO: 398 - DNA, 48 bp, synthetic construct
  source 1..48: mol_type = other DNA
aaggcgcagatcaaagagagcctcgaggctctctttgatctgcgcctt

SEQ ID NO: 399 - DNA, 48 bp, synthetic construct
  source 1..48: mol_type = other DNA
atcggagagcttcgtgctaaactcgagtttagcacgaagctctccgat

SEQ ID NO: 400 - DNA, 48 bp, synthetic construct
  source 1..48: mol_type = other DNA
agtatgccaccattgtctttcctcgaggaaagacaatggtggcatact

SEQ ID NO: 401 - DNA, 48 bp, synthetic construct; identical to SEQ ID NO: 395
  source 1..48: mol_type = other DNA

SEQ ID NO: 402 - protein, 245 aa, synthetic construct
MDWTWILFLVAAATRVHSQGQDRHMIRMRQLIDIVDQLKNYVNDLVPEFLPAPEDVETNCEWSAFSCFQKAQLKSANTGNNERIINVSIKKLKRKPPSTNAGRRQKHRLTCPSCDSYEKKPPKEFLERFKSLLQKMIHQHLSSRTHGSEDSSGGGGSGGGGSGGGGSGGGGSGGGGSGGGGSGGGGSGGGGSGGGGSLQLLPSWAITLISVNGIFVICCLTYCFAPRCRERRRNERLRRESVRPV